(19) United States
(12) Patent Application Publication
(10) Pub. No.: US 2025/0019340 A1
(43) Pub. Date: Jan. 16, 2025

(54) NOVEL COMPOUND AND ORGANIC LIGHT-EMITTING DEVICE COMPRISING THE SAME

(71) Applicant: LG CHEM, LTD., Seoul (KR)

(72) Inventors: Sung Jae LEE, Daejeon (KR); Sung Kil HONG, Daejeon (KR); Minjun KIM, Daejeon (KR); Hyeon Soo JEON, Daejeon (KR); Joo Ho KIM, Daejeon (KR); Hyeon Jin MUN, Daejeon (KR)

(21) Appl. No.: 18/708,994

(22) PCT Filed: Oct. 31, 2022
(86) PCT No.: PCT/KR2022/016798

§ 371 Date: May 9, 2024

(30) Foreign Application Priority Data
Nov. 9, 2021 (KR) 10-2021-0153380
Oct. 26, 2022 (KR) 10-2022-0139164

(51) Int. Cl. C07C 211/60; C07B 59/00; C07C 211/54; C07D 209/86; C07D 307/91; C07D 333/76; C07D 407/12; C07D 409/12; H10K 85/60

(52) U.S. Cl. CPC C07C 211/60; C07B 59/004; C07C 211/54; C07D 209/86; C07D 307/91; C07D 333/76; C07D 407/12; C07D 409/12; H10K 85/615; H10K 85/631; H10K 85/653; H10K 85/655; H10K 85/6572; H10K 85/6574; H10K 85/6576; C07B 2200/05

(57) ABSTRACT

The present disclosure relates to a novel organic light emitting material and an organic light emitting device including the same.

### CROSS-REFERENCE TO RELATED APPLICATION(S)

This application claims the benefit of Korean Patent Applications No. 10-2021-0153380 filed on Nov. 9, 2021 and No. 10-2022-0139164 filed on Oct. 26, 2022 in the Korean Intellectual Property Office, the disclosures of which are incorporated herein by reference in their entirety.

### TECHNICAL FIELD

The present disclosure relates to a novel compound and an organic light emitting device including the same.

### BACKGROUND OF ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of new materials for the organic materials used in the organic light emitting devices as described above.

### PRIOR ART LITERATURE

### Patent Literature

(Patent Literature 1) Korean Unexamined Patent Publication No. 10-2000-0051826

### DETAILED DESCRIPTION OF THE INVENTION

### Technical Problem

The present disclosure relates to a novel organic light emitting material and an organic light emitting device including the same.

### Technical Solution

In the present disclosure, there is provided a compound represented by the following Chemical Formula 1:

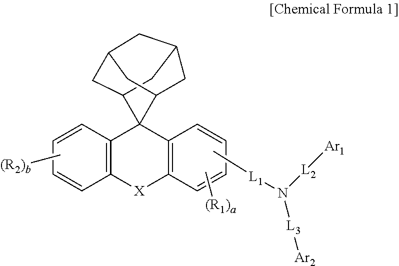

in Chemical Formula 1,
X is a single bond, O, or S,
R₁ and R₂ are each independently, hydrogen; deuterium; a halogen group; a nitrile group; a silyl group; a substituted or unsubstituted C₆₋₆₀ aryl; or a substituted or unsubstituted C₂₋₆₀ heteroaryl containing one or more selected from the group consisting of N, O, and S,
a is an integer of 0 to 3,
b is an integer of 0 to 4,
L₁ to L₃ are each independently a single bond; a substituted or unsubstituted C₆₋₆₀ arylene; or a substituted or unsubstituted C₂₋₆₀ heteroarylene containing one or more selected from the group consisting of N, O, and S, and
Ar₁ and Ar₂ are each independently a substituted or unsubstituted C₆₋₆₀ aryl; or a substituted or unsubstituted C₂₋₆₀ heteroaryl containing one or more selected from the group consisting of N, O, and S; wherein at least one of Ar₁ and Ar₂ is a substituent represented by Chemical Formula 2 below;

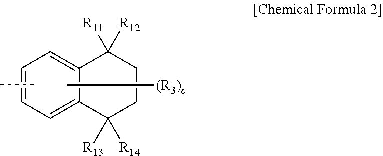

in Chemical Formula 2,
R₃ is deuterium; a halogen group; a nitrile group; a silyl group; a substituted or unsubstituted C₆₋₆₀ aryl; or a substituted or unsubstituted C₂₋₆₀ heteroaryl containing one or more selected from the group consisting of N, O, and S,
R₁₁ to R₁₄ are each independently —CH₃, —CH₂D, —CHD₂, or —CD₃,
c is an integer of 0 to 7, and
the silyl group is —Si(Z₁)(Z₂)(Z₃), wherein Z₁ to Z₃ are each independently a substituted or unsubstituted C₁₋₆₀ alkyl; or a substituted or unsubstituted C₆₋₆₀ aryl.

In addition, there is provided an organic light emitting device including: a first electrode; a second electrode that is provided opposite to the first electrode; and one or more organic material layers that are provided between the first electrode and the second electrode, wherein at least one layer of the organic material layers includes the compound represented by the Chemical Formula 1.

### Advantageous Effects

The compound represented by the Chemical Formula 1 may be used as a material for an organic material layer of an organic light emitting device, and may improve efficiency, low driving voltage, and/or lifespan of the organic light emitting device. In particular, the compound represented by the Chemical Formula 1 may be used as a material for hole injection, hole transport, light emission, electron transport, and/or electron injection.

### BRIEF DESCRIPTION OF THE DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a hole transport layer 6, an electron blocking layer 7, a light emitting layer 3, an electron injection and transport layer 8, and a cathode 4. In this structure, the compound represented by the Chemical Formula 1 may be included in the hole transport layer or the electron blocking layer.

### DETAILED DESCRIPTION OF THE EMBODIMENTS

Hereinafter, embodiments of the present disclosure will be described in more detail to facilitate understanding of the invention.

In the present disclosure, a compound represented by the Chemical Formula 1 above is provided.

As used herein, the notation [symbol] and [symbol] means a bond linked to another substituent group.

As used herein, the term “substituted or unsubstituted” means being unsubstituted or substituted with one or more substituents selected from the group consisting of deuterium; a halogen group; a nitrile group; a nitro group; a hydroxyl group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heteroaryl group containing at least one of N, O and S atoms, or being unsubstituted or substituted with a substituent in which two or more substituents of the above-exemplified substituents are connected. For example, “a substituent in which two or more substituents are connected” may be a biphenyl group. Namely, a biphenyl group may be an aryl group, or it may also be interpreted as a substituent in which two phenyl groups are connected.

In the present disclosure, the carbon number of a carbonyl group is not particularly limited, but is preferably 1 to 40. Specifically, the carbonyl group may be a substituent having the following structural formulae, but is not limited thereto.

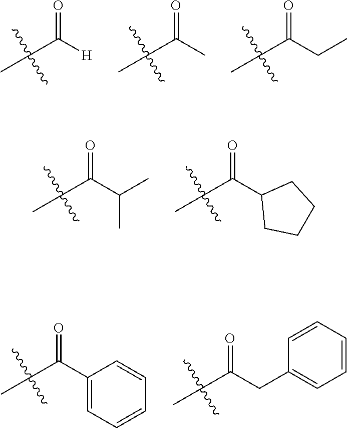

In the present disclosure, an ester group may have a structure in which oxygen of the ester group is substituted by a straight-chain, branched-chain, or cyclic alkyl group having 1 to 25 carbon atoms, or an aryl group having 6 to 25 carbon atoms. Specifically, the ester group may be a substituent having the following structural formulae, but is not limited thereto.

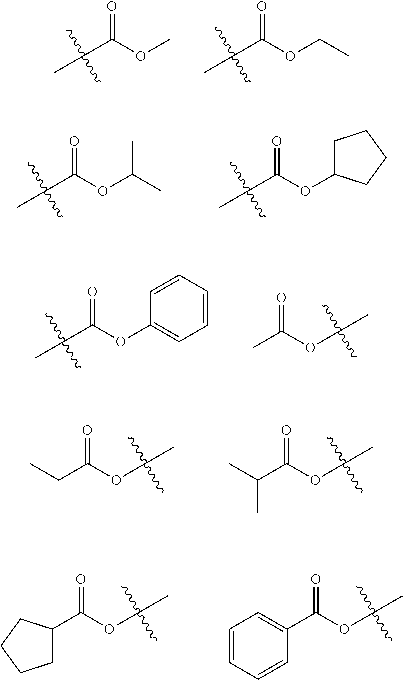

In the present disclosure, the carbon number of an imide group is not particularly limited, but is preferably 1 to 25. Specifically, the imide group may be a substituent having the following structural formulae, but is not limited thereto.

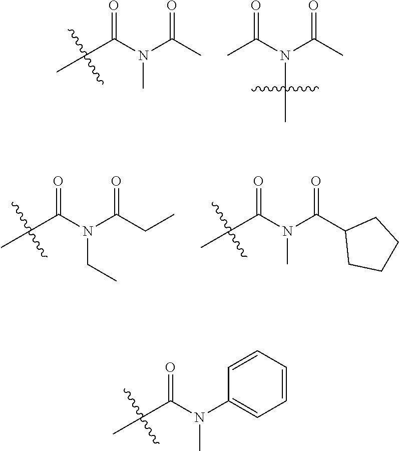

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, a phenylboron group, and the like, but is not limited thereto.

In the present disclosure, examples of a halogen group include fluorine, chlorine, bromine, or iodine.

In the present disclosure, the alkyl group may be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl) vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group and the like, but is not limited thereto.

In the present disclosure, a fluorenyl group may be substituted, and two substituents may be bonded to each other to form a spiro structure. In the case where the is fluorenyl group substituted,

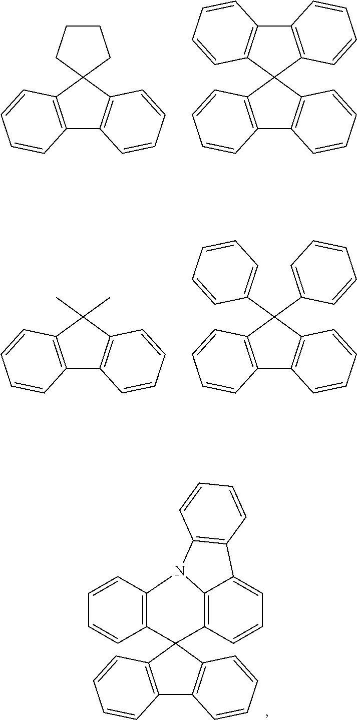

and the like can be formed. However, the structure is not limited thereto.

In the present disclosure, a heteroaryl group is a heteroaryl group containing at least one heteroatom of O, N, Si and S as a heterogeneous element, and the carbon number thereof is not particularly limited, but is preferably 2 to 60. According to one embodiment, the heteroaryl group has 6 to 30 carbon atoms. According to one embodiment, the heteroaryl group has 6 to 20 carbon atoms. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heteroaryl group. In the present disclosure, the alkenyl group in the aralkenyl group is the same as the aforementioned examples of the alkenyl group. In the present disclosure, the aforementioned description of the aryl group may be applied except that the arylene is a divalent group. In the present disclosure, the aforementioned description of the heteroaryl group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl group can be applied, except that the heteroaryl is not a monovalent group but formed by combining two substituent groups.

Preferably, R₁ and R₂ may each independently be hydrogen; deuterium; a substituted or unsubstituted C₆₋₂₀ aryl; or a substituted or unsubstituted C₂₋₂₀ heteroaryl containing one or more selected from the group consisting of N, O, and S.

More preferably, R₁ and R₂ may each independently be hydrogen, deuterium, phenyl, phenyl substituted with 1 or 2 tert-butyl, biphenylyl, naphthyl, or

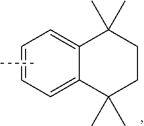

and when R₁ and R₂ are each phenyl, phenyl substituted with 1 or 2 tert-butyl, biphenylyl, naphthyl, or

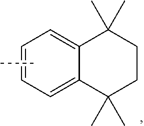

wherein the phenyl, phenyl substituted with 1 or 2 tert-butyl, biphenylyl, naphthyl, or

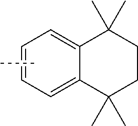

is unsubstituted or substituted with one or more deuterium.

Preferably, L₁ to L₃ may each independently be a single bond; a substituted or unsubstituted C₆₋₂₀ arylene; or a substituted or unsubstituted C₂₋₂₀ heteroarylene containing one or more selected from the group consisting of N, O, and S.

More preferably, L₁ to L₃ may each independently be a single bond, phenylene, or phenylene substituted with 4 deuterium.

Preferably, Ar₁ and Ar₂ may each independently be a substituted or unsubstituted C₆₋₂₀ aryl; or a substituted or unsubstituted C₂₋₂₀ heteroaryl containing one or more selected from the group consisting of N, O, and S; wherein at least one of Ar₁ and Ar₂ is a substituent represented by Chemical Formula 2 below;

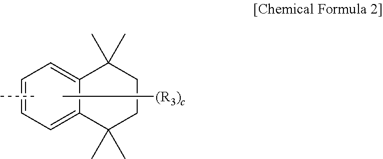

More preferably, in Ar₁ and Ar₂, R₃ of the Chemical Formula 2 may be hydrogen or deuterium, and c may be an integer of 0 to 7.

More preferably, Ar₁ and Ar₂ may each independently be phenyl, phenyl substituted with 1 tert-butyl, phenyl substituted with 1 adamantyl, biphenylyl, terphenylyl, naphthyl, phenyl naphthyl, naphthyl phenyl, dimethylfluorenyl, phenylfluorenyl, diphenylfluorenyl, carbazolyl, dibenzofuranyl, dibenzothiophenyl, or

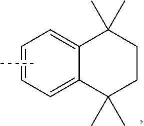

wherein Ar₁ and Ar₂ may be unsubstituted or substituted with one or more deuterium, and at least one of Ar₁ and Ar₂ may be

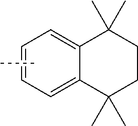

which is unsubstituted or substituted with one or more deuterium.

Most preferably, Ar₁ and Ar₂ may each independently be any one selected from the group consisting of the following, and at least one of Ar₁ and Ar₂ may be

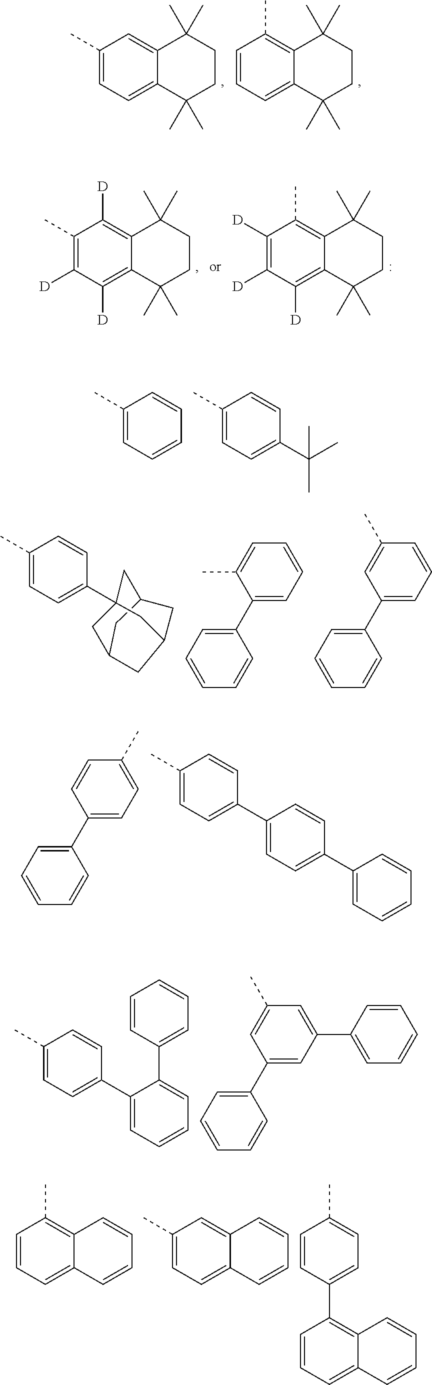

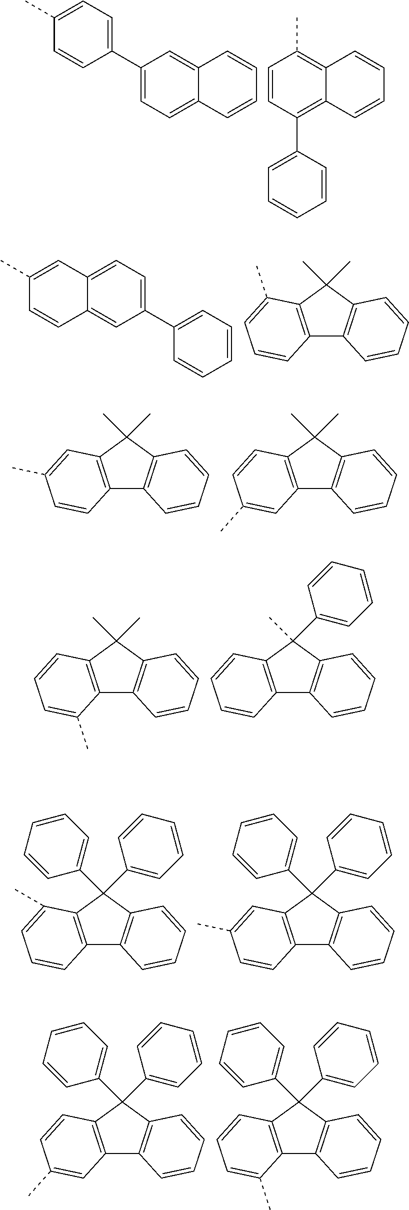

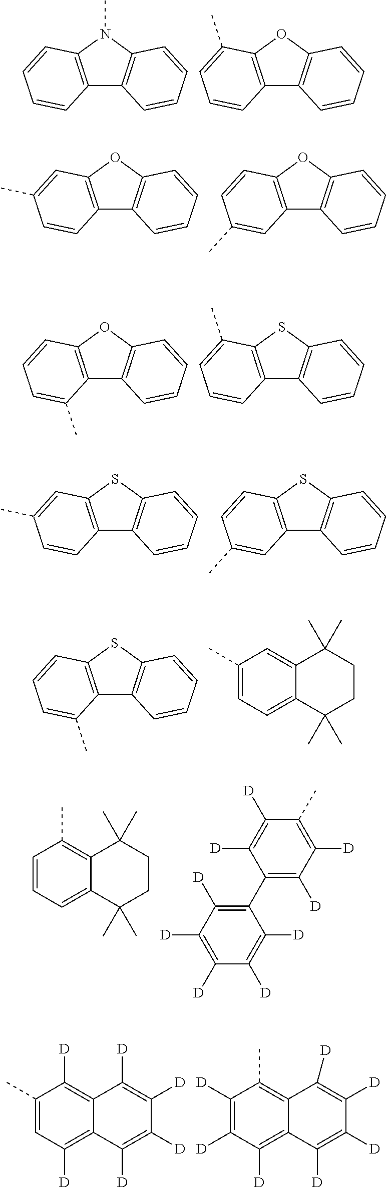

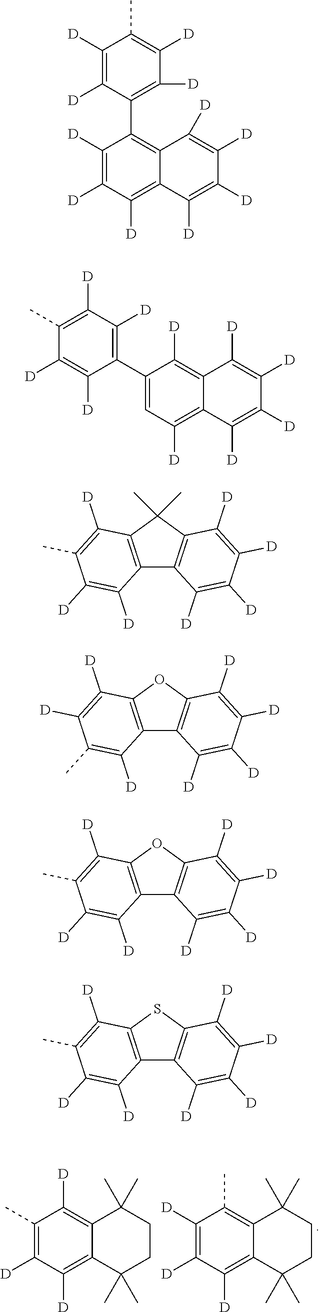

Preferably, the substituent represented by the Chemical Formula 2 may be any one selected from the group consisting of:

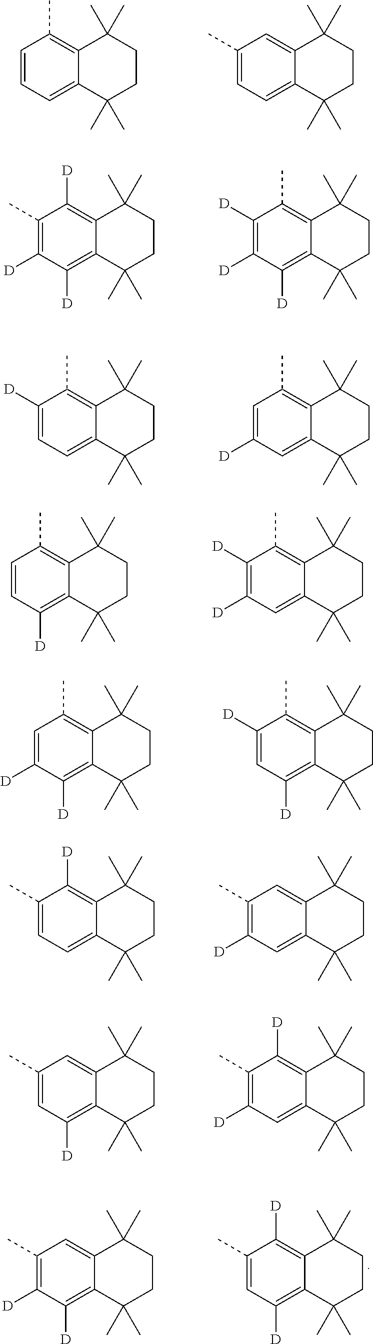

Preferably, R₁₁ to R₁₄ may each be —CH₃.

Representative examples of the compound represented by the Chemical Formula 1 are as follows:

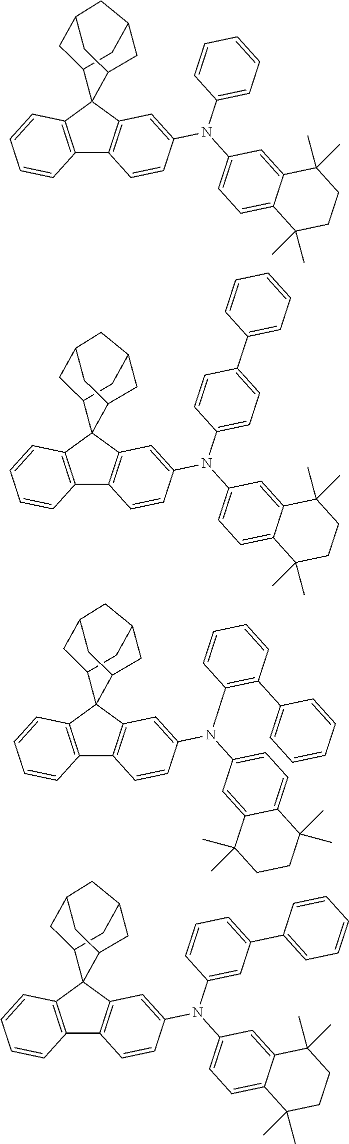

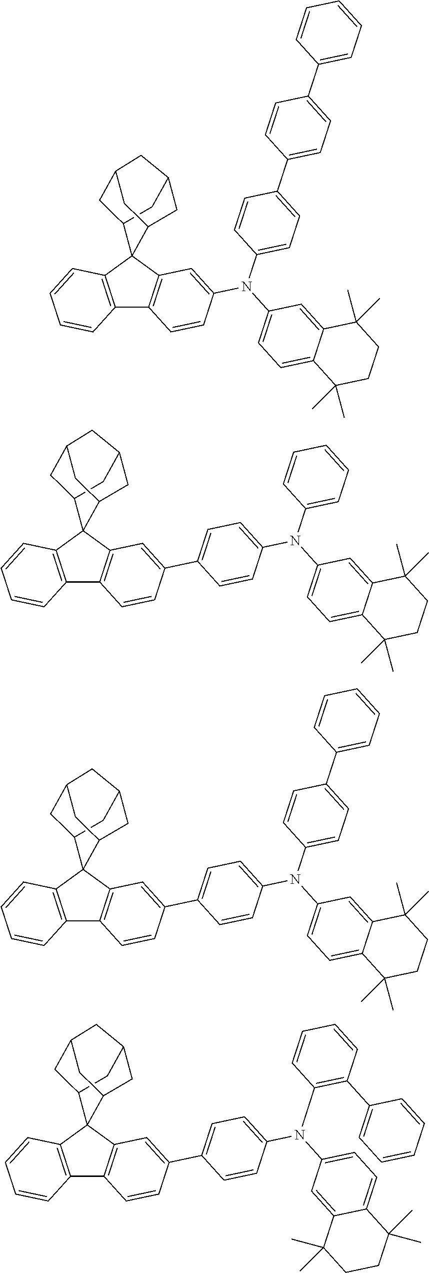

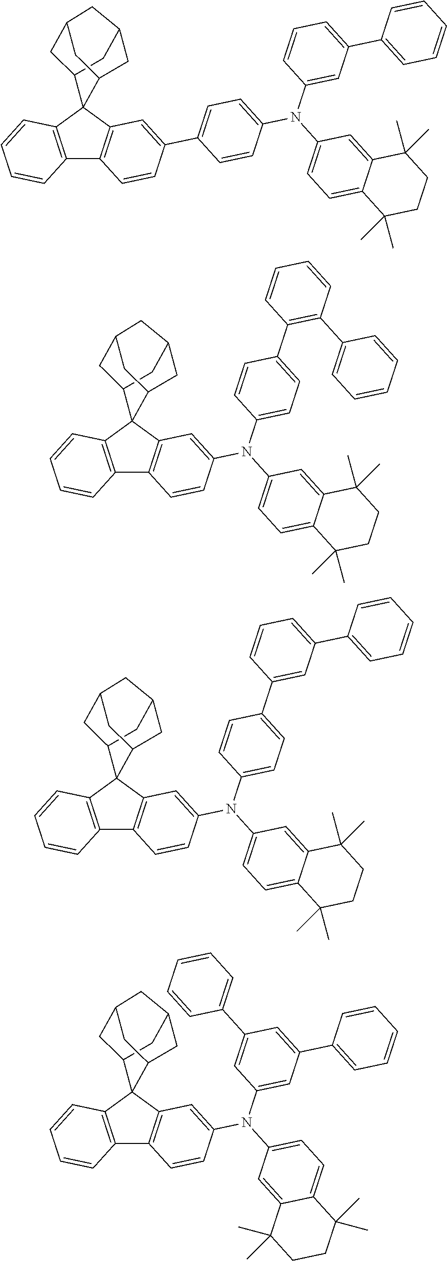

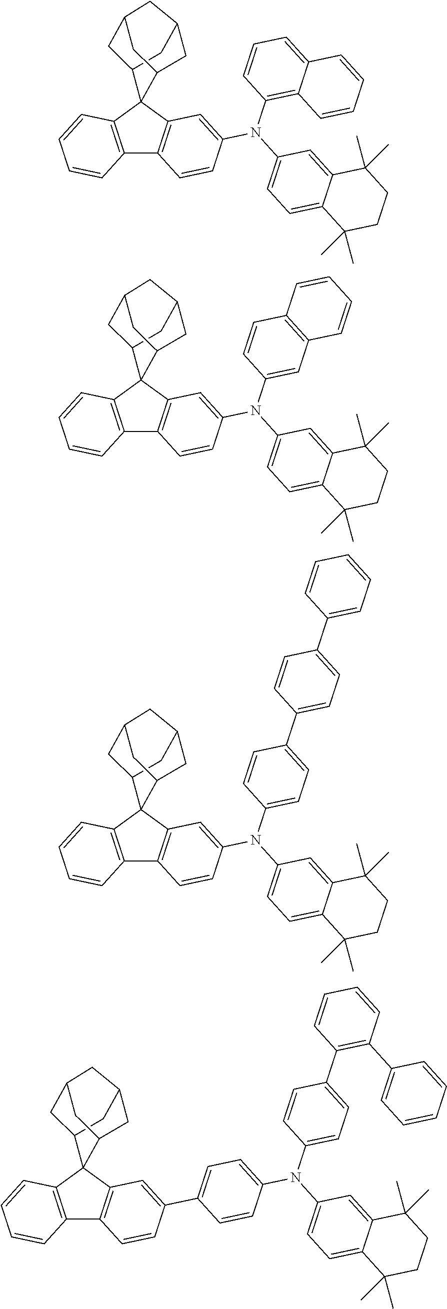

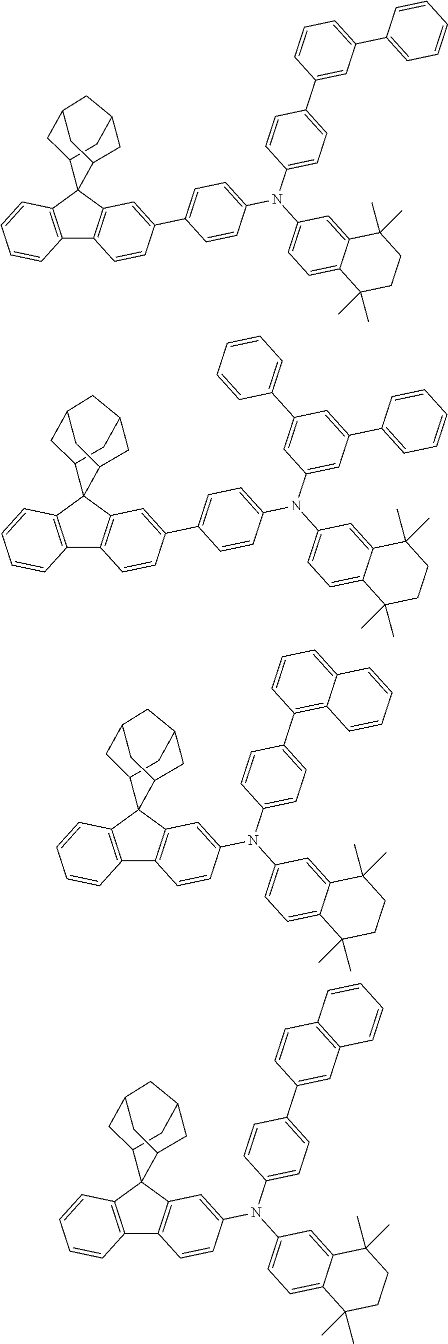

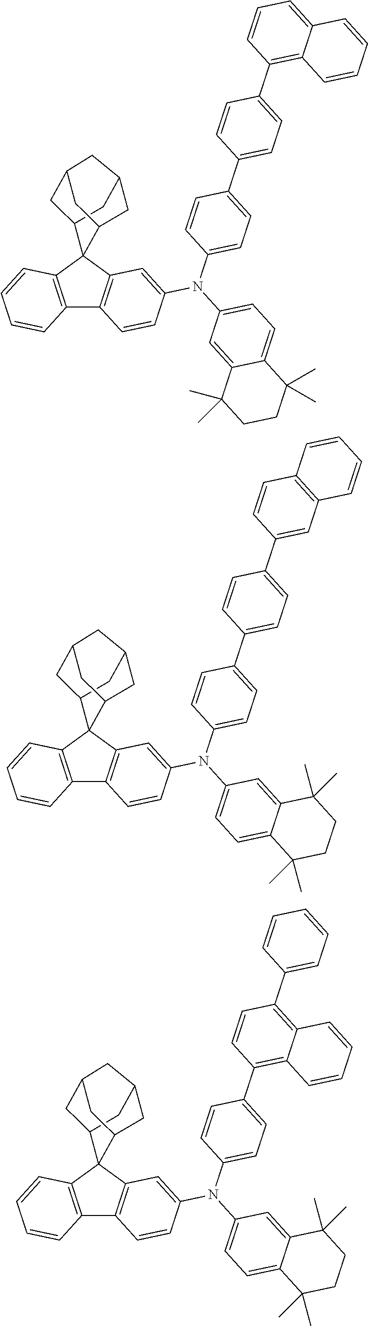

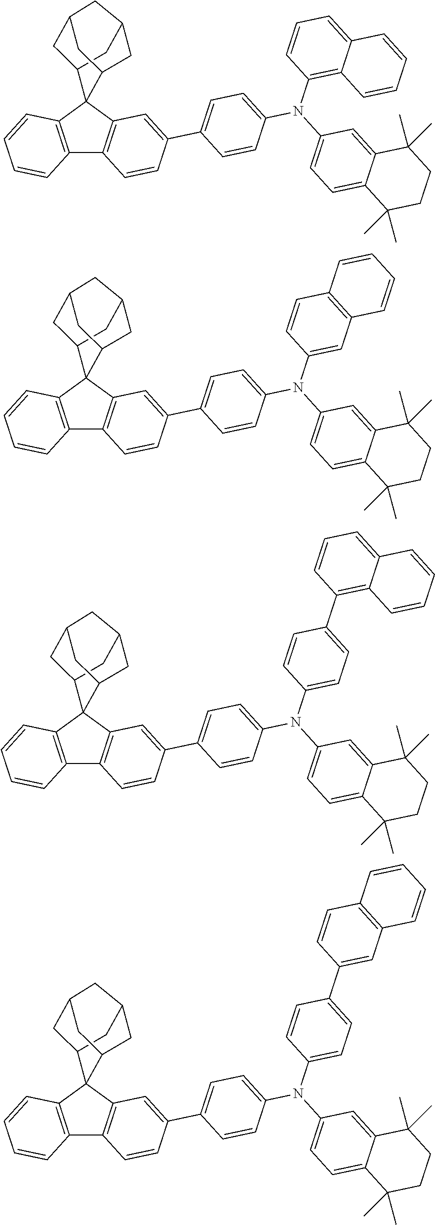

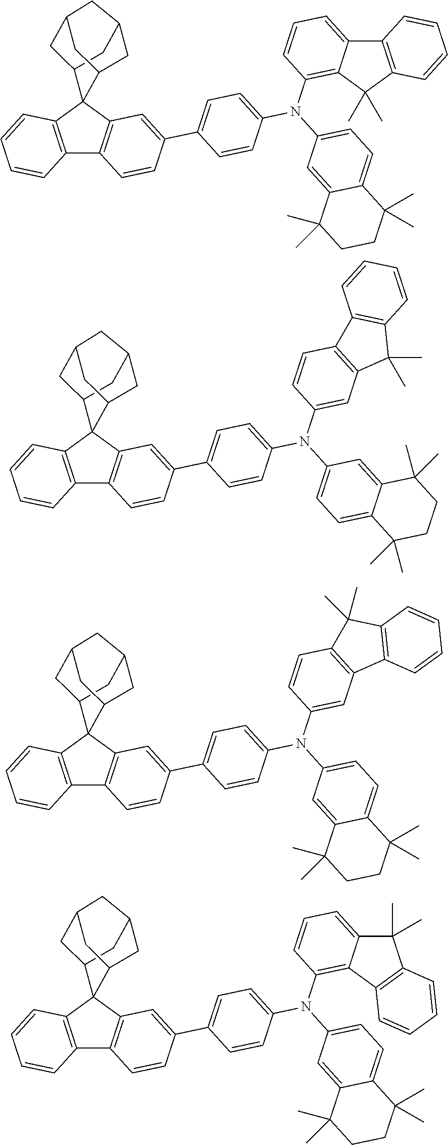

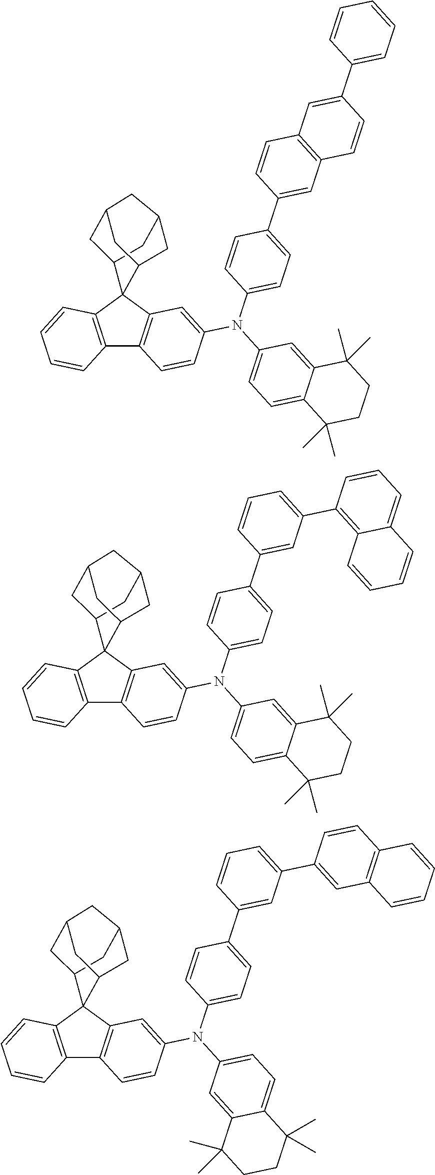

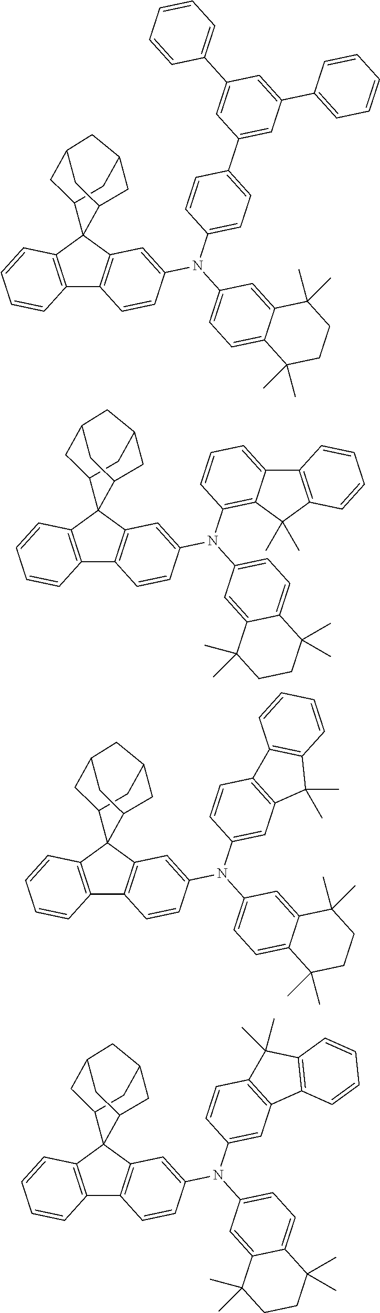

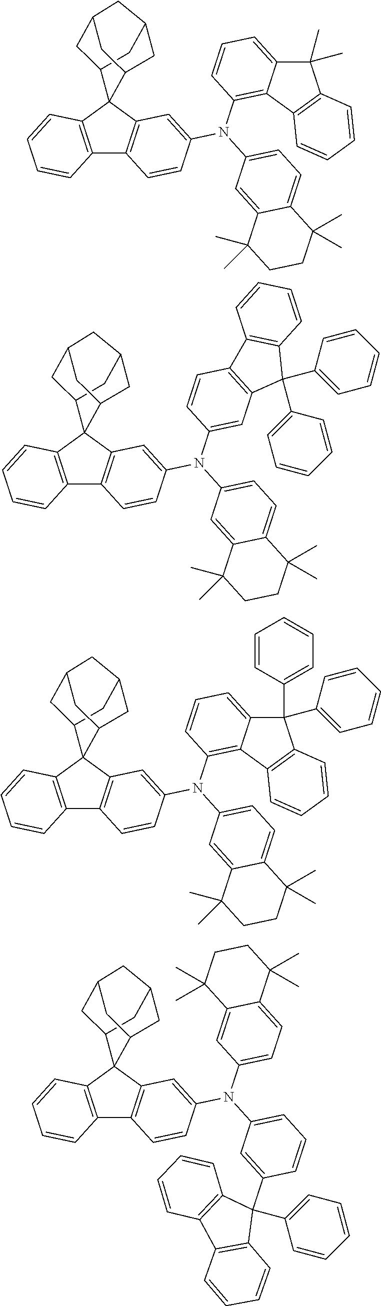

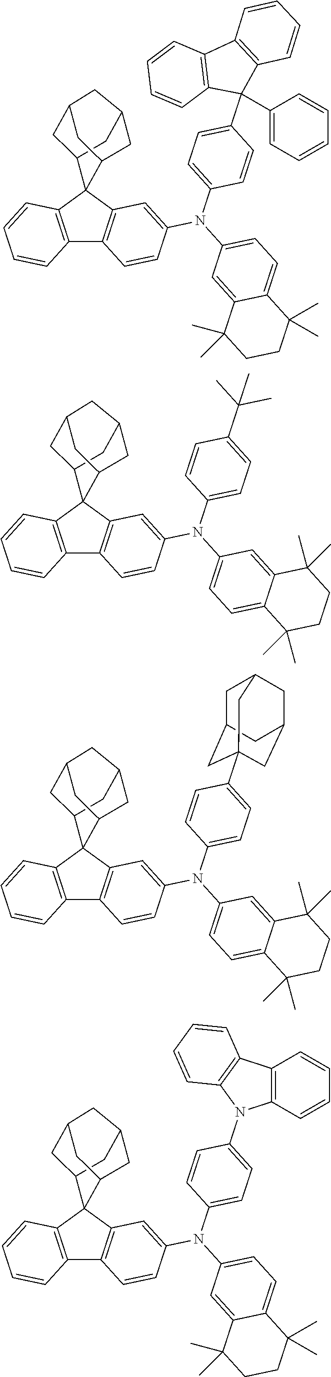

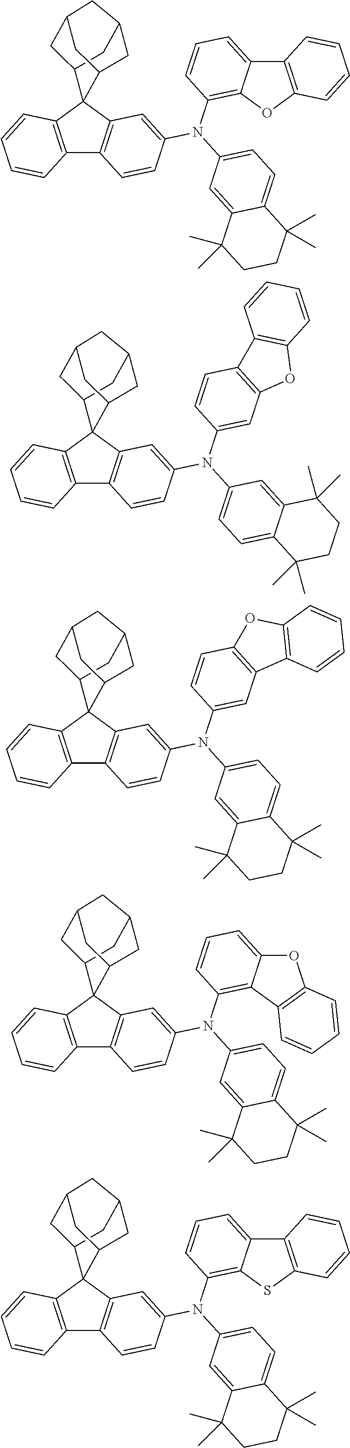

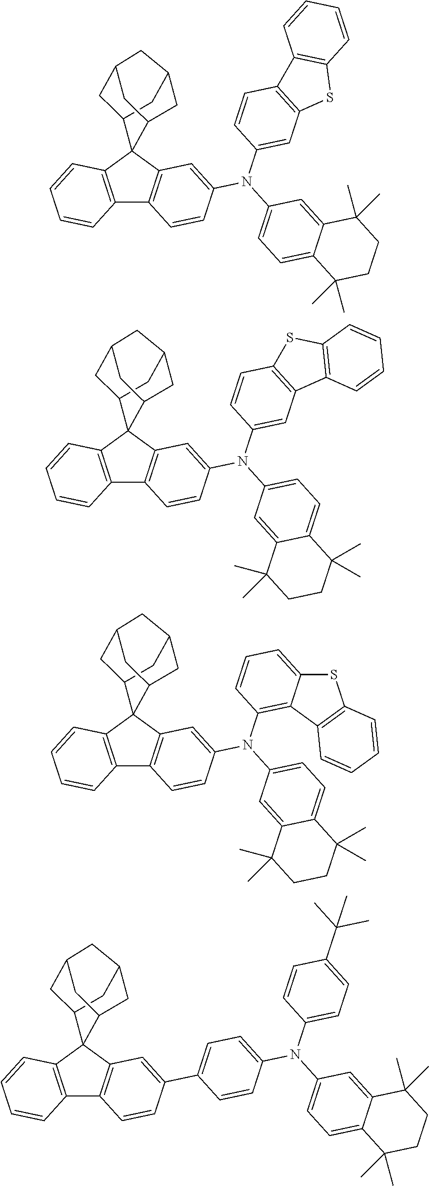

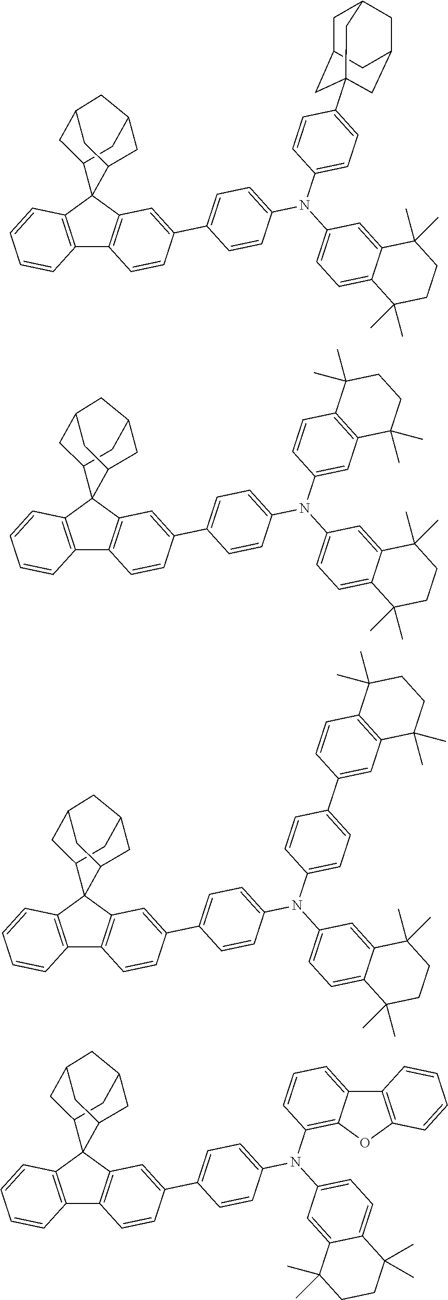

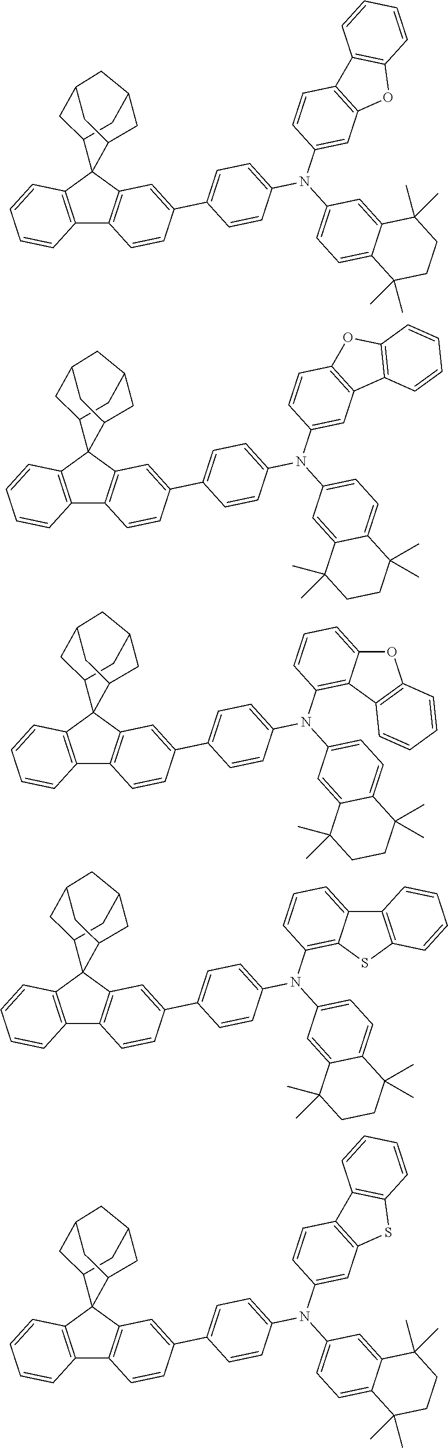

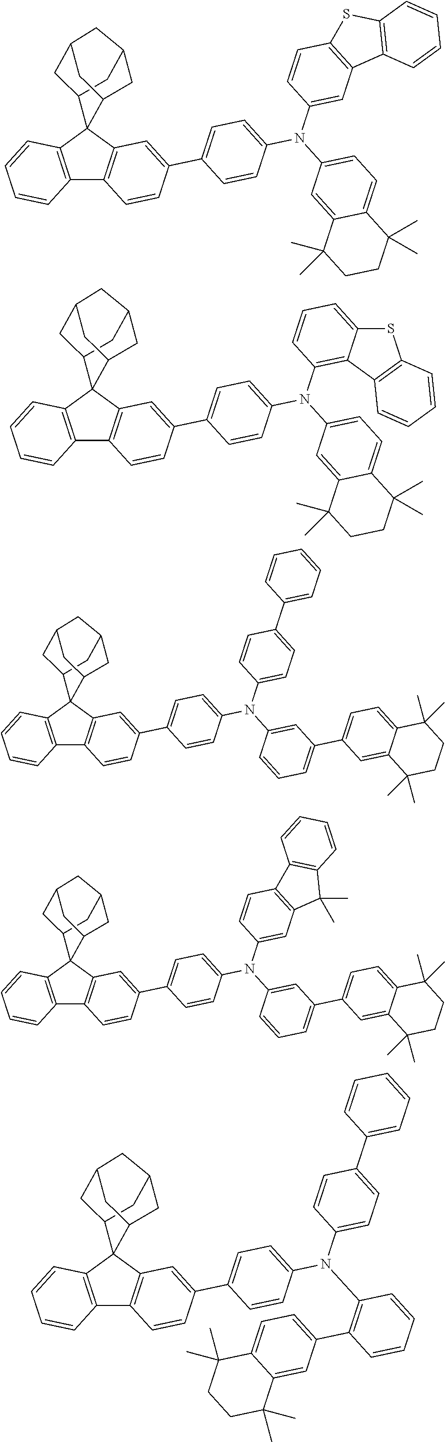

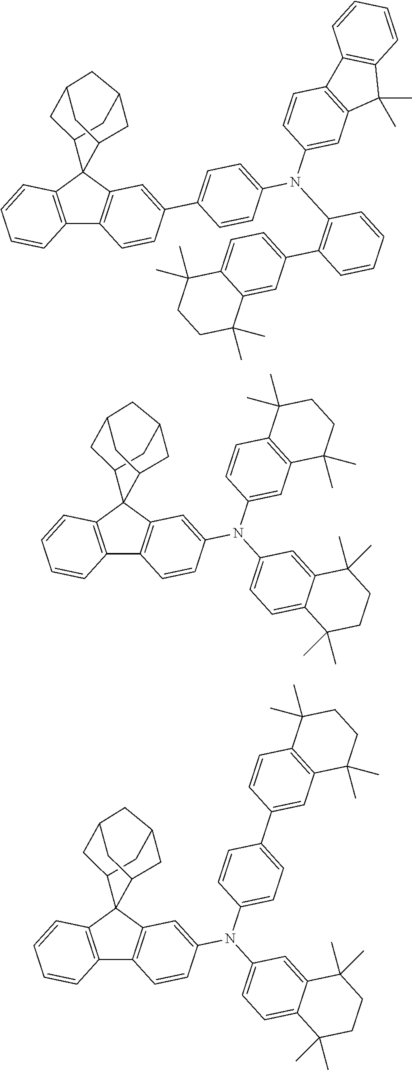

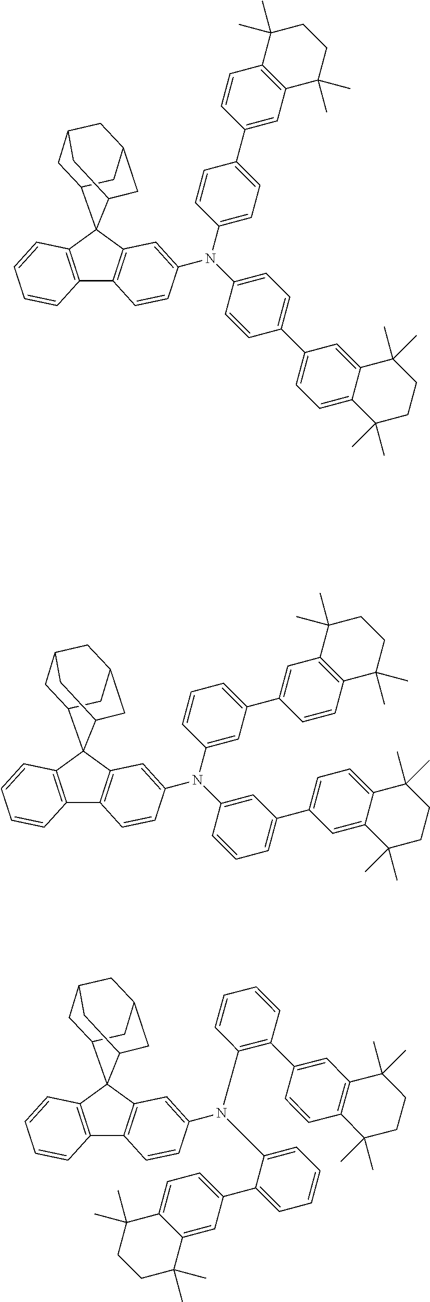 [SMILES: CC1(C)CCC(C)(C)c2cc(-c3ccc(N(c4ccc(-c5ccc6c(c5)C(C)(C)CCC6(C)C)cc4)c4ccc5c(c4)C4(c6ccccc6-5)C5CC6CC(C5)CC4C6)cc3)ccc21 ; CC1(C)CCC(C)(C)c2cc(-c3cccc(N(c4cccc(-c5ccc6c(c5)C(C)(C)CCC6(C)C)c4)c4ccc5c(c4)C4(c6ccccc6-5)C5CC6CC(C5)CC4C6)c3)ccc21 ; CC1(C)CCC(C)(C)c2cc(-c3ccccc3N(c3ccc4c(c3)C3(c5ccccc5-4)C4CC5CC(C4)CC3C5)c3ccccc3-c3ccc4c(c3)C(C)(C)CCC4(C)C)ccc21]

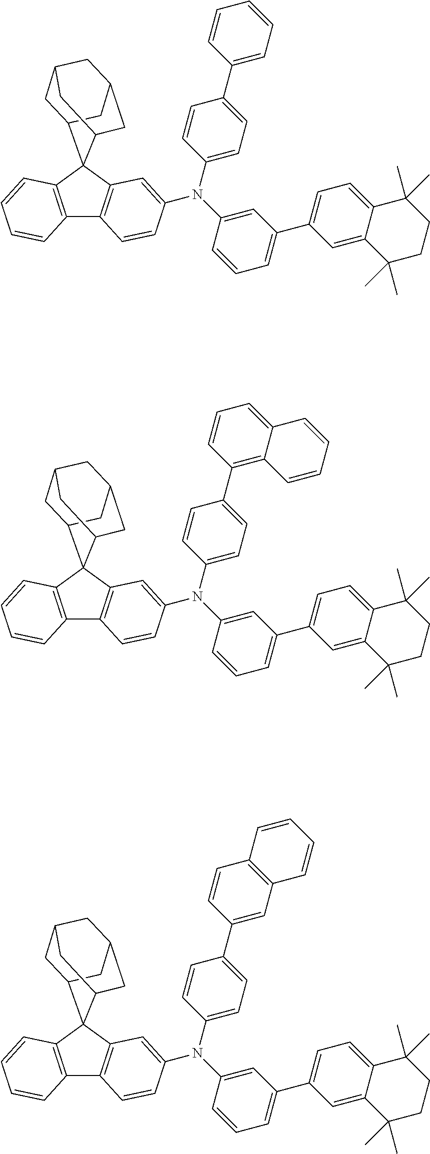 [SMILES: CC1(C)CCC(C)(C)c2cc(-c3cccc(N(c4ccc(-c5ccccc5)cc4)c4ccc5c(c4)C4(c6ccccc6-5)C5CC6CC(C5)CC4C6)c3)ccc21 ; CC1(C)CCC(C)(C)c2cc(-c3cccc(N(c4ccc(-c5cccc6ccccc56)cc4)c4ccc5c(c4)C4(c6ccccc6-5)C5CC6CC(C5)CC4C6)c3)ccc21 ; CC1(C)CCC(C)(C)c2cc(-c3cccc(N(c4ccc(-c5ccc6ccccc6c5)cc4)c4ccc5c(c4)C4(c6ccccc6-5)C5CC6CC(C5)CC4C6)c3)ccc21]

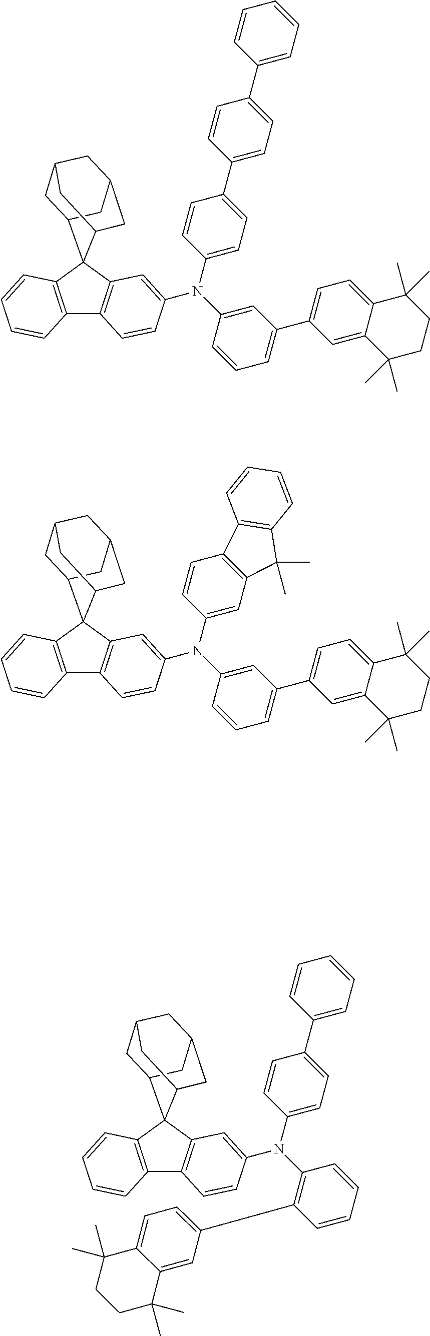

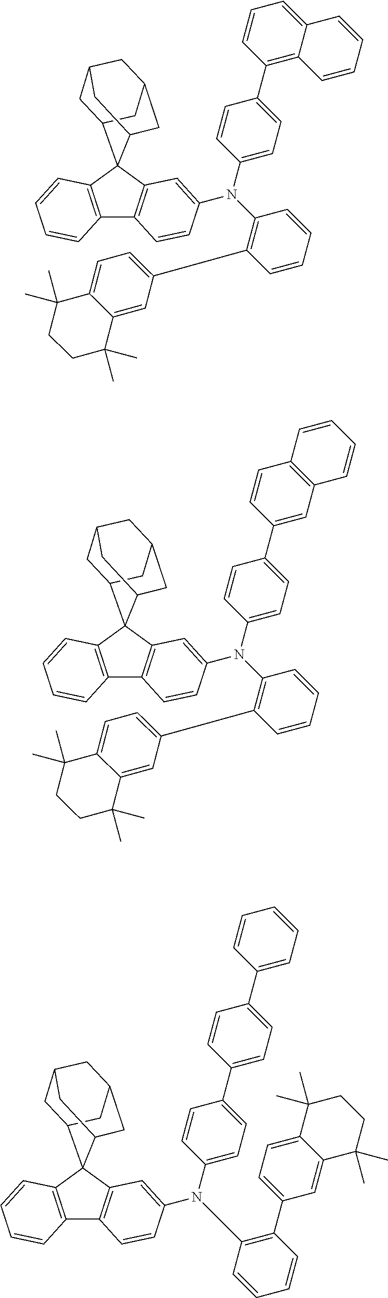 [SMILES: CC1(C)CCC(C)(C)c2cc(-c3ccccc3N(c3ccc(-c4cccc5ccccc45)cc3)c3ccc4c(c3)C3(c5ccccc5-4)C4CC5CC(C4)CC3C5)ccc21 ; CC1(C)CCC(C)(C)c2cc(-c3ccccc3N(c3ccc(-c4ccc5ccccc5c4)cc3)c3ccc4c(c3)C3(c5ccccc5-4)C4CC5CC(C4)CC3C5)ccc21 ; CC1(C)CCC(C)(C)c2cc(-c3ccccc3N(c3ccc(-c4ccc(-c5ccccc5)cc4)cc3)c3ccc4c(c3)C3(c5ccccc5-4)C4CC5CC(C4)CC3C5)ccc21]

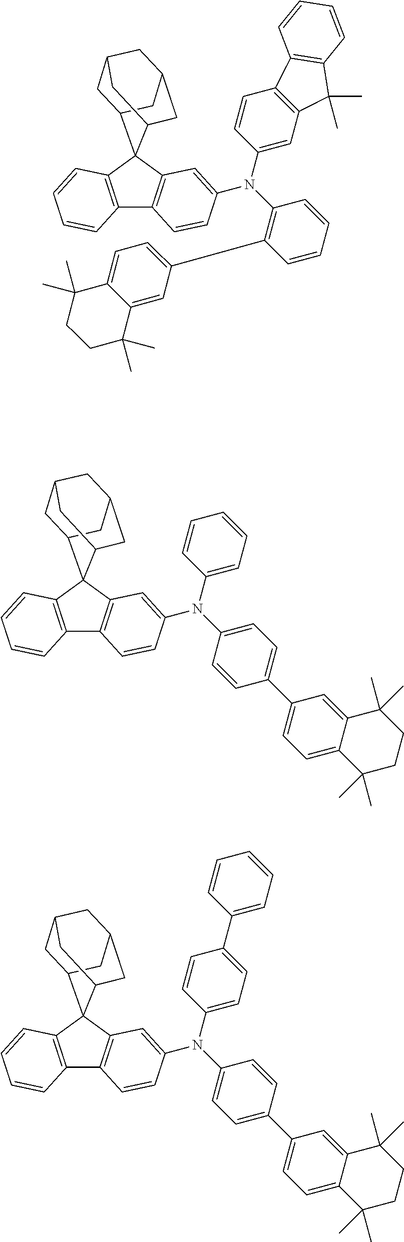

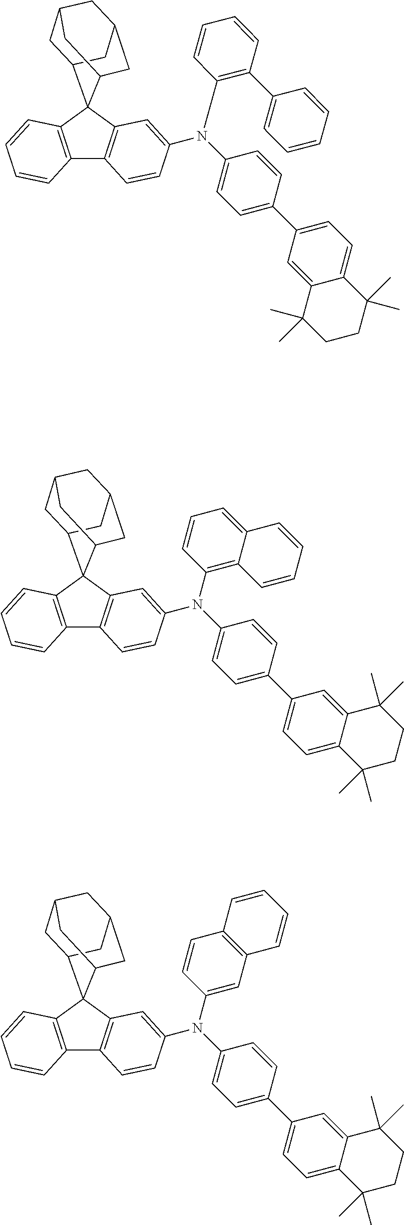 [SMILES: CC1(C)CCC(C)(C)c2cc(-c3ccc(N(c4ccc5c(c4)C4(c6ccccc6-5)C5CC6CC(C5)CC4C6)c4ccccc4-c4ccccc4)cc3)ccc21 ; CC1(C)CCC(C)(C)c2cc(-c3ccc(N(c4ccc5c(c4)C4(c6ccccc6-5)C5CC6CC(C5)CC4C6)c4cccc5ccccc45)cc3)ccc21 ; CC1(C)CCC(C)(C)c2cc(-c3ccc(N(c4ccc5c(c4)C4(c6ccccc6-5)C5CC6CC(C5)CC4C6)c4ccc5ccccc5c4)cc3)ccc21]

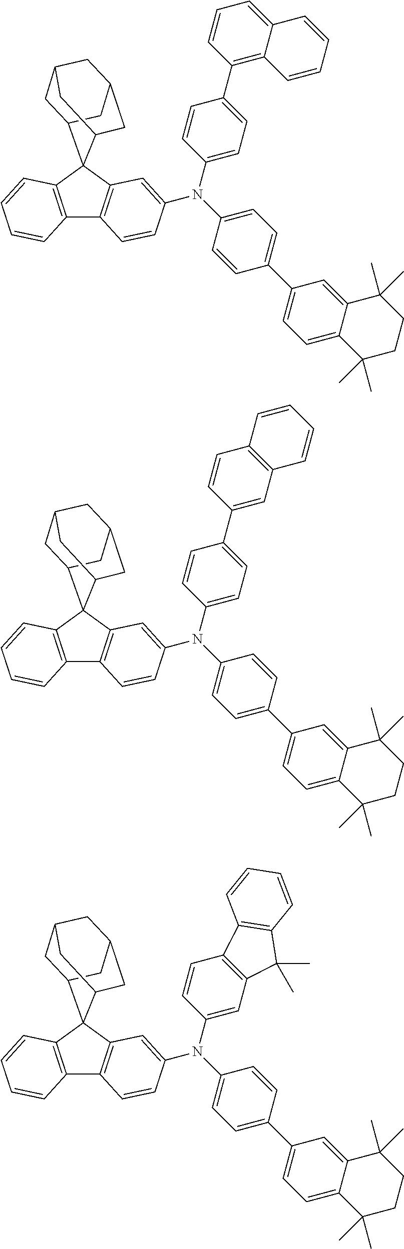 [SMILES: CC1(C)CCC(C)(C)c2cc(-c3ccc(N(c4ccc(-c5cccc6ccccc56)cc4)c4ccc5c(c4)C4(c6ccccc6-5)C5CC6CC(C5)CC4C6)cc3)ccc21 ; CC1(C)CCC(C)(C)c2cc(-c3ccc(N(c4ccc(-c5ccc6ccccc6c5)cc4)c4ccc5c(c4)C4(c6ccccc6-5)C5CC6CC(C5)CC4C6)cc3)ccc21 ; CC1(C)CCC(C)(C)c2cc(-c3ccc(N(c4ccc5c(c4)C(C)(C)c4ccccc4-5)c4ccc5c(c4)C4(c6ccccc6-5)C5CC6CC(C5)CC4C6)cc3)ccc21]

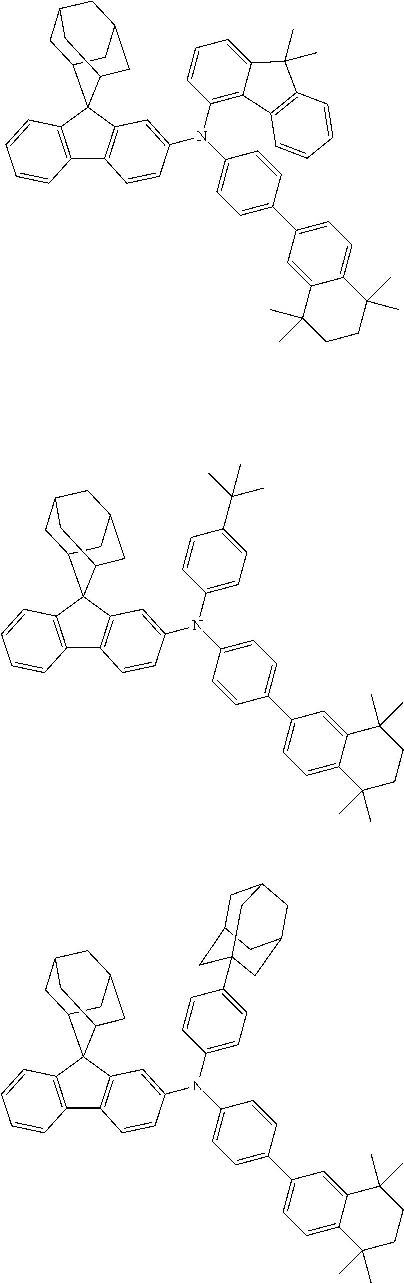 [SMILES: CC1(C)CCC(C)(C)c2cc(-c3ccc(N(c4ccc5c(c4)C4(c6ccccc6-5)C5CC6CC(C5)CC4C6)c4cccc5c4-c4ccccc4C5(C)C)cc3)ccc21 ; CC(C)(C)c1ccc(N(c2ccc(-c3ccc4c(c3)C(C)(C)CCC4(C)C)cc2)c2ccc3c(c2)C2(c4ccccc4-3)C3CC4CC(C3)CC2C4)cc1 ; CC1(C)CCC(C)(C)c2cc(-c3ccc(N(c4ccc(C56CC7CC(CC(C7)C5)C6)cc4)c4ccc5c(c4)C4(c6ccccc6-5)C5CC6CC(C5)CC4C6)cc3)ccc21]

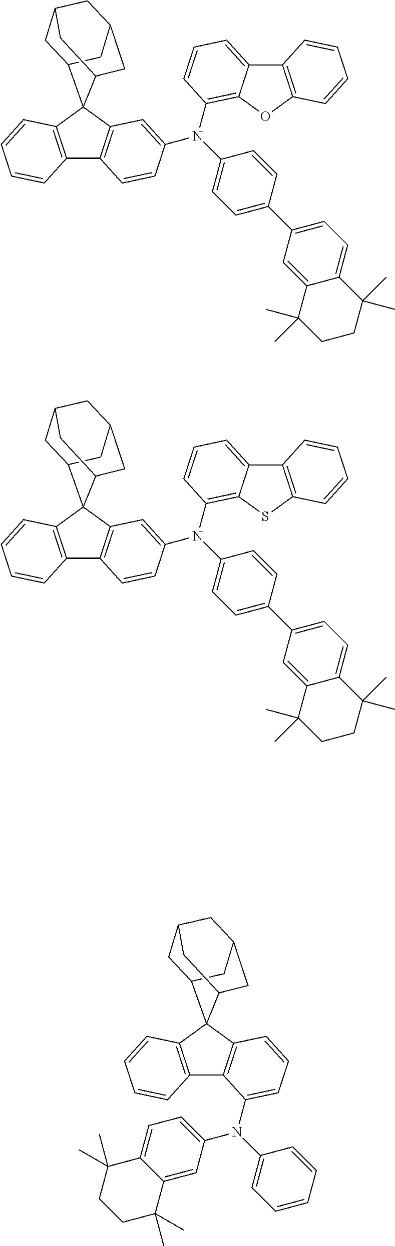

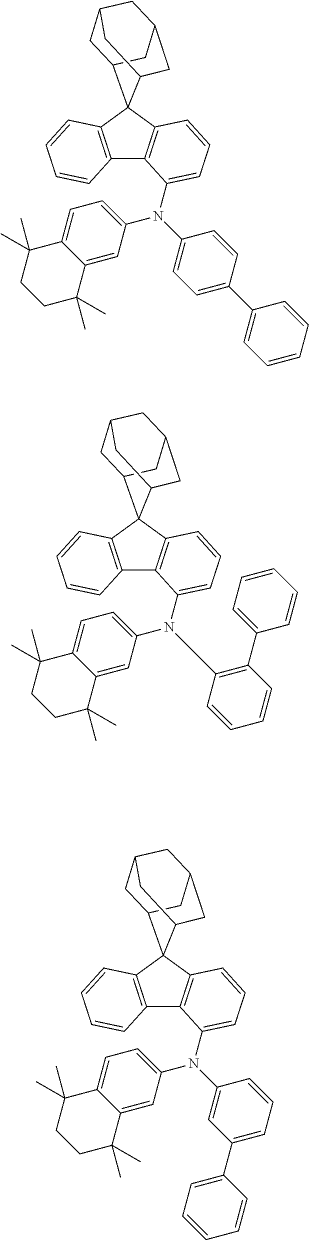 [SMILES: CC1(C)CCC(C)(C)c2cc(N(c3ccc(-c4ccccc4)cc3)c3cccc4c3-c3ccccc3C43C4CC5CC(C4)CC3C5)ccc21 ; CC1(C)CCC(C)(C)c2cc(N(c3ccccc3-c3ccccc3)c3cccc4c3-c3ccccc3C43C4CC5CC(C4)CC3C5)ccc21 ; CC1(C)CCC(C)(C)c2cc(N(c3cccc(-c4ccccc4)c3)c3cccc4c3-c3ccccc3C43C4CC5CC(C4)CC3C5)ccc21]

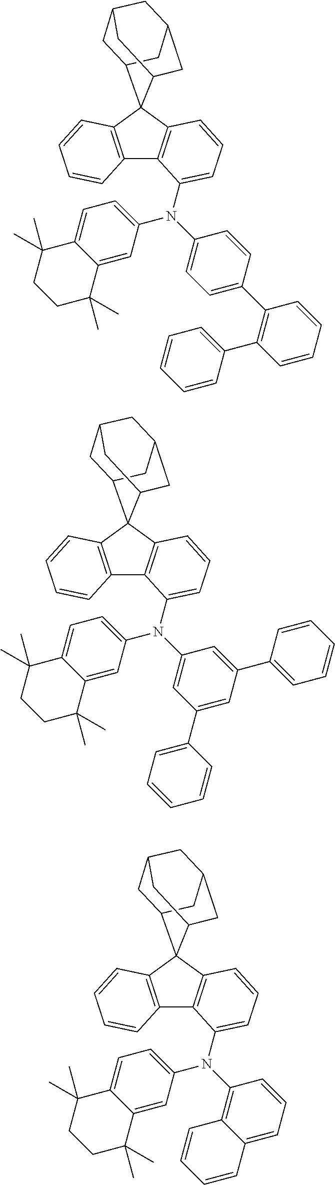

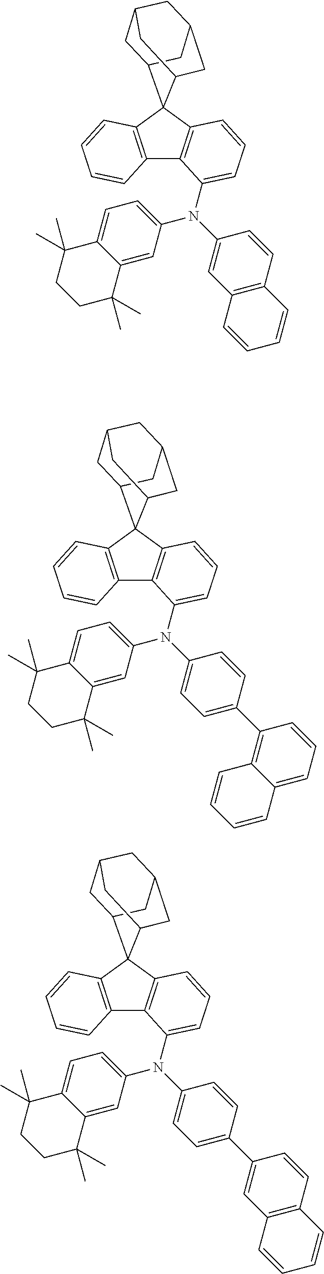 [SMILES: CC1(C)CCC(C)(C)c2cc(N(c3ccc4ccccc4c3)c3cccc4c3-c3ccccc3C43C4CC5CC(C4)CC3C5)ccc21 ; CC1(C)CCC(C)(C)c2cc(N(c3ccc(-c4cccc5ccccc45)cc3)c3cccc4c3-c3ccccc3C43C4CC5CC(C4)CC3C5)ccc21 ; CC1(C)CCC(C)(C)c2cc(N(c3ccc(-c4ccc5ccccc5c4)cc3)c3cccc4c3-c3ccccc3C43C4CC5CC(C4)CC3C5)ccc21]

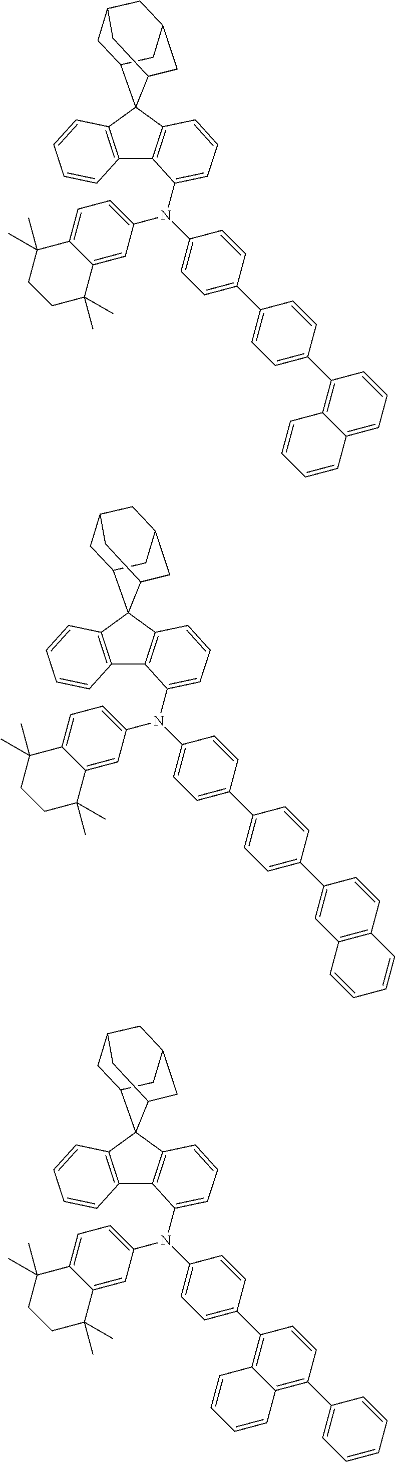 [SMILES: CC1(C)CCC(C)(C)c2cc(N(c3ccc(-c4ccc(-c5cccc6ccccc56)cc4)cc3)c3cccc4c3-c3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3cccc4c3-c3ccccc3C43C4CC5CC(C4)CC3C5)ccc21 ; CC1(C)CCC(C)(C)c2cc(N(c3ccc(-c4ccc(-c5ccccc5)c5ccccc45)cc3)c3cccc4c3-c3ccccc3C43C4CC5CC(C4)CC3C5)ccc21]

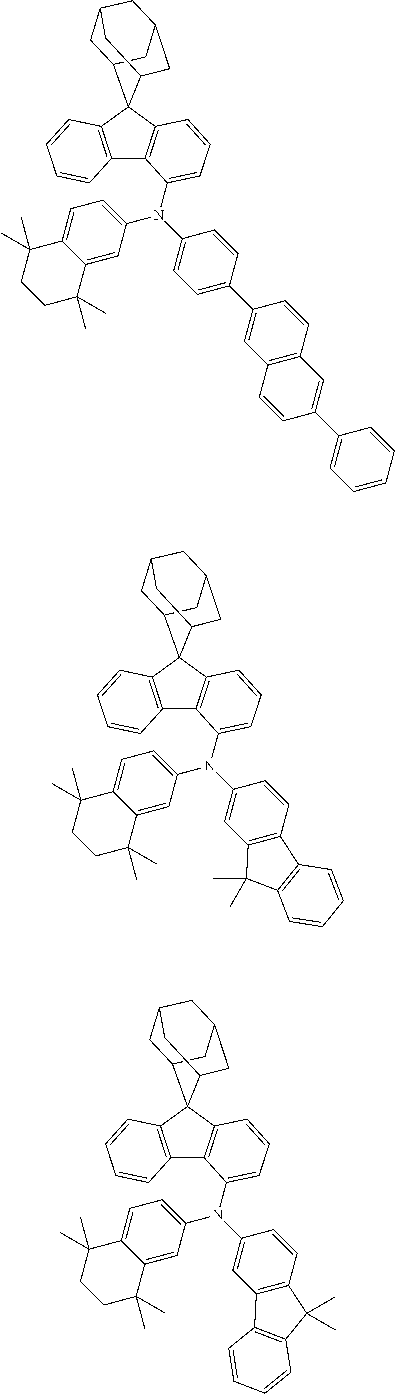

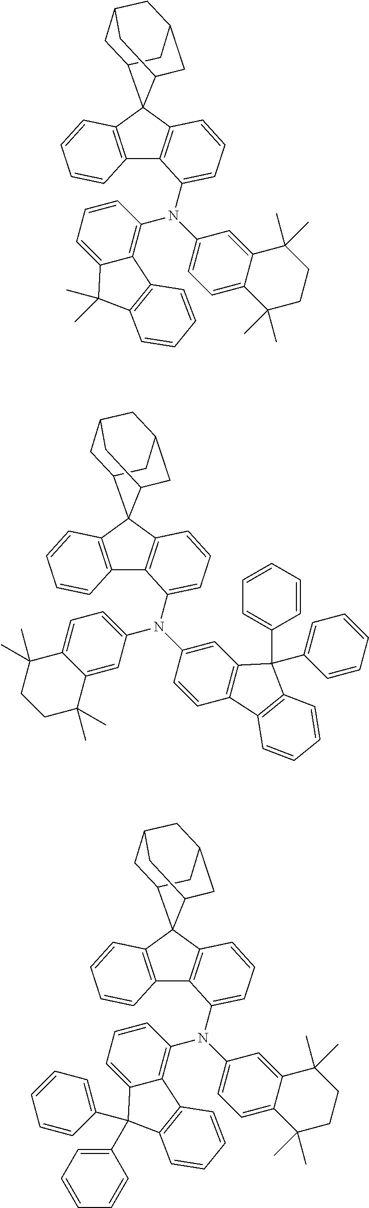

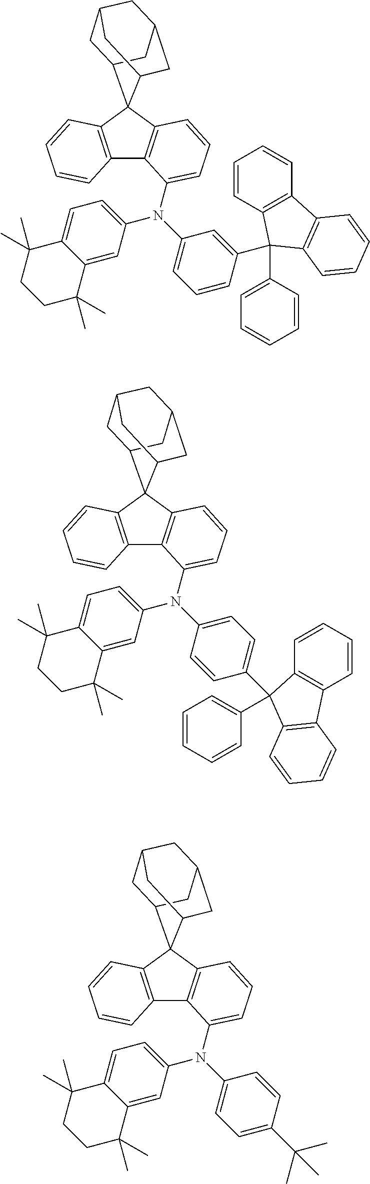 [SMILES: CC1(C)CCC(C)(C)c2cc(N(c3cccc(C4(c5ccccc5)c5ccccc5-c5ccccc54)c3)c3cccc4c3-c3ccccc3C43C4CC5CC(C4)CC3C5)ccc21 ; CC1(C)CCC(C)(C)c2cc(N(c3ccc(C4(c5ccccc5)c5ccccc5-c5ccccc54)cc3)c3cccc4c3-c3ccccc3C43C4CC5CC(C4)CC3C5)ccc21 ; CC(C)(C)c1ccc(N(c2ccc3c(c2)C(C)(C)CCC3(C)C)c2cccc3c2-c2ccccc2C32C3CC4CC(C3)CC2C4)cc1]

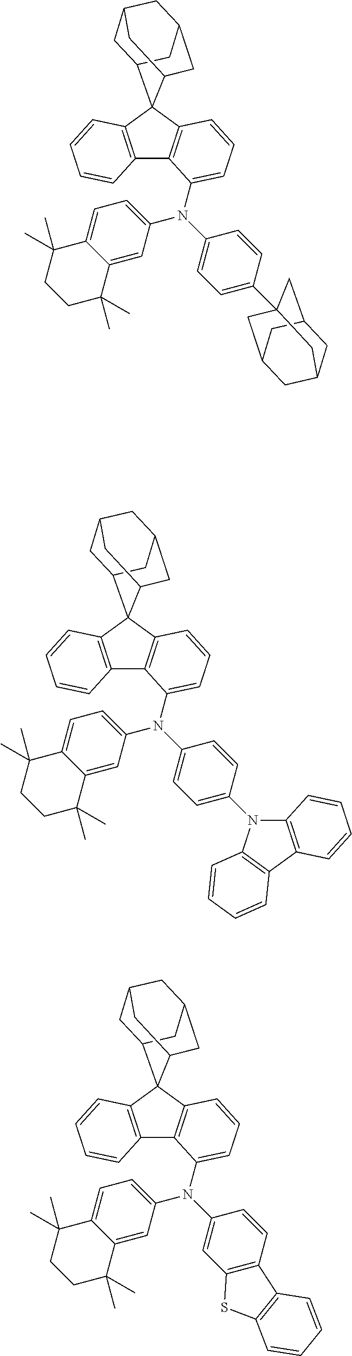 [SMILES: CC1(C)CCC(C)(C)c2cc(N(c3ccc(C45CC6CC(CC(C6)C4)C5)cc3)c3cccc4c3-c3ccccc3C43C4CC5CC(C4)CC3C5)ccc21 ; CC1(C)CCC(C)(C)c2cc(N(c3ccc(-n4c5ccccc5c5ccccc54)cc3)c3cccc4c3-c3ccccc3C43C4CC5CC(C4)CC3C5)ccc21 ; CC1(C)CCC(C)(C)c2cc(N(c3ccc4c(c3)sc3ccccc34)c3cccc4c3-c3ccccc3C43C4CC5CC(C4)CC3C5)ccc21]

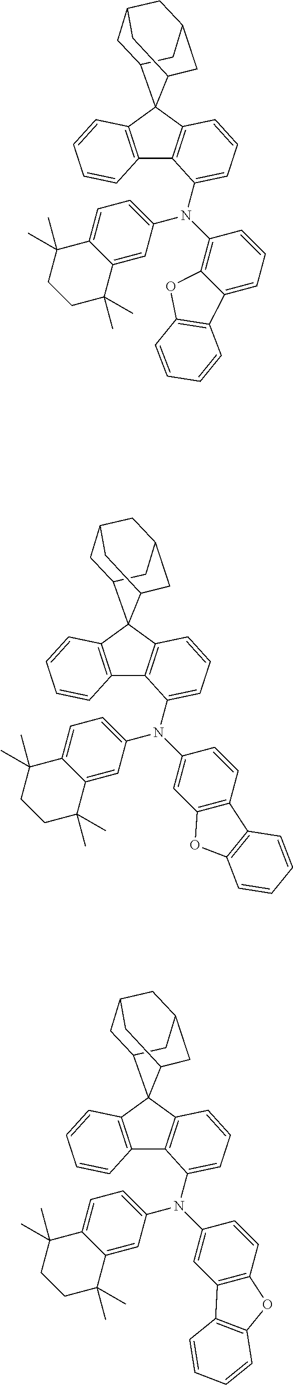 [SMILES: CC1(C)CCC(C)(C)c2cc(N(c3cccc4c3-c3ccccc3C43C4CC5CC(C4)CC3C5)c3cccc4c3oc3ccccc34)ccc21 ; CC1(C)CCC(C)(C)c2cc(N(c3ccc4c(c3)oc3ccccc34)c3cccc4c3-c3ccccc3C43C4CC5CC(C4)CC3C5)ccc21 ; CC1(C)CCC(C)(C)c2cc(N(c3ccc4oc5ccccc5c4c3)c3cccc4c3-c3ccccc3C43C4CC5CC(C4)CC3C5)ccc21]

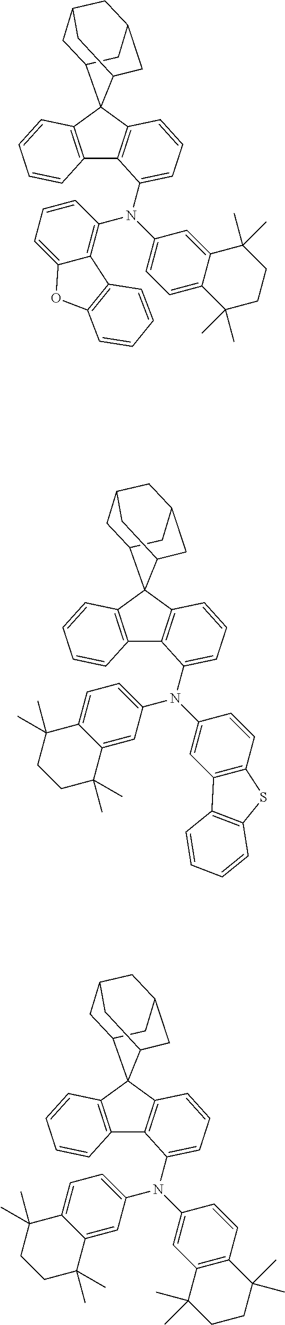

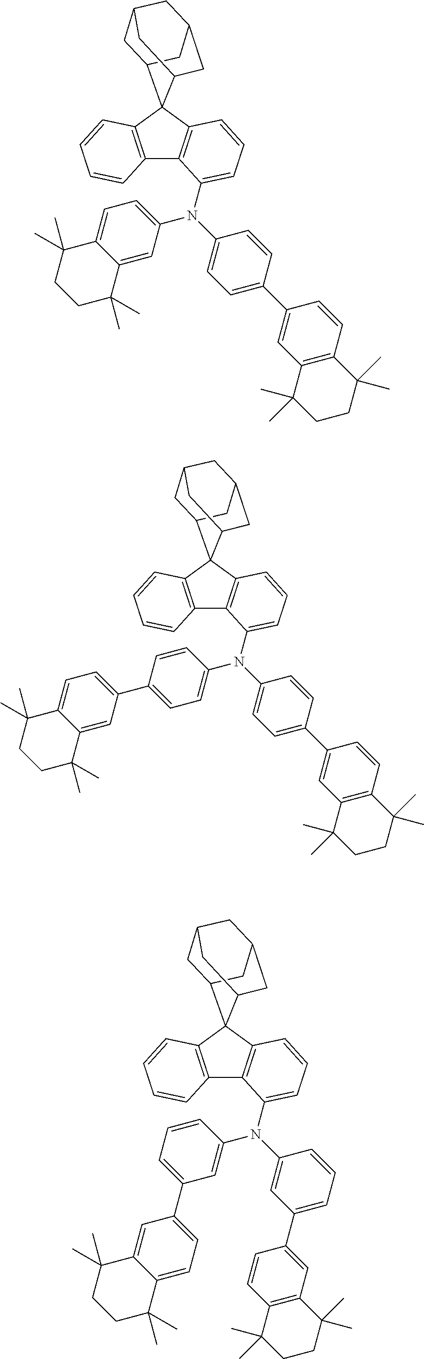

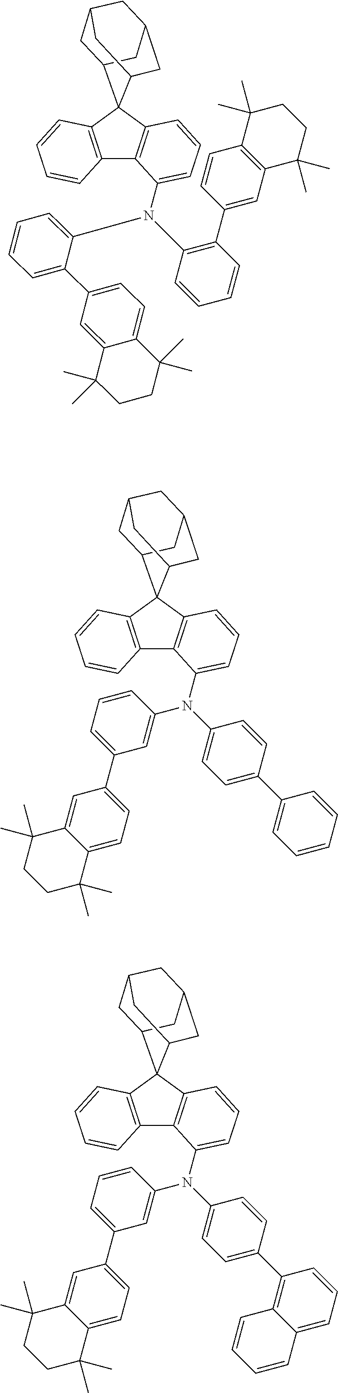

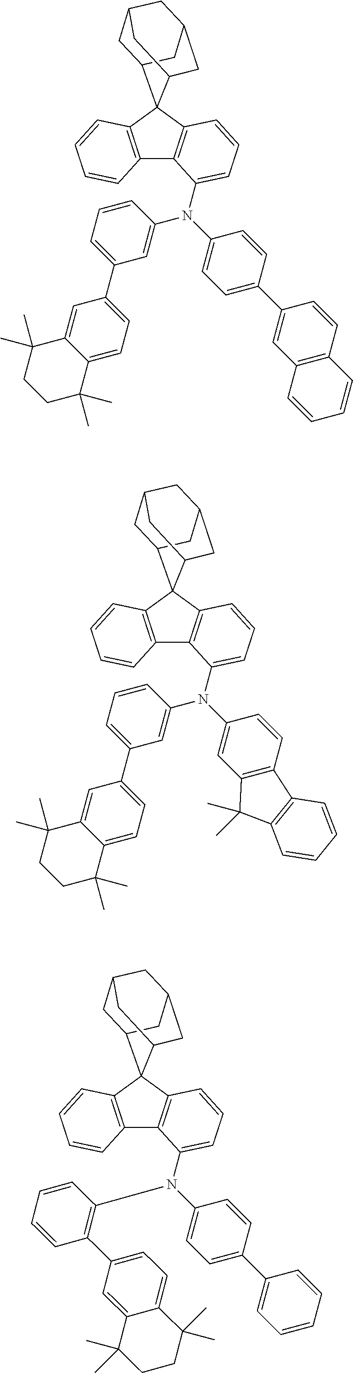 [SMILES: CC1(C)CCC(C)(C)c2cc(-c3cccc(N(c4ccc(-c5ccc6ccccc6c5)cc4)c4cccc5c4-c4ccccc4C54C5CC6CC(C5)CC4C6)c3)ccc21 ; CC1(C)CCC(C)(C)c2cc(-c3cccc(N(c4ccc5c(c4)C(C)(C)c4ccccc4-5)c4cccc5c4-c4ccccc4C54C5CC6CC(C5)CC4C6)c3)ccc21 ; CC1(C)CCC(C)(C)c2cc(-c3ccccc3N(c3ccc(-c4ccccc4)cc3)c3cccc4c3-c3ccccc3C43C4CC5CC(C4)CC3C5)ccc21]

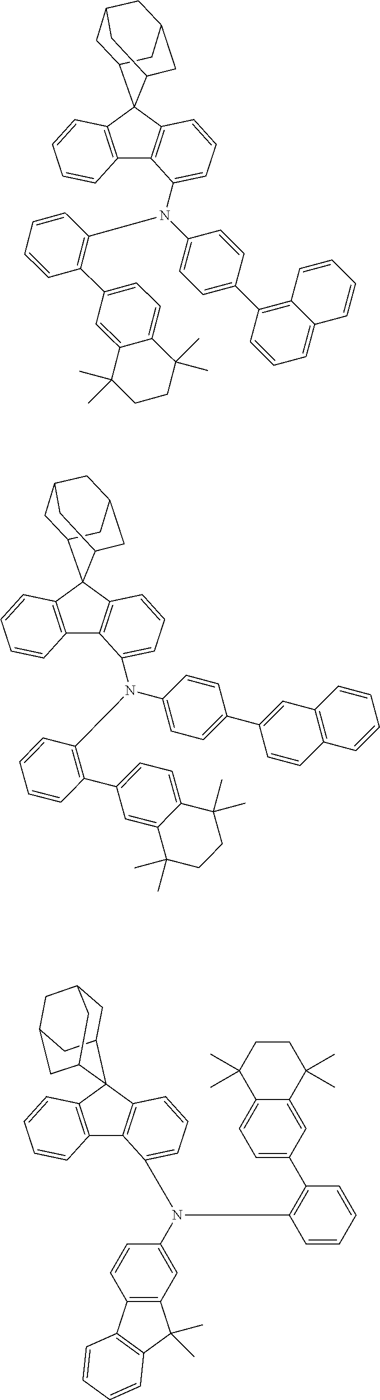 [SMILES: CC1(C)CCC(C)(C)c2cc(-c3ccccc3N(c3ccc(-c4cccc5ccccc45)cc3)c3cccc4c3-c3ccccc3C43C4CC5CC(C4)CC3C5)ccc21 ; CC1(C)CCC(C)(C)c2cc(-c3ccccc3N(c3ccc(-c4ccc5ccccc5c4)cc3)c3cccc4c3-c3ccccc3C43C4CC5CC(C4)CC3C5)ccc21 ; CC1(C)CCC(C)(C)c2cc(-c3ccccc3N(c3ccc4c(c3)C(C)(C)c3ccccc3-4)c3cccc4c3-c3ccccc3C43C4CC5CC(C4)CC3C5)ccc21]

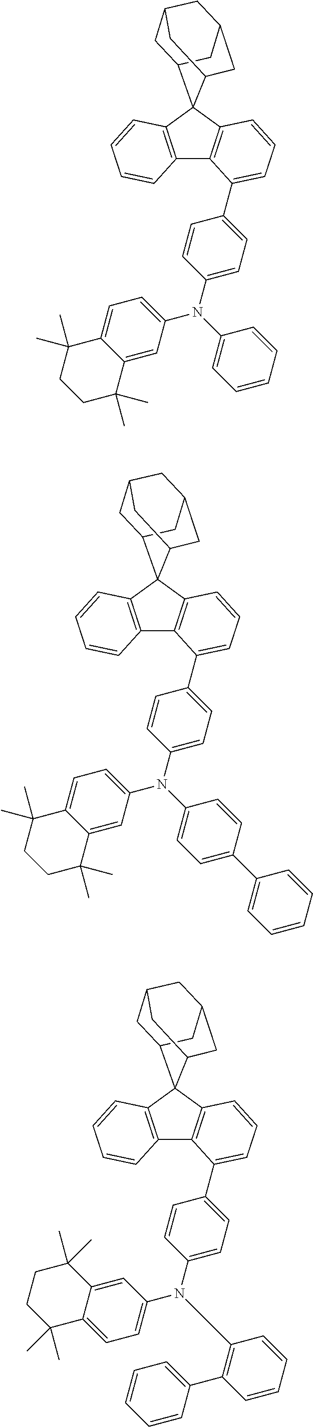 [SMILES: CC1(C)CCC(C)(C)c2cc(N(c3ccccc3)c3ccc(-c4cccc5c4-c4ccccc4C54C5CC6CC(C5)CC4C6)cc3)ccc21 ; CC1(C)CCC(C)(C)c2cc(N(c3ccc(-c4ccccc4)cc3)c3ccc(-c4cccc5c4-c4ccccc4C54C5CC6CC(C5)CC4C6)cc3)ccc21 ; CC1(C)CCC(C)(C)c2cc(N(c3ccc(-c4cccc5c4-c4ccccc4C54C5CC6CC(C5)CC4C6)cc3)c3ccccc3-c3ccccc3)ccc21]

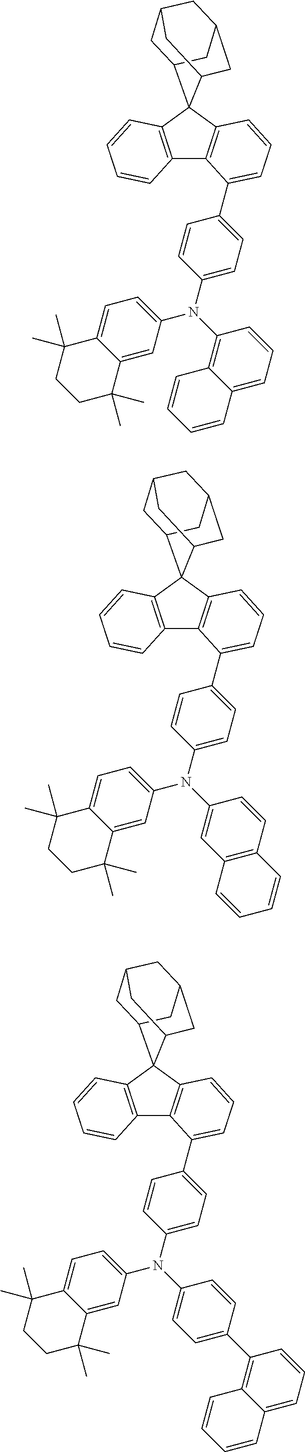

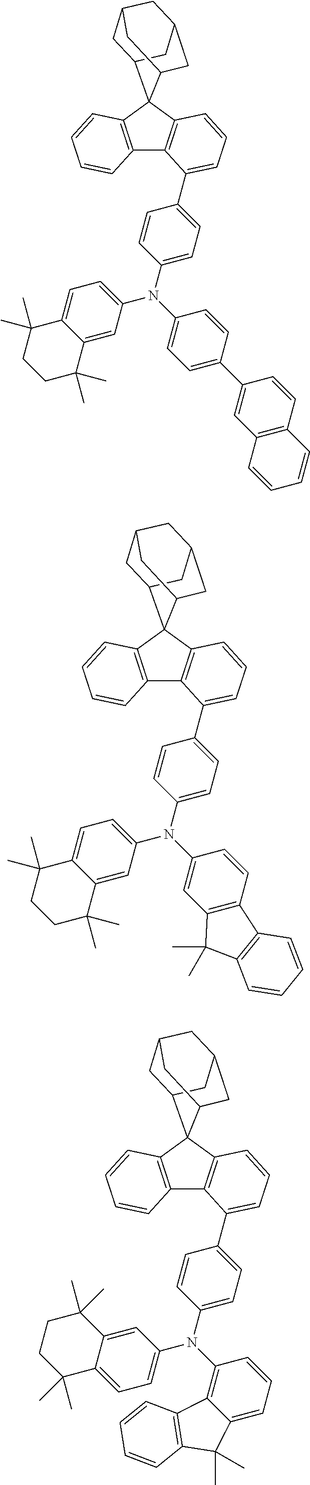 [SMILES: CC1(C)CCC(C)(C)c2cc(N(c3ccc(-c4ccc5ccccc5c4)cc3)c3ccc(-c4cccc5c4-c4ccccc4C54C5CC6CC(C5)CC4C6)cc3)ccc21 ; CC1(C)CCC(C)(C)c2cc(N(c3ccc(-c4cccc5c4-c4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4cccc5c4-c4ccccc4C54C5CC6CC(C5)CC4C6)cc3)c3cccc4c3-c3ccccc3C4(C)C)ccc21]

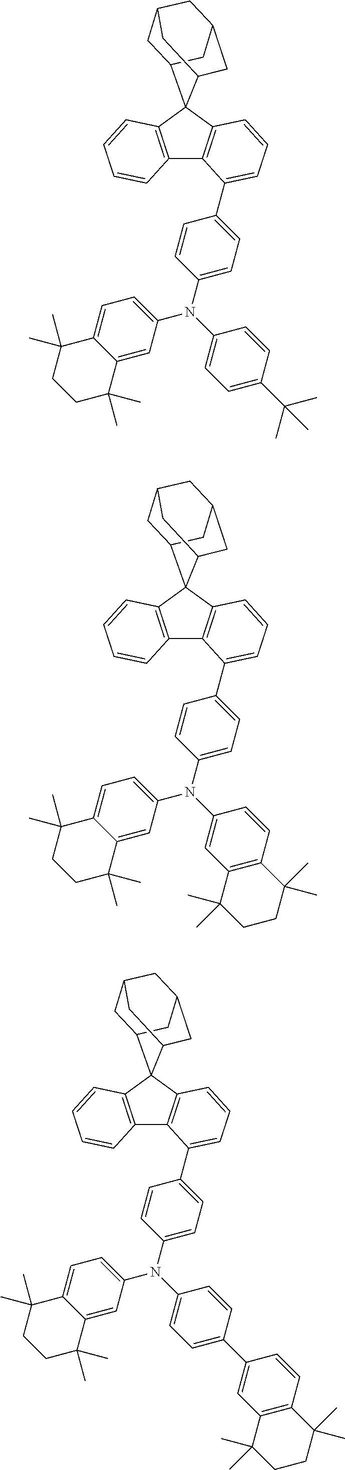

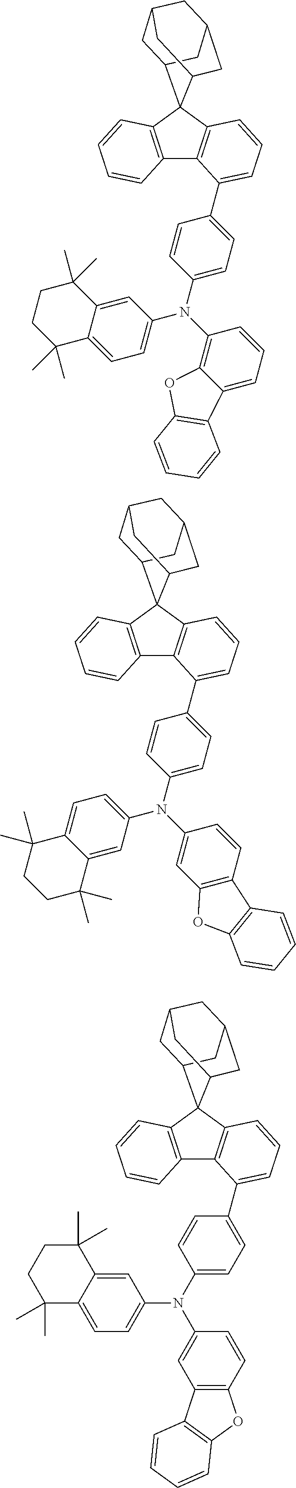

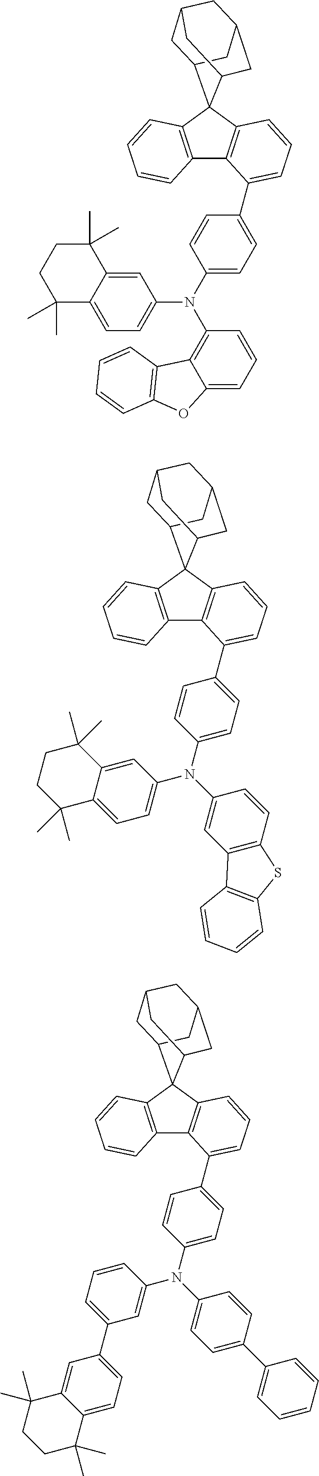 [SMILES: CC1(C)CCC(C)(C)c2cc(N(c3ccc(-c4cccc5c4-c4ccccc4C54C5CC6CC(C5)CC4C6)cc3)c3cccc4oc5ccccc5c34)ccc21 ; CC1(C)CCC(C)(C)c2cc(N(c3ccc(-c4cccc5c4-c4ccccc4C54C5CC6CC(C5)CC4C6)cc3)c3ccc4sc5ccccc5c4c3)ccc21 ; CC1(C)CCC(C)(C)c2cc(-c3cccc(N(c4ccc(-c5ccccc5)cc4)c4ccc(-c5cccc6c5-c5ccccc5C65C6CC7CC(C6)CC5C7)cc4)c3)ccc21]

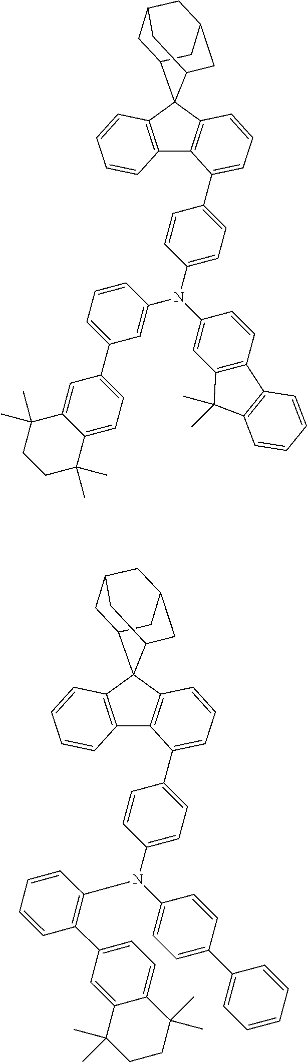 [SMILES: CC1(C)CCC(C)(C)c2cc(-c3cccc(N(c4ccc(-c5cccc6c5-c5ccccc5C65C6CC7CC(C6)CC5C7)cc4)c4ccc5c(c4)C(C)(C)c4ccccc4-5)c3)ccc21 ; CC1(C)CCC(C)(C)c2cc(-c3ccccc3N(c3ccc(-c4ccccc4)cc3)c3ccc(-c4cccc5c4-c4ccccc4C54C5CC6CC(C5)CC4C6)cc3)ccc21]

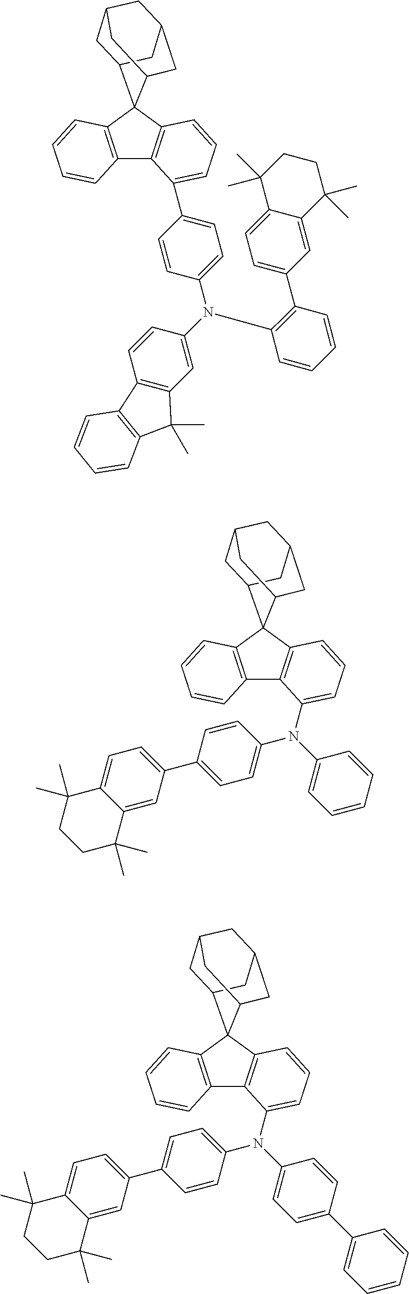

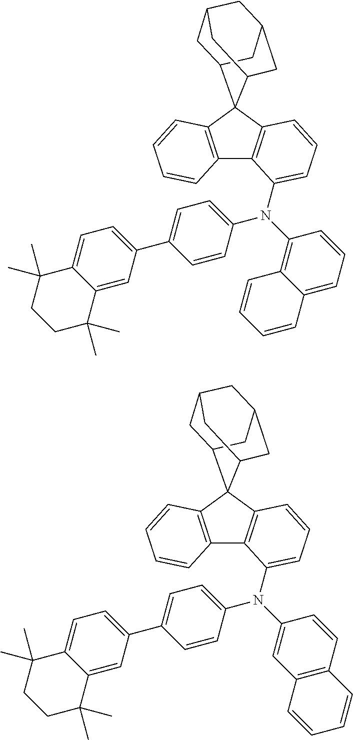 [SMILES: CC1(C)CCC(C)(C)c2cc(-c3ccc(N(c4cccc5c4-c4ccccc4C54C5CC6CC(C5)CC4C6)c4cccc5ccccc45)cc3)ccc21 ; CC1(C)CCC(C)(C)c2cc(-c3ccc(N(c4ccc5ccccc5c4)c4cccc5c4-c4ccccc4C54C5CC6CC(C5)CC4C6)cc3)ccc21]

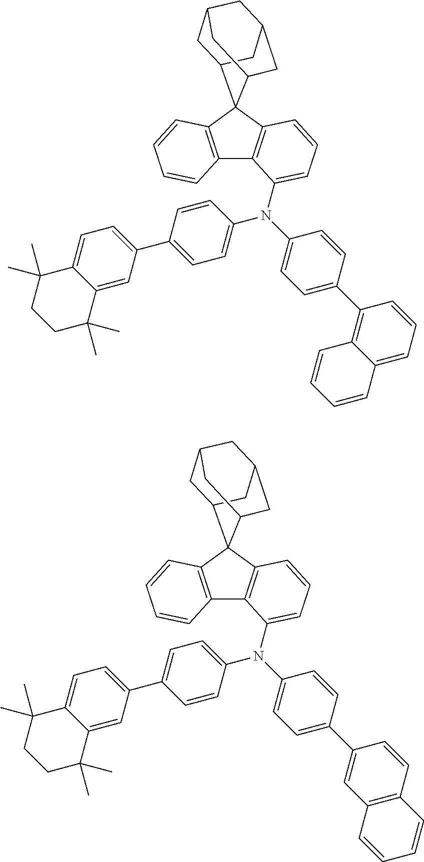 [SMILES: CC1(C)CCC(C)(C)c2cc(-c3ccc(N(c4ccc(-c5cccc6ccccc56)cc4)c4cccc5c4-c4ccccc4C54C5CC6CC(C5)CC4C6)cc3)ccc21 ; CC1(C)CCC(C)(C)c2cc(-c3ccc(N(c4ccc(-c5ccc6ccccc6c5)cc4)c4cccc5c4-c4ccccc4C54C5CC6CC(C5)CC4C6)cc3)ccc21]

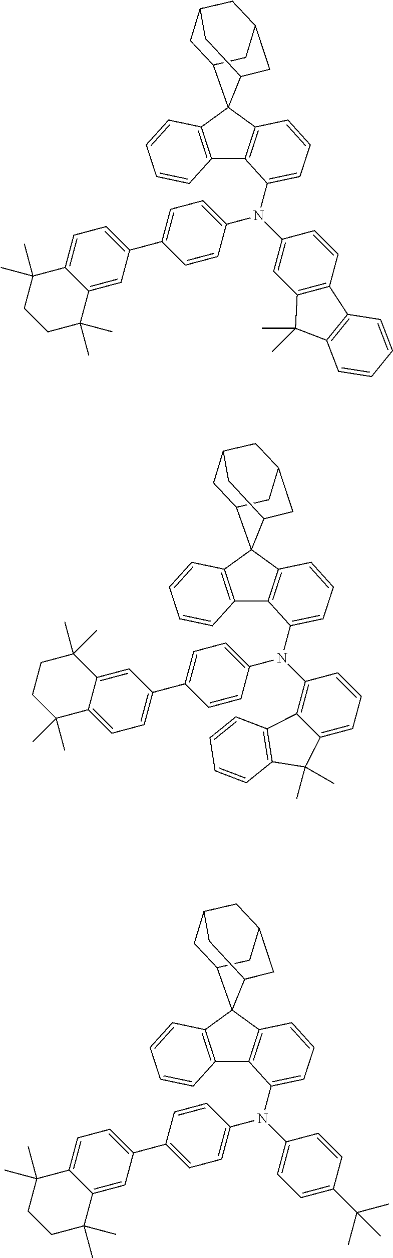 [SMILES: CC1(C)CCC(C)(C)c2cc(-c3ccc(N(c4ccc5c(c4)C(C)(C)c4ccccc4-5)c4cccc5c4-c4ccccc4C54C5CC6CC(C5)CC4C6)cc3)ccc21 ; CC1(C)CCC(C)(C)c2cc(-c3ccc(N(c4cccc5c4-c4ccccc4C5(C)C)c4cccc5c4-c4ccccc4C54C5CC6CC(C5)CC4C6)cc3)ccc21 ; CC(C)(C)c1ccc(N(c2ccc(-c3ccc4c(c3)C(C)(C)CCC4(C)C)cc2)c2cccc3c2-c2ccccc2C32C3CC4CC(C3)CC2C4)cc1]

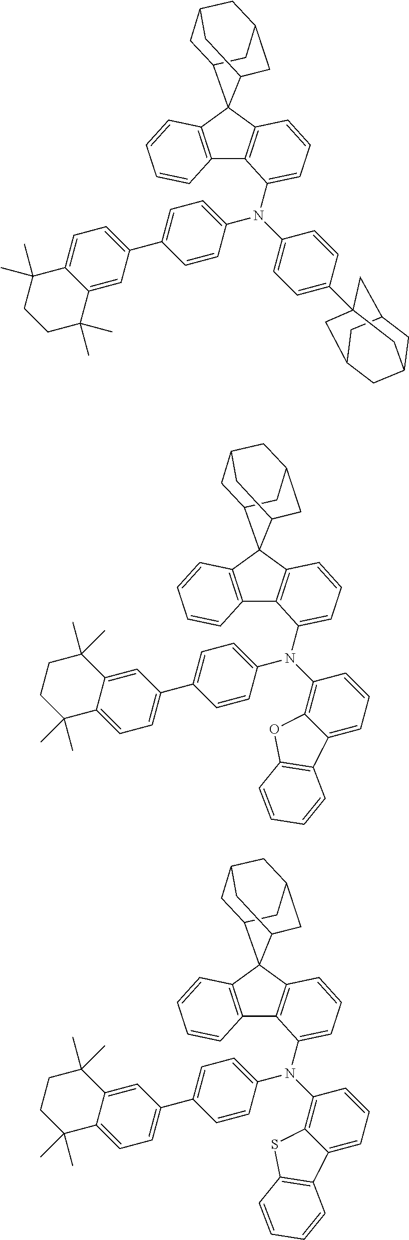

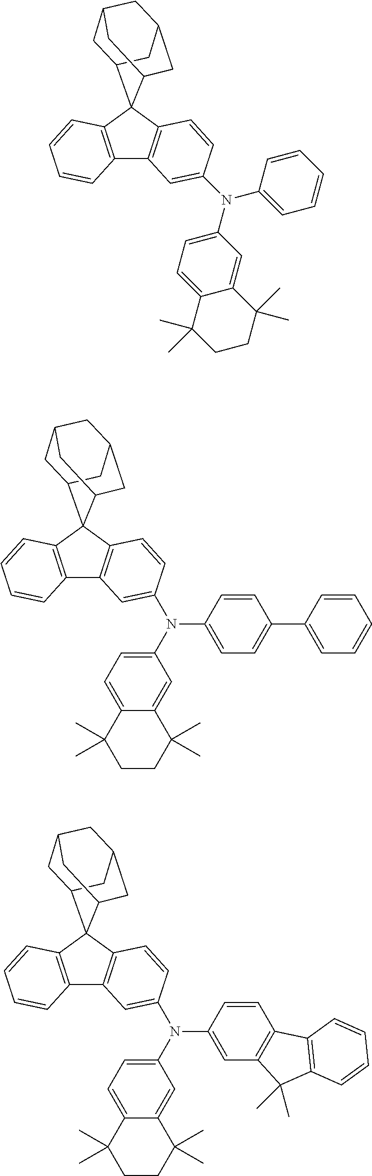 [SMILES: CC1(C)CCC(C)(C)c2cc(N(c3ccccc3)c3ccc4c(c3)-c3ccccc3C43C4CC5CC(C4)CC3C5)ccc21 ; CC1(C)CCC(C)(C)c2cc(N(c3ccc(-c4ccccc4)cc3)c3ccc4c(c3)-c3ccccc3C43C4CC5CC(C4)CC3C5)ccc21 ; CC1(C)CCC(C)(C)c2cc(N(c3ccc4c(c3)-c3ccccc3C43C4CC5CC(C4)CC3C5)c3ccc4c(c3)C(C)(C)c3ccccc3-4)ccc21]

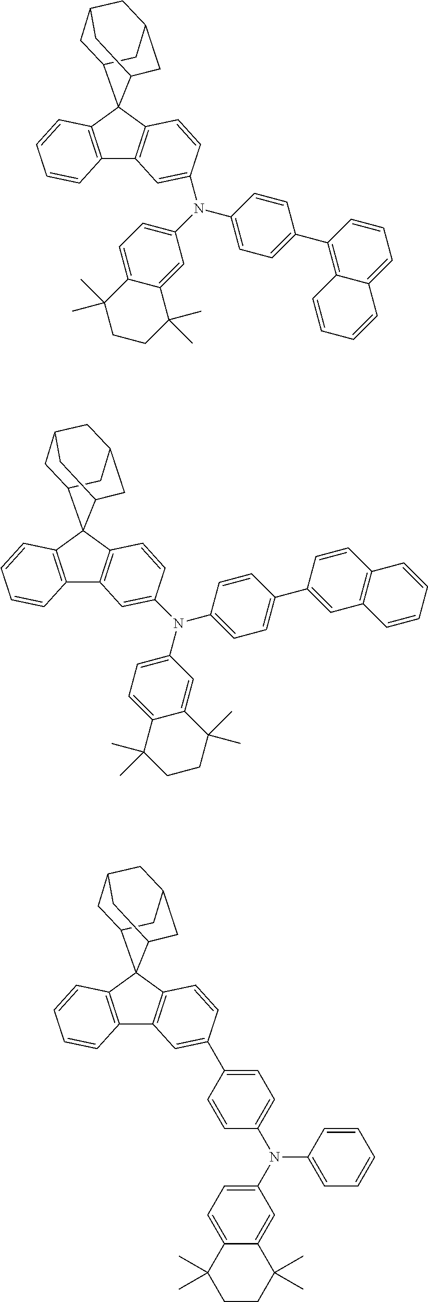 [SMILES: CC1(C)CCC(C)(C)c2cc(N(c3ccc(-c4cccc5ccccc45)cc3)c3ccc4c(c3)-c3ccccc3C43C4CC5CC(C4)CC3C5)ccc21 ; CC1(C)CCC(C)(C)c2cc(N(c3ccc(-c4ccc5ccccc5c4)cc3)c3ccc4c(c3)-c3ccccc3C43C4CC5CC(C4)CC3C5)ccc21 ; CC1(C)CCC(C)(C)c2cc(N(c3ccccc3)c3ccc(-c4ccc5c(c4)-c4ccccc4C54C5CC6CC(C5)CC4C6)cc3)ccc21]

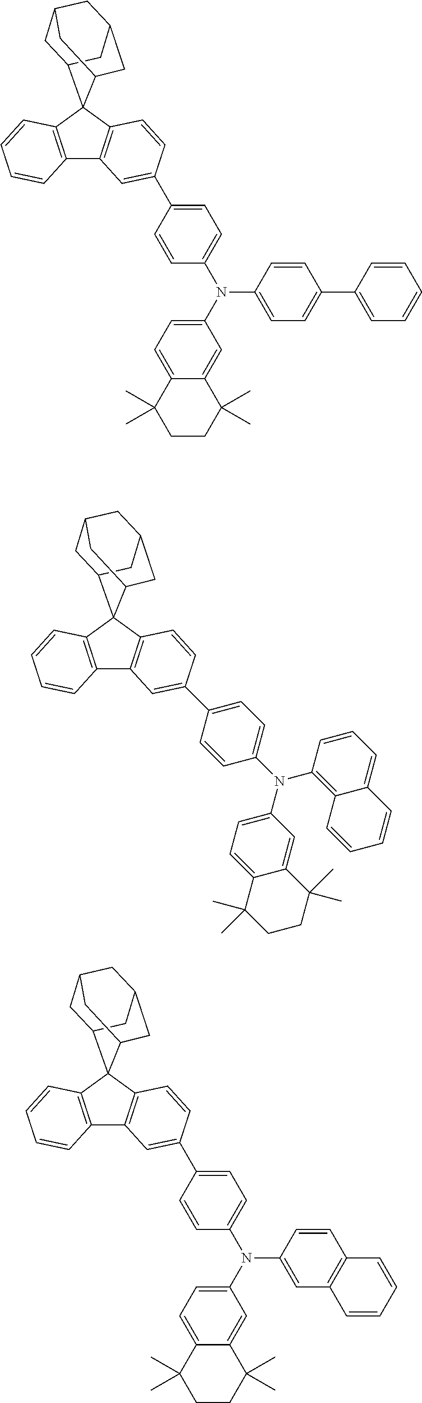 [SMILES: CC1(C)CCC(C)(C)c2cc(N(c3ccc(-c4ccccc4)cc3)c3ccc(-c4ccc5c(c4)-c4ccccc4C54C5CC6CC(C5)CC4C6)cc3)ccc21 ; CC1(C)CCC(C)(C)c2cc(N(c3ccc(-c4ccc5c(c4)-c4ccccc4C54C5CC6CC(C5)CC4C6)cc3)c3cccc4ccccc34)ccc21 ; CC1(C)CCC(C)(C)c2cc(N(c3ccc(-c4ccc5c(c4)-c4ccccc4C54C5CC6CC(C5)CC4C6)cc3)c3ccc4ccccc4c3)ccc21]

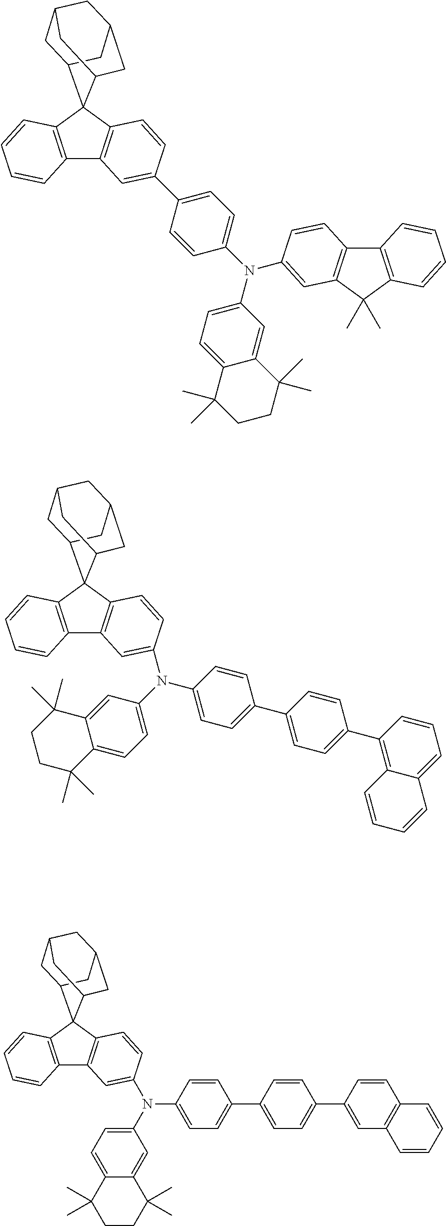 [SMILES: CC1(C)CCC(C)(C)c2cc(N(c3ccc(-c4ccc5c(c4)-c4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc(-c5cccc6ccccc56)cc4)cc3)c3ccc4c(c3)-c3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3ccc4c(c3)-c3ccccc3C43C4CC5CC(C4)CC3C5)ccc21]

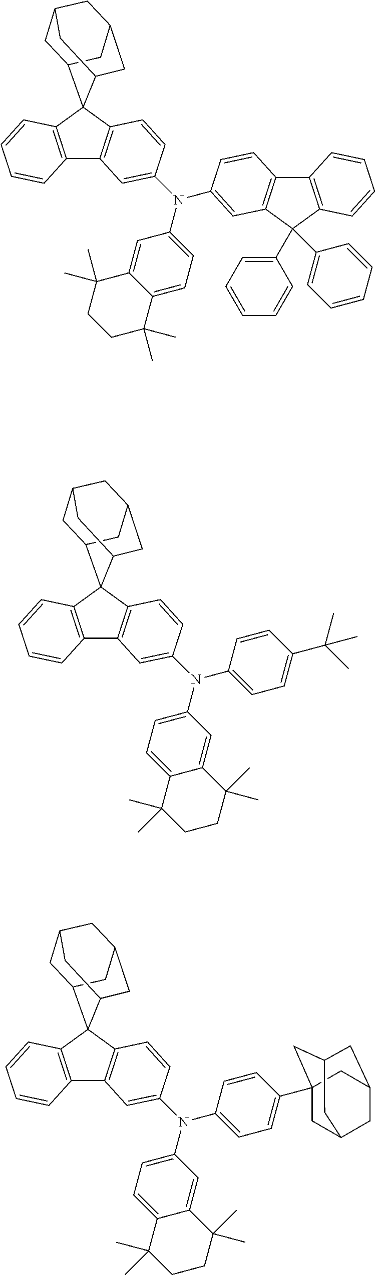 [SMILES: CC1(C)CCC(C)(C)c2cc(N(c3ccc4c(c3)-c3ccccc3C43C4CC5CC(C4)CC3C5)c3ccc4c(c3)C(c3ccccc3)(c3ccccc3)c3ccccc3-4)ccc21 ; CC(C)(C)c1ccc(N(c2ccc3c(c2)-c2ccccc2C32C3CC4CC(C3)CC2C4)c2ccc3c(c2)C(C)(C)CCC3(C)C)cc1 ; CC1(C)CCC(C)(C)c2cc(N(c3ccc(C45CC6CC(CC(C6)C4)C5)cc3)c3ccc4c(c3)-c3ccccc3C43C4CC5CC(C4)CC3C5)ccc21]

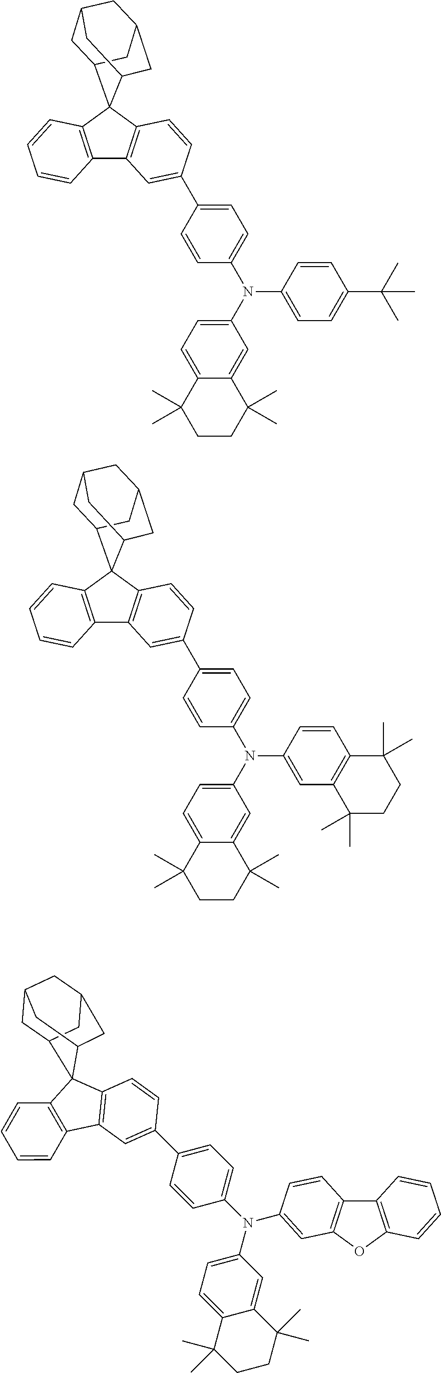 [SMILES: CC(C)(C)c1ccc(N(c2ccc(-c3ccc4c(c3)-c3ccccc3C43C4CC5CC(C4)CC3C5)cc2)c2ccc3c(c2)C(C)(C)CCC3(C)C)cc1 ; CC1(C)CCC(C)(C)c2cc(N(c3ccc(-c4ccc5c(c4)-c4ccccc4C54C5CC6CC(C5)CC4C6)cc3)c3ccc4c(c3)C(C)(C)CCC4(C)C)ccc21 ; CC1(C)CCC(C)(C)c2cc(N(c3ccc(-c4ccc5c(c4)-c4ccccc4C54C5CC6CC(C5)CC4C6)cc3)c3ccc4c(c3)oc3ccccc34)ccc21]

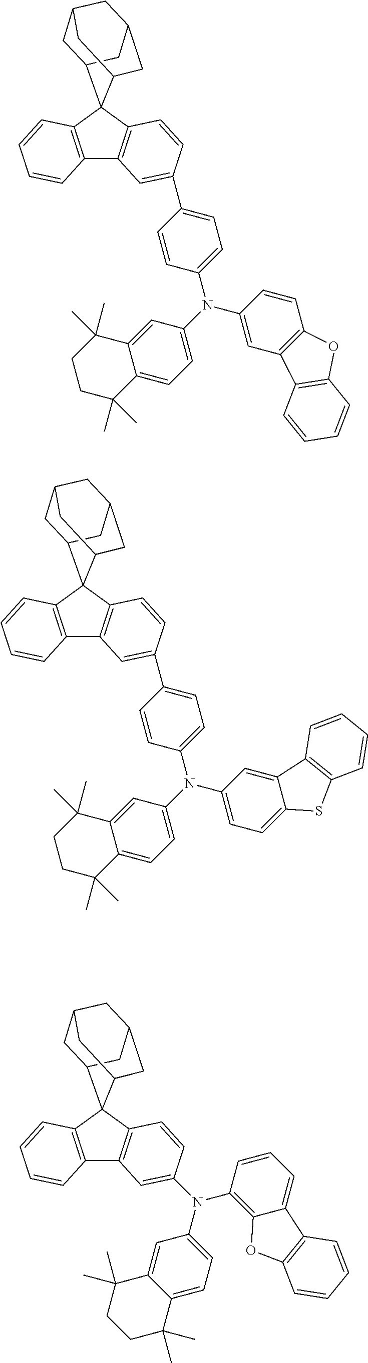 [SMILES: CC1(C)CCC(C)(C)c2cc(N(c3ccc(-c4ccc5c(c4)-c4ccccc4C54C5CC6CC(C5)CC4C6)cc3)c3ccc4oc5ccccc5c4c3)ccc21 ; CC1(C)CCC(C)(C)c2cc(N(c3ccc(-c4ccc5c(c4)-c4ccccc4C54C5CC6CC(C5)CC4C6)cc3)c3ccc4sc5ccccc5c4c3)ccc21 ; CC1(C)CCC(C)(C)c2cc(N(c3ccc4c(c3)-c3ccccc3C43C4CC5CC(C4)CC3C5)c3cccc4c3oc3ccccc34)ccc21]

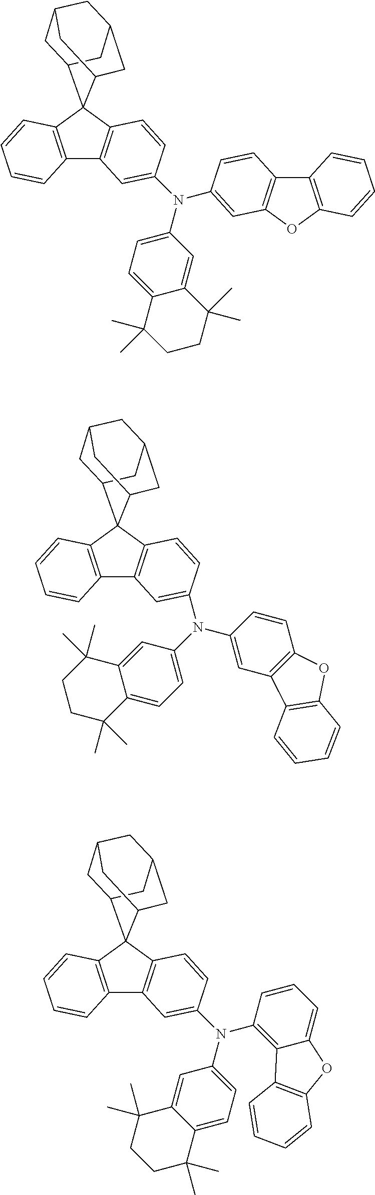 [SMILES: CC1(C)CCC(C)(C)c2cc(N(c3ccc4c(c3)-c3ccccc3C43C4CC5CC(C4)CC3C5)c3ccc4c(c3)oc3ccccc34)ccc21 ; CC1(C)CCC(C)(C)c2cc(N(c3ccc4c(c3)-c3ccccc3C43C4CC5CC(C4)CC3C5)c3ccc4oc5ccccc5c4c3)ccc21 ; CC1(C)CCC(C)(C)c2cc(N(c3ccc4c(c3)-c3ccccc3C43C4CC5CC(C4)CC3C5)c3cccc4oc5ccccc5c34)ccc21]

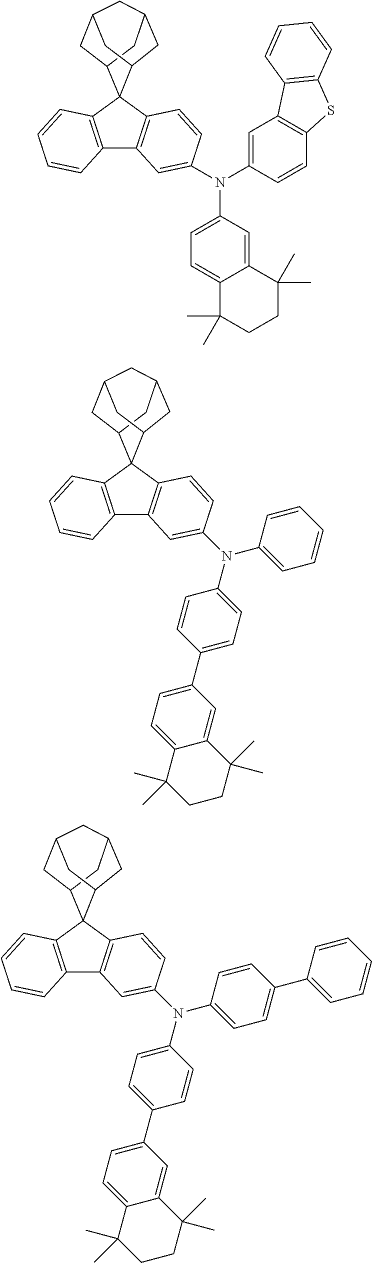 [SMILES: CC1(C)CCC(C)(C)c2cc(N(c3ccc4c(c3)-c3ccccc3C43C4CC5CC(C4)CC3C5)c3ccc4sc5ccccc5c4c3)ccc21 ; CC1(C)CCC(C)(C)c2cc(-c3ccc(N(c4ccccc4)c4ccc5c(c4)-c4ccccc4C54C5CC6CC(C5)CC4C6)cc3)ccc21 ; CC1(C)CCC(C)(C)c2cc(-c3ccc(N(c4ccc(-c5ccccc5)cc4)c4ccc5c(c4)-c4ccccc4C54C5CC6CC(C5)CC4C6)cc3)ccc21]

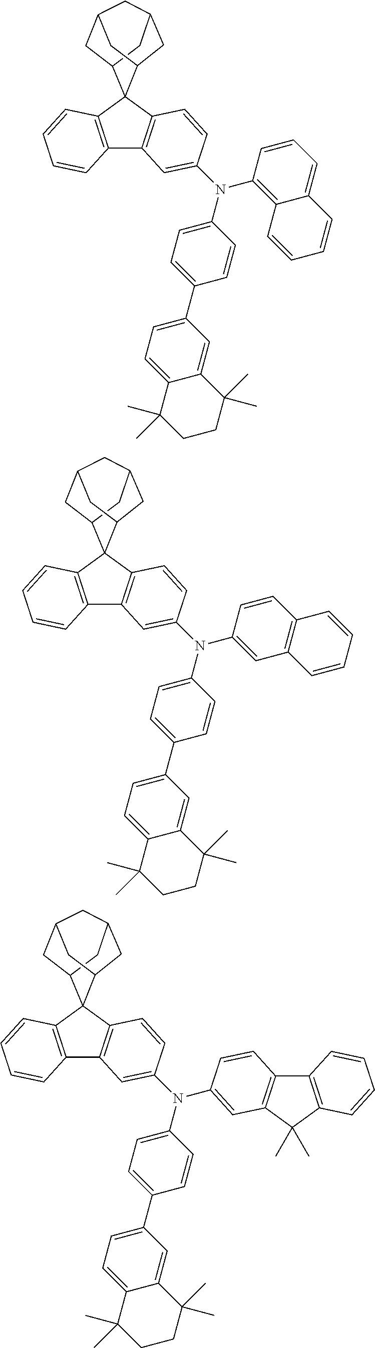

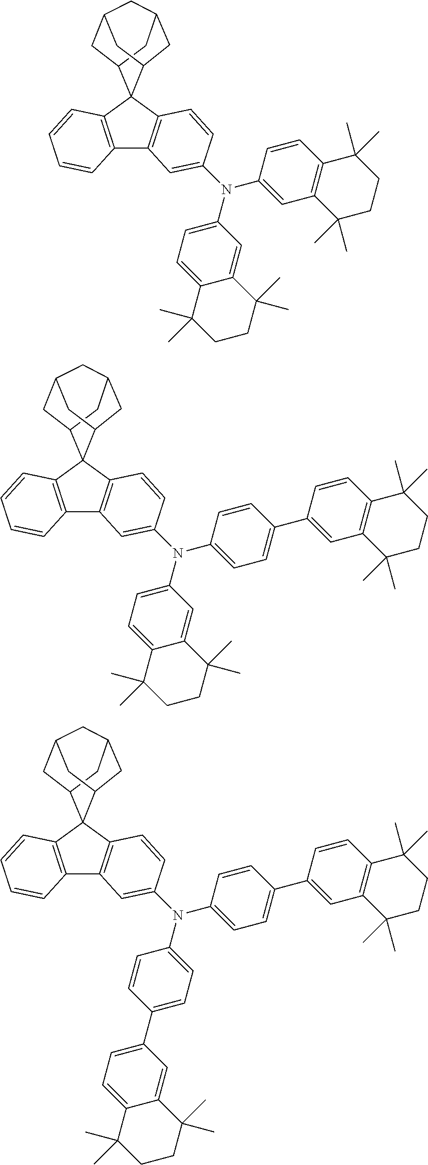

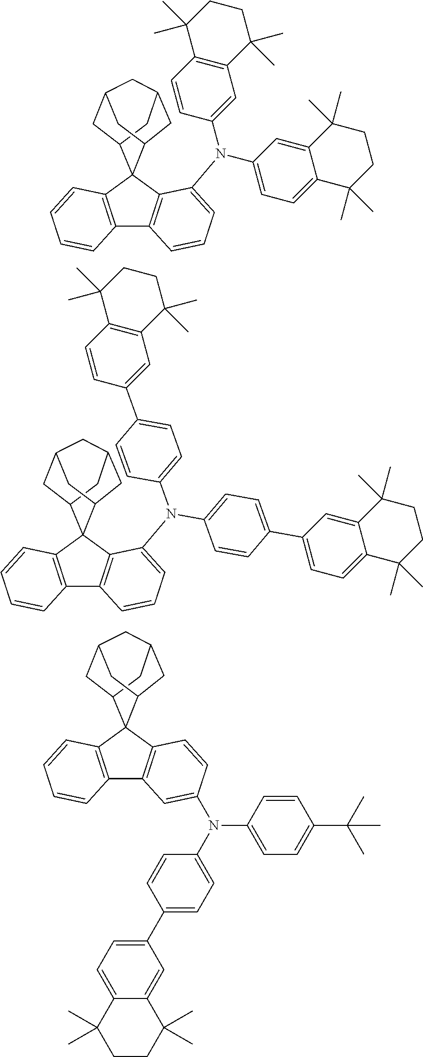

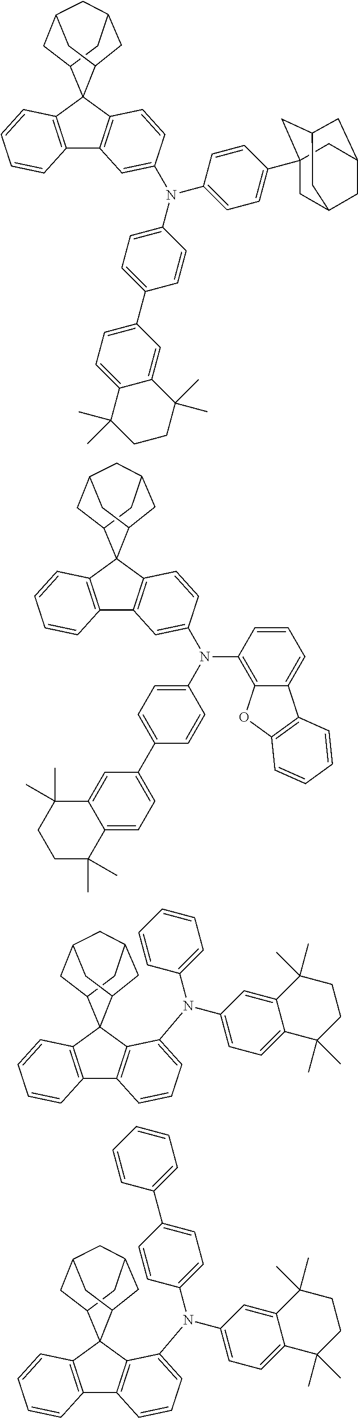

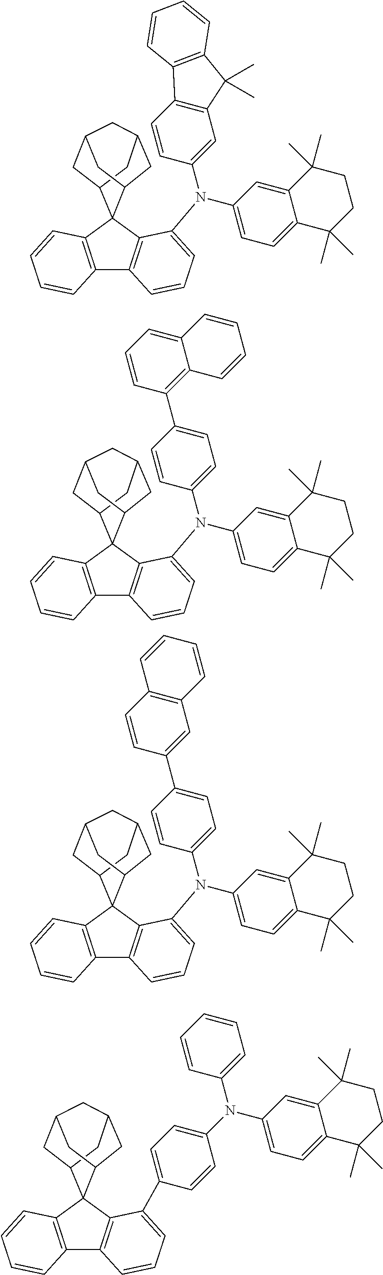

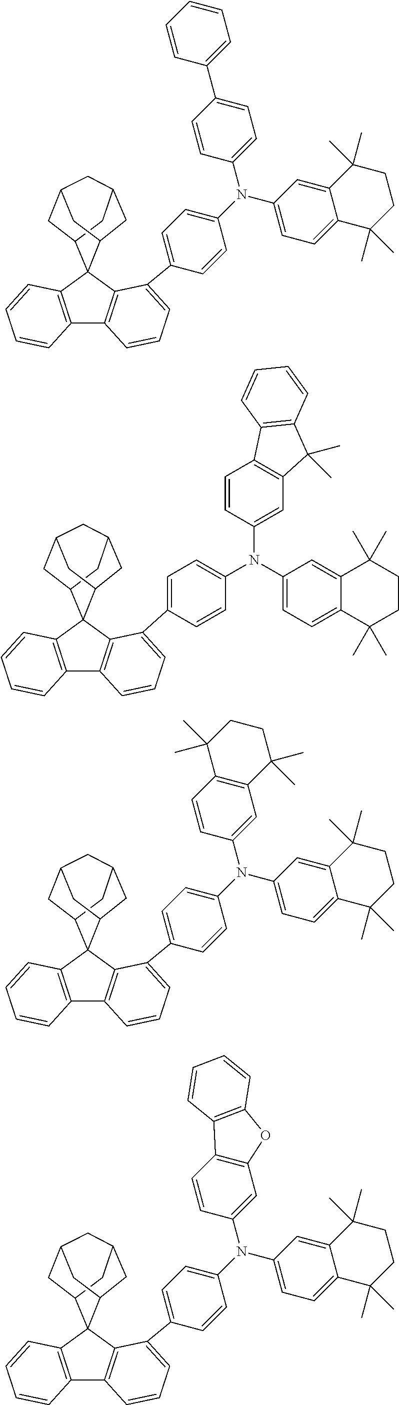

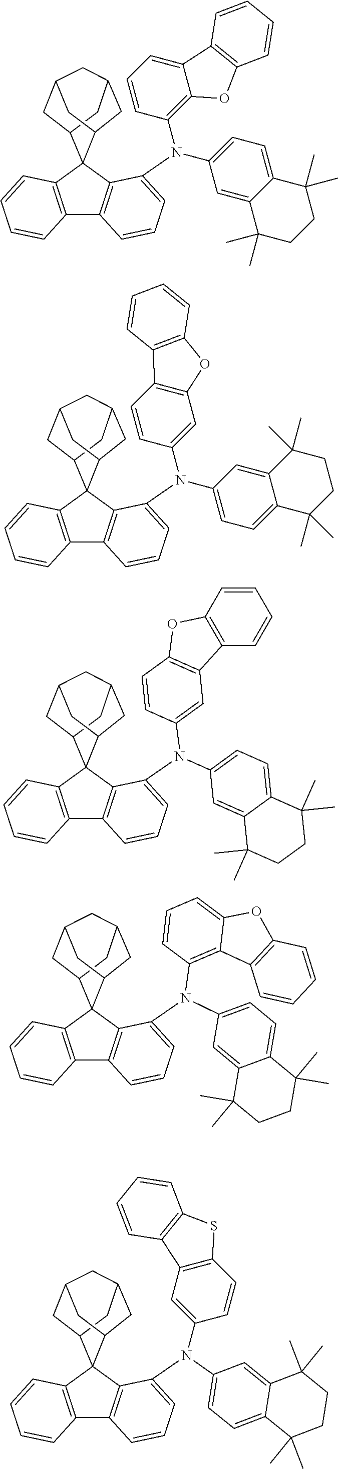

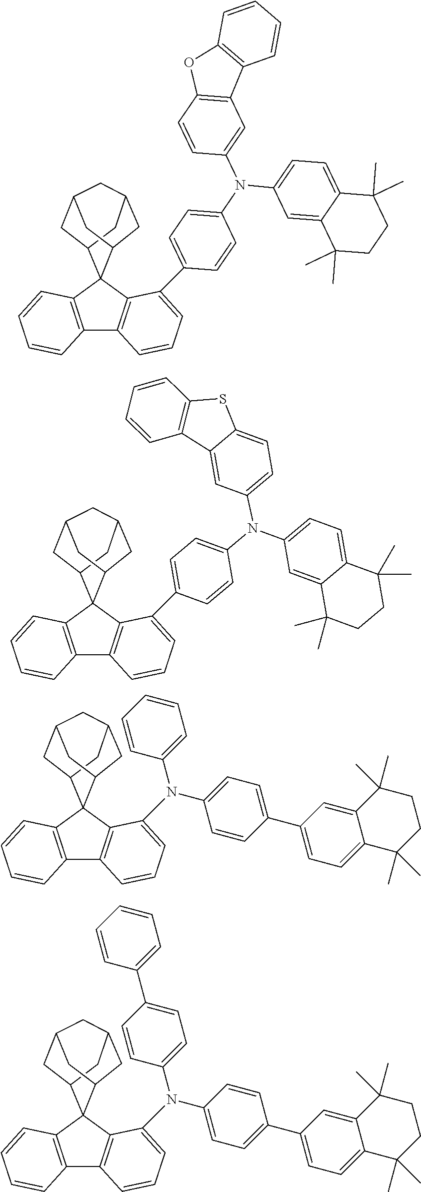

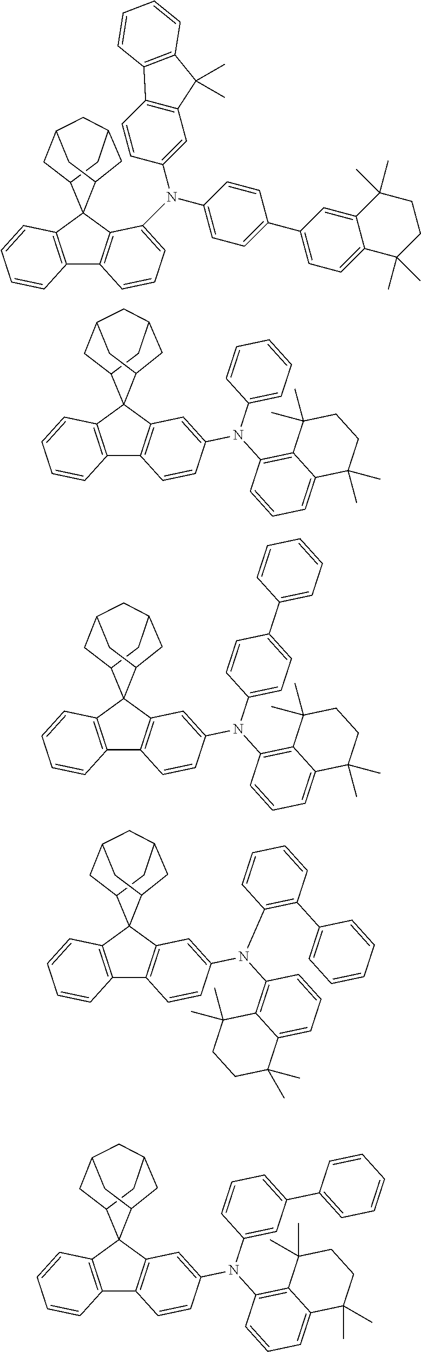

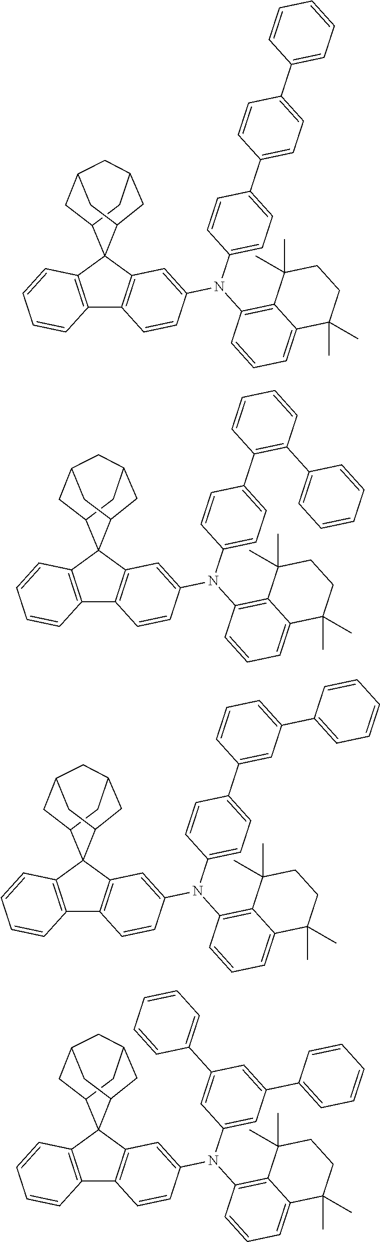

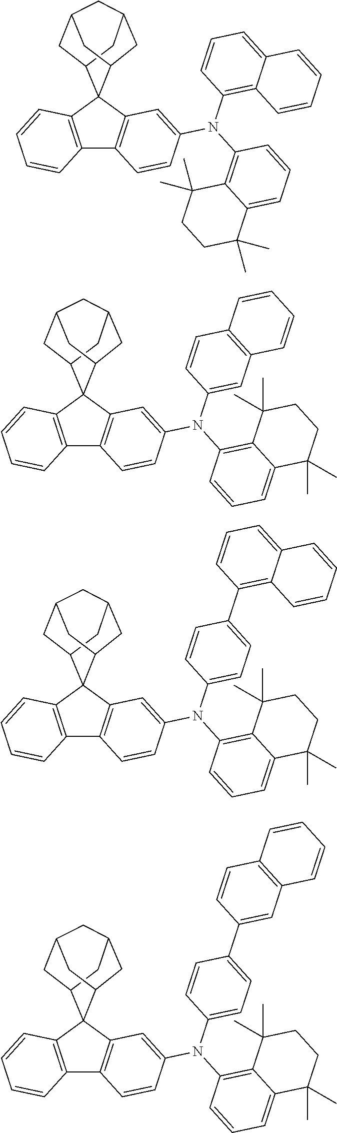

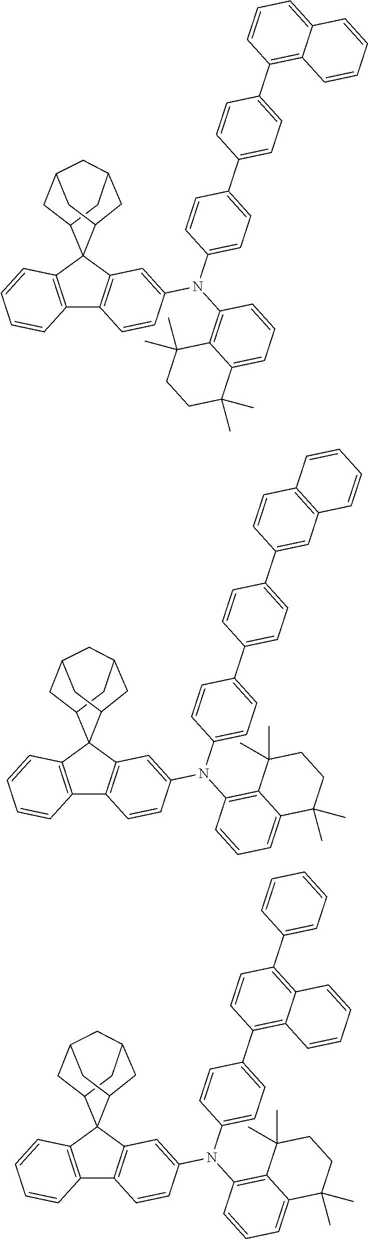

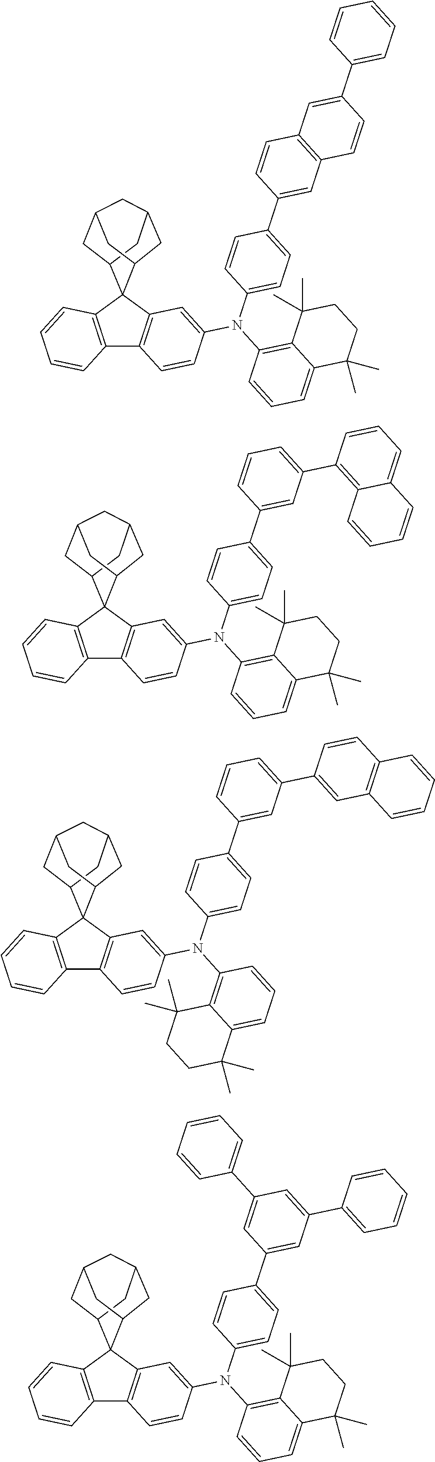

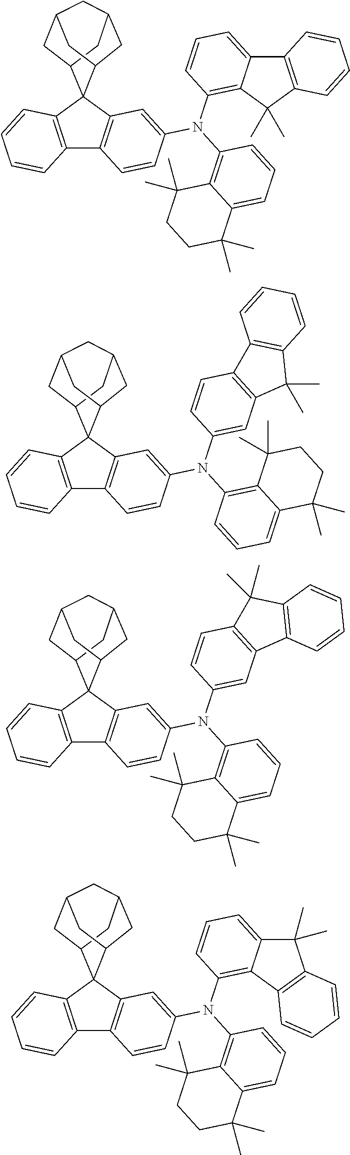

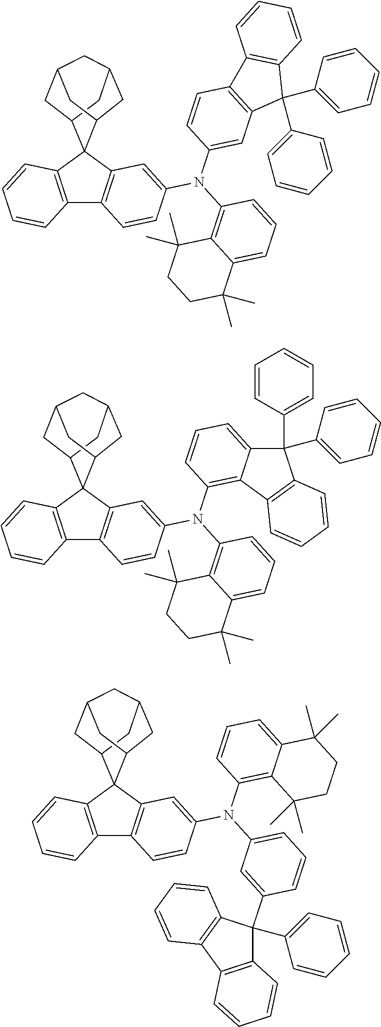

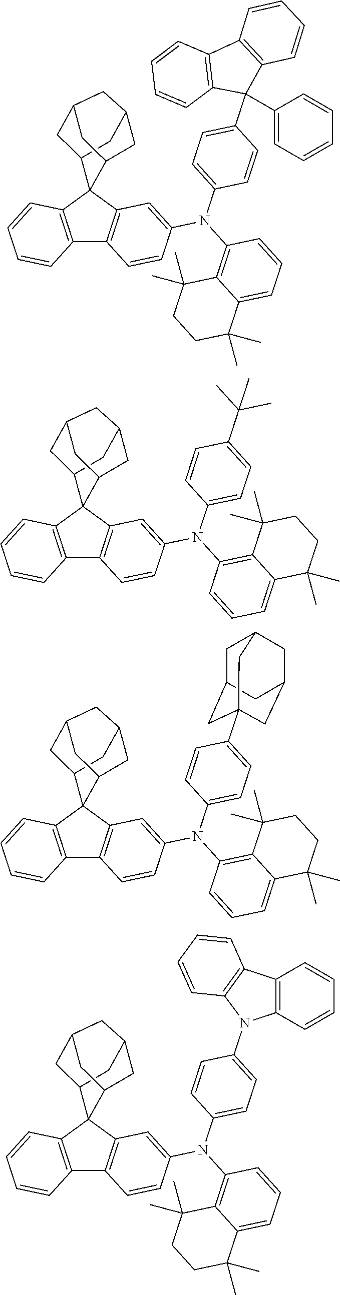

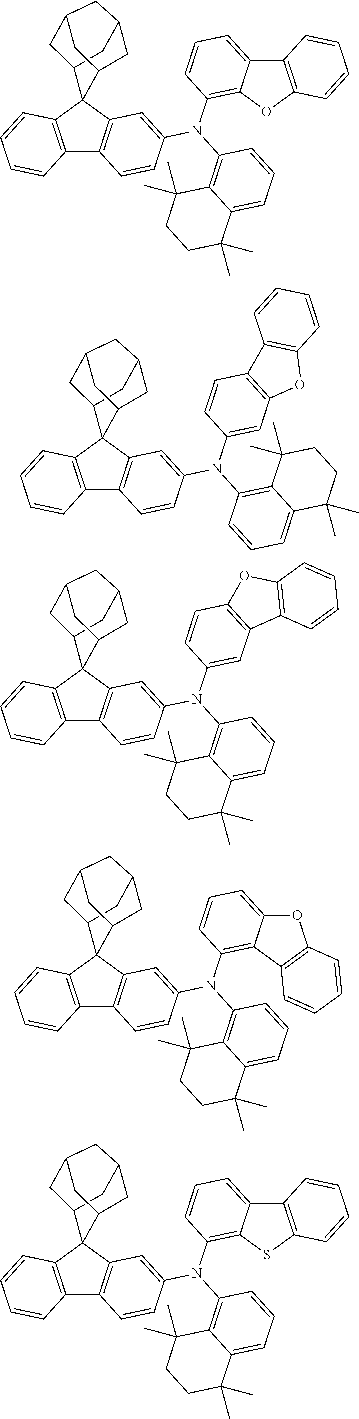

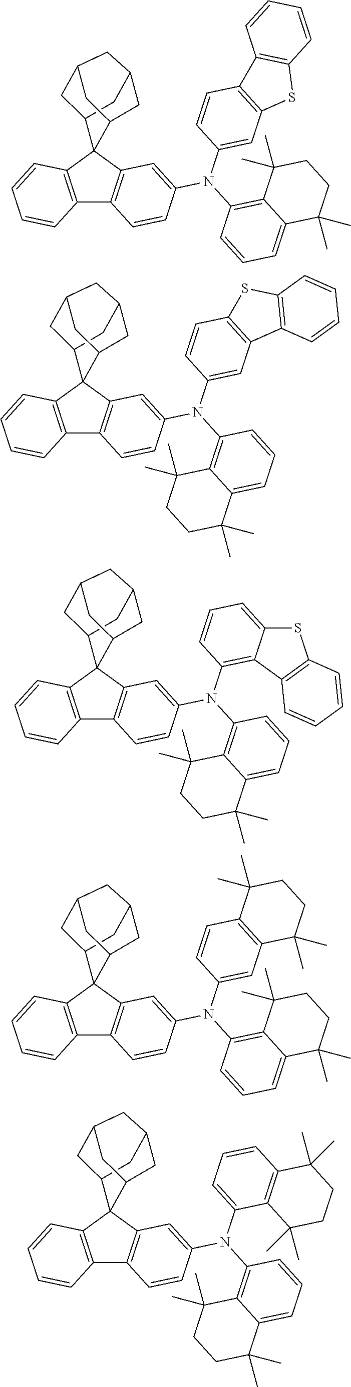

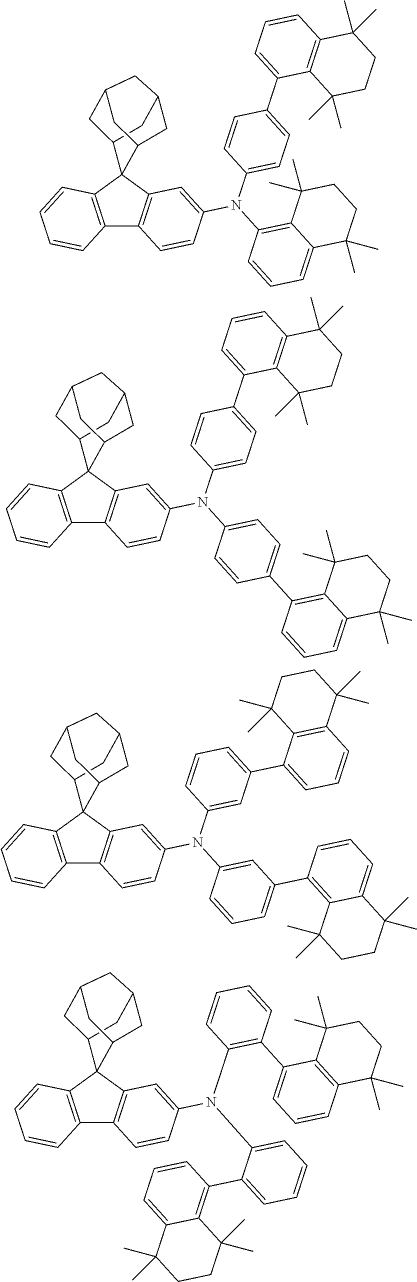

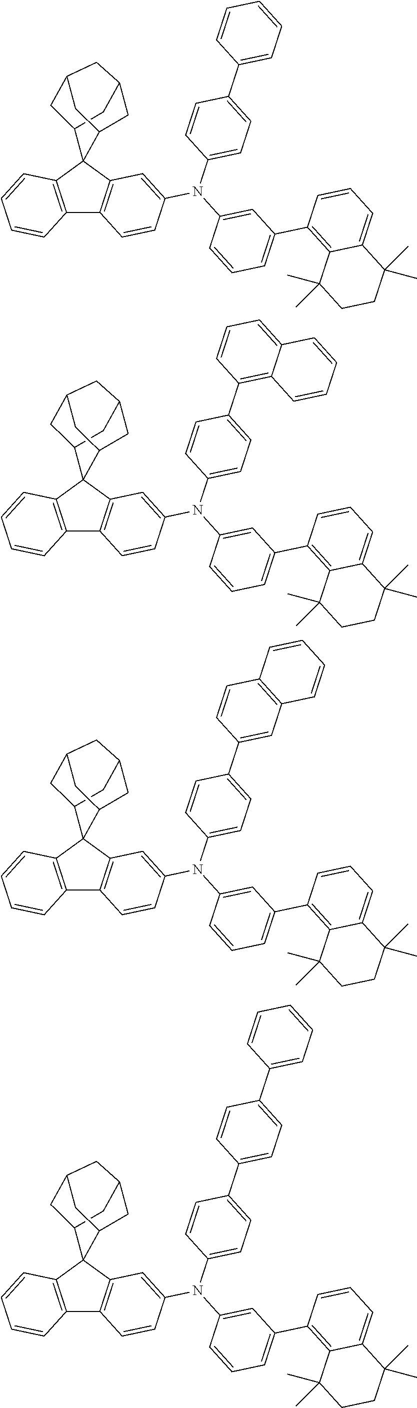

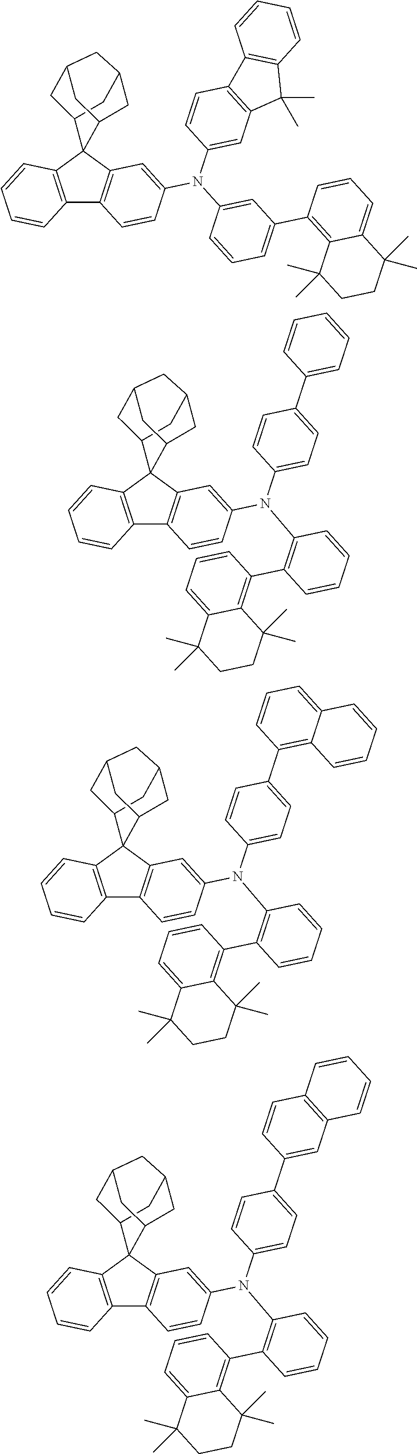

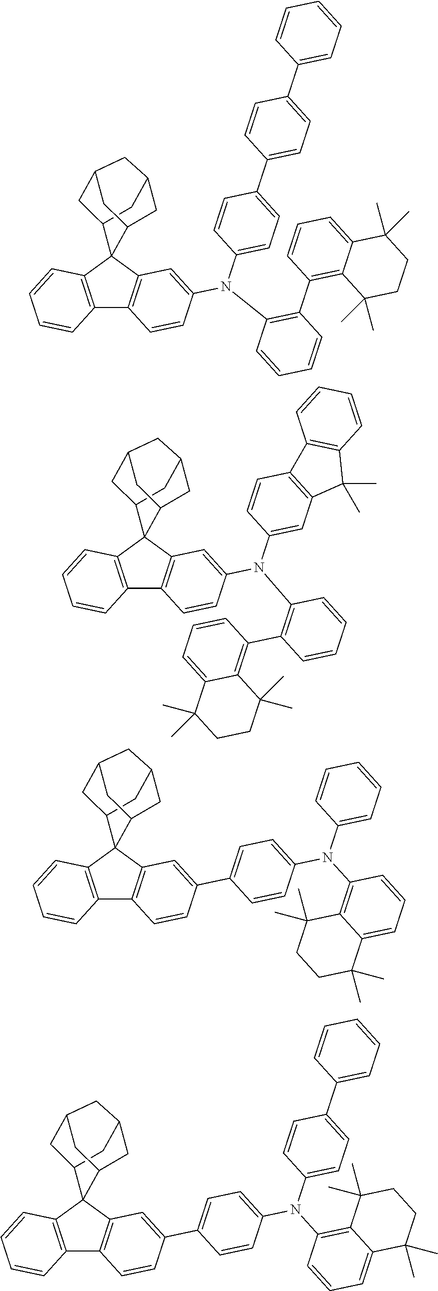

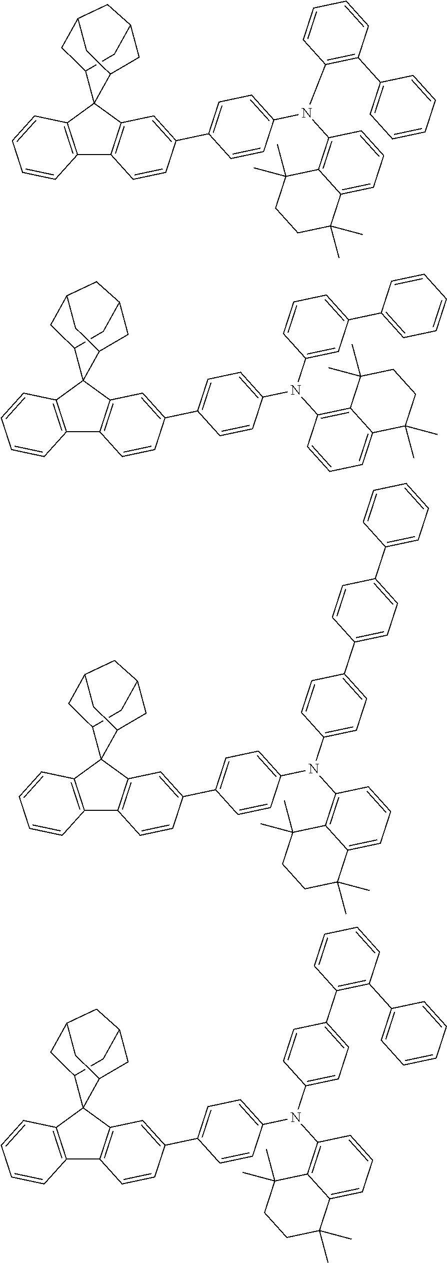

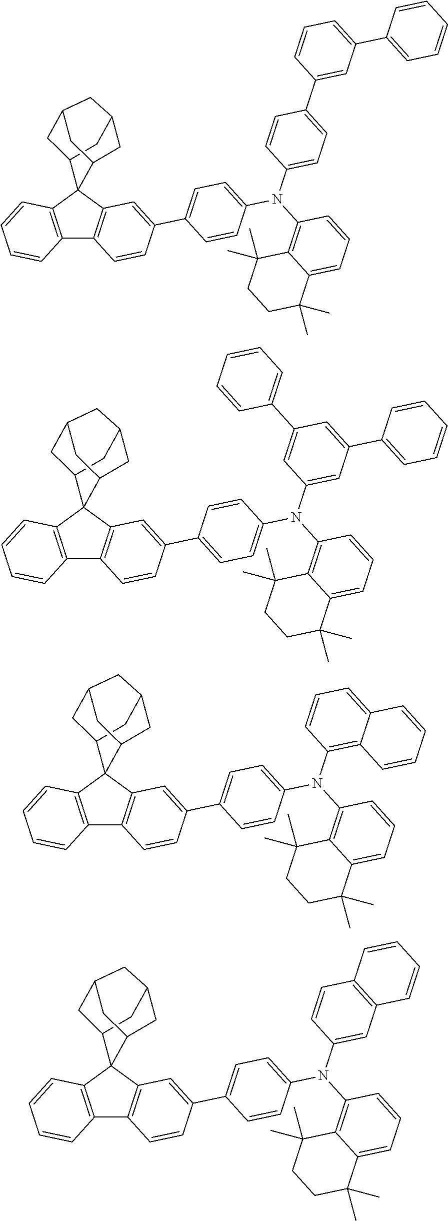

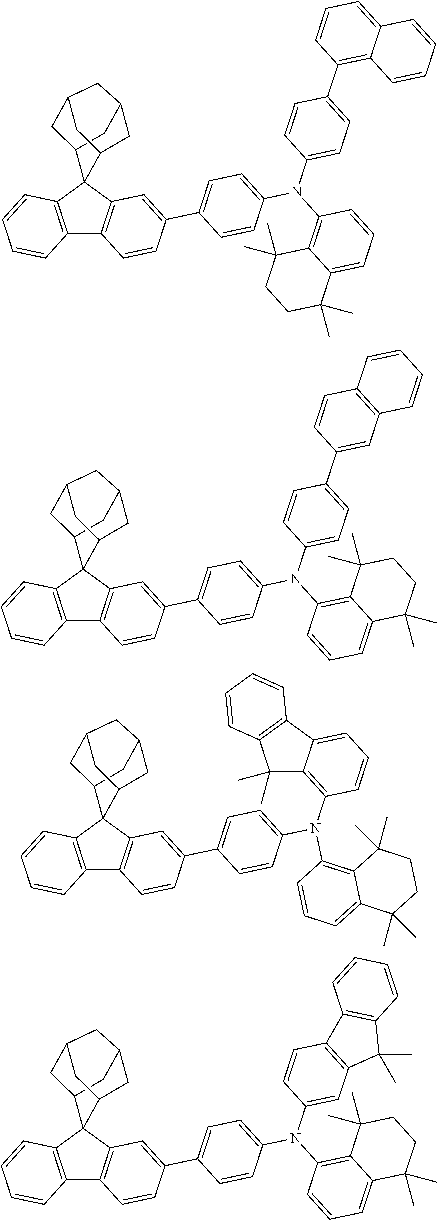

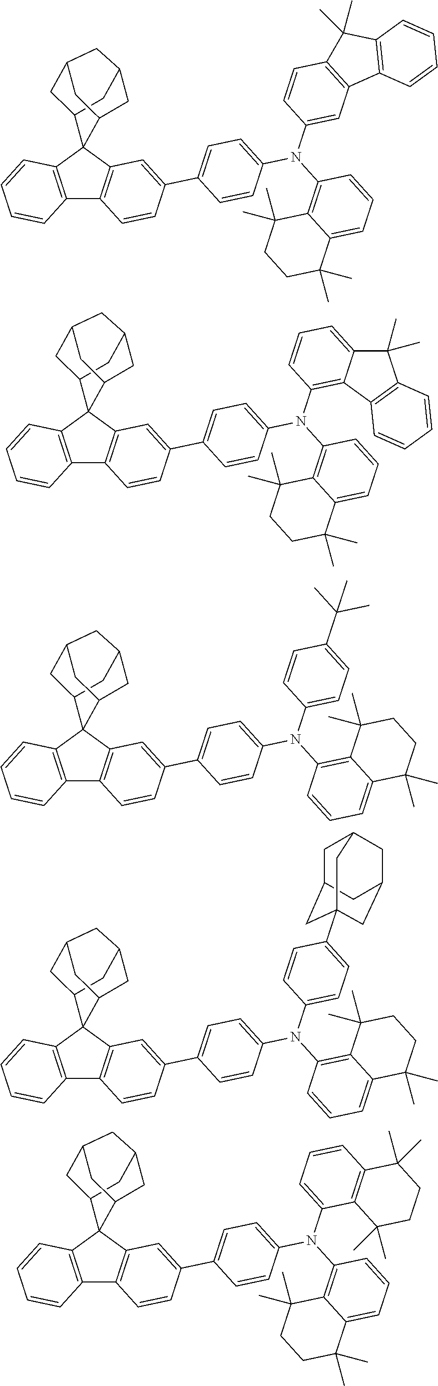

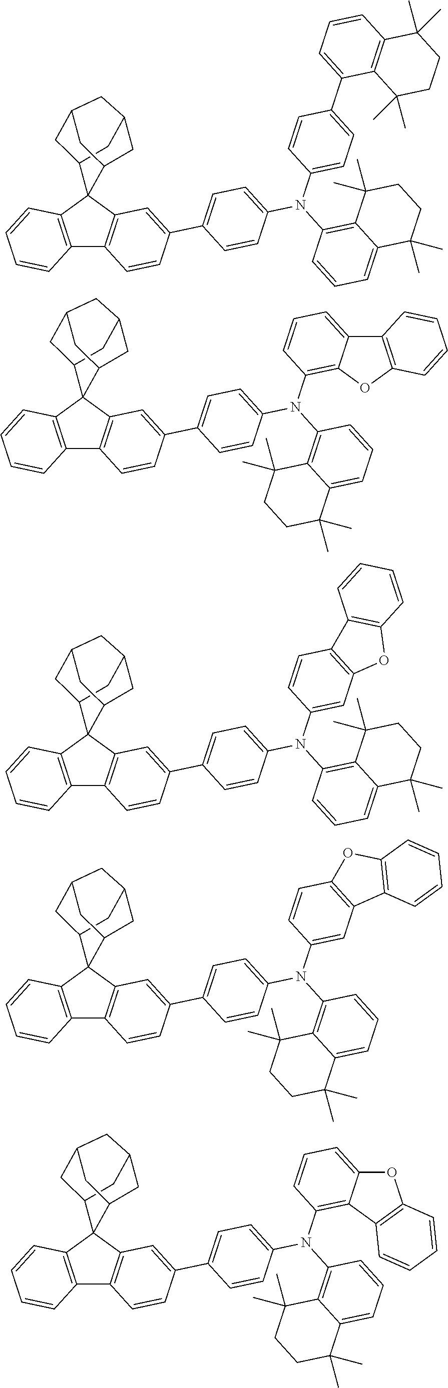

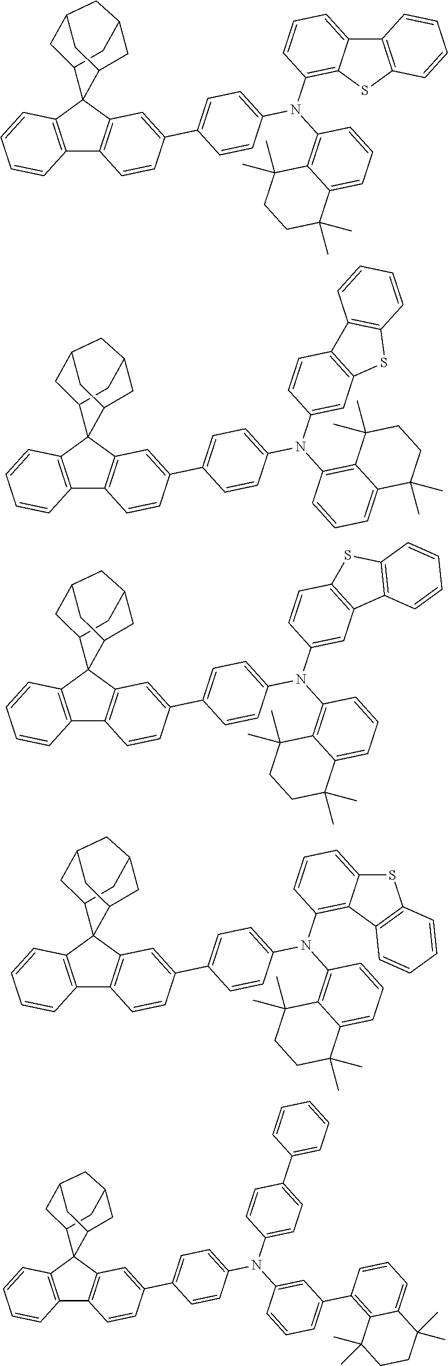

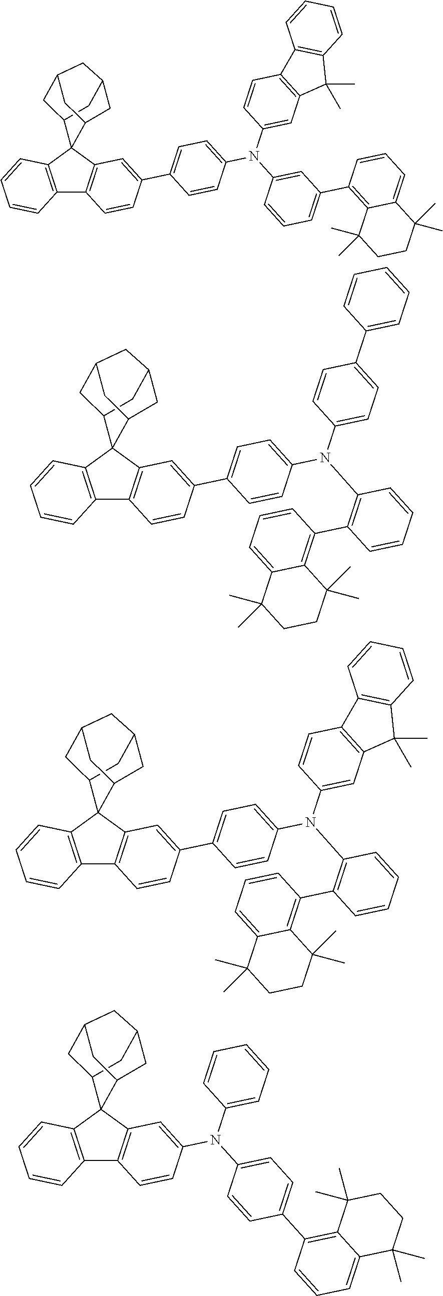

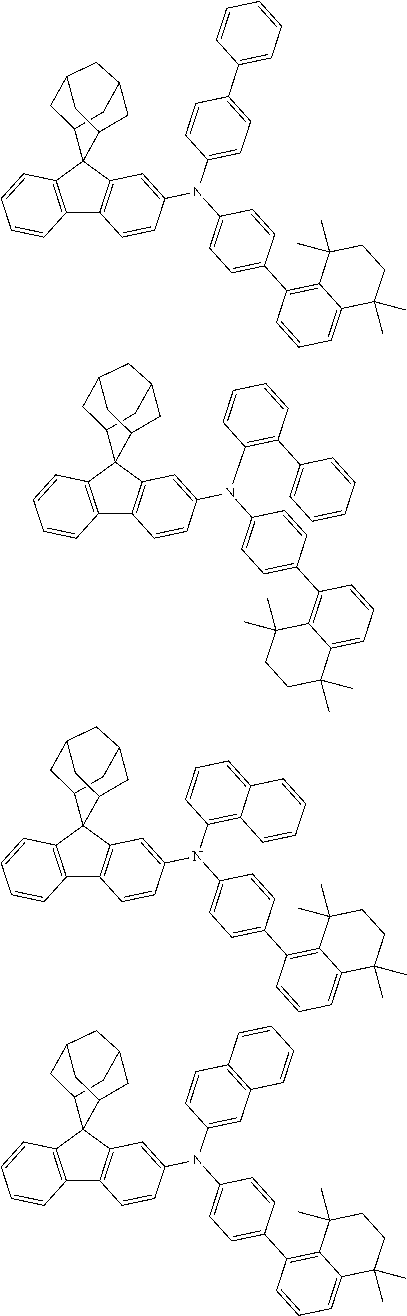

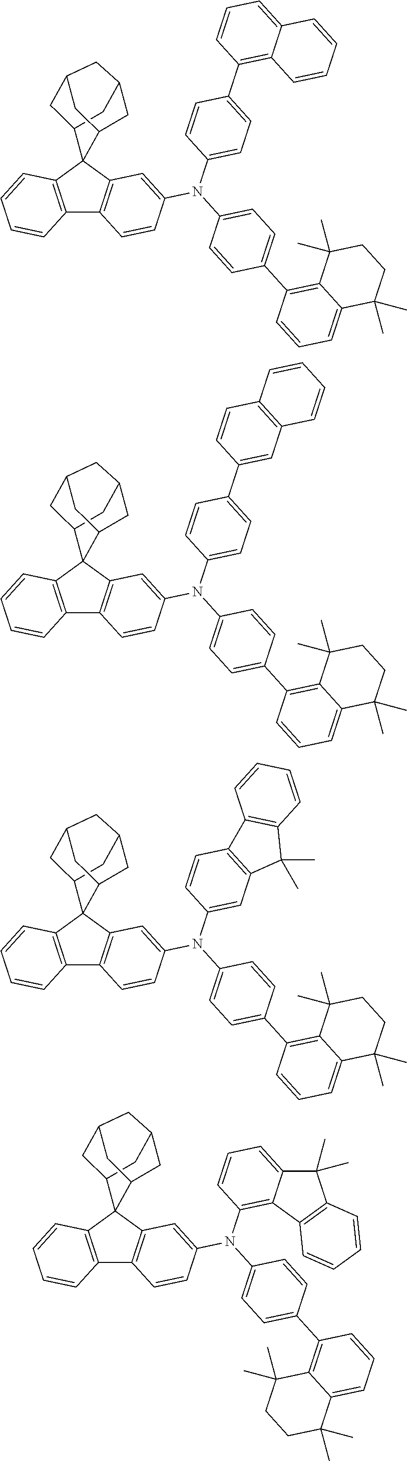

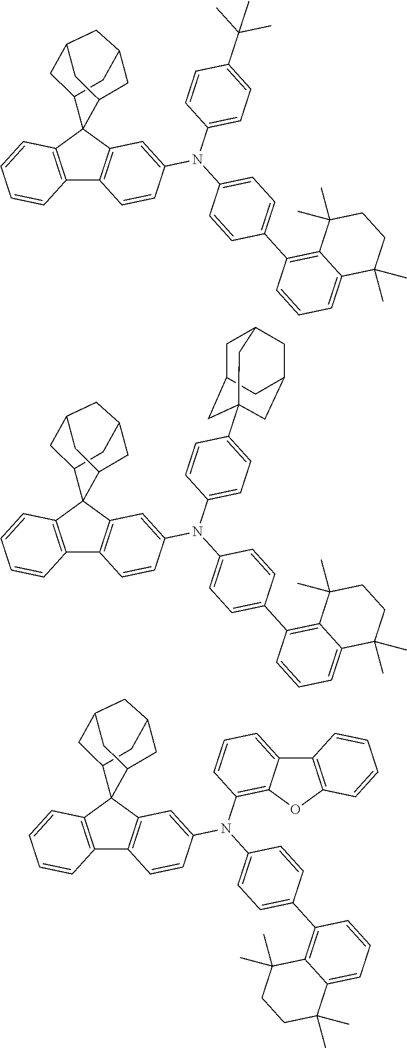

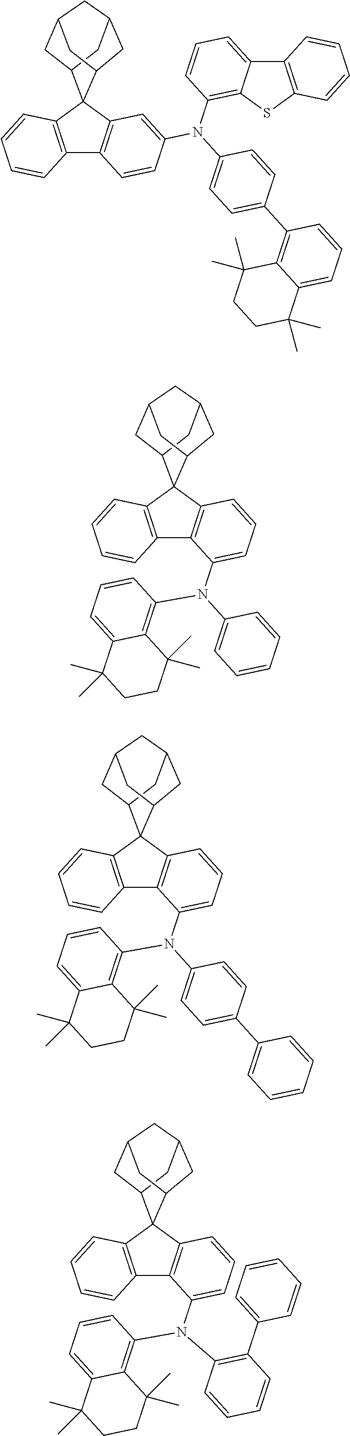

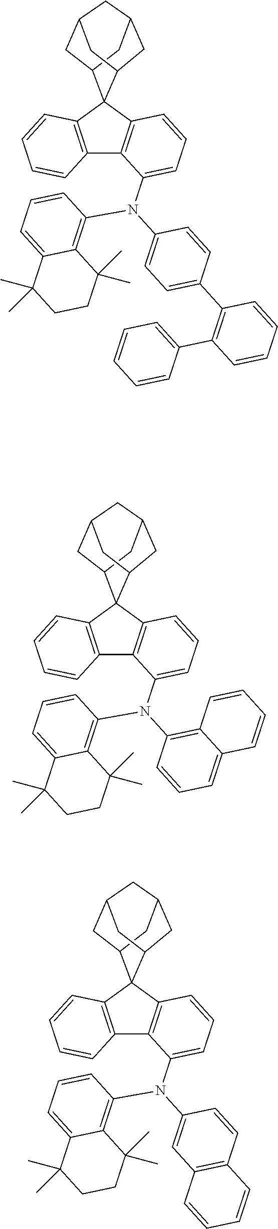 [SMILES: CC1(C)CCC(C)(C)c2c(N(c3ccc(-c4ccccc4-c4ccccc4)cc3)c3cccc4c3-c3ccccc3C43C4CC5CC(C4)CC3C5)cccc21 ; CC1(C)CCC(C)(C)c2c(N(c3cccc4c3-c3ccccc3C43C4CC5CC(C4)CC3C5)c3cccc4ccccc34)cccc21 ; CC1(C)CCC(C)(C)c2c(N(c3ccc4ccccc4c3)c3cccc4c3-c3ccccc3C43C4CC5CC(C4)CC3C5)cccc21]

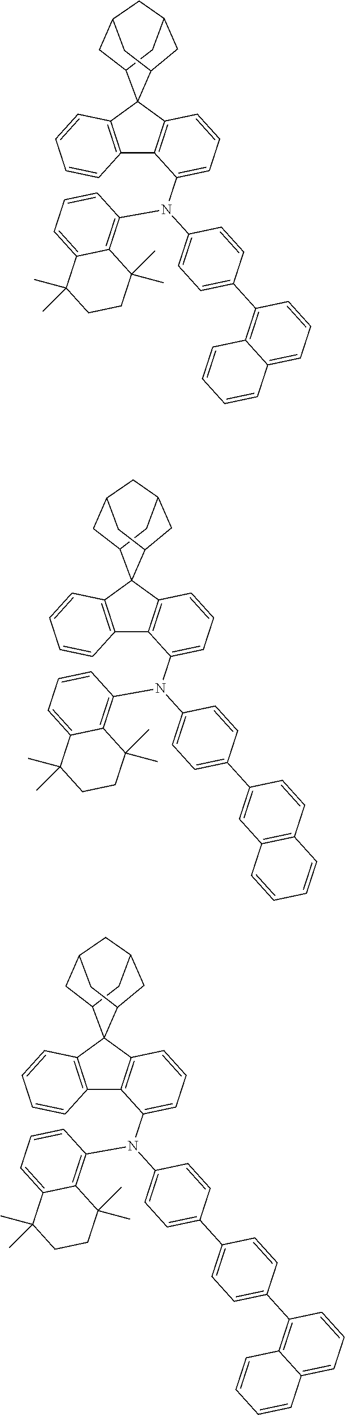 [SMILES: CC1(C)CCC(C)(C)c2c(N(c3ccc(-c4cccc5ccccc45)cc3)c3cccc4c3-c3ccccc3C43C4CC5CC(C4)CC3C5)cccc21 ; CC1(C)CCC(C)(C)c2c(N(c3ccc(-c4ccc5ccccc5c4)cc3)c3cccc4c3-c3ccccc3C43C4CC5CC(C4)CC3C5)cccc21 ; CC1(C)CCC(C)(C)c2c(N(c3ccc(-c4ccc(-c5cccc6ccccc56)cc4)cc3)c3cccc4c3-c3ccccc3C43C4CC5CC(C4)CC3C5)cccc21]

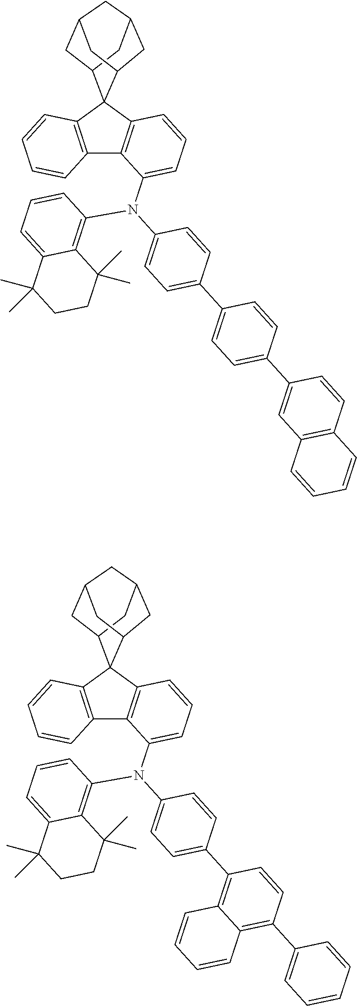 [SMILES: CC1(C)CCC(C)(C)c2c(N(c3ccc(-c4ccc(-c5ccc6ccccc6c5)cc4)cc3)c3cccc4c3-c3ccccc3C43C4CC5CC(C4)CC3C5)cccc21 ; CC1(C)CCC(C)(C)c2c(N(c3ccc(-c4ccc(-c5ccccc5)c5ccccc45)cc3)c3cccc4c3-c3ccccc3C43C4CC5CC(C4)CC3C5)cccc21]

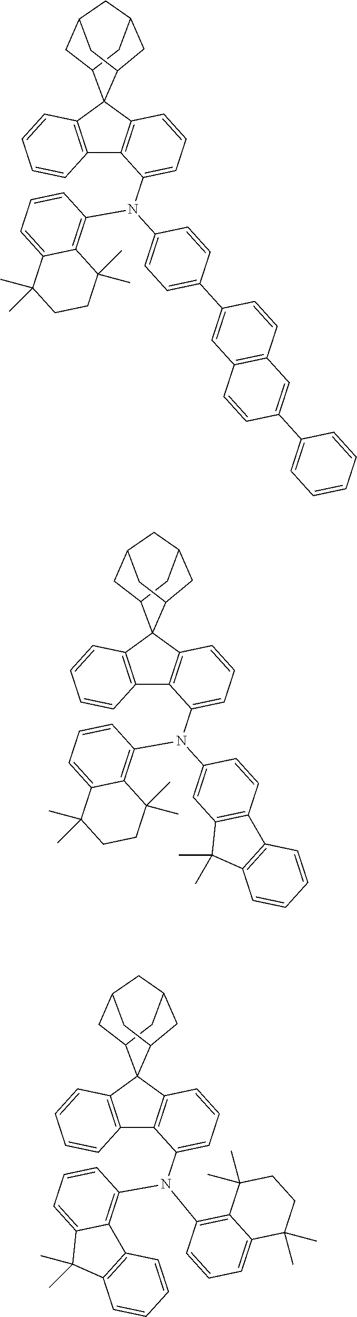

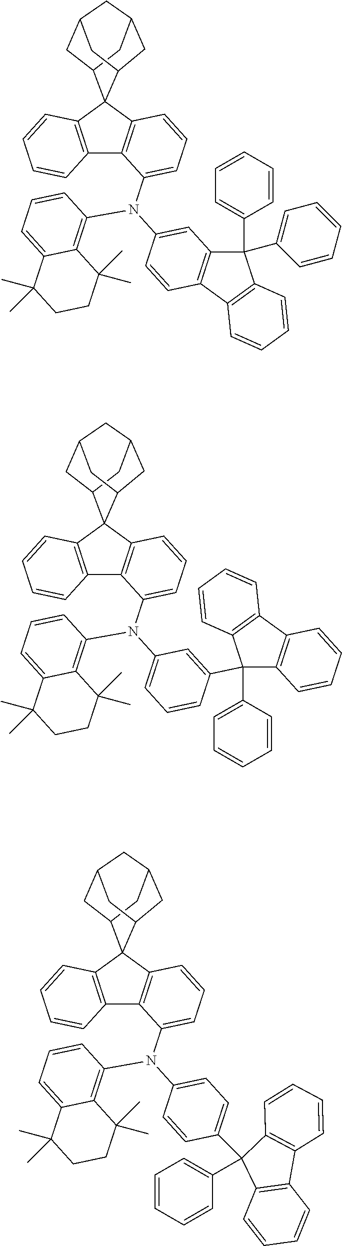 [SMILES: CC1(C)CCC(C)(C)c2c(N(c3ccc4c(c3)C(c3ccccc3)(c3ccccc3)c3ccccc3-4)c3cccc4c3-c3ccccc3C43C4CC5CC(C4)CC3C5)cccc21 ; CC1(C)CCC(C)(C)c2c(N(c3cccc(C4(c5ccccc5)c5ccccc5-c5ccccc54)c3)c3cccc4c3-c3ccccc3C43C4CC5CC(C4)CC3C5)cccc21 ; CC1(C)CCC(C)(C)c2c(N(c3ccc(C4(c5ccccc5)c5ccccc5-c5ccccc54)cc3)c3cccc4c3-c3ccccc3C43C4CC5CC(C4)CC3C5)cccc21]

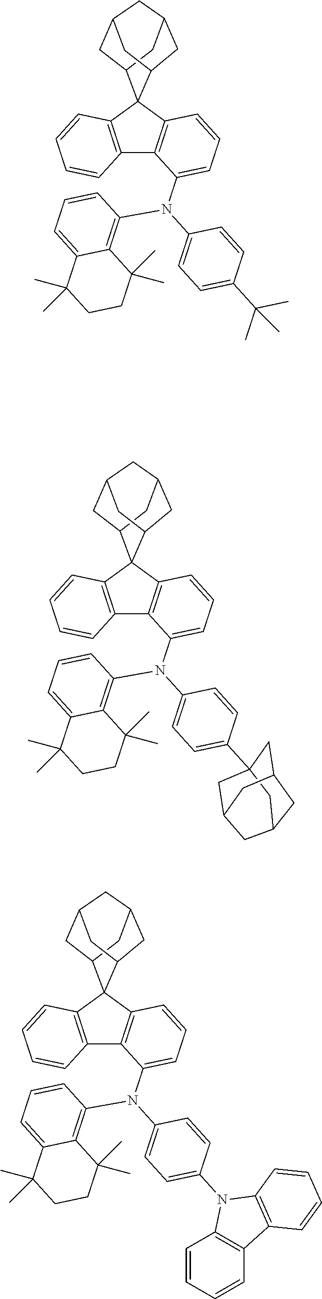

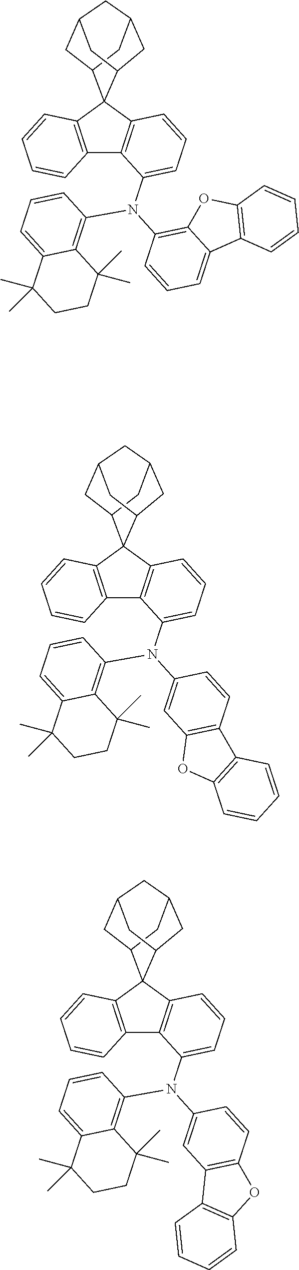 [SMILES: CC1(C)CCC(C)(C)c2c(N(c3cccc4c3-c3ccccc3C43C4CC5CC(C4)CC3C5)c3cccc4c3oc3ccccc34)cccc21 ; CC1(C)CCC(C)(C)c2c(N(c3ccc4c(c3)oc3ccccc34)c3cccc4c3-c3ccccc3C43C4CC5CC(C4)CC3C5)cccc21 ; CC1(C)CCC(C)(C)c2c(N(c3ccc4oc5ccccc5c4c3)c3cccc4c3-c3ccccc3C43C4CC5CC(C4)CC3C5)cccc21]

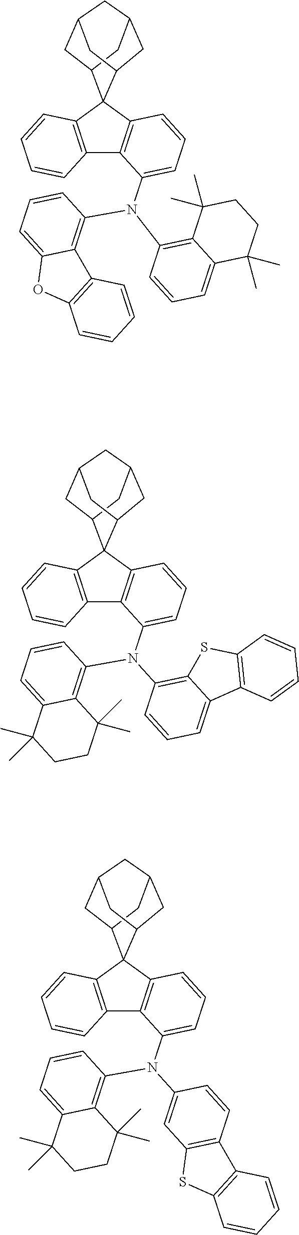

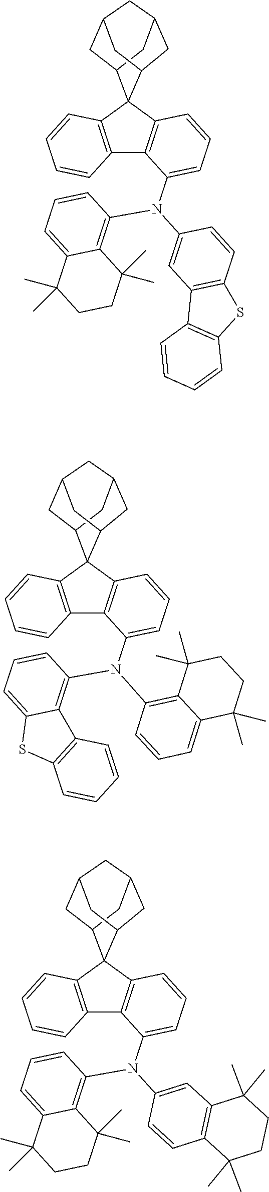

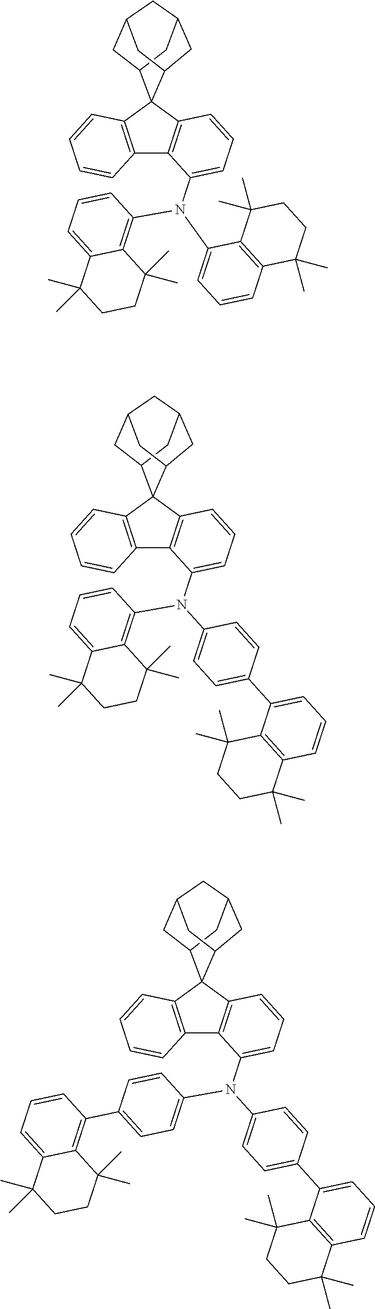

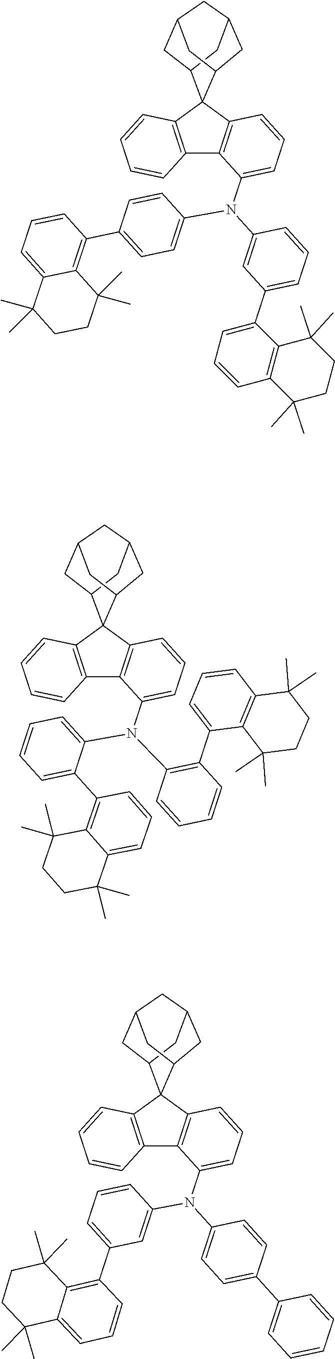

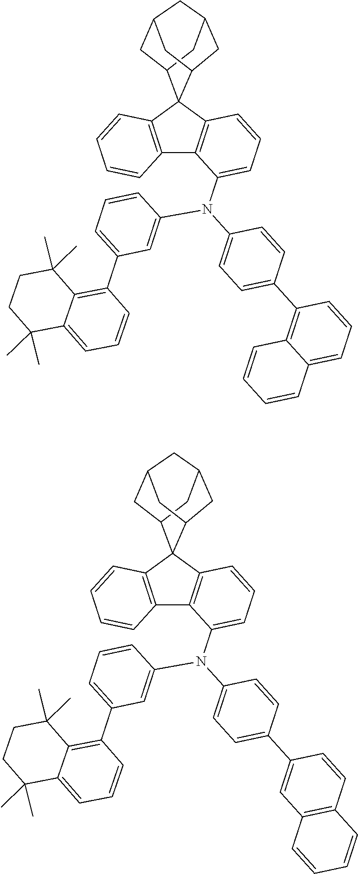 [SMILES: CC1(C)CCC(C)(C)c2c(-c3cccc(N(c4ccc(-c5cccc6ccccc56)cc4)c4cccc5c4-c4ccccc4C54C5CC6CC(C5)CC4C6)c3)cccc21 ; CC1(C)CCC(C)(C)c2c(-c3cccc(N(c4ccc(-c5ccc6ccccc6c5)cc4)c4cccc5c4-c4ccccc4C54C5CC6CC(C5)CC4C6)c3)cccc21]

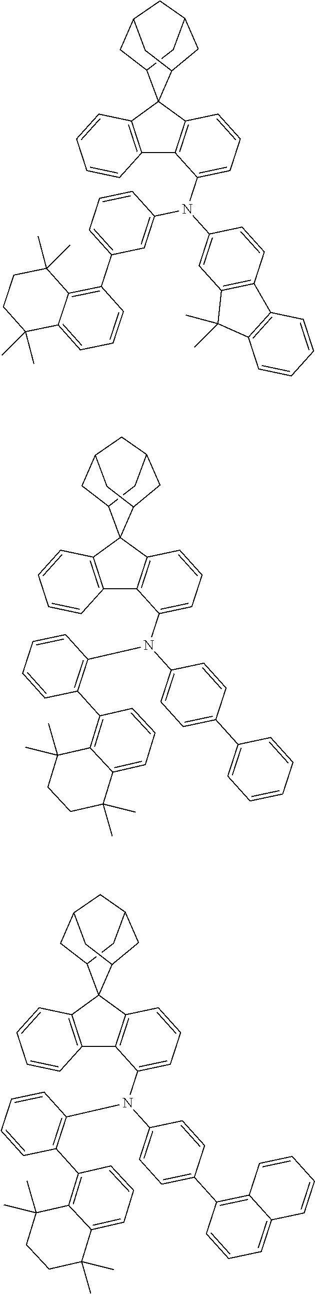 [SMILES: CC1(C)CCC(C)(C)c2c(-c3cccc(N(c4ccc5c(c4)C(C)(C)c4ccccc4-5)c4cccc5c4-c4ccccc4C54C5CC6CC(C5)CC4C6)c3)cccc21 ; CC1(C)CCC(C)(C)c2c(-c3ccccc3N(c3ccc(-c4ccccc4)cc3)c3cccc4c3-c3ccccc3C43C4CC5CC(C4)CC3C5)cccc21 ; CC1(C)CCC(C)(C)c2c(-c3ccccc3N(c3ccc(-c4cccc5ccccc45)cc3)c3cccc4c3-c3ccccc3C43C4CC5CC(C4)CC3C5)cccc21]

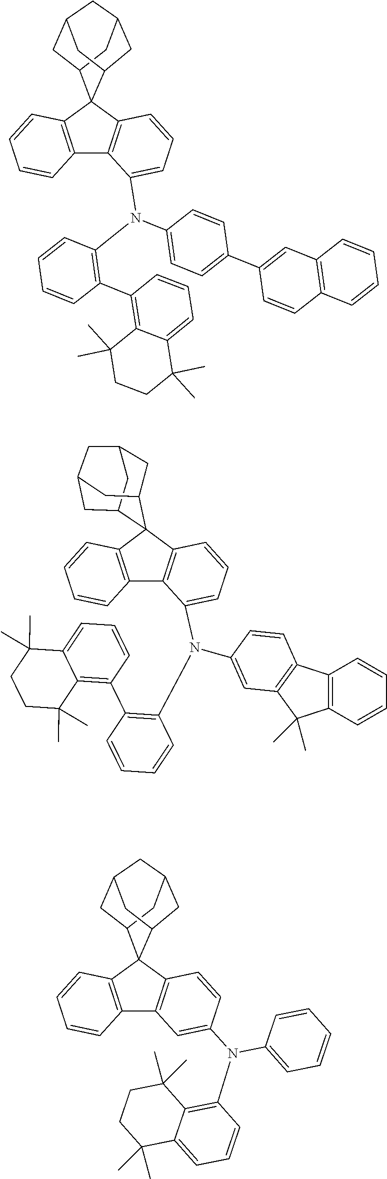 [SMILES: CC1(C)CCC(C)(C)c2c(-c3ccccc3N(c3ccc(-c4ccc5ccccc5c4)cc3)c3cccc4c3-c3ccccc3C43C4CC5CC(C4)CC3C5)cccc21 ; CC1(C)CCC(C)(C)c2c(-c3ccccc3N(c3ccc4c(c3)C(C)(C)c3ccccc3-4)c3cccc4c3-c3ccccc3C43C4CC5CC(C4)CC3C5)cccc21 ; CC1(C)CCC(C)(C)c2c(N(c3ccccc3)c3ccc4c(c3)-c3ccccc3C43C4CC5CC(C4)CC3C5)cccc21]

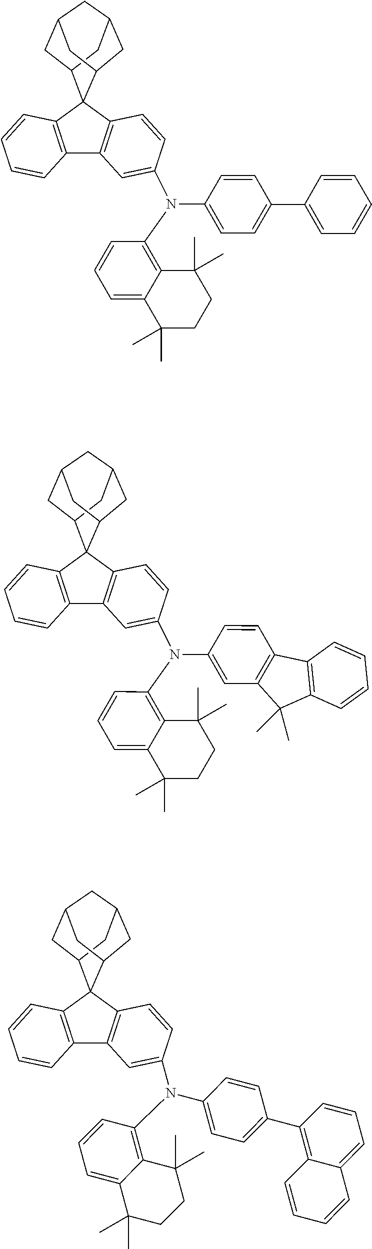 [SMILES: CC1(C)CCC(C)(C)c2c(N(c3ccc(-c4ccccc4)cc3)c3ccc4c(c3)-c3ccccc3C43C4CC5CC(C4)CC3C5)cccc21 ; CC1(C)CCC(C)(C)c2c(N(c3ccc4c(c3)-c3ccccc3C43C4CC5CC(C4)CC3C5)c3ccc4c(c3)C(C)(C)c3ccccc3-4)cccc21 ; CC1(C)CCC(C)(C)c2c(N(c3ccc(-c4cccc5ccccc45)cc3)c3ccc4c(c3)-c3ccccc3C43C4CC5CC(C4)CC3C5)cccc21]

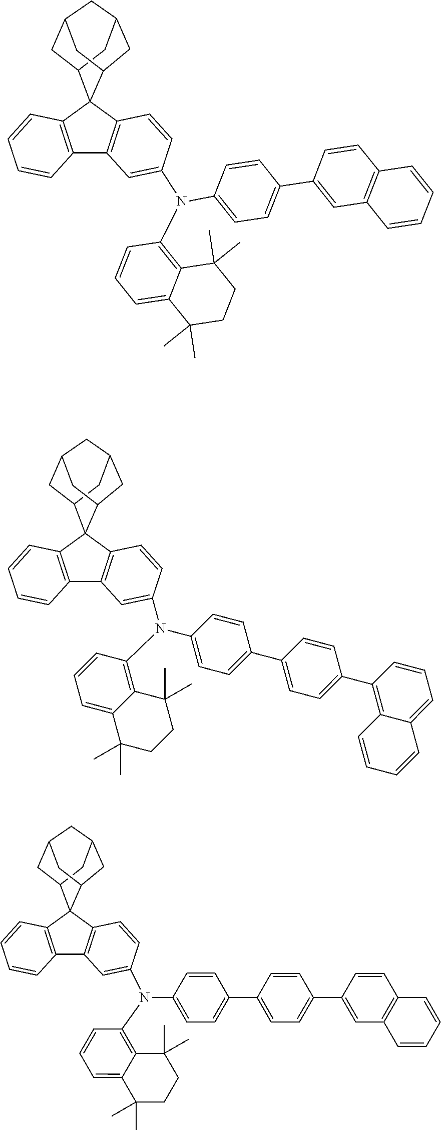 [SMILES: CC1(C)CCC(C)(C)c2c(N(c3ccc(-c4ccc5ccccc5c4)cc3)c3ccc4c(c3)-c3ccccc3C43C4CC5CC(C4)CC3C5)cccc21 ; CC1(C)CCC(C)(C)c2c(N(c3ccc(-c4ccc(-c5cccc6ccccc56)cc4)cc3)c3ccc4c(c3)-c3ccccc3C43C4CC5CC(C4)CC3C5)cccc21 ; CC1(C)CCC(C)(C)c2c(N(c3ccc(-c4ccc(-c5ccc6ccccc6c5)cc4)cc3)c3ccc4c(c3)-c3ccccc3C43C4CC5CC(C4)CC3C5)cccc21]

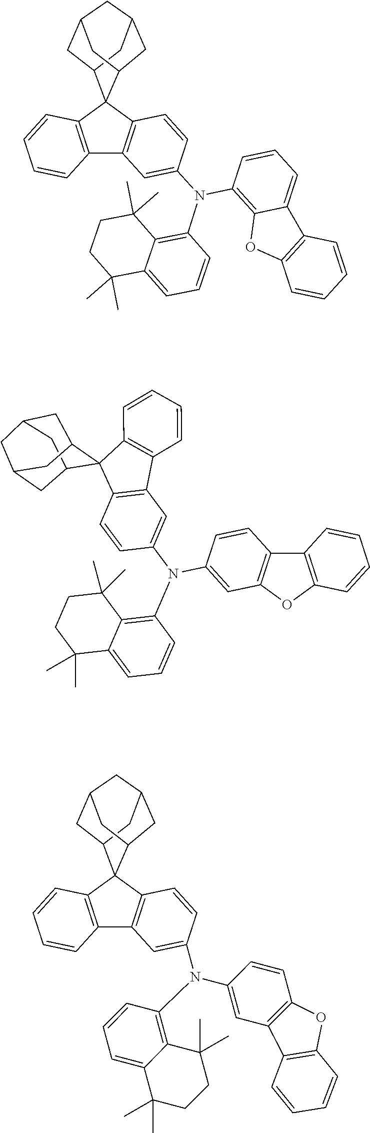 [SMILES: CC1(C)CCC(C)(C)c2c(N(c3ccc4c(c3)-c3ccccc3C43C4CC5CC(C4)CC3C5)c3cccc4c3oc3ccccc34)cccc21 ; CC1(C)CCC(C)(C)c2c(N(c3ccc4c(c3)-c3ccccc3C43C4CC5CC(C4)CC3C5)c3ccc4c(c3)oc3ccccc34)cccc21 ; CC1(C)CCC(C)(C)c2c(N(c3ccc4c(c3)-c3ccccc3C43C4CC5CC(C4)CC3C5)c3ccc4oc5ccccc5c4c3)cccc21]

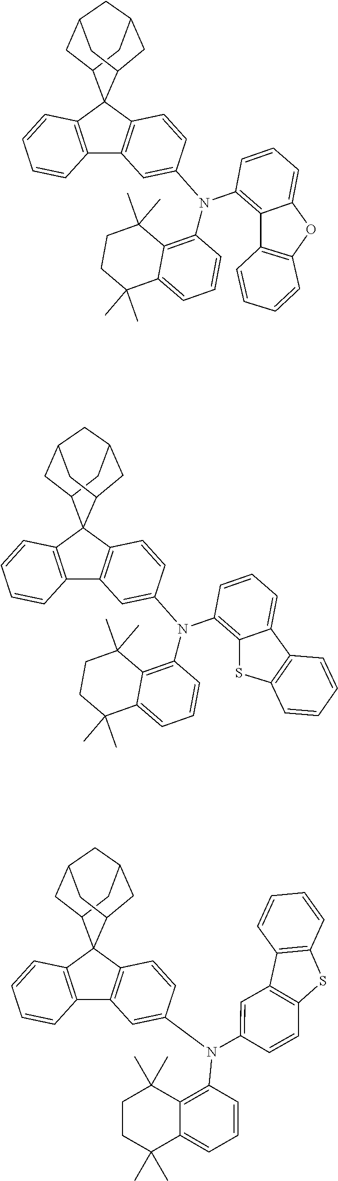 [SMILES: CC1(C)CCC(C)(C)c2c(N(c3ccc4c(c3)-c3ccccc3C43C4CC5CC(C4)CC3C5)c3cccc4oc5ccccc5c34)cccc21 ; CC1(C)CCC(C)(C)c2c(N(c3ccc4c(c3)-c3ccccc3C43C4CC5CC(C4)CC3C5)c3cccc4c3sc3ccccc34)cccc21 ; CC1(C)CCC(C)(C)c2c(N(c3ccc4c(c3)-c3ccccc3C43C4CC5CC(C4)CC3C5)c3ccc4sc5ccccc5c4c3)cccc21]

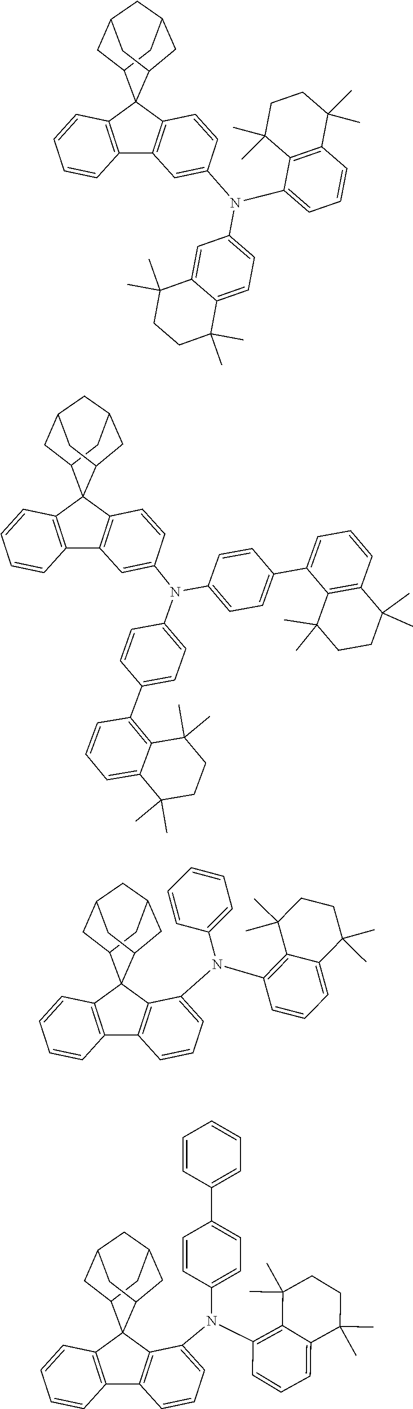

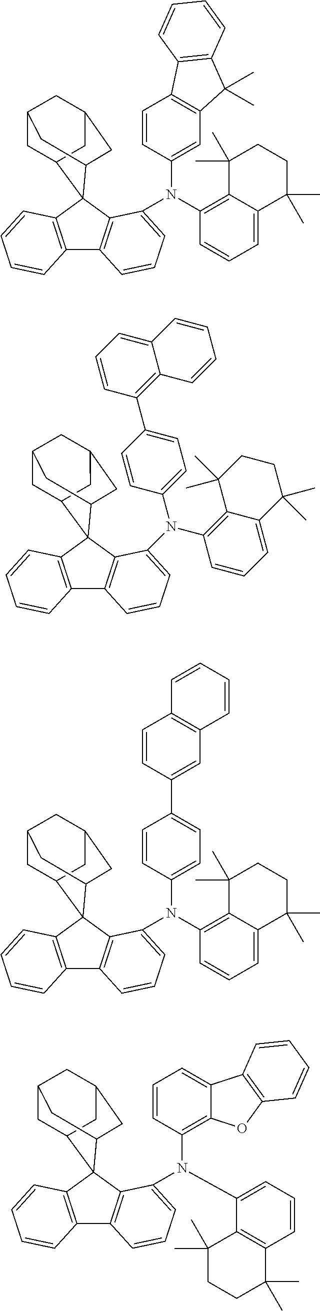 [SMILES: CC1(C)CCC(C)(C)c2c(N(c3ccc4c(c3)C(C)(C)c3ccccc3-4)c3cccc4c3C3(c5ccccc5-4)C4CC5CC(C4)CC3C5)cccc21 ; CC1(C)CCC(C)(C)c2c(N(c3ccc(-c4cccc5ccccc45)cc3)c3cccc4c3C3(c5ccccc5-4)C4CC5CC(C4)CC3C5)cccc21 ; CC1(C)CCC(C)(C)c2c(N(c3ccc(-c4ccc5ccccc5c4)cc3)c3cccc4c3C3(c5ccccc5-4)C4CC5CC(C4)CC3C5)cccc21 ; CC1(C)CCC(C)(C)c2c(N(c3cccc4c3C3(c5ccccc5-4)C4CC5CC(C4)CC3C5)c3cccc4c3oc3ccccc34)cccc21]

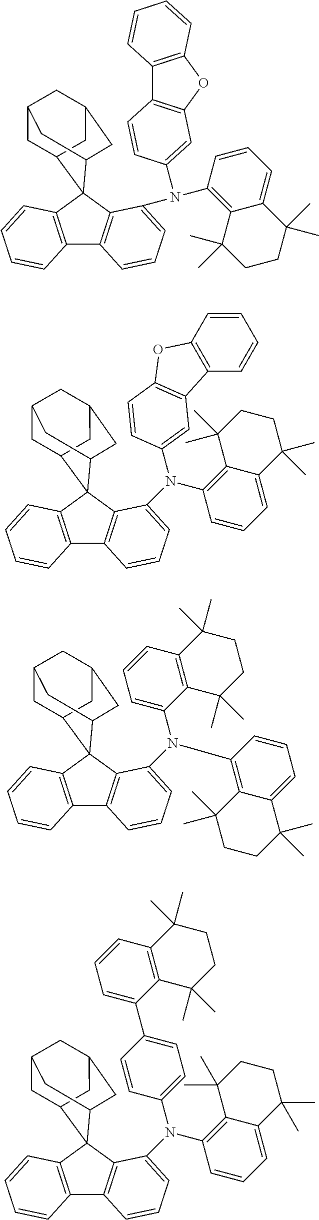

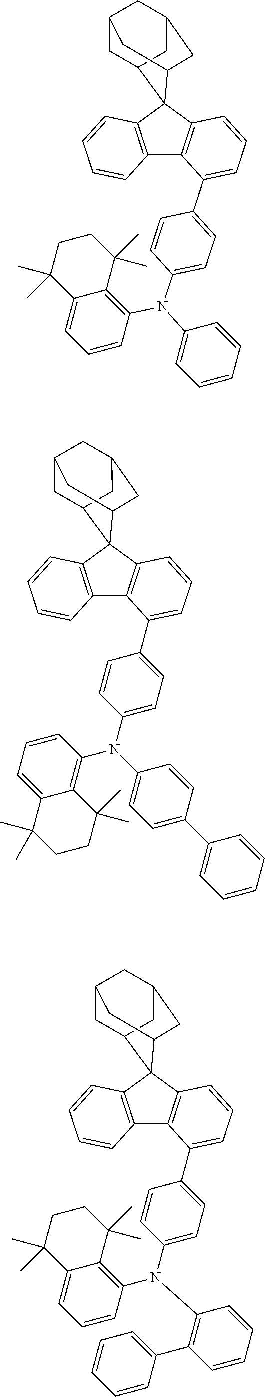 [SMILES: CC1(C)CCC(C)(C)c2c(N(c3ccccc3)c3ccc(-c4cccc5c4-c4ccccc4C54C5CC6CC(C5)CC4C6)cc3)cccc21 ; CC1(C)CCC(C)(C)c2c(N(c3ccc(-c4ccccc4)cc3)c3ccc(-c4cccc5c4-c4ccccc4C54C5CC6CC(C5)CC4C6)cc3)cccc21 ; CC1(C)CCC(C)(C)c2c(N(c3ccc(-c4cccc5c4-c4ccccc4C54C5CC6CC(C5)CC4C6)cc3)c3ccccc3-c3ccccc3)cccc21]

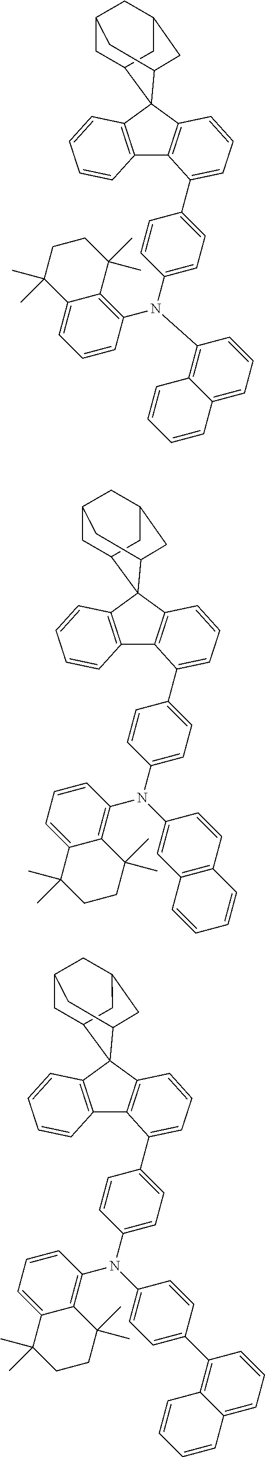 [SMILES: CC1(C)CCC(C)(C)c2c(N(c3ccc(-c4cccc5c4-c4ccccc4C54C5CC6CC(C5)CC4C6)cc3)c3cccc4ccccc34)cccc21 ; CC1(C)CCC(C)(C)c2c(N(c3ccc(-c4cccc5c4-c4ccccc4C54C5CC6CC(C5)CC4C6)cc3)c3ccc4ccccc4c3)cccc21 ; CC1(C)CCC(C)(C)c2c(N(c3ccc(-c4cccc5c4-c4ccccc4C54C5CC6CC(C5)CC4C6)cc3)c3ccc(-c4cccc5ccccc45)cc3)cccc21]

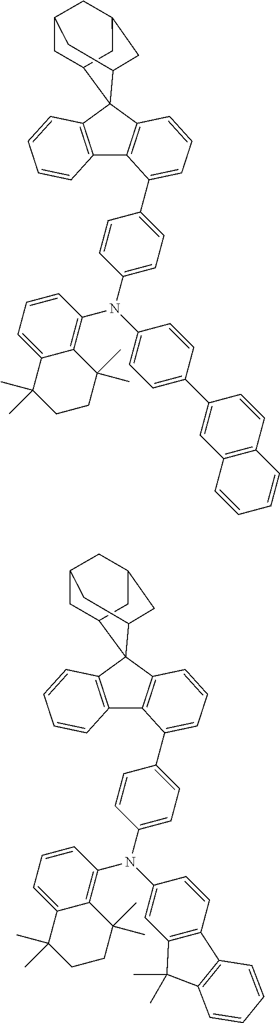 [SMILES: CC1(C)CCC(C)(C)c2c(N(c3ccc(-c4ccc5ccccc5c4)cc3)c3ccc(-c4cccc5c4-c4ccccc4C54C5CC6CC(C5)CC4C6)cc3)cccc21 ; CC1(C)CCC(C)(C)c2c(N(c3ccc(-c4cccc5c4-c4ccccc4C54C5CC6CC(C5)CC4C6)cc3)c3ccc4c(c3)C(C)(C)c3ccccc3-4)cccc21]

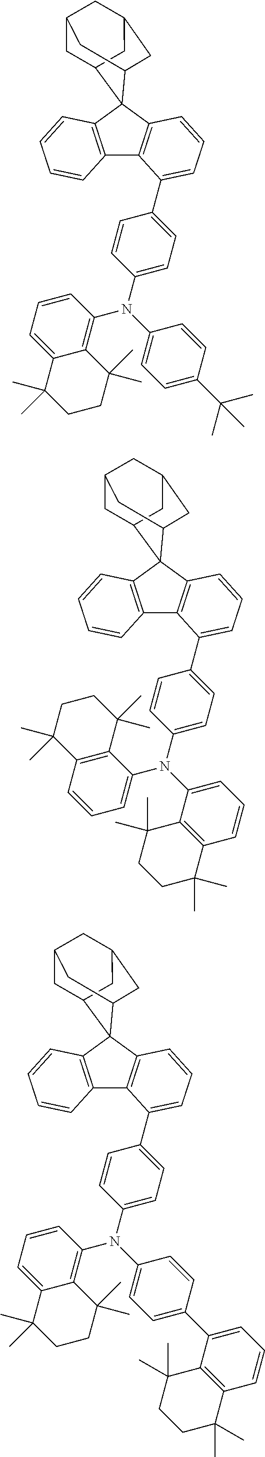

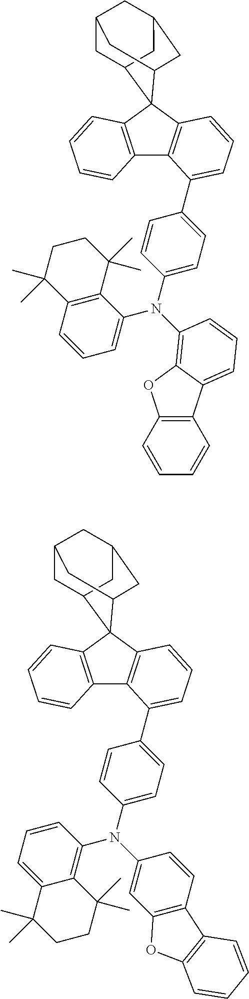 [SMILES: CC1(C)CCC(C)(C)c2c(N(c3ccc(-c4cccc5c4-c4ccccc4C54C5CC6CC(C5)CC4C6)cc3)c3cccc4c3oc3ccccc34)cccc21 ; CC1(C)CCC(C)(C)c2c(N(c3ccc(-c4cccc5c4-c4ccccc4C54C5CC6CC(C5)CC4C6)cc3)c3ccc4c(c3)oc3ccccc34)cccc21]

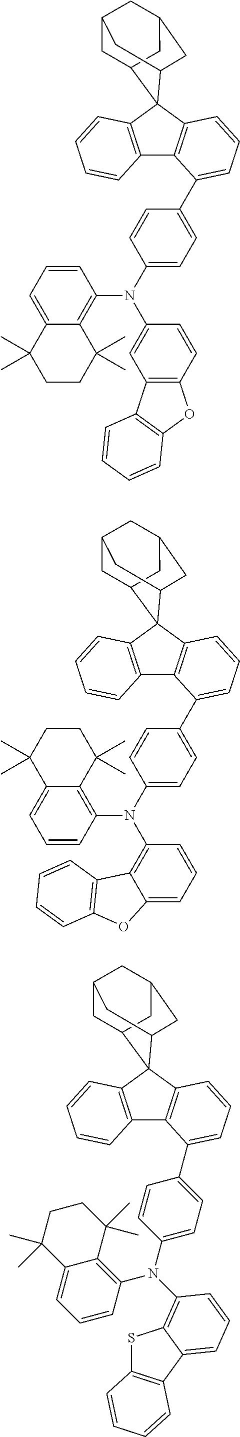

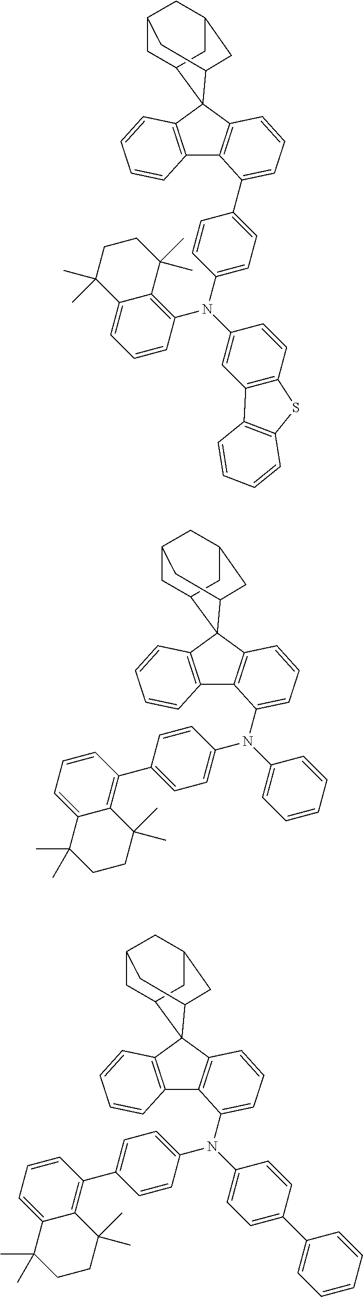 [SMILES: CC1(C)CCC(C)(C)c2c(N(c3ccc(-c4cccc5c4-c4ccccc4C54C5CC6CC(C5)CC4C6)cc3)c3ccc4sc5ccccc5c4c3)cccc21 ; CC1(C)CCC(C)(C)c2c(-c3ccc(N(c4ccccc4)c4cccc5c4-c4ccccc4C54C5CC6CC(C5)CC4C6)cc3)cccc21 ; CC1(C)CCC(C)(C)c2c(-c3ccc(N(c4ccc(-c5ccccc5)cc4)c4cccc5c4-c4ccccc4C54C5CC6CC(C5)CC4C6)cc3)cccc21]

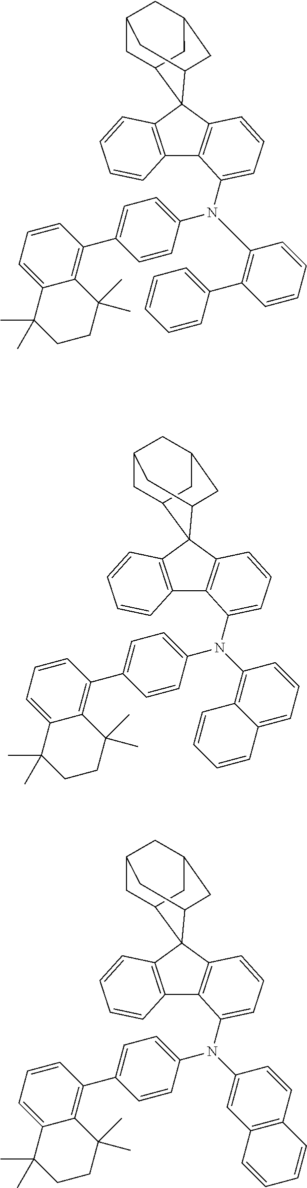 [SMILES: CC1(C)CCC(C)(C)c2c(-c3ccc(N(c4ccccc4-c4ccccc4)c4cccc5c4-c4ccccc4C54C5CC6CC(C5)CC4C6)cc3)cccc21 ; CC1(C)CCC(C)(C)c2c(-c3ccc(N(c4cccc5c4-c4ccccc4C54C5CC6CC(C5)CC4C6)c4cccc5ccccc45)cc3)cccc21 ; CC1(C)CCC(C)(C)c2c(-c3ccc(N(c4ccc5ccccc5c4)c4cccc5c4-c4ccccc4C54C5CC6CC(C5)CC4C6)cc3)cccc21]

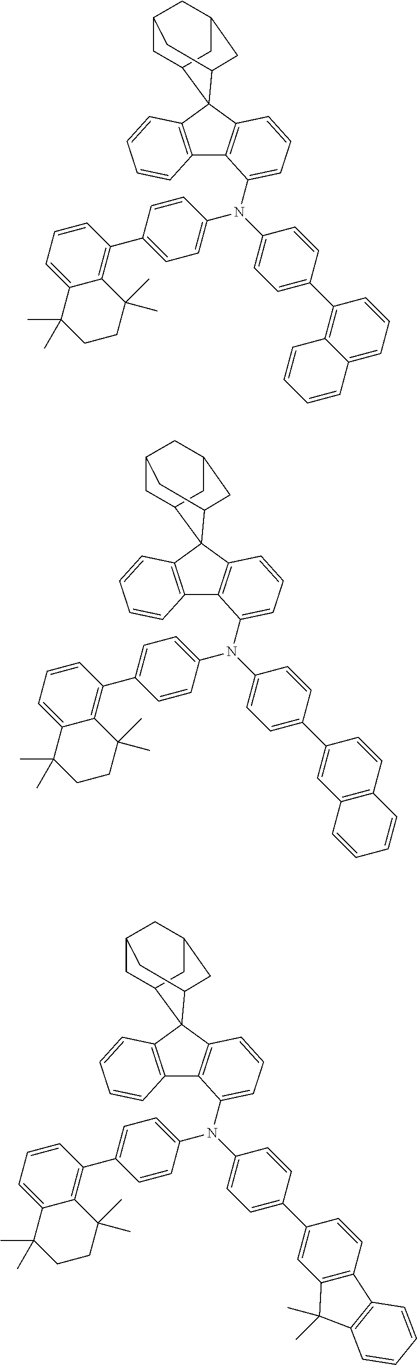 [SMILES: CC1(C)CCC(C)(C)c2c(-c3ccc(N(c4ccc(-c5cccc6ccccc56)cc4)c4cccc5c4-c4ccccc4C54C5CC6CC(C5)CC4C6)cc3)cccc21 ; CC1(C)CCC(C)(C)c2c(-c3ccc(N(c4ccc(-c5ccc6ccccc6c5)cc4)c4cccc5c4-c4ccccc4C54C5CC6CC(C5)CC4C6)cc3)cccc21 ; CC1(C)CCC(C)(C)c2c(-c3ccc(N(c4ccc(-c5ccc6c(c5)C(C)(C)c5ccccc5-6)cc4)c4cccc5c4-c4ccccc4C54C5CC6CC(C5)CC4C6)cc3)cccc21]

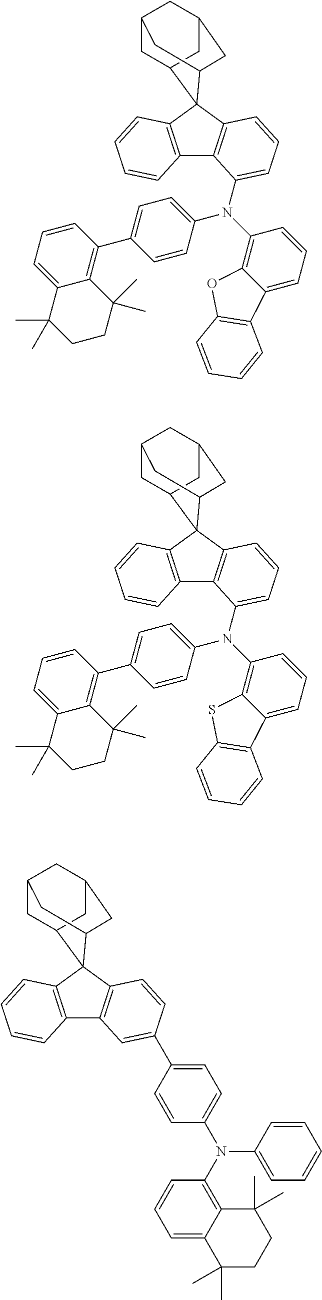 [SMILES: CC1(C)CCC(C)(C)c2c(-c3ccc(N(c4cccc5c4-c4ccccc4C54C5CC6CC(C5)CC4C6)c4cccc5c4oc4ccccc45)cc3)cccc21 ; CC1(C)CCC(C)(C)c2c(-c3ccc(N(c4cccc5c4-c4ccccc4C54C5CC6CC(C5)CC4C6)c4cccc5c4sc4ccccc45)cc3)cccc21 ; CC1(C)CCC(C)(C)c2c(N(c3ccccc3)c3ccc(-c4ccc5c(c4)-c4ccccc4C54C5CC6CC(C5)CC4C6)cc3)cccc21]

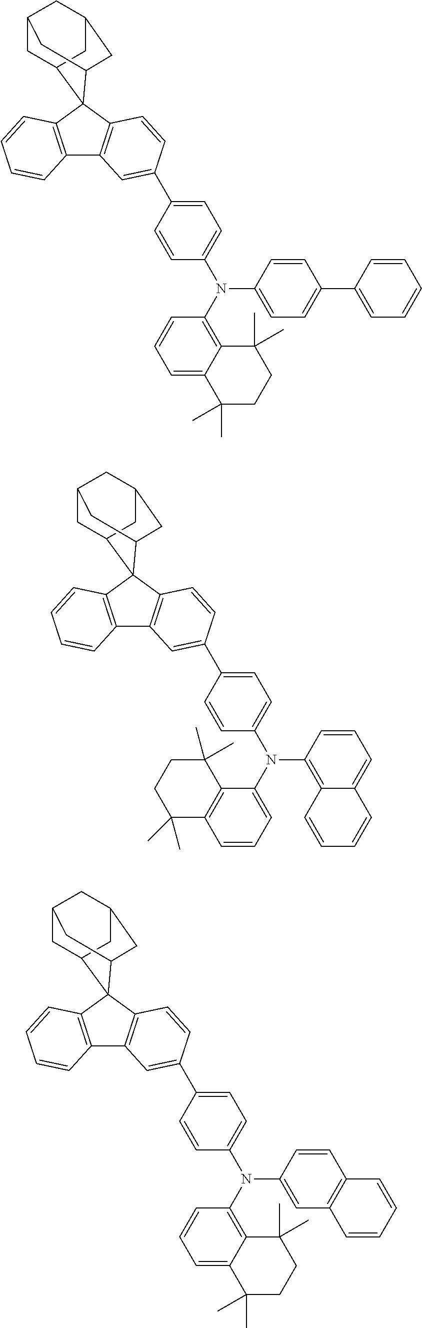 [SMILES: CC1(C)CCC(C)(C)c2c(N(c3ccc(-c4ccccc4)cc3)c3ccc(-c4ccc5c(c4)-c4ccccc4C54C5CC6CC(C5)CC4C6)cc3)cccc21 ; CC1(C)CCC(C)(C)c2c(N(c3ccc(-c4ccc5c(c4)-c4ccccc4C54C5CC6CC(C5)CC4C6)cc3)c3cccc4ccccc34)cccc21 ; CC1(C)CCC(C)(C)c2c(N(c3ccc(-c4ccc5c(c4)-c4ccccc4C54C5CC6CC(C5)CC4C6)cc3)c3ccc4ccccc4c3)cccc21]

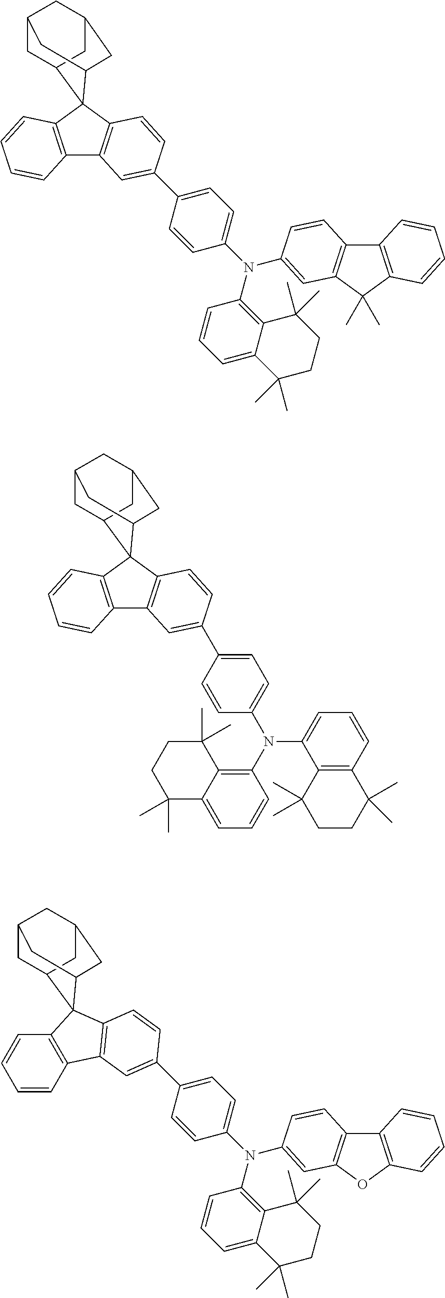 [SMILES: CC1(C)CCC(C)(C)c2c(N(c3ccc(-c4ccc5c(c4)-c4ccccc4C54C5CC6CC(C5)CC4C6)cc3)c3ccc4c(c3)C(C)(C)c3ccccc3-4)cccc21 ; CC1(C)CCC(C)(C)c2c(N(c3ccc(-c4ccc5c(c4)-c4ccccc4C54C5CC6CC(C5)CC4C6)cc3)c3cccc4c3C(C)(C)CCC4(C)C)cccc21 ; CC1(C)CCC(C)(C)c2c(N(c3ccc(-c4ccc5c(c4)-c4ccccc4C54C5CC6CC(C5)CC4C6)cc3)c3ccc4c(c3)oc3ccccc34)cccc21]

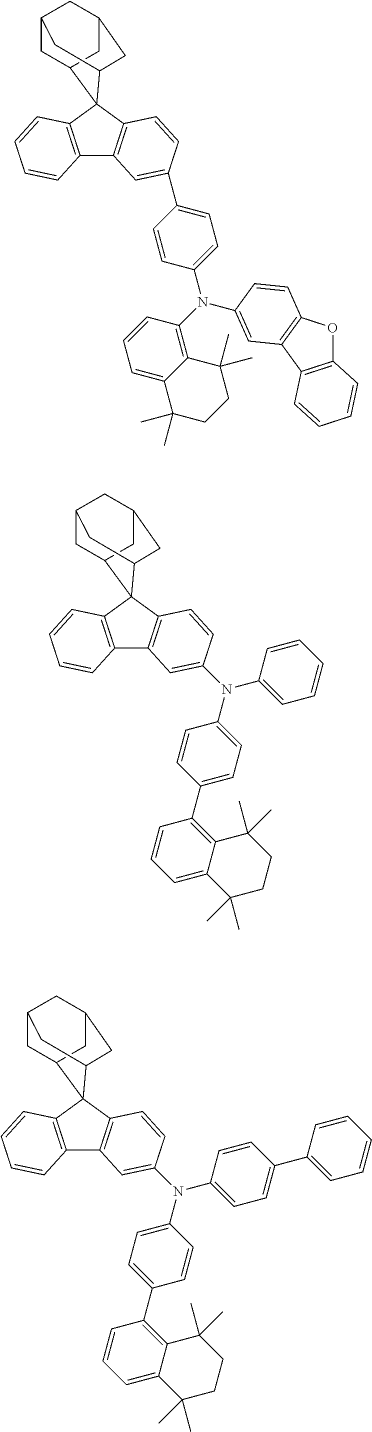 [SMILES: CC1(C)CCC(C)(C)c2c(N(c3ccc(-c4ccc5c(c4)-c4ccccc4C54C5CC6CC(C5)CC4C6)cc3)c3ccc4oc5ccccc5c4c3)cccc21 ; CC1(C)CCC(C)(C)c2c(-c3ccc(N(c4ccccc4)c4ccc5c(c4)-c4ccccc4C54C5CC6CC(C5)CC4C6)cc3)cccc21 ; CC1(C)CCC(C)(C)c2c(-c3ccc(N(c4ccc(-c5ccccc5)cc4)c4ccc5c(c4)-c4ccccc4C54C5CC6CC(C5)CC4C6)cc3)cccc21]

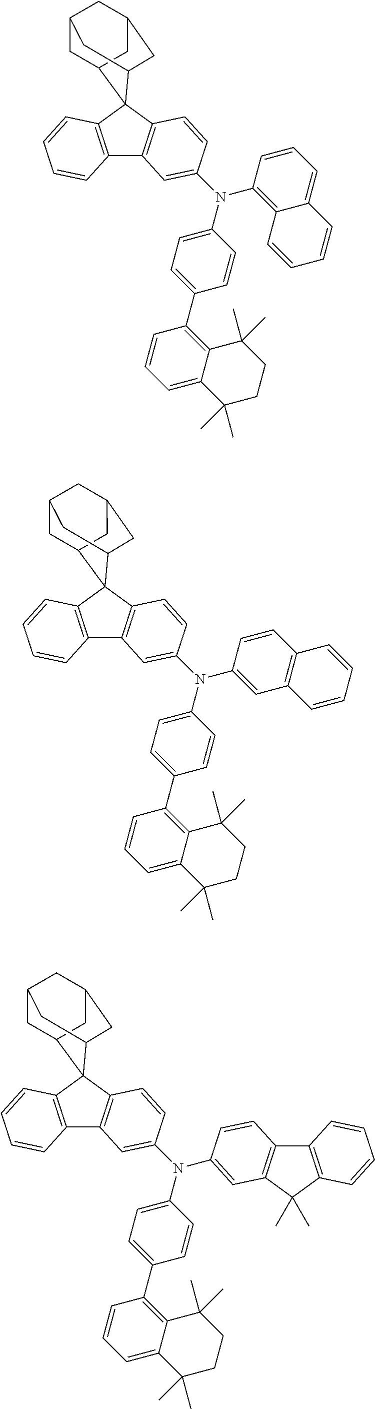 [SMILES: CC1(C)CCC(C)(C)c2c(-c3ccc(N(c4ccc5c(c4)-c4ccccc4C54C5CC6CC(C5)CC4C6)c4cccc5ccccc45)cc3)cccc21 ; CC1(C)CCC(C)(C)c2c(-c3ccc(N(c4ccc5c(c4)-c4ccccc4C54C5CC6CC(C5)CC4C6)c4ccc5ccccc5c4)cc3)cccc21 ; CC1(C)CCC(C)(C)c2c(-c3ccc(N(c4ccc5c(c4)-c4ccccc4C54C5CC6CC(C5)CC4C6)c4ccc5c(c4)C(C)(C)c4ccccc4-5)cc3)cccc21]

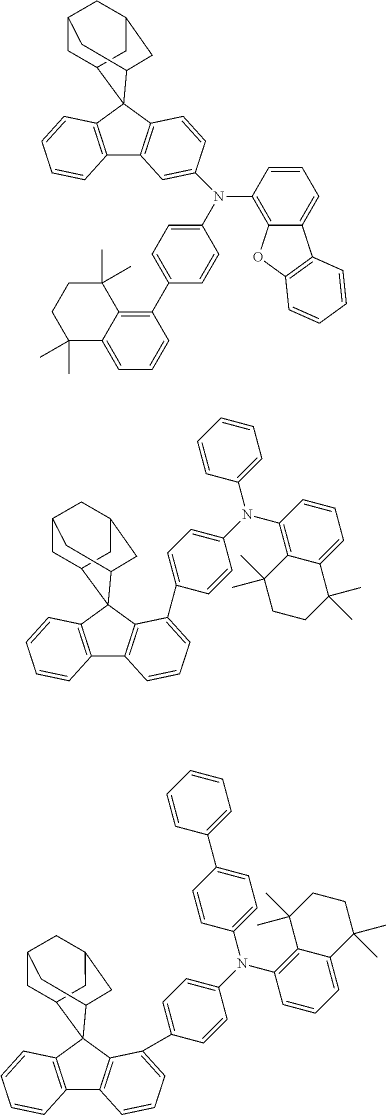 [SMILES: CC1(C)CCC(C)(C)c2c(-c3ccc(N(c4ccc5c(c4)-c4ccccc4C54C5CC6CC(C5)CC4C6)c4cccc5c4oc4ccccc45)cc3)cccc21 ; CC1(C)CCC(C)(C)c2c(N(c3ccccc3)c3ccc(-c4cccc5c4C4(c6ccccc6-5)C5CC6CC(C5)CC4C6)cc3)cccc21 ; CC1(C)CCC(C)(C)c2c(N(c3ccc(-c4ccccc4)cc3)c3ccc(-c4cccc5c4C4(c6ccccc6-5)C5CC6CC(C5)CC4C6)cc3)cccc21]

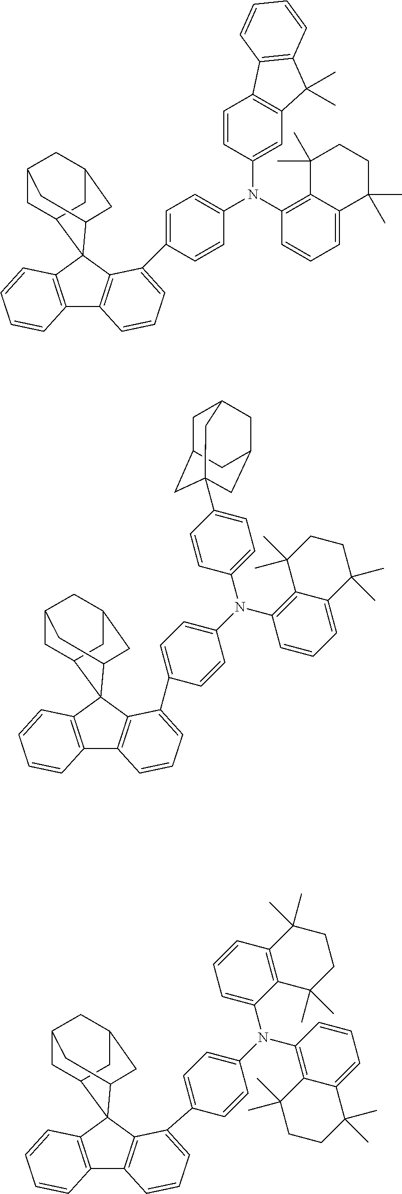 [SMILES: CC1(C)CCC(C)(C)c2c(N(c3ccc(-c4cccc5c4C4(c6ccccc6-5)C5CC6CC(C5)CC4C6)cc3)c3ccc4c(c3)C(C)(C)c3ccccc3-4)cccc21 ; CC1(C)CCC(C)(C)c2c(N(c3ccc(-c4cccc5c4C4(c6ccccc6-5)C5CC6CC(C5)CC4C6)cc3)c3ccc(C45CC6CC(CC(C6)C4)C5)cc3)cccc21 ; CC1(C)CCC(C)(C)c2c(N(c3ccc(-c4cccc5c4C4(c6ccccc6-5)C5CC6CC(C5)CC4C6)cc3)c3cccc4c3C(C)(C)CCC4(C)C)cccc21]

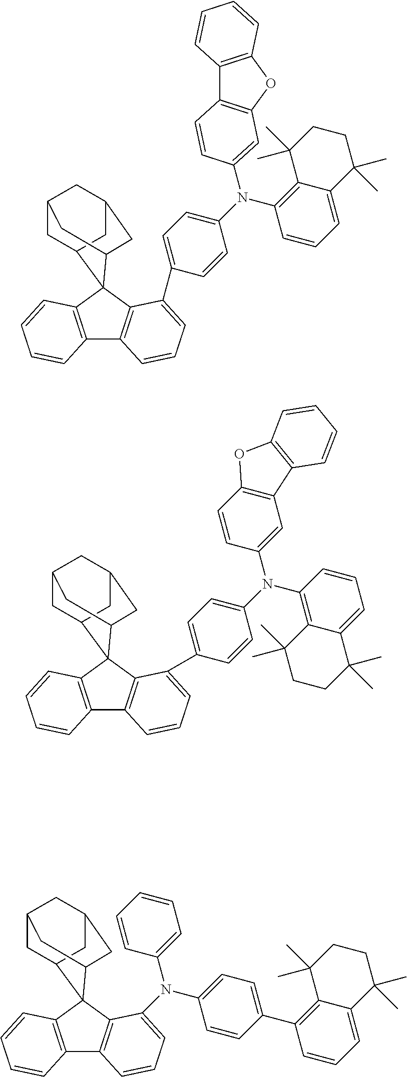 [SMILES: CC1(C)CCC(C)(C)c2c(N(c3ccc(-c4cccc5c4C4(c6ccccc6-5)C5CC6CC(C5)CC4C6)cc3)c3ccc4c(c3)oc3ccccc34)cccc21 ; CC1(C)CCC(C)(C)c2c(N(c3ccc(-c4cccc5c4C4(c6ccccc6-5)C5CC6CC(C5)CC4C6)cc3)c3ccc4oc5ccccc5c4c3)cccc21 ; CC1(C)CCC(C)(C)c2c(-c3ccc(N(c4ccccc4)c4cccc5c4C4(c6ccccc6-5)C5CC6CC(C5)CC4C6)cc3)cccc21]

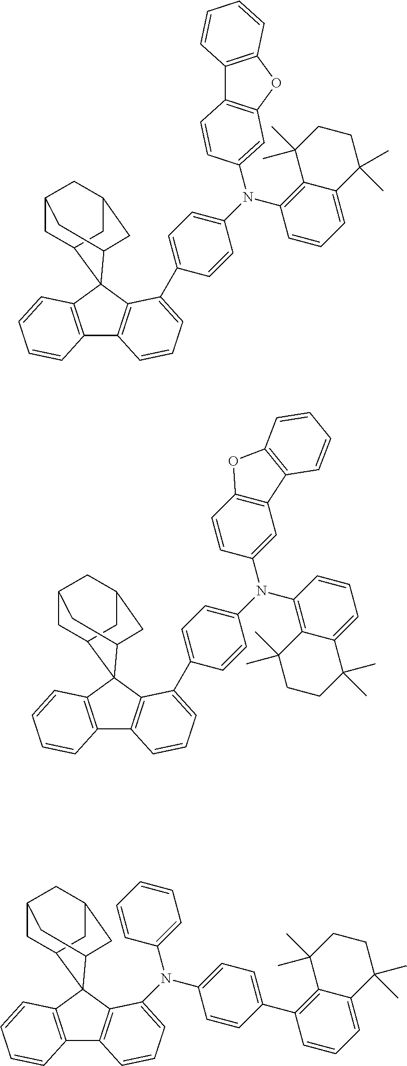 [SMILES: CC1(C)CCC(C)(C)c2c(N(c3ccc(-c4cccc5c4C4(c6ccccc6-5)C5CC6CC(C5)CC4C6)cc3)c3ccc4c(c3)oc3ccccc34)cccc21 ; CC1(C)CCC(C)(C)c2c(N(c3ccc(-c4cccc5c4C4(c6ccccc6-5)C5CC6CC(C5)CC4C6)cc3)c3ccc4oc5ccccc5c4c3)cccc21 ; CC1(C)CCC(C)(C)c2c(-c3ccc(N(c4ccccc4)c4cccc5c4C4(c6ccccc6-5)C5CC6CC(C5)CC4C6)cc3)cccc21]

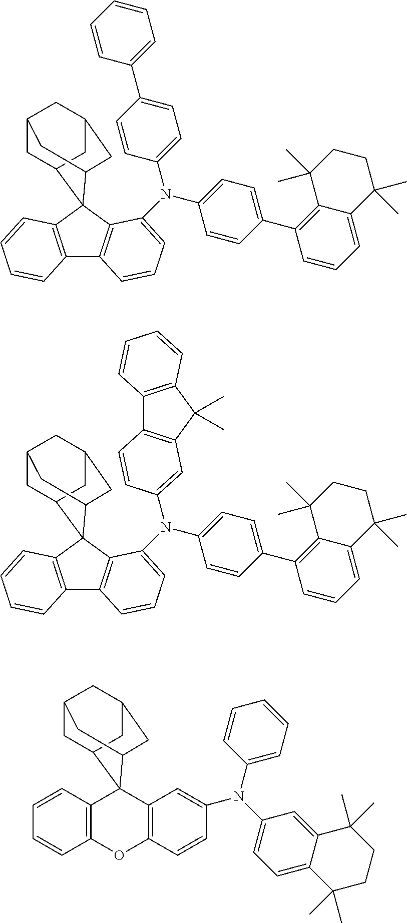 [SMILES: CC1(C)CCC(C)(C)c2c(-c3ccc(N(c4ccc(-c5ccccc5)cc4)c4cccc5c4C4(c6ccccc6-5)C5CC6CC(C5)CC4C6)cc3)cccc21 ; CC1(C)CCC(C)(C)c2c(-c3ccc(N(c4ccc5c(c4)C(C)(C)c4ccccc4-5)c4cccc5c4C4(c6ccccc6-5)C5CC6CC(C5)CC4C6)cc3)cccc21 ; CC1(C)CCC(C)(C)c2cc(N(c3ccccc3)c3ccc4c(c3)C3(c5ccccc5O4)C4CC5CC(C4)CC3C5)ccc21]

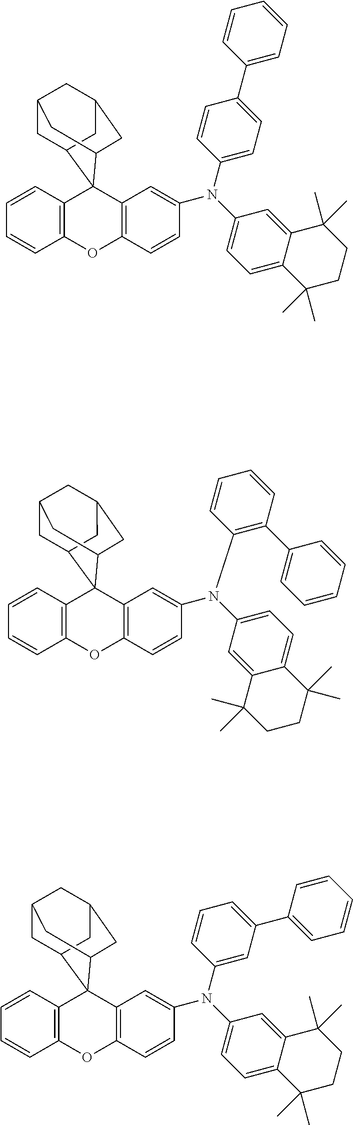 [SMILES: CC1(C)CCC(C)(C)c2cc(N(c3ccc(-c4ccccc4)cc3)c3ccc4c(c3)C3(c5ccccc5O4)C4CC5CC(C4)CC3C5)ccc21 ; CC1(C)CCC(C)(C)c2cc(N(c3ccc4c(c3)C3(c5ccccc5O4)C4CC5CC(C4)CC3C5)c3ccccc3-c3ccccc3)ccc21 ; CC1(C)CCC(C)(C)c2cc(N(c3cccc(-c4ccccc4)c3)c3ccc4c(c3)C3(c5ccccc5O4)C4CC5CC(C4)CC3C5)ccc21]

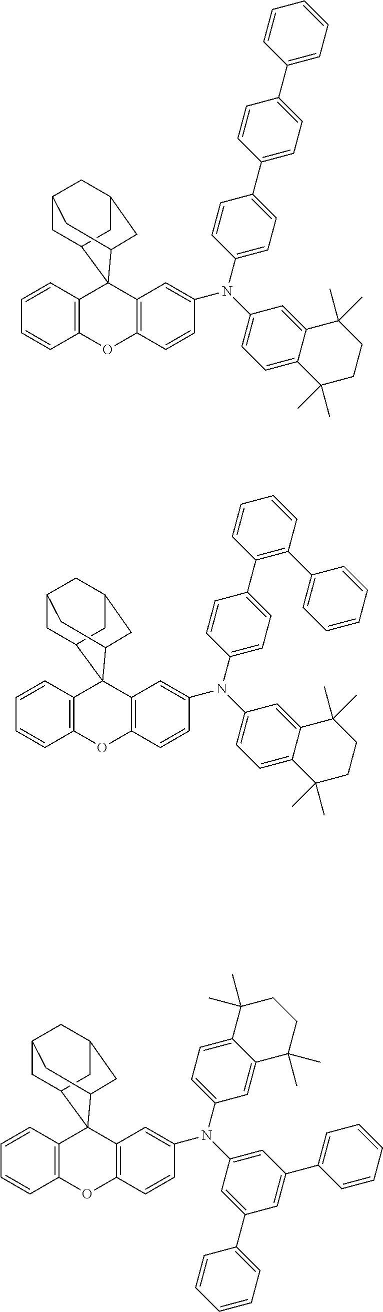 [SMILES: CC1(C)CCC(C)(C)c2cc(N(c3ccc(-c4ccc(-c5ccccc5)cc4)cc3)c3ccc4c(c3)C3(c5ccccc5O4)C4CC5CC(C4)CC3C5)ccc21 ; CC1(C)CCC(C)(C)c2cc(N(c3ccc(-c4ccccc4-c4ccccc4)cc3)c3ccc4c(c3)C3(c5ccccc5O4)C4CC5CC(C4)CC3C5)ccc21 ; CC1(C)CCC(C)(C)c2cc(N(c3cc(-c4ccccc4)cc(-c4ccccc4)c3)c3ccc4c(c3)C3(c5ccccc5O4)C4CC5CC(C4)CC3C5)ccc21]

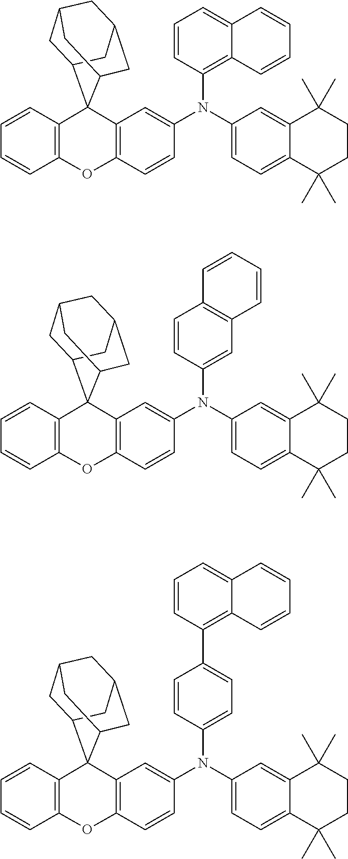 [SMILES: CC1(C)CCC(C)(C)c2cc(N(c3ccc4c(c3)C3(c5ccccc5O4)C4CC5CC(C4)CC3C5)c3cccc4ccccc34)ccc21 ; CC1(C)CCC(C)(C)c2cc(N(c3ccc4c(c3)C3(c5ccccc5O4)C4CC5CC(C4)CC3C5)c3ccc4ccccc4c3)ccc21 ; CC1(C)CCC(C)(C)c2cc(N(c3ccc(-c4cccc5ccccc45)cc3)c3ccc4c(c3)C3(c5ccccc5O4)C4CC5CC(C4)CC3C5)ccc21]

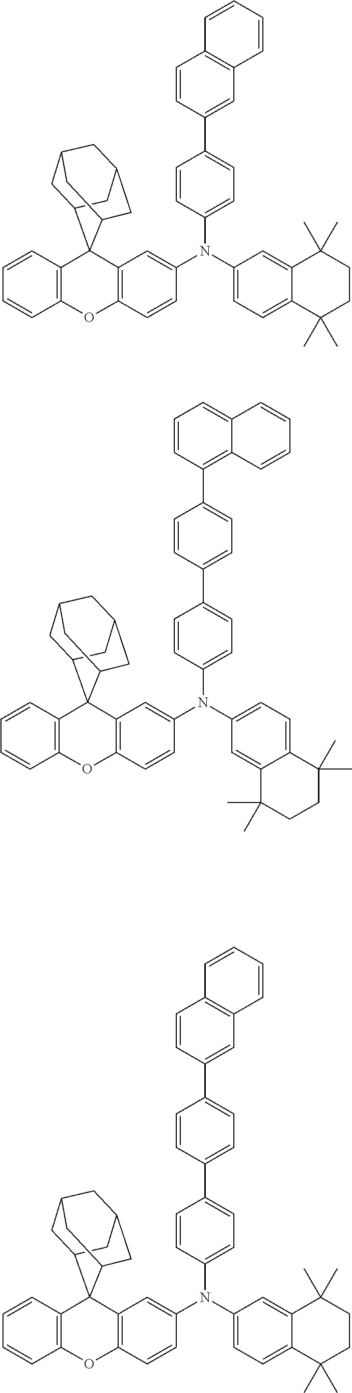 [SMILES: CC1(C)CCC(C)(C)c2cc(N(c3ccc(-c4ccc5ccccc5c4)cc3)c3ccc4c(c3)C3(c5ccccc5O4)C4CC5CC(C4)CC3C5)ccc21 ; CC1(C)CCC(C)(C)c2cc(N(c3ccc(-c4ccc(-c5cccc6ccccc56)cc4)cc3)c3ccc4c(c3)C3(c5ccccc5O4)C4CC5CC(C4)CC3C5)ccc21 ; CC1(C)CCC(C)(C)c2cc(N(c3ccc(-c4ccc(-c5ccc6ccccc6c5)cc4)cc3)c3ccc4c(c3)C3(c5ccccc5O4)C4CC5CC(C4)CC3C5)ccc21]

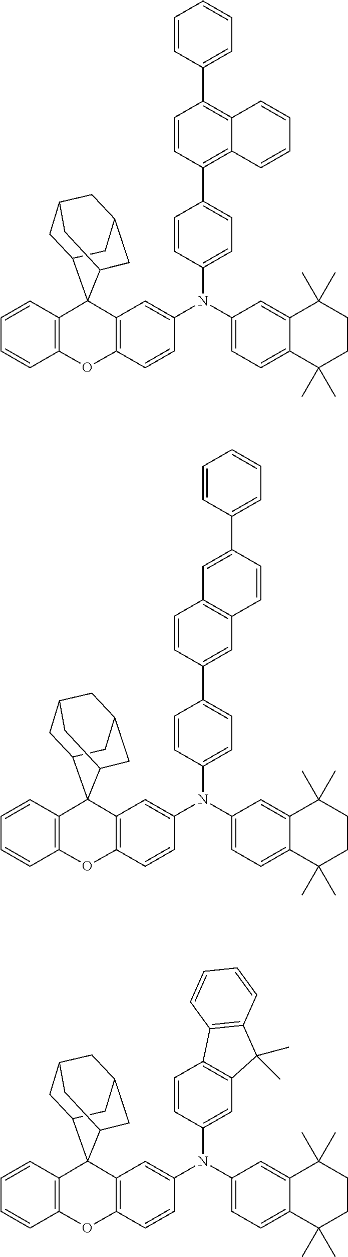 [SMILES: CC1(C)CCC(C)(C)c2cc(N(c3ccc(-c4ccc(-c5ccccc5)c5ccccc45)cc3)c3ccc4c(c3)C3(c5ccccc5O4)C4CC5CC(C4)CC3C5)ccc21 ; CC1(C)CCC(C)(C)c2cc(N(c3ccc(-c4ccc5cc(-c6ccccc6)ccc5c4)cc3)c3ccc4c(c3)C3(c5ccccc5O4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5ccccc5O4)C4CC5CC(C4)CC3C5)ccc21]

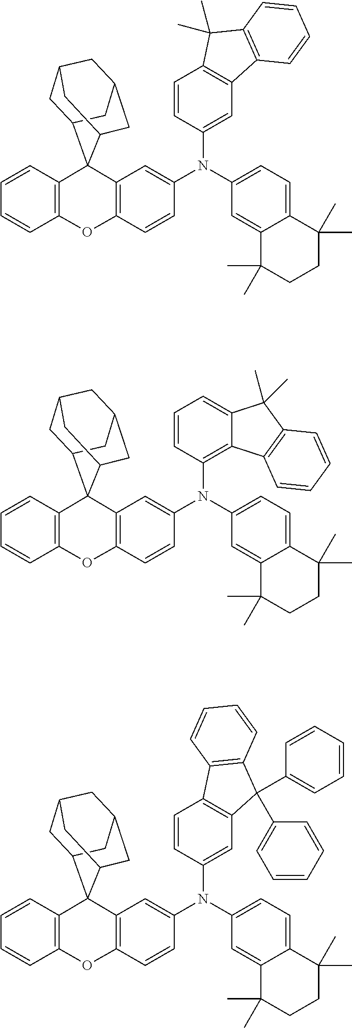 [SMILES: CC1(C)CCC(C)(C)c2cc(N(c3ccc4c(c3)-c3ccccc3C4(C)C)c3ccc4c(c3)C3(c5ccccc5O4)C4CC5CC(C4)CC3C5)ccc21 ; CC1(C)CCC(C)(C)c2cc(N(c3ccc4c(c3)C3(c5ccccc5O4)C4CC5CC(C4)CC3C5)c3cccc4c3-c3ccccc3C4(C)C)ccc21 ; CC1(C)CCC(C)(C)c2cc(N(c3ccc4c(c3)C(c3ccccc3)(c3ccccc3)c3ccccc3-4)c3ccc4c(c3)C3(c5ccccc5O4)C4CC5CC(C4)CC3C5)ccc21]

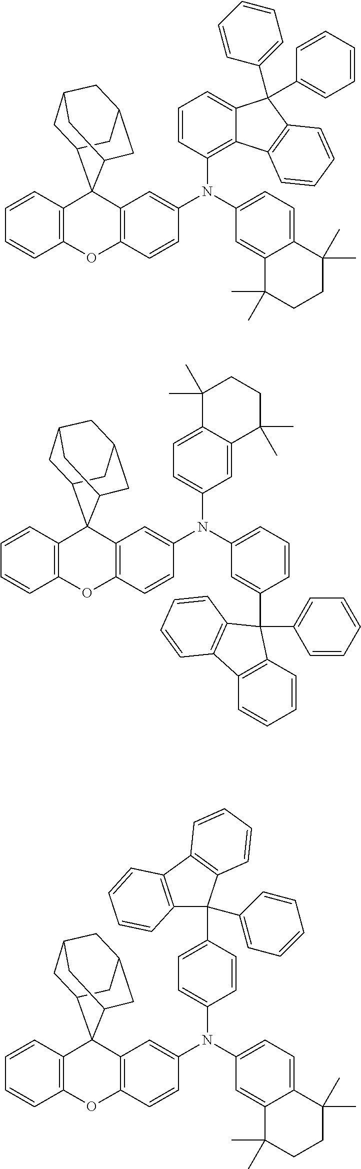 [SMILES: CC1(C)CCC(C)(C)c2cc(N(c3ccc4c(c3)C3(c5ccccc5O4)C4CC5CC(C4)CC3C5)c3cccc4c3-c3ccccc3C4(c3ccccc3)c3ccccc3)ccc21 ; CC1(C)CCC(C)(C)c2cc(N(c3cccc(C4(c5ccccc5)c5ccccc5-c5ccccc54)c3)c3ccc4c(c3)C3(c5ccccc5O4)C4CC5CC(C4)CC3C5)ccc21 ; CC1(C)CCC(C)(C)c2cc(N(c3ccc(C4(c5ccccc5)c5ccccc5-c5ccccc54)cc3)c3ccc4c(c3)C3(c5ccccc5O4)C4CC5CC(C4)CC3C5)ccc21]

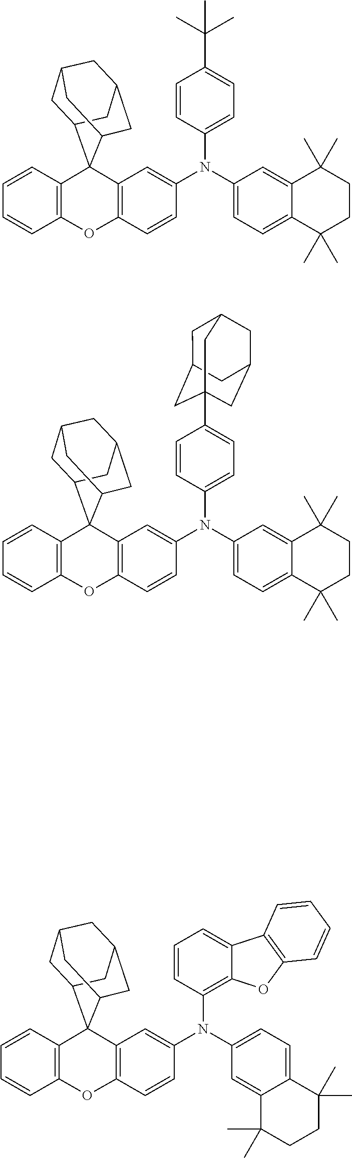 [SMILES: CC(C)(C)c1ccc(N(c2ccc3c(c2)C(C)(C)CCC3(C)C)c2ccc3c(c2)C2(c4ccccc4O3)C3CC4CC(C3)CC2C4)cc1 ; CC1(C)CCC(C)(C)c2cc(N(c3ccc(C45CC6CC(CC(C6)C4)C5)cc3)c3ccc4c(c3)C3(c5ccccc5O4)C4CC5CC(C4)CC3C5)ccc21 ; CC1(C)CCC(C)(C)c2cc(N(c3ccc4c(c3)C3(c5ccccc5O4)C4CC5CC(C4)CC3C5)c3cccc4c3oc3ccccc34)ccc21]

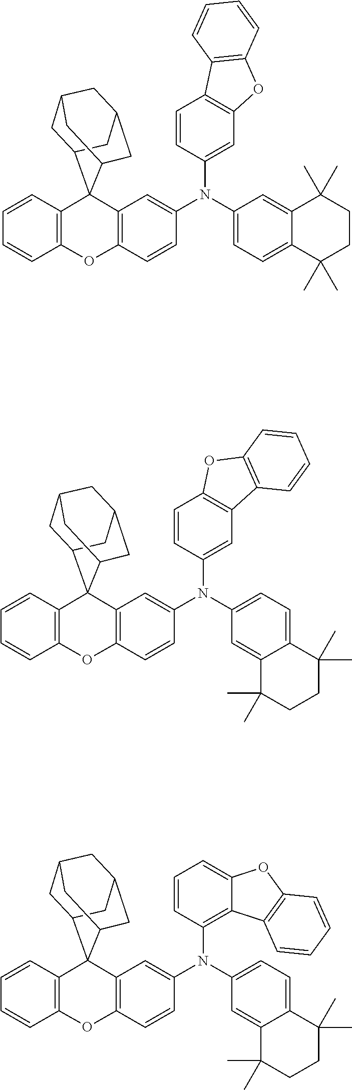 [SMILES: CC1(C)CCC(C)(C)c2cc(N(c3ccc4c(c3)C3(c5ccccc5O4)C4CC5CC(C4)CC3C5)c3ccc4c(c3)oc3ccccc34)ccc21 ; CC1(C)CCC(C)(C)c2cc(N(c3ccc4c(c3)C3(c5ccccc5O4)C4CC5CC(C4)CC3C5)c3ccc4oc5ccccc5c4c3)ccc21 ; CC1(C)CCC(C)(C)c2cc(N(c3ccc4c(c3)C3(c5ccccc5O4)C4CC5CC(C4)CC3C5)c3cccc4oc5ccccc5c34)ccc21]

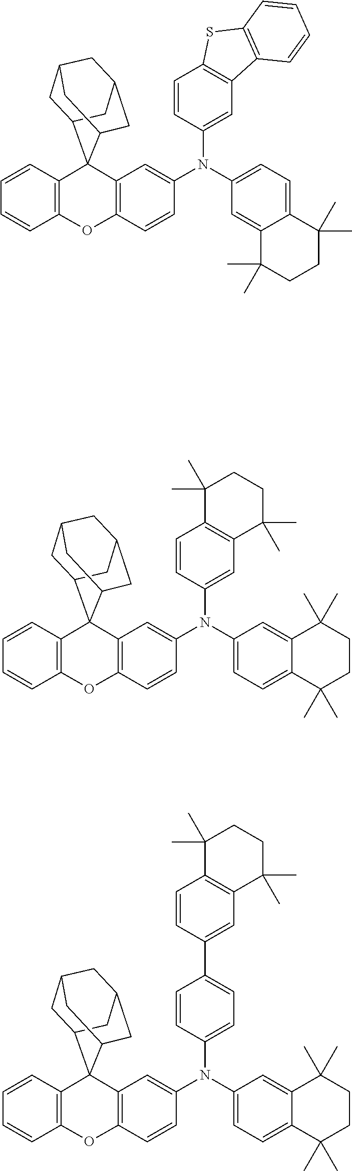 [SMILES: CC1(C)CCC(C)(C)c2cc(N(c3ccc4c(c3)C3(c5ccccc5O4)C4CC5CC(C4)CC3C5)c3ccc4sc5ccccc5c4c3)ccc21 ; CC1(C)CCC(C)(C)c2cc(N(c3ccc4c(c3)C(C)(C)CCC4(C)C)c3ccc4c(c3)C3(c5ccccc5O4)C4CC5CC(C4)CC3C5)ccc21 ; CC1(C)CCC(C)(C)c2cc(-c3ccc(N(c4ccc5c(c4)C(C)(C)CCC5(C)C)c4ccc5c(c4)C4(c6ccccc6O5)C5CC6CC(C5)CC4C6)cc3)ccc21]

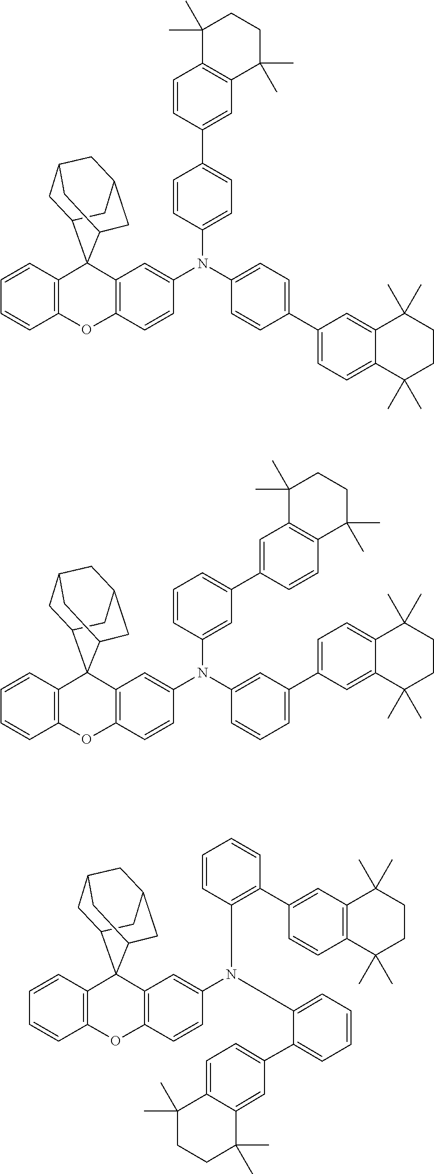 [SMILES: CC1(C)CCC(C)(C)c2cc(-c3ccc(N(c4ccc(-c5ccc6c(c5)C(C)(C)CCC6(C)C)cc4)c4ccc5c(c4)C4(c6ccccc6O5)C5CC6CC(C5)CC4C6)cc3)ccc21 ; CC1(C)CCC(C)(C)c2cc(-c3cccc(N(c4cccc(-c5ccc6c(c5)C(C)(C)CCC6(C)C)c4)c4ccc5c(c4)C4(c6ccccc6O5)C5CC6CC(C5)CC4C6)c3)ccc21 ; CC1(C)CCC(C)(C)c2cc(-c3ccccc3N(c3ccc4c(c3)C3(c5ccccc5O4)C4CC5CC(C4)CC3C5)c3ccccc3-c3ccc4c(c3)C(C)(C)CCC4(C)C)ccc21]

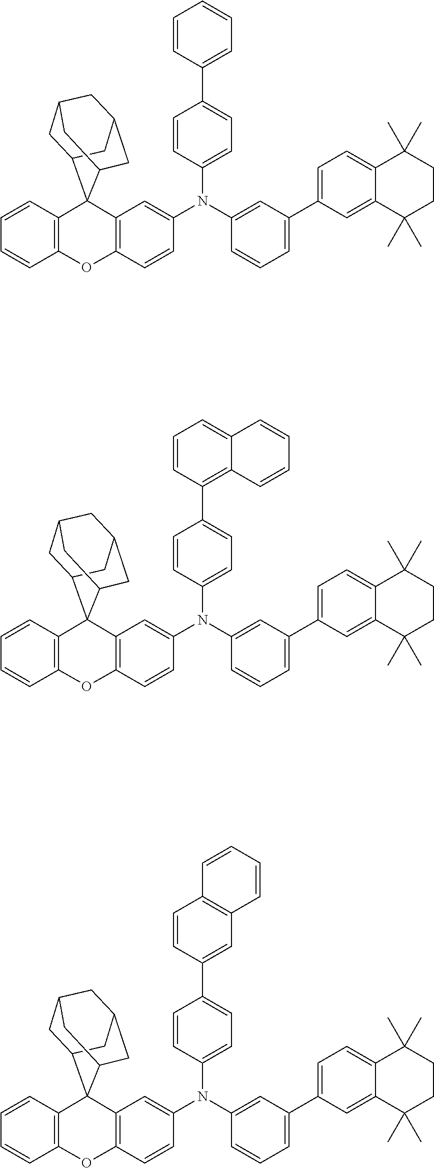 [SMILES: CC1(C)CCC(C)(C)c2cc(-c3cccc(N(c4ccc(-c5ccccc5)cc4)c4ccc5c(c4)C4(c6ccccc6O5)C5CC6CC(C5)CC4C6)c3)ccc21 ; CC1(C)CCC(C)(C)c2cc(-c3cccc(N(c4ccc(-c5cccc6ccccc56)cc4)c4ccc5c(c4)C4(c6ccccc6O5)C5CC6CC(C5)CC4C6)c3)ccc21 ; CC1(C)CCC(C)(C)c2cc(-c3cccc(N(c4ccc(-c5ccc6ccccc6c5)cc4)c4ccc5c(c4)C4(c6ccccc6O5)C5CC6CC(C5)CC4C6)c3)ccc21]

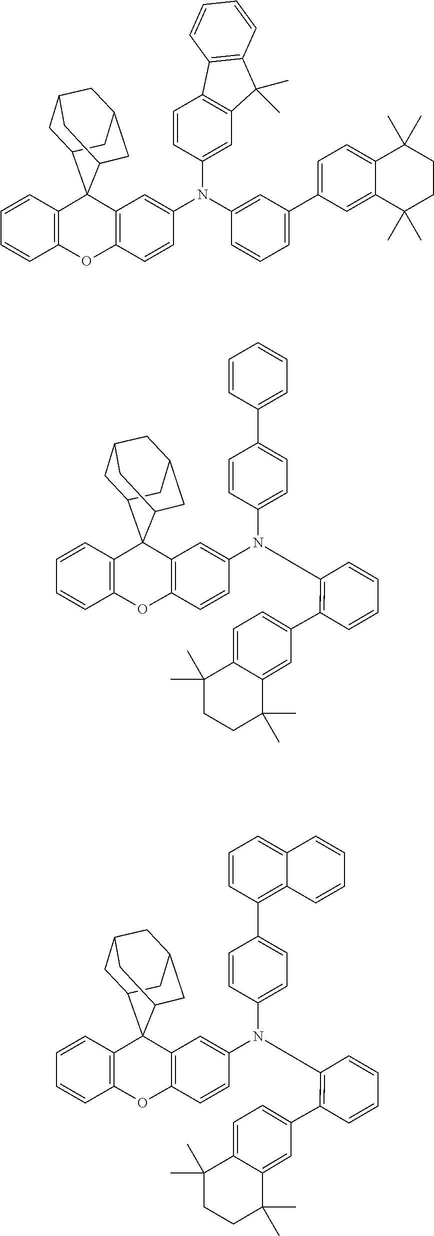 [SMILES: CC1(C)CCC(C)(C)c2cc(-c3cccc(N(c4ccc5c(c4)C(C)(C)c4ccccc4-5)c4ccc5c(c4)C4(c6ccccc6O5)C5CC6CC(C5)CC4C6)c3)ccc21 ; CC1(C)CCC(C)(C)c2cc(-c3ccccc3N(c3ccc(-c4ccccc4)cc3)c3ccc4c(c3)C3(c5ccccc5O4)C4CC5CC(C4)CC3C5)ccc21 ; CC1(C)CCC(C)(C)c2cc(-c3ccccc3N(c3ccc(-c4cccc5ccccc45)cc3)c3ccc4c(c3)C3(c5ccccc5O4)C4CC5CC(C4)CC3C5)ccc21]

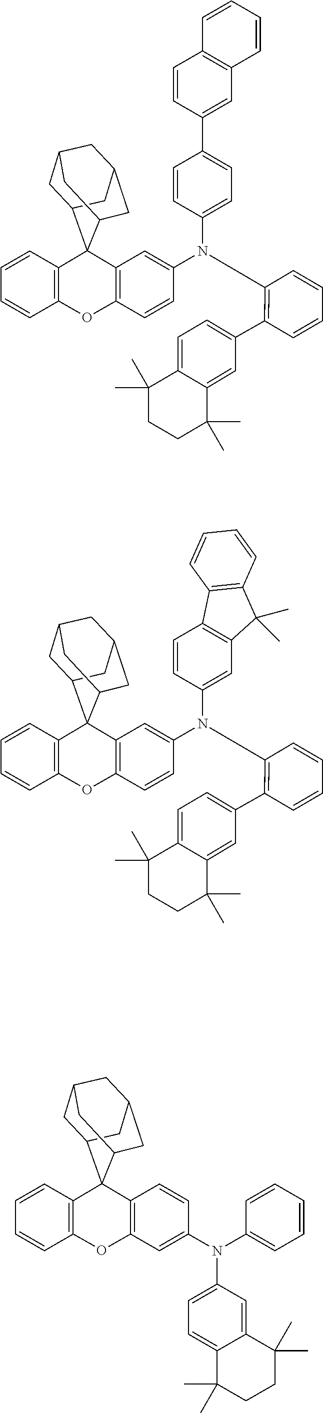 [SMILES: CC1(C)CCC(C)(C)c2cc(-c3ccccc3N(c3ccc(-c4ccc5ccccc5c4)cc3)c3ccc4c(c3)C3(c5ccccc5O4)C4CC5CC(C4)CC3C5)ccc21 ; CC1(C)CCC(C)(C)c2cc(-c3ccccc3N(c3ccc4c(c3)C(C)(C)c3ccccc3-4)c3ccc4c(c3)C3(c5ccccc5O4)C4CC5CC(C4)CC3C5)ccc21 ; CC1(C)CCC(C)(C)c2cc(N(c3ccccc3)c3ccc4c(c3)Oc3ccccc3C43C4CC5CC(C4)CC3C5)ccc21]

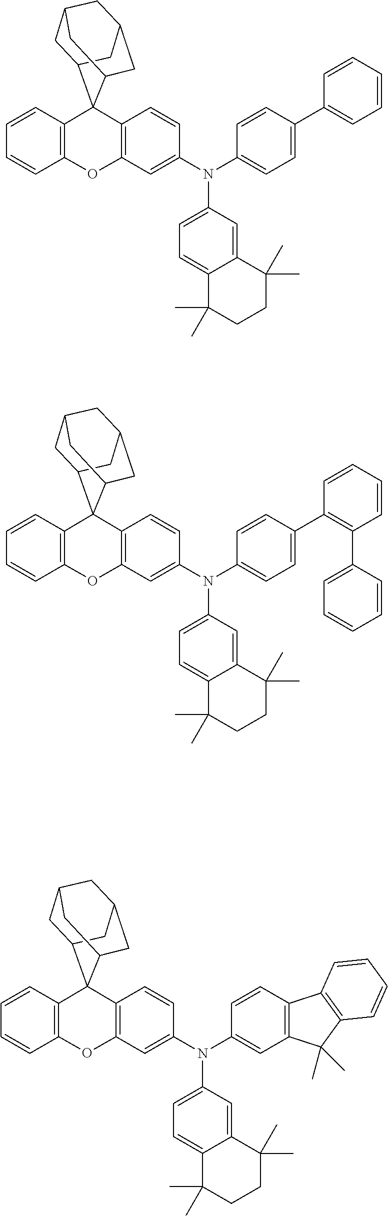 [SMILES: CC1(C)CCC(C)(C)c2cc(N(c3ccc(-c4ccccc4)cc3)c3ccc4c(c3)Oc3ccccc3C43C4CC5CC(C4)CC3C5)ccc21 ; CC1(C)CCC(C)(C)c2cc(N(c3ccc(-c4ccccc4-c4ccccc4)cc3)c3ccc4c(c3)Oc3ccccc3C43C4CC5CC(C4)CC3C5)ccc21 ; CC1(C)CCC(C)(C)c2cc(N(c3ccc4c(c3)Oc3ccccc3C43C4CC5CC(C4)CC3C5)c3ccc4c(c3)C(C)(C)c3ccccc3-4)ccc21]

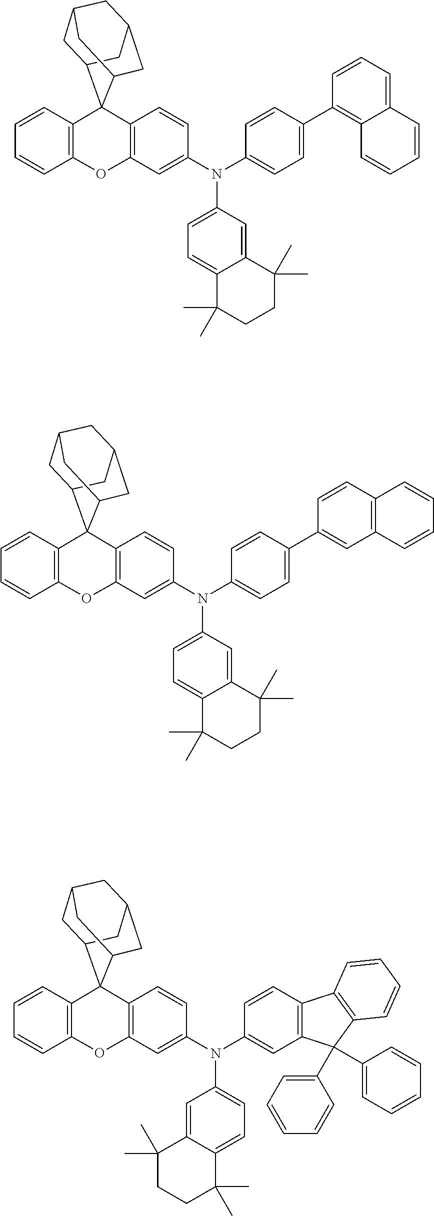 [SMILES: CC1(C)CCC(C)(C)c2cc(N(c3ccc(-c4cccc5ccccc45)cc3)c3ccc4c(c3)Oc3ccccc3C43C4CC5CC(C4)CC3C5)ccc21 ; CC1(C)CCC(C)(C)c2cc(N(c3ccc(-c4ccc5ccccc5c4)cc3)c3ccc4c(c3)Oc3ccccc3C43C4CC5CC(C4)CC3C5)ccc21 ; CC1(C)CCC(C)(C)c2cc(N(c3ccc4c(c3)Oc3ccccc3C43C4CC5CC(C4)CC3C5)c3ccc4c(c3)C(c3ccccc3)(c3ccccc3)c3ccccc3-4)ccc21]

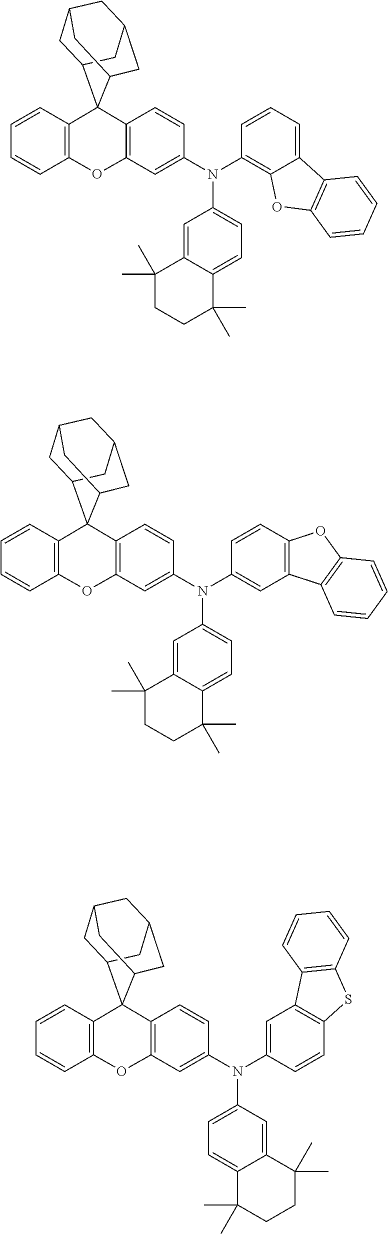 [SMILES: CC1(C)CCC(C)(C)c2cc(N(c3ccc4c(c3)Oc3ccccc3C43C4CC5CC(C4)CC3C5)c3cccc4c3oc3ccccc34)ccc21 ; CC1(C)CCC(C)(C)c2cc(N(c3ccc4c(c3)Oc3ccccc3C43C4CC5CC(C4)CC3C5)c3ccc4oc5ccccc5c4c3)ccc21 ; CC1(C)CCC(C)(C)c2cc(N(c3ccc4c(c3)Oc3ccccc3C43C4CC5CC(C4)CC3C5)c3ccc4sc5ccccc5c4c3)ccc21]

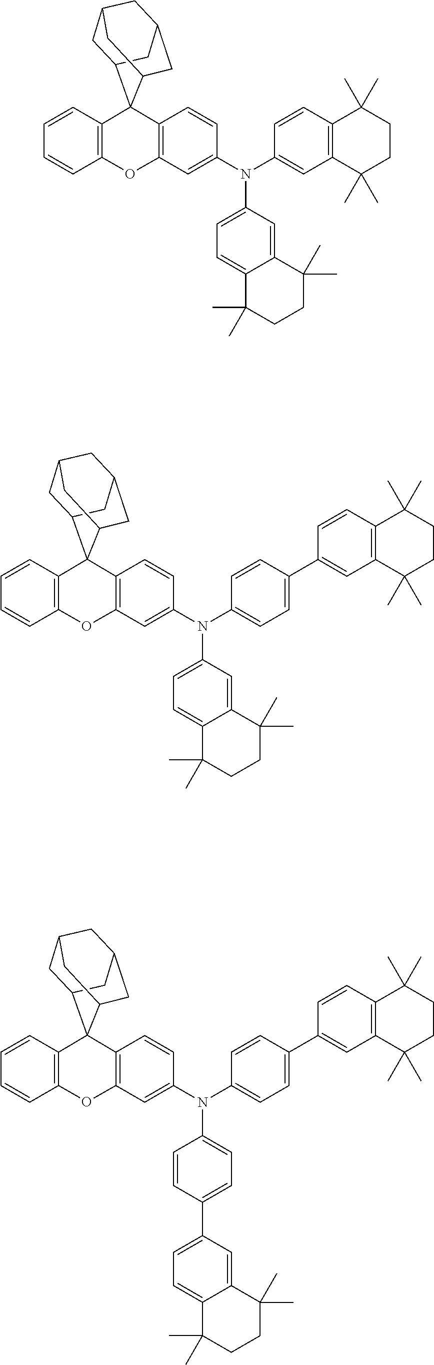 [SMILES: CC1(C)CCC(C)(C)c2cc(N(c3ccc4c(c3)Oc3ccccc3C43C4CC5CC(C4)CC3C5)c3ccc4c(c3)C(C)(C)CCC4(C)C)ccc21 ; CC1(C)CCC(C)(C)c2cc(-c3ccc(N(c4ccc5c(c4)Oc4ccccc4C54C5CC6CC(C5)CC4C6)c4ccc5c(c4)C(C)(C)CCC5(C)C)cc3)ccc21 ; CC1(C)CCC(C)(C)c2cc(-c3ccc(N(c4ccc(-c5ccc6c(c5)C(C)(C)CCC6(C)C)cc4)c4ccc5c(c4)Oc4ccccc4C54C5CC6CC(C5)CC4C6)cc3)ccc21]

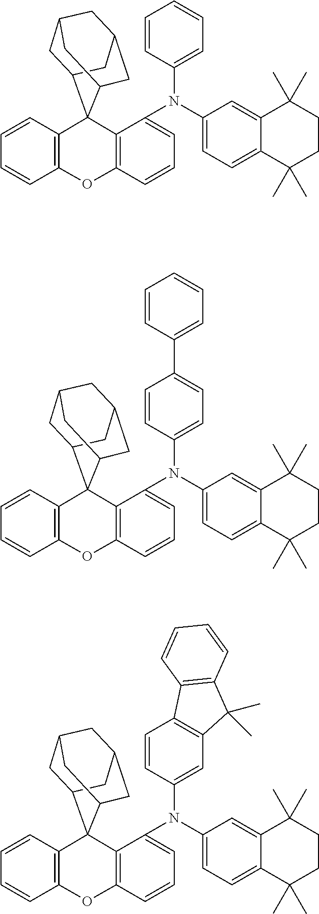 [SMILES: CC1(C)CCC(C)(C)c2cc(N(c3ccccc3)c3cccc4c3C3(c5ccccc5O4)C4CC5CC(C4)CC3C5)ccc21 ; CC1(C)CCC(C)(C)c2cc(N(c3ccc(-c4ccccc4)cc3)c3cccc4c3C3(c5ccccc5O4)C4CC5CC(C4)CC3C5)ccc21 ; CC1(C)CCC(C)(C)c2cc(N(c3ccc4c(c3)C(C)(C)c3ccccc3-4)c3cccc4c3C3(c5ccccc5O4)C4CC5CC(C4)CC3C5)ccc21]

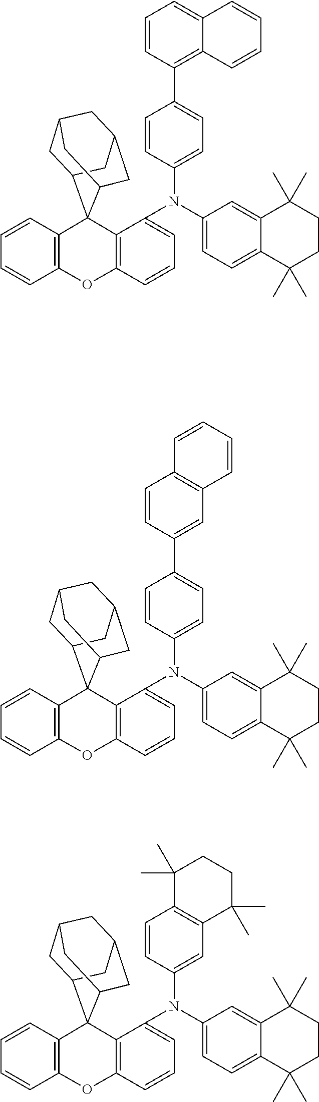 [SMILES: CC1(C)CCC(C)(C)c2cc(N(c3ccc(-c4cccc5ccccc45)cc3)c3cccc4c3C3(c5ccccc5O4)C4CC5CC(C4)CC3C5)ccc21 ; CC1(C)CCC(C)(C)c2cc(N(c3ccc(-c4ccc5ccccc5c4)cc3)c3cccc4c3C3(c5ccccc5O4)C4CC5CC(C4)CC3C5)ccc21 ; CC1(C)CCC(C)(C)c2cc(N(c3ccc4c(c3)C(C)(C)CCC4(C)C)c3cccc4c3C3(c5ccccc5O4)C4CC5CC(C4)CC3C5)ccc21]

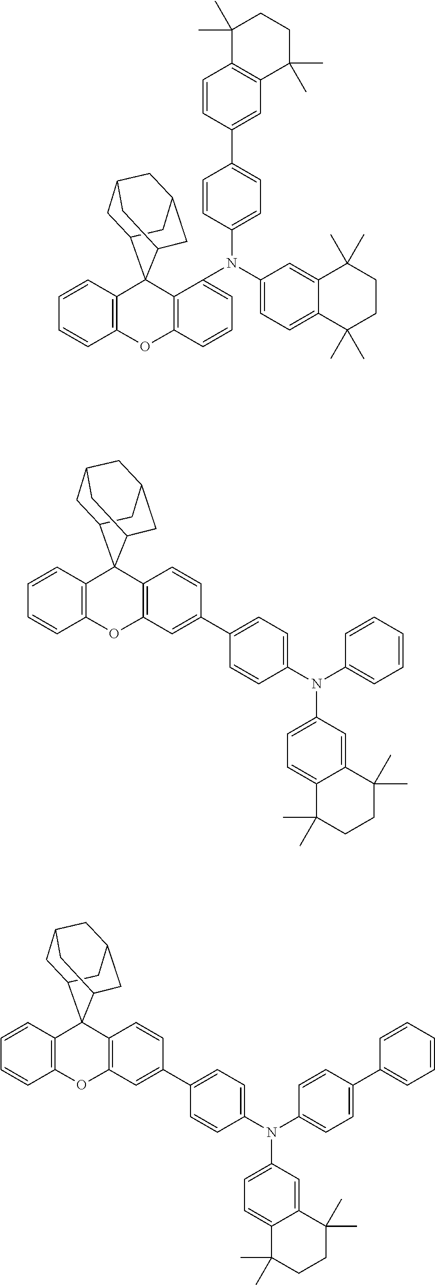 [SMILES: CC1(C)CCC(C)(C)c2cc(-c3ccc(N(c4ccc5c(c4)C(C)(C)CCC5(C)C)c4cccc5c4C4(c6ccccc6O5)C5CC6CC(C5)CC4C6)cc3)ccc21 ; CC1(C)CCC(C)(C)c2cc(N(c3ccccc3)c3ccc(-c4ccc5c(c4)Oc4ccccc4C54C5CC6CC(C5)CC4C6)cc3)ccc21 ; CC1(C)CCC(C)(C)c2cc(N(c3ccc(-c4ccccc4)cc3)c3ccc(-c4ccc5c(c4)Oc4ccccc4C54C5CC6CC(C5)CC4C6)cc3)ccc21]

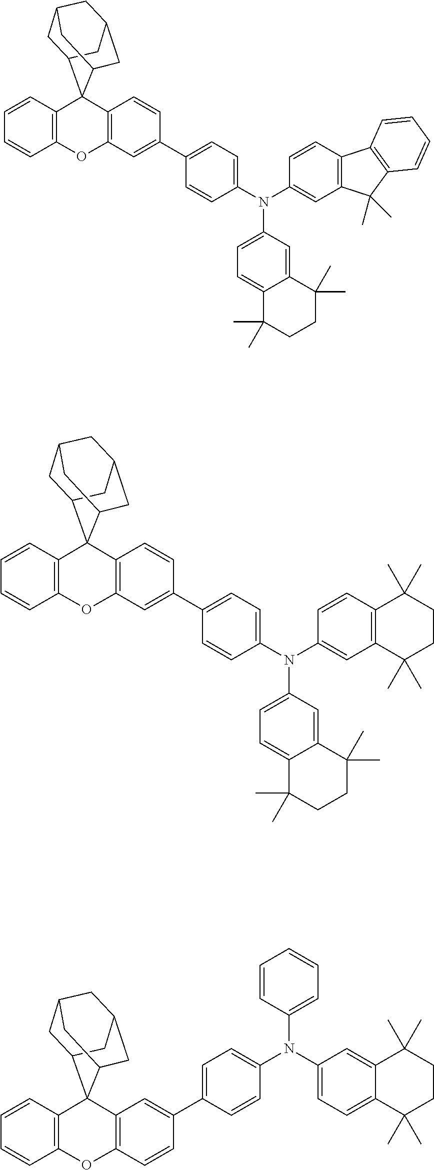 [SMILES: CC1(C)CCC(C)(C)c2cc(N(c3ccc(-c4ccc5c(c4)Oc4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc5c(c4)Oc4ccccc4C54C5CC6CC(C5)CC4C6)cc3)c3ccc4c(c3)C(C)(C)CCC4(C)C)ccc21 ; CC1(C)CCC(C)(C)c2cc(N(c3ccccc3)c3ccc(-c4ccc5c(c4)C4(c6ccccc6O5)C5CC6CC(C5)CC4C6)cc3)ccc21]

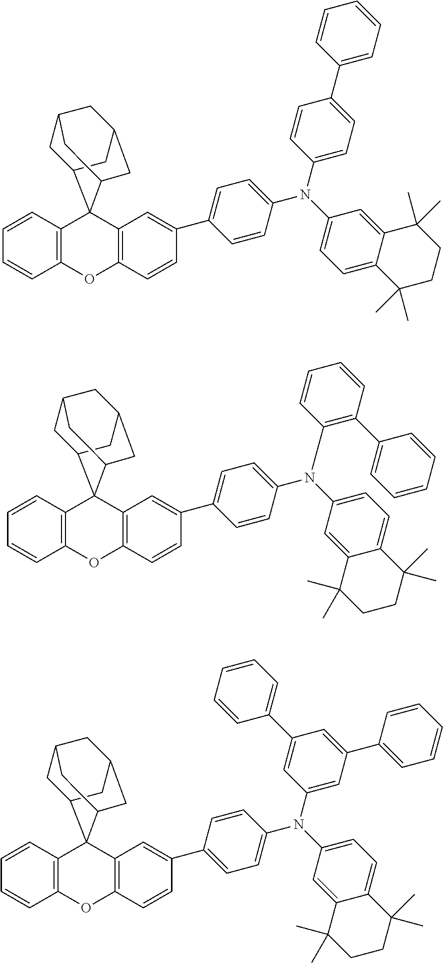 [SMILES: CC1(C)CCC(C)(C)c2cc(N(c3ccc(-c4ccccc4)cc3)c3ccc(-c4ccc5c(c4)C4(c6ccccc6O5)C5CC6CC(C5)CC4C6)cc3)ccc21 ; CC1(C)CCC(C)(C)c2cc(N(c3ccc(-c4ccc5c(c4)C4(c6ccccc6O5)C5CC6CC(C5)CC4C6)cc3)c3ccccc3-c3ccccc3)ccc21 ; CC1(C)CCC(C)(C)c2cc(N(c3ccc(-c4ccc5c(c4)C4(c6ccccc6O5)C5CC6CC(C5)CC4C6)cc3)c3cc(-c4ccccc4)cc(-c4ccccc4)c3)ccc21]

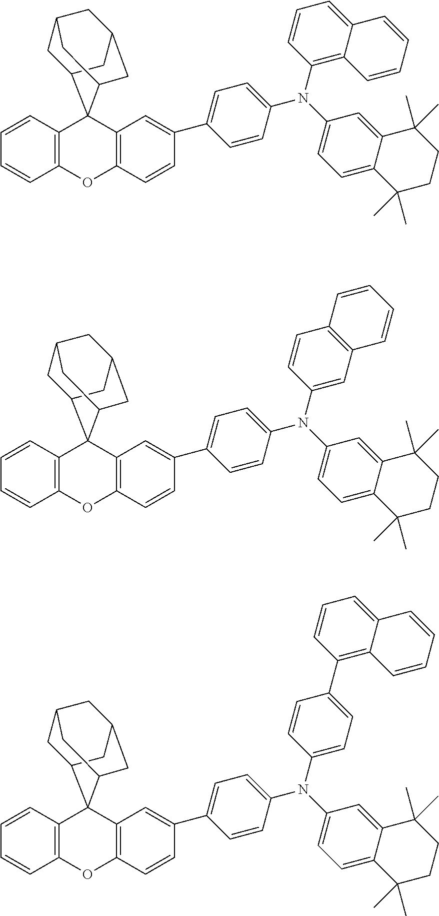 [SMILES: CC1(C)CCC(C)(C)c2cc(N(c3ccc(-c4ccc5c(c4)C4(c6ccccc6O5)C5CC6CC(C5)CC4C6)cc3)c3cccc4ccccc34)ccc21 ; CC1(C)CCC(C)(C)c2cc(N(c3ccc(-c4ccc5c(c4)C4(c6ccccc6O5)C5CC6CC(C5)CC4C6)cc3)c3ccc4ccccc4c3)ccc21 ; CC1(C)CCC(C)(C)c2cc(N(c3ccc(-c4ccc5c(c4)C4(c6ccccc6O5)C5CC6CC(C5)CC4C6)cc3)c3ccc(-c4cccc5ccccc45)cc3)ccc21]

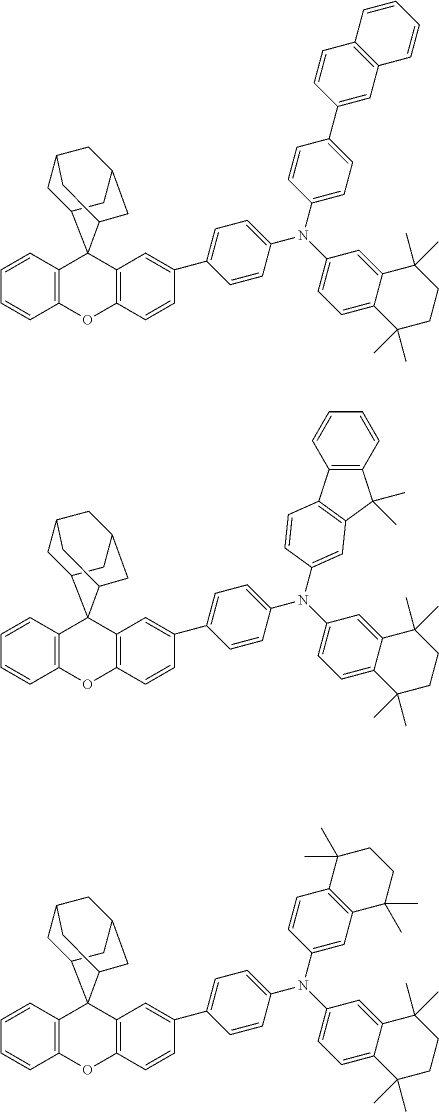 [SMILES: CC1(C)CCC(C)(C)c2cc(N(c3ccc(-c4ccc5c(c4)C4(c6ccccc6O5)C5CC6CC(C5)CC4C6)cc3)c3ccc(-c4ccc5ccccc5c4)cc3)ccc21 ; CC1(C)CCC(C)(C)c2cc(N(c3ccc(-c4ccc5c(c4)C4(c6ccccc6O5)C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc5c(c4)C4(c6ccccc6O5)C5CC6CC(C5)CC4C6)cc3)c3ccc4c(c3)C(C)(C)CCC4(C)C)ccc21]

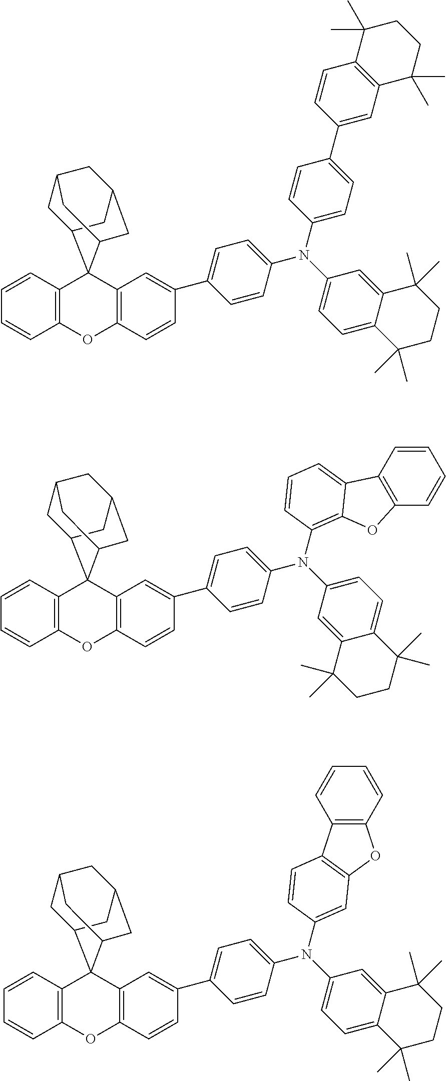 [SMILES: CC1(C)CCC(C)(C)c2cc(-c3ccc(N(c4ccc(-c5ccc6c(c5)C5(c7ccccc7O6)C6CC7CC(C6)CC5C7)cc4)c4ccc5c(c4)C(C)(C)CCC5(C)C)cc3)ccc21 ; CC1(C)CCC(C)(C)c2cc(N(c3ccc(-c4ccc5c(c4)C4(c6ccccc6O5)C5CC6CC(C5)CC4C6)cc3)c3cccc4c3oc3ccccc34)ccc21 ; CC1(C)CCC(C)(C)c2cc(N(c3ccc(-c4ccc5c(c4)C4(c6ccccc6O5)C5CC6CC(C5)CC4C6)cc3)c3ccc4c(c3)oc3ccccc34)ccc21]

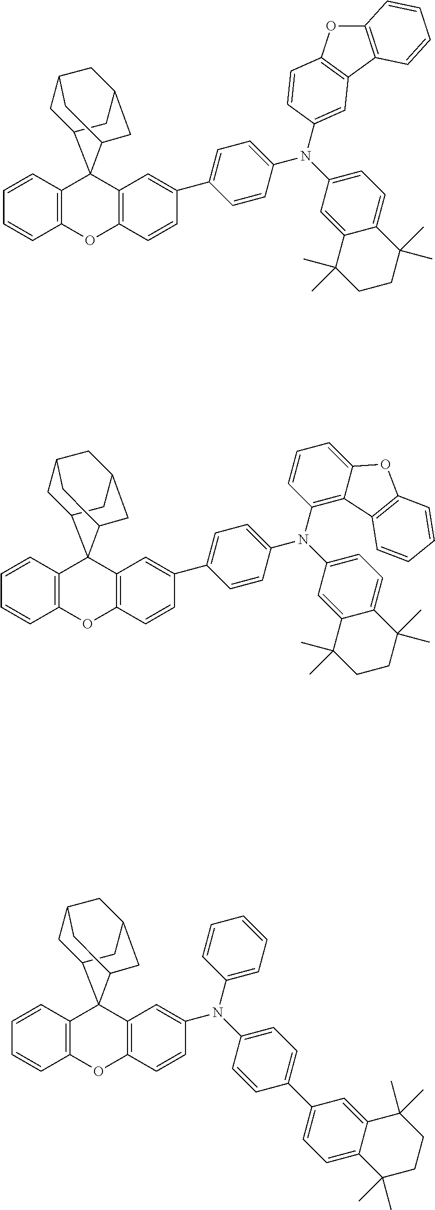 [SMILES: CC1(C)CCC(C)(C)c2cc(N(c3ccc(-c4ccc5c(c4)C4(c6ccccc6O5)C5CC6CC(C5)CC4C6)cc3)c3ccc4oc5ccccc5c4c3)ccc21 ; CC1(C)CCC(C)(C)c2cc(N(c3ccc(-c4ccc5c(c4)C4(c6ccccc6O5)C5CC6CC(C5)CC4C6)cc3)c3cccc4oc5ccccc5c34)ccc21 ; CC1(C)CCC(C)(C)c2cc(-c3ccc(N(c4ccccc4)c4ccc5c(c4)C4(c6ccccc6O5)C5CC6CC(C5)CC4C6)cc3)ccc21]

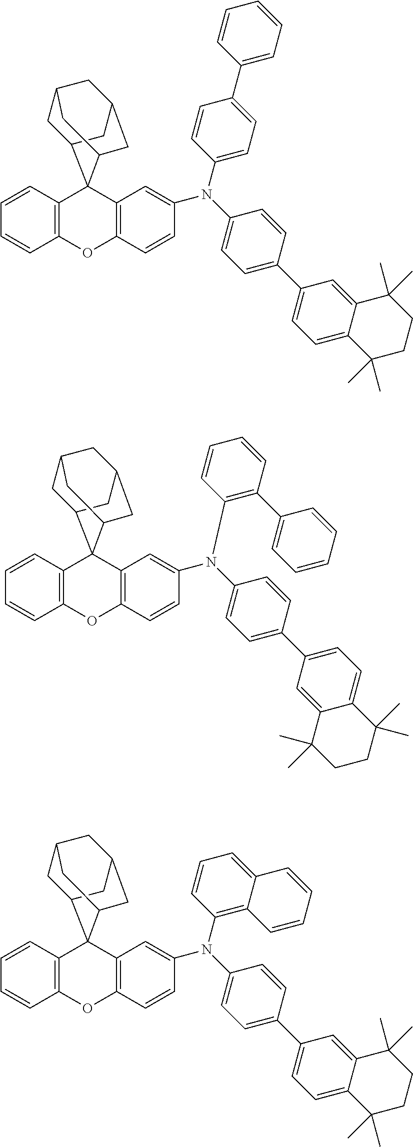 [SMILES: CC1(C)CCC(C)(C)c2cc(-c3ccc(N(c4ccc(-c5ccccc5)cc4)c4ccc5c(c4)C4(c6ccccc6O5)C5CC6CC(C5)CC4C6)cc3)ccc21 ; CC1(C)CCC(C)(C)c2cc(-c3ccc(N(c4ccc5c(c4)C4(c6ccccc6O5)C5CC6CC(C5)CC4C6)c4ccccc4-c4ccccc4)cc3)ccc21 ; CC1(C)CCC(C)(C)c2cc(-c3ccc(N(c4ccc5c(c4)C4(c6ccccc6O5)C5CC6CC(C5)CC4C6)c4cccc5ccccc45)cc3)ccc21]

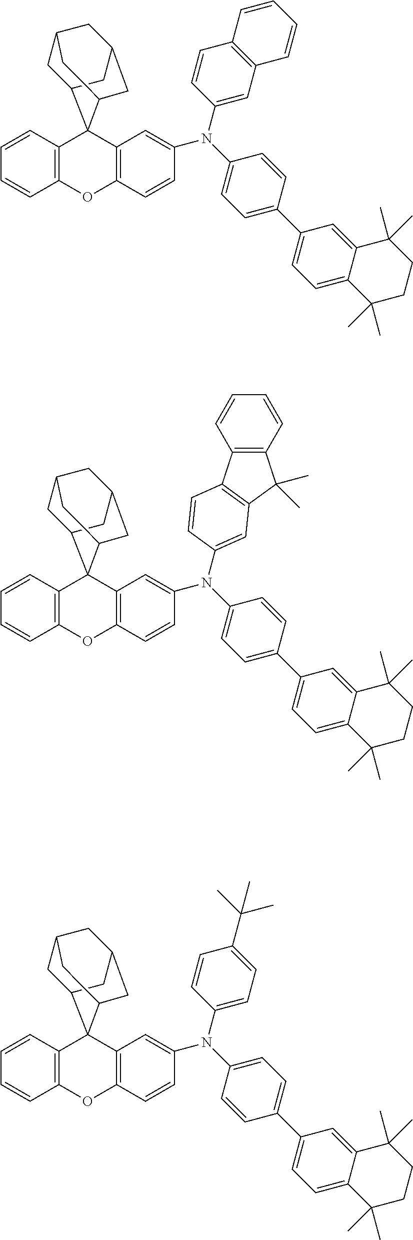 [SMILES: CC1(C)CCC(C)(C)c2cc(-c3ccc(N(c4ccc5c(c4)C4(c6ccccc6O5)C5CC6CC(C5)CC4C6)c4ccc5ccccc5c4)cc3)ccc21 ; CC1(C)CCC(C)(C)c2cc(-c3ccc(N(c4ccc5c(c4)C(C)(C)c4ccccc4-5)c4ccc5c(c4)C4(c6ccccc6O5)C5CC6CC(C5)CC4C6)cc3)ccc21 ; CC(C)(C)c1ccc(N(c2ccc(-c3ccc4c(c3)C(C)(C)CCC4(C)C)cc2)c2ccc3c(c2)C2(c4ccccc4O3)C3CC4CC(C3)CC2C4)cc1]

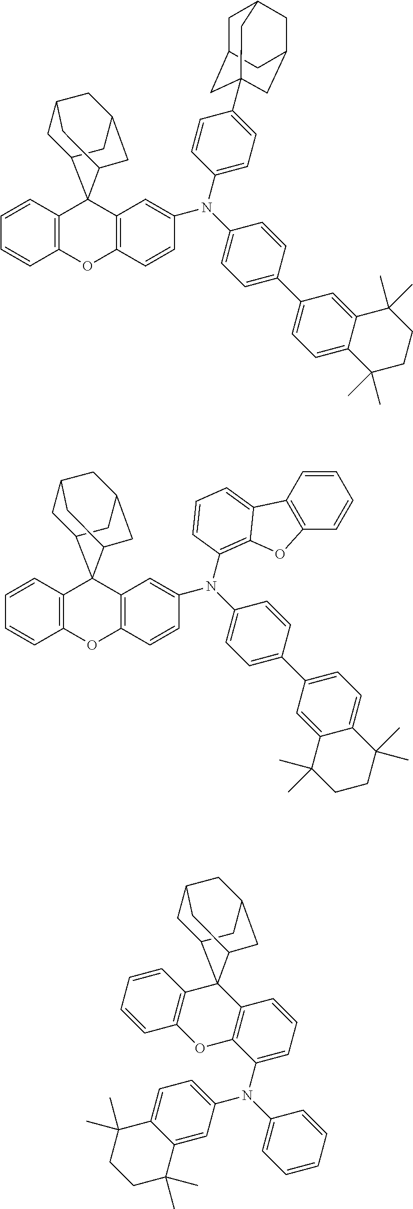

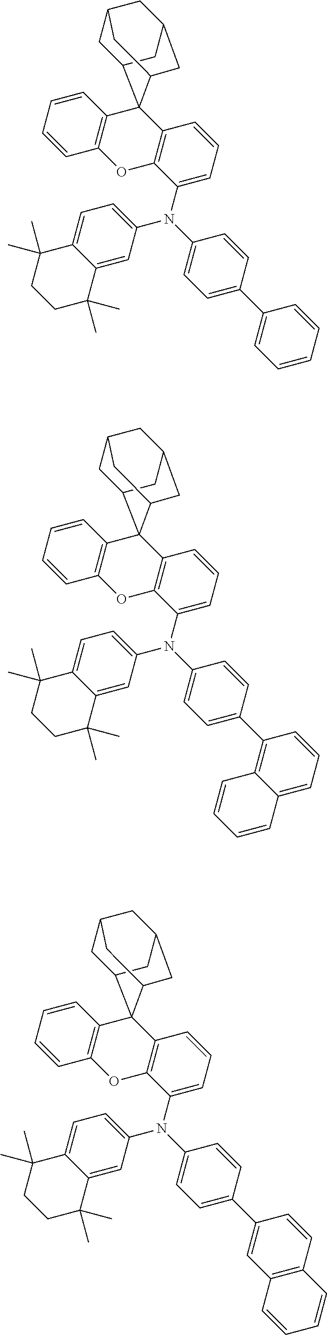 [SMILES: CC1(C)CCC(C)(C)c2cc(N(c3ccc(-c4ccccc4)cc3)c3cccc4c3Oc3ccccc3C43C4CC5CC(C4)CC3C5)ccc21 ; CC1(C)CCC(C)(C)c2cc(N(c3ccc(-c4cccc5ccccc45)cc3)c3cccc4c3Oc3ccccc3C43C4CC5CC(C4)CC3C5)ccc21 ; CC1(C)CCC(C)(C)c2cc(N(c3ccc(-c4ccc5ccccc5c4)cc3)c3cccc4c3Oc3ccccc3C43C4CC5CC(C4)CC3C5)ccc21]

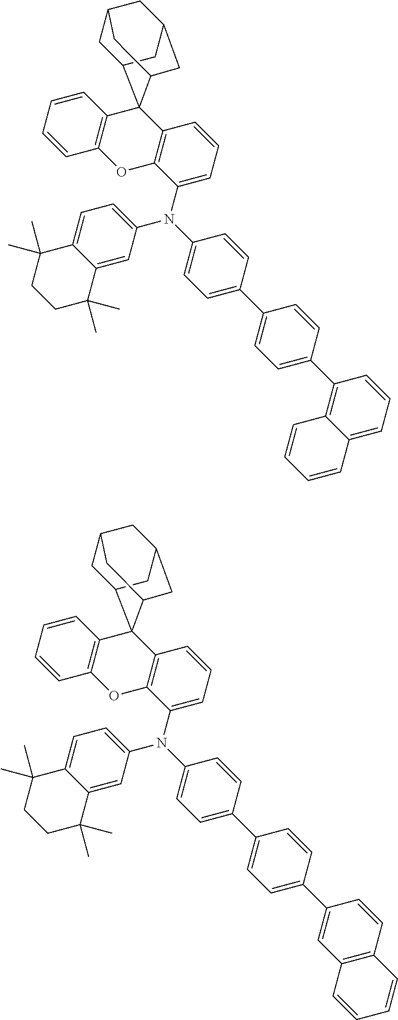 [SMILES: CC1(C)CCC(C)(C)c2cc(N(c3ccc(-c4ccc(-c5cccc6ccccc56)cc4)cc3)c3cccc4c3Oc3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3cccc4c3Oc3ccccc3C43C4CC5CC(C4)CC3C5)ccc21]

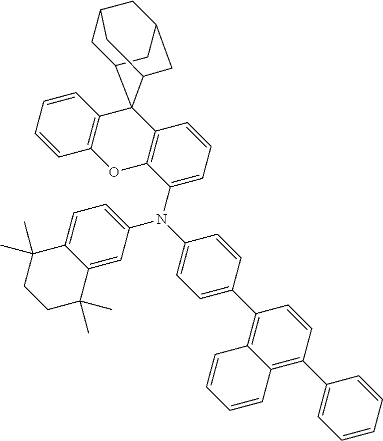 [SMILES: CC1(C)CCC(C)(C)c2cc(N(c3ccc(-c4ccc(-c5ccccc5)c5ccccc45)cc3)c3cccc4c3Oc3ccccc3C43C4CC5CC(C4)CC3C5)ccc21]

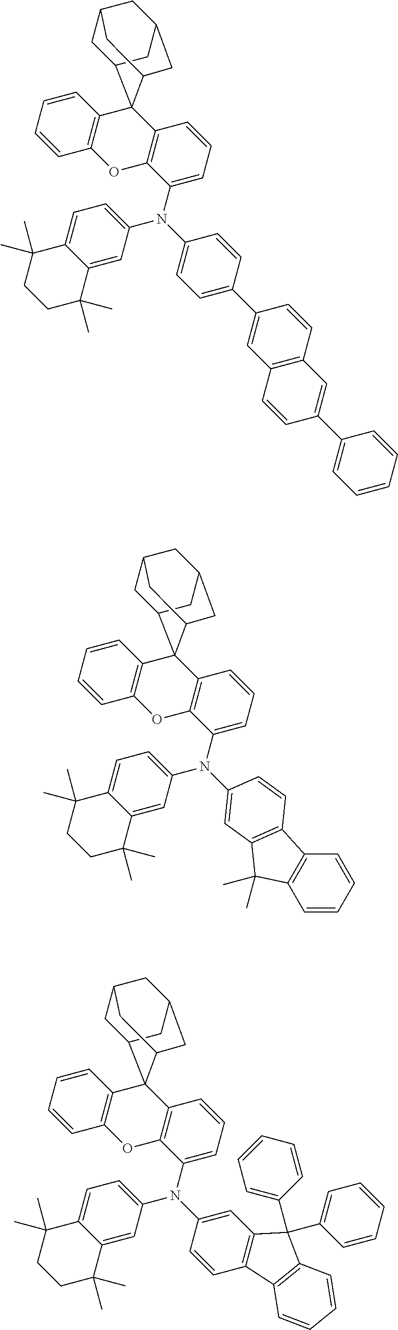 [SMILES: CC1(C)CCC(C)(C)c2cc(N(c3ccc(-c4ccc5cc(-c6ccccc6)ccc5c4)cc3)c3cccc4c3Oc3ccccc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3ccccc3C43C4CC5CC(C4)CC3C5)ccc21 ; CC1(C)CCC(C)(C)c2cc(N(c3ccc4c(c3)C(c3ccccc3)(c3ccccc3)c3ccccc3-4)c3cccc4c3Oc3ccccc3C43C4CC5CC(C4)CC3C5)ccc21]

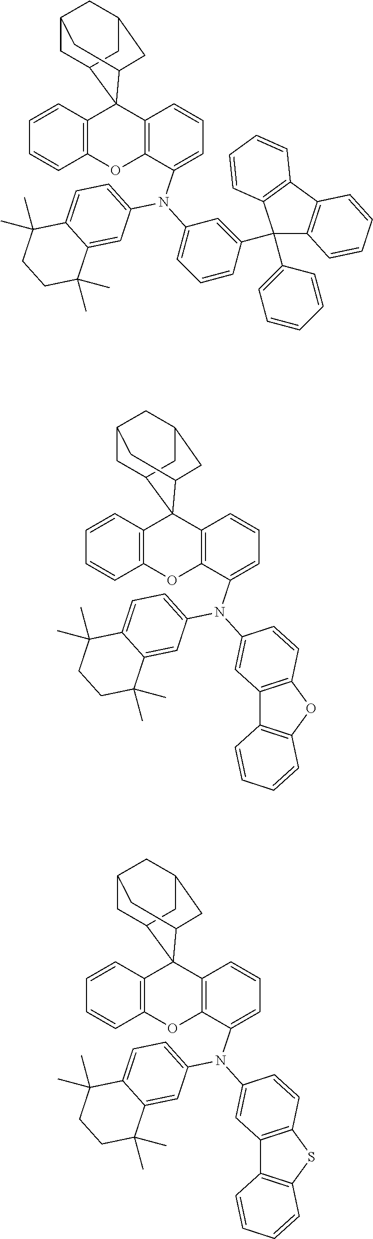 [SMILES: CC1(C)CCC(C)(C)c2cc(N(c3cccc(C4(c5ccccc5)c5ccccc5-c5ccccc54)c3)c3cccc4c3Oc3ccccc3C43C4CC5CC(C4)CC3C5)ccc21 ; CC1(C)CCC(C)(C)c2cc(N(c3ccc4oc5ccccc5c4c3)c3cccc4c3Oc3ccccc3C43C4CC5CC(C4)CC3C5)ccc21 ; CC1(C)CCC(C)(C)c2cc(N(c3ccc4sc5ccccc5c4c3)c3cccc4c3Oc3ccccc3C43C4CC5CC(C4)CC3C5)ccc21]

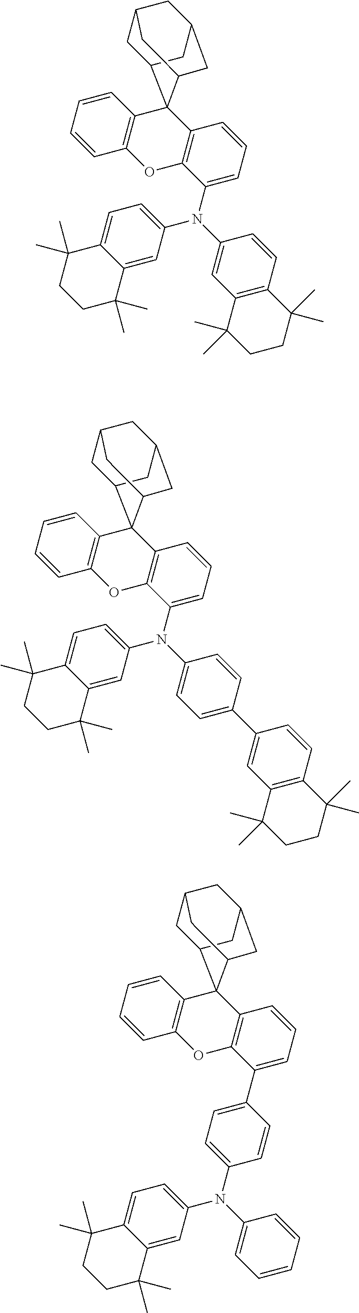

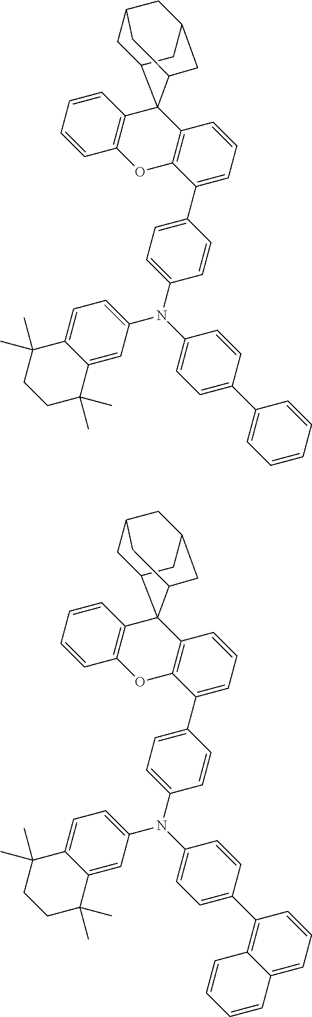 [SMILES: CC1(C)CCC(C)(C)c2cc(N(c3ccc(-c4ccccc4)cc3)c3ccc(-c4cccc5c4Oc4ccccc4C54C5CC6CC(C5)CC4C6)cc3)ccc21 ; CC1(C)CCC(C)(C)c2cc(N(c3ccc(-c4cccc5c4Oc4ccccc4C54C5CC6CC(C5)CC4C6)cc3)c3ccc(-c4cccc5ccccc45)cc3)ccc21]

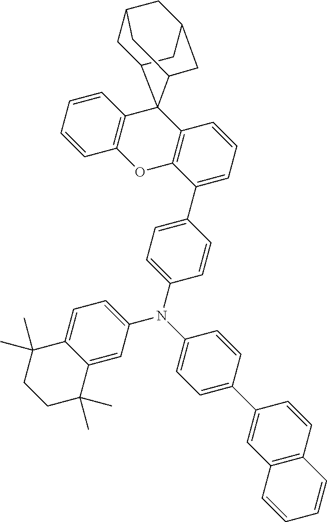 [SMILES: CC1(C)CCC(C)(C)c2cc(N(c3ccc(-c4ccc5ccccc5c4)cc3)c3ccc(-c4cccc5c4Oc4ccccc4C54C5CC6CC(C5)CC4C6)cc3)ccc21]

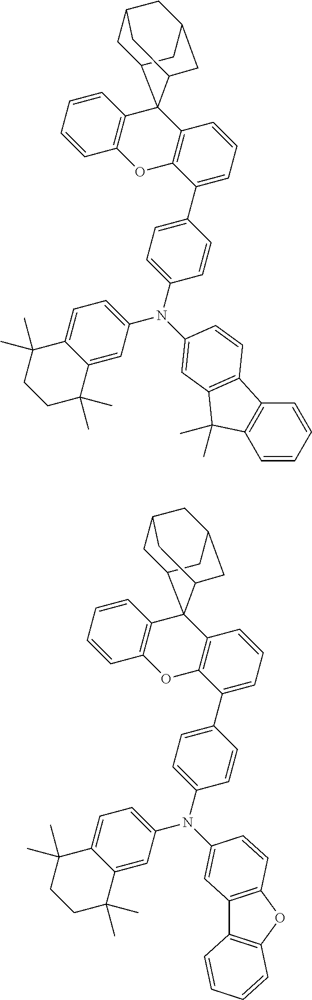 [SMILES: CC1(C)CCC(C)(C)c2cc(N(c3ccc(-c4cccc5c4Oc4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4cccc5c4Oc4ccccc4C54C5CC6CC(C5)CC4C6)cc3)c3ccc4oc5ccccc5c4c3)ccc21]

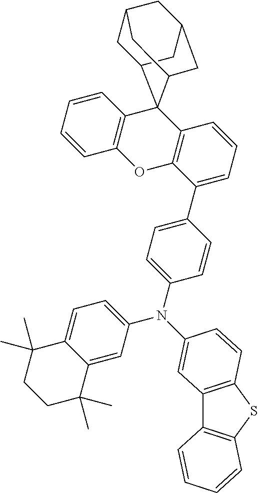 [SMILES: CC1(C)CCC(C)(C)c2cc(N(c3ccc(-c4cccc5c4Oc4ccccc4C54C5CC6CC(C5)CC4C6)cc3)c3ccc4sc5ccccc5c4c3)ccc21]

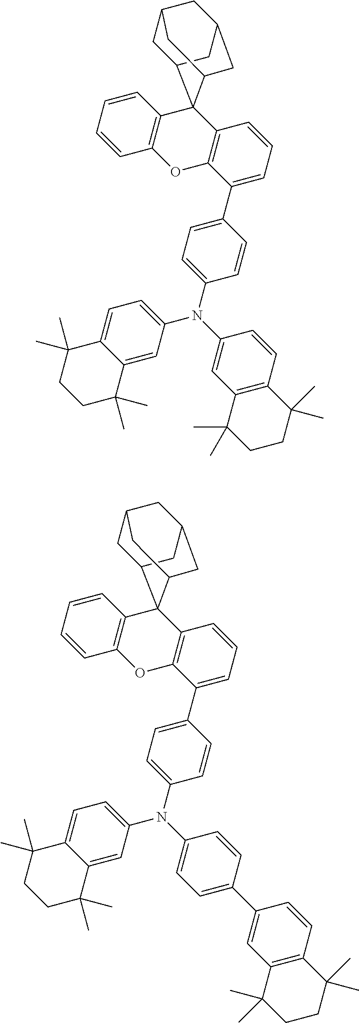

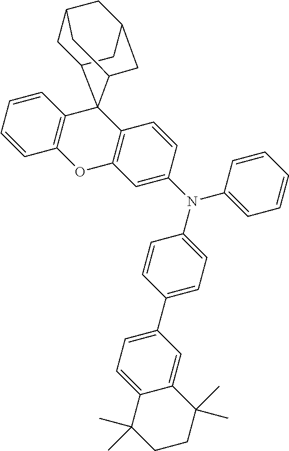 [SMILES: CC1(C)CCC(C)(C)c2cc(-c3ccc(N(c4ccccc4)c4ccc5c(c4)Oc4ccccc4C54C5CC6CC(C5)CC4C6)cc3)ccc21]

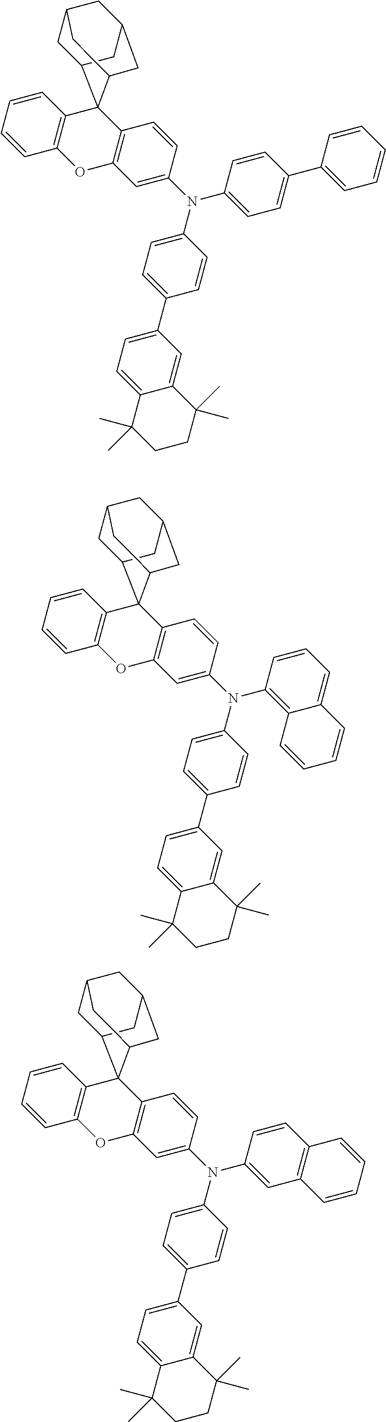

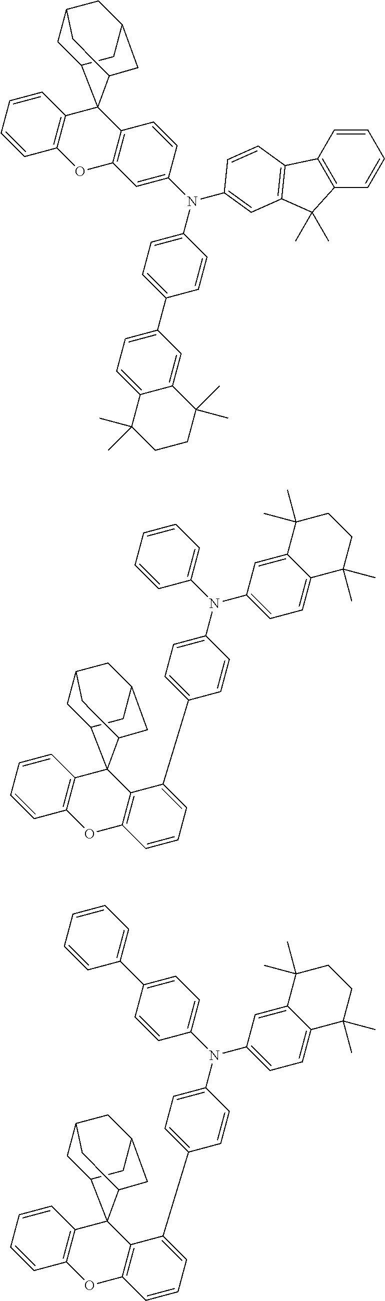 [SMILES: CC1(C)CCC(C)(C)c2cc(-c3ccc(N(c4ccc5c(c4)Oc4ccccc4C54C5CC6CC(C5)CC4C6)c4ccc5c(c4)C(C)(C)c4ccccc4-5)cc3)ccc21 ; CC1(C)CCC(C)(C)c2cc(N(c3ccccc3)c3ccc(-c4cccc5c4C4(c6ccccc6O5)C5CC6CC(C5)CC4C6)cc3)ccc21 ; CC1(C)CCC(C)(C)c2cc(N(c3ccc(-c4ccccc4)cc3)c3ccc(-c4cccc5c4C4(c6ccccc6O5)C5CC6CC(C5)CC4C6)cc3)ccc21]

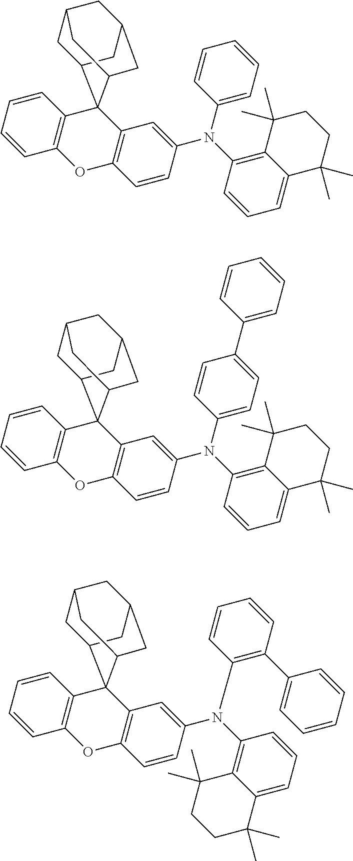

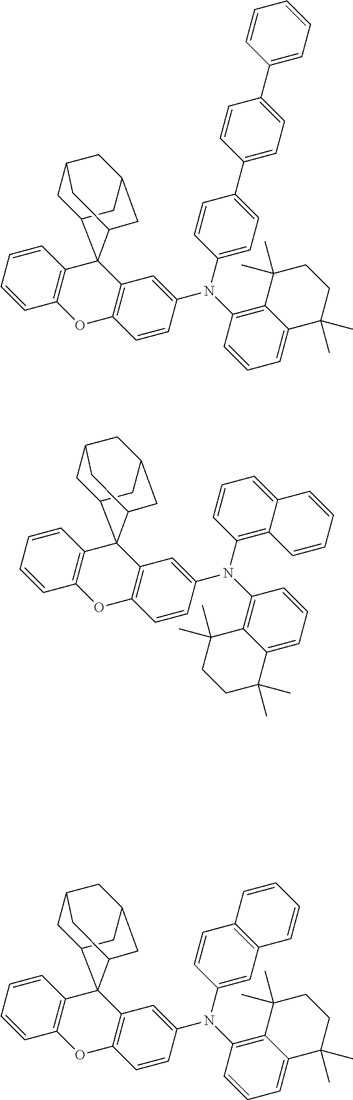 [SMILES: CC1(C)CCC(C)(C)c2c(N(c3ccc(-c4ccc(-c5ccccc5)cc4)cc3)c3ccc4c(c3)C3(c5ccccc5O4)C4CC5CC(C4)CC3C5)cccc21 ; CC1(C)CCC(C)(C)c2c(N(c3ccc4c(c3)C3(c5ccccc5O4)C4CC5CC(C4)CC3C5)c3cccc4ccccc34)cccc21 ; CC1(C)CCC(C)(C)c2c(N(c3ccc4c(c3)C3(c5ccccc5O4)C4CC5CC(C4)CC3C5)c3ccc4ccccc4c3)cccc21]

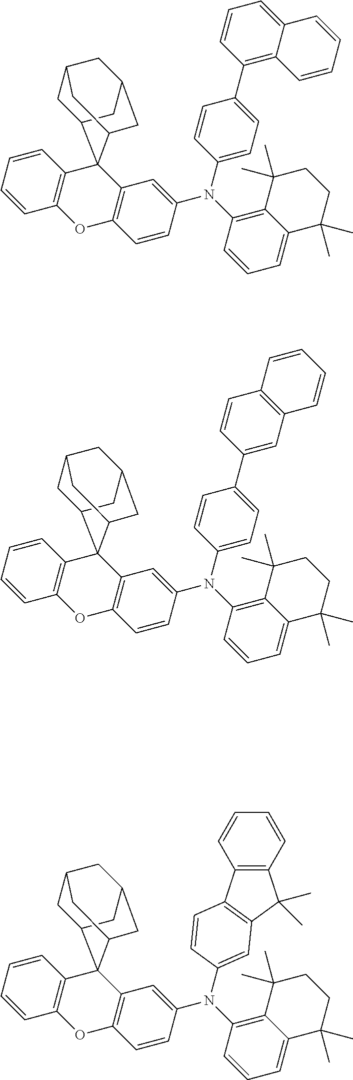 [SMILES: CC1(C)CCC(C)(C)c2c(N(c3ccc(-c4cccc5ccccc45)cc3)c3ccc4c(c3)C3(c5ccccc5O4)C4CC5CC(C4)CC3C5)cccc21 ; CC1(C)CCC(C)(C)c2c(N(c3ccc(-c4ccc5ccccc5c4)cc3)c3ccc4c(c3)C3(c5ccccc5O4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5ccccc5O4)C4CC5CC(C4)CC3C5)cccc21]

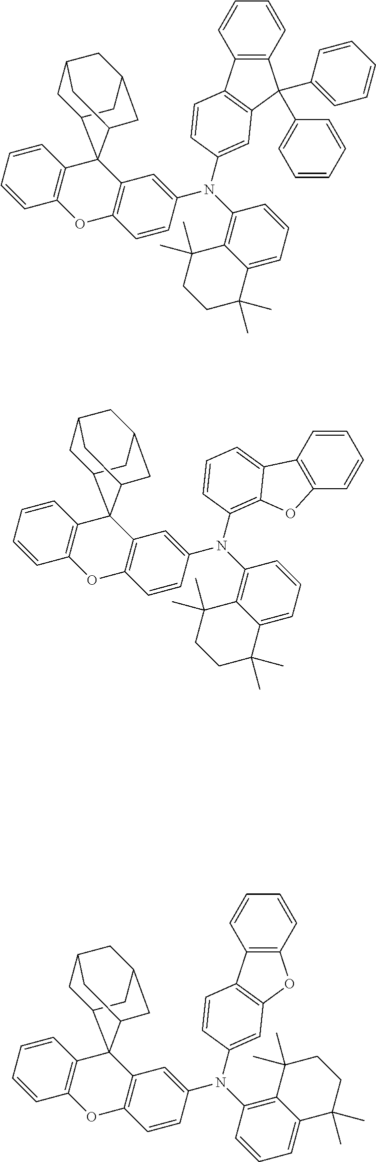 [SMILES: CC1(C)CCC(C)(C)c2c(N(c3ccc4c(c3)C(c3ccccc3)(c3ccccc3)c3ccccc3-4)c3ccc4c(c3)C3(c5ccccc5O4)C4CC5CC(C4)CC3C5)cccc21 ; CC1(C)CCC(C)(C)c2c(N(c3ccc4c(c3)C3(c5ccccc5O4)C4CC5CC(C4)CC3C5)c3cccc4c3oc3ccccc34)cccc21 ; CC1(C)CCC(C)(C)c2c(N(c3ccc4c(c3)C3(c5ccccc5O4)C4CC5CC(C4)CC3C5)c3ccc4c(c3)oc3ccccc34)cccc21]

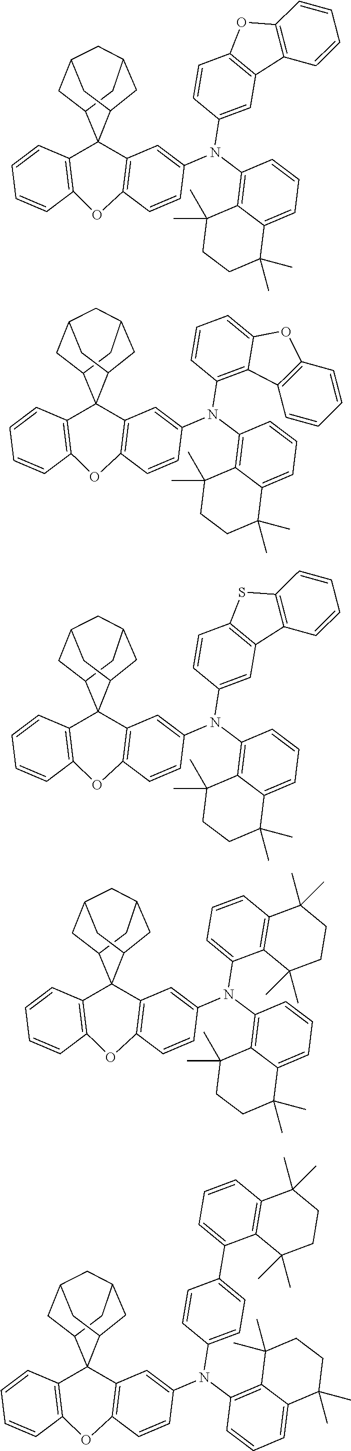

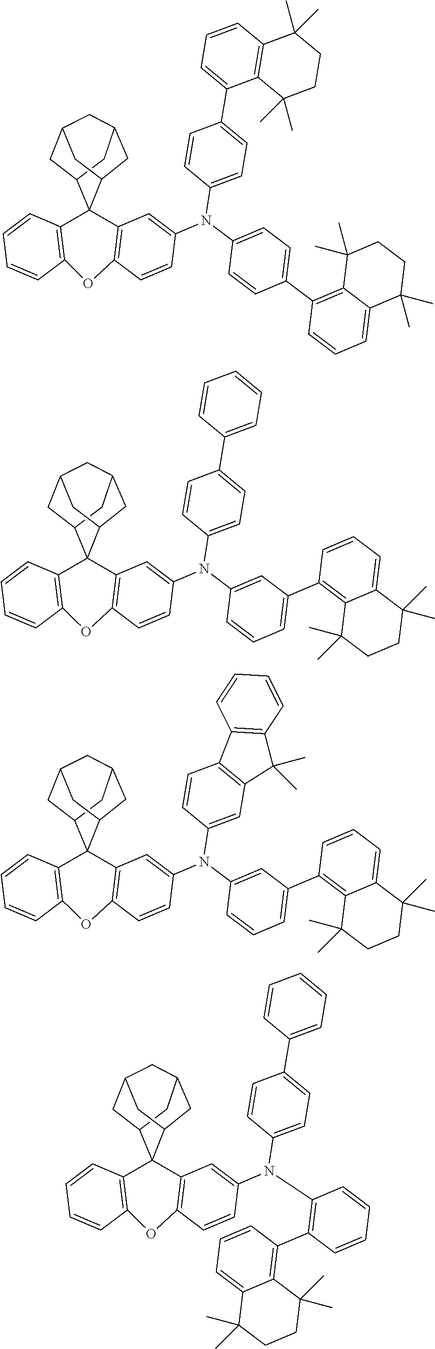

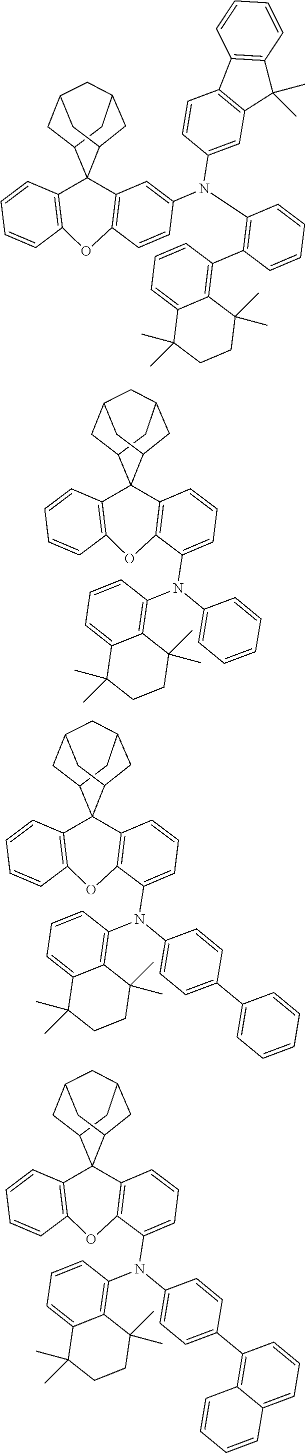

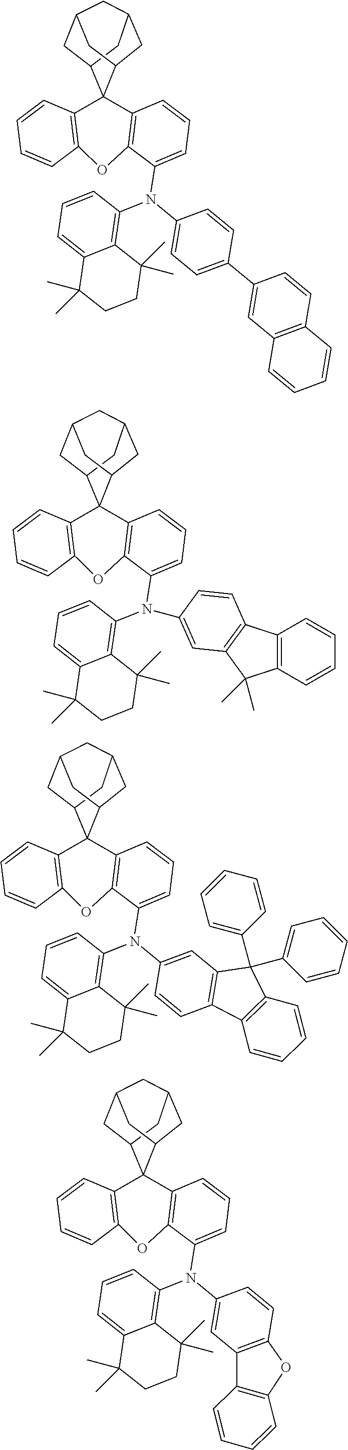

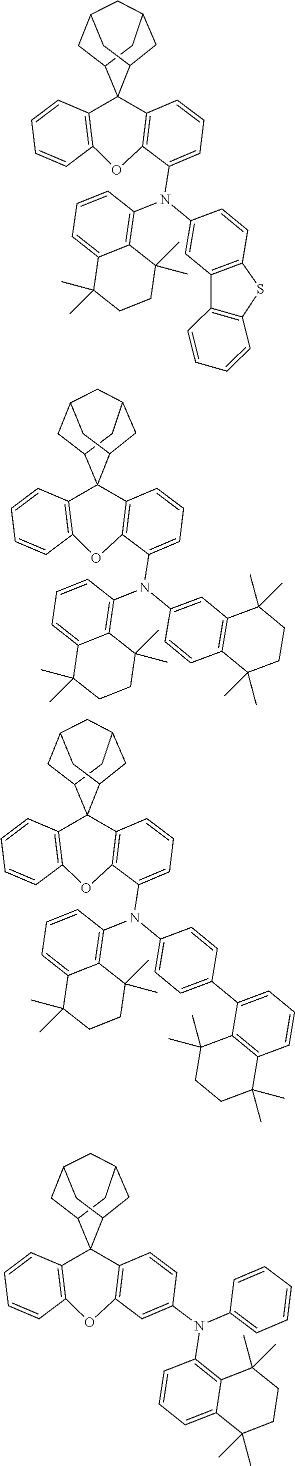

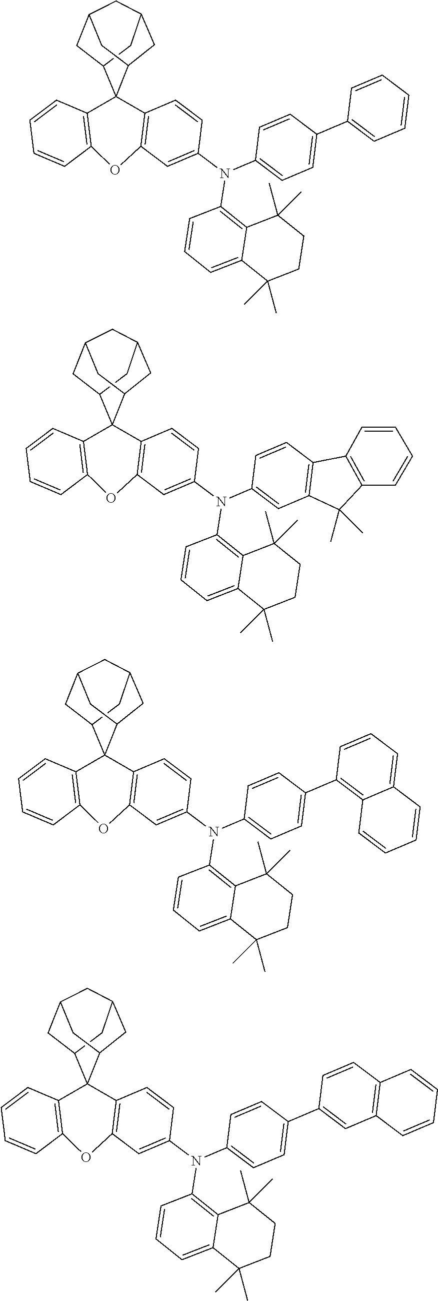 [SMILES: CC1(C)CCC(C)(C)c2c(N(c3ccc(-c4ccccc4)cc3)c3ccc4c(c3)Oc3ccccc3C43C4CC5CC(C4)CC3C5)cccc21 ; CC1(C)CCC(C)(C)c2c(N(c3ccc4c(c3)Oc3ccccc3C43C4CC5CC(C4)CC3C5)c3ccc4c(c3)C(C)(C)c3ccccc3-4)cccc21 ; CC1(C)CCC(C)(C)c2c(N(c3ccc(-c4cccc5ccccc45)cc3)c3ccc4c(c3)Oc3ccccc3C43C4CC5CC(C4)CC3C5)cccc21 ; CC1(C)CCC(C)(C)c2c(N(c3ccc(-c4ccc5ccccc5c4)cc3)c3ccc4c(c3)Oc3ccccc3C43C4CC5CC(C4)CC3C5)cccc21]

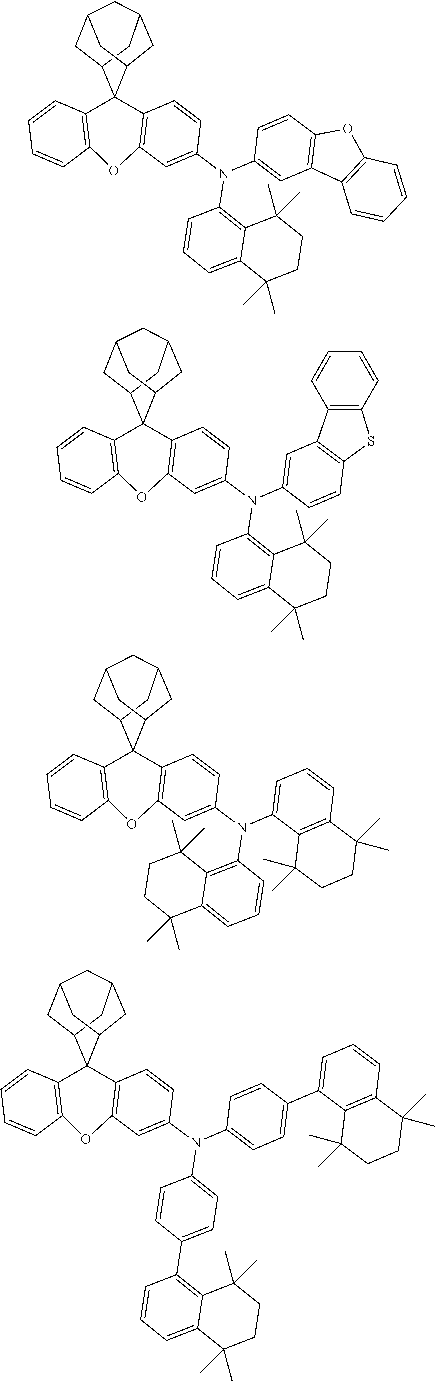 [SMILES: CC1(C)CCC(C)(C)c2c(N(c3ccc4c(c3)Oc3ccccc3C43C4CC5CC(C4)CC3C5)c3ccc4oc5ccccc5c4c3)cccc21 ; CC1(C)CCC(C)(C)c2c(N(c3ccc4c(c3)Oc3ccccc3C43C4CC5CC(C4)CC3C5)c3ccc4sc5ccccc5c4c3)cccc21 ; CC1(C)CCC(C)(C)c2c(N(c3ccc4c(c3)Oc3ccccc3C43C4CC5CC(C4)CC3C5)c3cccc4c3C(C)(C)CCC4(C)C)cccc21 ; CC1(C)CCC(C)(C)c2c(-c3ccc(N(c4ccc(-c5cccc6c5C(C)(C)CCC6(C)C)cc4)c4ccc5c(c4)Oc4ccccc4C54C5CC6CC(C5)CC4C6)cc3)cccc21]

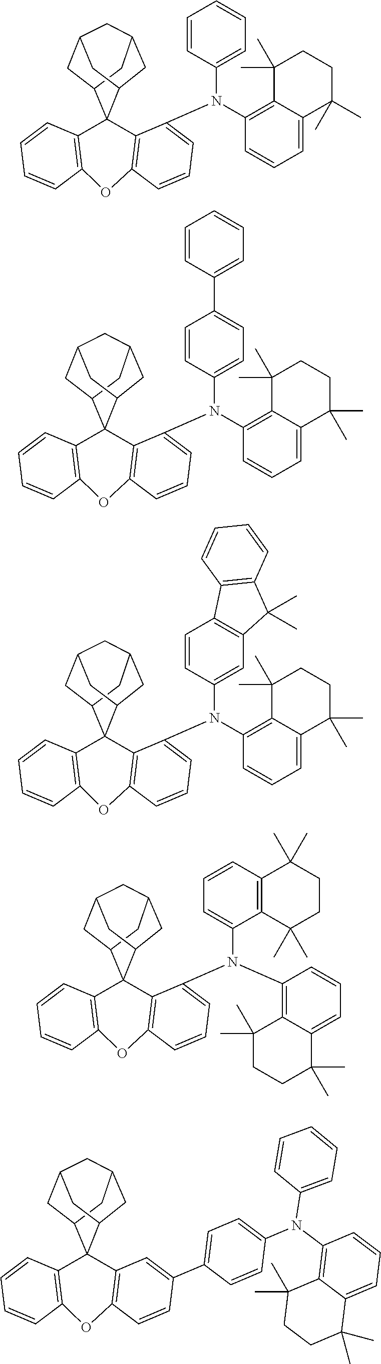

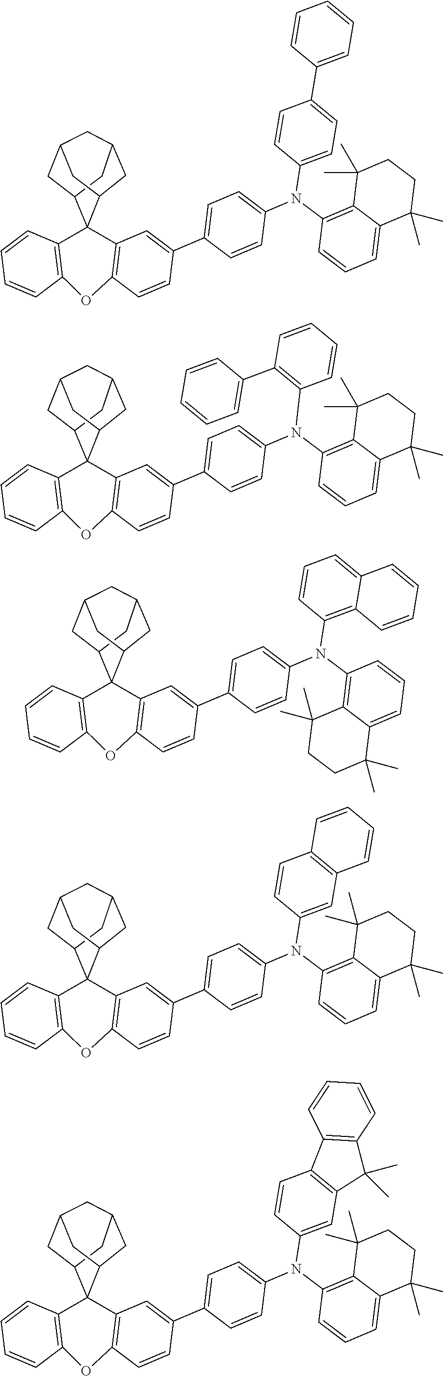

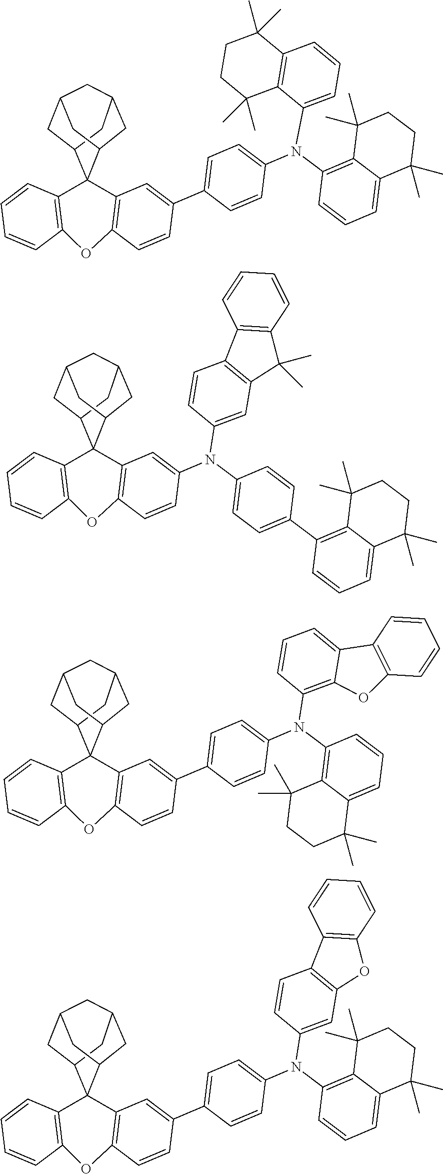

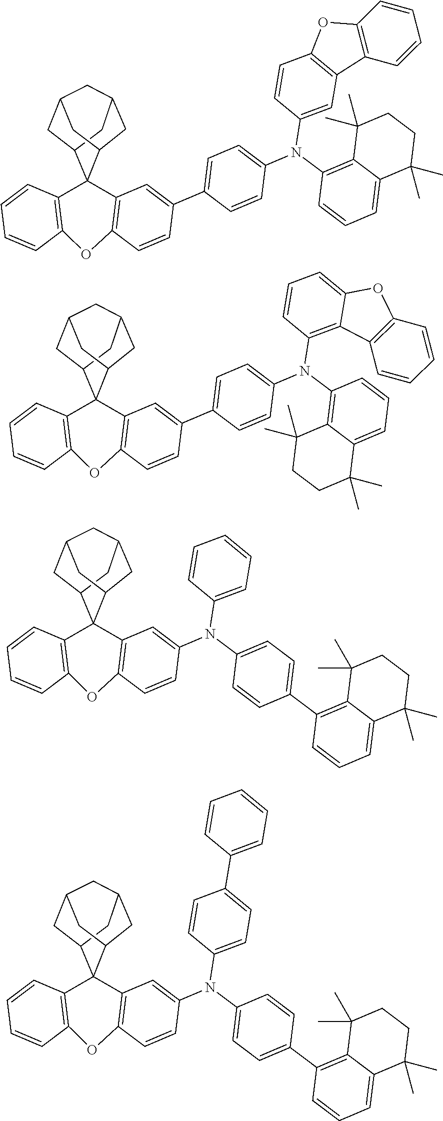 [SMILES: CC1(C)CCC(C)(C)c2c(N(c3ccc(-c4ccc5c(c4)C4(c6ccccc6O5)C5CC6CC(C5)CC4C6)cc3)c3ccc4oc5ccccc5c4c3)cccc21 ; CC1(C)CCC(C)(C)c2c(N(c3ccc(-c4ccc5c(c4)C4(c6ccccc6O5)C5CC6CC(C5)CC4C6)cc3)c3cccc4oc5ccccc5c34)cccc21 ; CC1(C)CCC(C)(C)c2c(-c3ccc(N(c4ccccc4)c4ccc5c(c4)C4(c6ccccc6O5)C5CC6CC(C5)CC4C6)cc3)cccc21 ; CC1(C)CCC(C)(C)c2c(-c3ccc(N(c4ccc(-c5ccccc5)cc4)c4ccc5c(c4)C4(c6ccccc6O5)C5CC6CC(C5)CC4C6)cc3)cccc21]

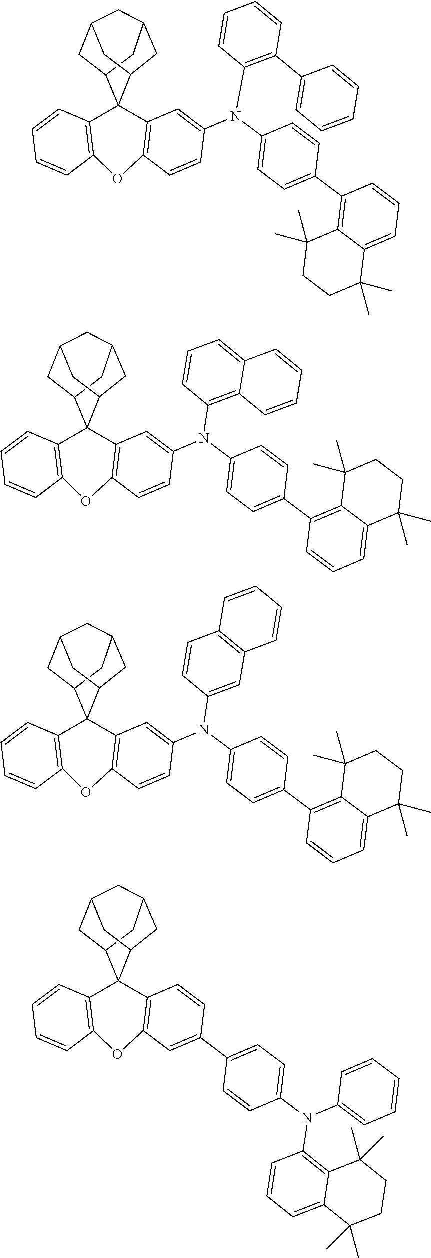 [SMILES: CC1(C)CCC(C)(C)c2c(-c3ccc(N(c4ccc5c(c4)C4(c6ccccc6O5)C5CC6CC(C5)CC4C6)c4ccccc4-c4ccccc4)cc3)cccc21 ; CC1(C)CCC(C)(C)c2c(-c3ccc(N(c4ccc5c(c4)C4(c6ccccc6O5)C5CC6CC(C5)CC4C6)c4cccc5ccccc45)cc3)cccc21 ; CC1(C)CCC(C)(C)c2c(-c3ccc(N(c4ccc5c(c4)C4(c6ccccc6O5)C5CC6CC(C5)CC4C6)c4ccc5ccccc5c4)cc3)cccc21 ; CC1(C)CCC(C)(C)c2c(N(c3ccccc3)c3ccc(-c4ccc5c(c4)Oc4ccccc4C54C5CC6CC(C5)CC4C6)cc3)cccc21]

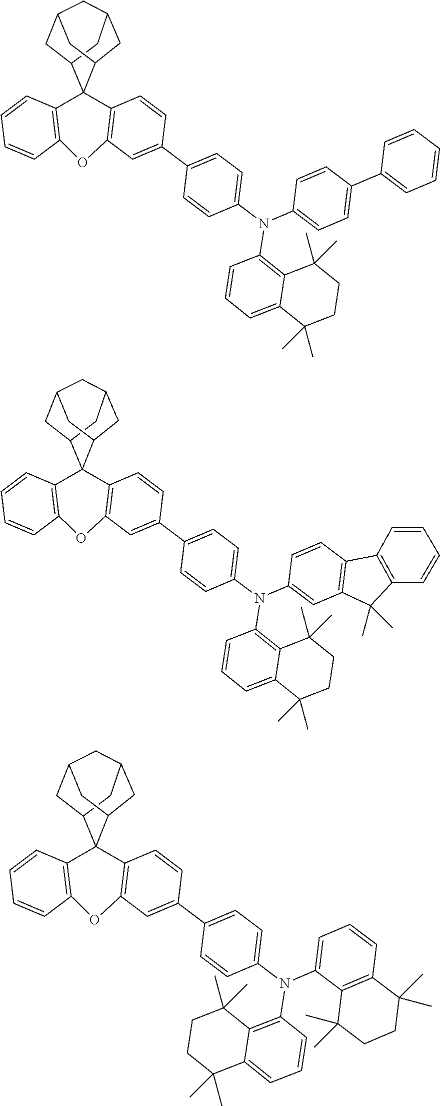 [SMILES: CC1(C)CCC(C)(C)c2c(N(c3ccc(-c4ccccc4)cc3)c3ccc(-c4ccc5c(c4)Oc4ccccc4C54C5CC6CC(C5)CC4C6)cc3)cccc21 ; CC1(C)CCC(C)(C)c2c(N(c3ccc(-c4ccc5c(c4)Oc4ccccc4C54C5CC6CC(C5)CC4C6)cc3)c3ccc4c(c3)C(C)(C)c3ccccc3-4)cccc21 ; CC1(C)CCC(C)(C)c2c(N(c3ccc(-c4ccc5c(c4)Oc4ccccc4C54C5CC6CC(C5)CC4C6)cc3)c3cccc4c3C(C)(C)CCC4(C)C)cccc21]

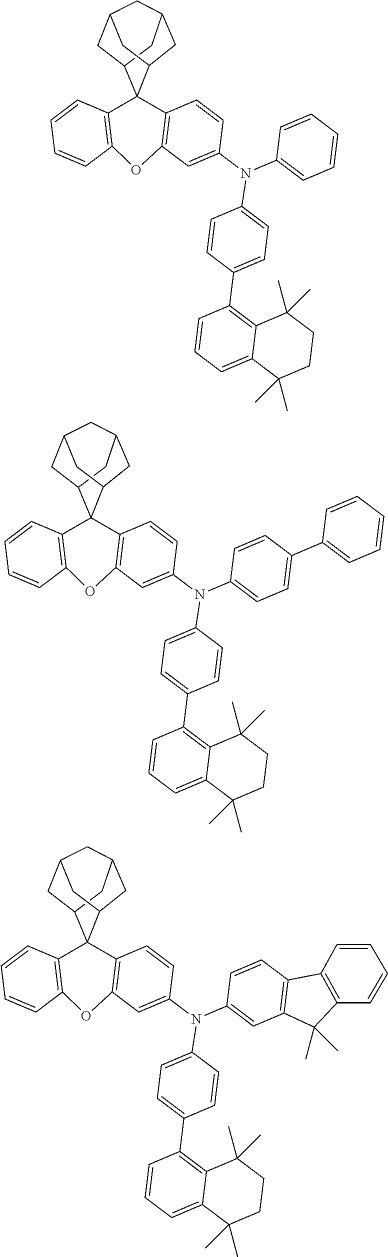 [SMILES: CC1(C)CCC(C)(C)c2c(-c3ccc(N(c4ccccc4)c4ccc5c(c4)Oc4ccccc4C54C5CC6CC(C5)CC4C6)cc3)cccc21 ; CC1(C)CCC(C)(C)c2c(-c3ccc(N(c4ccc(-c5ccccc5)cc4)c4ccc5c(c4)Oc4ccccc4C54C5CC6CC(C5)CC4C6)cc3)cccc21 ; CC1(C)CCC(C)(C)c2c(-c3ccc(N(c4ccc5c(c4)Oc4ccccc4C54C5CC6CC(C5)CC4C6)c4ccc5c(c4)C(C)(C)c4ccccc4-5)cc3)cccc21]

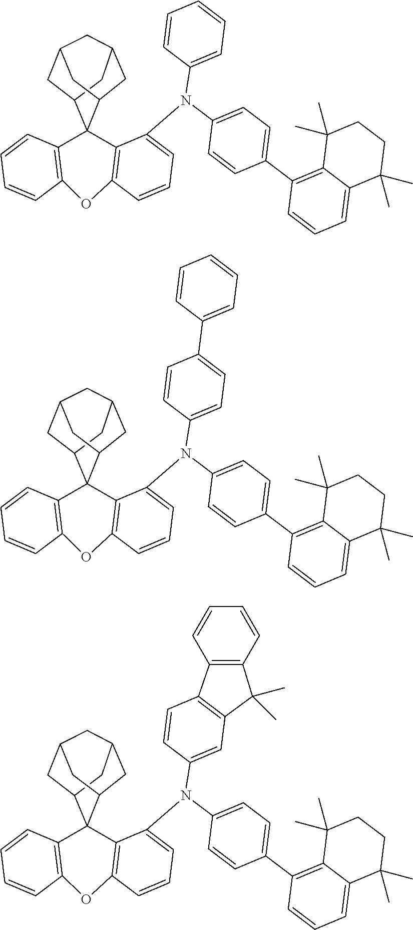 [SMILES: CC1(C)CCC(C)(C)c2c(-c3ccc(N(c4ccccc4)c4cccc5c4C4(c6ccccc6O5)C5CC6CC(C5)CC4C6)cc3)cccc21 ; CC1(C)CCC(C)(C)c2c(-c3ccc(N(c4ccc(-c5ccccc5)cc4)c4cccc5c4C4(c6ccccc6O5)C5CC6CC(C5)CC4C6)cc3)cccc21 ; CC1(C)CCC(C)(C)c2c(-c3ccc(N(c4ccc5c(c4)C(C)(C)c4ccccc4-5)c4cccc5c4C4(c6ccccc6O5)C5CC6CC(C5)CC4C6)cc3)cccc21]

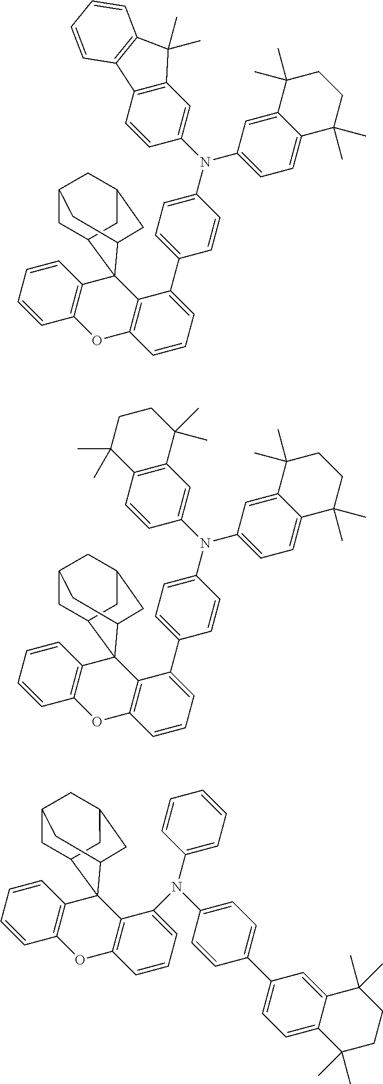 [SMILES: CC1(C)CCC(C)(C)c2cc(N(c3ccc(-c4cccc5c4C4(c6ccccc6O5)C5CC6CC(C5)CC4C6)cc3)c3ccc4c(c3)C(C)(C)c3ccccc3-4)ccc21 ; CC1(C)CCC(C)(C)c2cc(N(c3ccc(-c4cccc5c4C4(c6ccccc6O5)C5CC6CC(C5)CC4C6)cc3)c3ccc4c(c3)C(C)(C)CCC4(C)C)ccc21 ; CC1(C)CCC(C)(C)c2cc(-c3ccc(N(c4ccccc4)c4cccc5c4C4(c6ccccc6O5)C5CC6CC(C5)CC4C6)cc3)ccc21]

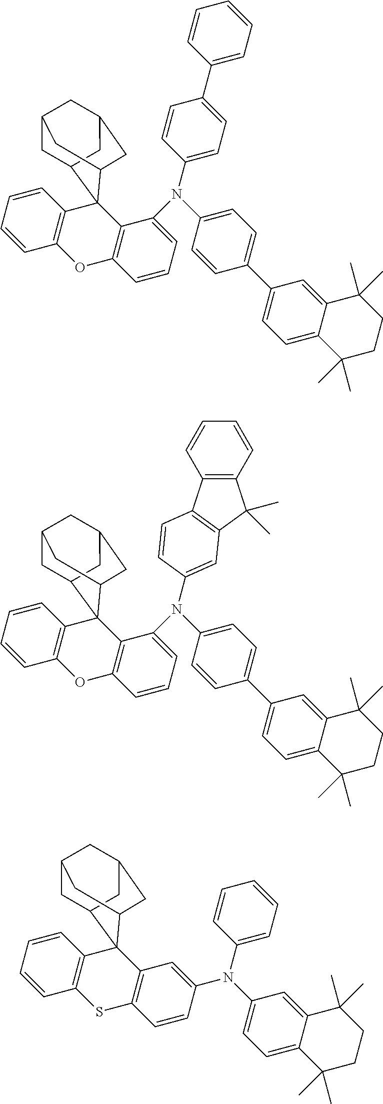 [SMILES: CC1(C)CCC(C)(C)c2cc(-c3ccc(N(c4ccc(-c5ccccc5)cc4)c4cccc5c4C4(c6ccccc6O5)C5CC6CC(C5)CC4C6)cc3)ccc21 ; CC1(C)CCC(C)(C)c2cc(-c3ccc(N(c4ccc5c(c4)C(C)(C)c4ccccc4-5)c4cccc5c4C4(c6ccccc6O5)C5CC6CC(C5)CC4C6)cc3)ccc21 ; CC1(C)CCC(C)(C)c2cc(N(c3ccccc3)c3ccc4c(c3)C3(c5ccccc5S4)C4CC5CC(C4)CC3C5)ccc21]

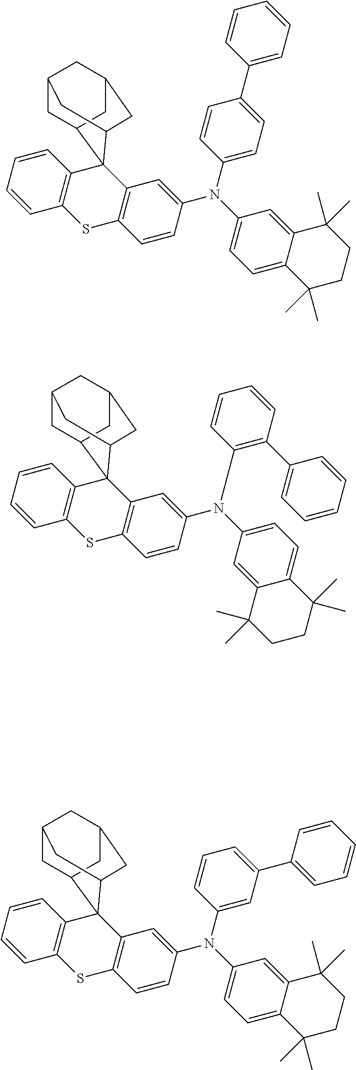 [SMILES: CC1(C)CCC(C)(C)c2cc(N(c3ccc(-c4ccccc4)cc3)c3ccc4c(c3)C3(c5ccccc5S4)C4CC5CC(C4)CC3C5)ccc21 ; CC1(C)CCC(C)(C)c2cc(N(c3ccc4c(c3)C3(c5ccccc5S4)C4CC5CC(C4)CC3C5)c3ccccc3-c3ccccc3)ccc21 ; CC1(C)CCC(C)(C)c2cc(N(c3cccc(-c4ccccc4)c3)c3ccc4c(c3)C3(c5ccccc5S4)C4CC5CC(C4)CC3C5)ccc21]

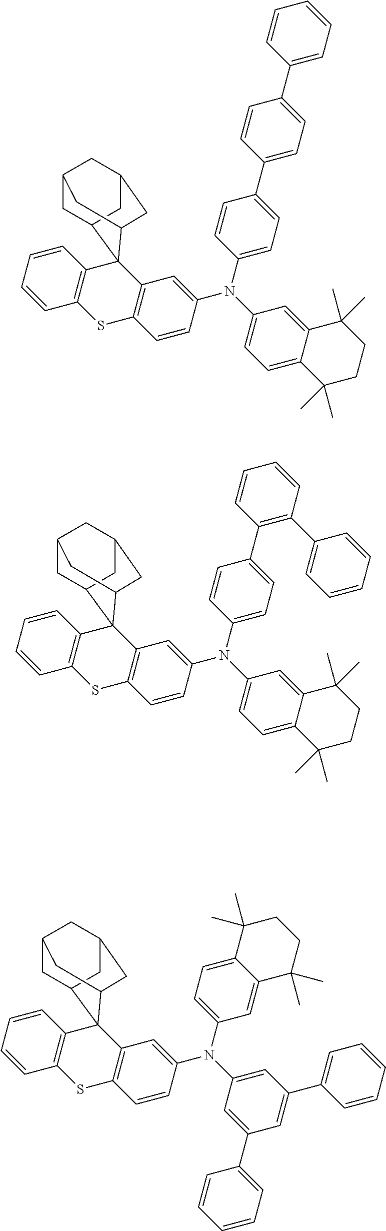 [SMILES: CC1(C)CCC(C)(C)c2cc(N(c3ccc(-c4ccc(-c5ccccc5)cc4)cc3)c3ccc4c(c3)C3(c5ccccc5S4)C4CC5CC(C4)CC3C5)ccc21 ; CC1(C)CCC(C)(C)c2cc(N(c3ccc(-c4ccccc4-c4ccccc4)cc3)c3ccc4c(c3)C3(c5ccccc5S4)C4CC5CC(C4)CC3C5)ccc21 ; CC1(C)CCC(C)(C)c2cc(N(c3cc(-c4ccccc4)cc(-c4ccccc4)c3)c3ccc4c(c3)C3(c5ccccc5S4)C4CC5CC(C4)CC3C5)ccc21]

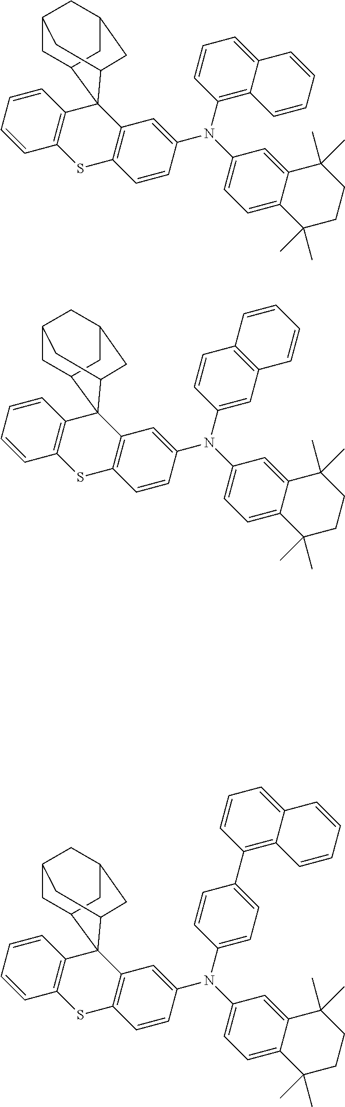 [SMILES: CC1(C)CCC(C)(C)c2cc(N(c3ccc4c(c3)C3(c5ccccc5S4)C4CC5CC(C4)CC3C5)c3cccc4ccccc34)ccc21 ; CC1(C)CCC(C)(C)c2cc(N(c3ccc4c(c3)C3(c5ccccc5S4)C4CC5CC(C4)CC3C5)c3ccc4ccccc4c3)ccc21 ; CC1(C)CCC(C)(C)c2cc(N(c3ccc(-c4cccc5ccccc45)cc3)c3ccc4c(c3)C3(c5ccccc5S4)C4CC5CC(C4)CC3C5)ccc21]

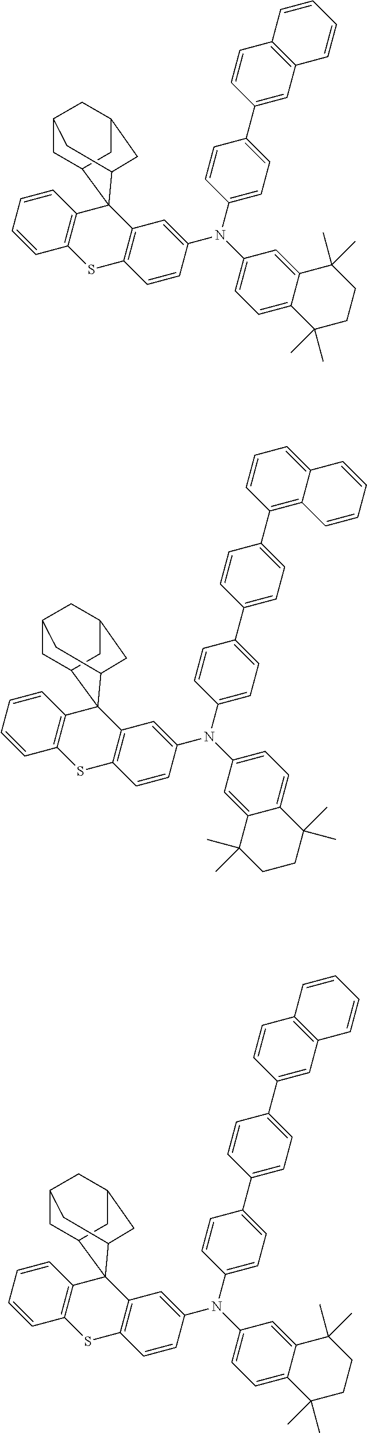 [SMILES: CC1(C)CCC(C)(C)c2cc(N(c3ccc(-c4ccc5ccccc5c4)cc3)c3ccc4c(c3)C3(c5ccccc5S4)C4CC5CC(C4)CC3C5)ccc21 ; CC1(C)CCC(C)(C)c2cc(N(c3ccc(-c4ccc(-c5cccc6ccccc56)cc4)cc3)c3ccc4c(c3)C3(c5ccccc5S4)C4CC5CC(C4)CC3C5)ccc21 ; CC1(C)CCC(C)(C)c2cc(N(c3ccc(-c4ccc(-c5ccc6ccccc6c5)cc4)cc3)c3ccc4c(c3)C3(c5ccccc5S4)C4CC5CC(C4)CC3C5)ccc21]

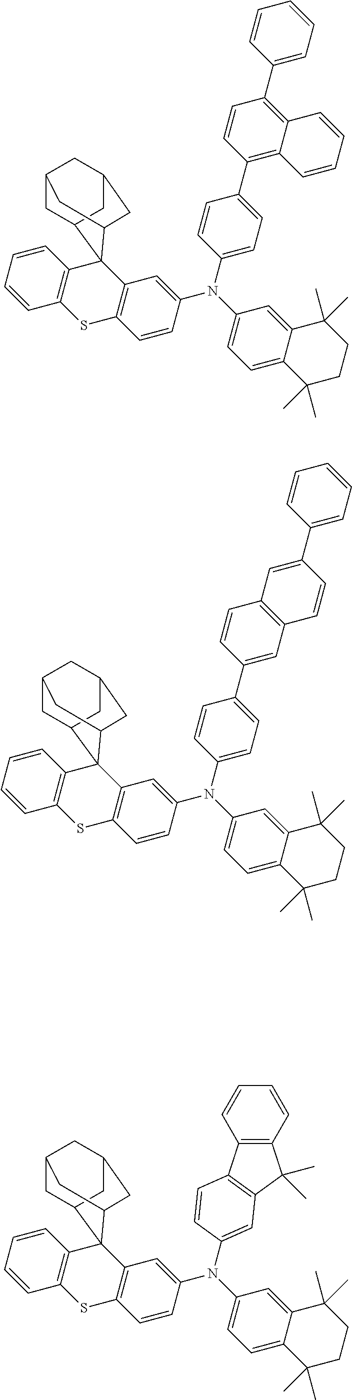 [SMILES: CC1(C)CCC(C)(C)c2cc(N(c3ccc(-c4ccc(-c5ccccc5)c5ccccc45)cc3)c3ccc4c(c3)C3(c5ccccc5S4)C4CC5CC(C4)CC3C5)ccc21 ; CC1(C)CCC(C)(C)c2cc(N(c3ccc(-c4ccc5cc(-c6ccccc6)ccc5c4)cc3)c3ccc4c(c3)C3(c5ccccc5S4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5ccccc5S4)C4CC5CC(C4)CC3C5)ccc21]

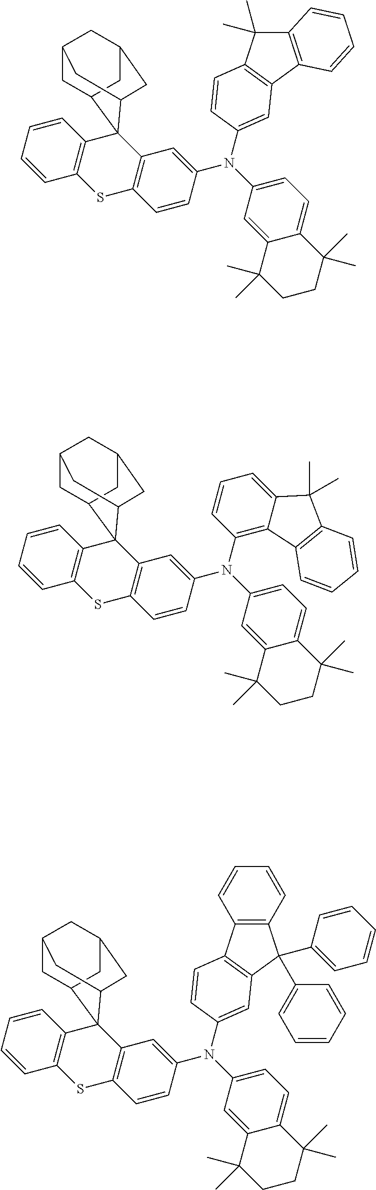 [SMILES: CC1(C)CCC(C)(C)c2cc(N(c3ccc4c(c3)-c3ccccc3C4(C)C)c3ccc4c(c3)C3(c5ccccc5S4)C4CC5CC(C4)CC3C5)ccc21 ; CC1(C)CCC(C)(C)c2cc(N(c3ccc4c(c3)C3(c5ccccc5S4)C4CC5CC(C4)CC3C5)c3cccc4c3-c3ccccc3C4(C)C)ccc21 ; CC1(C)CCC(C)(C)c2cc(N(c3ccc4c(c3)C(c3ccccc3)(c3ccccc3)c3ccccc3-4)c3ccc4c(c3)C3(c5ccccc5S4)C4CC5CC(C4)CC3C5)ccc21]

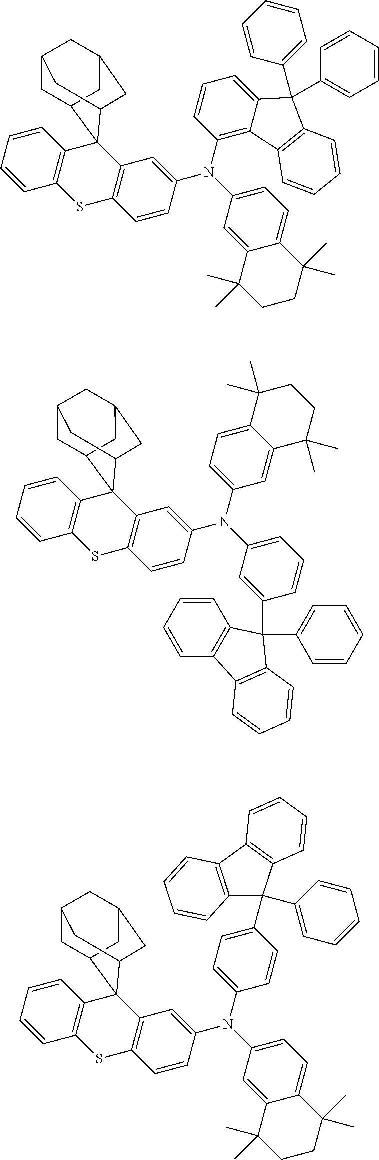 [SMILES: CC1(C)CCC(C)(C)c2cc(N(c3ccc4c(c3)C3(c5ccccc5S4)C4CC5CC(C4)CC3C5)c3cccc4c3-c3ccccc3C4(c3ccccc3)c3ccccc3)ccc21 ; CC1(C)CCC(C)(C)c2cc(N(c3cccc(C4(c5ccccc5)c5ccccc5-c5ccccc54)c3)c3ccc4c(c3)C3(c5ccccc5S4)C4CC5CC(C4)CC3C5)ccc21 ; CC1(C)CCC(C)(C)c2cc(N(c3ccc(C4(c5ccccc5)c5ccccc5-c5ccccc54)cc3)c3ccc4c(c3)C3(c5ccccc5S4)C4CC5CC(C4)CC3C5)ccc21]

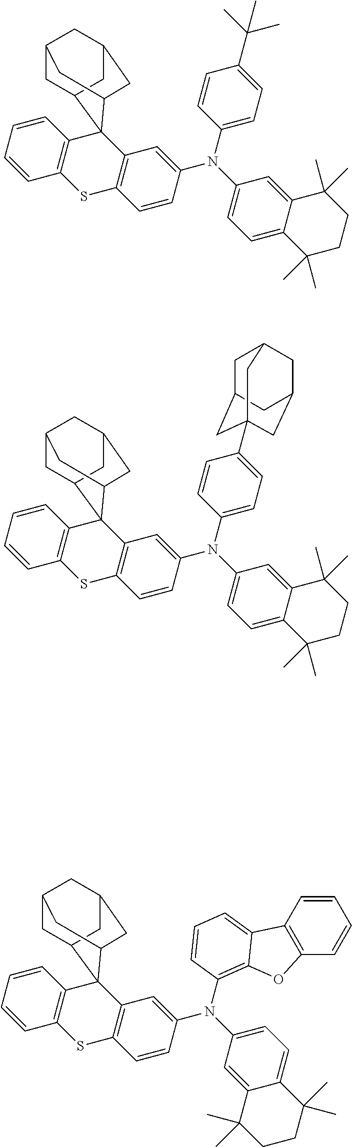 [SMILES: CC(C)(C)c1ccc(N(c2ccc3c(c2)C(C)(C)CCC3(C)C)c2ccc3c(c2)C2(c4ccccc4S3)C3CC4CC(C3)CC2C4)cc1 ; CC1(C)CCC(C)(C)c2cc(N(c3ccc(C45CC6CC(CC(C6)C4)C5)cc3)c3ccc4c(c3)C3(c5ccccc5S4)C4CC5CC(C4)CC3C5)ccc21 ; CC1(C)CCC(C)(C)c2cc(N(c3ccc4c(c3)C3(c5ccccc5S4)C4CC5CC(C4)CC3C5)c3cccc4c3oc3ccccc34)ccc21]

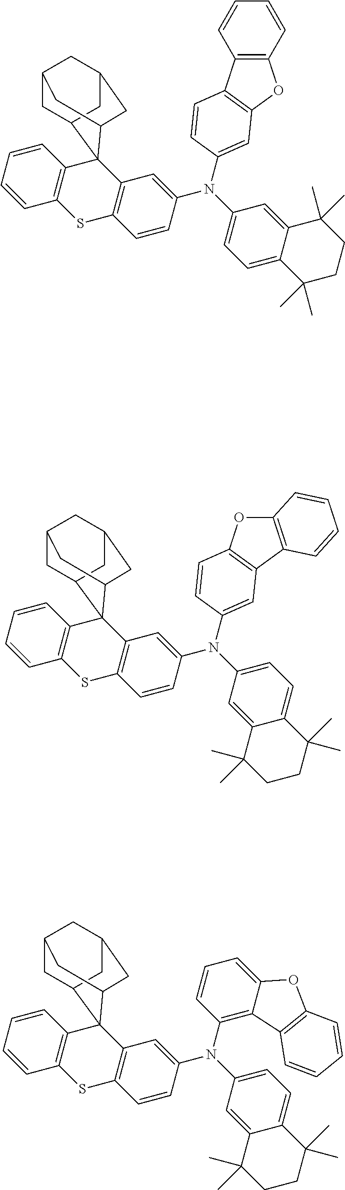 [SMILES: CC1(C)CCC(C)(C)c2cc(N(c3ccc4c(c3)C3(c5ccccc5S4)C4CC5CC(C4)CC3C5)c3ccc4c(c3)oc3ccccc34)ccc21 ; CC1(C)CCC(C)(C)c2cc(N(c3ccc4c(c3)C3(c5ccccc5S4)C4CC5CC(C4)CC3C5)c3ccc4oc5ccccc5c4c3)ccc21 ; CC1(C)CCC(C)(C)c2cc(N(c3ccc4c(c3)C3(c5ccccc5S4)C4CC5CC(C4)CC3C5)c3cccc4oc5ccccc5c34)ccc21]

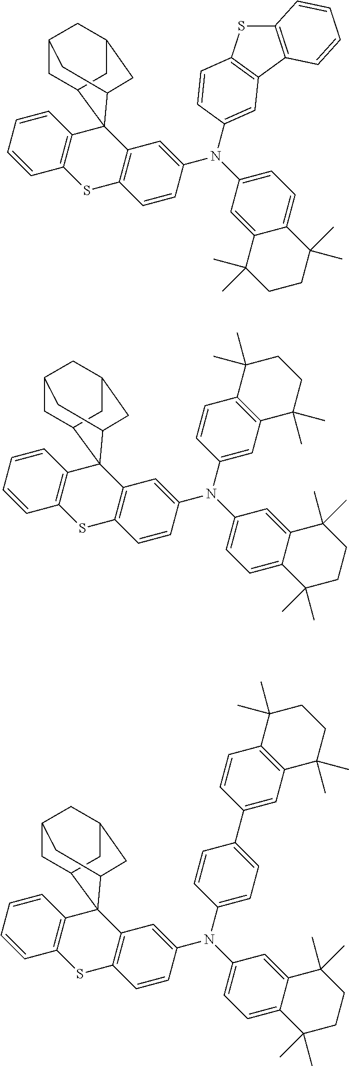

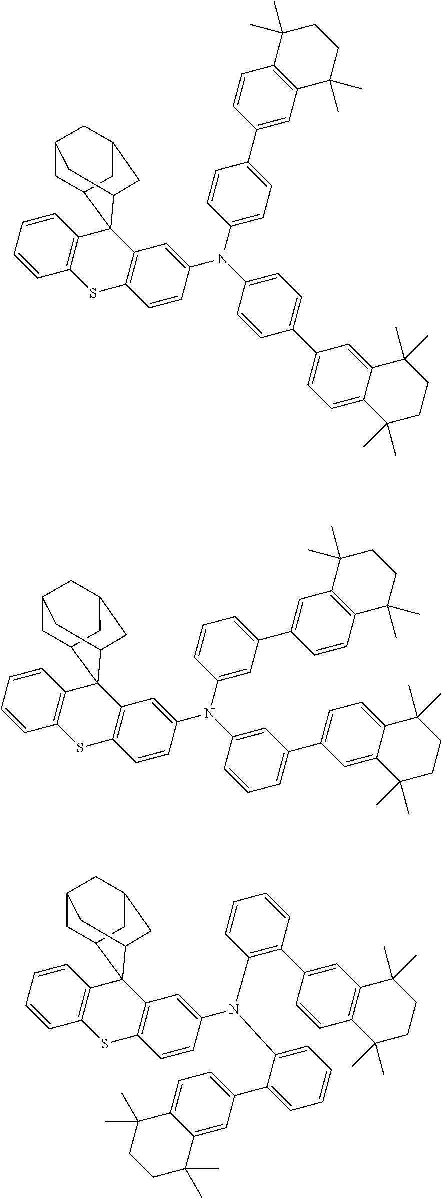 [SMILES: CC1(C)CCC(C)(C)c2cc(-c3ccc(N(c4ccc(-c5ccc6c(c5)C(C)(C)CCC6(C)C)cc4)c4ccc5c(c4)C4(c6ccccc6S5)C5CC6CC(C5)CC4C6)cc3)ccc21 ; CC1(C)CCC(C)(C)c2cc(-c3cccc(N(c4cccc(-c5ccc6c(c5)C(C)(C)CCC6(C)C)c4)c4ccc5c(c4)C4(c6ccccc6S5)C5CC6CC(C5)CC4C6)c3)ccc21 ; CC1(C)CCC(C)(C)c2cc(-c3ccccc3N(c3ccc4c(c3)C3(c5ccccc5S4)C4CC5CC(C4)CC3C5)c3ccccc3-c3ccc4c(c3)C(C)(C)CCC4(C)C)ccc21]

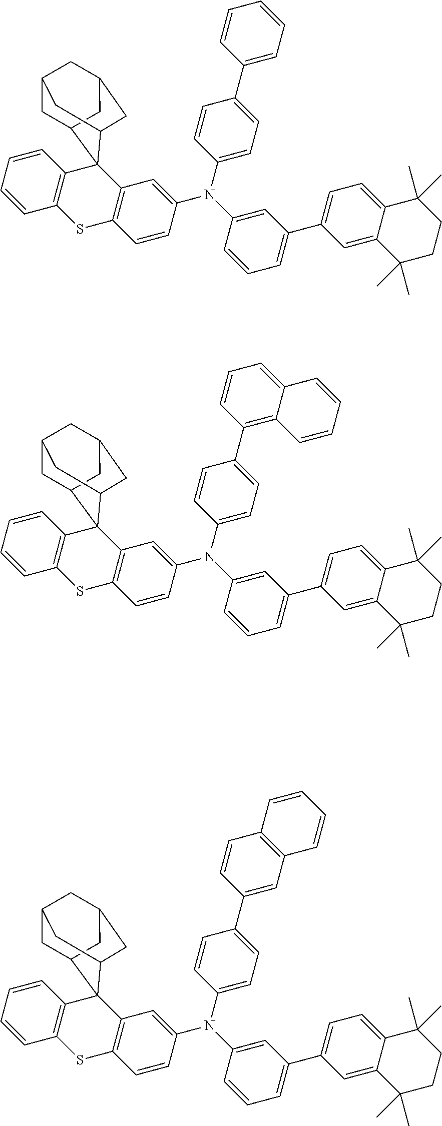 [SMILES: CC1(C)CCC(C)(C)c2cc(-c3cccc(N(c4ccc(-c5ccccc5)cc4)c4ccc5c(c4)C4(c6ccccc6S5)C5CC6CC(C5)CC4C6)c3)ccc21 ; CC1(C)CCC(C)(C)c2cc(-c3cccc(N(c4ccc(-c5cccc6ccccc56)cc4)c4ccc5c(c4)C4(c6ccccc6S5)C5CC6CC(C5)CC4C6)c3)ccc21 ; CC1(C)CCC(C)(C)c2cc(-c3cccc(N(c4ccc(-c5ccc6ccccc6c5)cc4)c4ccc5c(c4)C4(c6ccccc6S5)C5CC6CC(C5)CC4C6)c3)ccc21]

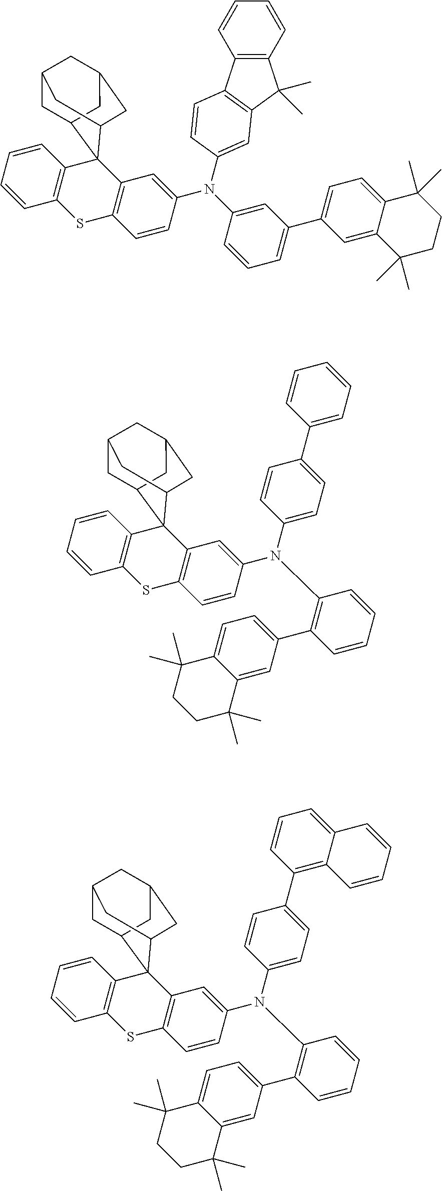 [SMILES: CC1(C)CCC(C)(C)c2cc(-c3cccc(N(c4ccc5c(c4)C(C)(C)c4ccccc4-5)c4ccc5c(c4)C4(c6ccccc6S5)C5CC6CC(C5)CC4C6)c3)ccc21 ; CC1(C)CCC(C)(C)c2cc(-c3ccccc3N(c3ccc(-c4ccccc4)cc3)c3ccc4c(c3)C3(c5ccccc5S4)C4CC5CC(C4)CC3C5)ccc21 ; CC1(C)CCC(C)(C)c2cc(-c3ccccc3N(c3ccc(-c4cccc5ccccc45)cc3)c3ccc4c(c3)C3(c5ccccc5S4)C4CC5CC(C4)CC3C5)ccc21]

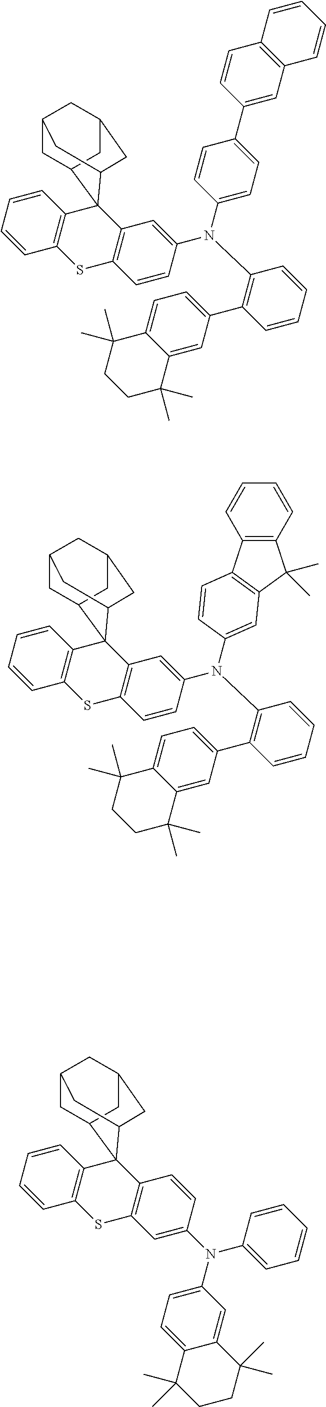 [SMILES: CC1(C)CCC(C)(C)c2cc(-c3ccccc3N(c3ccc(-c4ccc5ccccc5c4)cc3)c3ccc4c(c3)C3(c5ccccc5S4)C4CC5CC(C4)CC3C5)ccc21 ; CC1(C)CCC(C)(C)c2cc(-c3ccccc3N(c3ccc4c(c3)C(C)(C)c3ccccc3-4)c3ccc4c(c3)C3(c5ccccc5S4)C4CC5CC(C4)CC3C5)ccc21 ; CC1(C)CCC(C)(C)c2cc(N(c3ccccc3)c3ccc4c(c3)Sc3ccccc3C43C4CC5CC(C4)CC3C5)ccc21]

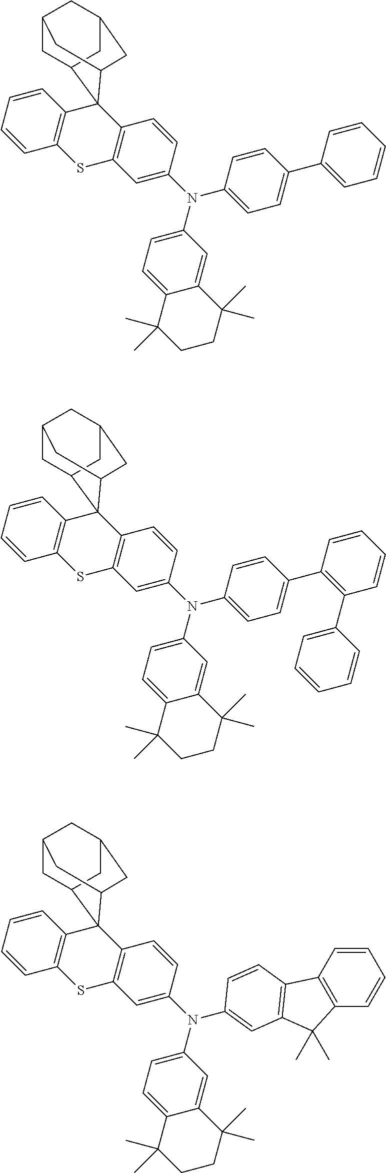 [SMILES: CC1(C)CCC(C)(C)c2cc(N(c3ccc(-c4ccccc4)cc3)c3ccc4c(c3)Sc3ccccc3C43C4CC5CC(C4)CC3C5)ccc21 ; CC1(C)CCC(C)(C)c2cc(N(c3ccc(-c4ccccc4-c4ccccc4)cc3)c3ccc4c(c3)Sc3ccccc3C43C4CC5CC(C4)CC3C5)ccc21 ; CC1(C)CCC(C)(C)c2cc(N(c3ccc4c(c3)Sc3ccccc3C43C4CC5CC(C4)CC3C5)c3ccc4c(c3)C(C)(C)c3ccccc3-4)ccc21]

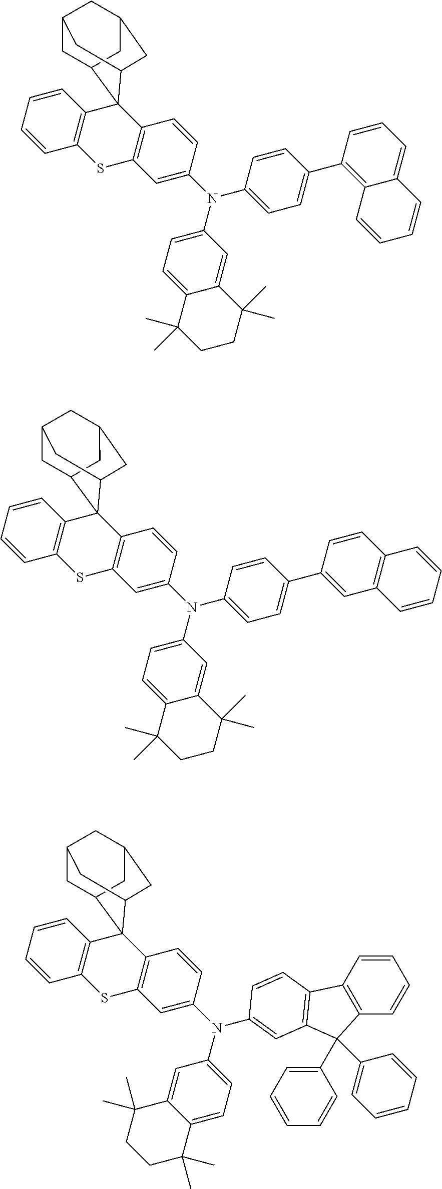 [SMILES: CC1(C)CCC(C)(C)c2cc(N(c3ccc(-c4cccc5ccccc45)cc3)c3ccc4c(c3)Sc3ccccc3C43C4CC5CC(C4)CC3C5)ccc21 ; CC1(C)CCC(C)(C)c2cc(N(c3ccc(-c4ccc5ccccc5c4)cc3)c3ccc4c(c3)Sc3ccccc3C43C4CC5CC(C4)CC3C5)ccc21 ; CC1(C)CCC(C)(C)c2cc(N(c3ccc4c(c3)Sc3ccccc3C43C4CC5CC(C4)CC3C5)c3ccc4c(c3)C(c3ccccc3)(c3ccccc3)c3ccccc3-4)ccc21]

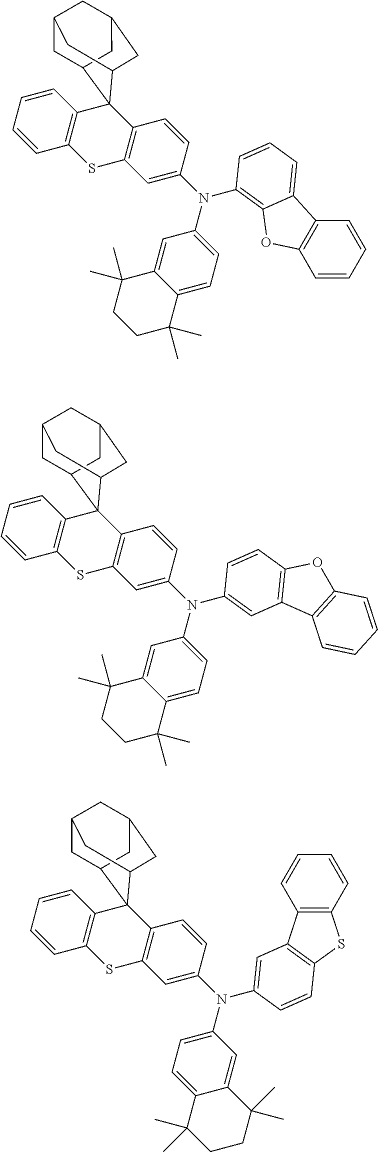 [SMILES: CC1(C)CCC(C)(C)c2cc(N(c3ccc4c(c3)Sc3ccccc3C43C4CC5CC(C4)CC3C5)c3cccc4c3oc3ccccc34)ccc21 ; CC1(C)CCC(C)(C)c2cc(N(c3ccc4c(c3)Sc3ccccc3C43C4CC5CC(C4)CC3C5)c3ccc4oc5ccccc5c4c3)ccc21 ; CC1(C)CCC(C)(C)c2cc(N(c3ccc4c(c3)Sc3ccccc3C43C4CC5CC(C4)CC3C5)c3ccc4sc5ccccc5c4c3)ccc21]

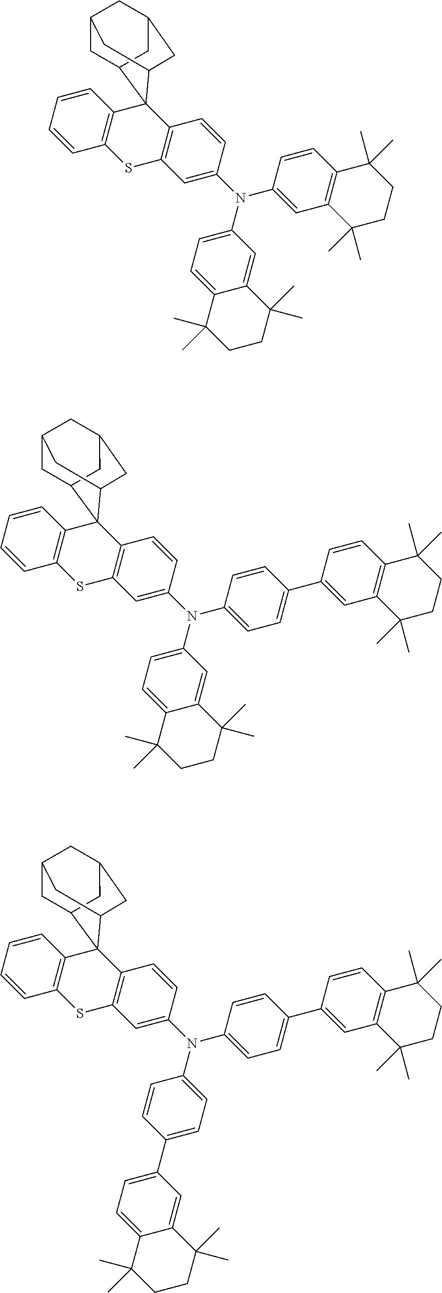 [SMILES: CC1(C)CCC(C)(C)c2cc(N(c3ccc4c(c3)Sc3ccccc3C43C4CC5CC(C4)CC3C5)c3ccc4c(c3)C(C)(C)CCC4(C)C)ccc21 ; CC1(C)CCC(C)(C)c2cc(-c3ccc(N(c4ccc5c(c4)Sc4ccccc4C54C5CC6CC(C5)CC4C6)c4ccc5c(c4)C(C)(C)CCC5(C)C)cc3)ccc21 ; CC1(C)CCC(C)(C)c2cc(-c3ccc(N(c4ccc(-c5ccc6c(c5)C(C)(C)CCC6(C)C)cc4)c4ccc5c(c4)Sc4ccccc4C54C5CC6CC(C5)CC4C6)cc3)ccc21]

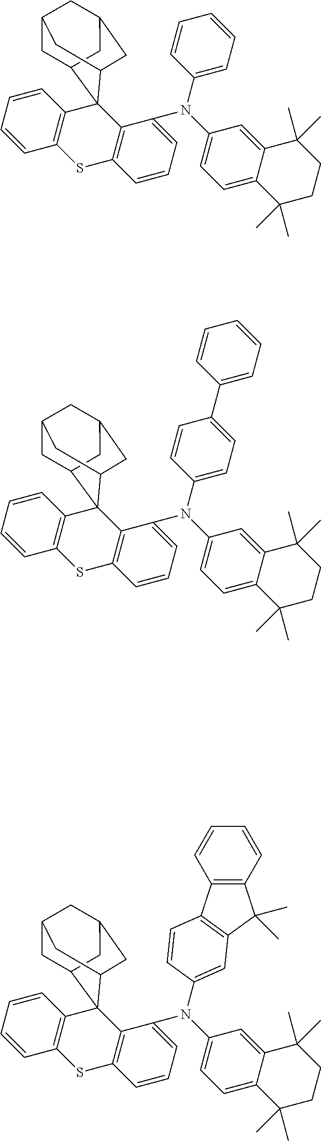 [SMILES: CC1(C)CCC(C)(C)c2cc(N(c3ccccc3)c3cccc4c3C3(c5ccccc5S4)C4CC5CC(C4)CC3C5)ccc21 ; CC1(C)CCC(C)(C)c2cc(N(c3ccc(-c4ccccc4)cc3)c3cccc4c3C3(c5ccccc5S4)C4CC5CC(C4)CC3C5)ccc21 ; CC1(C)CCC(C)(C)c2cc(N(c3ccc4c(c3)C(C)(C)c3ccccc3-4)c3cccc4c3C3(c5ccccc5S4)C4CC5CC(C4)CC3C5)ccc21]

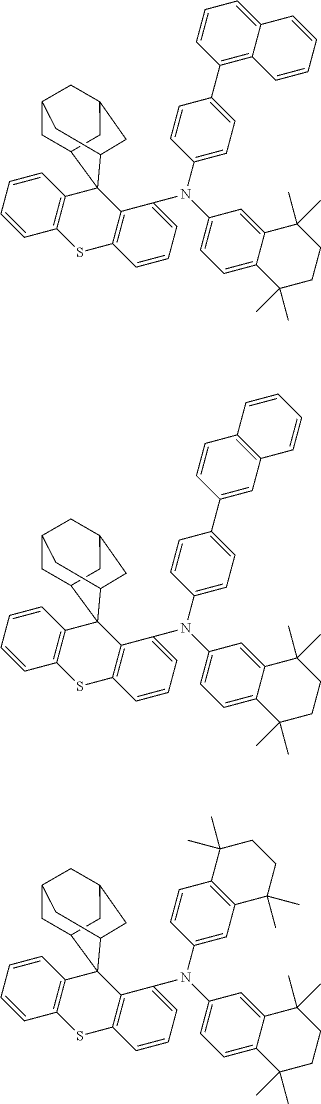 [SMILES: CC1(C)CCC(C)(C)c2cc(N(c3ccc(-c4cccc5ccccc45)cc3)c3cccc4c3C3(c5ccccc5S4)C4CC5CC(C4)CC3C5)ccc21 ; CC1(C)CCC(C)(C)c2cc(N(c3ccc(-c4ccc5ccccc5c4)cc3)c3cccc4c3C3(c5ccccc5S4)C4CC5CC(C4)CC3C5)ccc21 ; CC1(C)CCC(C)(C)c2cc(N(c3ccc4c(c3)C(C)(C)CCC4(C)C)c3cccc4c3C3(c5ccccc5S4)C4CC5CC(C4)CC3C5)ccc21]

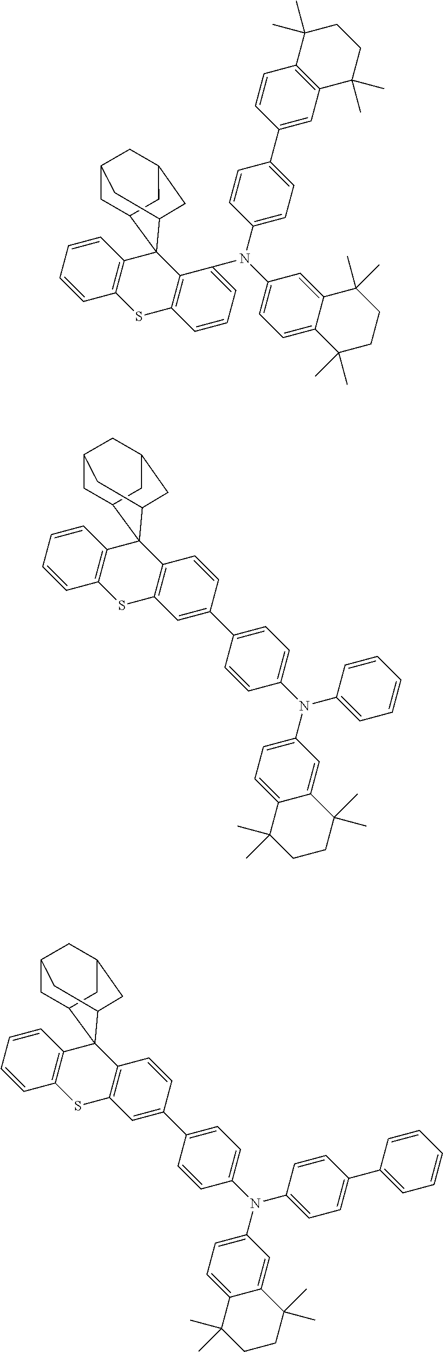 [SMILES: CC1(C)CCC(C)(C)c2cc(-c3ccc(N(c4ccc5c(c4)C(C)(C)CCC5(C)C)c4cccc5c4C4(c6ccccc6S5)C5CC6CC(C5)CC4C6)cc3)ccc21 ; CC1(C)CCC(C)(C)c2cc(N(c3ccccc3)c3ccc(-c4ccc5c(c4)Sc4ccccc4C54C5CC6CC(C5)CC4C6)cc3)ccc21 ; CC1(C)CCC(C)(C)c2cc(N(c3ccc(-c4ccccc4)cc3)c3ccc(-c4ccc5c(c4)Sc4ccccc4C54C5CC6CC(C5)CC4C6)cc3)ccc21]

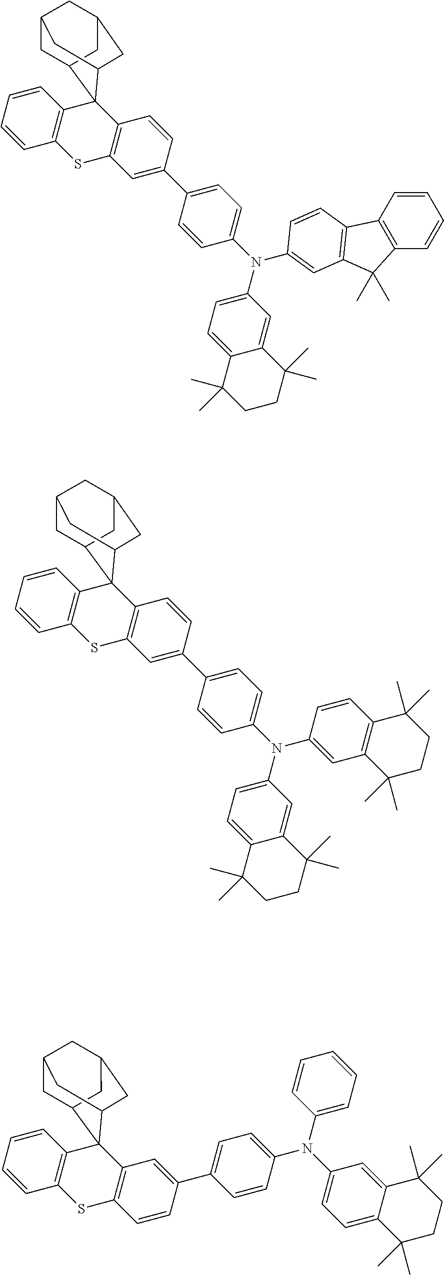 [SMILES: CC1(C)CCC(C)(C)c2cc(N(c3ccc(-c4ccc5c(c4)Sc4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc5c(c4)Sc4ccccc4C54C5CC6CC(C5)CC4C6)cc3)c3ccc4c(c3)C(C)(C)CCC4(C)C)ccc21 ; CC1(C)CCC(C)(C)c2cc(N(c3ccccc3)c3ccc(-c4ccc5c(c4)C4(c6ccccc6S5)C5CC6CC(C5)CC4C6)cc3)ccc21]

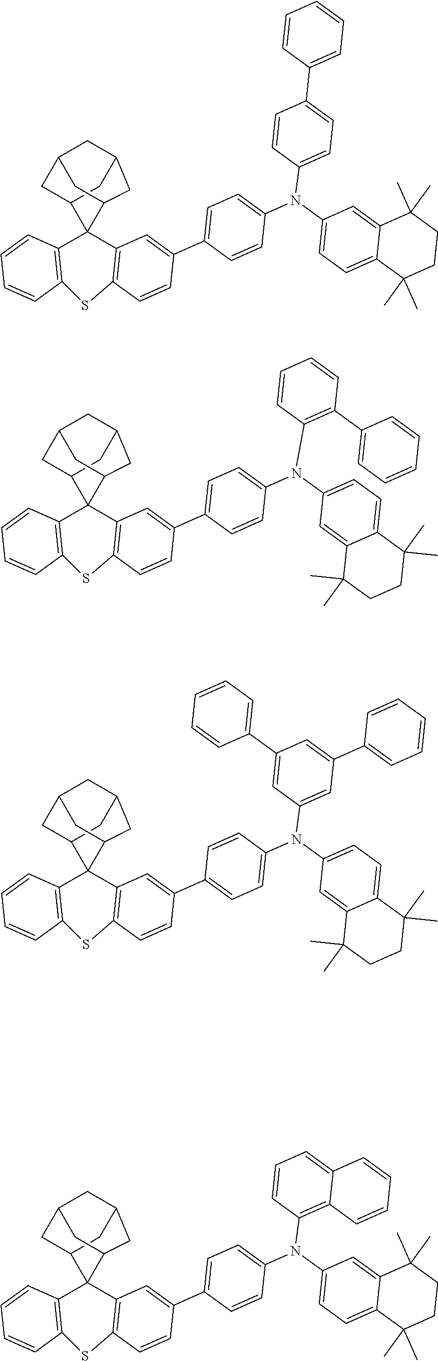 [SMILES: CC1(C)CCC(C)(C)c2cc(N(c3ccc(-c4ccccc4)cc3)c3ccc(-c4ccc5c(c4)C4(c6ccccc6S5)C5CC6CC(C5)CC4C6)cc3)ccc21 ; CC1(C)CCC(C)(C)c2cc(N(c3ccc(-c4ccc5c(c4)C4(c6ccccc6S5)C5CC6CC(C5)CC4C6)cc3)c3ccccc3-c3ccccc3)ccc21 ; CC1(C)CCC(C)(C)c2cc(N(c3ccc(-c4ccc5c(c4)C4(c6ccccc6S5)C5CC6CC(C5)CC4C6)cc3)c3cc(-c4ccccc4)cc(-c4ccccc4)c3)ccc21 ; CC1(C)CCC(C)(C)c2cc(N(c3ccc(-c4ccc5c(c4)C4(c6ccccc6S5)C5CC6CC(C5)CC4C6)cc3)c3cccc4ccccc34)ccc21]

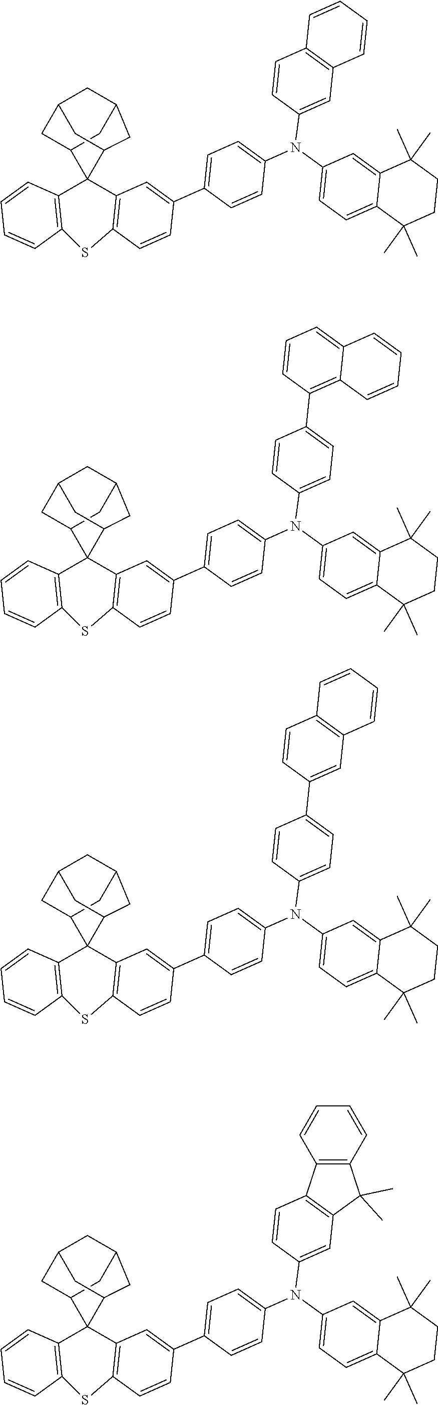 [SMILES: CC1(C)CCC(C)(C)c2cc(N(c3ccc(-c4ccc5c(c4)C4(c6ccccc6S5)C5CC6CC(C5)CC4C6)cc3)c3ccc4ccccc4c3)ccc21 ; CC1(C)CCC(C)(C)c2cc(N(c3ccc(-c4ccc5c(c4)C4(c6ccccc6S5)C5CC6CC(C5)CC4C6)cc3)c3ccc(-c4cccc5ccccc45)cc3)ccc21 ; CC1(C)CCC(C)(C)c2cc(N(c3ccc(-c4ccc5c(c4)C4(c6ccccc6S5)C5CC6CC(C5)CC4C6)cc3)c3ccc(-c4ccc5ccccc5c4)cc3)ccc21 ; CC1(C)CCC(C)(C)c2cc(N(c3ccc(-c4ccc5c(c4)C4(c6ccccc6S5)C5CC6CC(C5)CC4C6)cc3)c3ccc4c(c3)C(C)(C)c3ccccc3-4)ccc21]

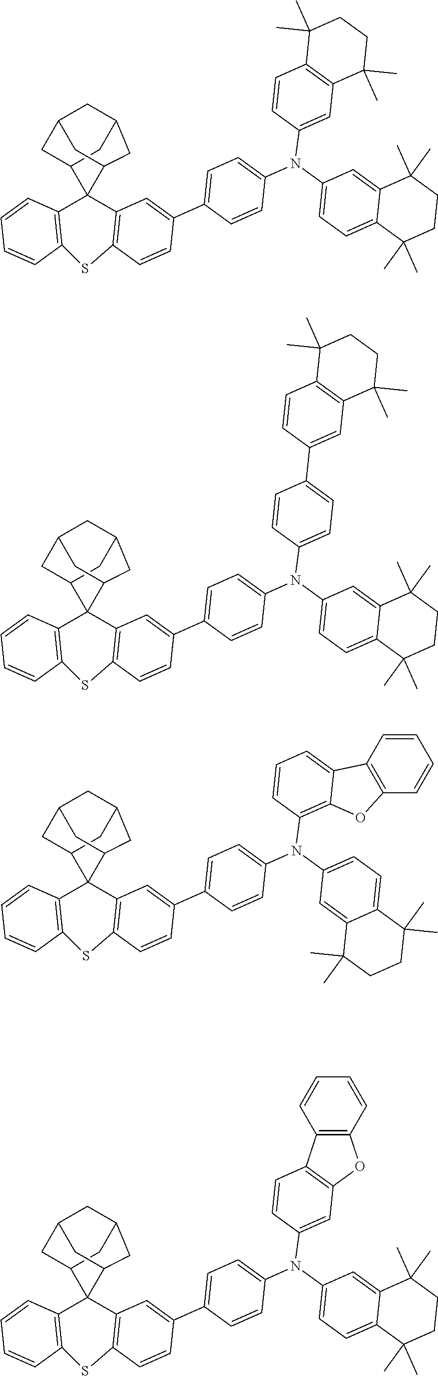

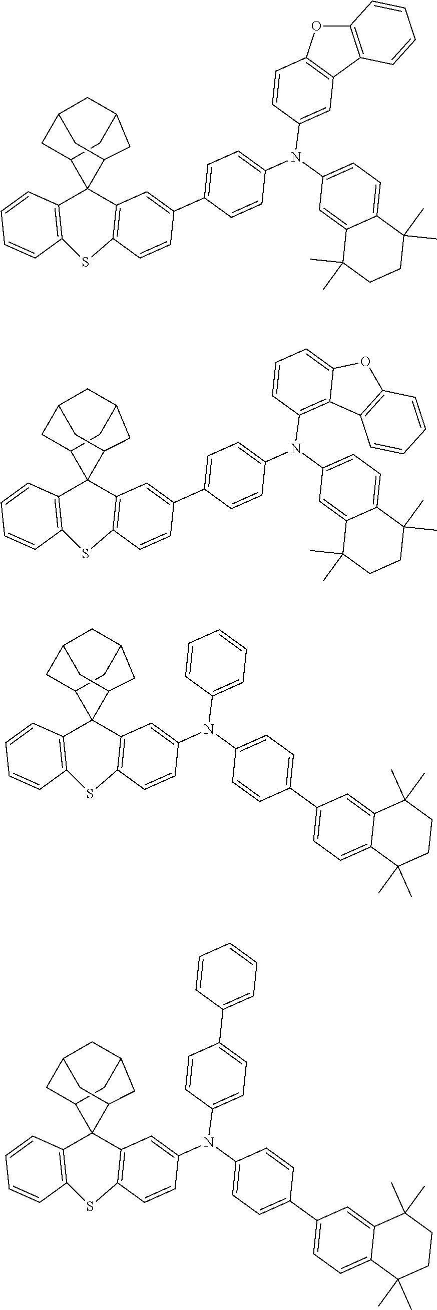 [SMILES: CC1(C)CCC(C)(C)c2cc(N(c3ccc(-c4ccc5c(c4)C4(c6ccccc6S5)C5CC6CC(C5)CC4C6)cc3)c3ccc4oc5ccccc5c4c3)ccc21 ; CC1(C)CCC(C)(C)c2cc(N(c3ccc(-c4ccc5c(c4)C4(c6ccccc6S5)C5CC6CC(C5)CC4C6)cc3)c3cccc4oc5ccccc5c34)ccc21 ; CC1(C)CCC(C)(C)c2cc(-c3ccc(N(c4ccccc4)c4ccc5c(c4)C4(c6ccccc6S5)C5CC6CC(C5)CC4C6)cc3)ccc21 ; CC1(C)CCC(C)(C)c2cc(-c3ccc(N(c4ccc(-c5ccccc5)cc4)c4ccc5c(c4)C4(c6ccccc6S5)C5CC6CC(C5)CC4C6)cc3)ccc21]

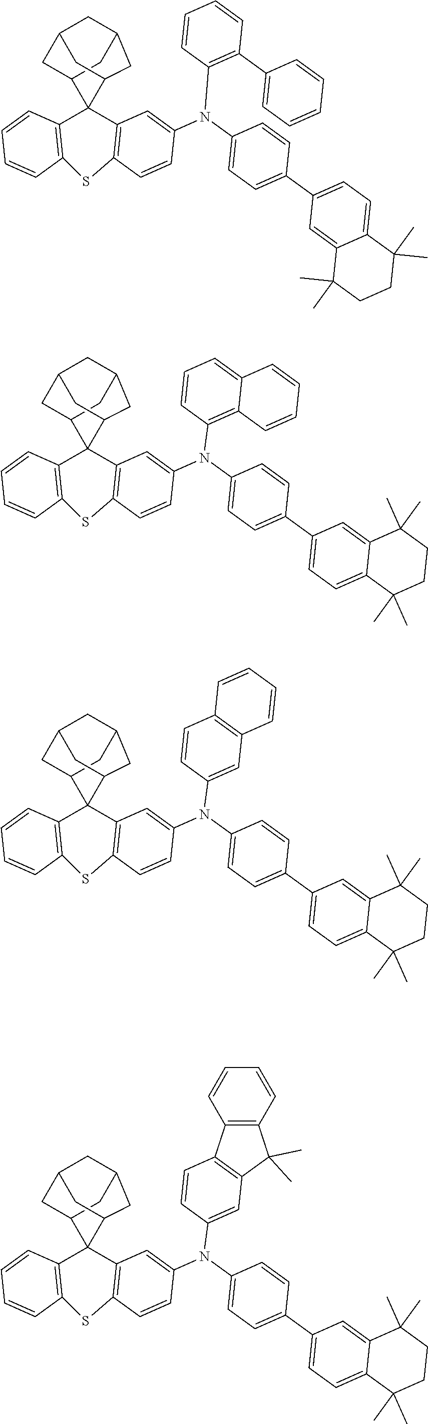 [SMILES: CC1(C)CCC(C)(C)c2cc(-c3ccc(N(c4ccc5c(c4)C4(c6ccccc6S5)C5CC6CC(C5)CC4C6)c4ccccc4-c4ccccc4)cc3)ccc21 ; CC1(C)CCC(C)(C)c2cc(-c3ccc(N(c4ccc5c(c4)C4(c6ccccc6S5)C5CC6CC(C5)CC4C6)c4cccc5ccccc45)cc3)ccc21 ; CC1(C)CCC(C)(C)c2cc(-c3ccc(N(c4ccc5c(c4)C4(c6ccccc6S5)C5CC6CC(C5)CC4C6)c4ccc5ccccc5c4)cc3)ccc21 ; CC1(C)CCC(C)(C)c2cc(-c3ccc(N(c4ccc5c(c4)C(C)(C)c4ccccc4-5)c4ccc5c(c4)C4(c6ccccc6S5)C5CC6CC(C5)CC4C6)cc3)ccc21]

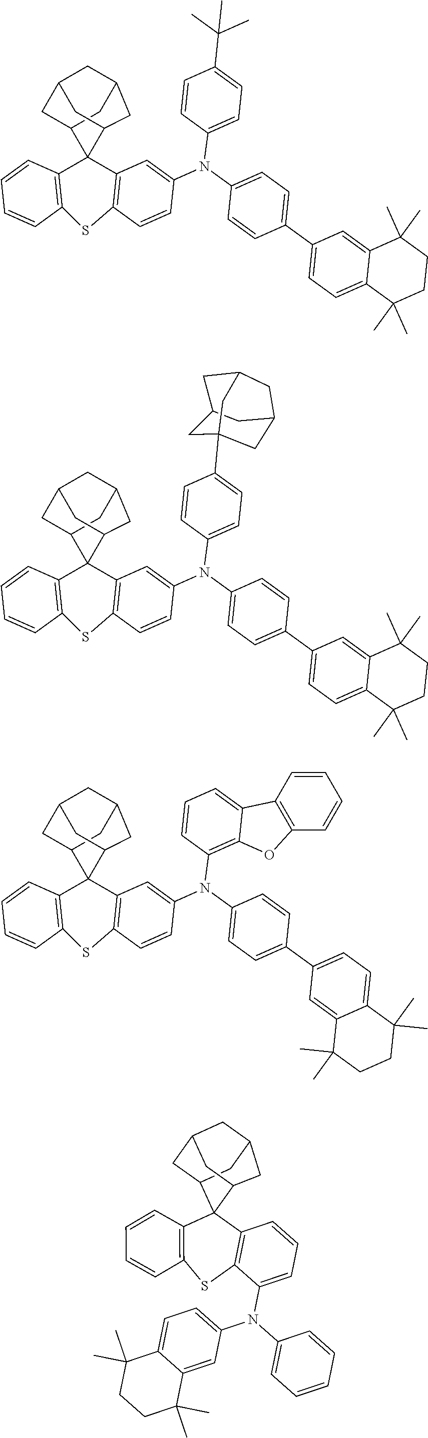

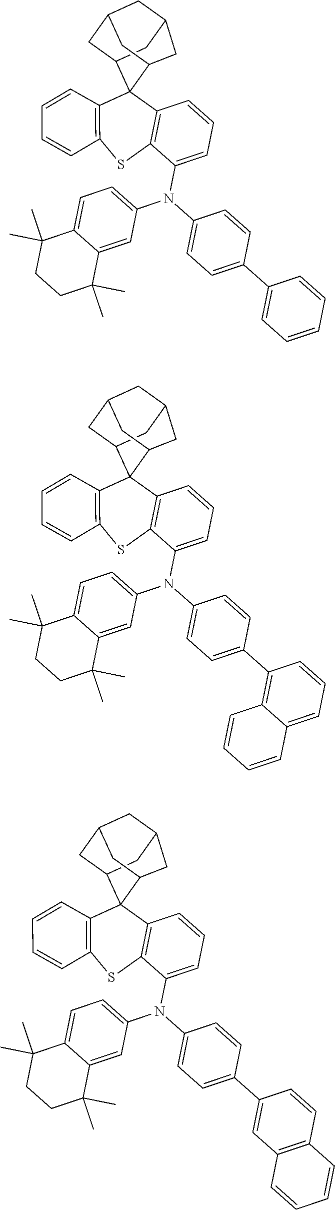 [SMILES: CC1(C)CCC(C)(C)c2cc(N(c3ccc(-c4ccccc4)cc3)c3cccc4c3Sc3ccccc3C43C4CC5CC(C4)CC3C5)ccc21 ; CC1(C)CCC(C)(C)c2cc(N(c3ccc(-c4cccc5ccccc45)cc3)c3cccc4c3Sc3ccccc3C43C4CC5CC(C4)CC3C5)ccc21 ; CC1(C)CCC(C)(C)c2cc(N(c3ccc(-c4ccc5ccccc5c4)cc3)c3cccc4c3Sc3ccccc3C43C4CC5CC(C4)CC3C5)ccc21]

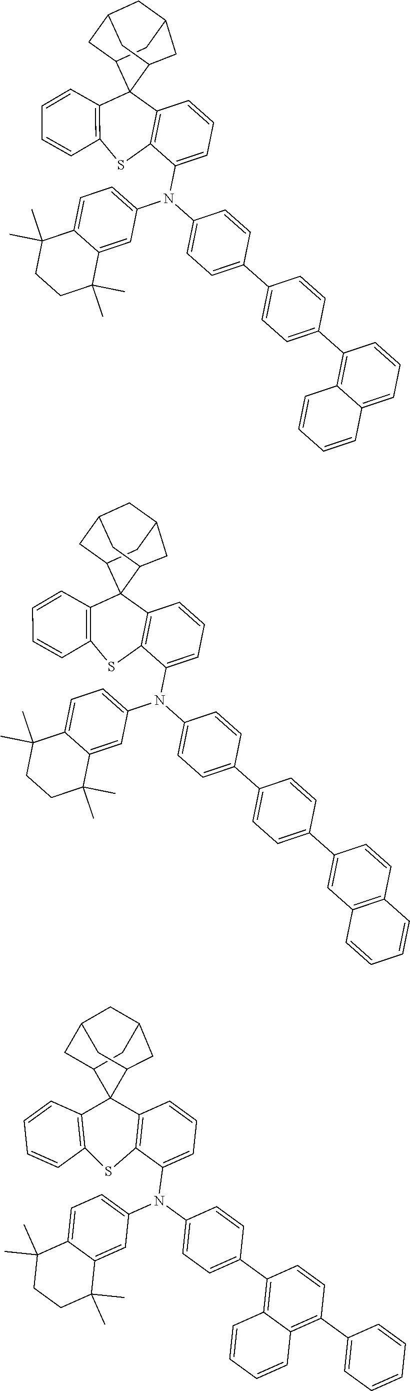 [SMILES: CC1(C)CCC(C)(C)c2cc(N(c3ccc(-c4ccc(-c5cccc6ccccc56)cc4)cc3)c3cccc4c3Sc3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3cccc4c3Sc3ccccc3C43C4CC5CC(C4)CC3C5)ccc21 ; CC1(C)CCC(C)(C)c2cc(N(c3ccc(-c4ccc(-c5ccccc5)c5ccccc45)cc3)c3cccc4c3Sc3ccccc3C43C4CC5CC(C4)CC3C5)ccc21]

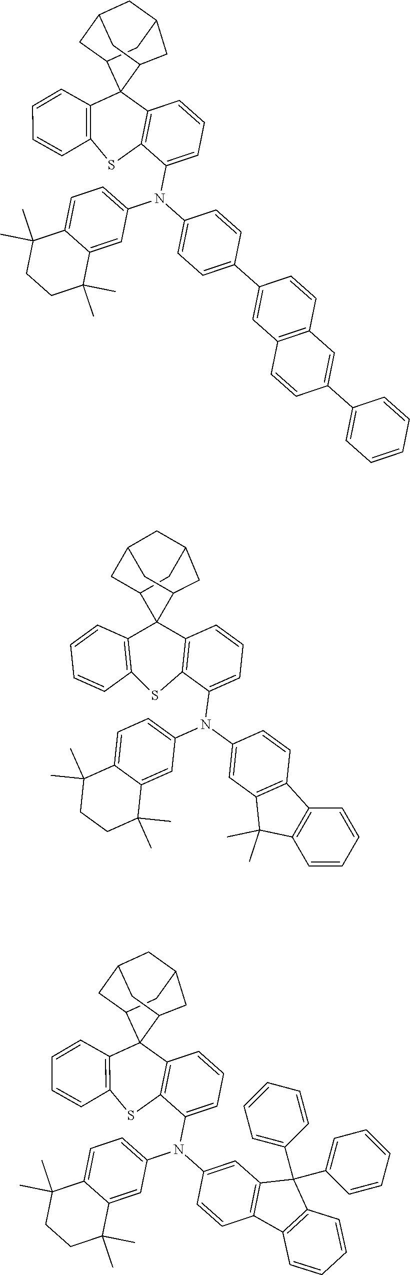 [SMILES: CC1(C)CCC(C)(C)c2cc(N(c3ccc(-c4ccc5cc(-c6ccccc6)ccc5c4)cc3)c3cccc4c3Sc3ccccc3C43C4CC5CC(C4)CC3C5)ccc21 ; CC1(C)CCC(C)(C)c2cc(N(c3ccc4c(c3)C(C)(C)c3ccccc3-4)c3cccc4c3Sc3ccccc3C43C4CC5CC(C4)CC3C5)ccc21 ; CC1(C)CCC(C)(C)c2cc(N(c3ccc4c(c3)C(c3ccccc3)(c3ccccc3)c3ccccc3-4)c3cccc4c3Sc3ccccc3C43C4CC5CC(C4)CC3C5)ccc21]

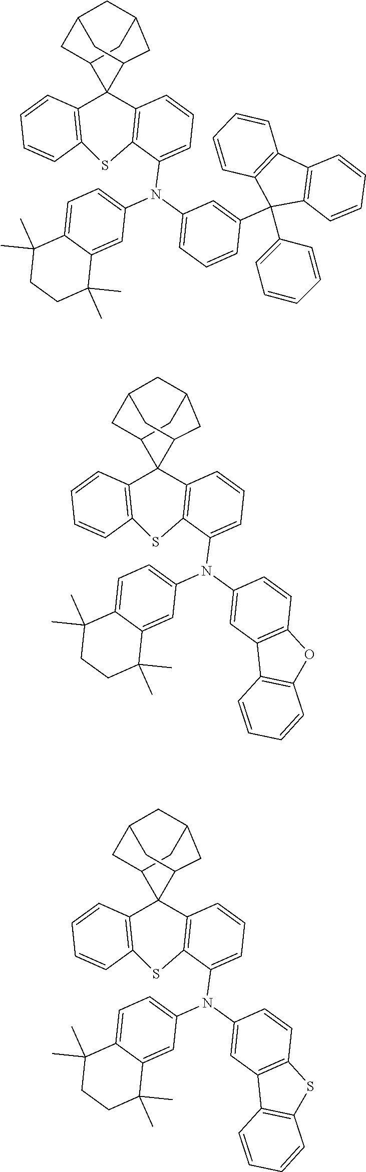 [SMILES: CC1(C)CCC(C)(C)c2cc(N(c3cccc(C4(c5ccccc5)c5ccccc5-c5ccccc54)c3)c3cccc4c3Sc3ccccc3C43C4CC5CC(C4)CC3C5)ccc21 ; CC1(C)CCC(C)(C)c2cc(N(c3ccc4oc5ccccc5c4c3)c3cccc4c3Sc3ccccc3C43C4CC5CC(C4)CC3C5)ccc21 ; CC1(C)CCC(C)(C)c2cc(N(c3ccc4sc5ccccc5c4c3)c3cccc4c3Sc3ccccc3C43C4CC5CC(C4)CC3C5)ccc21]

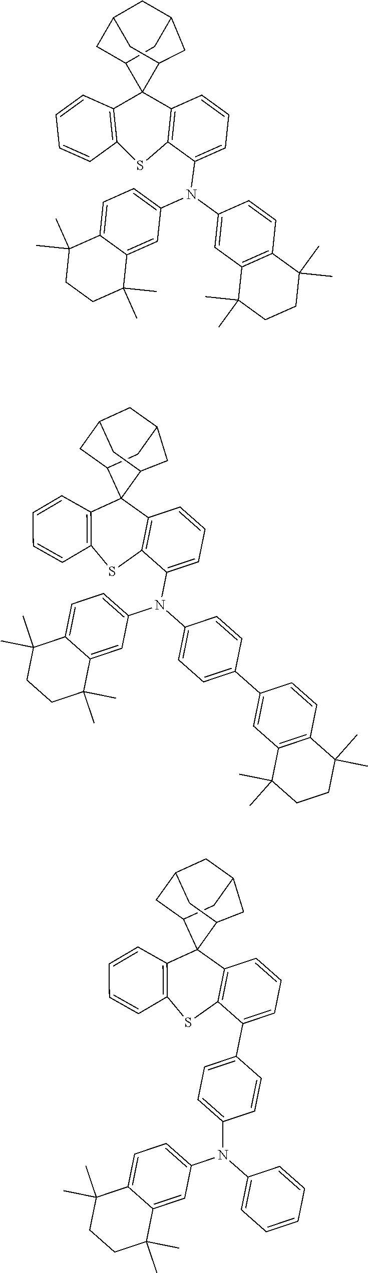

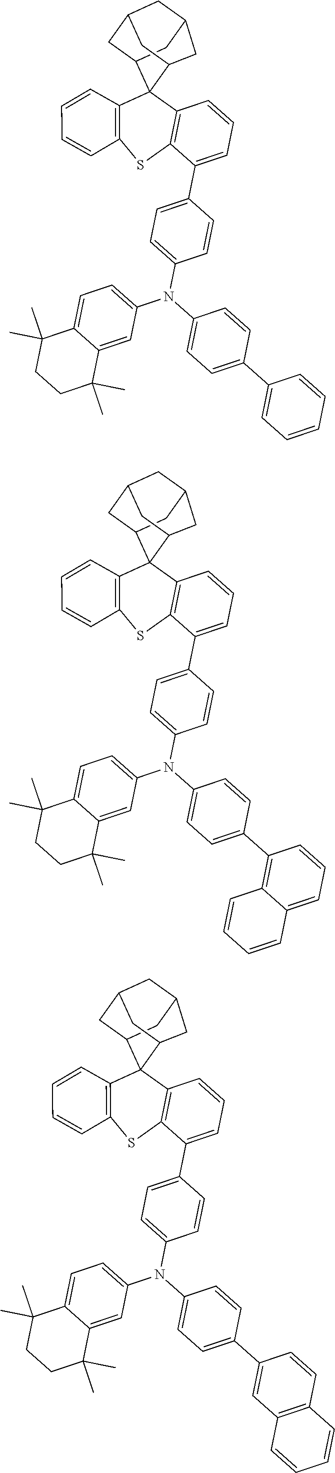 [SMILES: CC1(C)CCC(C)(C)c2cc(N(c3ccc(-c4ccccc4)cc3)c3ccc(-c4cccc5c4Sc4ccccc4C54C5CC6CC(C5)CC4C6)cc3)ccc21 ; CC1(C)CCC(C)(C)c2cc(N(c3ccc(-c4cccc5c4Sc4ccccc4C54C5CC6CC(C5)CC4C6)cc3)c3ccc(-c4cccc5ccccc45)cc3)ccc21 ; CC1(C)CCC(C)(C)c2cc(N(c3ccc(-c4ccc5ccccc5c4)cc3)c3ccc(-c4cccc5c4Sc4ccccc4C54C5CC6CC(C5)CC4C6)cc3)ccc21]

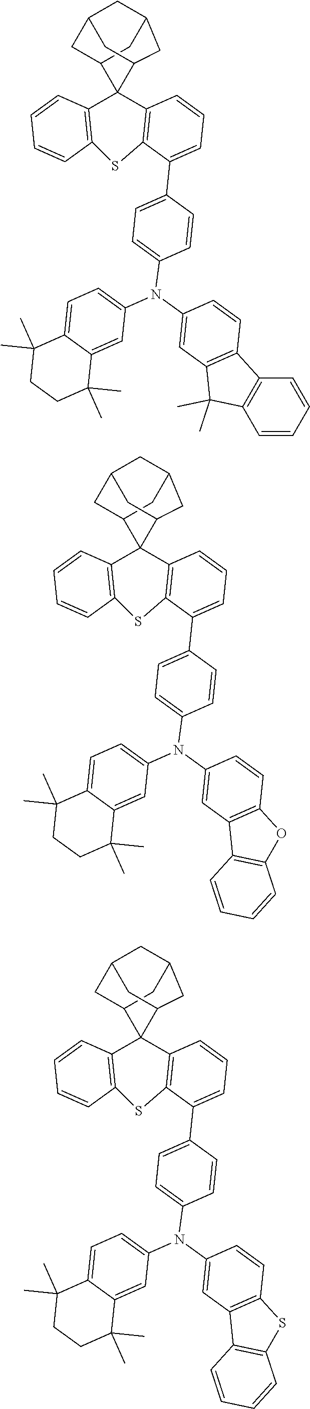 [SMILES: CC1(C)CCC(C)(C)c2cc(N(c3ccc(-c4cccc5c4Sc4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4cccc5c4Sc4ccccc4C54C5CC6CC(C5)CC4C6)cc3)c3ccc4oc5ccccc5c4c3)ccc21 ; CC1(C)CCC(C)(C)c2cc(N(c3ccc(-c4cccc5c4Sc4ccccc4C54C5CC6CC(C5)CC4C6)cc3)c3ccc4sc5ccccc5c4c3)ccc21]

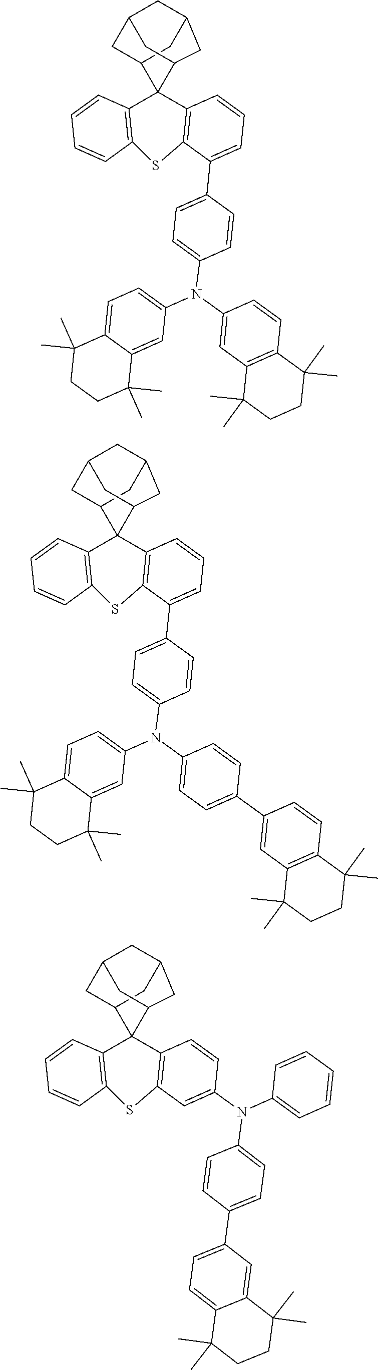

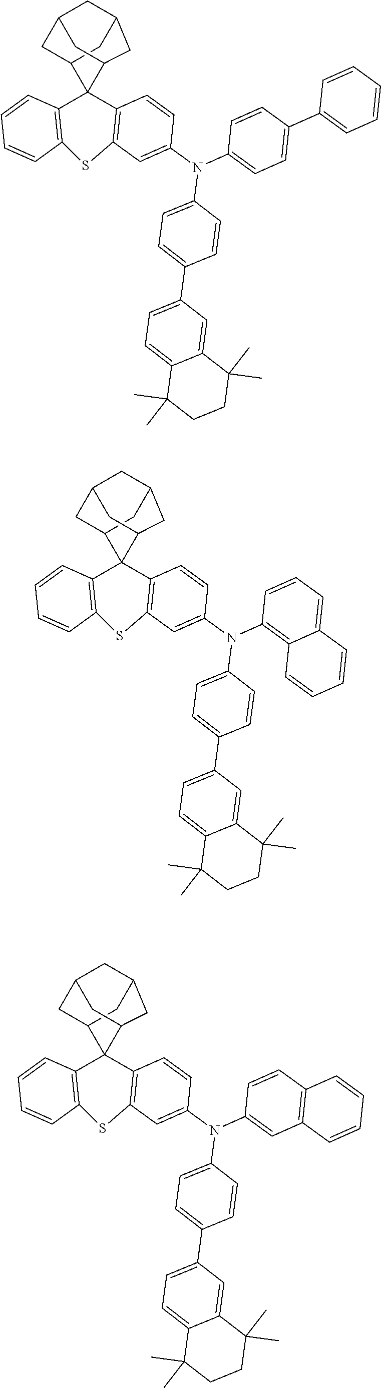 [SMILES: CC1(C)CCC(C)(C)c2cc(-c3ccc(N(c4ccc(-c5ccccc5)cc4)c4ccc5c(c4)Sc4ccccc4C54C5CC6CC(C5)CC4C6)cc3)ccc21 ; CC1(C)CCC(C)(C)c2cc(-c3ccc(N(c4ccc5c(c4)Sc4ccccc4C54C5CC6CC(C5)CC4C6)c4cccc5ccccc45)cc3)ccc21 ; CC1(C)CCC(C)(C)c2cc(-c3ccc(N(c4ccc5c(c4)Sc4ccccc4C54C5CC6CC(C5)CC4C6)c4ccc5ccccc5c4)cc3)ccc21]

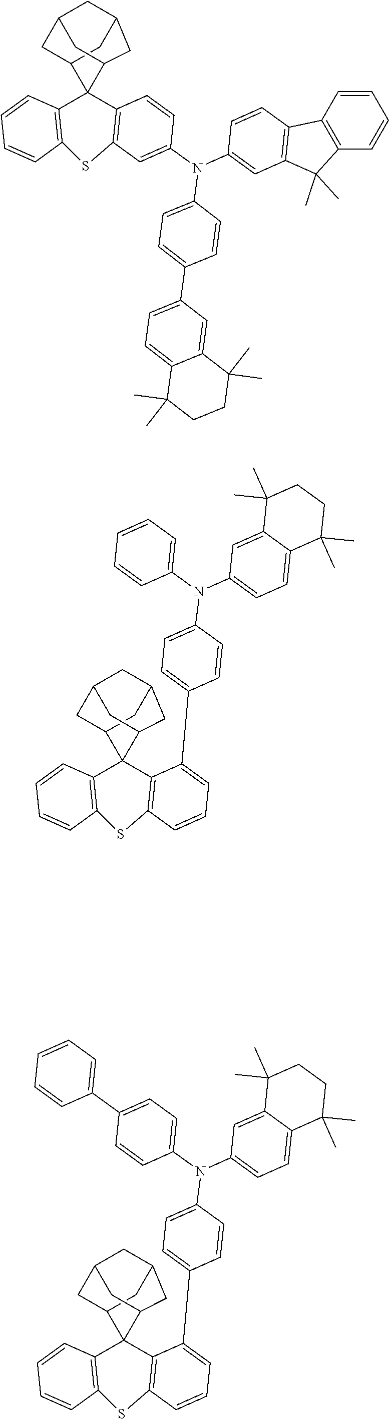 [SMILES: CC1(C)CCC(C)(C)c2cc(-c3ccc(N(c4ccc5c(c4)Sc4ccccc4C54C5CC6CC(C5)CC4C6)c4ccc5c(c4)C(C)(C)c4ccccc4-5)cc3)ccc21 ; CC1(C)CCC(C)(C)c2cc(N(c3ccccc3)c3ccc(-c4cccc5c4C4(c6ccccc6S5)C5CC6CC(C5)CC4C6)cc3)ccc21 ; CC1(C)CCC(C)(C)c2cc(N(c3ccc(-c4ccccc4)cc3)c3ccc(-c4cccc5c4C4(c6ccccc6S5)C5CC6CC(C5)CC4C6)cc3)ccc21]

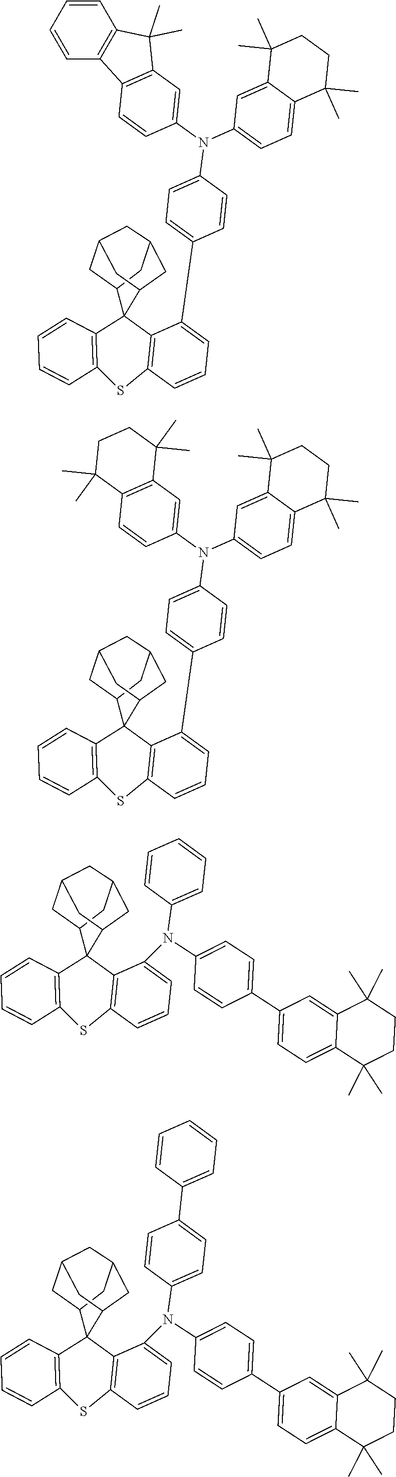

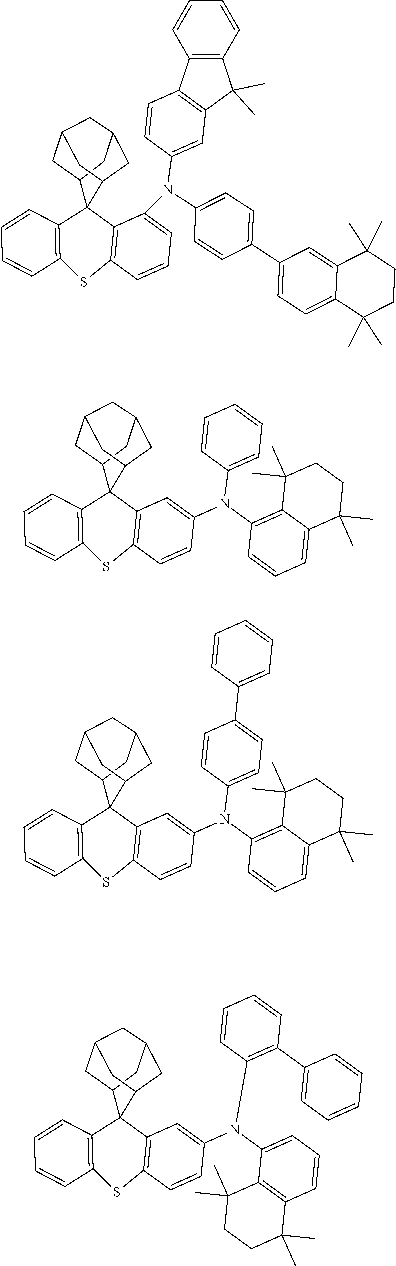 [SMILES: CC1(C)CCC(C)(C)c2cc(-c3ccc(N(c4ccc5c(c4)C(C)(C)c4ccccc4-5)c4cccc5c4C4(c6ccccc6S5)C5CC6CC(C5)CC4C6)cc3)ccc21 ; CC1(C)CCC(C)(C)c2c(N(c3ccccc3)c3ccc4c(c3)C3(c5ccccc5S4)C4CC5CC(C4)CC3C5)cccc21 ; CC1(C)CCC(C)(C)c2c(N(c3ccc(-c4ccccc4)cc3)c3ccc4c(c3)C3(c5ccccc5S4)C4CC5CC(C4)CC3C5)cccc21 ; CC1(C)CCC(C)(C)c2c(N(c3ccc4c(c3)C3(c5ccccc5S4)C4CC5CC(C4)CC3C5)c3ccccc3-c3ccccc3)cccc21]

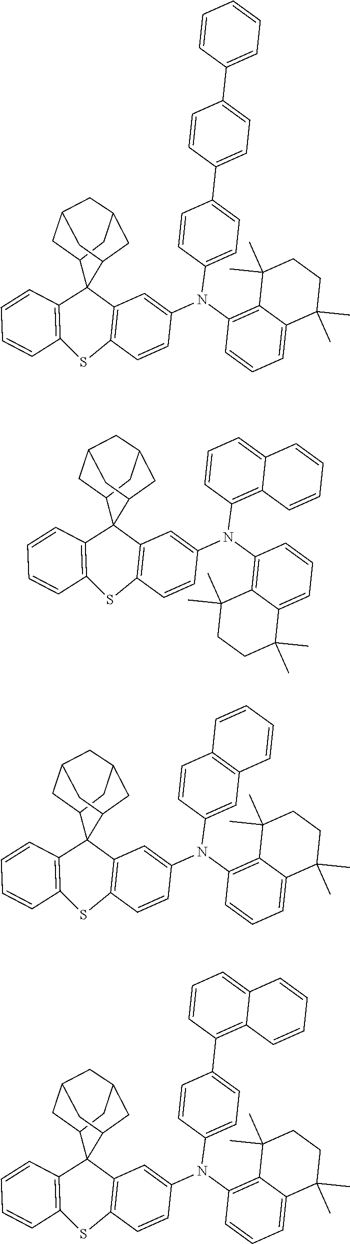 [SMILES: CC1(C)CCC(C)(C)c2c(N(c3ccc(-c4ccc(-c5ccccc5)cc4)cc3)c3ccc4c(c3)C3(c5ccccc5S4)C4CC5CC(C4)CC3C5)cccc21 ; CC1(C)CCC(C)(C)c2c(N(c3ccc4c(c3)C3(c5ccccc5S4)C4CC5CC(C4)CC3C5)c3cccc4ccccc34)cccc21 ; CC1(C)CCC(C)(C)c2c(N(c3ccc4c(c3)C3(c5ccccc5S4)C4CC5CC(C4)CC3C5)c3ccc4ccccc4c3)cccc21 ; CC1(C)CCC(C)(C)c2c(N(c3ccc(-c4cccc5ccccc45)cc3)c3ccc4c(c3)C3(c5ccccc5S4)C4CC5CC(C4)CC3C5)cccc21]

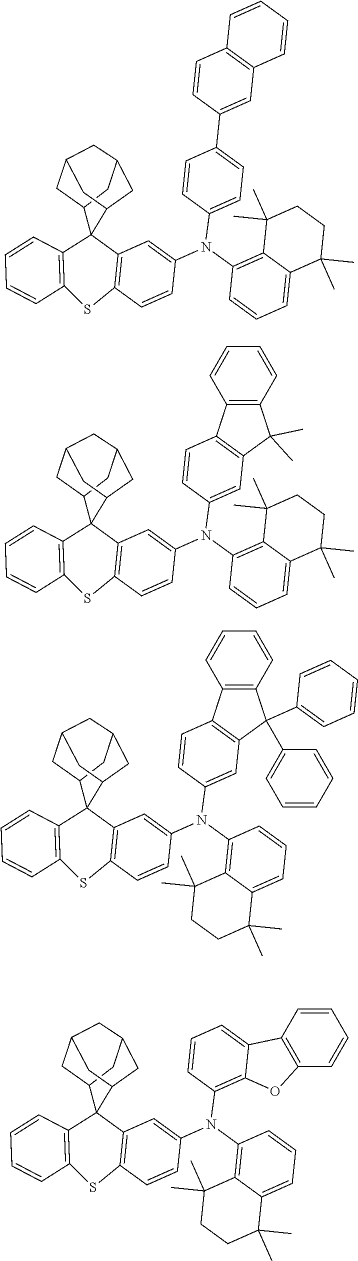

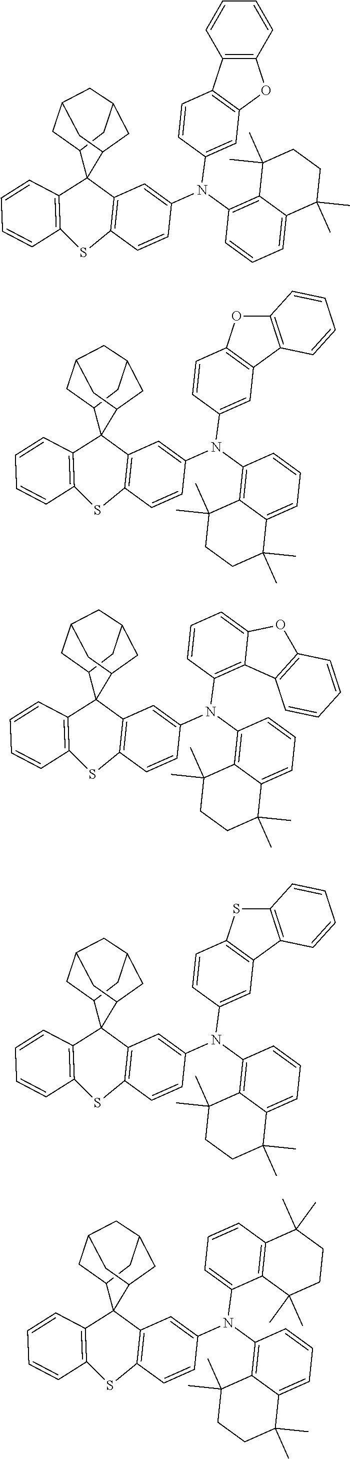

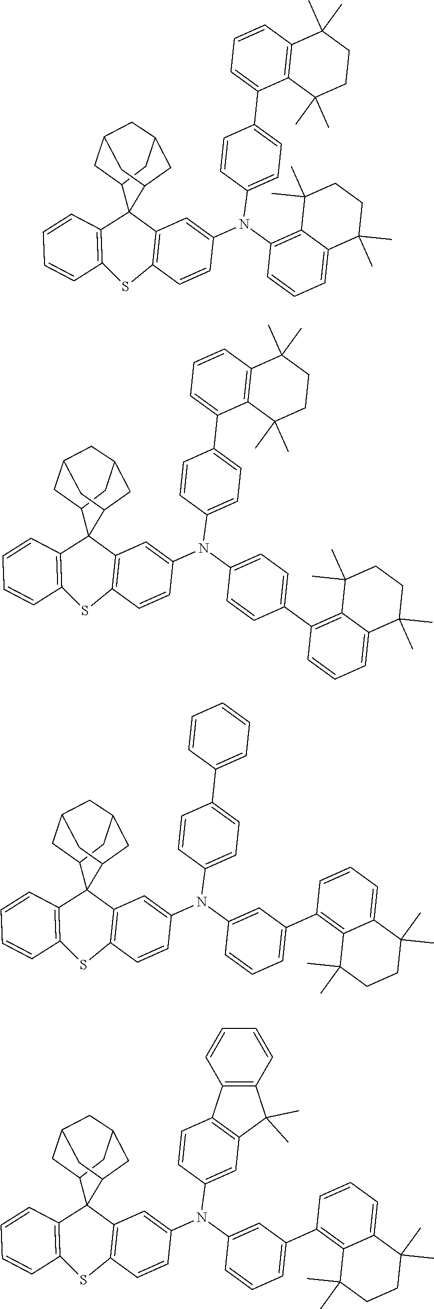

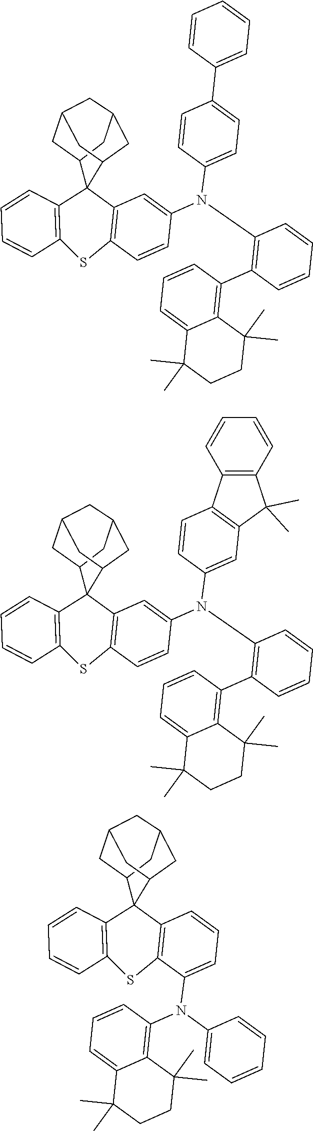

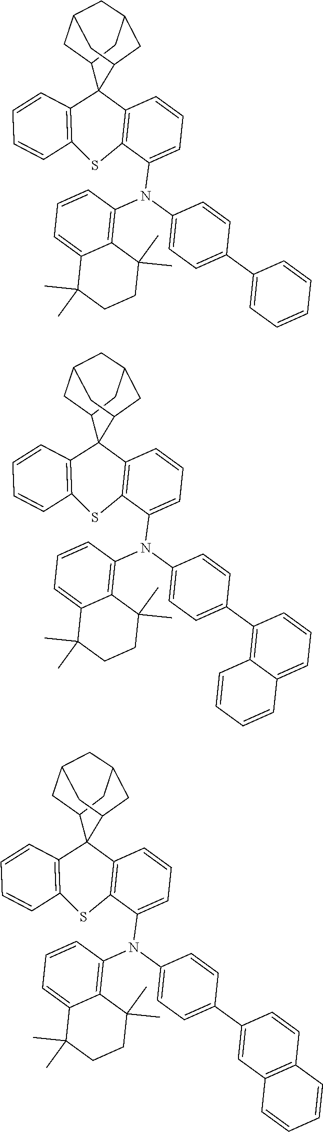

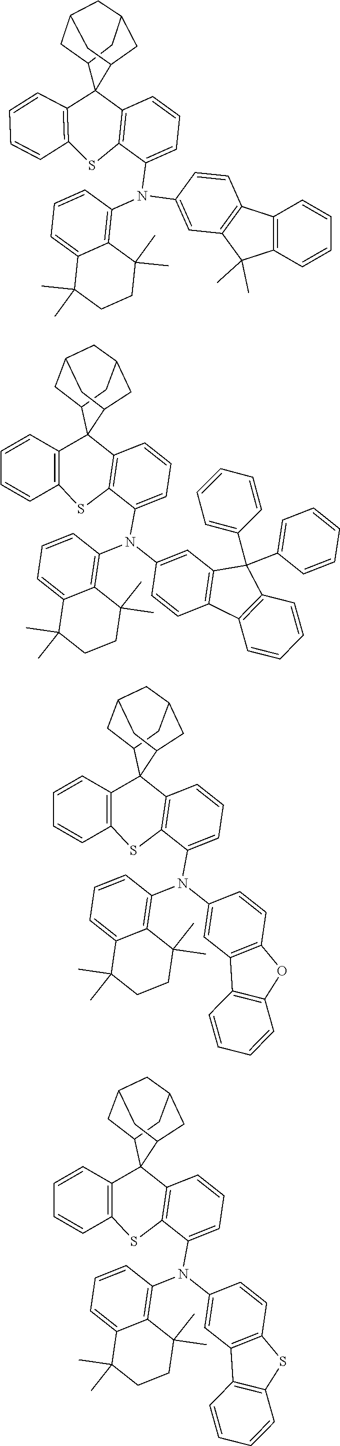

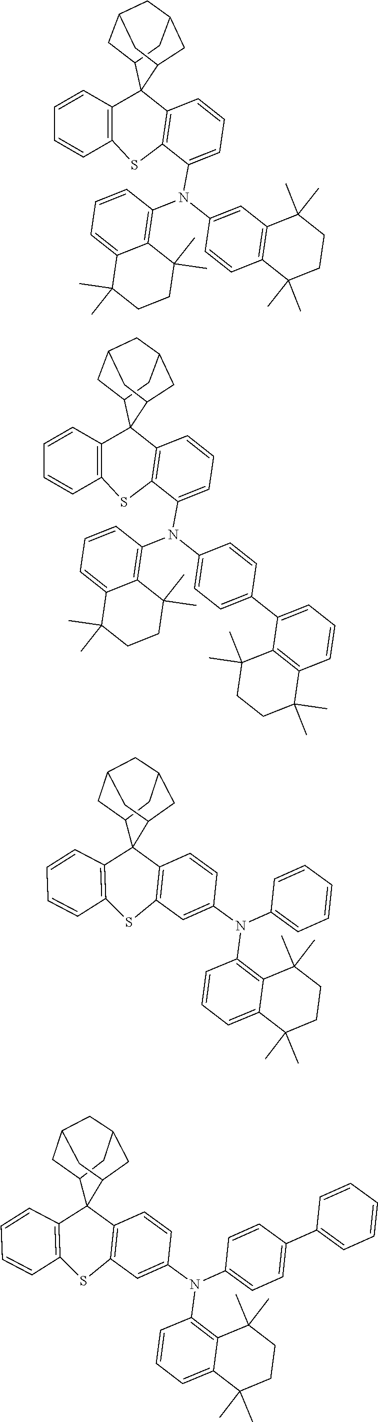

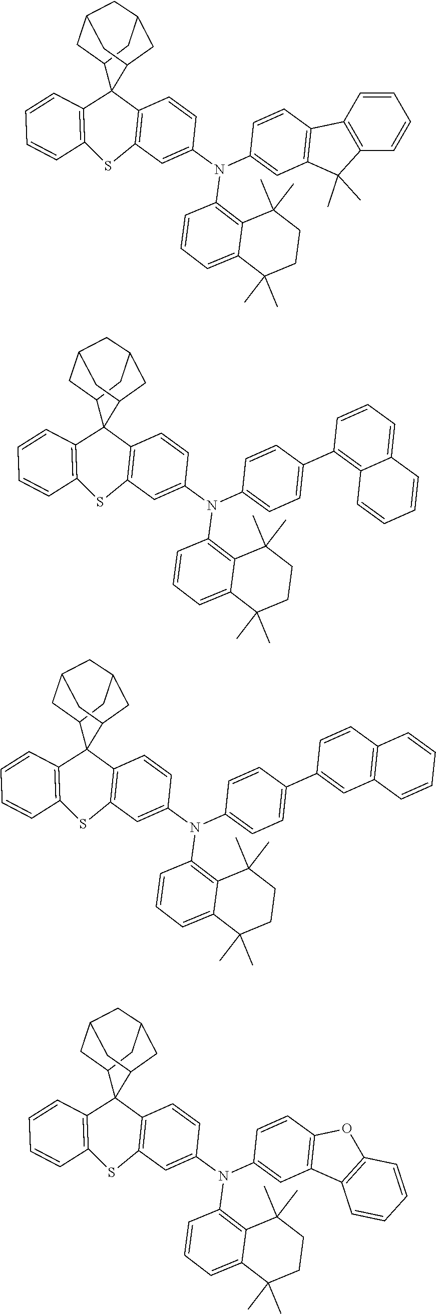 [SMILES: CC1(C)CCC(C)(C)c2c(N(c3ccc4c(c3)Sc3ccccc3C43C4CC5CC(C4)CC3C5)c3ccc4c(c3)C(C)(C)c3ccccc3-4)cccc21 ; CC1(C)CCC(C)(C)c2c(N(c3ccc(-c4cccc5ccccc45)cc3)c3ccc4c(c3)Sc3ccccc3C43C4CC5CC(C4)CC3C5)cccc21 ; CC1(C)CCC(C)(C)c2c(N(c3ccc(-c4ccc5ccccc5c4)cc3)c3ccc4c(c3)Sc3ccccc3C43C4CC5CC(C4)CC3C5)cccc21 ; CC1(C)CCC(C)(C)c2c(N(c3ccc4c(c3)Sc3ccccc3C43C4CC5CC(C4)CC3C5)c3ccc4oc5ccccc5c4c3)cccc21]

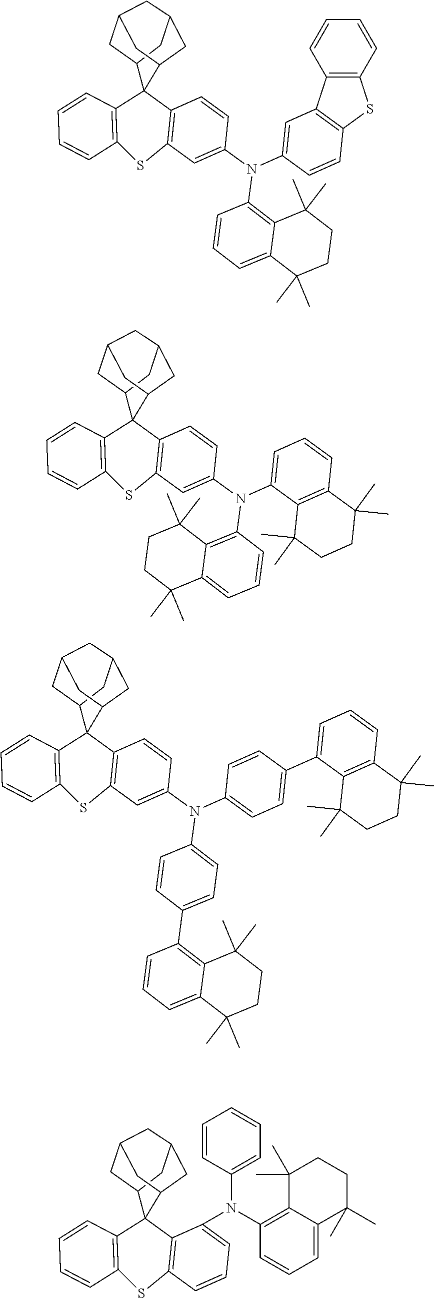 [SMILES: CC1(C)CCC(C)(C)c2c(N(c3ccc4c(c3)Sc3ccccc3C43C4CC5CC(C4)CC3C5)c3ccc4sc5ccccc5c4c3)cccc21 ; CC1(C)CCC(C)(C)c2c(N(c3ccc4c(c3)Sc3ccccc3C43C4CC5CC(C4)CC3C5)c3cccc4c3C(C)(C)CCC4(C)C)cccc21 ; CC1(C)CCC(C)(C)c2c(-c3ccc(N(c4ccc(-c5cccc6c5C(C)(C)CCC6(C)C)cc4)c4ccc5c(c4)Sc4ccccc4C54C5CC6CC(C5)CC4C6)cc3)cccc21 ; CC1(C)CCC(C)(C)c2c(N(c3ccccc3)c3cccc4c3C3(c5ccccc5S4)C4CC5CC(C4)CC3C5)cccc21]

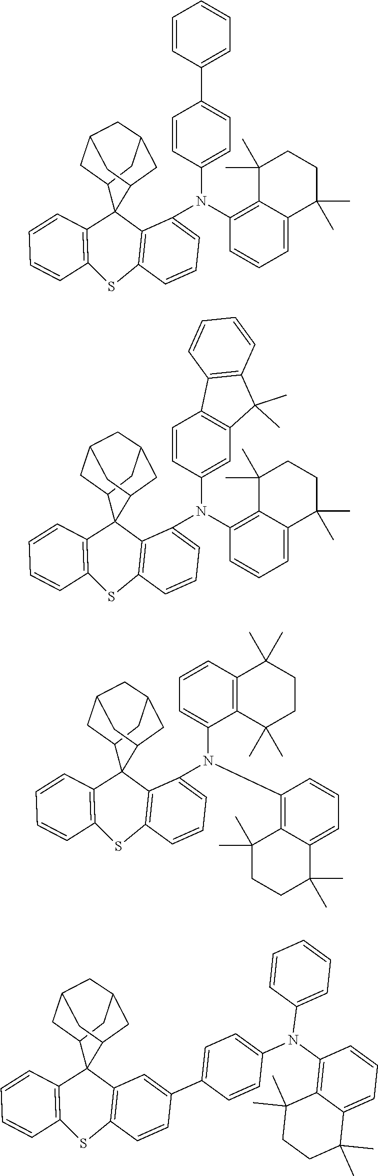

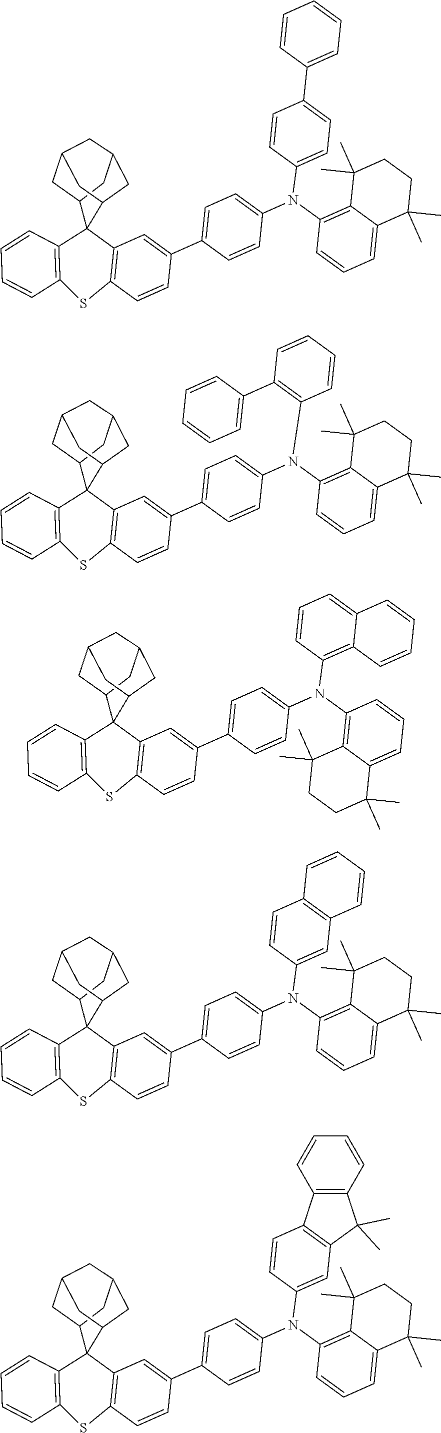

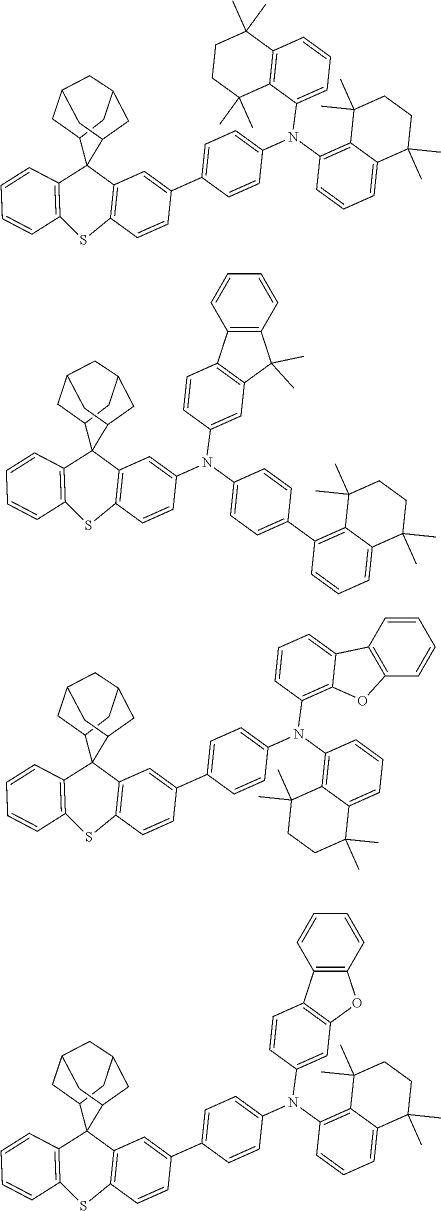

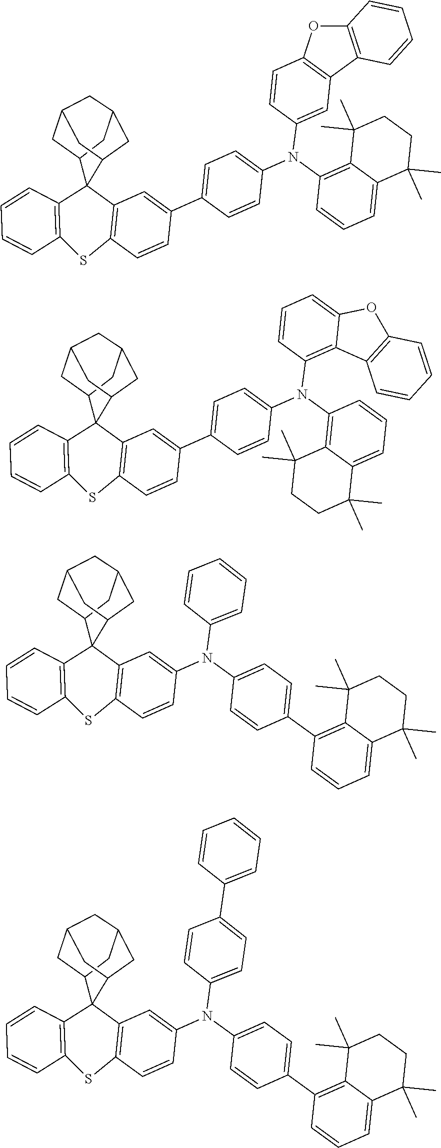 [SMILES: CC1(C)CCC(C)(C)c2c(N(c3ccc(-c4ccc5c(c4)C4(c6ccccc6S5)C5CC6CC(C5)CC4C6)cc3)c3ccc4oc5ccccc5c4c3)cccc21 ; CC1(C)CCC(C)(C)c2c(N(c3ccc(-c4ccc5c(c4)C4(c6ccccc6S5)C5CC6CC(C5)CC4C6)cc3)c3cccc4oc5ccccc5c34)cccc21 ; CC1(C)CCC(C)(C)c2c(-c3ccc(N(c4ccccc4)c4ccc5c(c4)C4(c6ccccc6S5)C5CC6CC(C5)CC4C6)cc3)cccc21 ; CC1(C)CCC(C)(C)c2c(-c3ccc(N(c4ccc(-c5ccccc5)cc4)c4ccc5c(c4)C4(c6ccccc6S5)C5CC6CC(C5)CC4C6)cc3)cccc21]

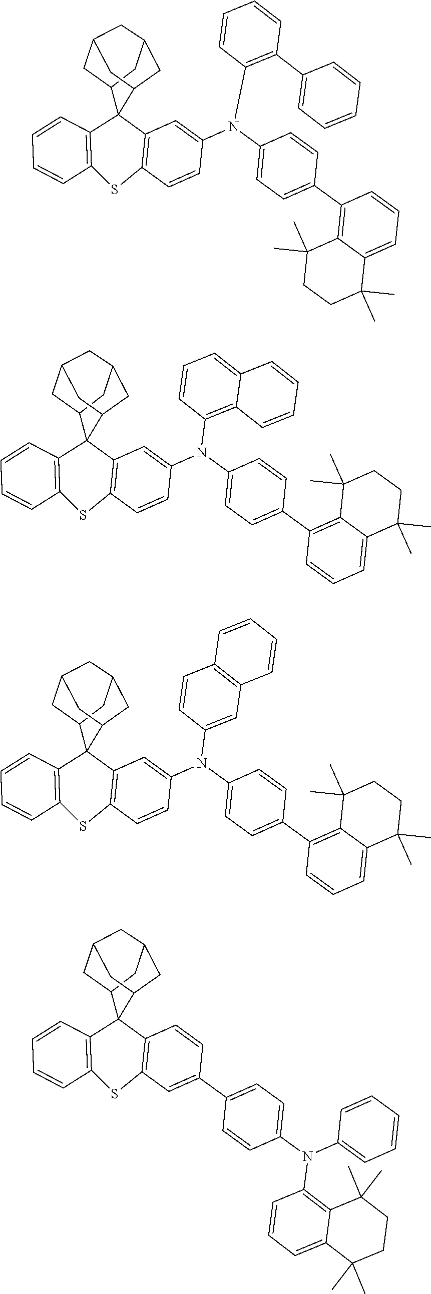 [SMILES: CC1(C)CCC(C)(C)c2c(-c3ccc(N(c4ccc5c(c4)C4(c6ccccc6S5)C5CC6CC(C5)CC4C6)c4ccccc4-c4ccccc4)cc3)cccc21 ; CC1(C)CCC(C)(C)c2c(-c3ccc(N(c4ccc5c(c4)C4(c6ccccc6S5)C5CC6CC(C5)CC4C6)c4cccc5ccccc45)cc3)cccc21 ; CC1(C)CCC(C)(C)c2c(-c3ccc(N(c4ccc5c(c4)C4(c6ccccc6S5)C5CC6CC(C5)CC4C6)c4ccc5ccccc5c4)cc3)cccc21 ; CC1(C)CCC(C)(C)c2c(N(c3ccccc3)c3ccc(-c4ccc5c(c4)Sc4ccccc4C54C5CC6CC(C5)CC4C6)cc3)cccc21]

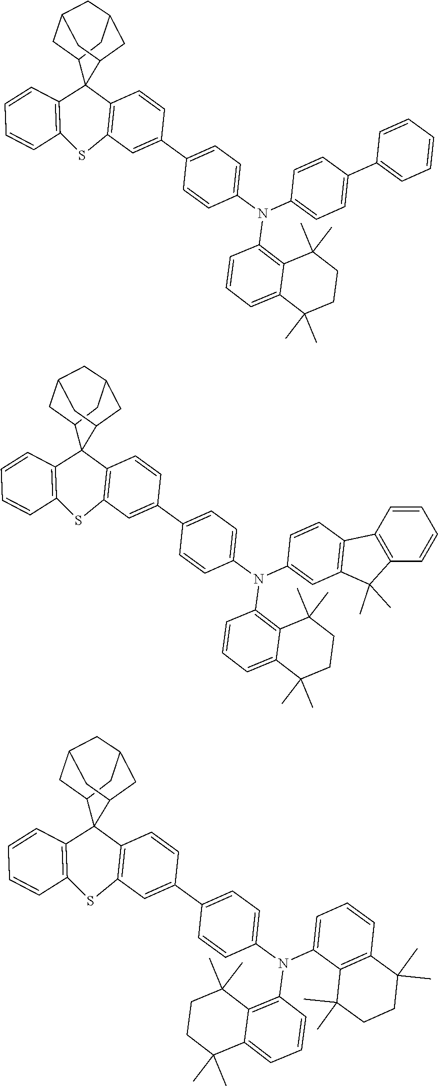 [SMILES: CC1(C)CCC(C)(C)c2c(N(c3ccc(-c4ccccc4)cc3)c3ccc(-c4ccc5c(c4)Sc4ccccc4C54C5CC6CC(C5)CC4C6)cc3)cccc21 ; CC1(C)CCC(C)(C)c2c(N(c3ccc(-c4ccc5c(c4)Sc4ccccc4C54C5CC6CC(C5)CC4C6)cc3)c3ccc4c(c3)C(C)(C)c3ccccc3-4)cccc21 ; CC1(C)CCC(C)(C)c2c(N(c3ccc(-c4ccc5c(c4)Sc4ccccc4C54C5CC6CC(C5)CC4C6)cc3)c3cccc4c3C(C)(C)CCC4(C)C)cccc21]

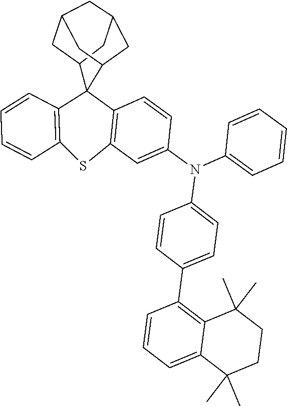 [SMILES: CC1(C)CCC(C)(C)c2c(-c3ccc(N(c4ccccc4)c4ccc5c(c4)Sc4ccccc4C54C5CC6CC(C5)CC4C6)cc3)cccc21]

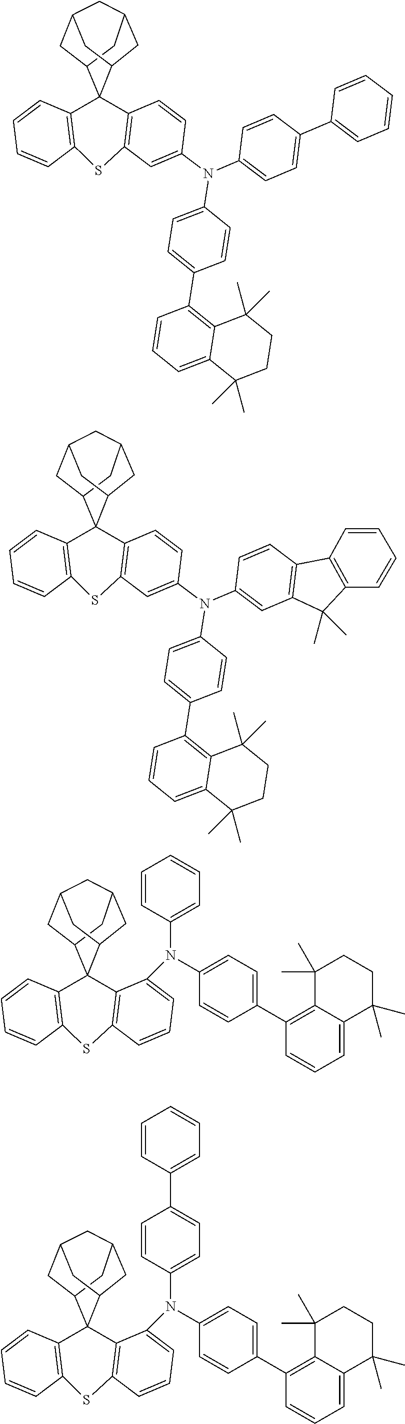

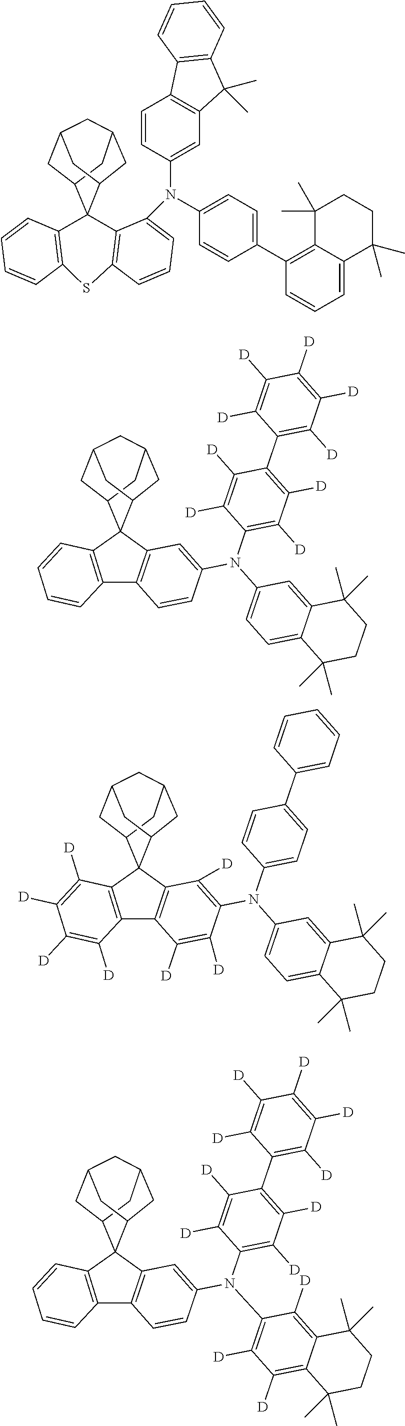

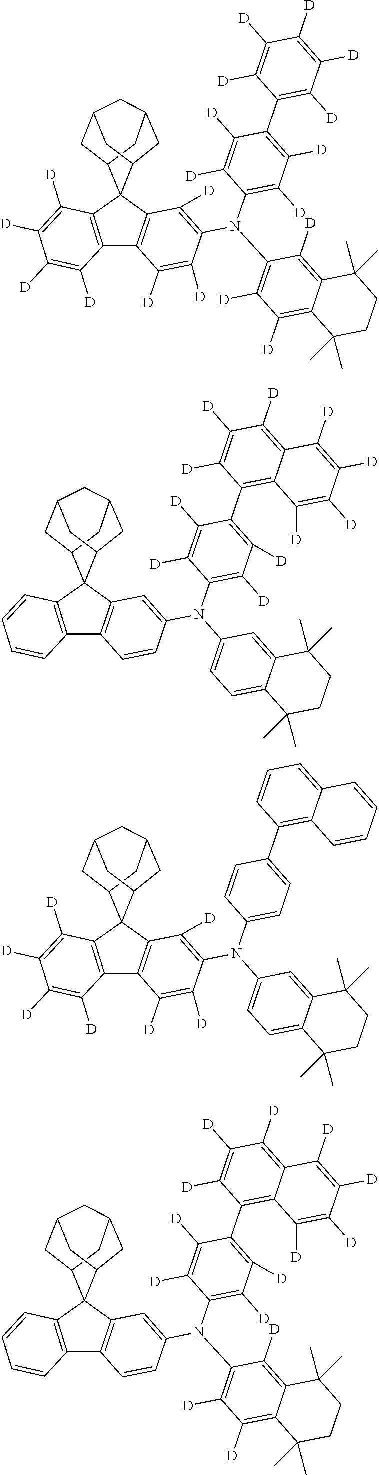

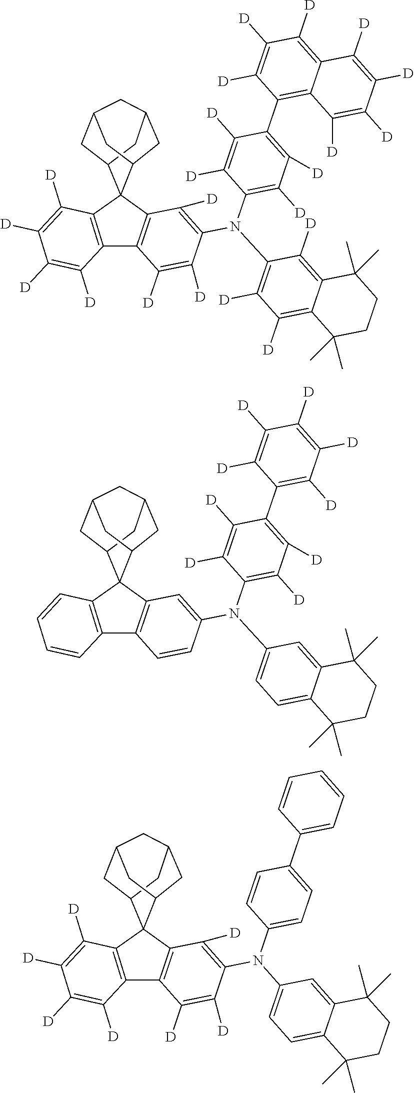

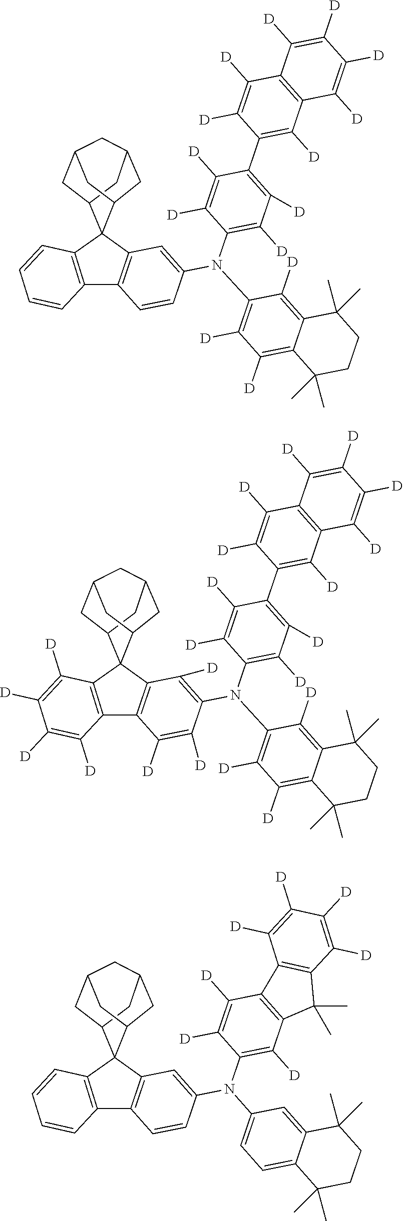

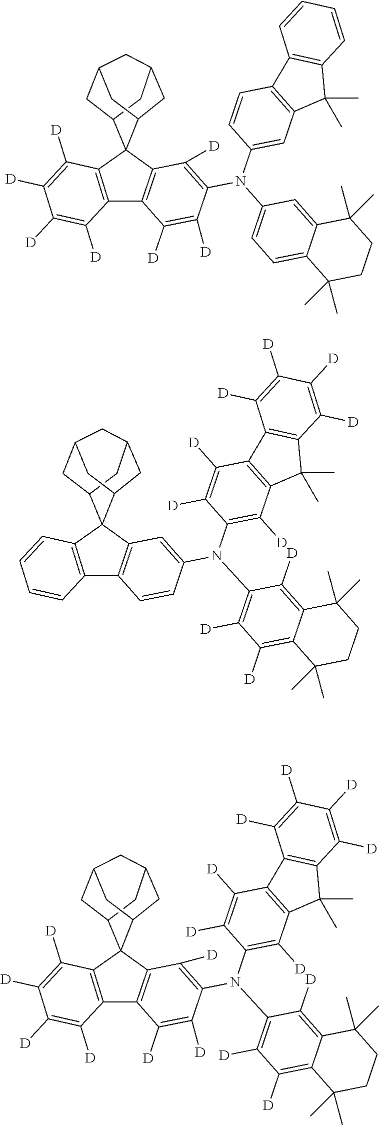

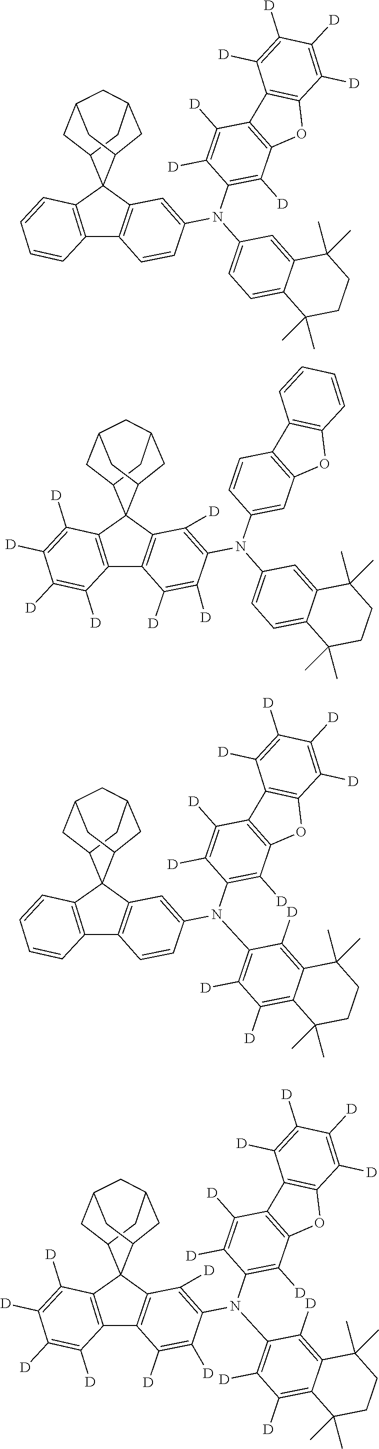

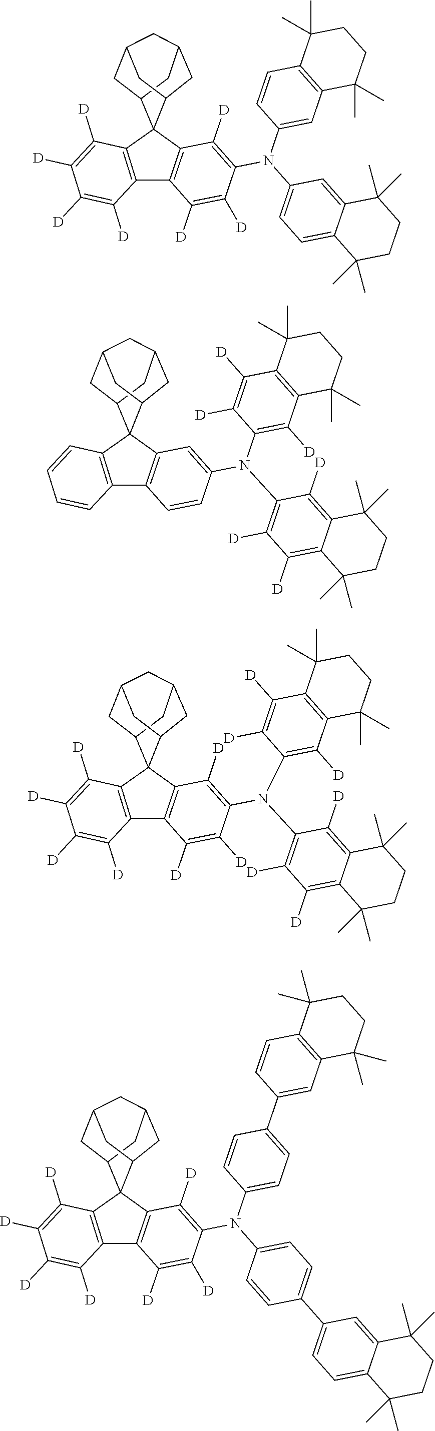

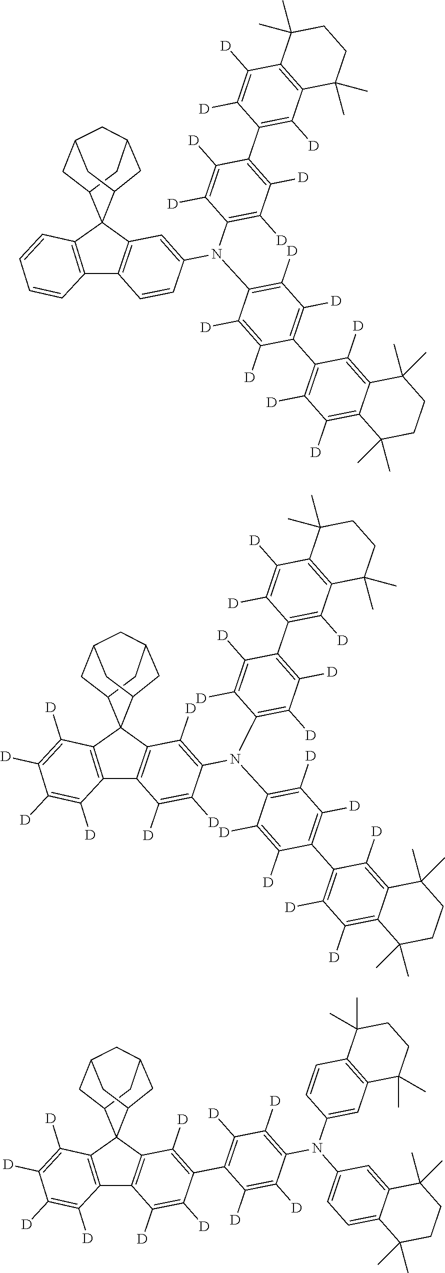

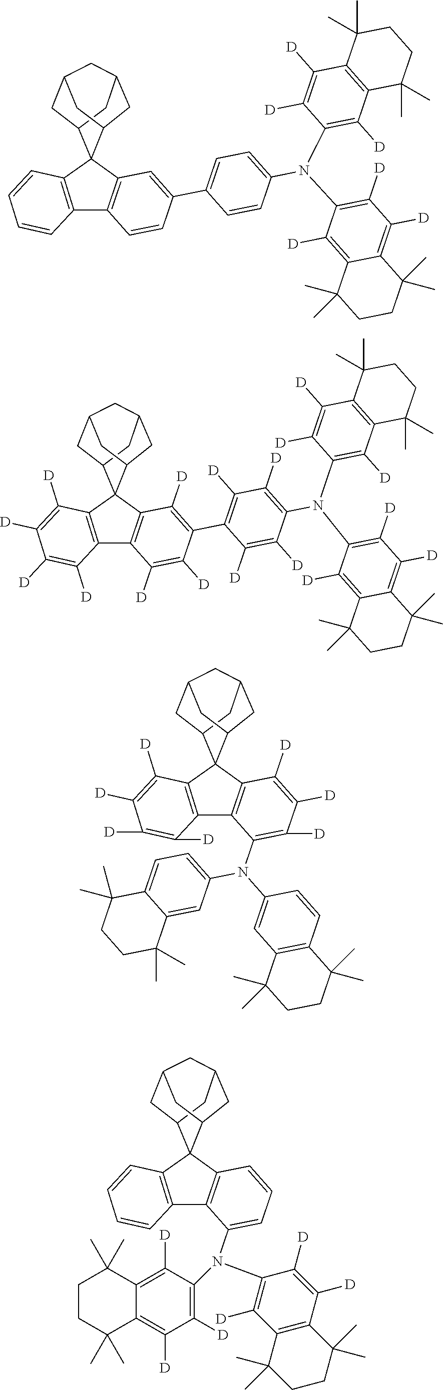

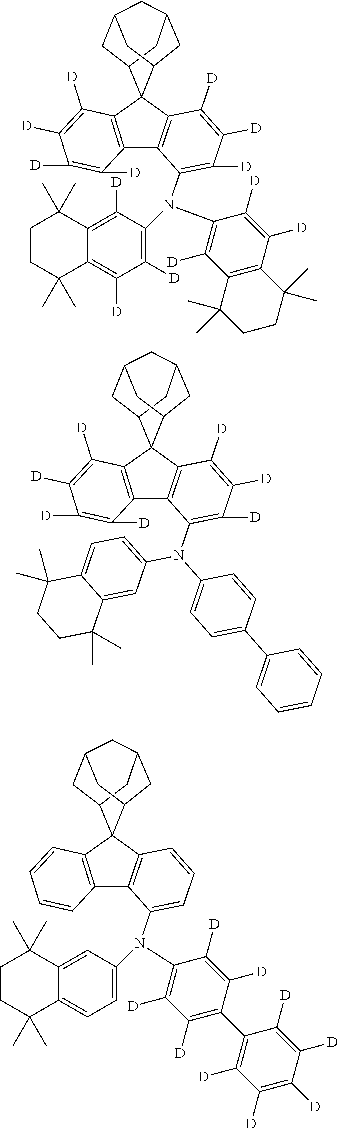

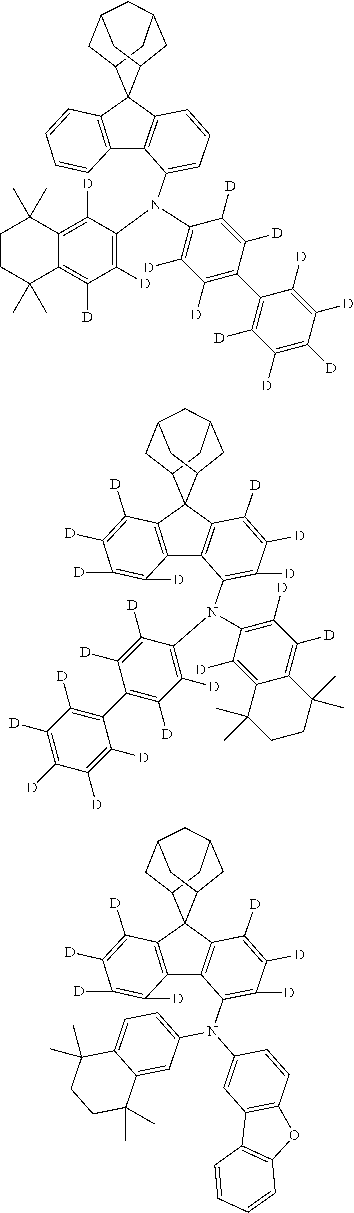

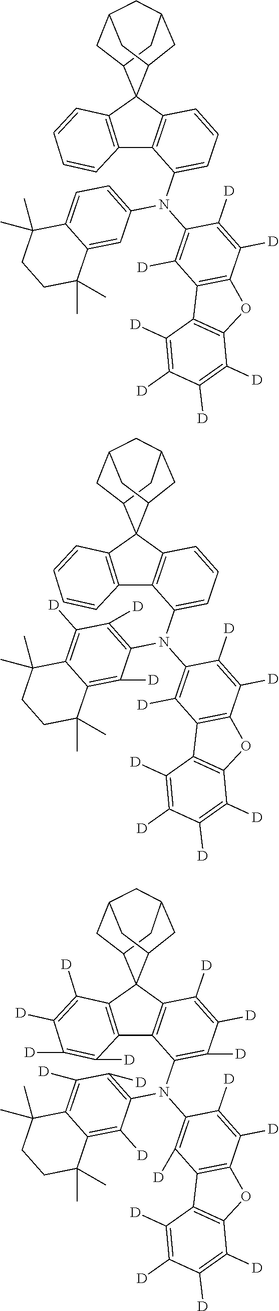 [SMILES: [2H]c1c([2H])c([2H])c2c(oc3c([2H])c([2H])c(N(c4ccc5c(c4)C(C)(C)CCC5(C)C)c4cccc5c4-c4ccccc4C54C5CC6CC(C5)CC4C6)c([2H])c32)c1[2H] ; [2H]c1c([2H])c2c(c([2H])c1N(c1cccc3c1-c1ccccc1C31C3CC4CC(C3)CC1C4)c1c([2H])c([2H])c3oc4c([2H])c([2H])c([2H])c([2H])c4c3c1[2H])C(C)(C)CCC2(C)C ; [2H]c1c([2H])c([2H])c2c(c1[2H])-c1c(N(c3c([2H])c([2H])c4c(c3[2H])C(C)(C)CCC4(C)C)c3c([2H])c([2H])c4oc5c([2H])c([2H])c([2H])c([2H])c5c4c3[2H])c([2H])c([2H])c([2H])c1C21C2CC3CC(C2)CC1C3]

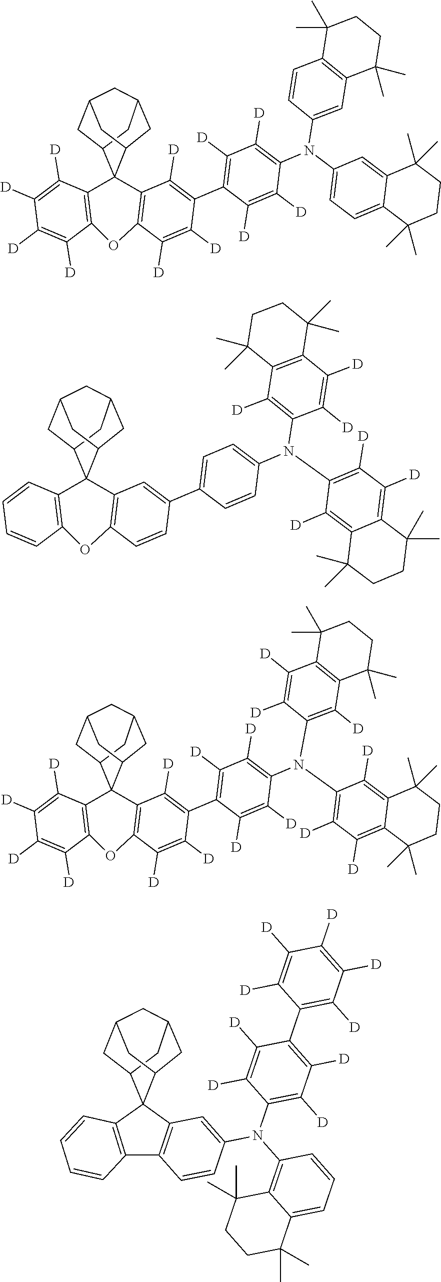

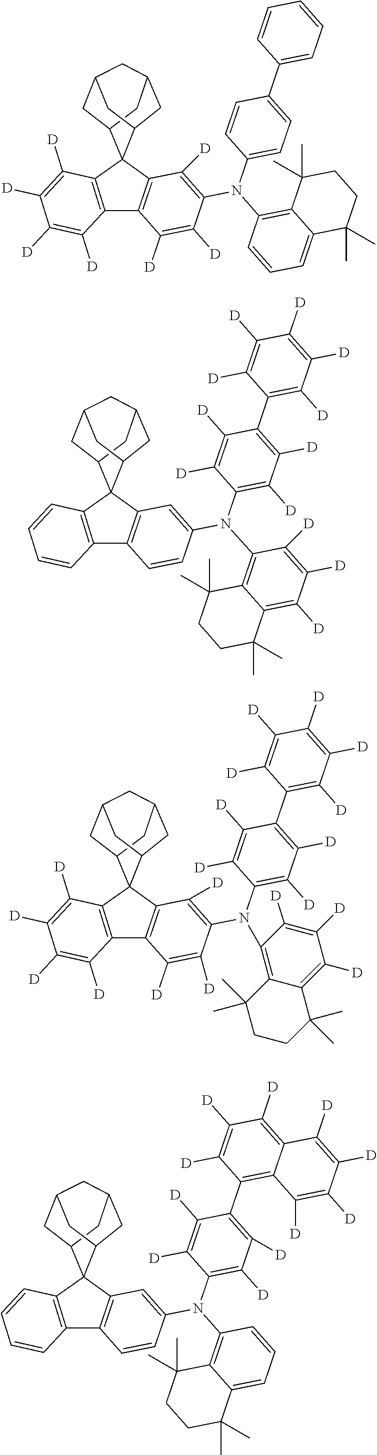

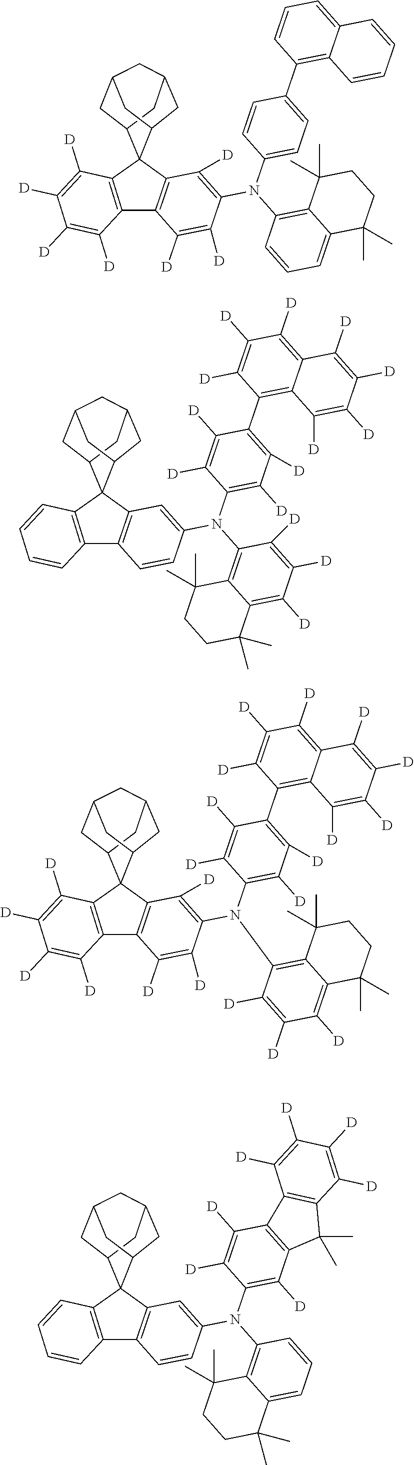

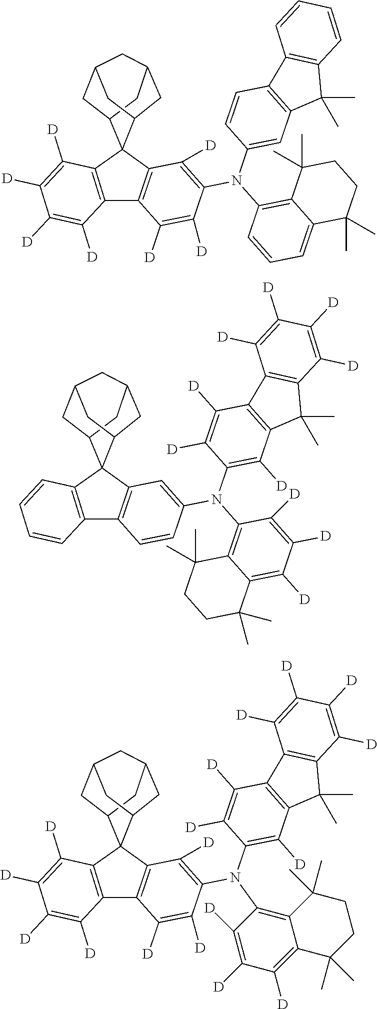

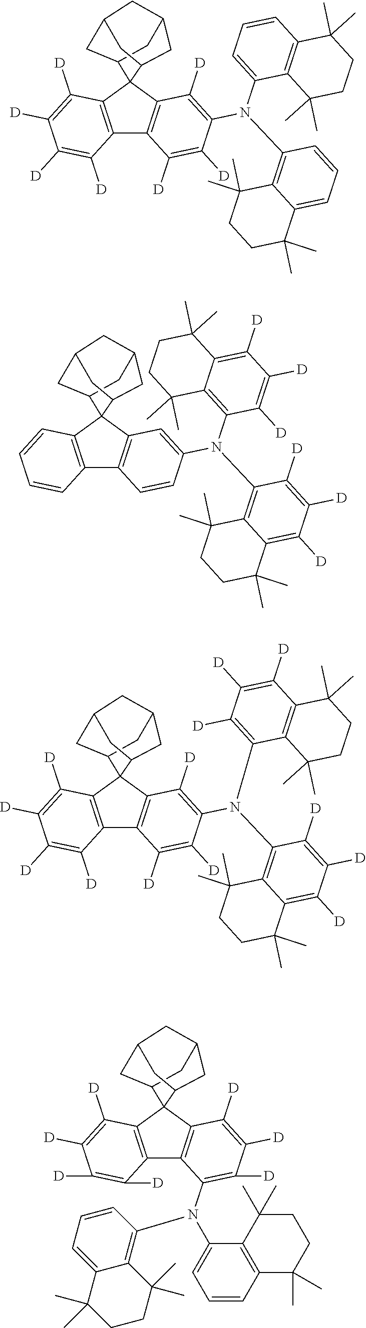

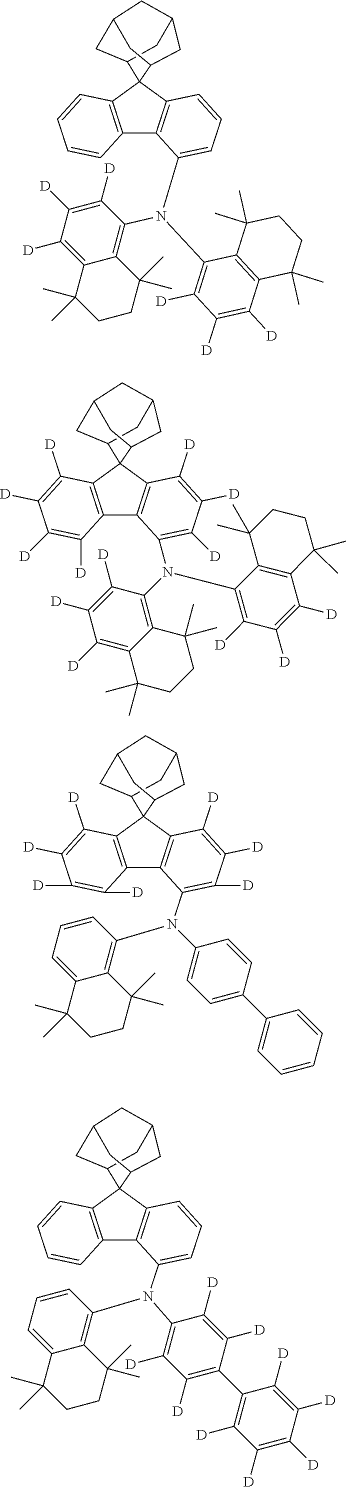

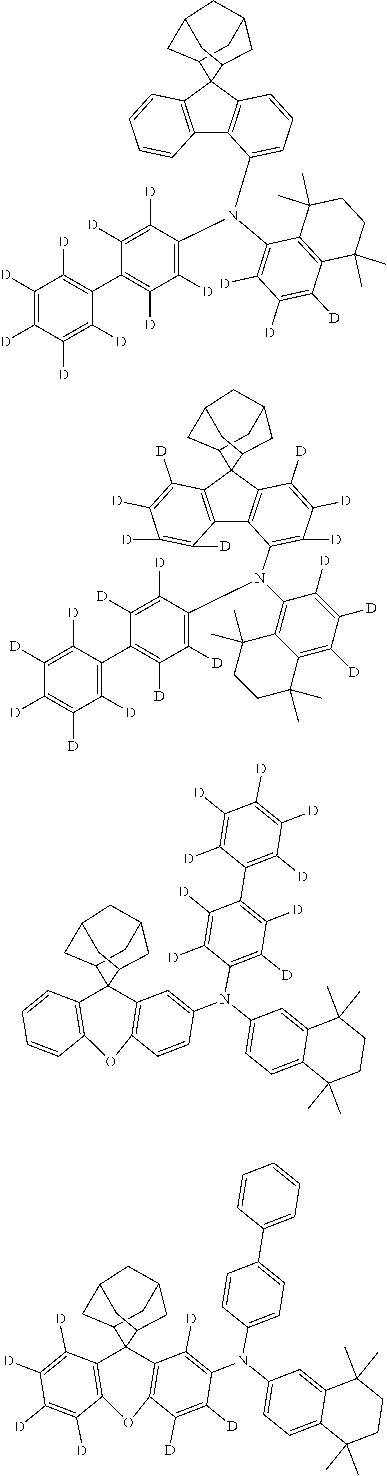

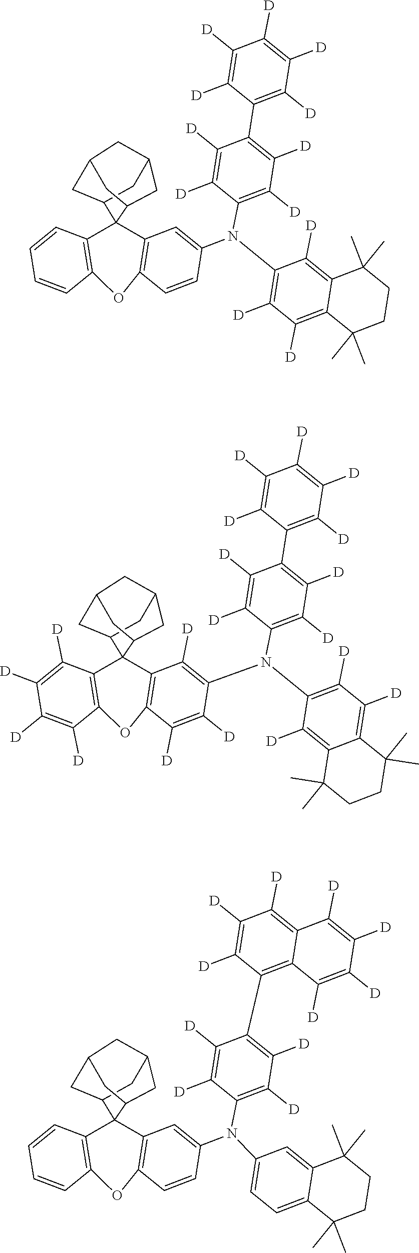

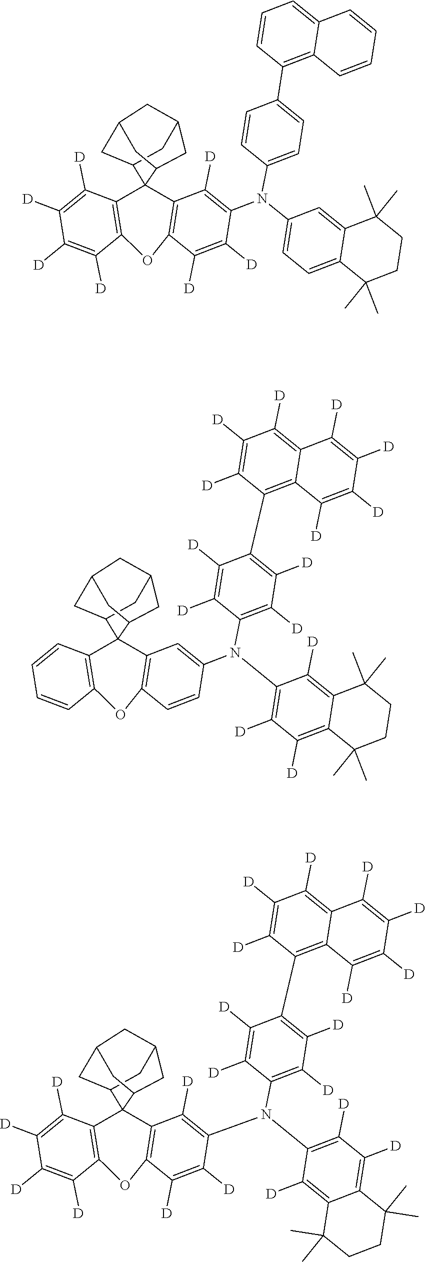

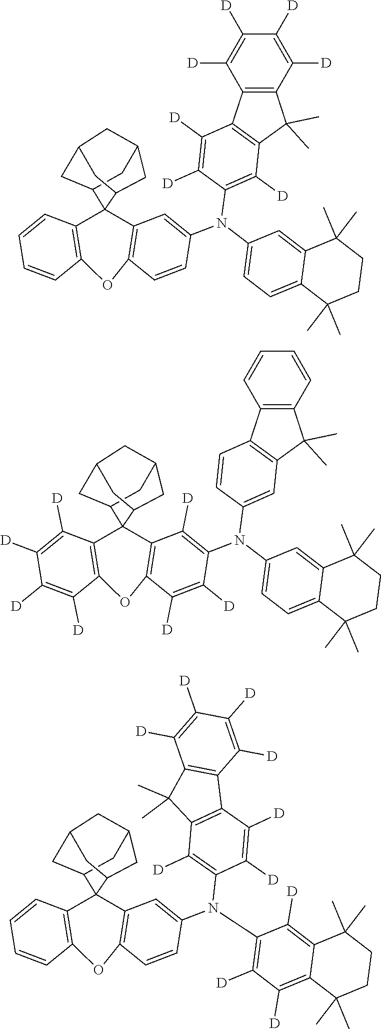

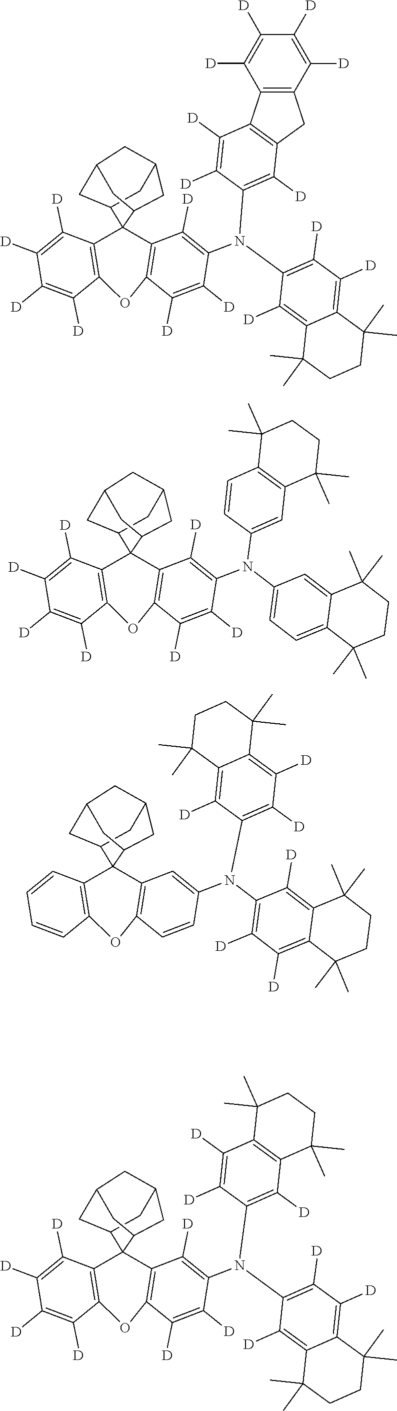

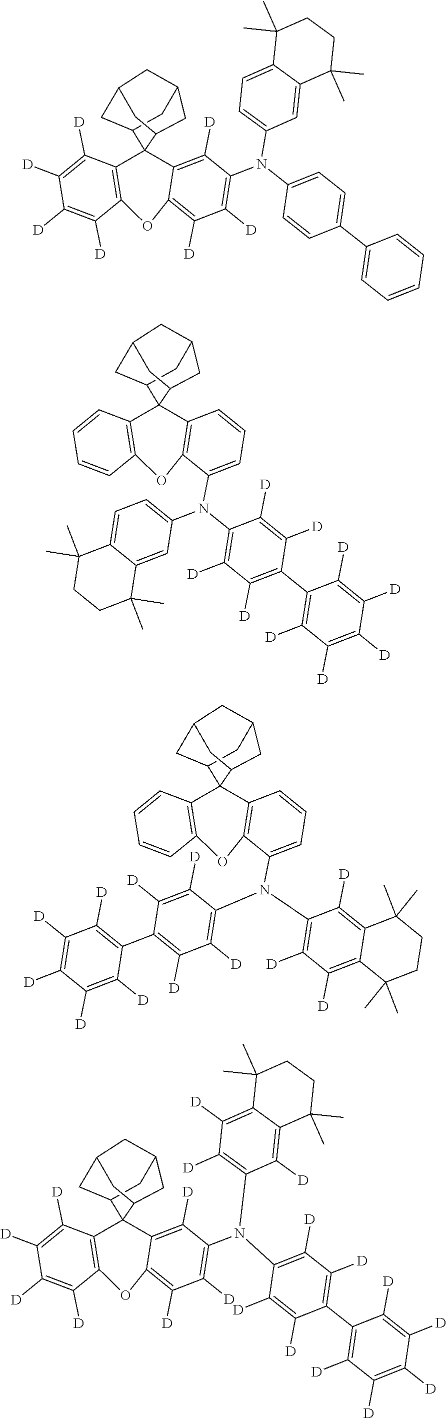

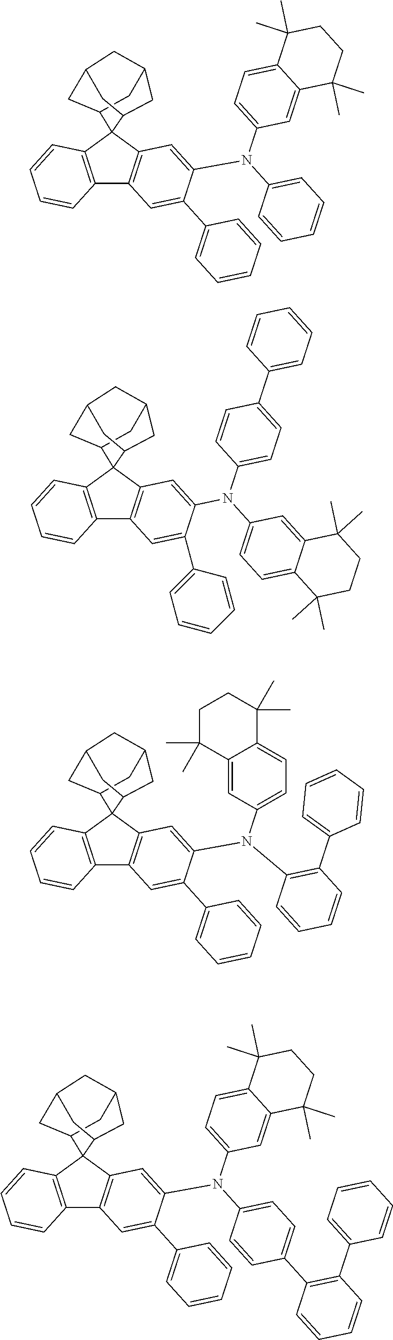

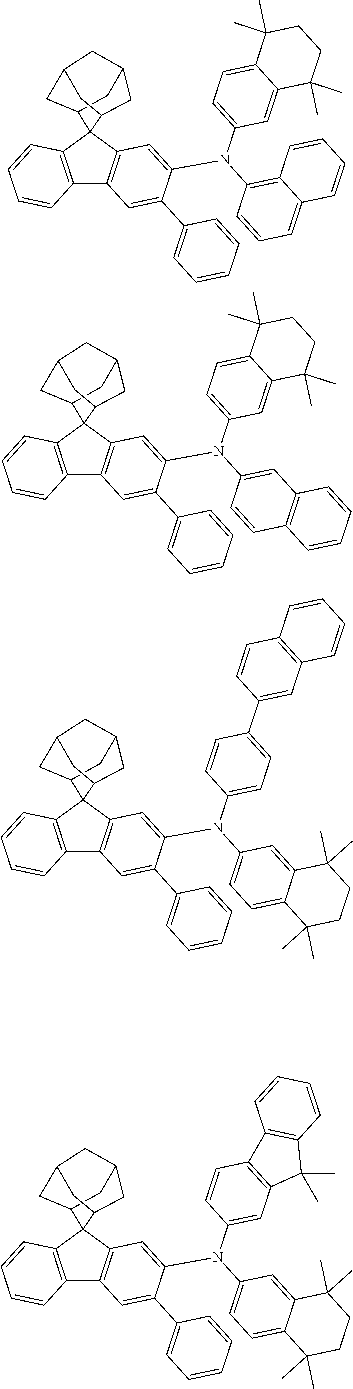

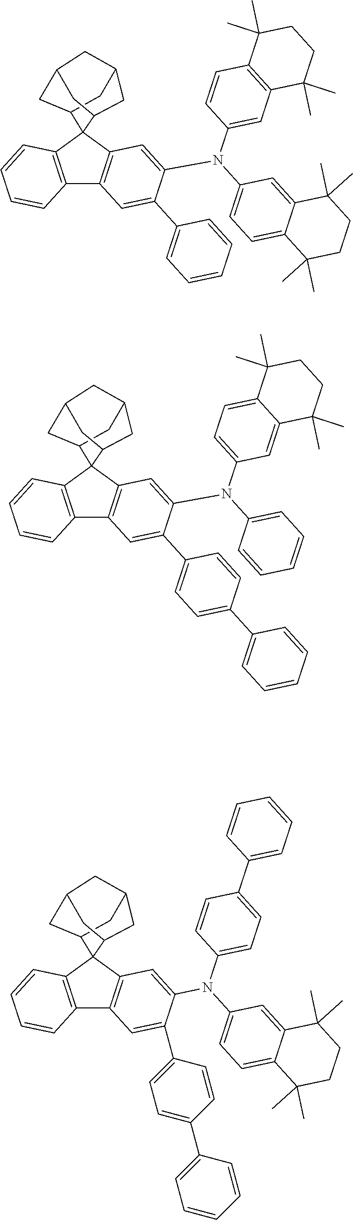 [SMILES: CC1(C)CCC(C)(C)c2cc(N(c3ccc4c(c3)C(C)(C)CCC4(C)C)c3cc4c(cc3-c3ccccc3)-c3ccccc3C43C4CC5CC(C4)CC3C5)ccc21 ; CC1(C)CCC(C)(C)c2cc(N(c3ccccc3)c3cc4c(cc3-c3ccc(-c5ccccc5)cc3)-c3ccccc3C43C4CC5CC(C4)CC3C5)ccc21 ; CC1(C)CCC(C)(C)c2cc(N(c3ccc(-c4ccccc4)cc3)c3cc4c(cc3-c3ccc(-c5ccccc5)cc3)-c3ccccc3C43C4CC5CC(C4)CC3C5)ccc21]

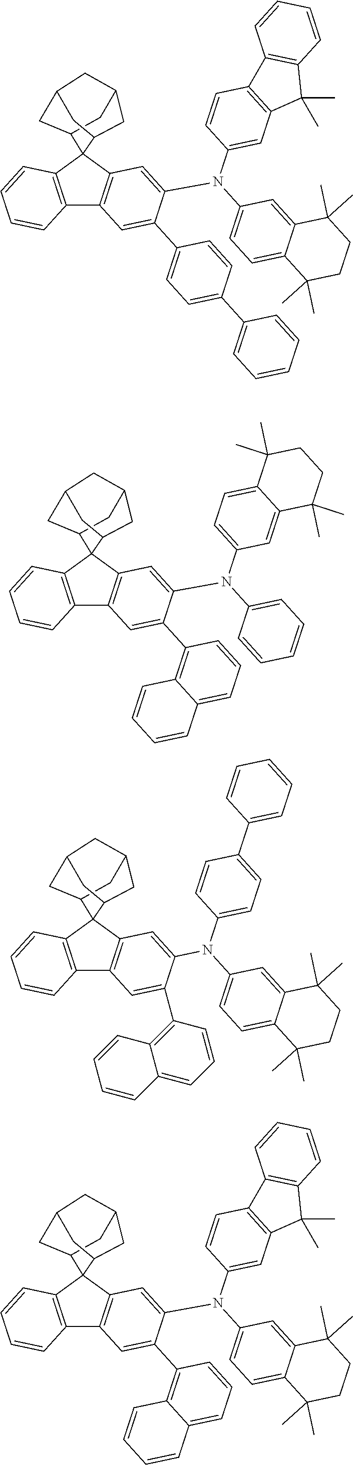

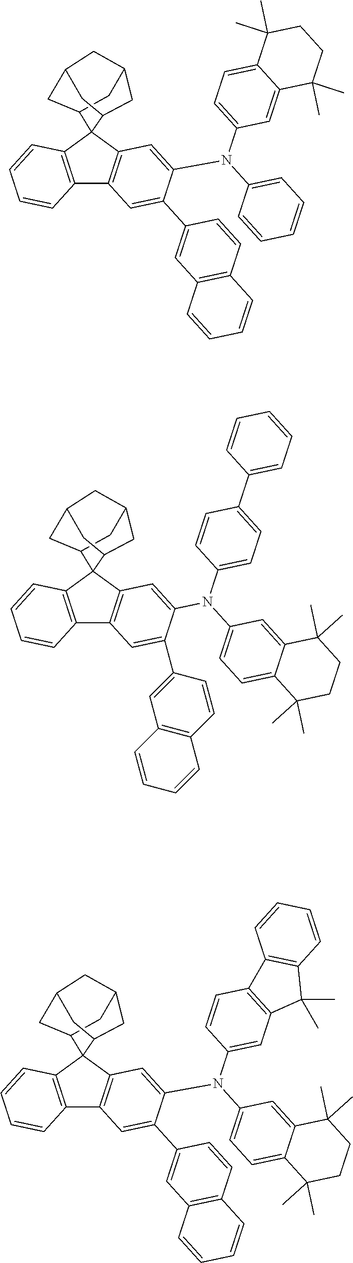 [SMILES: CC1(C)CCC(C)(C)c2cc(N(c3ccccc3)c3cc4c(cc3-c3ccc5ccccc5c3)-c3ccccc3C43C4CC5CC(C4)CC3C5)ccc21 ; CC1(C)CCC(C)(C)c2cc(N(c3ccc(-c4ccccc4)cc3)c3cc4c(cc3-c3ccc5ccccc5c3)-c3ccccc3C43C4CC5CC(C4)CC3C5)ccc21 ; CC1(C)CCC(C)(C)c2cc(N(c3ccc4c(c3)C(C)(C)c3ccccc3-4)c3cc4c(cc3-c3ccc5ccccc5c3)-c3ccccc3C43C4CC5CC(C4)CC3C5)ccc21]

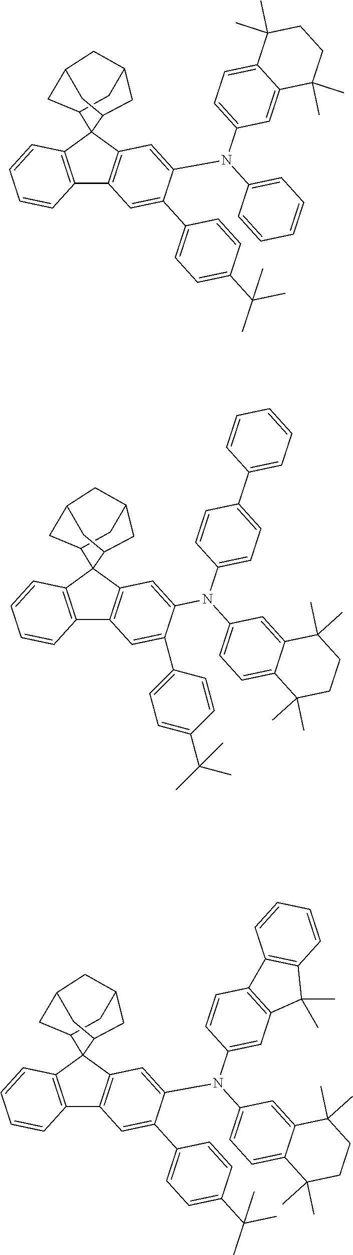 [SMILES: CC(C)(C)c1ccc(-c2cc3c(cc2N(c2ccccc2)c2ccc4c(c2)C(C)(C)CCC4(C)C)C2(c4ccccc4-3)C3CC4CC(C3)CC2C4)cc1 ; CC(C)(C)c1ccc(-c2cc3c(cc2N(c2ccc(-c4ccccc4)cc2)c2ccc4c(c2)C(C)(C)CCC4(C)C)C2(c4ccccc4-3)C3CC4CC(C3)CC2C4)cc1 ; CC(C)(C)c1ccc(-c2cc3c(cc2N(c2ccc4c(c2)C(C)(C)CCC4(C)C)c2ccc4c(c2)C(C)(C)c2ccccc2-4)C2(c4ccccc4-3)C3CC4CC(C3)CC2C4)cc1]

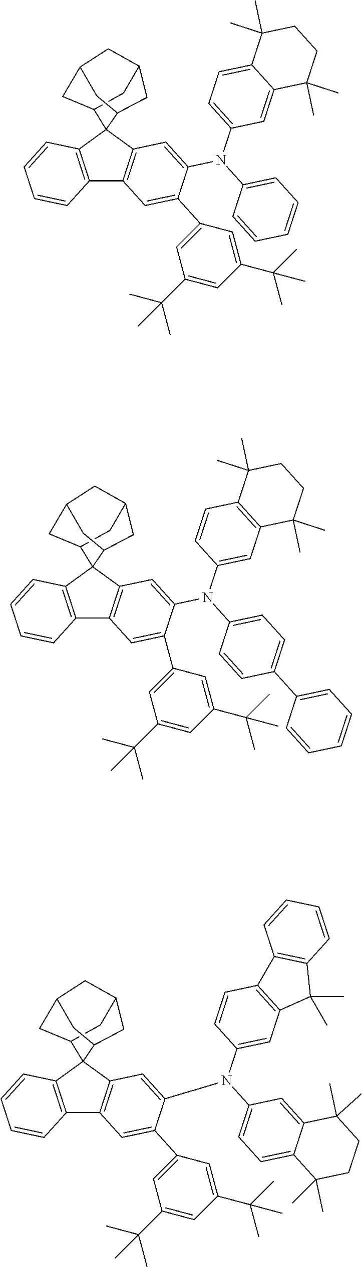 [SMILES: CC(C)(C)c1cc(-c2cc3c(cc2N(c2ccccc2)c2ccc4c(c2)C(C)(C)CCC4(C)C)C2(c4ccccc4-3)C3CC4CC(C3)CC2C4)cc(C(C)(C)C)c1 ; CC(C)(C)c1cc(-c2cc3c(cc2N(c2ccc(-c4ccccc4)cc2)c2ccc4c(c2)C(C)(C)CCC4(C)C)C2(c4ccccc4-3)C3CC4CC(C3)CC2C4)cc(C(C)(C)C)c1 ; CC(C)(C)c1cc(-c2cc3c(cc2N(c2ccc4c(c2)C(C)(C)CCC4(C)C)c2ccc4c(c2)C(C)(C)c2ccccc2-4)C2(c4ccccc4-3)C3CC4CC(C3)CC2C4)cc(C(C)(C)C)c1]

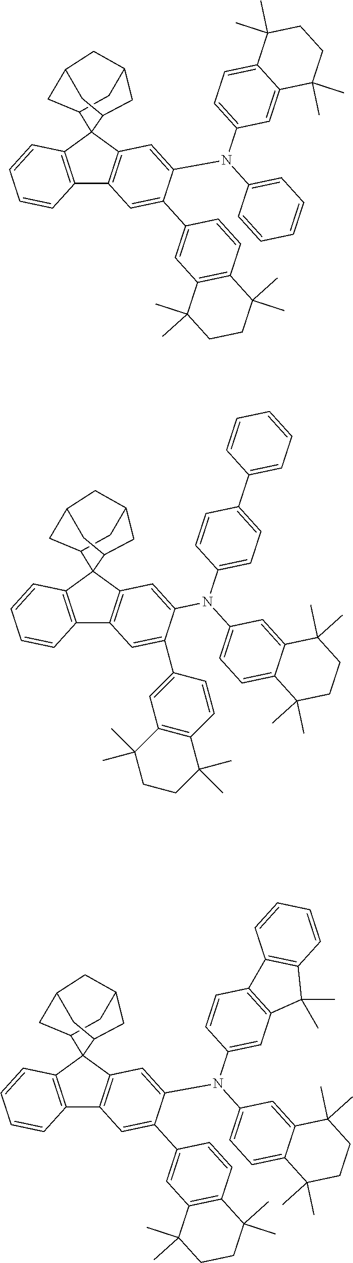 [SMILES: CC1(C)CCC(C)(C)c2cc(-c3cc4c(cc3N(c3ccccc3)c3ccc5c(c3)C(C)(C)CCC5(C)C)C3(c5ccccc5-4)C4CC5CC(C4)CC3C5)ccc21 ; CC1(C)CCC(C)(C)c2cc(-c3cc4c(cc3N(c3ccc(-c5ccccc5)cc3)c3ccc5c(c3)C(C)(C)CCC5(C)C)C3(c5ccccc5-4)C4CC5CC(C4)CC3C5)ccc21 ; CC1(C)CCC(C)(C)c2cc(-c3cc4c(cc3N(c3ccc5c(c3)C(C)(C)CCC5(C)C)c3ccc5c(c3)C(C)(C)c3ccccc3-5)C3(c5ccccc5-4)C4CC5CC(C4)CC3C5)ccc21]

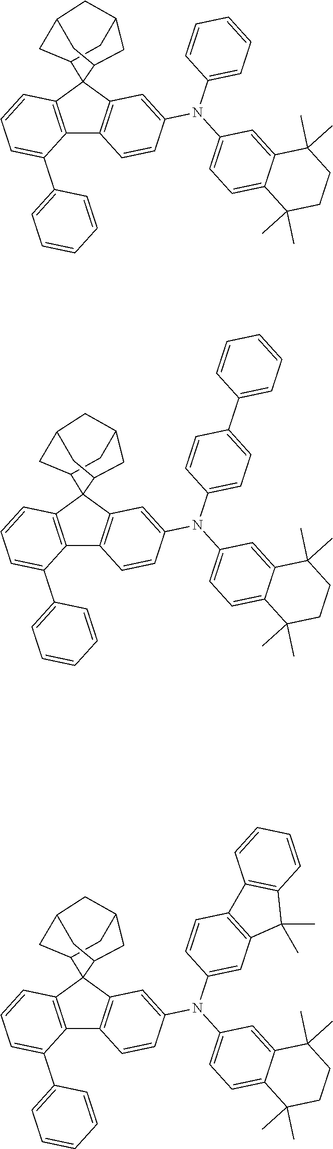 [SMILES: CC1(C)CCC(C)(C)c2cc(N(c3ccccc3)c3ccc4c(c3)C3(c5cccc(-c6ccccc6)c5-4)C4CC5CC(C4)CC3C5)ccc21 ; CC1(C)CCC(C)(C)c2cc(N(c3ccc(-c4ccccc4)cc3)c3ccc4c(c3)C3(c5cccc(-c6ccccc6)c5-4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5cccc(-c6ccccc6)c5-4)C4CC5CC(C4)CC3C5)ccc21]

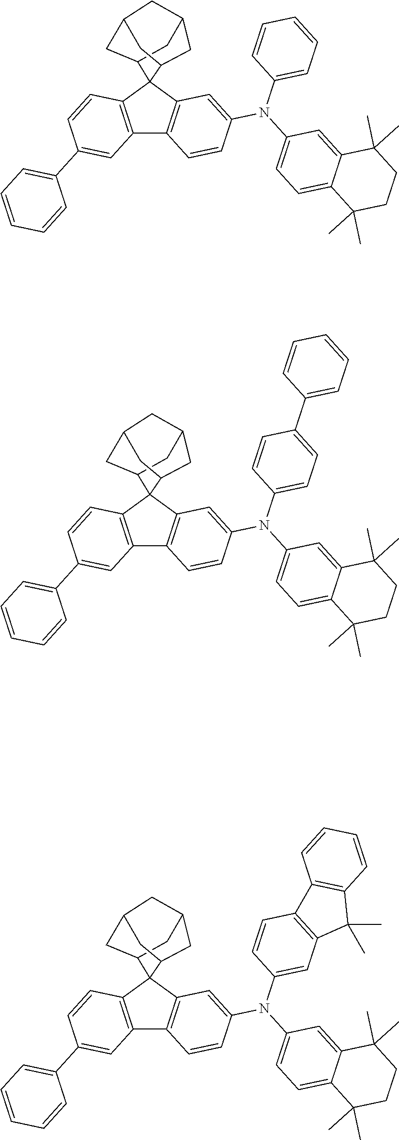 [SMILES: CC1(C)CCC(C)(C)c2cc(N(c3ccccc3)c3ccc4c(c3)C3(c5ccc(-c6ccccc6)cc5-4)C4CC5CC(C4)CC3C5)ccc21 ; CC1(C)CCC(C)(C)c2cc(N(c3ccc(-c4ccccc4)cc3)c3ccc4c(c3)C3(c5ccc(-c6ccccc6)cc5-4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5ccc(-c6ccccc6)cc5-4)C4CC5CC(C4)CC3C5)ccc21]

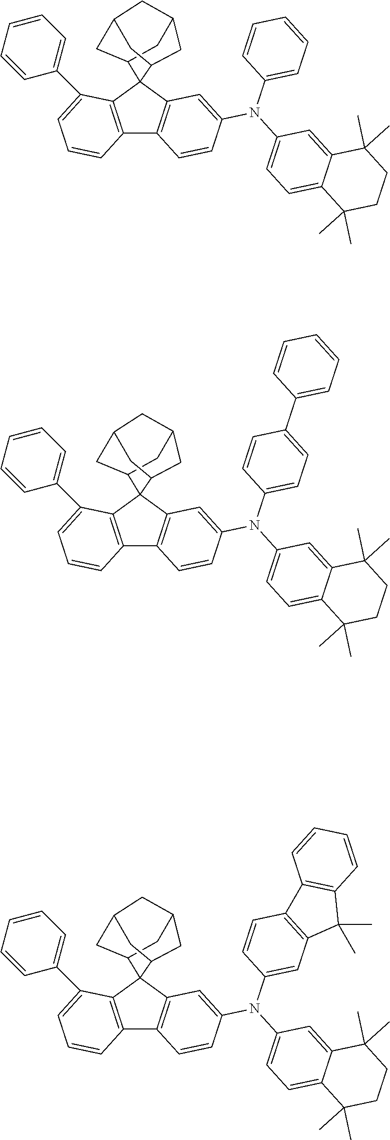 [SMILES: CC1(C)CCC(C)(C)c2cc(N(c3ccccc3)c3ccc4c(c3)C3(c5c(-c6ccccc6)cccc5-4)C4CC5CC(C4)CC3C5)ccc21 ; CC1(C)CCC(C)(C)c2cc(N(c3ccc(-c4ccccc4)cc3)c3ccc4c(c3)C3(c5c(-c6ccccc6)cccc5-4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5c(-c6ccccc6)cccc5-4)C4CC5CC(C4)CC3C5)ccc21]

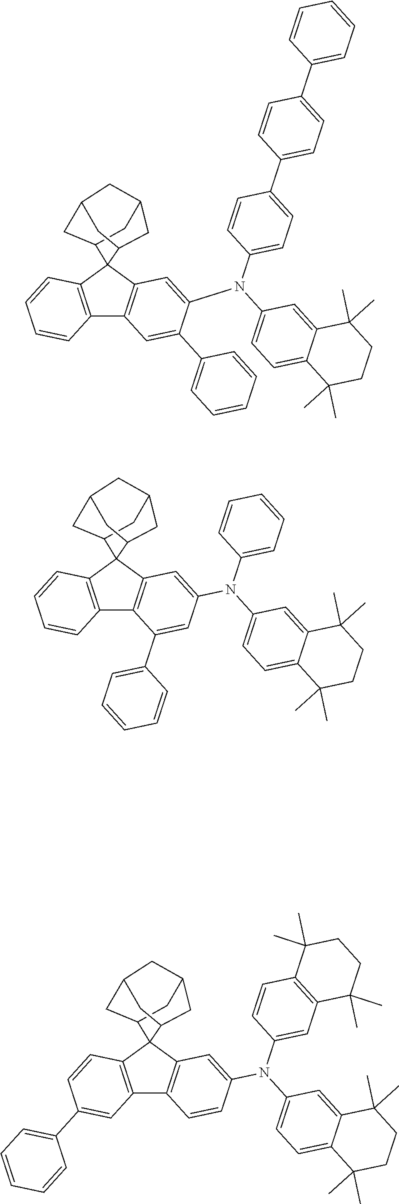 [SMILES: CC1(C)CCC(C)(C)c2cc(N(c3ccc(-c4ccc(-c5ccccc5)cc4)cc3)c3cc4c(cc3-c3ccccc3)-c3ccccc3C43C4CC5CC(C4)CC3C5)ccc21 ; CC1(C)CCC(C)(C)c2cc(N(c3ccccc3)c3cc(-c4ccccc4)c4c(c3)C3(c5ccccc5-4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5ccc(-c6ccccc6)cc5-4)C4CC5CC(C4)CC3C5)ccc21]

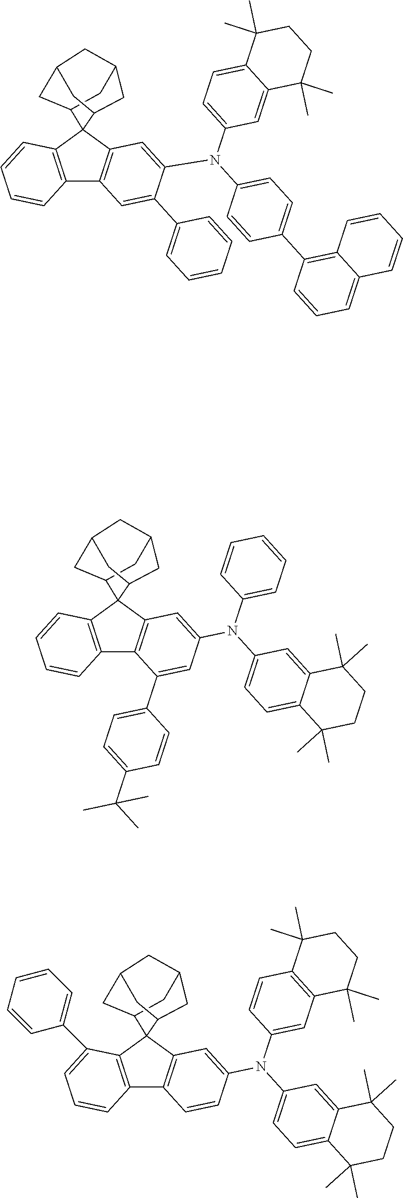 [SMILES: CC1(C)CCC(C)(C)c2cc(N(c3ccc(-c4cccc5ccccc45)cc3)c3cc4c(cc3-c3ccccc3)-c3ccccc3C43C4CC5CC(C4)CC3C5)ccc21 ; CC(C)(C)c1ccc(-c2cc(N(c3ccccc3)c3ccc4c(c3)C(C)(C)CCC4(C)C)cc3c2-c2ccccc2C32C3CC4CC(C3)CC2C4)cc1 ; CC1(C)CCC(C)(C)c2cc(N(c3ccc4c(c3)C(C)(C)CCC4(C)C)c3ccc4c(c3)C3(c5c(-c6ccccc6)cccc5-4)C4CC5CC(C4)CC3C5)ccc21]

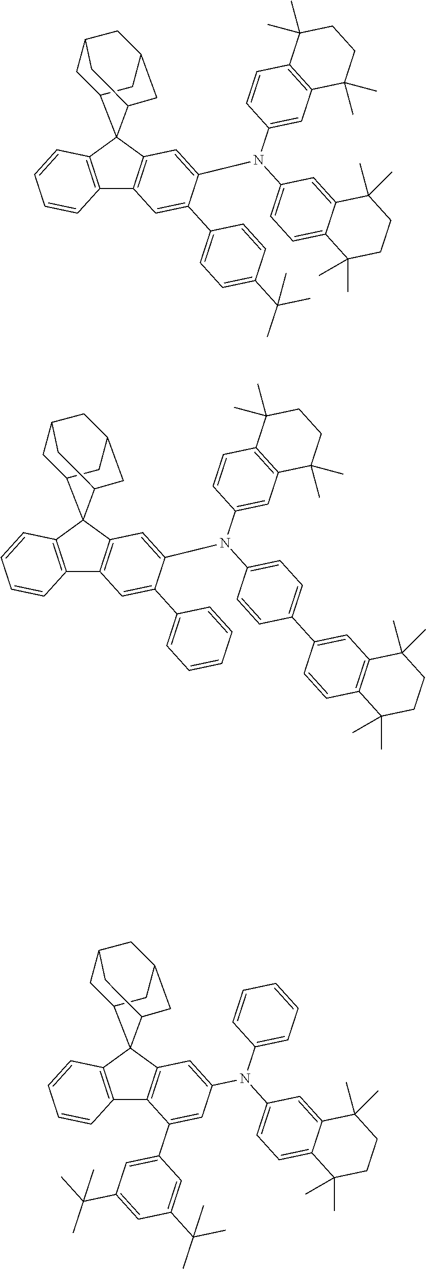 [SMILES: CC(C)(C)c1ccc(-c2cc3c(cc2N(c2ccc4c(c2)C(C)(C)CCC4(C)C)c2ccc4c(c2)C(C)(C)CCC4(C)C)C2(c4ccccc4-3)C3CC4CC(C3)CC2C4)cc1 ; CC1(C)CCC(C)(C)c2cc(-c3ccc(N(c4ccc5c(c4)C(C)(C)CCC5(C)C)c4cc5c(cc4-c4ccccc4)-c4ccccc4C54C5CC6CC(C5)CC4C6)cc3)ccc21 ; CC(C)(C)c1cc(-c2cc(N(c3ccccc3)c3ccc4c(c3)C(C)(C)CCC4(C)C)cc3c2-c2ccccc2C32C3CC4CC(C3)CC2C4)cc(C(C)(C)C)c1]

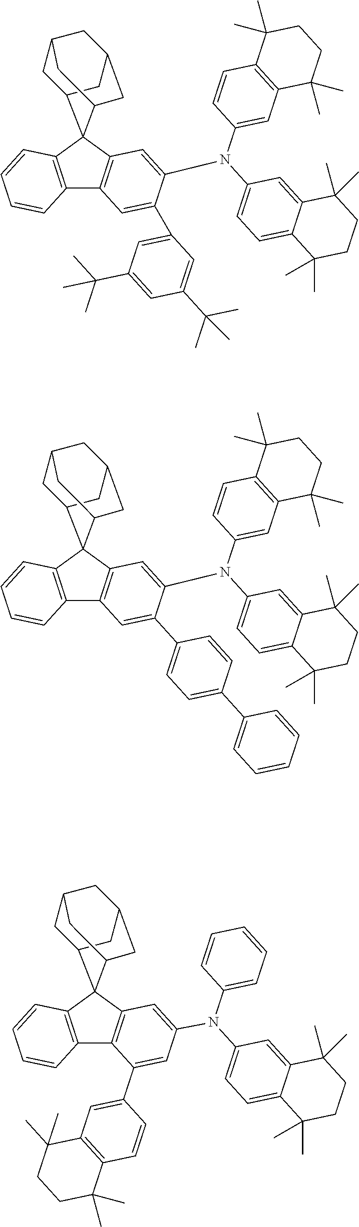 [SMILES: CC(C)(C)c1cc(-c2cc3c(cc2N(c2ccc4c(c2)C(C)(C)CCC4(C)C)c2ccc4c(c2)C(C)(C)CCC4(C)C)C2(c4ccccc4-3)C3CC4CC(C3)CC2C4)cc(C(C)(C)C)c1 ; CC1(C)CCC(C)(C)c2cc(N(c3ccc4c(c3)C(C)(C)CCC4(C)C)c3cc4c(cc3-c3ccc(-c5ccccc5)cc3)-c3ccccc3C43C4CC5CC(C4)CC3C5)ccc21 ; CC1(C)CCC(C)(C)c2cc(-c3cc(N(c4ccccc4)c4ccc5c(c4)C(C)(C)CCC5(C)C)cc4c3-c3ccccc3C43C4CC5CC(C4)CC3C5)ccc21]

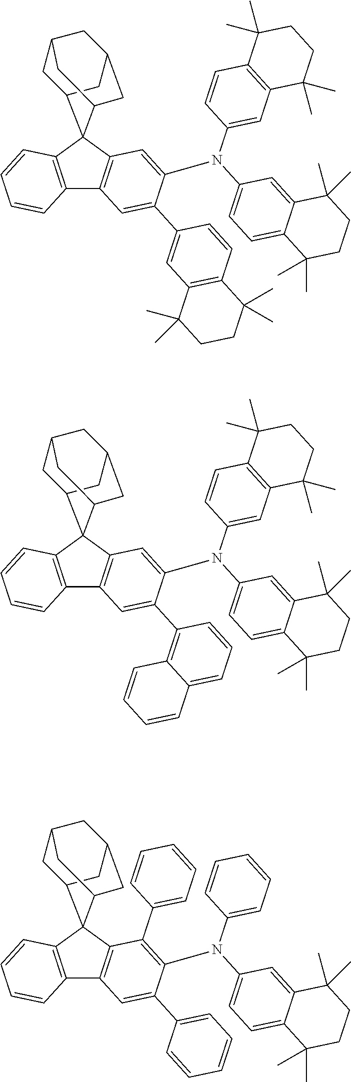 [SMILES: CC1(C)CCC(C)(C)c2cc(-c3cc4c(cc3N(c3ccc5c(c3)C(C)(C)CCC5(C)C)c3ccc5c(c3)C(C)(C)CCC5(C)C)C3(c5ccccc5-4)C4CC5CC(C4)CC3C5)ccc21 ; CC1(C)CCC(C)(C)c2cc(N(c3ccc4c(c3)C(C)(C)CCC4(C)C)c3cc4c(cc3-c3cccc5ccccc35)-c3ccccc3C43C4CC5CC(C4)CC3C5)ccc21 ; CC1(C)CCC(C)(C)c2cc(N(c3ccccc3)c3c(-c4ccccc4)cc4c(c3-c3ccccc3)C3(c5ccccc5-4)C4CC5CC(C4)CC3C5)ccc21]

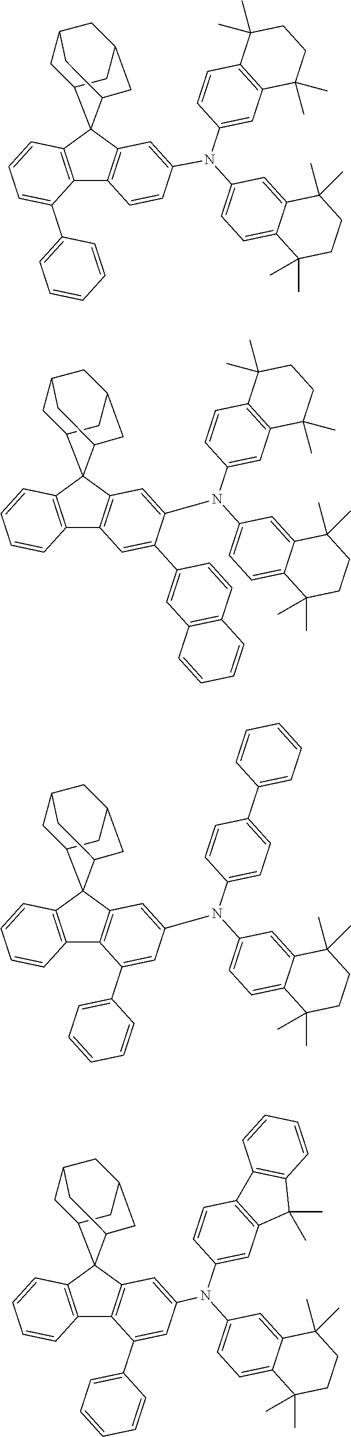 [SMILES: CC1(C)CCC(C)(C)c2cc(N(c3ccc4c(c3)C(C)(C)CCC4(C)C)c3ccc4c(c3)C3(c5cccc(-c6ccccc6)c5-4)C4CC5CC(C4)CC3C5)ccc21 ; CC1(C)CCC(C)(C)c2cc(N(c3ccc4c(c3)C(C)(C)CCC4(C)C)c3cc4c(cc3-c3ccc5ccccc5c3)-c3ccccc3C43C4CC5CC(C4)CC3C5)ccc21 ; CC1(C)CCC(C)(C)c2cc(N(c3ccc(-c4ccccc4)cc3)c3cc(-c4ccccc4)c4c(c3)C3(c5ccccc5-4)C4CC5CC(C4)CC3C5)ccc21 ; CC1(C)CCC(C)(C)c2cc(N(c3ccc4c(c3)C(C)(C)c3ccccc3-4)c3cc(-c4ccccc4)c4c(c3)C3(c5ccccc5-4)C4CC5CC(C4)CC3C5)ccc21]

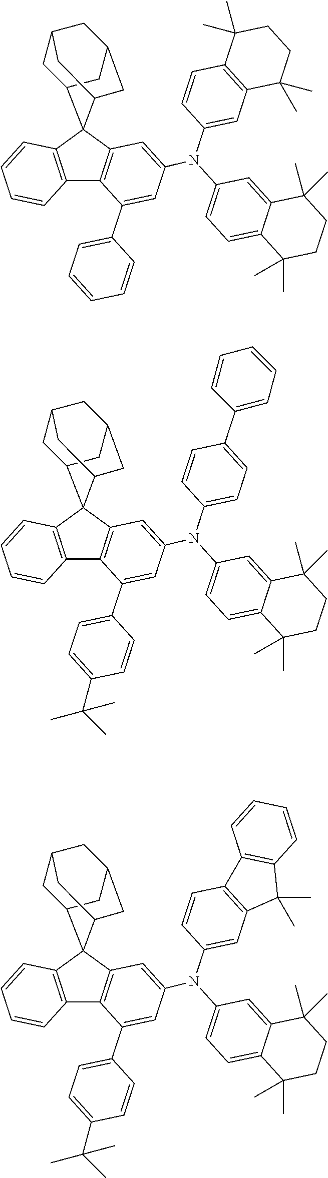 [SMILES: CC1(C)CCC(C)(C)c2cc(N(c3ccc4c(c3)C(C)(C)CCC4(C)C)c3cc(-c4ccccc4)c4c(c3)C3(c5ccccc5-4)C4CC5CC(C4)CC3C5)ccc21 ; CC(C)(C)c1ccc(-c2cc(N(c3ccc(-c4ccccc4)cc3)c3ccc4c(c3)C(C)(C)CCC4(C)C)cc3c2-c2ccccc2C32C3CC4CC(C3)CC2C4)cc1 ; CC(C)(C)c1ccc(-c2cc(N(c3ccc4c(c3)C(C)(C)CCC4(C)C)c3ccc4c(c3)C(C)(C)c3ccccc3-4)cc3c2-c2ccccc2C32C3CC4CC(C3)CC2C4)cc1]

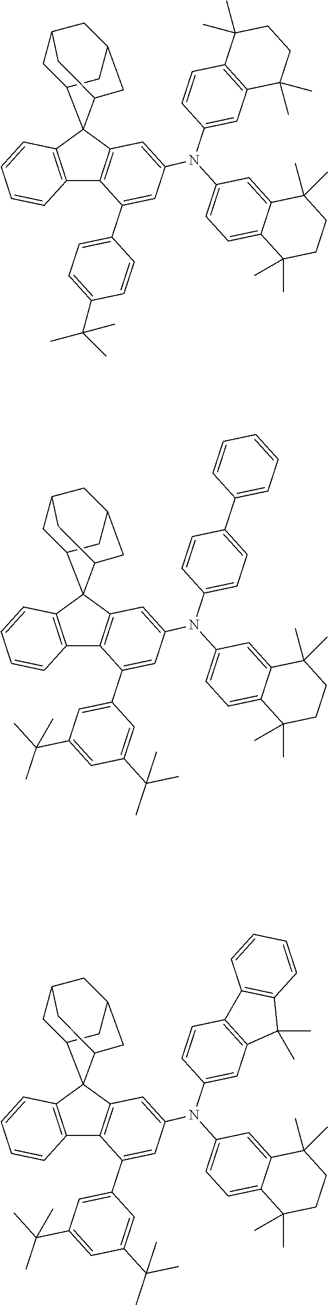 [SMILES: CC(C)(C)c1ccc(-c2cc(N(c3ccc4c(c3)C(C)(C)CCC4(C)C)c3ccc4c(c3)C(C)(C)CCC4(C)C)cc3c2-c2ccccc2C32C3CC4CC(C3)CC2C4)cc1 ; CC(C)(C)c1cc(-c2cc(N(c3ccc(-c4ccccc4)cc3)c3ccc4c(c3)C(C)(C)CCC4(C)C)cc3c2-c2ccccc2C32C3CC4CC(C3)CC2C4)cc(C(C)(C)C)c1 ; CC(C)(C)c1cc(-c2cc(N(c3ccc4c(c3)C(C)(C)CCC4(C)C)c3ccc4c(c3)C(C)(C)c3ccccc3-4)cc3c2-c2ccccc2C32C3CC4CC(C3)CC2C4)cc(C(C)(C)C)c1]

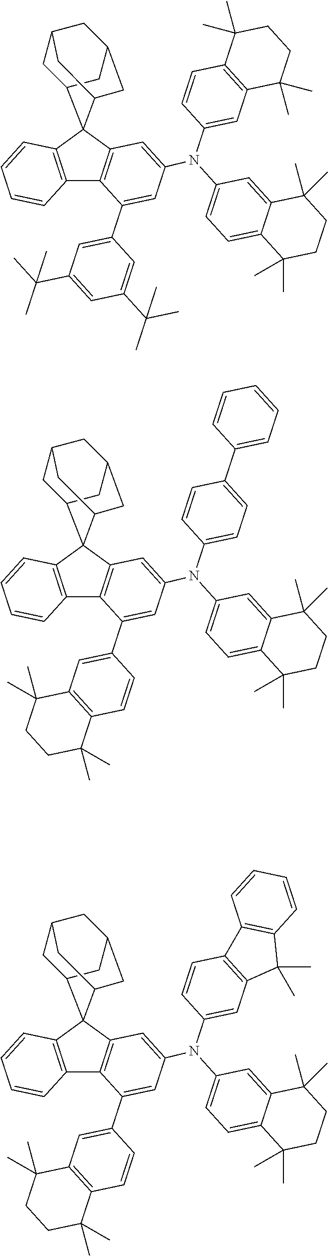 [SMILES: CC(C)(C)c1cc(-c2cc(N(c3ccc4c(c3)C(C)(C)CCC4(C)C)c3ccc4c(c3)C(C)(C)CCC4(C)C)cc3c2-c2ccccc2C32C3CC4CC(C3)CC2C4)cc(C(C)(C)C)c1 ; CC1(C)CCC(C)(C)c2cc(-c3cc(N(c4ccc(-c5ccccc5)cc4)c4ccc5c(c4)C(C)(C)CCC5(C)C)cc4c3-c3ccccc3C43C4CC5CC(C4)CC3C5)ccc21 ; CC1(C)CCC(C)(C)c2cc(-c3cc(N(c4ccc5c(c4)C(C)(C)CCC5(C)C)c4ccc5c(c4)C(C)(C)c4ccccc4-5)cc4c3-c3ccccc3C43C4CC5CC(C4)CC3C5)ccc21]

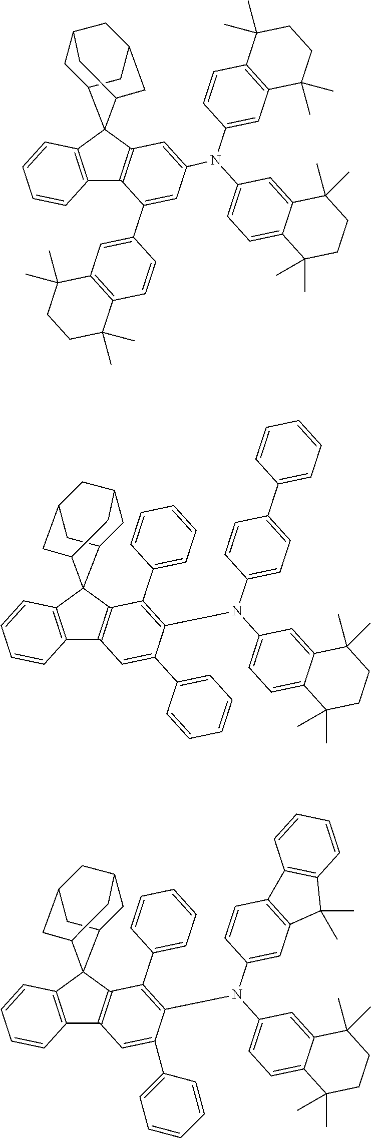 [SMILES: CC1(C)CCC(C)(C)c2cc(-c3cc(N(c4ccc5c(c4)C(C)(C)CCC5(C)C)c4ccc5c(c4)C(C)(C)CCC5(C)C)cc4c3-c3ccccc3C43C4CC5CC(C4)CC3C5)ccc21 ; CC1(C)CCC(C)(C)c2cc(N(c3ccc(-c4ccccc4)cc3)c3c(-c4ccccc4)cc4c(c3-c3ccccc3)C3(c5ccccc5-4)C4CC5CC(C4)CC3C5)ccc21 ; CC1(C)CCC(C)(C)c2cc(N(c3ccc4c(c3)C(C)(C)c3ccccc3-4)c3c(-c4ccccc4)cc4c(c3-c3ccccc3)C3(c5ccccc5-4)C4CC5CC(C4)CC3C5)ccc21]

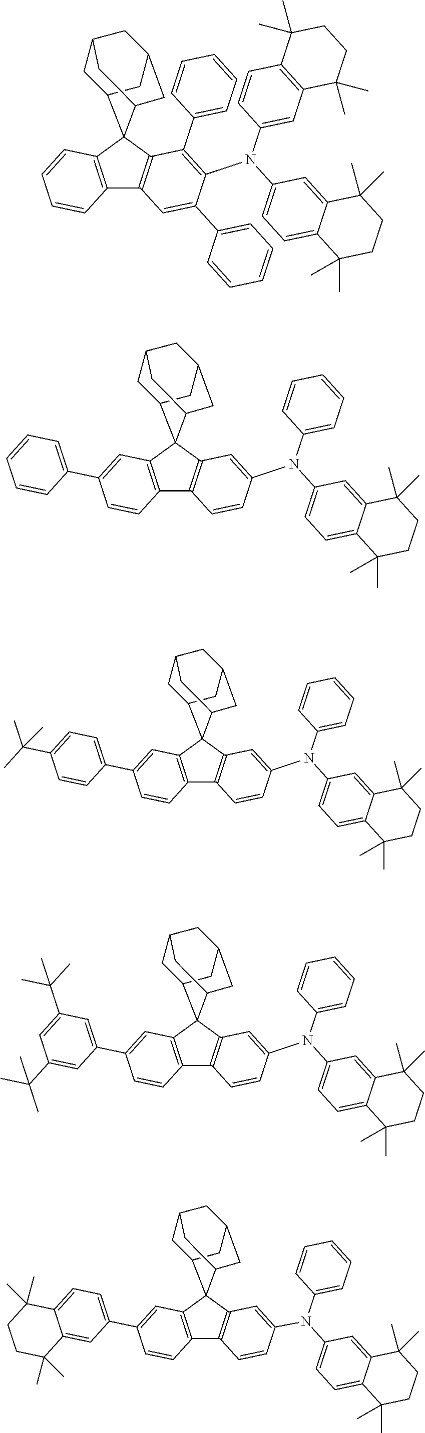

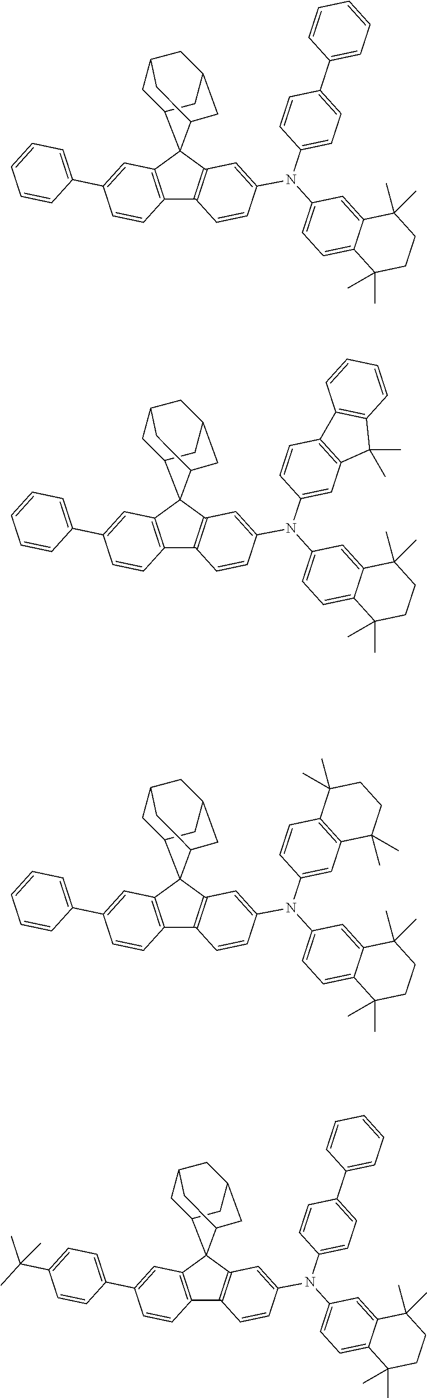

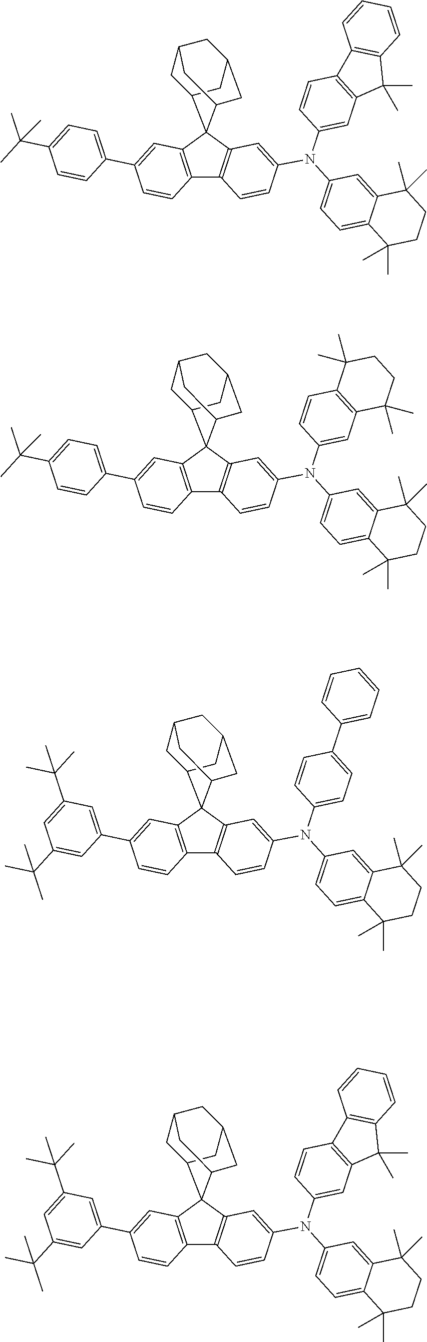

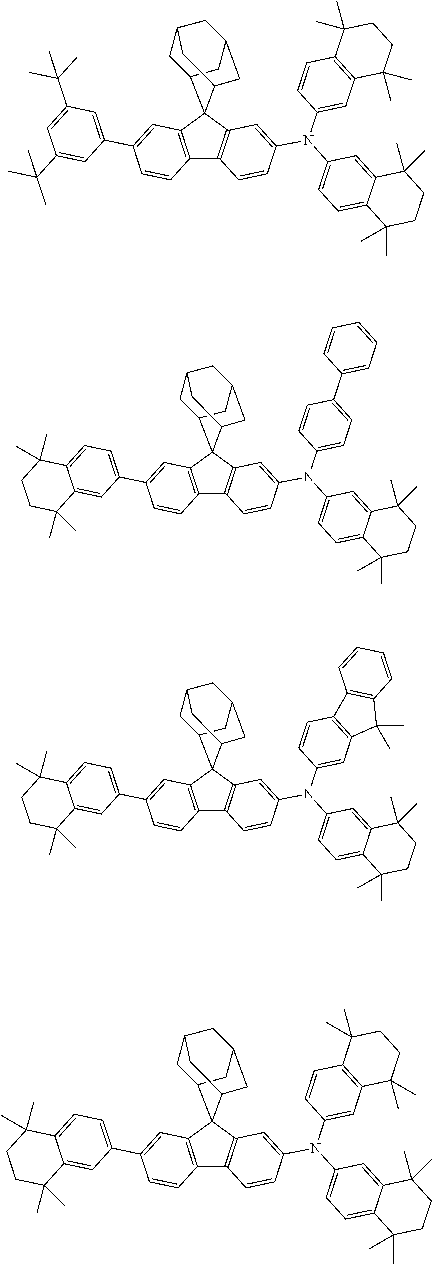

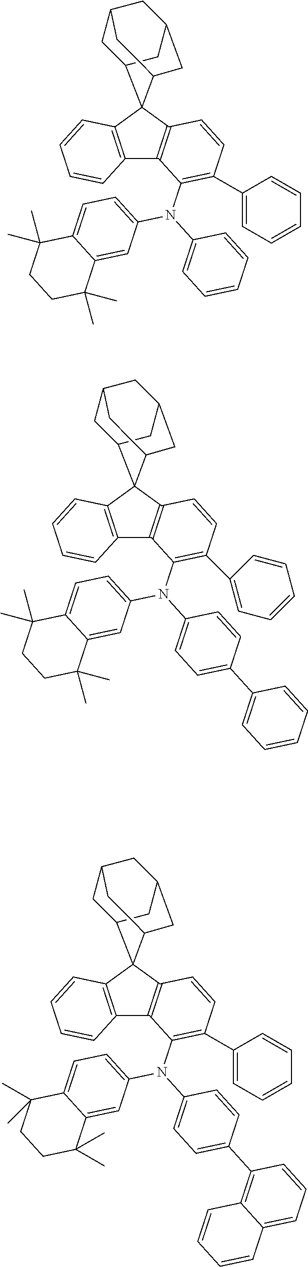 [SMILES: CC1(C)CCC(C)(C)c2cc(N(c3ccccc3)c3c(-c4ccccc4)ccc4c3-c3ccccc3C43C4CC5CC(C4)CC3C5)ccc21 ; CC1(C)CCC(C)(C)c2cc(N(c3ccc(-c4ccccc4)cc3)c3c(-c4ccccc4)ccc4c3-c3ccccc3C43C4CC5CC(C4)CC3C5)ccc21 ; CC1(C)CCC(C)(C)c2cc(N(c3ccc(-c4cccc5ccccc45)cc3)c3c(-c4ccccc4)ccc4c3-c3ccccc3C43C4CC5CC(C4)CC3C5)ccc21]

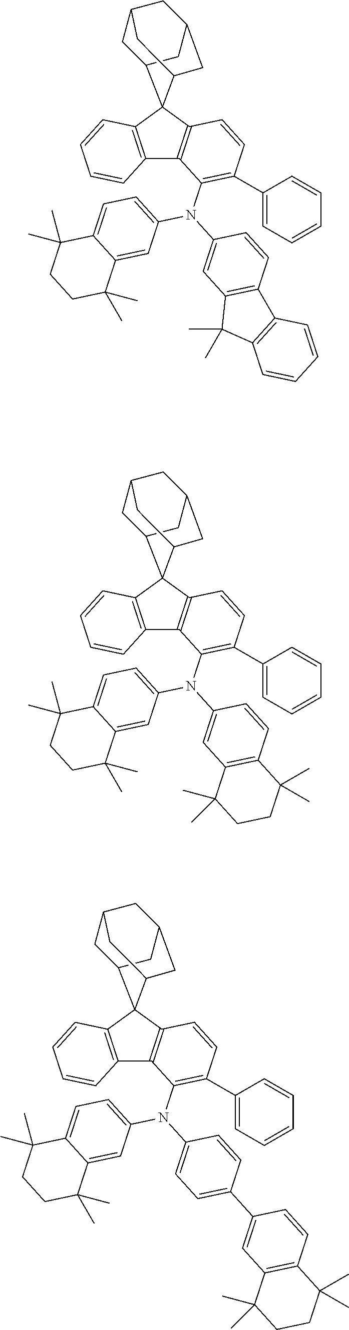

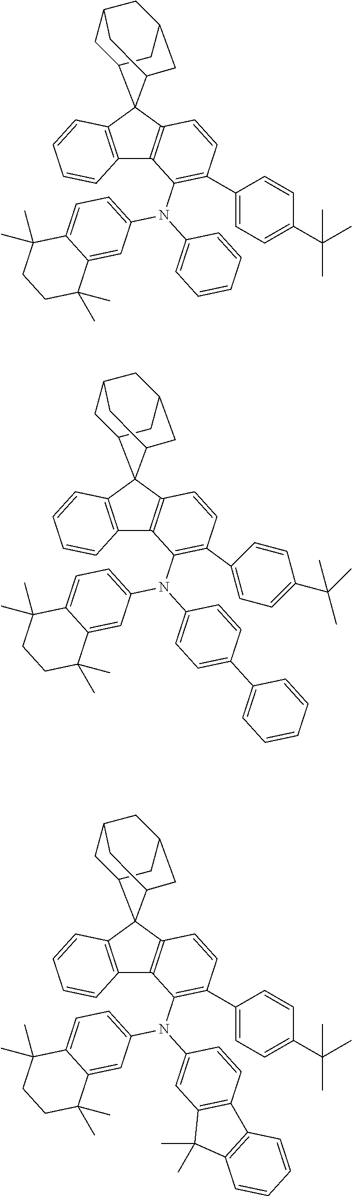

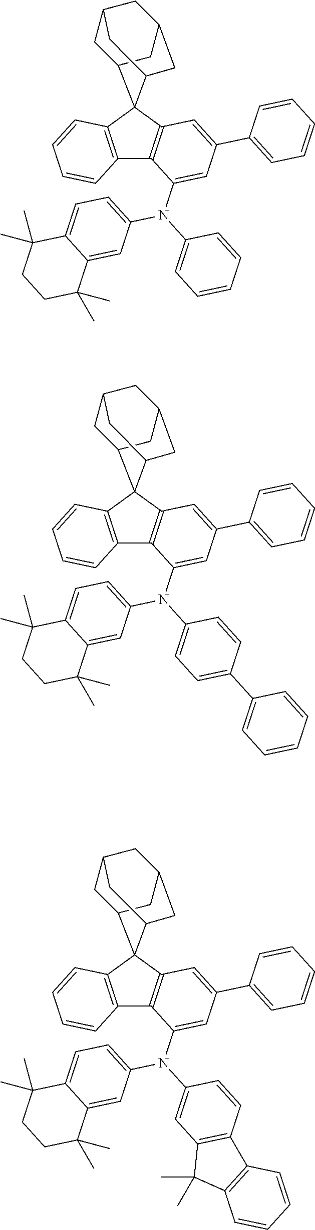 [SMILES: CC1(C)CCC(C)(C)c2cc(N(c3ccccc3)c3cc(-c4ccccc4)cc4c3-c3ccccc3C43C4CC5CC(C4)CC3C5)ccc21 ; CC1(C)CCC(C)(C)c2cc(N(c3ccc(-c4ccccc4)cc3)c3cc(-c4ccccc4)cc4c3-c3ccccc3C43C4CC5CC(C4)CC3C5)ccc21 ; CC1(C)CCC(C)(C)c2cc(N(c3ccc4c(c3)C(C)(C)c3ccccc3-4)c3cc(-c4ccccc4)cc4c3-c3ccccc3C43C4CC5CC(C4)CC3C5)ccc21]

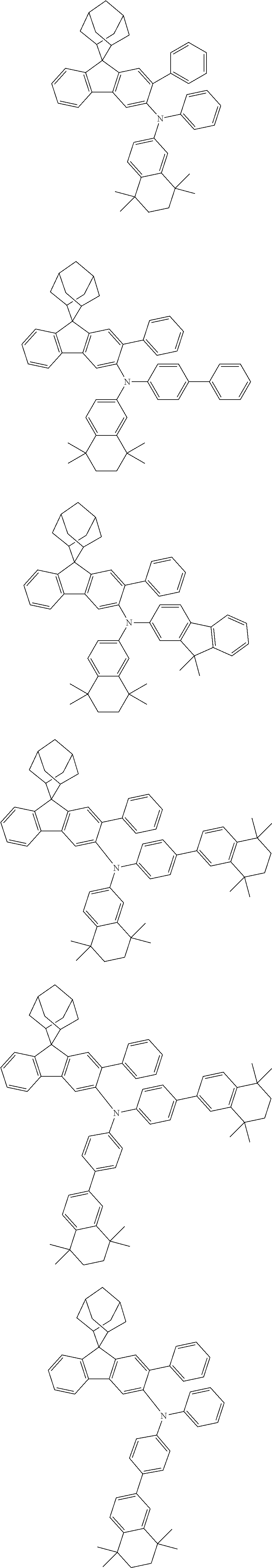

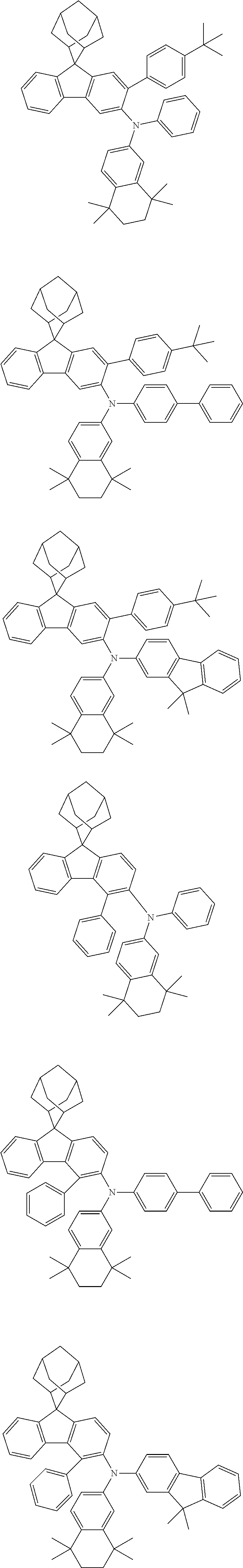

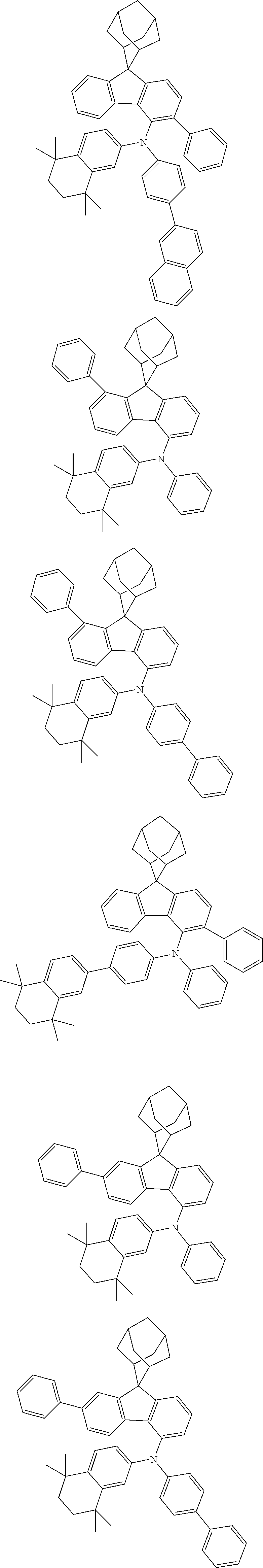

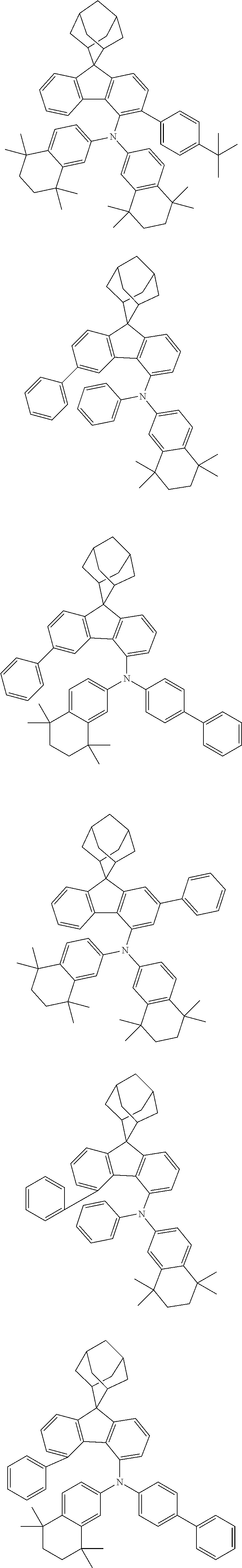

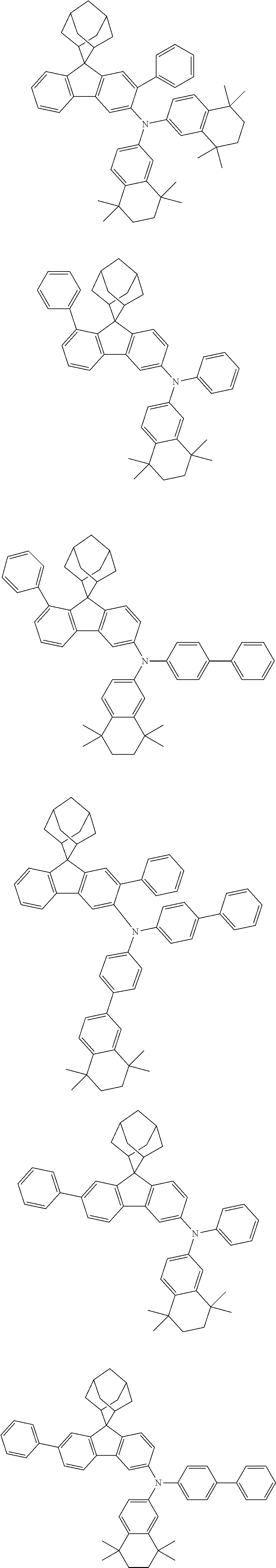

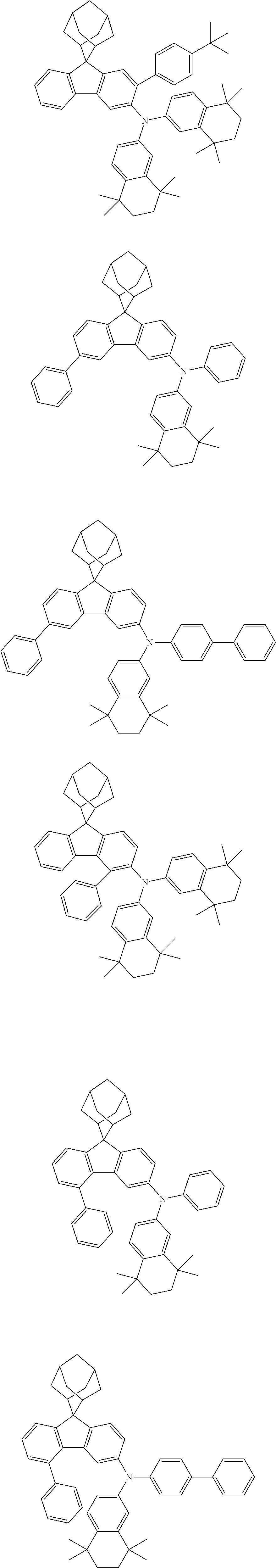

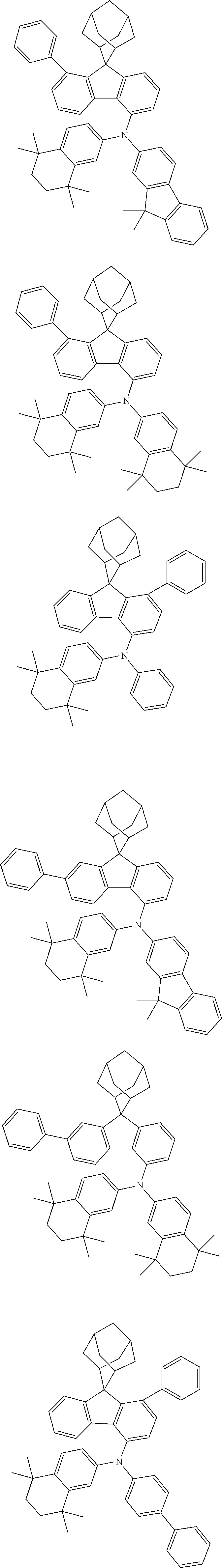

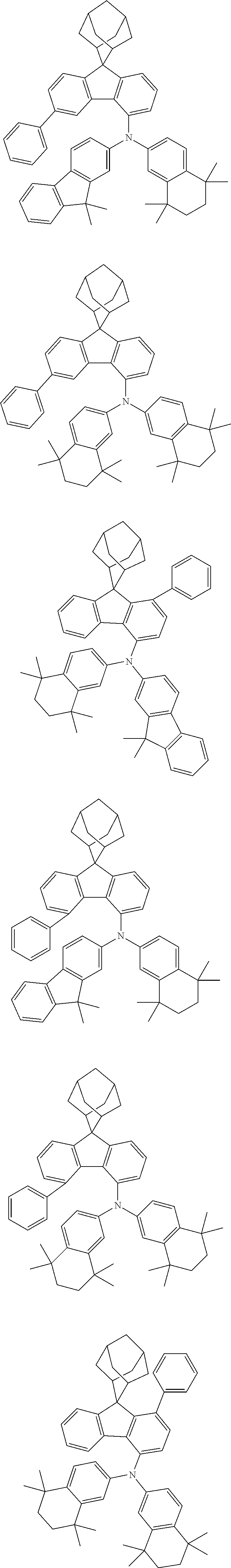

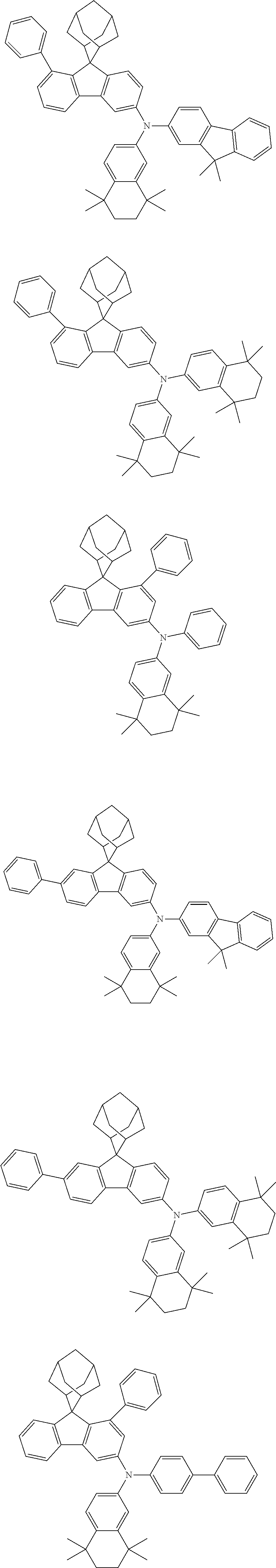

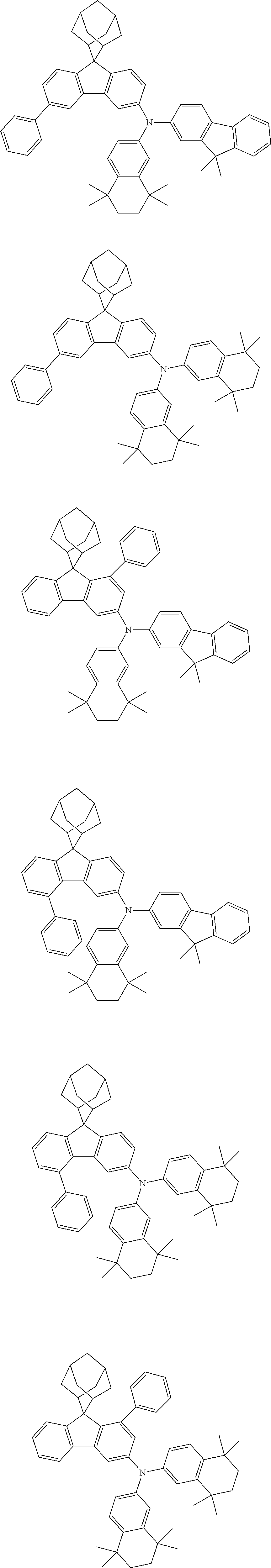

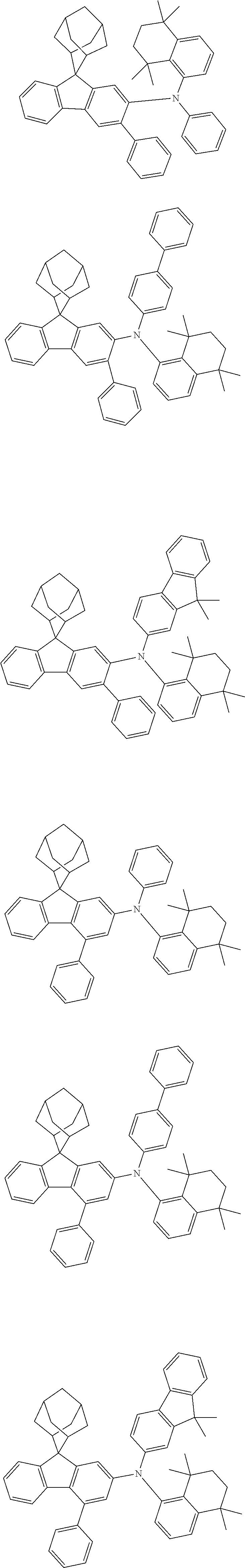

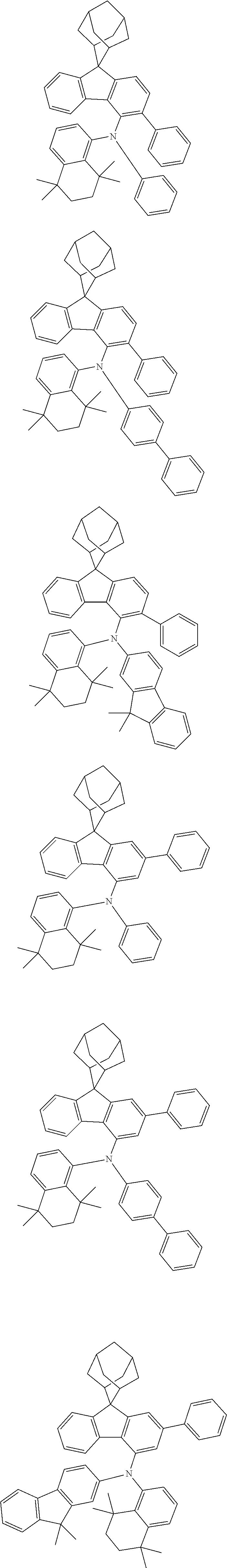

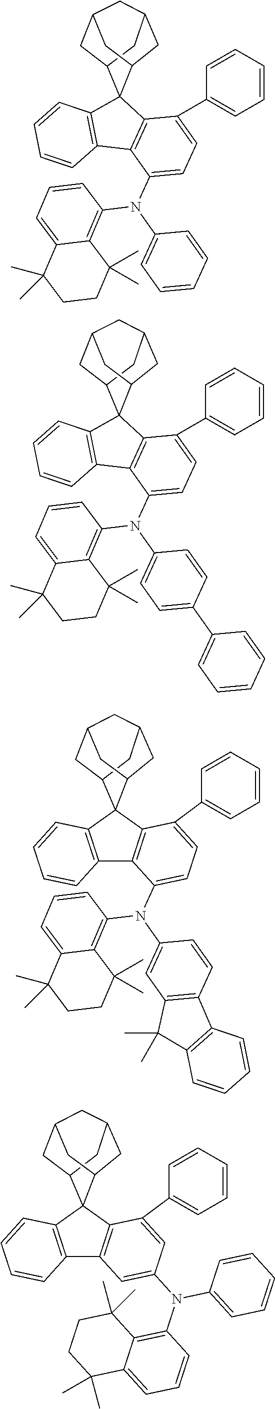

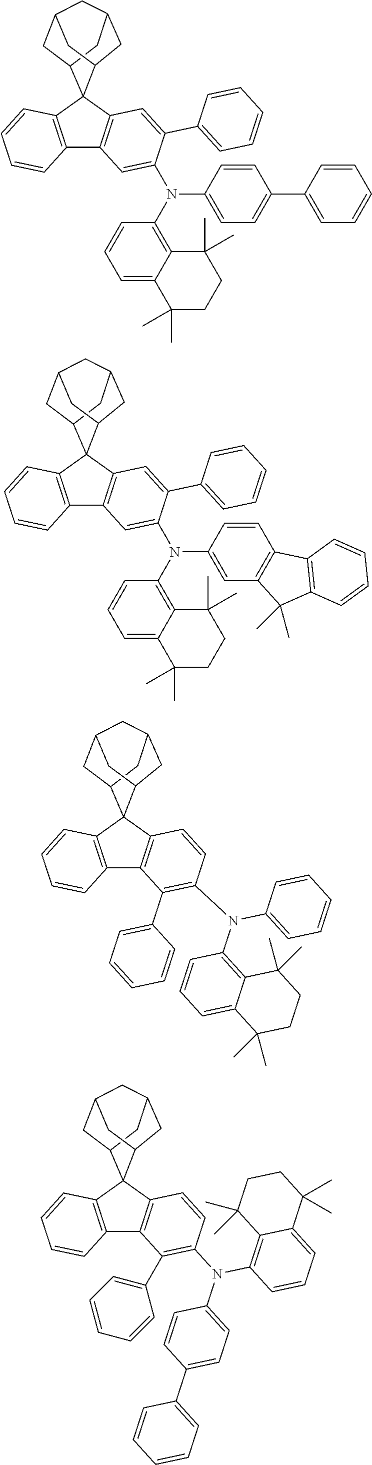

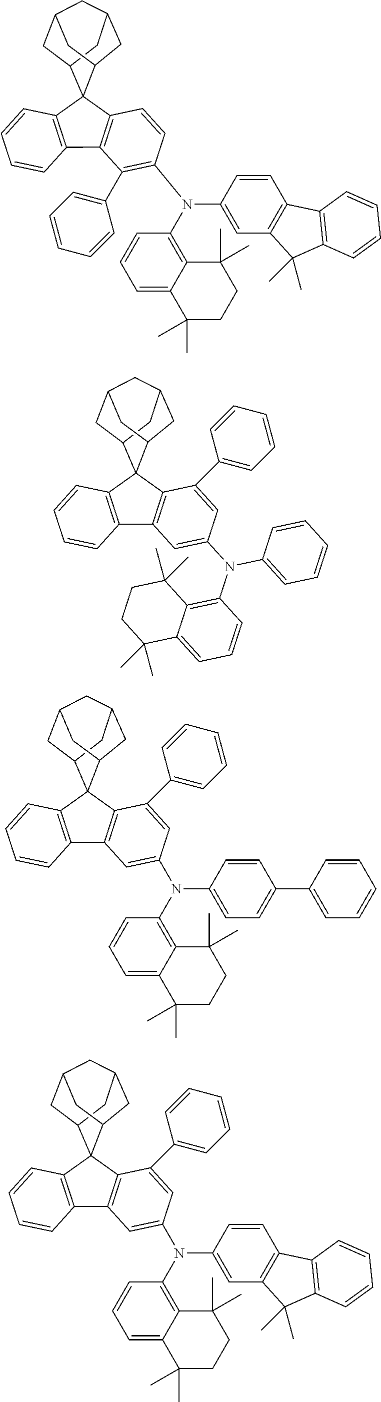

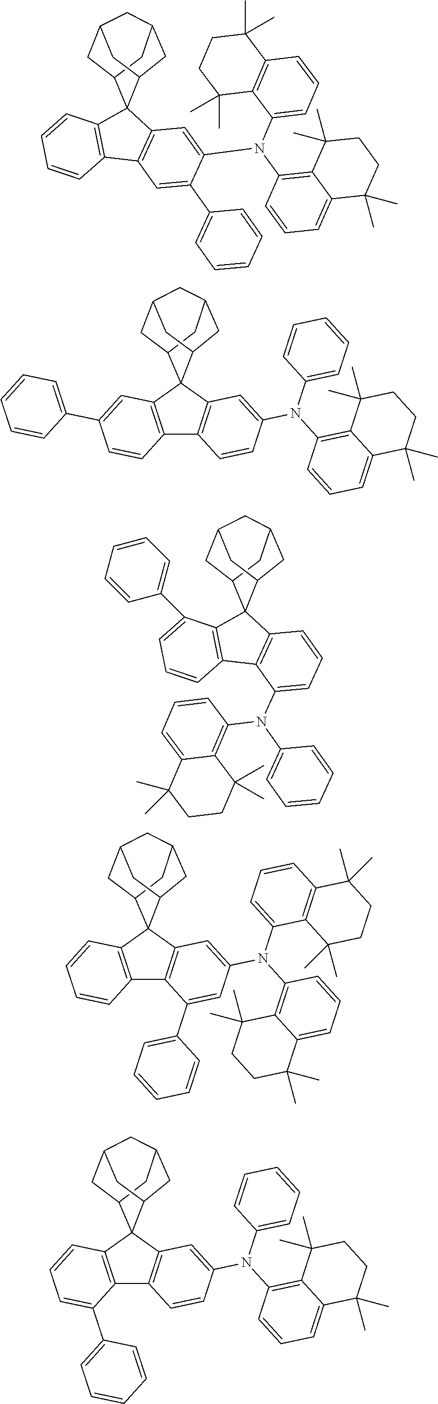

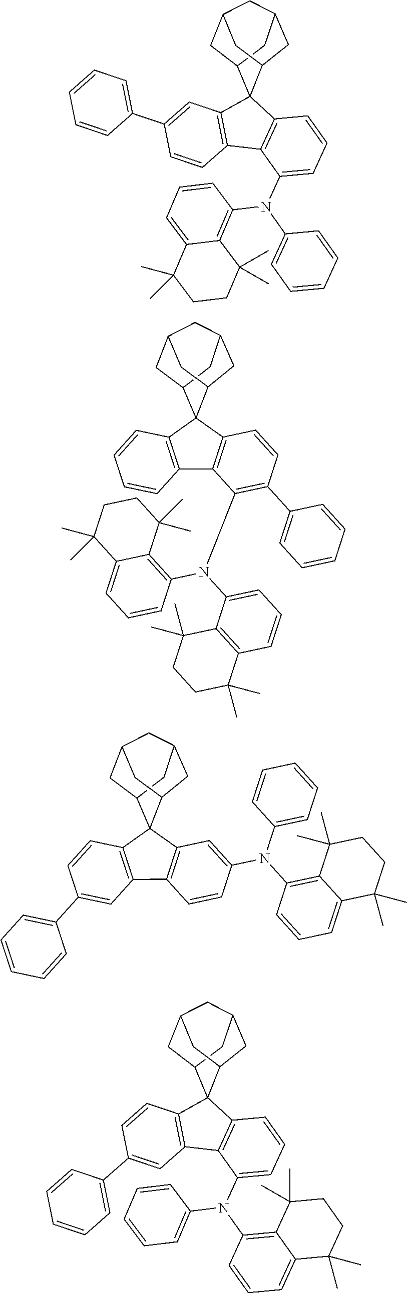

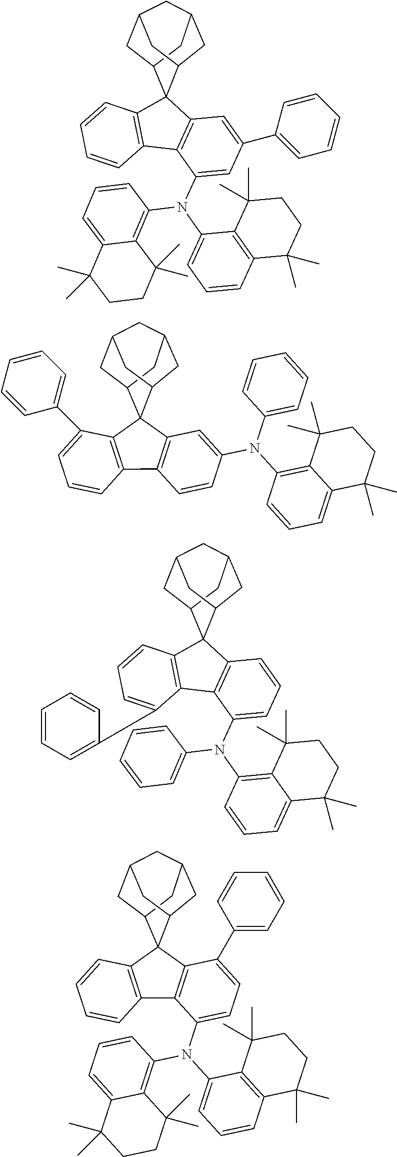

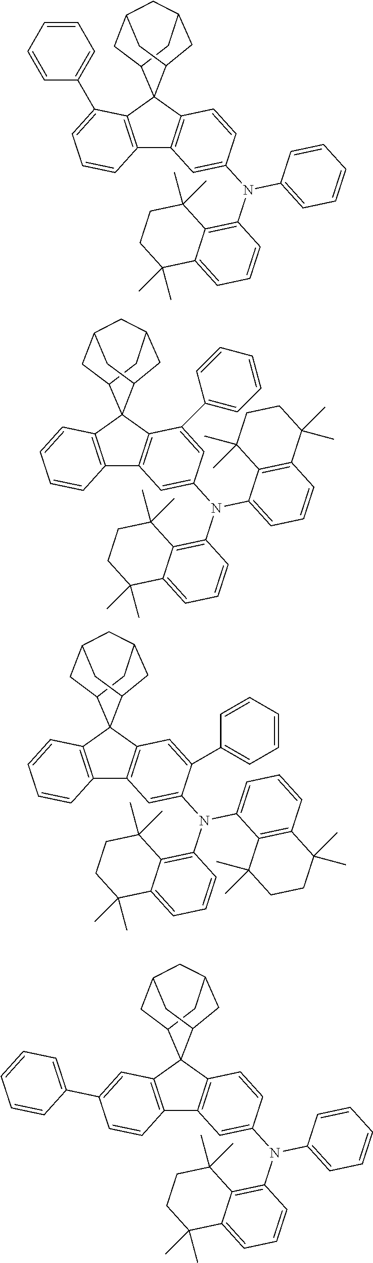

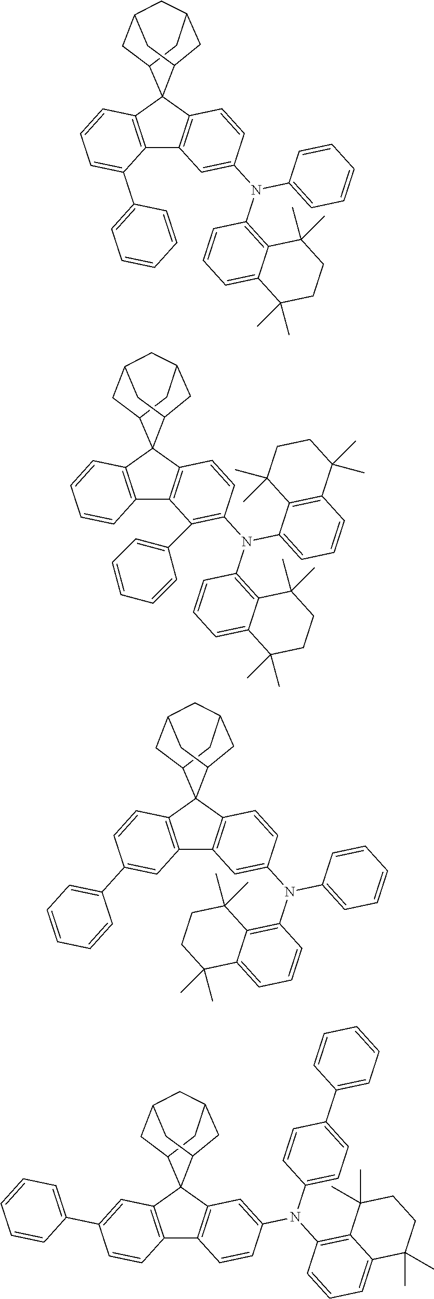

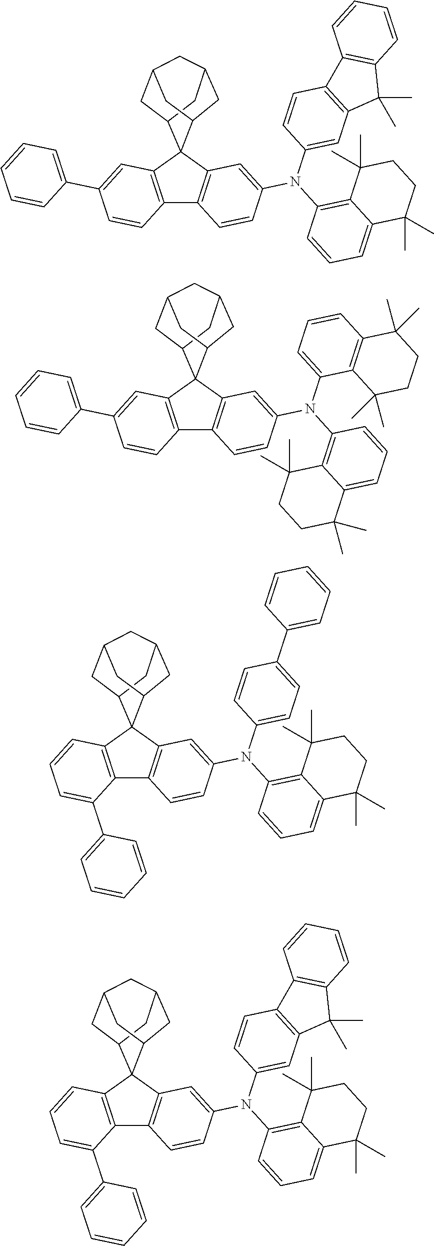

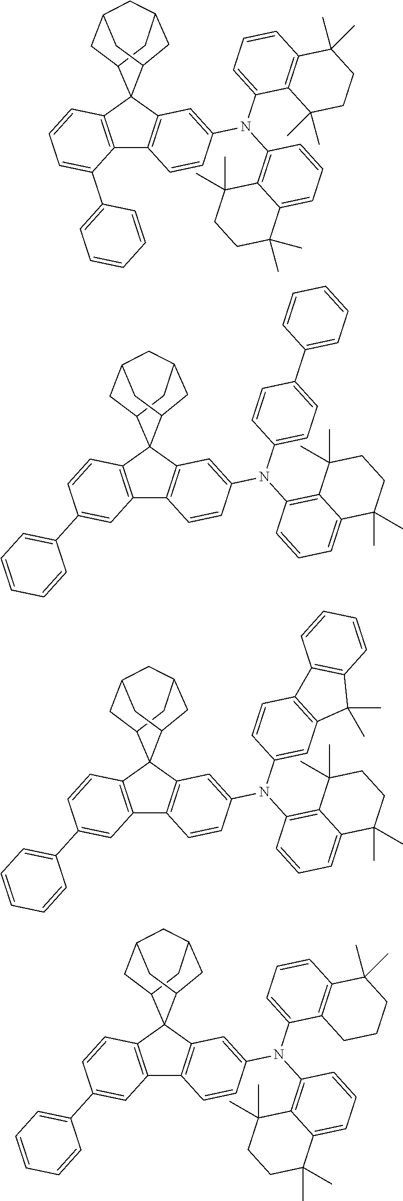

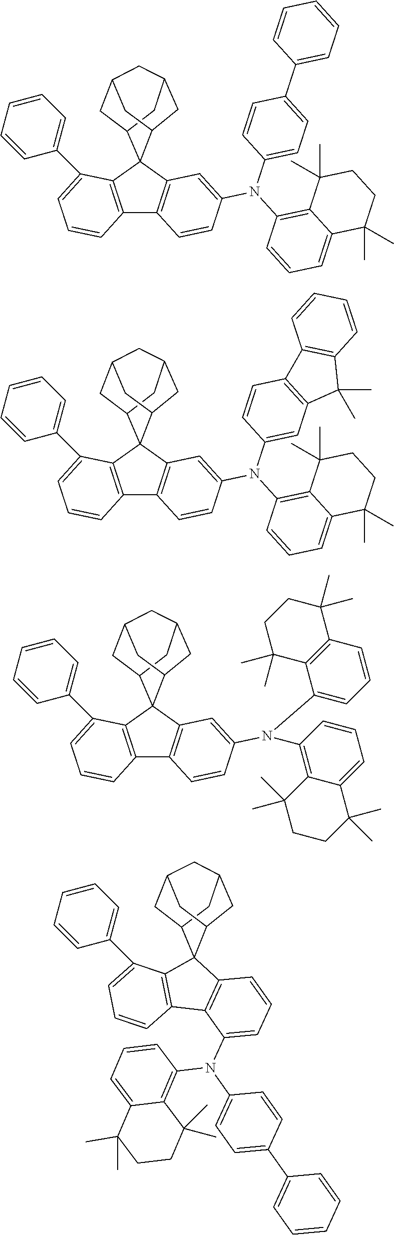

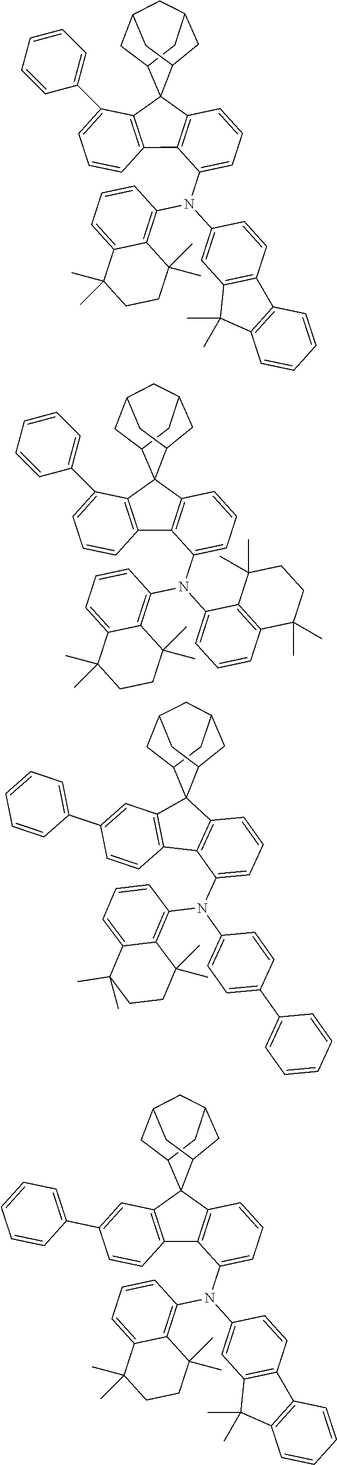

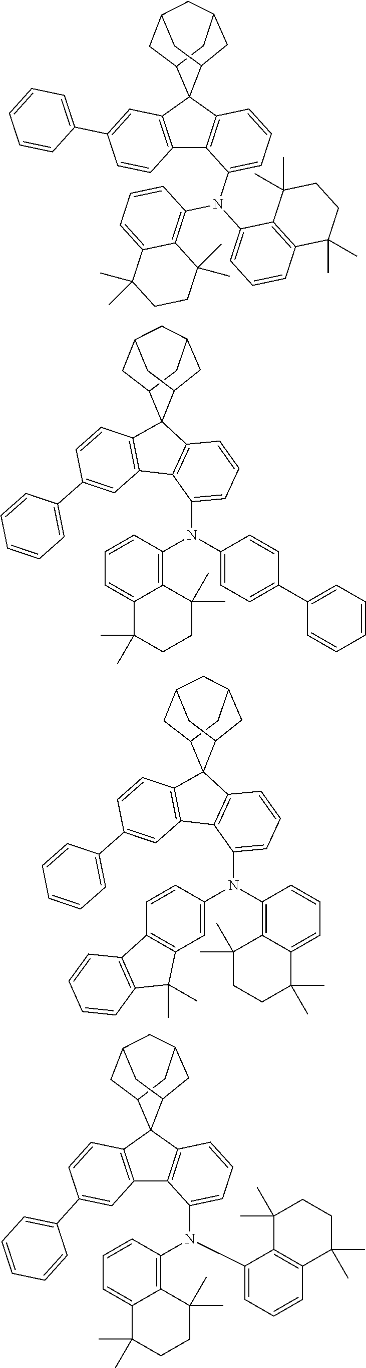

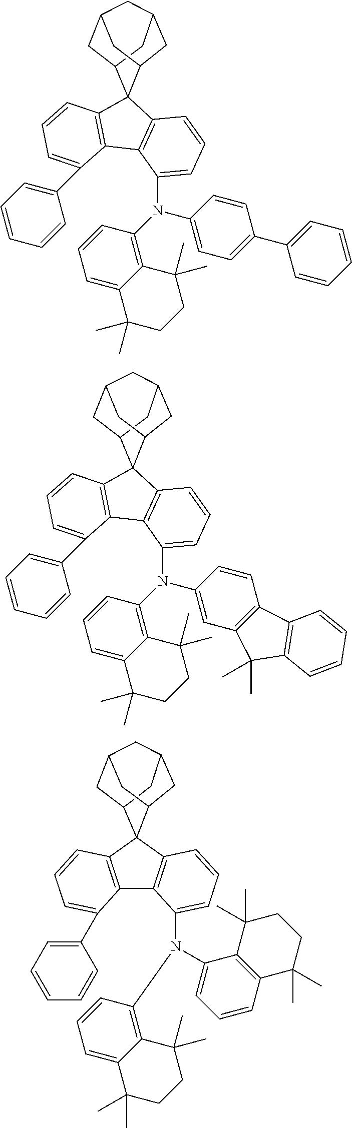

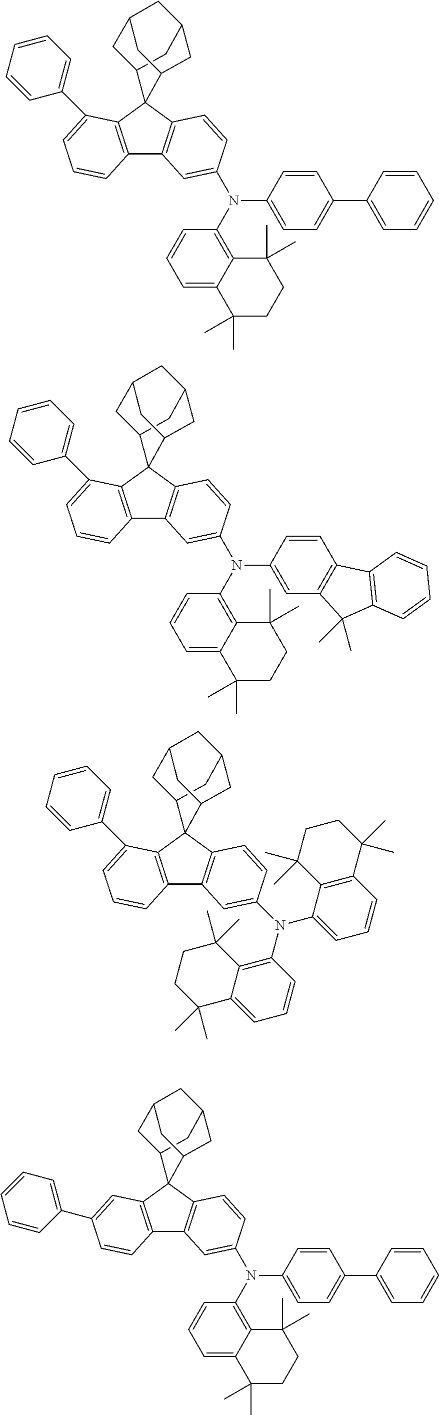

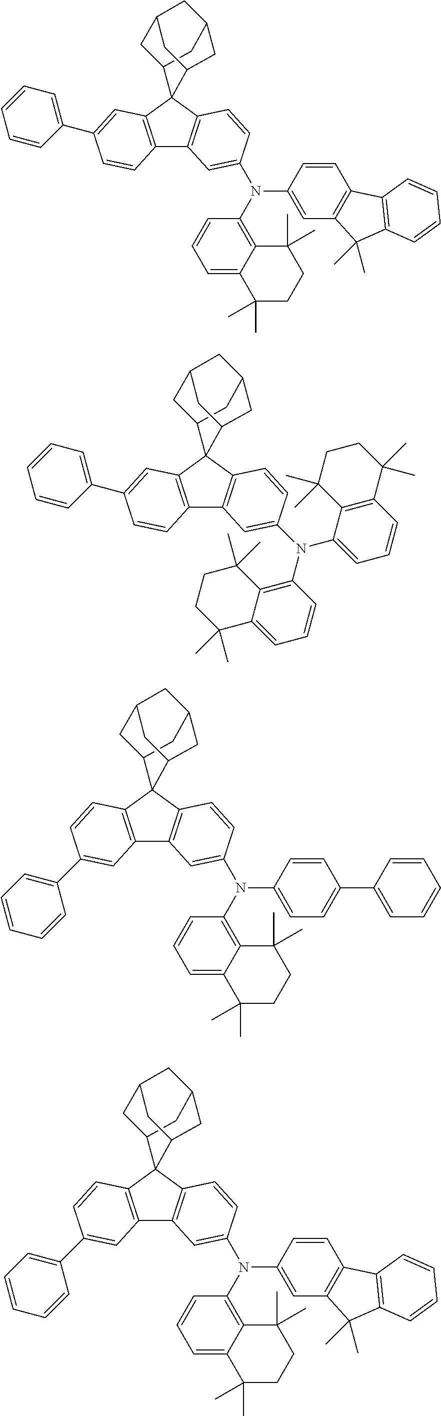

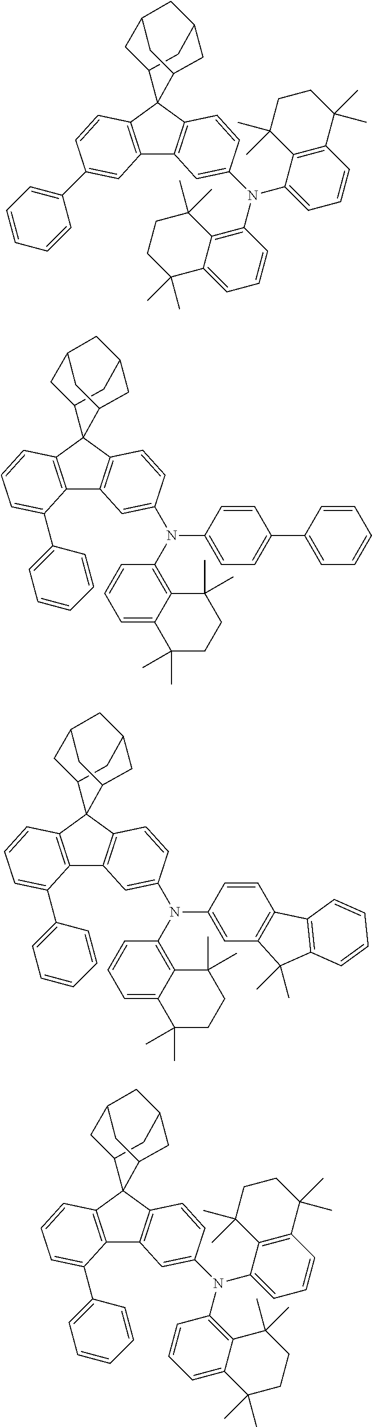

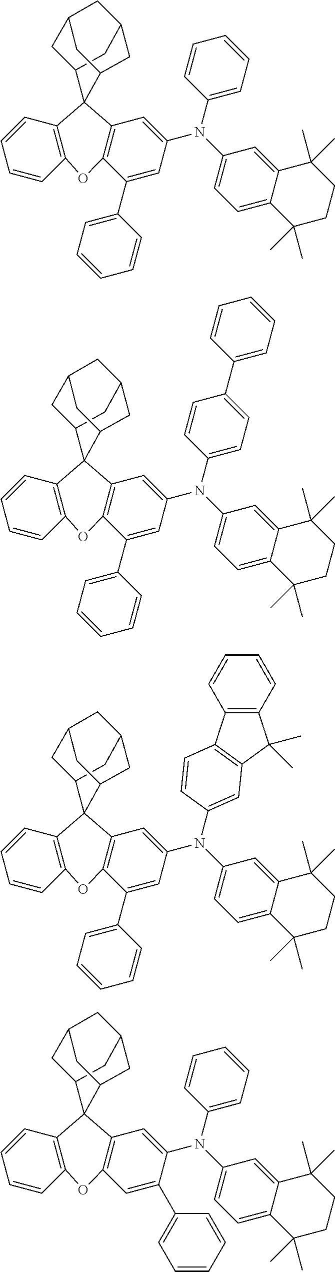

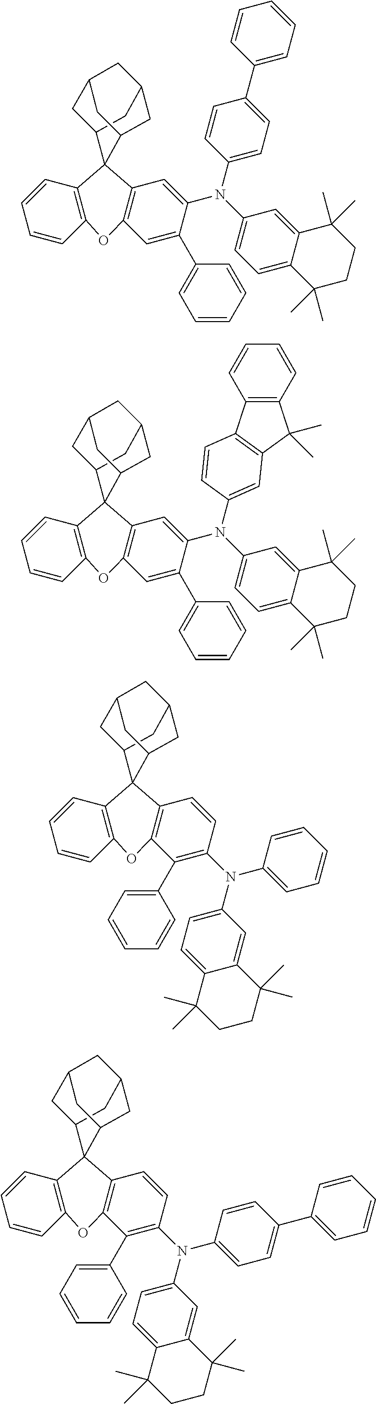

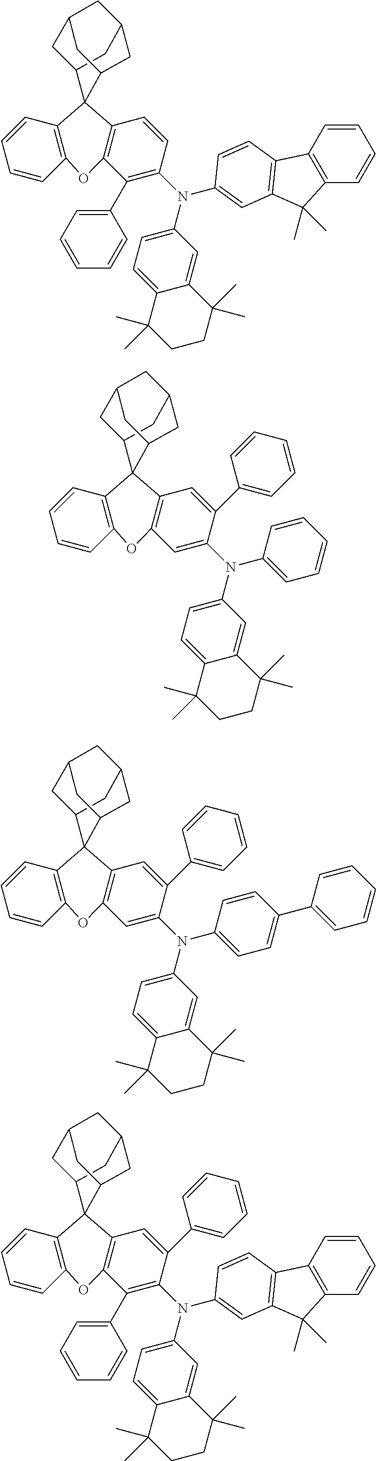

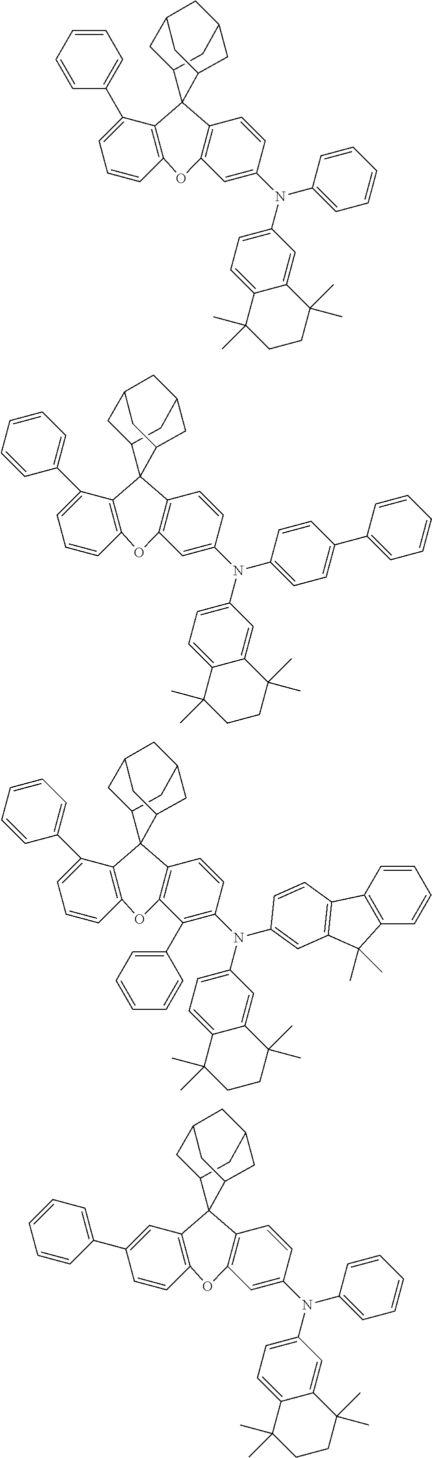

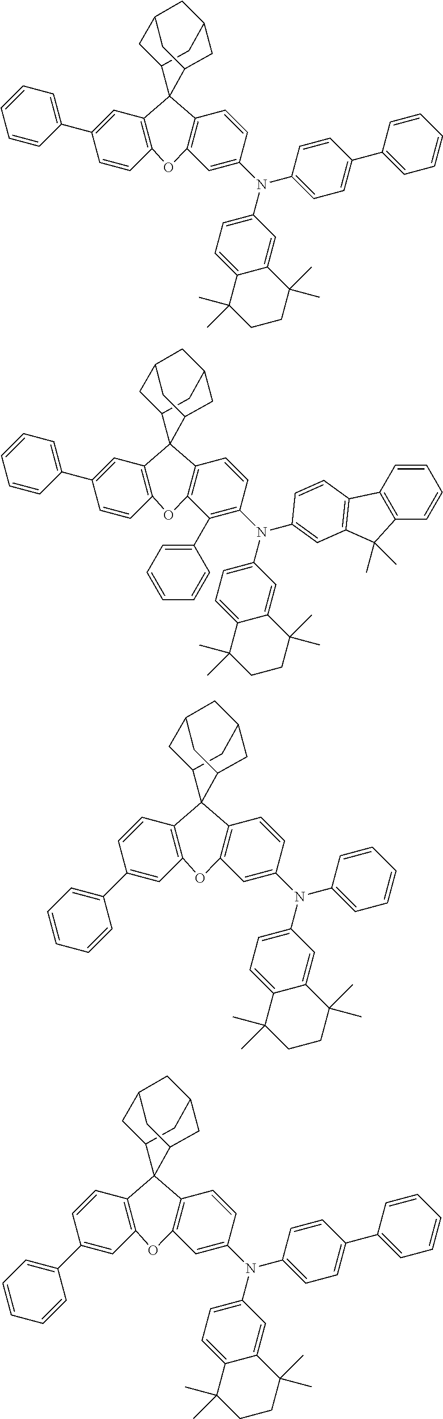

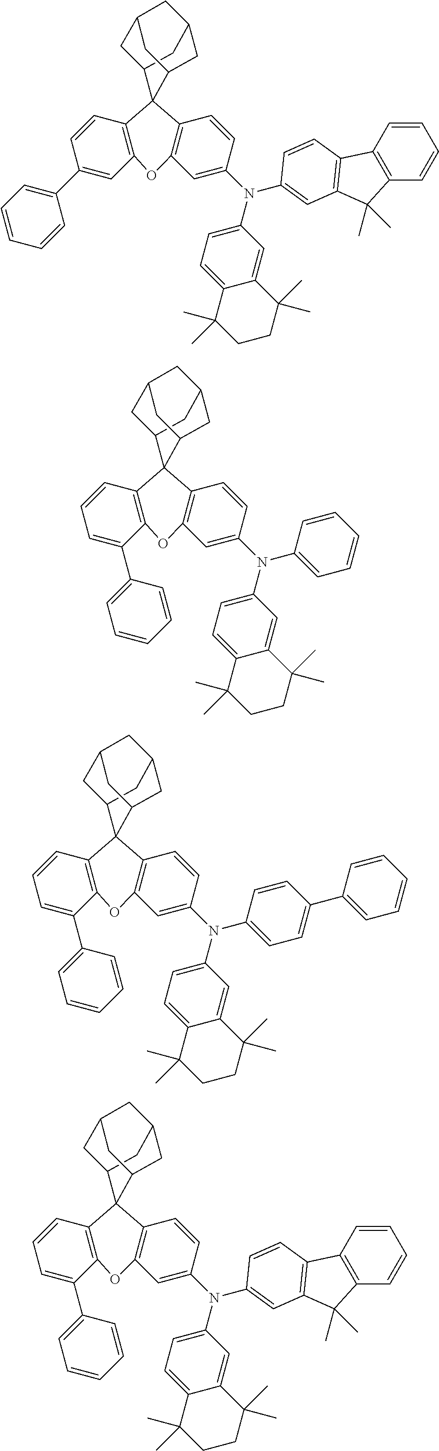

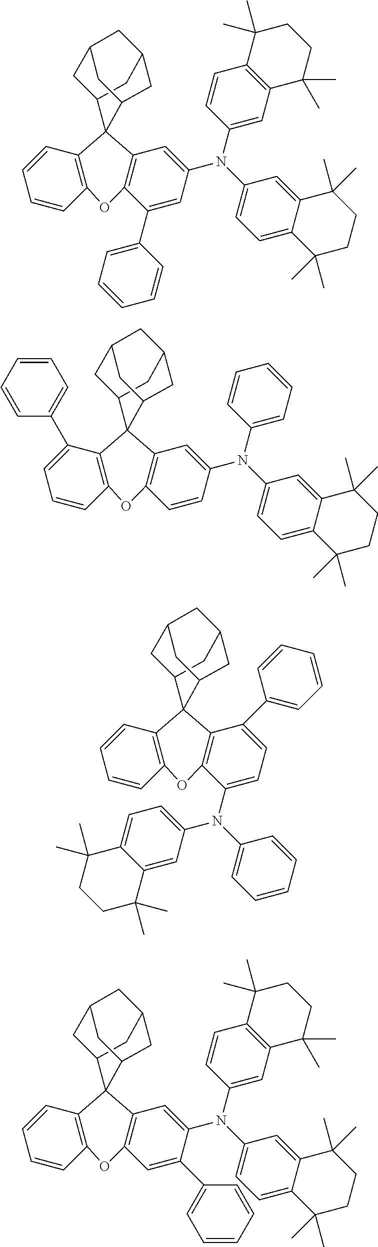

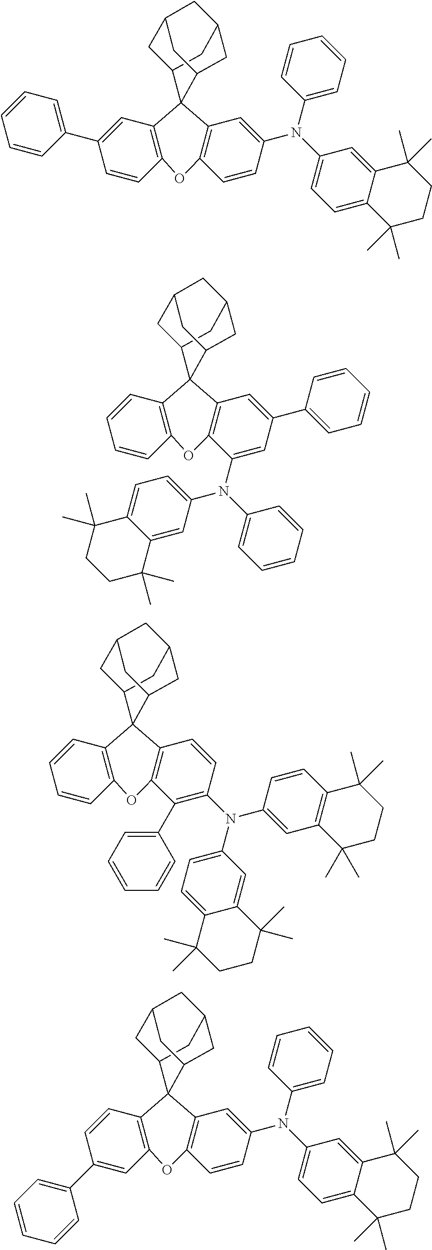

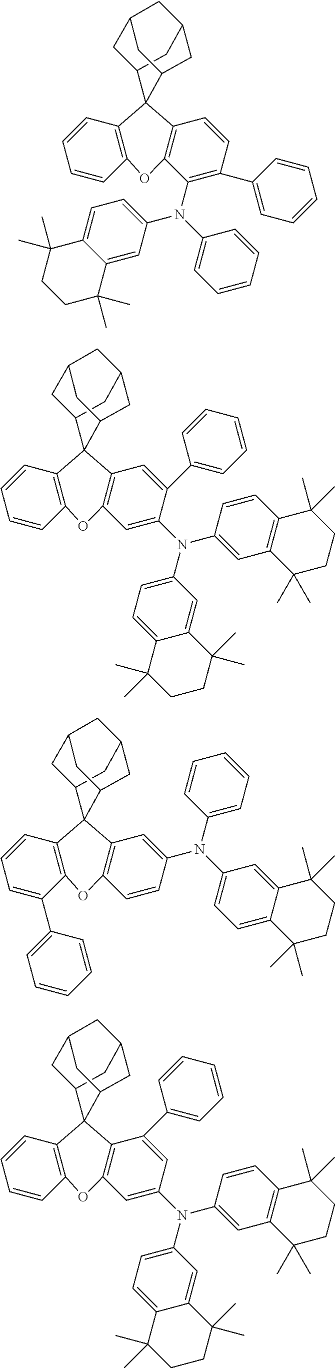

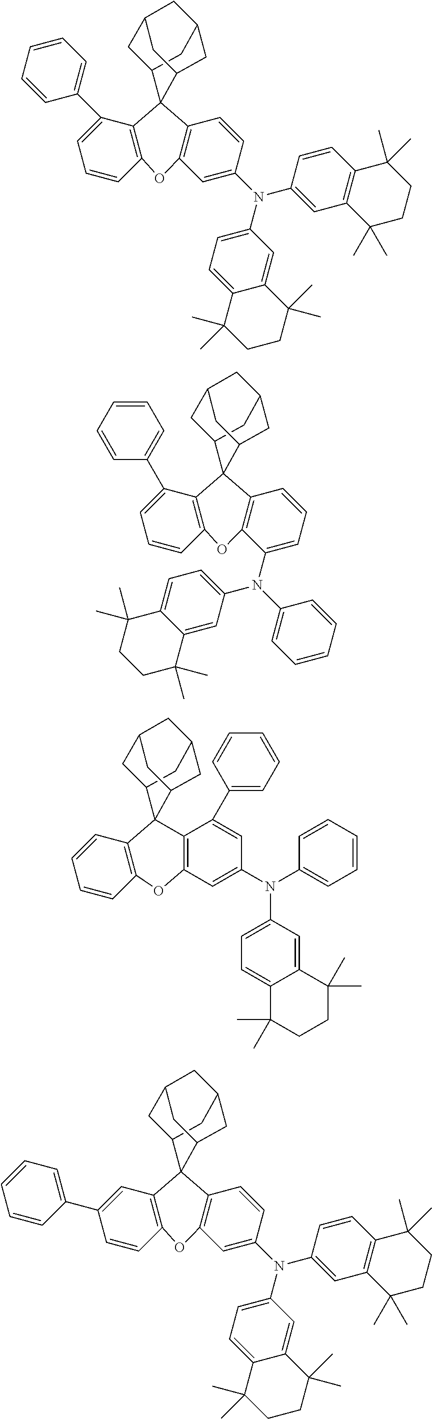

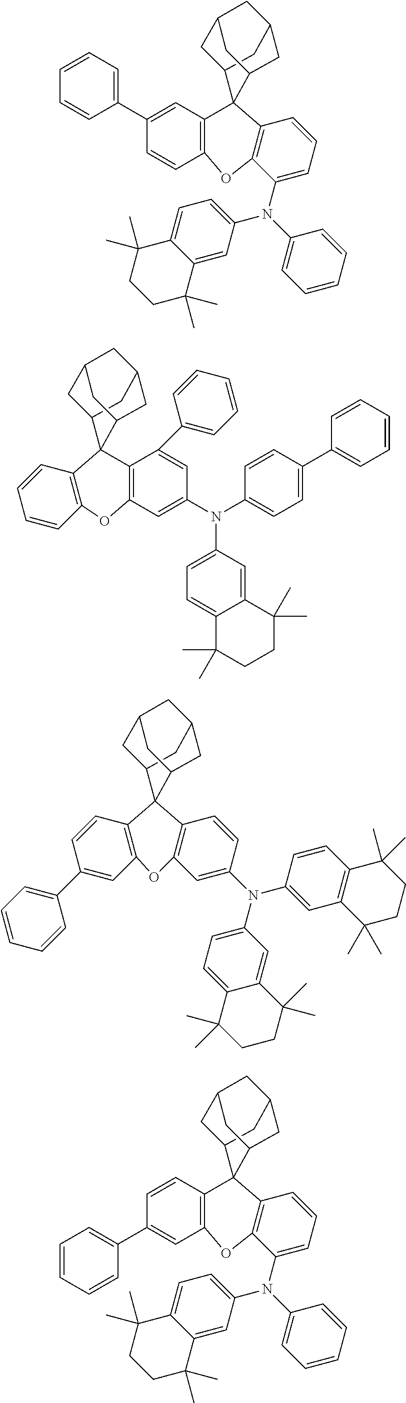

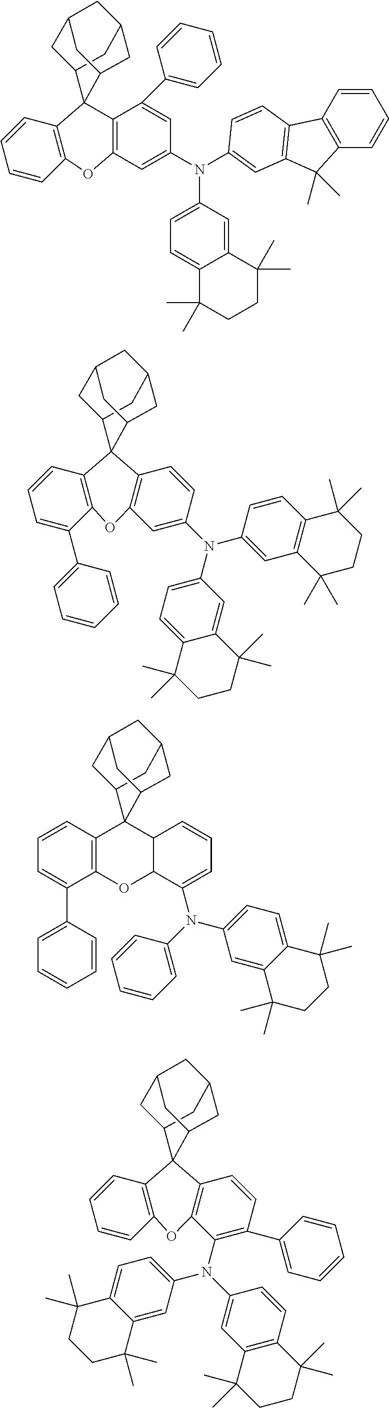

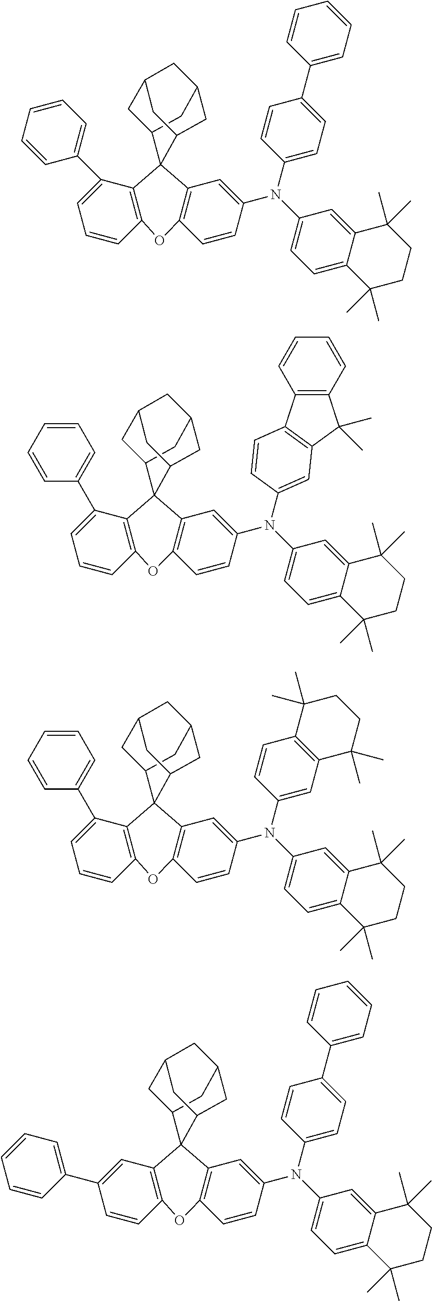

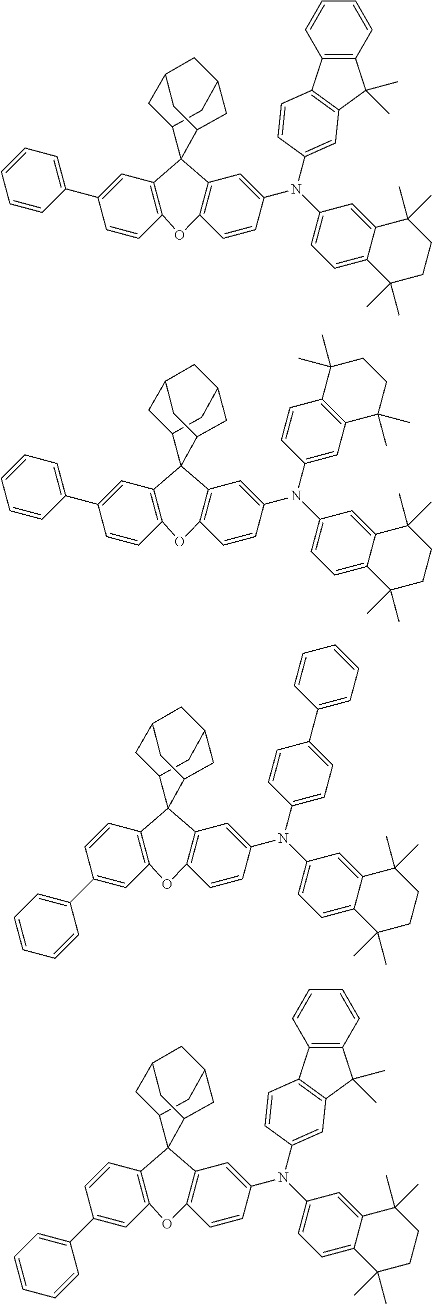

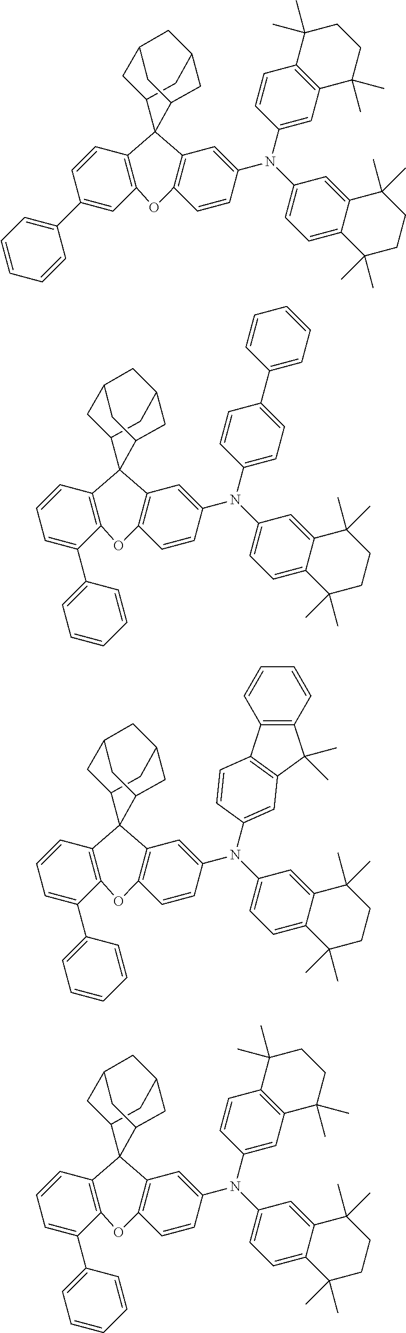 [SMILES: CC1(C)CCC(C)(C)c2cc(N(c3ccc4c(c3)C(C)(C)CCC4(C)C)c3ccc4c(c3)C3(c5ccc(-c6ccccc6)cc5O4)C4CC5CC(C4)CC3C5)ccc21 ; CC1(C)CCC(C)(C)c2cc(N(c3ccc(-c4ccccc4)cc3)c3ccc4c(c3)C3(c5cccc(-c6ccccc6)c5O4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5cccc(-c6ccccc6)c5O4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5cccc(-c6ccccc6)c5O4)C4CC5CC(C4)CC3C5)ccc21]

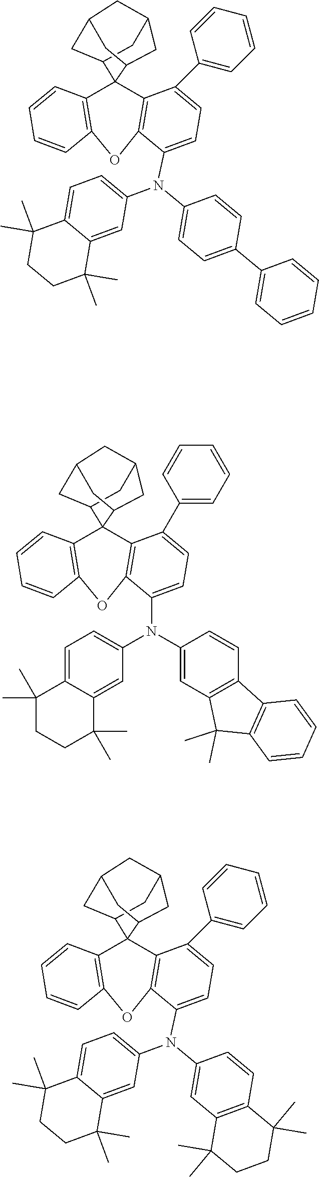

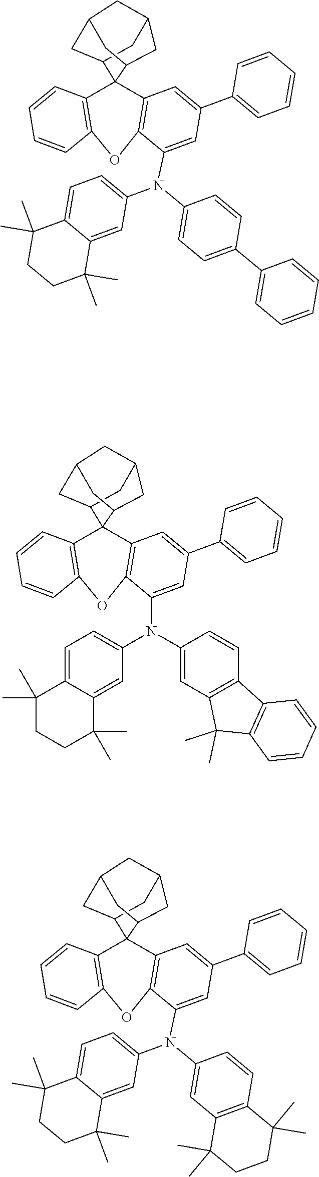 [SMILES: CC1(C)CCC(C)(C)c2cc(N(c3ccc(-c4ccccc4)cc3)c3cc(-c4ccccc4)cc4c3Oc3ccccc3C43C4CC5CC(C4)CC3C5)ccc21 ; CC1(C)CCC(C)(C)c2cc(N(c3ccc4c(c3)C(C)(C)c3ccccc3-4)c3cc(-c4ccccc4)cc4c3Oc3ccccc3C43C4CC5CC(C4)CC3C5)ccc21 ; CC1(C)CCC(C)(C)c2cc(N(c3ccc4c(c3)C(C)(C)CCC4(C)C)c3cc(-c4ccccc4)cc4c3Oc3ccccc3C43C4CC5CC(C4)CC3C5)ccc21]

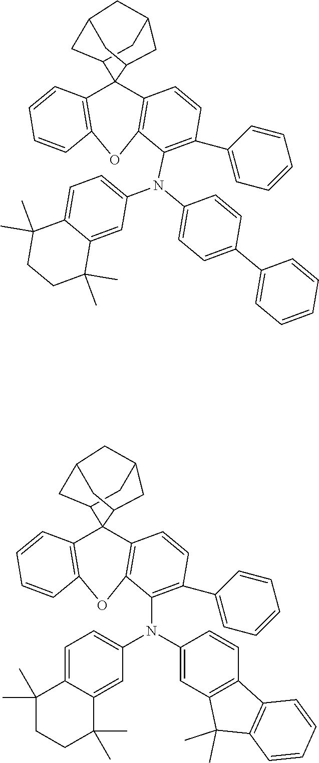 [SMILES: CC1(C)CCC(C)(C)c2cc(N(c3ccc(-c4ccccc4)cc3)c3c(-c4ccccc4)ccc4c3Oc3ccccc3C43C4CC5CC(C4)CC3C5)ccc21 ; CC1(C)CCC(C)(C)c2cc(N(c3ccc4c(c3)C(C)(C)c3ccccc3-4)c3c(-c4ccccc4)ccc4c3Oc3ccccc3C43C4CC5CC(C4)CC3C5)ccc21]

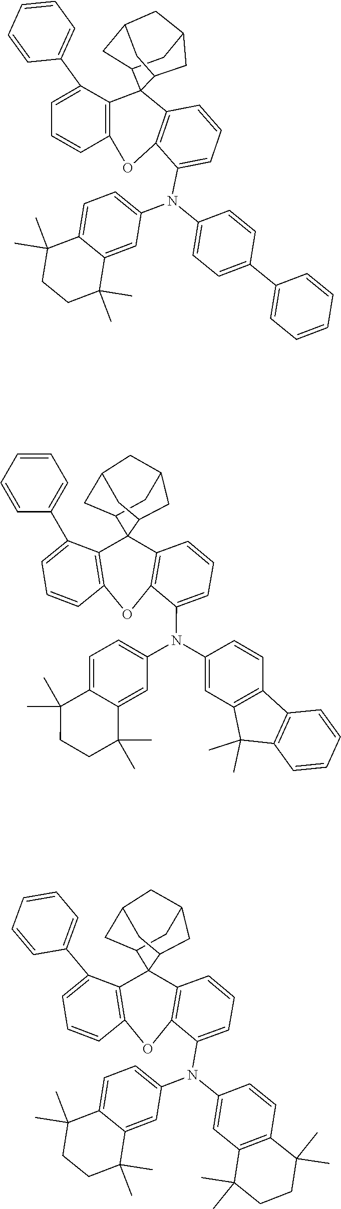 [SMILES: CC1(C)CCC(C)(C)c2cc(N(c3ccc(-c4ccccc4)cc3)c3cccc4c3Oc3cccc(-c5ccccc5)c3C43C4CC5CC(C4)CC3C5)ccc21 ; CC1(C)CCC(C)(C)c2cc(N(c3ccc4c(c3)C(C)(C)c3ccccc3-4)c3cccc4c3Oc3cccc(-c5ccccc5)c3C43C4CC5CC(C4)CC3C5)ccc21 ; CC1(C)CCC(C)(C)c2cc(N(c3ccc4c(c3)C(C)(C)CCC4(C)C)c3cccc4c3Oc3cccc(-c5ccccc5)c3C43C4CC5CC(C4)CC3C5)ccc21]

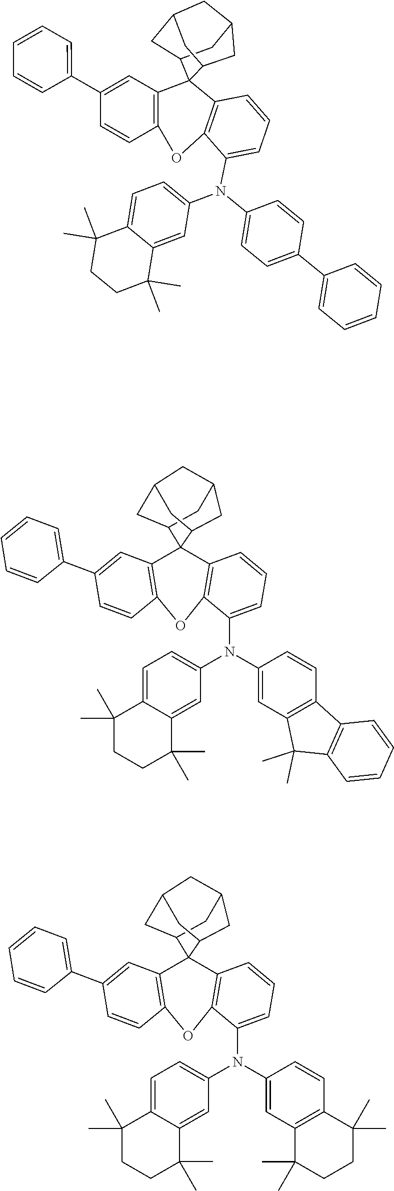 [SMILES: CC1(C)CCC(C)(C)c2cc(N(c3ccc(-c4ccccc4)cc3)c3cccc4c3Oc3ccc(-c5ccccc5)cc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3ccc(-c5ccccc5)cc3C43C4CC5CC(C4)CC3C5)ccc21 ; CC1(C)CCC(C)(C)c2cc(N(c3ccc4c(c3)C(C)(C)CCC4(C)C)c3cccc4c3Oc3ccc(-c5ccccc5)cc3C43C4CC5CC(C4)CC3C5)ccc21]

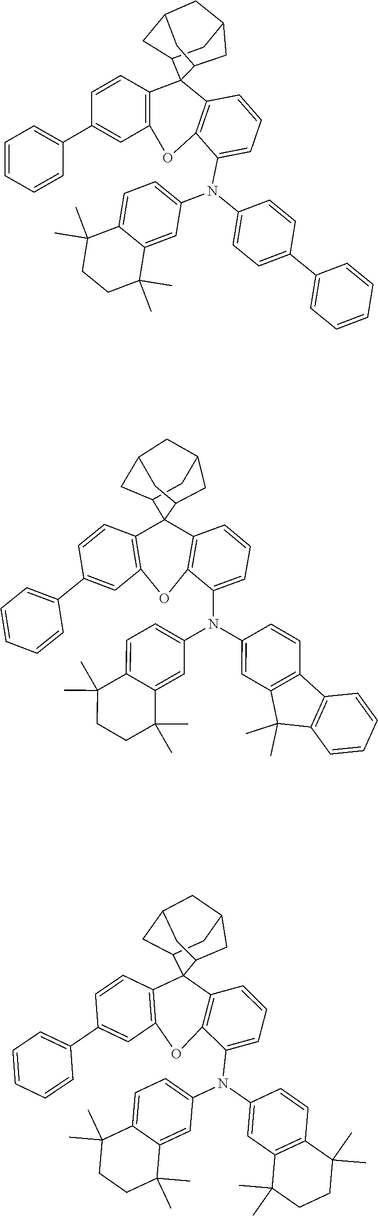 [SMILES: CC1(C)CCC(C)(C)c2cc(N(c3ccc(-c4ccccc4)cc3)c3cccc4c3Oc3cc(-c5ccccc5)ccc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3cc(-c5ccccc5)ccc3C43C4CC5CC(C4)CC3C5)ccc21 ; CC1(C)CCC(C)(C)c2cc(N(c3ccc4c(c3)C(C)(C)CCC4(C)C)c3cccc4c3Oc3cc(-c5ccccc5)ccc3C43C4CC5CC(C4)CC3C5)ccc21]

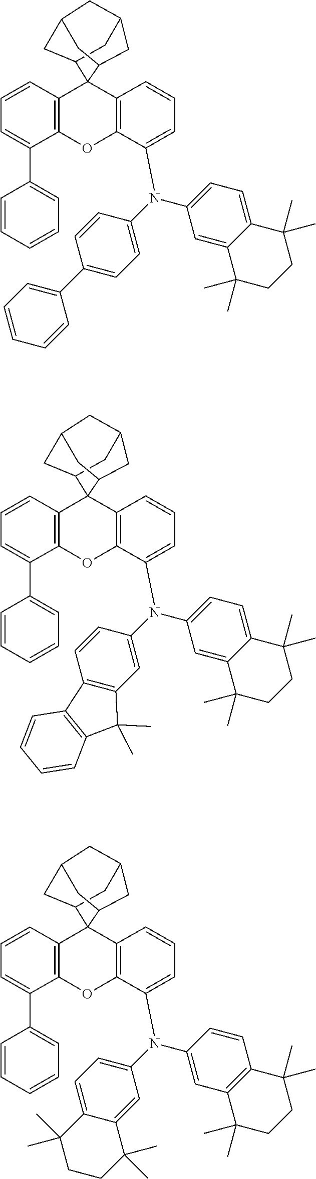 [SMILES: CC1(C)CCC(C)(C)c2cc(N(c3ccc(-c4ccccc4)cc3)c3cccc4c3Oc3c(-c5ccccc5)cccc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3c(-c5ccccc5)cccc3C43C4CC5CC(C4)CC3C5)ccc21 ; CC1(C)CCC(C)(C)c2cc(N(c3ccc4c(c3)C(C)(C)CCC4(C)C)c3cccc4c3Oc3c(-c5ccccc5)cccc3C43C4CC5CC(C4)CC3C5)ccc21]

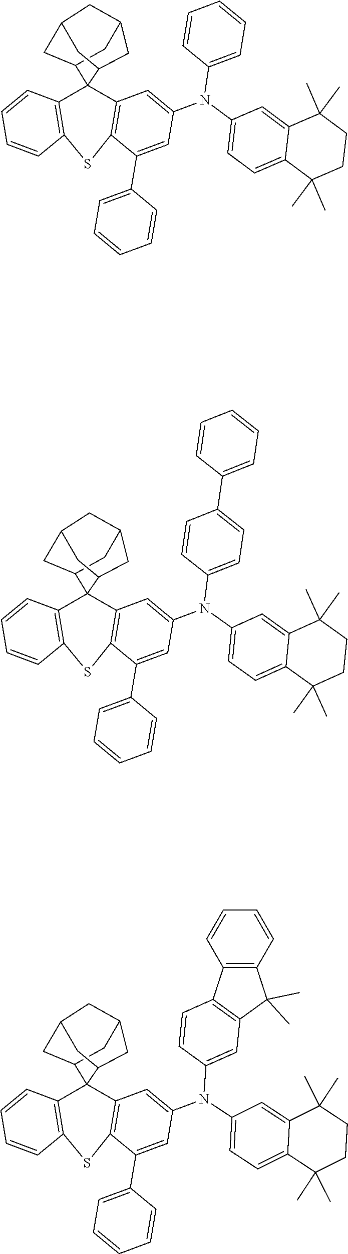 [SMILES: CC1(C)CCC(C)(C)c2cc(N(c3ccccc3)c3cc(-c4ccccc4)c4c(c3)C3(c5ccccc5S4)C4CC5CC(C4)CC3C5)ccc21 ; CC1(C)CCC(C)(C)c2cc(N(c3ccc(-c4ccccc4)cc3)c3cc(-c4ccccc4)c4c(c3)C3(c5ccccc5S4)C4CC5CC(C4)CC3C5)ccc21 ; CC1(C)CCC(C)(C)c2cc(N(c3ccc4c(c3)C(C)(C)c3ccccc3-4)c3cc(-c4ccccc4)c4c(c3)C3(c5ccccc5S4)C4CC5CC(C4)CC3C5)ccc21]

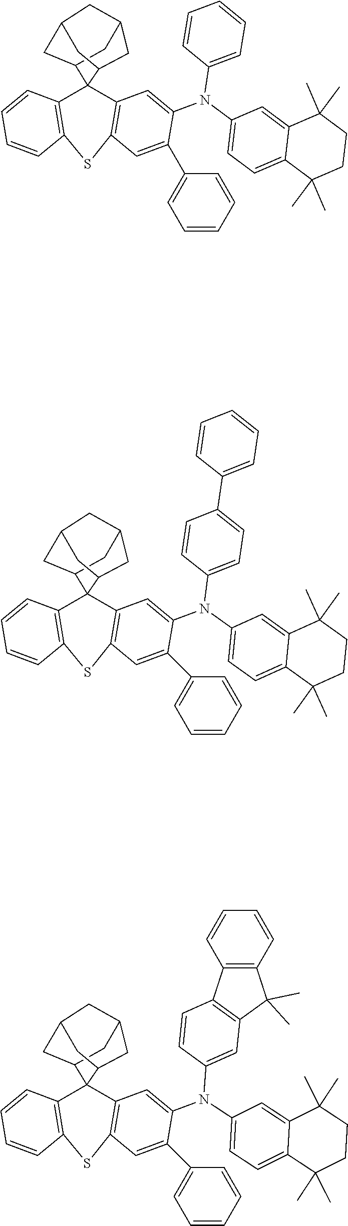 [SMILES: CC1(C)CCC(C)(C)c2cc(N(c3ccccc3)c3cc4c(cc3-c3ccccc3)Sc3ccccc3C43C4CC5CC(C4)CC3C5)ccc21 ; CC1(C)CCC(C)(C)c2cc(N(c3ccc(-c4ccccc4)cc3)c3cc4c(cc3-c3ccccc3)Sc3ccccc3C43C4CC5CC(C4)CC3C5)ccc21 ; CC1(C)CCC(C)(C)c2cc(N(c3ccc4c(c3)C(C)(C)c3ccccc3-4)c3cc4c(cc3-c3ccccc3)Sc3ccccc3C43C4CC5CC(C4)CC3C5)ccc21]

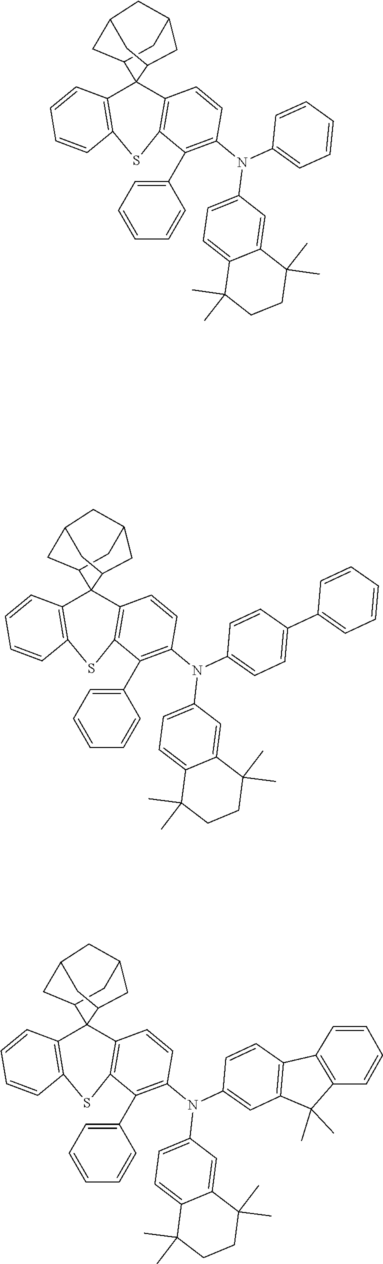 [SMILES: CC1(C)CCC(C)(C)c2cc(N(c3ccccc3)c3ccc4c(c3-c3ccccc3)Sc3ccccc3C43C4CC5CC(C4)CC3C5)ccc21 ; CC1(C)CCC(C)(C)c2cc(N(c3ccc(-c4ccccc4)cc3)c3ccc4c(c3-c3ccccc3)Sc3ccccc3C43C4CC5CC(C4)CC3C5)ccc21 ; CC1(C)CCC(C)(C)c2cc(N(c3ccc4c(c3)C(C)(C)c3ccccc3-4)c3ccc4c(c3-c3ccccc3)Sc3ccccc3C43C4CC5CC(C4)CC3C5)ccc21]

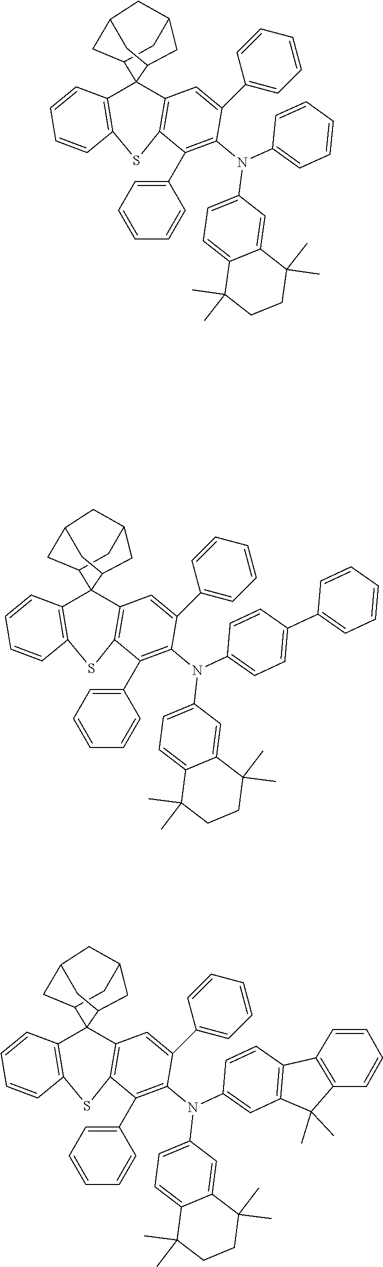 [SMILES: CC1(C)CCC(C)(C)c2cc(N(c3ccccc3)c3c(-c4ccccc4)cc4c(c3-c3ccccc3)Sc3ccccc3C43C4CC5CC(C4)CC3C5)ccc21 ; CC1(C)CCC(C)(C)c2cc(N(c3ccc(-c4ccccc4)cc3)c3c(-c4ccccc4)cc4c(c3-c3ccccc3)Sc3ccccc3C43C4CC5CC(C4)CC3C5)ccc21 ; CC1(C)CCC(C)(C)c2cc(N(c3ccc4c(c3)C(C)(C)c3ccccc3-4)c3c(-c4ccccc4)cc4c(c3-c3ccccc3)Sc3ccccc3C43C4CC5CC(C4)CC3C5)ccc21]

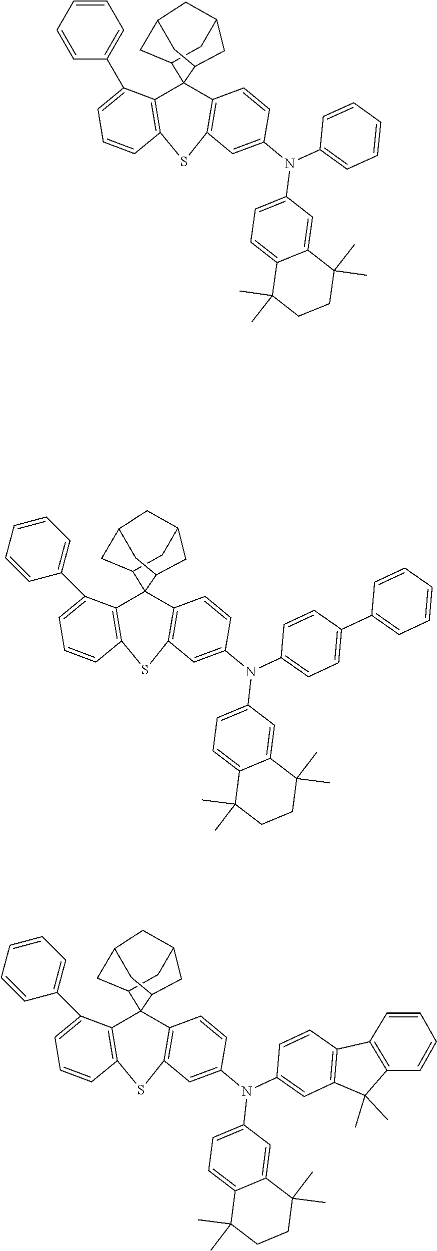 [SMILES: CC1(C)CCC(C)(C)c2cc(N(c3ccccc3)c3ccc4c(c3)Sc3cccc(-c5ccccc5)c3C43C4CC5CC(C4)CC3C5)ccc21 ; CC1(C)CCC(C)(C)c2cc(N(c3ccc(-c4ccccc4)cc3)c3ccc4c(c3)Sc3cccc(-c5ccccc5)c3C43C4CC5CC(C4)CC3C5)ccc21 ; CC1(C)CCC(C)(C)c2cc(N(c3ccc4c(c3)Sc3cccc(-c5ccccc5)c3C43C4CC5CC(C4)CC3C5)c3ccc4c(c3)C(C)(C)c3ccccc3-4)ccc21]

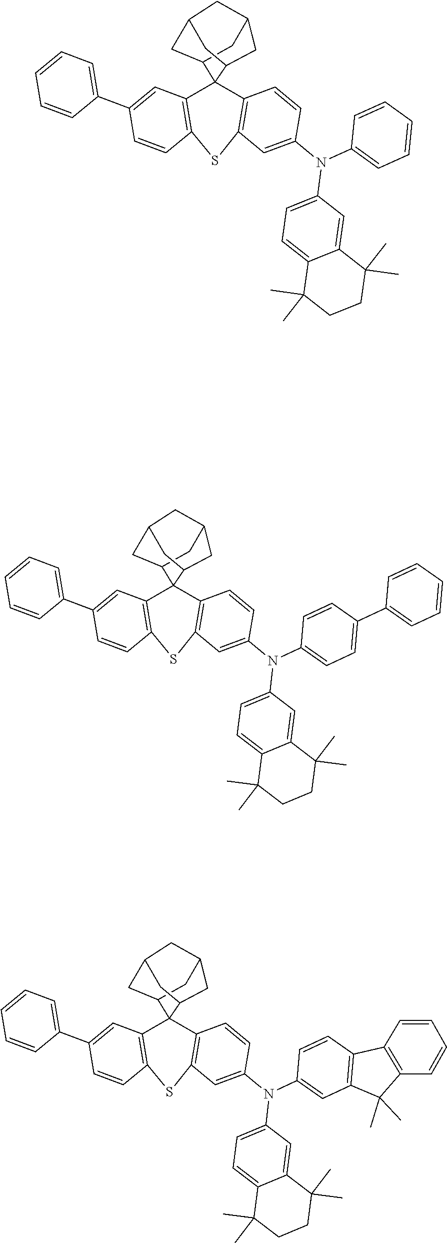 [SMILES: CC1(C)CCC(C)(C)c2cc(N(c3ccccc3)c3ccc4c(c3)Sc3ccc(-c5ccccc5)cc3C43C4CC5CC(C4)CC3C5)ccc21 ; CC1(C)CCC(C)(C)c2cc(N(c3ccc(-c4ccccc4)cc3)c3ccc4c(c3)Sc3ccc(-c5ccccc5)cc3C43C4CC5CC(C4)CC3C5)ccc21 ; CC1(C)CCC(C)(C)c2cc(N(c3ccc4c(c3)Sc3ccc(-c5ccccc5)cc3C43C4CC5CC(C4)CC3C5)c3ccc4c(c3)C(C)(C)c3ccccc3-4)ccc21]

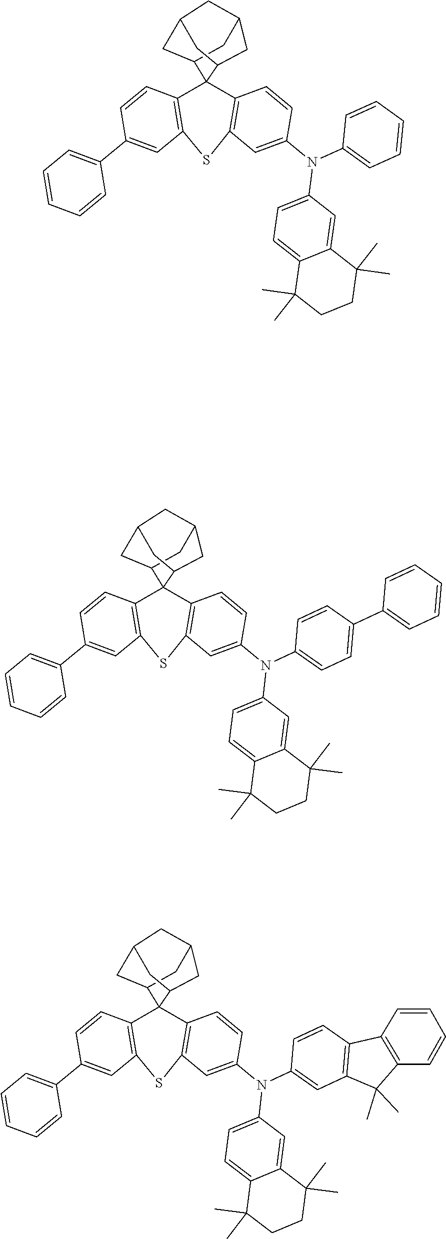 [SMILES: CC1(C)CCC(C)(C)c2cc(N(c3ccccc3)c3ccc4c(c3)Sc3cc(-c5ccccc5)ccc3C43C4CC5CC(C4)CC3C5)ccc21 ; CC1(C)CCC(C)(C)c2cc(N(c3ccc(-c4ccccc4)cc3)c3ccc4c(c3)Sc3cc(-c5ccccc5)ccc3C43C4CC5CC(C4)CC3C5)ccc21 ; CC1(C)CCC(C)(C)c2cc(N(c3ccc4c(c3)Sc3cc(-c5ccccc5)ccc3C43C4CC5CC(C4)CC3C5)c3ccc4c(c3)C(C)(C)c3ccccc3-4)ccc21]

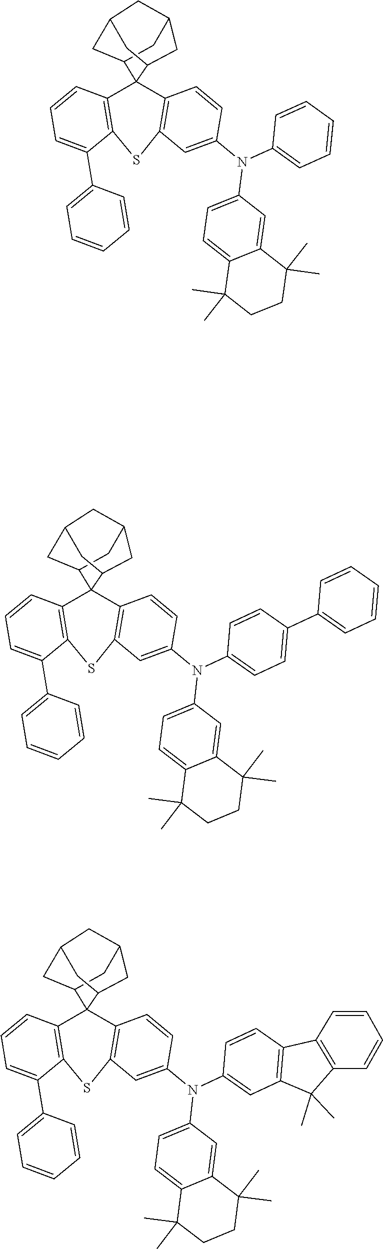 [SMILES: CC1(C)CCC(C)(C)c2cc(N(c3ccccc3)c3ccc4c(c3)Sc3c(-c5ccccc5)cccc3C43C4CC5CC(C4)CC3C5)ccc21 ; CC1(C)CCC(C)(C)c2cc(N(c3ccc(-c4ccccc4)cc3)c3ccc4c(c3)Sc3c(-c5ccccc5)cccc3C43C4CC5CC(C4)CC3C5)ccc21 ; CC1(C)CCC(C)(C)c2cc(N(c3ccc4c(c3)Sc3c(-c5ccccc5)cccc3C43C4CC5CC(C4)CC3C5)c3ccc4c(c3)C(C)(C)c3ccccc3-4)ccc21]

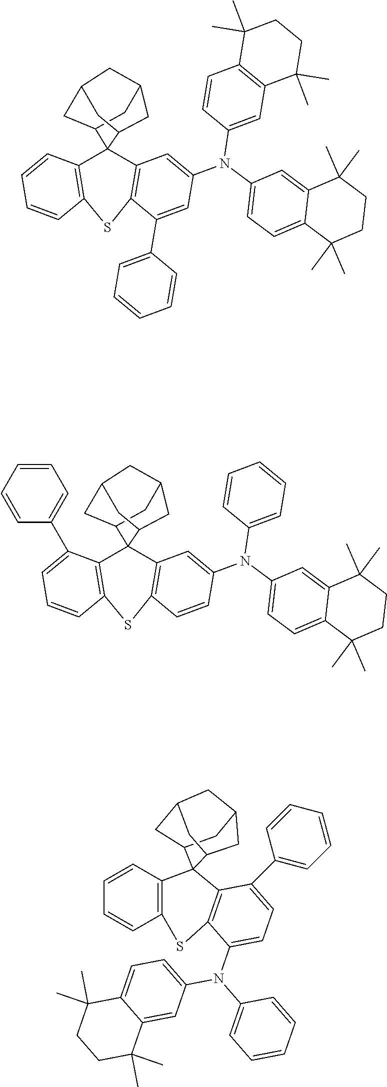 [SMILES: CC1(C)CCC(C)(C)c2cc(N(c3ccc4c(c3)C(C)(C)CCC4(C)C)c3cc(-c4ccccc4)c4c(c3)C3(c5ccccc5S4)C4CC5CC(C4)CC3C5)ccc21 ; CC1(C)CCC(C)(C)c2cc(N(c3ccccc3)c3ccc4c(c3)C3(c5c(cccc5-c5ccccc5)S4)C4CC5CC(C4)CC3C5)ccc21 ; CC1(C)CCC(C)(C)c2cc(N(c3ccccc3)c3ccc(-c4ccccc4)c4c3Sc3ccccc3C43C4CC5CC(C4)CC3C5)ccc21]

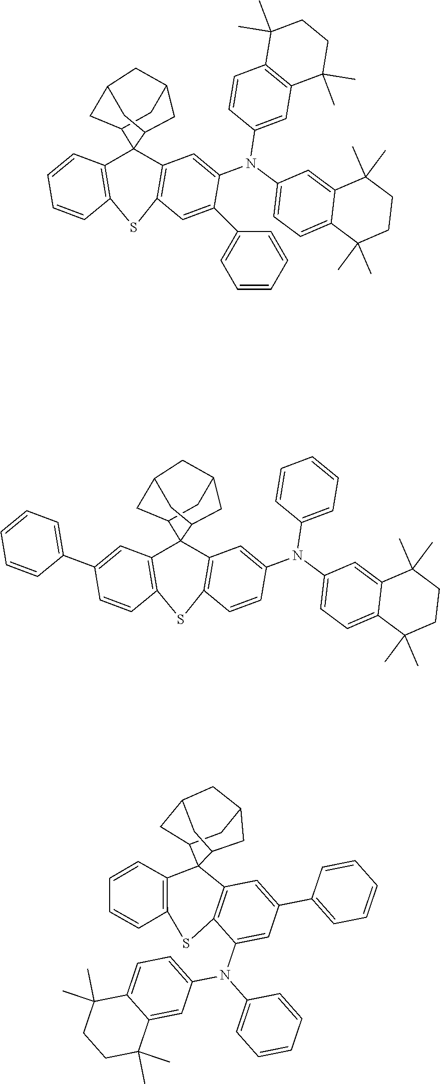 [SMILES: CC1(C)CCC(C)(C)c2cc(N(c3ccc4c(c3)C(C)(C)CCC4(C)C)c3cc4c(cc3-c3ccccc3)Sc3ccccc3C43C4CC5CC(C4)CC3C5)ccc21 ; CC1(C)CCC(C)(C)c2cc(N(c3ccccc3)c3ccc4c(c3)C3(c5cc(-c6ccccc6)ccc5S4)C4CC5CC(C4)CC3C5)ccc21 ; CC1(C)CCC(C)(C)c2cc(N(c3ccccc3)c3cc(-c4ccccc4)cc4c3Sc3ccccc3C43C4CC5CC(C4)CC3C5)ccc21]

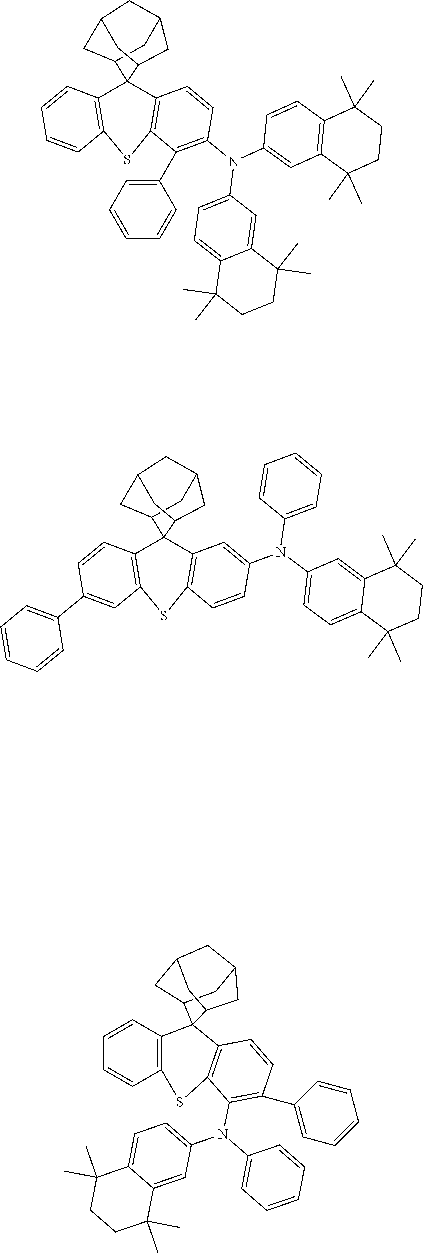 [SMILES: CC1(C)CCC(C)(C)c2cc(N(c3ccc4c(c3)C(C)(C)CCC4(C)C)c3ccc4c(c3-c3ccccc3)Sc3ccccc3C43C4CC5CC(C4)CC3C5)ccc21 ; CC1(C)CCC(C)(C)c2cc(N(c3ccccc3)c3ccc4c(c3)C3(c5ccc(-c6ccccc6)cc5S4)C4CC5CC(C4)CC3C5)ccc21 ; CC1(C)CCC(C)(C)c2cc(N(c3ccccc3)c3c(-c4ccccc4)ccc4c3Sc3ccccc3C43C4CC5CC(C4)CC3C5)ccc21]

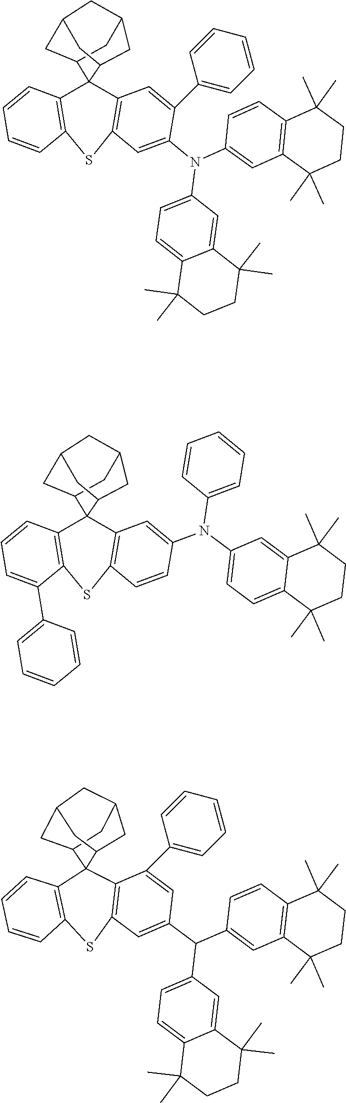 [SMILES: CC1(C)CCC(C)(C)c2cc(N(c3ccc4c(c3)C(C)(C)CCC4(C)C)c3cc4c(cc3-c3ccccc3)C3(c5ccccc5S4)C4CC5CC(C4)CC3C5)ccc21 ; CC1(C)CCC(C)(C)c2cc(N(c3ccccc3)c3ccc4c(c3)C3(c5cccc(-c6ccccc6)c5S4)C4CC5CC(C4)CC3C5)ccc21 ; CC1(C)CCC(C)(C)c2cc(C(c3cc4c(c(-c5ccccc5)c3)C3(c5ccccc5S4)C4CC5CC(C4)CC3C5)c3ccc4c(c3)C(C)(C)CCC4(C)C)ccc21]

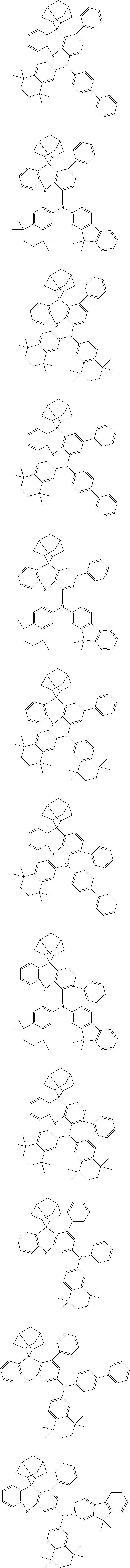

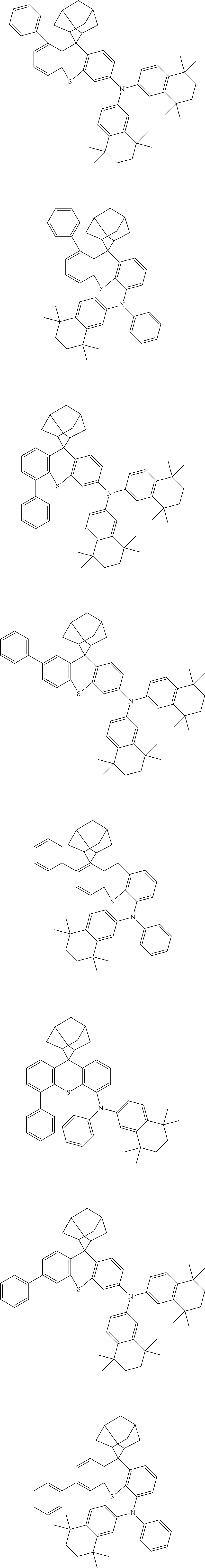

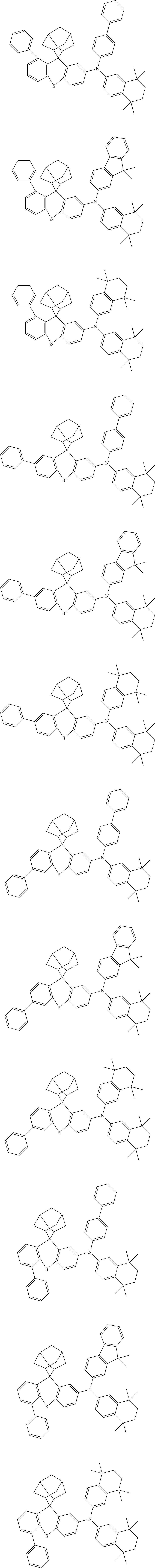

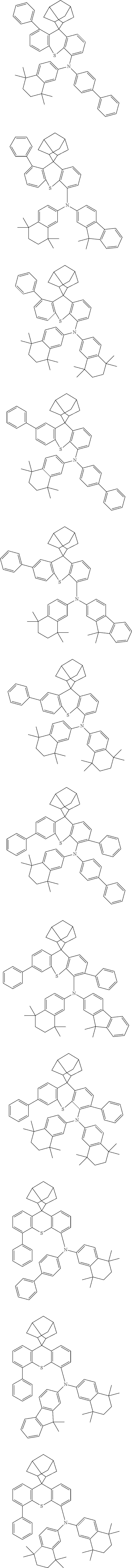

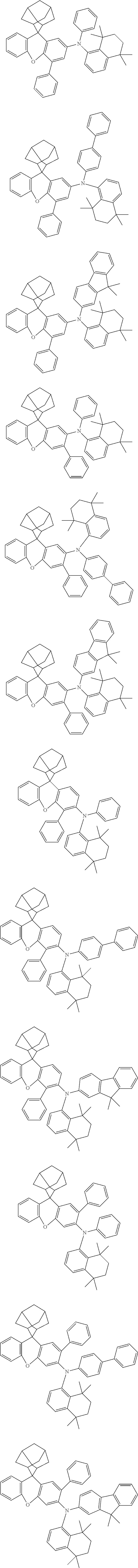

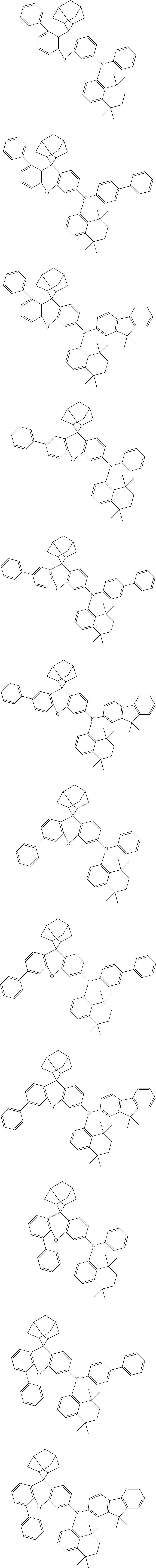

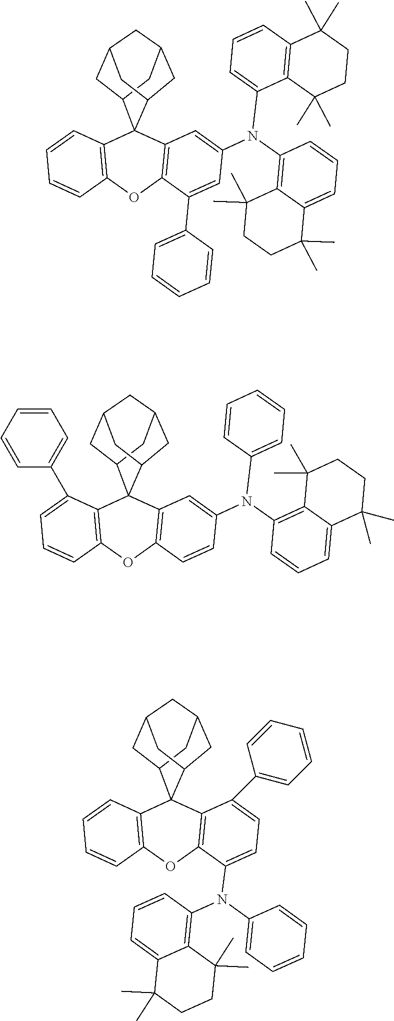 [SMILES: CC1(C)CCC(C)(C)c2c(N(c3cc(-c4ccccc4)c4c(c3)C3(c5ccccc5O4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5c(cccc5-c5ccccc5)O4)C4CC5CC(C4)CC3C5)cccc21 ; CC1(C)CCC(C)(C)c2c(N(c3ccccc3)c3ccc(-c4ccccc4)c4c3Oc3ccccc3C43C4CC5CC(C4)CC3C5)cccc21]

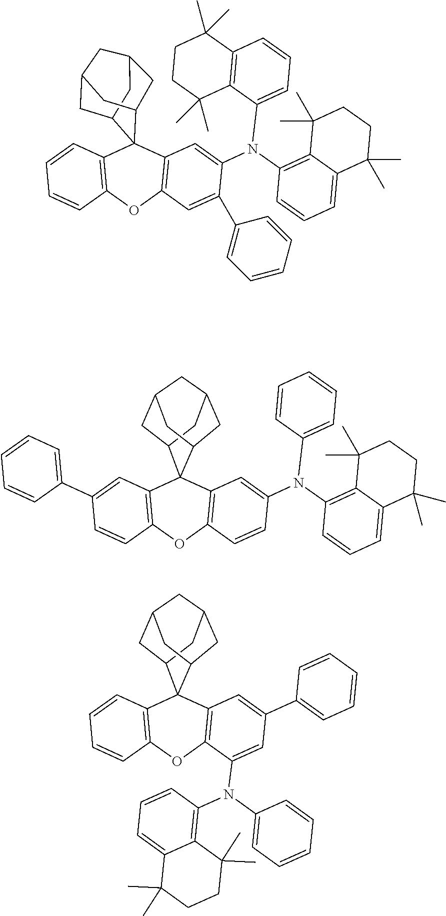 [SMILES: CC1(C)CCC(C)(C)c2c(N(c3cc4c(cc3-c3ccccc3)Oc3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cc(-c6ccccc6)ccc5O4)C4CC5CC(C4)CC3C5)cccc21 ; CC1(C)CCC(C)(C)c2c(N(c3ccccc3)c3cc(-c4ccccc4)cc4c3Oc3ccccc3C43C4CC5CC(C4)CC3C5)cccc21]

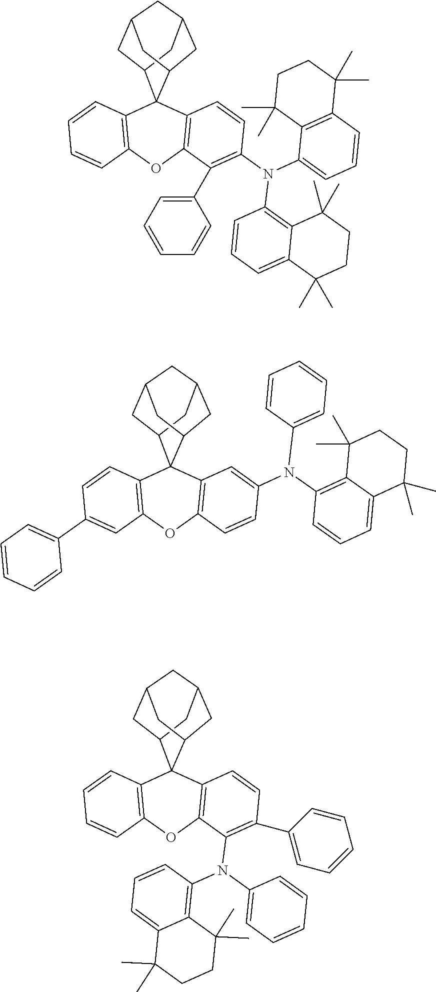 [SMILES: CC1(C)CCC(C)(C)c2c(N(c3ccc4c(c3-c3ccccc3)Oc3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5ccc(-c6ccccc6)cc5O4)C4CC5CC(C4)CC3C5)cccc21 ; CC1(C)CCC(C)(C)c2c(N(c3ccccc3)c3c(-c4ccccc4)ccc4c3Oc3ccccc3C43C4CC5CC(C4)CC3C5)cccc21]

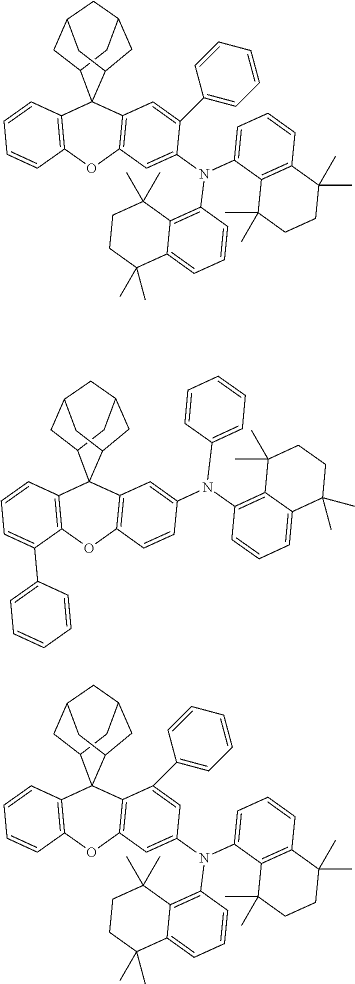 [SMILES: CC1(C)CCC(C)(C)c2c(N(c3cc4c(cc3-c3ccccc3)C3(c5ccccc5O4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cccc(-c6ccccc6)c5O4)C4CC5CC(C4)CC3C5)cccc21 ; CC1(C)CCC(C)(C)c2c(N(c3cc4c(c(-c5ccccc5)c3)C3(c5ccccc5O4)C4CC5CC(C4)CC3C5)c3cccc4c3C(C)(C)CCC4(C)C)cccc21]

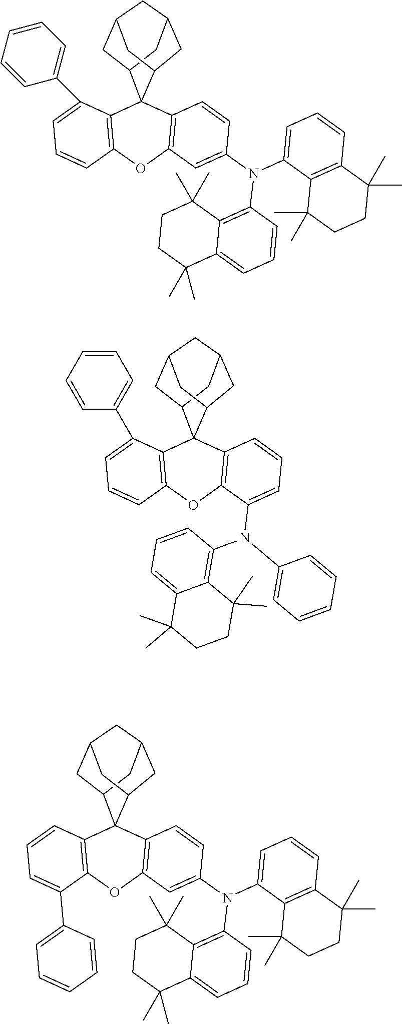

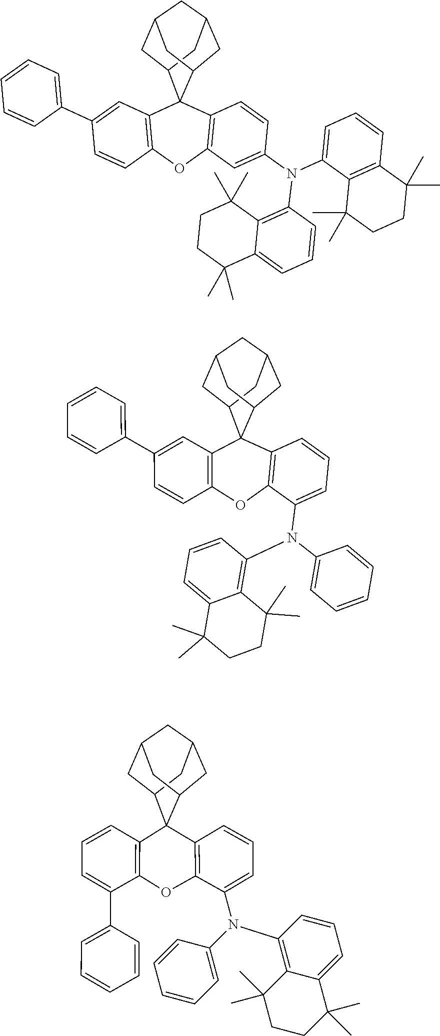

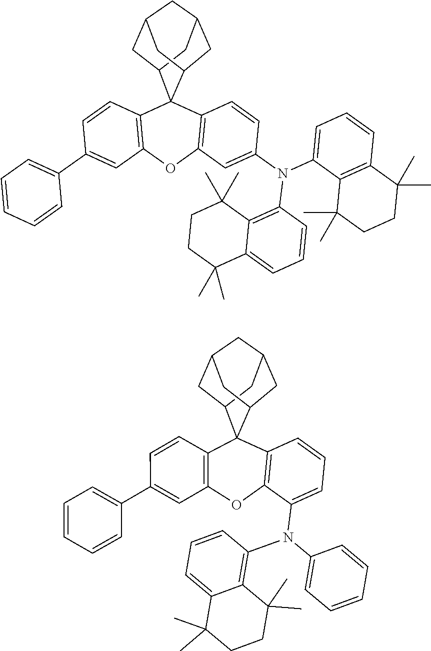

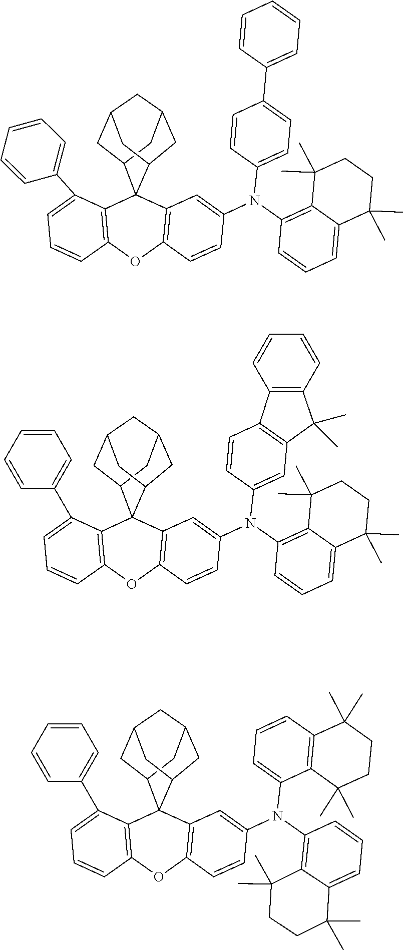 [SMILES: CC1(C)CCC(C)(C)c2c(N(c3ccc(-c4ccccc4)cc3)c3ccc4c(c3)C3(c5c(cccc5-c5ccccc5)O4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5c(cccc5-c5ccccc5)O4)C4CC5CC(C4)CC3C5)cccc21 ; CC1(C)CCC(C)(C)c2c(N(c3ccc4c(c3)C3(c5c(cccc5-c5ccccc5)O4)C4CC5CC(C4)CC3C5)c3cccc4c3C(C)(C)CCC4(C)C)cccc21]

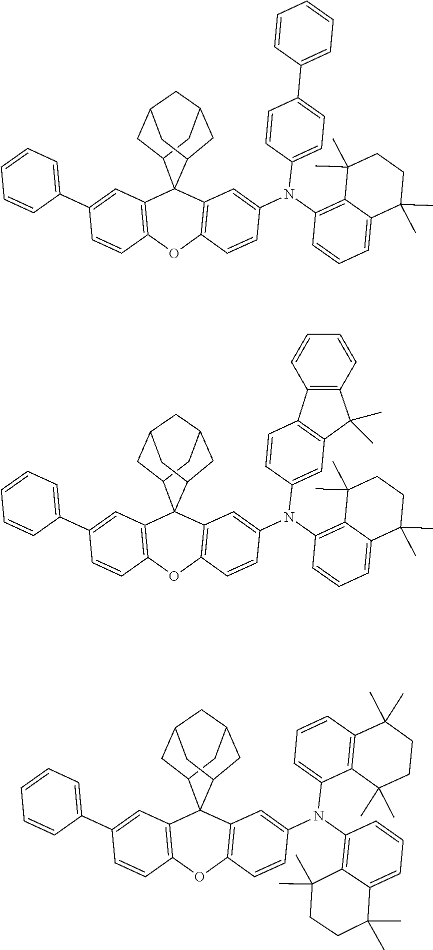 [SMILES: CC1(C)CCC(C)(C)c2c(N(c3ccc(-c4ccccc4)cc3)c3ccc4c(c3)C3(c5cc(-c6ccccc6)ccc5O4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cc(-c6ccccc6)ccc5O4)C4CC5CC(C4)CC3C5)cccc21 ; CC1(C)CCC(C)(C)c2c(N(c3ccc4c(c3)C3(c5cc(-c6ccccc6)ccc5O4)C4CC5CC(C4)CC3C5)c3cccc4c3C(C)(C)CCC4(C)C)cccc21]

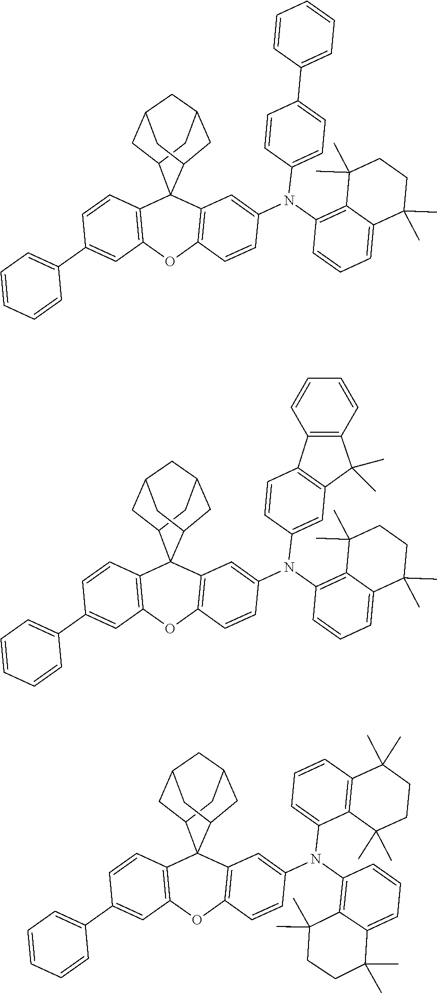 [SMILES: CC1(C)CCC(C)(C)c2c(N(c3ccc(-c4ccccc4)cc3)c3ccc4c(c3)C3(c5ccc(-c6ccccc6)cc5O4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5ccc(-c6ccccc6)cc5O4)C4CC5CC(C4)CC3C5)cccc21 ; CC1(C)CCC(C)(C)c2c(N(c3ccc4c(c3)C3(c5ccc(-c6ccccc6)cc5O4)C4CC5CC(C4)CC3C5)c3cccc4c3C(C)(C)CCC4(C)C)cccc21]

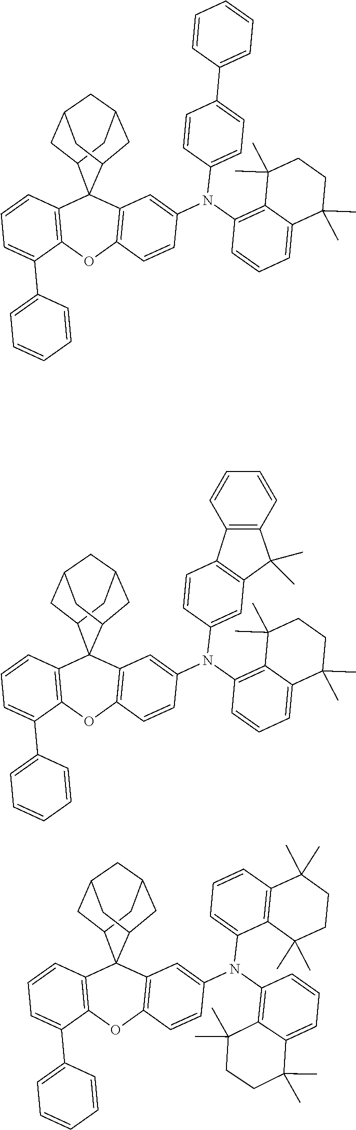 [SMILES: CC1(C)CCC(C)(C)c2c(N(c3ccc(-c4ccccc4)cc3)c3ccc4c(c3)C3(c5cccc(-c6ccccc6)c5O4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cccc(-c6ccccc6)c5O4)C4CC5CC(C4)CC3C5)cccc21 ; CC1(C)CCC(C)(C)c2c(N(c3ccc4c(c3)C3(c5cccc(-c6ccccc6)c5O4)C4CC5CC(C4)CC3C5)c3cccc4c3C(C)(C)CCC4(C)C)cccc21]

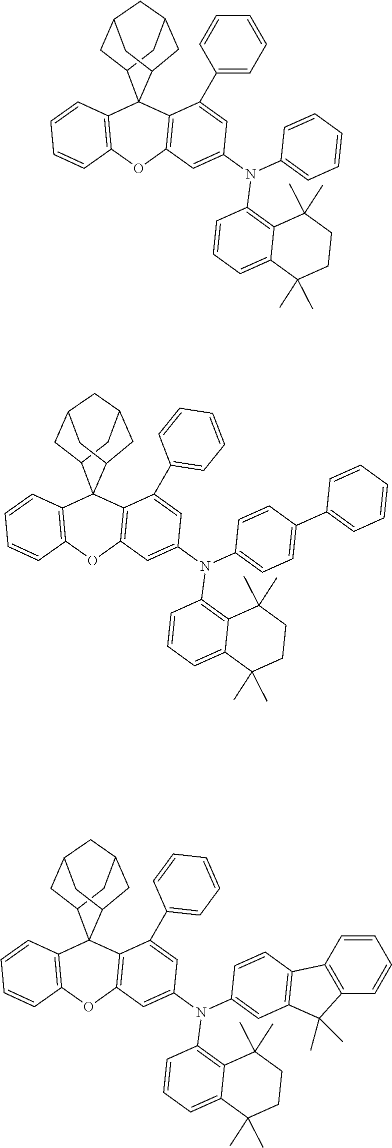 [SMILES: CC1(C)CCC(C)(C)c2c(N(c3ccccc3)c3cc4c(c(-c5ccccc5)c3)C3(c5ccccc5O4)C4CC5CC(C4)CC3C5)cccc21 ; CC1(C)CCC(C)(C)c2c(N(c3ccc(-c4ccccc4)cc3)c3cc4c(c(-c5ccccc5)c3)C3(c5ccccc5O4)C4CC5CC(C4)CC3C5)cccc21 ; CC1(C)CCC(C)(C)c2c(N(c3cc4c(c(-c5ccccc5)c3)C3(c5ccccc5O4)C4CC5CC(C4)CC3C5)c3ccc4c(c3)C(C)(C)c3ccccc3-4)cccc21]

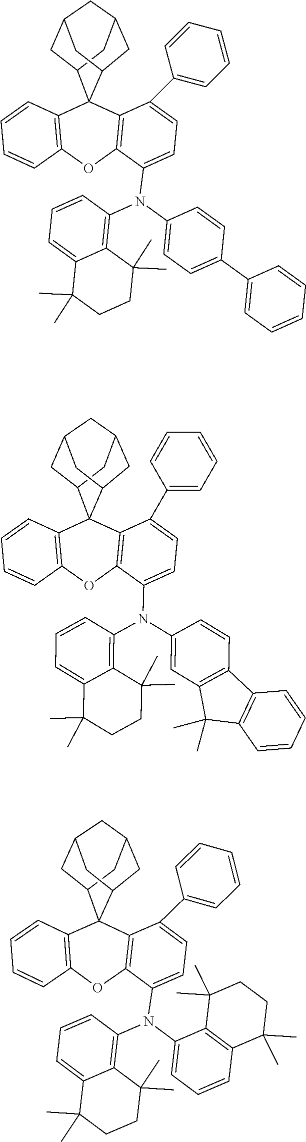 [SMILES: CC1(C)CCC(C)(C)c2c(N(c3ccc(-c4ccccc4)cc3)c3ccc(-c4ccccc4)c4c3Oc3ccccc3C43C4CC5CC(C4)CC3C5)cccc21 ; CC1(C)CCC(C)(C)c2c(N(c3ccc4c(c3)C(C)(C)c3ccccc3-4)c3ccc(-c4ccccc4)c4c3Oc3ccccc3C43C4CC5CC(C4)CC3C5)cccc21 ; CC1(C)CCC(C)(C)c2c(N(c3ccc(-c4ccccc4)c4c3Oc3ccccc3C43C4CC5CC(C4)CC3C5)c3cccc4c3C(C)(C)CCC4(C)C)cccc21]

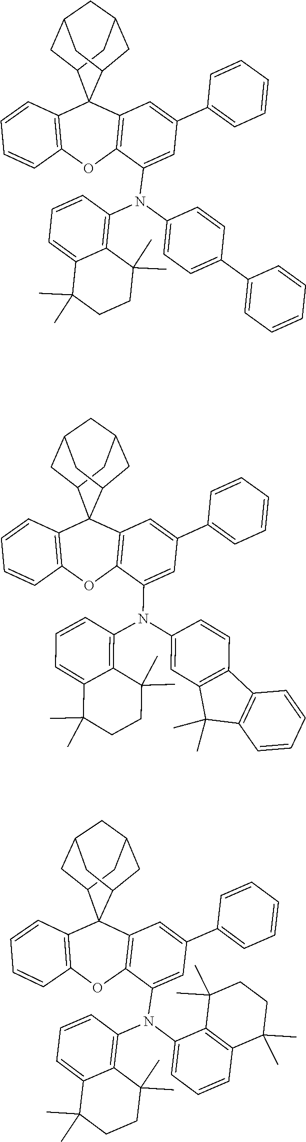 [SMILES: CC1(C)CCC(C)(C)c2c(N(c3ccc(-c4ccccc4)cc3)c3cc(-c4ccccc4)cc4c3Oc3ccccc3C43C4CC5CC(C4)CC3C5)cccc21 ; CC1(C)CCC(C)(C)c2c(N(c3ccc4c(c3)C(C)(C)c3ccccc3-4)c3cc(-c4ccccc4)cc4c3Oc3ccccc3C43C4CC5CC(C4)CC3C5)cccc21 ; CC1(C)CCC(C)(C)c2c(N(c3cc(-c4ccccc4)cc4c3Oc3ccccc3C43C4CC5CC(C4)CC3C5)c3cccc4c3C(C)(C)CCC4(C)C)cccc21]

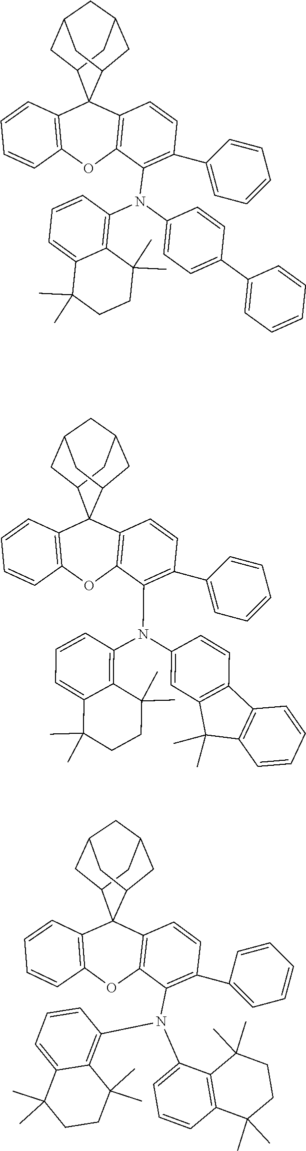 [SMILES: CC1(C)CCC(C)(C)c2c(N(c3ccc(-c4ccccc4)cc3)c3c(-c4ccccc4)ccc4c3Oc3ccccc3C43C4CC5CC(C4)CC3C5)cccc21 ; CC1(C)CCC(C)(C)c2c(N(c3ccc4c(c3)C(C)(C)c3ccccc3-4)c3c(-c4ccccc4)ccc4c3Oc3ccccc3C43C4CC5CC(C4)CC3C5)cccc21 ; CC1(C)CCC(C)(C)c2c(N(c3cccc4c3C(C)(C)CCC4(C)C)c3c(-c4ccccc4)ccc4c3Oc3ccccc3C43C4CC5CC(C4)CC3C5)cccc21]

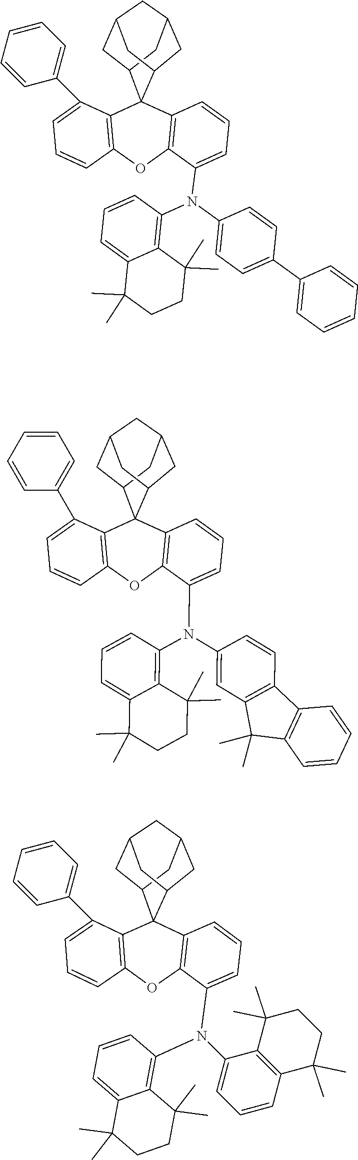 [SMILES: CC1(C)CCC(C)(C)c2c(N(c3ccc(-c4ccccc4)cc3)c3cccc4c3Oc3cccc(-c5ccccc5)c3C43C4CC5CC(C4)CC3C5)cccc21 ; CC1(C)CCC(C)(C)c2c(N(c3ccc4c(c3)C(C)(C)c3ccccc3-4)c3cccc4c3Oc3cccc(-c5ccccc5)c3C43C4CC5CC(C4)CC3C5)cccc21 ; CC1(C)CCC(C)(C)c2c(N(c3cccc4c3Oc3cccc(-c5ccccc5)c3C43C4CC5CC(C4)CC3C5)c3cccc4c3C(C)(C)CCC4(C)C)cccc21]

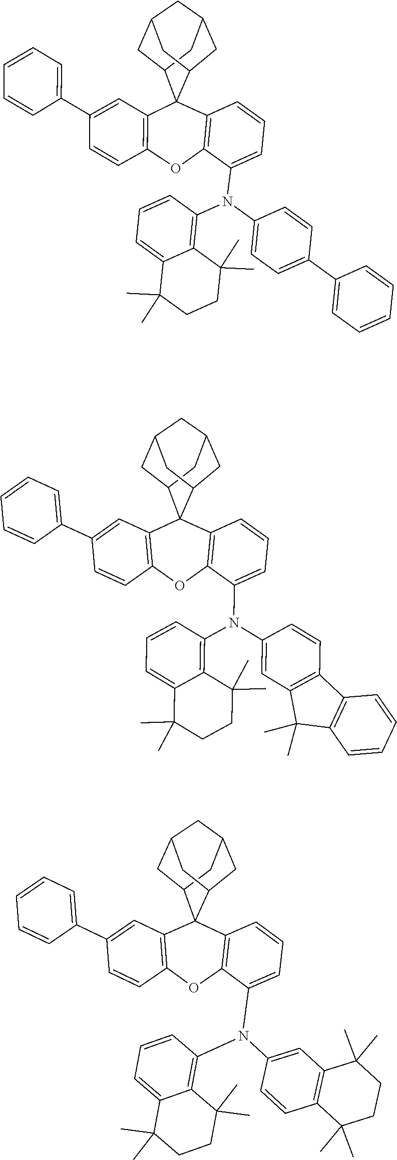 [SMILES: CC1(C)CCC(C)(C)c2c(N(c3ccc(-c4ccccc4)cc3)c3cccc4c3Oc3ccc(-c5ccccc5)cc3C43C4CC5CC(C4)CC3C5)cccc21 ; CC1(C)CCC(C)(C)c2c(N(c3ccc4c(c3)C(C)(C)c3ccccc3-4)c3cccc4c3Oc3ccc(-c5ccccc5)cc3C43C4CC5CC(C4)CC3C5)cccc21 ; CC1(C)CCC(C)(C)c2cc(N(c3cccc4c3Oc3ccc(-c5ccccc5)cc3C43C4CC5CC(C4)CC3C5)c3cccc4c3C(C)(C)CCC4(C)C)ccc21]

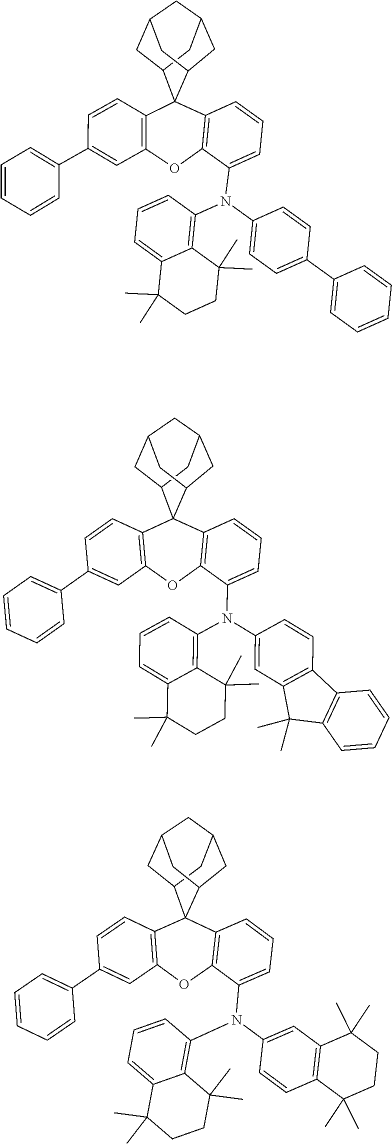 [SMILES: CC1(C)CCC(C)(C)c2c(N(c3ccc(-c4ccccc4)cc3)c3cccc4c3Oc3cc(-c5ccccc5)ccc3C43C4CC5CC(C4)CC3C5)cccc21 ; CC1(C)CCC(C)(C)c2c(N(c3ccc4c(c3)C(C)(C)c3ccccc3-4)c3cccc4c3Oc3cc(-c5ccccc5)ccc3C43C4CC5CC(C4)CC3C5)cccc21 ; CC1(C)CCC(C)(C)c2cc(N(c3cccc4c3Oc3cc(-c5ccccc5)ccc3C43C4CC5CC(C4)CC3C5)c3cccc4c3C(C)(C)CCC4(C)C)ccc21]

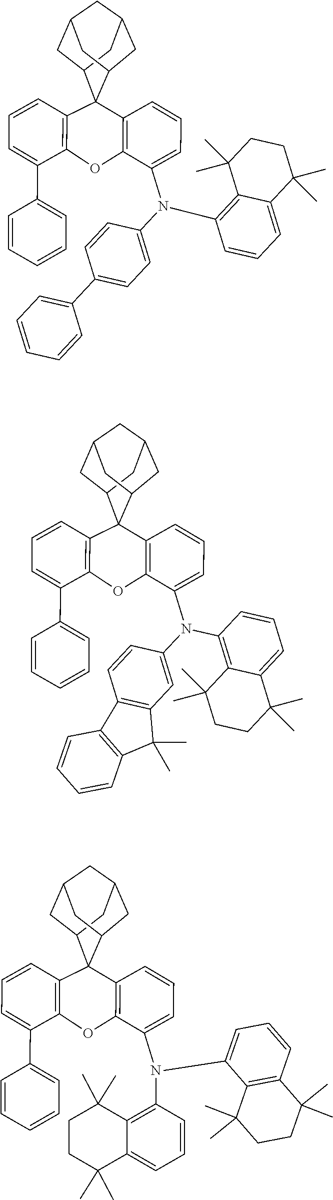 [SMILES: CC1(C)CCC(C)(C)c2c(N(c3ccc(-c4ccccc4)cc3)c3cccc4c3Oc3c(-c5ccccc5)cccc3C43C4CC5CC(C4)CC3C5)cccc21 ; CC1(C)CCC(C)(C)c2c(N(c3ccc4c(c3)C(C)(C)c3ccccc3-4)c3cccc4c3Oc3c(-c5ccccc5)cccc3C43C4CC5CC(C4)CC3C5)cccc21 ; CC1(C)CCC(C)(C)c2c(N(c3cccc4c3Oc3c(-c5ccccc5)cccc3C43C4CC5CC(C4)CC3C5)c3cccc4c3C(C)(C)CCC4(C)C)cccc21]

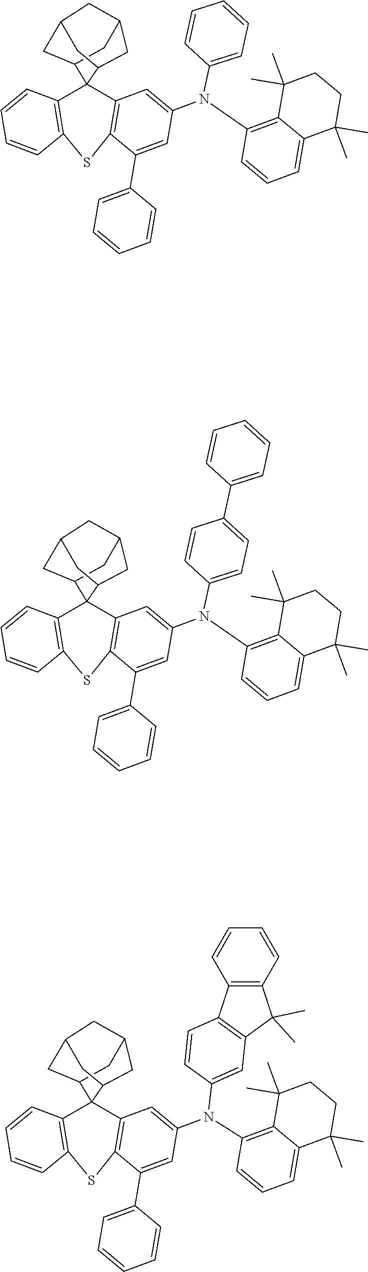 [SMILES: CC1(C)CCC(C)(C)c2c(N(c3ccccc3)c3cc(-c4ccccc4)c4c(c3)C3(c5ccccc5S4)C4CC5CC(C4)CC3C5)cccc21 ; CC1(C)CCC(C)(C)c2c(N(c3ccc(-c4ccccc4)cc3)c3cc(-c4ccccc4)c4c(c3)C3(c5ccccc5S4)C4CC5CC(C4)CC3C5)cccc21 ; CC1(C)CCC(C)(C)c2c(N(c3ccc4c(c3)C(C)(C)c3ccccc3-4)c3cc(-c4ccccc4)c4c(c3)C3(c5ccccc5S4)C4CC5CC(C4)CC3C5)cccc21]

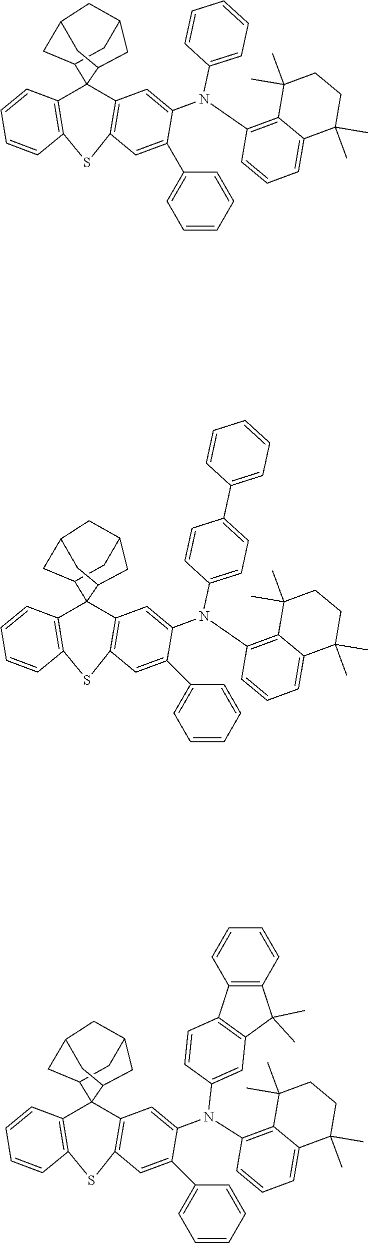 [SMILES: CC1(C)CCC(C)(C)c2c(N(c3ccccc3)c3cc4c(cc3-c3ccccc3)Sc3ccccc3C43C4CC5CC(C4)CC3C5)cccc21 ; CC1(C)CCC(C)(C)c2c(N(c3ccc(-c4ccccc4)cc3)c3cc4c(cc3-c3ccccc3)Sc3ccccc3C43C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)Sc3ccccc3C43C4CC5CC(C4)CC3C5)cccc21]

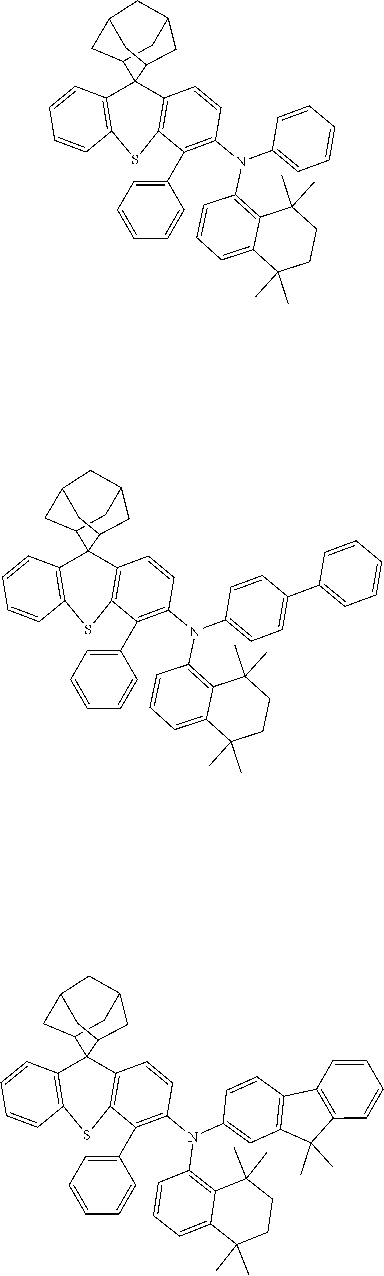 [SMILES: CC1(C)CCC(C)(C)c2c(N(c3ccccc3)c3ccc4c(c3-c3ccccc3)Sc3ccccc3C43C4CC5CC(C4)CC3C5)cccc21 ; CC1(C)CCC(C)(C)c2c(N(c3ccc(-c4ccccc4)cc3)c3ccc4c(c3-c3ccccc3)Sc3ccccc3C43C4CC5CC(C4)CC3C5)cccc21 ; CC1(C)CCC(C)(C)c2c(N(c3ccc4c(c3)C(C)(C)c3ccccc3-4)c3ccc4c(c3-c3ccccc3)Sc3ccccc3C43C4CC5CC(C4)CC3C5)cccc21]

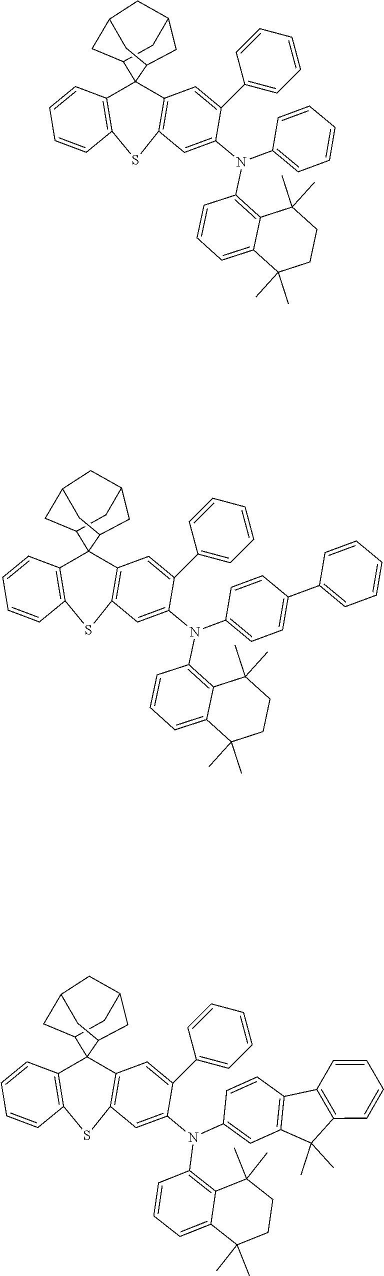 [SMILES: CC1(C)CCC(C)(C)c2c(N(c3ccccc3)c3cc4c(cc3-c3ccccc3)C3(c5ccccc5S4)C4CC5CC(C4)CC3C5)cccc21 ; CC1(C)CCC(C)(C)c2c(N(c3ccc(-c4ccccc4)cc3)c3cc4c(cc3-c3ccccc3)C3(c5ccccc5S4)C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)C3(c5ccccc5S4)C4CC5CC(C4)CC3C5)cccc21]

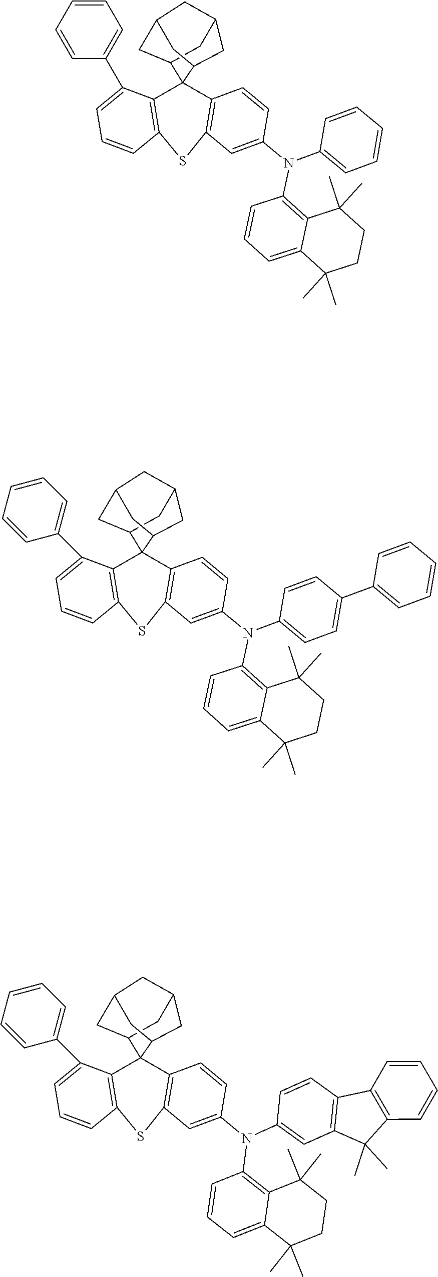 [SMILES: CC1(C)CCC(C)(C)c2c(N(c3ccccc3)c3ccc4c(c3)Sc3cccc(-c5ccccc5)c3C43C4CC5CC(C4)CC3C5)cccc21 ; CC1(C)CCC(C)(C)c2c(N(c3ccc(-c4ccccc4)cc3)c3ccc4c(c3)Sc3cccc(-c5ccccc5)c3C43C4CC5CC(C4)CC3C5)cccc21 ; CC1(C)CCC(C)(C)c2c(N(c3ccc4c(c3)Sc3cccc(-c5ccccc5)c3C43C4CC5CC(C4)CC3C5)c3ccc4c(c3)C(C)(C)c3ccccc3-4)cccc21]

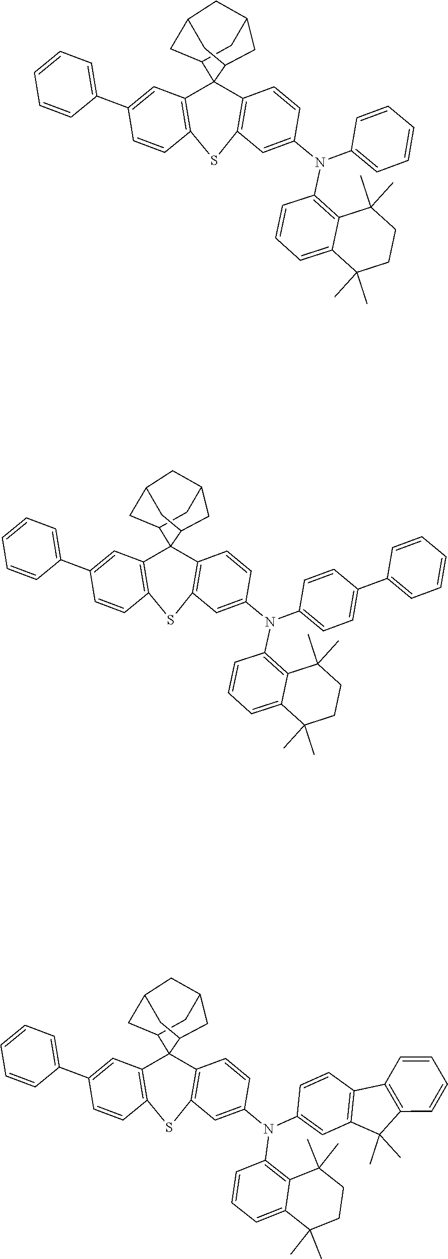 [SMILES: CC1(C)CCC(C)(C)c2c(N(c3ccccc3)c3ccc4c(c3)Sc3ccc(-c5ccccc5)cc3C43C4CC5CC(C4)CC3C5)cccc21 ; CC1(C)CCC(C)(C)c2c(N(c3ccc(-c4ccccc4)cc3)c3ccc4c(c3)Sc3ccc(-c5ccccc5)cc3C43C4CC5CC(C4)CC3C5)cccc21 ; CC1(C)CCC(C)(C)c2c(N(c3ccc4c(c3)Sc3ccc(-c5ccccc5)cc3C43C4CC5CC(C4)CC3C5)c3ccc4c(c3)C(C)(C)c3ccccc3-4)cccc21]

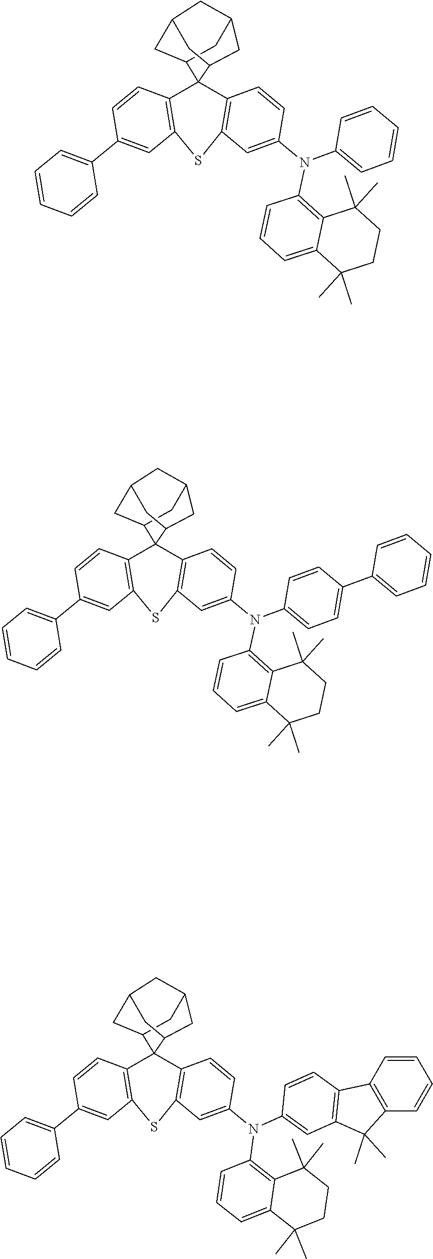 [SMILES: CC1(C)CCC(C)(C)c2c(N(c3ccccc3)c3ccc4c(c3)Sc3cc(-c5ccccc5)ccc3C43C4CC5CC(C4)CC3C5)cccc21 ; CC1(C)CCC(C)(C)c2c(N(c3ccc(-c4ccccc4)cc3)c3ccc4c(c3)Sc3cc(-c5ccccc5)ccc3C43C4CC5CC(C4)CC3C5)cccc21 ; CC1(C)CCC(C)(C)c2c(N(c3ccc4c(c3)Sc3cc(-c5ccccc5)ccc3C43C4CC5CC(C4)CC3C5)c3ccc4c(c3)C(C)(C)c3ccccc3-4)cccc21]

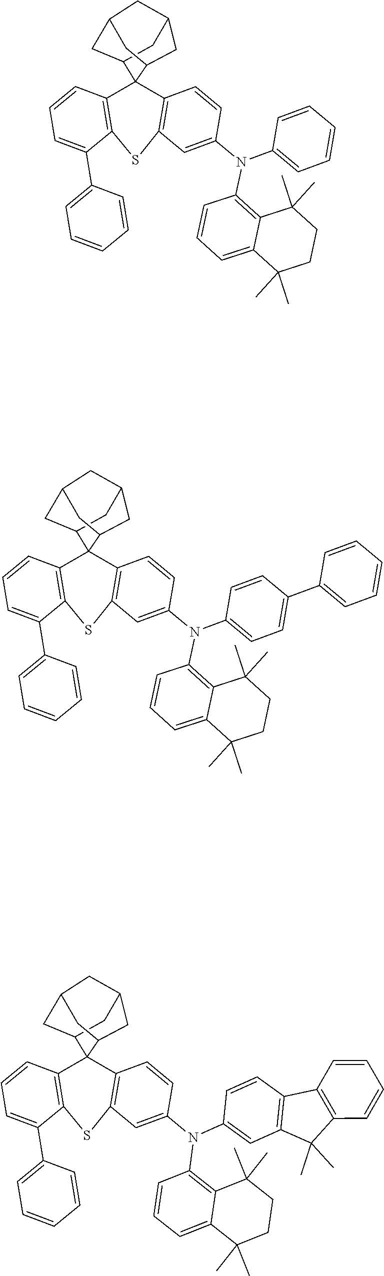 [SMILES: CC1(C)CCC(C)(C)c2c(N(c3ccccc3)c3ccc4c(c3)Sc3c(-c5ccccc5)cccc3C43C4CC5CC(C4)CC3C5)cccc21 ; CC1(C)CCC(C)(C)c2c(N(c3ccc(-c4ccccc4)cc3)c3ccc4c(c3)Sc3c(-c5ccccc5)cccc3C43C4CC5CC(C4)CC3C5)cccc21 ; CC1(C)CCC(C)(C)c2c(N(c3ccc4c(c3)Sc3c(-c5ccccc5)cccc3C43C4CC5CC(C4)CC3C5)c3ccc4c(c3)C(C)(C)c3ccccc3-4)cccc21]

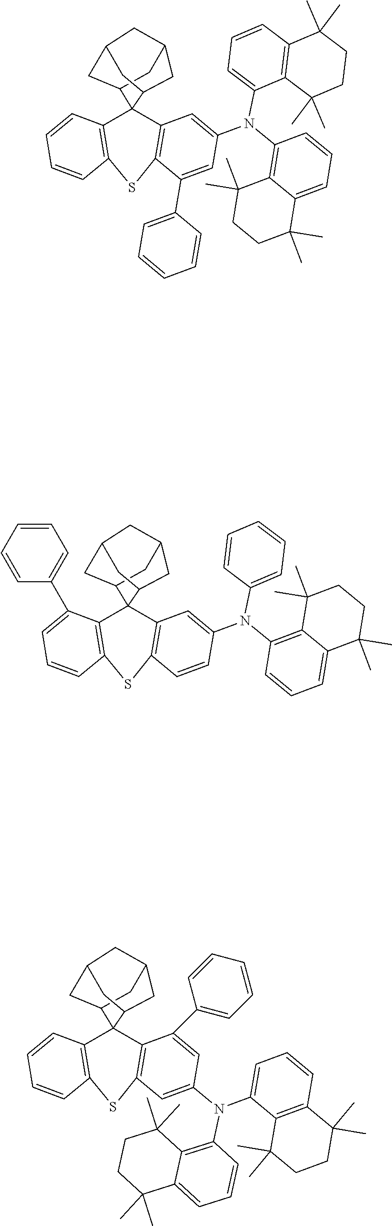 [SMILES: CC1(C)CCC(C)(C)c2c(N(c3cc(-c4ccccc4)c4c(c3)C3(c5ccccc5S4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5c(cccc5-c5ccccc5)S4)C4CC5CC(C4)CC3C5)cccc21 ; CC1(C)CCC(C)(C)c2c(N(c3cc4c(c(-c5ccccc5)c3)C3(c5ccccc5S4)C4CC5CC(C4)CC3C5)c3cccc4c3C(C)(C)CCC4(C)C)cccc21]

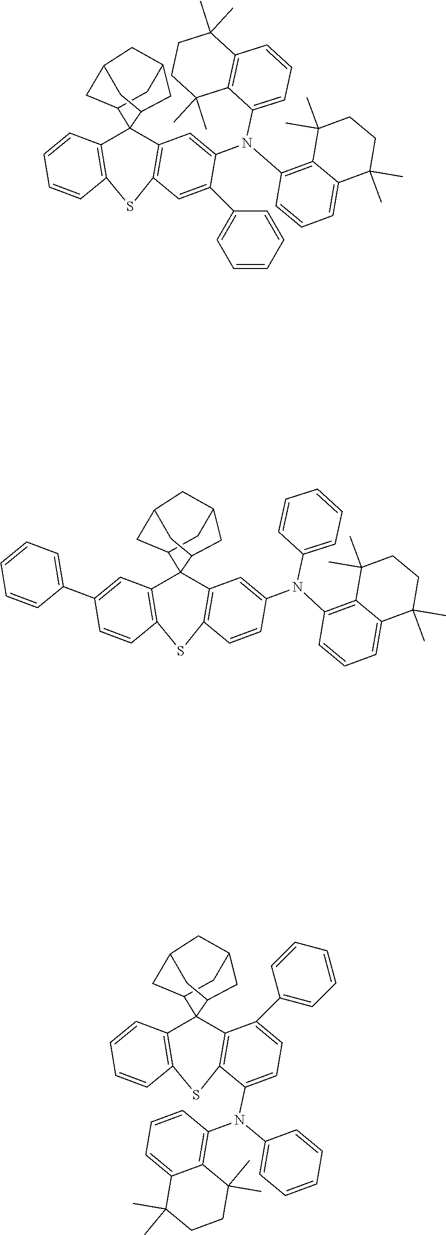 [SMILES: CC1(C)CCC(C)(C)c2c(N(c3cc4c(cc3-c3ccccc3)Sc3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cc(-c6ccccc6)ccc5S4)C4CC5CC(C4)CC3C5)cccc21 ; CC1(C)CCC(C)(C)c2c(N(c3ccccc3)c3ccc(-c4ccccc4)c4c3Sc3ccccc3C43C4CC5CC(C4)CC3C5)cccc21]

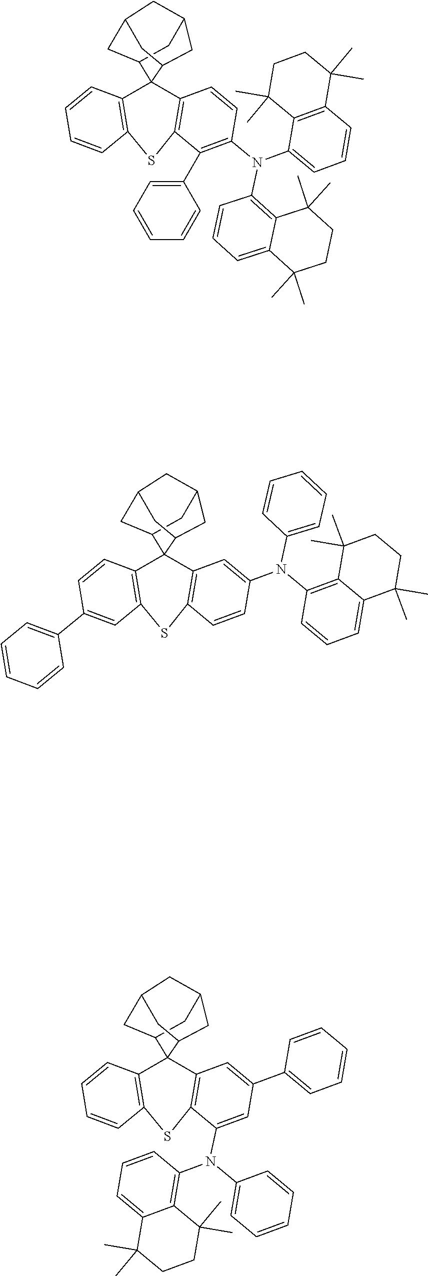 [SMILES: CC1(C)CCC(C)(C)c2c(N(c3ccc4c(c3-c3ccccc3)Sc3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5ccc(-c6ccccc6)cc5S4)C4CC5CC(C4)CC3C5)cccc21 ; CC1(C)CCC(C)(C)c2c(N(c3ccccc3)c3cc(-c4ccccc4)cc4c3Sc3ccccc3C43C4CC5CC(C4)CC3C5)cccc21]

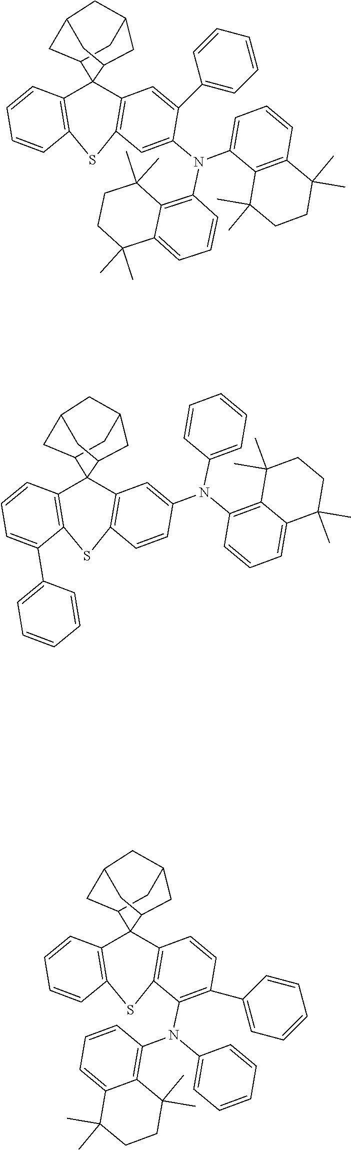 [SMILES: CC1(C)CCC(C)(C)c2c(N(c3cc4c(cc3-c3ccccc3)C3(c5ccccc5S4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cccc(-c6ccccc6)c5S4)C4CC5CC(C4)CC3C5)cccc21 ; CC1(C)CCC(C)(C)c2c(N(c3ccccc3)c3c(-c4ccccc4)ccc4c3Sc3ccccc3C43C4CC5CC(C4)CC3C5)cccc21]

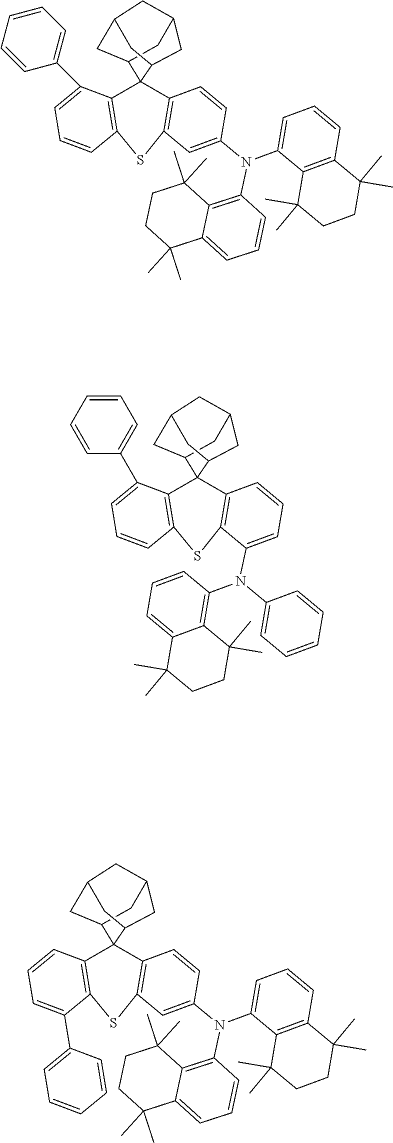 [SMILES: CC1(C)CCC(C)(C)c2c(N(c3ccc4c(c3)Sc3cccc(-c5ccccc5)c3C43C4CC5CC(C4)CC3C5)c3cccc4c3C(C)(C)CCC4(C)C)cccc21 ; CC1(C)CCC(C)(C)c2c(N(c3ccccc3)c3cccc4c3Sc3cccc(-c5ccccc5)c3C43C4CC5CC(C4)CC3C5)cccc21 ; CC1(C)CCC(C)(C)c2c(N(c3ccc4c(c3)Sc3c(-c5ccccc5)cccc3C43C4CC5CC(C4)CC3C5)c3cccc4c3C(C)(C)CCC4(C)C)cccc21]

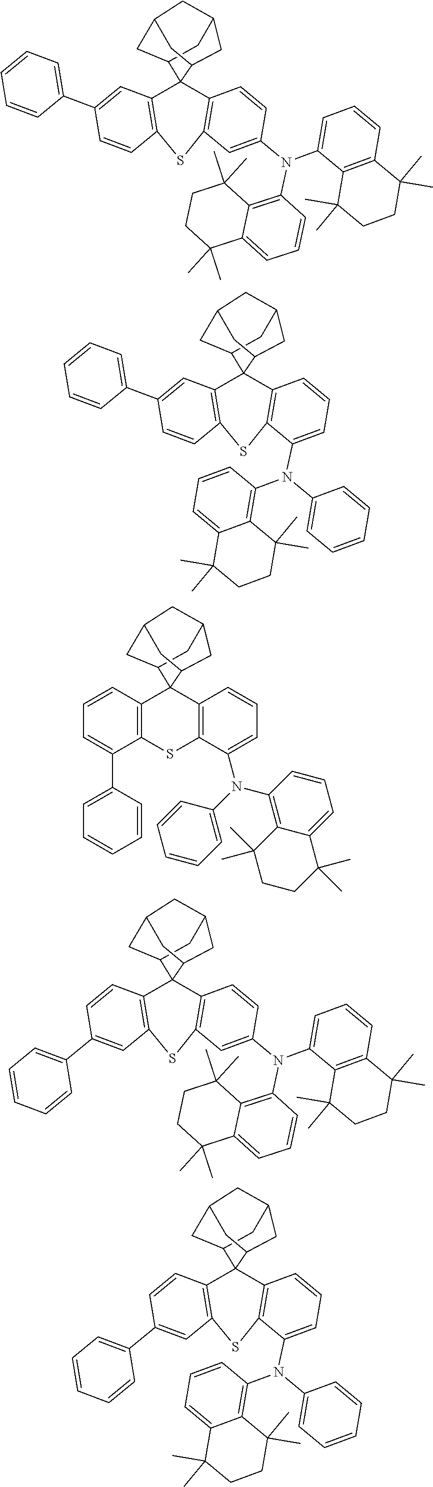

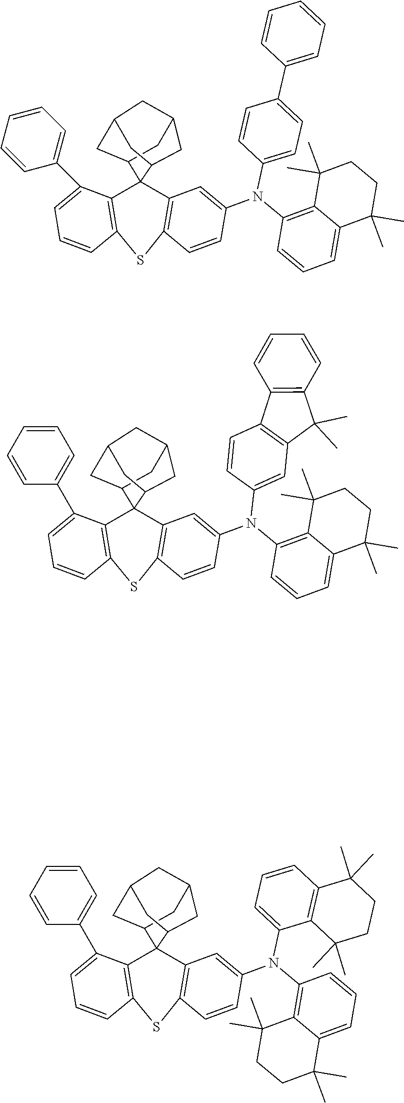 [SMILES: CC1(C)CCC(C)(C)c2c(N(c3ccc(-c4ccccc4)cc3)c3ccc4c(c3)C3(c5c(cccc5-c5ccccc5)S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5c(cccc5-c5ccccc5)S4)C4CC5CC(C4)CC3C5)cccc21 ; CC1(C)CCC(C)(C)c2c(N(c3ccc4c(c3)C3(c5c(cccc5-c5ccccc5)S4)C4CC5CC(C4)CC3C5)c3cccc4c3C(C)(C)CCC4(C)C)cccc21]

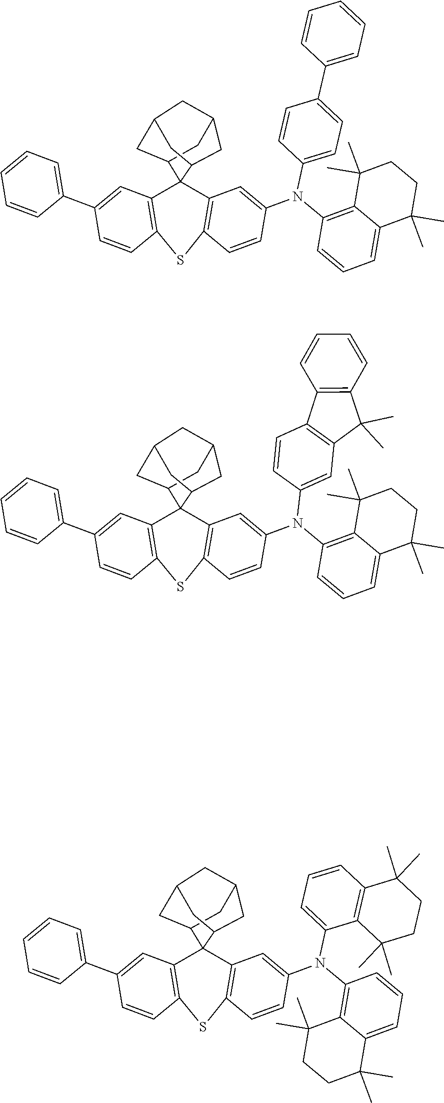 [SMILES: CC1(C)CCC(C)(C)c2c(N(c3ccc(-c4ccccc4)cc3)c3ccc4c(c3)C3(c5cc(-c6ccccc6)ccc5S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cc(-c6ccccc6)ccc5S4)C4CC5CC(C4)CC3C5)cccc21 ; CC1(C)CCC(C)(C)c2c(N(c3ccc4c(c3)C3(c5cc(-c6ccccc6)ccc5S4)C4CC5CC(C4)CC3C5)c3cccc4c3C(C)(C)CCC4(C)C)cccc21]

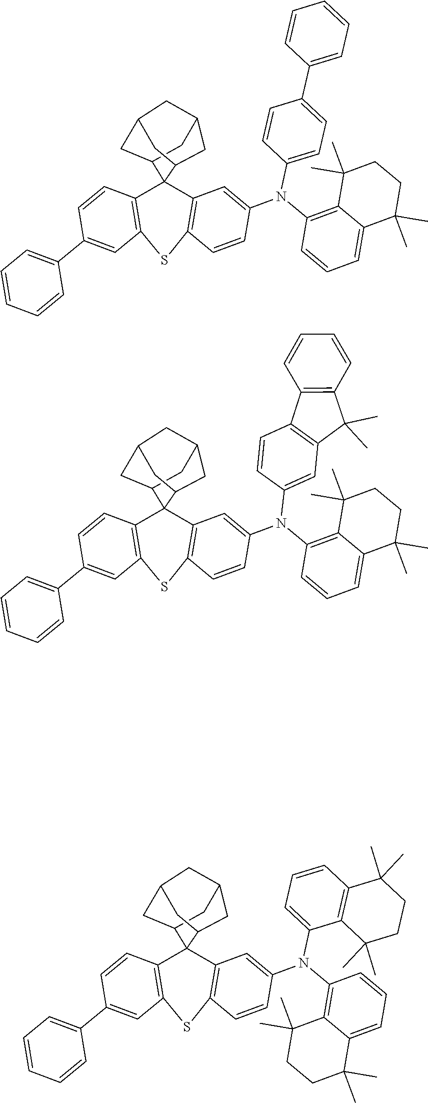 [SMILES: CC1(C)CCC(C)(C)c2c(N(c3ccc(-c4ccccc4)cc3)c3ccc4c(c3)C3(c5ccc(-c6ccccc6)cc5S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5ccc(-c6ccccc6)cc5S4)C4CC5CC(C4)CC3C5)cccc21 ; CC1(C)CCC(C)(C)c2c(N(c3ccc4c(c3)C3(c5ccc(-c6ccccc6)cc5S4)C4CC5CC(C4)CC3C5)c3cccc4c3C(C)(C)CCC4(C)C)cccc21]

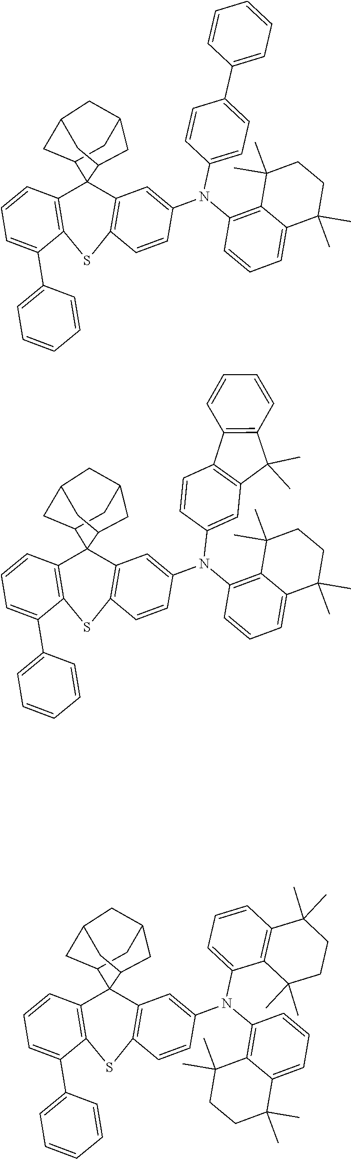 [SMILES: CC1(C)CCC(C)(C)c2c(N(c3ccc(-c4ccccc4)cc3)c3ccc4c(c3)C3(c5cccc(-c6ccccc6)c5S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cccc(-c6ccccc6)c5S4)C4CC5CC(C4)CC3C5)cccc21 ; CC1(C)CCC(C)(C)c2c(N(c3ccc4c(c3)C3(c5cccc(-c6ccccc6)c5S4)C4CC5CC(C4)CC3C5)c3cccc4c3C(C)(C)CCC4(C)C)cccc21]

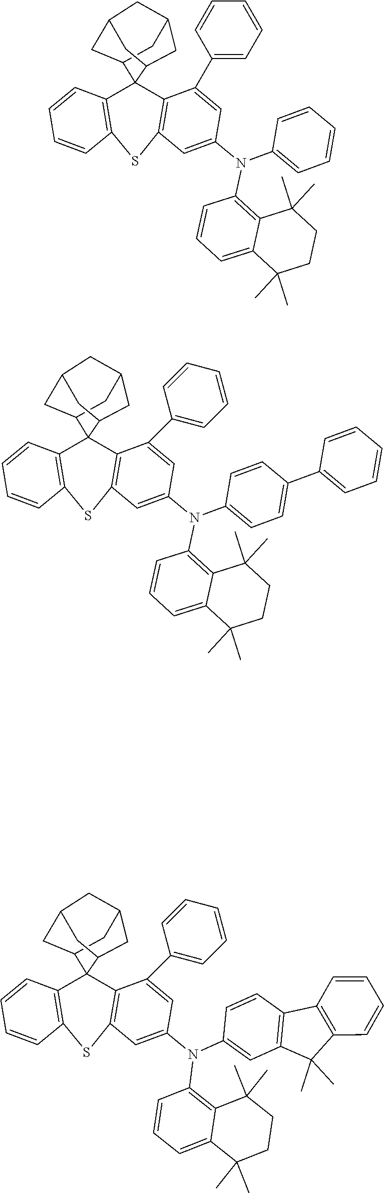 [SMILES: CC1(C)CCC(C)(C)c2c(N(c3ccccc3)c3cc4c(c(-c5ccccc5)c3)C3(c5ccccc5S4)C4CC5CC(C4)CC3C5)cccc21 ; CC1(C)CCC(C)(C)c2c(N(c3ccc(-c4ccccc4)cc3)c3cc4c(c(-c5ccccc5)c3)C3(c5ccccc5S4)C4CC5CC(C4)CC3C5)cccc21 ; CC1(C)CCC(C)(C)c2c(N(c3cc4c(c(-c5ccccc5)c3)C3(c5ccccc5S4)C4CC5CC(C4)CC3C5)c3ccc4c(c3)C(C)(C)c3ccccc3-4)cccc21]

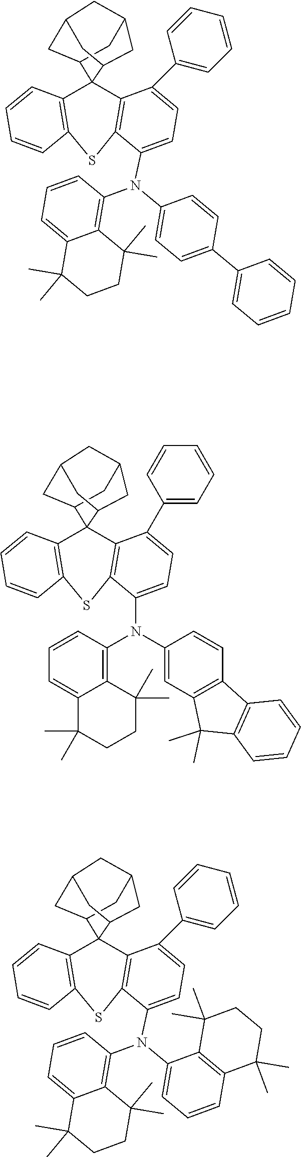 [SMILES: CC1(C)CCC(C)(C)c2c(N(c3ccc(-c4ccccc4)cc3)c3ccc(-c4ccccc4)c4c3Sc3ccccc3C43C4CC5CC(C4)CC3C5)cccc21 ; CC1(C)CCC(C)(C)c2c(N(c3ccc4c(c3)C(C)(C)c3ccccc3-4)c3ccc(-c4ccccc4)c4c3Sc3ccccc3C43C4CC5CC(C4)CC3C5)cccc21 ; CC1(C)CCC(C)(C)c2c(N(c3ccc(-c4ccccc4)c4c3Sc3ccccc3C43C4CC5CC(C4)CC3C5)c3cccc4c3C(C)(C)CCC4(C)C)cccc21]

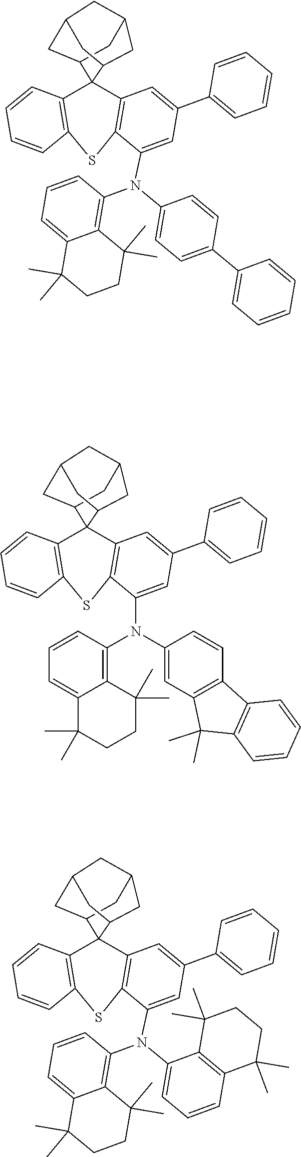 [SMILES: CC1(C)CCC(C)(C)c2c(N(c3ccc(-c4ccccc4)cc3)c3cc(-c4ccccc4)cc4c3Sc3ccccc3C43C4CC5CC(C4)CC3C5)cccc21 ; CC1(C)CCC(C)(C)c2c(N(c3ccc4c(c3)C(C)(C)c3ccccc3-4)c3cc(-c4ccccc4)cc4c3Sc3ccccc3C43C4CC5CC(C4)CC3C5)cccc21 ; CC1(C)CCC(C)(C)c2c(N(c3cc(-c4ccccc4)cc4c3Sc3ccccc3C43C4CC5CC(C4)CC3C5)c3cccc4c3C(C)(C)CCC4(C)C)cccc21]

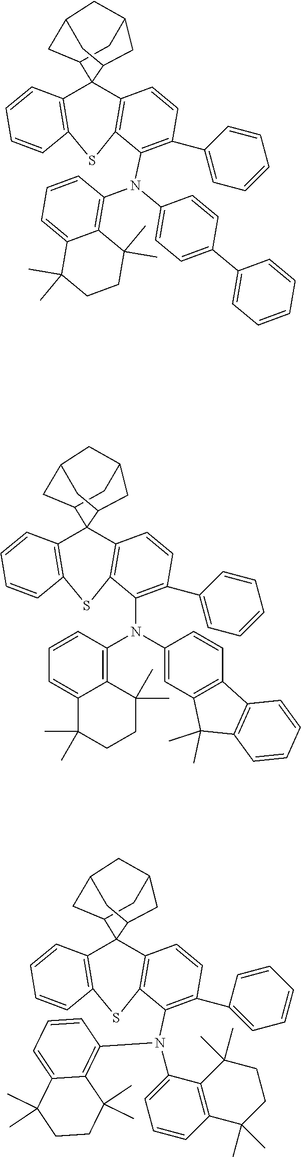 [SMILES: CC1(C)CCC(C)(C)c2c(N(c3ccc(-c4ccccc4)cc3)c3c(-c4ccccc4)ccc4c3Sc3ccccc3C43C4CC5CC(C4)CC3C5)cccc21 ; CC1(C)CCC(C)(C)c2c(N(c3ccc4c(c3)C(C)(C)c3ccccc3-4)c3c(-c4ccccc4)ccc4c3Sc3ccccc3C43C4CC5CC(C4)CC3C5)cccc21 ; CC1(C)CCC(C)(C)c2c(N(c3cccc4c3C(C)(C)CCC4(C)C)c3c(-c4ccccc4)ccc4c3Sc3ccccc3C43C4CC5CC(C4)CC3C5)cccc21]

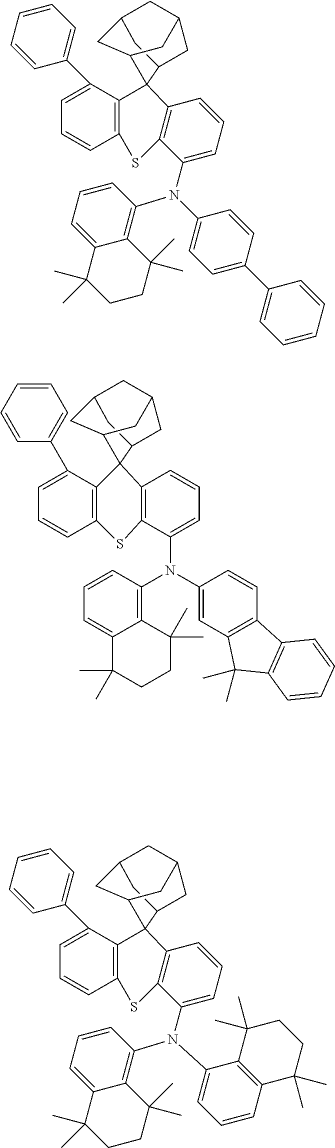 [SMILES: CC1(C)CCC(C)(C)c2c(N(c3ccc(-c4ccccc4)cc3)c3cccc4c3Sc3cccc(-c5ccccc5)c3C43C4CC5CC(C4)CC3C5)cccc21 ; CC1(C)CCC(C)(C)c2c(N(c3ccc4c(c3)C(C)(C)c3ccccc3-4)c3cccc4c3Sc3cccc(-c5ccccc5)c3C43C4CC5CC(C4)CC3C5)cccc21 ; CC1(C)CCC(C)(C)c2c(N(c3cccc4c3Sc3cccc(-c5ccccc5)c3C43C4CC5CC(C4)CC3C5)c3cccc4c3C(C)(C)CCC4(C)C)cccc21]

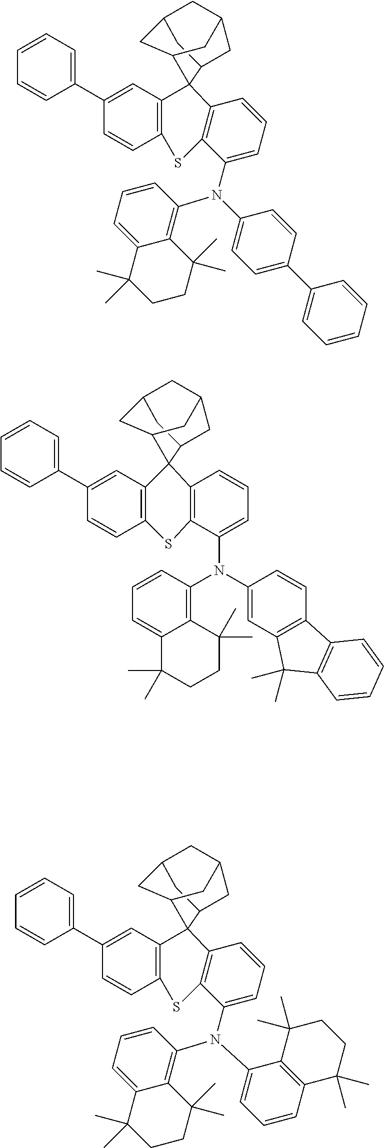 [SMILES: CC1(C)CCC(C)(C)c2c(N(c3ccc(-c4ccccc4)cc3)c3cccc4c3Sc3ccc(-c5ccccc5)cc3C43C4CC5CC(C4)CC3C5)cccc21 ; CC1(C)CCC(C)(C)c2c(N(c3ccc4c(c3)C(C)(C)c3ccccc3-4)c3cccc4c3Sc3ccc(-c5ccccc5)cc3C43C4CC5CC(C4)CC3C5)cccc21 ; CC1(C)CCC(C)(C)c2c(N(c3cccc4c3Sc3ccc(-c5ccccc5)cc3C43C4CC5CC(C4)CC3C5)c3cccc4c3C(C)(C)CCC4(C)C)cccc21]

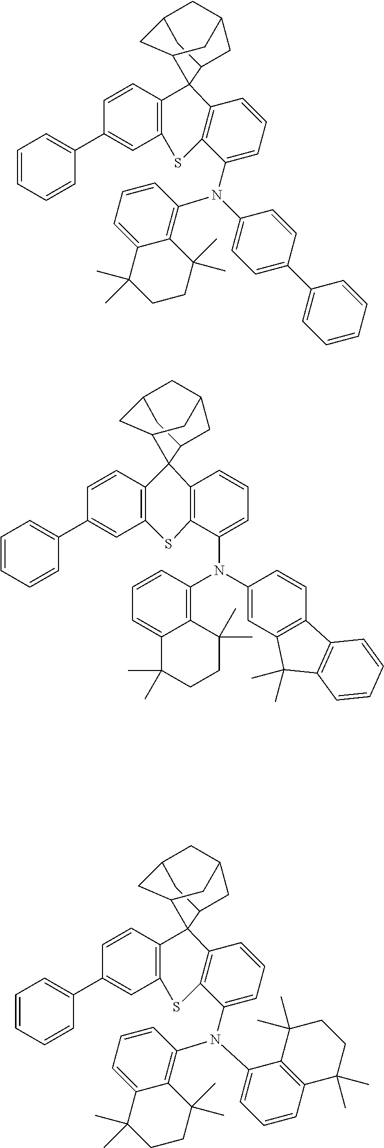 [SMILES: CC1(C)CCC(C)(C)c2c(N(c3ccc(-c4ccccc4)cc3)c3cccc4c3Sc3cc(-c5ccccc5)ccc3C43C4CC5CC(C4)CC3C5)cccc21 ; CC1(C)CCC(C)(C)c2c(N(c3ccc4c(c3)C(C)(C)c3ccccc3-4)c3cccc4c3Sc3cc(-c5ccccc5)ccc3C43C4CC5CC(C4)CC3C5)cccc21 ; CC1(C)CCC(C)(C)c2c(N(c3cccc4c3Sc3cc(-c5ccccc5)ccc3C43C4CC5CC(C4)CC3C5)c3cccc4c3C(C)(C)CCC4(C)C)cccc21]

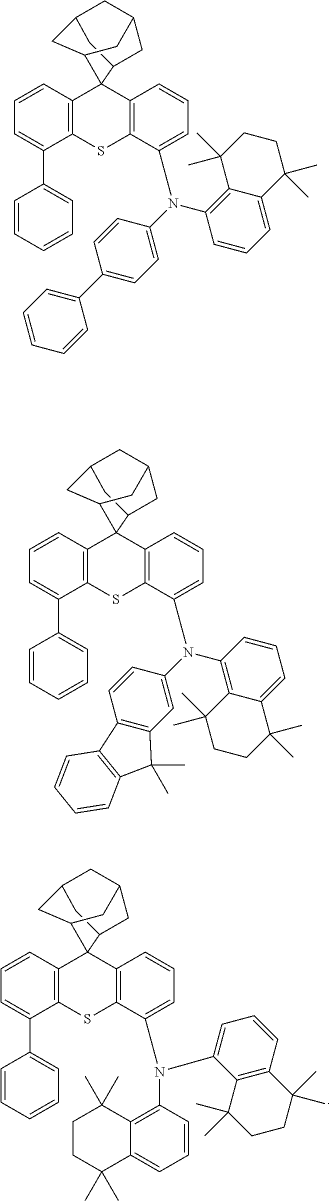 [SMILES: CC1(C)CCC(C)(C)c2c(N(c3ccc(-c4ccccc4)cc3)c3cccc4c3Sc3c(-c5ccccc5)cccc3C43C4CC5CC(C4)CC3C5)cccc21 ; CC1(C)CCC(C)(C)c2c(N(c3ccc4c(c3)C(C)(C)c3ccccc3-4)c3cccc4c3Sc3c(-c5ccccc5)cccc3C43C4CC5CC(C4)CC3C5)cccc21 ; CC1(C)CCC(C)(C)c2c(N(c3cccc4c3Sc3c(-c5ccccc5)cccc3C43C4CC5CC(C4)CC3C5)c3cccc4c3C(C)(C)CCC4(C)C)cccc21]

For example, the compound represented by the Chemical Formula 1 may be prepared by the preparation method of Reaction Scheme 1 below, and the other compounds can be prepared similarly:

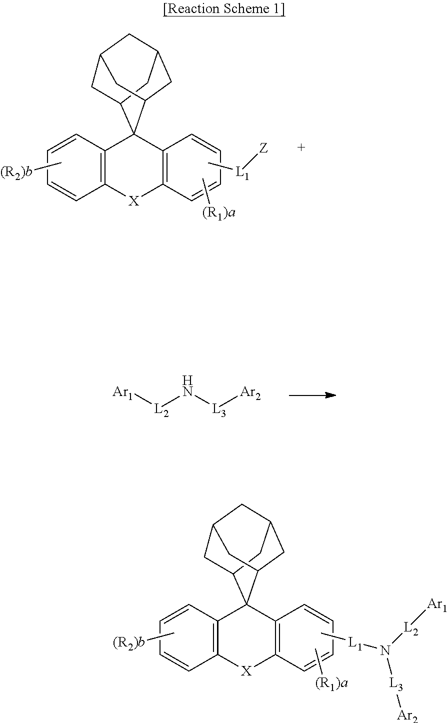

In the Reaction Scheme 1, definitions of X, R₁, R₂, a, b, L₁ to L₃, Ar₁ and Ar₂ are the same as defined above, and Z is halogen, preferably chloro, or bromo.

The reaction scheme 1 is an amine substitution reaction, and preferably performed in the presence of a palladium catalyst and a base, and the reactive group for the amine substitution reaction may be appropriately changed. The preparation method may be more specifically described in Preparation Examples described below.

In addition, there is provided an organic light emitting device including the compound represented by the Chemical Formula 1. As an example, there is provided an organic light emitting device including: a first electrode; a second electrode that is provided opposite to the first electrode; and one or more organic material layers that are provided between the first electrode and the second electrode, wherein at least one layer of the organic material layers includes the compound represented by the Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure including a hole injection layer, a hole transport layer, an electron blocking layer, a light emitting layer, an electron transport layer, an electron injection layer, and the like as the organic material layer. However, the structure of the organic light emitting device is not limited thereto, and it may include more or fewer organic material layers.

In addition, the organic material layer may include a hole transport layer, a hole injection layer, a hole transport and injection layer, or an electron blocking layer, and the hole transport layer, the hole injection layer, the hole transport and injection layer, or the electron blocking layer may include the compound represented by the Chemical Formula 1.

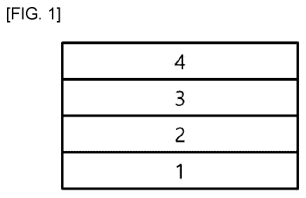

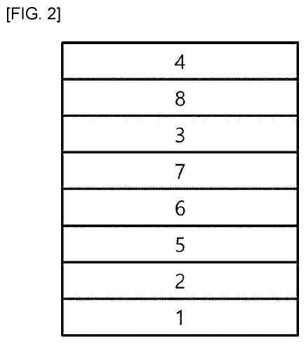

Further, the organic light emitting device according to the present disclosure may be a normal type organic light emitting device in which an anode, one or more organic material layers and a cathode are sequentially stacked on a substrate. Further, the organic light emitting device according to the present disclosure may be an inverted type organic light emitting device in which a cathode, one or more organic material layers and an anode are sequentially stacked on a substrate. For example, the structure of an organic light emitting device according to an embodiment of the present disclosure is illustrated in FIGS. 1 and 2.

FIG. 1 shows an example of an organic light emitting device including a substrate 1, an anode 2, a light emitting layer 3, and a cathode 4. In such a structure, the compound represented by the Chemical Formula 1 may be included in the light emitting layer.

FIG. 2 shows an example of an organic light emitting device including a substrate 1, an anode 2, a hole injection layer 5, a hole transport layer 6, an electron blocking layer 7, a light emitting layer 3, an electron injection and transport layer 8, and a cathode 4. In this structure, the compound represented by the Chemical Formula 1 may be included in the hole transport layer or the electron blocking layer.

The organic light emitting device according to the present disclosure may be manufactured using materials and methods known in the art, except that at least one layer of the organic material layers includes the compound represented by the Chemical Formula 1. Moreover, when the organic light emitting device includes a plurality of organic material layers, the organic material layers may be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the electron blocking layer, the light emitting layer and the electron transport layer thereon, and then depositing a material that can be used as the cathode thereon. In addition to such a method, the organic light emitting device may be manufactured by sequentially depositing a cathode material, an organic material layer and an anode material on a substrate.

Further, the compound represented by the Chemical Formula 1 may be formed into an organic material layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

For example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene] (PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

In addition, the hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto. Preferably, the compound represented by the Chemical Formula 1 may be used as the hole transport layer material.

Meanwhile, in the present disclosure, the “hole injection and transport layer” is a layer that performs the functions of both the hole injection layer and the hole transport layer. The materials of each layer can be used alone or in combination, but are limited thereto.

The electron blocking layer is a layer placed between a hole transport layer and a light emitting layer to prevent electrons injected from the cathode from being transferred to the hole transport layer without recombination in the light emitting layer, and is also called an electron suppressing layer. A material having the electron affinity lower than that of the electron transport layer is preferable for the electron blocking layer. Preferably, the compound represented by the Chemical Formula 1 may be used as the electron blocking layer material.

The light emitting material is suitably a material capable of emitting light in a visible ray region by receiving holes and electrons from the hole transport layer and the electron transport layer, respectively, to combine them, and having good quantum efficiency to fluorescence or phosphorescence. Specific examples thereof include 8-hydroxy-quinoline aluminum complex (Alq₃); a carbazole-based compound; a dimerized styryl compound; BAlq; a 10-hydroxybenzo quinoline-metal compound; a benzoxazole-, benzthiazole- and benzimidazole-based compound; a poly(p-phenylenevinylene) (PPV)-based polymer; a spiro compound; polyfluorene, rubrene, and the like, but are not limited thereto.

The light emitting layer may include a host material and a dopant material. The host material may be a fused aromatic ring derivative or a heterocycle-containing compound. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

The dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material used is suitably a material which can receive electrons well from a cathode and transfer the electrons to a light emitting layer and has large mobility for electrons. Specifically, examples thereof may include an Al complex of 8-hydroxyquinoline; a complex including Alq₃; an organic radical compound; a hydroxyflavone-metal complex, and the like, but are not limited thereto. The electron transport layer may be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron injection and transport layer” is a layer that performs the functions of both the electron injection layer and the electron transport layer. The materials of each layer can be used alone or in combination, but are limited thereto.

The organic light emitting device according to the present disclosure may be a bottom emission device, a top emission device, or a double-sided emission device, and particularly a bottom emission device requiring high luminous efficiency.

In addition, the compound represented by the Chemical Formula 1 may be included in an organic solar cell or an organic transistor in addition to an organic light emitting device.

The preparation of the compound represented by the Chemical Formula 1 and the organic light emitting device including the same will be described in detail in the following examples. However, these examples are presented for illustrative purposes only, and are not intended to limit the scope of the present disclosure.

### SYNTHESIS EXAMPLE

### Synthesis Example 1. Synthesis of Compound 1

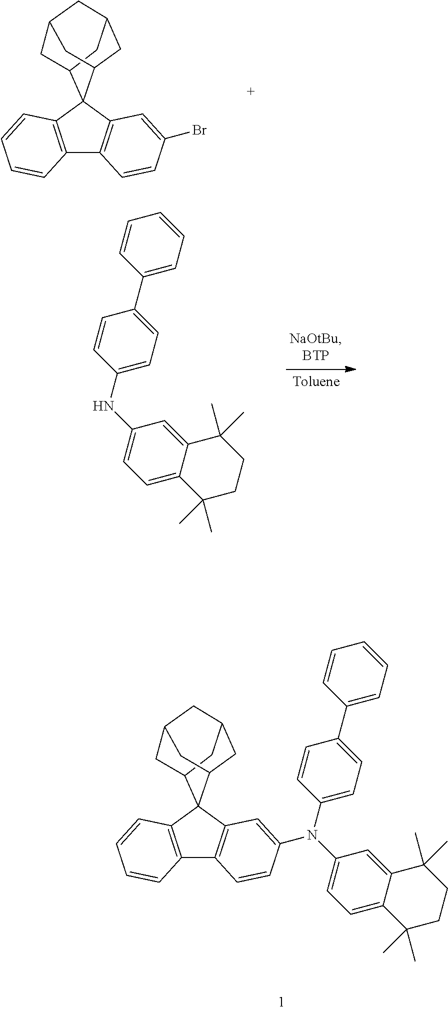

2′-Bromospiro[adamanthane-2,9′-fluorene] (20.0 g, 54.75 mmol) and N-([1,1′-biphenyl]-4-yl)-5,5,8,8-tetramethyl-5,6,7,8-tetrahydronaphthalen-2-amine (19.85 g, 55.84 mmol), and sodium tert-butoxide (7.37 g, 76.65 mmol) were added to toluene (300 ml), followed by heating and stirring for 10 minutes. Bis(tri-tert-butylphosphine) palladium (0.14 g, 0.27 mmol) dissolved in toluene (30 ml) was added to the mixture, followed by heating and stirring for 1 hour. After completion of the reaction and filtration, layer separation was performed with toluene and water. After removal of the solvent, the compound 1 (27.5 g, 78.49% yield) was obtained by recrystallization with ethyl acetate. (MS: [M+H]⁺=640)

### Synthesis Example 2. Synthesis of Compound 2

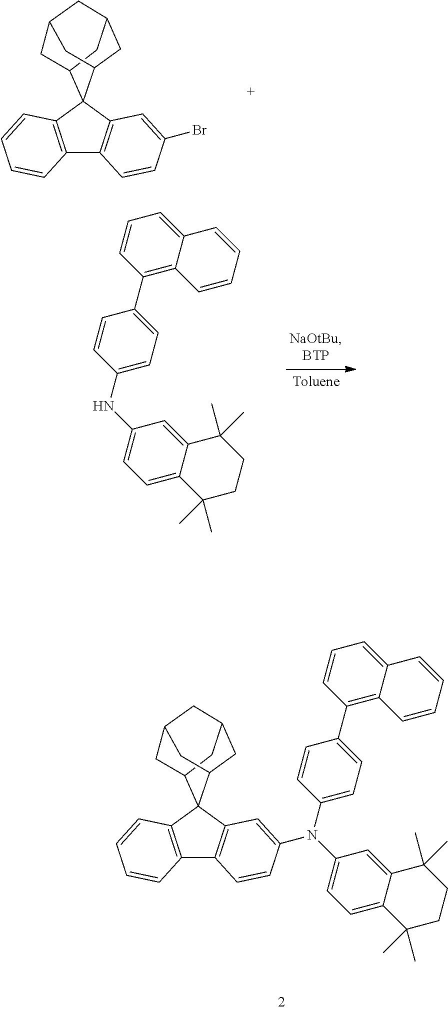

Compound 2 (29.5 g, 78.09% yield) was obtained in the same manner as in Synthesis Example 1 using 2′-bromospiro[adamanthane-2,9′-fluorene] (20.0 g, 54.75 mmol) and 5,5,8,8-tetramethyl-N-(4-(naphthalen-1-yl)phenyl)-5,6,7,8-tetrahydronaphthalen-2-amine (22.65 g, 55.84 mmol). (MS: [M+H]⁺=690)

### Synthesis Example 3. Synthesis of Compound 3

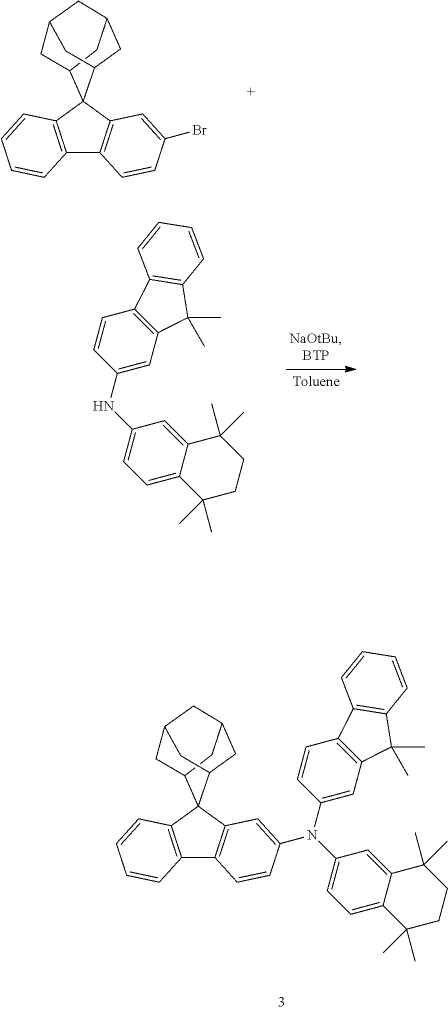 [SMILES: Brc1ccc2c(c1)C1(c3ccccc3-2)C2CC3CC(C2)CC1C3 ; CC1(C)CCC(C)(C)c2cc(Nc3ccc4c(c3)C(C)(C)c3ccccc3-4)ccc21 ; CC1(C)CCC(C)(C)c2cc(N(c3ccc4c(c3)C(C)(C)c3ccccc3-4)c3ccc4c(c3)C3(c5ccccc5-4)C4CC5CC(C4)CC3C5)ccc21]

Compound 3 (28.5 g, 76.55% yield) was obtained in the same manner as in Synthesis Example 1 using 2′-bromospiro[adamanthane-2,9′-fluorene] (20.0 g, 54.75 mmol) and 9,9-dimethyl-N-(5,5,8,8-tetramethyl-5,6,7,8-tetrahydronaphthalen-2-yl)-9H-fluoren-2-amine (22.09 g, 55.84 mmol). (MS: [M+H]⁺=680)

### Synthesis Example 4. Synthesis of Compound 4

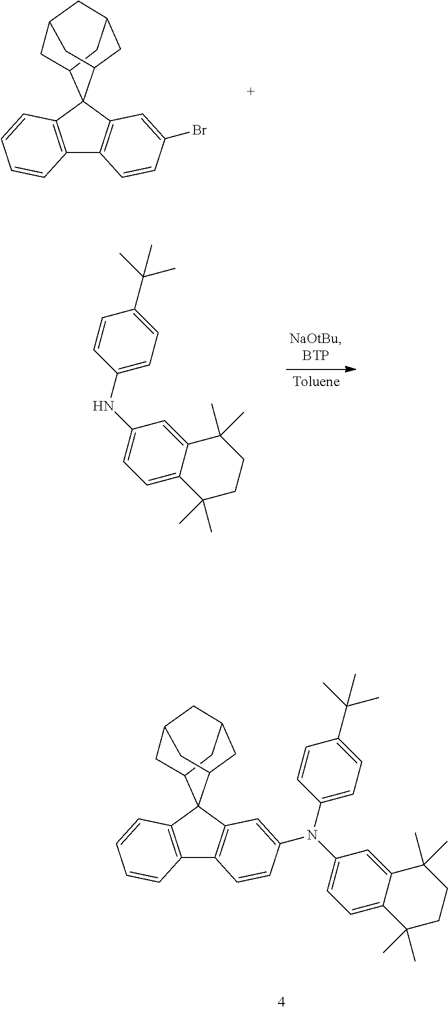 [SMILES: Brc1ccc2c(c1)C1(c3ccccc3-2)C2CC3CC(C2)CC1C3 ; CC(C)(C)c1ccc(Nc2ccc3c(c2)C(C)(C)CCC3(C)C)cc1 ; CC(C)(C)c1ccc(N(c2ccc3c(c2)C(C)(C)CCC3(C)C)c2ccc3c(c2)C2(c4ccccc4-3)C3CC4CC(C3)CC2C4)cc1]

Compound 4 (26.5 g, 78.08% yield) was obtained in the same manner as in Synthesis Example 1 using 2′-bromospiro[adamanthane-2,9′-fluorene] (20.0 g, 54.75 mmol) and N-(4-(tert-butyl)phenyl)-5,5,8,8-tetramethyl-5,6,7,8-tetrahydronaphthalen-2-amine (19.85 g, 55.84 mmol). (MS: [M+H]⁺=620)

### Synthesis Example 5. Synthesis of Compound 5

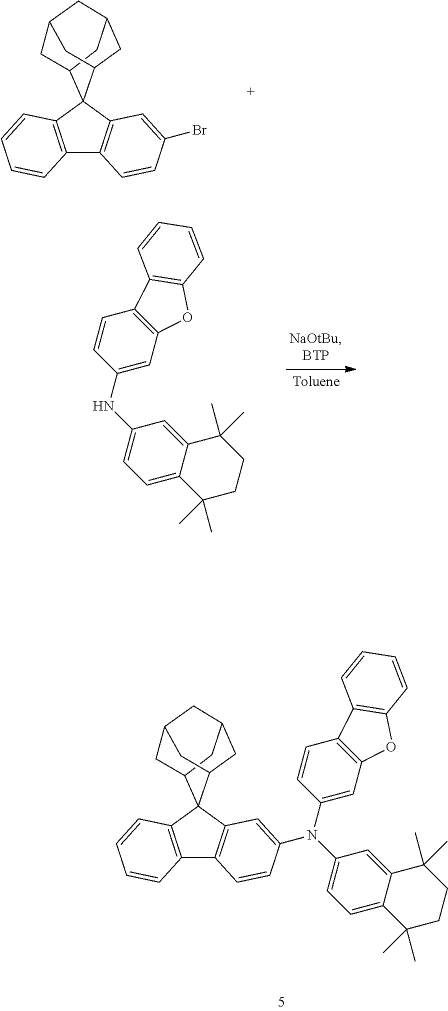 [SMILES: Brc1ccc2c(c1)C1(c3ccccc3-2)C2CC3CC(C2)CC1C3 ; CC1(C)CCC(C)(C)c2cc(Nc3ccc4c(c3)oc3ccccc34)ccc21 ; CC1(C)CCC(C)(C)c2cc(N(c3ccc4c(c3)C3(c5ccccc5-4)C4CC5CC(C4)CC3C5)c3ccc4c(c3)oc3ccccc34)ccc21]

Compound 5 (28.0 g, 78.21% yield) was obtained in the same manner as in Synthesis Example 1 using 2′-bromospiro[adamanthane-2,9′-fluorene] (20.0 g, 54.75 mmol) and N-(5,5,8,8-tetramethyl-5,6,7,8-tetrahydronaphthalen-2-yl)dibenzo[b,d]furan-3-amine (20.63 g, 55.84 mmol). (MS: [M+H]⁺=654)

### Synthesis Example 6. Synthesis of Compound 6

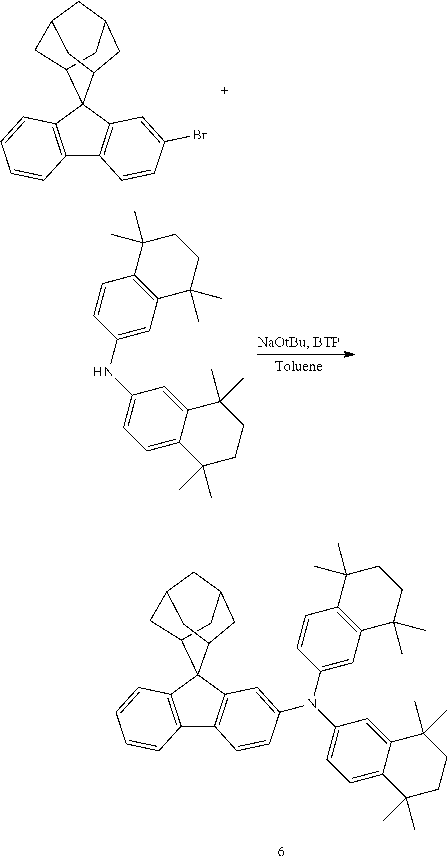

Compound 6 (29.0 g, 78.58 yield) was obtained in the same manner as in Synthesis Example 1 using 2′-bromospiro[adamanthane-2,9′-fluorene] (20.0 g, 54.75 mmol) and bis(5,5,8,8-tetramethyl-5,6,7,8-tetrahydronaphthalen-2-yl)amine (21.76 g, 55.84 mmol). (MS: [M+H]⁺=674)

### Synthesis Example 7. Synthesis of Compound 7

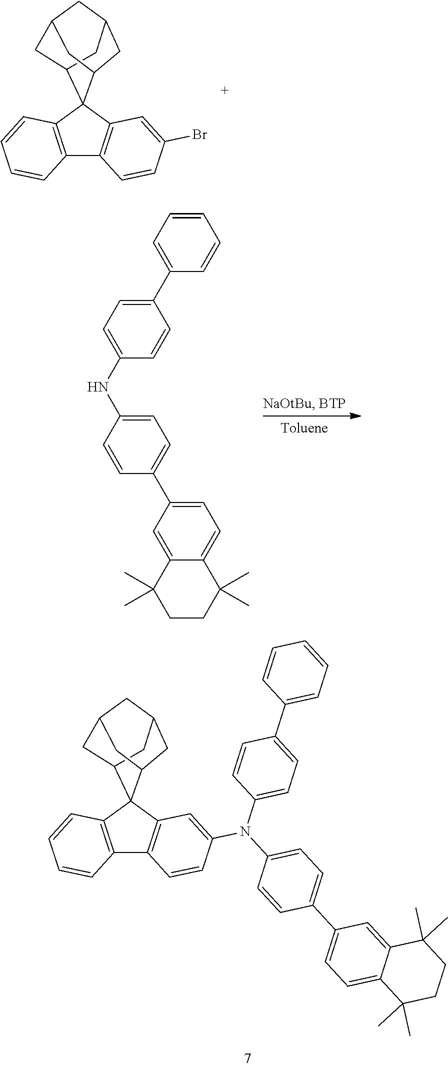

Compound 7 (30.5 g, 77.80% yield) was obtained in the same manner as in Synthesis Example 1 using 2′-bromospiro[adamanthane-2,9′-fluorene] (20.0 g, 54.75 mmol) and N-(5,5,8,8-tetramethyl-5,6,7,8-tetrahydronaphthalen-2-yl)phenyl)-[1,1′-biphenyl]-4-amine (24.10 g, 55.84 mmol). (MS: [M+H]⁺=716)

### Synthesis Example 8. Synthesis of Compound 8

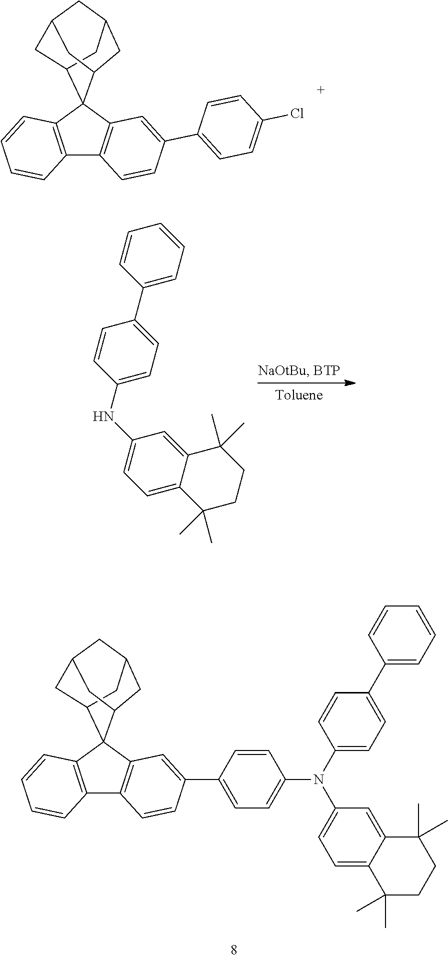 [SMILES: Clc1ccc(-c2ccc3c(c2)C2(c4ccccc4-3)C3CC4CC(C3)CC2C4)cc1 ; CC1(C)CCC(C)(C)c2cc(Nc3ccc(-c4ccccc4)cc3)ccc21 ; CC1(C)CCC(C)(C)c2cc(N(c3ccc(-c4ccccc4)cc3)c3ccc(-c4ccc5c(c4)C4(c6ccccc6-5)C5CC6CC(C5)CC4C6)cc3)ccc21]

Compound 8 (28.5 g, 79.01% yield) was obtained in the same manner as in Synthesis Example 1 using 2′-(4-chlorophenyl)spiro[adamanthane-2,9′-fluorene] (20.0 g, 50.38 mmol) and N-([1,1′-biphenyl]-4-yl)-5,5,8,8-tetramethyl-5,6,7,8-tetrahydronaphthalen-2-amine (18.27 g, 51.39 mmol). (MS: [M+H]⁺=716)

### Synthesis Example 9. Synthesis of Compound 9

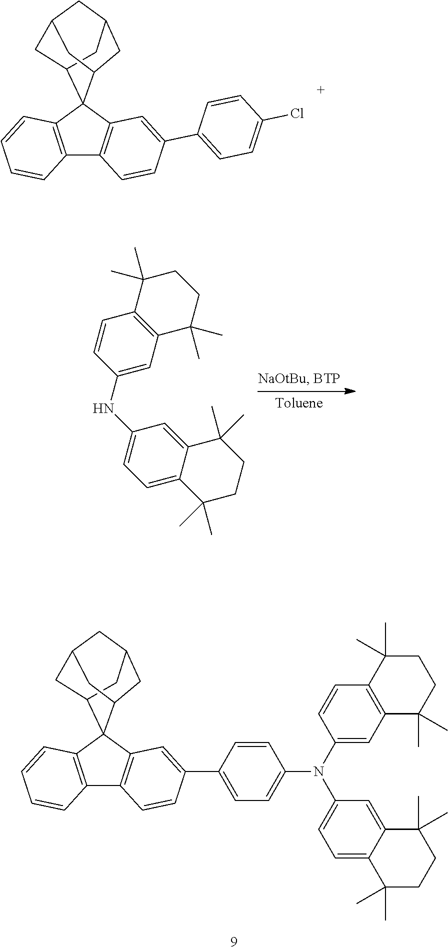 [SMILES: Clc1ccc(-c2ccc3c(c2)C2(c4ccccc4-3)C3CC4CC(C3)CC2C4)cc1 ; CC1(C)CCC(C)(C)c2cc(Nc3ccc4c(c3)C(C)(C)CCC4(C)C)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)C(C)(C)CCC4(C)C)ccc21]

Compound 9 (30.0 g, 79.38% yield) was obtained in the same manner as in Synthesis Example 1 using 2′-(4-chlorophenyl)spiro[adamanthane-2,9′-fluorene] (20.0 g, 50.38 mmol) and bis(5,5,8,8-tetramethyl-5,6,7,8-tetrahydronaphthalen-2-yl)amine (20.02 g, 51.39 mmol). (MS: [M+H]⁺=750)

### Synthesis Example 10. Synthesis of Compound 10

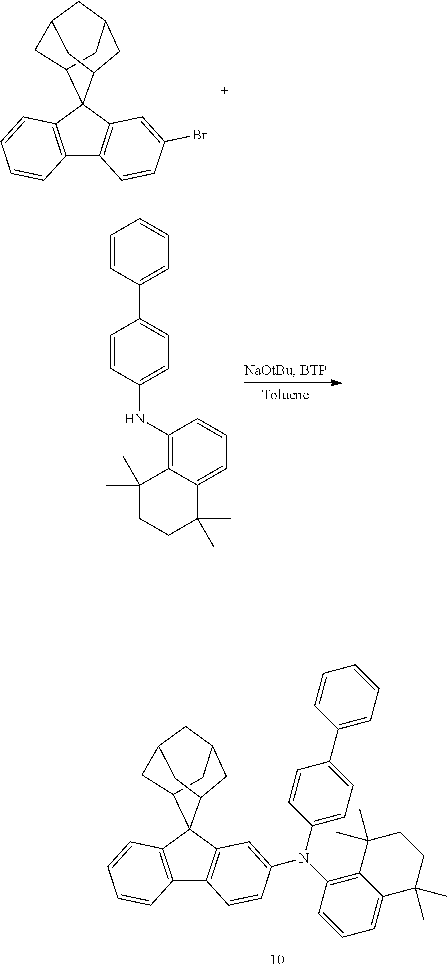

Compound 10 (27.5 g, 78.49% yield) was obtained in the same manner as in Synthesis Example 1 using 2′-bromospiro[adamanthane-2,9′-fluorene] (20.0 g, 54.75 mmol) and N-([1,1′-biphenyl]-4-yl)-5,5,8,8-tetramethyl-5,6,7,8-tetrahydronaphthalen-1-amine (19.85 g, 55.84 mmol). (MS: [M+H]⁺=640)

### Synthesis Example 11. Synthesis of Compound 11

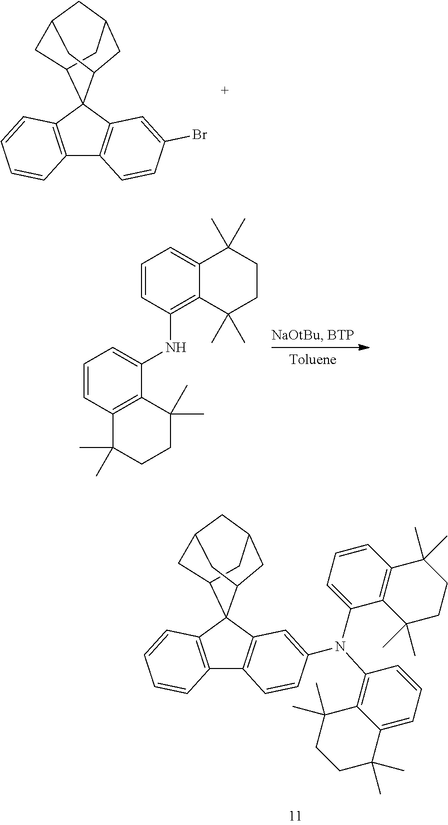 [SMILES: Brc1ccc2c(c1)C1(c3ccccc3-2)C2CC3CC(C2)CC1C3 ; CC1(C)CCC(C)(C)c2c(Nc3cccc4c3C(C)(C)CCC4(C)C)cccc21 ; CC1(C)CCC(C)(C)c2c(N(c3ccc4c(c3)C3(c5ccccc5-4)C4CC5CC(C4)CC3C5)c3cccc4c3C(C)(C)CCC4(C)C)cccc21]

Compound 11 (28.5 g, 77.23% yield) was obtained in the same manner as in Synthesis Example 1 using 2′-bromospiro[adamanthane-2,9′-fluorene] (20.0 g, 54.75 mmol) and bis(5,5,8,8-tetramethyl-5,6,7,8-tetrahydronaphthalen-1-yl)amine (21.76 g, 55.84 mmol). (MS: [M+H]⁺=674)

### Synthesis Example 12. Synthesis of Compound 12

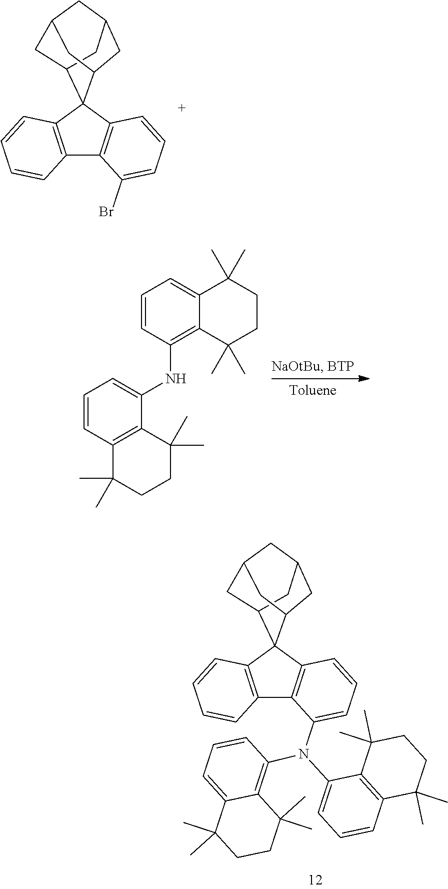

Compound 12 (28.5 g, 77.23% yield) was obtained in the same manner as in Synthesis Example 1 using 4′-bromospiro[adamanthane-2,9′-fluorene] (20.0 g, 54.75 mmol) and bis(5,5,8,8-tetramethyl-5,6,7,8-tetrahydronaphthalen-1-yl)amine (21.76 g, 55.84 mmol). (MS: [M+H]⁺=674)

### Synthesis Example 13. Synthesis of Compound 13

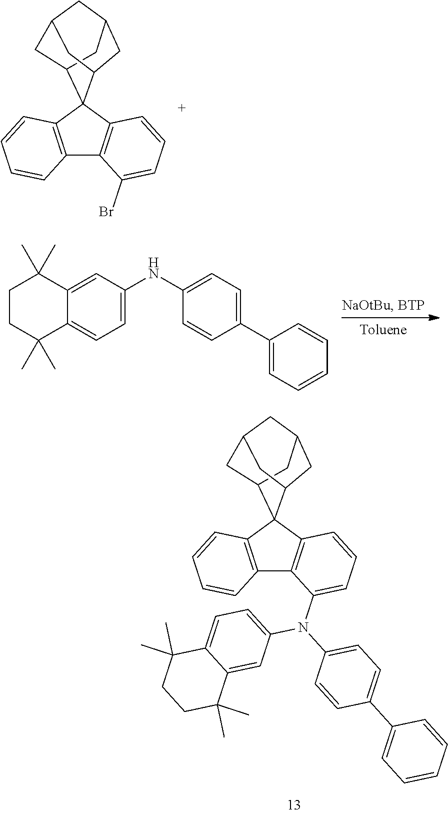

Compound 13 (27.5 g, 78.49% yield) was obtained in the same manner as in Synthesis Example 1 using 4′-bromospiro[adamanthane-2,9′-fluorene] (20.0 g, 54.75 mmol) and N-([1,1′-biphenyl]-4-yl)-5,5,8,8-tetramethyl-5,6,7,8-tetrahydronaphthalen-2-amine (19.85 g, 55.84 mmol). (MS: [M+H]⁺=640)

### Synthesis Example 14. Synthesis of Compound 14

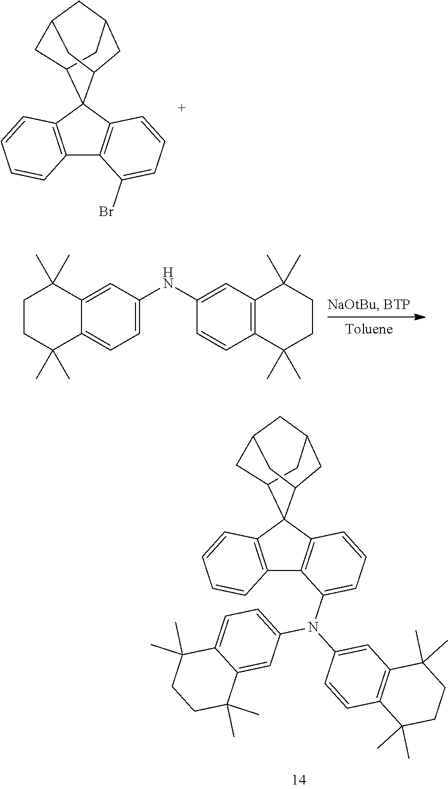

Compound 14 (29.0 g, 78.58% yield) was obtained in the same manner as in Synthesis Example 1 using 4′-bromospiro[adamanthane-2,9′-fluorene] (20.0 g, 54.75 mmol) and bis(5,5,8,8-tetramethyl-5,6,7,8-tetrahydronaphthalen-2-yl)amine (21.76 g, 55.84 mmol). (MS: [M+H]⁺=674)

### Synthesis Example 15. Synthesis of Compound 15

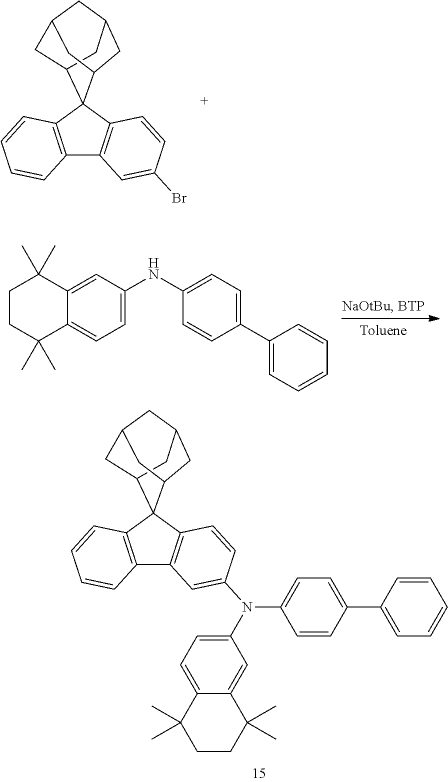 [SMILES: Brc1ccc2c(c1)-c1ccccc1C21C2CC3CC(C2)CC1C3 ; CC1(C)CCC(C)(C)c2cc(Nc3ccc(-c4ccccc4)cc3)ccc21 ; CC1(C)CCC(C)(C)c2cc(N(c3ccc(-c4ccccc4)cc3)c3ccc4c(c3)-c3ccccc3C43C4CC5CC(C4)CC3C5)ccc21]

Compound 15 (27.0 g, 77.06% yield) was obtained in the same manner as in Synthesis Example 1 using 3′-bromospiro[adamanthane-2,9′-fluorene] (20.0 g, 54.75 mmol) and N-([1,1′-biphenyl]-4-yl)-5,5,8,8-tetramethyl-5,6,7,8-tetrahydronaphthalen-2-amine (19.85 g, 55.84 mmol). (MS: [M+H]⁺=640)

### Synthesis Example 16. Synthesis of Compound 16

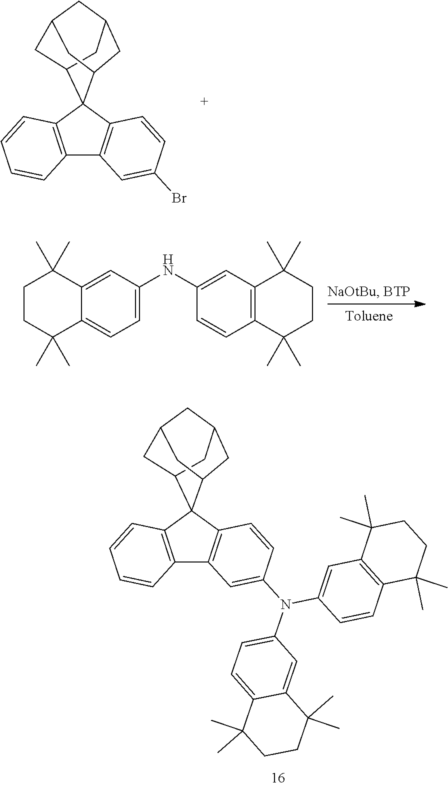 [SMILES: Brc1ccc2c(c1)-c1ccccc1C21C2CC3CC(C2)CC1C3 ; CC1(C)CCC(C)(C)c2cc(Nc3ccc4c(c3)C(C)(C)CCC4(C)C)ccc21 ; CC1(C)CCC(C)(C)c2cc(N(c3ccc4c(c3)-c3ccccc3C43C4CC5CC(C4)CC3C5)c3ccc4c(c3)C(C)(C)CCC4(C)C)ccc21]

Compound 16 (28.5 g, 77.23% yield) was obtained in the same manner as in Synthesis Example 1 using 3′-bromospiro[adamanthane-2,9′-fluorene] (20.0 g, 54.75 mmol) and bis(5,5,8,8-tetramethyl-5,6,7,8-tetrahydronaphthalen-2-yl)amine (21.76 g, 55.84 mmol). (MS: [M+H]⁺=674)

### Synthesis Example 17. Synthesis of Compound 17

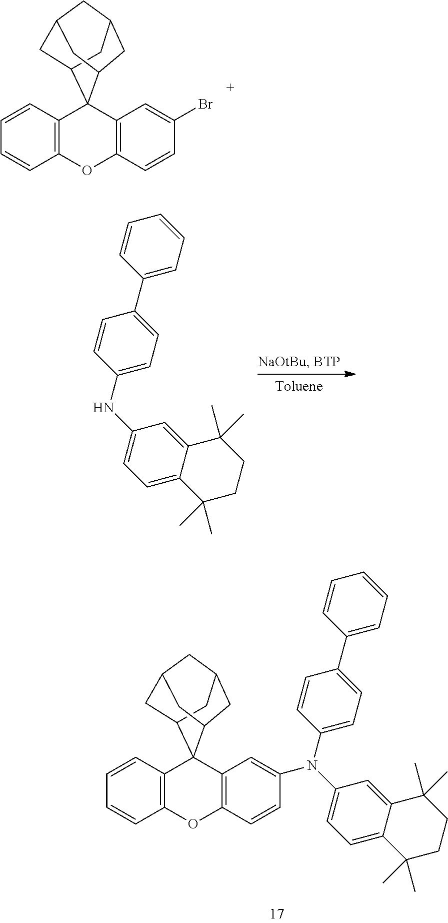

Compound 17 (27.0 g, 78.48% yield) was obtained in the same manner as in Synthesis Example 1 using 2′-bromospiro[adamanthane-2,9′-xanthene] (20.0 g, 52.45 mmol) and N-([1,1′-biphenyl]-4-yl)-5,5,8,8-tetramethyl-5,6,7,8-tetrahydronaphthalen-2-amine (19.02 g, 53.50 mmol). (MS: [M+H]⁺=656)

### Synthesis Example 18. Synthesis of Compound 18

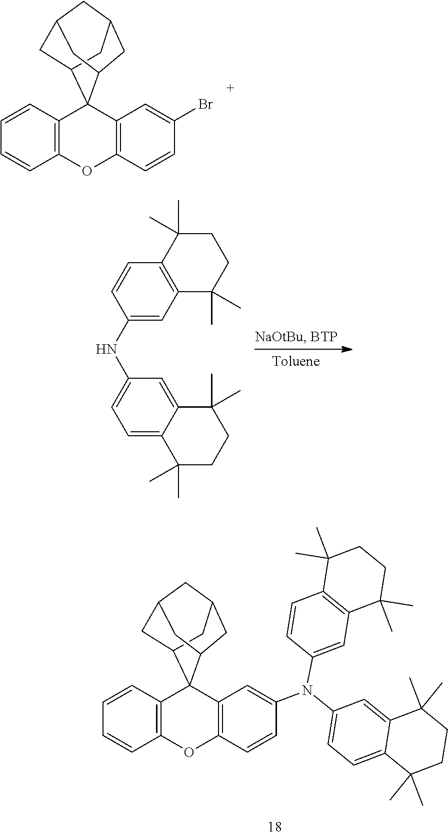

Compound 18 (28.5 g, 78.75% yield) was obtained in the same manner as in Synthesis Example 1 using 2′-bromospiro[adamanthane-2,9′-xanthene] (20.0 g, 52.45 mmol) and bis(5,5,8,8-tetramethyl-5,6,7,8-tetrahydronaphthalen-2-yl)amine (20.85 g, 53.50 mmol). (MS: [M+H]⁺=690)

### Synthesis Example 19. Synthesis of Compound 19

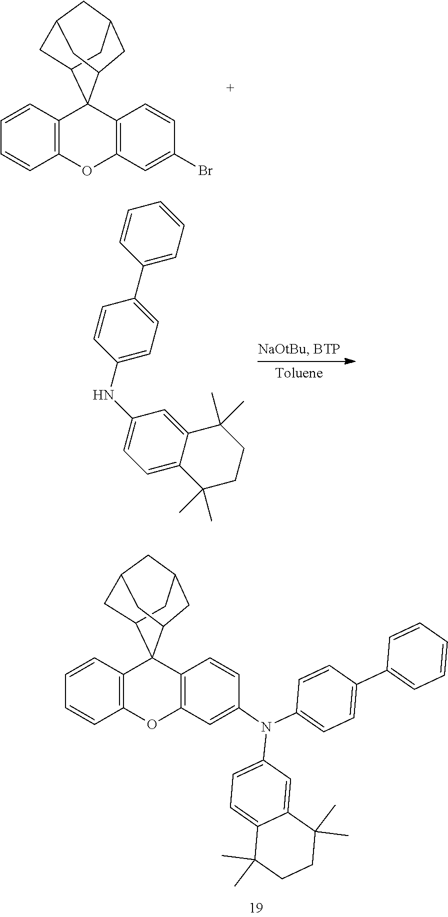

Compound 19 (27.0 g, 78.48% yield) was obtained in the same manner as in Synthesis Example 1 using 3′-bromospiro[adamanthane-2,9′-xanthene] (20.0 g, 52.45 mmol) and N-([1,1′-biphenyl]-4-yl)-5,5,8,8-tetramethyl-5,6,7,8-tetrahydronaphthalen-2-amine (19.02 g, 53.50 mmol). (MS: [M+H]⁺=656)

### Synthesis Example 20. Synthesis of Compound 20

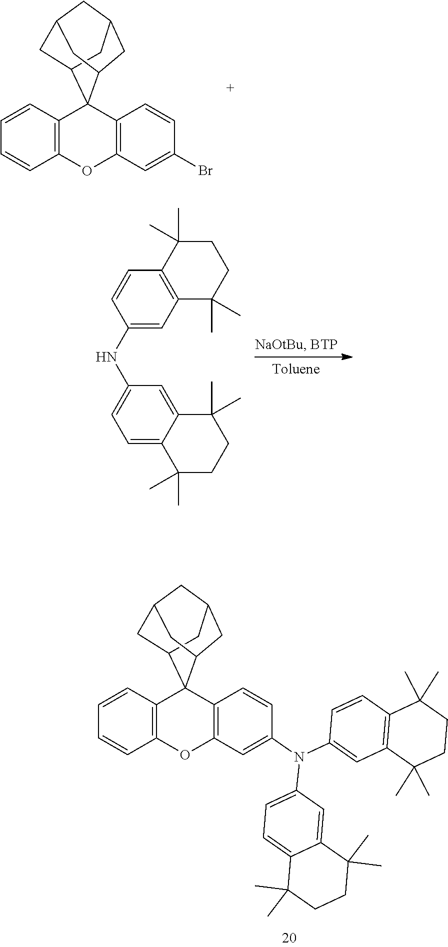

Compound 20 (28.5 g, 78.75% yield) was obtained in the same manner as in Synthesis Example 1 using 3′-bromospiro[adamanthane-2,9′-xanthene] (20.0 g, 52.45 mmol) and bis(5,5,8,8-tetramethyl-5,6,7,8-tetrahydronaphthalen-2-yl)amine (20.85 g, 53.50 mmol). (MS: [M+H]⁺=690)

### Synthesis Example 21. Synthesis of Compound 21

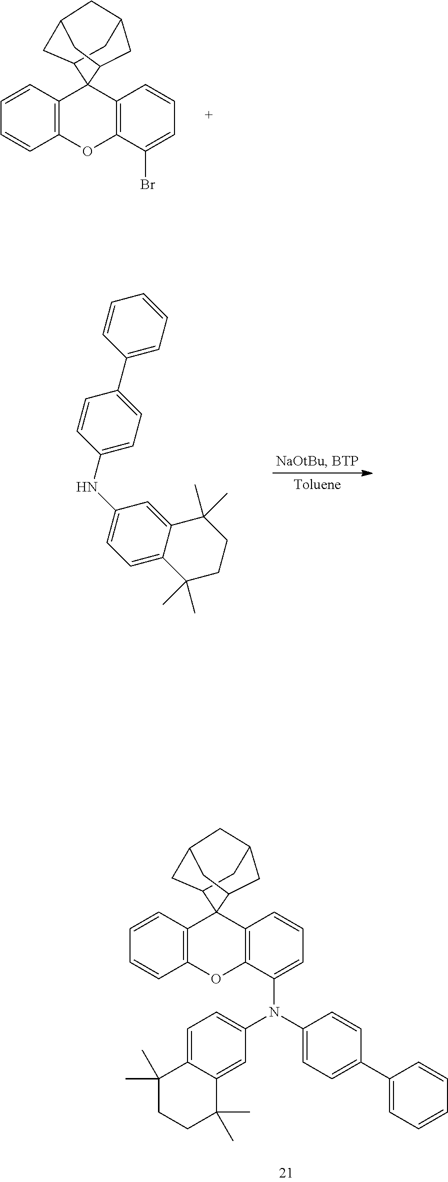 [SMILES: Brc1cccc2c1Oc1ccccc1C21C2CC3CC(C2)CC1C3 ; CC1(C)CCC(C)(C)c2cc(Nc3ccc(-c4ccccc4)cc3)ccc21 ; CC1(C)CCC(C)(C)c2cc(N(c3ccc(-c4ccccc4)cc3)c3cccc4c3Oc3ccccc3C43C4CC5CC(C4)CC3C5)ccc21]

Compound 21 (26.5 g, 77.03% yield) was obtained in the same manner as in Synthesis Example 1 using 4′-bromospiro[adamanthane-2,9′-xanthene] (20.0 g, 52.45 mmol) and N-([1,1′-biphenyl]-4-yl)-5,5,8,8-tetramethyl-5,6,7,8-tetrahydronaphthalen-2-amine (19.02 g, 53.50 mmol). (MS: [M+H]⁺=656)

### Synthesis Example 22. Synthesis of Compound 22

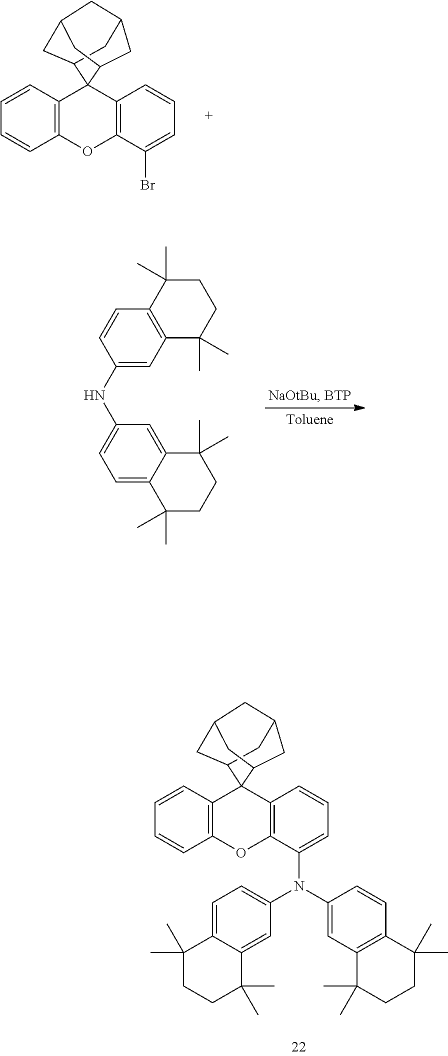

Compound 22 (28.0 g, 77.37% yield) was obtained in the same manner as in Synthesis Example 1 using 4′-bromospiro[adamanthane-2,9′-xanthene] (20.0 g, 52.45 mmol) and bis(5,5,8,8-tetramethyl-5,6,7,8-tetrahydronaphthalen-2-yl)amine (20.85 g, 53.50 mmol). (MS: [M+H]⁺=690)

### Synthesis Example 23. Synthesis of Compound 23

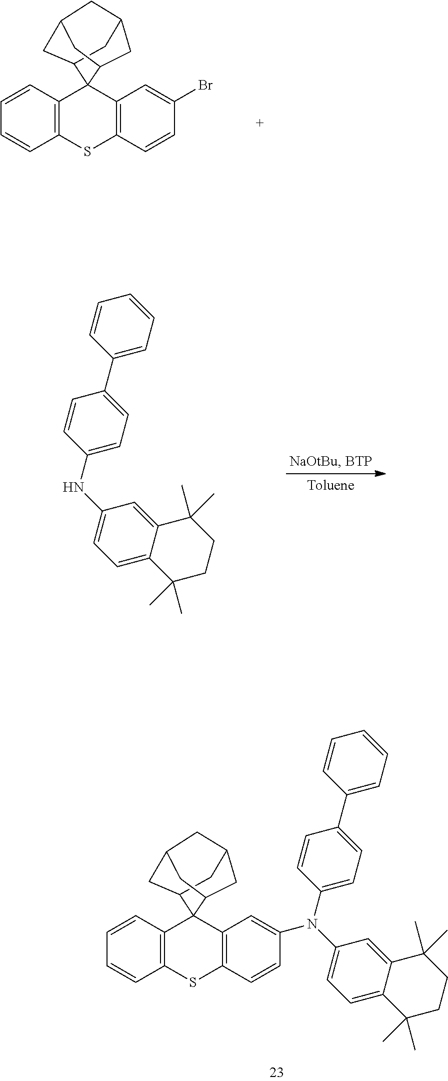 [SMILES: Brc1ccc2c(c1)C1(c3ccccc3S2)C2CC3CC(C2)CC1C3 ; CC1(C)CCC(C)(C)c2cc(Nc3ccc(-c4ccccc4)cc3)ccc21 ; CC1(C)CCC(C)(C)c2cc(N(c3ccc(-c4ccccc4)cc3)c3ccc4c(c3)C3(c5ccccc5S4)C4CC5CC(C4)CC3C5)ccc21]

Compound 23 (26.5 g, 78.35% yield) was obtained in the same manner as in Synthesis Example 1 using 2′-bromospiro[adamanthane-2,9′-thioxanthene] (20.0 g, 50.33 mmol) and N-([1,1′-biphenyl]-4-yl)-5,5,8,8-tetramethyl-5,6,7,8-tetrahydronaphthalen-2-amine (18.25 g, 51.34 mmol). (MS: [M+H]⁺=672)

### Synthesis Example 24. Synthesis of Compound 24

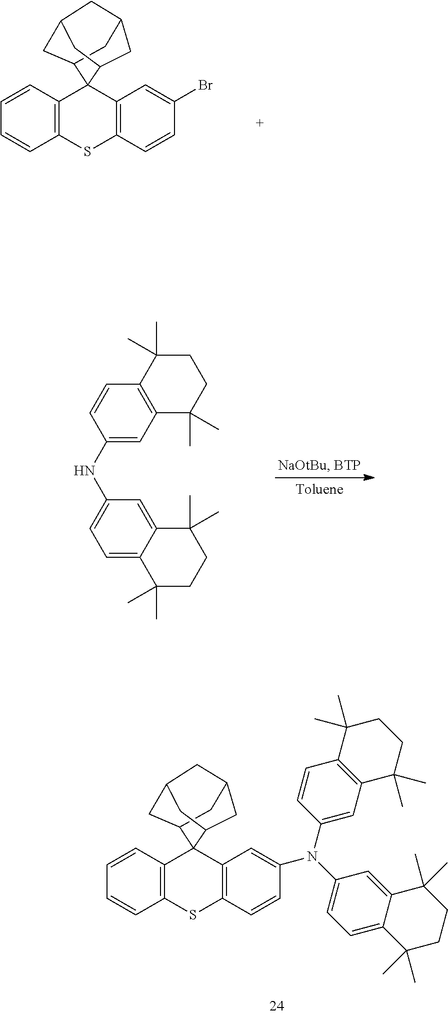 [SMILES: Brc1ccc2c(c1)C1(c3ccccc3S2)C2CC3CC(C2)CC1C3 ; CC1(C)CCC(C)(C)c2cc(Nc3ccc4c(c3)C(C)(C)CCC4(C)C)ccc21 ; CC1(C)CCC(C)(C)c2cc(N(c3ccc4c(c3)C(C)(C)CCC4(C)C)c3ccc4c(c3)C3(c5ccccc5S4)C4CC5CC(C4)CC3C5)ccc21]

Compound 24 (28.0 g, 78.79% yield) was obtained in the same manner as in Synthesis Example 1 using 2′-bromospiro[adamanthane-2,9′-thioxanthene] (20.0 g, 50.33 mmol) and bis(5,5,8,8-tetramethyl-5,6,7,8-tetrahydronaphthalen-2-yl)amine (20.00 g, 51.34 mmol). (MS: [M+H]⁺=706)

### Synthesis Example 25. Synthesis of Compound 25

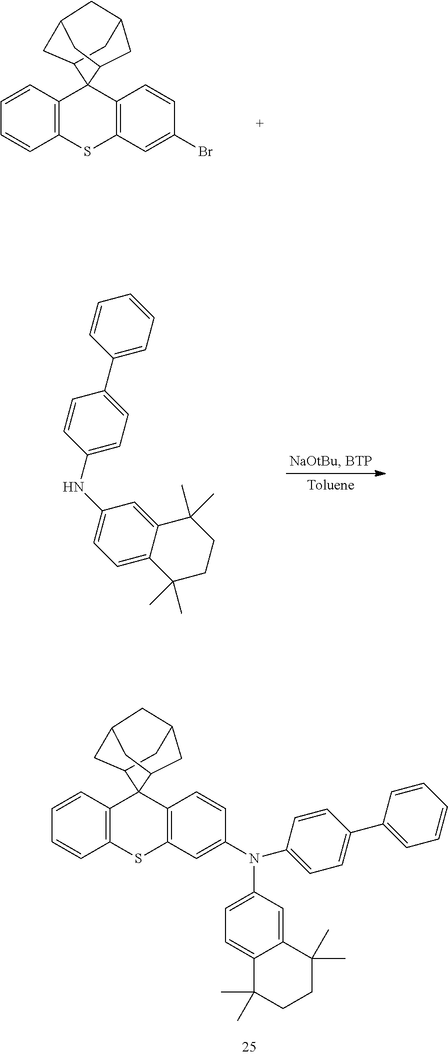 [SMILES: Brc1ccc2c(c1)Sc1ccccc1C21C2CC3CC(C2)CC1C3 ; CC1(C)CCC(C)(C)c2cc(Nc3ccc(-c4ccccc4)cc3)ccc21 ; CC1(C)CCC(C)(C)c2cc(N(c3ccc(-c4ccccc4)cc3)c3ccc4c(c3)Sc3ccccc3C43C4CC5CC(C4)CC3C5)ccc21]

Compound 25 (26.5 g, 78.35% yield) was obtained in the same manner as in Synthesis Example 1 using 3′-bromospiro[adamanthane-2,9′-thioxanthene] (20.0 g, 50.33 mmol) and N-([1,1′-biphenyl]-4-yl)-5,5,8,8-tetramethyl-5,6,7,8-tetrahydronaphthalen-2-amine (18.25 g, 51.34 mmol). (MS: [M+H]⁺=672)

### Synthesis Example 26. Synthesis of Compound 26

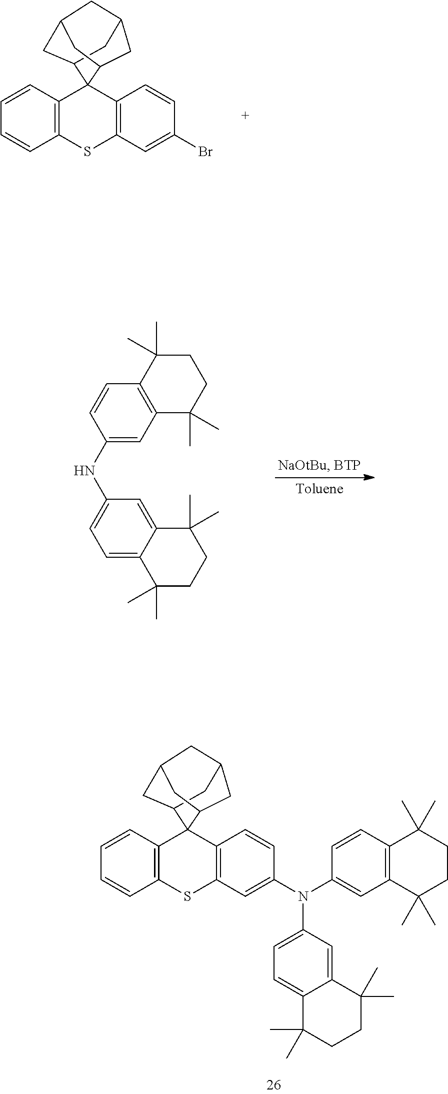 [SMILES: Brc1ccc2c(c1)Sc1ccccc1C21C2CC3CC(C2)CC1C3 ; CC1(C)CCC(C)(C)c2cc(Nc3ccc4c(c3)C(C)(C)CCC4(C)C)ccc21 ; CC1(C)CCC(C)(C)c2cc(N(c3ccc4c(c3)Sc3ccccc3C43C4CC5CC(C4)CC3C5)c3ccc4c(c3)C(C)(C)CCC4(C)C)ccc21]

Compound 26 (28.0 g, 78.79% yield) was obtained in the same manner as in Synthesis Example 1 using 3′-bromospiro[adamanthane-2,9′-thioxanthene] (20.0 g, 50.33 mmol) and bis(5,5,8,8-tetramethyl-5,6,7,8-tetrahydronaphthalen-2-yl)amine (20.00 g, 51.34 mmol). (MS: [M+H]⁺=706)

### Synthesis Example 27. Synthesis of Compound 27

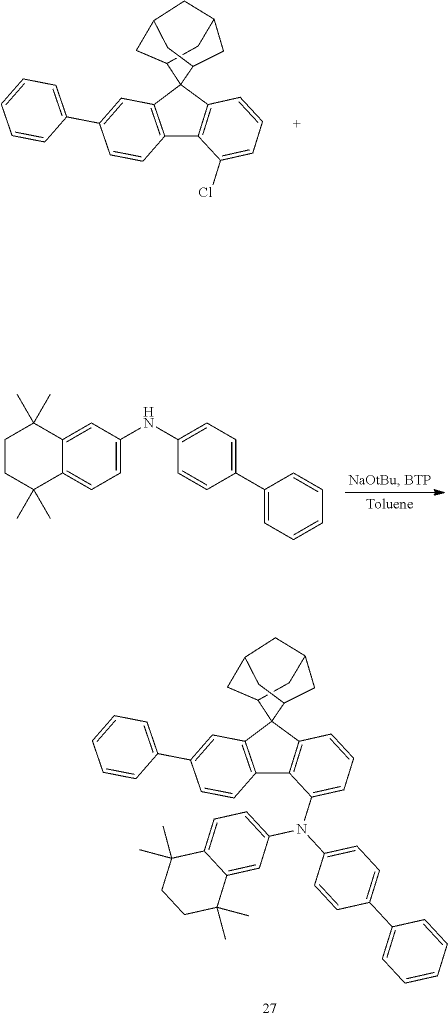 [SMILES: Clc1cccc2c1-c1ccc(-c3ccccc3)cc1C21C2CC3CC(C2)CC1C3 ; CC1(C)CCC(C)(C)c2cc(Nc3ccc(-c4ccccc4)cc3)ccc21 ; CC1(C)CCC(C)(C)c2cc(N(c3ccc(-c4ccccc4)cc3)c3cccc4c3-c3ccc(-c5ccccc5)cc3C43C4CC5CC(C4)CC3C5)ccc21]

Compound 27 (28.5 g, 79.01% yield) was obtained in the same manner as in Synthesis Example 1 using 5′-chloro-2′-phenylspiro[adamanthane-2,9′-fluorene] (20.0 g, 50.38 mmol) and N-([1,1′-biphenyl]-4-yl)-5,5,8,8-tetramethyl-5,6,7,8-tetrahydronaphthalen-2-amine (18.27 g, 51.39 mmol). (MS: [M+H]⁺=716)

### Synthesis Example 28. Synthesis of Compound 28

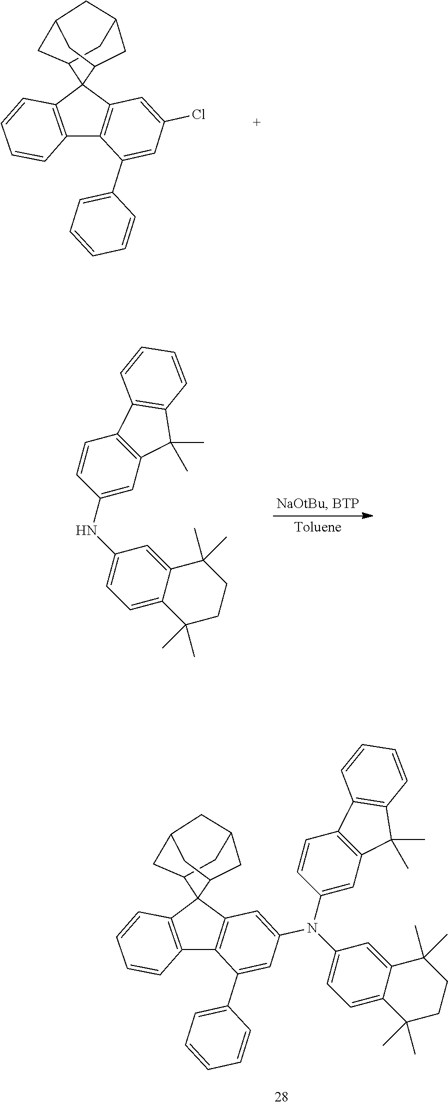 [SMILES: Clc1cc(-c2ccccc2)c2c(c1)C1(c3ccccc3-2)C2CC3CC(C2)CC1C3 ; CC1(C)CCC(C)(C)c2cc(Nc3ccc4c(c3)C(C)(C)c3ccccc3-4)ccc21 ; CC1(C)CCC(C)(C)c2cc(N(c3ccc4c(c3)C(C)(C)c3ccccc3-4)c3cc(-c4ccccc4)c4c(c3)C3(c5ccccc5-4)C4CC5CC(C4)CC3C5)ccc21]

Compound 28 (30.0 g, 78.76% yield) was obtained in the same manner as in Synthesis Example 1 using 2′-chloro-4′-phenylspiro[adamanthane-2,9′-fluorene] (20.0 g, 50.38 mmol) and 9,9-dimethyl-N-(5,5,8,8-tetramethyl-5,6,7,8-tetrahydronaphthalen-2-yl)-9H-fluoren-2-amine (20.33 g, 51.39 mmol). (MS: [M+H]⁺=756)

### Synthesis Example 29. Synthesis of Compound 29

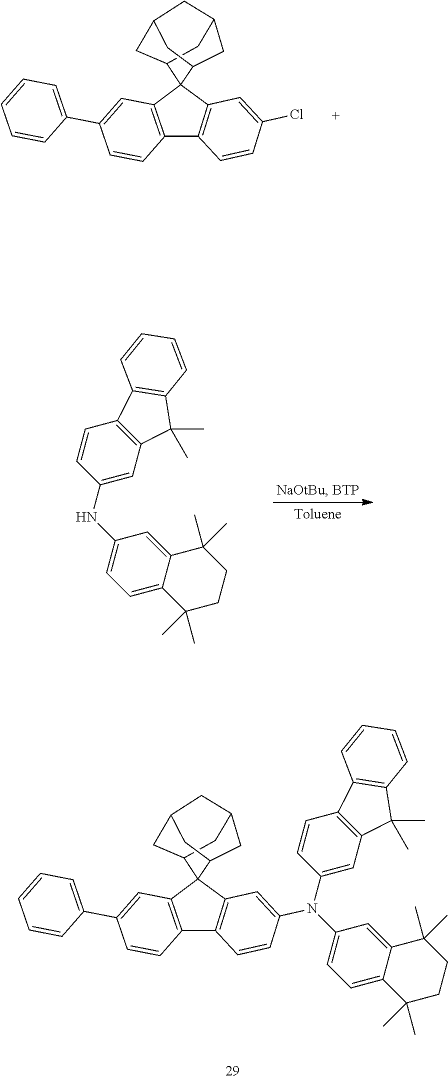 [SMILES: Clc1ccc2c(c1)C1(c3cc(-c4ccccc4)ccc3-2)C2CC3CC(C2)CC1C3 ; CC1(C)CCC(C)(C)c2cc(Nc3ccc4c(c3)C(C)(C)c3ccccc3-4)ccc21 ; CC1(C)CCC(C)(C)c2cc(N(c3ccc4c(c3)C(C)(C)c3ccccc3-4)c3ccc4c(c3)C3(c5cc(-c6ccccc6)ccc5-4)C4CC5CC(C4)CC3C5)ccc21]

Compound 29 (30.5 g, 80.01% yield) was obtained in the same manner as in Synthesis Example 1 using 2′-chloro-7′-phenylspiro[adamanthane-2,9′-fluorene] (20.0 g, 50.38 mmol) and 9,9-dimethyl-N-(5,5,8,8-tetramethyl-5,6,7,8-tetrahydronaphthalen-2-yl)-9H-fluoren-2-amine (20.33 g, 51.39 mmol). (MS: [M+H]⁺=756)

### Synthesis Example 30. Synthesis of Compound 30

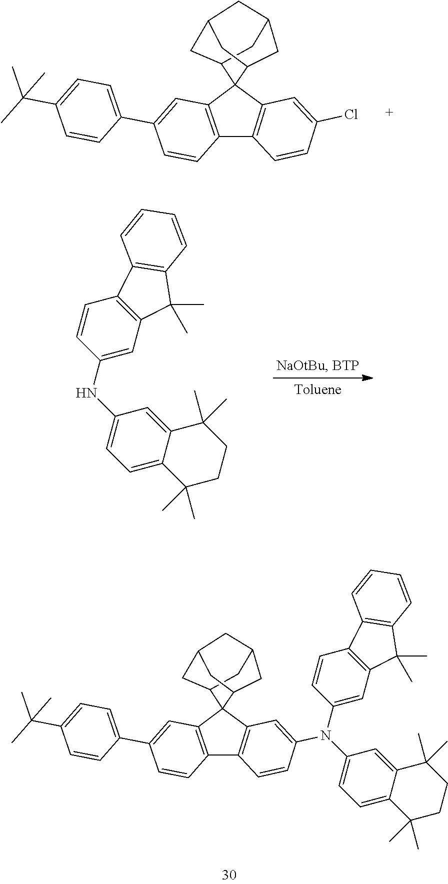 [SMILES: CC(C)(C)c1ccc(-c2ccc3c(c2)C2(c4cc(Cl)ccc4-3)C3CC4CC(C3)CC2C4)cc1 ; CC1(C)CCC(C)(C)c2cc(Nc3ccc4c(c3)C(C)(C)c3ccccc3-4)ccc21 ; CC(C)(C)c1ccc(-c2ccc3c(c2)C2(c4cc(N(c5ccc6c(c5)C(C)(C)CCC6(C)C)c5ccc6c(c5)C(C)(C)c5ccccc5-6)ccc4-3)C3CC4CC(C3)CC2C4)cc1]

Compound 30 (28.5 g, 79.50% yield) was obtained in the same manner as in Synthesis Example 1 using 2′-(4-(tert-butyl)phenyl)-7′-chlorospiro[adamanthane-2,9′-fluorene] (20.0 g, 44.14 mmol) and 9,9-dimethyl-N-(5,5,8,8-tetramethyl-5,6,7,8-tetrahydronaphthalen-2-yl)-9H-fluoren-2-amine (17.81 g, 45.03 mmol). (MS: [M+H]⁺=812)

### Synthesis Example 31. Synthesis of Compound 31

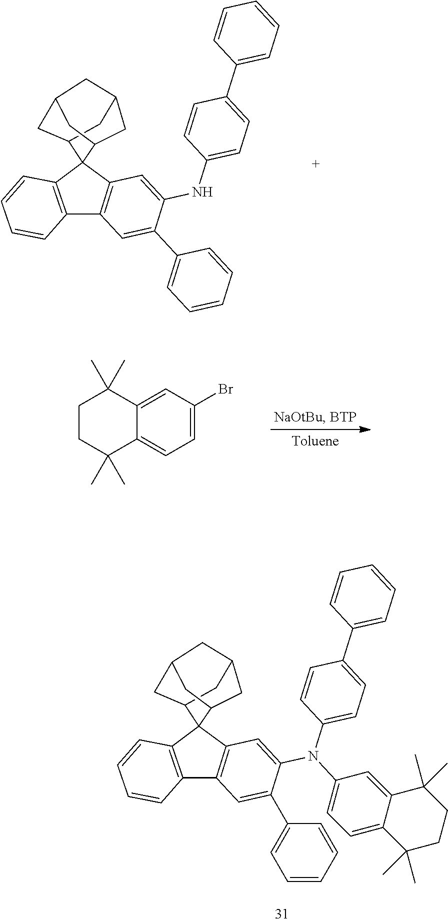 [SMILES: c1ccc(-c2ccc(Nc3cc4c(cc3-c3ccccc3)-c3ccccc3C43C4CC5CC(C4)CC3C5)cc2)cc1 ; CC1(C)CCC(C)(C)c2cc(Br)ccc21 ; CC1(C)CCC(C)(C)c2cc(N(c3ccc(-c4ccccc4)cc3)c3cc4c(cc3-c3ccccc3)-c3ccccc3C43C4CC5CC(C4)CC3C5)ccc21]

Compound 31 (21.0 g, 77.67% yield) was obtained in the same manner as in Synthesis Example 1 using N-([1,1′-biphenyl]-4-yl)-3′-phenylspiro[adamanthane-2,9′-fluorene]-2′-amine (20.0 g, 37.76 mmol) and 6-bromo-1,1,4,4-tetramethyl-1,2,3,4-tetrahydronaphthalene (10.29 g, 38.51 mmol). (MS: [M+H]⁺=716)

### Synthesis Example 32. Synthesis of Compound 32

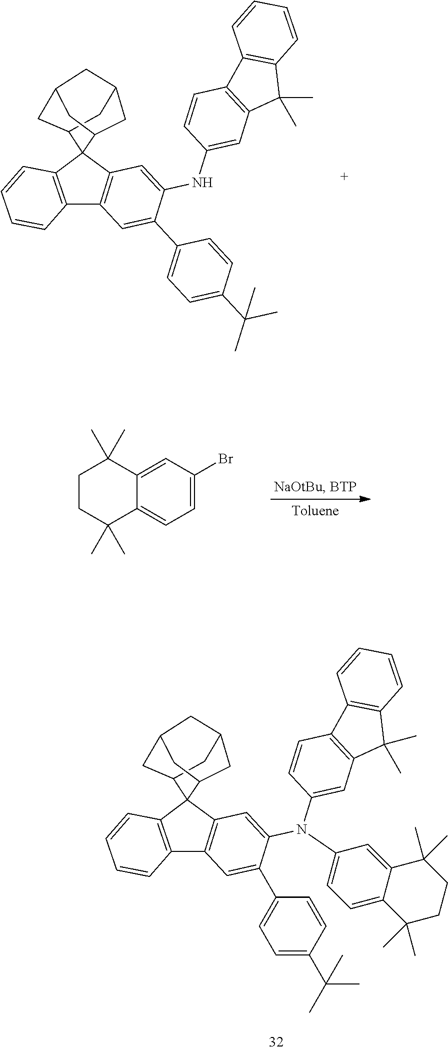 [SMILES: CC(C)(C)c1ccc(-c2cc3c(cc2Nc2ccc4c(c2)C(C)(C)c2ccccc2-4)C2(c4ccccc4-3)C3CC4CC(C3)CC2C4)cc1 ; CC1(C)CCC(C)(C)c2cc(Br)ccc21 ; CC(C)(C)c1ccc(-c2cc3c(cc2N(c2ccc4c(c2)C(C)(C)CCC4(C)C)c2ccc4c(c2)C(C)(C)c2ccccc2-4)C2(c4ccccc4-3)C3CC4CC(C3)CC2C4)cc1]

Compound 32 (20.0 g, 77.07% yield) was obtained in the same manner as in Synthesis Example 1 using 3′-(4-(tert-butyl)phenyl)-N-(9,9-dimethyl-9H-fluoren-2-yl)spiro[adamanthane-2,9′-fluorene]-2′-amine (20.0 g, 31.95 mmol) and 6-bromo-1,1,4,4-tetramethyl-1,2,3,4-tetrahydronaphthalene (8.71 g, 32.59 mmol). (MS: [M+H]⁺=812)

### Synthesis Example 33. Synthesis of Compound 33

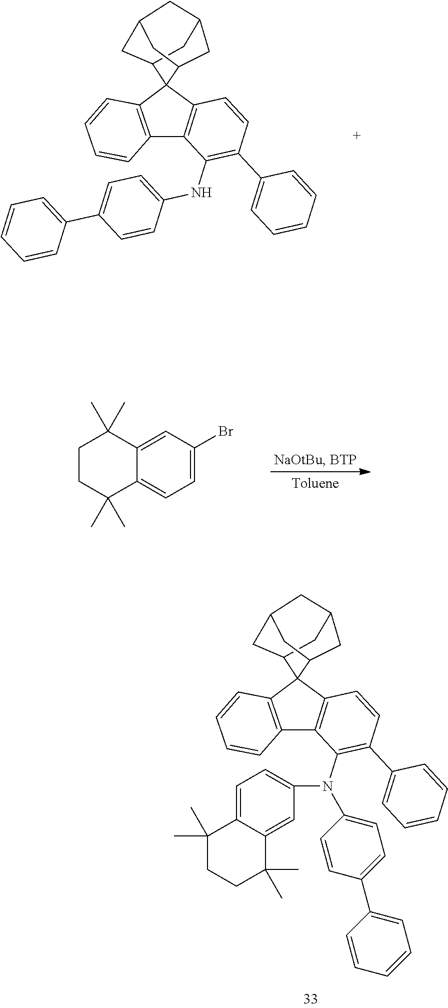 [SMILES: c1ccc(-c2ccc(Nc3c(-c4ccccc4)ccc4c3-c3ccccc3C43C4CC5CC(C4)CC3C5)cc2)cc1 ; CC1(C)CCC(C)(C)c2cc(Br)ccc21 ; CC1(C)CCC(C)(C)c2cc(N(c3ccc(-c4ccccc4)cc3)c3c(-c4ccccc4)ccc4c3-c3ccccc3C43C4CC5CC(C4)CC3C5)ccc21]

Compound 33 (20.5 g, 75.82% yield) was obtained in the same manner as in Synthesis Example 1 using N-([1,1′-biphenyl]-4-yl)-3′-phenylspiro[adamanthane-2,9′-fluorene]-4′-amine (20.0 g, 37.76 mmol) and 6-bromo-1,1,4,4-tetramethyl-1,2,3,4-tetrahydronaphthalene (10.29 g, 38.51 mmol). (MS: [M+H]⁺=716)

### Synthesis Example 34. Synthesis of Compound 34

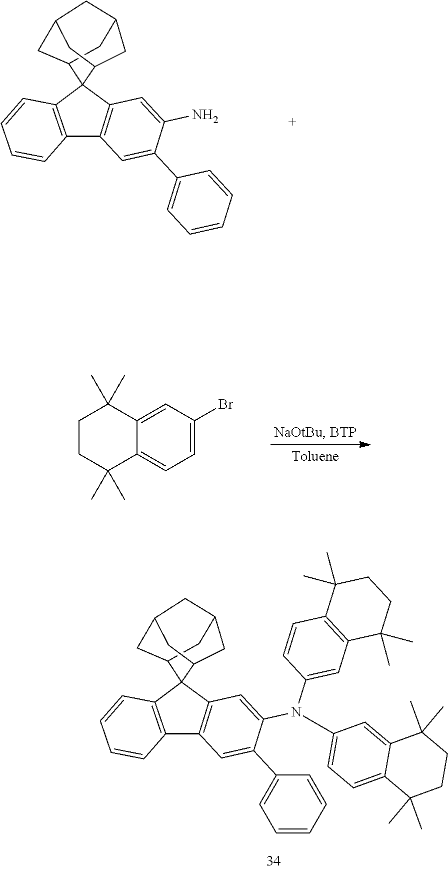 [SMILES: Nc1cc2c(cc1-c1ccccc1)-c1ccccc1C21C2CC3CC(C2)CC1C3 ; CC1(C)CCC(C)(C)c2cc(Br)ccc21 ; CC1(C)CCC(C)(C)c2cc(N(c3ccc4c(c3)C(C)(C)CCC4(C)C)c3cc4c(cc3-c3ccccc3)-c3ccccc3C43C4CC5CC(C4)CC3C5)ccc21]

3′-phenylspiro[adamanthane-2,9′-fluorene]-2′-amine (15.0 g, 39.73 mmol), 6-bromo-1,1,4,4-tetramethyl-1,2,3,4-tetrahydronaphthalene (21.76 g, 81.45 mmol), and sodium tert-butoxide (10.69 g, 111.24 mmol) were added to xylene (300 ml), followed by heating and stirring for 10 minutes. Bis(tri-tert-butylphosphine) palladium (0.10 g, 0.20 mmol) dissolved in xylene (30 ml) was added to the mixture, followed by heating and stirring for 1 hour. After completion of the reaction and filtration, layer separation was performed with xylene and water. After removal of the solvent, the compound 34 (23.5 g, 78.85% yield) was obtained by recrystallization with ethyl acetate. (MS: [M+H]⁺=750)

### EXAMPLES

### Example 1-1

A glass substrate on which ITO (Indium Tin Oxide) was coated as a thin film to a thickness of 1400 Å was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

The following compound HAT was thermally vacuum-deposited to a thickness of 100 Å on the prepared ITO transparent electrode to form a hole injection layer. On the hole injection layer, the following Compound HT1 was vacuum-deposited to a thickness of 1150 Å to form a hole transport layer, and the compound 1 prepared in Synthesis Example 1 was thermally vacuum-deposited to a thickness of 150 Å to form an electron blocking layer. Then, the following Compound BH and the following compound BD were vacuum-deposited at a weight ratio of 25:1 to a thickness of 200 Å to form a light emitting layer. Then, the following Compound HB1 was vacuum-deposited to a thickness of 50 Å to form a hole blocking layer. Then, the following compound ET1 and the following compound Liq were thermally vacuum-deposited at a weight ratio of 1:1 to a thickness of 310 Å to form an electron transport and injection layer. On the electron transport and injection layer, lithium fluoride (LiF) and aluminum were sequentially deposited to a thickness of 12 Å and 1000 Å, respectively, to form a cathode, thereby manufacturing an organic light emitting device.

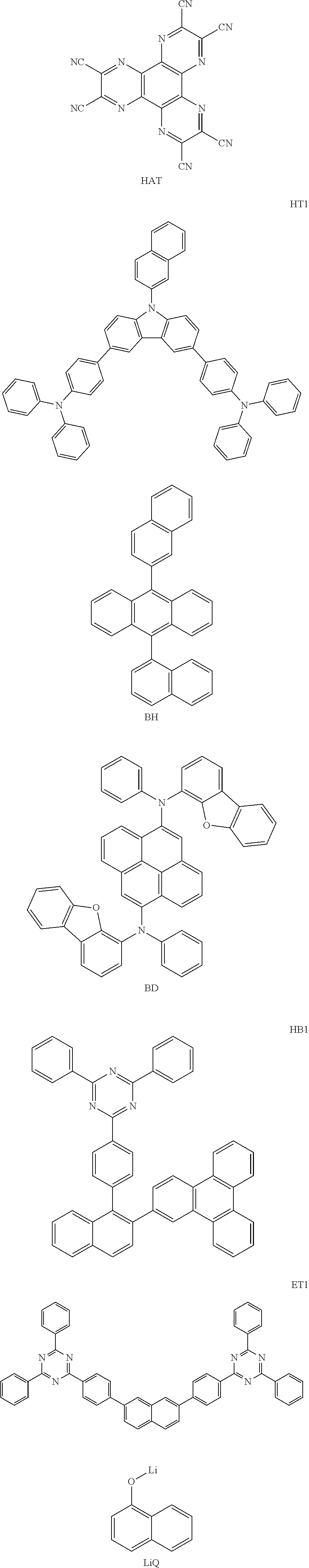

### Examples 1-2 to 1-20 and Comparative Examples 1-1 to 1-4

Organic light emitting devices of Examples 1-2 to 1-20 and Comparative Examples 1-1 to 1-4 were manufactured in the same manner as in Example 1-1, except that the compound shown in Table 1 was used instead of the compound 1. The compounds EB1 to EB4 used in Comparative Examples 1-1 to 1-4 are as follows.

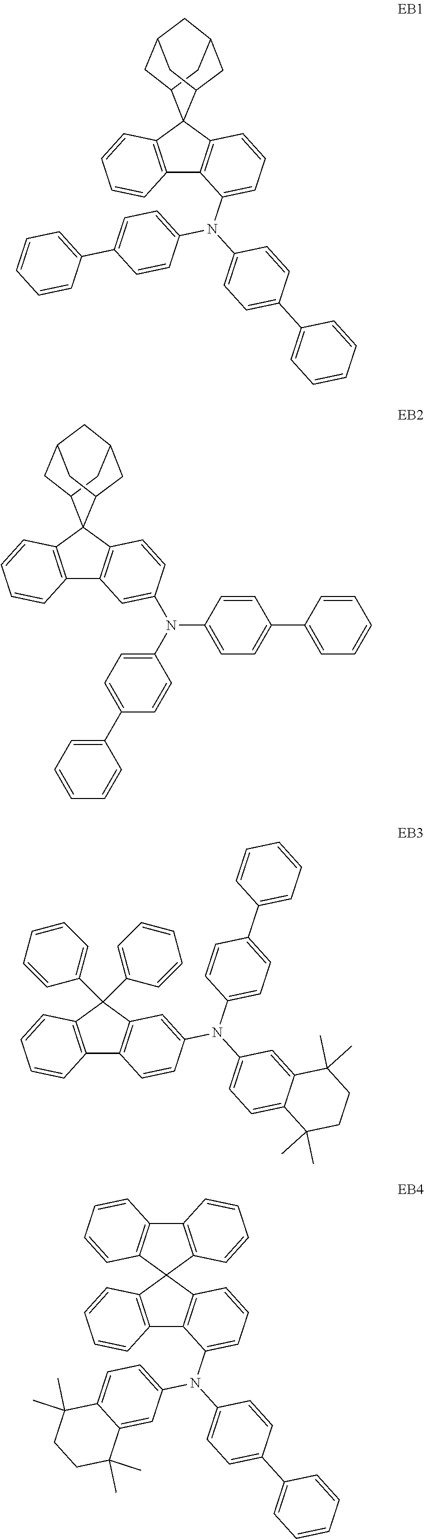

### Experimental Example 1

For the organic light emitting devices prepared in Examples 1-1 to 1-20 and Comparative Examples 1-1 to 1-4, the voltage, efficiency, color coordinate, and lifespan were measured by applying a current of 10 mA/cm², and the results are shown in Table 1. Meanwhile, T95 means the time taken until the initial luminance (6,000 nit) decreases to 95%.

TABLE 1

| | Electron | Voltage | Efficiency | Color | |
|---|---|---|---|---|---|
| | blocking | (V, @ | (cd/A, @ | coordinate | Lifespan |
| | layer | 10 mA/cm²) | 10 mA/cm²) | (x, y) | (T95, hr) |
| Example 1-1 | Compound | 3.68 | 5.93 | 0.141, 0.043 | 180 |
| | 1 | | | | |
| Example 1-2 | Compound | 3.67 | 5.94 | 0.140, 0.043 | 180 |
| | 2 | | | | |
| Example 1-3 | Compound | 3.68 | 5.92 | 0.141, 0.044 | 185 |
| | 5 | | | | |
| Example 1-4 | Compound | 3.66 | 5.95 | 0.140, 0.043 | 190 |
| | 6 | | | | |
| Example 1-5 | Compound | 3.67 | 5.94 | 0.140, 0.043 | 185 |
| | 7 | | | | |
| Example 1-6 | Compound | 3.68 | 5.91 | 0.140, 0.043 | 185 |
| | 8 | | | | |
| Example 1-7 | Compound | 3.69 | 5.94 | 0.140, 0.044 | 180 |
| | 9 | | | | |
| Example 1-8 | Compound | 3.72 | 5.97 | 0.141, 0.043 | 185 |
| | 12 | | | | |
| Example 1-9 | Compound | 3.71 | 5.96 | 0.140, 0.043 | 185 |
| | 13 | | | | |
| Example 1-10 | Compound | 3.71 | 5.98 | 0.140, 0.044 | 190 |
| | 14 | | | | |
| Example 1-11 | Compound | 3.70 | 5.95 | 0.140, 0.043 | 180 |
| | 15 | | | | |
| Example 1-12 | Compound | 3.68 | 5.96 | 0.140, 0.043 | 185 |
| | 16 | | | | |
| Example 1-13 | Compound | 3.70 | 5.95 | 0.140, 0.043 | 180 |
| | 19 | | | | |
| Example 1-14 | Compound | 3.71 | 5.96 | 0.140, 0.043 | 185 |
| | 21 | | | | |
| Example 1-15 | Compound | 3.67 | 5.97 | 0.140, 0.043 | 190 |
| | 22 | | | | |
| Example 1-16 | Compound | 3.71 | 5.95 | 0.140, 0.043 | 185 |
| | 25 | | | | |
| Example 1-17 | Compound | 3.72 | 5.96 | 0.140, 0.044 | 190 |
| | 27 | | | | |
| Example 1-18 | Compound | 3.69 | 5.95 | 0.141, 0.043 | 190 |
| | 31 | | | | |
| Example 1-19 | Compound | 3.67 | 5.96 | 0.140, 0.043 | 185 |
| | 32 | | | | |
| Example 1-20 | Compound | 3.68 | 5.94 | 0.141, 0.044 | 185 |
| | 33 | | | | |
| Comparative | Compound | 4.12 | 5.48 | 0.144, 0.048 | 130 |
| Example 1-1 | EB1 | | | | |
| Comparative | Compound | 4.00 | 5.35 | 0.145, 0.049 | 125 |
| Example 1-2 | EB2 | | | | |
| Comparative | Compound | 3.90 | 5.19 | 0.145, 0.048 | 110 |
| Example 1-3 | EB3 | | | | |
| Comparative | Compound | 4.05 | 5.26 | 0.145, 0.049 | 105 |
| Example 1-4 | EB4 | | | | |

As shown in Table 1, the compound of the present disclosure had excellent electron suppression ability, and thus the organic light emitting devices using it as the electron blocking layer were confirmed to have significant effects on driving voltage, efficiency, and lifespan.

### Examples 2-1 to 2-27 and Comparative Examples 2-1 to 2-5

Organic light emitting devices of Examples 2-1 to 2-27 and Comparative Examples 2-1 to 2-5 were manufactured in the same manner as in Example 1-1, except that the compound EB1 was used as the electron blocking layer instead of Compound 1, and the compound listed in Table 2 below was used as the hole transport layer instead of the compound HT1. The compounds HT2 to HT6 used in Comparative Examples 2-1 to 2-5 are as follows.

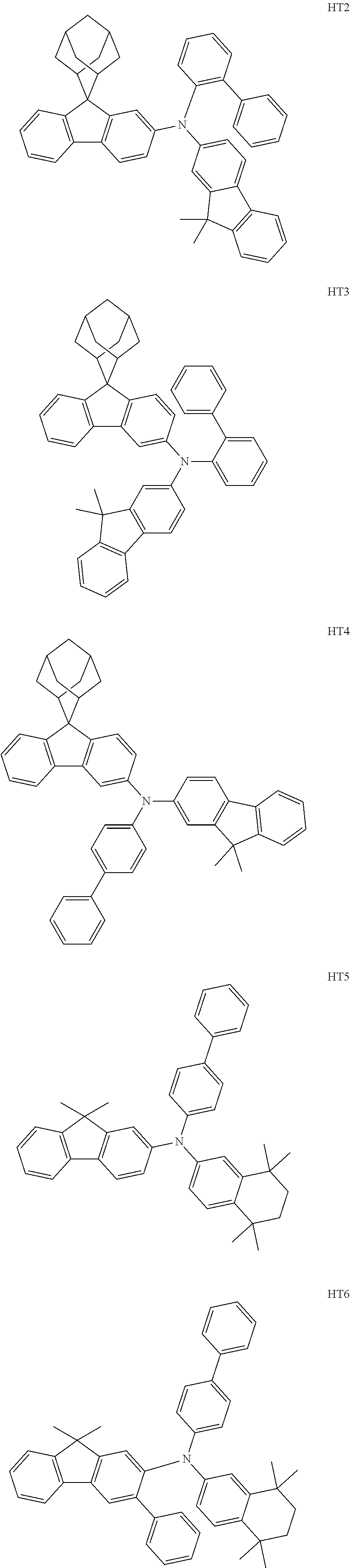 [SMILES: CC1(C)c2ccccc2-c2ccc(N(c3ccc4c(c3)C3(c5ccccc5-4)C4CC5CC(C4)CC3C5)c3ccccc3-c3ccccc3)cc21 ; CC1(C)c2ccccc2-c2ccc(N(c3ccc4c(c3)-c3ccccc3C43C4CC5CC(C4)CC3C5)c3ccccc3-c3ccccc3)cc21 ; CC1(C)c2ccccc2-c2ccc(N(c3ccc(-c4ccccc4)cc3)c3ccc4c(c3)-c3ccccc3C43C4CC5CC(C4)CC3C5)cc21 ; CC1(C)CCC(C)(C)c2cc(N(c3ccc(-c4ccccc4)cc3)c3ccc4c(c3)C(C)(C)c3ccccc3-4)ccc21 ; CC1(C)CCC(C)(C)c2cc(N(c3ccc(-c4ccccc4)cc3)c3cc4c(cc3-c3ccccc3)-c3ccccc3C4(C)C)ccc21]

### Experimental Example 2

For the organic light emitting devices prepared in Examples 2-1 to 2-27 and Comparative Examples 1-1, 2-1 to 2-5, the voltage, efficiency, color coordinate, and lifespan were measured by applying a current of 10 mA/cm², and the results are shown In Table 2. Meanwhile, T95 means the time taken until the initial luminance (6,000 nit) decreases to 95%.

TABLE 2

| | | Voltage | Efficiency | Color | |
|---|---|---|---|---|---|
| | Hole transport | (V, @ | (cd/A, @ | coordinate | Lifespan |
| | layer | 10 mA/cm²) | 10 mA/cm²) | (x, y) | (T95, hr) |
| Example 2-1 | Compound 1 | 3.62 | 5.96 | 0.140, 0.044 | 185 |
| Example 2-2 | Compound 3 | 3.61 | 5.97 | 0.141, 0.043 | 185 |
| Example 2-3 | Compound 4 | 3.63 | 5.95 | 0.141, 0.043 | 185 |
| Example 2-4 | Compound 6 | 3.60 | 5.97 | 0.140, 0.043 | 190 |
| Example 2-5 | Compound 7 | 3.62 | 5.95 | 0.140, 0.043 | 185 |
| Example 2-6 | Compound 8 | 3.61 | 5.96 | 0.140, 0.043 | 180 |
| Example 2-7 | Compound 9 | 3.63 | 5.94 | 0.140, 0.043 | 185 |
| Example 2-8 | Compound 10 | 3.61 | 5.94 | 0.140, 0.043 | 185 |
| Example 2-9 | Compound 11 | 3.62 | 5.96 | 0.140, 0.043 | 185 |
| Example 2-10 | Compound 12 | 3.68 | 5.99 | 0.140, 0.043 | 190 |
| Example 2-11 | Compound 14 | 3.67 | 5.98 | 0.141, 0.044 | 185 |
| Example 2-12 | Compound 16 | 3.65 | 5.96 | 0.140, 0.043 | 190 |
| Example 2-13 | Compound 17 | 3.62 | 5.96 | 0.140, 0.043 | 190 |
| Example 2-14 | Compound 18 | 3.62 | 5.97 | 0.140, 0.043 | 190 |
| Example 2-15 | Compound 20 | 3.69 | 5.98 | 0.140, 0.043 | 185 |
| Example 2-16 | Compound 22 | 3.69 | 5.98 | 0.140, 0.044 | 190 |
| Example 2-17 | Compound 23 | 3.63 | 5.97 | 0.140, 0.044 | 185 |
| Example 2-18 | Compound 24 | 3.61 | 5.97 | 0.140, 0.043 | 185 |
| Example 2-19 | Compound 25 | 3.66 | 5.97 | 0.140, 0.043 | 180 |
| Example 2-20 | Compound 26 | 3.64 | 5.98 | 0.140, 0.044 | 190 |
| Example 2-21 | Compound 28 | 3.63 | 5.96 | 0.140, 0.043 | 185 |
| Example 2-22 | Compound 29 | 3.62 | 5.94 | 0.140, 0.043 | 185 |
| Example 2-23 | Compound 30 | 3.61 | 5.96 | 0.140, 0.043 | 180 |
| Example 2-24 | Compound 31 | 3.63 | 5.94 | 0.140, 0.043 | 185 |
| Example 2-25 | Compound 32 | 3.62 | 5.98 | 0.140, 0.044 | 190 |
| Example 2-26 | Compound 33 | 3.68 | 5.97 | 0.141, 0.044 | 180 |
| Example 2-27 | Compound 34 | 3.62 | 5.98 | 0.140, 0.043 | 185 |
| Comparative | Compound | 4.12 | 5.48 | 0.144, 0.048 | 130 |
| Example 1-1 | HT1 | | | | |
| Comparative | Compound | 3.90 | 5.40 | 0.144, 0.049 | 135 |
| Example 2-1 | HT2 | | | | |
| Comparative | Compound | 3.95 | 5.46 | 0.144, 0.048 | 120 |
| Example 2-2 | HT3 | | | | |
| Comparative | Compound | 3.92 | 5.38 | 0.144, 0.048 | 115 |
| Example 2-3 | HT4 | | | | |
| Comparative | Compound | 3.85 | 5.25 | 0.144, 0.049 | 125 |
| Example 2-4 | HT5 | | | | |
| Comparative | Compound | 3.84 | 5.33 | 0.144, 0.049 | 135 |
| Example 2-5 | HT6 | | | | |

As shown in Table 2, the compound of the present disclosure had excellent hole transport ability, and thus the organic light emitting devices using it as the hole transport layer were confirmed to have significant effects on driving voltage, efficiency, and lifespan.

[DESCRIPTION OF SYMBOLS]

| | |
|---|---|
| 1: Substrate | 2: Anode |
| 3: Light emitting layer | 4: Cathode |
| 5: Hole injection layer | 6: Hole transport layer |
| 7: Electron blocking layer | 8: Electron injection and transport layer |

1. A compound represented by the following Chemical Formula 1: 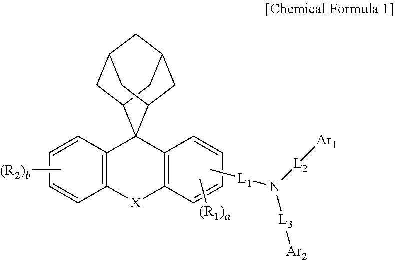
in Chemical Formula 1,
X is a single bond, O, or S,
R₁ and R₂ are each independently, hydrogen; deuterium; a halogen group; a nitrile group; a silyl group; a substituted or unsubstituted C₆₋₆₀ aryl; or a substituted or unsubstituted C₂₋₆₀ heteroaryl containing one or more selected from the group consisting of N, O, and S,
a is an integer of 0 to 3,
b is an integer of 0 to 4,
L₁ to L₃ are each independently a single bond; a substituted or unsubstituted C₆₋₆₀ arylene; or a substituted or unsubstituted C₂₋₆₀ heteroarylene containing one or more selected from the group consisting of N, O, and S, and
Ar₁ and Ar₂ are each independently a substituted or unsubstituted C₆₋₆₀ aryl; or a substituted or unsubstituted C₂₋₆₀ heteroaryl containing one or more selected from the group consisting of N, O, and S; wherein at least one of Ar₁ and Ar₂ is a substituent represented by Chemical Formula 2 below; 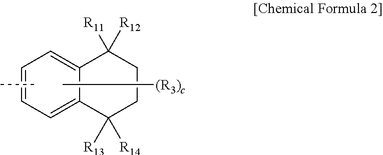
in Chemical Formula 2,
R₃ is deuterium; a halogen group; a nitrile group; a silyl group; a substituted or unsubstituted C₆₋₆₀ aryl; or a substituted or unsubstituted C₂₋₆₀ heteroaryl containing one or more selected from the group consisting of N, O, and S,
R₁₁ to R₁₄ are each independently —CH₃, —CH₂D, —CHD₂, or —CD₃,
c is an integer of 0 to 7, and
the silyl group is —Si(Z₁)(Z₂)(Z₃), wherein Z₁ to Z₃ are each independently a substituted or unsubstituted C₁₋₆₀ alkyl; or a substituted or unsubstituted C₆₋₆₀ aryl.

2. The compound of claim 1, wherein
R₁ and R₂ are each independently hydrogen, deuterium, phenyl, phenyl substituted with 1 or 2 tert-butyl, biphenylyl, naphthyl, or 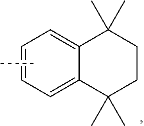
when R₁ and R₂ are each phenyl, phenyl substituted with 1 or 2 tert-butyl, biphenylyl, naphthyl, 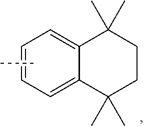
or wherein the phenyl, phenyl substituted with 1 or 2 tert-butyl, biphenylyl, naphthyl, or 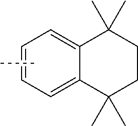
is unsubstituted or substituted with one or more deuterium.

3. The compound of claim 1, wherein
L₁ to L₃ are each independently, a single bond, phenylene, or phenylene substituted with 4 deuterium.

4. The compound of claim 1, wherein
Ar₁ and Ar₂ are each independently, phenyl, phenyl substituted with 1 tert-butyl, phenyl substituted with 1 adamantyl, biphenylyl, terphenylyl, naphthyl, phenyl naphthyl, naphthyl phenyl, dimethylfluorenyl, phenylfluorenyl, diphenylfluorenyl, carbazolyl, dibenzofuranyl, dibenzothiophenyl, or 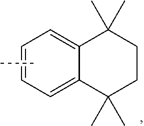
wherein Ar₁ and Ar₂ are unsubstituted or substituted with one or more deuterium, and
at least one of Ar₁ and Ar₂ is 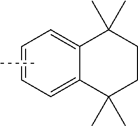
which is unsubstituted or substituted with one or more deuterium.

5. The compound of claim 1, wherein
the substituent represented by Chemical Formula 2 is any one selected from the group consisting of:

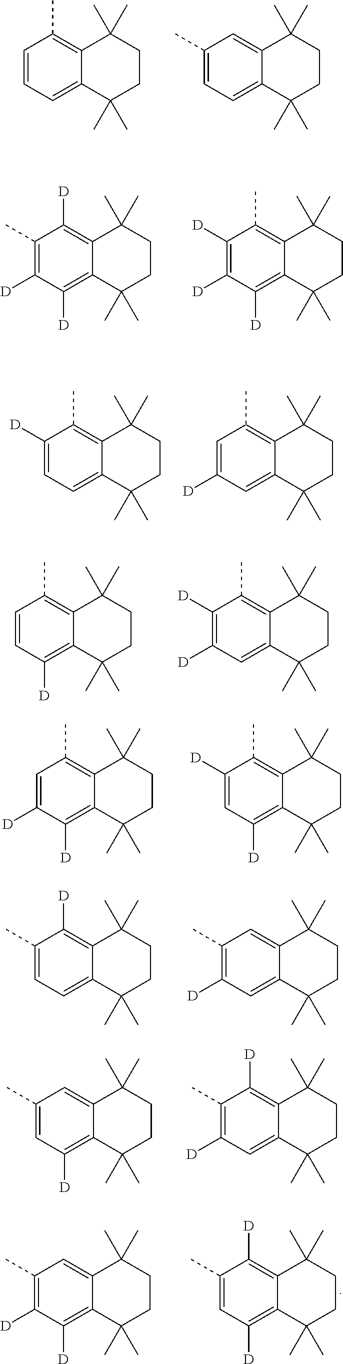

6. The compound of claim 1, wherein
Ar₁ and Ar₂ are each independently any one selected from the group consisting of the following, and
at least one of Ar₁ and Ar₂ is

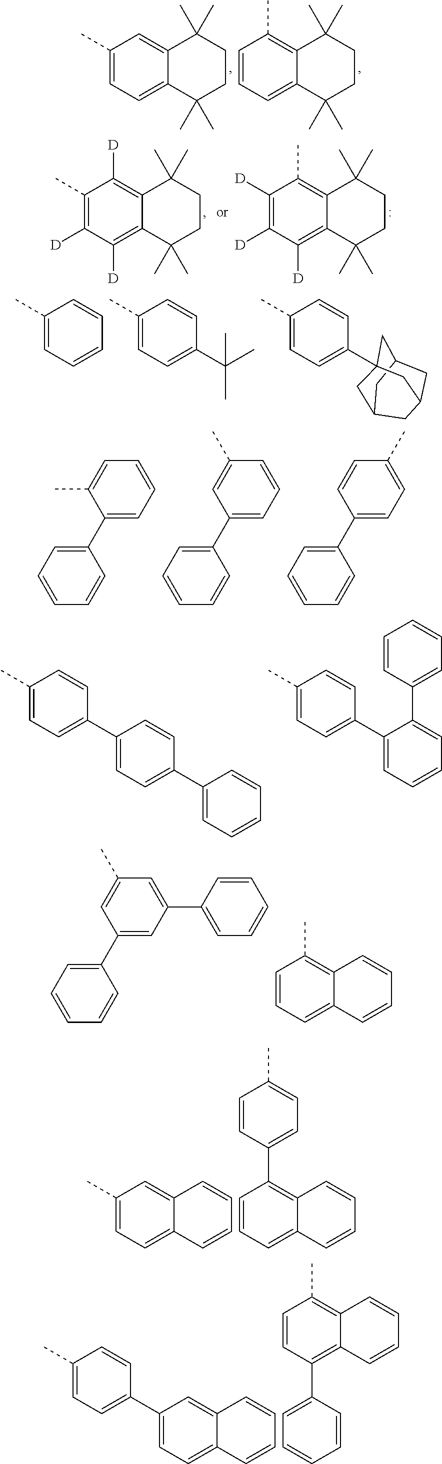

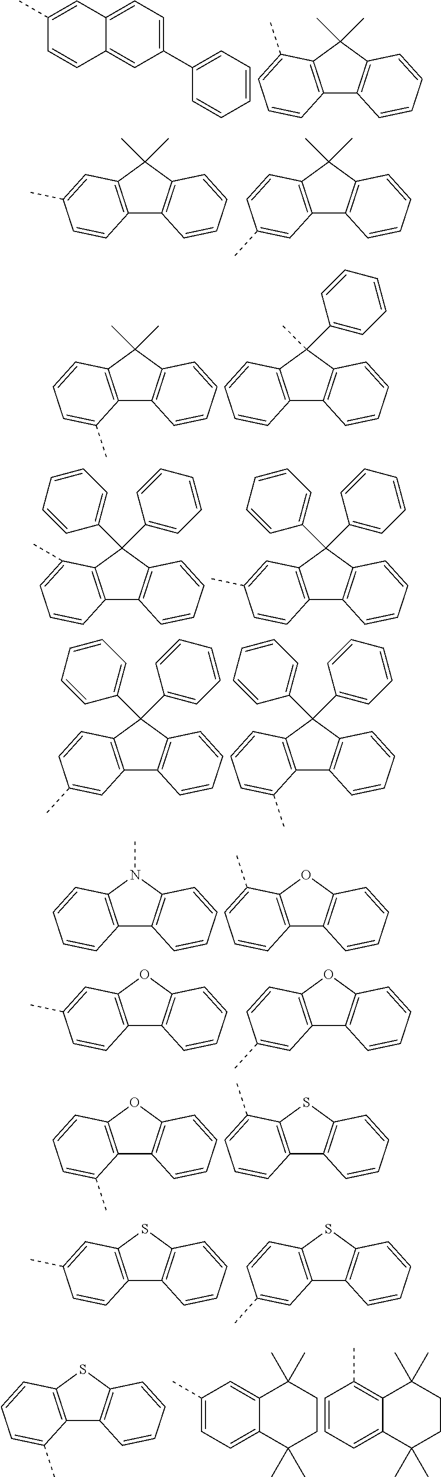

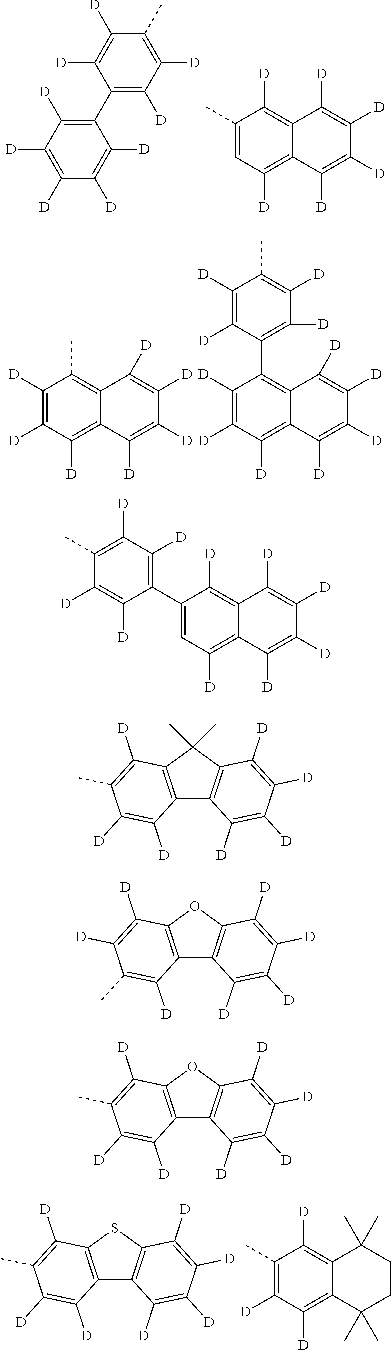

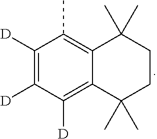

7. The compound of claim 1, wherein
the compound represented by Chemical Formula 1 is any one selected from the group consisting of:

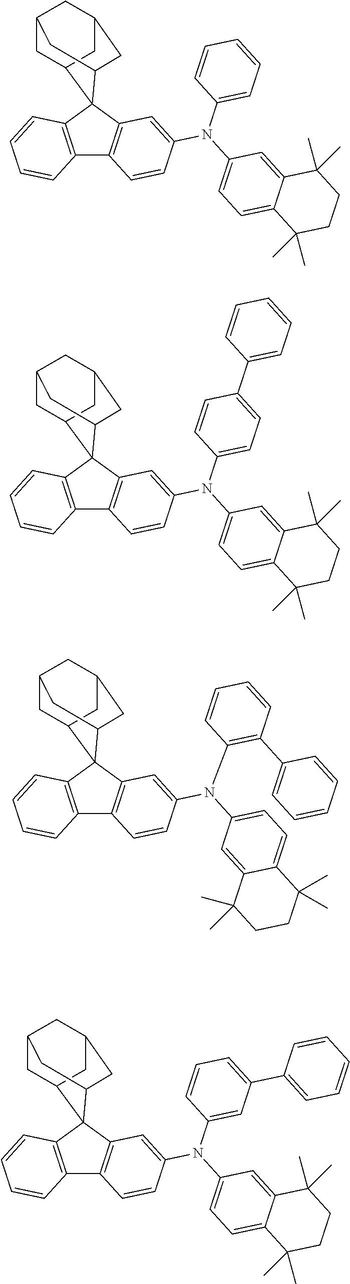

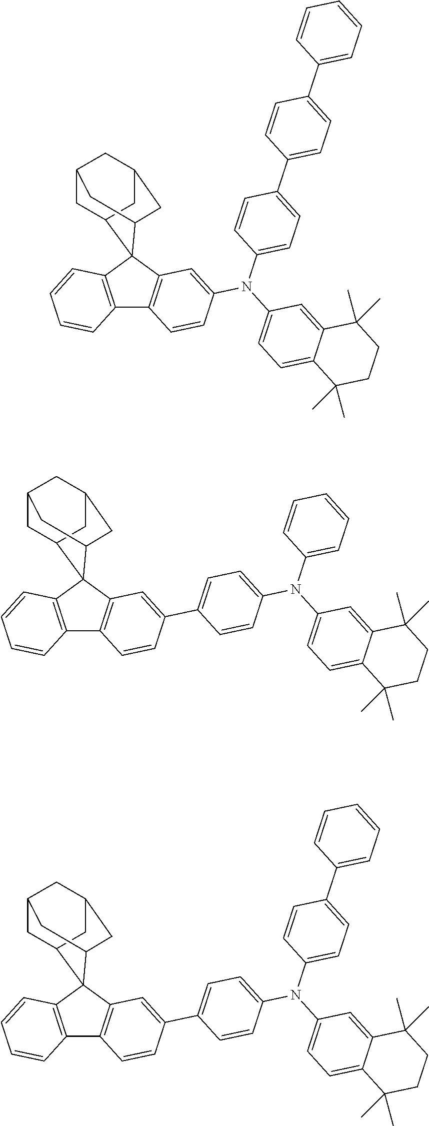 [SMILES: CC1(C)CCC(C)(C)c2cc(N(c3ccc(-c4ccc(-c5ccccc5)cc4)cc3)c3ccc4c(c3)C3(c5ccccc5-4)C4CC5CC(C4)CC3C5)ccc21 ; CC1(C)CCC(C)(C)c2cc(N(c3ccccc3)c3ccc(-c4ccc5c(c4)C4(c6ccccc6-5)C5CC6CC(C5)CC4C6)cc3)ccc21 ; CC1(C)CCC(C)(C)c2cc(N(c3ccc(-c4ccccc4)cc3)c3ccc(-c4ccc5c(c4)C4(c6ccccc6-5)C5CC6CC(C5)CC4C6)cc3)ccc21]

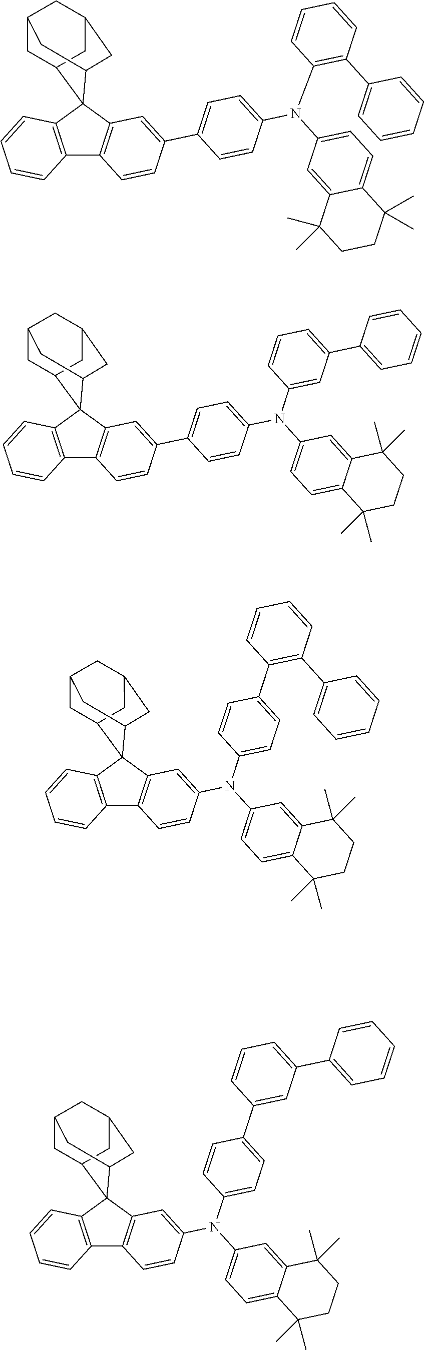 [SMILES: CC1(C)CCC(C)(C)c2cc(N(c3ccc(-c4ccc5c(c4)C4(c6ccccc6-5)C5CC6CC(C5)CC4C6)cc3)c3ccccc3-c3ccccc3)ccc21 ; CC1(C)CCC(C)(C)c2cc(N(c3ccc(-c4ccc5c(c4)C4(c6ccccc6-5)C5CC6CC(C5)CC4C6)cc3)c3cccc(-c4ccccc4)c3)ccc21 ; CC1(C)CCC(C)(C)c2cc(N(c3ccc(-c4ccccc4-c4ccccc4)cc3)c3ccc4c(c3)C3(c5ccccc5-4)C4CC5CC(C4)CC3C5)ccc21 ; CC1(C)CCC(C)(C)c2cc(N(c3ccc(-c4cccc(-c5ccccc5)c4)cc3)c3ccc4c(c3)C3(c5ccccc5-4)C4CC5CC(C4)CC3C5)ccc21]

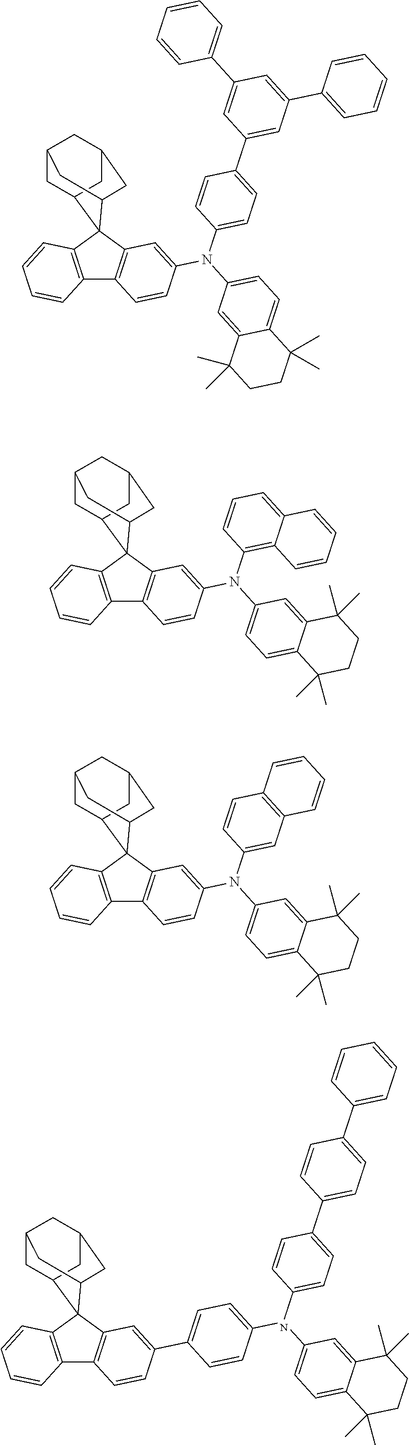 [SMILES: CC1(C)CCC(C)(C)c2cc(N(c3ccc(-c4cc(-c5ccccc5)cc(-c5ccccc5)c4)cc3)c3ccc4c(c3)C3(c5ccccc5-4)C4CC5CC(C4)CC3C5)ccc21 ; CC1(C)CCC(C)(C)c2cc(N(c3ccc4c(c3)C3(c5ccccc5-4)C4CC5CC(C4)CC3C5)c3cccc4ccccc34)ccc21 ; CC1(C)CCC(C)(C)c2cc(N(c3ccc4c(c3)C3(c5ccccc5-4)C4CC5CC(C4)CC3C5)c3ccc4ccccc4c3)ccc21 ; CC1(C)CCC(C)(C)c2cc(N(c3ccc(-c4ccc(-c5ccccc5)cc4)cc3)c3ccc(-c4ccc5c(c4)C4(c6ccccc6-5)C5CC6CC(C5)CC4C6)cc3)ccc21]

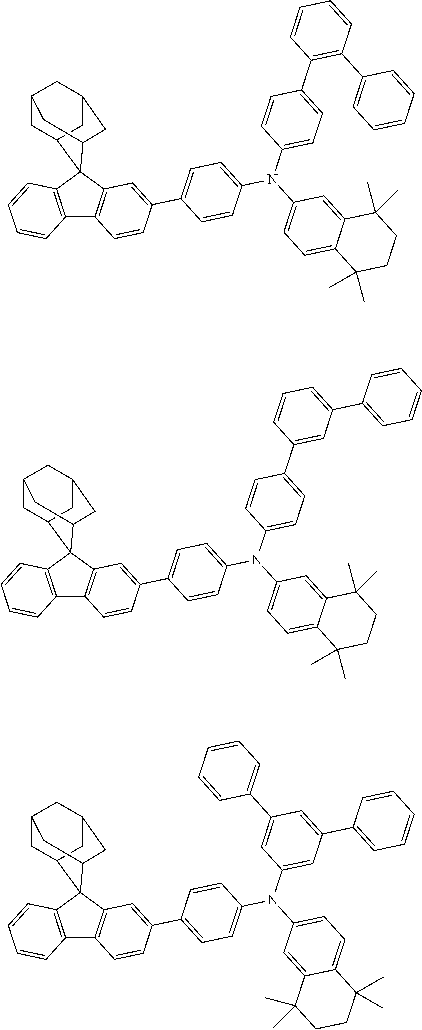 [SMILES: CC1(C)CCC(C)(C)c2cc(N(c3ccc(-c4ccc5c(c4)C4(c6ccccc6-5)C5CC6CC(C5)CC4C6)cc3)c3ccc(-c4ccccc4-c4ccccc4)cc3)ccc21 ; CC1(C)CCC(C)(C)c2cc(N(c3ccc(-c4cccc(-c5ccccc5)c4)cc3)c3ccc(-c4ccc5c(c4)C4(c6ccccc6-5)C5CC6CC(C5)CC4C6)cc3)ccc21 ; CC1(C)CCC(C)(C)c2cc(N(c3ccc(-c4ccc5c(c4)C4(c6ccccc6-5)C5CC6CC(C5)CC4C6)cc3)c3cc(-c4ccccc4)cc(-c4ccccc4)c3)ccc21]

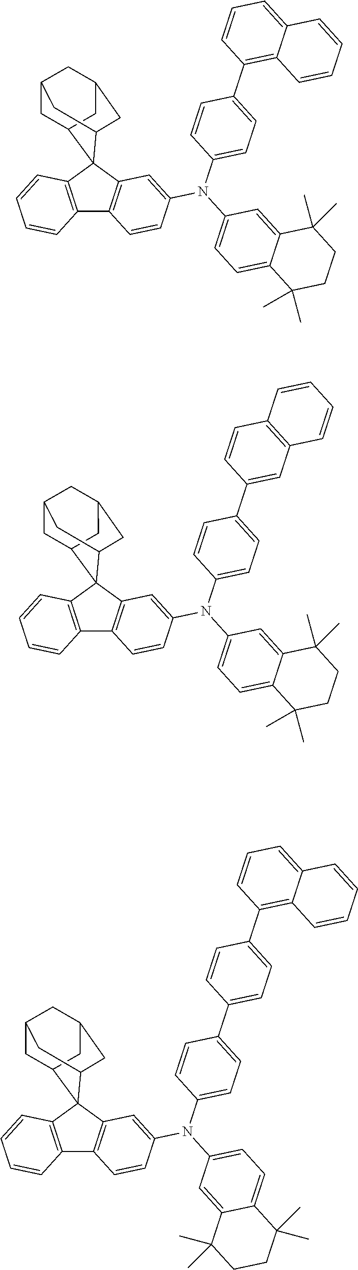 [SMILES: CC1(C)CCC(C)(C)c2cc(N(c3ccc(-c4cccc5ccccc45)cc3)c3ccc4c(c3)C3(c5ccccc5-4)C4CC5CC(C4)CC3C5)ccc21 ; CC1(C)CCC(C)(C)c2cc(N(c3ccc(-c4ccc5ccccc5c4)cc3)c3ccc4c(c3)C3(c5ccccc5-4)C4CC5CC(C4)CC3C5)ccc21 ; CC1(C)CCC(C)(C)c2cc(N(c3ccc(-c4ccc(-c5cccc6ccccc56)cc4)cc3)c3ccc4c(c3)C3(c5ccccc5-4)C4CC5CC(C4)CC3C5)ccc21]

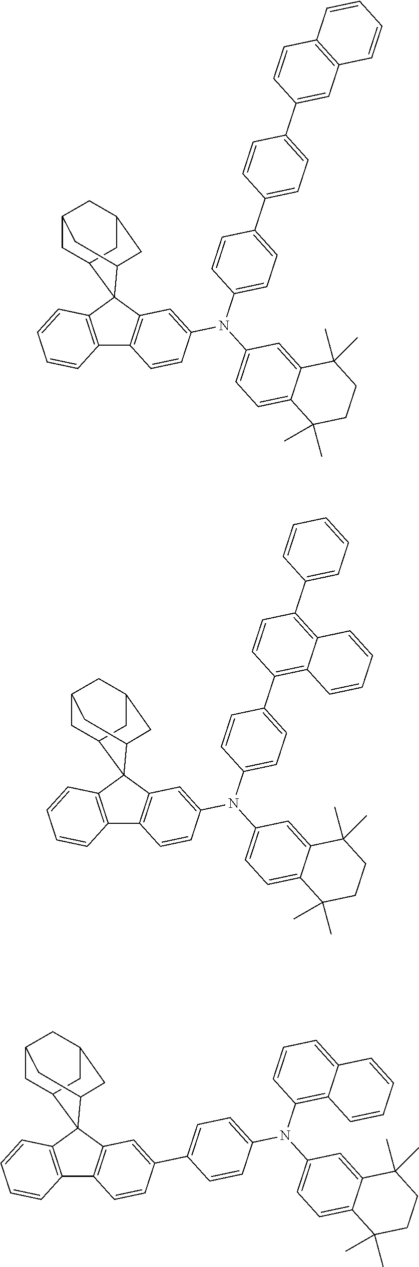 [SMILES: CC1(C)CCC(C)(C)c2cc(N(c3ccc(-c4ccc(-c5ccc6ccccc6c5)cc4)cc3)c3ccc4c(c3)C3(c5ccccc5-4)C4CC5CC(C4)CC3C5)ccc21 ; CC1(C)CCC(C)(C)c2cc(N(c3ccc(-c4ccc(-c5ccccc5)c5ccccc45)cc3)c3ccc4c(c3)C3(c5ccccc5-4)C4CC5CC(C4)CC3C5)ccc21 ; CC1(C)CCC(C)(C)c2cc(N(c3ccc(-c4ccc5c(c4)C4(c6ccccc6-5)C5CC6CC(C5)CC4C6)cc3)c3cccc4ccccc34)ccc21]

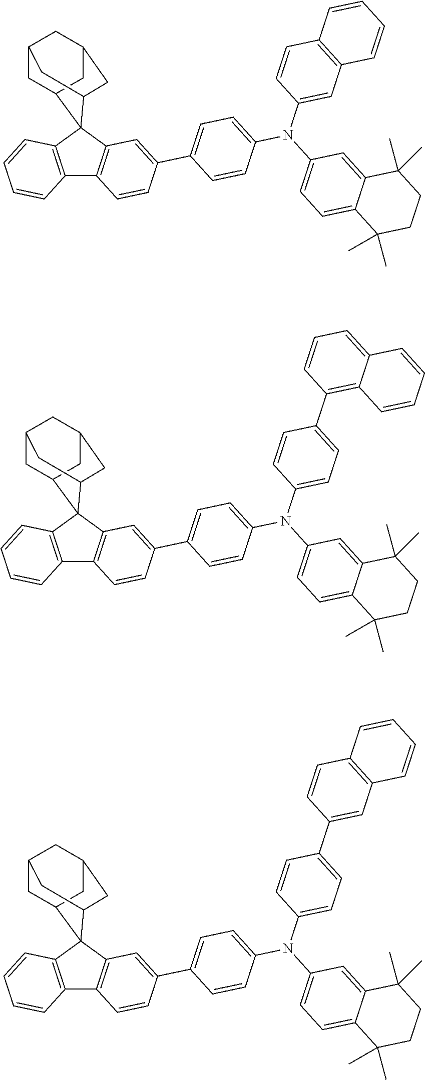 [SMILES: CC1(C)CCC(C)(C)c2cc(N(c3ccc(-c4ccc5c(c4)C4(c6ccccc6-5)C5CC6CC(C5)CC4C6)cc3)c3ccc4ccccc4c3)ccc21 ; CC1(C)CCC(C)(C)c2cc(N(c3ccc(-c4ccc5c(c4)C4(c6ccccc6-5)C5CC6CC(C5)CC4C6)cc3)c3ccc(-c4cccc5ccccc45)cc3)ccc21 ; CC1(C)CCC(C)(C)c2cc(N(c3ccc(-c4ccc5c(c4)C4(c6ccccc6-5)C5CC6CC(C5)CC4C6)cc3)c3ccc(-c4ccc5ccccc5c4)cc3)ccc21]

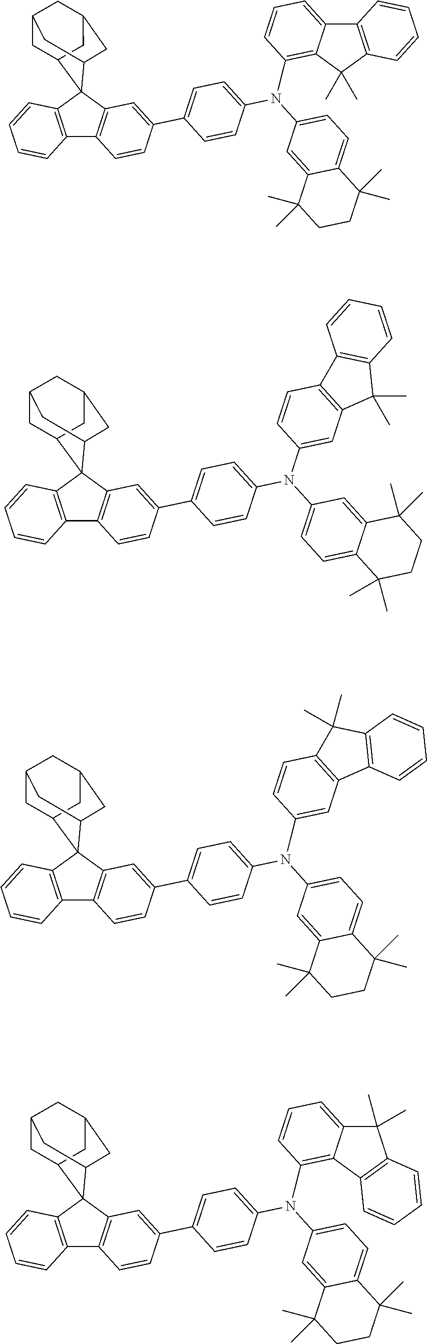 [SMILES: CC1(C)CCC(C)(C)c2cc(N(c3ccc(-c4ccc5c(c4)C4(c6ccccc6-5)C5CC6CC(C5)CC4C6)cc3)c3cccc4c3C(C)(C)c3ccccc3-4)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)-c3ccccc3C4(C)C)ccc21 ; CC1(C)CCC(C)(C)c2cc(N(c3ccc(-c4ccc5c(c4)C4(c6ccccc6-5)C5CC6CC(C5)CC4C6)cc3)c3cccc4c3-c3ccccc3C4(C)C)ccc21]

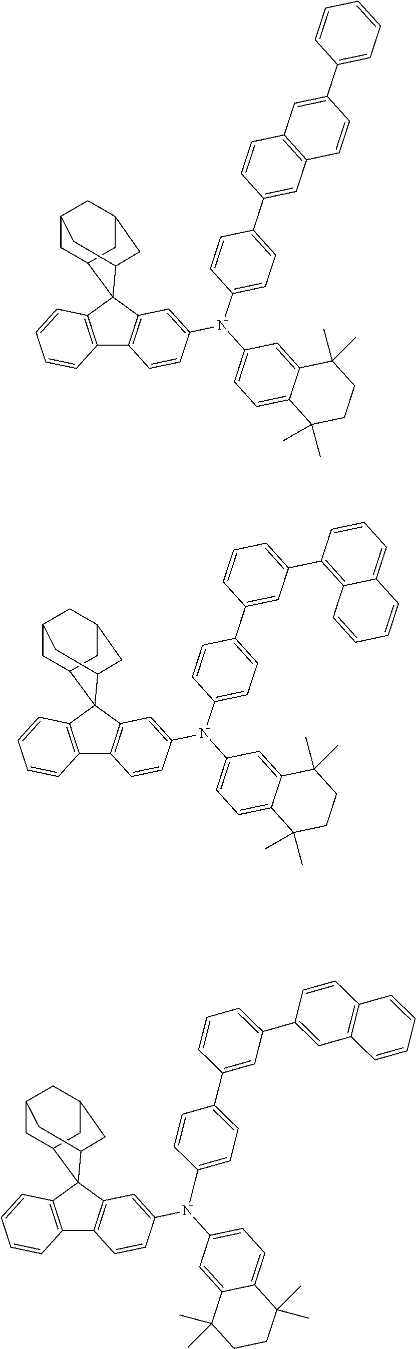 [SMILES: CC1(C)CCC(C)(C)c2cc(N(c3ccc(-c4ccc5cc(-c6ccccc6)ccc5c4)cc3)c3ccc4c(c3)C3(c5ccccc5-4)C4CC5CC(C4)CC3C5)ccc21 ; CC1(C)CCC(C)(C)c2cc(N(c3ccc(-c4cccc(-c5cccc6ccccc56)c4)cc3)c3ccc4c(c3)C3(c5ccccc5-4)C4CC5CC(C4)CC3C5)ccc21 ; CC1(C)CCC(C)(C)c2cc(N(c3ccc(-c4cccc(-c5ccc6ccccc6c5)c4)cc3)c3ccc4c(c3)C3(c5ccccc5-4)C4CC5CC(C4)CC3C5)ccc21]

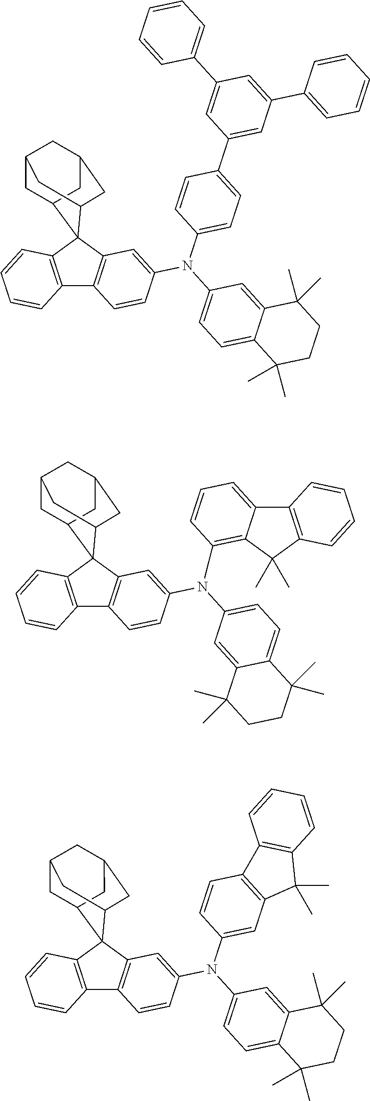 [SMILES: CC1(C)CCC(C)(C)c2cc(N(c3ccc(-c4cc(-c5ccccc5)cc(-c5ccccc5)c4)cc3)c3ccc4c(c3)C3(c5ccccc5-4)C4CC5CC(C4)CC3C5)ccc21 ; CC1(C)CCC(C)(C)c2cc(N(c3ccc4c(c3)C3(c5ccccc5-4)C4CC5CC(C4)CC3C5)c3cccc4c3C(C)(C)c3ccccc3-4)ccc21 ; CC1(C)CCC(C)(C)c2cc(N(c3ccc4c(c3)C(C)(C)c3ccccc3-4)c3ccc4c(c3)C3(c5ccccc5-4)C4CC5CC(C4)CC3C5)ccc21]

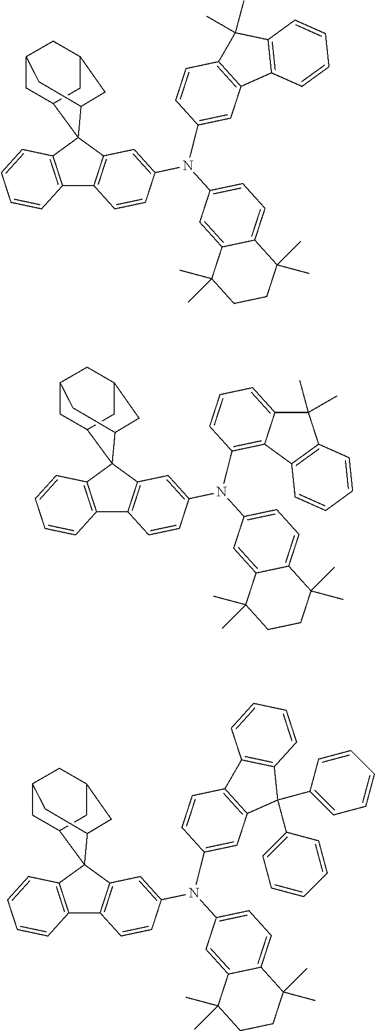 [SMILES: CC1(C)CCC(C)(C)c2cc(N(c3ccc4c(c3)-c3ccccc3C4(C)C)c3ccc4c(c3)C3(c5ccccc5-4)C4CC5CC(C4)CC3C5)ccc21 ; CC1(C)CCC(C)(C)c2cc(N(c3ccc4c(c3)C3(c5ccccc5-4)C4CC5CC(C4)CC3C5)c3cccc4c3-c3ccccc3C4(C)C)ccc21 ; CC1(C)CCC(C)(C)c2cc(N(c3ccc4c(c3)C(c3ccccc3)(c3ccccc3)c3ccccc3-4)c3ccc4c(c3)C3(c5ccccc5-4)C4CC5CC(C4)CC3C5)ccc21]

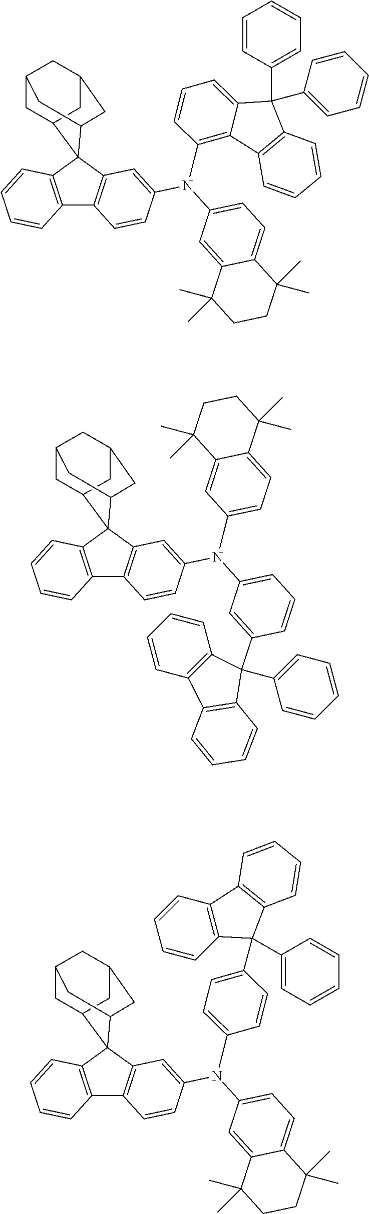 [SMILES: CC1(C)CCC(C)(C)c2cc(N(c3ccc4c(c3)C3(c5ccccc5-4)C4CC5CC(C4)CC3C5)c3cccc4c3-c3ccccc3C4(c3ccccc3)c3ccccc3)ccc21 ; CC1(C)CCC(C)(C)c2cc(N(c3cccc(C4(c5ccccc5)c5ccccc5-c5ccccc54)c3)c3ccc4c(c3)C3(c5ccccc5-4)C4CC5CC(C4)CC3C5)ccc21 ; CC1(C)CCC(C)(C)c2cc(N(c3ccc(C4(c5ccccc5)c5ccccc5-c5ccccc54)cc3)c3ccc4c(c3)C3(c5ccccc5-4)C4CC5CC(C4)CC3C5)ccc21]

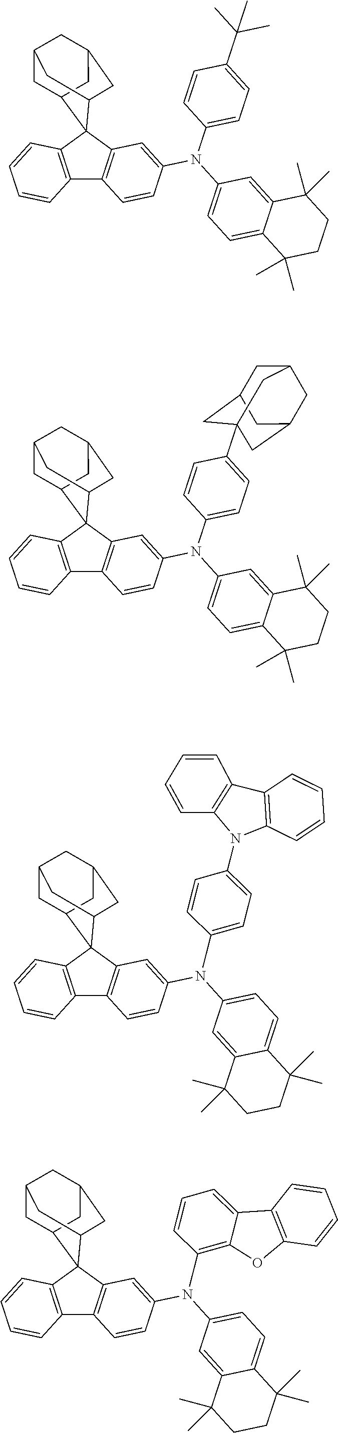 [SMILES: CC(C)(C)c1ccc(N(c2ccc3c(c2)C(C)(C)CCC3(C)C)c2ccc3c(c2)C2(c4ccccc4-3)C3CC4CC(C3)CC2C4)cc1 ; CC1(C)CCC(C)(C)c2cc(N(c3ccc(C45CC6CC(CC(C6)C4)C5)cc3)c3ccc4c(c3)C3(c5ccccc5-4)C4CC5CC(C4)CC3C5)ccc21 ; CC1(C)CCC(C)(C)c2cc(N(c3ccc(-n4c5ccccc5c5ccccc54)cc3)c3ccc4c(c3)C3(c5ccccc5-4)C4CC5CC(C4)CC3C5)ccc21 ; CC1(C)CCC(C)(C)c2cc(N(c3ccc4c(c3)C3(c5ccccc5-4)C4CC5CC(C4)CC3C5)c3cccc4c3oc3ccccc34)ccc21]

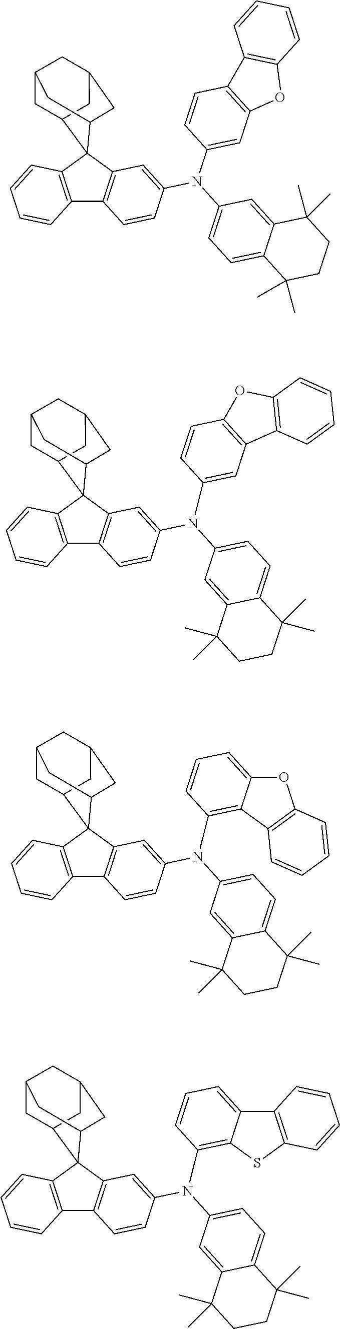 [SMILES: CC1(C)CCC(C)(C)c2cc(N(c3ccc4c(c3)C3(c5ccccc5-4)C4CC5CC(C4)CC3C5)c3ccc4c(c3)oc3ccccc34)ccc21 ; CC1(C)CCC(C)(C)c2cc(N(c3ccc4c(c3)C3(c5ccccc5-4)C4CC5CC(C4)CC3C5)c3ccc4oc5ccccc5c4c3)ccc21 ; CC1(C)CCC(C)(C)c2cc(N(c3ccc4c(c3)C3(c5ccccc5-4)C4CC5CC(C4)CC3C5)c3cccc4oc5ccccc5c34)ccc21 ; CC1(C)CCC(C)(C)c2cc(N(c3ccc4c(c3)C3(c5ccccc5-4)C4CC5CC(C4)CC3C5)c3cccc4c3sc3ccccc34)ccc21]

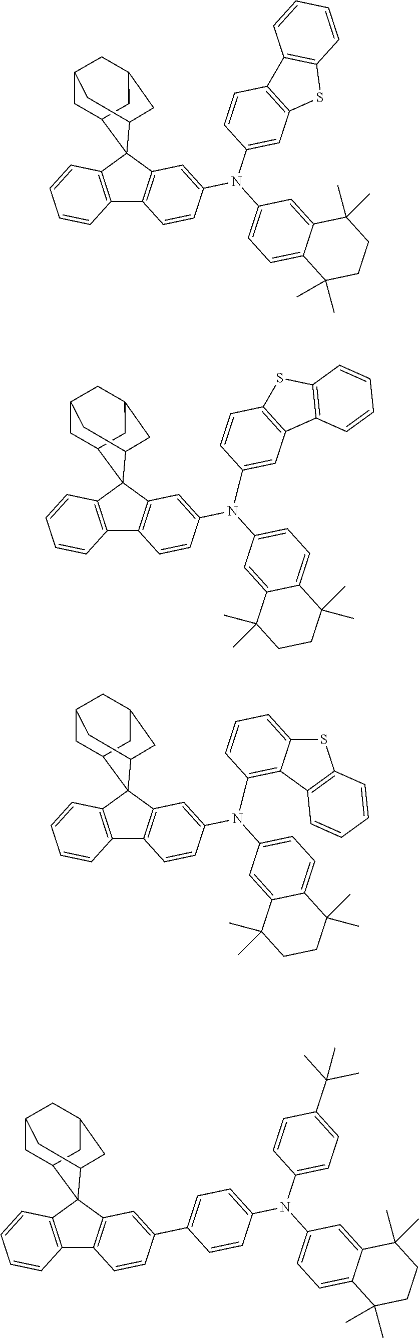 [SMILES: CC1(C)CCC(C)(C)c2cc(N(c3ccc4c(c3)C3(c5ccccc5-4)C4CC5CC(C4)CC3C5)c3ccc4c(c3)sc3ccccc34)ccc21 ; CC1(C)CCC(C)(C)c2cc(N(c3ccc4c(c3)C3(c5ccccc5-4)C4CC5CC(C4)CC3C5)c3ccc4sc5ccccc5c4c3)ccc21 ; CC1(C)CCC(C)(C)c2cc(N(c3ccc4c(c3)C3(c5ccccc5-4)C4CC5CC(C4)CC3C5)c3cccc4sc5ccccc5c34)ccc21 ; CC(C)(C)c1ccc(N(c2ccc(-c3ccc4c(c3)C3(c5ccccc5-4)C4CC5CC(C4)CC3C5)cc2)c2ccc3c(c2)C(C)(C)CCC3(C)C)cc1]

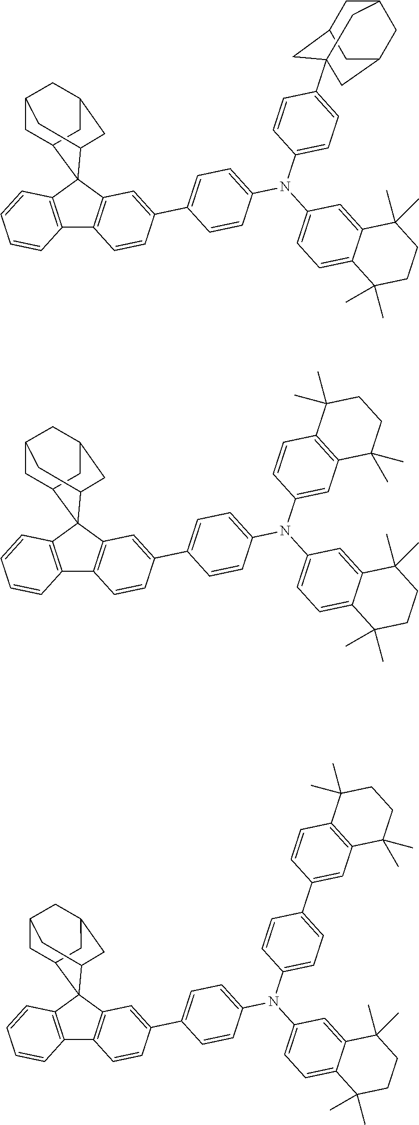 [SMILES: CC1(C)CCC(C)(C)c2cc(N(c3ccc(-c4ccc5c(c4)C4(c6ccccc6-5)C5CC6CC(C5)CC4C6)cc3)c3ccc(C45CC6CC(CC(C6)C4)C5)cc3)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)C(C)(C)CCC4(C)C)ccc21 ; CC1(C)CCC(C)(C)c2cc(-c3ccc(N(c4ccc(-c5ccc6c(c5)C5(c7ccccc7-6)C6CC7CC(C6)CC5C7)cc4)c4ccc5c(c4)C(C)(C)CCC5(C)C)cc3)ccc21]

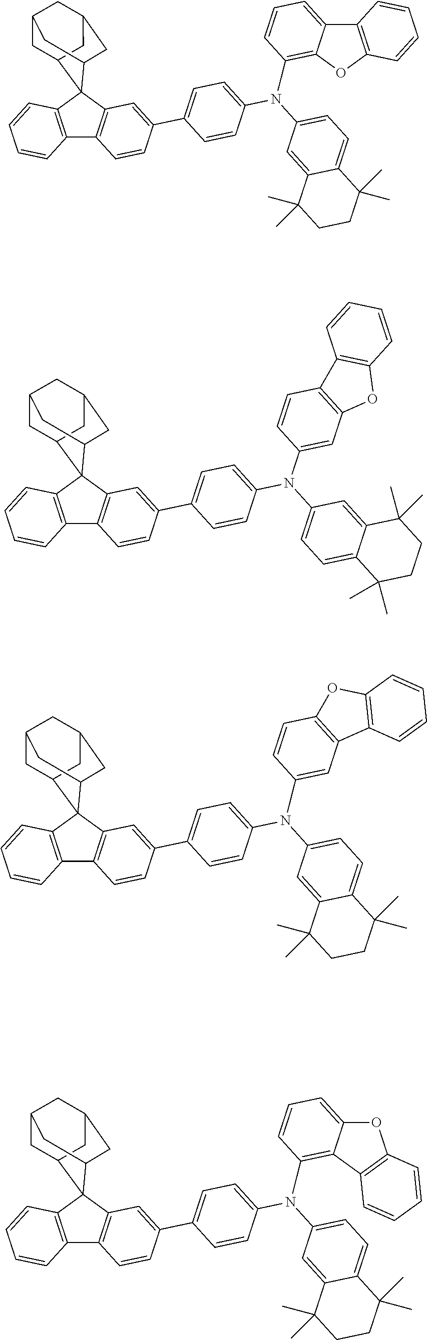 [SMILES: CC1(C)CCC(C)(C)c2cc(N(c3ccc(-c4ccc5c(c4)C4(c6ccccc6-5)C5CC6CC(C5)CC4C6)cc3)c3cccc4c3oc3ccccc34)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)oc3ccccc34)ccc21 ; CC1(C)CCC(C)(C)c2cc(N(c3ccc(-c4ccc5c(c4)C4(c6ccccc6-5)C5CC6CC(C5)CC4C6)cc3)c3ccc4oc5ccccc5c4c3)ccc21 ; CC1(C)CCC(C)(C)c2cc(N(c3ccc(-c4ccc5c(c4)C4(c6ccccc6-5)C5CC6CC(C5)CC4C6)cc3)c3cccc4oc5ccccc5c34)ccc21]

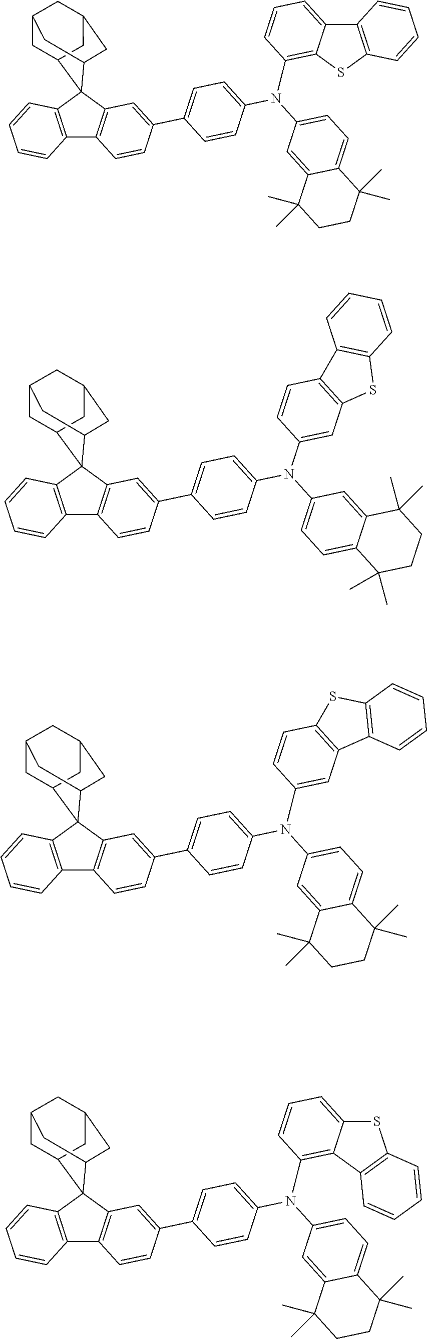 [SMILES: CC1(C)CCC(C)(C)c2cc(N(c3ccc(-c4ccc5c(c4)C4(c6ccccc6-5)C5CC6CC(C5)CC4C6)cc3)c3cccc4c3sc3ccccc34)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)sc3ccccc34)ccc21 ; CC1(C)CCC(C)(C)c2cc(N(c3ccc(-c4ccc5c(c4)C4(c6ccccc6-5)C5CC6CC(C5)CC4C6)cc3)c3ccc4sc5ccccc5c4c3)ccc21 ; CC1(C)CCC(C)(C)c2cc(N(c3ccc(-c4ccc5c(c4)C4(c6ccccc6-5)C5CC6CC(C5)CC4C6)cc3)c3cccc4sc5ccccc5c34)ccc21]

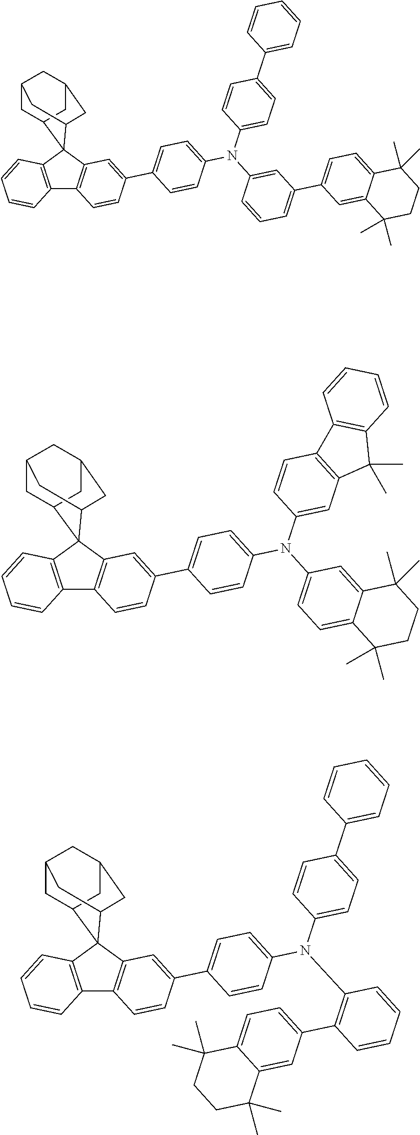 [SMILES: CC1(C)CCC(C)(C)c2cc(-c3cccc(N(c4ccc(-c5ccccc5)cc4)c4ccc(-c5ccc6c(c5)C5(c7ccccc7-6)C6CC7CC(C6)CC5C7)cc4)c3)ccc21 ; CC1(C)CCC(C)(C)c2cc(N(c3ccc(-c4ccc5c(c4)C4(c6ccccc6-5)C5CC6CC(C5)CC4C6)cc3)c3ccc4c(c3)C(C)(C)c3ccccc3-4)ccc21 ; CC1(C)CCC(C)(C)c2cc(-c3ccccc3N(c3ccc(-c4ccccc4)cc3)c3ccc(-c4ccc5c(c4)C4(c6ccccc6-5)C5CC6CC(C5)CC4C6)cc3)ccc21]

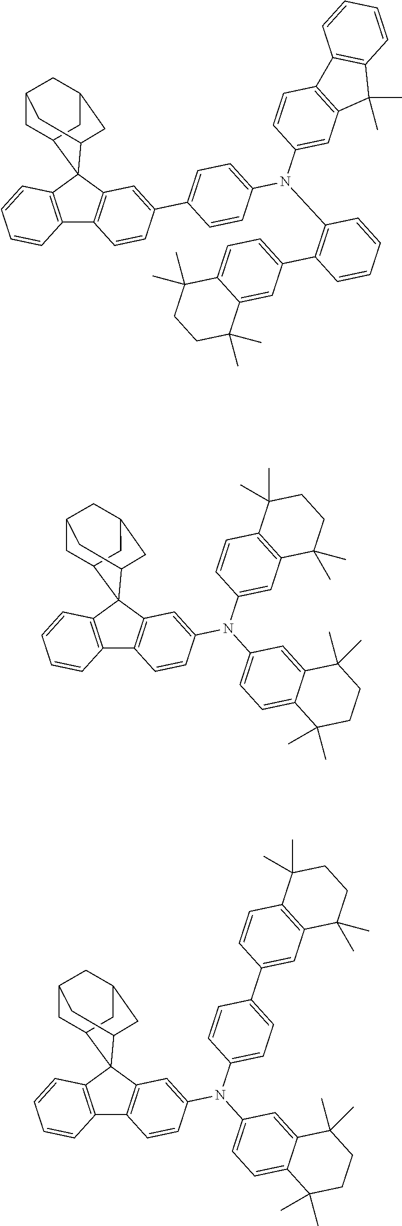 [SMILES: CC1(C)CCC(C)(C)c2cc(-c3ccccc3N(c3ccc(-c4ccc5c(c4)C4(c6ccccc6-5)C5CC6CC(C5)CC4C6)cc3)c3ccc4c(c3)C(C)(C)c3ccccc3-4)ccc21 ; CC1(C)CCC(C)(C)c2cc(N(c3ccc4c(c3)C(C)(C)CCC4(C)C)c3ccc4c(c3)C3(c5ccccc5-4)C4CC5CC(C4)CC3C5)ccc21 ; CC1(C)CCC(C)(C)c2cc(-c3ccc(N(c4ccc5c(c4)C(C)(C)CCC5(C)C)c4ccc5c(c4)C4(c6ccccc6-5)C5CC6CC(C5)CC4C6)cc3)ccc21]

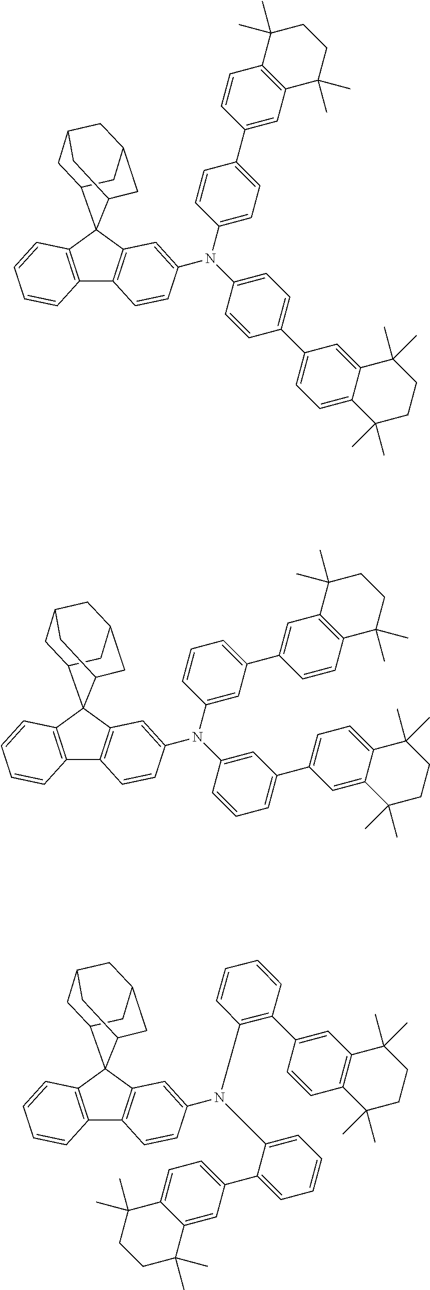 [SMILES: CC1(C)CCC(C)(C)c2cc(-c3ccc(N(c4ccc(-c5ccc6c(c5)C(C)(C)CCC6(C)C)cc4)c4ccc5c(c4)C4(c6ccccc6-5)C5CC6CC(C5)CC4C6)cc3)ccc21 ; CC1(C)CCC(C)(C)c2cc(-c3cccc(N(c4cccc(-c5ccc6c(c5)C(C)(C)CCC6(C)C)c4)c4ccc5c(c4)C4(c6ccccc6-5)C5CC6CC(C5)CC4C6)c3)ccc21 ; CC1(C)CCC(C)(C)c2cc(-c3ccccc3N(c3ccc4c(c3)C3(c5ccccc5-4)C4CC5CC(C4)CC3C5)c3ccccc3-c3ccc4c(c3)C(C)(C)CCC4(C)C)ccc21]

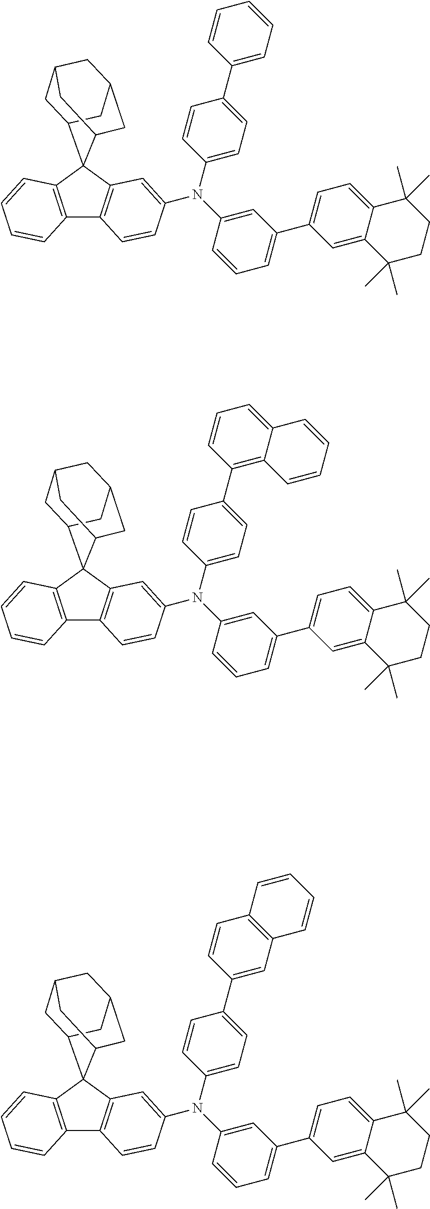 [SMILES: CC1(C)CCC(C)(C)c2cc(-c3cccc(N(c4ccc(-c5ccccc5)cc4)c4ccc5c(c4)C4(c6ccccc6-5)C5CC6CC(C5)CC4C6)c3)ccc21 ; CC1(C)CCC(C)(C)c2cc(-c3cccc(N(c4ccc(-c5cccc6ccccc56)cc4)c4ccc5c(c4)C4(c6ccccc6-5)C5CC6CC(C5)CC4C6)c3)ccc21 ; CC1(C)CCC(C)(C)c2cc(-c3cccc(N(c4ccc(-c5ccc6ccccc6c5)cc4)c4ccc5c(c4)C4(c6ccccc6-5)C5CC6CC(C5)CC4C6)c3)ccc21]

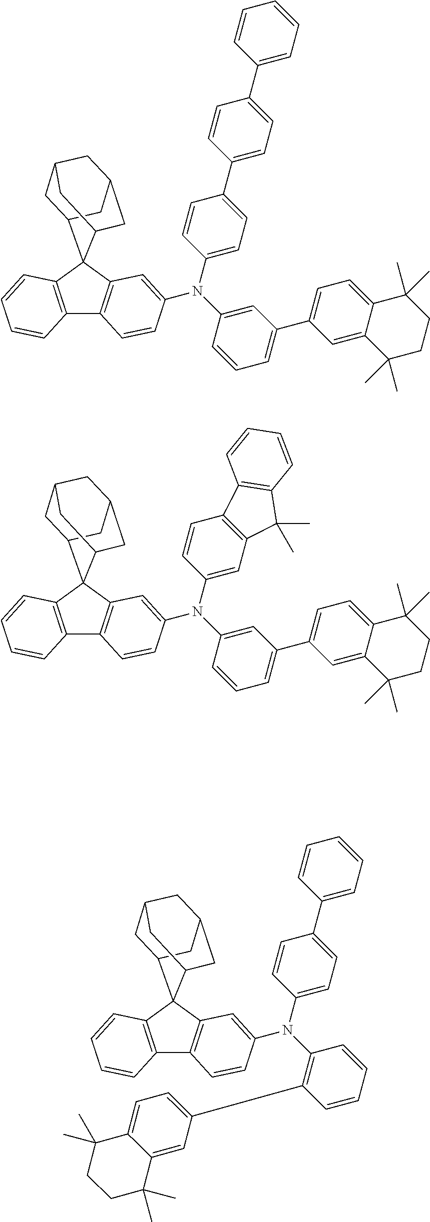

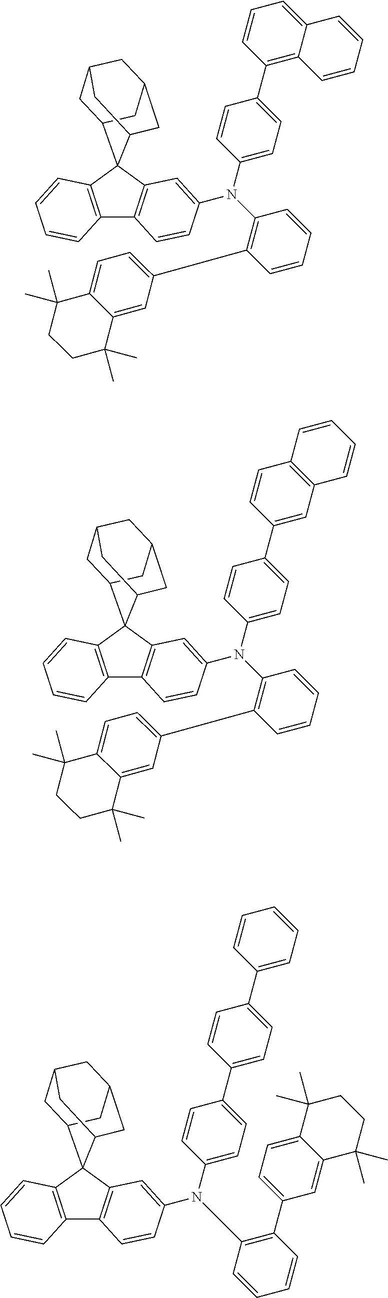 [SMILES: CC1(C)CCC(C)(C)c2cc(-c3ccccc3N(c3ccc(-c4cccc5ccccc45)cc3)c3ccc4c(c3)C3(c5ccccc5-4)C4CC5CC(C4)CC3C5)ccc21 ; CC1(C)CCC(C)(C)c2cc(-c3ccccc3N(c3ccc(-c4ccc5ccccc5c4)cc3)c3ccc4c(c3)C3(c5ccccc5-4)C4CC5CC(C4)CC3C5)ccc21 ; CC1(C)CCC(C)(C)c2cc(-c3ccccc3N(c3ccc(-c4ccc(-c5ccccc5)cc4)cc3)c3ccc4c(c3)C3(c5ccccc5-4)C4CC5CC(C4)CC3C5)ccc21]

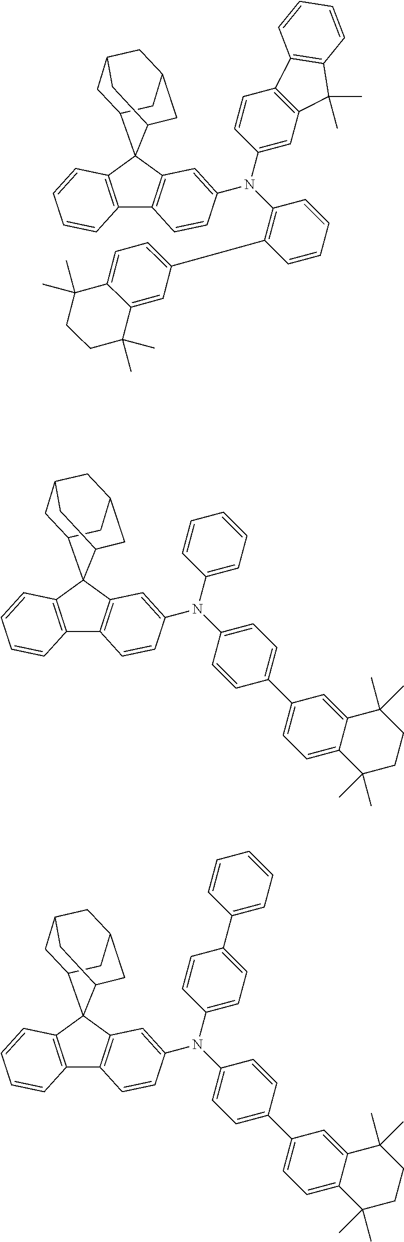

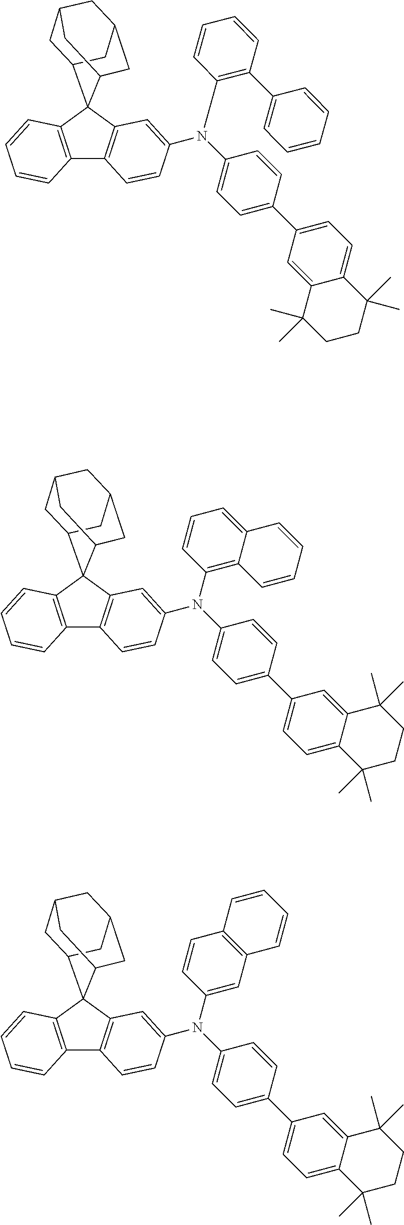 [SMILES: CC1(C)CCC(C)(C)c2cc(-c3ccc(N(c4ccc5c(c4)C4(c6ccccc6-5)C5CC6CC(C5)CC4C6)c4ccccc4-c4ccccc4)cc3)ccc21 ; CC1(C)CCC(C)(C)c2cc(-c3ccc(N(c4ccc5c(c4)C4(c6ccccc6-5)C5CC6CC(C5)CC4C6)c4cccc5ccccc45)cc3)ccc21 ; CC1(C)CCC(C)(C)c2cc(-c3ccc(N(c4ccc5c(c4)C4(c6ccccc6-5)C5CC6CC(C5)CC4C6)c4ccc5ccccc5c4)cc3)ccc21]

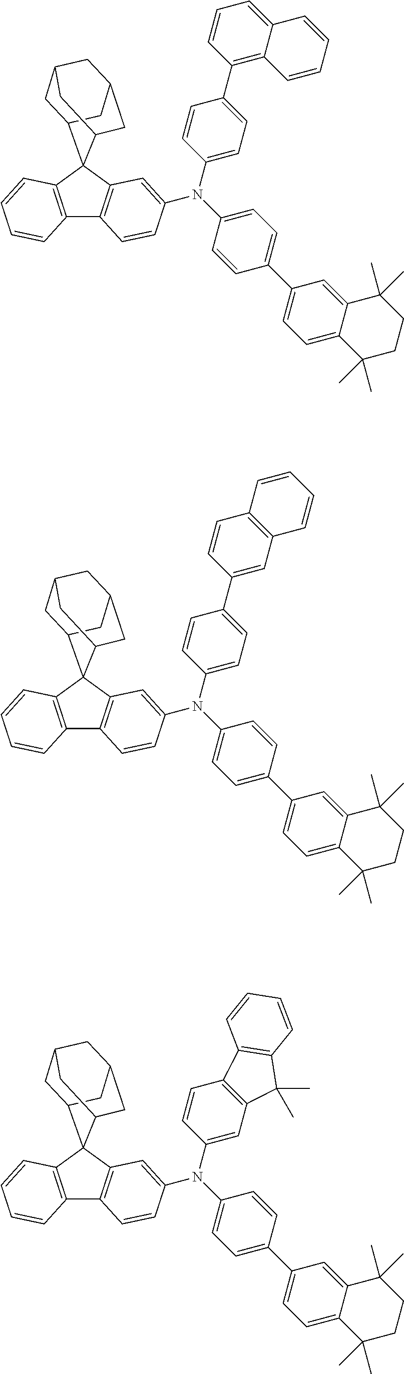 [SMILES: CC1(C)CCC(C)(C)c2cc(-c3ccc(N(c4ccc(-c5cccc6ccccc56)cc4)c4ccc5c(c4)C4(c6ccccc6-5)C5CC6CC(C5)CC4C6)cc3)ccc21 ; CC1(C)CCC(C)(C)c2cc(-c3ccc(N(c4ccc(-c5ccc6ccccc6c5)cc4)c4ccc5c(c4)C4(c6ccccc6-5)C5CC6CC(C5)CC4C6)cc3)ccc21 ; CC1(C)CCC(C)(C)c2cc(-c3ccc(N(c4ccc5c(c4)C(C)(C)c4ccccc4-5)c4ccc5c(c4)C4(c6ccccc6-5)C5CC6CC(C5)CC4C6)cc3)ccc21]

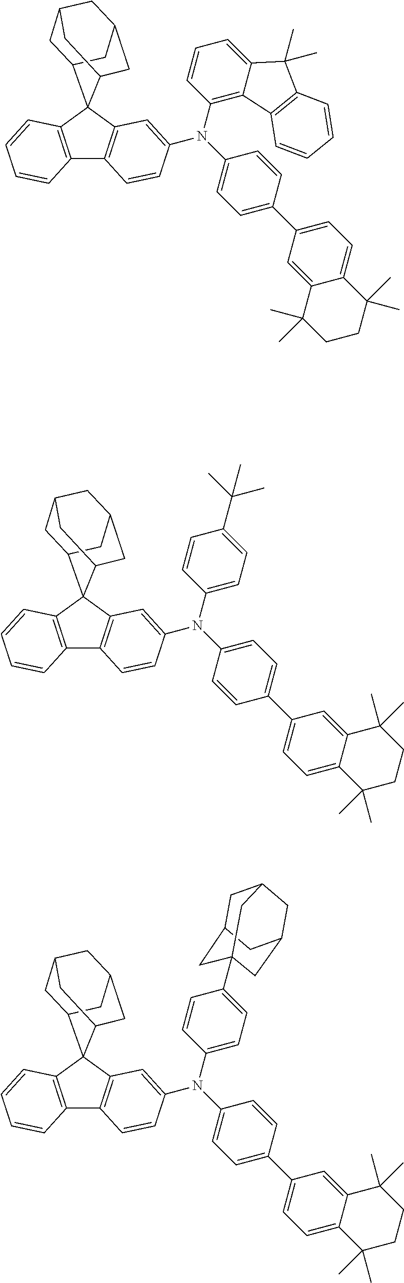 [SMILES: CC1(C)CCC(C)(C)c2cc(-c3ccc(N(c4ccc5c(c4)C4(c6ccccc6-5)C5CC6CC(C5)CC4C6)c4cccc5c4-c4ccccc4C5(C)C)cc3)ccc21 ; CC(C)(C)c1ccc(N(c2ccc(-c3ccc4c(c3)C(C)(C)CCC4(C)C)cc2)c2ccc3c(c2)C2(c4ccccc4-3)C3CC4CC(C3)CC2C4)cc1 ; CC1(C)CCC(C)(C)c2cc(-c3ccc(N(c4ccc(C56CC7CC(CC(C7)C5)C6)cc4)c4ccc5c(c4)C4(c6ccccc6-5)C5CC6CC(C5)CC4C6)cc3)ccc21]

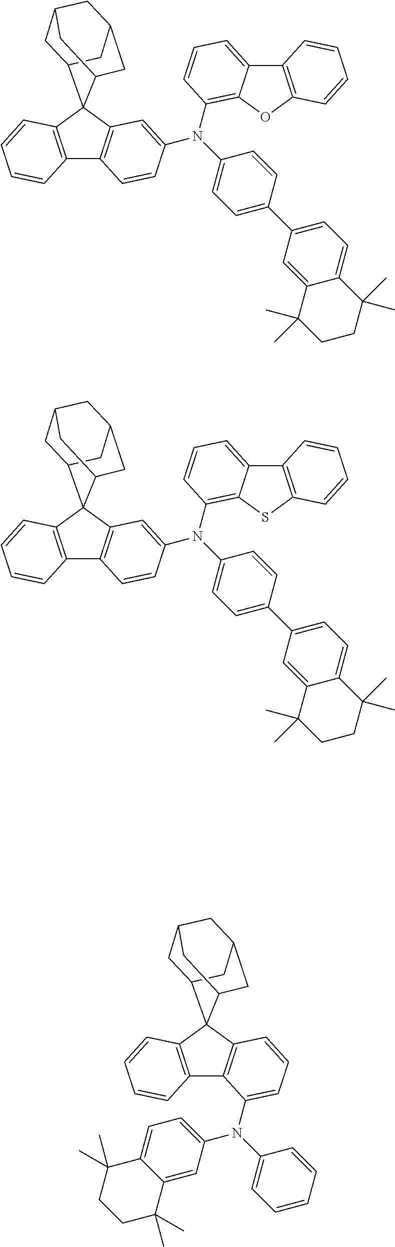

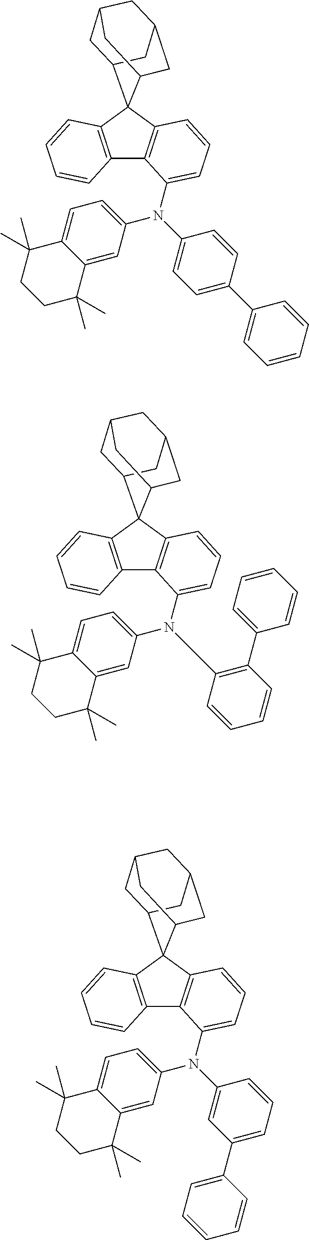 [SMILES: CC1(C)CCC(C)(C)c2cc(N(c3ccc(-c4ccccc4)cc3)c3cccc4c3-c3ccccc3C43C4CC5CC(C4)CC3C5)ccc21 ; CC1(C)CCC(C)(C)c2cc(N(c3ccccc3-c3ccccc3)c3cccc4c3-c3ccccc3C43C4CC5CC(C4)CC3C5)ccc21 ; CC1(C)CCC(C)(C)c2cc(N(c3cccc(-c4ccccc4)c3)c3cccc4c3-c3ccccc3C43C4CC5CC(C4)CC3C5)ccc21]

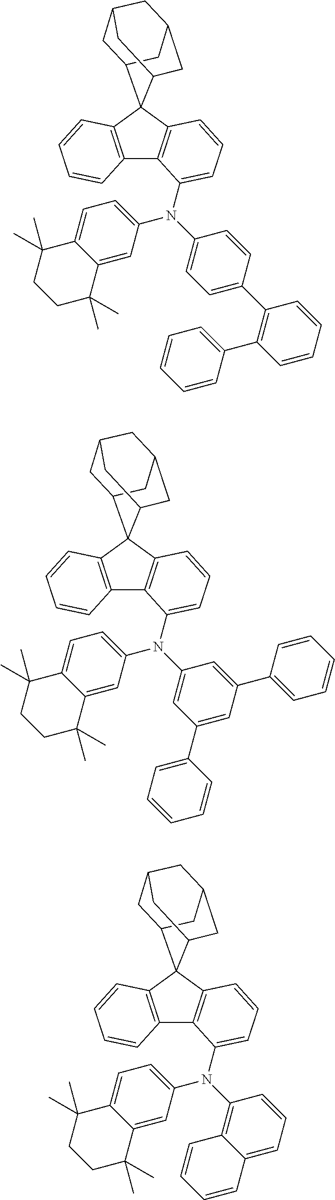

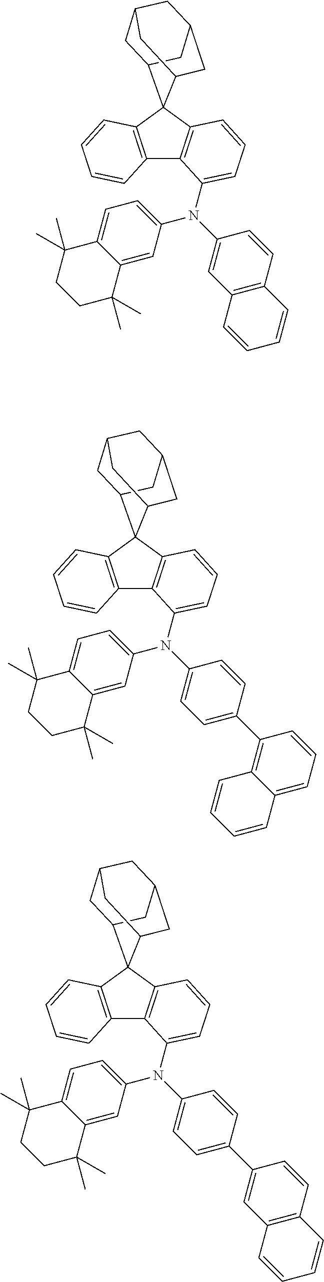 [SMILES: CC1(C)CCC(C)(C)c2cc(N(c3ccc4ccccc4c3)c3cccc4c3-c3ccccc3C43C4CC5CC(C4)CC3C5)ccc21 ; CC1(C)CCC(C)(C)c2cc(N(c3ccc(-c4cccc5ccccc45)cc3)c3cccc4c3-c3ccccc3C43C4CC5CC(C4)CC3C5)ccc21 ; CC1(C)CCC(C)(C)c2cc(N(c3ccc(-c4ccc5ccccc5c4)cc3)c3cccc4c3-c3ccccc3C43C4CC5CC(C4)CC3C5)ccc21]

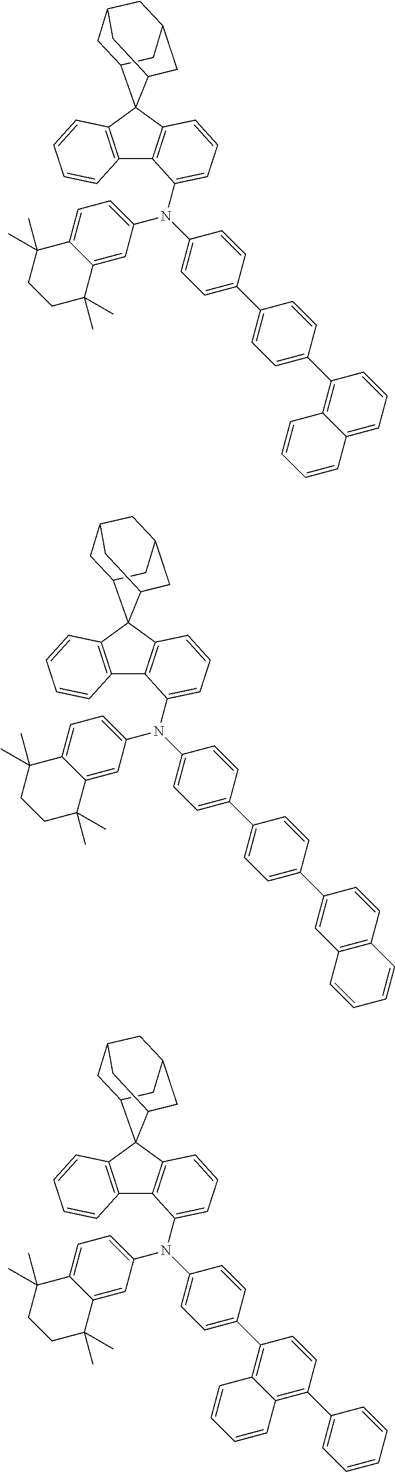 [SMILES: CC1(C)CCC(C)(C)c2cc(N(c3ccc(-c4ccc(-c5cccc6ccccc56)cc4)cc3)c3cccc4c3-c3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3cccc4c3-c3ccccc3C43C4CC5CC(C4)CC3C5)ccc21 ; CC1(C)CCC(C)(C)c2cc(N(c3ccc(-c4ccc(-c5ccccc5)c5ccccc45)cc3)c3cccc4c3-c3ccccc3C43C4CC5CC(C4)CC3C5)ccc21]

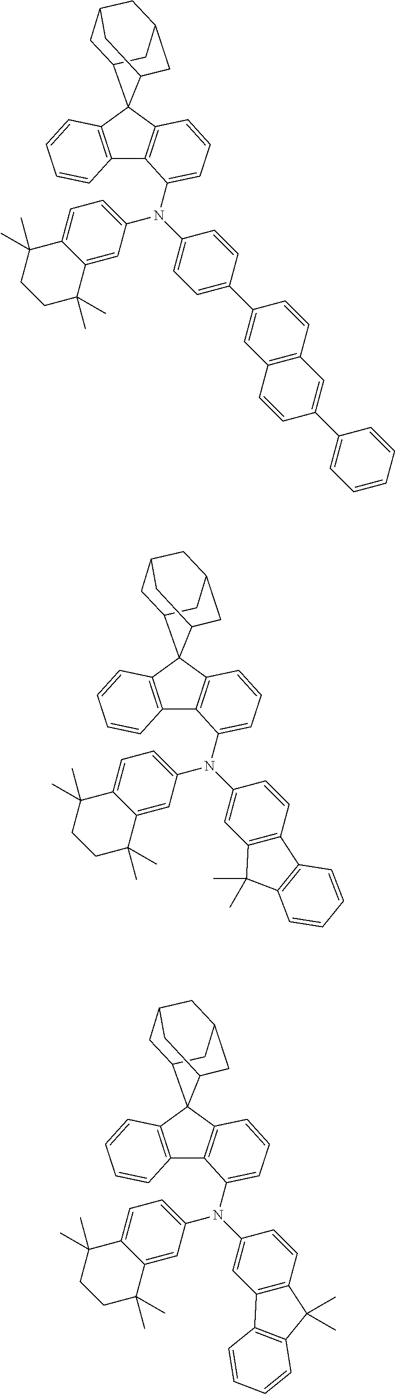

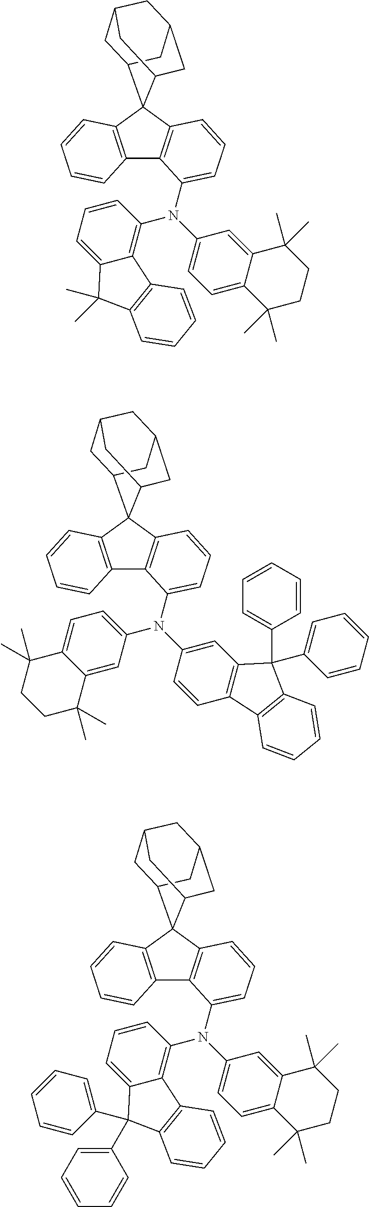

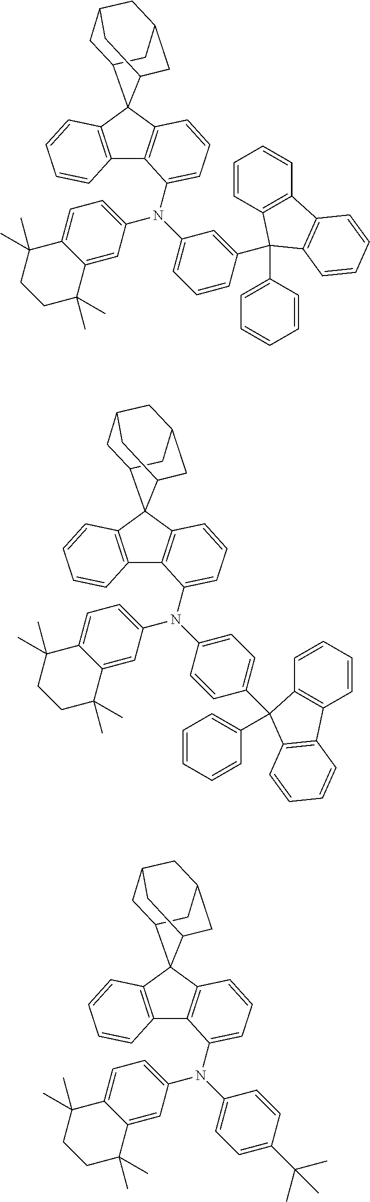 [SMILES: CC1(C)CCC(C)(C)c2cc(N(c3cccc(C4(c5ccccc5)c5ccccc5-c5ccccc54)c3)c3cccc4c3-c3ccccc3C43C4CC5CC(C4)CC3C5)ccc21 ; CC1(C)CCC(C)(C)c2cc(N(c3ccc(C4(c5ccccc5)c5ccccc5-c5ccccc54)cc3)c3cccc4c3-c3ccccc3C43C4CC5CC(C4)CC3C5)ccc21 ; CC(C)(C)c1ccc(N(c2ccc3c(c2)C(C)(C)CCC3(C)C)c2cccc3c2-c2ccccc2C32C3CC4CC(C3)CC2C4)cc1]

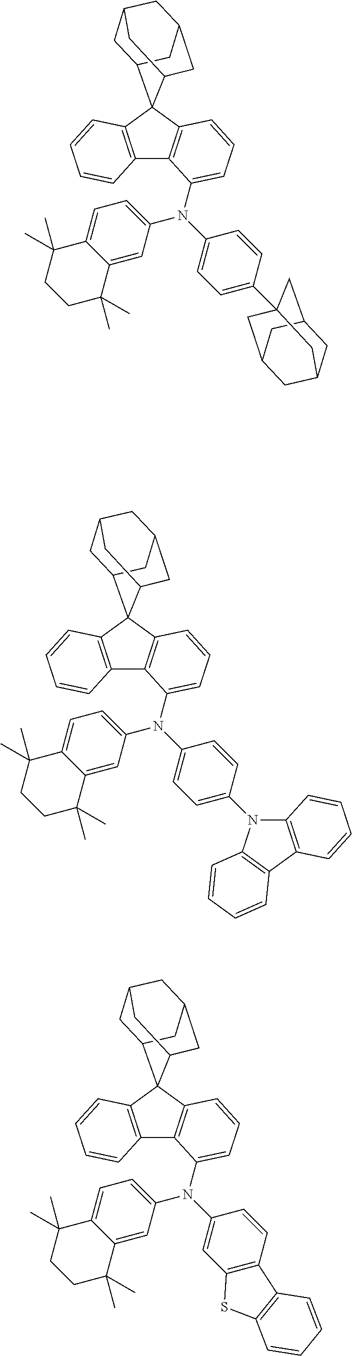 [SMILES: CC1(C)CCC(C)(C)c2cc(N(c3ccc(C45CC6CC(CC(C6)C4)C5)cc3)c3cccc4c3-c3ccccc3C43C4CC5CC(C4)CC3C5)ccc21 ; CC1(C)CCC(C)(C)c2cc(N(c3ccc(-n4c5ccccc5c5ccccc54)cc3)c3cccc4c3-c3ccccc3C43C4CC5CC(C4)CC3C5)ccc21 ; CC1(C)CCC(C)(C)c2cc(N(c3ccc4c(c3)sc3ccccc34)c3cccc4c3-c3ccccc3C43C4CC5CC(C4)CC3C5)ccc21]

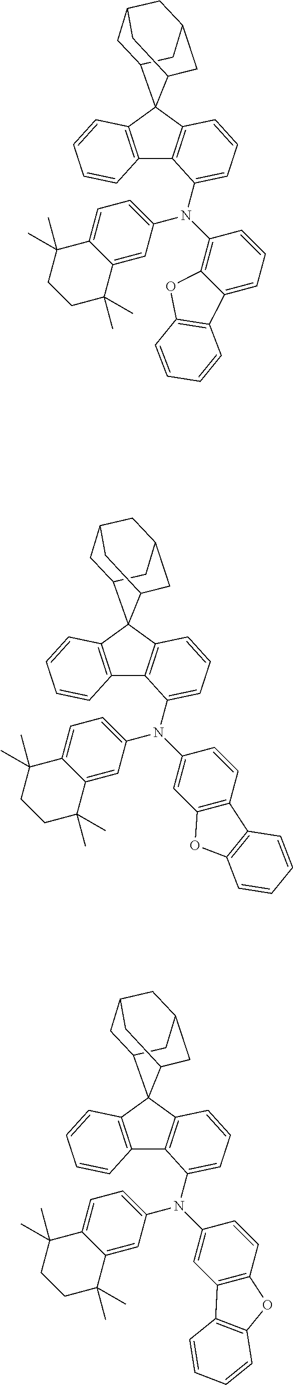 [SMILES: CC1(C)CCC(C)(C)c2cc(N(c3cccc4c3-c3ccccc3C43C4CC5CC(C4)CC3C5)c3cccc4c3oc3ccccc34)ccc21 ; CC1(C)CCC(C)(C)c2cc(N(c3ccc4c(c3)oc3ccccc34)c3cccc4c3-c3ccccc3C43C4CC5CC(C4)CC3C5)ccc21 ; CC1(C)CCC(C)(C)c2cc(N(c3ccc4oc5ccccc5c4c3)c3cccc4c3-c3ccccc3C43C4CC5CC(C4)CC3C5)ccc21]

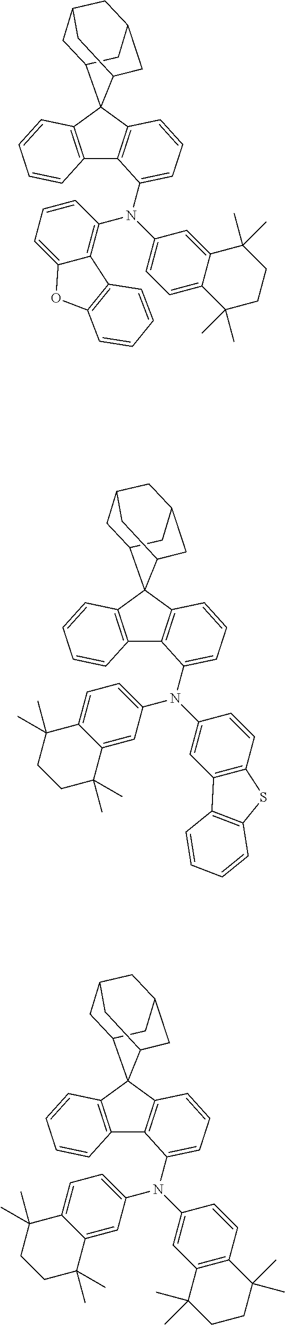

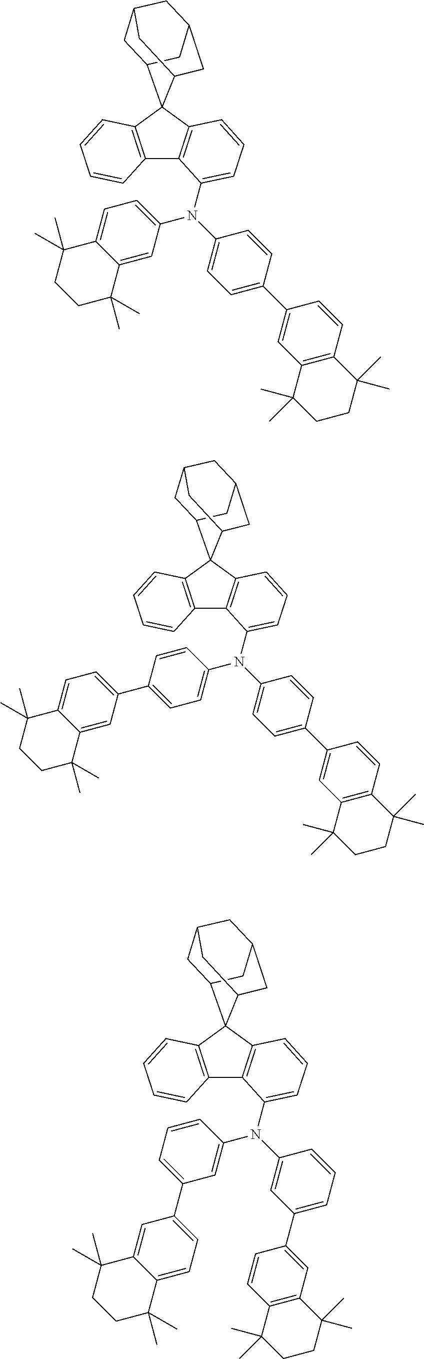

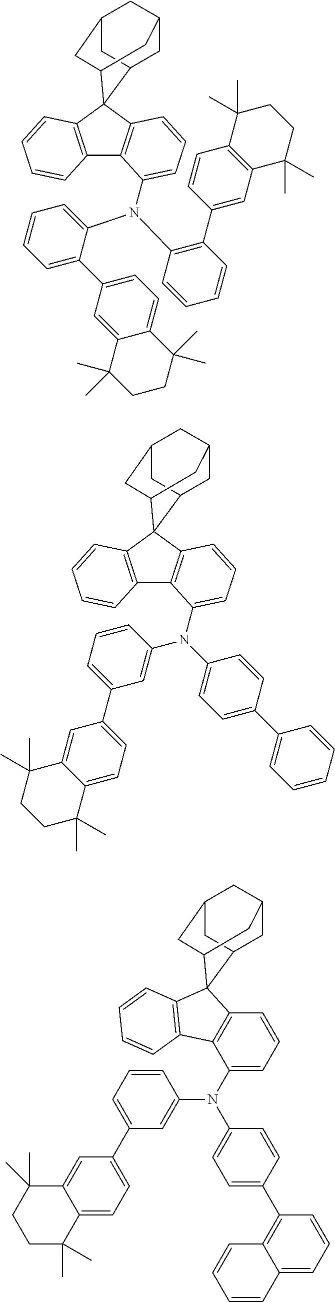

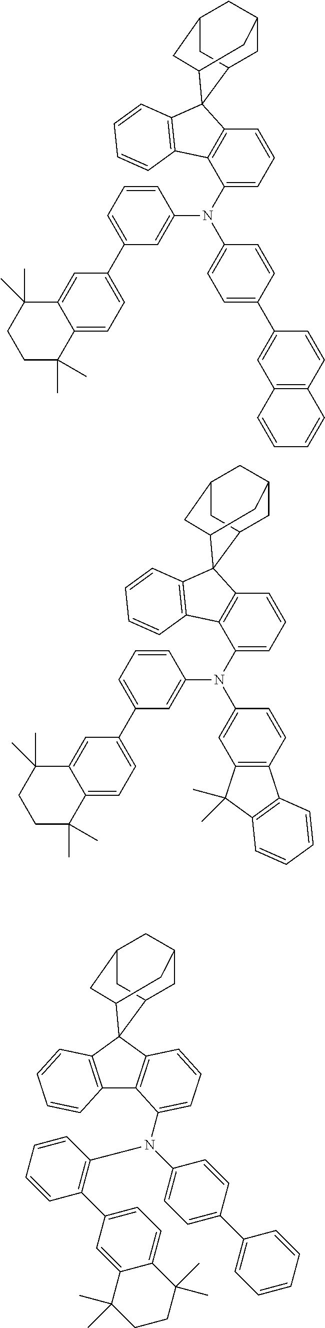

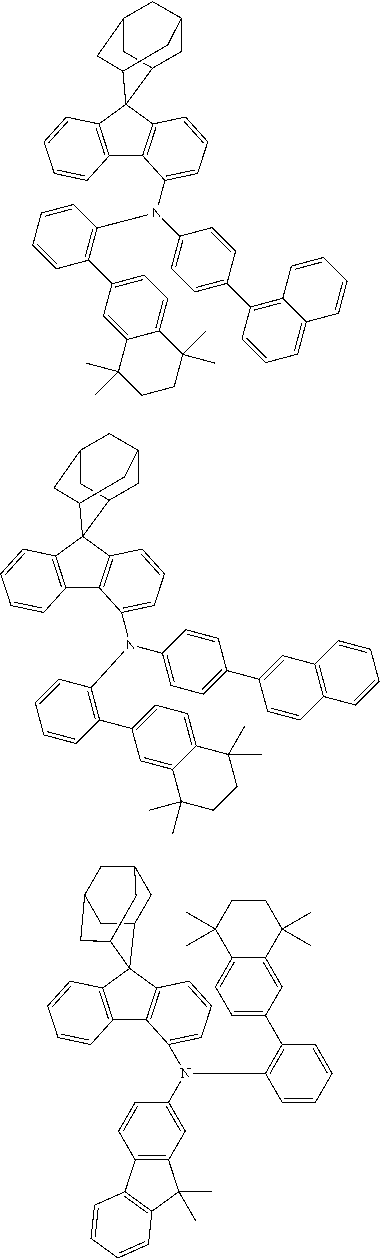

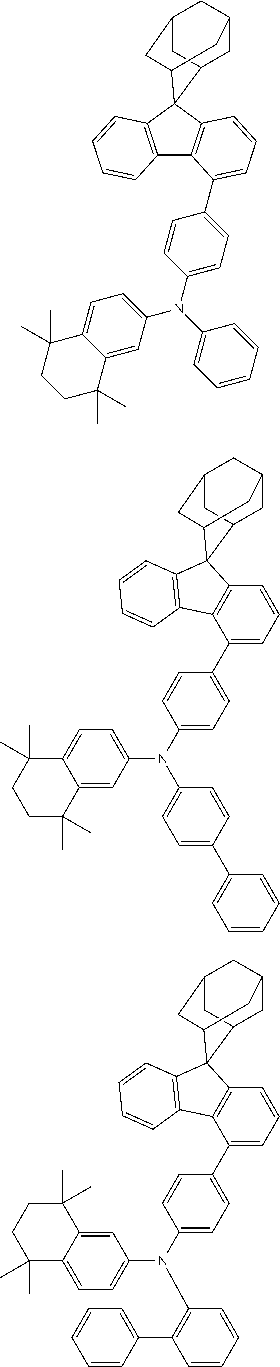

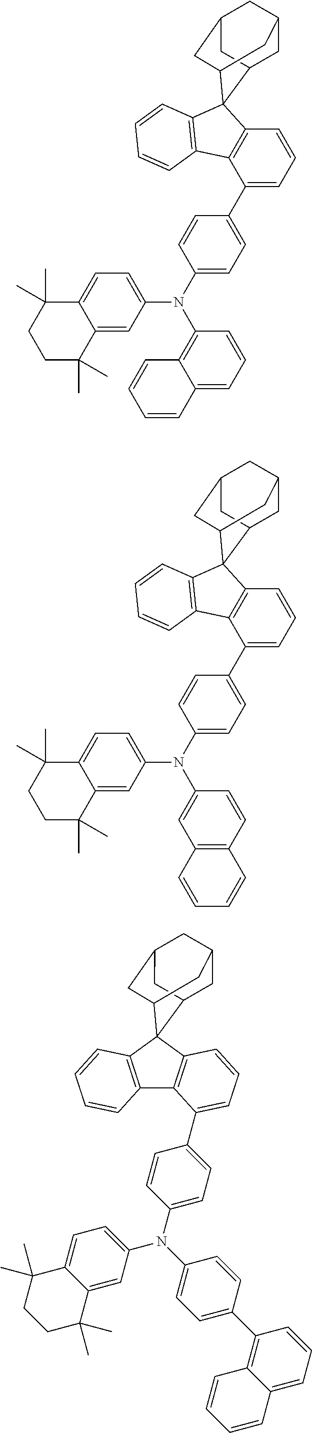

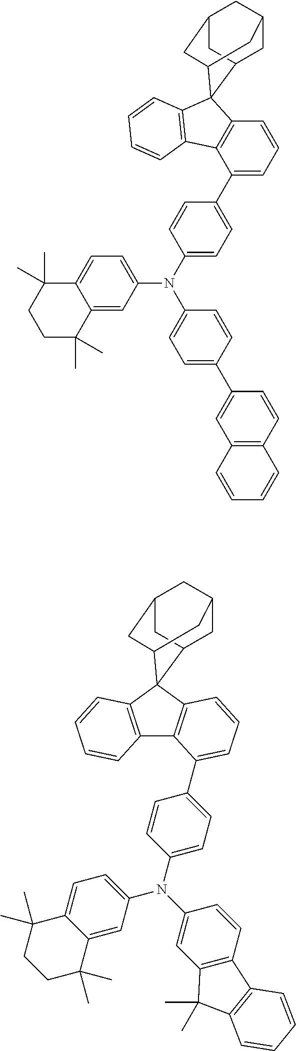 [SMILES: CC1(C)CCC(C)(C)c2cc(N(c3ccc(-c4ccc5ccccc5c4)cc3)c3ccc(-c4cccc5c4-c4ccccc4C54C5CC6CC(C5)CC4C6)cc3)ccc21 ; CC1(C)CCC(C)(C)c2cc(N(c3ccc(-c4cccc5c4-c4ccccc4C54C5CC6CC(C5)CC4C6)cc3)c3ccc4c(c3)C(C)(C)c3ccccc3-4)ccc21]

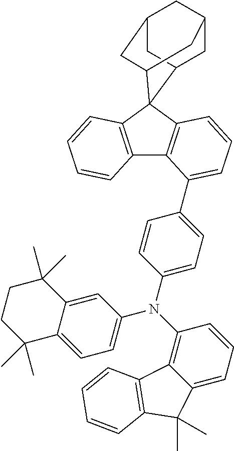 [SMILES: CC1(C)CCC(C)(C)c2cc(N(c3ccc(-c4cccc5c4-c4ccccc4C54C5CC6CC(C5)CC4C6)cc3)c3cccc4c3-c3ccccc3C4(C)C)ccc21]

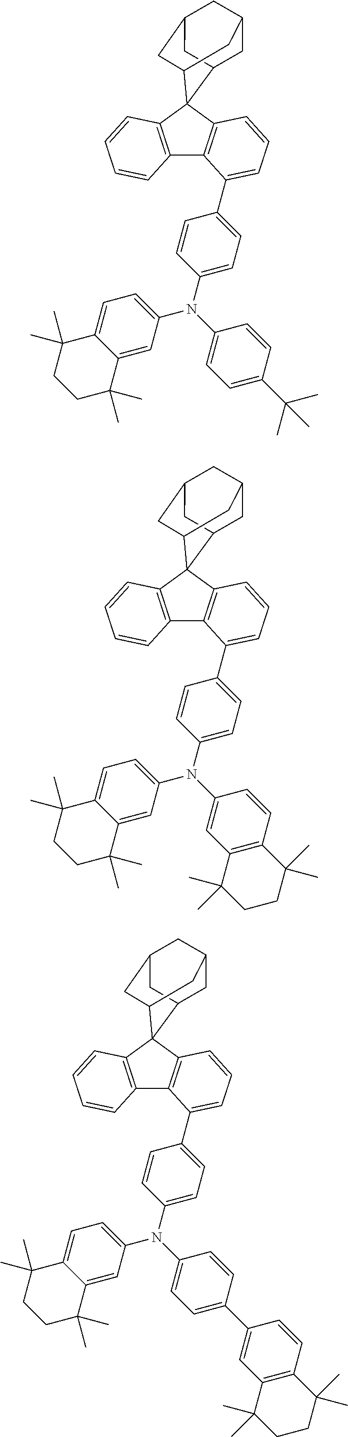

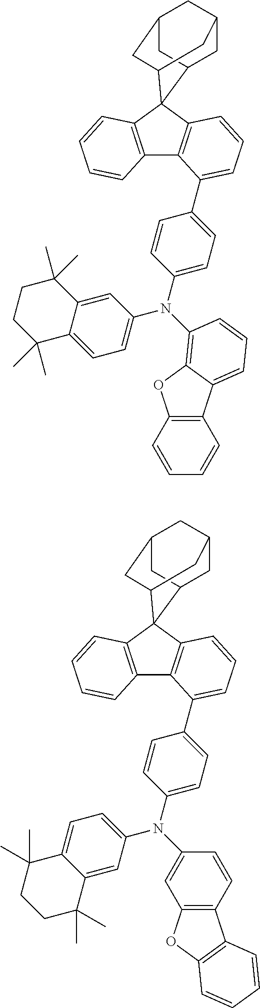 [SMILES: CC1(C)CCC(C)(C)c2cc(N(c3ccc(-c4cccc5c4-c4ccccc4C54C5CC6CC(C5)CC4C6)cc3)c3cccc4c3oc3ccccc34)ccc21 ; CC1(C)CCC(C)(C)c2cc(N(c3ccc(-c4cccc5c4-c4ccccc4C54C5CC6CC(C5)CC4C6)cc3)c3ccc4c(c3)oc3ccccc34)ccc21]

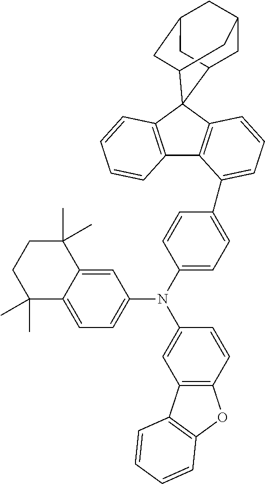 [SMILES: CC1(C)CCC(C)(C)c2cc(N(c3ccc(-c4cccc5c4-c4ccccc4C54C5CC6CC(C5)CC4C6)cc3)c3ccc4oc5ccccc5c4c3)ccc21]

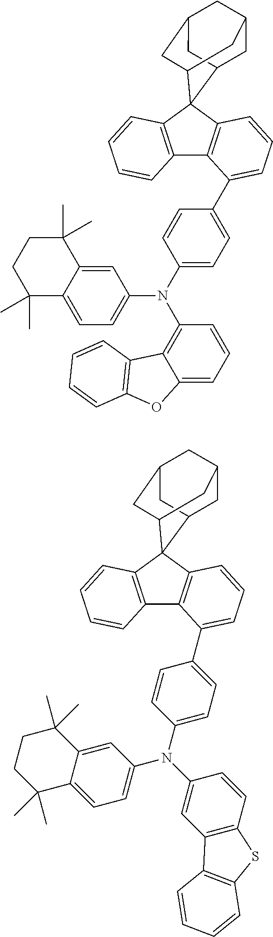 [SMILES: CC1(C)CCC(C)(C)c2cc(N(c3ccc(-c4cccc5c4-c4ccccc4C54C5CC6CC(C5)CC4C6)cc3)c3cccc4oc5ccccc5c34)ccc21 ; CC1(C)CCC(C)(C)c2cc(N(c3ccc(-c4cccc5c4-c4ccccc4C54C5CC6CC(C5)CC4C6)cc3)c3ccc4sc5ccccc5c4c3)ccc21]

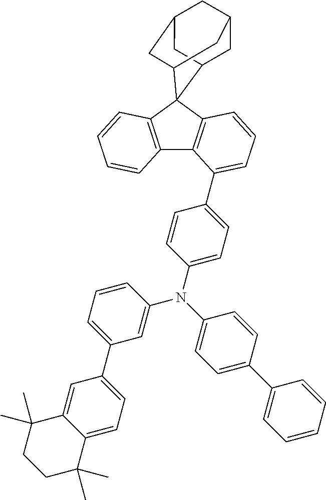 [SMILES: CC1(C)CCC(C)(C)c2cc(-c3cccc(N(c4ccc(-c5ccccc5)cc4)c4ccc(-c5cccc6c5-c5ccccc5C65C6CC7CC(C6)CC5C7)cc4)c3)ccc21]

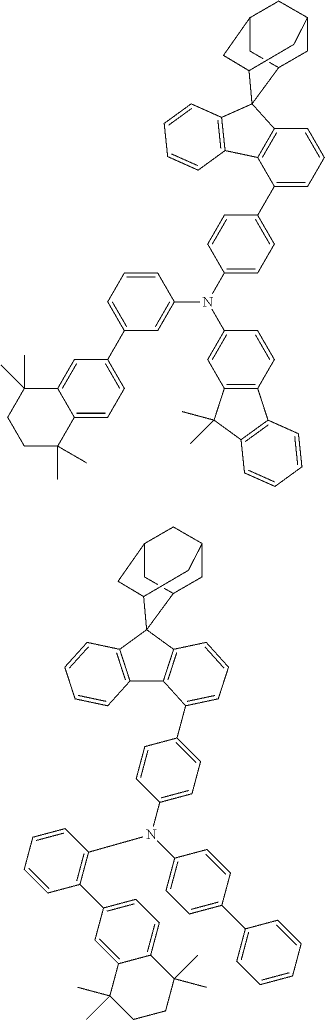 [SMILES: CC1(C)CCC(C)(C)c2cc(-c3cccc(N(c4ccc(-c5cccc6c5-c5ccccc5C65C6CC7CC(C6)CC5C7)cc4)c4ccc5c(c4)C(C)(C)c4ccccc4-5)c3)ccc21 ; CC1(C)CCC(C)(C)c2cc(-c3ccccc3N(c3ccc(-c4ccccc4)cc3)c3ccc(-c4cccc5c4-c4ccccc4C54C5CC6CC(C5)CC4C6)cc3)ccc21]

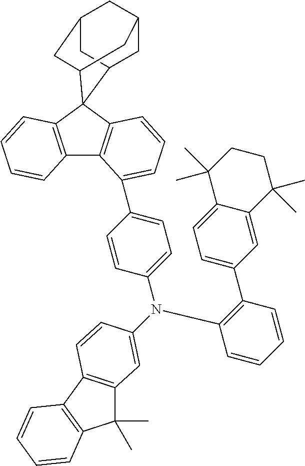 [SMILES: CC1(C)CCC(C)(C)c2cc(-c3ccccc3N(c3ccc(-c4cccc5c4-c4ccccc4C54C5CC6CC(C5)CC4C6)cc3)c3ccc4c(c3)C(C)(C)c3ccccc3-4)ccc21]

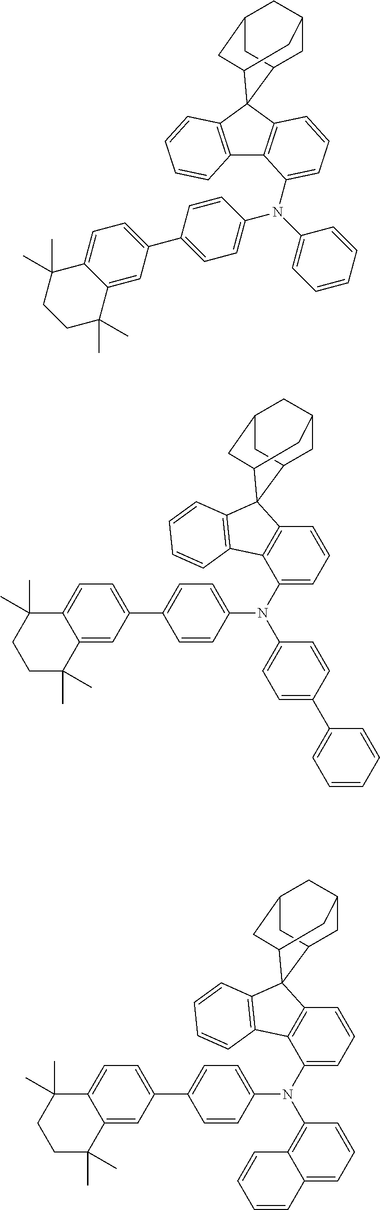 [SMILES: CC1(C)CCC(C)(C)c2cc(-c3ccc(N(c4ccccc4)c4cccc5c4-c4ccccc4C54C5CC6CC(C5)CC4C6)cc3)ccc21 ; CC1(C)CCC(C)(C)c2cc(-c3ccc(N(c4ccc(-c5ccccc5)cc4)c4cccc5c4-c4ccccc4C54C5CC6CC(C5)CC4C6)cc3)ccc21 ; CC1(C)CCC(C)(C)c2cc(-c3ccc(N(c4cccc5c4-c4ccccc4C54C5CC6CC(C5)CC4C6)c4cccc5ccccc45)cc3)ccc21]

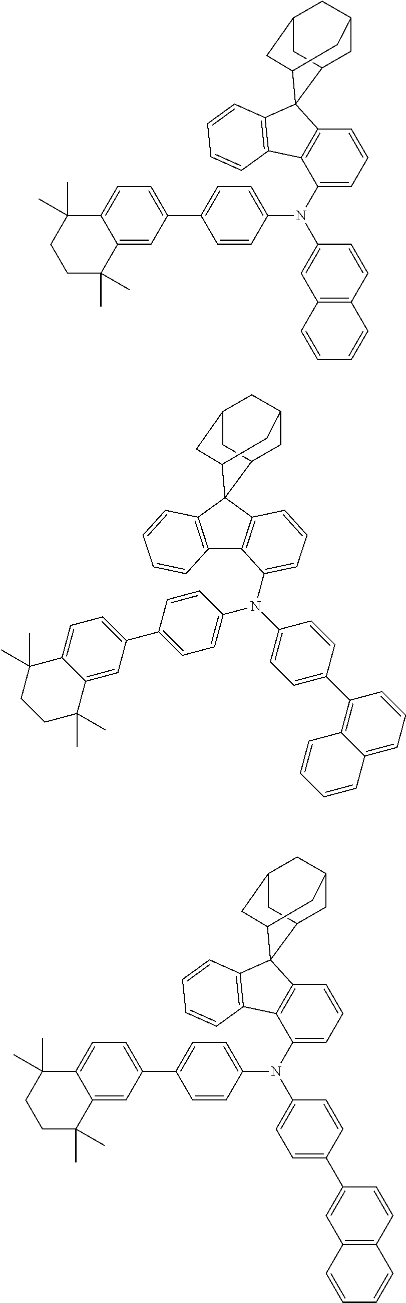 [SMILES: CC1(C)CCC(C)(C)c2cc(-c3ccc(N(c4ccc5ccccc5c4)c4cccc5c4-c4ccccc4C54C5CC6CC(C5)CC4C6)cc3)ccc21 ; CC1(C)CCC(C)(C)c2cc(-c3ccc(N(c4ccc(-c5cccc6ccccc56)cc4)c4cccc5c4-c4ccccc4C54C5CC6CC(C5)CC4C6)cc3)ccc21 ; CC1(C)CCC(C)(C)c2cc(-c3ccc(N(c4ccc(-c5ccc6ccccc6c5)cc4)c4cccc5c4-c4ccccc4C54C5CC6CC(C5)CC4C6)cc3)ccc21]

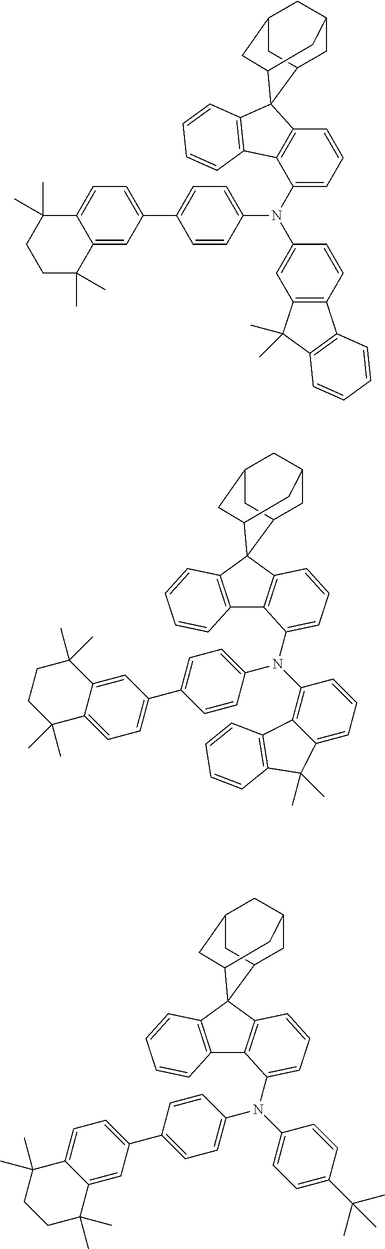 [SMILES: CC1(C)CCC(C)(C)c2cc(-c3ccc(N(c4ccc5c(c4)C(C)(C)c4ccccc4-5)c4cccc5c4-c4ccccc4C54C5CC6CC(C5)CC4C6)cc3)ccc21 ; CC1(C)CCC(C)(C)c2cc(-c3ccc(N(c4cccc5c4-c4ccccc4C5(C)C)c4cccc5c4-c4ccccc4C54C5CC6CC(C5)CC4C6)cc3)ccc21 ; CC(C)(C)c1ccc(N(c2ccc(-c3ccc4c(c3)C(C)(C)CCC4(C)C)cc2)c2cccc3c2-c2ccccc2C32C3CC4CC(C3)CC2C4)cc1]

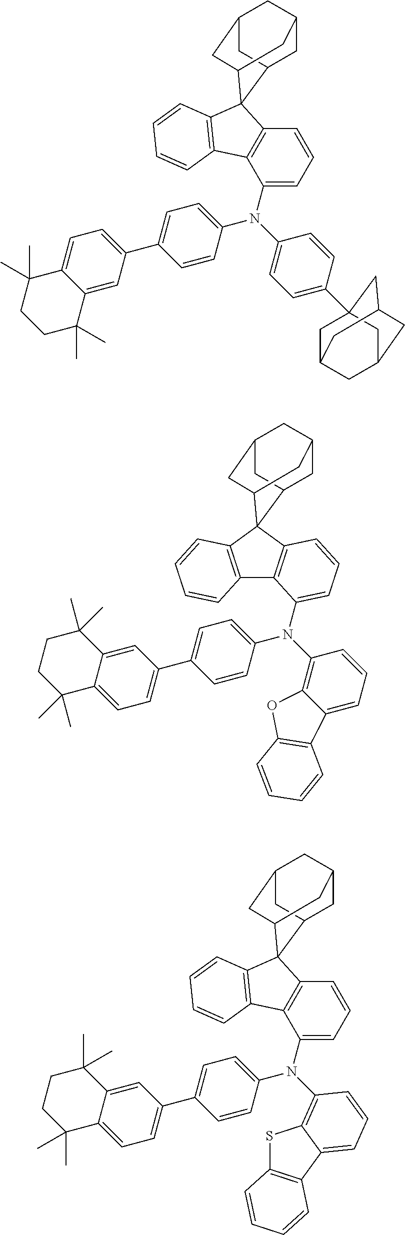 [SMILES: CC1(C)CCC(C)(C)c2cc(-c3ccc(N(c4ccc(C56CC7CC(CC(C7)C5)C6)cc4)c4cccc5c4-c4ccccc4C54C5CC6CC(C5)CC4C6)cc3)ccc21 ; CC1(C)CCC(C)(C)c2cc(-c3ccc(N(c4cccc5c4-c4ccccc4C54C5CC6CC(C5)CC4C6)c4cccc5c4oc4ccccc45)cc3)ccc21 ; CC1(C)CCC(C)(C)c2cc(-c3ccc(N(c4cccc5c4-c4ccccc4C54C5CC6CC(C5)CC4C6)c4cccc5c4sc4ccccc45)cc3)ccc21]

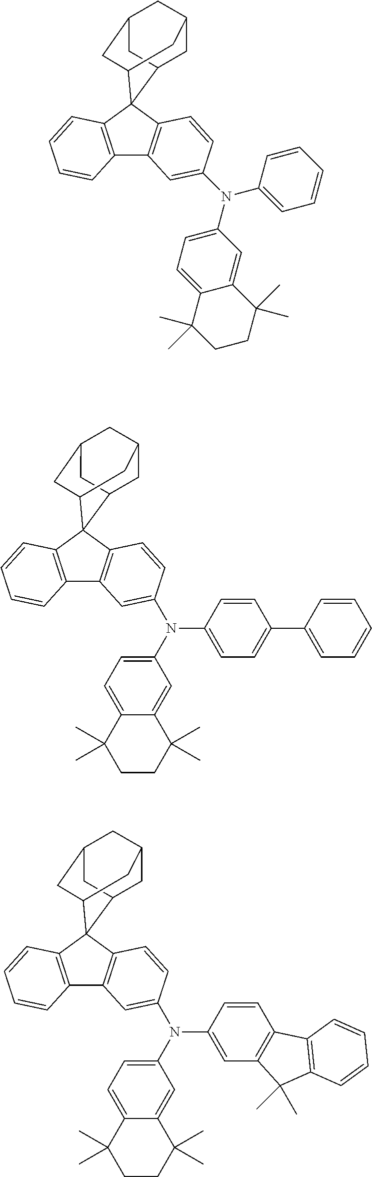 [SMILES: CC1(C)CCC(C)(C)c2cc(N(c3ccccc3)c3ccc4c(c3)-c3ccccc3C43C4CC5CC(C4)CC3C5)ccc21 ; CC1(C)CCC(C)(C)c2cc(N(c3ccc(-c4ccccc4)cc3)c3ccc4c(c3)-c3ccccc3C43C4CC5CC(C4)CC3C5)ccc21 ; CC1(C)CCC(C)(C)c2cc(N(c3ccc4c(c3)-c3ccccc3C43C4CC5CC(C4)CC3C5)c3ccc4c(c3)C(C)(C)c3ccccc3-4)ccc21]

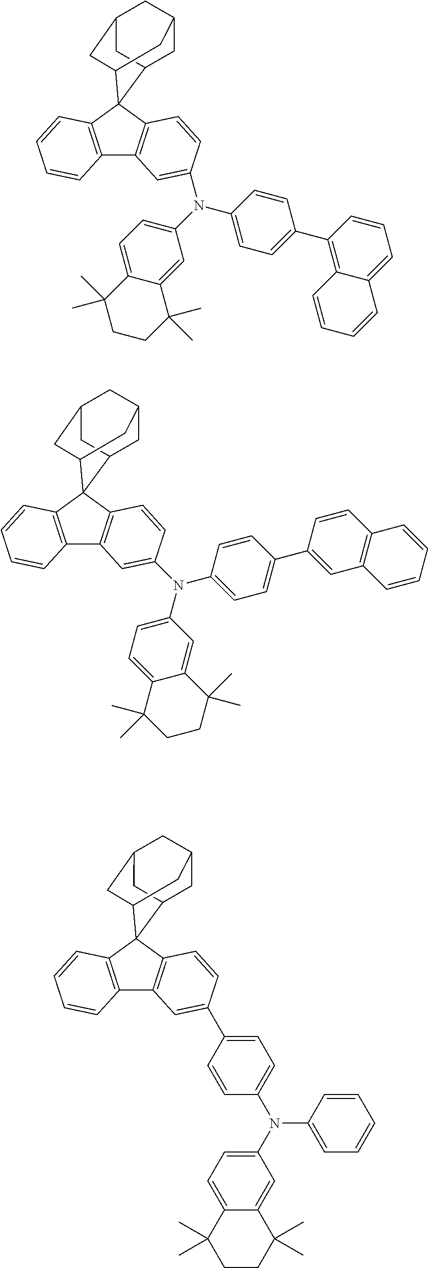 [SMILES: CC1(C)CCC(C)(C)c2cc(N(c3ccc(-c4cccc5ccccc45)cc3)c3ccc4c(c3)-c3ccccc3C43C4CC5CC(C4)CC3C5)ccc21 ; CC1(C)CCC(C)(C)c2cc(N(c3ccc(-c4ccc5ccccc5c4)cc3)c3ccc4c(c3)-c3ccccc3C43C4CC5CC(C4)CC3C5)ccc21 ; CC1(C)CCC(C)(C)c2cc(N(c3ccccc3)c3ccc(-c4ccc5c(c4)-c4ccccc4C54C5CC6CC(C5)CC4C6)cc3)ccc21]

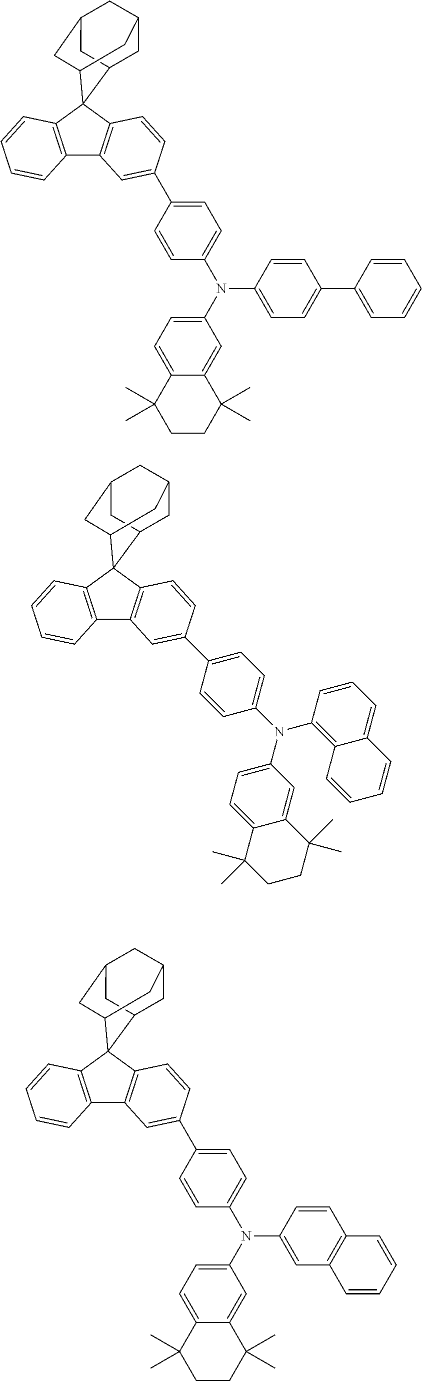

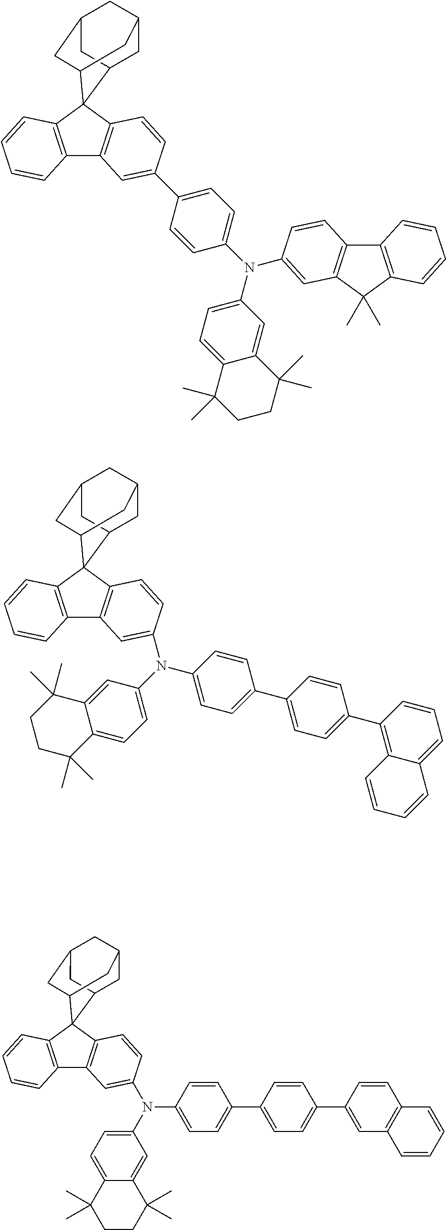 [SMILES: CC1(C)CCC(C)(C)c2cc(N(c3ccc(-c4ccc5c(c4)-c4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc(-c5cccc6ccccc56)cc4)cc3)c3ccc4c(c3)-c3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3ccc4c(c3)-c3ccccc3C43C4CC5CC(C4)CC3C5)ccc21]

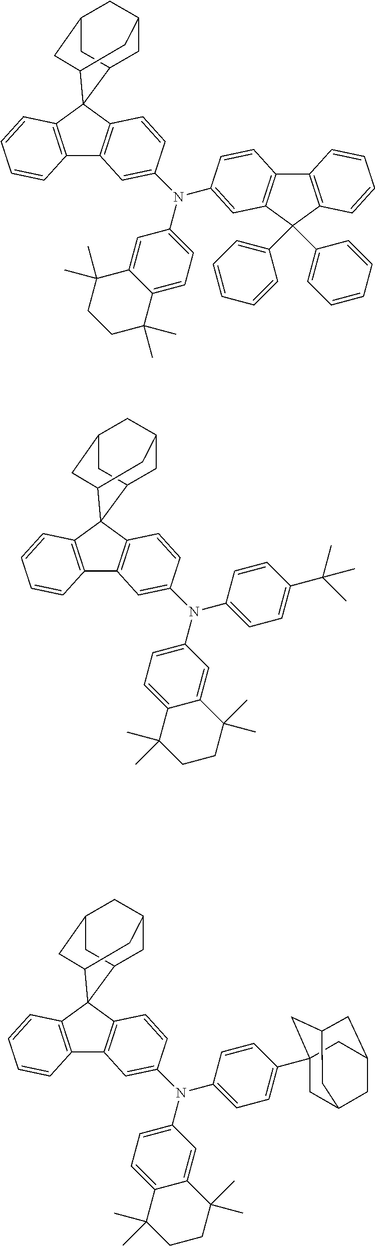 [SMILES: CC1(C)CCC(C)(C)c2cc(N(c3ccc4c(c3)-c3ccccc3C43C4CC5CC(C4)CC3C5)c3ccc4c(c3)C(c3ccccc3)(c3ccccc3)c3ccccc3-4)ccc21 ; CC(C)(C)c1ccc(N(c2ccc3c(c2)-c2ccccc2C32C3CC4CC(C3)CC2C4)c2ccc3c(c2)C(C)(C)CCC3(C)C)cc1 ; CC1(C)CCC(C)(C)c2cc(N(c3ccc(C45CC6CC(CC(C6)C4)C5)cc3)c3ccc4c(c3)-c3ccccc3C43C4CC5CC(C4)CC3C5)ccc21]

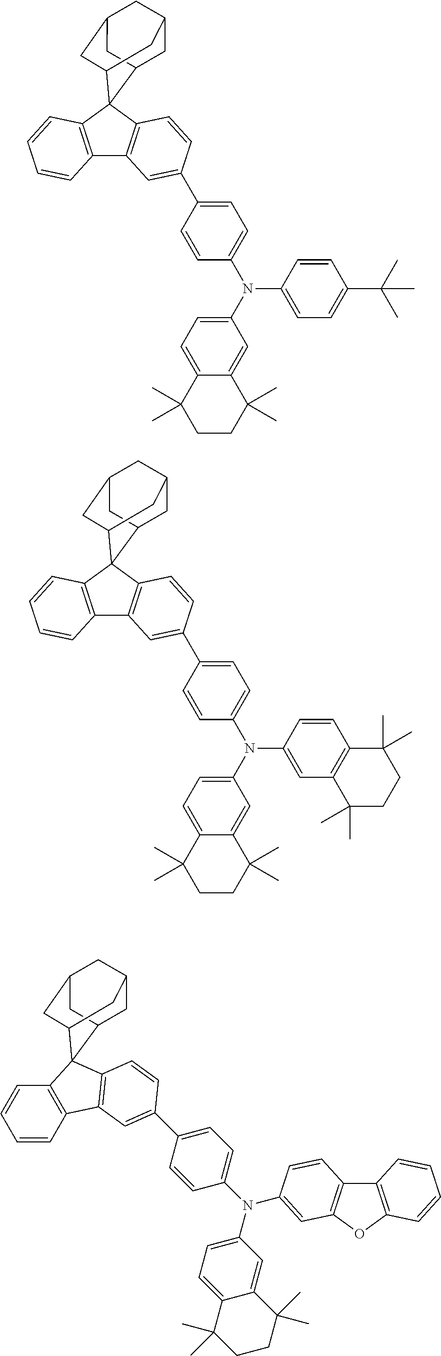

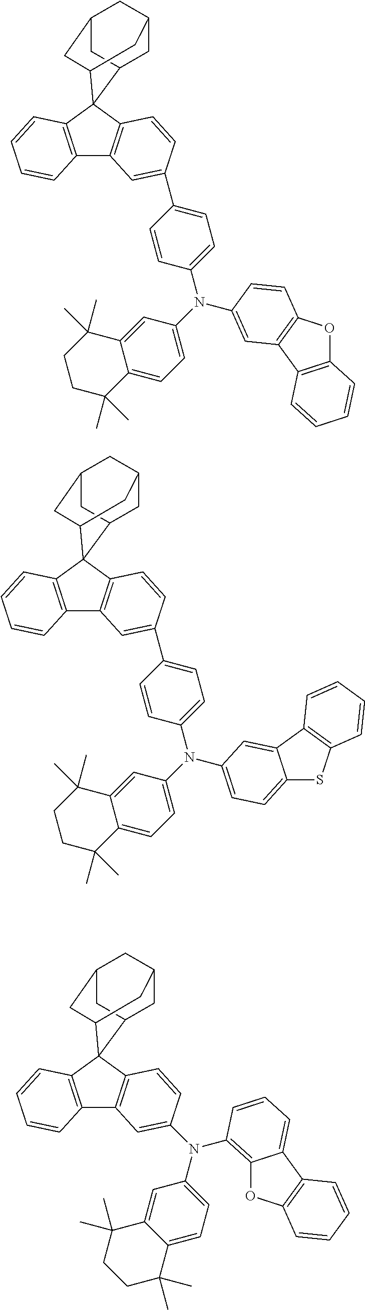

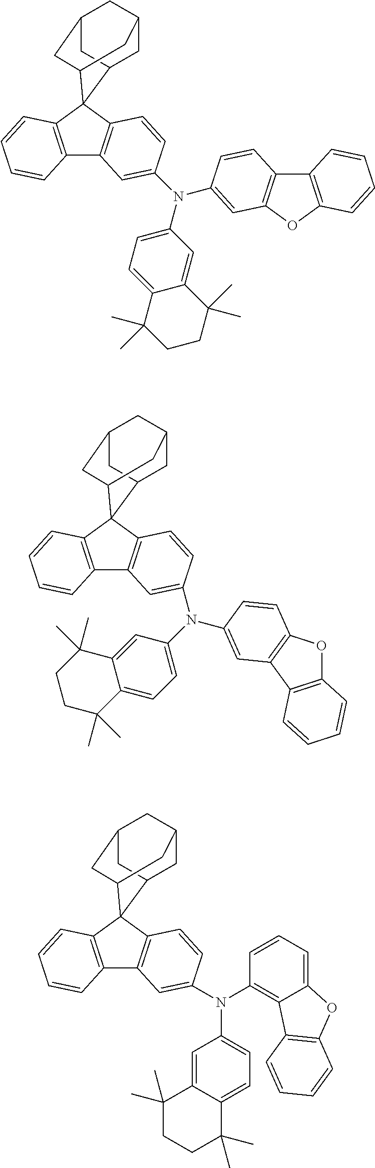 [SMILES: CC1(C)CCC(C)(C)c2cc(N(c3ccc4c(c3)-c3ccccc3C43C4CC5CC(C4)CC3C5)c3ccc4c(c3)oc3ccccc34)ccc21 ; CC1(C)CCC(C)(C)c2cc(N(c3ccc4c(c3)-c3ccccc3C43C4CC5CC(C4)CC3C5)c3ccc4oc5ccccc5c4c3)ccc21 ; CC1(C)CCC(C)(C)c2cc(N(c3ccc4c(c3)-c3ccccc3C43C4CC5CC(C4)CC3C5)c3cccc4oc5ccccc5c34)ccc21]

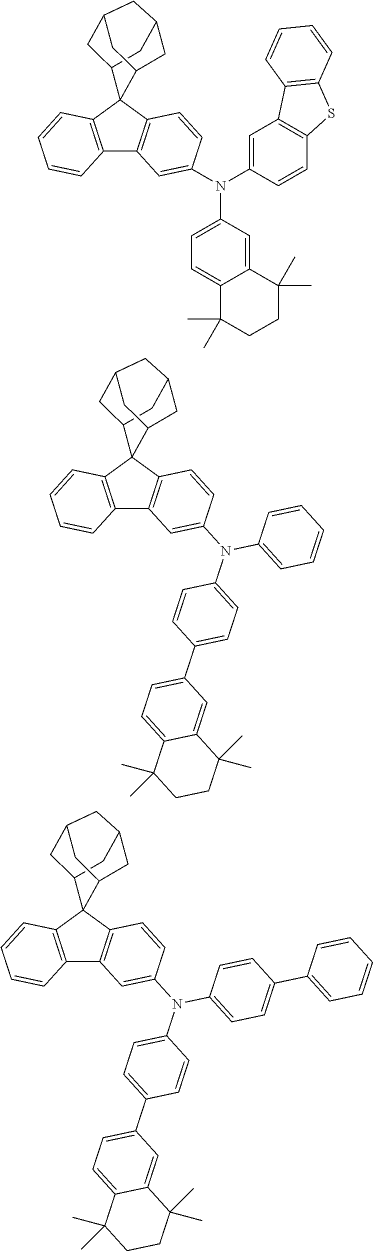

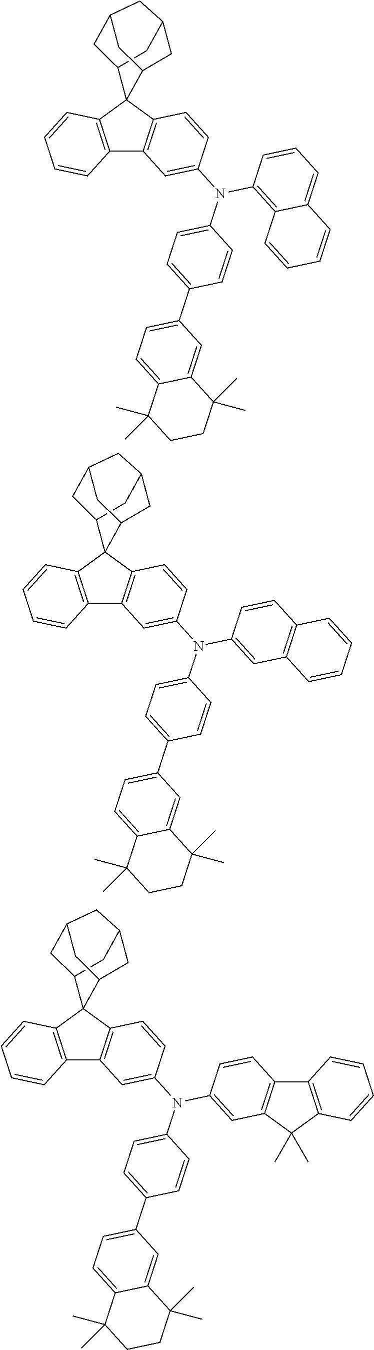

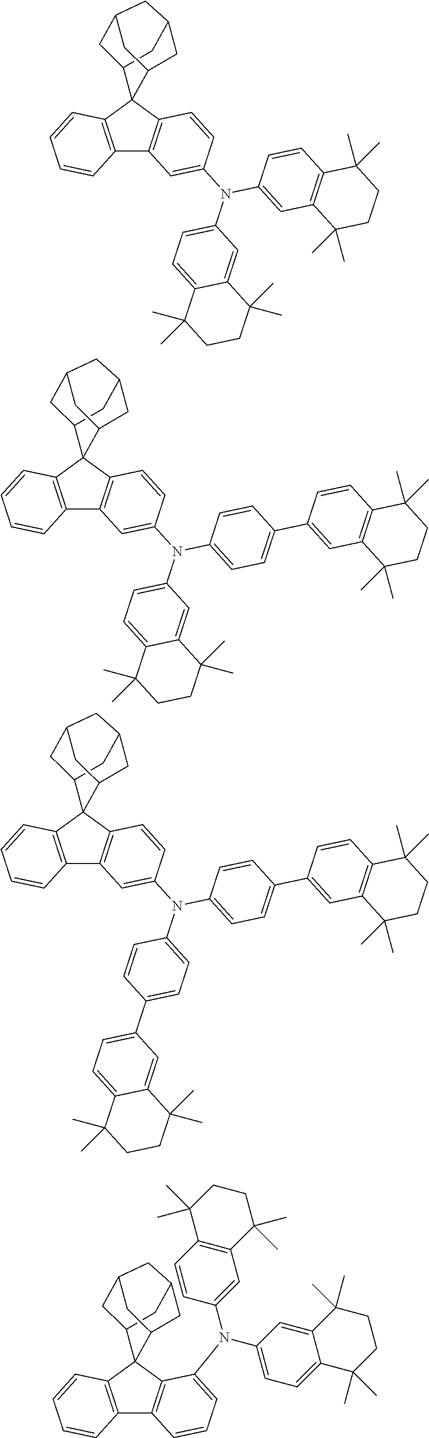

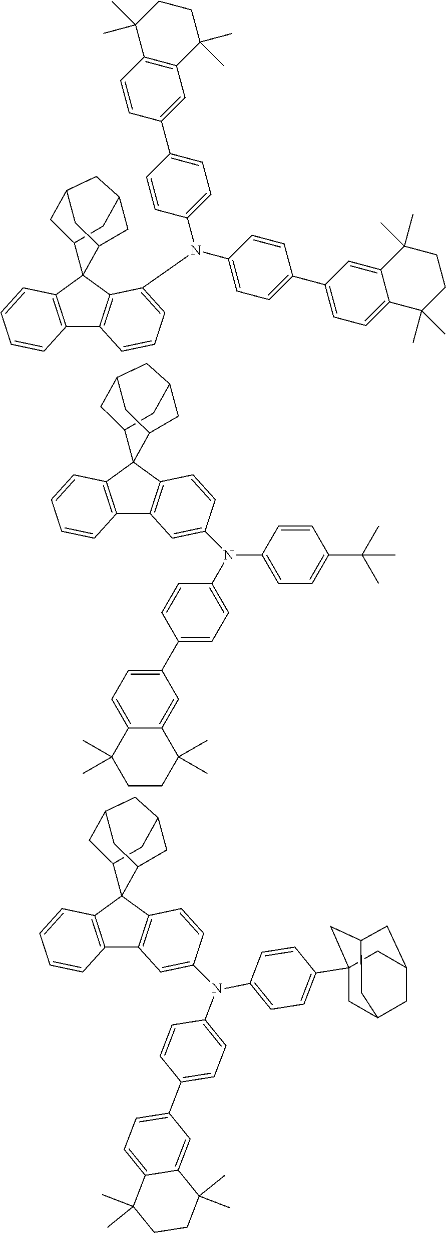

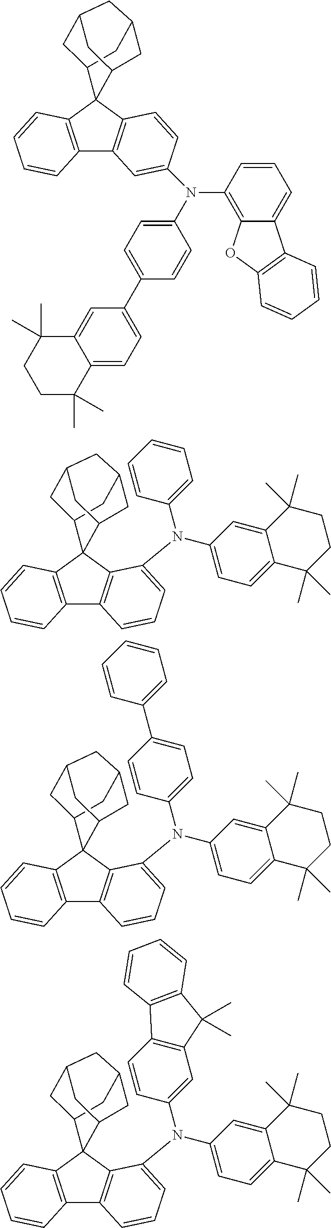

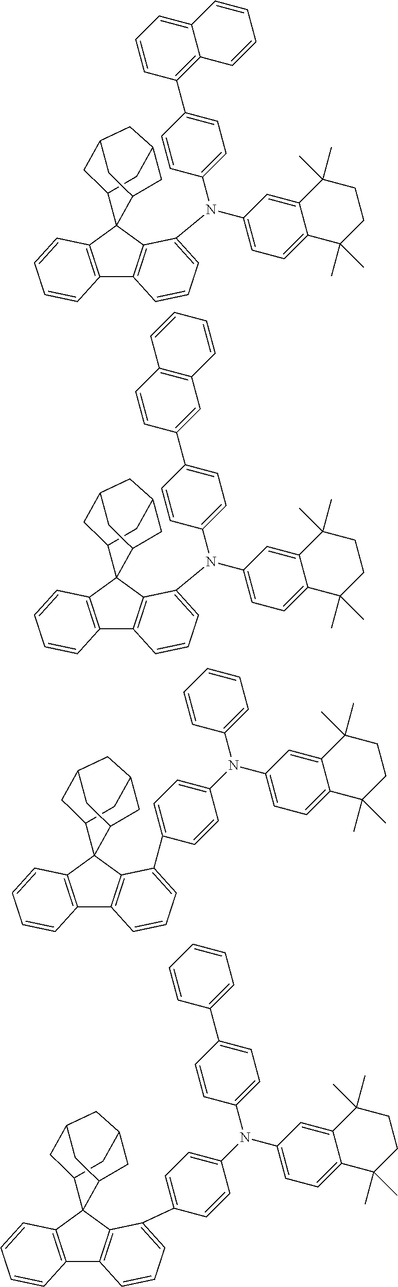

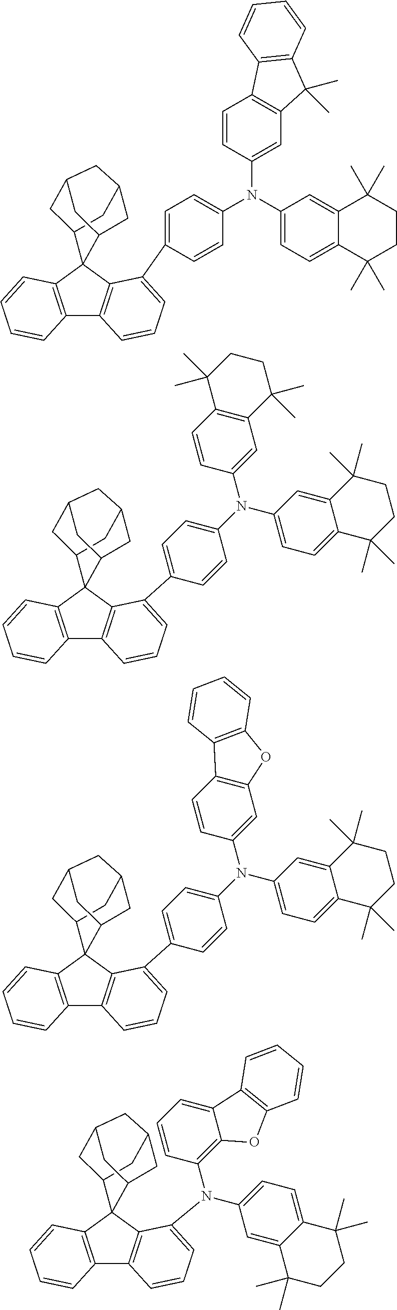

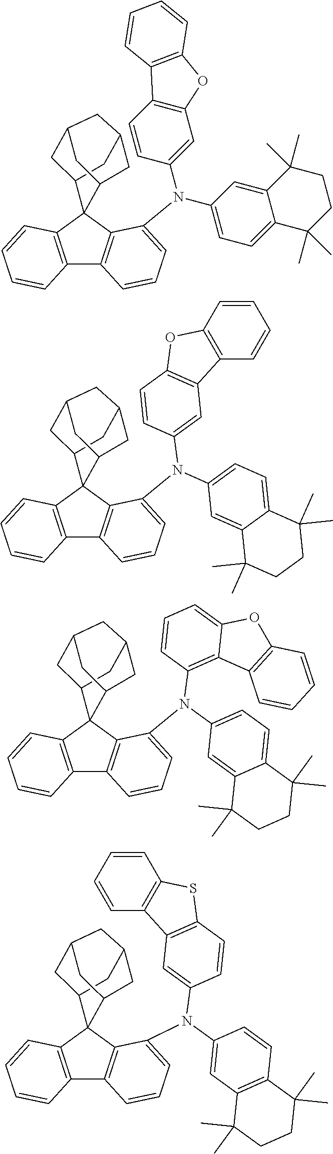

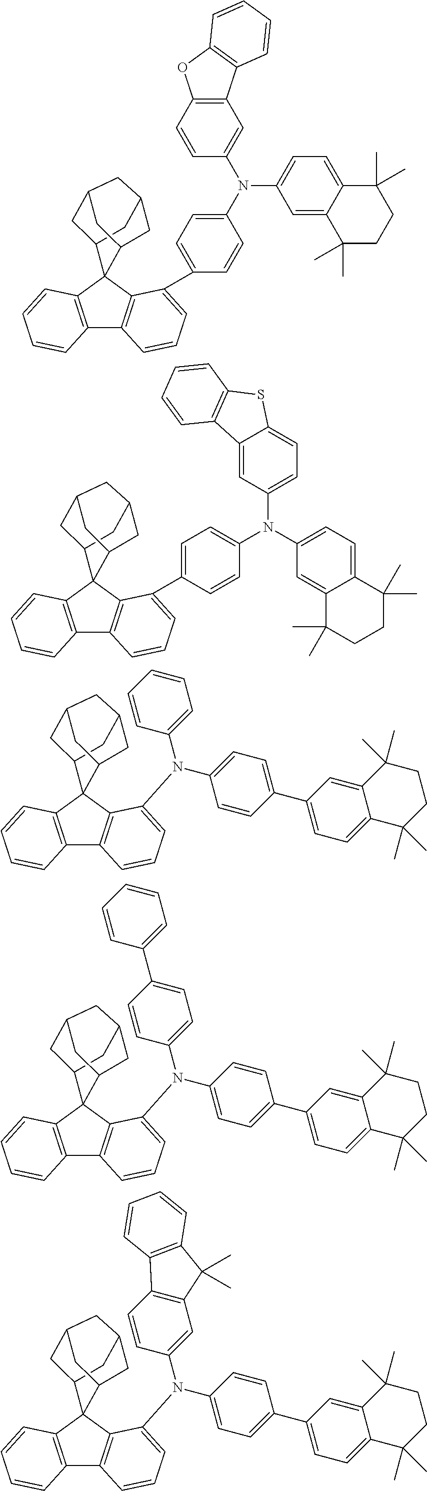

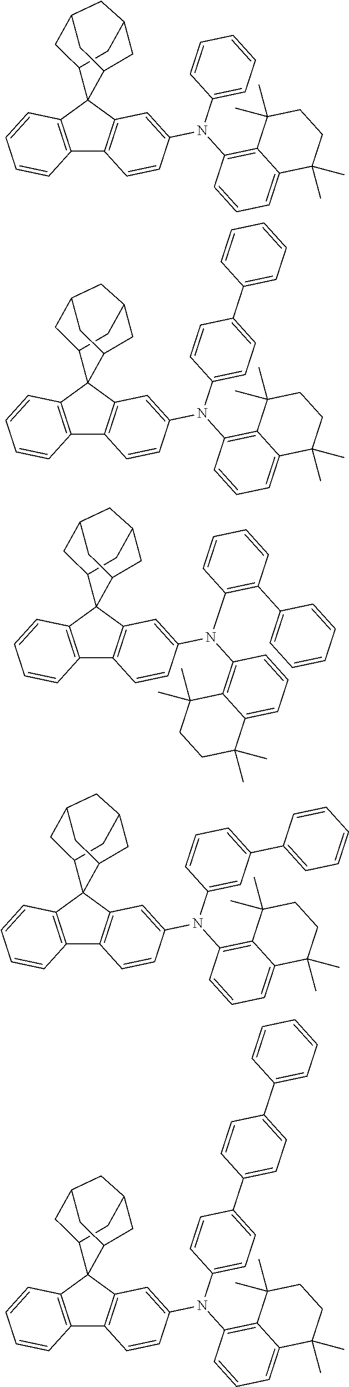

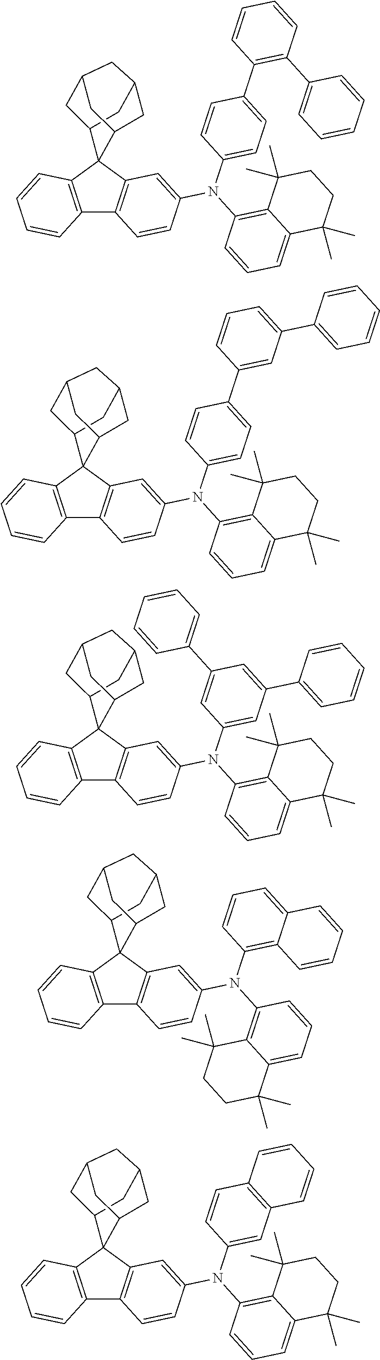

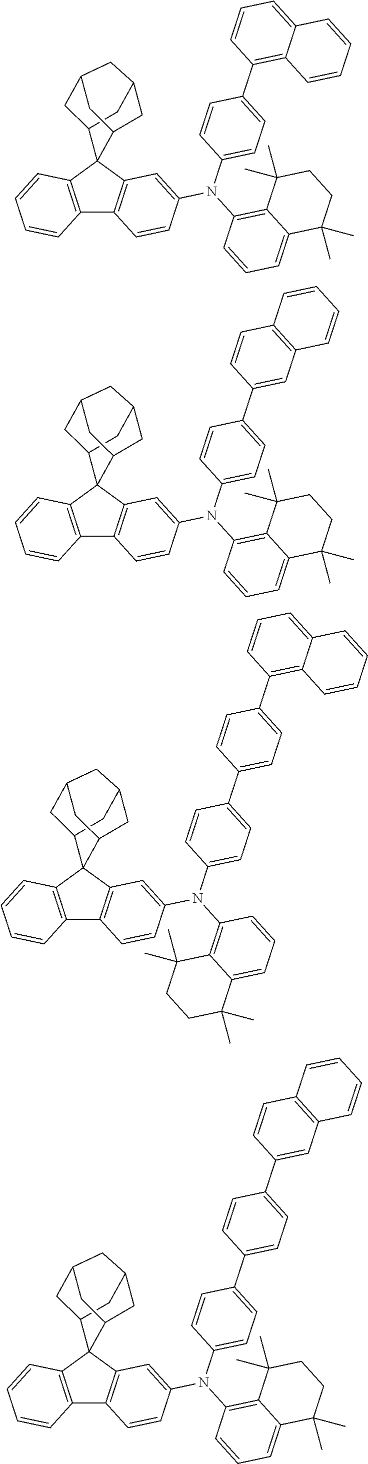

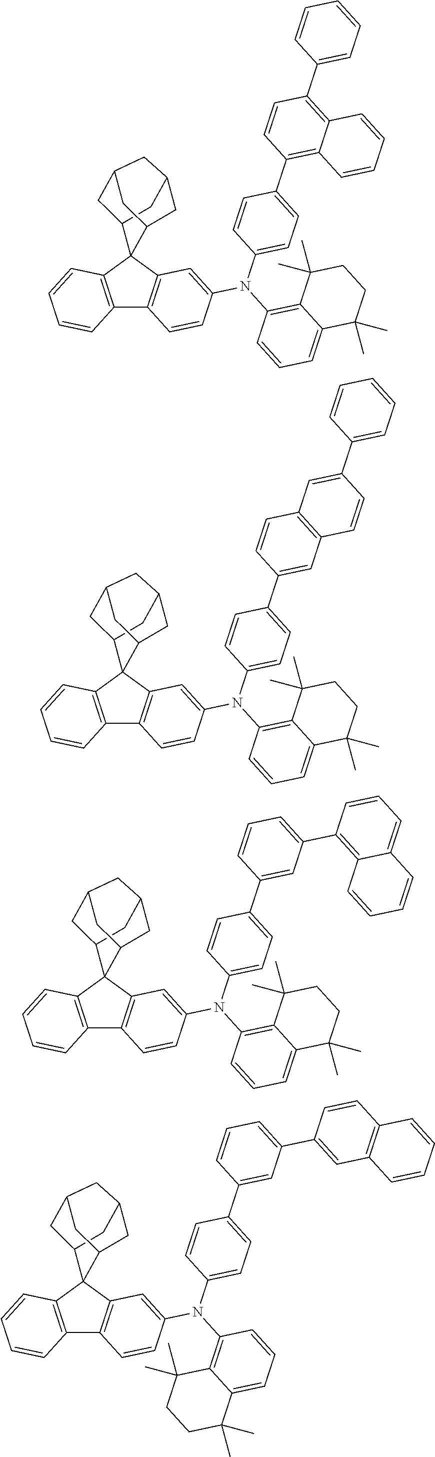

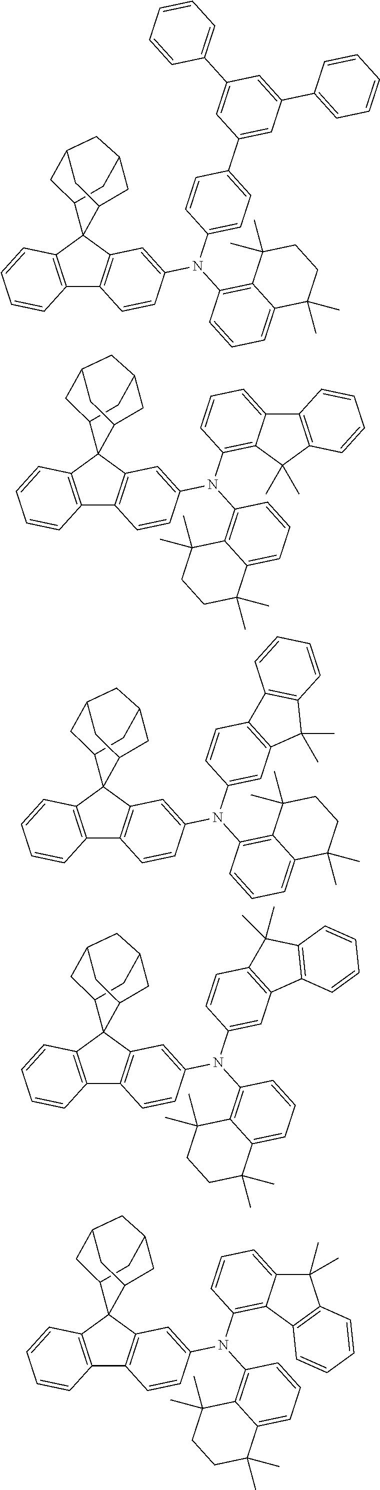

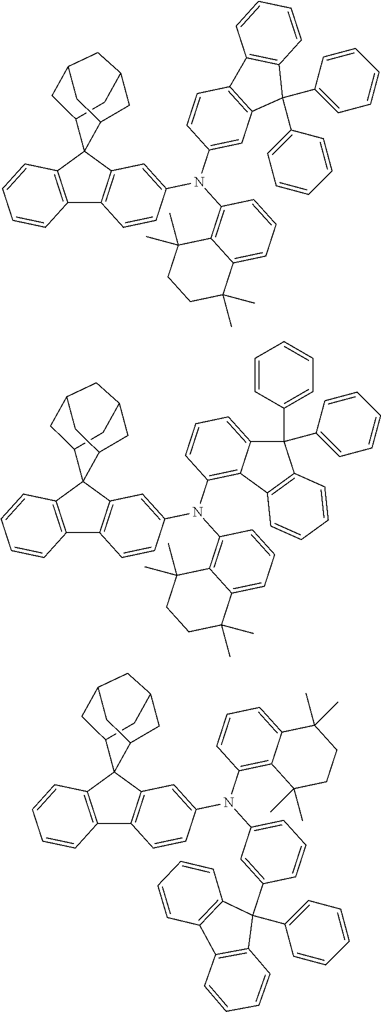

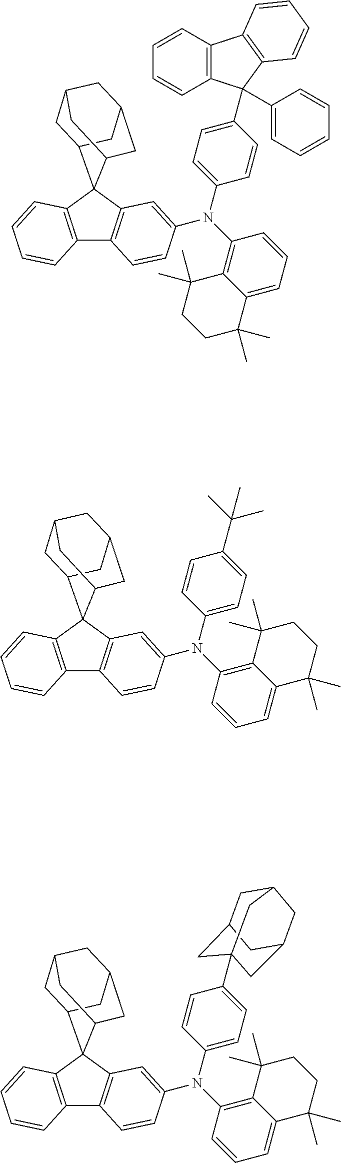 [SMILES: CC1(C)CCC(C)(C)c2c(N(c3ccc(C4(c5ccccc5)c5ccccc5-c5ccccc54)cc3)c3ccc4c(c3)C3(c5ccccc5-4)C4CC5CC(C4)CC3C5)cccc21 ; CC(C)(C)c1ccc(N(c2ccc3c(c2)C2(c4ccccc4-3)C3CC4CC(C3)CC2C4)c2cccc3c2C(C)(C)CCC3(C)C)cc1 ; CC1(C)CCC(C)(C)c2c(N(c3ccc(C45CC6CC(CC(C6)C4)C5)cc3)c3ccc4c(c3)C3(c5ccccc5-4)C4CC5CC(C4)CC3C5)cccc21]

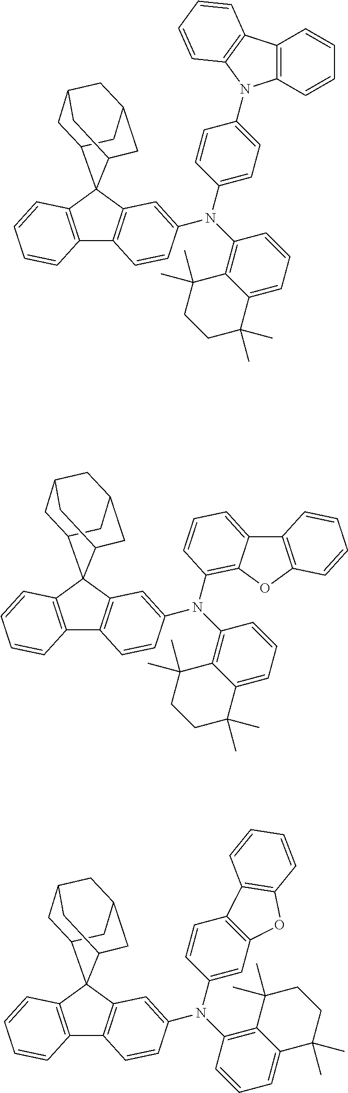 [SMILES: CC1(C)CCC(C)(C)c2c(N(c3ccc(-n4c5ccccc5c5ccccc54)cc3)c3ccc4c(c3)C3(c5ccccc5-4)C4CC5CC(C4)CC3C5)cccc21 ; CC1(C)CCC(C)(C)c2c(N(c3ccc4c(c3)C3(c5ccccc5-4)C4CC5CC(C4)CC3C5)c3cccc4c3oc3ccccc34)cccc21 ; CC1(C)CCC(C)(C)c2c(N(c3ccc4c(c3)C3(c5ccccc5-4)C4CC5CC(C4)CC3C5)c3ccc4c(c3)oc3ccccc34)cccc21]

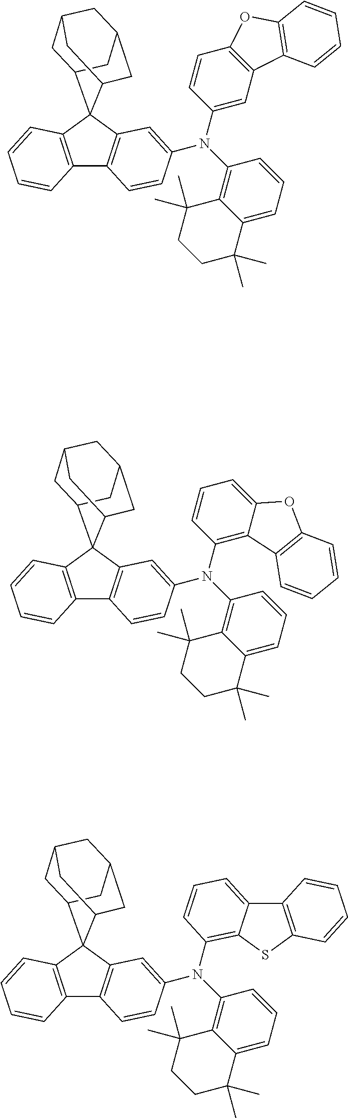 [SMILES: CC1(C)CCC(C)(C)c2c(N(c3ccc4c(c3)C3(c5ccccc5-4)C4CC5CC(C4)CC3C5)c3ccc4oc5ccccc5c4c3)cccc21 ; CC1(C)CCC(C)(C)c2c(N(c3ccc4c(c3)C3(c5ccccc5-4)C4CC5CC(C4)CC3C5)c3cccc4oc5ccccc5c34)cccc21 ; CC1(C)CCC(C)(C)c2c(N(c3ccc4c(c3)C3(c5ccccc5-4)C4CC5CC(C4)CC3C5)c3cccc4c3sc3ccccc34)cccc21]

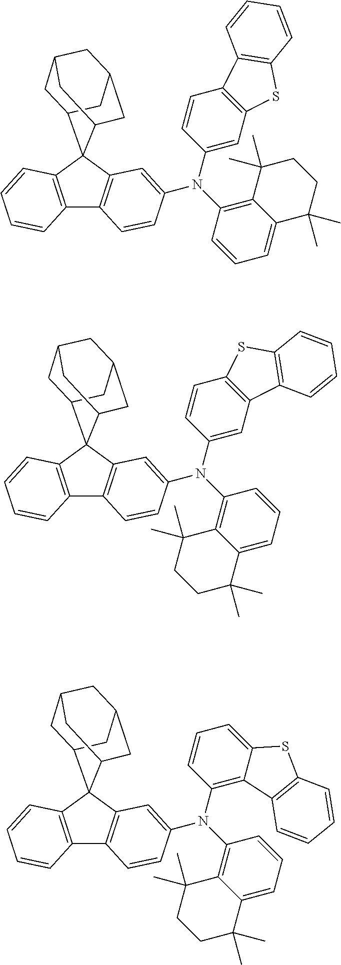 [SMILES: CC1(C)CCC(C)(C)c2c(N(c3ccc4c(c3)C3(c5ccccc5-4)C4CC5CC(C4)CC3C5)c3ccc4c(c3)sc3ccccc34)cccc21 ; CC1(C)CCC(C)(C)c2c(N(c3ccc4c(c3)C3(c5ccccc5-4)C4CC5CC(C4)CC3C5)c3ccc4sc5ccccc5c4c3)cccc21 ; CC1(C)CCC(C)(C)c2c(N(c3ccc4c(c3)C3(c5ccccc5-4)C4CC5CC(C4)CC3C5)c3cccc4sc5ccccc5c34)cccc21]

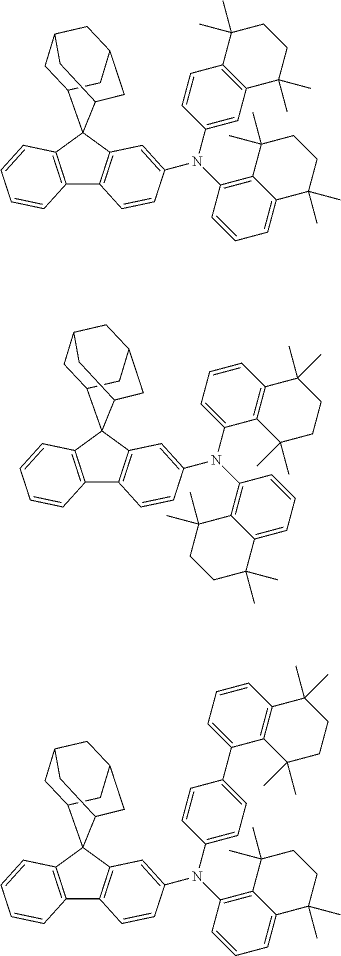 [SMILES: CC1(C)CCC(C)(C)c2cc(N(c3ccc4c(c3)C3(c5ccccc5-4)C4CC5CC(C4)CC3C5)c3cccc4c3C(C)(C)CCC4(C)C)ccc21 ; CC1(C)CCC(C)(C)c2c(N(c3ccc4c(c3)C3(c5ccccc5-4)C4CC5CC(C4)CC3C5)c3cccc4c3C(C)(C)CCC4(C)C)cccc21 ; CC1(C)CCC(C)(C)c2c(-c3ccc(N(c4ccc5c(c4)C4(c6ccccc6-5)C5CC6CC(C5)CC4C6)c4cccc5c4C(C)(C)CCC5(C)C)cc3)cccc21]

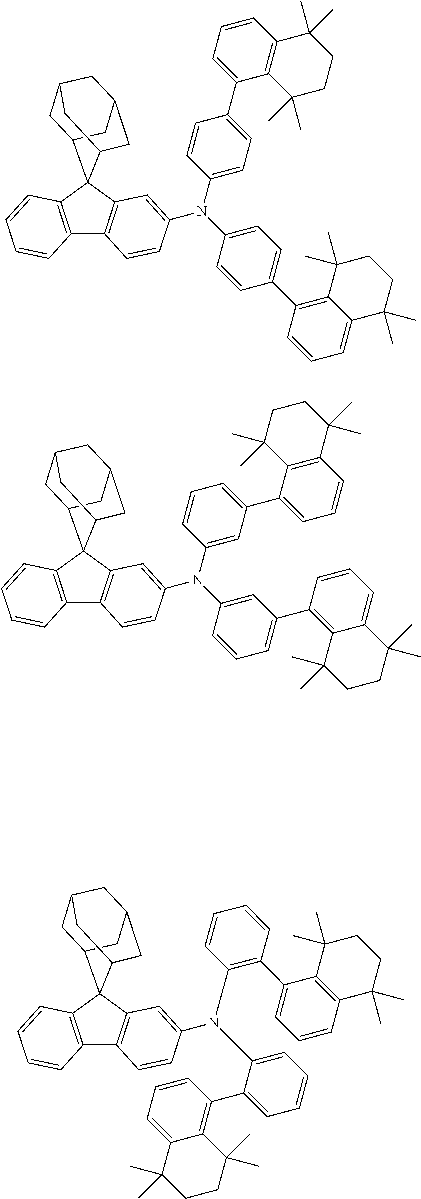

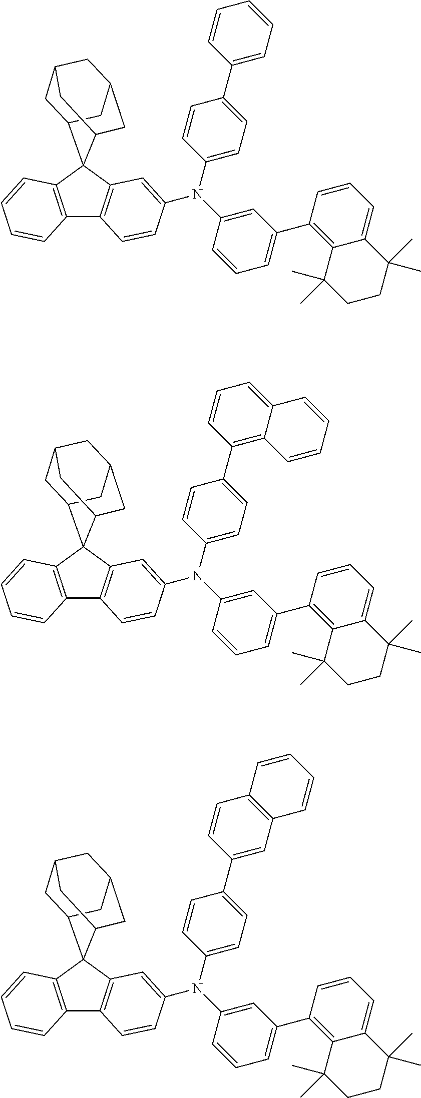 [SMILES: CC1(C)CCC(C)(C)c2c(-c3cccc(N(c4ccc(-c5ccccc5)cc4)c4ccc5c(c4)C4(c6ccccc6-5)C5CC6CC(C5)CC4C6)c3)cccc21 ; CC1(C)CCC(C)(C)c2c(-c3cccc(N(c4ccc(-c5cccc6ccccc56)cc4)c4ccc5c(c4)C4(c6ccccc6-5)C5CC6CC(C5)CC4C6)c3)cccc21 ; CC1(C)CCC(C)(C)c2c(-c3cccc(N(c4ccc(-c5ccc6ccccc6c5)cc4)c4ccc5c(c4)C4(c6ccccc6-5)C5CC6CC(C5)CC4C6)c3)cccc21]

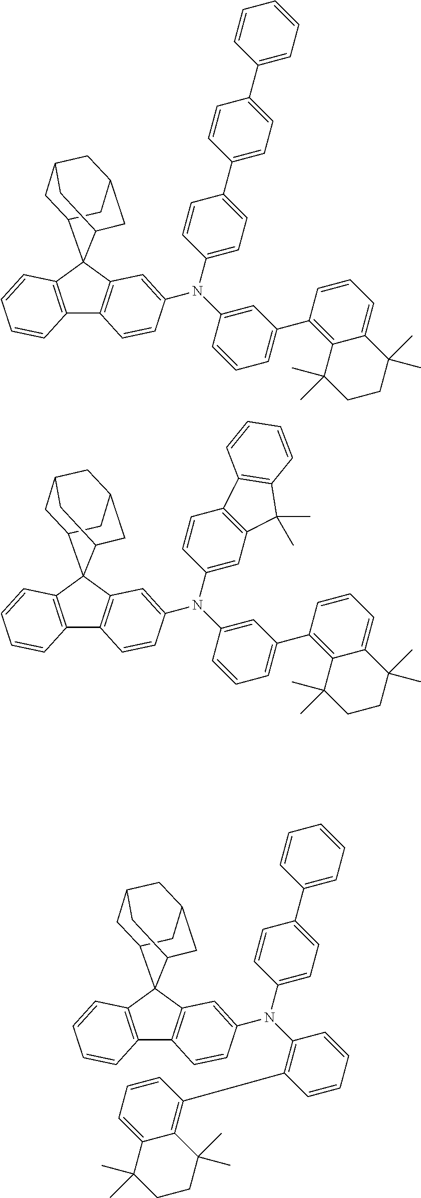

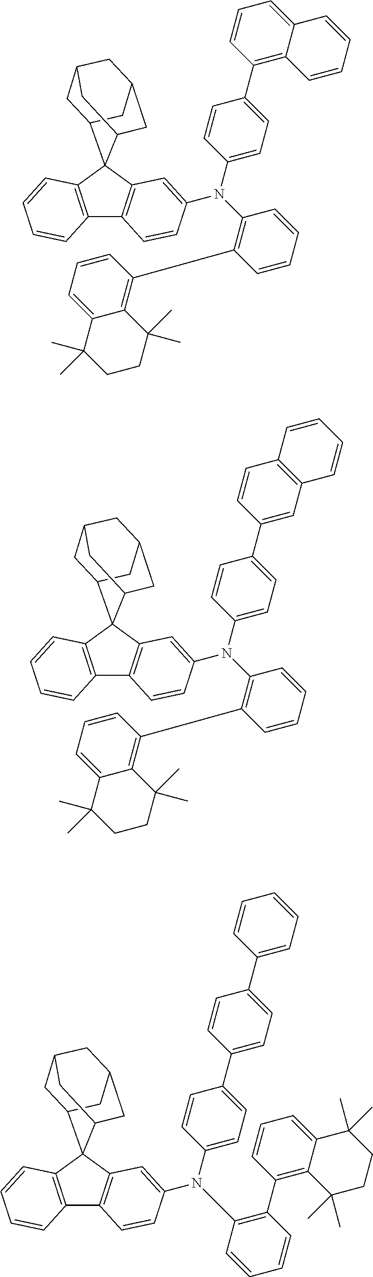 [SMILES: CC1(C)CCC(C)(C)c2c(-c3ccccc3N(c3ccc(-c4cccc5ccccc45)cc3)c3ccc4c(c3)C3(c5ccccc5-4)C4CC5CC(C4)CC3C5)cccc21 ; CC1(C)CCC(C)(C)c2c(-c3ccccc3N(c3ccc(-c4ccc5ccccc5c4)cc3)c3ccc4c(c3)C3(c5ccccc5-4)C4CC5CC(C4)CC3C5)cccc21 ; CC1(C)CCC(C)(C)c2c(-c3ccccc3N(c3ccc(-c4ccc(-c5ccccc5)cc4)cc3)c3ccc4c(c3)C3(c5ccccc5-4)C4CC5CC(C4)CC3C5)cccc21]

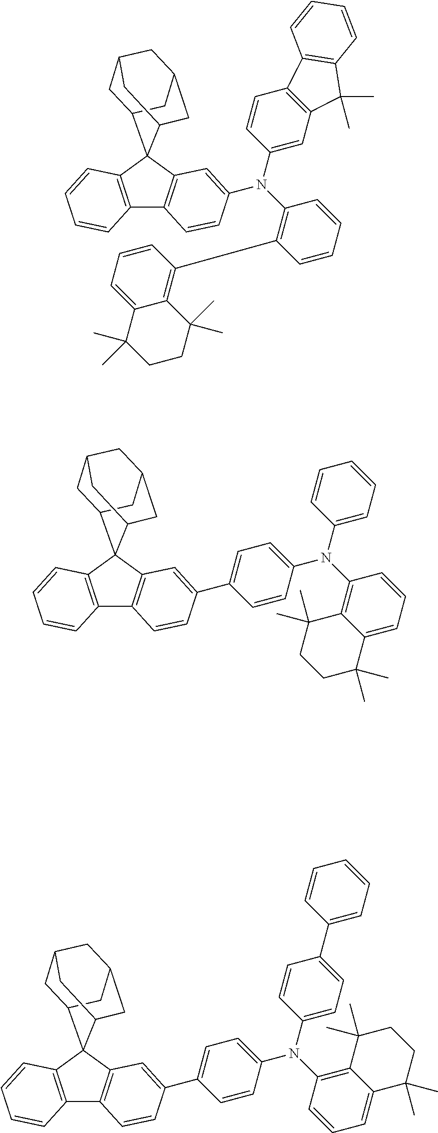 [SMILES: CC1(C)CCC(C)(C)c2c(-c3ccccc3N(c3ccc4c(c3)C(C)(C)c3ccccc3-4)c3ccc4c(c3)C3(c5ccccc5-4)C4CC5CC(C4)CC3C5)cccc21 ; CC1(C)CCC(C)(C)c2c(N(c3ccccc3)c3ccc(-c4ccc5c(c4)C4(c6ccccc6-5)C5CC6CC(C5)CC4C6)cc3)cccc21 ; CC1(C)CCC(C)(C)c2c(N(c3ccc(-c4ccccc4)cc3)c3ccc(-c4ccc5c(c4)C4(c6ccccc6-5)C5CC6CC(C5)CC4C6)cc3)cccc21]

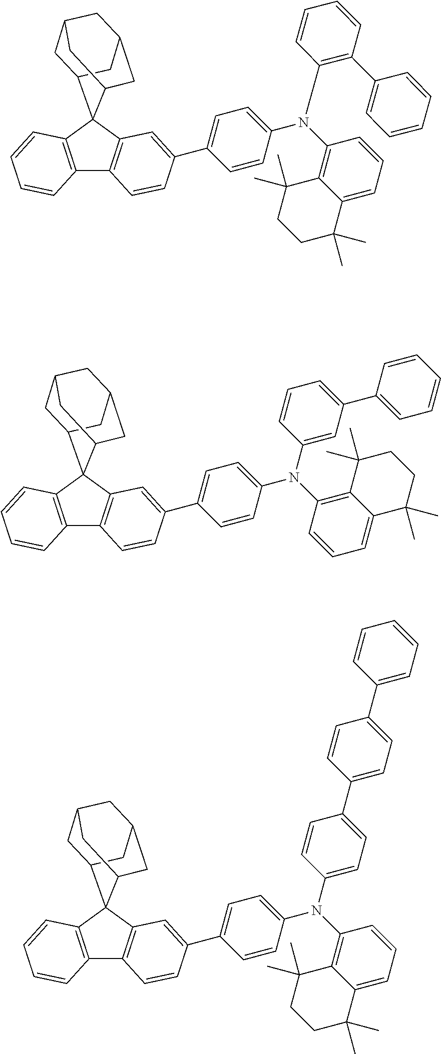 [SMILES: CC1(C)CCC(C)(C)c2c(N(c3ccc(-c4ccc5c(c4)C4(c6ccccc6-5)C5CC6CC(C5)CC4C6)cc3)c3ccccc3-c3ccccc3)cccc21 ; CC1(C)CCC(C)(C)c2c(N(c3ccc(-c4ccc5c(c4)C4(c6ccccc6-5)C5CC6CC(C5)CC4C6)cc3)c3cccc(-c4ccccc4)c3)cccc21 ; CC1(C)CCC(C)(C)c2c(N(c3ccc(-c4ccc(-c5ccccc5)cc4)cc3)c3ccc(-c4ccc5c(c4)C4(c6ccccc6-5)C5CC6CC(C5)CC4C6)cc3)cccc21]

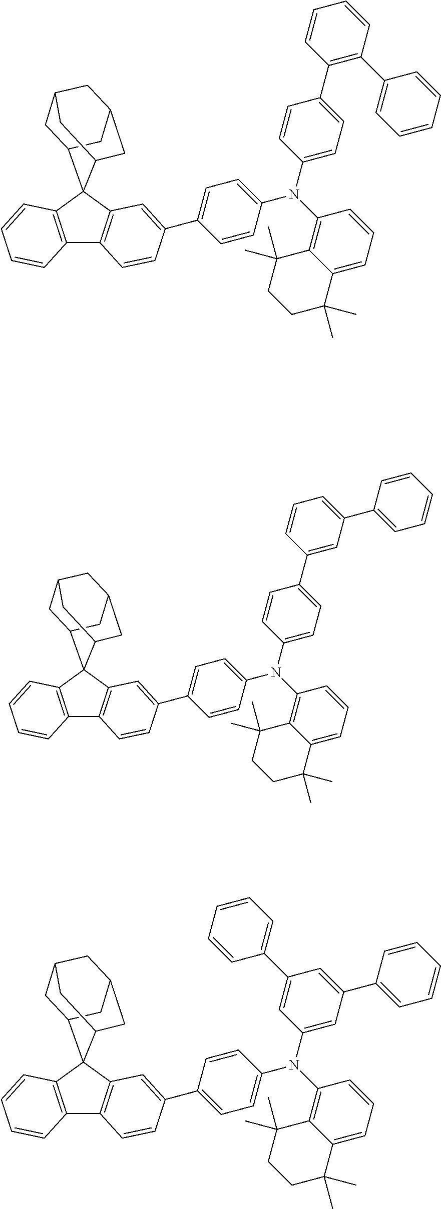 [SMILES: CC1(C)CCC(C)(C)c2c(N(c3ccc(-c4ccc5c(c4)C4(c6ccccc6-5)C5CC6CC(C5)CC4C6)cc3)c3ccc(-c4ccccc4-c4ccccc4)cc3)cccc21 ; CC1(C)CCC(C)(C)c2c(N(c3ccc(-c4cccc(-c5ccccc5)c4)cc3)c3ccc(-c4ccc5c(c4)C4(c6ccccc6-5)C5CC6CC(C5)CC4C6)cc3)cccc21 ; CC1(C)CCC(C)(C)c2c(N(c3ccc(-c4ccc5c(c4)C4(c6ccccc6-5)C5CC6CC(C5)CC4C6)cc3)c3cc(-c4ccccc4)cc(-c4ccccc4)c3)cccc21]

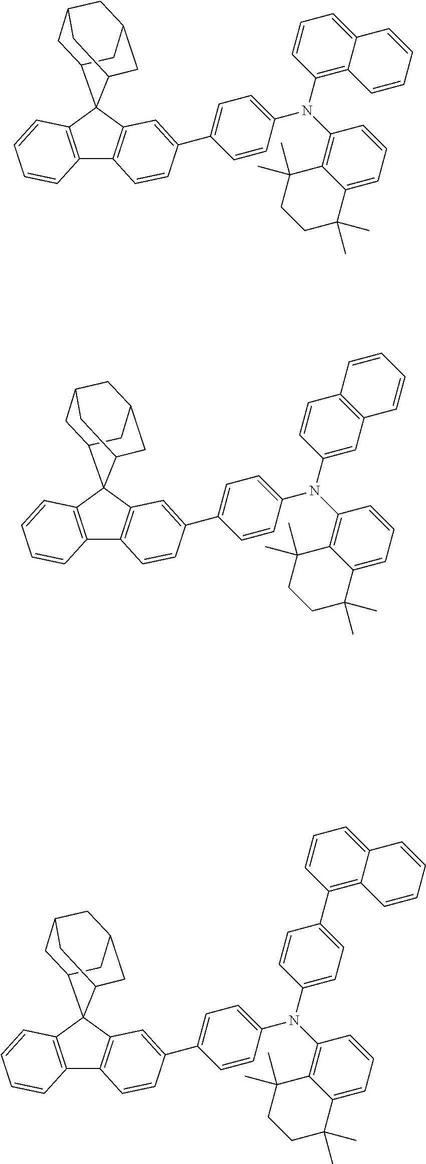 [SMILES: CC1(C)CCC(C)(C)c2c(N(c3ccc(-c4ccc5c(c4)C4(c6ccccc6-5)C5CC6CC(C5)CC4C6)cc3)c3cccc4ccccc34)cccc21 ; CC1(C)CCC(C)(C)c2c(N(c3ccc(-c4ccc5c(c4)C4(c6ccccc6-5)C5CC6CC(C5)CC4C6)cc3)c3ccc4ccccc4c3)cccc21 ; CC1(C)CCC(C)(C)c2c(N(c3ccc(-c4ccc5c(c4)C4(c6ccccc6-5)C5CC6CC(C5)CC4C6)cc3)c3ccc(-c4cccc5ccccc45)cc3)cccc21]

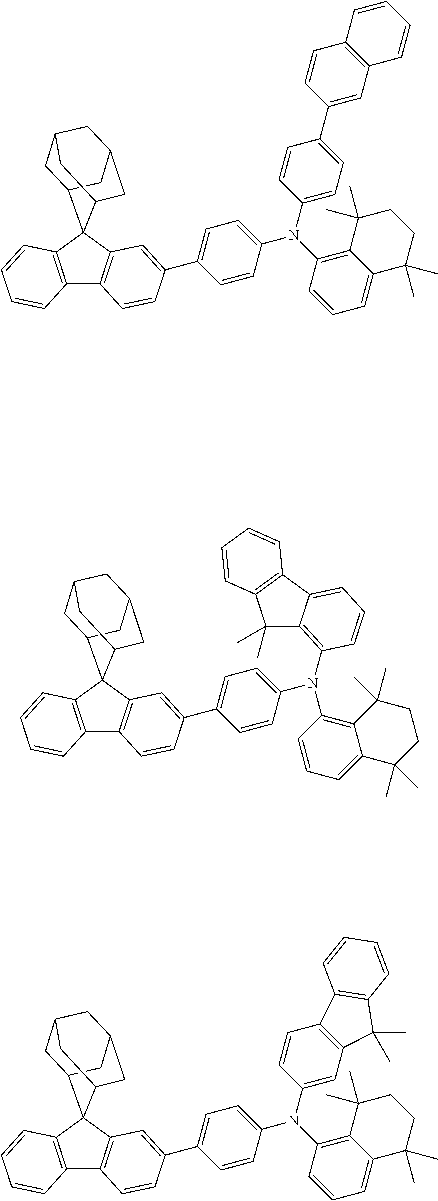 [SMILES: CC1(C)CCC(C)(C)c2c(N(c3ccc(-c4ccc5c(c4)C4(c6ccccc6-5)C5CC6CC(C5)CC4C6)cc3)c3ccc(-c4ccc5ccccc5c4)cc3)cccc21 ; CC1(C)CCC(C)(C)c2c(N(c3ccc(-c4ccc5c(c4)C4(c6ccccc6-5)C5CC6CC(C5)CC4C6)cc3)c3cccc4c3C(C)(C)c3ccccc3-4)cccc21 ; CC1(C)CCC(C)(C)c2c(N(c3ccc(-c4ccc5c(c4)C4(c6ccccc6-5)C5CC6CC(C5)CC4C6)cc3)c3ccc4c(c3)C(C)(C)c3ccccc3-4)cccc21]

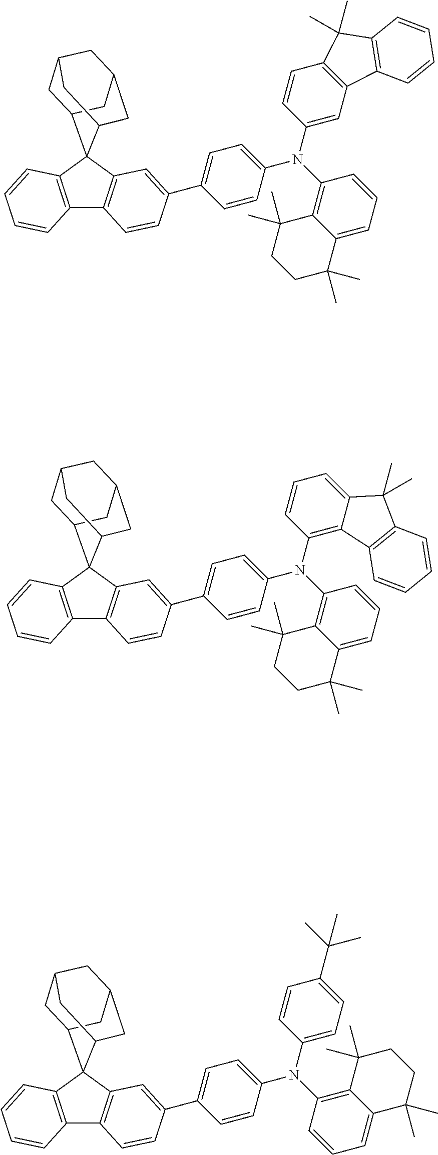 [SMILES: CC1(C)CCC(C)(C)c2c(N(c3ccc(-c4ccc5c(c4)C4(c6ccccc6-5)C5CC6CC(C5)CC4C6)cc3)c3ccc4c(c3)-c3ccccc3C4(C)C)cccc21 ; CC1(C)CCC(C)(C)c2c(N(c3ccc(-c4ccc5c(c4)C4(c6ccccc6-5)C5CC6CC(C5)CC4C6)cc3)c3cccc4c3-c3ccccc3C4(C)C)cccc21 ; CC(C)(C)c1ccc(N(c2ccc(-c3ccc4c(c3)C3(c5ccccc5-4)C4CC5CC(C4)CC3C5)cc2)c2cccc3c2C(C)(C)CCC3(C)C)cc1]

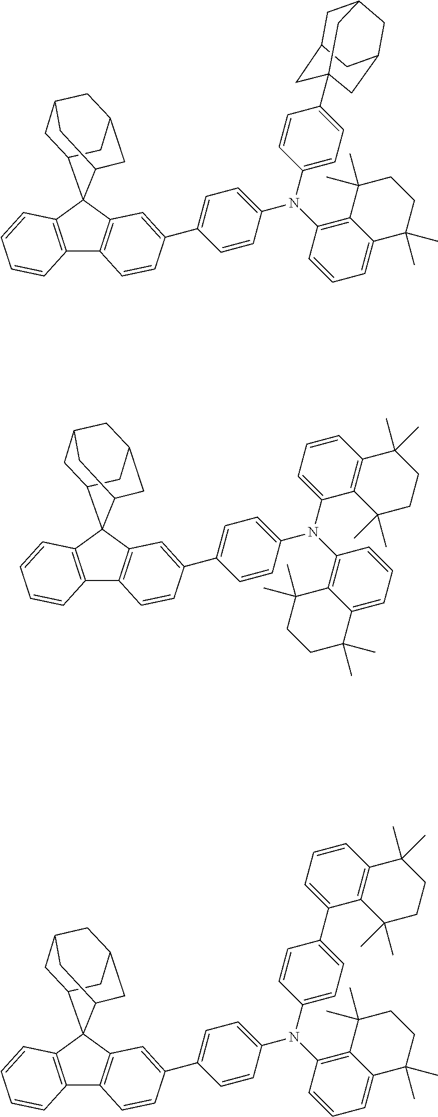 [SMILES: CC1(C)CCC(C)(C)c2c(N(c3ccc(-c4ccc5c(c4)C4(c6ccccc6-5)C5CC6CC(C5)CC4C6)cc3)c3ccc(C45CC6CC(CC(C6)C4)C5)cc3)cccc21 ; CC1(C)CCC(C)(C)c2c(N(c3ccc(-c4ccc5c(c4)C4(c6ccccc6-5)C5CC6CC(C5)CC4C6)cc3)c3cccc4c3C(C)(C)CCC4(C)C)cccc21 ; CC1(C)CCC(C)(C)c2c(-c3ccc(N(c4ccc(-c5ccc6c(c5)C5(c7ccccc7-6)C6CC7CC(C6)CC5C7)cc4)c4cccc5c4C(C)(C)CCC5(C)C)cc3)cccc21]

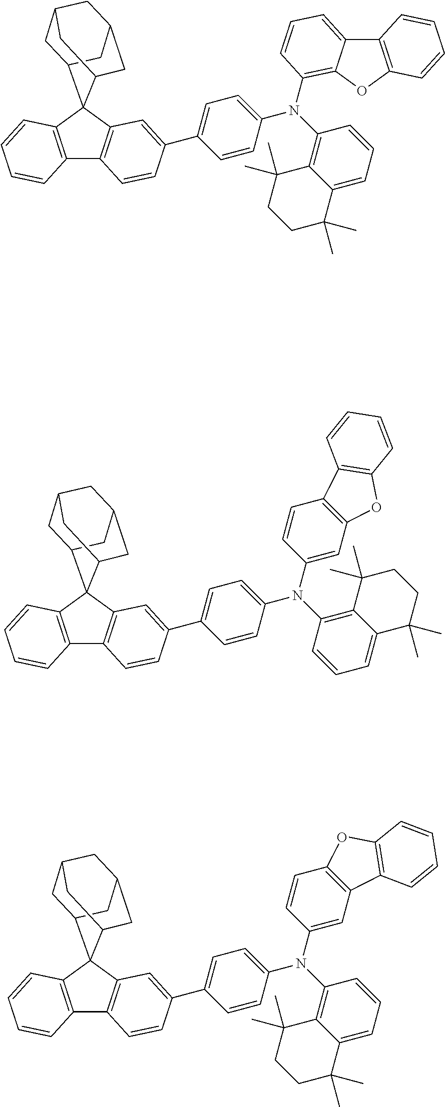 [SMILES: CC1(C)CCC(C)(C)c2c(N(c3ccc(-c4ccc5c(c4)C4(c6ccccc6-5)C5CC6CC(C5)CC4C6)cc3)c3cccc4c3oc3ccccc34)cccc21 ; CC1(C)CCC(C)(C)c2c(N(c3ccc(-c4ccc5c(c4)C4(c6ccccc6-5)C5CC6CC(C5)CC4C6)cc3)c3ccc4c(c3)oc3ccccc34)cccc21 ; CC1(C)CCC(C)(C)c2c(N(c3ccc(-c4ccc5c(c4)C4(c6ccccc6-5)C5CC6CC(C5)CC4C6)cc3)c3ccc4oc5ccccc5c4c3)cccc21]

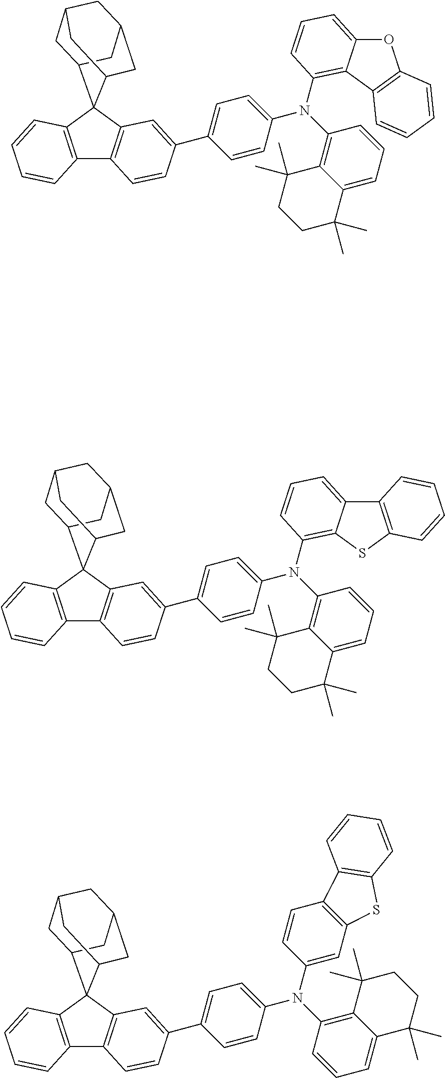 [SMILES: CC1(C)CCC(C)(C)c2c(N(c3ccc(-c4ccc5c(c4)C4(c6ccccc6-5)C5CC6CC(C5)CC4C6)cc3)c3cccc4oc5ccccc5c34)cccc21 ; CC1(C)CCC(C)(C)c2c(N(c3ccc(-c4ccc5c(c4)C4(c6ccccc6-5)C5CC6CC(C5)CC4C6)cc3)c3cccc4c3sc3ccccc34)cccc21 ; CC1(C)CCC(C)(C)c2c(N(c3ccc(-c4ccc5c(c4)C4(c6ccccc6-5)C5CC6CC(C5)CC4C6)cc3)c3ccc4c(c3)sc3ccccc34)cccc21]

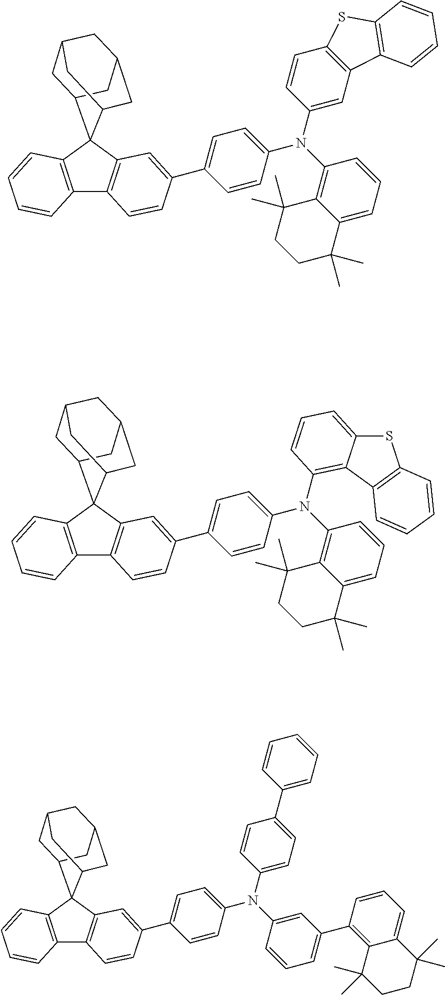 [SMILES: CC1(C)CCC(C)(C)c2c(N(c3ccc(-c4ccc5c(c4)C4(c6ccccc6-5)C5CC6CC(C5)CC4C6)cc3)c3ccc4sc5ccccc5c4c3)cccc21 ; CC1(C)CCC(C)(C)c2c(N(c3ccc(-c4ccc5c(c4)C4(c6ccccc6-5)C5CC6CC(C5)CC4C6)cc3)c3cccc4sc5ccccc5c34)cccc21 ; CC1(C)CCC(C)(C)c2c(-c3cccc(N(c4ccc(-c5ccccc5)cc4)c4ccc(-c5ccc6c(c5)C5(c7ccccc7-6)C6CC7CC(C6)CC5C7)cc4)c3)cccc21]

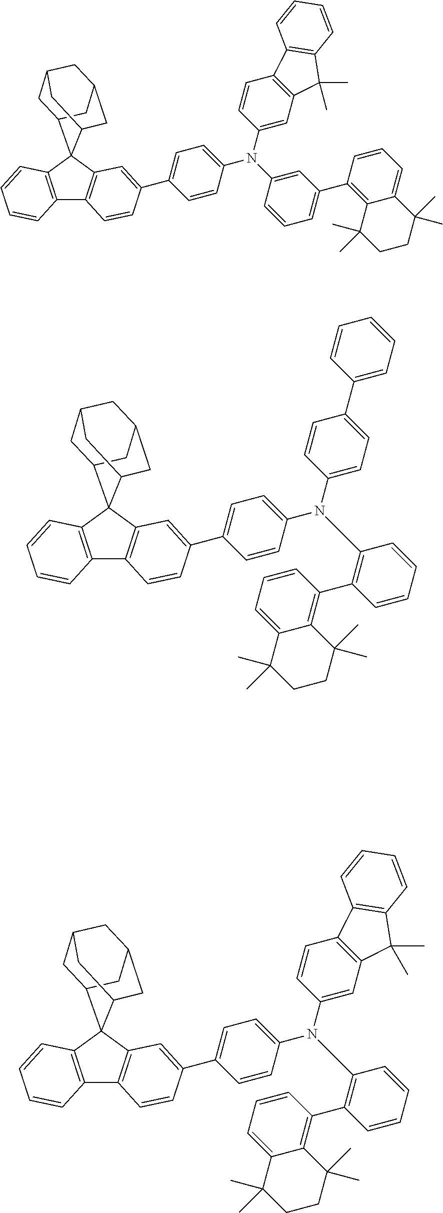 [SMILES: CC1(C)CCC(C)(C)c2c(-c3cccc(N(c4ccc(-c5ccc6c(c5)C5(c7ccccc7-6)C6CC7CC(C6)CC5C7)cc4)c4ccc5c(c4)C(C)(C)c4ccccc4-5)c3)cccc21 ; CC1(C)CCC(C)(C)c2c(-c3ccccc3N(c3ccc(-c4ccccc4)cc3)c3ccc(-c4ccc5c(c4)C4(c6ccccc6-5)C5CC6CC(C5)CC4C6)cc3)cccc21 ; CC1(C)CCC(C)(C)c2c(-c3ccccc3N(c3ccc(-c4ccc5c(c4)C4(c6ccccc6-5)C5CC6CC(C5)CC4C6)cc3)c3ccc4c(c3)C(C)(C)c3ccccc3-4)cccc21]

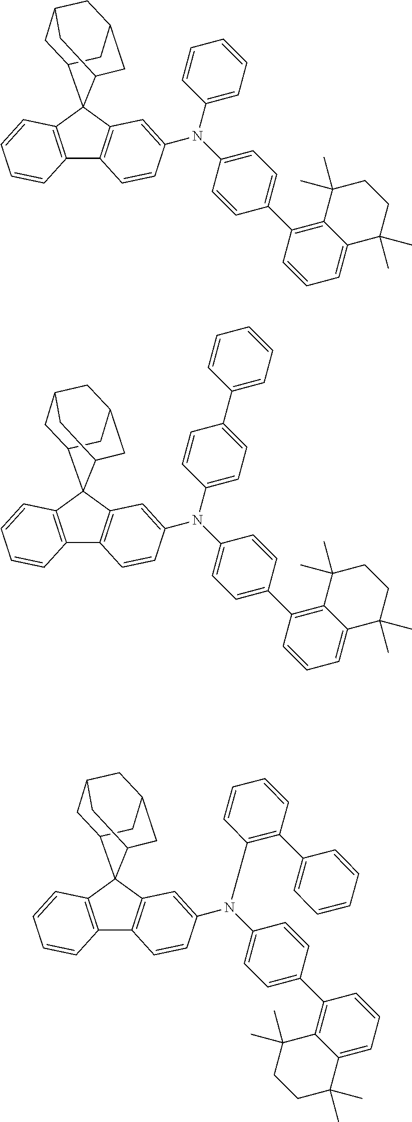 [SMILES: CC1(C)CCC(C)(C)c2c(-c3ccc(N(c4ccccc4)c4ccc5c(c4)C4(c6ccccc6-5)C5CC6CC(C5)CC4C6)cc3)cccc21 ; CC1(C)CCC(C)(C)c2c(-c3ccc(N(c4ccc(-c5ccccc5)cc4)c4ccc5c(c4)C4(c6ccccc6-5)C5CC6CC(C5)CC4C6)cc3)cccc21 ; CC1(C)CCC(C)(C)c2c(-c3ccc(N(c4ccc5c(c4)C4(c6ccccc6-5)C5CC6CC(C5)CC4C6)c4ccccc4-c4ccccc4)cc3)cccc21]

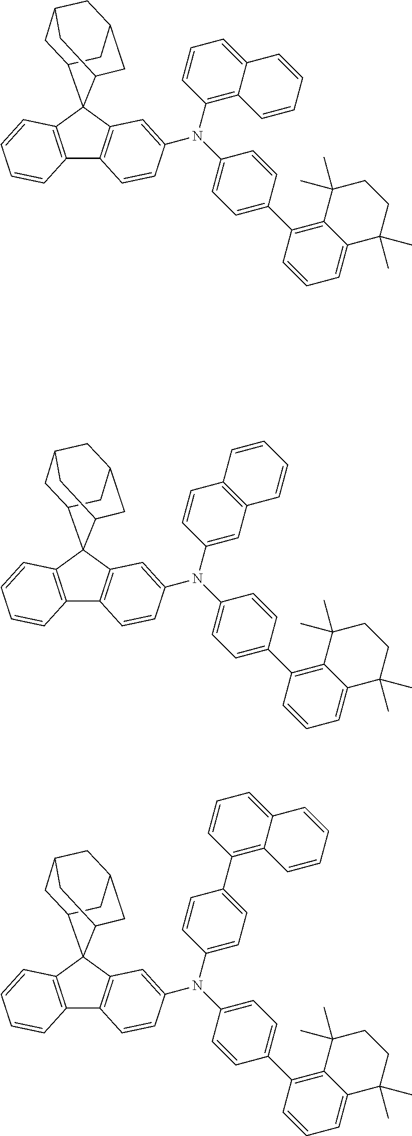 [SMILES: CC1(C)CCC(C)(C)c2c(-c3ccc(N(c4ccc5c(c4)C4(c6ccccc6-5)C5CC6CC(C5)CC4C6)c4cccc5ccccc45)cc3)cccc21 ; CC1(C)CCC(C)(C)c2c(-c3ccc(N(c4ccc5c(c4)C4(c6ccccc6-5)C5CC6CC(C5)CC4C6)c4ccc5ccccc5c4)cc3)cccc21 ; CC1(C)CCC(C)(C)c2c(-c3ccc(N(c4ccc(-c5cccc6ccccc56)cc4)c4ccc5c(c4)C4(c6ccccc6-5)C5CC6CC(C5)CC4C6)cc3)cccc21]

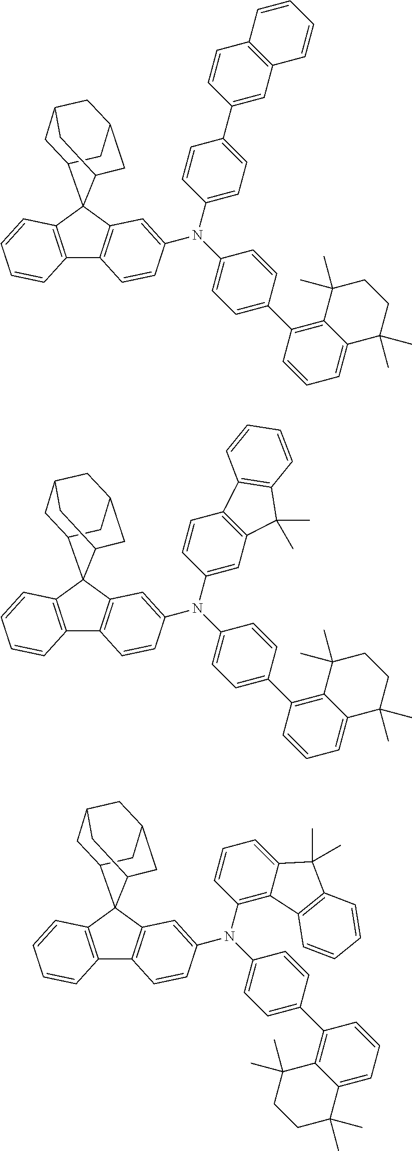 [SMILES: CC1(C)CCC(C)(C)c2c(-c3ccc(N(c4ccc(-c5ccc6ccccc6c5)cc4)c4ccc5c(c4)C4(c6ccccc6-5)C5CC6CC(C5)CC4C6)cc3)cccc21 ; CC1(C)CCC(C)(C)c2c(-c3ccc(N(c4ccc5c(c4)C(C)(C)c4ccccc4-5)c4ccc5c(c4)C4(c6ccccc6-5)C5CC6CC(C5)CC4C6)cc3)cccc21 ; CC1(C)CCC(C)(C)c2c(-c3ccc(N(c4ccc5c(c4)C4(c6ccccc6-5)C5CC6CC(C5)CC4C6)c4cccc5c4-c4ccccc4C5(C)C)cc3)cccc21]

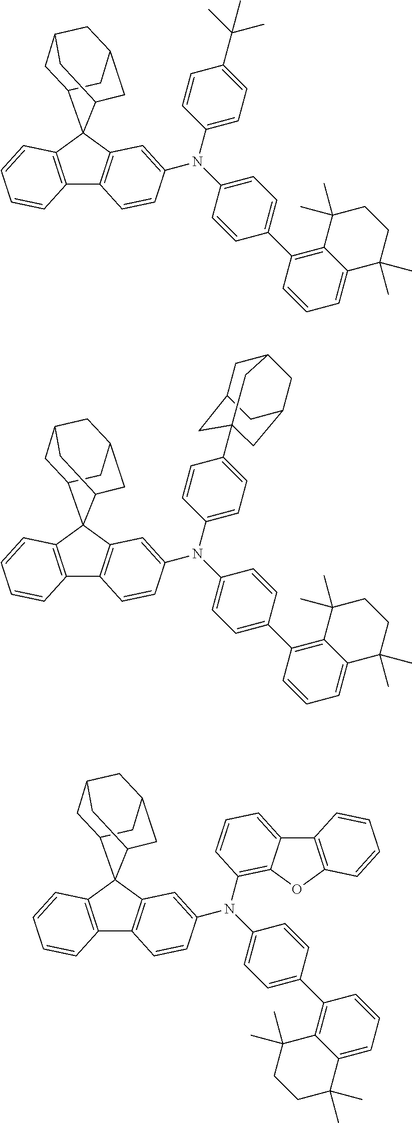 [SMILES: CC(C)(C)c1ccc(N(c2ccc(-c3cccc4c3C(C)(C)CCC4(C)C)cc2)c2ccc3c(c2)C2(c4ccccc4-3)C3CC4CC(C3)CC2C4)cc1 ; CC1(C)CCC(C)(C)c2c(-c3ccc(N(c4ccc(C56CC7CC(CC(C7)C5)C6)cc4)c4ccc5c(c4)C4(c6ccccc6-5)C5CC6CC(C5)CC4C6)cc3)cccc21 ; CC1(C)CCC(C)(C)c2c(-c3ccc(N(c4ccc5c(c4)C4(c6ccccc6-5)C5CC6CC(C5)CC4C6)c4cccc5c4oc4ccccc45)cc3)cccc21]

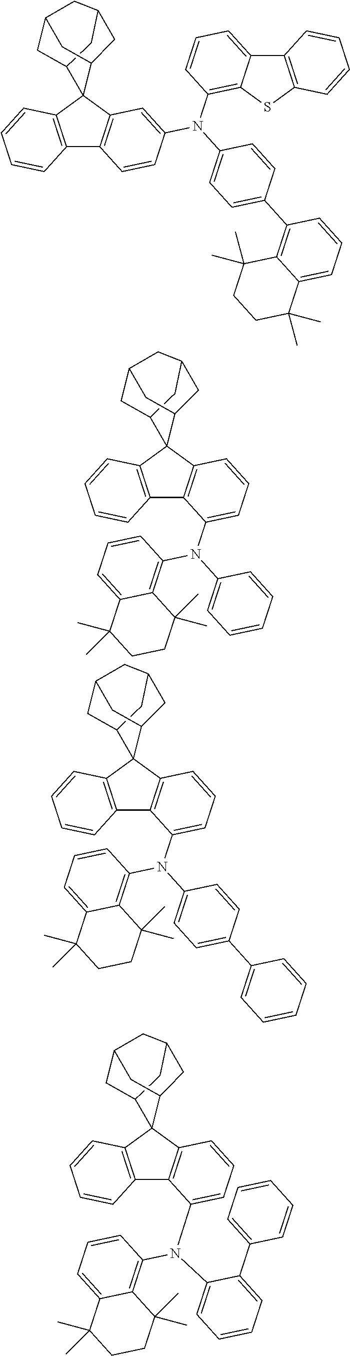

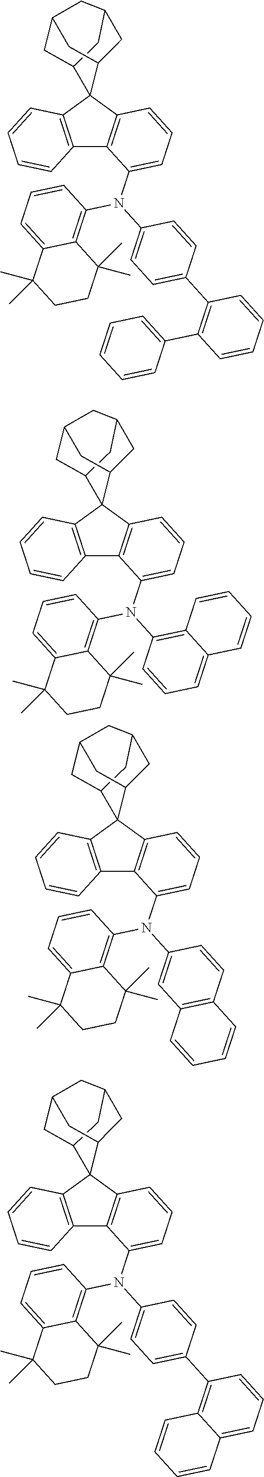

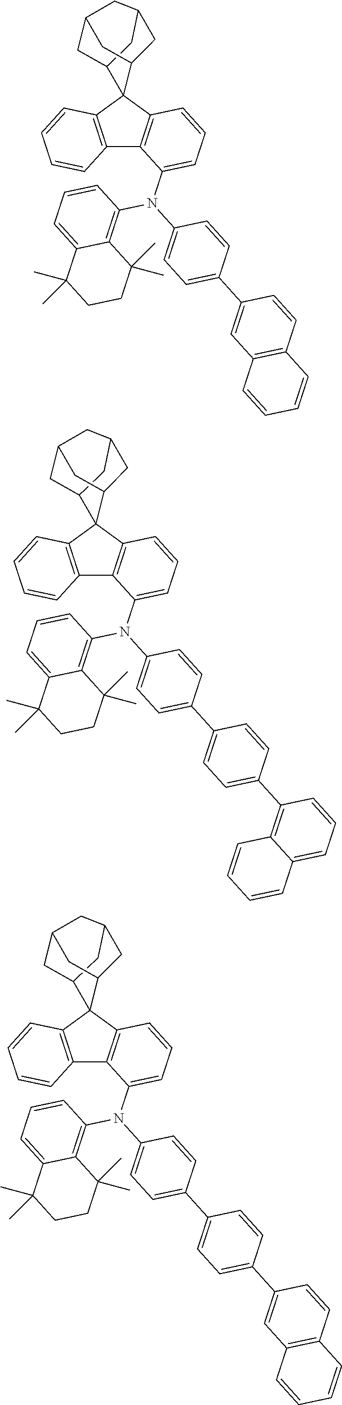 [SMILES: CC1(C)CCC(C)(C)c2c(N(c3ccc(-c4ccc5ccccc5c4)cc3)c3cccc4c3-c3ccccc3C43C4CC5CC(C4)CC3C5)cccc21 ; CC1(C)CCC(C)(C)c2c(N(c3ccc(-c4ccc(-c5cccc6ccccc56)cc4)cc3)c3cccc4c3-c3ccccc3C43C4CC5CC(C4)CC3C5)cccc21 ; CC1(C)CCC(C)(C)c2c(N(c3ccc(-c4ccc(-c5ccc6ccccc6c5)cc4)cc3)c3cccc4c3-c3ccccc3C43C4CC5CC(C4)CC3C5)cccc21]

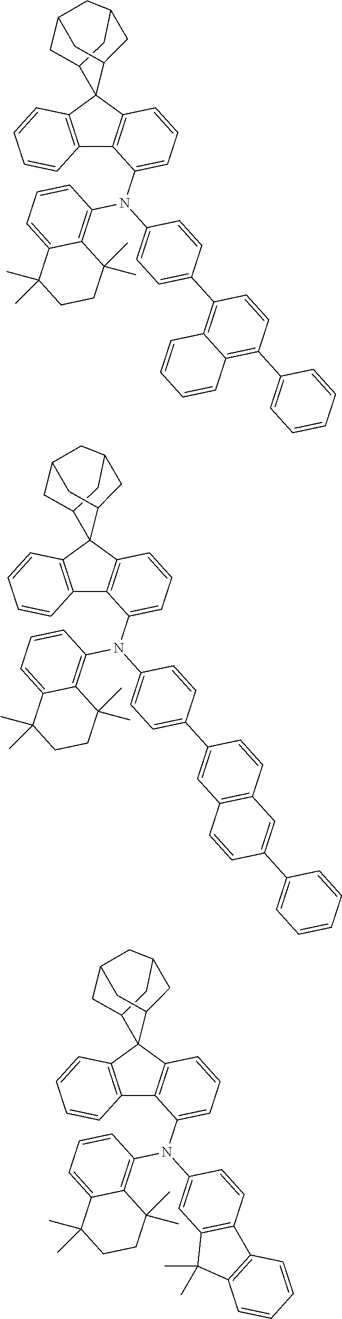 [SMILES: CC1(C)CCC(C)(C)c2c(N(c3ccc(-c4ccc(-c5ccccc5)c5ccccc45)cc3)c3cccc4c3-c3ccccc3C43C4CC5CC(C4)CC3C5)cccc21 ; CC1(C)CCC(C)(C)c2c(N(c3ccc(-c4ccc5cc(-c6ccccc6)ccc5c4)cc3)c3cccc4c3-c3ccccc3C43C4CC5CC(C4)CC3C5)cccc21 ; CC1(C)CCC(C)(C)c2c(N(c3ccc4c(c3)C(C)(C)c3ccccc3-4)c3cccc4c3-c3ccccc3C43C4CC5CC(C4)CC3C5)cccc21]

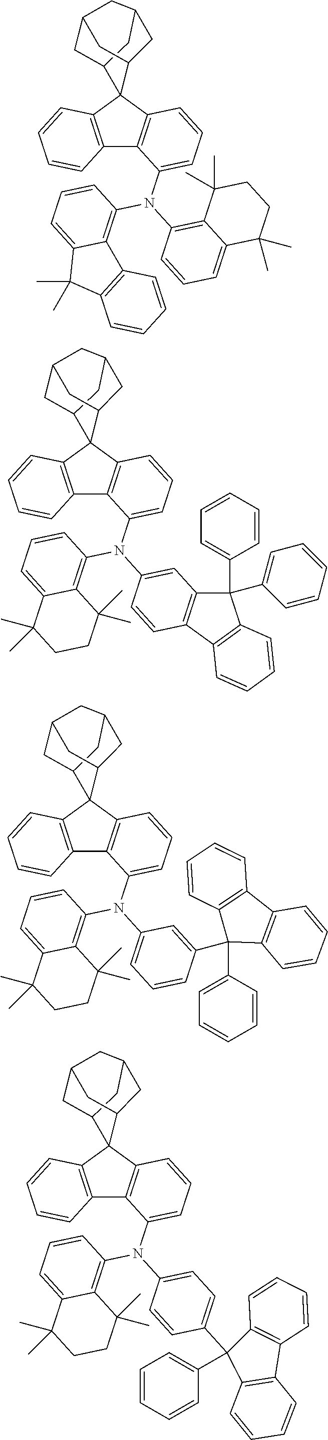

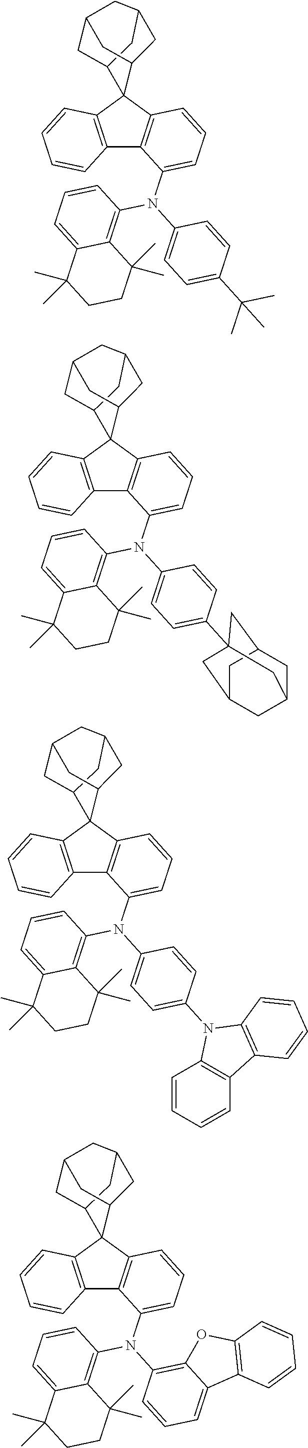

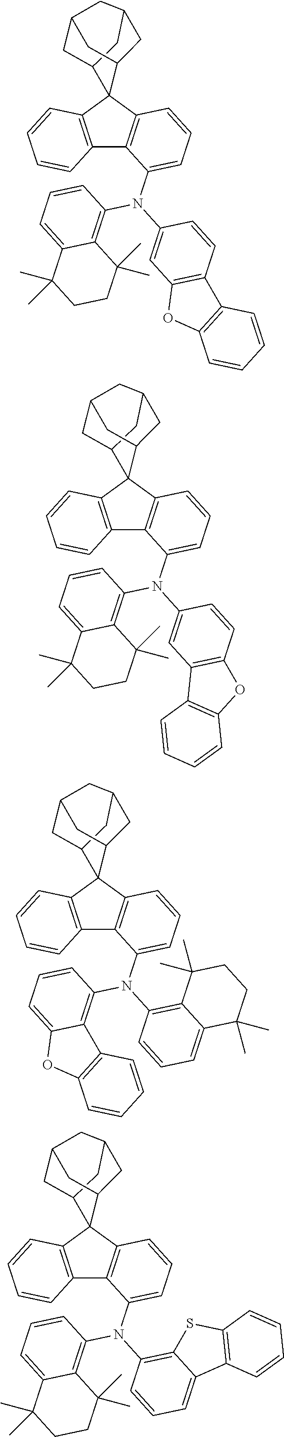

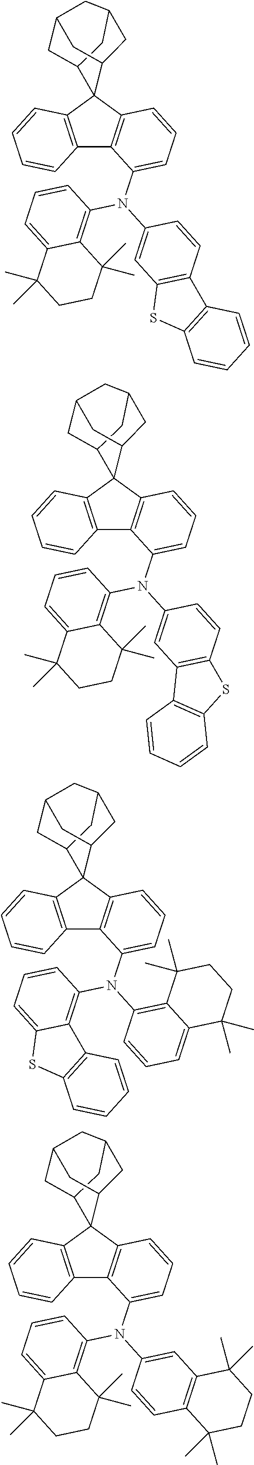

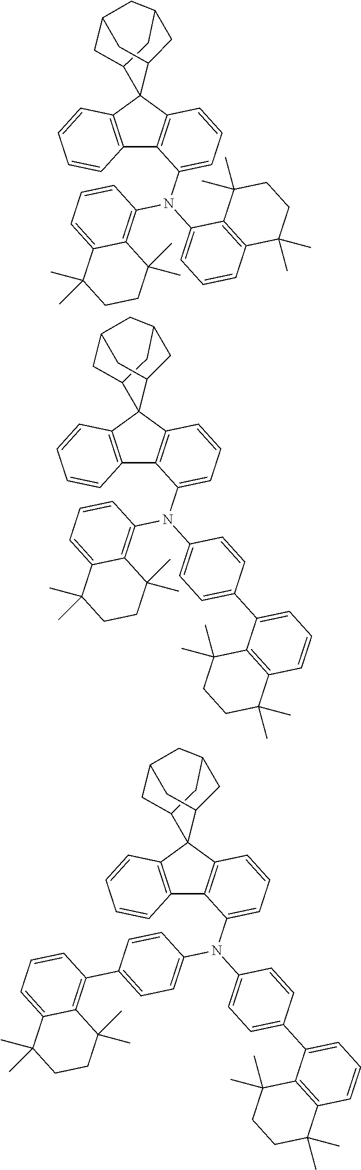

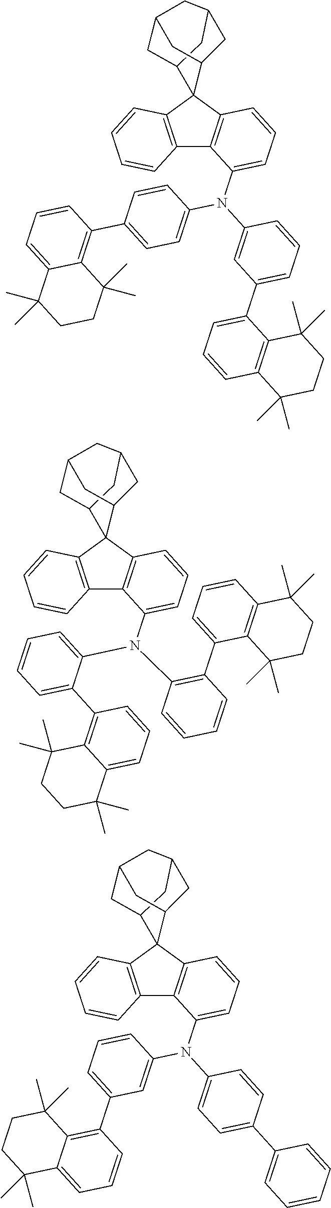

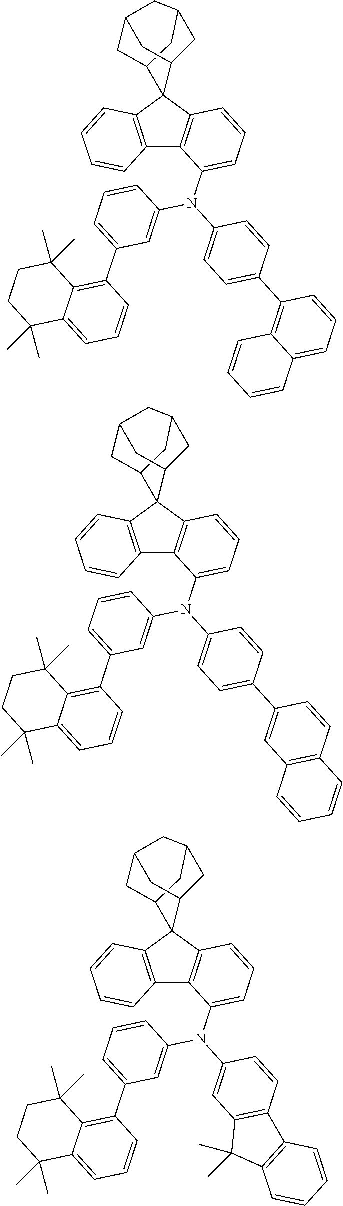 [SMILES: CC1(C)CCC(C)(C)c2c(-c3cccc(N(c4ccc(-c5cccc6ccccc56)cc4)c4cccc5c4-c4ccccc4C54C5CC6CC(C5)CC4C6)c3)cccc21 ; CC1(C)CCC(C)(C)c2c(-c3cccc(N(c4ccc(-c5ccc6ccccc6c5)cc4)c4cccc5c4-c4ccccc4C54C5CC6CC(C5)CC4C6)c3)cccc21 ; CC1(C)CCC(C)(C)c2c(-c3cccc(N(c4ccc5c(c4)C(C)(C)c4ccccc4-5)c4cccc5c4-c4ccccc4C54C5CC6CC(C5)CC4C6)c3)cccc21]

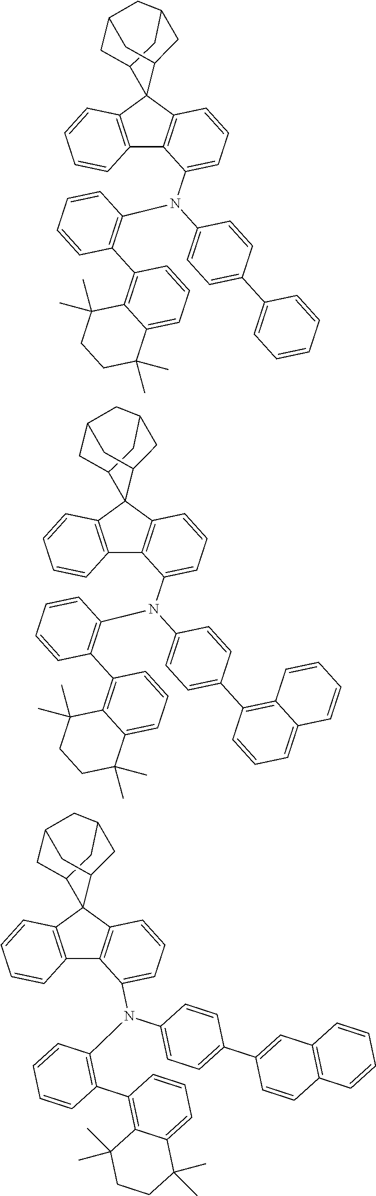

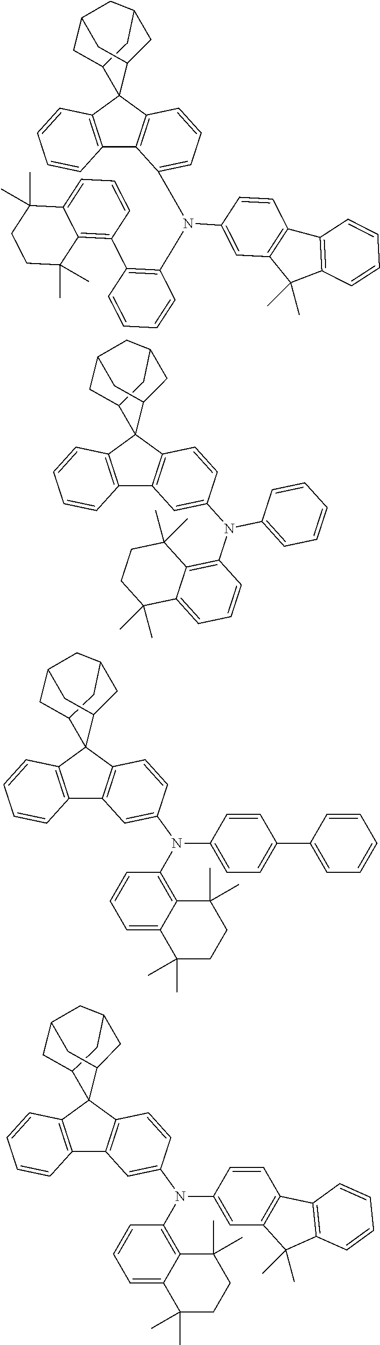

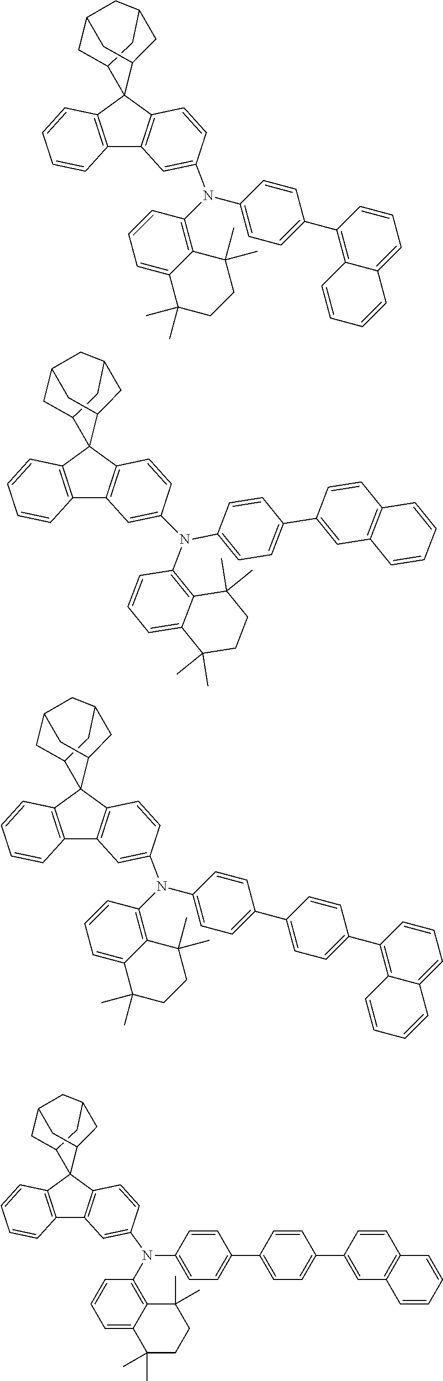 [SMILES: CC1(C)CCC(C)(C)c2c(N(c3ccc(-c4cccc5ccccc45)cc3)c3ccc4c(c3)-c3ccccc3C43C4CC5CC(C4)CC3C5)cccc21 ; CC1(C)CCC(C)(C)c2c(N(c3ccc(-c4ccc5ccccc5c4)cc3)c3ccc4c(c3)-c3ccccc3C43C4CC5CC(C4)CC3C5)cccc21 ; CC1(C)CCC(C)(C)c2c(N(c3ccc(-c4ccc(-c5cccc6ccccc56)cc4)cc3)c3ccc4c(c3)-c3ccccc3C43C4CC5CC(C4)CC3C5)cccc21 ; CC1(C)CCC(C)(C)c2c(N(c3ccc(-c4ccc(-c5ccc6ccccc6c5)cc4)cc3)c3ccc4c(c3)-c3ccccc3C43C4CC5CC(C4)CC3C5)cccc21]

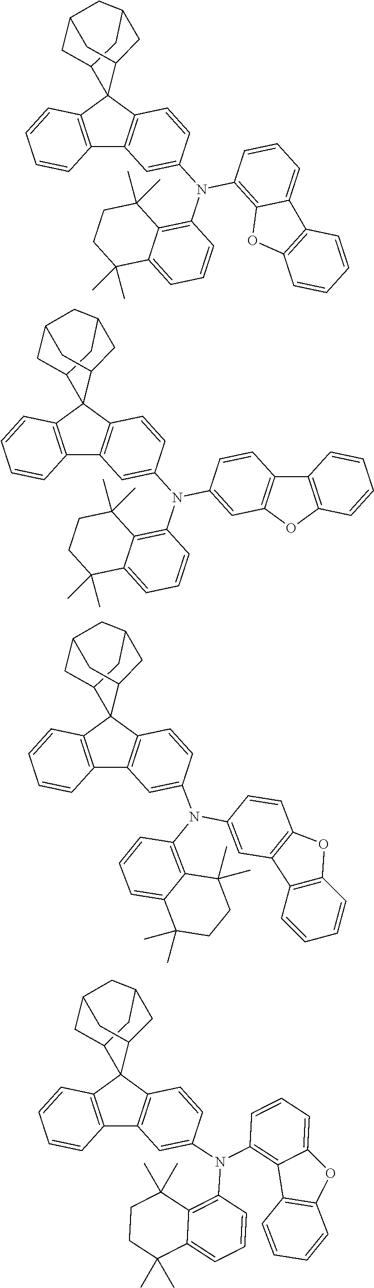

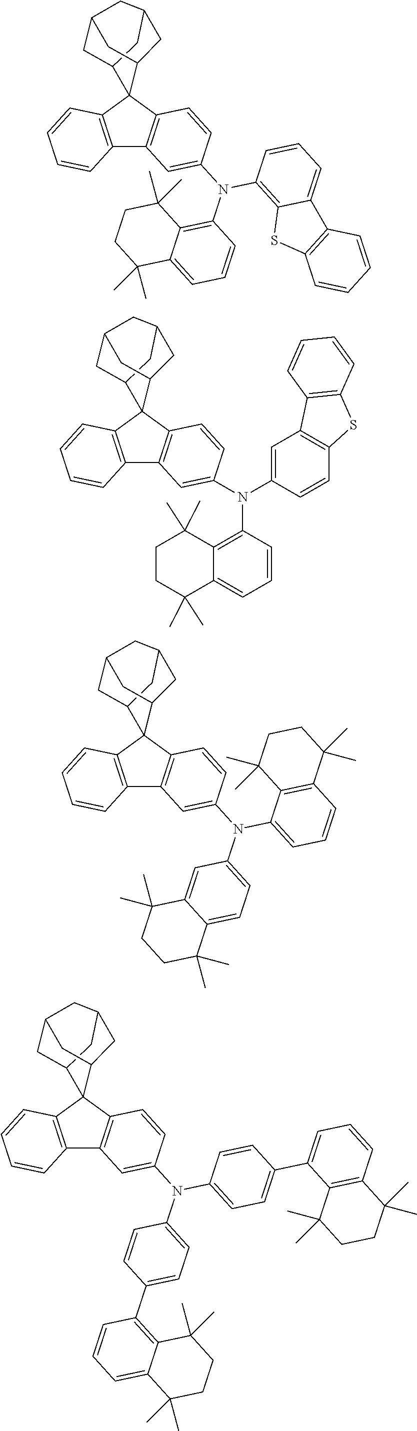

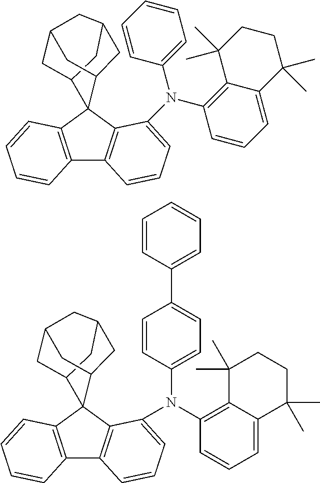

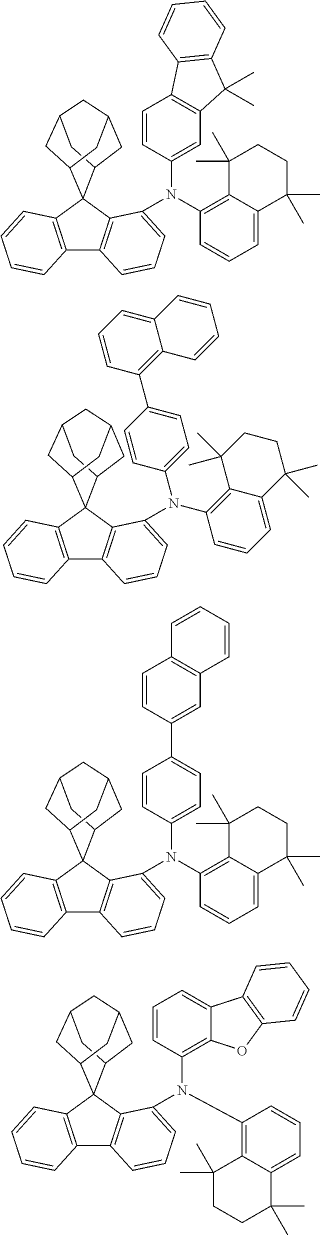

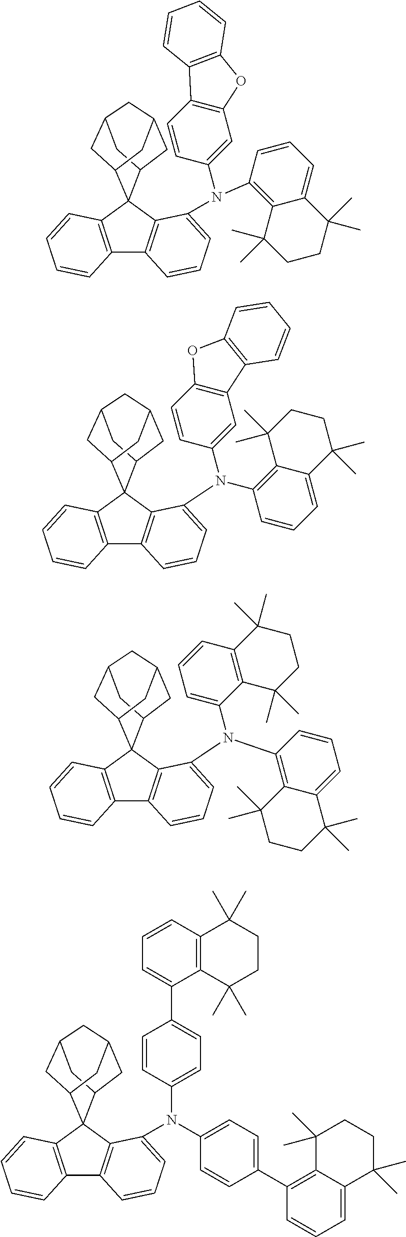

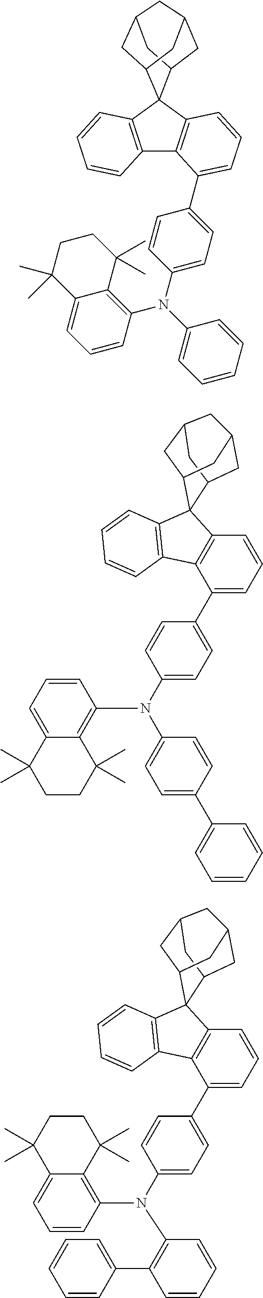

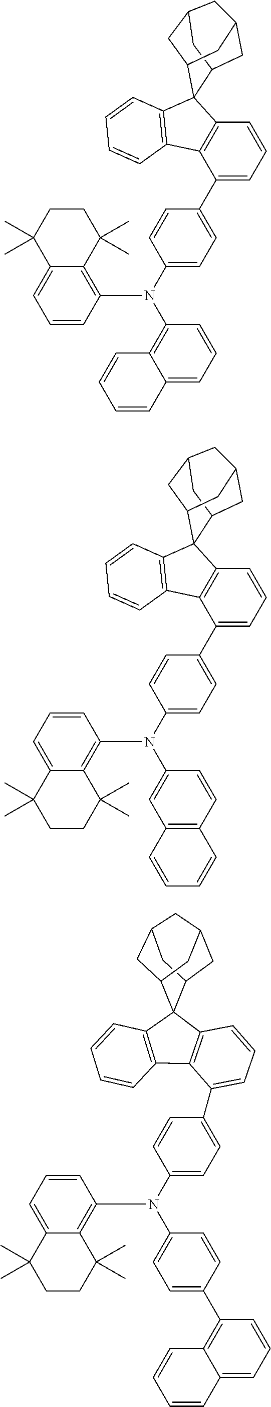

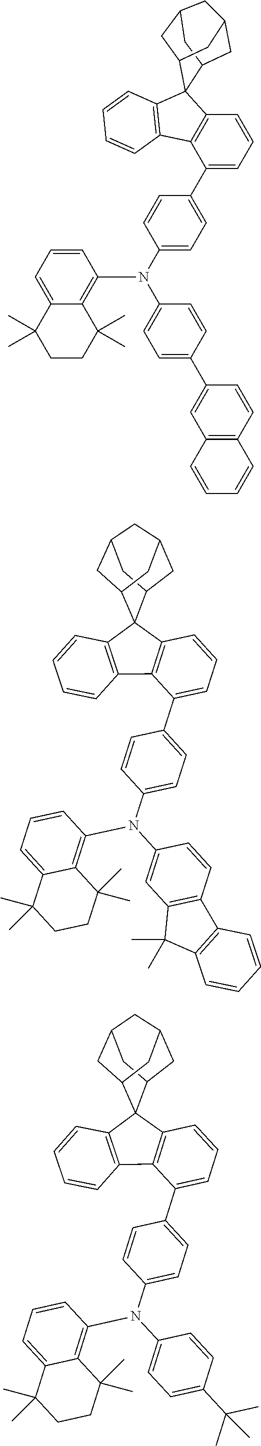 [SMILES: CC1(C)CCC(C)(C)c2c(N(c3ccc(-c4ccc5ccccc5c4)cc3)c3ccc(-c4cccc5c4-c4ccccc4C54C5CC6CC(C5)CC4C6)cc3)cccc21 ; CC1(C)CCC(C)(C)c2c(N(c3ccc(-c4cccc5c4-c4ccccc4C54C5CC6CC(C5)CC4C6)cc3)c3ccc4c(c3)C(C)(C)c3ccccc3-4)cccc21 ; CC(C)(C)c1ccc(N(c2ccc(-c3cccc4c3-c3ccccc3C43C4CC5CC(C4)CC3C5)cc2)c2cccc3c2C(C)(C)CCC3(C)C)cc1]

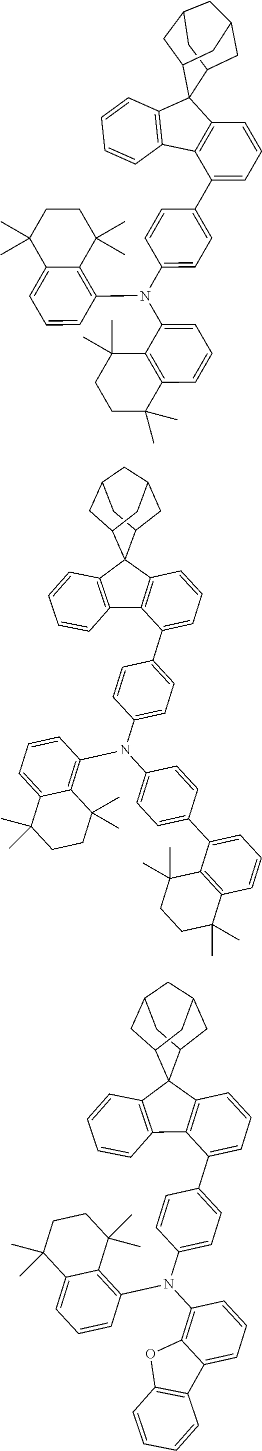

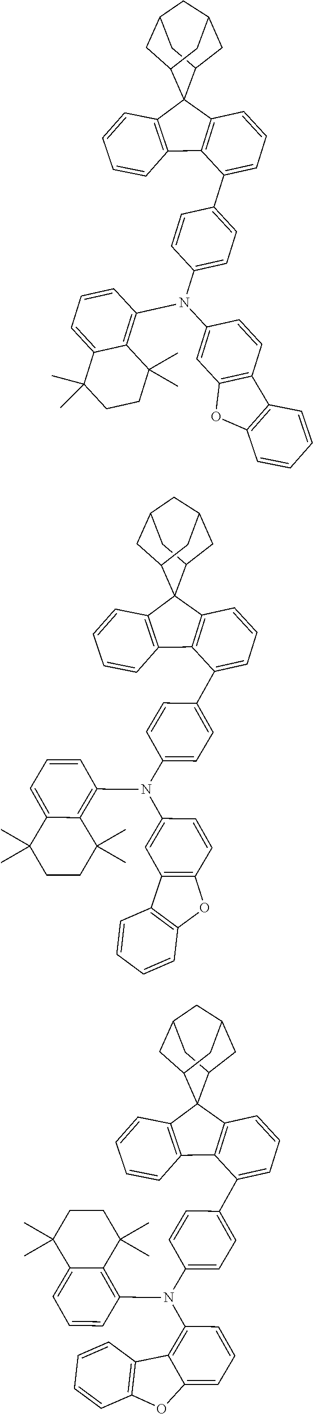 [SMILES: CC1(C)CCC(C)(C)c2c(N(c3ccc(-c4cccc5c4-c4ccccc4C54C5CC6CC(C5)CC4C6)cc3)c3ccc4c(c3)oc3ccccc34)cccc21 ; CC1(C)CCC(C)(C)c2c(N(c3ccc(-c4cccc5c4-c4ccccc4C54C5CC6CC(C5)CC4C6)cc3)c3ccc4oc5ccccc5c4c3)cccc21 ; CC1(C)CCC(C)(C)c2c(N(c3ccc(-c4cccc5c4-c4ccccc4C54C5CC6CC(C5)CC4C6)cc3)c3cccc4oc5ccccc5c34)cccc21]

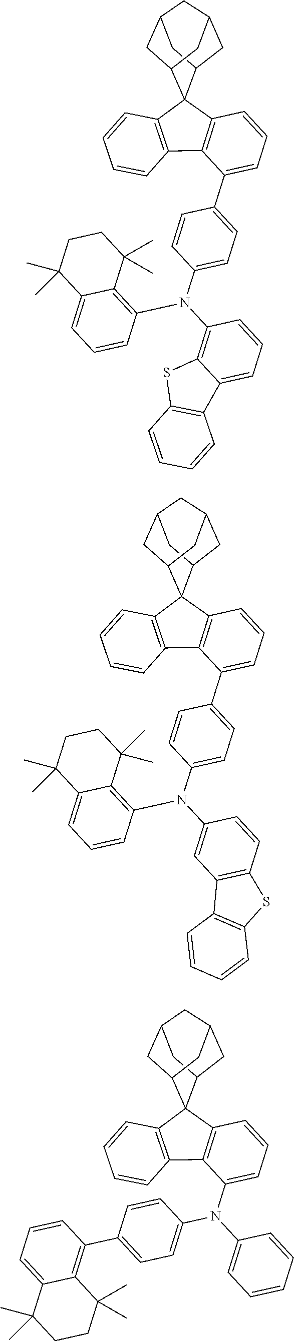

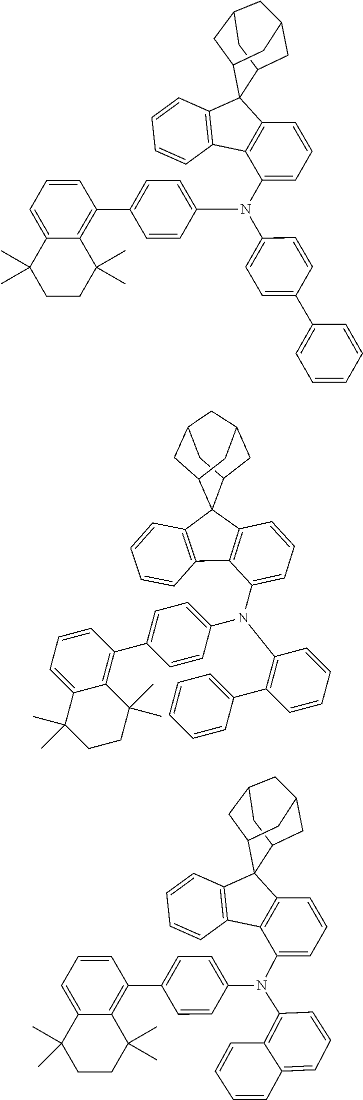 [SMILES: CC1(C)CCC(C)(C)c2c(-c3ccc(N(c4ccc(-c5ccccc5)cc4)c4cccc5c4-c4ccccc4C54C5CC6CC(C5)CC4C6)cc3)cccc21 ; CC1(C)CCC(C)(C)c2c(-c3ccc(N(c4ccccc4-c4ccccc4)c4cccc5c4-c4ccccc4C54C5CC6CC(C5)CC4C6)cc3)cccc21 ; CC1(C)CCC(C)(C)c2c(-c3ccc(N(c4cccc5c4-c4ccccc4C54C5CC6CC(C5)CC4C6)c4cccc5ccccc45)cc3)cccc21]

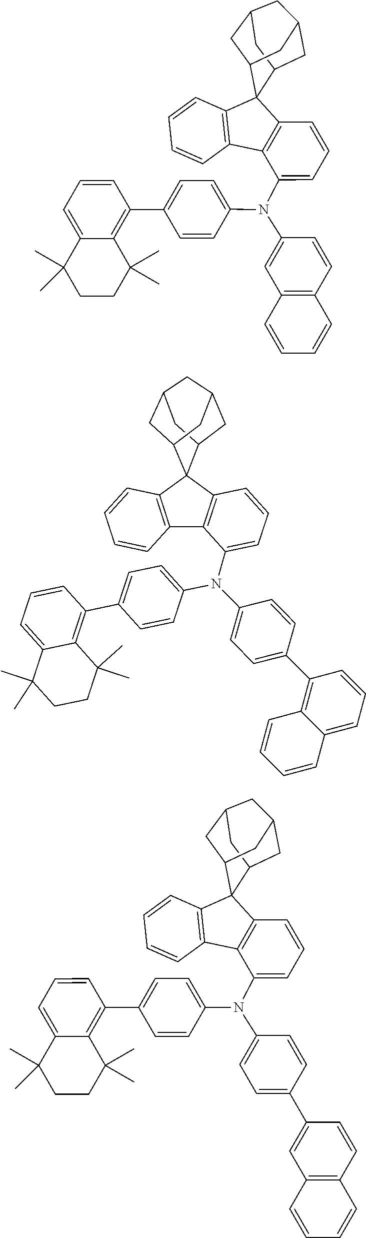

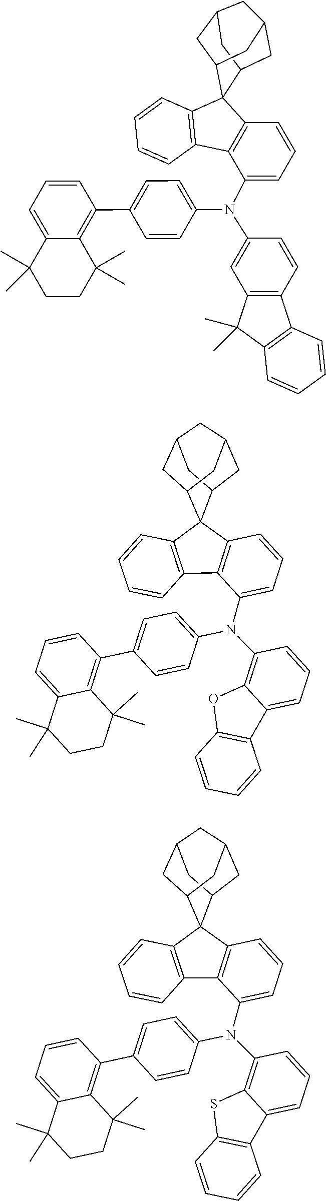

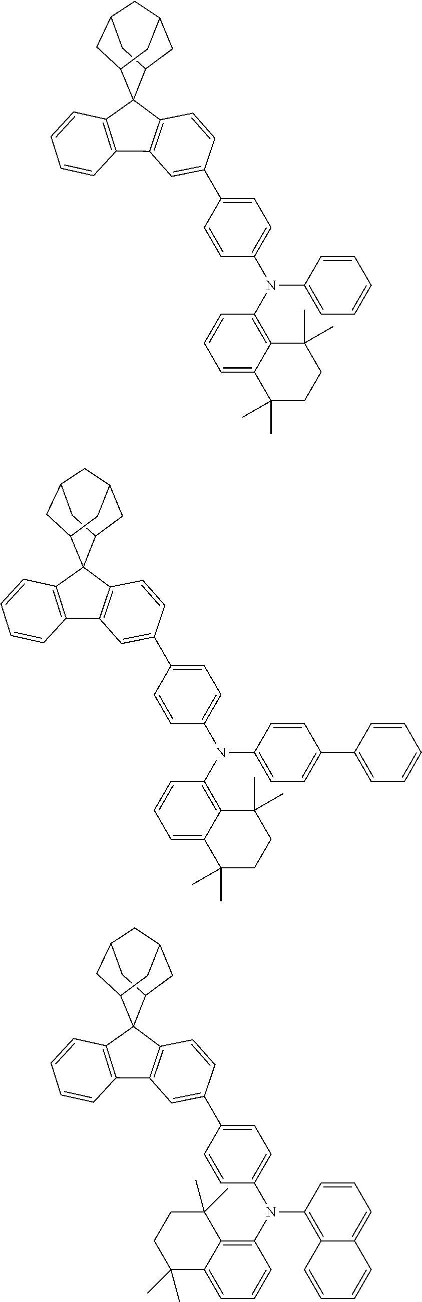 [SMILES: CC1(C)CCC(C)(C)c2c(N(c3ccccc3)c3ccc(-c4ccc5c(c4)-c4ccccc4C54C5CC6CC(C5)CC4C6)cc3)cccc21 ; CC1(C)CCC(C)(C)c2c(N(c3ccc(-c4ccccc4)cc3)c3ccc(-c4ccc5c(c4)-c4ccccc4C54C5CC6CC(C5)CC4C6)cc3)cccc21 ; CC1(C)CCC(C)(C)c2c(N(c3ccc(-c4ccc5c(c4)-c4ccccc4C54C5CC6CC(C5)CC4C6)cc3)c3cccc4ccccc34)cccc21]

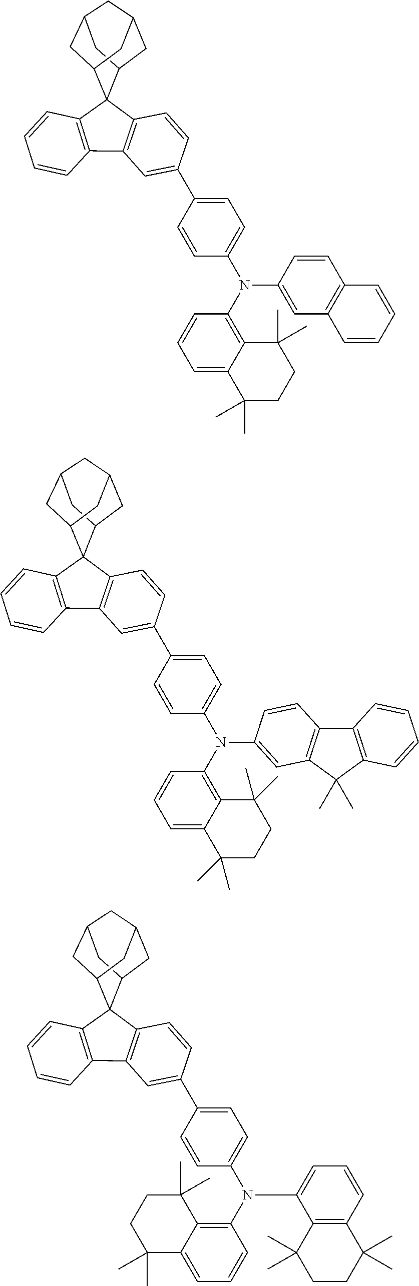 [SMILES: CC1(C)CCC(C)(C)c2c(N(c3ccc(-c4ccc5c(c4)-c4ccccc4C54C5CC6CC(C5)CC4C6)cc3)c3ccc4ccccc4c3)cccc21 ; CC1(C)CCC(C)(C)c2c(N(c3ccc(-c4ccc5c(c4)-c4ccccc4C54C5CC6CC(C5)CC4C6)cc3)c3ccc4c(c3)C(C)(C)c3ccccc3-4)cccc21 ; CC1(C)CCC(C)(C)c2c(N(c3ccc(-c4ccc5c(c4)-c4ccccc4C54C5CC6CC(C5)CC4C6)cc3)c3cccc4c3C(C)(C)CCC4(C)C)cccc21]

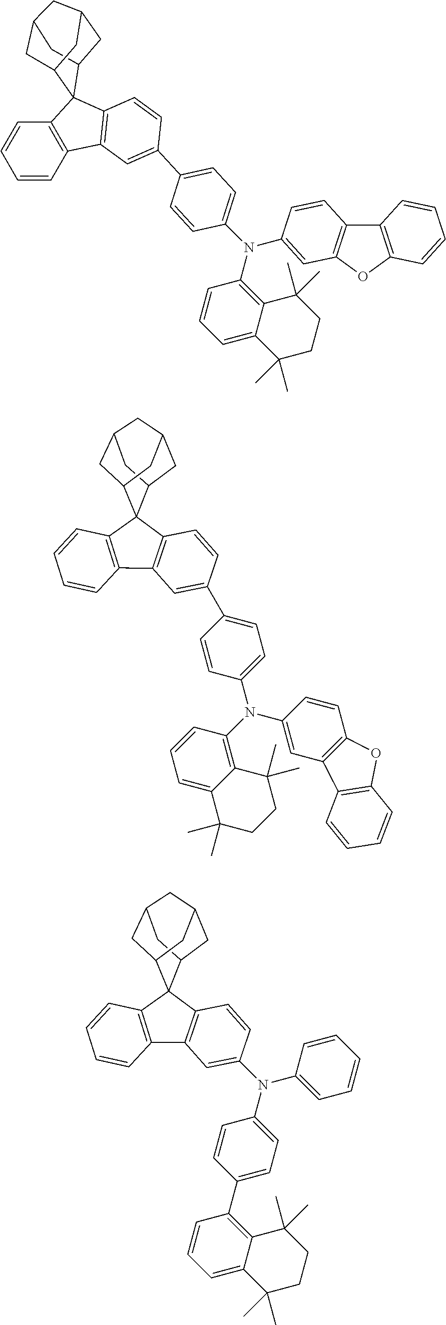 [SMILES: CC1(C)CCC(C)(C)c2c(N(c3ccc(-c4ccc5c(c4)-c4ccccc4C54C5CC6CC(C5)CC4C6)cc3)c3ccc4c(c3)oc3ccccc34)cccc21 ; CC1(C)CCC(C)(C)c2c(N(c3ccc(-c4ccc5c(c4)-c4ccccc4C54C5CC6CC(C5)CC4C6)cc3)c3ccc4oc5ccccc5c4c3)cccc21 ; CC1(C)CCC(C)(C)c2c(-c3ccc(N(c4ccccc4)c4ccc5c(c4)-c4ccccc4C54C5CC6CC(C5)CC4C6)cc3)cccc21]

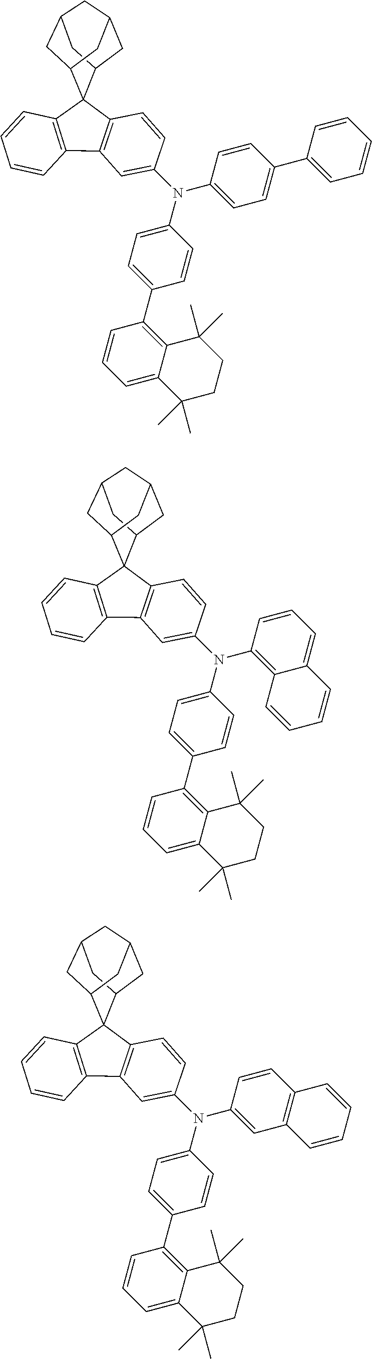 [SMILES: CC1(C)CCC(C)(C)c2c(-c3ccc(N(c4ccc(-c5ccccc5)cc4)c4ccc5c(c4)-c4ccccc4C54C5CC6CC(C5)CC4C6)cc3)cccc21 ; CC1(C)CCC(C)(C)c2c(-c3ccc(N(c4ccc5c(c4)-c4ccccc4C54C5CC6CC(C5)CC4C6)c4cccc5ccccc45)cc3)cccc21 ; CC1(C)CCC(C)(C)c2c(-c3ccc(N(c4ccc5c(c4)-c4ccccc4C54C5CC6CC(C5)CC4C6)c4ccc5ccccc5c4)cc3)cccc21]

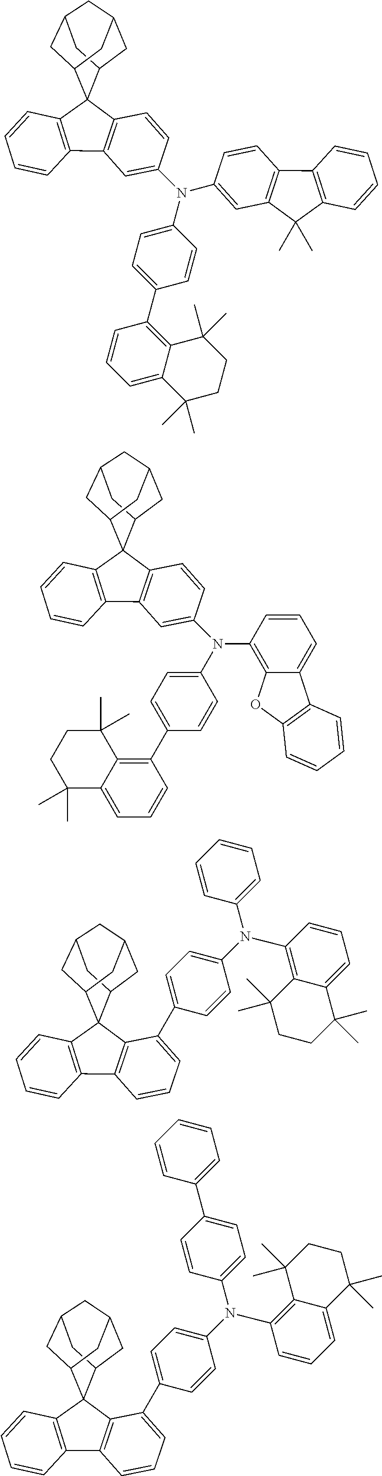

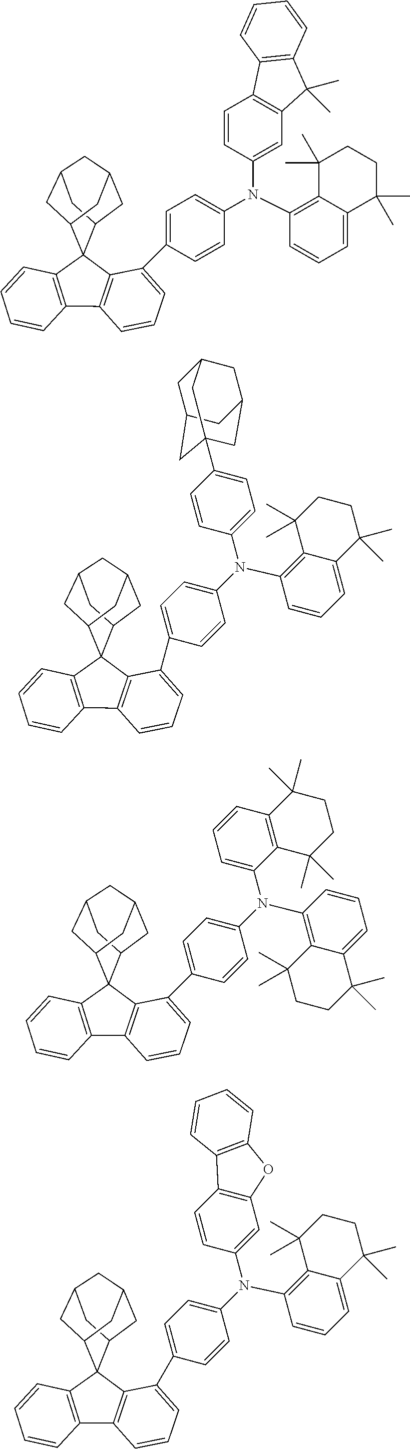 [SMILES: CC1(C)CCC(C)(C)c2c(N(c3ccc(-c4cccc5c4C4(c6ccccc6-5)C5CC6CC(C5)CC4C6)cc3)c3ccc4c(c3)C(C)(C)c3ccccc3-4)cccc21 ; CC1(C)CCC(C)(C)c2c(N(c3ccc(-c4cccc5c4C4(c6ccccc6-5)C5CC6CC(C5)CC4C6)cc3)c3ccc(C45CC6CC(CC(C6)C4)C5)cc3)cccc21 ; CC1(C)CCC(C)(C)c2c(N(c3ccc(-c4cccc5c4C4(c6ccccc6-5)C5CC6CC(C5)CC4C6)cc3)c3cccc4c3C(C)(C)CCC4(C)C)cccc21 ; CC1(C)CCC(C)(C)c2c(N(c3ccc(-c4cccc5c4C4(c6ccccc6-5)C5CC6CC(C5)CC4C6)cc3)c3ccc4c(c3)oc3ccccc34)cccc21]

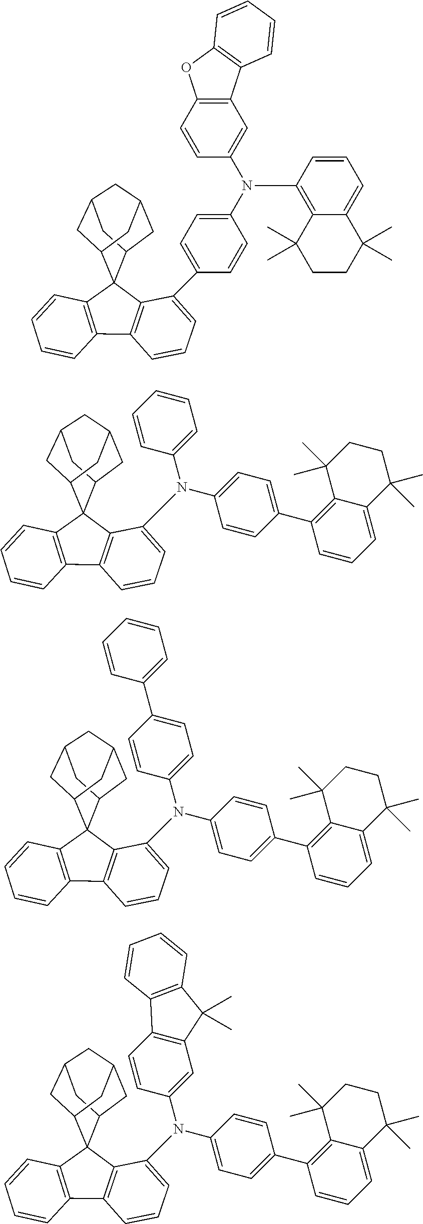

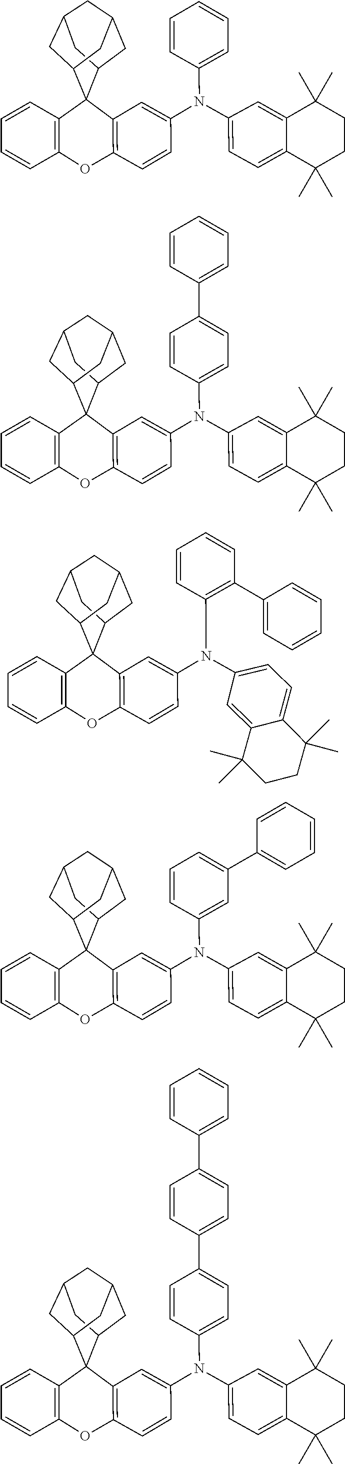

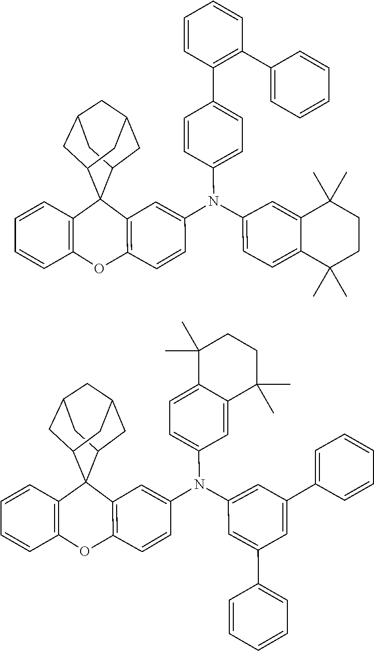 [SMILES: CC1(C)CCC(C)(C)c2cc(N(c3ccc(-c4ccccc4-c4ccccc4)cc3)c3ccc4c(c3)C3(c5ccccc5O4)C4CC5CC(C4)CC3C5)ccc21 ; CC1(C)CCC(C)(C)c2cc(N(c3cc(-c4ccccc4)cc(-c4ccccc4)c3)c3ccc4c(c3)C3(c5ccccc5O4)C4CC5CC(C4)CC3C5)ccc21]

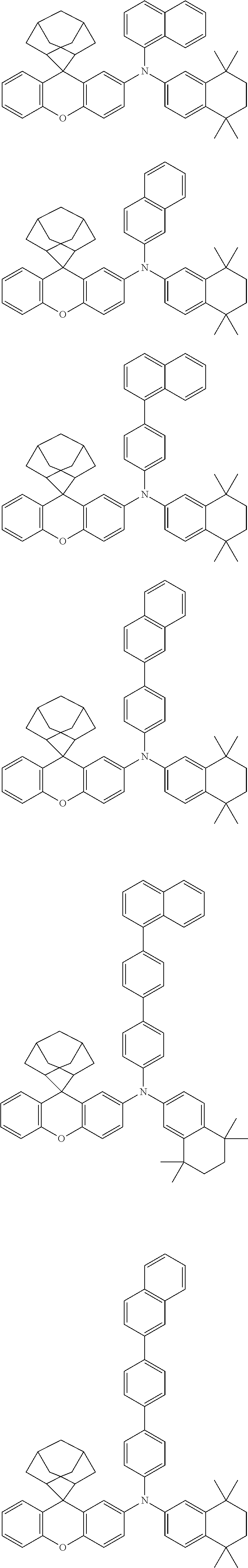

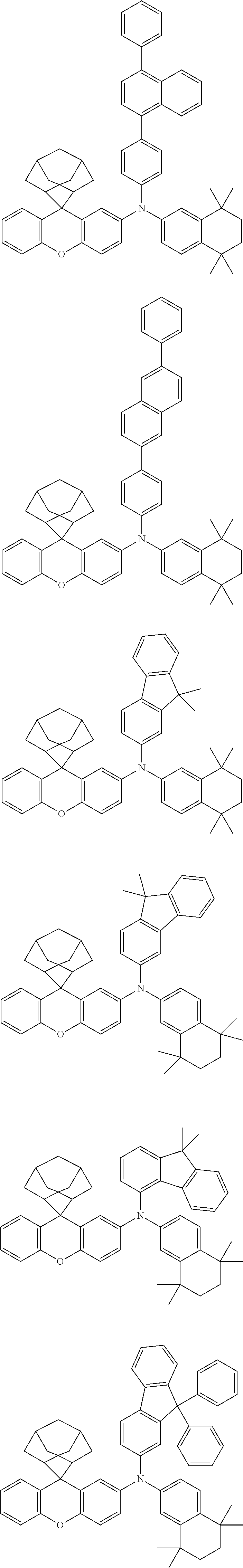

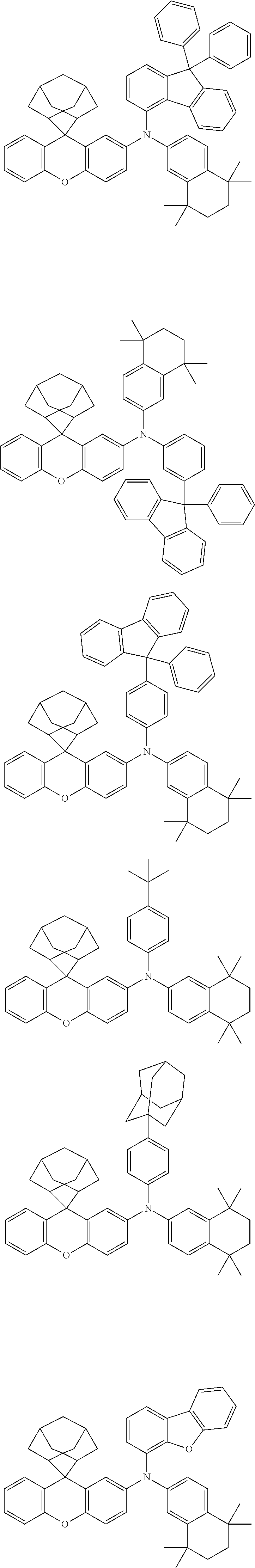

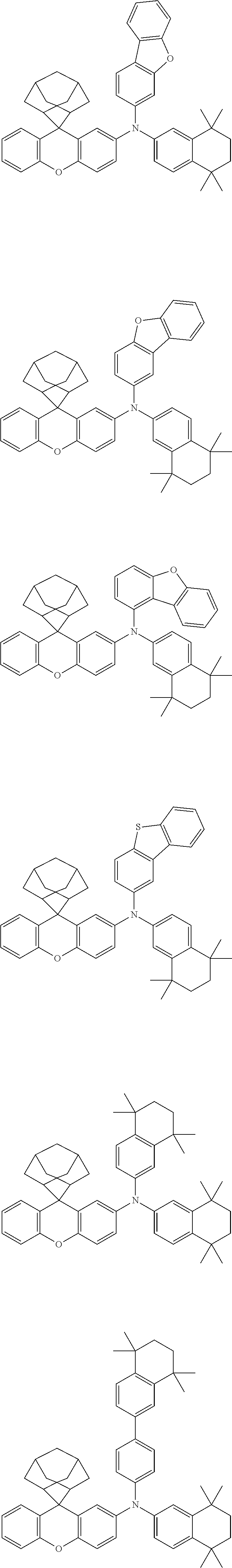

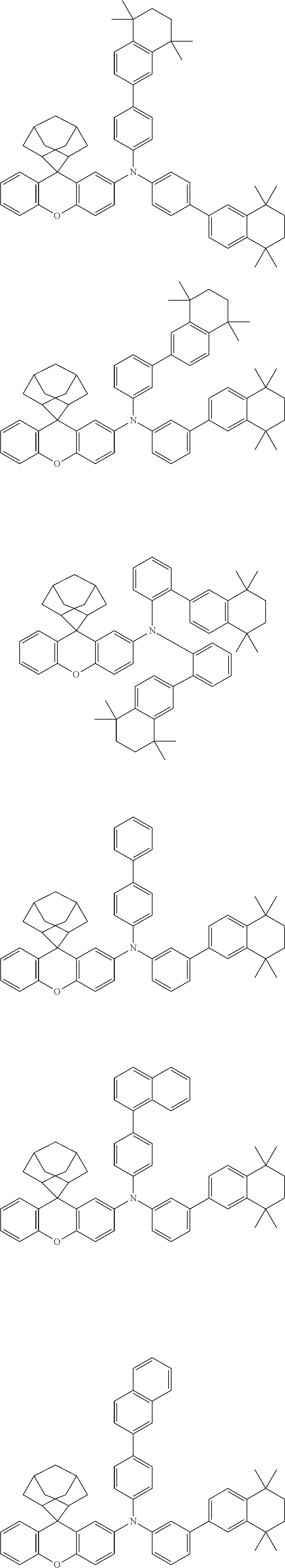

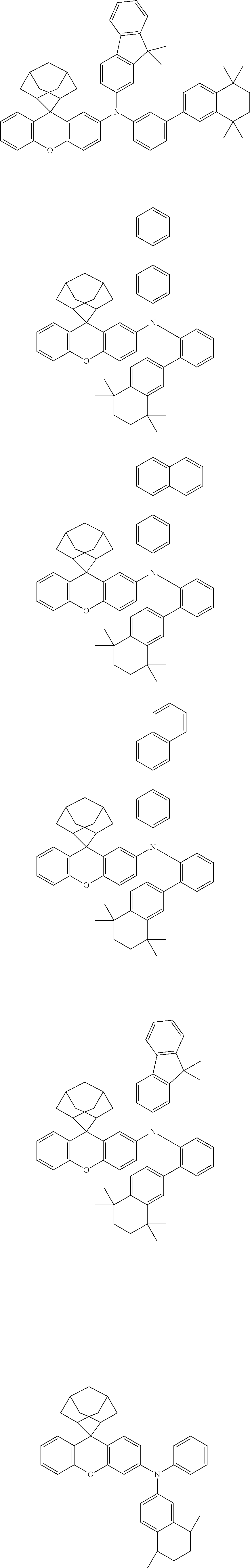

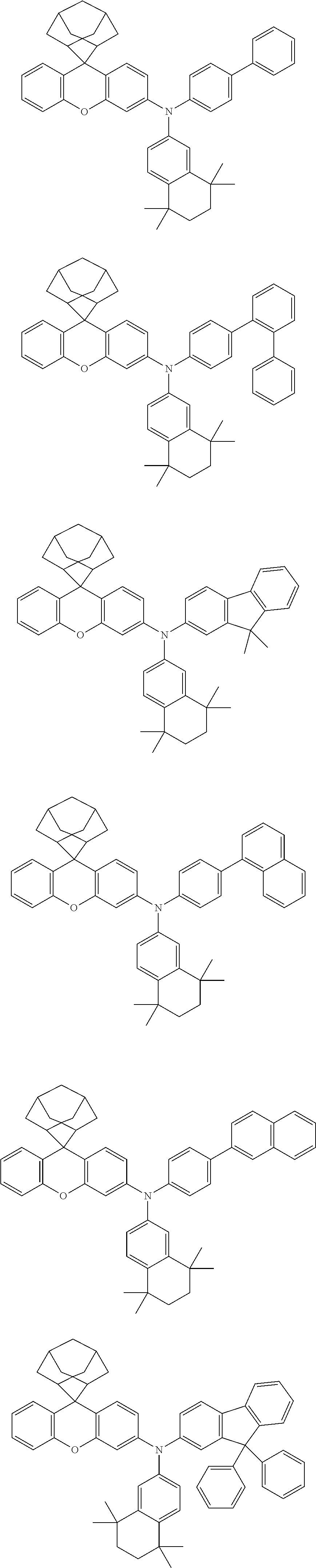

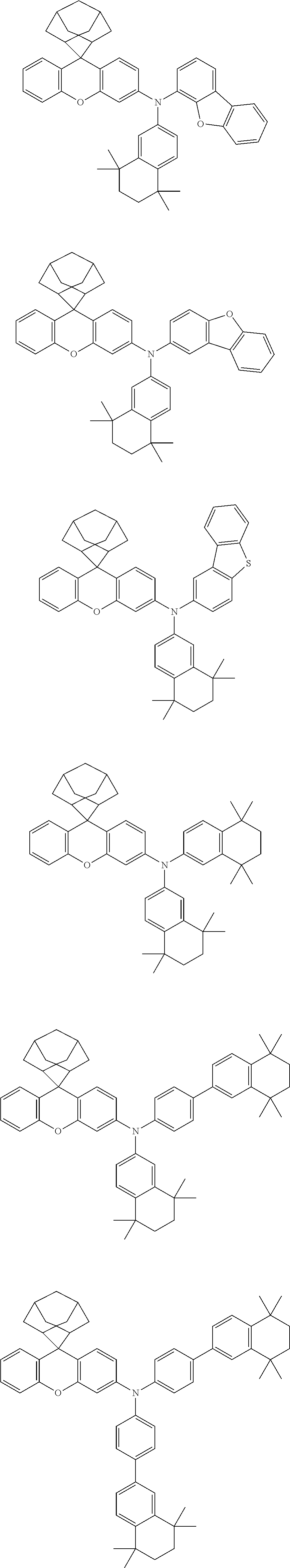

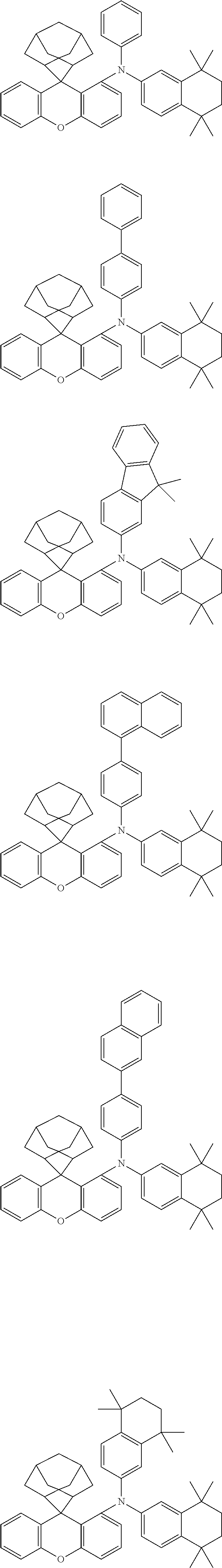

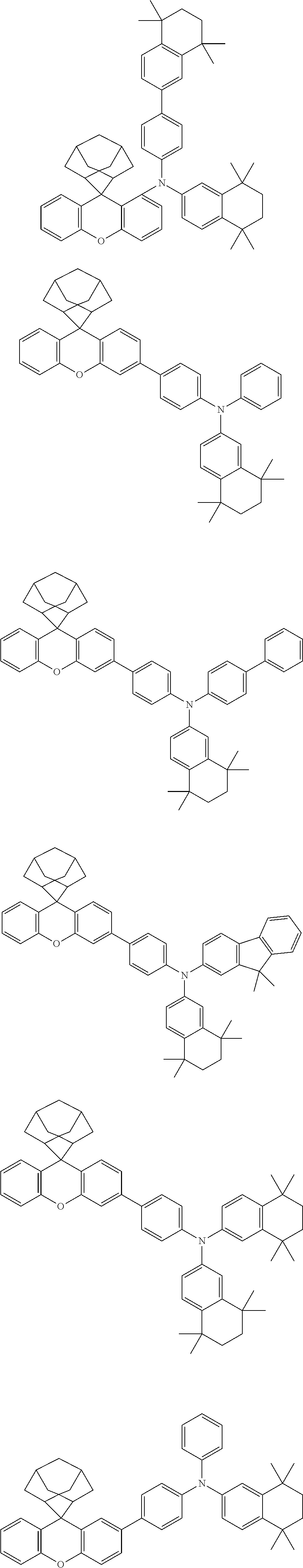

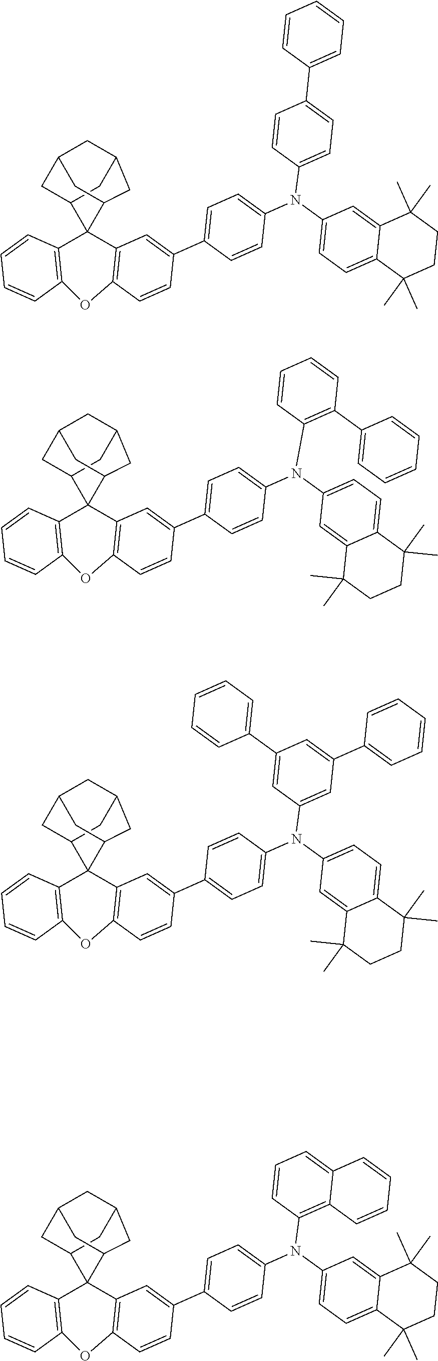 [SMILES: CC1(C)CCC(C)(C)c2cc(N(c3ccc(-c4ccccc4)cc3)c3ccc(-c4ccc5c(c4)C4(c6ccccc6O5)C5CC6CC(C5)CC4C6)cc3)ccc21 ; CC1(C)CCC(C)(C)c2cc(N(c3ccc(-c4ccc5c(c4)C4(c6ccccc6O5)C5CC6CC(C5)CC4C6)cc3)c3ccccc3-c3ccccc3)ccc21 ; CC1(C)CCC(C)(C)c2cc(N(c3ccc(-c4ccc5c(c4)C4(c6ccccc6O5)C5CC6CC(C5)CC4C6)cc3)c3cc(-c4ccccc4)cc(-c4ccccc4)c3)ccc21 ; CC1(C)CCC(C)(C)c2cc(N(c3ccc(-c4ccc5c(c4)C4(c6ccccc6O5)C5CC6CC(C5)CC4C6)cc3)c3cccc4ccccc34)ccc21]

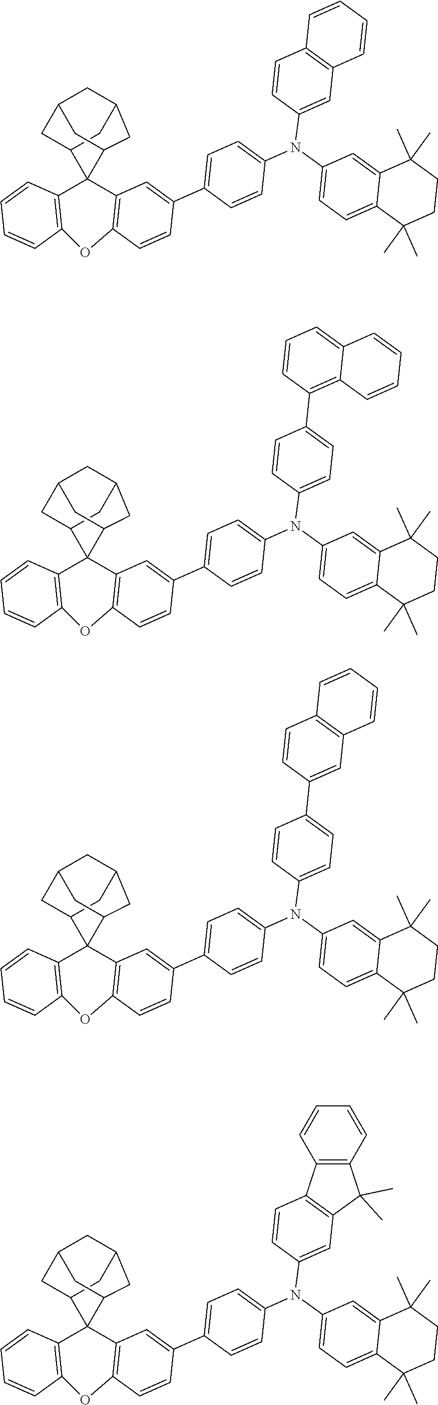 [SMILES: CC1(C)CCC(C)(C)c2cc(N(c3ccc(-c4ccc5c(c4)C4(c6ccccc6O5)C5CC6CC(C5)CC4C6)cc3)c3ccc4ccccc4c3)ccc21 ; CC1(C)CCC(C)(C)c2cc(N(c3ccc(-c4ccc5c(c4)C4(c6ccccc6O5)C5CC6CC(C5)CC4C6)cc3)c3ccc(-c4cccc5ccccc45)cc3)ccc21 ; CC1(C)CCC(C)(C)c2cc(N(c3ccc(-c4ccc5c(c4)C4(c6ccccc6O5)C5CC6CC(C5)CC4C6)cc3)c3ccc(-c4ccc5ccccc5c4)cc3)ccc21 ; CC1(C)CCC(C)(C)c2cc(N(c3ccc(-c4ccc5c(c4)C4(c6ccccc6O5)C5CC6CC(C5)CC4C6)cc3)c3ccc4c(c3)C(C)(C)c3ccccc3-4)ccc21]

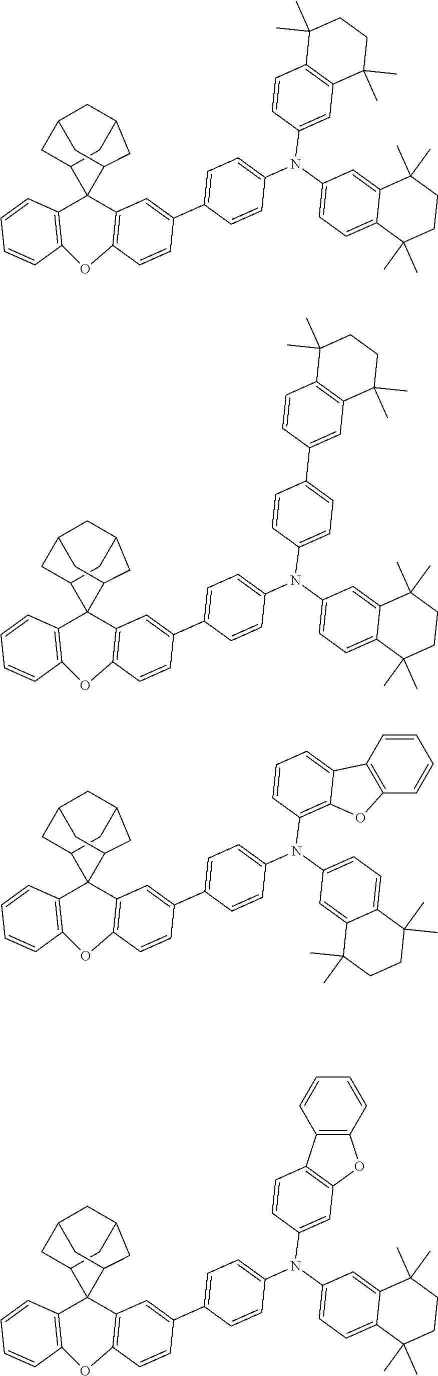

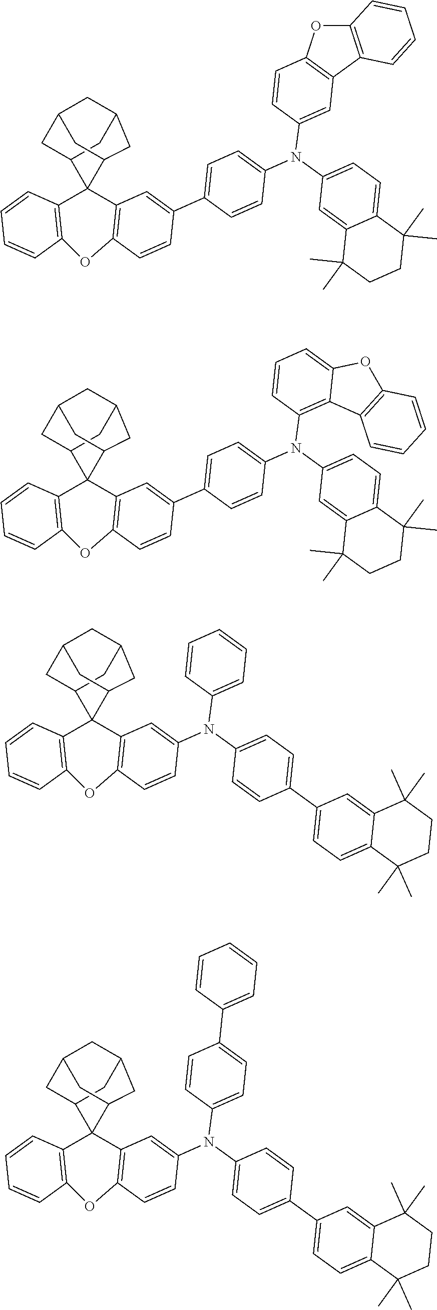 [SMILES: CC1(C)CCC(C)(C)c2cc(N(c3ccc(-c4ccc5c(c4)C4(c6ccccc6O5)C5CC6CC(C5)CC4C6)cc3)c3ccc4oc5ccccc5c4c3)ccc21 ; CC1(C)CCC(C)(C)c2cc(N(c3ccc(-c4ccc5c(c4)C4(c6ccccc6O5)C5CC6CC(C5)CC4C6)cc3)c3cccc4oc5ccccc5c34)ccc21 ; CC1(C)CCC(C)(C)c2cc(-c3ccc(N(c4ccccc4)c4ccc5c(c4)C4(c6ccccc6O5)C5CC6CC(C5)CC4C6)cc3)ccc21 ; CC1(C)CCC(C)(C)c2cc(-c3ccc(N(c4ccc(-c5ccccc5)cc4)c4ccc5c(c4)C4(c6ccccc6O5)C5CC6CC(C5)CC4C6)cc3)ccc21]

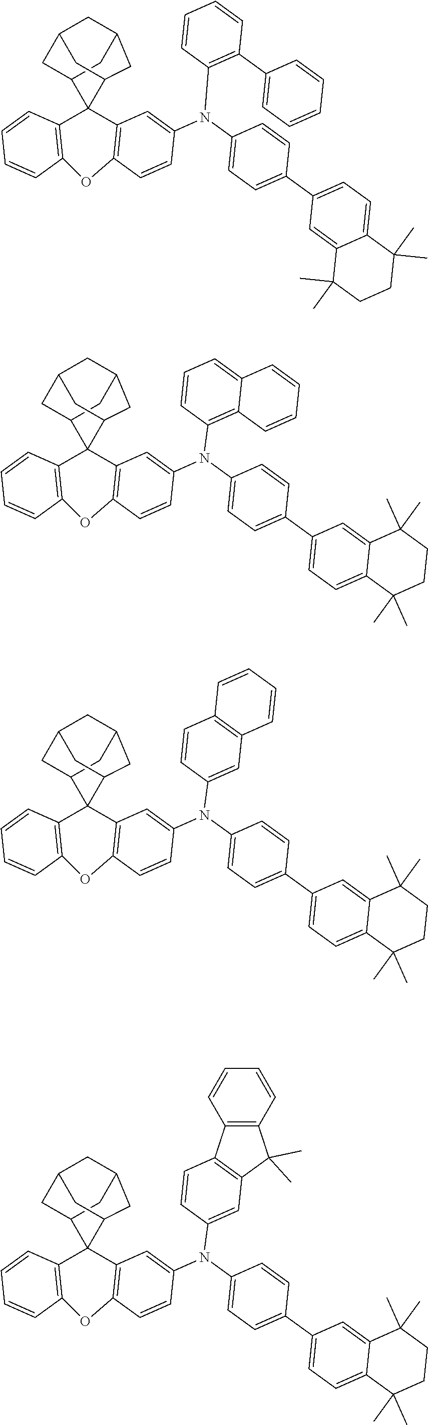 [SMILES: CC1(C)CCC(C)(C)c2cc(-c3ccc(N(c4ccc5c(c4)C4(c6ccccc6O5)C5CC6CC(C5)CC4C6)c4ccccc4-c4ccccc4)cc3)ccc21 ; CC1(C)CCC(C)(C)c2cc(-c3ccc(N(c4ccc5c(c4)C4(c6ccccc6O5)C5CC6CC(C5)CC4C6)c4cccc5ccccc45)cc3)ccc21 ; CC1(C)CCC(C)(C)c2cc(-c3ccc(N(c4ccc5c(c4)C4(c6ccccc6O5)C5CC6CC(C5)CC4C6)c4ccc5ccccc5c4)cc3)ccc21 ; CC1(C)CCC(C)(C)c2cc(-c3ccc(N(c4ccc5c(c4)C(C)(C)c4ccccc4-5)c4ccc5c(c4)C4(c6ccccc6O5)C5CC6CC(C5)CC4C6)cc3)ccc21]

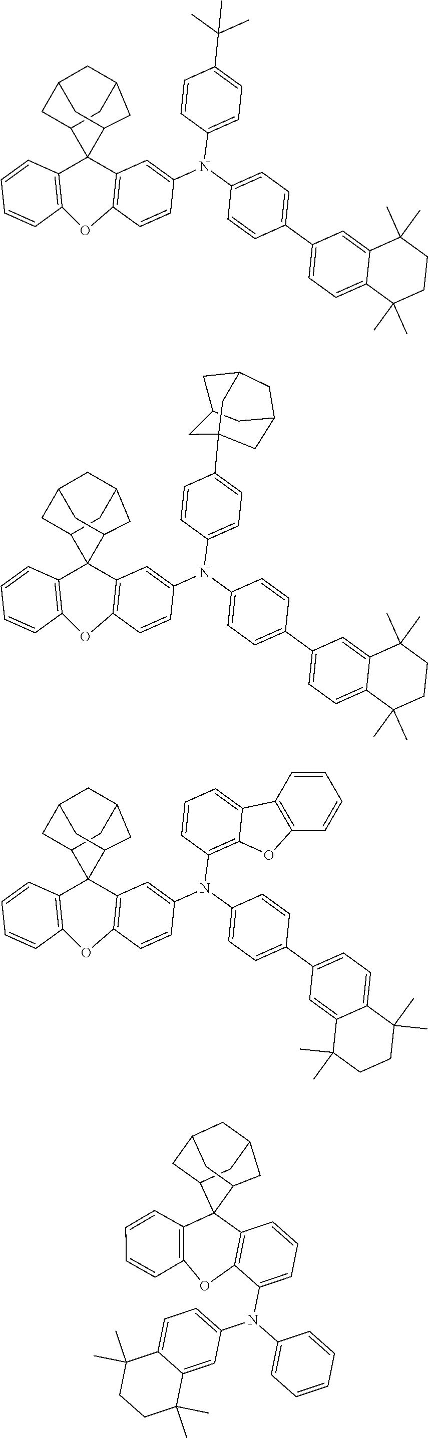

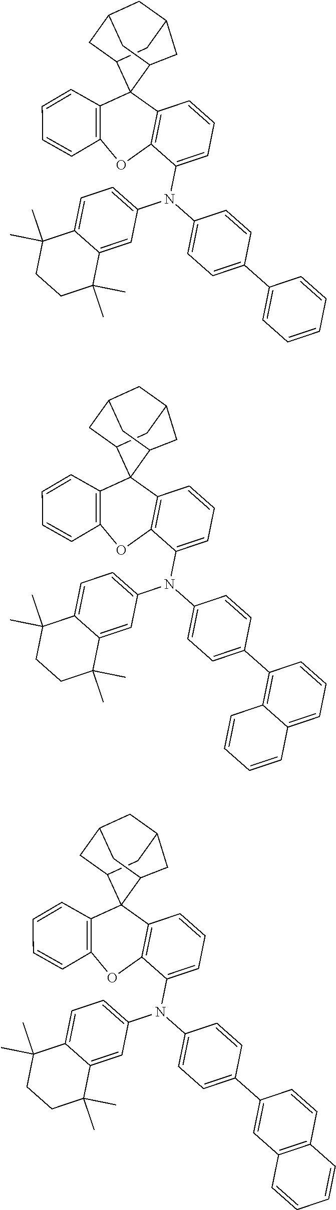 [SMILES: CC1(C)CCC(C)(C)c2cc(N(c3ccc(-c4ccccc4)cc3)c3cccc4c3Oc3ccccc3C43C4CC5CC(C4)CC3C5)ccc21 ; CC1(C)CCC(C)(C)c2cc(N(c3ccc(-c4cccc5ccccc45)cc3)c3cccc4c3Oc3ccccc3C43C4CC5CC(C4)CC3C5)ccc21 ; CC1(C)CCC(C)(C)c2cc(N(c3ccc(-c4ccc5ccccc5c4)cc3)c3cccc4c3Oc3ccccc3C43C4CC5CC(C4)CC3C5)ccc21]

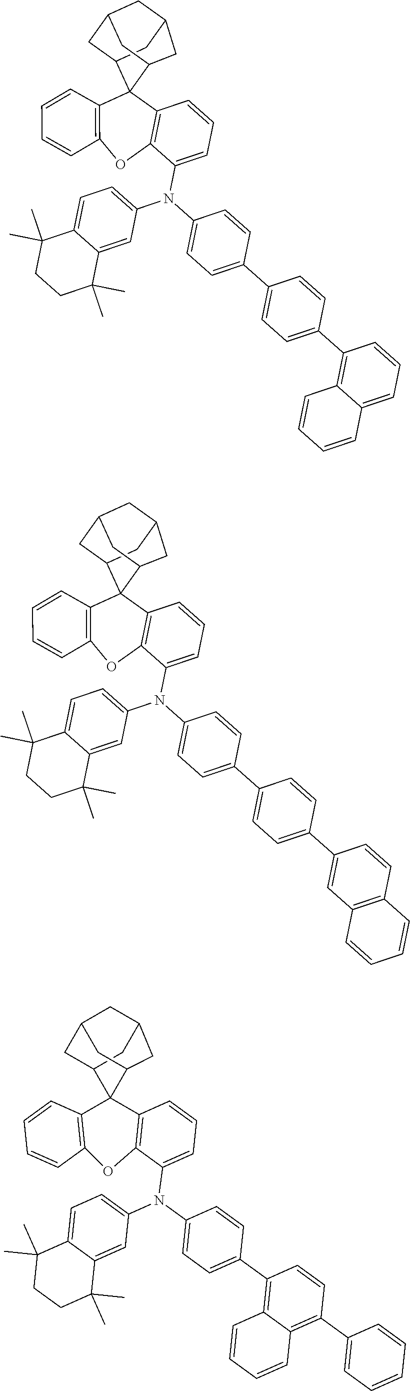 [SMILES: CC1(C)CCC(C)(C)c2cc(N(c3ccc(-c4ccc(-c5cccc6ccccc56)cc4)cc3)c3cccc4c3Oc3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3cccc4c3Oc3ccccc3C43C4CC5CC(C4)CC3C5)ccc21 ; CC1(C)CCC(C)(C)c2cc(N(c3ccc(-c4ccc(-c5ccccc5)c5ccccc45)cc3)c3cccc4c3Oc3ccccc3C43C4CC5CC(C4)CC3C5)ccc21]

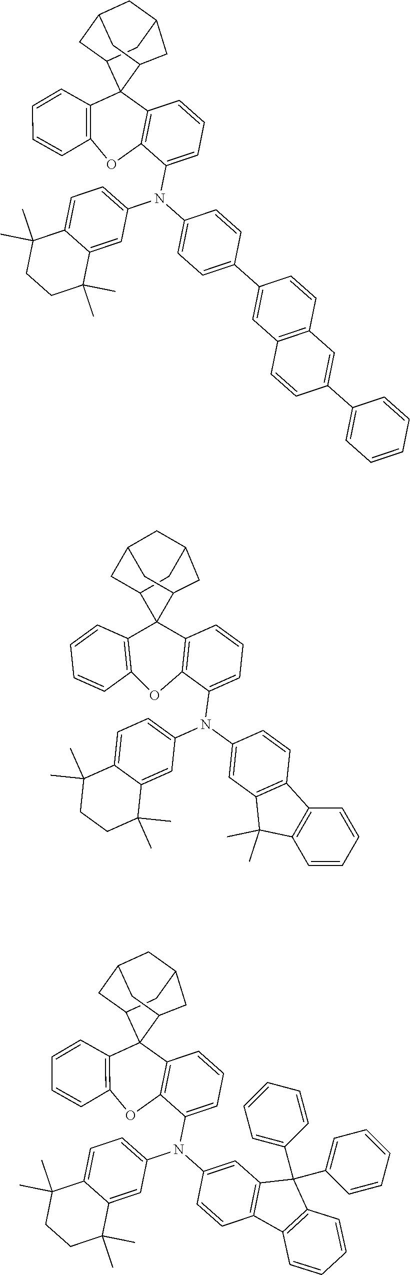 [SMILES: CC1(C)CCC(C)(C)c2cc(N(c3ccc(-c4ccc5cc(-c6ccccc6)ccc5c4)cc3)c3cccc4c3Oc3ccccc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3ccccc3C43C4CC5CC(C4)CC3C5)ccc21 ; CC1(C)CCC(C)(C)c2cc(N(c3ccc4c(c3)C(c3ccccc3)(c3ccccc3)c3ccccc3-4)c3cccc4c3Oc3ccccc3C43C4CC5CC(C4)CC3C5)ccc21]

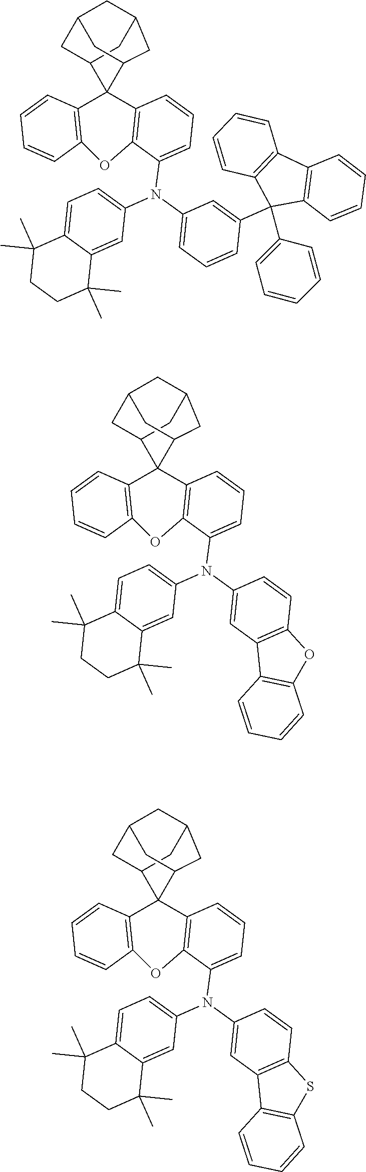 [SMILES: CC1(C)CCC(C)(C)c2cc(N(c3cccc(C4(c5ccccc5)c5ccccc5-c5ccccc54)c3)c3cccc4c3Oc3ccccc3C43C4CC5CC(C4)CC3C5)ccc21 ; CC1(C)CCC(C)(C)c2cc(N(c3ccc4oc5ccccc5c4c3)c3cccc4c3Oc3ccccc3C43C4CC5CC(C4)CC3C5)ccc21 ; CC1(C)CCC(C)(C)c2cc(N(c3ccc4sc5ccccc5c4c3)c3cccc4c3Oc3ccccc3C43C4CC5CC(C4)CC3C5)ccc21]

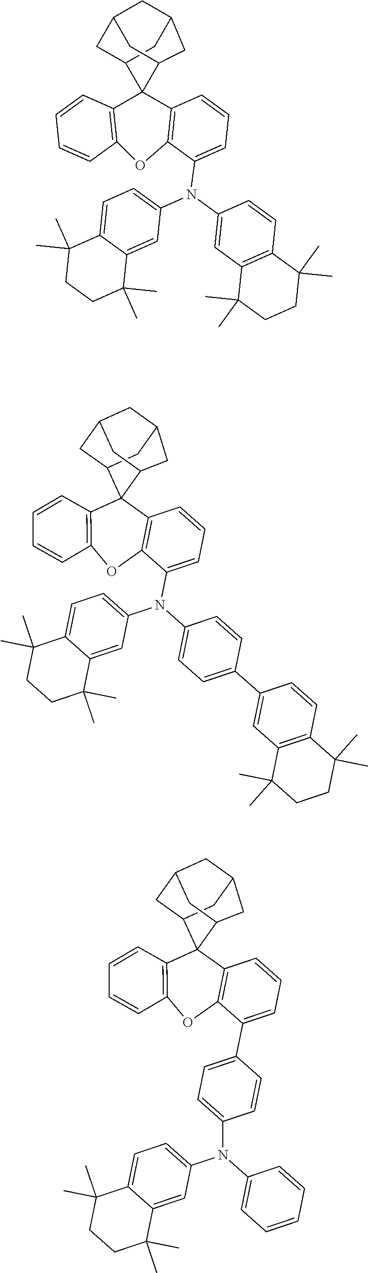

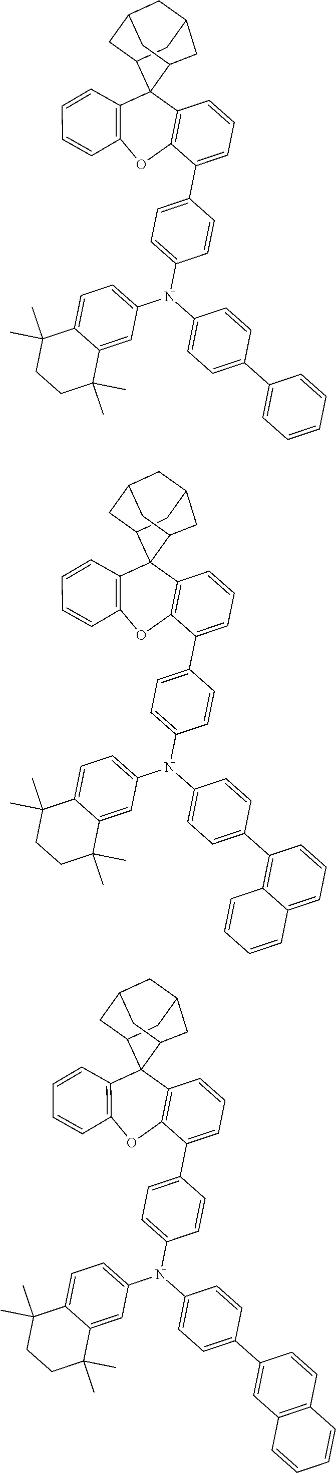 [SMILES: CC1(C)CCC(C)(C)c2cc(N(c3ccc(-c4ccccc4)cc3)c3ccc(-c4cccc5c4Oc4ccccc4C54C5CC6CC(C5)CC4C6)cc3)ccc21 ; CC1(C)CCC(C)(C)c2cc(N(c3ccc(-c4cccc5c4Oc4ccccc4C54C5CC6CC(C5)CC4C6)cc3)c3ccc(-c4cccc5ccccc45)cc3)ccc21 ; CC1(C)CCC(C)(C)c2cc(N(c3ccc(-c4ccc5ccccc5c4)cc3)c3ccc(-c4cccc5c4Oc4ccccc4C54C5CC6CC(C5)CC4C6)cc3)ccc21]

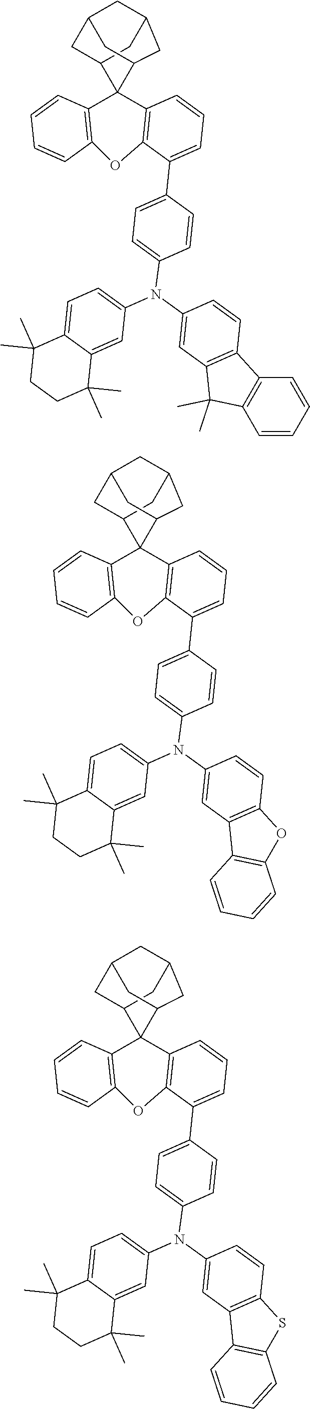 [SMILES: CC1(C)CCC(C)(C)c2cc(N(c3ccc(-c4cccc5c4Oc4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4cccc5c4Oc4ccccc4C54C5CC6CC(C5)CC4C6)cc3)c3ccc4oc5ccccc5c4c3)ccc21 ; CC1(C)CCC(C)(C)c2cc(N(c3ccc(-c4cccc5c4Oc4ccccc4C54C5CC6CC(C5)CC4C6)cc3)c3ccc4sc5ccccc5c4c3)ccc21]

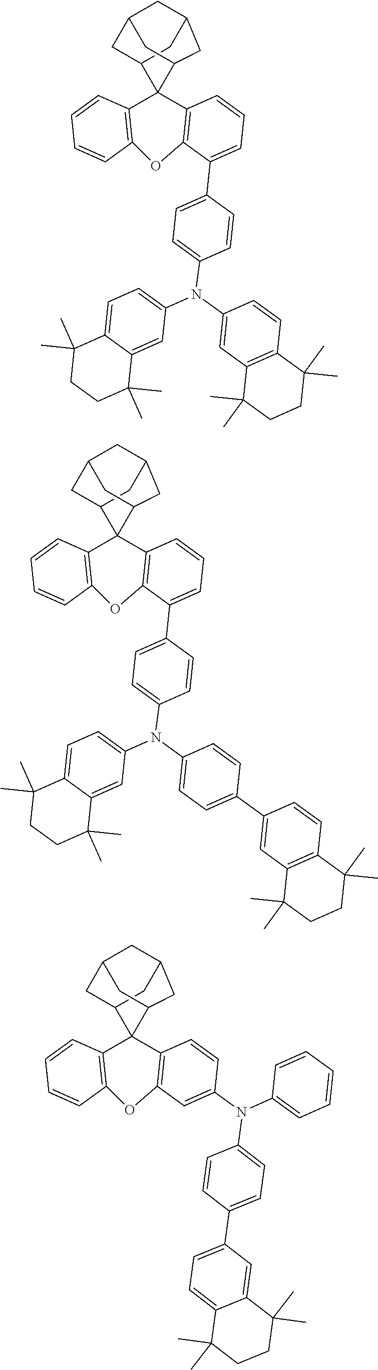

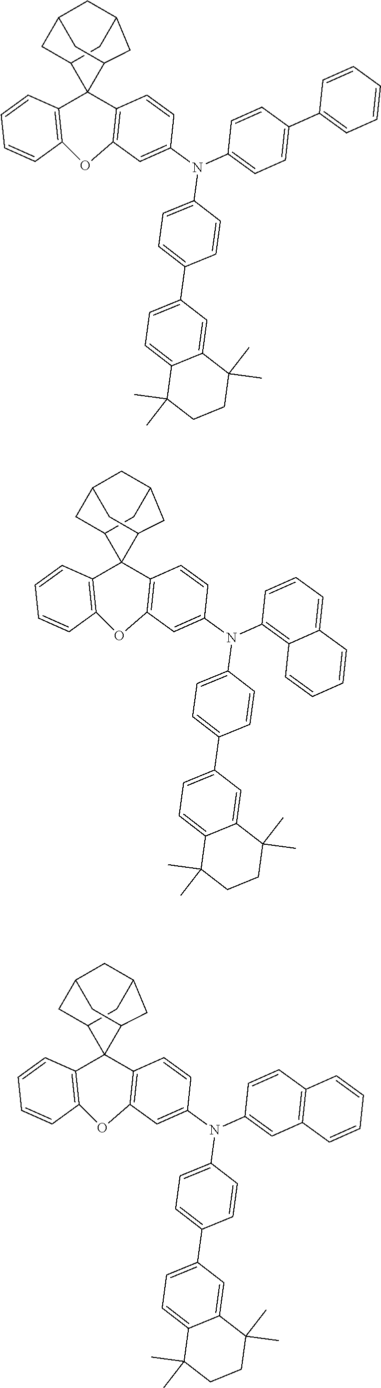 [SMILES: CC1(C)CCC(C)(C)c2cc(-c3ccc(N(c4ccc(-c5ccccc5)cc4)c4ccc5c(c4)Oc4ccccc4C54C5CC6CC(C5)CC4C6)cc3)ccc21 ; CC1(C)CCC(C)(C)c2cc(-c3ccc(N(c4ccc5c(c4)Oc4ccccc4C54C5CC6CC(C5)CC4C6)c4cccc5ccccc45)cc3)ccc21 ; CC1(C)CCC(C)(C)c2cc(-c3ccc(N(c4ccc5c(c4)Oc4ccccc4C54C5CC6CC(C5)CC4C6)c4ccc5ccccc5c4)cc3)ccc21]

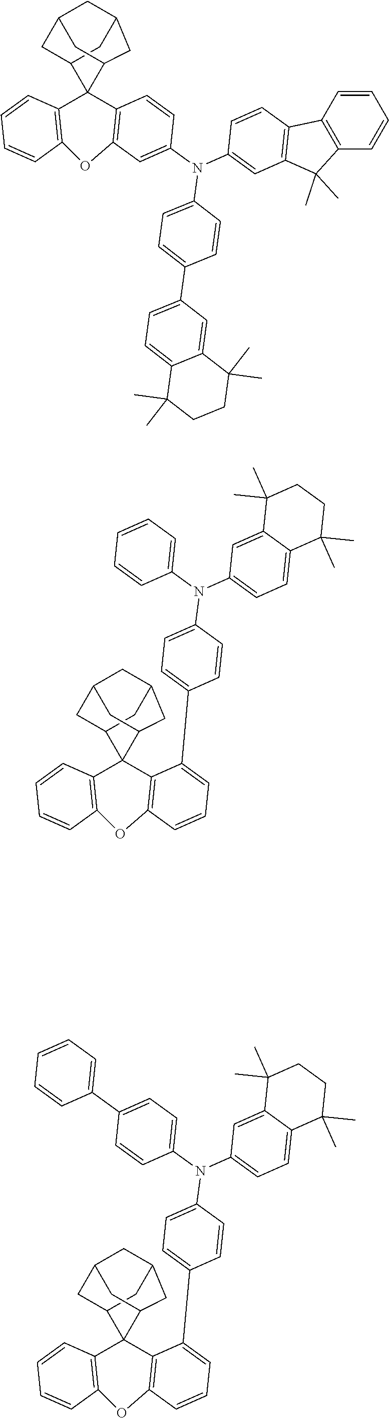 [SMILES: CC1(C)CCC(C)(C)c2cc(-c3ccc(N(c4ccc5c(c4)Oc4ccccc4C54C5CC6CC(C5)CC4C6)c4ccc5c(c4)C(C)(C)c4ccccc4-5)cc3)ccc21 ; CC1(C)CCC(C)(C)c2cc(N(c3ccccc3)c3ccc(-c4cccc5c4C4(c6ccccc6O5)C5CC6CC(C5)CC4C6)cc3)ccc21 ; CC1(C)CCC(C)(C)c2cc(N(c3ccc(-c4ccccc4)cc3)c3ccc(-c4cccc5c4C4(c6ccccc6O5)C5CC6CC(C5)CC4C6)cc3)ccc21]

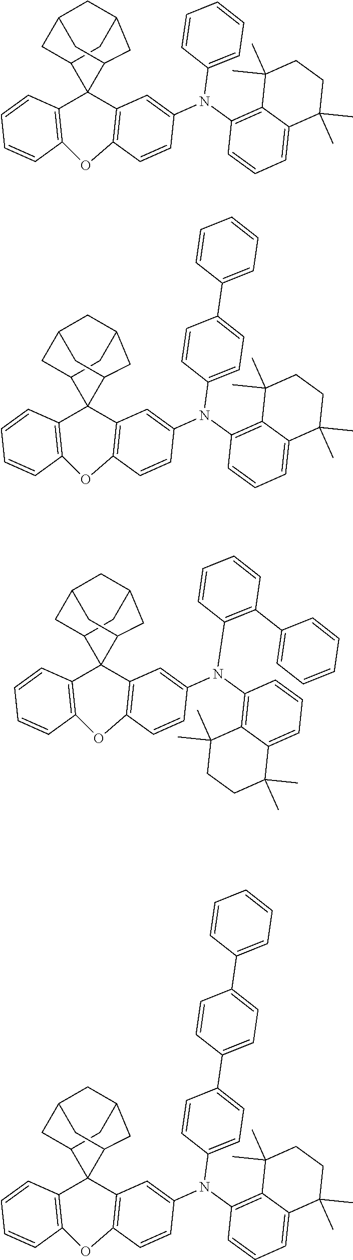 [SMILES: CC1(C)CCC(C)(C)c2c(N(c3ccccc3)c3ccc4c(c3)C3(c5ccccc5O4)C4CC5CC(C4)CC3C5)cccc21 ; CC1(C)CCC(C)(C)c2c(N(c3ccc(-c4ccccc4)cc3)c3ccc4c(c3)C3(c5ccccc5O4)C4CC5CC(C4)CC3C5)cccc21 ; CC1(C)CCC(C)(C)c2c(N(c3ccc4c(c3)C3(c5ccccc5O4)C4CC5CC(C4)CC3C5)c3ccccc3-c3ccccc3)cccc21 ; CC1(C)CCC(C)(C)c2c(N(c3ccc(-c4ccc(-c5ccccc5)cc4)cc3)c3ccc4c(c3)C3(c5ccccc5O4)C4CC5CC(C4)CC3C5)cccc21]

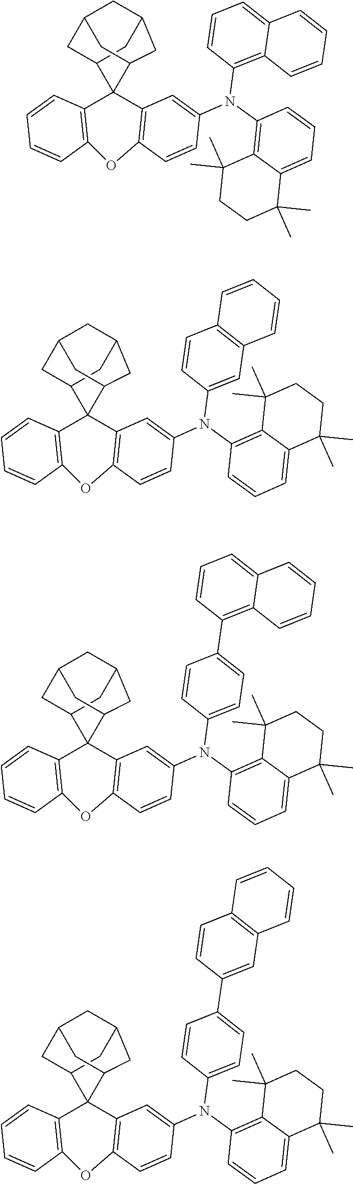

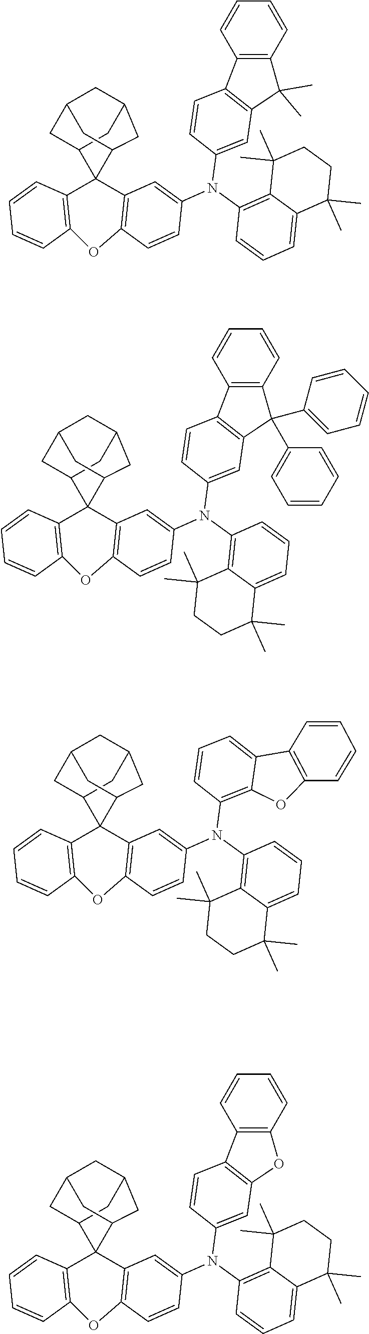 [SMILES: CC1(C)CCC(C)(C)c2c(N(c3ccc4c(c3)C(C)(C)c3ccccc3-4)c3ccc4c(c3)C3(c5ccccc5O4)C4CC5CC(C4)CC3C5)cccc21 ; CC1(C)CCC(C)(C)c2c(N(c3ccc4c(c3)C(c3ccccc3)(c3ccccc3)c3ccccc3-4)c3ccc4c(c3)C3(c5ccccc5O4)C4CC5CC(C4)CC3C5)cccc21 ; CC1(C)CCC(C)(C)c2c(N(c3ccc4c(c3)C3(c5ccccc5O4)C4CC5CC(C4)CC3C5)c3cccc4c3oc3ccccc34)cccc21 ; CC1(C)CCC(C)(C)c2c(N(c3ccc4c(c3)C3(c5ccccc5O4)C4CC5CC(C4)CC3C5)c3ccc4c(c3)oc3ccccc34)cccc21]

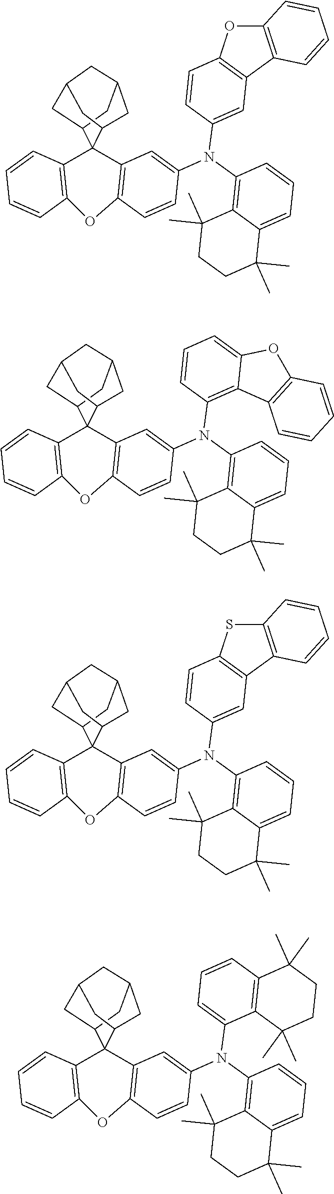

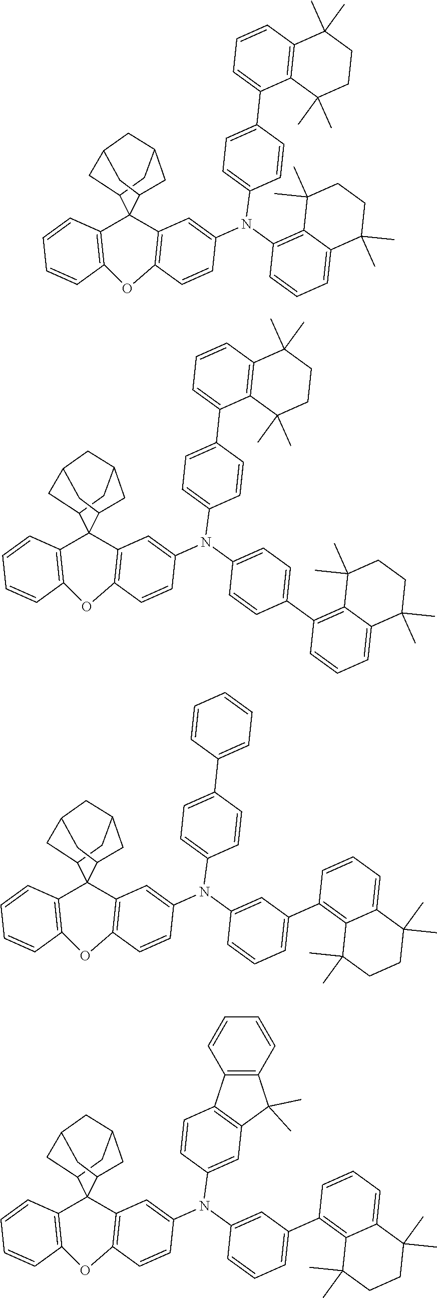

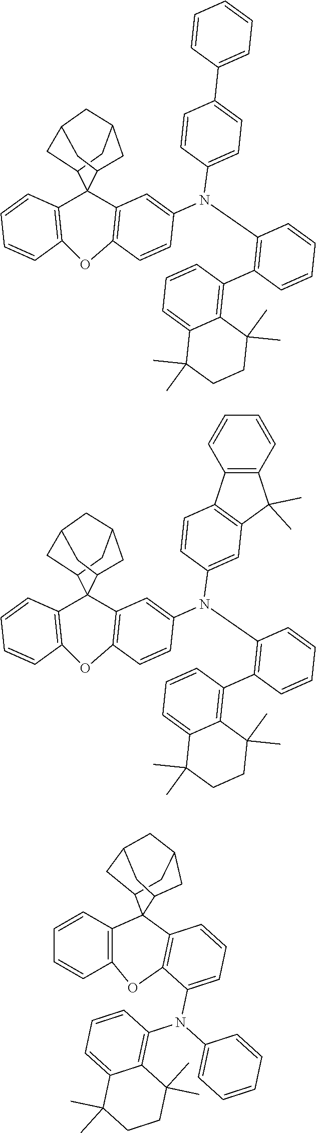

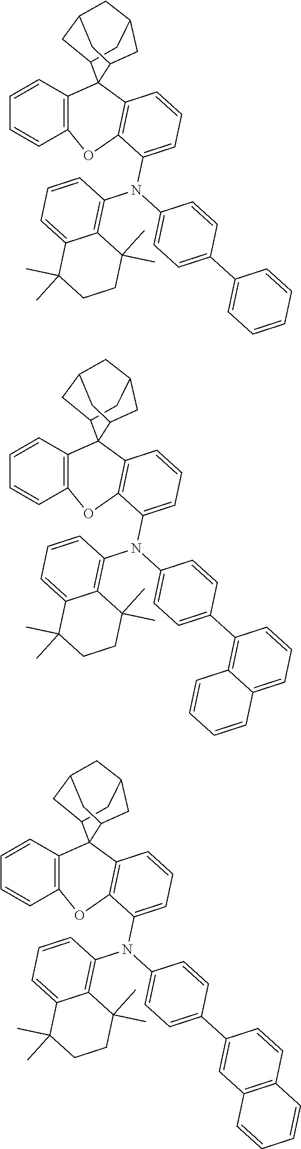 [SMILES: CC1(C)CCC(C)(C)c2c(N(c3ccc(-c4ccccc4)cc3)c3cccc4c3Oc3ccccc3C43C4CC5CC(C4)CC3C5)cccc21 ; CC1(C)CCC(C)(C)c2c(N(c3ccc(-c4cccc5ccccc45)cc3)c3cccc4c3Oc3ccccc3C43C4CC5CC(C4)CC3C5)cccc21 ; CC1(C)CCC(C)(C)c2c(N(c3ccc(-c4ccc5ccccc5c4)cc3)c3cccc4c3Oc3ccccc3C43C4CC5CC(C4)CC3C5)cccc21]

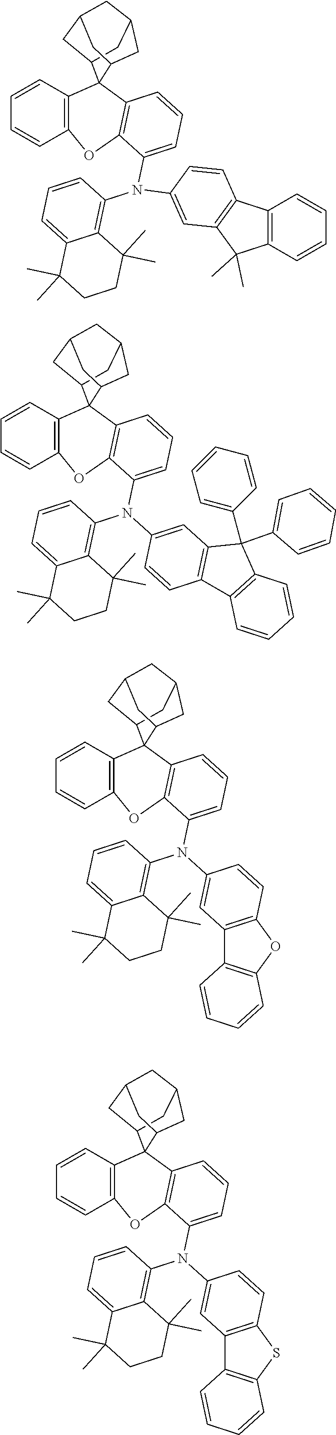

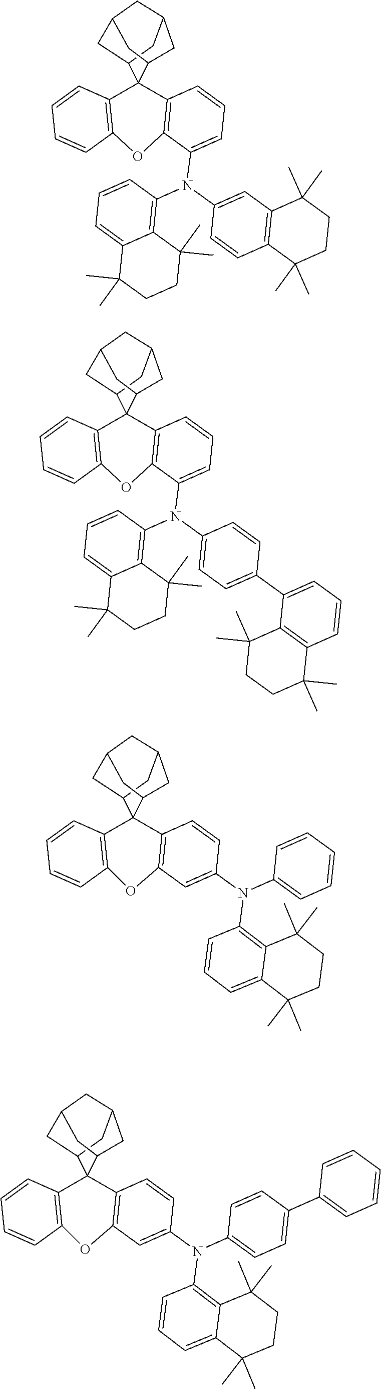

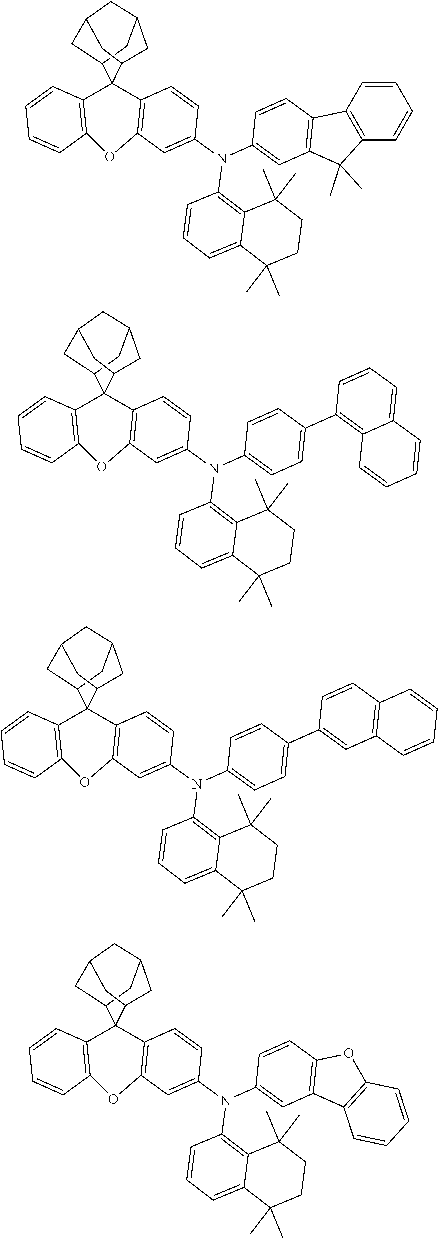 [SMILES: CC1(C)CCC(C)(C)c2c(N(c3ccc4c(c3)Oc3ccccc3C43C4CC5CC(C4)CC3C5)c3ccc4c(c3)C(C)(C)c3ccccc3-4)cccc21 ; CC1(C)CCC(C)(C)c2c(N(c3ccc(-c4cccc5ccccc45)cc3)c3ccc4c(c3)Oc3ccccc3C43C4CC5CC(C4)CC3C5)cccc21 ; CC1(C)CCC(C)(C)c2c(N(c3ccc(-c4ccc5ccccc5c4)cc3)c3ccc4c(c3)Oc3ccccc3C43C4CC5CC(C4)CC3C5)cccc21 ; CC1(C)CCC(C)(C)c2c(N(c3ccc4c(c3)Oc3ccccc3C43C4CC5CC(C4)CC3C5)c3ccc4oc5ccccc5c4c3)cccc21]

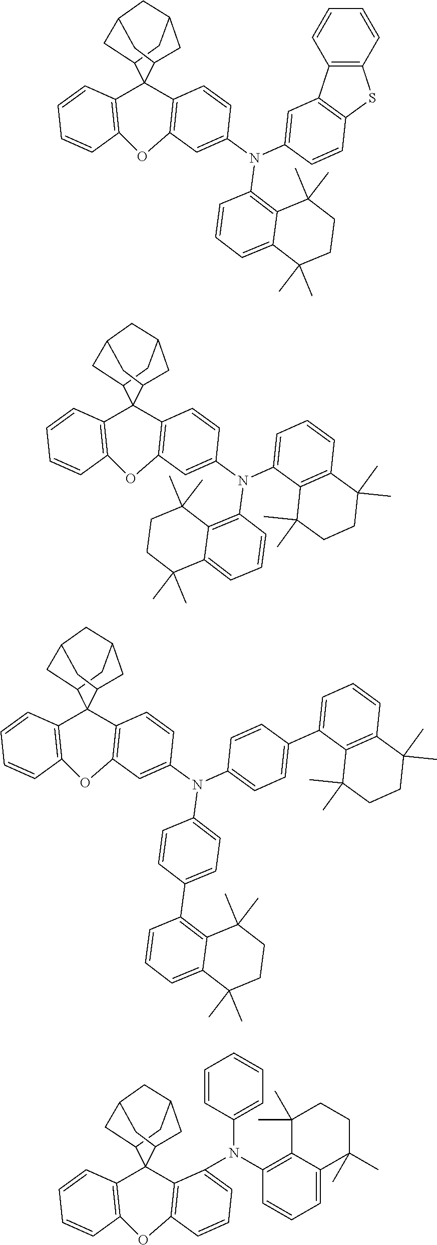

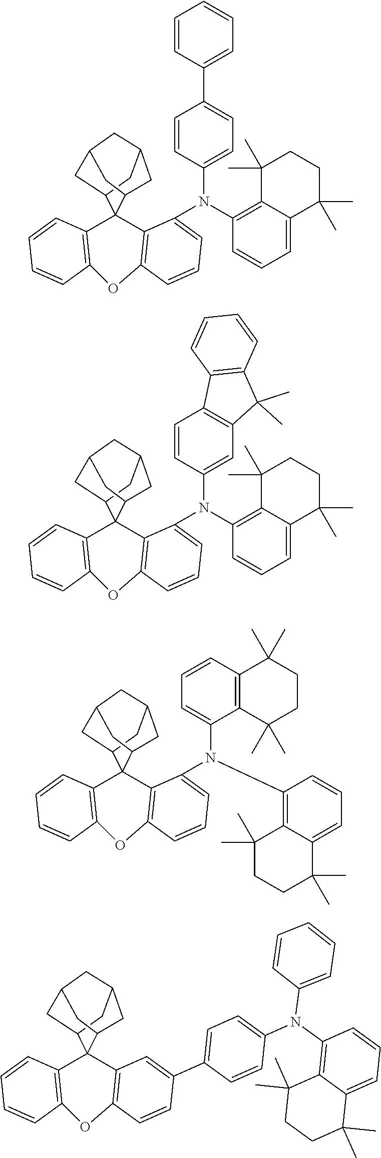

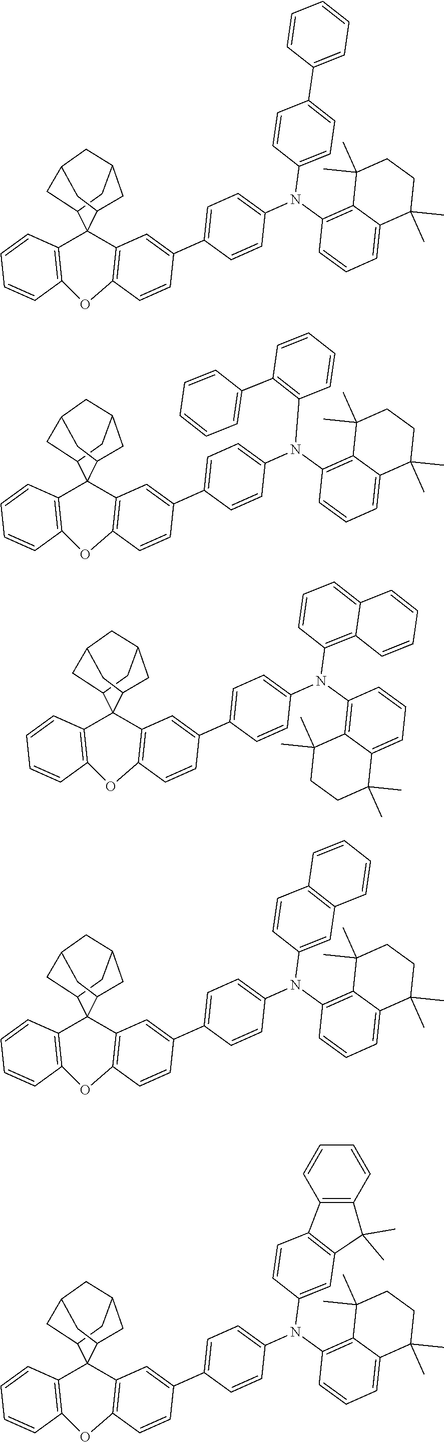

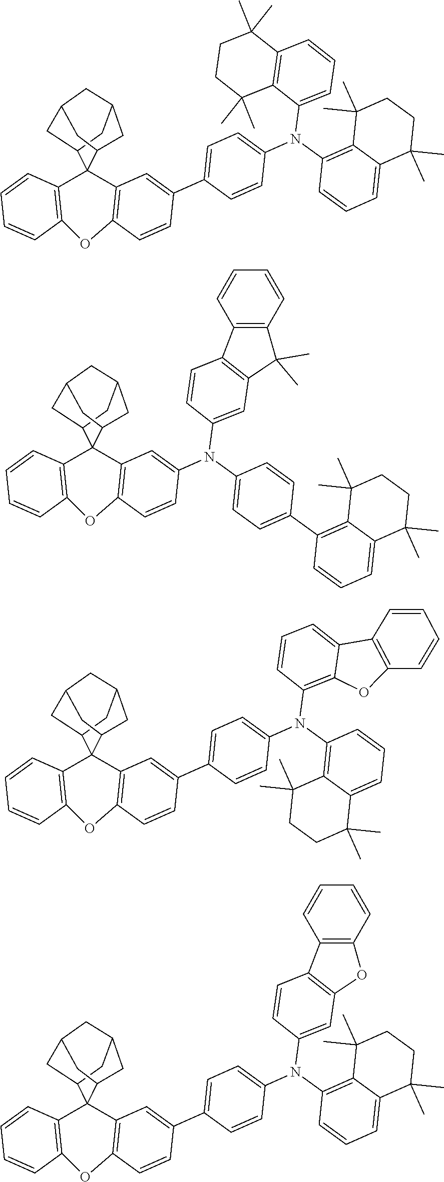

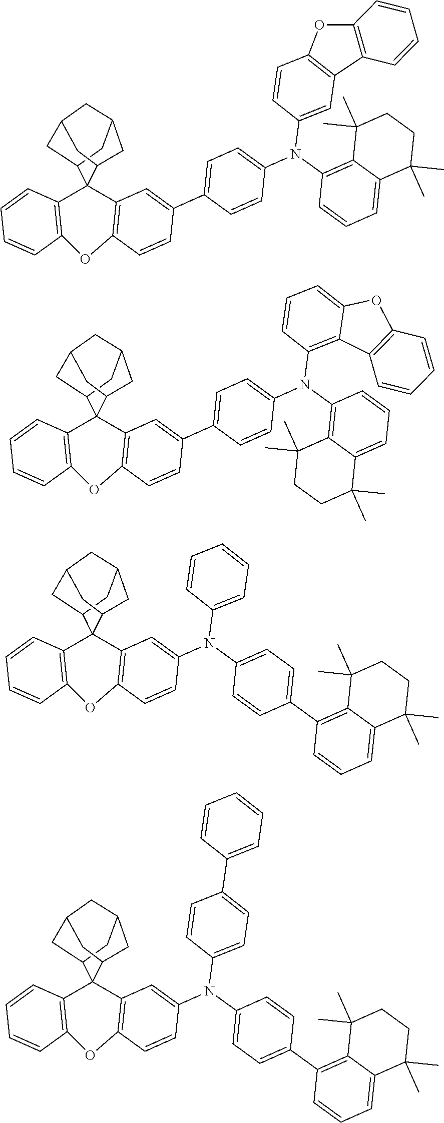 [SMILES: CC1(C)CCC(C)(C)c2c(N(c3ccc(-c4ccc5c(c4)C4(c6ccccc6O5)C5CC6CC(C5)CC4C6)cc3)c3ccc4oc5ccccc5c4c3)cccc21 ; CC1(C)CCC(C)(C)c2c(N(c3ccc(-c4ccc5c(c4)C4(c6ccccc6O5)C5CC6CC(C5)CC4C6)cc3)c3cccc4oc5ccccc5c34)cccc21 ; CC1(C)CCC(C)(C)c2c(-c3ccc(N(c4ccccc4)c4ccc5c(c4)C4(c6ccccc6O5)C5CC6CC(C5)CC4C6)cc3)cccc21 ; CC1(C)CCC(C)(C)c2c(-c3ccc(N(c4ccc(-c5ccccc5)cc4)c4ccc5c(c4)C4(c6ccccc6O5)C5CC6CC(C5)CC4C6)cc3)cccc21]

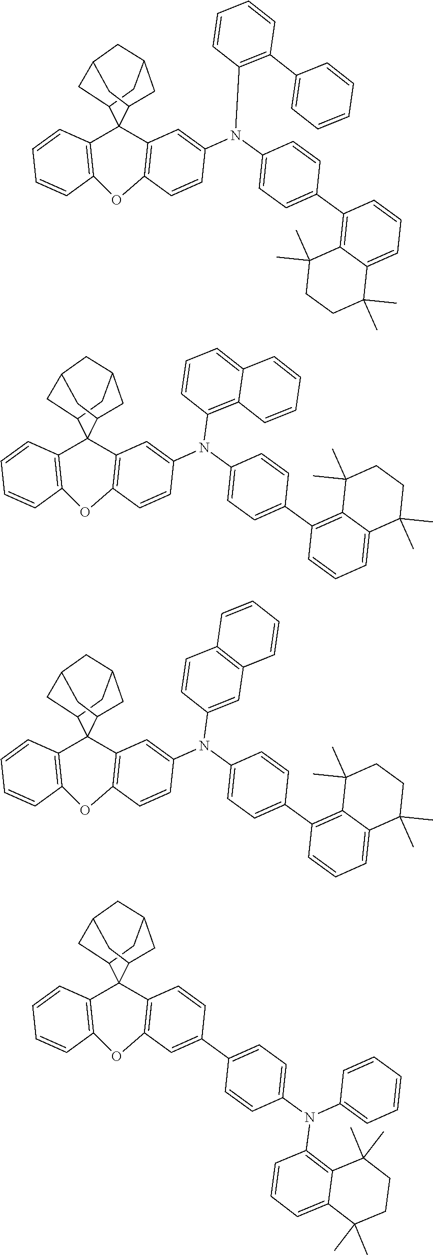 [SMILES: CC1(C)CCC(C)(C)c2c(-c3ccc(N(c4ccc5c(c4)C4(c6ccccc6O5)C5CC6CC(C5)CC4C6)c4ccccc4-c4ccccc4)cc3)cccc21 ; CC1(C)CCC(C)(C)c2c(-c3ccc(N(c4ccc5c(c4)C4(c6ccccc6O5)C5CC6CC(C5)CC4C6)c4cccc5ccccc45)cc3)cccc21 ; CC1(C)CCC(C)(C)c2c(-c3ccc(N(c4ccc5c(c4)C4(c6ccccc6O5)C5CC6CC(C5)CC4C6)c4ccc5ccccc5c4)cc3)cccc21 ; CC1(C)CCC(C)(C)c2c(N(c3ccccc3)c3ccc(-c4ccc5c(c4)Oc4ccccc4C54C5CC6CC(C5)CC4C6)cc3)cccc21]

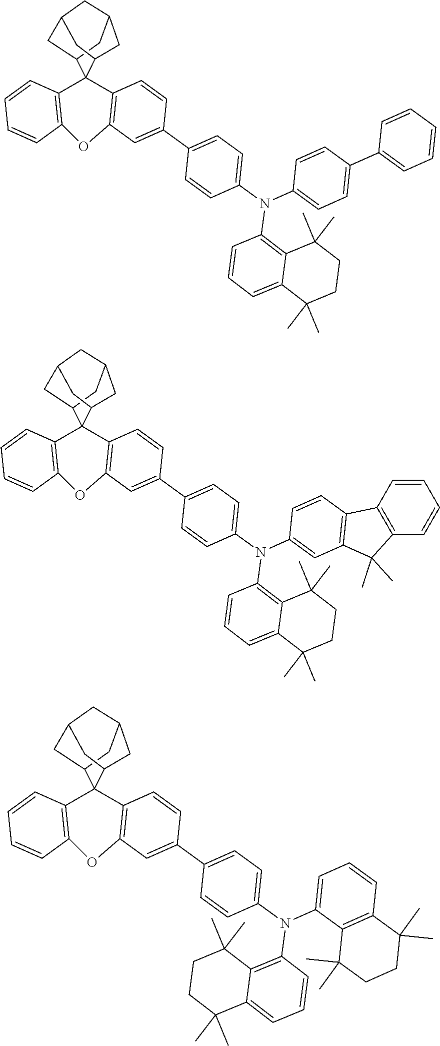 [SMILES: CC1(C)CCC(C)(C)c2c(N(c3ccc(-c4ccccc4)cc3)c3ccc(-c4ccc5c(c4)Oc4ccccc4C54C5CC6CC(C5)CC4C6)cc3)cccc21 ; CC1(C)CCC(C)(C)c2c(N(c3ccc(-c4ccc5c(c4)Oc4ccccc4C54C5CC6CC(C5)CC4C6)cc3)c3ccc4c(c3)C(C)(C)c3ccccc3-4)cccc21 ; CC1(C)CCC(C)(C)c2c(N(c3ccc(-c4ccc5c(c4)Oc4ccccc4C54C5CC6CC(C5)CC4C6)cc3)c3cccc4c3C(C)(C)CCC4(C)C)cccc21]

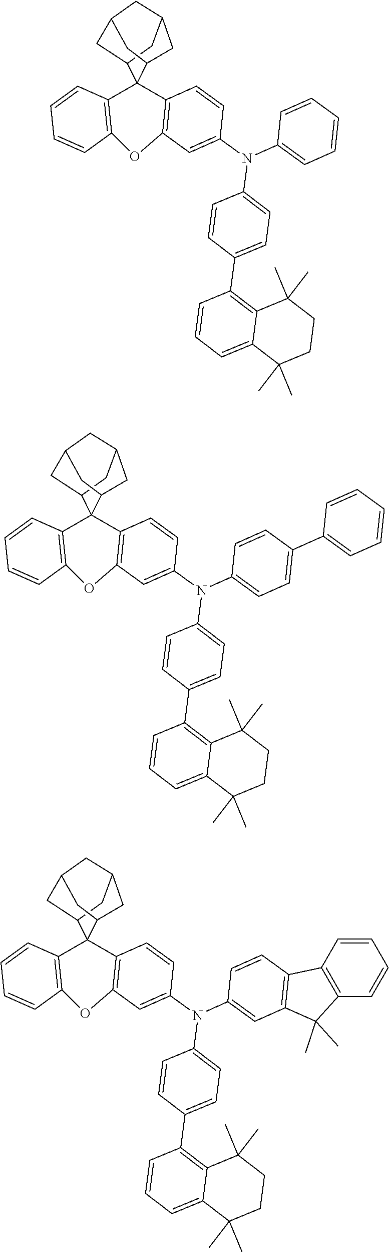 [SMILES: CC1(C)CCC(C)(C)c2c(-c3ccc(N(c4ccccc4)c4ccc5c(c4)Oc4ccccc4C54C5CC6CC(C5)CC4C6)cc3)cccc21 ; CC1(C)CCC(C)(C)c2c(-c3ccc(N(c4ccc(-c5ccccc5)cc4)c4ccc5c(c4)Oc4ccccc4C54C5CC6CC(C5)CC4C6)cc3)cccc21 ; CC1(C)CCC(C)(C)c2c(-c3ccc(N(c4ccc5c(c4)Oc4ccccc4C54C5CC6CC(C5)CC4C6)c4ccc5c(c4)C(C)(C)c4ccccc4-5)cc3)cccc21]

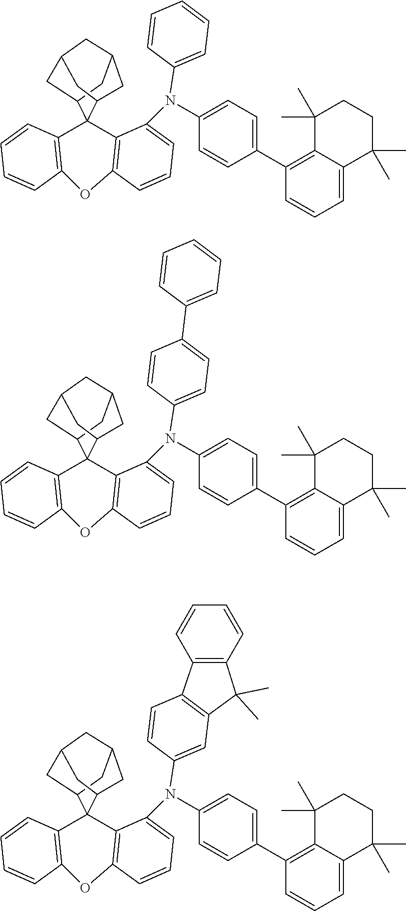 [SMILES: CC1(C)CCC(C)(C)c2c(-c3ccc(N(c4ccccc4)c4cccc5c4C4(c6ccccc6O5)C5CC6CC(C5)CC4C6)cc3)cccc21 ; CC1(C)CCC(C)(C)c2c(-c3ccc(N(c4ccc(-c5ccccc5)cc4)c4cccc5c4C4(c6ccccc6O5)C5CC6CC(C5)CC4C6)cc3)cccc21 ; CC1(C)CCC(C)(C)c2c(-c3ccc(N(c4ccc5c(c4)C(C)(C)c4ccccc4-5)c4cccc5c4C4(c6ccccc6O5)C5CC6CC(C5)CC4C6)cc3)cccc21]

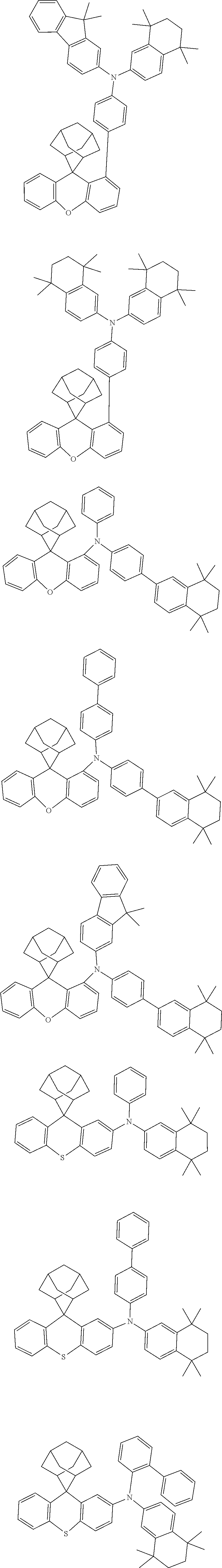

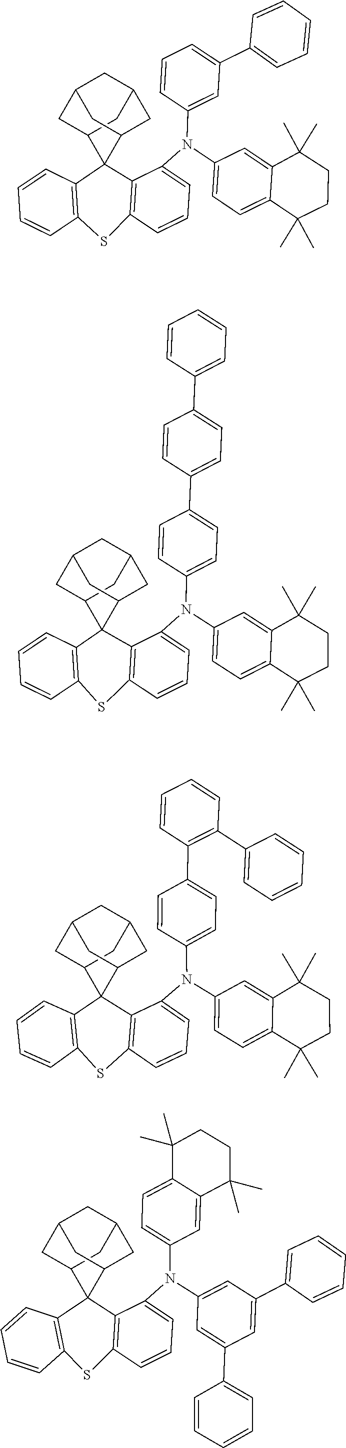

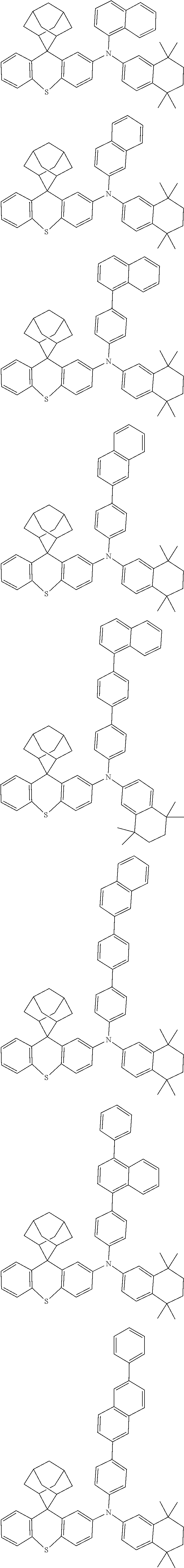

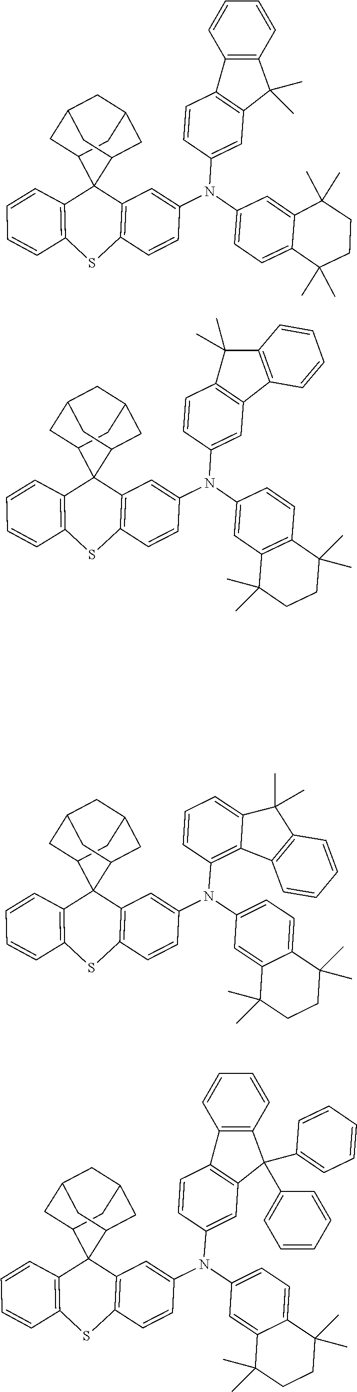

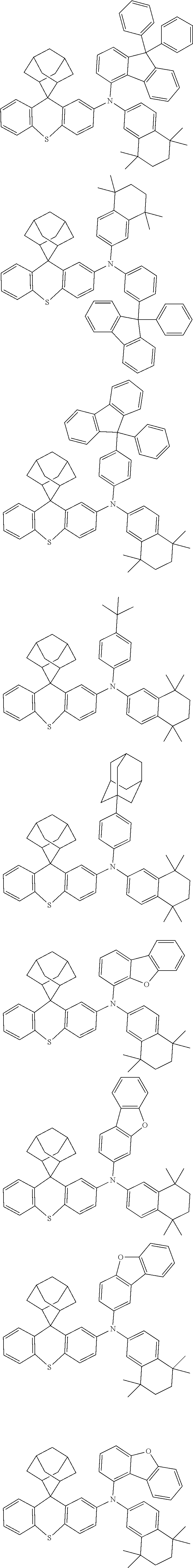

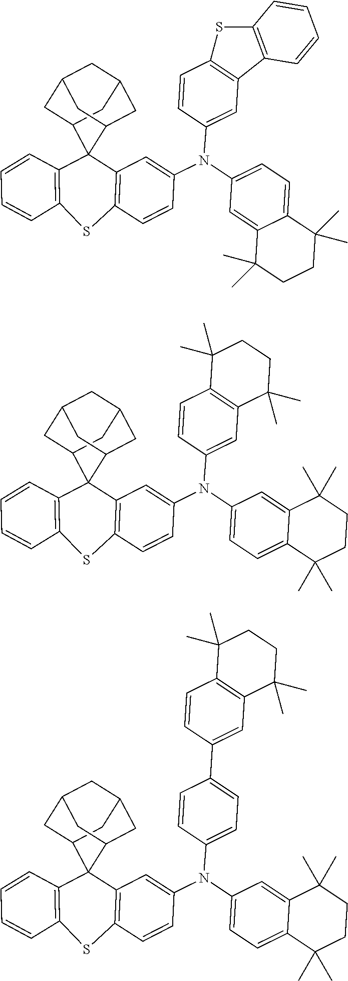

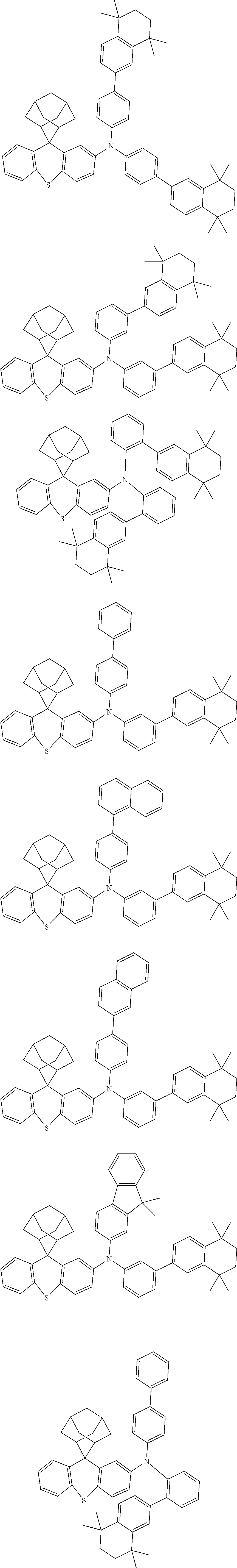

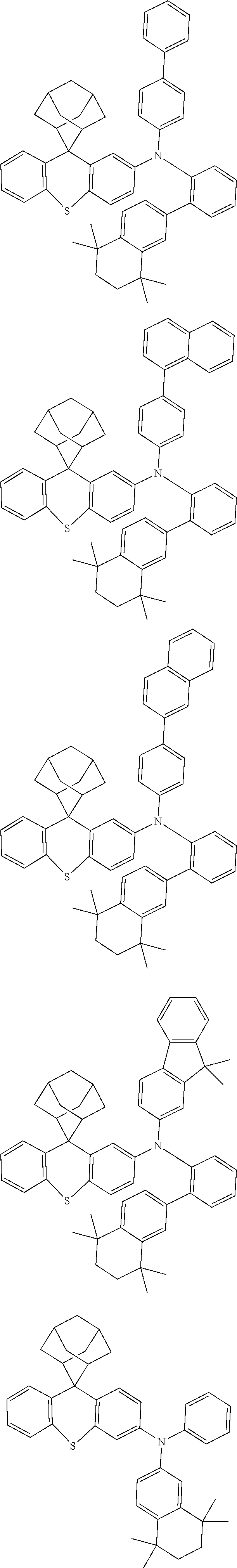

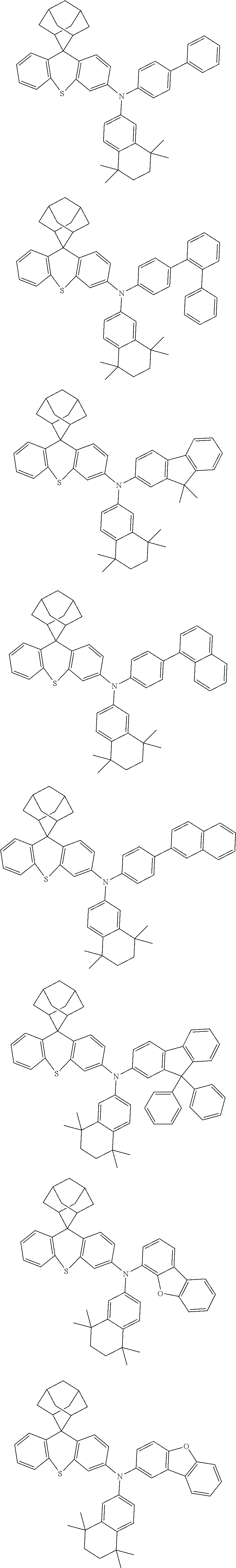

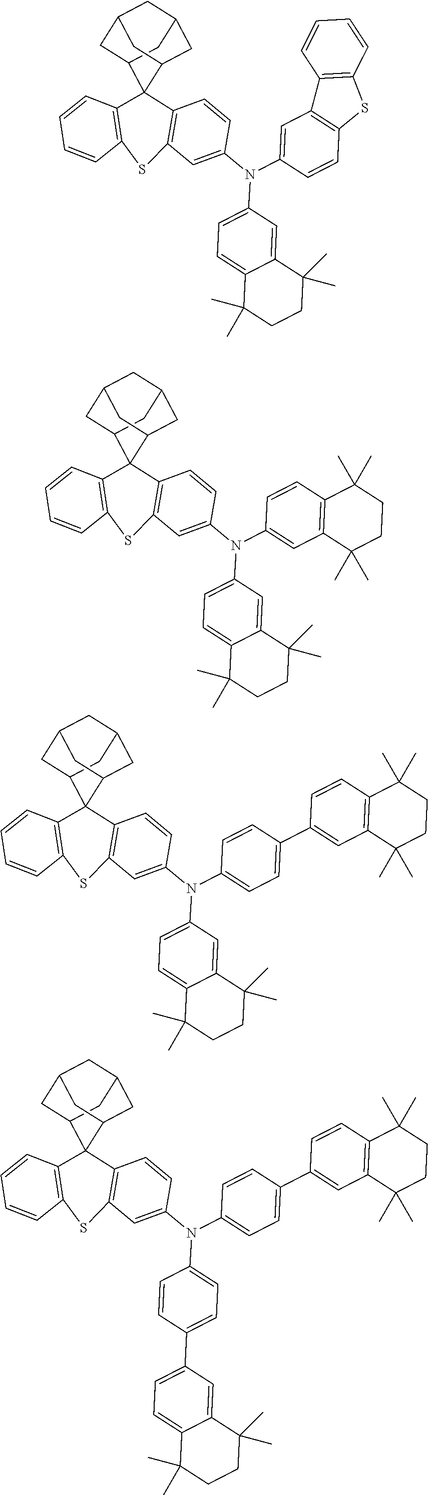 [SMILES: CC1(C)CCC(C)(C)c2cc(N(c3ccc4c(c3)Sc3ccccc3C43C4CC5CC(C4)CC3C5)c3ccc4sc5ccccc5c4c3)ccc21 ; CC1(C)CCC(C)(C)c2cc(N(c3ccc4c(c3)Sc3ccccc3C43C4CC5CC(C4)CC3C5)c3ccc4c(c3)C(C)(C)CCC4(C)C)ccc21 ; CC1(C)CCC(C)(C)c2cc(-c3ccc(N(c4ccc5c(c4)Sc4ccccc4C54C5CC6CC(C5)CC4C6)c4ccc5c(c4)C(C)(C)CCC5(C)C)cc3)ccc21 ; CC1(C)CCC(C)(C)c2cc(-c3ccc(N(c4ccc(-c5ccc6c(c5)C(C)(C)CCC6(C)C)cc4)c4ccc5c(c4)Sc4ccccc4C54C5CC6CC(C5)CC4C6)cc3)ccc21]

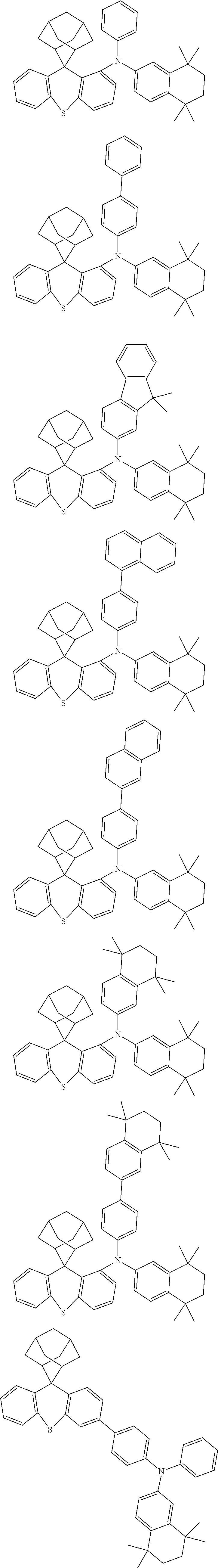

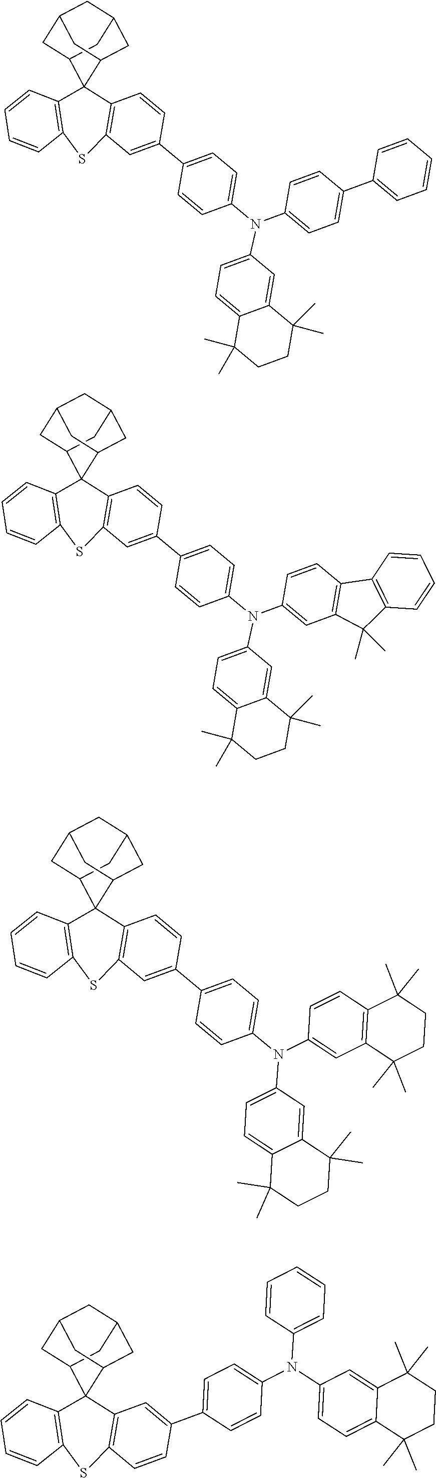 [SMILES: CC1(C)CCC(C)(C)c2cc(N(c3ccc(-c4ccccc4)cc3)c3ccc(-c4ccc5c(c4)Sc4ccccc4C54C5CC6CC(C5)CC4C6)cc3)ccc21 ; CC1(C)CCC(C)(C)c2cc(N(c3ccc(-c4ccc5c(c4)Sc4ccccc4C54C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc5c(c4)Sc4ccccc4C54C5CC6CC(C5)CC4C6)cc3)c3ccc4c(c3)C(C)(C)CCC4(C)C)ccc21 ; CC1(C)CCC(C)(C)c2cc(N(c3ccccc3)c3ccc(-c4ccc5c(c4)C4(c6ccccc6S5)C5CC6CC(C5)CC4C6)cc3)ccc21]

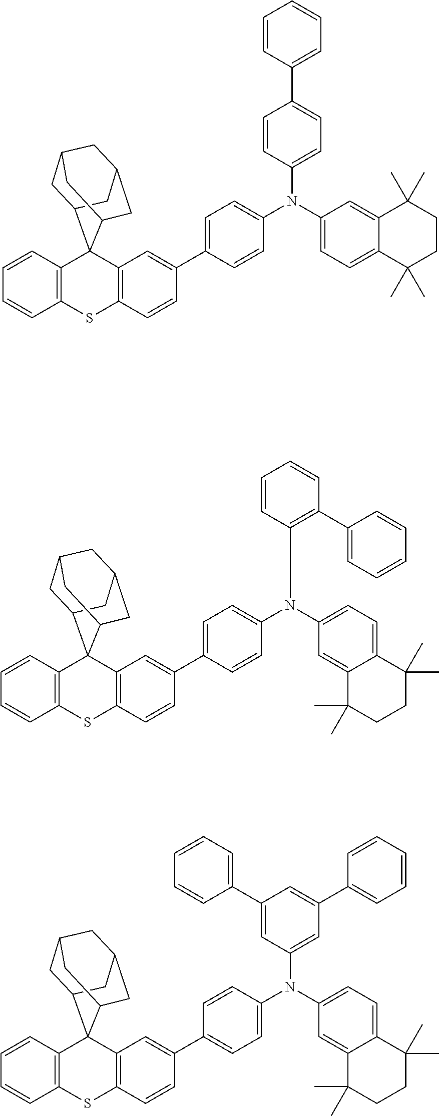 [SMILES: CC1(C)CCC(C)(C)c2cc(N(c3ccc(-c4ccccc4)cc3)c3ccc(-c4ccc5c(c4)C4(c6ccccc6S5)C5CC6CC(C5)CC4C6)cc3)ccc21 ; CC1(C)CCC(C)(C)c2cc(N(c3ccc(-c4ccc5c(c4)C4(c6ccccc6S5)C5CC6CC(C5)CC4C6)cc3)c3ccccc3-c3ccccc3)ccc21 ; CC1(C)CCC(C)(C)c2cc(N(c3ccc(-c4ccc5c(c4)C4(c6ccccc6S5)C5CC6CC(C5)CC4C6)cc3)c3cc(-c4ccccc4)cc(-c4ccccc4)c3)ccc21]

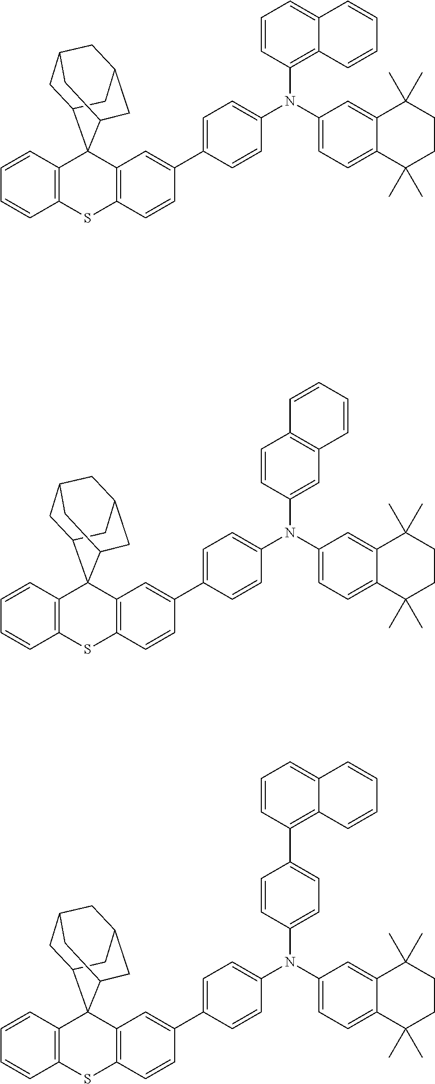 [SMILES: CC1(C)CCC(C)(C)c2cc(N(c3ccc(-c4ccc5c(c4)C4(c6ccccc6S5)C5CC6CC(C5)CC4C6)cc3)c3cccc4ccccc34)ccc21 ; CC1(C)CCC(C)(C)c2cc(N(c3ccc(-c4ccc5c(c4)C4(c6ccccc6S5)C5CC6CC(C5)CC4C6)cc3)c3ccc4ccccc4c3)ccc21 ; CC1(C)CCC(C)(C)c2cc(N(c3ccc(-c4ccc5c(c4)C4(c6ccccc6S5)C5CC6CC(C5)CC4C6)cc3)c3ccc(-c4cccc5ccccc45)cc3)ccc21]

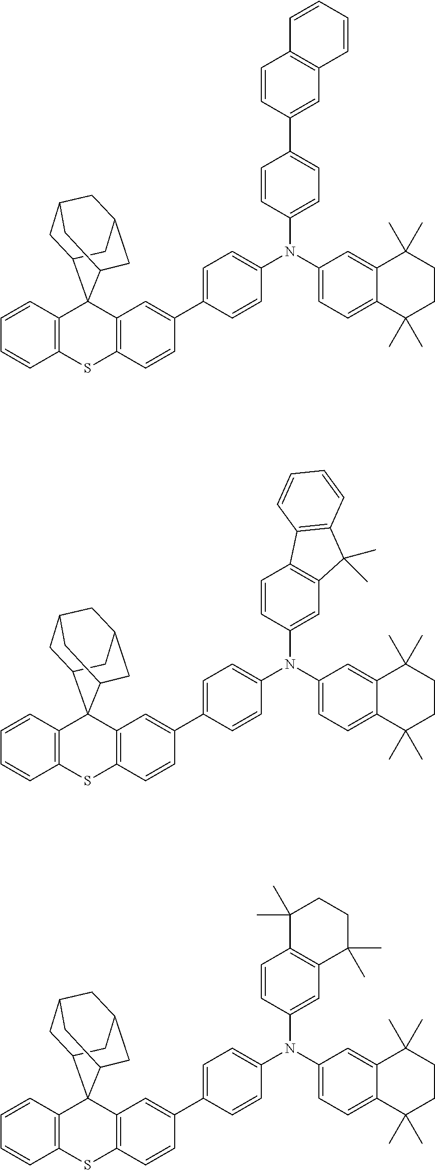 [SMILES: CC1(C)CCC(C)(C)c2cc(N(c3ccc(-c4ccc5c(c4)C4(c6ccccc6S5)C5CC6CC(C5)CC4C6)cc3)c3ccc(-c4ccc5ccccc5c4)cc3)ccc21 ; CC1(C)CCC(C)(C)c2cc(N(c3ccc(-c4ccc5c(c4)C4(c6ccccc6S5)C5CC6CC(C5)CC4C6)cc3)c3ccc4c(c3)C(C)(C)c3ccccc3-4)ccc21 ; CC1(C)CCC(C)(C)c2cc(N(c3ccc(-c4ccc5c(c4)C4(c6ccccc6S5)C5CC6CC(C5)CC4C6)cc3)c3ccc4c(c3)C(C)(C)CCC4(C)C)ccc21]

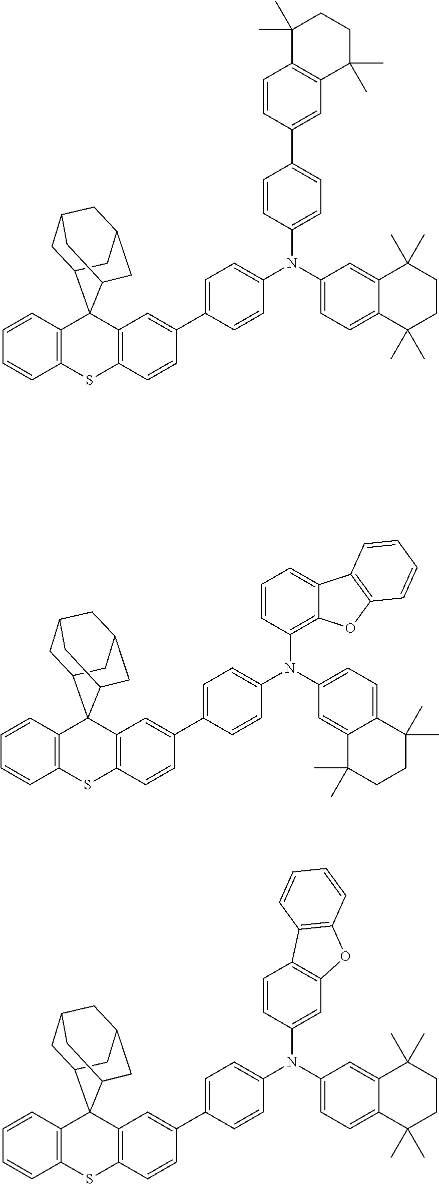 [SMILES: CC1(C)CCC(C)(C)c2cc(-c3ccc(N(c4ccc(-c5ccc6c(c5)C5(c7ccccc7S6)C6CC7CC(C6)CC5C7)cc4)c4ccc5c(c4)C(C)(C)CCC5(C)C)cc3)ccc21 ; CC1(C)CCC(C)(C)c2cc(N(c3ccc(-c4ccc5c(c4)C4(c6ccccc6S5)C5CC6CC(C5)CC4C6)cc3)c3cccc4c3oc3ccccc34)ccc21 ; CC1(C)CCC(C)(C)c2cc(N(c3ccc(-c4ccc5c(c4)C4(c6ccccc6S5)C5CC6CC(C5)CC4C6)cc3)c3ccc4c(c3)oc3ccccc34)ccc21]

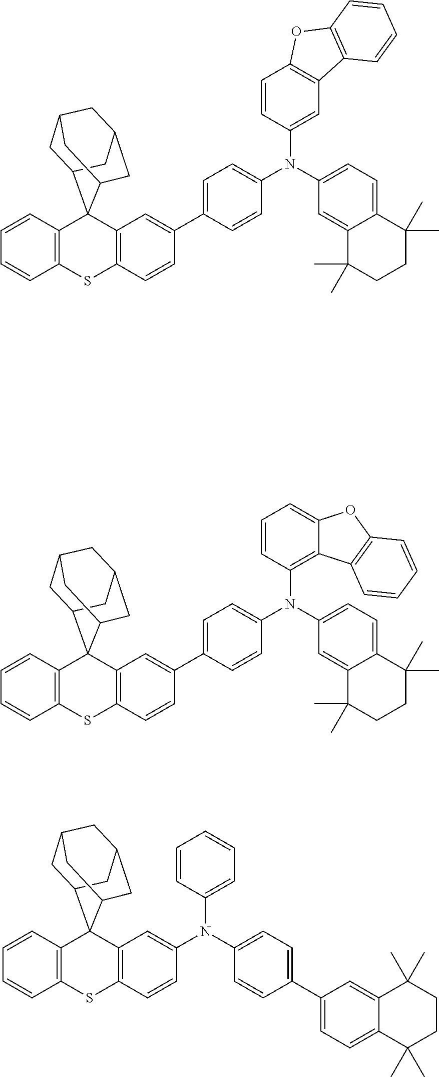 [SMILES: CC1(C)CCC(C)(C)c2cc(N(c3ccc(-c4ccc5c(c4)C4(c6ccccc6S5)C5CC6CC(C5)CC4C6)cc3)c3ccc4oc5ccccc5c4c3)ccc21 ; CC1(C)CCC(C)(C)c2cc(N(c3ccc(-c4ccc5c(c4)C4(c6ccccc6S5)C5CC6CC(C5)CC4C6)cc3)c3cccc4oc5ccccc5c34)ccc21 ; CC1(C)CCC(C)(C)c2cc(-c3ccc(N(c4ccccc4)c4ccc5c(c4)C4(c6ccccc6S5)C5CC6CC(C5)CC4C6)cc3)ccc21]

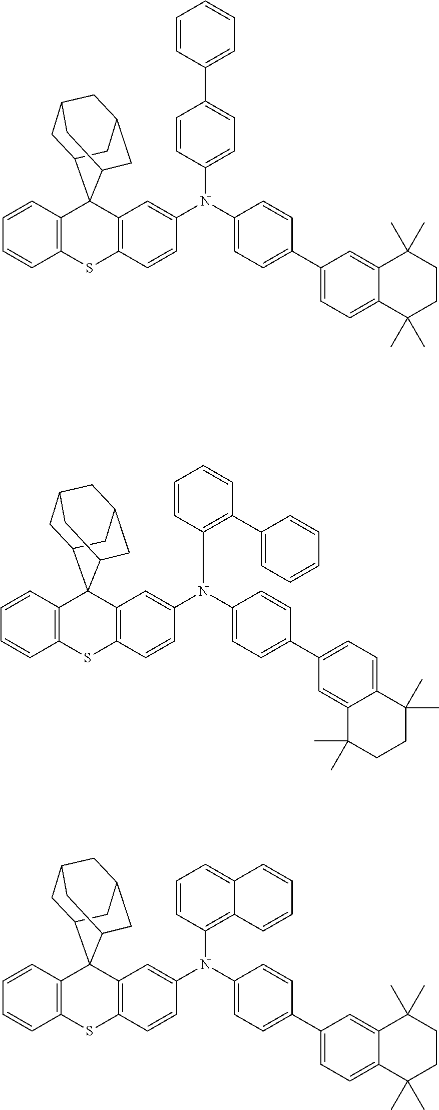 [SMILES: CC1(C)CCC(C)(C)c2cc(-c3ccc(N(c4ccc(-c5ccccc5)cc4)c4ccc5c(c4)C4(c6ccccc6S5)C5CC6CC(C5)CC4C6)cc3)ccc21 ; CC1(C)CCC(C)(C)c2cc(-c3ccc(N(c4ccc5c(c4)C4(c6ccccc6S5)C5CC6CC(C5)CC4C6)c4ccccc4-c4ccccc4)cc3)ccc21 ; CC1(C)CCC(C)(C)c2cc(-c3ccc(N(c4ccc5c(c4)C4(c6ccccc6S5)C5CC6CC(C5)CC4C6)c4cccc5ccccc45)cc3)ccc21]

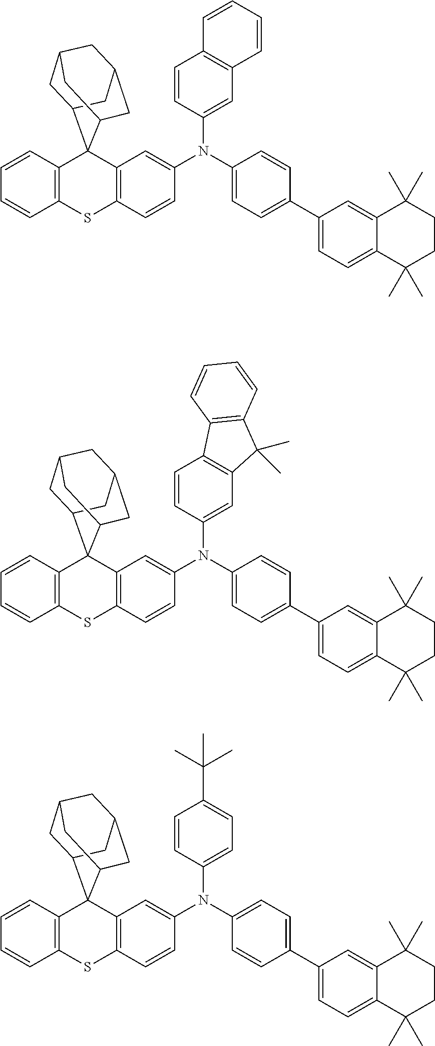 [SMILES: CC1(C)CCC(C)(C)c2cc(-c3ccc(N(c4ccc5c(c4)C4(c6ccccc6S5)C5CC6CC(C5)CC4C6)c4ccc5ccccc5c4)cc3)ccc21 ; CC1(C)CCC(C)(C)c2cc(-c3ccc(N(c4ccc5c(c4)C(C)(C)c4ccccc4-5)c4ccc5c(c4)C4(c6ccccc6S5)C5CC6CC(C5)CC4C6)cc3)ccc21 ; CC(C)(C)c1ccc(N(c2ccc(-c3ccc4c(c3)C(C)(C)CCC4(C)C)cc2)c2ccc3c(c2)C2(c4ccccc4S3)C3CC4CC(C3)CC2C4)cc1]

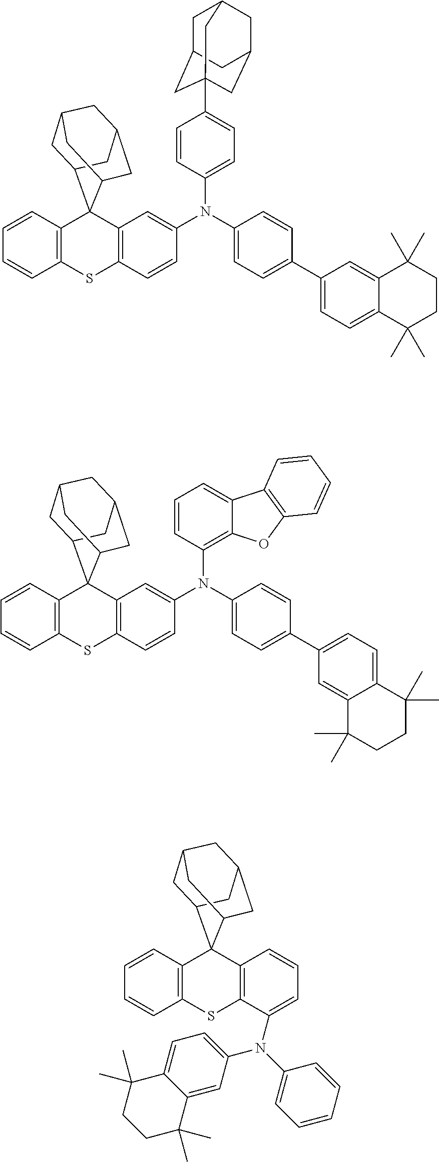

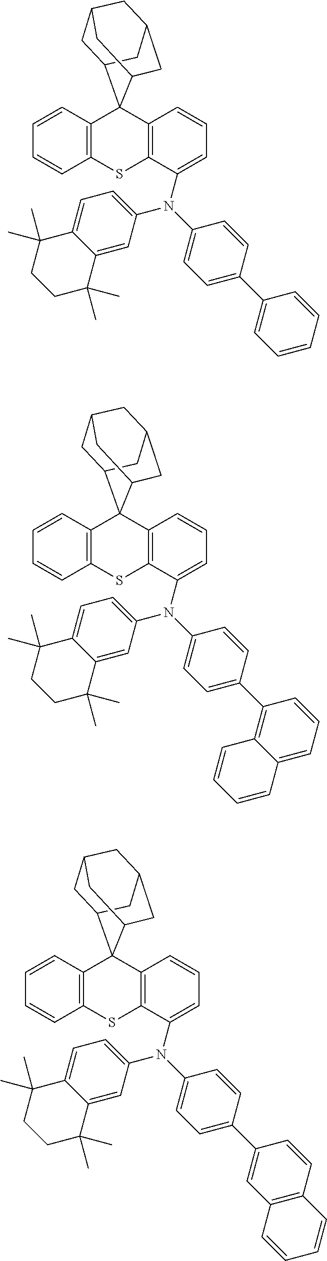 [SMILES: CC1(C)CCC(C)(C)c2cc(N(c3ccc(-c4ccccc4)cc3)c3cccc4c3Sc3ccccc3C43C4CC5CC(C4)CC3C5)ccc21 ; CC1(C)CCC(C)(C)c2cc(N(c3ccc(-c4cccc5ccccc45)cc3)c3cccc4c3Sc3ccccc3C43C4CC5CC(C4)CC3C5)ccc21 ; CC1(C)CCC(C)(C)c2cc(N(c3ccc(-c4ccc5ccccc5c4)cc3)c3cccc4c3Sc3ccccc3C43C4CC5CC(C4)CC3C5)ccc21]

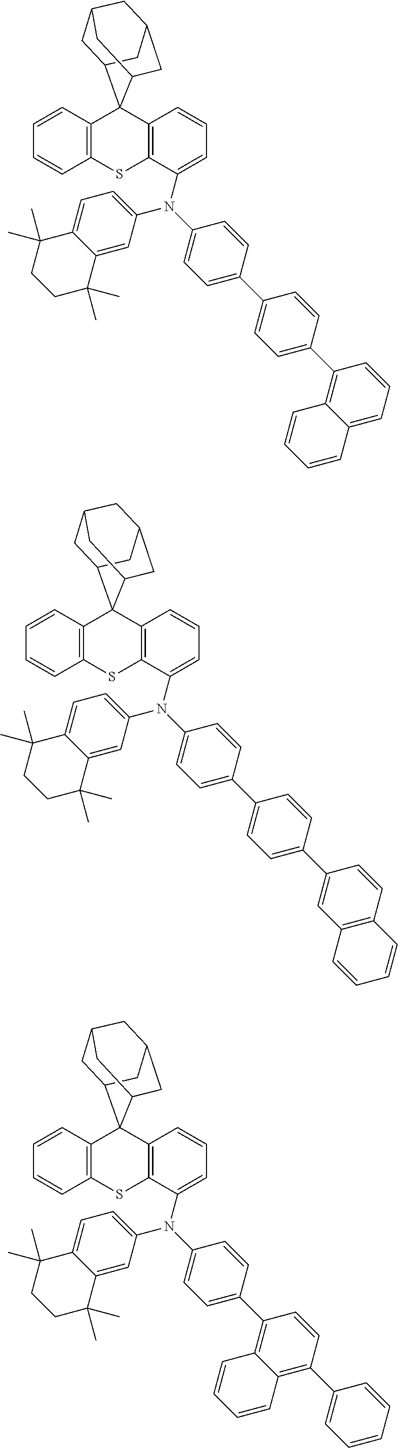 [SMILES: CC1(C)CCC(C)(C)c2cc(N(c3ccc(-c4ccc(-c5cccc6ccccc56)cc4)cc3)c3cccc4c3Sc3ccccc3C43C4CC5CC(C4)CC3C5)ccc21 ; CC1(C)CCC(C)(C)c2cc(N(c3ccc(-c4ccc(-c5ccc6ccccc6c5)cc4)cc3)c3cccc4c3Sc3ccccc3C43C4CC5CC(C4)CC3C5)ccc21 ; CC1(C)CCC(C)(C)c2cc(N(c3ccc(-c4ccc(-c5ccccc5)c5ccccc45)cc3)c3cccc4c3Sc3ccccc3C43C4CC5CC(C4)CC3C5)ccc21]

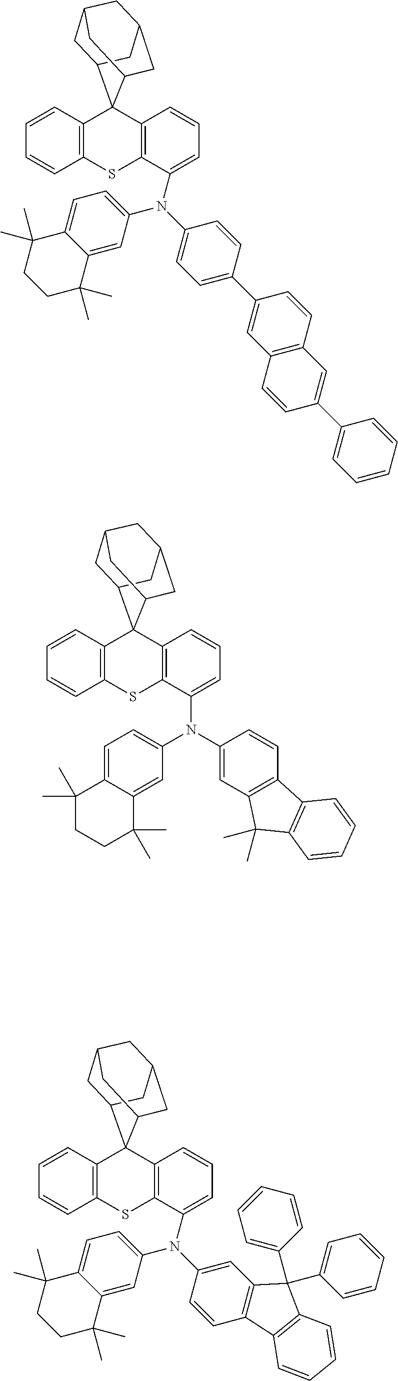 [SMILES: CC1(C)CCC(C)(C)c2cc(N(c3ccc(-c4ccc5cc(-c6ccccc6)ccc5c4)cc3)c3cccc4c3Sc3ccccc3C43C4CC5CC(C4)CC3C5)ccc21 ; CC1(C)CCC(C)(C)c2cc(N(c3ccc4c(c3)C(C)(C)c3ccccc3-4)c3cccc4c3Sc3ccccc3C43C4CC5CC(C4)CC3C5)ccc21 ; CC1(C)CCC(C)(C)c2cc(N(c3ccc4c(c3)C(c3ccccc3)(c3ccccc3)c3ccccc3-4)c3cccc4c3Sc3ccccc3C43C4CC5CC(C4)CC3C5)ccc21]

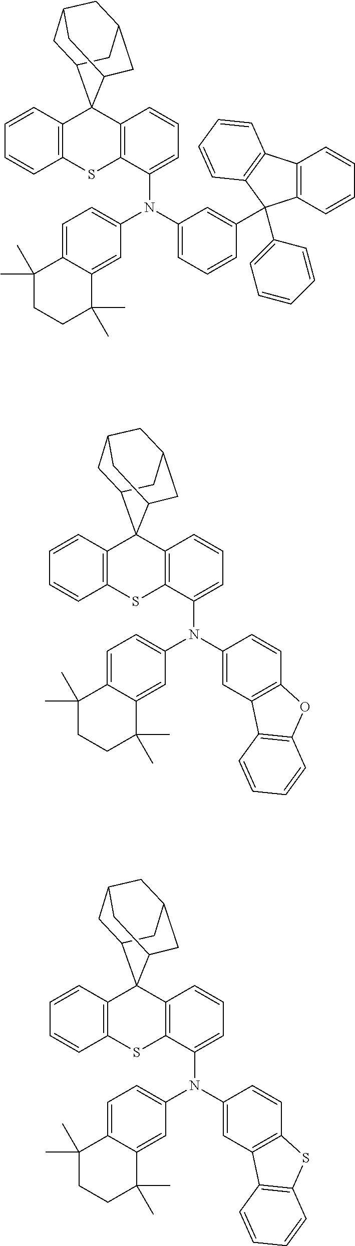 [SMILES: CC1(C)CCC(C)(C)c2cc(N(c3cccc(C4(c5ccccc5)c5ccccc5-c5ccccc54)c3)c3cccc4c3Sc3ccccc3C43C4CC5CC(C4)CC3C5)ccc21 ; CC1(C)CCC(C)(C)c2cc(N(c3ccc4oc5ccccc5c4c3)c3cccc4c3Sc3ccccc3C43C4CC5CC(C4)CC3C5)ccc21 ; CC1(C)CCC(C)(C)c2cc(N(c3ccc4sc5ccccc5c4c3)c3cccc4c3Sc3ccccc3C43C4CC5CC(C4)CC3C5)ccc21]

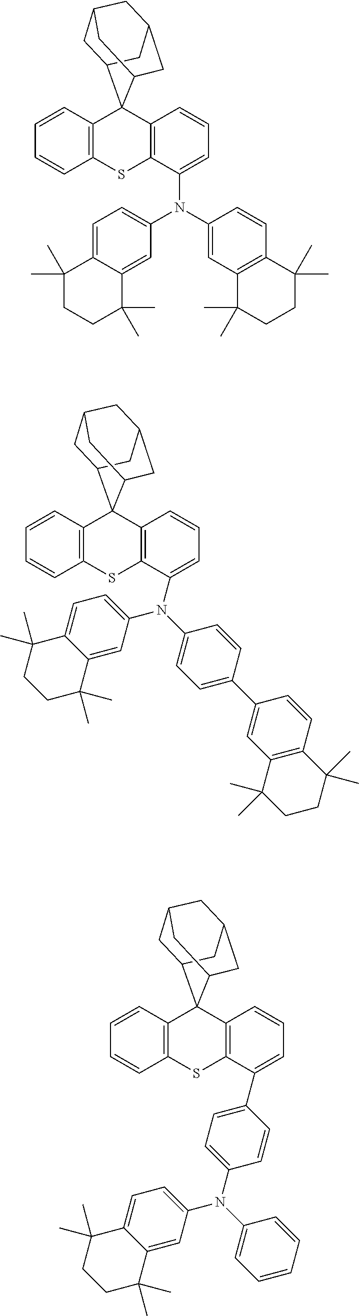

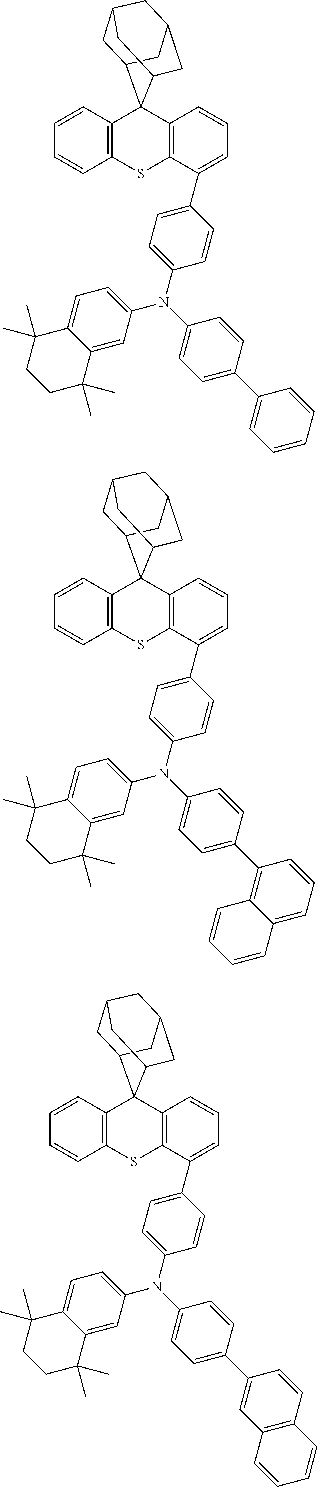 [SMILES: CC1(C)CCC(C)(C)c2cc(N(c3ccc(-c4ccccc4)cc3)c3ccc(-c4cccc5c4Sc4ccccc4C54C5CC6CC(C5)CC4C6)cc3)ccc21 ; CC1(C)CCC(C)(C)c2cc(N(c3ccc(-c4cccc5c4Sc4ccccc4C54C5CC6CC(C5)CC4C6)cc3)c3ccc(-c4cccc5ccccc45)cc3)ccc21 ; CC1(C)CCC(C)(C)c2cc(N(c3ccc(-c4ccc5ccccc5c4)cc3)c3ccc(-c4cccc5c4Sc4ccccc4C54C5CC6CC(C5)CC4C6)cc3)ccc21]

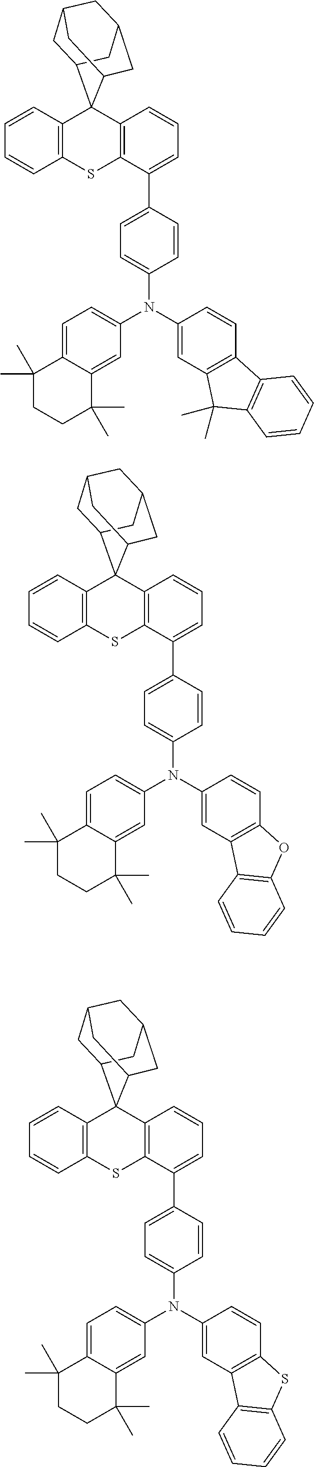

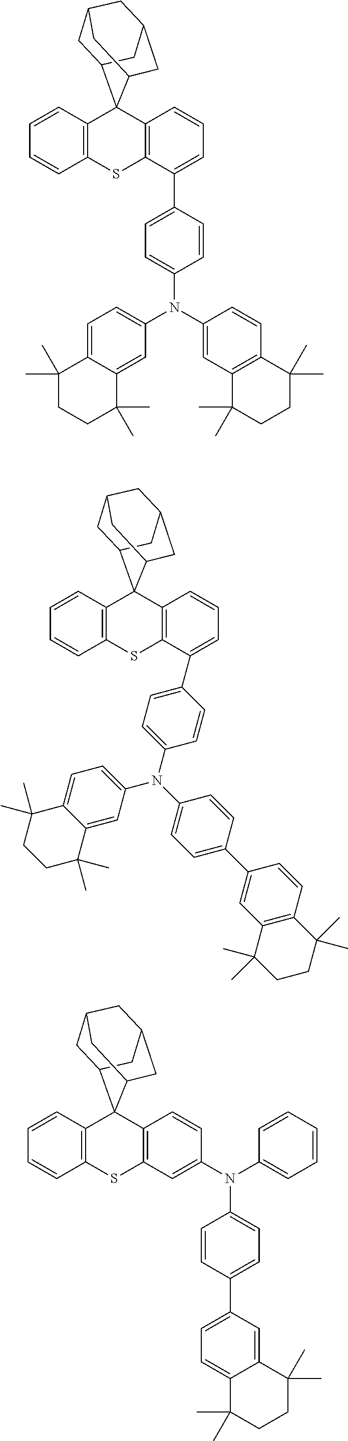

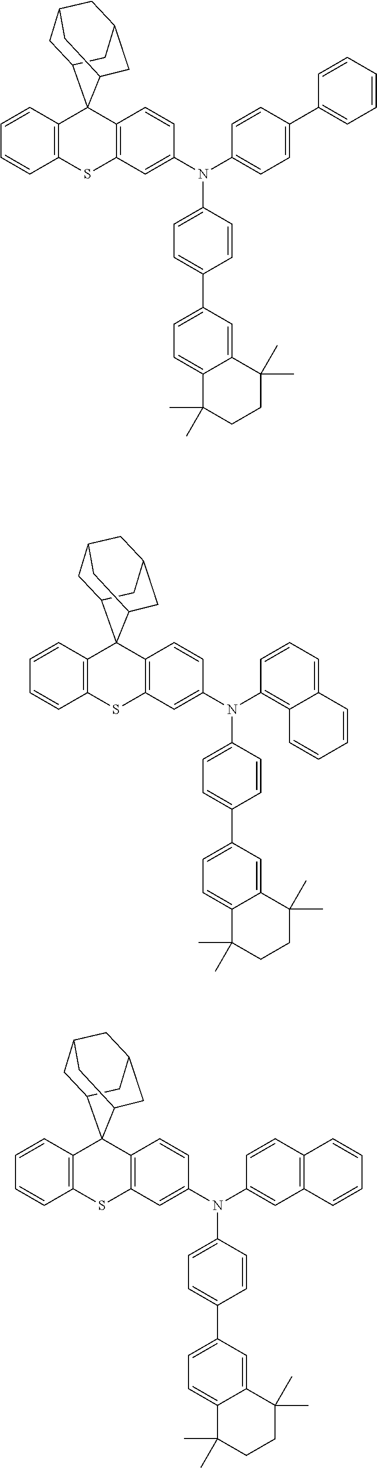 [SMILES: CC1(C)CCC(C)(C)c2cc(-c3ccc(N(c4ccc(-c5ccccc5)cc4)c4ccc5c(c4)Sc4ccccc4C54C5CC6CC(C5)CC4C6)cc3)ccc21 ; CC1(C)CCC(C)(C)c2cc(-c3ccc(N(c4ccc5c(c4)Sc4ccccc4C54C5CC6CC(C5)CC4C6)c4cccc5ccccc45)cc3)ccc21 ; CC1(C)CCC(C)(C)c2cc(-c3ccc(N(c4ccc5c(c4)Sc4ccccc4C54C5CC6CC(C5)CC4C6)c4ccc5ccccc5c4)cc3)ccc21]

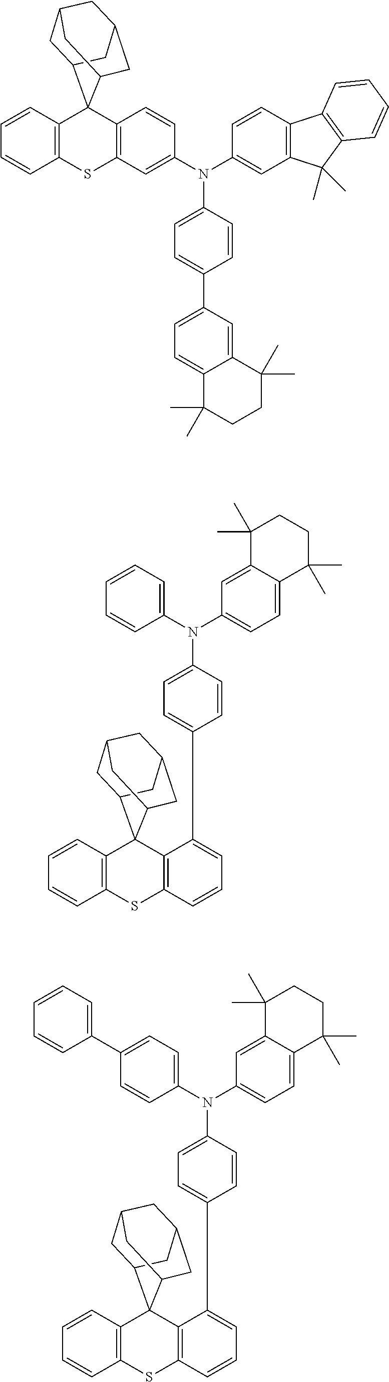 [SMILES: CC1(C)CCC(C)(C)c2cc(-c3ccc(N(c4ccc5c(c4)Sc4ccccc4C54C5CC6CC(C5)CC4C6)c4ccc5c(c4)C(C)(C)c4ccccc4-5)cc3)ccc21 ; CC1(C)CCC(C)(C)c2cc(N(c3ccccc3)c3ccc(-c4cccc5c4C4(c6ccccc6S5)C5CC6CC(C5)CC4C6)cc3)ccc21 ; CC1(C)CCC(C)(C)c2cc(N(c3ccc(-c4ccccc4)cc3)c3ccc(-c4cccc5c4C4(c6ccccc6S5)C5CC6CC(C5)CC4C6)cc3)ccc21]

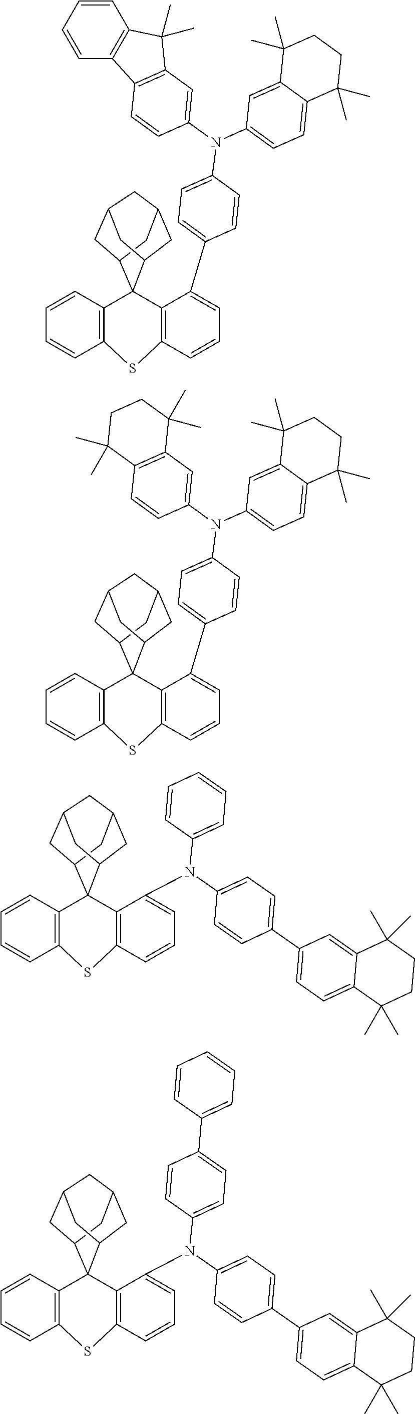

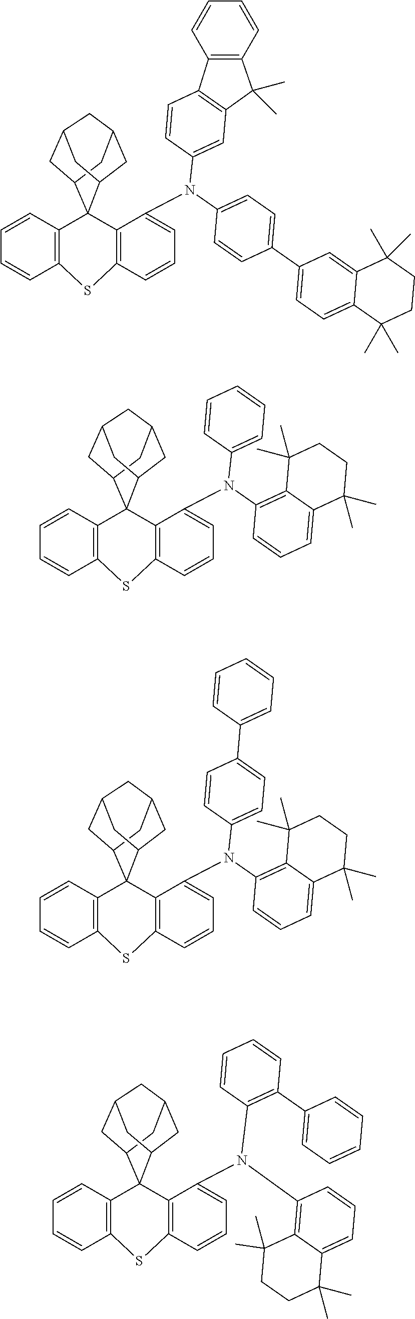 [SMILES: CC1(C)CCC(C)(C)c2cc(-c3ccc(N(c4ccc5c(c4)C(C)(C)c4ccccc4-5)c4cccc5c4C4(c6ccccc6S5)C5CC6CC(C5)CC4C6)cc3)ccc21 ; CC1(C)CCC(C)(C)c2c(N(c3ccccc3)c3cccc4c3C3(c5ccccc5S4)C4CC5CC(C4)CC3C5)cccc21 ; CC1(C)CCC(C)(C)c2c(N(c3ccc(-c4ccccc4)cc3)c3cccc4c3C3(c5ccccc5S4)C4CC5CC(C4)CC3C5)cccc21 ; CC1(C)CCC(C)(C)c2c(N(c3ccccc3-c3ccccc3)c3cccc4c3C3(c5ccccc5S4)C4CC5CC(C4)CC3C5)cccc21]

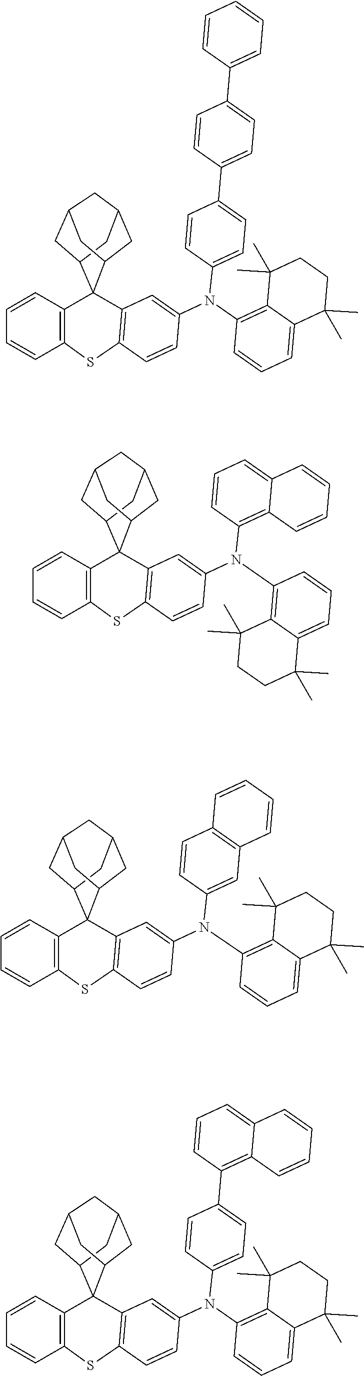 [SMILES: CC1(C)CCC(C)(C)c2c(N(c3ccc(-c4ccc(-c5ccccc5)cc4)cc3)c3ccc4c(c3)C3(c5ccccc5S4)C4CC5CC(C4)CC3C5)cccc21 ; CC1(C)CCC(C)(C)c2c(N(c3ccc4c(c3)C3(c5ccccc5S4)C4CC5CC(C4)CC3C5)c3cccc4ccccc34)cccc21 ; CC1(C)CCC(C)(C)c2c(N(c3ccc4c(c3)C3(c5ccccc5S4)C4CC5CC(C4)CC3C5)c3ccc4ccccc4c3)cccc21 ; CC1(C)CCC(C)(C)c2c(N(c3ccc(-c4cccc5ccccc45)cc3)c3ccc4c(c3)C3(c5ccccc5S4)C4CC5CC(C4)CC3C5)cccc21]

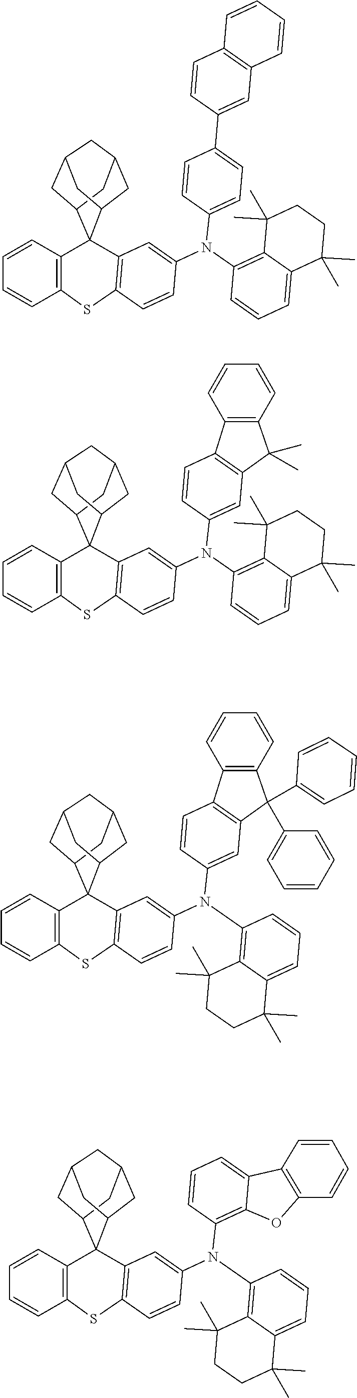

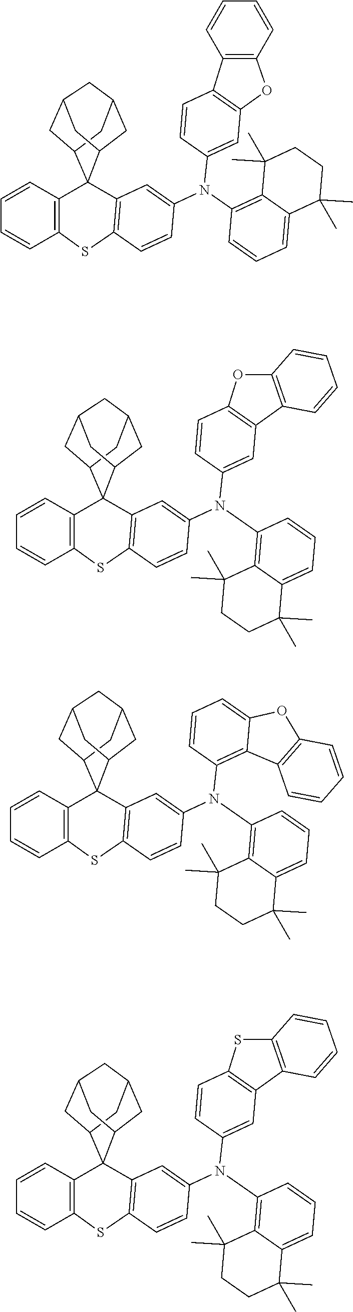 [SMILES: CC1(C)CCC(C)(C)c2c(N(c3ccc4c(c3)C3(c5ccccc5S4)C4CC5CC(C4)CC3C5)c3ccc4c(c3)oc3ccccc34)cccc21 ; CC1(C)CCC(C)(C)c2c(N(c3ccc4c(c3)C3(c5ccccc5S4)C4CC5CC(C4)CC3C5)c3ccc4oc5ccccc5c4c3)cccc21 ; CC1(C)CCC(C)(C)c2c(N(c3ccc4c(c3)C3(c5ccccc5S4)C4CC5CC(C4)CC3C5)c3cccc4oc5ccccc5c34)cccc21 ; CC1(C)CCC(C)(C)c2c(N(c3ccc4c(c3)C3(c5ccccc5S4)C4CC5CC(C4)CC3C5)c3ccc4sc5ccccc5c4c3)cccc21]

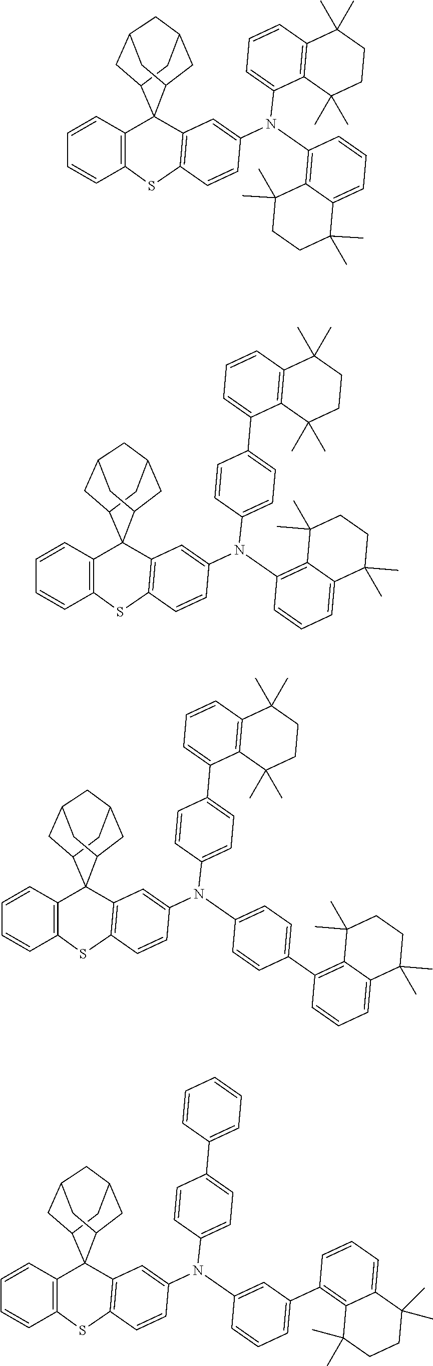 [SMILES: CC1(C)CCC(C)(C)c2c(N(c3ccc4c(c3)C3(c5ccccc5S4)C4CC5CC(C4)CC3C5)c3cccc4c3C(C)(C)CCC4(C)C)cccc21 ; CC1(C)CCC(C)(C)c2c(-c3ccc(N(c4ccc5c(c4)C4(c6ccccc6S5)C5CC6CC(C5)CC4C6)c4cccc5c4C(C)(C)CCC5(C)C)cc3)cccc21 ; CC1(C)CCC(C)(C)c2c(-c3ccc(N(c4ccc(-c5cccc6c5C(C)(C)CCC6(C)C)cc4)c4ccc5c(c4)C4(c6ccccc6S5)C5CC6CC(C5)CC4C6)cc3)cccc21 ; CC1(C)CCC(C)(C)c2c(-c3cccc(N(c4ccc(-c5ccccc5)cc4)c4ccc5c(c4)C4(c6ccccc6S5)C5CC6CC(C5)CC4C6)c3)cccc21]

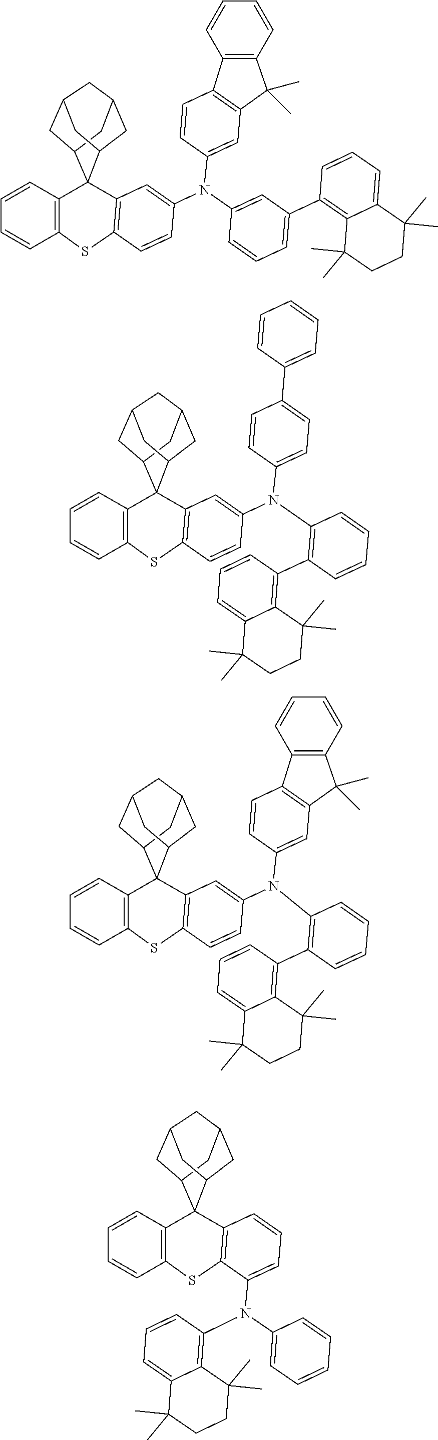

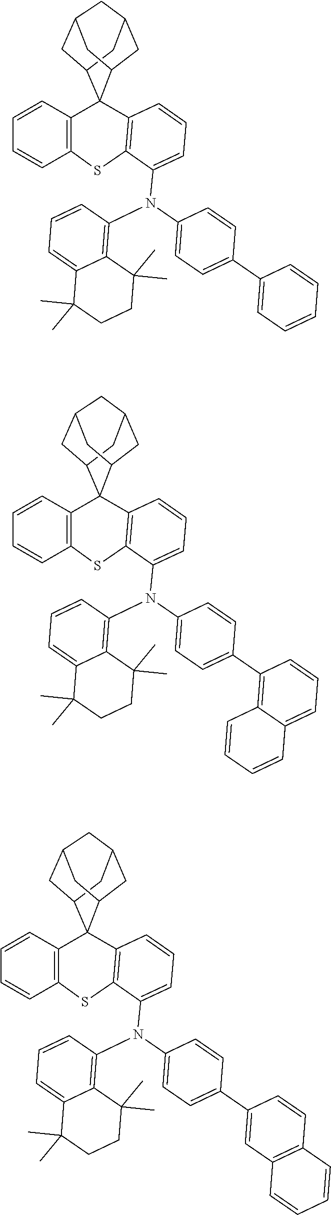 [SMILES: CC1(C)CCC(C)(C)c2c(N(c3ccc(-c4ccccc4)cc3)c3cccc4c3Sc3ccccc3C43C4CC5CC(C4)CC3C5)cccc21 ; CC1(C)CCC(C)(C)c2c(N(c3ccc(-c4cccc5ccccc45)cc3)c3cccc4c3Sc3ccccc3C43C4CC5CC(C4)CC3C5)cccc21 ; CC1(C)CCC(C)(C)c2c(N(c3ccc(-c4ccc5ccccc5c4)cc3)c3cccc4c3Sc3ccccc3C43C4CC5CC(C4)CC3C5)cccc21]

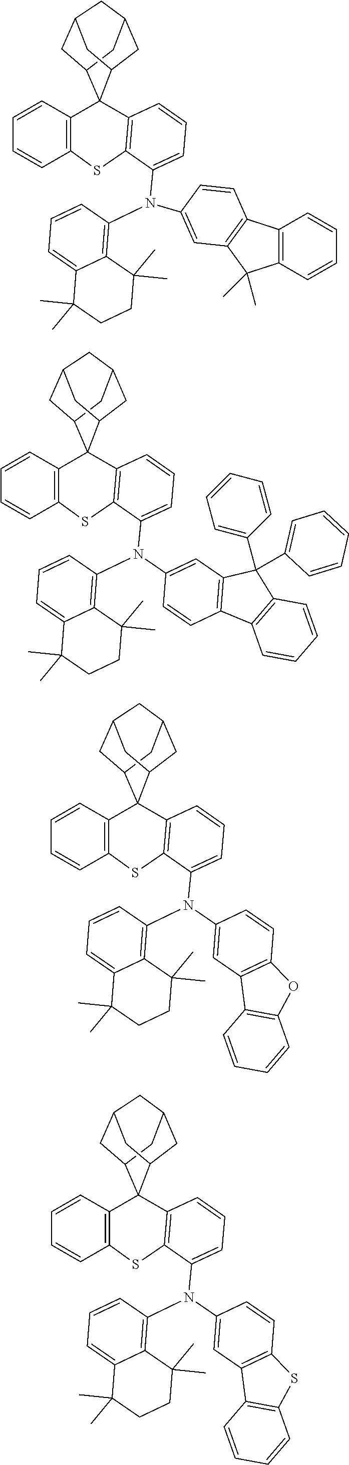

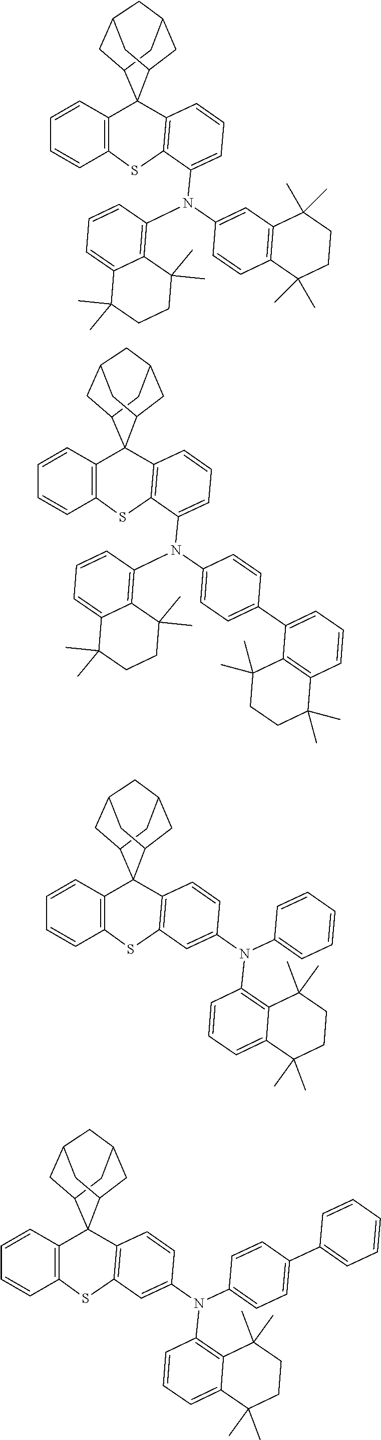

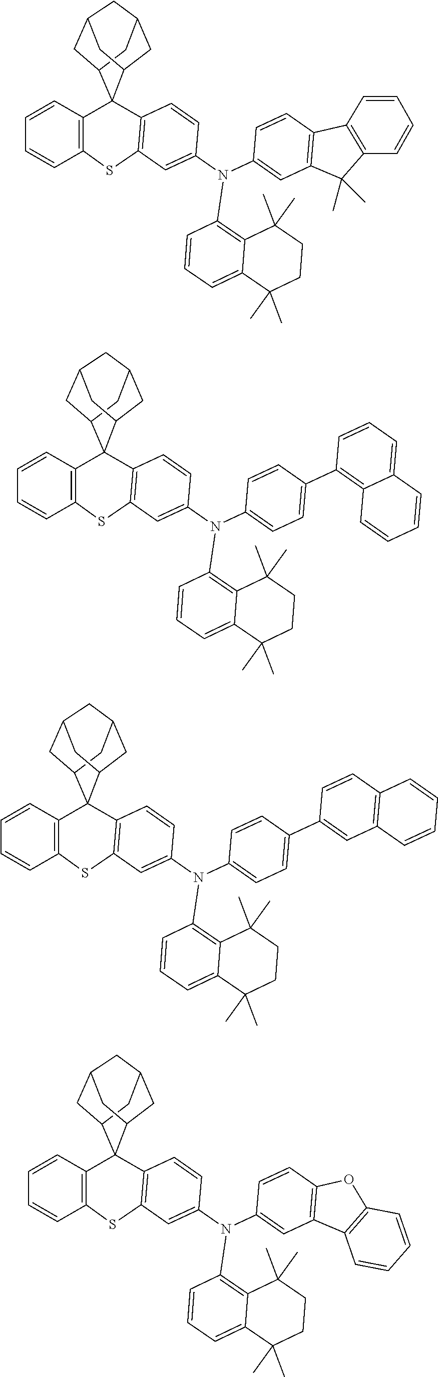 [SMILES: CC1(C)CCC(C)(C)c2c(N(c3ccc4c(c3)Sc3ccccc3C43C4CC5CC(C4)CC3C5)c3ccc4c(c3)C(C)(C)c3ccccc3-4)cccc21 ; CC1(C)CCC(C)(C)c2c(N(c3ccc(-c4cccc5ccccc45)cc3)c3ccc4c(c3)Sc3ccccc3C43C4CC5CC(C4)CC3C5)cccc21 ; CC1(C)CCC(C)(C)c2c(N(c3ccc(-c4ccc5ccccc5c4)cc3)c3ccc4c(c3)Sc3ccccc3C43C4CC5CC(C4)CC3C5)cccc21 ; CC1(C)CCC(C)(C)c2c(N(c3ccc4c(c3)Sc3ccccc3C43C4CC5CC(C4)CC3C5)c3ccc4oc5ccccc5c4c3)cccc21]

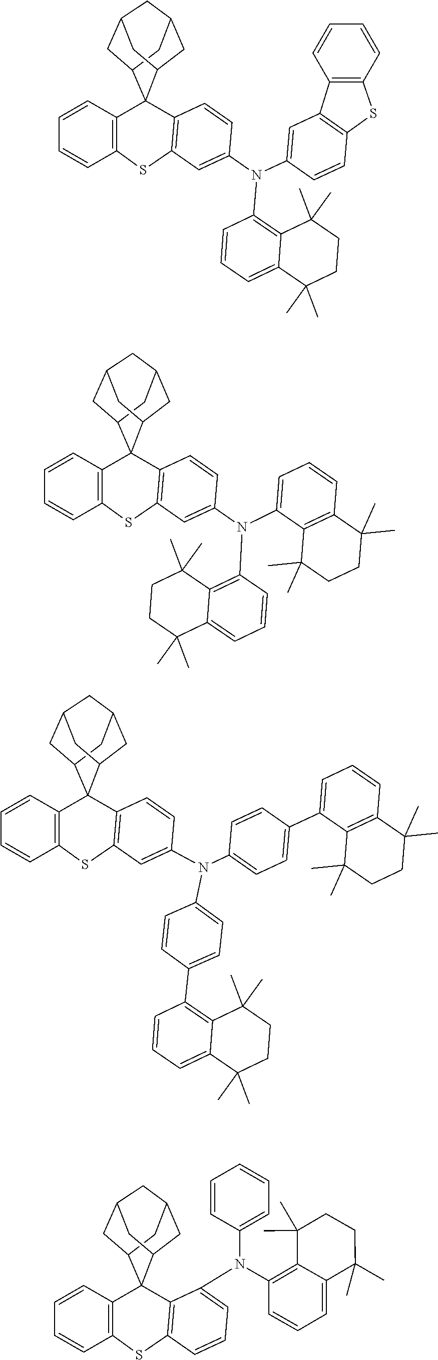 [SMILES: CC1(C)CCC(C)(C)c2c(N(c3ccc4c(c3)Sc3ccccc3C43C4CC5CC(C4)CC3C5)c3ccc4sc5ccccc5c4c3)cccc21 ; CC1(C)CCC(C)(C)c2c(N(c3ccc4c(c3)Sc3ccccc3C43C4CC5CC(C4)CC3C5)c3cccc4c3C(C)(C)CCC4(C)C)cccc21 ; CC1(C)CCC(C)(C)c2c(-c3ccc(N(c4ccc(-c5cccc6c5C(C)(C)CCC6(C)C)cc4)c4ccc5c(c4)Sc4ccccc4C54C5CC6CC(C5)CC4C6)cc3)cccc21 ; CC1(C)CCC(C)(C)c2c(N(c3ccccc3)c3cccc4c3C3(c5ccccc5S4)C4CC5CC(C4)CC3C5)cccc21]

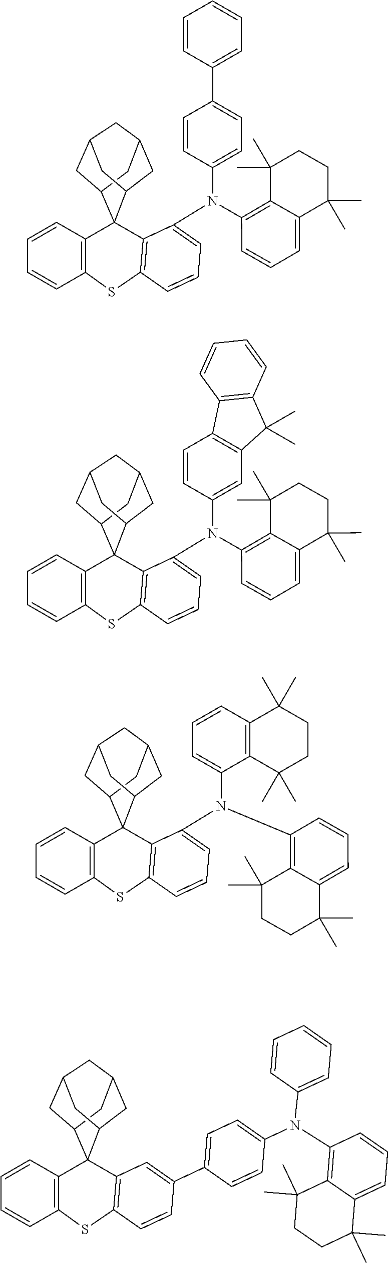 [SMILES: CC1(C)CCC(C)(C)c2c(N(c3ccc(-c4ccccc4)cc3)c3cccc4c3C3(c5ccccc5S4)C4CC5CC(C4)CC3C5)cccc21 ; CC1(C)CCC(C)(C)c2c(N(c3ccc4c(c3)C(C)(C)c3ccccc3-4)c3cccc4c3C3(c5ccccc5S4)C4CC5CC(C4)CC3C5)cccc21 ; CC1(C)CCC(C)(C)c2c(N(c3cccc4c3C(C)(C)CCC4(C)C)c3cccc4c3C3(c5ccccc5S4)C4CC5CC(C4)CC3C5)cccc21 ; CC1(C)CCC(C)(C)c2c(N(c3ccccc3)c3ccc(-c4ccc5c(c4)C4(c6ccccc6S5)C5CC6CC(C5)CC4C6)cc3)cccc21]

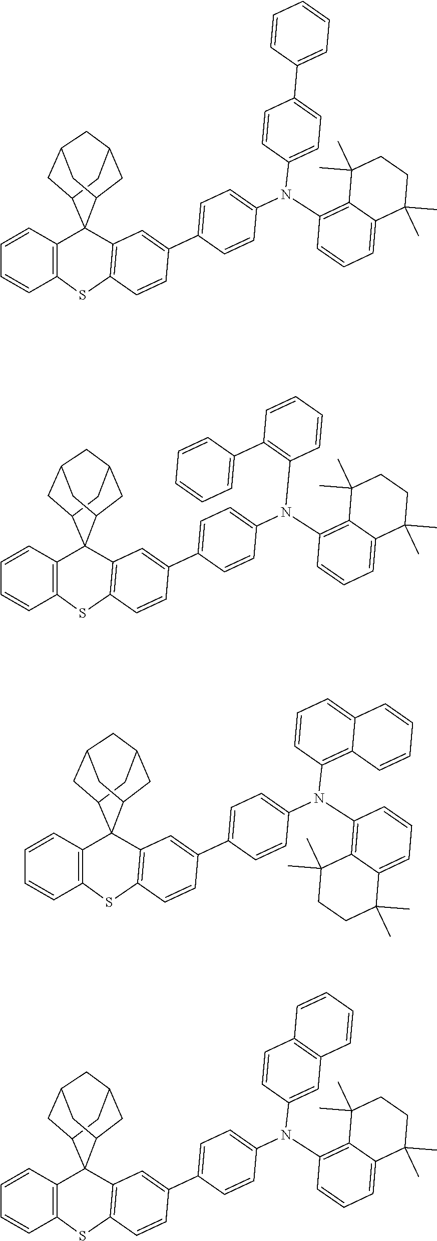 [SMILES: CC1(C)CCC(C)(C)c2c(N(c3ccc(-c4ccccc4)cc3)c3ccc(-c4ccc5c(c4)C4(c6ccccc6S5)C5CC6CC(C5)CC4C6)cc3)cccc21 ; CC1(C)CCC(C)(C)c2c(N(c3ccc(-c4ccc5c(c4)C4(c6ccccc6S5)C5CC6CC(C5)CC4C6)cc3)c3ccccc3-c3ccccc3)cccc21 ; CC1(C)CCC(C)(C)c2c(N(c3ccc(-c4ccc5c(c4)C4(c6ccccc6S5)C5CC6CC(C5)CC4C6)cc3)c3cccc4ccccc34)cccc21 ; CC1(C)CCC(C)(C)c2c(N(c3ccc(-c4ccc5c(c4)C4(c6ccccc6S5)C5CC6CC(C5)CC4C6)cc3)c3ccc4ccccc4c3)cccc21]

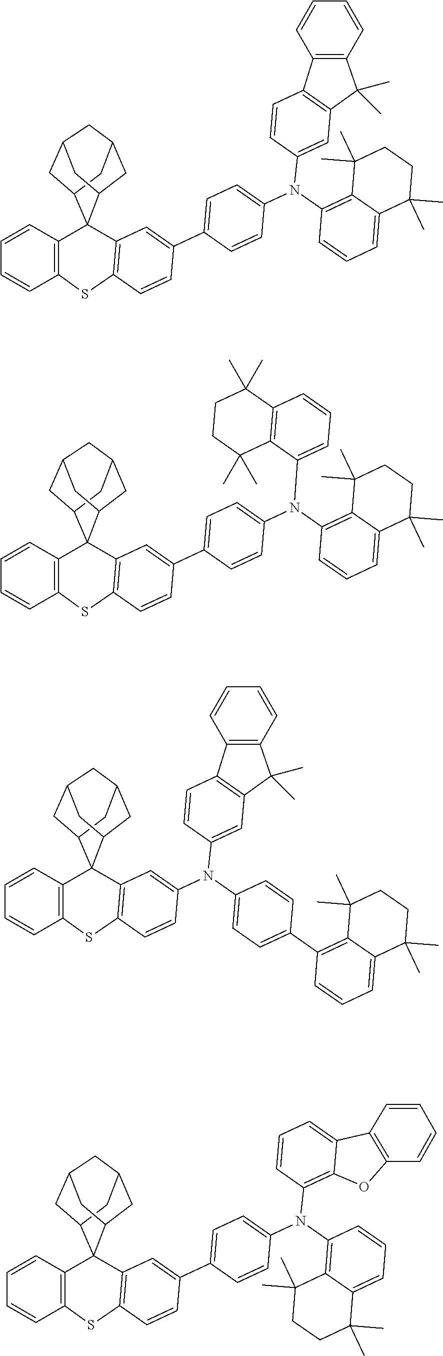 [SMILES: CC1(C)CCC(C)(C)c2c(N(c3ccc(-c4ccc5c(c4)C4(c6ccccc6S5)C5CC6CC(C5)CC4C6)cc3)c3ccc4c(c3)C(C)(C)c3ccccc3-4)cccc21 ; CC1(C)CCC(C)(C)c2c(N(c3ccc(-c4ccc5c(c4)C4(c6ccccc6S5)C5CC6CC(C5)CC4C6)cc3)c3cccc4c3C(C)(C)CCC4(C)C)cccc21 ; CC1(C)CCC(C)(C)c2c(-c3ccc(N(c4ccc5c(c4)C(C)(C)c4ccccc4-5)c4ccc5c(c4)C4(c6ccccc6S5)C5CC6CC(C5)CC4C6)cc3)cccc21 ; CC1(C)CCC(C)(C)c2c(N(c3ccc(-c4ccc5c(c4)C4(c6ccccc6S5)C5CC6CC(C5)CC4C6)cc3)c3cccc4c3oc3ccccc34)cccc21]

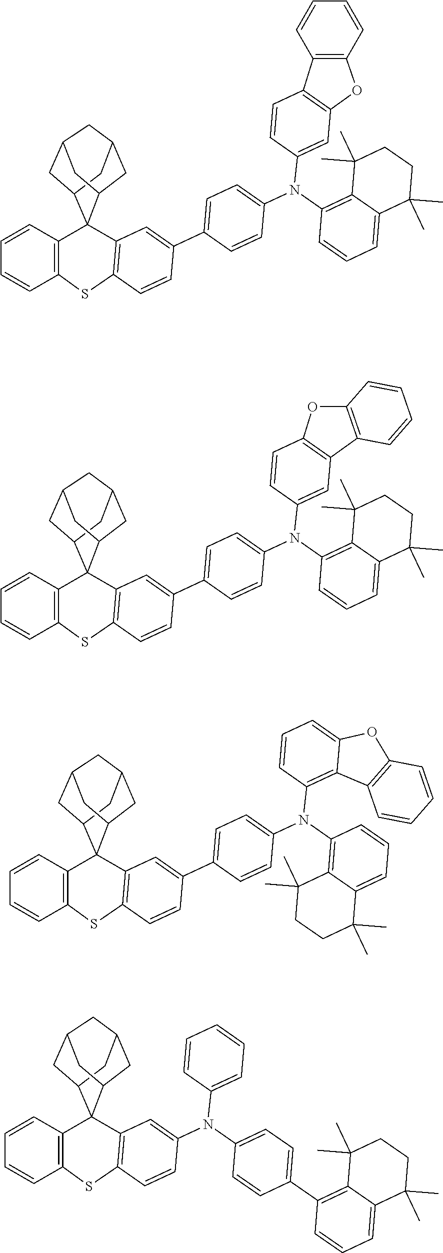

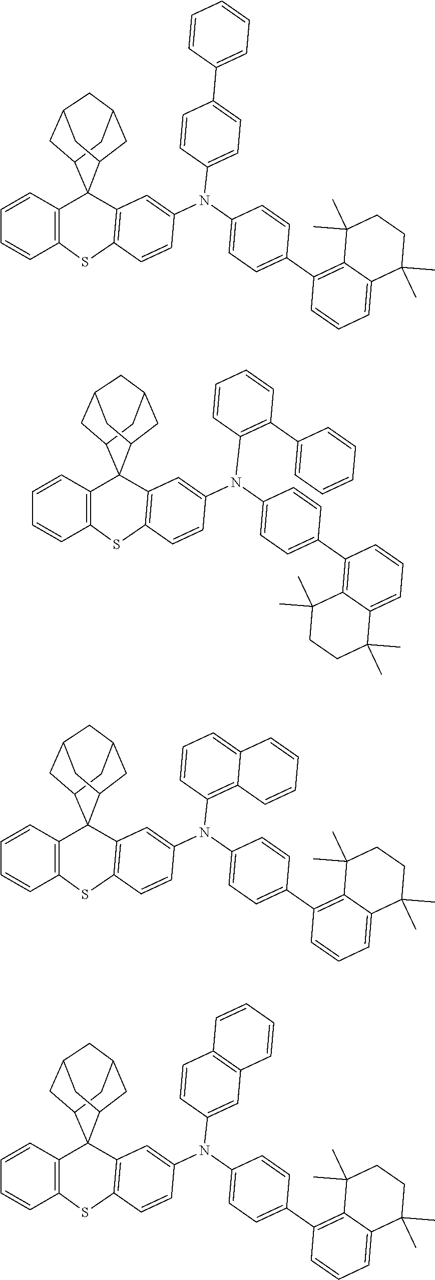 [SMILES: CC1(C)CCC(C)(C)c2c(-c3ccc(N(c4ccc(-c5ccccc5)cc4)c4ccc5c(c4)C4(c6ccccc6S5)C5CC6CC(C5)CC4C6)cc3)cccc21 ; CC1(C)CCC(C)(C)c2c(-c3ccc(N(c4ccc5c(c4)C4(c6ccccc6S5)C5CC6CC(C5)CC4C6)c4ccccc4-c4ccccc4)cc3)cccc21 ; CC1(C)CCC(C)(C)c2c(-c3ccc(N(c4ccc5c(c4)C4(c6ccccc6S5)C5CC6CC(C5)CC4C6)c4cccc5ccccc45)cc3)cccc21 ; CC1(C)CCC(C)(C)c2c(-c3ccc(N(c4ccc5c(c4)C4(c6ccccc6S5)C5CC6CC(C5)CC4C6)c4ccc5ccccc5c4)cc3)cccc21]

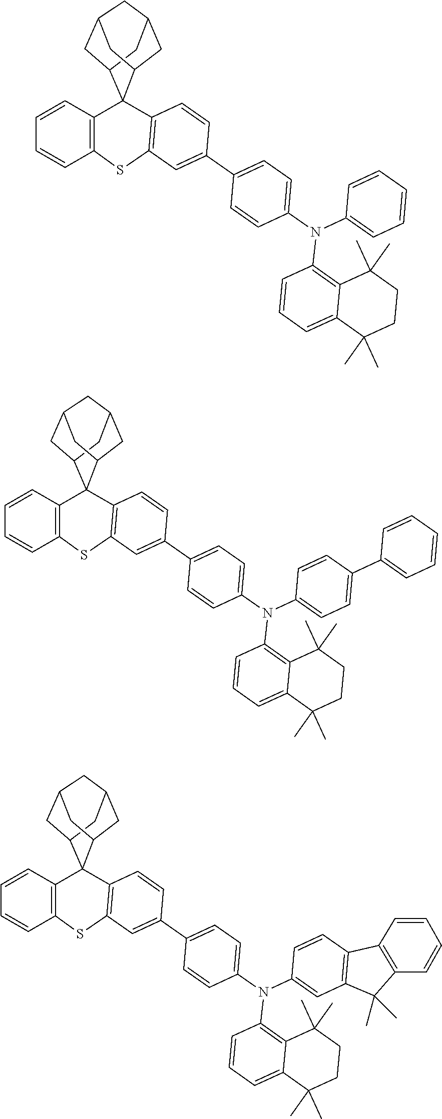 [SMILES: CC1(C)CCC(C)(C)c2c(N(c3ccccc3)c3ccc(-c4ccc5c(c4)Sc4ccccc4C54C5CC6CC(C5)CC4C6)cc3)cccc21 ; CC1(C)CCC(C)(C)c2c(N(c3ccc(-c4ccccc4)cc3)c3ccc(-c4ccc5c(c4)Sc4ccccc4C54C5CC6CC(C5)CC4C6)cc3)cccc21 ; CC1(C)CCC(C)(C)c2c(N(c3ccc(-c4ccc5c(c4)Sc4ccccc4C54C5CC6CC(C5)CC4C6)cc3)c3ccc4c(c3)C(C)(C)c3ccccc3-4)cccc21]

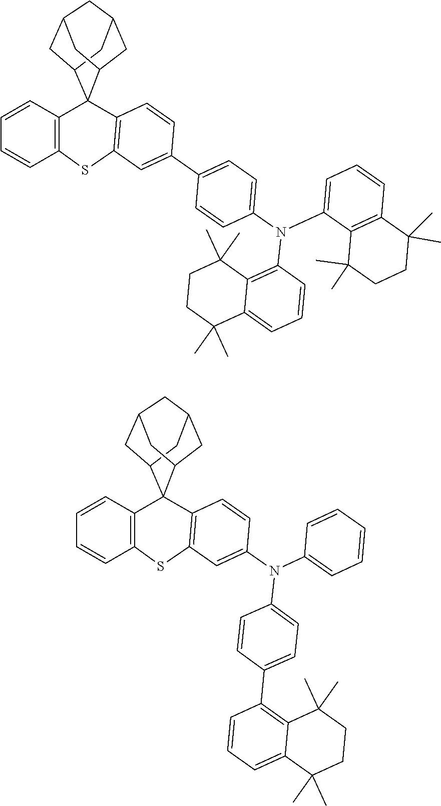 [SMILES: CC1(C)CCC(C)(C)c2c(N(c3ccc(-c4ccc5c(c4)Sc4ccccc4C54C5CC6CC(C5)CC4C6)cc3)c3cccc4c3C(C)(C)CCC4(C)C)cccc21 ; CC1(C)CCC(C)(C)c2c(-c3ccc(N(c4ccccc4)c4ccc5c(c4)Sc4ccccc4C54C5CC6CC(C5)CC4C6)cc3)cccc21]

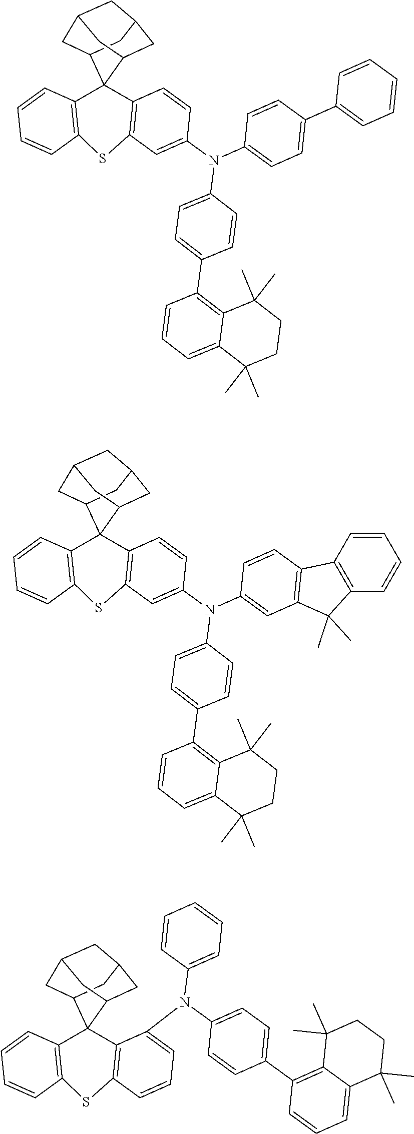

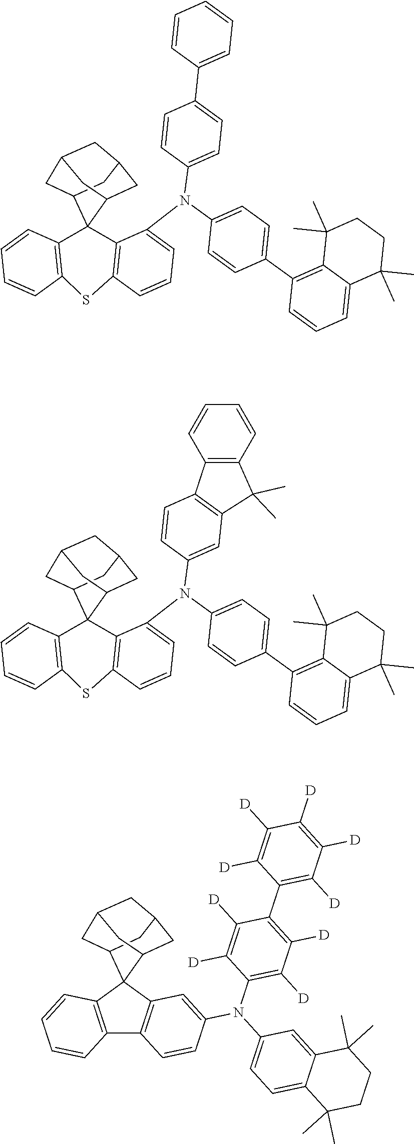

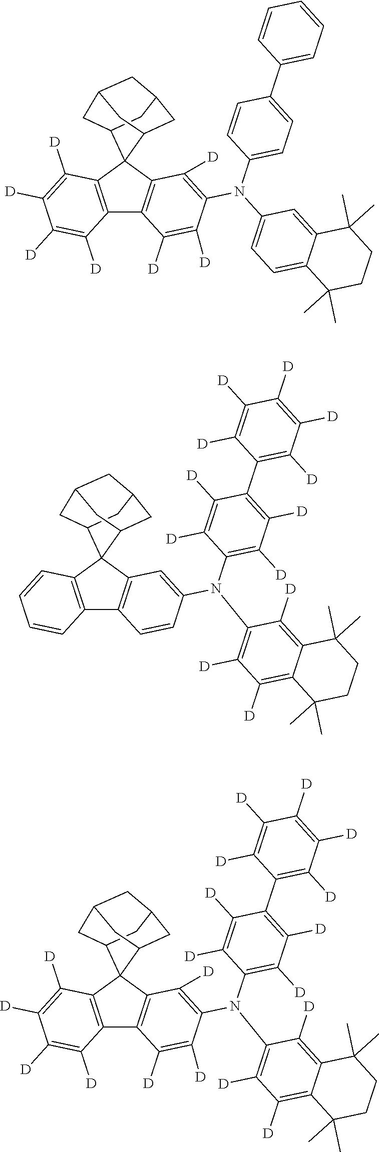 [SMILES: [2H]c1c([2H])c([2H])c2c(c1[2H])-c1c([2H])c([2H])c(N(c3ccc(-c4ccccc4)cc3)c3ccc4c(c3)C(C)(C)CCC4(C)C)c([2H])c1C21C2CC3CC(C2)CC1C3 ; [2H]c1c([2H])c([2H])c(-c2c([2H])c([2H])c(N(c3ccc4c(c3)C3(c5ccccc5-4)C4CC5CC(C4)CC3C5)c3c([2H])c([2H])c4c(c3[2H])C(C)(C)CCC4(C)C)c([2H])c2[2H])c([2H])c1[2H] ; [2H]c1c([2H])c([2H])c(-c2c([2H])c([2H])c(N(c3c([2H])c([2H])c4c(c3[2H])C(C)(C)CCC4(C)C)c3c([2H])c([2H])c4c(c3[2H])C3(c5c([2H])c([2H])c([2H])c([2H])c5-4)C4CC5CC(C4)CC3C5)c([2H])c2[2H])c([2H])c1[2H]]

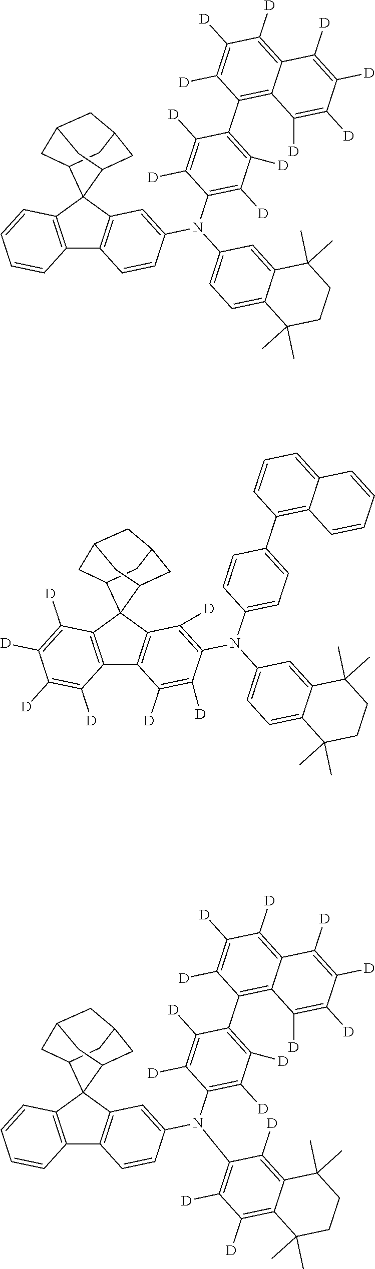 [SMILES: [2H]c1c([2H])c(N(c2ccc3c(c2)C(C)(C)CCC3(C)C)c2ccc3c(c2)C2(c4ccccc4-3)C3CC4CC(C3)CC2C4)c([2H])c([2H])c1-c1c([2H])c([2H])c([2H])c2c([2H])c([2H])c([2H])c([2H])c12 ; [2H]c1c([2H])c([2H])c2c(c1[2H])-c1c([2H])c([2H])c(N(c3ccc(-c4cccc5ccccc45)cc3)c3ccc4c(c3)C(C)(C)CCC4(C)C)c([2H])c1C21C2CC3CC(C2)CC1C3 ; [2H]c1c([2H])c(N(c2ccc3c(c2)C2(c4ccccc4-3)C3CC4CC(C3)CC2C4)c2c([2H])c([2H])c3c(c2[2H])C(C)(C)CCC3(C)C)c([2H])c([2H])c1-c1c([2H])c([2H])c([2H])c2c([2H])c([2H])c([2H])c([2H])c12]

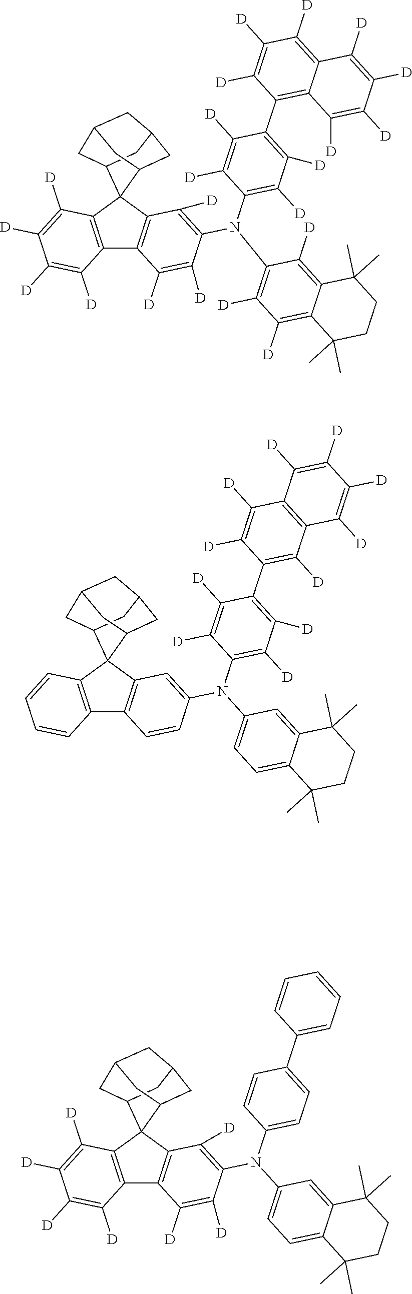

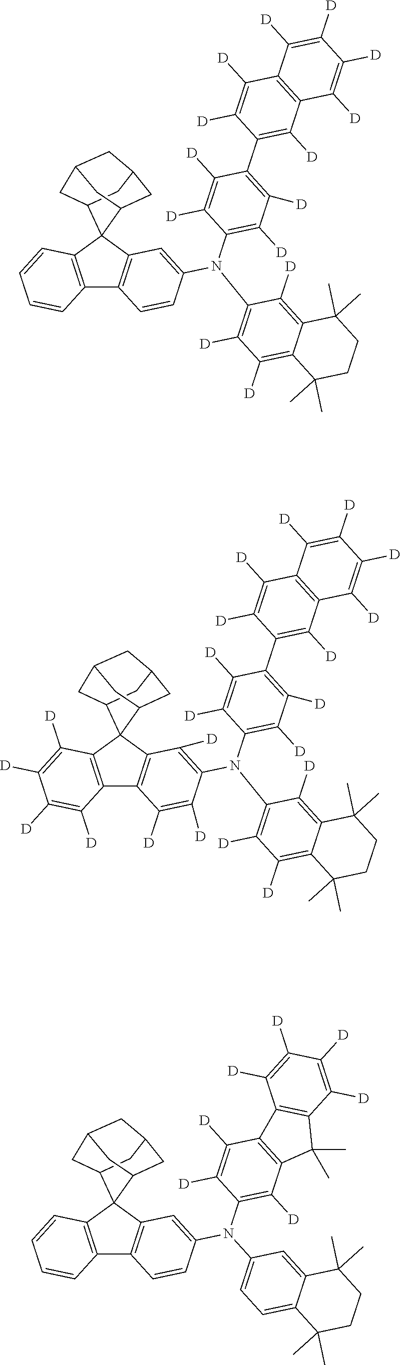

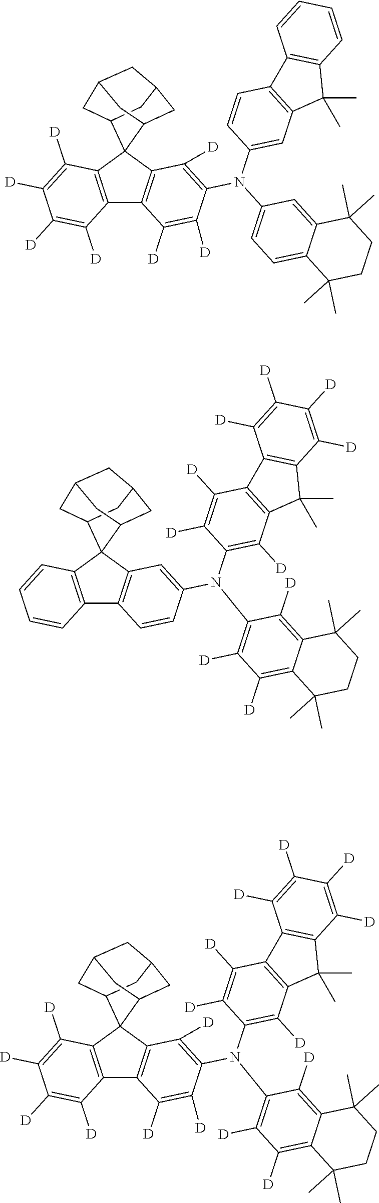 [SMILES: [2H]c1c([2H])c([2H])c2c(c1[2H])-c1c([2H])c([2H])c(N(c3ccc4c(c3)C(C)(C)CCC4(C)C)c3ccc4c(c3)C(C)(C)c3ccccc3-4)c([2H])c1C21C2CC3CC(C2)CC1C3 ; [2H]c1c([2H])c([2H])c2c(c1[2H])-c1c([2H])c([2H])c(N(c3ccc4c(c3)C3(c5ccccc5-4)C4CC5CC(C4)CC3C5)c3c([2H])c([2H])c4c(c3[2H])C(C)(C)CCC4(C)C)c([2H])c1C2(C)C ; [2H]c1c([2H])c([2H])c2c(c1[2H])-c1c([2H])c([2H])c(N(c3c([2H])c([2H])c4c(c3[2H])C(C)(C)CCC4(C)C)c3c([2H])c([2H])c4c(c3[2H])C3(c5c([2H])c([2H])c([2H])c([2H])c5-4)C4CC5CC(C4)CC3C5)c([2H])c1C2(C)C]

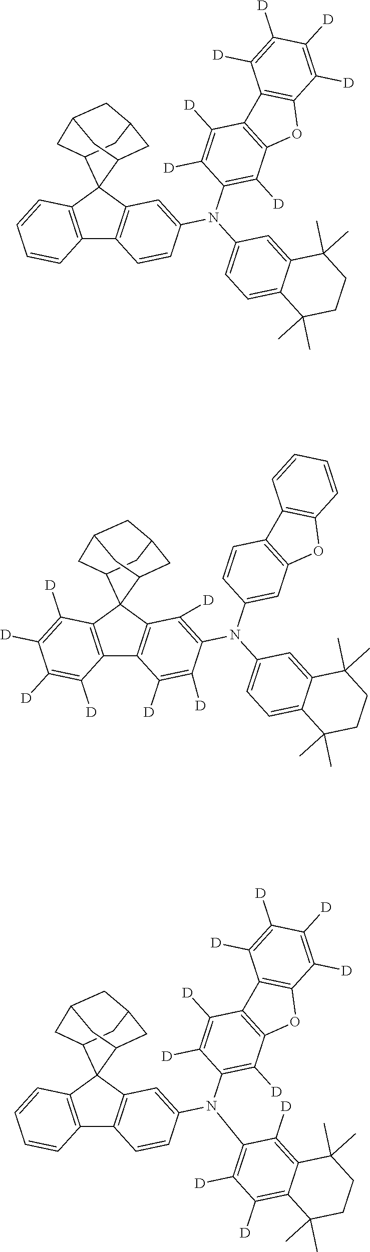 [SMILES: [2H]c1c([2H])c([2H])c2c(oc3c([2H])c(N(c4ccc5c(c4)C(C)(C)CCC5(C)C)c4ccc5c(c4)C4(c6ccccc6-5)C5CC6CC(C5)CC4C6)c([2H])c([2H])c32)c1[2H] ; [2H]c1c([2H])c([2H])c2c(c1[2H])-c1c([2H])c([2H])c(N(c3ccc4c(c3)C(C)(C)CCC4(C)C)c3ccc4c(c3)oc3ccccc34)c([2H])c1C21C2CC3CC(C2)CC1C3 ; [2H]c1c([2H])c2c(c([2H])c1N(c1ccc3c(c1)C1(c4ccccc4-3)C3CC4CC(C3)CC1C4)c1c([2H])c([2H])c3c(oc4c([2H])c([2H])c([2H])c([2H])c43)c1[2H])C(C)(C)CCC2(C)C]

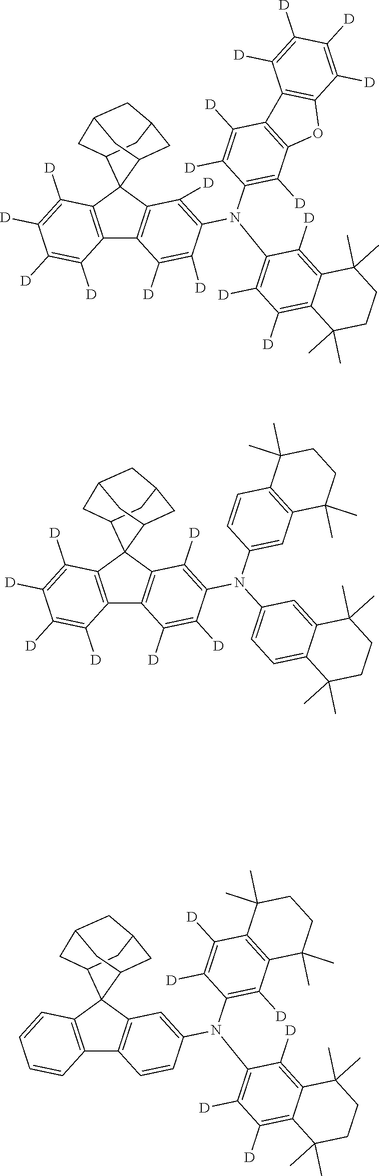 [SMILES: [2H]c1c([2H])c([2H])c2c(c1[2H])-c1c([2H])c([2H])c(N(c3c([2H])c([2H])c4c(c3[2H])C(C)(C)CCC4(C)C)c3c([2H])c([2H])c4c(oc5c([2H])c([2H])c([2H])c([2H])c54)c3[2H])c([2H])c1C21C2CC3CC(C2)CC1C3 ; [2H]c1c([2H])c([2H])c2c(c1[2H])-c1c([2H])c([2H])c(N(c3ccc4c(c3)C(C)(C)CCC4(C)C)c3ccc4c(c3)C(C)(C)CCC4(C)C)c([2H])c1C21C2CC3CC(C2)CC1C3 ; [2H]c1c([2H])c2c(c([2H])c1N(c1ccc3c(c1)C1(c4ccccc4-3)C3CC4CC(C3)CC1C4)c1c([2H])c([2H])c3c(c1[2H])C(C)(C)CCC3(C)C)C(C)(C)CCC2(C)C]

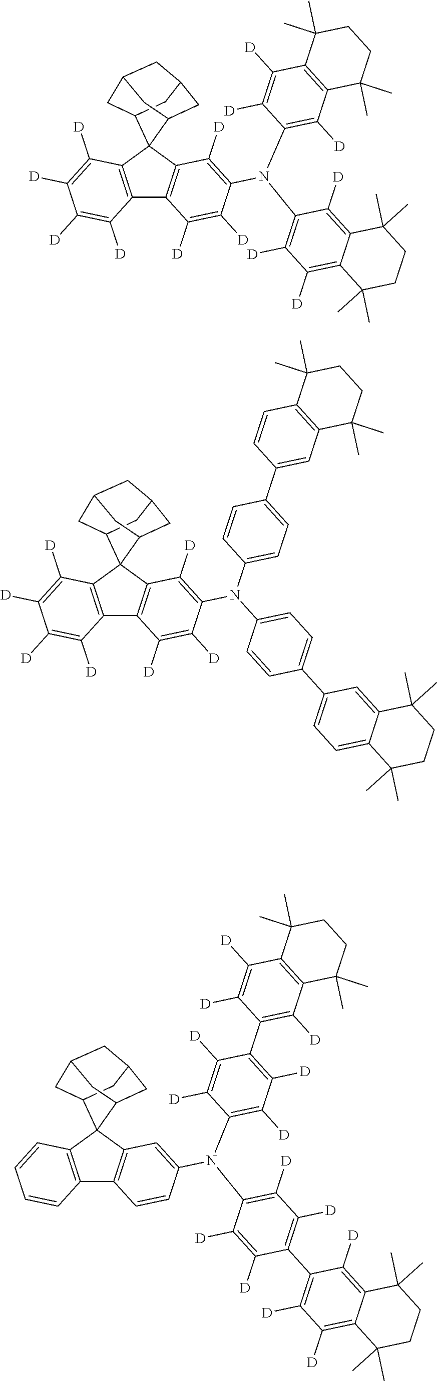

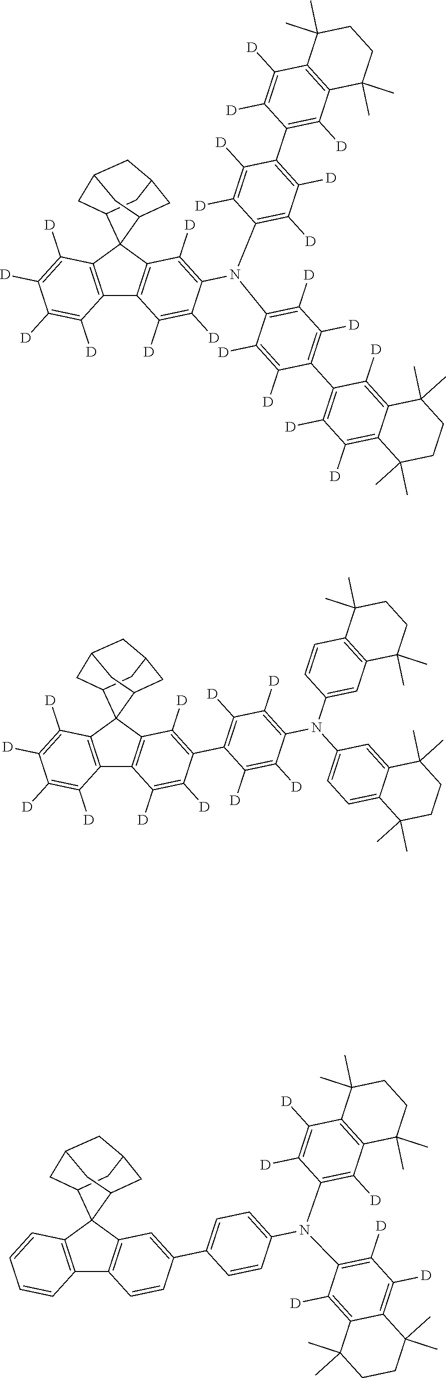

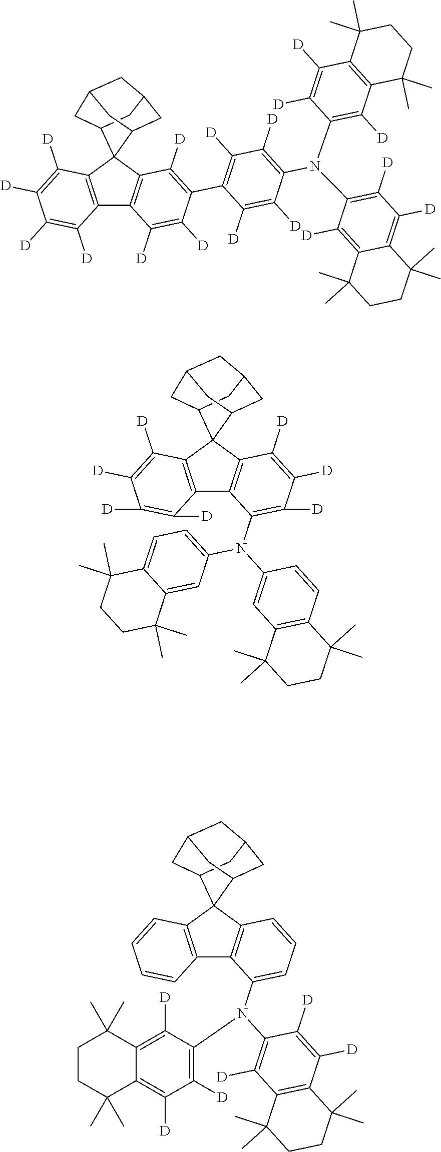

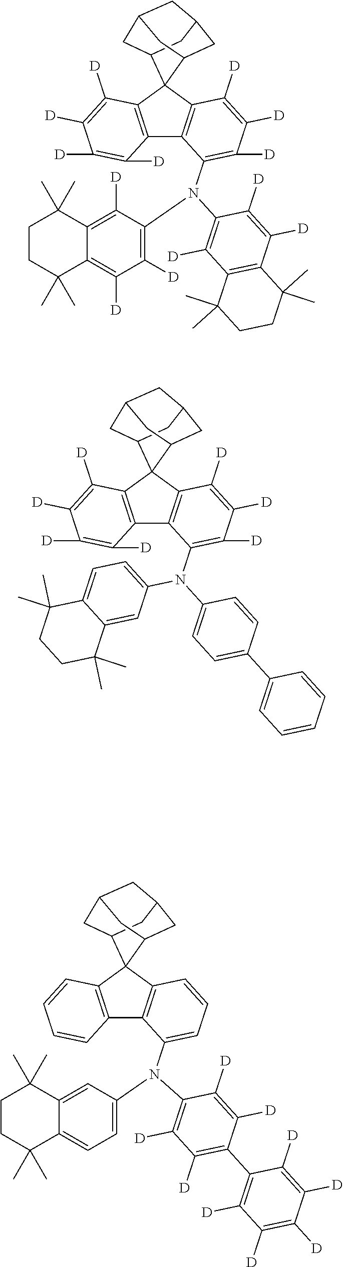

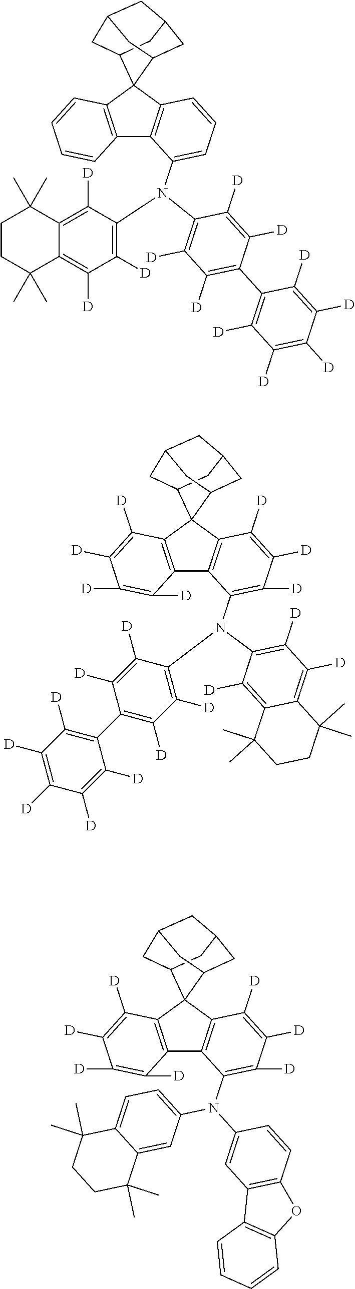

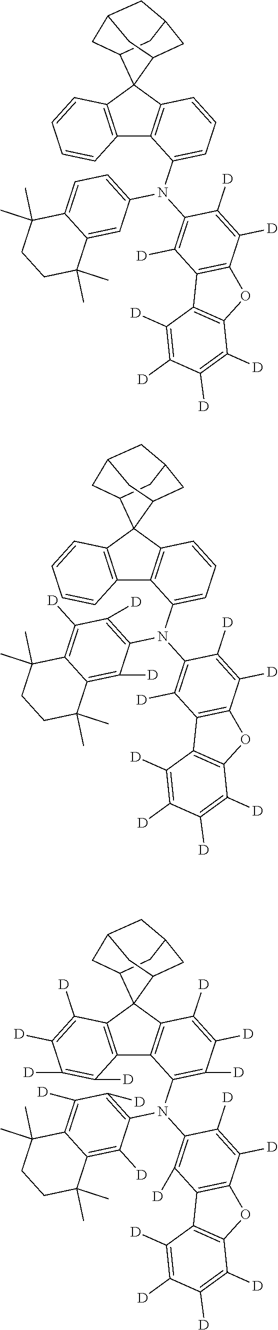 [SMILES: [2H]c1c([2H])c([2H])c2c(oc3c([2H])c([2H])c(N(c4ccc5c(c4)C(C)(C)CCC5(C)C)c4cccc5c4-c4ccccc4C54C5CC6CC(C5)CC4C6)c([2H])c32)c1[2H] ; [2H]c1c([2H])c2c(c([2H])c1N(c1cccc3c1-c1ccccc1C31C3CC4CC(C3)CC1C4)c1c([2H])c([2H])c3oc4c([2H])c([2H])c([2H])c([2H])c4c3c1[2H])C(C)(C)CCC2(C)C ; [2H]c1c([2H])c([2H])c2c(c1[2H])-c1c(N(c3c([2H])c([2H])c4c(c3[2H])C(C)(C)CCC4(C)C)c3c([2H])c([2H])c4oc5c([2H])c([2H])c([2H])c([2H])c5c4c3[2H])c([2H])c([2H])c([2H])c1C21C2CC3CC(C2)CC1C3]

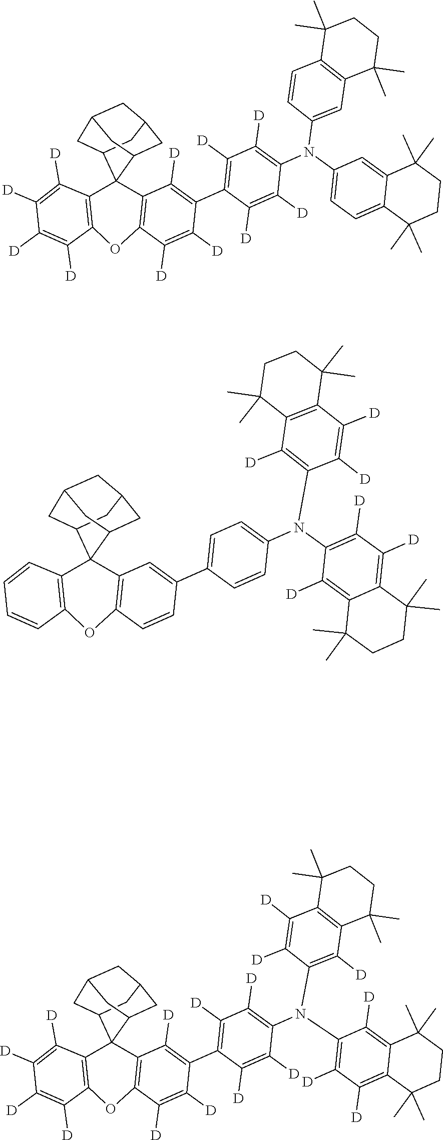

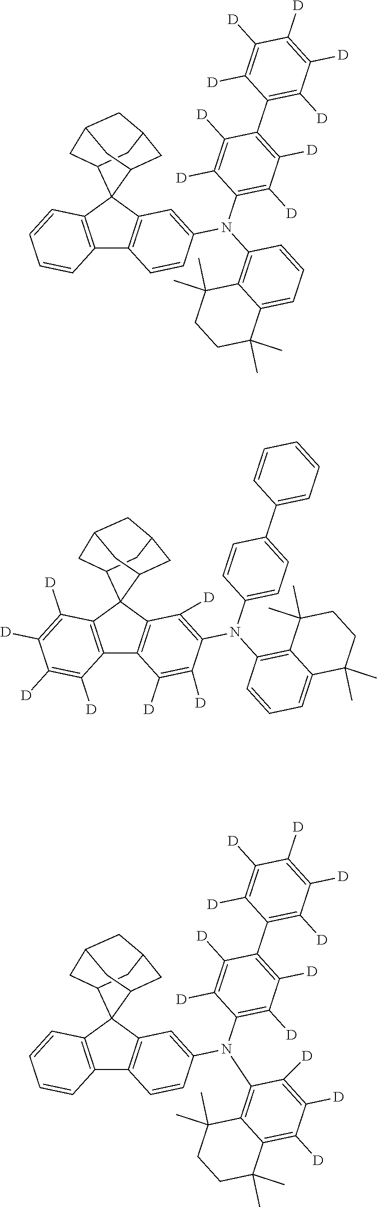 [SMILES: [2H]c1c([2H])c([2H])c(-c2c([2H])c([2H])c(N(c3ccc4c(c3)C3(c5ccccc5-4)C4CC5CC(C4)CC3C5)c3cccc4c3C(C)(C)CCC4(C)C)c([2H])c2[2H])c([2H])c1[2H] ; [2H]c1c([2H])c([2H])c2c(c1[2H])-c1c([2H])c([2H])c(N(c3ccc(-c4ccccc4)cc3)c3cccc4c3C(C)(C)CCC4(C)C)c([2H])c1C21C2CC3CC(C2)CC1C3 ; [2H]c1c([2H])c([2H])c(-c2c([2H])c([2H])c(N(c3ccc4c(c3)C3(c5ccccc5-4)C4CC5CC(C4)CC3C5)c3c([2H])c([2H])c([2H])c4c3C(C)(C)CCC4(C)C)c([2H])c2[2H])c([2H])c1[2H]]

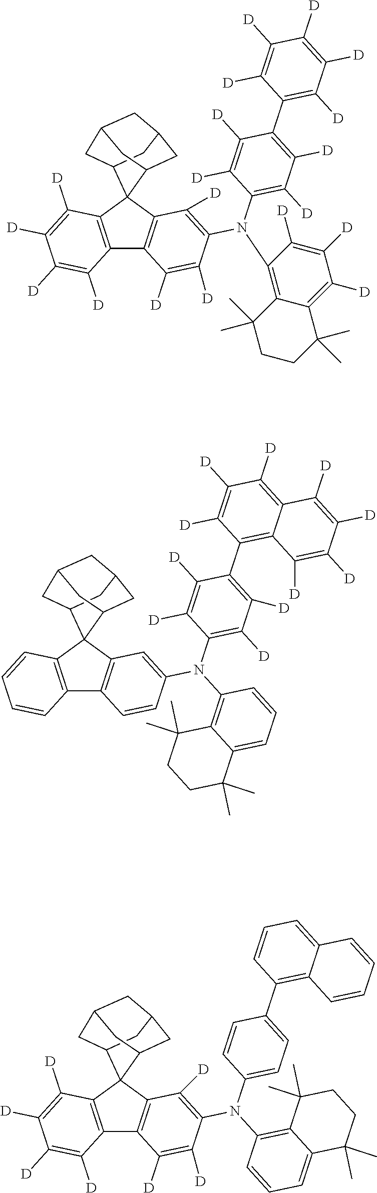 [SMILES: [2H]c1c([2H])c([2H])c(-c2c([2H])c([2H])c(N(c3c([2H])c([2H])c4c(c3[2H])C3(c5c([2H])c([2H])c([2H])c([2H])c5-4)C4CC5CC(C4)CC3C5)c3c([2H])c([2H])c([2H])c4c3C(C)(C)CCC4(C)C)c([2H])c2[2H])c([2H])c1[2H] ; [2H]c1c([2H])c(N(c2ccc3c(c2)C2(c4ccccc4-3)C3CC4CC(C3)CC2C4)c2cccc3c2C(C)(C)CCC3(C)C)c([2H])c([2H])c1-c1c([2H])c([2H])c([2H])c2c([2H])c([2H])c([2H])c([2H])c12 ; [2H]c1c([2H])c([2H])c2c(c1[2H])-c1c([2H])c([2H])c(N(c3ccc(-c4cccc5ccccc45)cc3)c3cccc4c3C(C)(C)CCC4(C)C)c([2H])c1C21C2CC3CC(C2)CC1C3]

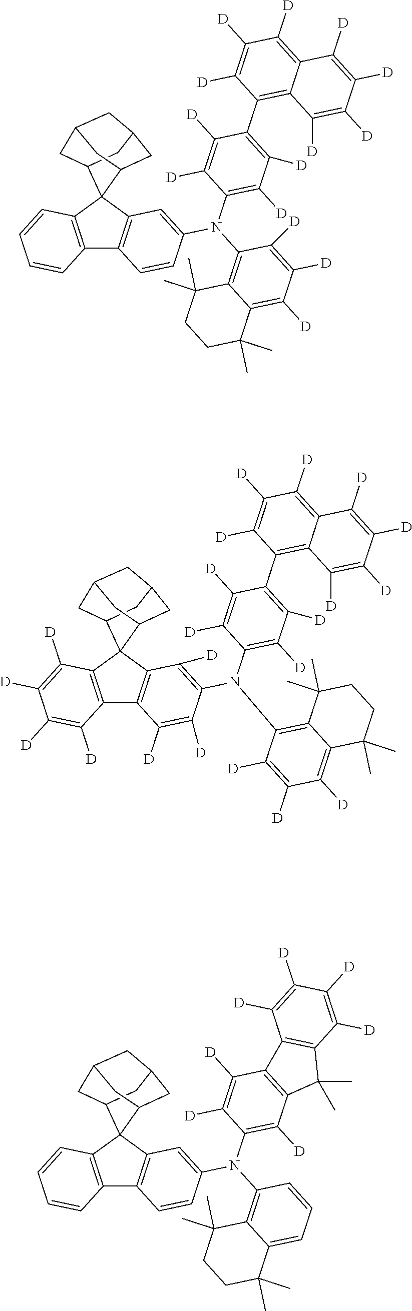

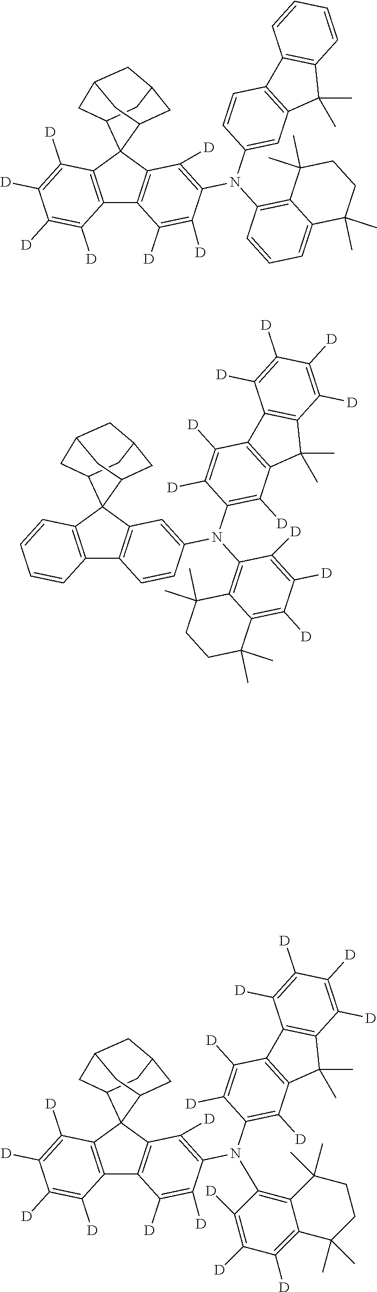 [SMILES: [2H]c1c([2H])c([2H])c2c(c1[2H])-c1c([2H])c([2H])c(N(c3ccc4c(c3)C(C)(C)c3ccccc3-4)c3cccc4c3C(C)(C)CCC4(C)C)c([2H])c1C21C2CC3CC(C2)CC1C3 ; [2H]c1c([2H])c([2H])c2c(c1[2H])-c1c([2H])c([2H])c(N(c3ccc4c(c3)C3(c5ccccc5-4)C4CC5CC(C4)CC3C5)c3c([2H])c([2H])c([2H])c4c3C(C)(C)CCC4(C)C)c([2H])c1C2(C)C ; [2H]c1c([2H])c([2H])c2c(c1[2H])-c1c([2H])c([2H])c(N(c3c([2H])c([2H])c4c(c3[2H])C3(c5c([2H])c([2H])c([2H])c([2H])c5-4)C4CC5CC(C4)CC3C5)c3c([2H])c([2H])c([2H])c4c3C(C)(C)CCC4(C)C)c([2H])c1C2(C)C]

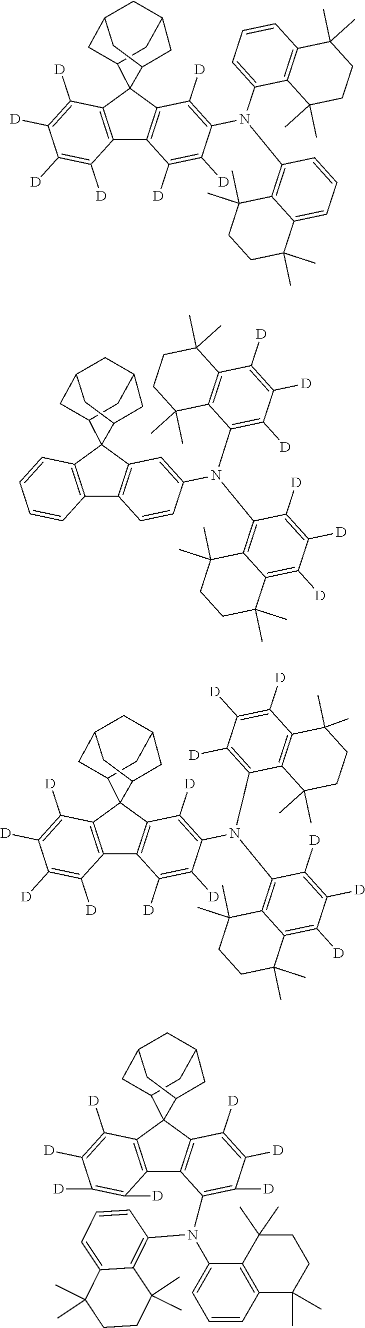

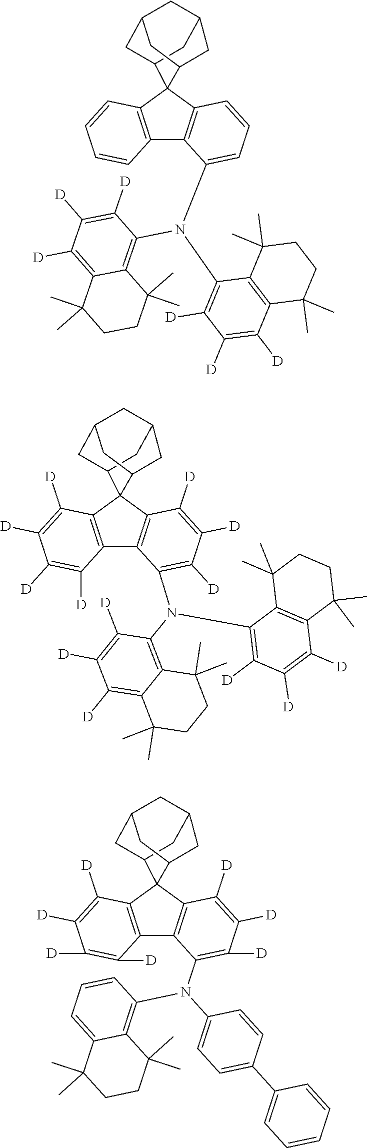

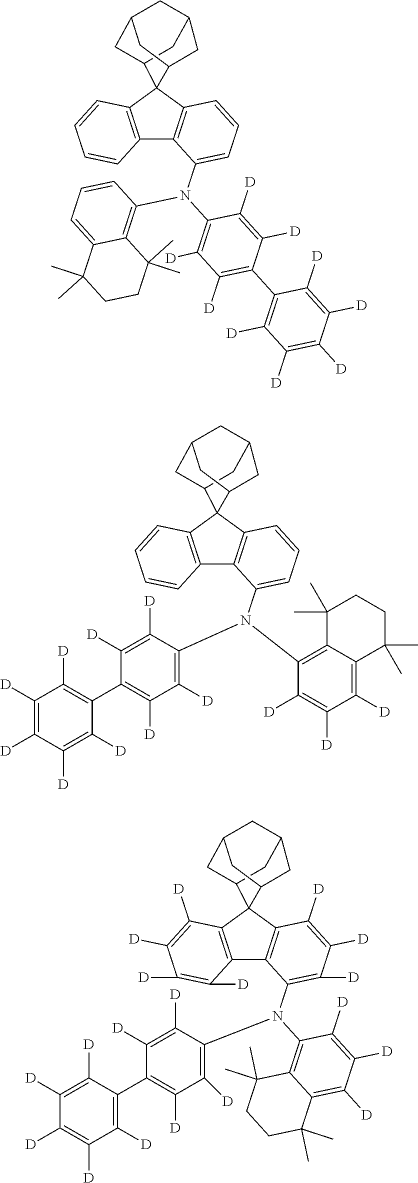 [SMILES: [2H]c1c([2H])c([2H])c(-c2c([2H])c([2H])c(N(c3cccc4c3-c3ccccc3C43C4CC5CC(C4)CC3C5)c3cccc4c3C(C)(C)CCC4(C)C)c([2H])c2[2H])c([2H])c1[2H] ; [2H]c1c([2H])c([2H])c(-c2c([2H])c([2H])c(N(c3cccc4c3-c3ccccc3C43C4CC5CC(C4)CC3C5)c3c([2H])c([2H])c([2H])c4c3C(C)(C)CCC4(C)C)c([2H])c2[2H])c([2H])c1[2H] ; [2H]c1c([2H])c([2H])c(-c2c([2H])c([2H])c(N(c3c([2H])c([2H])c([2H])c4c3-c3c([2H])c([2H])c([2H])c([2H])c3C43C4CC5CC(C4)CC3C5)c3c([2H])c([2H])c([2H])c4c3C(C)(C)CCC4(C)C)c([2H])c2[2H])c([2H])c1[2H]]

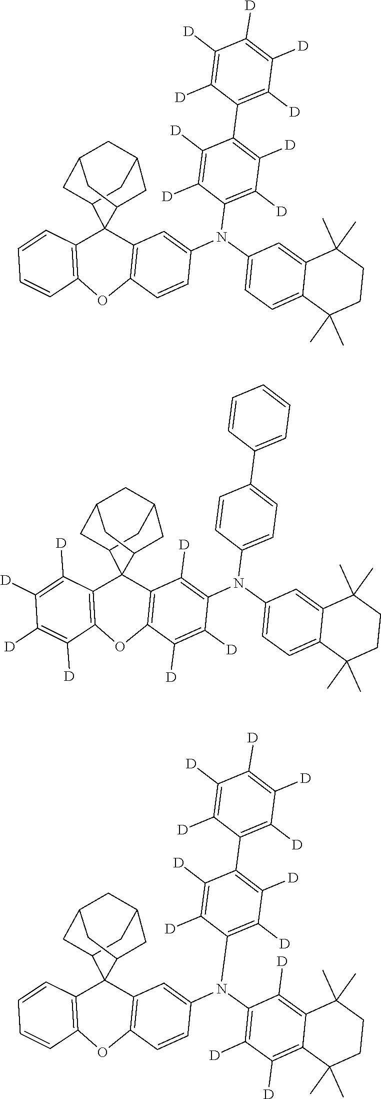 [SMILES: [2H]c1c([2H])c([2H])c(-c2c([2H])c([2H])c(N(c3ccc4c(c3)C(C)(C)CCC4(C)C)c3ccc4c(c3)C3(c5ccccc5O4)C4CC5CC(C4)CC3C5)c([2H])c2[2H])c([2H])c1[2H] ; [2H]c1c([2H])c([2H])c2c(c1[2H])Oc1c([2H])c([2H])c(N(c3ccc(-c4ccccc4)cc3)c3ccc4c(c3)C(C)(C)CCC4(C)C)c([2H])c1C21C2CC3CC(C2)CC1C3 ; [2H]c1c([2H])c([2H])c(-c2c([2H])c([2H])c(N(c3ccc4c(c3)C3(c5ccccc5O4)C4CC5CC(C4)CC3C5)c3c([2H])c([2H])c4c(c3[2H])C(C)(C)CCC4(C)C)c([2H])c2[2H])c([2H])c1[2H]]

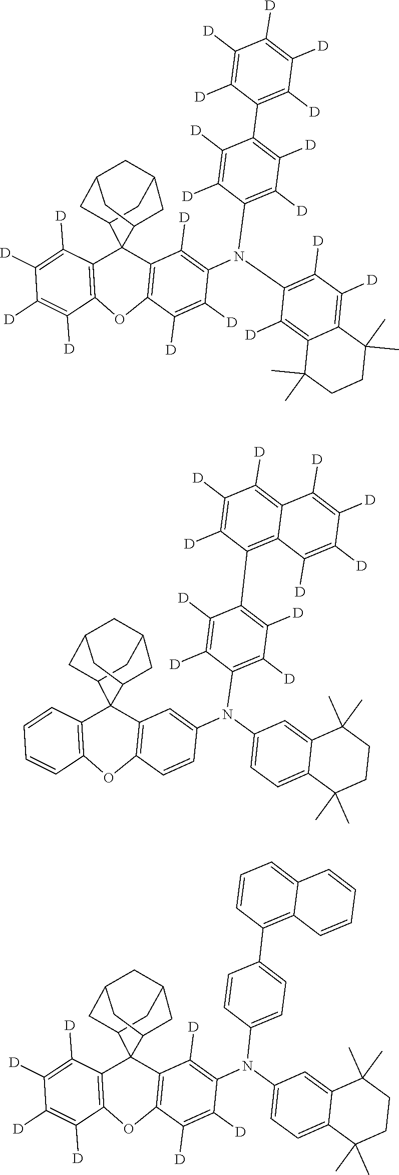 [SMILES: [2H]c1c([2H])c([2H])c(-c2c([2H])c([2H])c(N(c3c([2H])c([2H])c4c(c3[2H])C(C)(C)CCC4(C)C)c3c([2H])c([2H])c4c(c3[2H])C3(c5c([2H])c([2H])c([2H])c([2H])c5O4)C4CC5CC(C4)CC3C5)c([2H])c2[2H])c([2H])c1[2H] ; [2H]c1c([2H])c(N(c2ccc3c(c2)C(C)(C)CCC3(C)C)c2ccc3c(c2)C2(c4ccccc4O3)C3CC4CC(C3)CC2C4)c([2H])c([2H])c1-c1c([2H])c([2H])c([2H])c2c([2H])c([2H])c([2H])c([2H])c12 ; [2H]c1c([2H])c([2H])c2c(c1[2H])Oc1c([2H])c([2H])c(N(c3ccc(-c4cccc5ccccc45)cc3)c3ccc4c(c3)C(C)(C)CCC4(C)C)c([2H])c1C21C2CC3CC(C2)CC1C3]

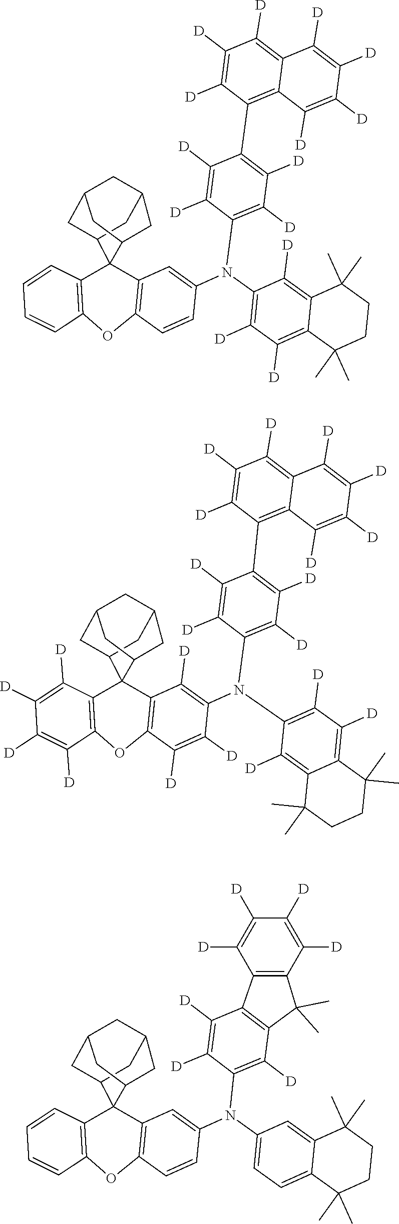

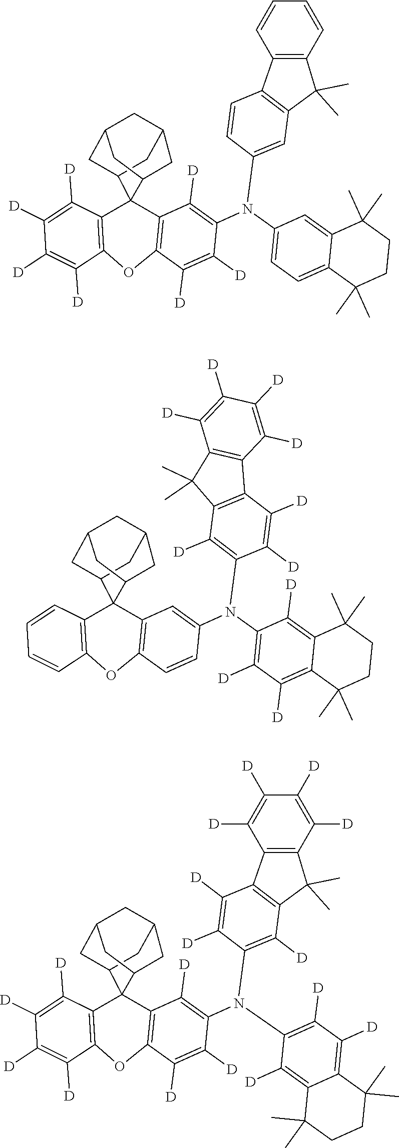 [SMILES: [2H]c1c([2H])c([2H])c2c(c1[2H])Oc1c([2H])c([2H])c(N(c3ccc4c(c3)C(C)(C)CCC4(C)C)c3ccc4c(c3)C(C)(C)c3ccccc3-4)c([2H])c1C21C2CC3CC(C2)CC1C3 ; [2H]c1c([2H])c([2H])c2c(c1[2H])-c1c([2H])c([2H])c(N(c3ccc4c(c3)C3(c5ccccc5O4)C4CC5CC(C4)CC3C5)c3c([2H])c([2H])c4c(c3[2H])C(C)(C)CCC4(C)C)c([2H])c1C2(C)C ; [2H]c1c([2H])c([2H])c2c(c1[2H])Oc1c([2H])c([2H])c(N(c3c([2H])c([2H])c4c(c3[2H])C(C)(C)CCC4(C)C)c3c([2H])c([2H])c4c(c3[2H])C(C)(C)c3c([2H])c([2H])c([2H])c([2H])c3-4)c([2H])c1C21C2CC3CC(C2)CC1C3]

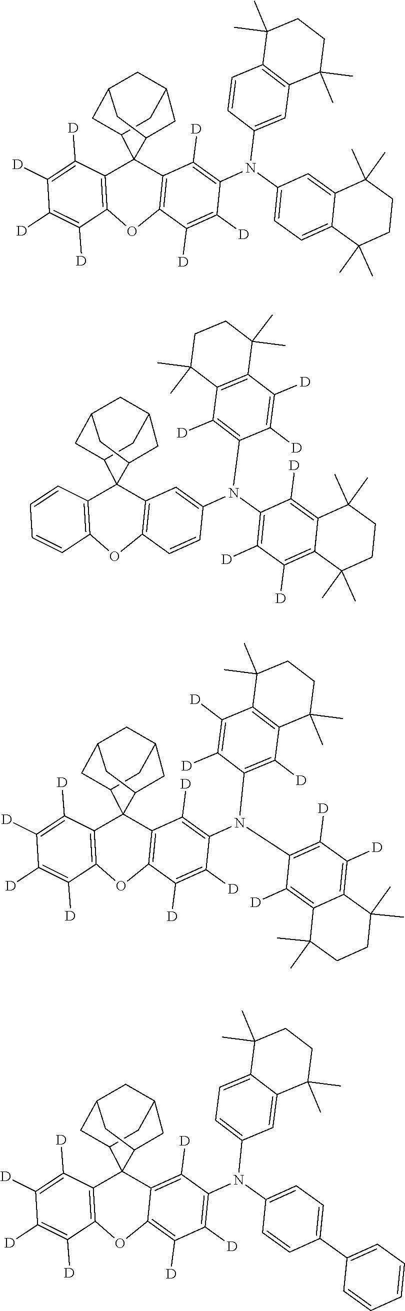

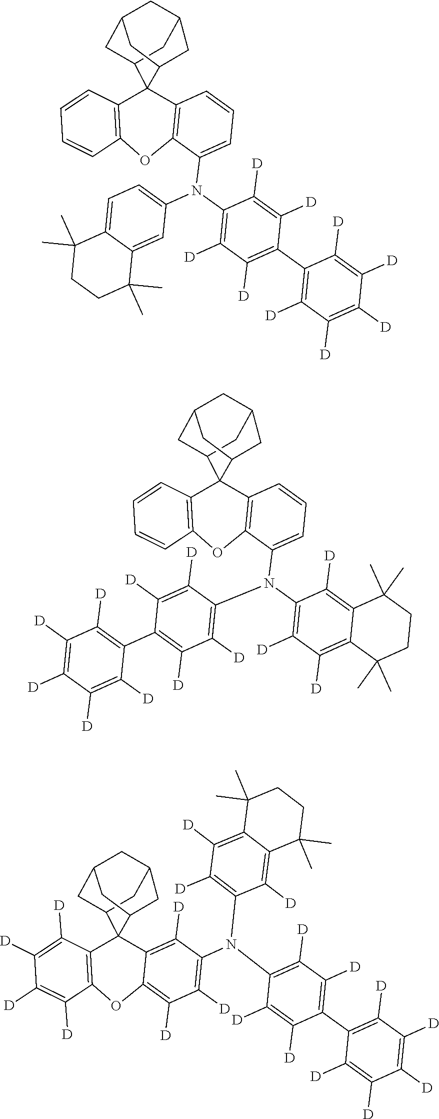 [SMILES: [2H]c1c([2H])c([2H])c(-c2c([2H])c([2H])c(N(c3ccc4c(c3)C(C)(C)CCC4(C)C)c3cccc4c3Oc3ccccc3C43C4CC5CC(C4)CC3C5)c([2H])c2[2H])c([2H])c1[2H] ; [2H]c1c([2H])c([2H])c(-c2c([2H])c([2H])c(N(c3cccc4c3Oc3ccccc3C43C4CC5CC(C4)CC3C5)c3c([2H])c([2H])c4c(c3[2H])C(C)(C)CCC4(C)C)c([2H])c2[2H])c([2H])c1[2H] ; [2H]c1c([2H])c([2H])c(-c2c([2H])c([2H])c(N(c3c([2H])c([2H])c4c(c3[2H])C(C)(C)CCC4(C)C)c3c([2H])c([2H])c4c(c3[2H])C3(c5c([2H])c([2H])c([2H])c([2H])c5O4)C4CC5CC(C4)CC3C5)c([2H])c2[2H])c([2H])c1[2H]]

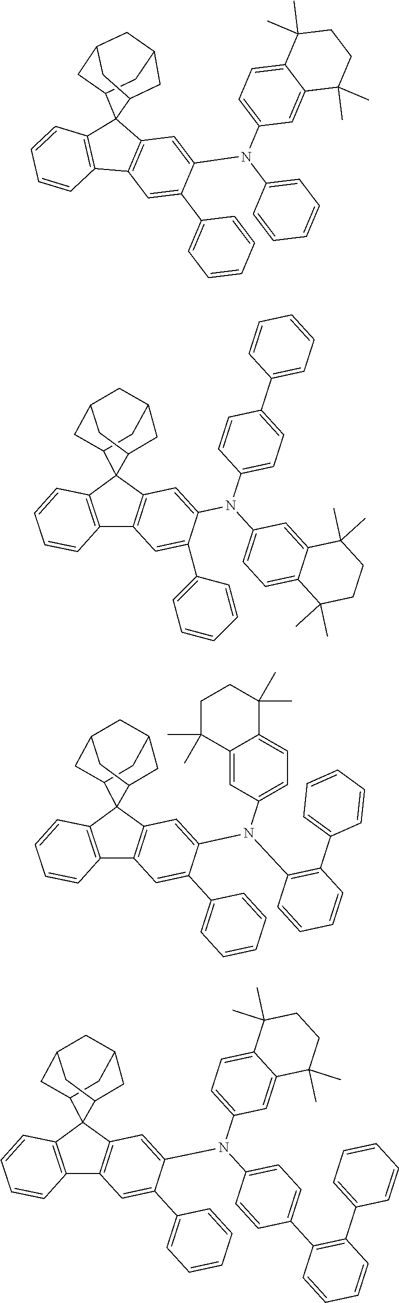

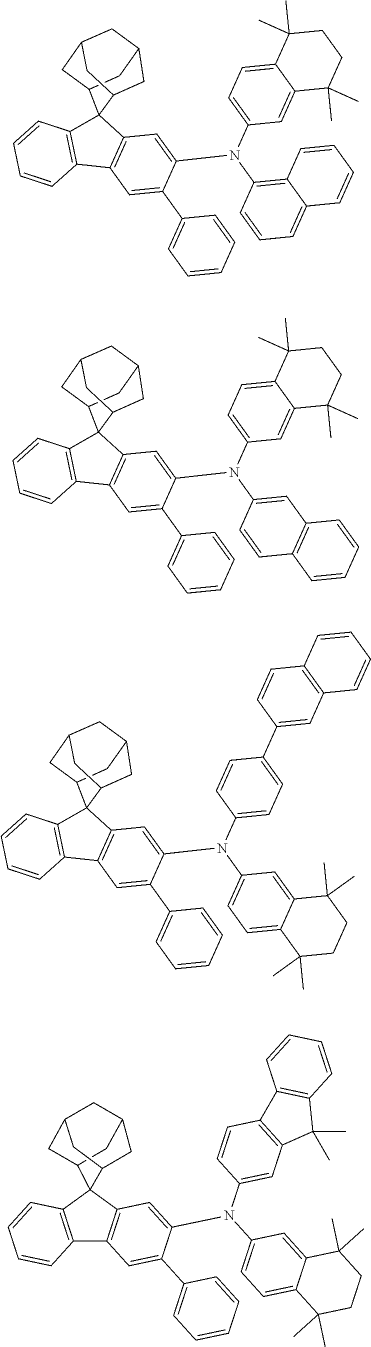 [SMILES: CC1(C)CCC(C)(C)c2cc(N(c3cc4c(cc3-c3ccccc3)-c3ccccc3C43C4CC5CC(C4)CC3C5)c3cccc4ccccc34)ccc21 ; CC1(C)CCC(C)(C)c2cc(N(c3ccc4ccccc4c3)c3cc4c(cc3-c3ccccc3)-c3ccccc3C43C4CC5CC(C4)CC3C5)ccc21 ; CC1(C)CCC(C)(C)c2cc(N(c3ccc(-c4ccc5ccccc5c4)cc3)c3cc4c(cc3-c3ccccc3)-c3ccccc3C43C4CC5CC(C4)CC3C5)ccc21 ; CC1(C)CCC(C)(C)c2cc(N(c3ccc4c(c3)C(C)(C)c3ccccc3-4)c3cc4c(cc3-c3ccccc3)-c3ccccc3C43C4CC5CC(C4)CC3C5)ccc21]

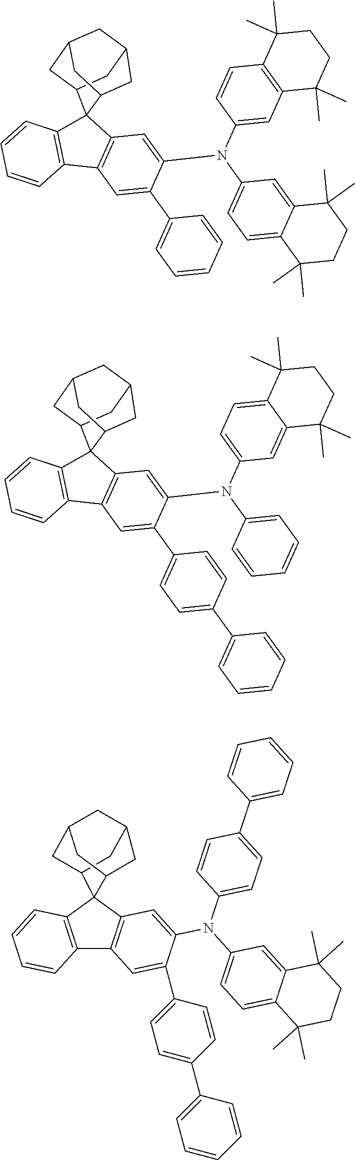 [SMILES: CC1(C)CCC(C)(C)c2cc(N(c3ccc4c(c3)C(C)(C)CCC4(C)C)c3cc4c(cc3-c3ccccc3)-c3ccccc3C43C4CC5CC(C4)CC3C5)ccc21 ; CC1(C)CCC(C)(C)c2cc(N(c3ccccc3)c3cc4c(cc3-c3ccc(-c5ccccc5)cc3)-c3ccccc3C43C4CC5CC(C4)CC3C5)ccc21 ; CC1(C)CCC(C)(C)c2cc(N(c3ccc(-c4ccccc4)cc3)c3cc4c(cc3-c3ccc(-c5ccccc5)cc3)-c3ccccc3C43C4CC5CC(C4)CC3C5)ccc21]

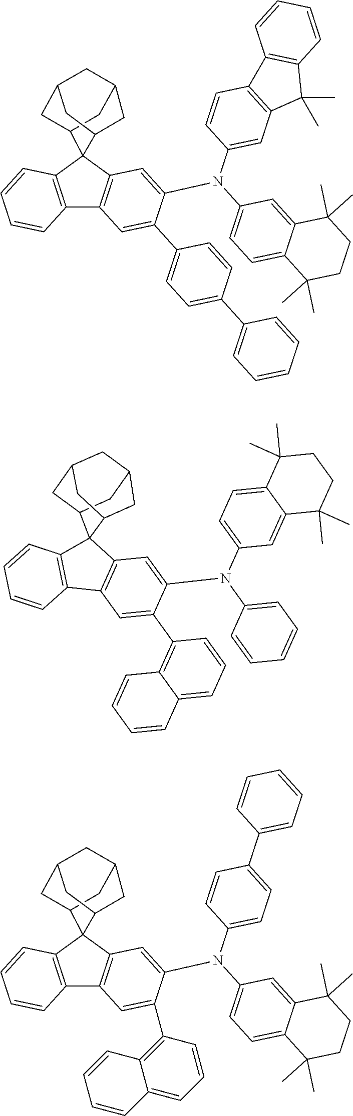 [SMILES: CC1(C)CCC(C)(C)c2cc(N(c3ccc4c(c3)C(C)(C)c3ccccc3-4)c3cc4c(cc3-c3ccc(-c5ccccc5)cc3)-c3ccccc3C43C4CC5CC(C4)CC3C5)ccc21 ; CC1(C)CCC(C)(C)c2cc(N(c3ccccc3)c3cc4c(cc3-c3cccc5ccccc35)-c3ccccc3C43C4CC5CC(C4)CC3C5)ccc21 ; CC1(C)CCC(C)(C)c2cc(N(c3ccc(-c4ccccc4)cc3)c3cc4c(cc3-c3cccc5ccccc35)-c3ccccc3C43C4CC5CC(C4)CC3C5)ccc21]

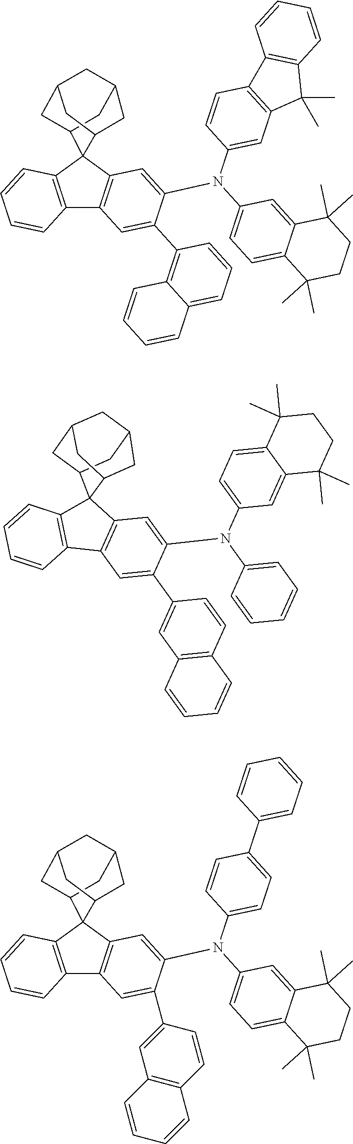 [SMILES: CC1(C)CCC(C)(C)c2cc(N(c3ccc4c(c3)C(C)(C)c3ccccc3-4)c3cc4c(cc3-c3cccc5ccccc35)-c3ccccc3C43C4CC5CC(C4)CC3C5)ccc21 ; CC1(C)CCC(C)(C)c2cc(N(c3ccccc3)c3cc4c(cc3-c3ccc5ccccc5c3)-c3ccccc3C43C4CC5CC(C4)CC3C5)ccc21 ; CC1(C)CCC(C)(C)c2cc(N(c3ccc(-c4ccccc4)cc3)c3cc4c(cc3-c3ccc5ccccc5c3)-c3ccccc3C43C4CC5CC(C4)CC3C5)ccc21]

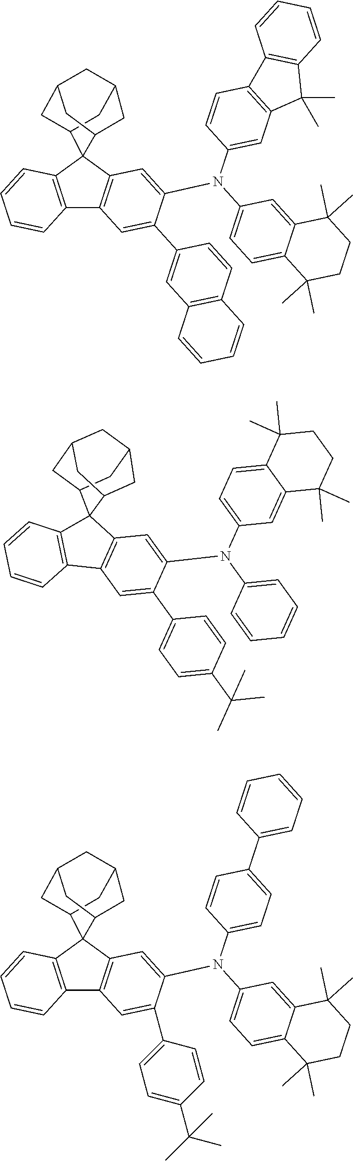 [SMILES: CC1(C)CCC(C)(C)c2cc(N(c3ccc4c(c3)C(C)(C)c3ccccc3-4)c3cc4c(cc3-c3ccc5ccccc5c3)-c3ccccc3C43C4CC5CC(C4)CC3C5)ccc21 ; CC(C)(C)c1ccc(-c2cc3c(cc2N(c2ccccc2)c2ccc4c(c2)C(C)(C)CCC4(C)C)C2(c4ccccc4-3)C3CC4CC(C3)CC2C4)cc1 ; CC(C)(C)c1ccc(-c2cc3c(cc2N(c2ccc(-c4ccccc4)cc2)c2ccc4c(c2)C(C)(C)CCC4(C)C)C2(c4ccccc4-3)C3CC4CC(C3)CC2C4)cc1]

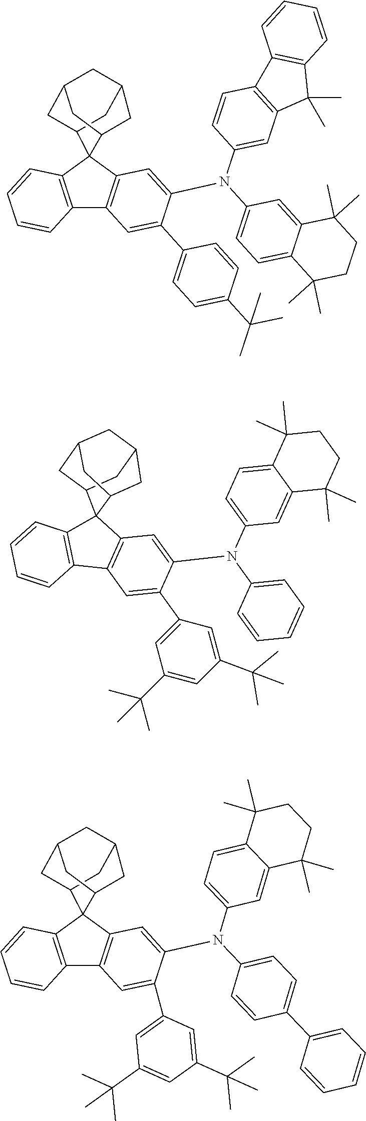 [SMILES: CC(C)(C)c1ccc(-c2cc3c(cc2N(c2ccc4c(c2)C(C)(C)CCC4(C)C)c2ccc4c(c2)C(C)(C)c2ccccc2-4)C2(c4ccccc4-3)C3CC4CC(C3)CC2C4)cc1 ; CC(C)(C)c1cc(-c2cc3c(cc2N(c2ccccc2)c2ccc4c(c2)C(C)(C)CCC4(C)C)C2(c4ccccc4-3)C3CC4CC(C3)CC2C4)cc(C(C)(C)C)c1 ; CC(C)(C)c1cc(-c2cc3c(cc2N(c2ccc(-c4ccccc4)cc2)c2ccc4c(c2)C(C)(C)CCC4(C)C)C2(c4ccccc4-3)C3CC4CC(C3)CC2C4)cc(C(C)(C)C)c1]

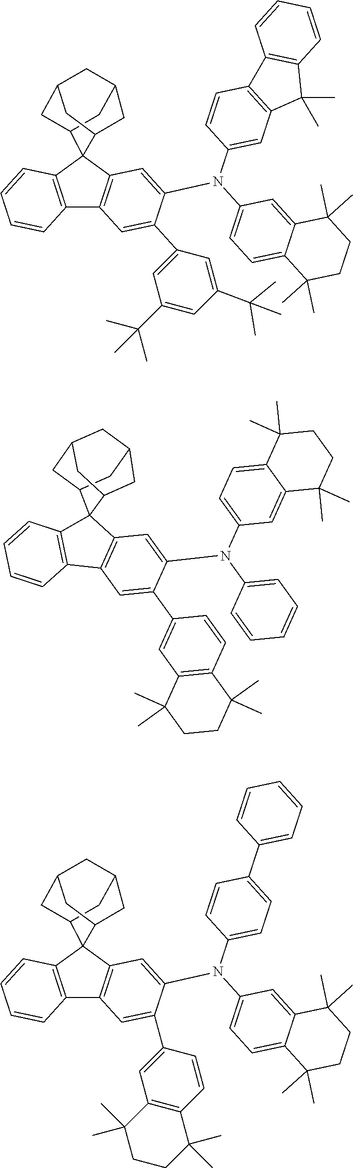 [SMILES: CC(C)(C)c1cc(-c2cc3c(cc2N(c2ccc4c(c2)C(C)(C)CCC4(C)C)c2ccc4c(c2)C(C)(C)c2ccccc2-4)C2(c4ccccc4-3)C3CC4CC(C3)CC2C4)cc(C(C)(C)C)c1 ; CC1(C)CCC(C)(C)c2cc(-c3cc4c(cc3N(c3ccccc3)c3ccc5c(c3)C(C)(C)CCC5(C)C)C3(c5ccccc5-4)C4CC5CC(C4)CC3C5)ccc21 ; CC1(C)CCC(C)(C)c2cc(-c3cc4c(cc3N(c3ccc(-c5ccccc5)cc3)c3ccc5c(c3)C(C)(C)CCC5(C)C)C3(c5ccccc5-4)C4CC5CC(C4)CC3C5)ccc21]

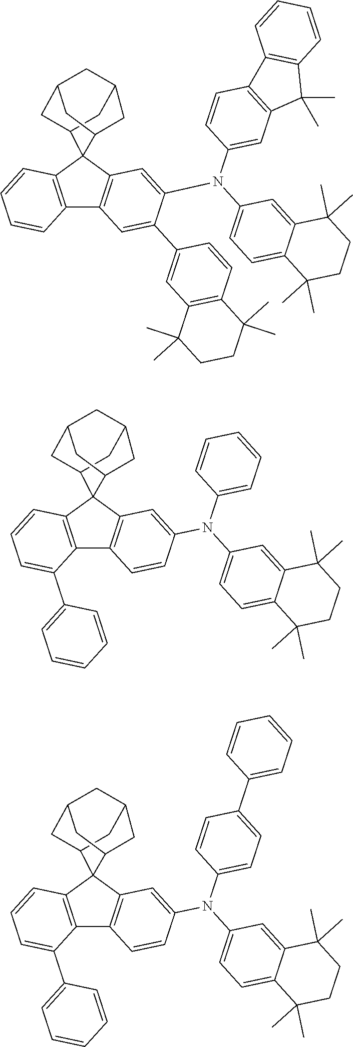 [SMILES: CC1(C)CCC(C)(C)c2cc(-c3cc4c(cc3N(c3ccc5c(c3)C(C)(C)CCC5(C)C)c3ccc5c(c3)C(C)(C)c3ccccc3-5)C3(c5ccccc5-4)C4CC5CC(C4)CC3C5)ccc21 ; CC1(C)CCC(C)(C)c2cc(N(c3ccccc3)c3ccc4c(c3)C3(c5cccc(-c6ccccc6)c5-4)C4CC5CC(C4)CC3C5)ccc21 ; CC1(C)CCC(C)(C)c2cc(N(c3ccc(-c4ccccc4)cc3)c3ccc4c(c3)C3(c5cccc(-c6ccccc6)c5-4)C4CC5CC(C4)CC3C5)ccc21]

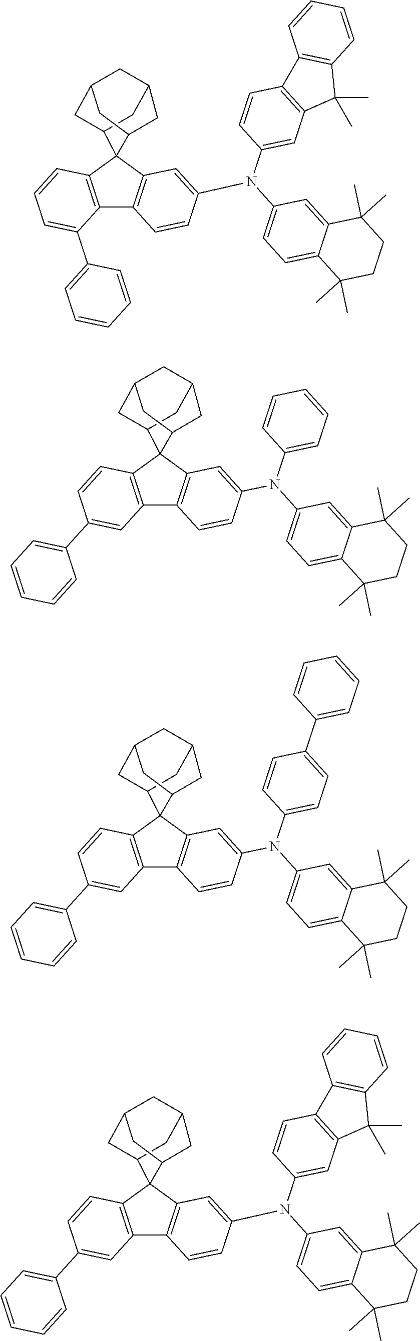 [SMILES: CC1(C)CCC(C)(C)c2cc(N(c3ccc4c(c3)C(C)(C)c3ccccc3-4)c3ccc4c(c3)C3(c5cccc(-c6ccccc6)c5-4)C4CC5CC(C4)CC3C5)ccc21 ; CC1(C)CCC(C)(C)c2cc(N(c3ccccc3)c3ccc4c(c3)C3(c5ccc(-c6ccccc6)cc5-4)C4CC5CC(C4)CC3C5)ccc21 ; CC1(C)CCC(C)(C)c2cc(N(c3ccc(-c4ccccc4)cc3)c3ccc4c(c3)C3(c5ccc(-c6ccccc6)cc5-4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5ccc(-c6ccccc6)cc5-4)C4CC5CC(C4)CC3C5)ccc21]

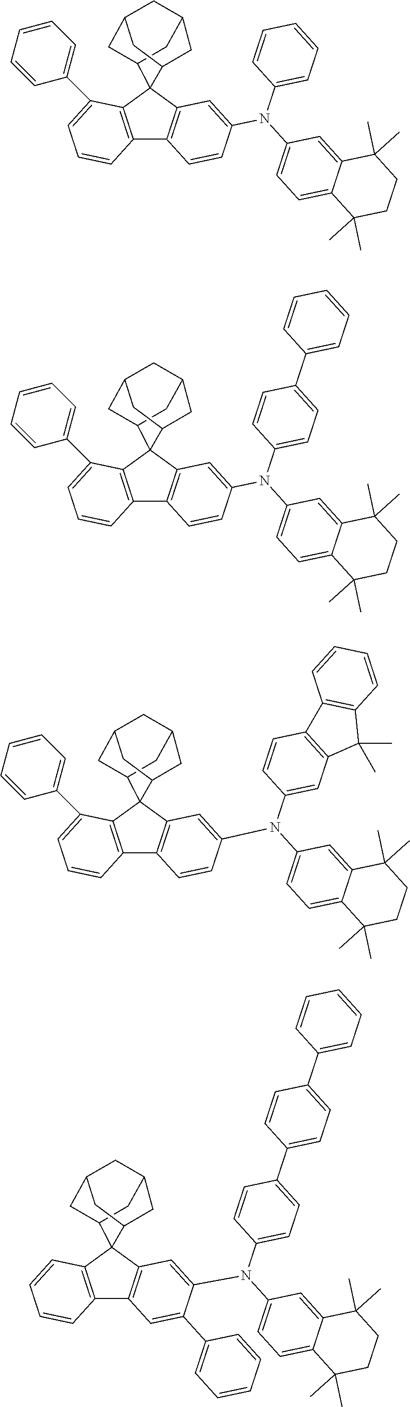

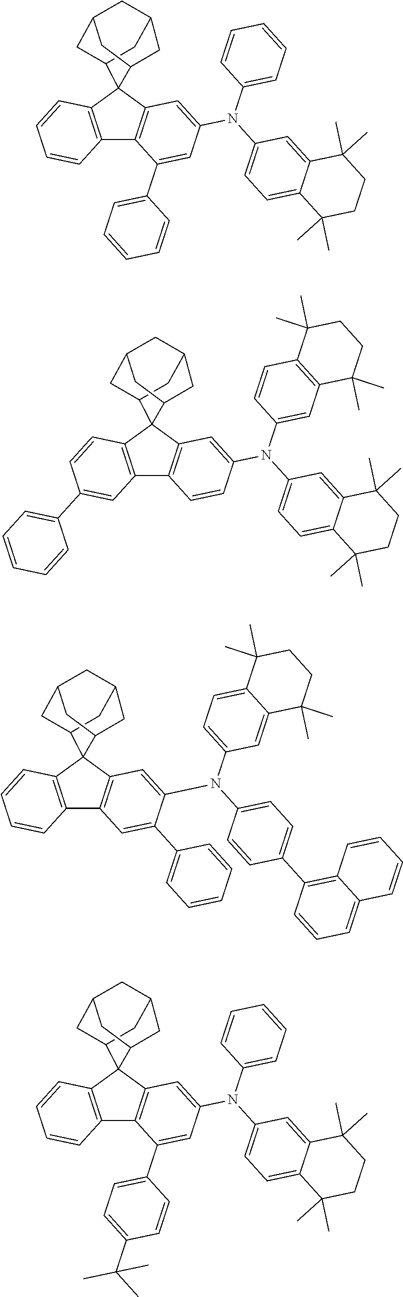 [SMILES: CC1(C)CCC(C)(C)c2cc(N(c3ccccc3)c3cc(-c4ccccc4)c4c(c3)C3(c5ccccc5-4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5ccc(-c6ccccc6)cc5-4)C4CC5CC(C4)CC3C5)ccc21 ; CC1(C)CCC(C)(C)c2cc(N(c3ccc(-c4cccc5ccccc45)cc3)c3cc4c(cc3-c3ccccc3)-c3ccccc3C43C4CC5CC(C4)CC3C5)ccc21 ; CC(C)(C)c1ccc(-c2cc(N(c3ccccc3)c3ccc4c(c3)C(C)(C)CCC4(C)C)cc3c2-c2ccccc2C32C3CC4CC(C3)CC2C4)cc1]

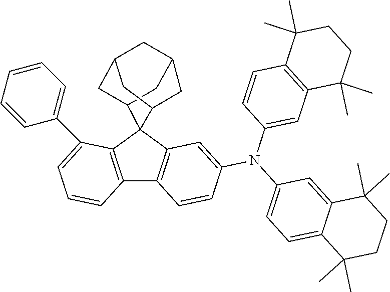 [SMILES: CC1(C)CCC(C)(C)c2cc(N(c3ccc4c(c3)C(C)(C)CCC4(C)C)c3ccc4c(c3)C3(c5c(-c6ccccc6)cccc5-4)C4CC5CC(C4)CC3C5)ccc21]

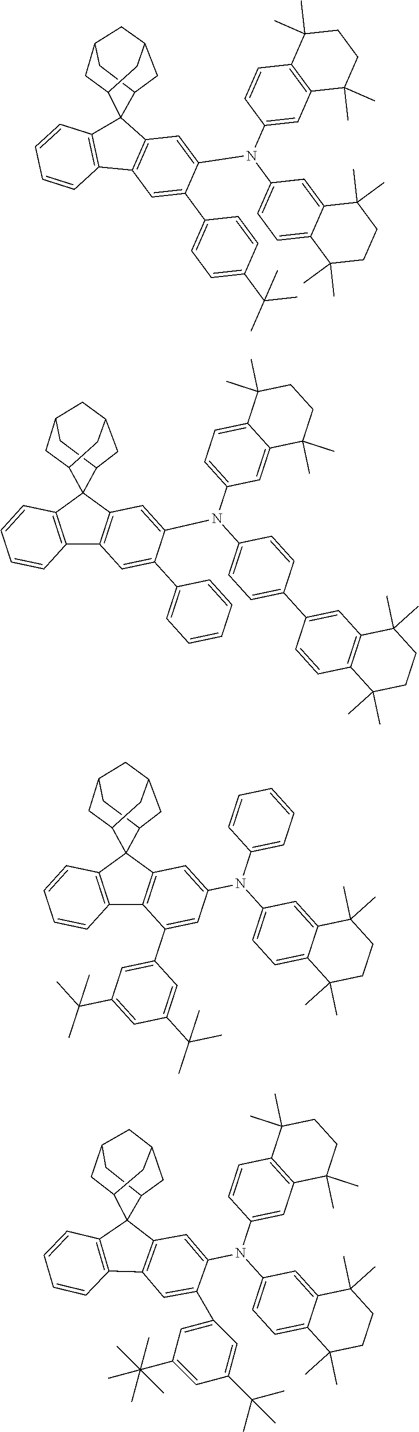

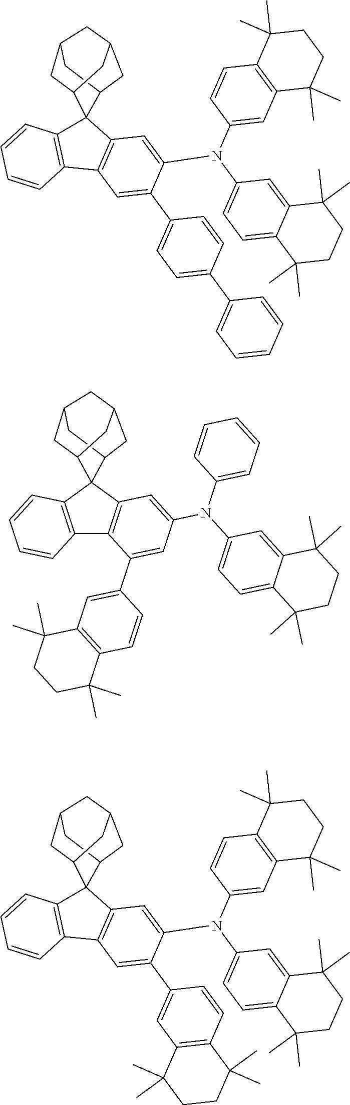 [SMILES: CC1(C)CCC(C)(C)c2cc(N(c3ccc4c(c3)C(C)(C)CCC4(C)C)c3cc4c(cc3-c3ccc(-c5ccccc5)cc3)-c3ccccc3C43C4CC5CC(C4)CC3C5)ccc21 ; CC1(C)CCC(C)(C)c2cc(-c3cc(N(c4ccccc4)c4ccc5c(c4)C(C)(C)CCC5(C)C)cc4c3-c3ccccc3C43C4CC5CC(C4)CC3C5)ccc21 ; CC1(C)CCC(C)(C)c2cc(-c3cc4c(cc3N(c3ccc5c(c3)C(C)(C)CCC5(C)C)c3ccc5c(c3)C(C)(C)CCC5(C)C)C3(c5ccccc5-4)C4CC5CC(C4)CC3C5)ccc21]

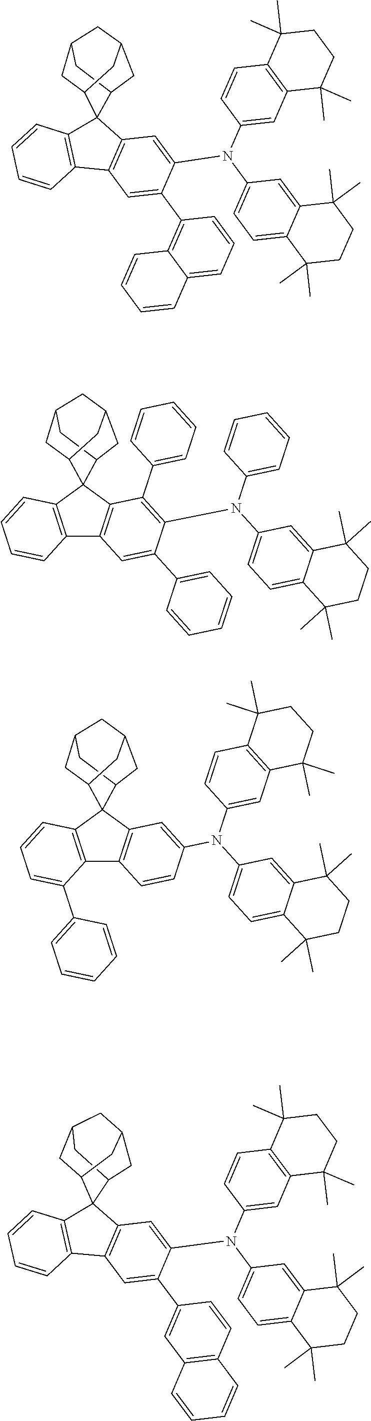 [SMILES: CC1(C)CCC(C)(C)c2cc(N(c3ccc4c(c3)C(C)(C)CCC4(C)C)c3cc4c(cc3-c3cccc5ccccc35)-c3ccccc3C43C4CC5CC(C4)CC3C5)ccc21 ; CC1(C)CCC(C)(C)c2cc(N(c3ccccc3)c3c(-c4ccccc4)cc4c(c3-c3ccccc3)C3(c5ccccc5-4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5cccc(-c6ccccc6)c5-4)C4CC5CC(C4)CC3C5)ccc21 ; CC1(C)CCC(C)(C)c2cc(N(c3ccc4c(c3)C(C)(C)CCC4(C)C)c3cc4c(cc3-c3ccc5ccccc5c3)-c3ccccc3C43C4CC5CC(C4)CC3C5)ccc21]

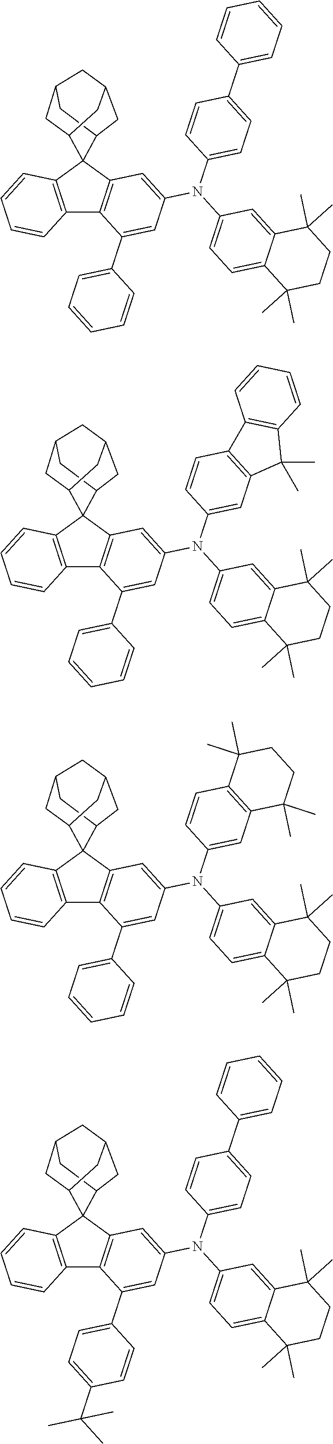 [SMILES: CC1(C)CCC(C)(C)c2cc(N(c3ccc(-c4ccccc4)cc3)c3cc(-c4ccccc4)c4c(c3)C3(c5ccccc5-4)C4CC5CC(C4)CC3C5)ccc21 ; CC1(C)CCC(C)(C)c2cc(N(c3ccc4c(c3)C(C)(C)c3ccccc3-4)c3cc(-c4ccccc4)c4c(c3)C3(c5ccccc5-4)C4CC5CC(C4)CC3C5)ccc21 ; CC1(C)CCC(C)(C)c2cc(N(c3ccc4c(c3)C(C)(C)CCC4(C)C)c3cc(-c4ccccc4)c4c(c3)C3(c5ccccc5-4)C4CC5CC(C4)CC3C5)ccc21 ; CC(C)(C)c1ccc(-c2cc(N(c3ccc(-c4ccccc4)cc3)c3ccc4c(c3)C(C)(C)CCC4(C)C)cc3c2-c2ccccc2C32C3CC4CC(C3)CC2C4)cc1]

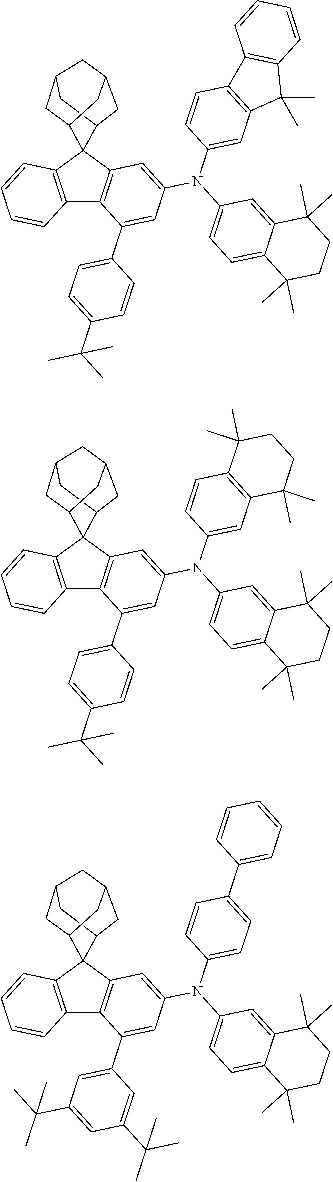 [SMILES: CC(C)(C)c1ccc(-c2cc(N(c3ccc4c(c3)C(C)(C)CCC4(C)C)c3ccc4c(c3)C(C)(C)c3ccccc3-4)cc3c2-c2ccccc2C32C3CC4CC(C3)CC2C4)cc1 ; CC(C)(C)c1ccc(-c2cc(N(c3ccc4c(c3)C(C)(C)CCC4(C)C)c3ccc4c(c3)C(C)(C)CCC4(C)C)cc3c2-c2ccccc2C32C3CC4CC(C3)CC2C4)cc1 ; CC(C)(C)c1cc(-c2cc(N(c3ccc(-c4ccccc4)cc3)c3ccc4c(c3)C(C)(C)CCC4(C)C)cc3c2-c2ccccc2C32C3CC4CC(C3)CC2C4)cc(C(C)(C)C)c1]

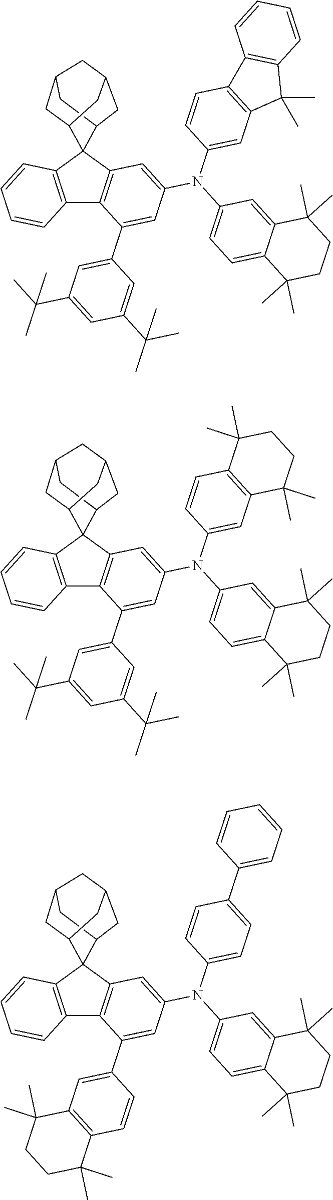 [SMILES: CC(C)(C)c1cc(-c2cc(N(c3ccc4c(c3)C(C)(C)CCC4(C)C)c3ccc4c(c3)C(C)(C)c3ccccc3-4)cc3c2-c2ccccc2C32C3CC4CC(C3)CC2C4)cc(C(C)(C)C)c1 ; CC(C)(C)c1cc(-c2cc(N(c3ccc4c(c3)C(C)(C)CCC4(C)C)c3ccc4c(c3)C(C)(C)CCC4(C)C)cc3c2-c2ccccc2C32C3CC4CC(C3)CC2C4)cc(C(C)(C)C)c1 ; CC1(C)CCC(C)(C)c2cc(-c3cc(N(c4ccc(-c5ccccc5)cc4)c4ccc5c(c4)C(C)(C)CCC5(C)C)cc4c3-c3ccccc3C43C4CC5CC(C4)CC3C5)ccc21]

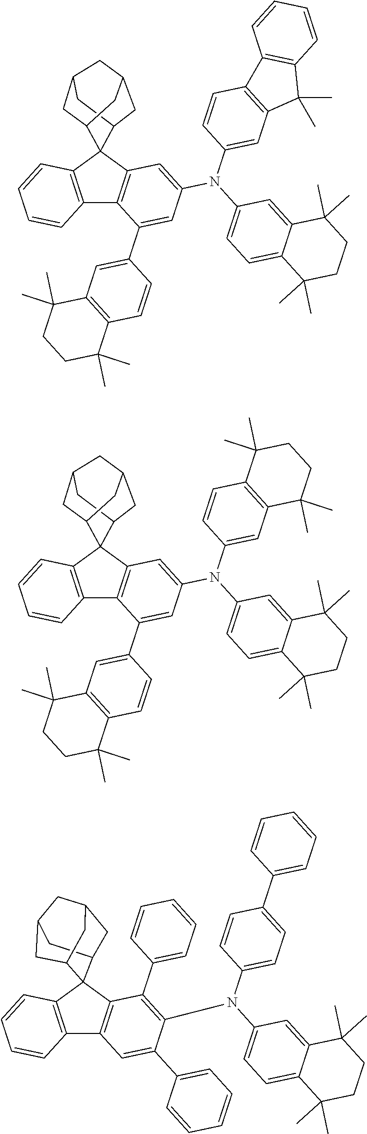 [SMILES: CC1(C)CCC(C)(C)c2cc(-c3cc(N(c4ccc5c(c4)C(C)(C)CCC5(C)C)c4ccc5c(c4)C(C)(C)c4ccccc4-5)cc4c3-c3ccccc3C43C4CC5CC(C4)CC3C5)ccc21 ; CC1(C)CCC(C)(C)c2cc(-c3cc(N(c4ccc5c(c4)C(C)(C)CCC5(C)C)c4ccc5c(c4)C(C)(C)CCC5(C)C)cc4c3-c3ccccc3C43C4CC5CC(C4)CC3C5)ccc21 ; CC1(C)CCC(C)(C)c2cc(N(c3ccc(-c4ccccc4)cc3)c3c(-c4ccccc4)cc4c(c3-c3ccccc3)C3(c5ccccc5-4)C4CC5CC(C4)CC3C5)ccc21]

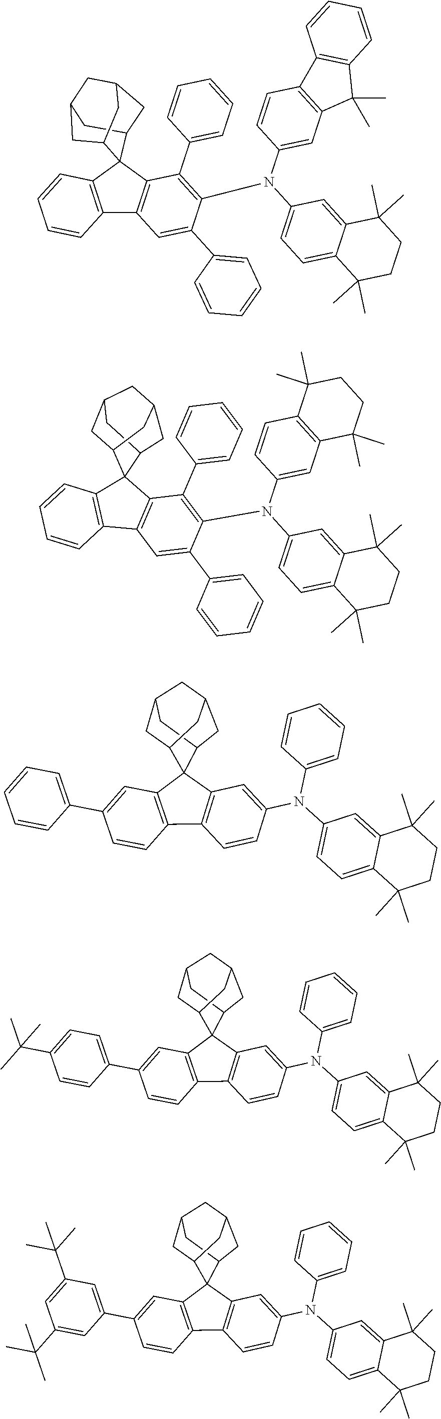

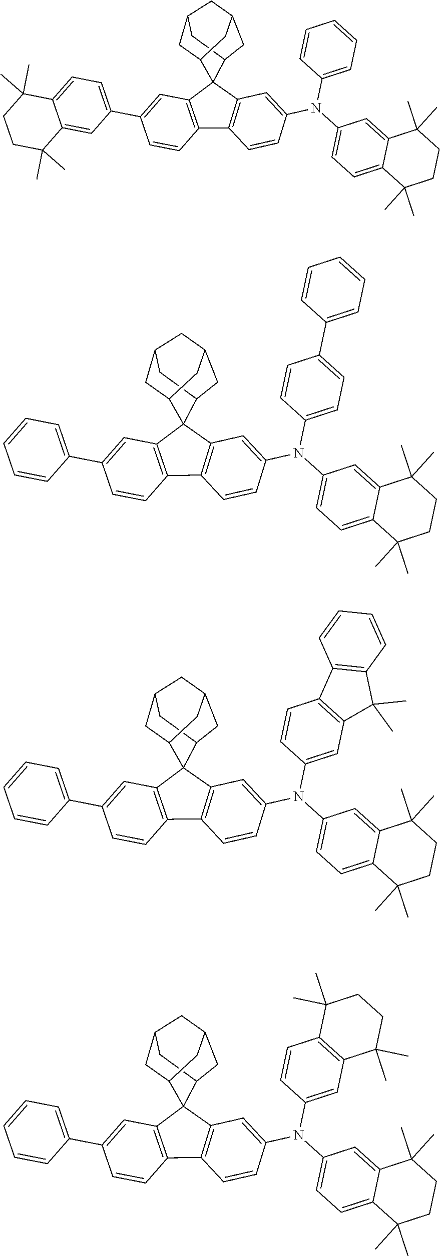 [SMILES: CC1(C)CCC(C)(C)c2cc(-c3ccc4c(c3)C3(c5cc(N(c6ccccc6)c6ccc7c(c6)C(C)(C)CCC7(C)C)ccc5-4)C4CC5CC(C4)CC3C5)ccc21 ; CC1(C)CCC(C)(C)c2cc(N(c3ccc(-c4ccccc4)cc3)c3ccc4c(c3)C3(c5cc(-c6ccccc6)ccc5-4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5cc(-c6ccccc6)ccc5-4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5cc(-c6ccccc6)ccc5-4)C4CC5CC(C4)CC3C5)ccc21]

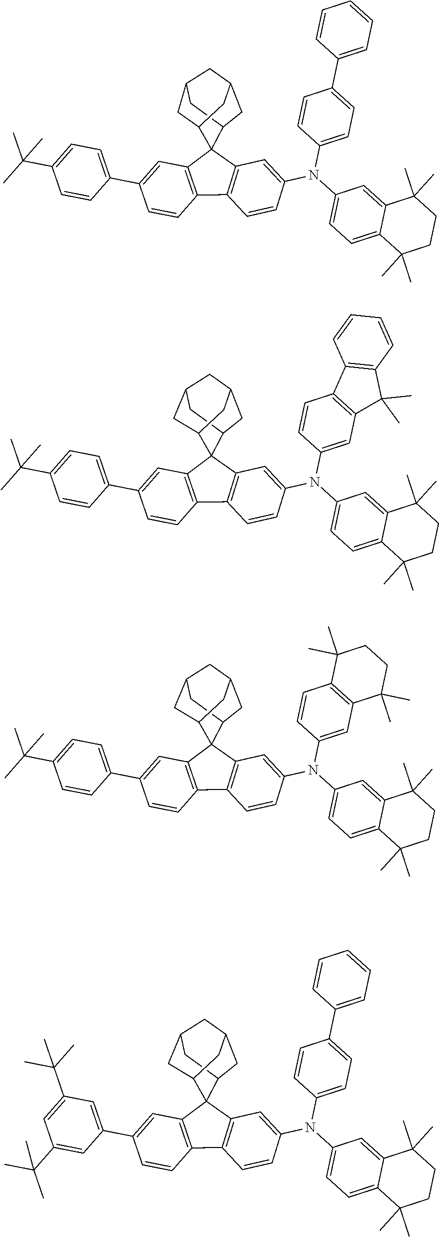 [SMILES: CC(C)(C)c1ccc(-c2ccc3c(c2)C2(c4cc(N(c5ccc(-c6ccccc6)cc5)c5ccc6c(c5)C(C)(C)CCC6(C)C)ccc4-3)C3CC4CC(C3)CC2C4)cc1 ; CC(C)(C)c1ccc(-c2ccc3c(c2)C2(c4cc(N(c5ccc6c(c5)C(C)(C)CCC6(C)C)c5ccc6c(c5)C(C)(C)c5ccccc5-6)ccc4-3)C3CC4CC(C3)CC2C4)cc1 ; CC(C)(C)c1ccc(-c2ccc3c(c2)C2(c4cc(N(c5ccc6c(c5)C(C)(C)CCC6(C)C)c5ccc6c(c5)C(C)(C)CCC6(C)C)ccc4-3)C3CC4CC(C3)CC2C4)cc1 ; CC(C)(C)c1cc(-c2ccc3c(c2)C2(c4cc(N(c5ccc(-c6ccccc6)cc5)c5ccc6c(c5)C(C)(C)CCC6(C)C)ccc4-3)C3CC4CC(C3)CC2C4)cc(C(C)(C)C)c1]

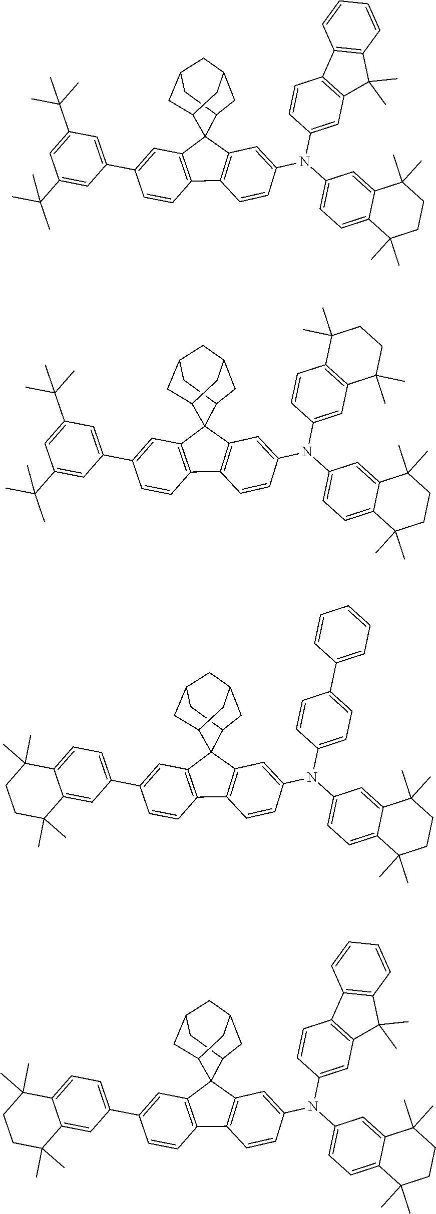

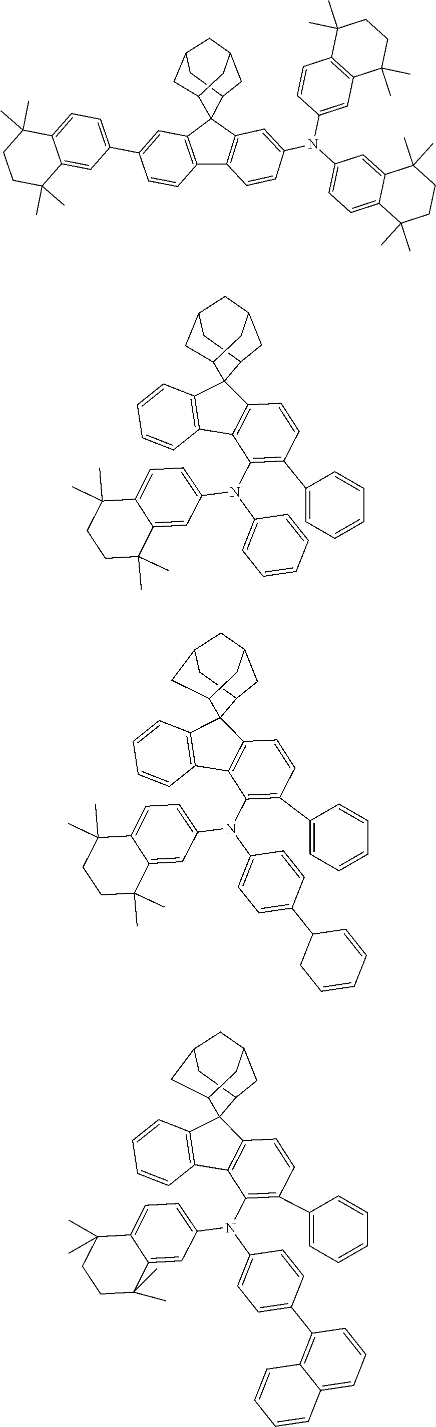 [SMILES: CC1(C)CCC(C)(C)c2cc(-c3ccc4c(c3)C3(c5cc(N(c6ccc7c(c6)C(C)(C)CCC7(C)C)c6ccc7c(c6)C(C)(C)CCC7(C)C)ccc5-4)C4CC5CC(C4)CC3C5)ccc21 ; CC1(C)CCC(C)(C)c2cc(N(c3ccccc3)c3c(-c4ccccc4)ccc4c3-c3ccccc3C43C4CC5CC(C4)CC3C5)ccc21 ; CC1(C)CCC(C)(C)c2cc(N(c3ccc(C4C=CC=CC4)cc3)c3c(-c4ccccc4)ccc4c3-c3ccccc3C43C4CC5CC(C4)CC3C5)ccc21 ; CC1(C)CCC(C)(C)c2cc(N(c3ccc(-c4cccc5ccccc45)cc3)c3c(-c4ccccc4)ccc4c3-c3ccccc3C43C4CC5CC(C4)CC3C5)ccc21]

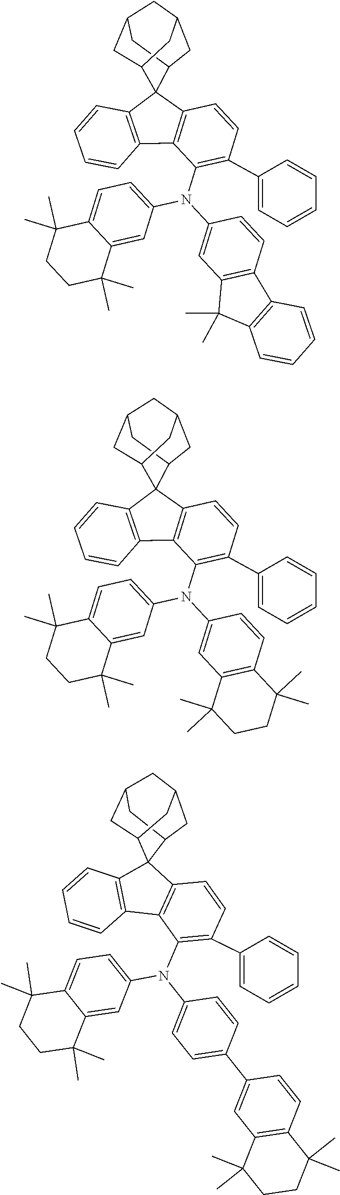

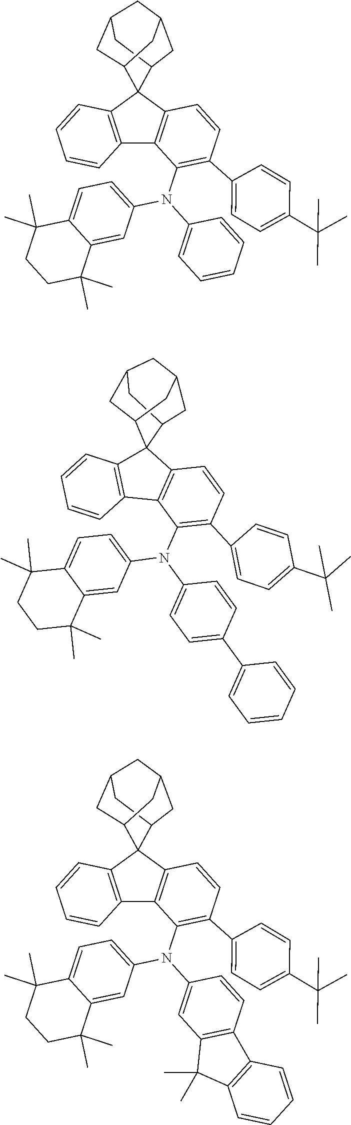

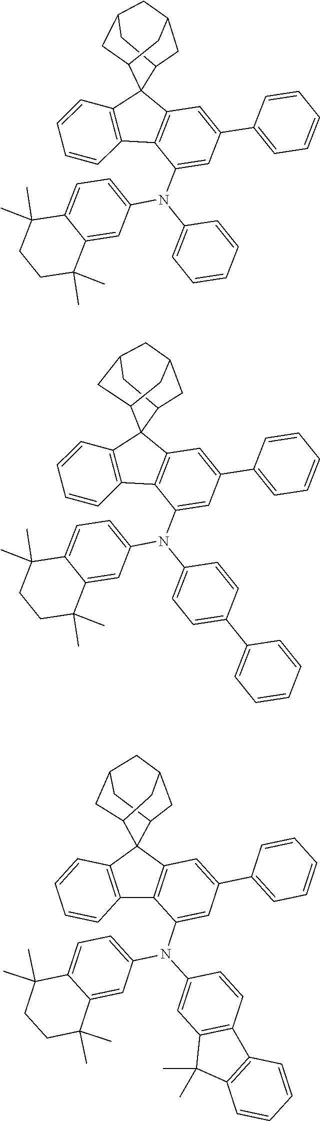 [SMILES: CC1(C)CCC(C)(C)c2cc(N(c3ccccc3)c3cc(-c4ccccc4)cc4c3-c3ccccc3C43C4CC5CC(C4)CC3C5)ccc21 ; CC1(C)CCC(C)(C)c2cc(N(c3ccc(-c4ccccc4)cc3)c3cc(-c4ccccc4)cc4c3-c3ccccc3C43C4CC5CC(C4)CC3C5)ccc21 ; CC1(C)CCC(C)(C)c2cc(N(c3ccc4c(c3)C(C)(C)c3ccccc3-4)c3cc(-c4ccccc4)cc4c3-c3ccccc3C43C4CC5CC(C4)CC3C5)ccc21]

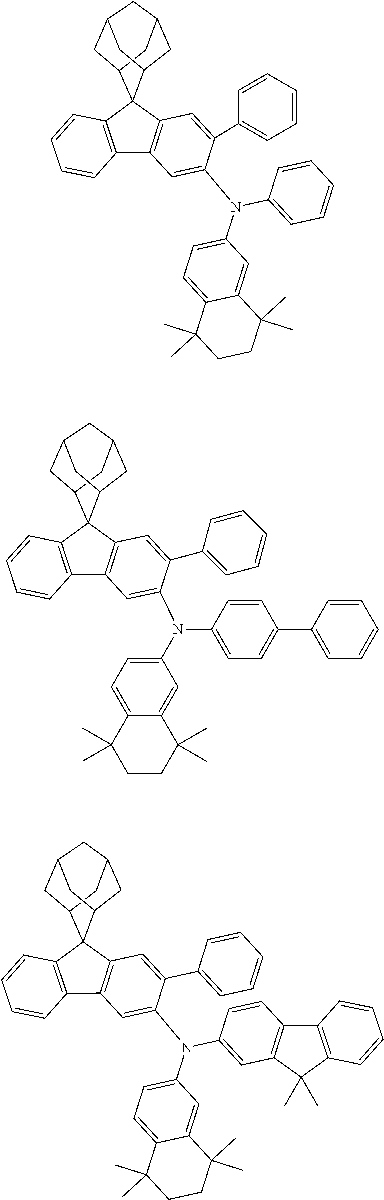 [SMILES: CC1(C)CCC(C)(C)c2cc(N(c3ccccc3)c3cc4c(cc3-c3ccccc3)C3(c5ccccc5-4)C4CC5CC(C4)CC3C5)ccc21 ; CC1(C)CCC(C)(C)c2cc(N(c3ccc(-c4ccccc4)cc3)c3cc4c(cc3-c3ccccc3)C3(c5ccccc5-4)C4CC5CC(C4)CC3C5)ccc21 ; CC1(C)CCC(C)(C)c2cc(N(c3ccc4c(c3)C(C)(C)c3ccccc3-4)c3cc4c(cc3-c3ccccc3)C3(c5ccccc5-4)C4CC5CC(C4)CC3C5)ccc21]

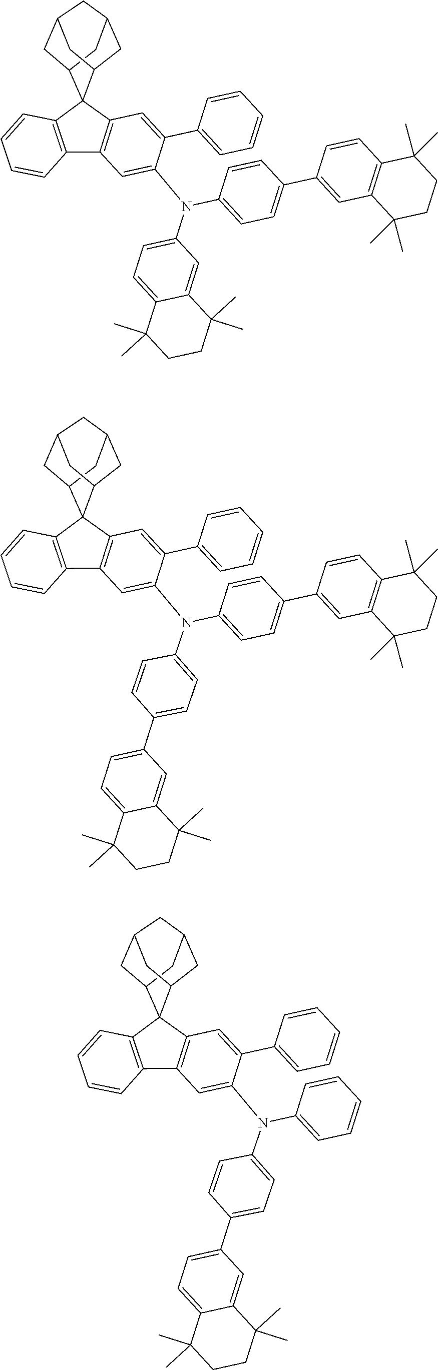 [SMILES: CC1(C)CCC(C)(C)c2cc(-c3ccc(N(c4ccc5c(c4)C(C)(C)CCC5(C)C)c4cc5c(cc4-c4ccccc4)C4(c6ccccc6-5)C5CC6CC(C5)CC4C6)cc3)ccc21 ; CC1(C)CCC(C)(C)c2cc(-c3ccc(N(c4ccc(-c5ccc6c(c5)C(C)(C)CCC6(C)C)cc4)c4cc5c(cc4-c4ccccc4)C4(c6ccccc6-5)C5CC6CC(C5)CC4C6)cc3)ccc21 ; CC1(C)CCC(C)(C)c2cc(-c3ccc(N(c4ccccc4)c4cc5c(cc4-c4ccccc4)C4(c6ccccc6-5)C5CC6CC(C5)CC4C6)cc3)ccc21]

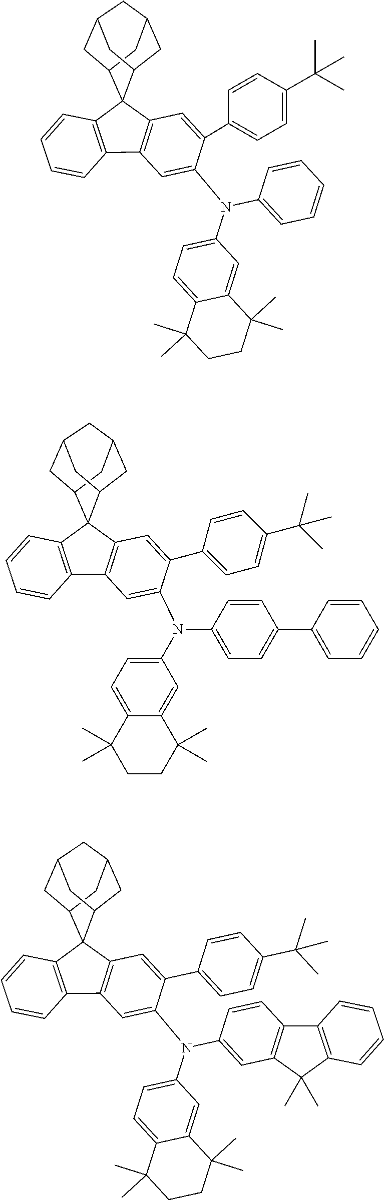 [SMILES: CC(C)(C)c1ccc(-c2cc3c(cc2N(c2ccccc2)c2ccc4c(c2)C(C)(C)CCC4(C)C)-c2ccccc2C32C3CC4CC(C3)CC2C4)cc1 ; CC(C)(C)c1ccc(-c2cc3c(cc2N(c2ccc(-c4ccccc4)cc2)c2ccc4c(c2)C(C)(C)CCC4(C)C)-c2ccccc2C32C3CC4CC(C3)CC2C4)cc1 ; CC(C)(C)c1ccc(-c2cc3c(cc2N(c2ccc4c(c2)C(C)(C)CCC4(C)C)c2ccc4c(c2)C(C)(C)c2ccccc2-4)-c2ccccc2C32C3CC4CC(C3)CC2C4)cc1]

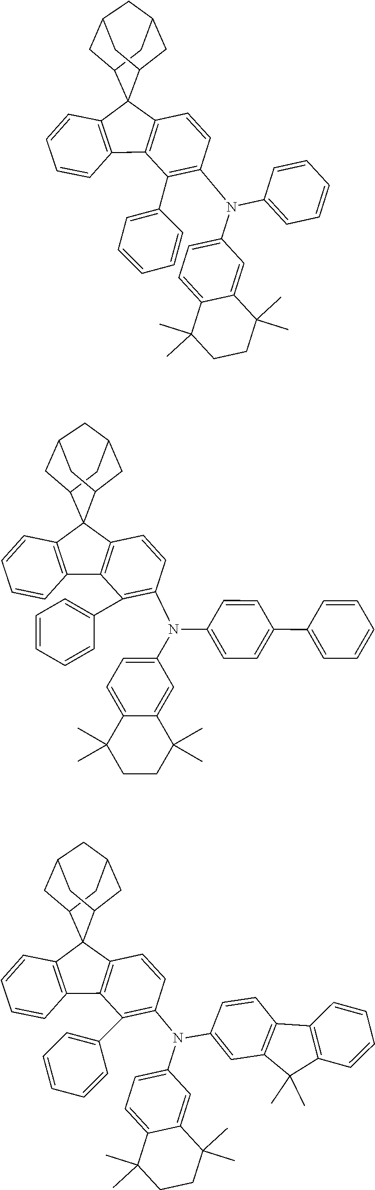 [SMILES: CC1(C)CCC(C)(C)c2cc(N(c3ccccc3)c3ccc4c(c3-c3ccccc3)-c3ccccc3C43C4CC5CC(C4)CC3C5)ccc21 ; CC1(C)CCC(C)(C)c2cc(N(c3ccc(-c4ccccc4)cc3)c3ccc4c(c3-c3ccccc3)-c3ccccc3C43C4CC5CC(C4)CC3C5)ccc21 ; CC1(C)CCC(C)(C)c2cc(N(c3ccc4c(c3)C(C)(C)c3ccccc3-4)c3ccc4c(c3-c3ccccc3)-c3ccccc3C43C4CC5CC(C4)CC3C5)ccc21]

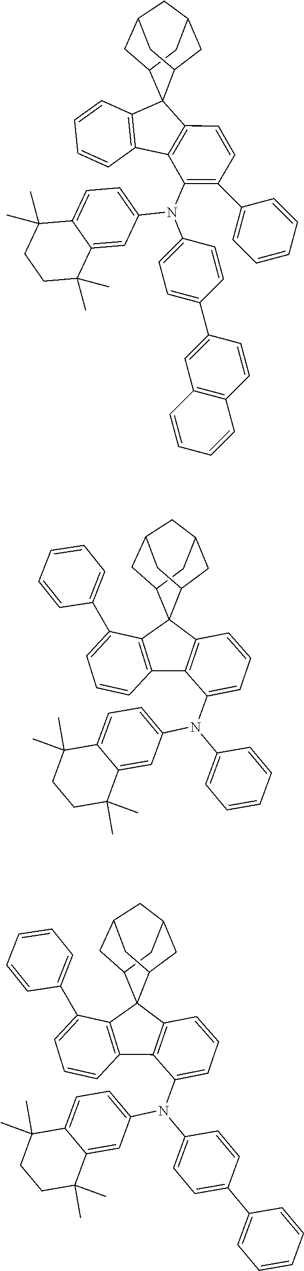 [SMILES: CC1(C)CCC(C)(C)c2cc(N(c3ccc(-c4ccc5ccccc5c4)cc3)c3c(-c4ccccc4)ccc4c3-c3ccccc3C43C4CC5CC(C4)CC3C5)ccc21 ; CC1(C)CCC(C)(C)c2cc(N(c3ccccc3)c3cccc4c3-c3cccc(-c5ccccc5)c3C43C4CC5CC(C4)CC3C5)ccc21 ; CC1(C)CCC(C)(C)c2cc(N(c3ccc(-c4ccccc4)cc3)c3cccc4c3-c3cccc(-c5ccccc5)c3C43C4CC5CC(C4)CC3C5)ccc21]

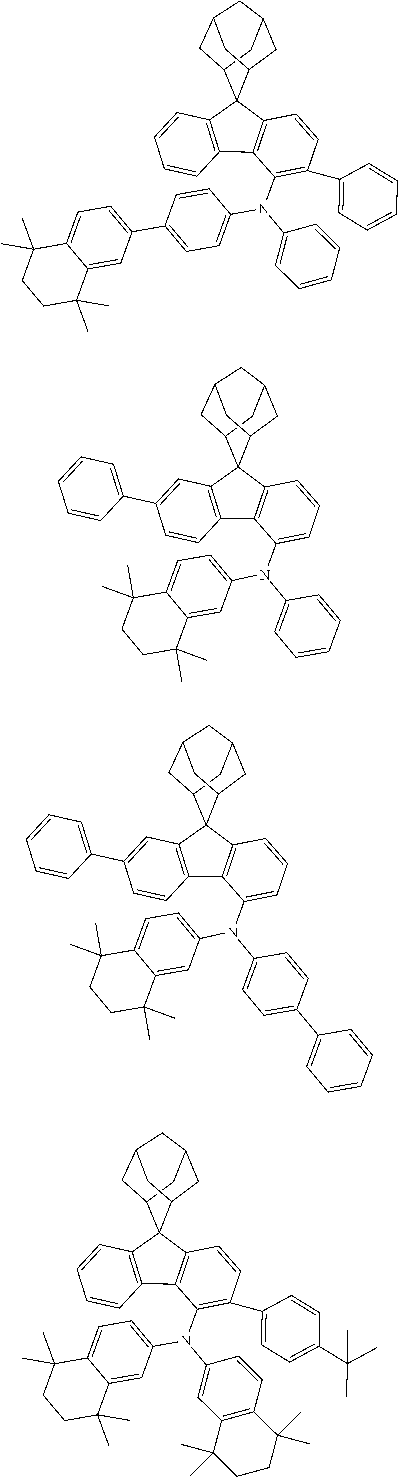 [SMILES: CC1(C)CCC(C)(C)c2cc(-c3ccc(N(c4ccccc4)c4c(-c5ccccc5)ccc5c4-c4ccccc4C54C5CC6CC(C5)CC4C6)cc3)ccc21 ; CC1(C)CCC(C)(C)c2cc(N(c3ccccc3)c3cccc4c3-c3ccc(-c5ccccc5)cc3C43C4CC5CC(C4)CC3C5)ccc21 ; CC1(C)CCC(C)(C)c2cc(N(c3ccc(-c4ccccc4)cc3)c3cccc4c3-c3ccc(-c5ccccc5)cc3C43C4CC5CC(C4)CC3C5)ccc21 ; CC(C)(C)c1ccc(-c2ccc3c(c2N(c2ccc4c(c2)C(C)(C)CCC4(C)C)c2ccc4c(c2)C(C)(C)CCC4(C)C)-c2ccccc2C32C3CC4CC(C3)CC2C4)cc1]

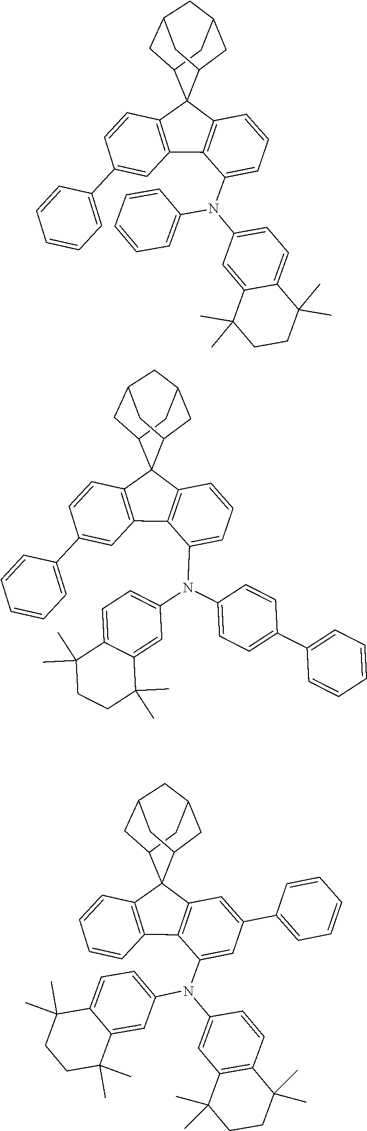

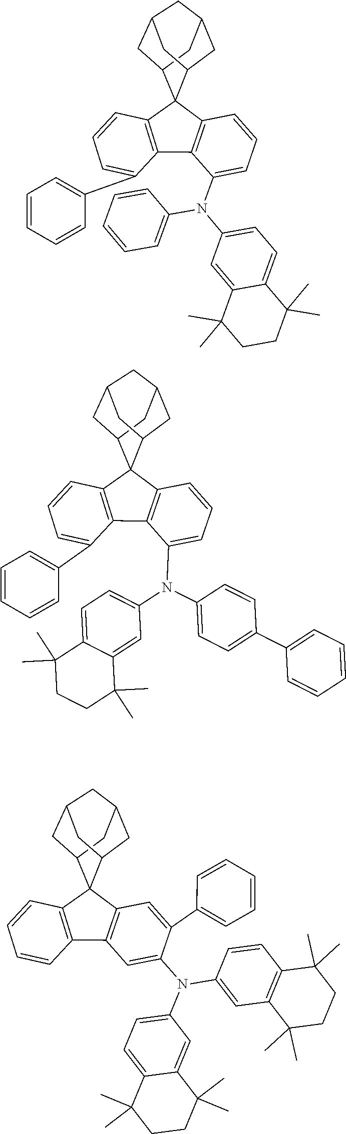 [SMILES: CC1(C)CCC(C)(C)c2cc(N(c3ccccc3)c3cccc4c3-c3c(-c5ccccc5)cccc3C43C4CC5CC(C4)CC3C5)ccc21 ; CC1(C)CCC(C)(C)c2cc(N(c3ccc(-c4ccccc4)cc3)c3cccc4c3-c3c(-c5ccccc5)cccc3C43C4CC5CC(C4)CC3C5)ccc21 ; CC1(C)CCC(C)(C)c2cc(N(c3ccc4c(c3)C(C)(C)CCC4(C)C)c3cc4c(cc3-c3ccccc3)C3(c5ccccc5-4)C4CC5CC(C4)CC3C5)ccc21]

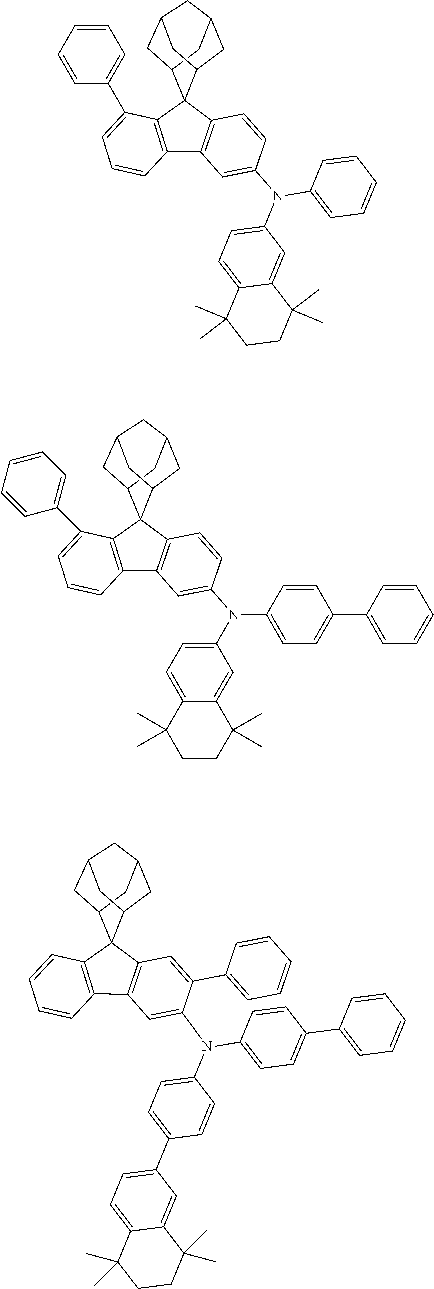 [SMILES: CC1(C)CCC(C)(C)c2cc(N(c3ccccc3)c3ccc4c(c3)-c3cccc(-c5ccccc5)c3C43C4CC5CC(C4)CC3C5)ccc21 ; CC1(C)CCC(C)(C)c2cc(N(c3ccc(-c4ccccc4)cc3)c3ccc4c(c3)-c3cccc(-c5ccccc5)c3C43C4CC5CC(C4)CC3C5)ccc21 ; CC1(C)CCC(C)(C)c2cc(-c3ccc(N(c4ccc(-c5ccccc5)cc4)c4cc5c(cc4-c4ccccc4)C4(c6ccccc6-5)C5CC6CC(C5)CC4C6)cc3)ccc21]

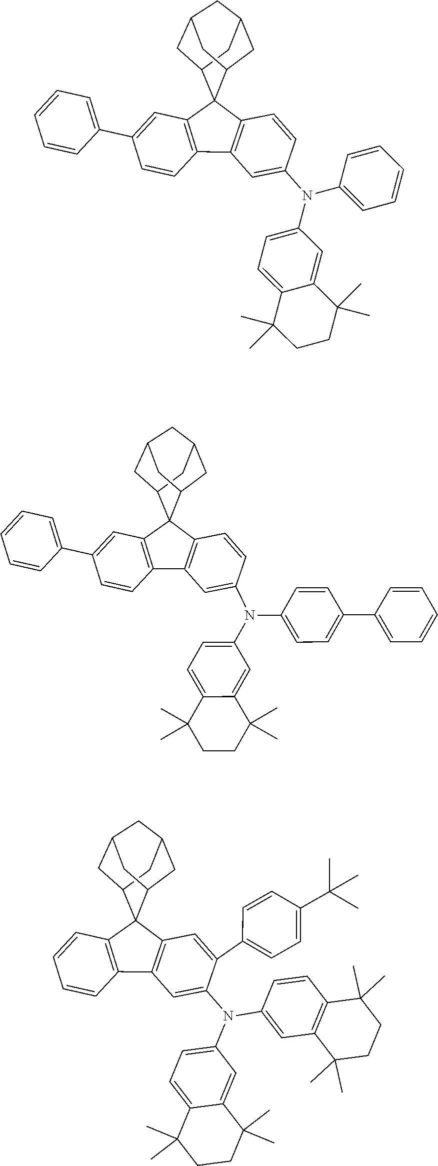 [SMILES: CC1(C)CCC(C)(C)c2cc(N(c3ccccc3)c3ccc4c(c3)-c3ccc(-c5ccccc5)cc3C43C4CC5CC(C4)CC3C5)ccc21 ; CC1(C)CCC(C)(C)c2cc(N(c3ccc(-c4ccccc4)cc3)c3ccc4c(c3)-c3ccc(-c5ccccc5)cc3C43C4CC5CC(C4)CC3C5)ccc21 ; CC(C)(C)c1ccc(-c2cc3c(cc2N(c2ccc4c(c2)C(C)(C)CCC4(C)C)c2ccc4c(c2)C(C)(C)CCC4(C)C)-c2ccccc2C32C3CC4CC(C3)CC2C4)cc1]

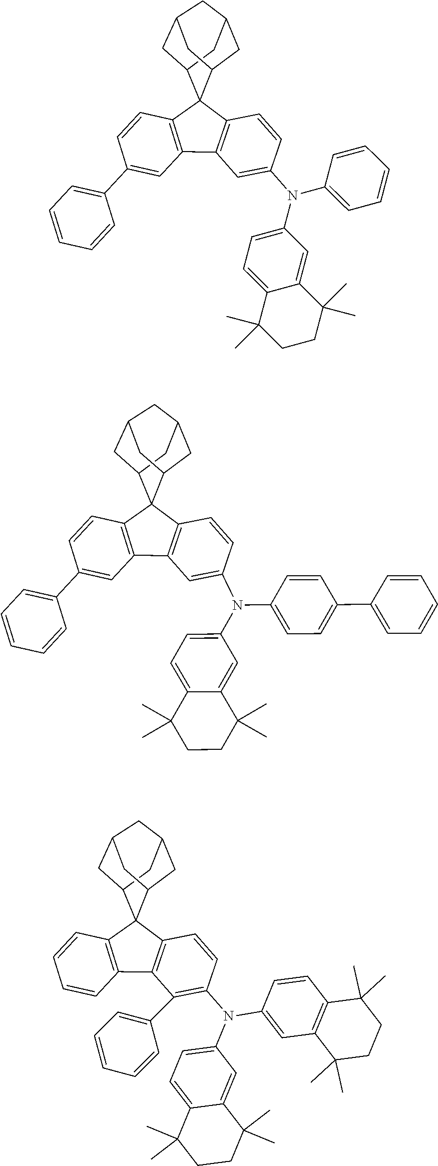 [SMILES: CC1(C)CCC(C)(C)c2cc(N(c3ccccc3)c3ccc4c(c3)-c3cc(-c5ccccc5)ccc3C43C4CC5CC(C4)CC3C5)ccc21 ; CC1(C)CCC(C)(C)c2cc(N(c3ccc(-c4ccccc4)cc3)c3ccc4c(c3)-c3cc(-c5ccccc5)ccc3C43C4CC5CC(C4)CC3C5)ccc21 ; CC1(C)CCC(C)(C)c2cc(N(c3ccc4c(c3)C(C)(C)CCC4(C)C)c3ccc4c(c3-c3ccccc3)-c3ccccc3C43C4CC5CC(C4)CC3C5)ccc21]

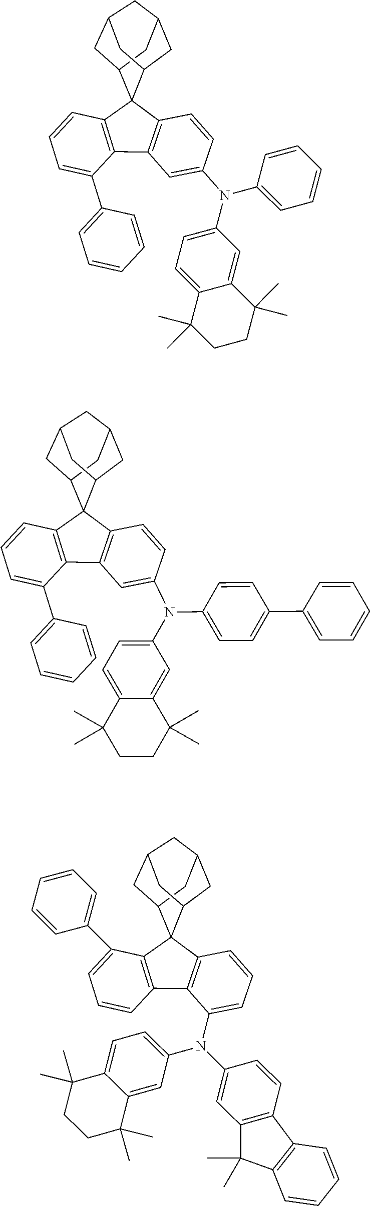 [SMILES: CC1(C)CCC(C)(C)c2cc(N(c3ccccc3)c3ccc4c(c3)-c3c(-c5ccccc5)cccc3C43C4CC5CC(C4)CC3C5)ccc21 ; CC1(C)CCC(C)(C)c2cc(N(c3ccc(-c4ccccc4)cc3)c3ccc4c(c3)-c3c(-c5ccccc5)cccc3C43C4CC5CC(C4)CC3C5)ccc21 ; CC1(C)CCC(C)(C)c2cc(N(c3ccc4c(c3)C(C)(C)c3ccccc3-4)c3cccc4c3-c3cccc(-c5ccccc5)c3C43C4CC5CC(C4)CC3C5)ccc21]

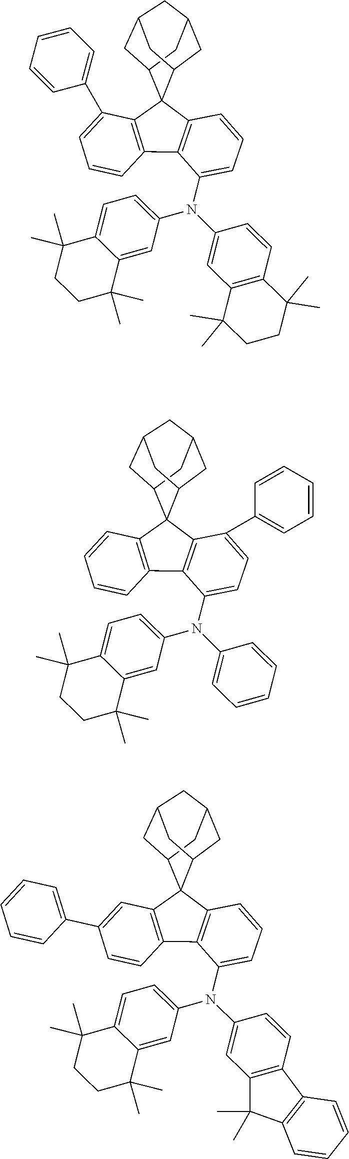

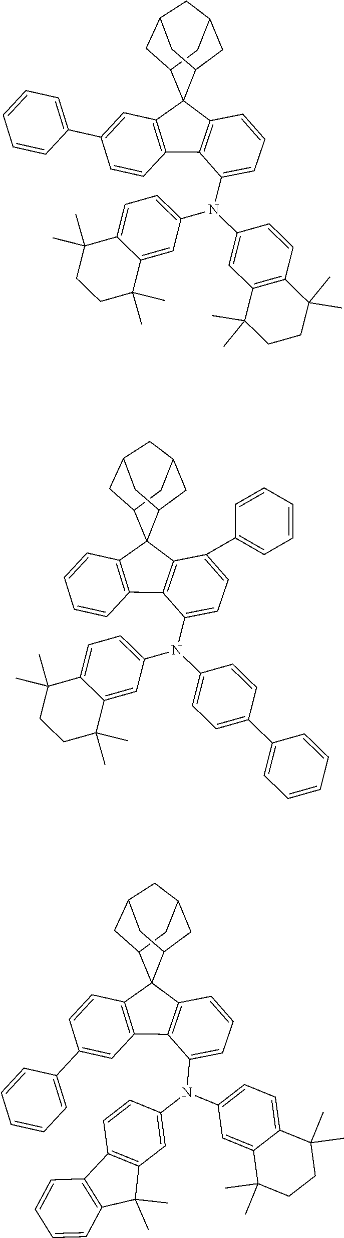

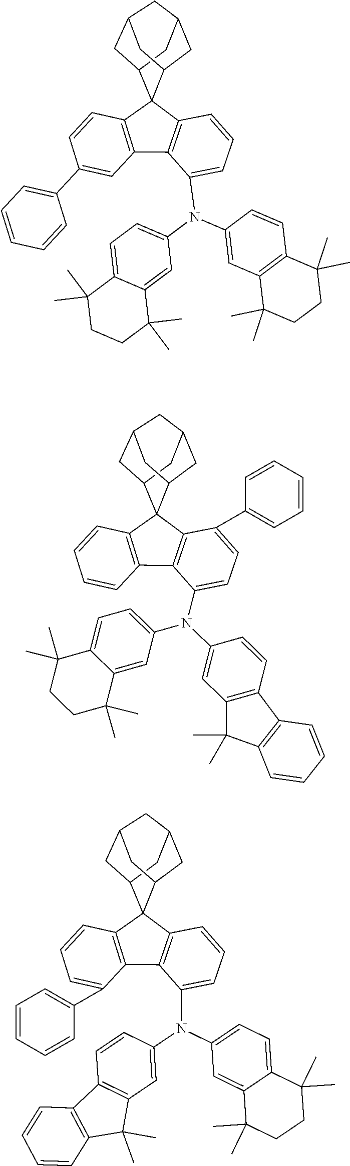

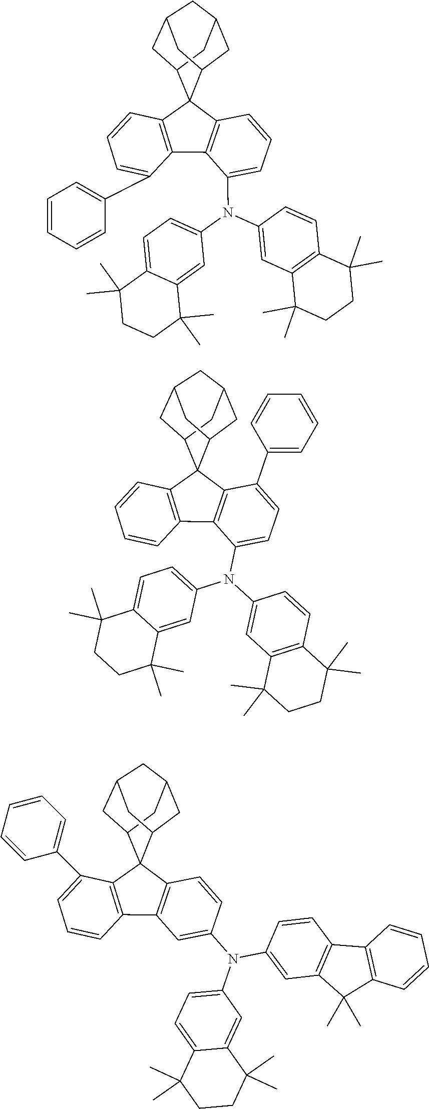

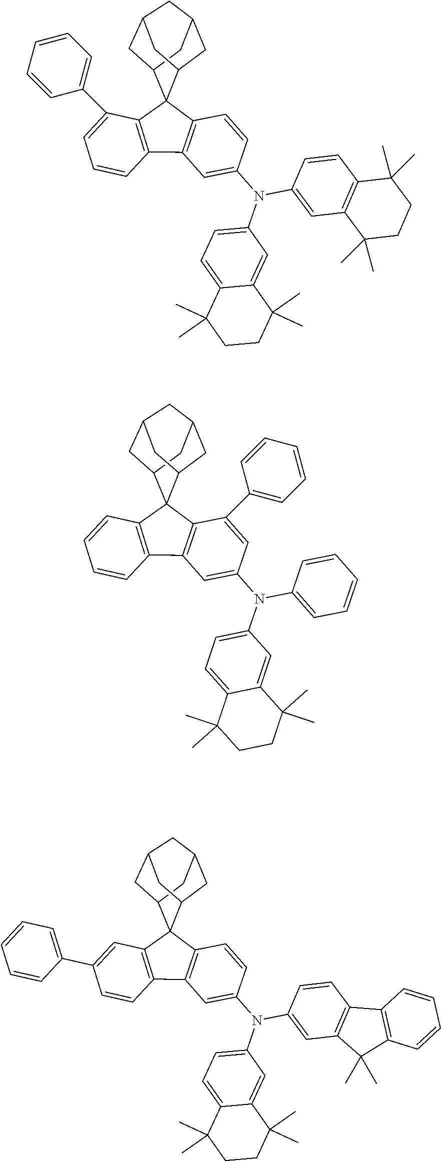 [SMILES: CC1(C)CCC(C)(C)c2cc(N(c3ccc4c(c3)-c3cccc(-c5ccccc5)c3C43C4CC5CC(C4)CC3C5)c3ccc4c(c3)C(C)(C)CCC4(C)C)ccc21 ; CC1(C)CCC(C)(C)c2cc(N(c3ccccc3)c3cc(-c4ccccc4)c4c(c3)-c3ccccc3C43C4CC5CC(C4)CC3C5)ccc21 ; CC1(C)CCC(C)(C)c2cc(N(c3ccc4c(c3)-c3ccc(-c5ccccc5)cc3C43C4CC5CC(C4)CC3C5)c3ccc4c(c3)C(C)(C)c3ccccc3-4)ccc21]

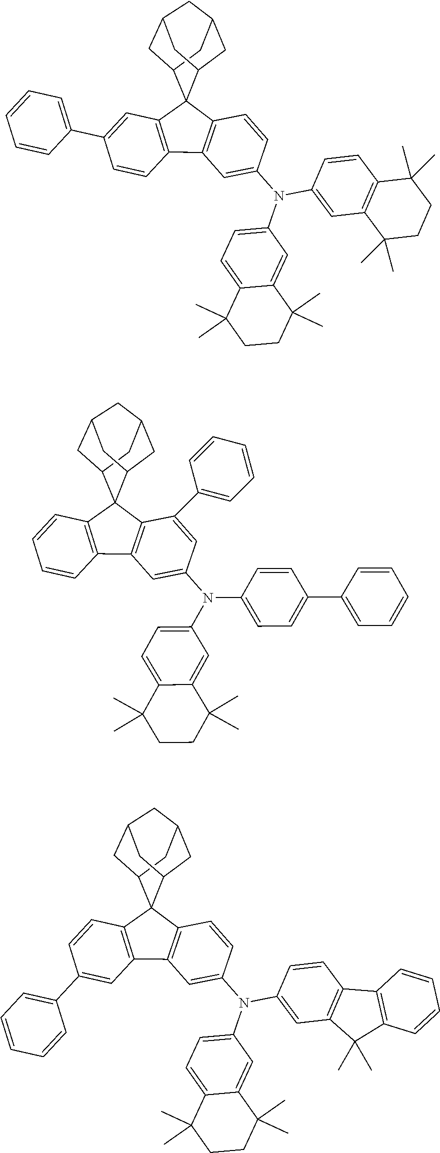 [SMILES: CC1(C)CCC(C)(C)c2cc(N(c3ccc4c(c3)-c3ccc(-c5ccccc5)cc3C43C4CC5CC(C4)CC3C5)c3ccc4c(c3)C(C)(C)CCC4(C)C)ccc21 ; CC1(C)CCC(C)(C)c2cc(N(c3ccc(-c4ccccc4)cc3)c3cc(-c4ccccc4)c4c(c3)-c3ccccc3C43C4CC5CC(C4)CC3C5)ccc21 ; CC1(C)CCC(C)(C)c2cc(N(c3ccc4c(c3)-c3cc(-c5ccccc5)ccc3C43C4CC5CC(C4)CC3C5)c3ccc4c(c3)C(C)(C)c3ccccc3-4)ccc21]

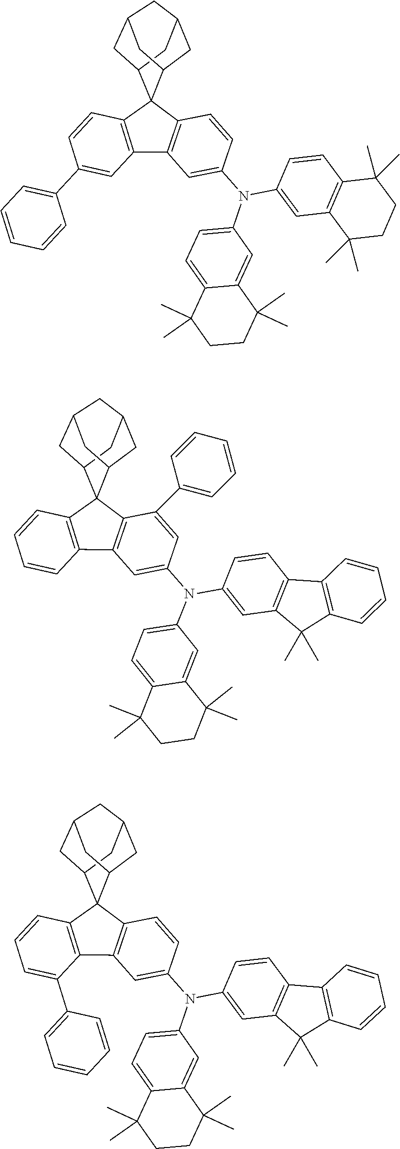 [SMILES: CC1(C)CCC(C)(C)c2cc(N(c3ccc4c(c3)-c3cc(-c5ccccc5)ccc3C43C4CC5CC(C4)CC3C5)c3ccc4c(c3)C(C)(C)CCC4(C)C)ccc21 ; CC1(C)CCC(C)(C)c2cc(N(c3cc(-c4ccccc4)c4c(c3)-c3ccccc3C43C4CC5CC(C4)CC3C5)c3ccc4c(c3)C(C)(C)c3ccccc3-4)ccc21 ; CC1(C)CCC(C)(C)c2cc(N(c3ccc4c(c3)-c3c(-c5ccccc5)cccc3C43C4CC5CC(C4)CC3C5)c3ccc4c(c3)C(C)(C)c3ccccc3-4)ccc21]

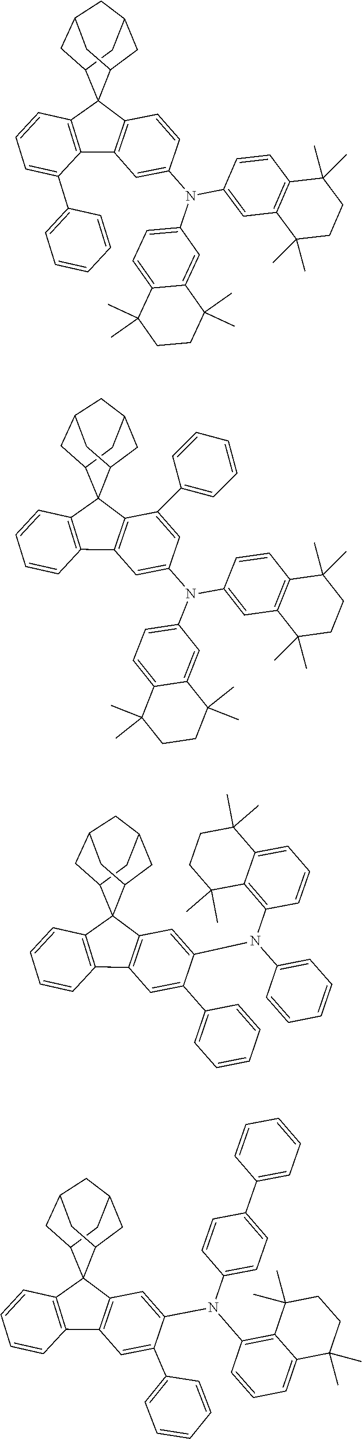 [SMILES: CC1(C)CCC(C)(C)c2cc(N(c3ccc4c(c3)-c3c(-c5ccccc5)cccc3C43C4CC5CC(C4)CC3C5)c3ccc4c(c3)C(C)(C)CCC4(C)C)ccc21 ; CC1(C)CCC(C)(C)c2cc(N(c3cc(-c4ccccc4)c4c(c3)-c3ccccc3C43C4CC5CC(C4)CC3C5)c3ccc4c(c3)C(C)(C)CCC4(C)C)ccc21 ; CC1(C)CCC(C)(C)c2c(N(c3ccccc3)c3cc4c(cc3-c3ccccc3)-c3ccccc3C43C4CC5CC(C4)CC3C5)cccc21 ; CC1(C)CCC(C)(C)c2c(N(c3ccc(-c4ccccc4)cc3)c3cc4c(cc3-c3ccccc3)-c3ccccc3C43C4CC5CC(C4)CC3C5)cccc21]

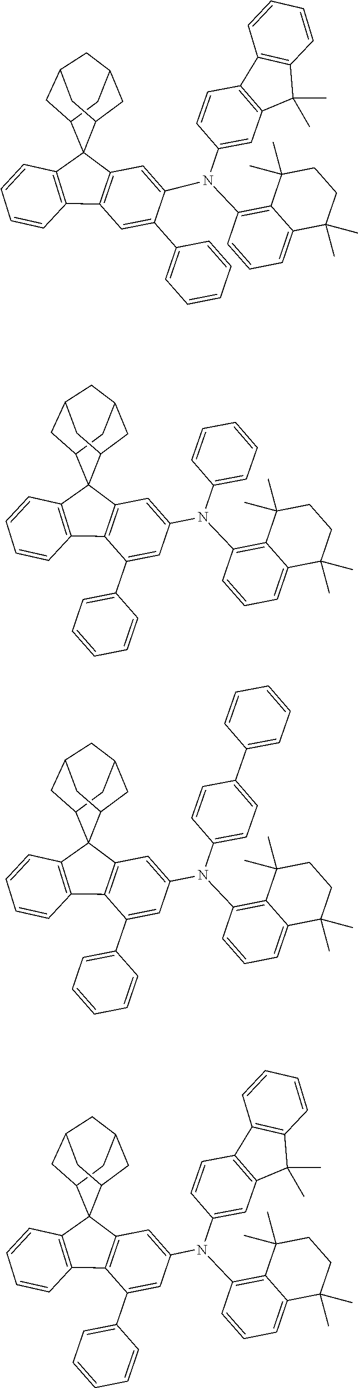 [SMILES: CC1(C)CCC(C)(C)c2c(N(c3ccc4c(c3)C(C)(C)c3ccccc3-4)c3cc4c(cc3-c3ccccc3)-c3ccccc3C43C4CC5CC(C4)CC3C5)cccc21 ; CC1(C)CCC(C)(C)c2c(N(c3ccccc3)c3cc(-c4ccccc4)c4c(c3)C3(c5ccccc5-4)C4CC5CC(C4)CC3C5)cccc21 ; CC1(C)CCC(C)(C)c2c(N(c3ccc(-c4ccccc4)cc3)c3cc(-c4ccccc4)c4c(c3)C3(c5ccccc5-4)C4CC5CC(C4)CC3C5)cccc21 ; CC1(C)CCC(C)(C)c2c(N(c3ccc4c(c3)C(C)(C)c3ccccc3-4)c3cc(-c4ccccc4)c4c(c3)C3(c5ccccc5-4)C4CC5CC(C4)CC3C5)cccc21]

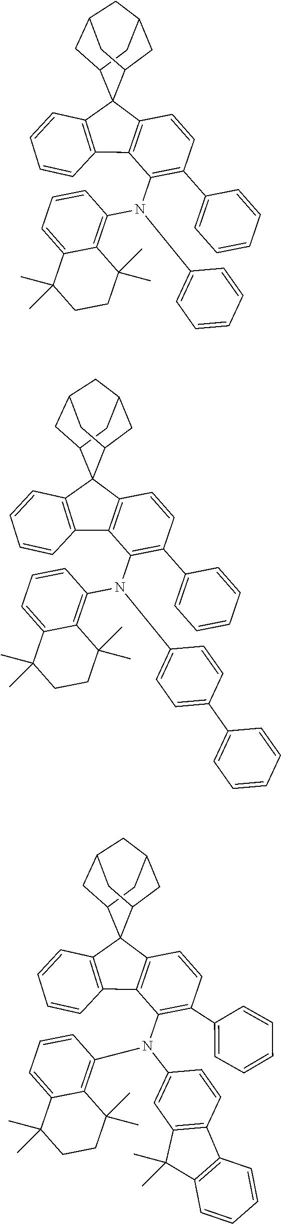 [SMILES: CC1(C)CCC(C)(C)c2c(N(c3ccccc3)c3c(-c4ccccc4)ccc4c3-c3ccccc3C43C4CC5CC(C4)CC3C5)cccc21 ; CC1(C)CCC(C)(C)c2c(N(c3ccc(-c4ccccc4)cc3)c3c(-c4ccccc4)ccc4c3-c3ccccc3C43C4CC5CC(C4)CC3C5)cccc21 ; CC1(C)CCC(C)(C)c2c(N(c3ccc4c(c3)C(C)(C)c3ccccc3-4)c3c(-c4ccccc4)ccc4c3-c3ccccc3C43C4CC5CC(C4)CC3C5)cccc21]

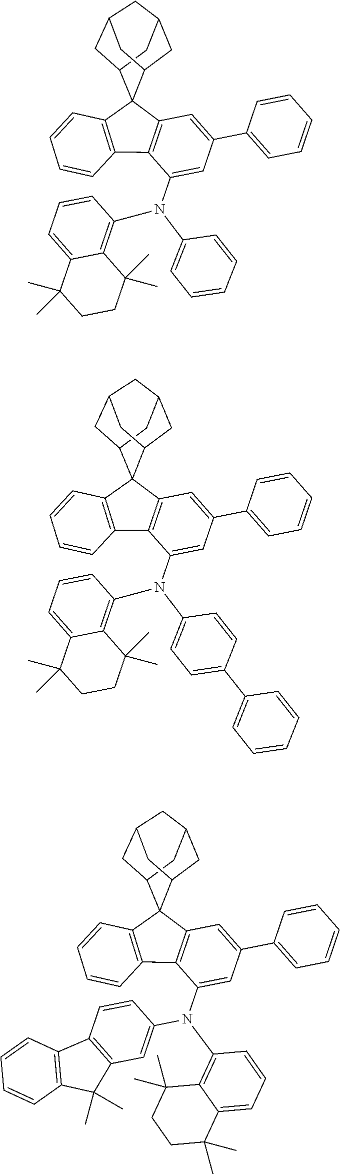 [SMILES: CC1(C)CCC(C)(C)c2c(N(c3ccccc3)c3cc(-c4ccccc4)cc4c3-c3ccccc3C43C4CC5CC(C4)CC3C5)cccc21 ; CC1(C)CCC(C)(C)c2c(N(c3ccc(-c4ccccc4)cc3)c3cc(-c4ccccc4)cc4c3-c3ccccc3C43C4CC5CC(C4)CC3C5)cccc21 ; CC1(C)CCC(C)(C)c2c(N(c3ccc4c(c3)C(C)(C)c3ccccc3-4)c3cc(-c4ccccc4)cc4c3-c3ccccc3C43C4CC5CC(C4)CC3C5)cccc21]

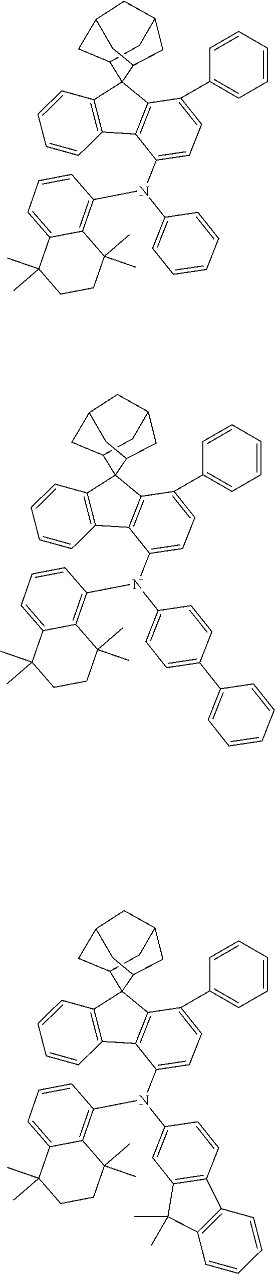 [SMILES: CC1(C)CCC(C)(C)c2c(N(c3ccccc3)c3ccc(-c4ccccc4)c4c3-c3ccccc3C43C4CC5CC(C4)CC3C5)cccc21 ; CC1(C)CCC(C)(C)c2c(N(c3ccc(-c4ccccc4)cc3)c3ccc(-c4ccccc4)c4c3-c3ccccc3C43C4CC5CC(C4)CC3C5)cccc21 ; CC1(C)CCC(C)(C)c2c(N(c3ccc4c(c3)C(C)(C)c3ccccc3-4)c3ccc(-c4ccccc4)c4c3-c3ccccc3C43C4CC5CC(C4)CC3C5)cccc21]

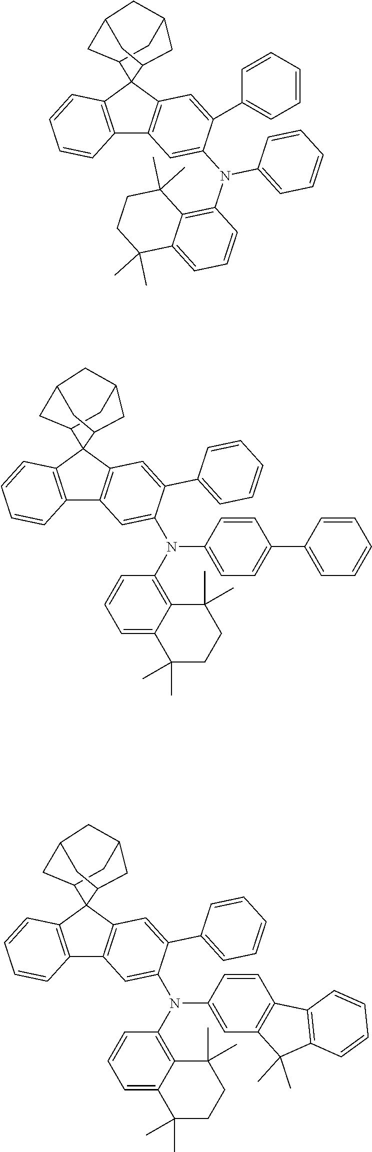 [SMILES: CC1(C)CCC(C)(C)c2c(N(c3ccccc3)c3cc4c(cc3-c3ccccc3)C3(c5ccccc5-4)C4CC5CC(C4)CC3C5)cccc21 ; CC1(C)CCC(C)(C)c2c(N(c3ccc(-c4ccccc4)cc3)c3cc4c(cc3-c3ccccc3)C3(c5ccccc5-4)C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)C3(c5ccccc5-4)C4CC5CC(C4)CC3C5)cccc21]

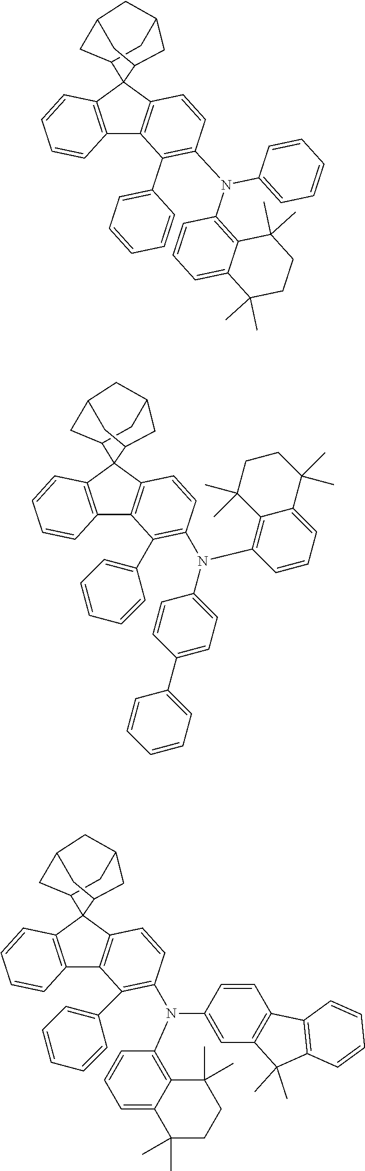 [SMILES: CC1(C)CCC(C)(C)c2c(N(c3ccccc3)c3ccc4c(c3-c3ccccc3)-c3ccccc3C43C4CC5CC(C4)CC3C5)cccc21 ; CC1(C)CCC(C)(C)c2c(N(c3ccc(-c4ccccc4)cc3)c3ccc4c(c3-c3ccccc3)-c3ccccc3C43C4CC5CC(C4)CC3C5)cccc21 ; CC1(C)CCC(C)(C)c2c(N(c3ccc4c(c3)C(C)(C)c3ccccc3-4)c3ccc4c(c3-c3ccccc3)-c3ccccc3C43C4CC5CC(C4)CC3C5)cccc21]

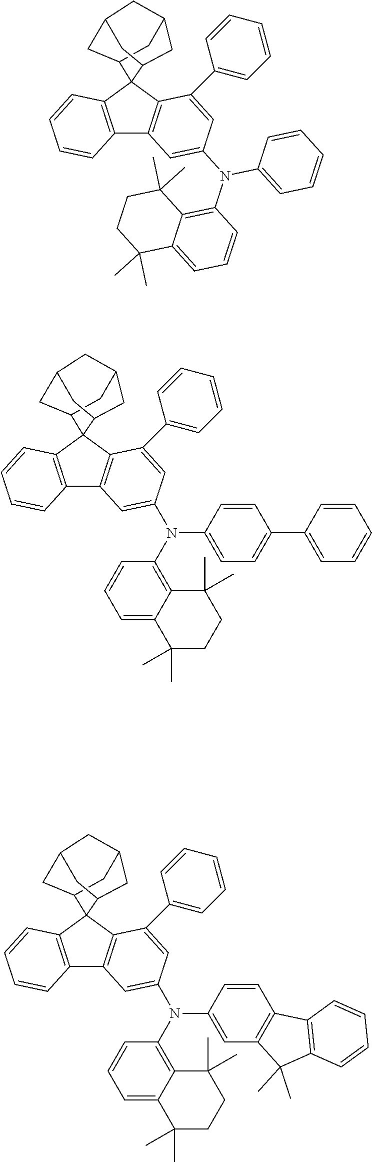 [SMILES: CC1(C)CCC(C)(C)c2c(N(c3ccccc3)c3cc(-c4ccccc4)c4c(c3)-c3ccccc3C43C4CC5CC(C4)CC3C5)cccc21 ; CC1(C)CCC(C)(C)c2c(N(c3ccc(-c4ccccc4)cc3)c3cc(-c4ccccc4)c4c(c3)-c3ccccc3C43C4CC5CC(C4)CC3C5)cccc21 ; CC1(C)CCC(C)(C)c2c(N(c3cc(-c4ccccc4)c4c(c3)-c3ccccc3C43C4CC5CC(C4)CC3C5)c3ccc4c(c3)C(C)(C)c3ccccc3-4)cccc21]

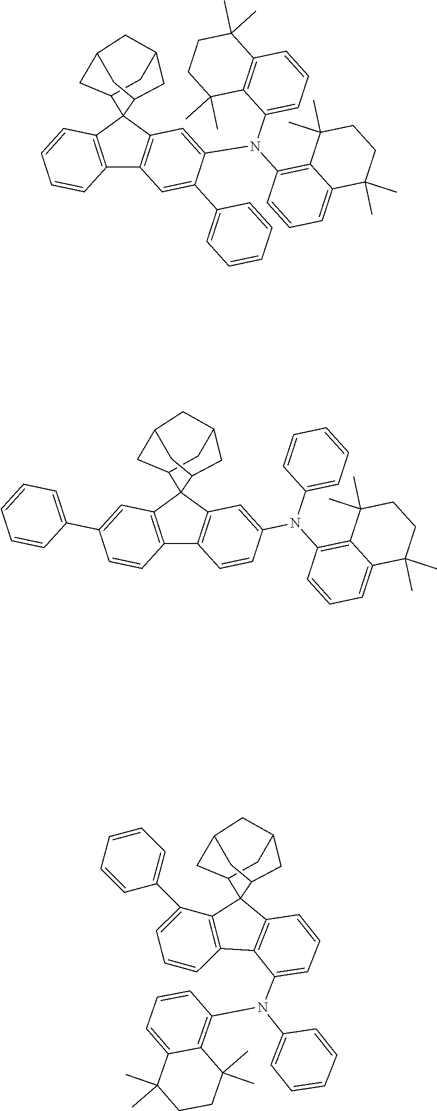 [SMILES: CC1(C)CCC(C)(C)c2c(N(c3cc4c(cc3-c3ccccc3)-c3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cc(-c6ccccc6)ccc5-4)C4CC5CC(C4)CC3C5)cccc21 ; CC1(C)CCC(C)(C)c2c(N(c3ccccc3)c3cccc4c3-c3cccc(-c5ccccc5)c3C43C4CC5CC(C4)CC3C5)cccc21]

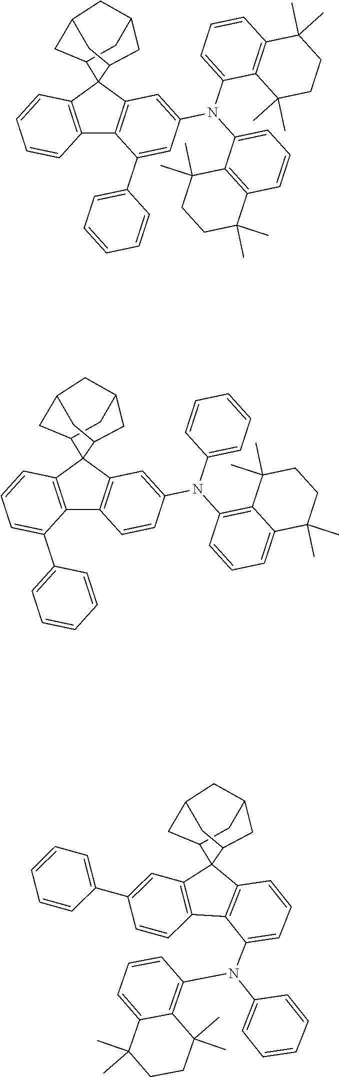 [SMILES: CC1(C)CCC(C)(C)c2c(N(c3cc(-c4ccccc4)c4c(c3)C3(c5ccccc5-4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cccc(-c6ccccc6)c5-4)C4CC5CC(C4)CC3C5)cccc21 ; CC1(C)CCC(C)(C)c2c(N(c3ccccc3)c3cccc4c3-c3ccc(-c5ccccc5)cc3C43C4CC5CC(C4)CC3C5)cccc21]

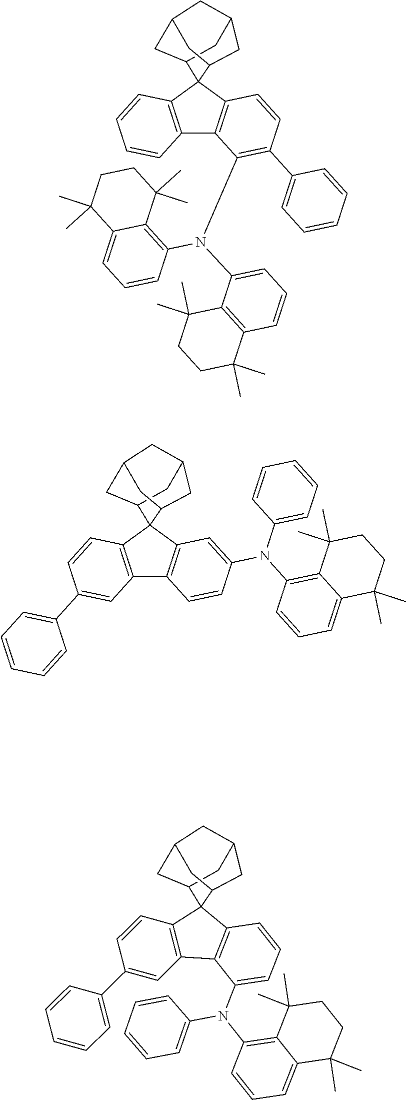 [SMILES: CC1(C)CCC(C)(C)c2c(N(c3cccc4c3C(C)(C)CCC4(C)C)c3c(-c4ccccc4)ccc4c3-c3ccccc3C43C4CC5CC(C4)CC3C5)cccc21 ; CC1(C)CCC(C)(C)c2c(N(c3ccccc3)c3ccc4c(c3)C3(c5ccc(-c6ccccc6)cc5-4)C4CC5CC(C4)CC3C5)cccc21 ; CC1(C)CCC(C)(C)c2c(N(c3ccccc3)c3cccc4c3-c3cc(-c5ccccc5)ccc3C43C4CC5CC(C4)CC3C5)cccc21]

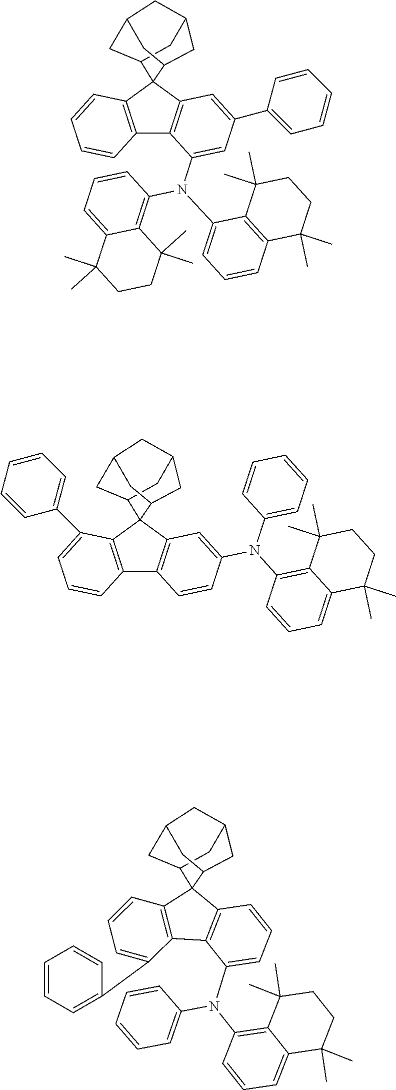

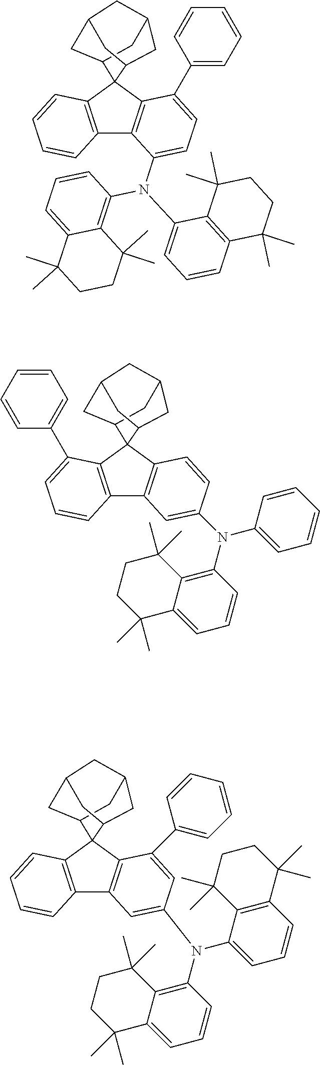

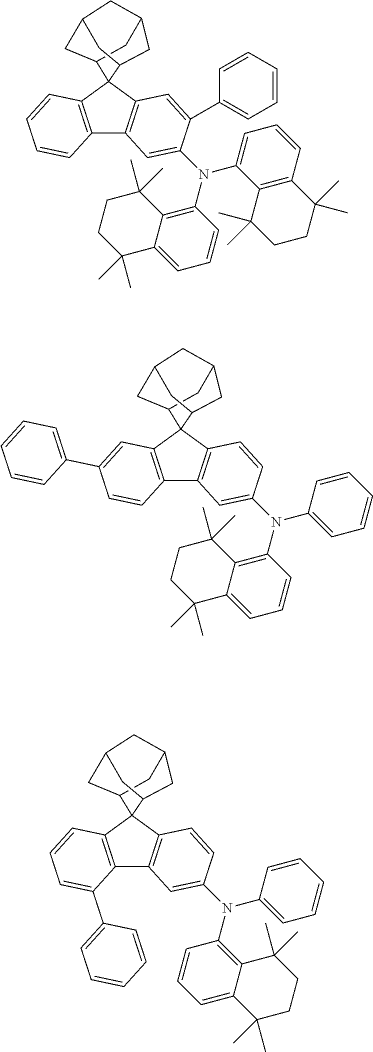 [SMILES: CC1(C)CCC(C)(C)c2c(N(c3cc4c(cc3-c3ccccc3)C3(c5ccccc5-4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)-c3ccc(-c5ccccc5)cc3C43C4CC5CC(C4)CC3C5)cccc21 ; CC1(C)CCC(C)(C)c2c(N(c3ccccc3)c3ccc4c(c3)-c3c(-c5ccccc5)cccc3C43C4CC5CC(C4)CC3C5)cccc21]

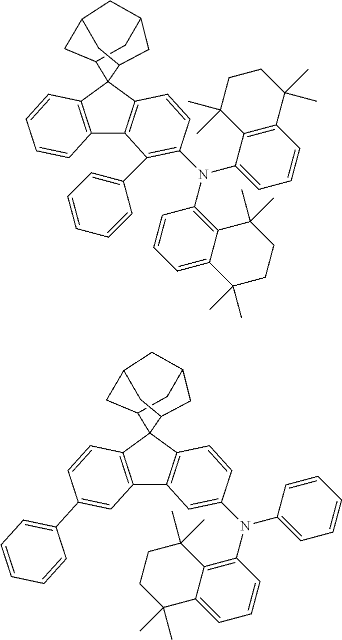 [SMILES: CC1(C)CCC(C)(C)c2c(N(c3ccc4c(c3-c3ccccc3)-c3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)-c3cc(-c5ccccc5)ccc3C43C4CC5CC(C4)CC3C5)cccc21]

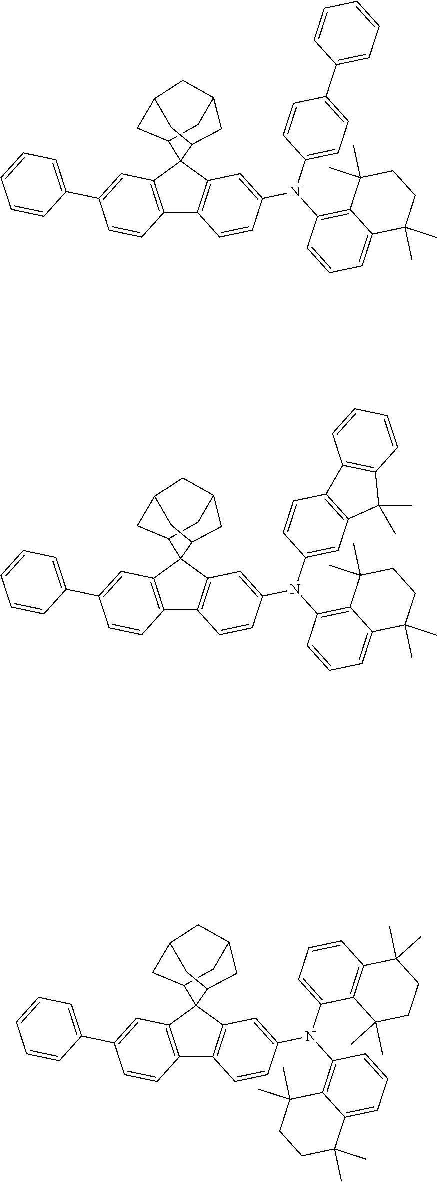 [SMILES: CC1(C)CCC(C)(C)c2c(N(c3ccc(-c4ccccc4)cc3)c3ccc4c(c3)C3(c5cc(-c6ccccc6)ccc5-4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cc(-c6ccccc6)ccc5-4)C4CC5CC(C4)CC3C5)cccc21 ; CC1(C)CCC(C)(C)c2c(N(c3ccc4c(c3)C3(c5cc(-c6ccccc6)ccc5-4)C4CC5CC(C4)CC3C5)c3cccc4c3C(C)(C)CCC4(C)C)cccc21]

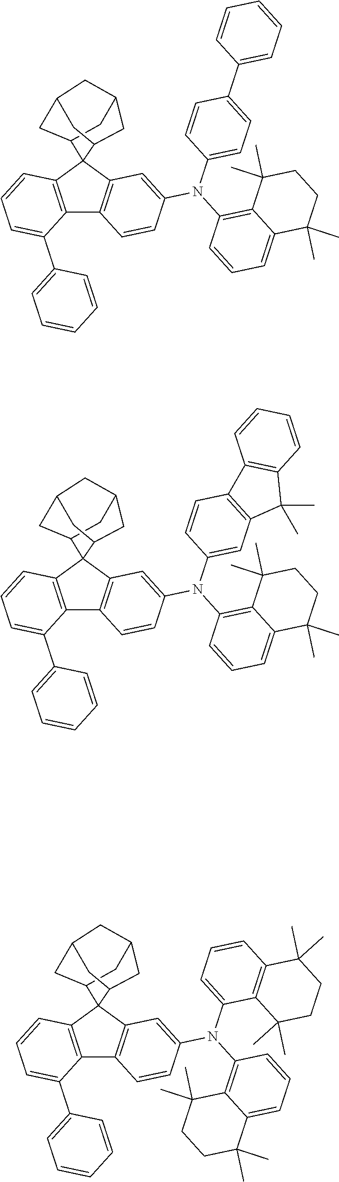 [SMILES: CC1(C)CCC(C)(C)c2c(N(c3ccc(-c4ccccc4)cc3)c3ccc4c(c3)C3(c5cccc(-c6ccccc6)c5-4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cccc(-c6ccccc6)c5-4)C4CC5CC(C4)CC3C5)cccc21 ; CC1(C)CCC(C)(C)c2c(N(c3ccc4c(c3)C3(c5cccc(-c6ccccc6)c5-4)C4CC5CC(C4)CC3C5)c3cccc4c3C(C)(C)CCC4(C)C)cccc21]

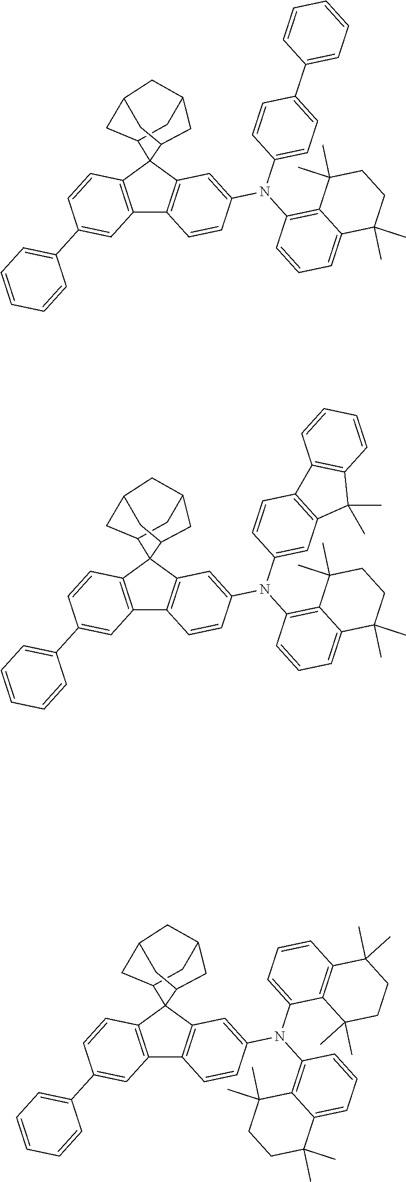 [SMILES: CC1(C)CCC(C)(C)c2c(N(c3ccc(-c4ccccc4)cc3)c3ccc4c(c3)C3(c5ccc(-c6ccccc6)cc5-4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5ccc(-c6ccccc6)cc5-4)C4CC5CC(C4)CC3C5)cccc21 ; CC1(C)CCC(C)(C)c2c(N(c3ccc4c(c3)C3(c5ccc(-c6ccccc6)cc5-4)C4CC5CC(C4)CC3C5)c3cccc4c3C(C)(C)CCC4(C)C)cccc21]

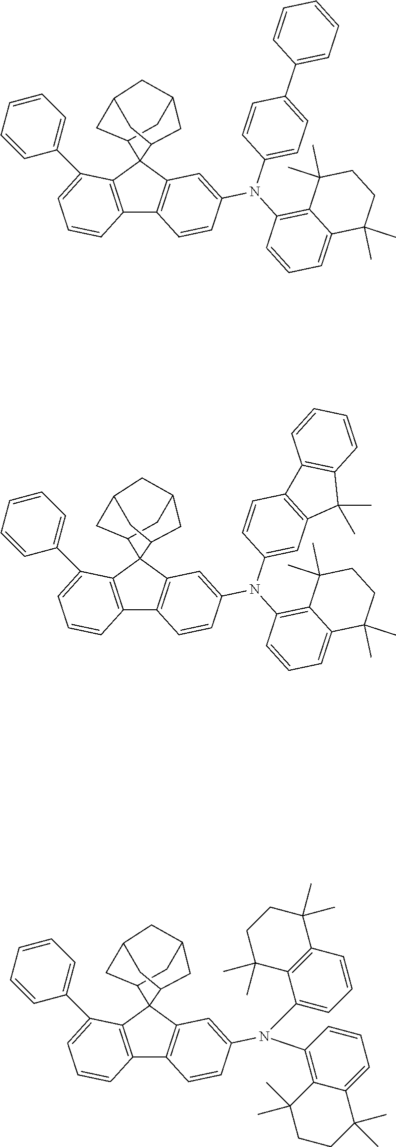 [SMILES: CC1(C)CCC(C)(C)c2c(N(c3ccc(-c4ccccc4)cc3)c3ccc4c(c3)C3(c5c(-c6ccccc6)cccc5-4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5c(-c6ccccc6)cccc5-4)C4CC5CC(C4)CC3C5)cccc21 ; CC1(C)CCC(C)(C)c2c(N(c3ccc4c(c3)C3(c5c(-c6ccccc6)cccc5-4)C4CC5CC(C4)CC3C5)c3cccc4c3C(C)(C)CCC4(C)C)cccc21]

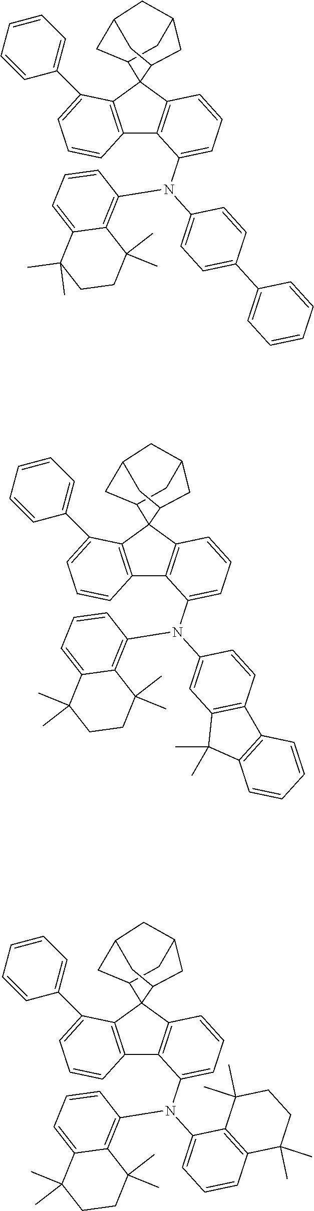 [SMILES: CC1(C)CCC(C)(C)c2c(N(c3ccc(-c4ccccc4)cc3)c3cccc4c3-c3cccc(-c5ccccc5)c3C43C4CC5CC(C4)CC3C5)cccc21 ; CC1(C)CCC(C)(C)c2c(N(c3ccc4c(c3)C(C)(C)c3ccccc3-4)c3cccc4c3-c3cccc(-c5ccccc5)c3C43C4CC5CC(C4)CC3C5)cccc21 ; CC1(C)CCC(C)(C)c2c(N(c3cccc4c3-c3cccc(-c5ccccc5)c3C43C4CC5CC(C4)CC3C5)c3cccc4c3C(C)(C)CCC4(C)C)cccc21]

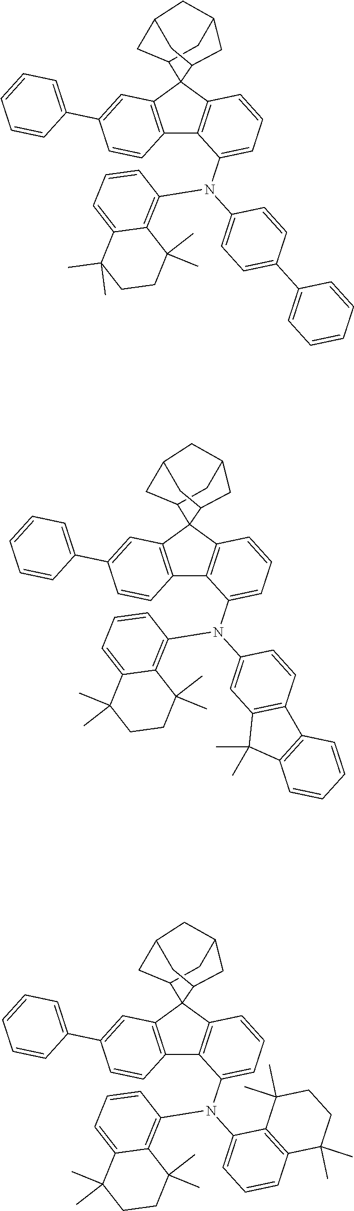 [SMILES: CC1(C)CCC(C)(C)c2c(N(c3ccc(-c4ccccc4)cc3)c3cccc4c3-c3ccc(-c5ccccc5)cc3C43C4CC5CC(C4)CC3C5)cccc21 ; CC1(C)CCC(C)(C)c2c(N(c3ccc4c(c3)C(C)(C)c3ccccc3-4)c3cccc4c3-c3ccc(-c5ccccc5)cc3C43C4CC5CC(C4)CC3C5)cccc21 ; CC1(C)CCC(C)(C)c2c(N(c3cccc4c3-c3ccc(-c5ccccc5)cc3C43C4CC5CC(C4)CC3C5)c3cccc4c3C(C)(C)CCC4(C)C)cccc21]

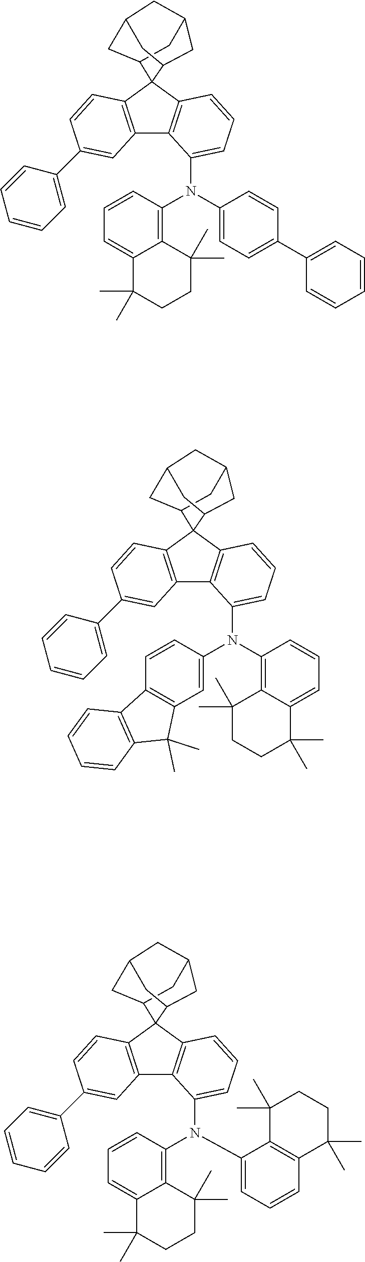 [SMILES: CC1(C)CCC(C)(C)c2c(N(c3ccc(-c4ccccc4)cc3)c3cccc4c3-c3cc(-c5ccccc5)ccc3C43C4CC5CC(C4)CC3C5)cccc21 ; CC1(C)CCC(C)(C)c2c(N(c3ccc4c(c3)C(C)(C)c3ccccc3-4)c3cccc4c3-c3cc(-c5ccccc5)ccc3C43C4CC5CC(C4)CC3C5)cccc21 ; CC1(C)CCC(C)(C)c2c(N(c3cccc4c3-c3cc(-c5ccccc5)ccc3C43C4CC5CC(C4)CC3C5)c3cccc4c3C(C)(C)CCC4(C)C)cccc21]

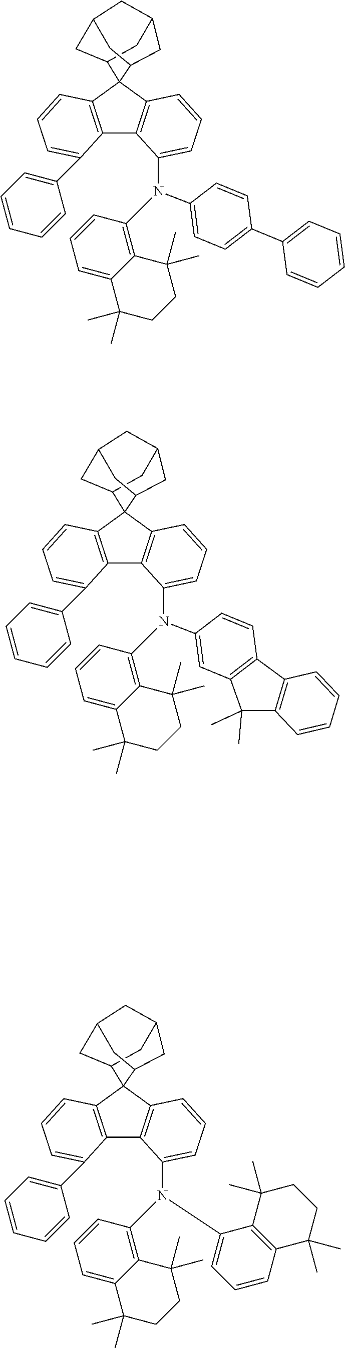 [SMILES: CC1(C)CCC(C)(C)c2c(N(c3ccc(-c4ccccc4)cc3)c3cccc4c3-c3c(-c5ccccc5)cccc3C43C4CC5CC(C4)CC3C5)cccc21 ; CC1(C)CCC(C)(C)c2c(N(c3ccc4c(c3)C(C)(C)c3ccccc3-4)c3cccc4c3-c3c(-c5ccccc5)cccc3C43C4CC5CC(C4)CC3C5)cccc21 ; CC1(C)CCC(C)(C)c2c(N(c3cccc4c3-c3c(-c5ccccc5)cccc3C43C4CC5CC(C4)CC3C5)c3cccc4c3C(C)(C)CCC4(C)C)cccc21]

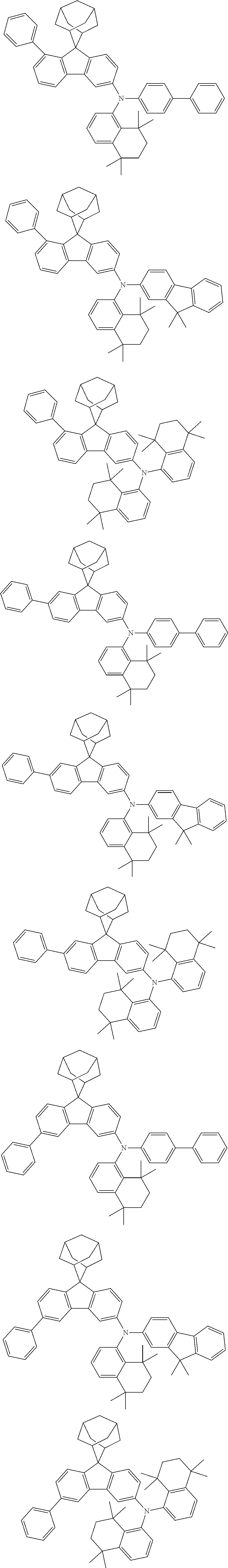

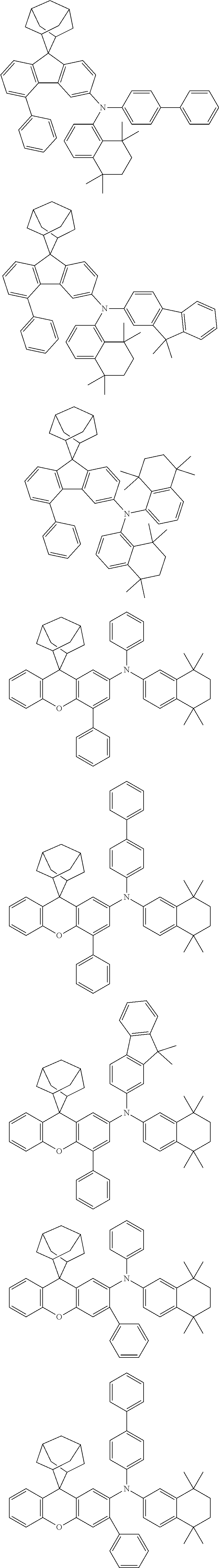

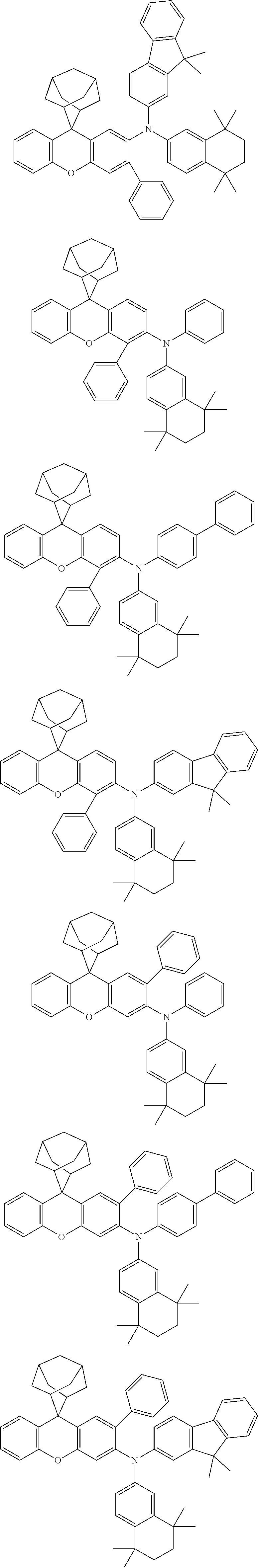

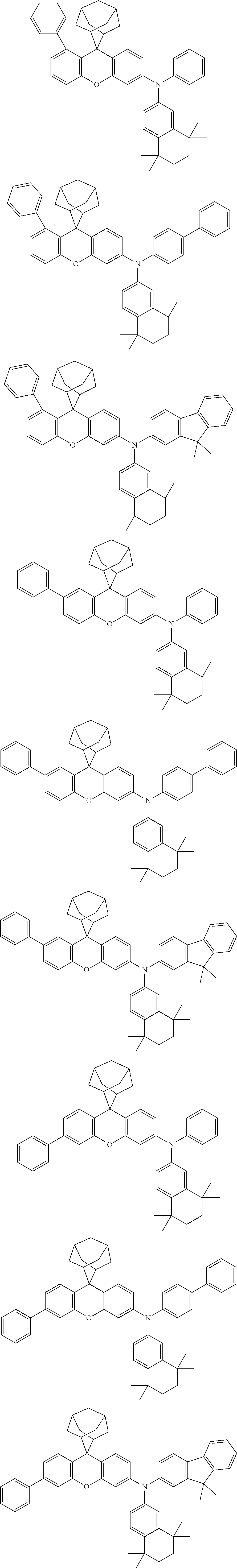

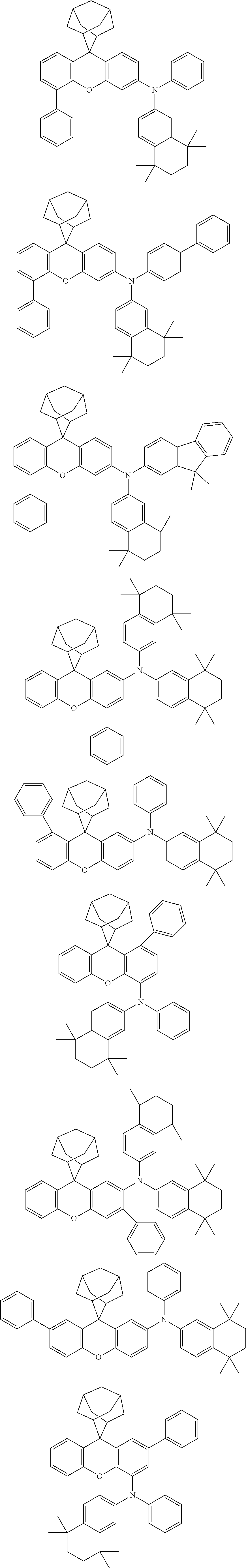

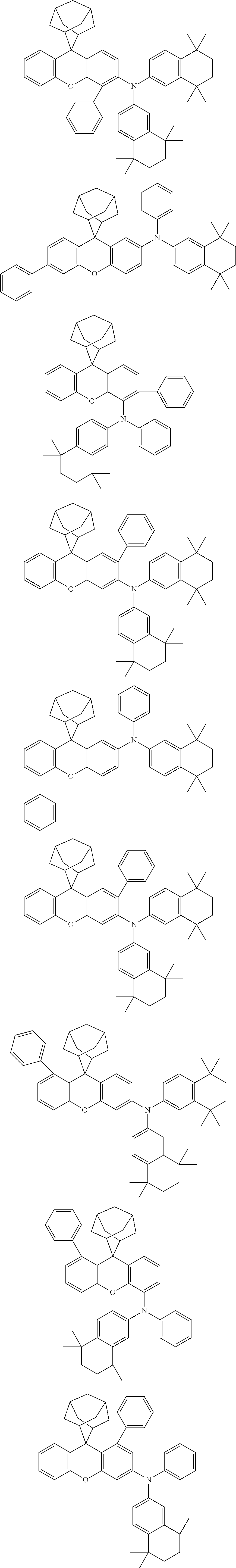

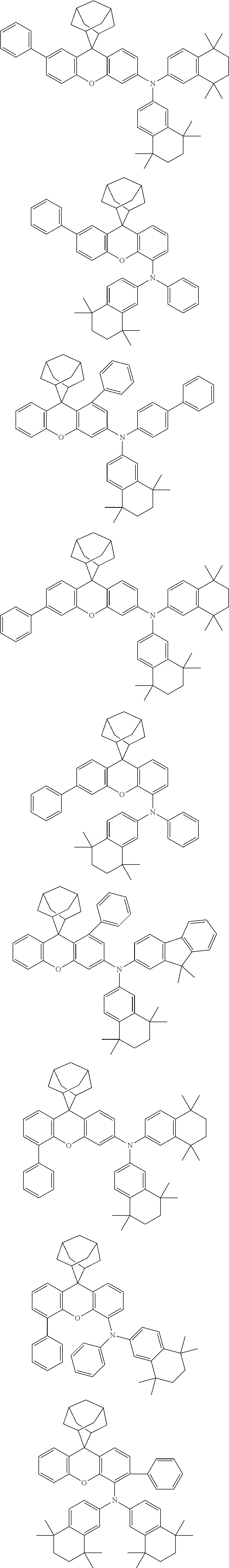

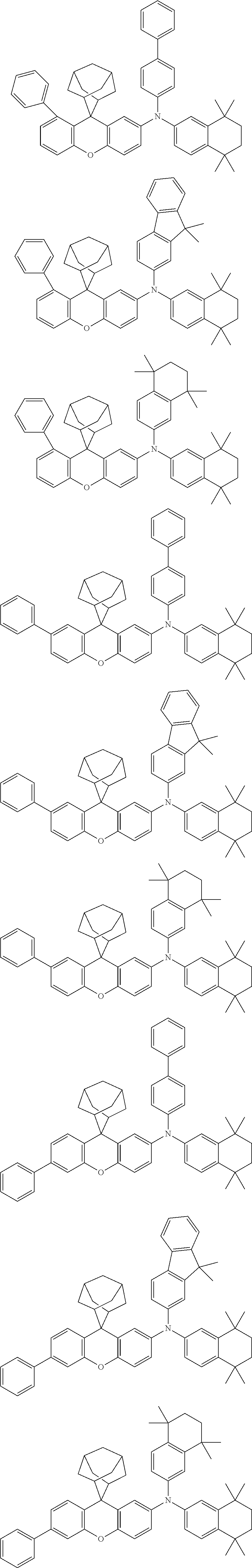

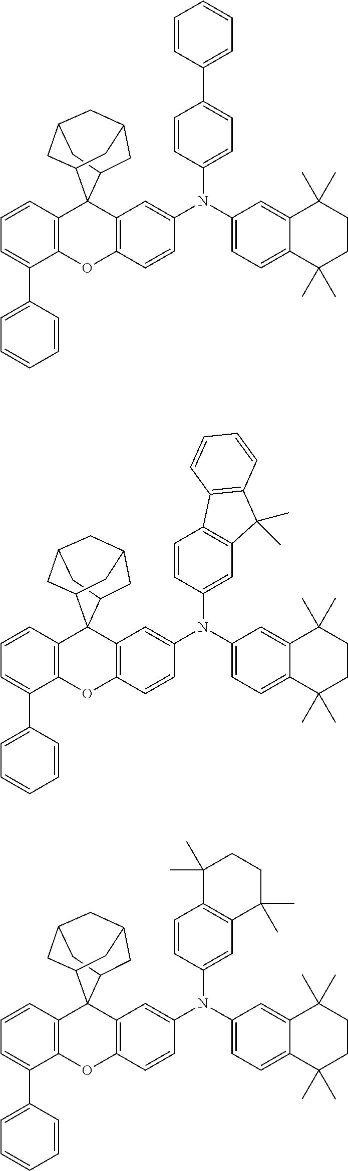 [SMILES: CC1(C)CCC(C)(C)c2cc(N(c3ccc(-c4ccccc4)cc3)c3ccc4c(c3)C3(c5cccc(-c6ccccc6)c5O4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5cccc(-c6ccccc6)c5O4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5cccc(-c6ccccc6)c5O4)C4CC5CC(C4)CC3C5)ccc21]

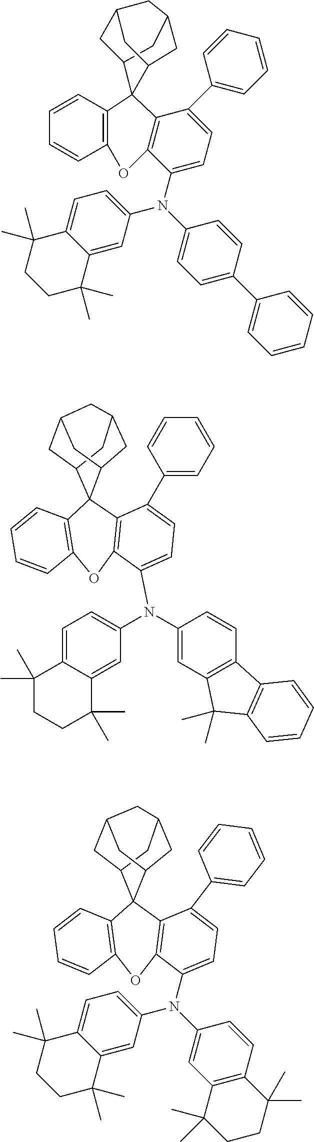

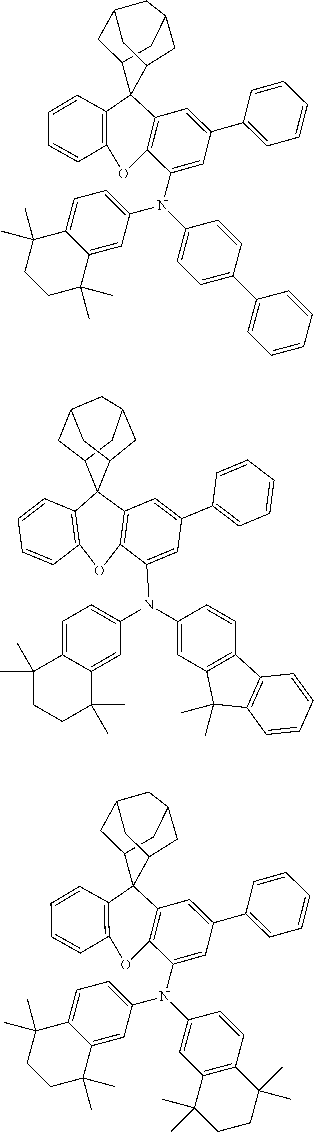 [SMILES: CC1(C)CCC(C)(C)c2cc(N(c3ccc(-c4ccccc4)cc3)c3cc(-c4ccccc4)cc4c3Oc3ccccc3C43C4CC5CC(C4)CC3C5)ccc21 ; CC1(C)CCC(C)(C)c2cc(N(c3ccc4c(c3)C(C)(C)c3ccccc3-4)c3cc(-c4ccccc4)cc4c3Oc3ccccc3C43C4CC5CC(C4)CC3C5)ccc21 ; CC1(C)CCC(C)(C)c2cc(N(c3ccc4c(c3)C(C)(C)CCC4(C)C)c3cc(-c4ccccc4)cc4c3Oc3ccccc3C43C4CC5CC(C4)CC3C5)ccc21]

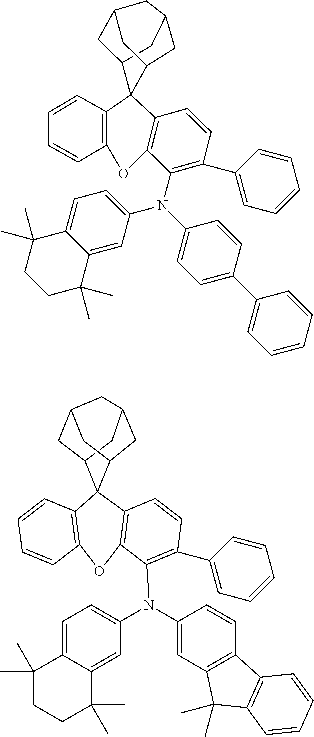 [SMILES: CC1(C)CCC(C)(C)c2cc(N(c3ccc(-c4ccccc4)cc3)c3c(-c4ccccc4)ccc4c3Oc3ccccc3C43C4CC5CC(C4)CC3C5)ccc21 ; CC1(C)CCC(C)(C)c2cc(N(c3ccc4c(c3)C(C)(C)c3ccccc3-4)c3c(-c4ccccc4)ccc4c3Oc3ccccc3C43C4CC5CC(C4)CC3C5)ccc21]

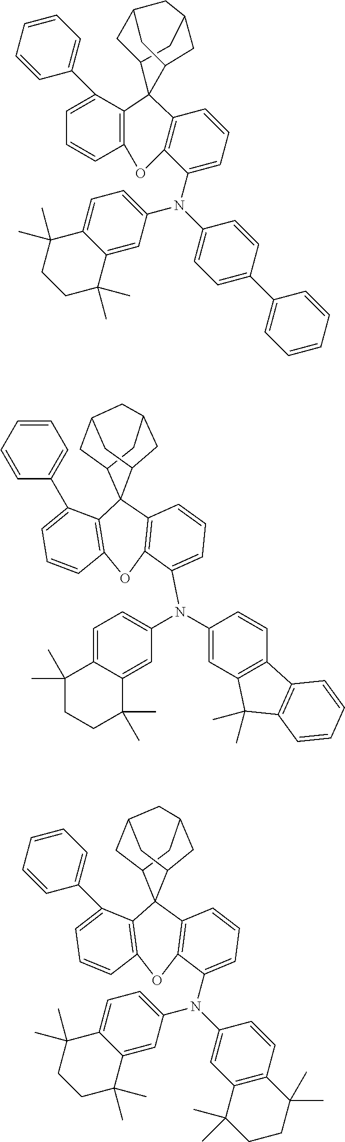 [SMILES: CC1(C)CCC(C)(C)c2cc(N(c3ccc(-c4ccccc4)cc3)c3cccc4c3Oc3cccc(-c5ccccc5)c3C43C4CC5CC(C4)CC3C5)ccc21 ; CC1(C)CCC(C)(C)c2cc(N(c3ccc4c(c3)C(C)(C)c3ccccc3-4)c3cccc4c3Oc3cccc(-c5ccccc5)c3C43C4CC5CC(C4)CC3C5)ccc21 ; CC1(C)CCC(C)(C)c2cc(N(c3ccc4c(c3)C(C)(C)CCC4(C)C)c3cccc4c3Oc3cccc(-c5ccccc5)c3C43C4CC5CC(C4)CC3C5)ccc21]

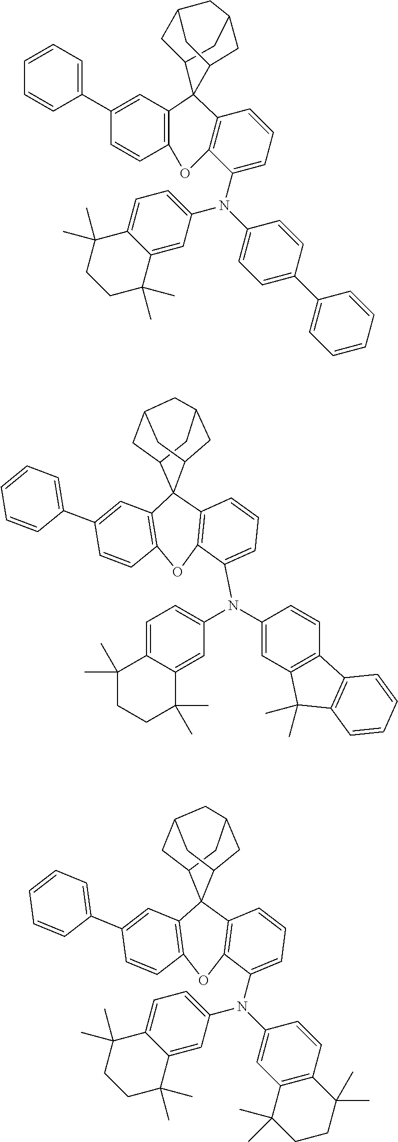 [SMILES: CC1(C)CCC(C)(C)c2cc(N(c3ccc(-c4ccccc4)cc3)c3cccc4c3Oc3ccc(-c5ccccc5)cc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3ccc(-c5ccccc5)cc3C43C4CC5CC(C4)CC3C5)ccc21 ; CC1(C)CCC(C)(C)c2cc(N(c3ccc4c(c3)C(C)(C)CCC4(C)C)c3cccc4c3Oc3ccc(-c5ccccc5)cc3C43C4CC5CC(C4)CC3C5)ccc21]

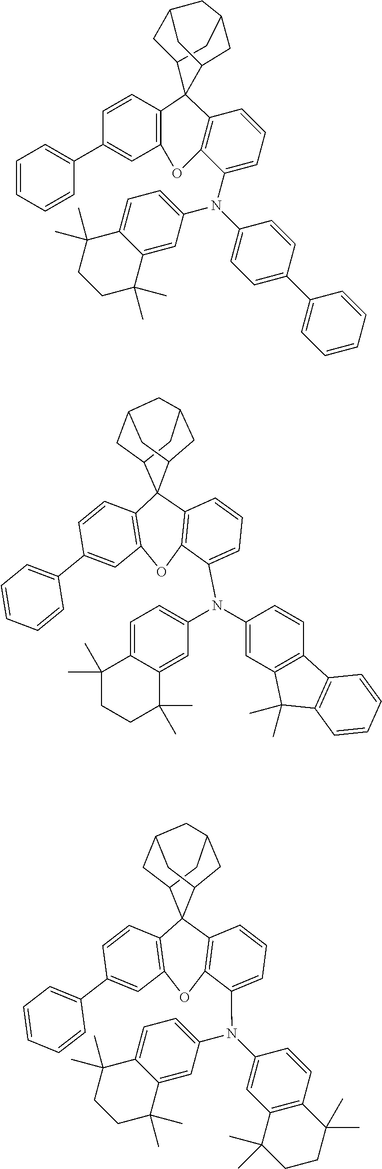 [SMILES: CC1(C)CCC(C)(C)c2cc(N(c3ccc(-c4ccccc4)cc3)c3cccc4c3Oc3cc(-c5ccccc5)ccc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3cc(-c5ccccc5)ccc3C43C4CC5CC(C4)CC3C5)ccc21 ; CC1(C)CCC(C)(C)c2cc(N(c3ccc4c(c3)C(C)(C)CCC4(C)C)c3cccc4c3Oc3cc(-c5ccccc5)ccc3C43C4CC5CC(C4)CC3C5)ccc21]

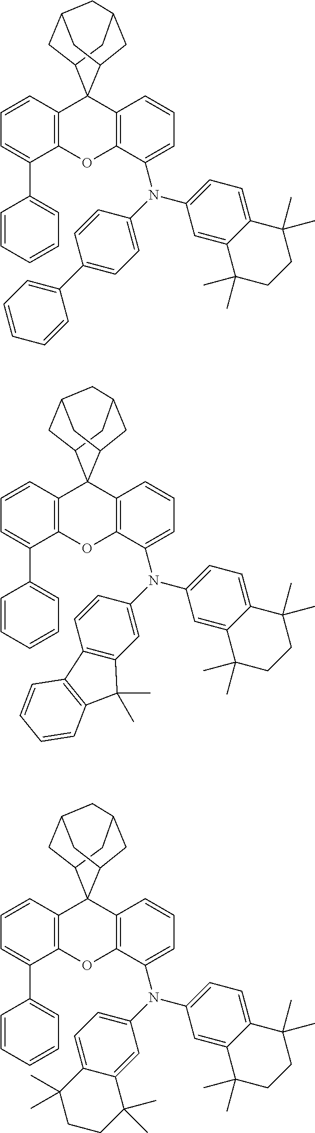 [SMILES: CC1(C)CCC(C)(C)c2cc(N(c3ccc(-c4ccccc4)cc3)c3cccc4c3Oc3c(-c5ccccc5)cccc3C43C4CC5CC(C4)CC3C5)ccc21 ; CC1(C)CCC(C)(C)c2cc(N(c3ccc4c(c3)C(C)(C)c3ccccc3-4)c3cccc4c3Oc3c(-c5ccccc5)cccc3C43C4CC5CC(C4)CC3C5)ccc21 ; CC1(C)CCC(C)(C)c2cc(N(c3ccc4c(c3)C(C)(C)CCC4(C)C)c3cccc4c3Oc3c(-c5ccccc5)cccc3C43C4CC5CC(C4)CC3C5)ccc21]

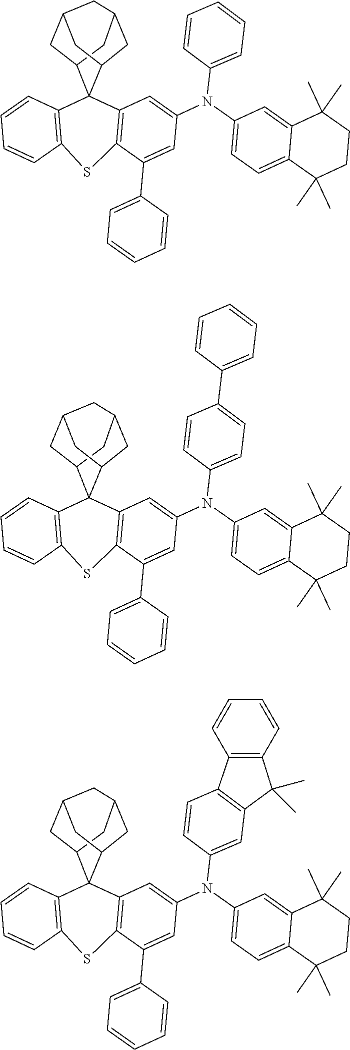 [SMILES: CC1(C)CCC(C)(C)c2cc(N(c3ccccc3)c3cc(-c4ccccc4)c4c(c3)C3(c5ccccc5S4)C4CC5CC(C4)CC3C5)ccc21 ; CC1(C)CCC(C)(C)c2cc(N(c3ccc(-c4ccccc4)cc3)c3cc(-c4ccccc4)c4c(c3)C3(c5ccccc5S4)C4CC5CC(C4)CC3C5)ccc21 ; CC1(C)CCC(C)(C)c2cc(N(c3ccc4c(c3)C(C)(C)c3ccccc3-4)c3cc(-c4ccccc4)c4c(c3)C3(c5ccccc5S4)C4CC5CC(C4)CC3C5)ccc21]

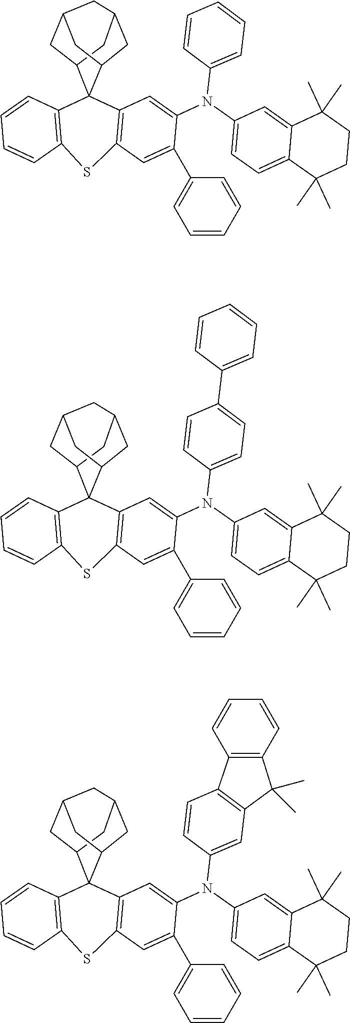 [SMILES: CC1(C)CCC(C)(C)c2cc(N(c3ccccc3)c3cc4c(cc3-c3ccccc3)Sc3ccccc3C43C4CC5CC(C4)CC3C5)ccc21 ; CC1(C)CCC(C)(C)c2cc(N(c3ccc(-c4ccccc4)cc3)c3cc4c(cc3-c3ccccc3)Sc3ccccc3C43C4CC5CC(C4)CC3C5)ccc21 ; CC1(C)CCC(C)(C)c2cc(N(c3ccc4c(c3)C(C)(C)c3ccccc3-4)c3cc4c(cc3-c3ccccc3)Sc3ccccc3C43C4CC5CC(C4)CC3C5)ccc21]

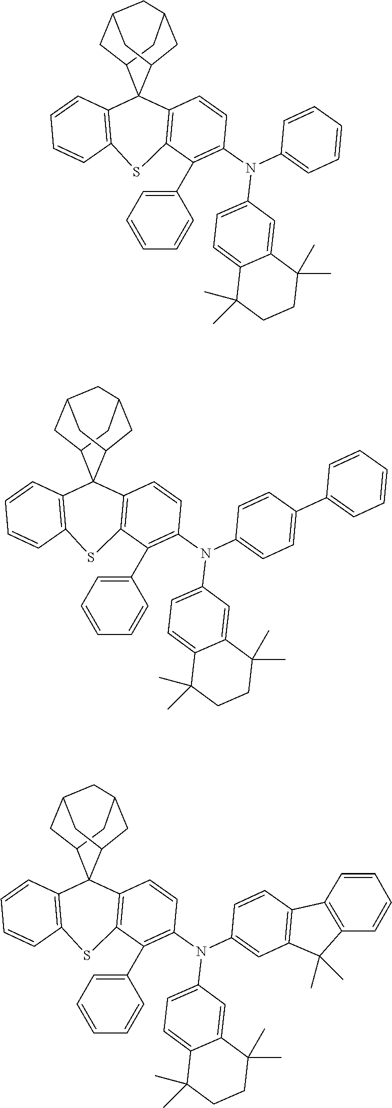 [SMILES: CC1(C)CCC(C)(C)c2cc(N(c3ccccc3)c3ccc4c(c3-c3ccccc3)Sc3ccccc3C43C4CC5CC(C4)CC3C5)ccc21 ; CC1(C)CCC(C)(C)c2cc(N(c3ccc(-c4ccccc4)cc3)c3ccc4c(c3-c3ccccc3)Sc3ccccc3C43C4CC5CC(C4)CC3C5)ccc21 ; CC1(C)CCC(C)(C)c2cc(N(c3ccc4c(c3)C(C)(C)c3ccccc3-4)c3ccc4c(c3-c3ccccc3)Sc3ccccc3C43C4CC5CC(C4)CC3C5)ccc21]

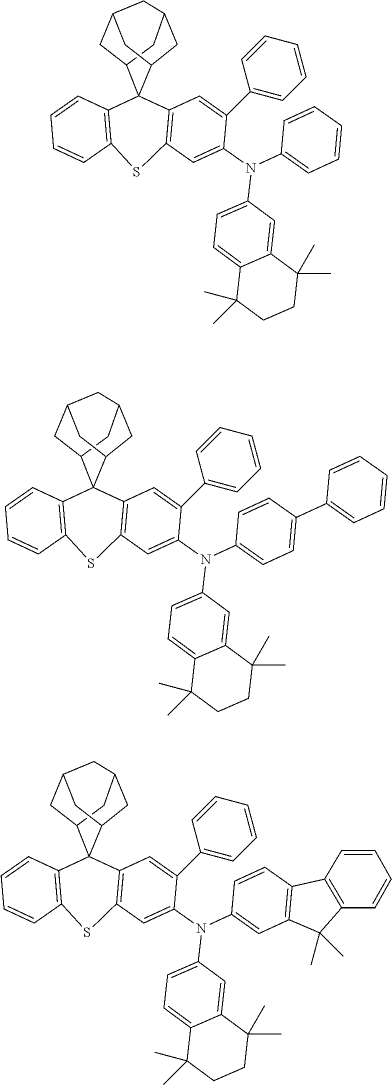 [SMILES: CC1(C)CCC(C)(C)c2cc(N(c3ccccc3)c3cc4c(cc3-c3ccccc3)C3(c5ccccc5S4)C4CC5CC(C4)CC3C5)ccc21 ; CC1(C)CCC(C)(C)c2cc(N(c3ccc(-c4ccccc4)cc3)c3cc4c(cc3-c3ccccc3)C3(c5ccccc5S4)C4CC5CC(C4)CC3C5)ccc21 ; CC1(C)CCC(C)(C)c2cc(N(c3ccc4c(c3)C(C)(C)c3ccccc3-4)c3cc4c(cc3-c3ccccc3)C3(c5ccccc5S4)C4CC5CC(C4)CC3C5)ccc21]

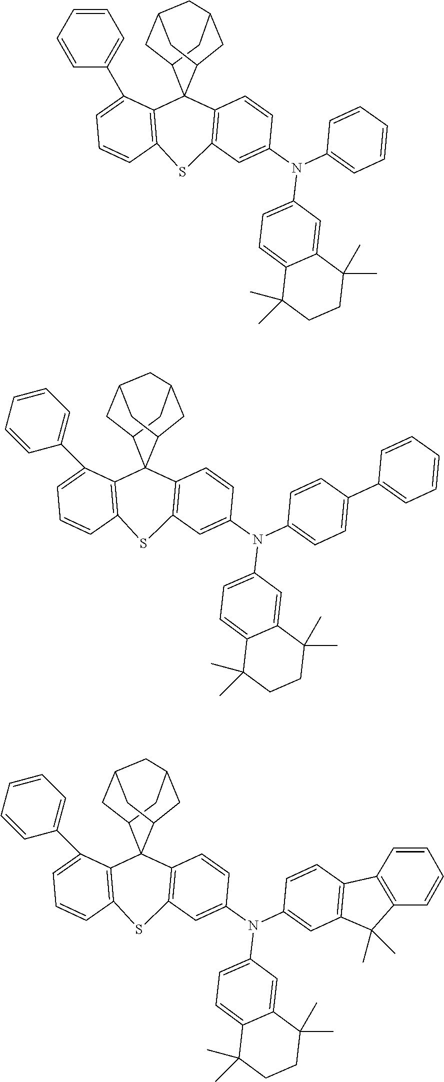 [SMILES: CC1(C)CCC(C)(C)c2cc(N(c3ccccc3)c3ccc4c(c3)Sc3cccc(-c5ccccc5)c3C43C4CC5CC(C4)CC3C5)ccc21 ; CC1(C)CCC(C)(C)c2cc(N(c3ccc(-c4ccccc4)cc3)c3ccc4c(c3)Sc3cccc(-c5ccccc5)c3C43C4CC5CC(C4)CC3C5)ccc21 ; CC1(C)CCC(C)(C)c2cc(N(c3ccc4c(c3)Sc3cccc(-c5ccccc5)c3C43C4CC5CC(C4)CC3C5)c3ccc4c(c3)C(C)(C)c3ccccc3-4)ccc21]

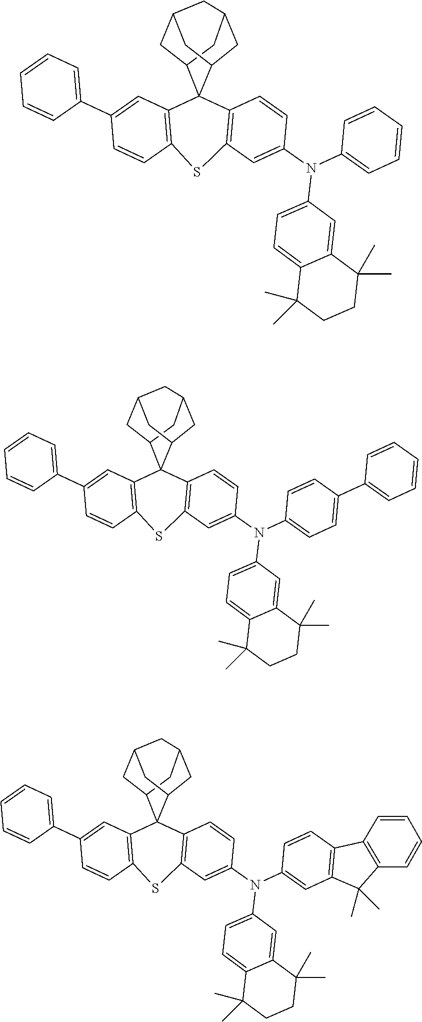 [SMILES: CC1(C)CCC(C)(C)c2cc(N(c3ccccc3)c3ccc4c(c3)Sc3ccc(-c5ccccc5)cc3C43C4CC5CC(C4)CC3C5)ccc21 ; CC1(C)CCC(C)(C)c2cc(N(c3ccc(-c4ccccc4)cc3)c3ccc4c(c3)Sc3ccc(-c5ccccc5)cc3C43C4CC5CC(C4)CC3C5)ccc21 ; CC1(C)CCC(C)(C)c2cc(N(c3ccc4c(c3)Sc3ccc(-c5ccccc5)cc3C43C4CC5CC(C4)CC3C5)c3ccc4c(c3)C(C)(C)c3ccccc3-4)ccc21]

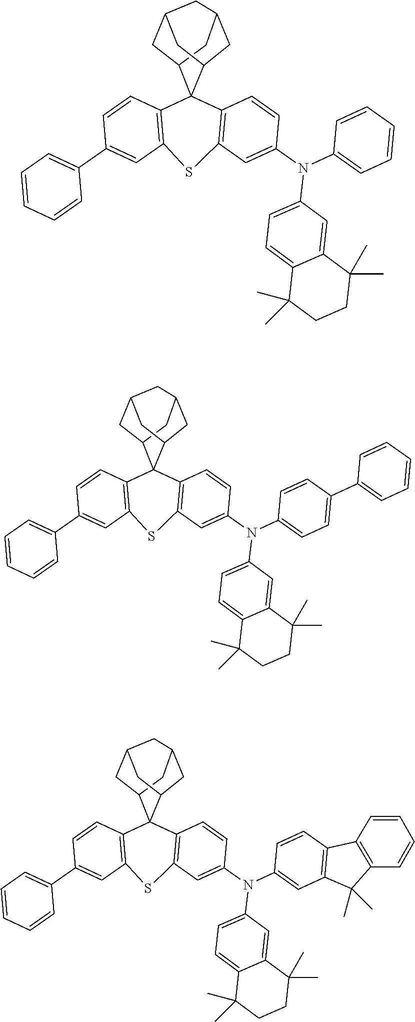 [SMILES: CC1(C)CCC(C)(C)c2cc(N(c3ccccc3)c3ccc4c(c3)Sc3cc(-c5ccccc5)ccc3C43C4CC5CC(C4)CC3C5)ccc21 ; CC1(C)CCC(C)(C)c2cc(N(c3ccc(-c4ccccc4)cc3)c3ccc4c(c3)Sc3cc(-c5ccccc5)ccc3C43C4CC5CC(C4)CC3C5)ccc21 ; CC1(C)CCC(C)(C)c2cc(N(c3ccc4c(c3)Sc3cc(-c5ccccc5)ccc3C43C4CC5CC(C4)CC3C5)c3ccc4c(c3)C(C)(C)c3ccccc3-4)ccc21]

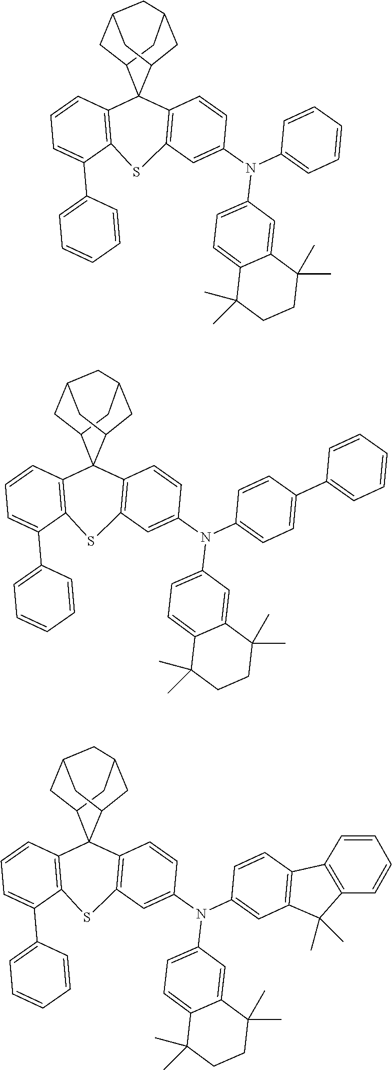 [SMILES: CC1(C)CCC(C)(C)c2cc(N(c3ccccc3)c3ccc4c(c3)Sc3c(-c5ccccc5)cccc3C43C4CC5CC(C4)CC3C5)ccc21 ; CC1(C)CCC(C)(C)c2cc(N(c3ccc(-c4ccccc4)cc3)c3ccc4c(c3)Sc3c(-c5ccccc5)cccc3C43C4CC5CC(C4)CC3C5)ccc21 ; CC1(C)CCC(C)(C)c2cc(N(c3ccc4c(c3)Sc3c(-c5ccccc5)cccc3C43C4CC5CC(C4)CC3C5)c3ccc4c(c3)C(C)(C)c3ccccc3-4)ccc21]

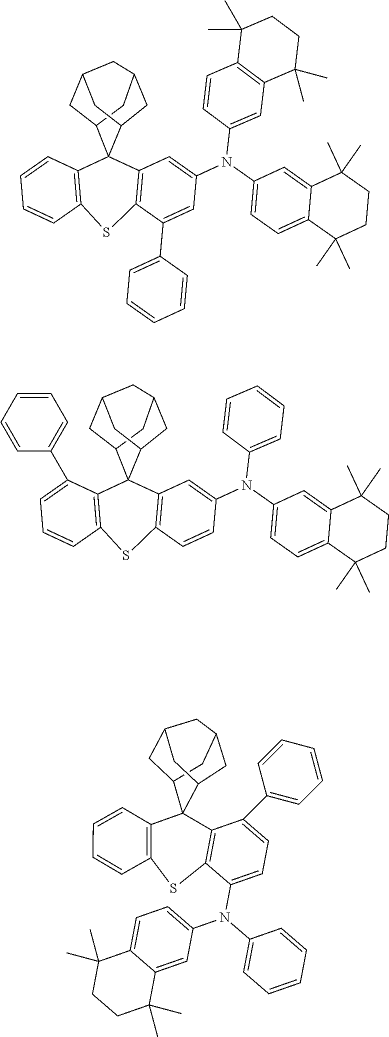 [SMILES: CC1(C)CCC(C)(C)c2cc(N(c3ccc4c(c3)C(C)(C)CCC4(C)C)c3cc(-c4ccccc4)c4c(c3)C3(c5ccccc5S4)C4CC5CC(C4)CC3C5)ccc21 ; CC1(C)CCC(C)(C)c2cc(N(c3ccccc3)c3ccc4c(c3)C3(c5c(cccc5-c5ccccc5)S4)C4CC5CC(C4)CC3C5)ccc21 ; CC1(C)CCC(C)(C)c2cc(N(c3ccccc3)c3ccc(-c4ccccc4)c4c3Sc3ccccc3C43C4CC5CC(C4)CC3C5)ccc21]

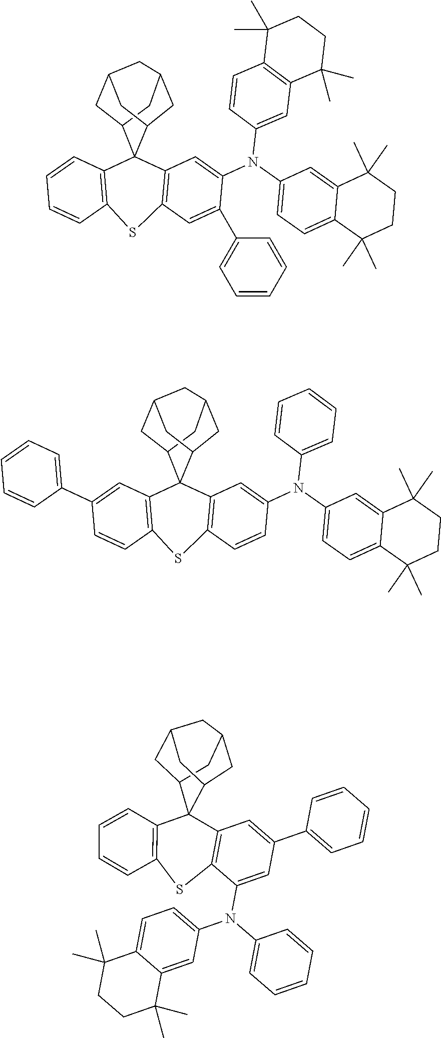 [SMILES: CC1(C)CCC(C)(C)c2cc(N(c3ccc4c(c3)C(C)(C)CCC4(C)C)c3cc4c(cc3-c3ccccc3)Sc3ccccc3C43C4CC5CC(C4)CC3C5)ccc21 ; CC1(C)CCC(C)(C)c2cc(N(c3ccccc3)c3ccc4c(c3)C3(c5cc(-c6ccccc6)ccc5S4)C4CC5CC(C4)CC3C5)ccc21 ; CC1(C)CCC(C)(C)c2cc(N(c3ccccc3)c3cc(-c4ccccc4)cc4c3Sc3ccccc3C43C4CC5CC(C4)CC3C5)ccc21]

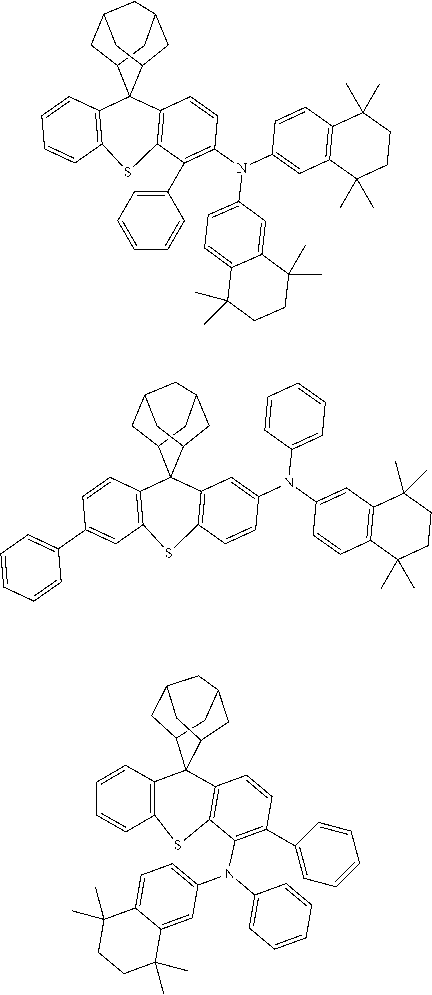 [SMILES: CC1(C)CCC(C)(C)c2cc(N(c3ccc4c(c3)C(C)(C)CCC4(C)C)c3ccc4c(c3-c3ccccc3)Sc3ccccc3C43C4CC5CC(C4)CC3C5)ccc21 ; CC1(C)CCC(C)(C)c2cc(N(c3ccccc3)c3ccc4c(c3)C3(c5ccc(-c6ccccc6)cc5S4)C4CC5CC(C4)CC3C5)ccc21 ; CC1(C)CCC(C)(C)c2cc(N(c3ccccc3)c3c(-c4ccccc4)ccc4c3Sc3ccccc3C43C4CC5CC(C4)CC3C5)ccc21]

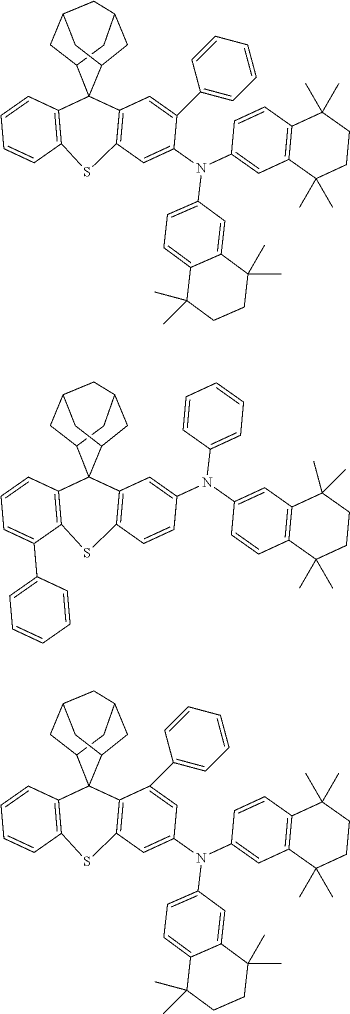 [SMILES: CC1(C)CCC(C)(C)c2cc(N(c3ccc4c(c3)C(C)(C)CCC4(C)C)c3cc4c(cc3-c3ccccc3)C3(c5ccccc5S4)C4CC5CC(C4)CC3C5)ccc21 ; CC1(C)CCC(C)(C)c2cc(N(c3ccccc3)c3ccc4c(c3)C3(c5cccc(-c6ccccc6)c5S4)C4CC5CC(C4)CC3C5)ccc21 ; CC1(C)CCC(C)(C)c2cc(N(c3cc4c(c(-c5ccccc5)c3)C3(c5ccccc5S4)C4CC5CC(C4)CC3C5)c3ccc4c(c3)C(C)(C)CCC4(C)C)ccc21]

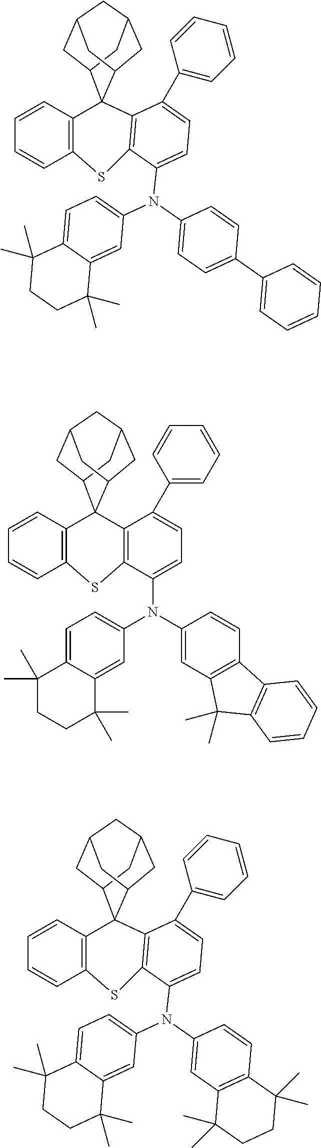

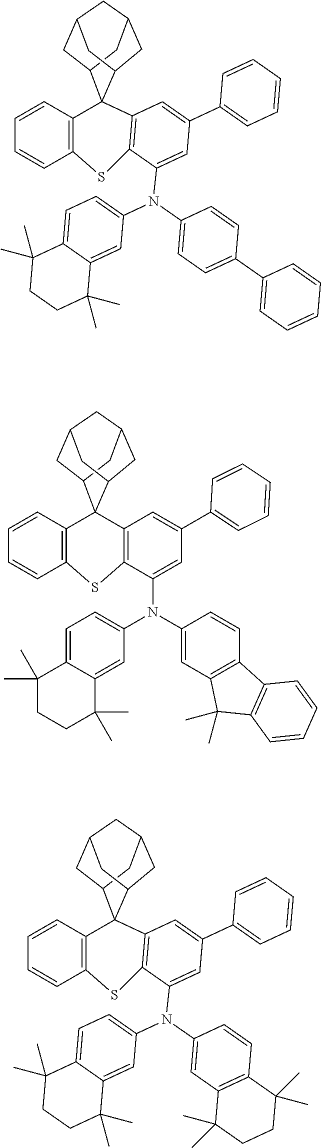

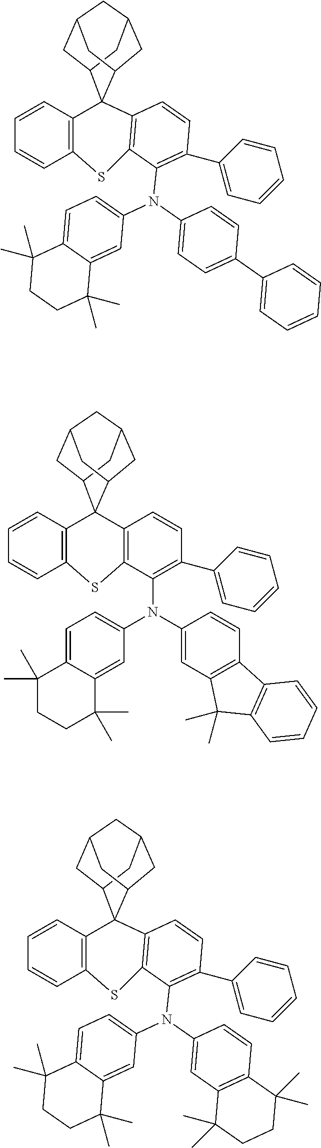

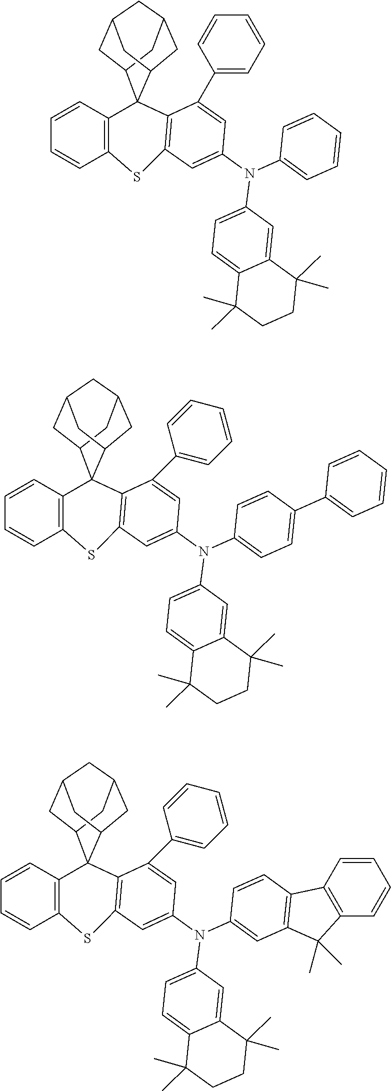 [SMILES: CC1(C)CCC(C)(C)c2cc(N(c3ccccc3)c3cc4c(c(-c5ccccc5)c3)C3(c5ccccc5S4)C4CC5CC(C4)CC3C5)ccc21 ; CC1(C)CCC(C)(C)c2cc(N(c3ccc(-c4ccccc4)cc3)c3cc4c(c(-c5ccccc5)c3)C3(c5ccccc5S4)C4CC5CC(C4)CC3C5)ccc21 ; CC1(C)CCC(C)(C)c2cc(N(c3cc4c(c(-c5ccccc5)c3)C3(c5ccccc5S4)C4CC5CC(C4)CC3C5)c3ccc4c(c3)C(C)(C)c3ccccc3-4)ccc21]

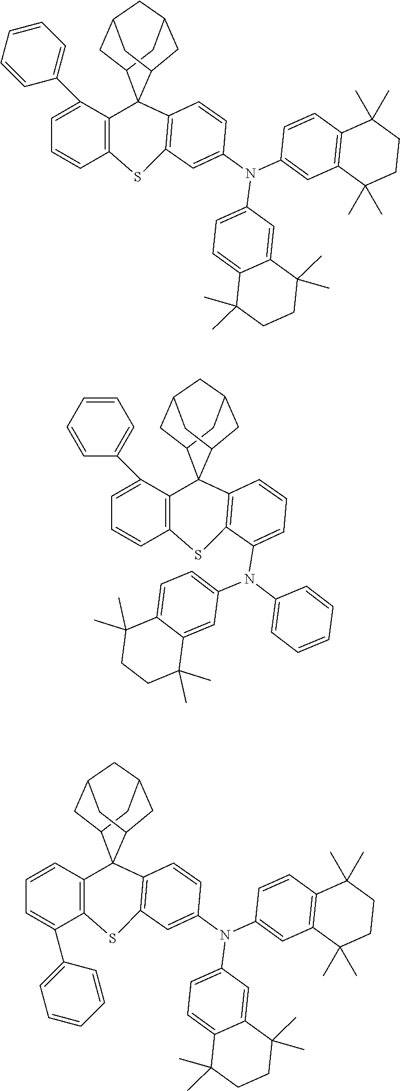 [SMILES: CC1(C)CCC(C)(C)c2cc(N(c3ccc4c(c3)Sc3cccc(-c5ccccc5)c3C43C4CC5CC(C4)CC3C5)c3ccc4c(c3)C(C)(C)CCC4(C)C)ccc21 ; CC1(C)CCC(C)(C)c2cc(N(c3ccccc3)c3cccc4c3Sc3cccc(-c5ccccc5)c3C43C4CC5CC(C4)CC3C5)ccc21 ; CC1(C)CCC(C)(C)c2cc(N(c3ccc4c(c3)Sc3c(-c5ccccc5)cccc3C43C4CC5CC(C4)CC3C5)c3ccc4c(c3)C(C)(C)CCC4(C)C)ccc21]

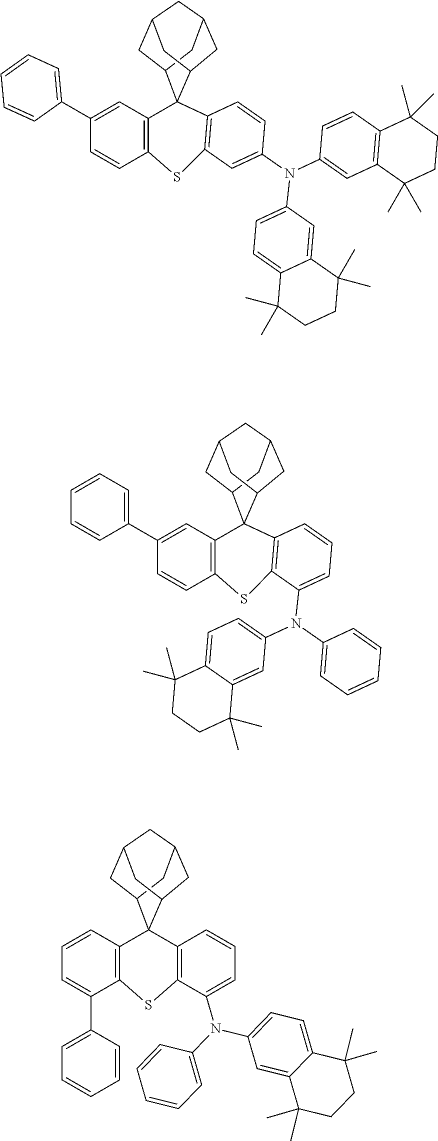 [SMILES: CC1(C)CCC(C)(C)c2cc(N(c3ccc4c(c3)Sc3ccc(-c5ccccc5)cc3C43C4CC5CC(C4)CC3C5)c3ccc4c(c3)C(C)(C)CCC4(C)C)ccc21 ; CC1(C)CCC(C)(C)c2cc(N(c3ccccc3)c3cccc4c3Sc3ccc(-c5ccccc5)cc3C43C4CC5CC(C4)CC3C5)ccc21 ; CC1(C)CCC(C)(C)c2cc(N(c3ccccc3)c3cccc4c3Sc3c(-c5ccccc5)cccc3C43C4CC5CC(C4)CC3C5)ccc21]

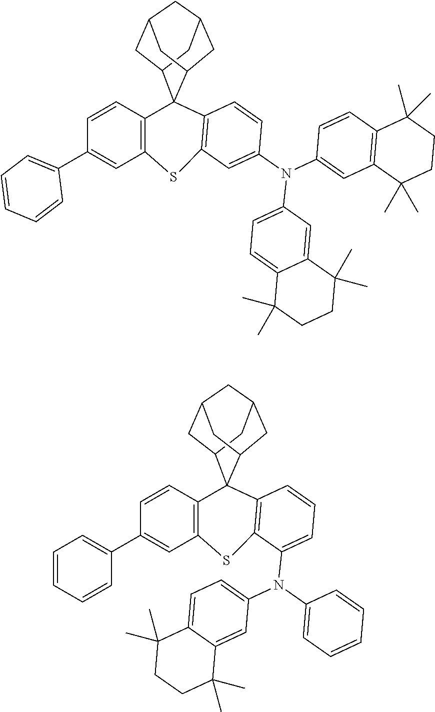 [SMILES: CC1(C)CCC(C)(C)c2cc(N(c3ccc4c(c3)Sc3cc(-c5ccccc5)ccc3C43C4CC5CC(C4)CC3C5)c3ccc4c(c3)C(C)(C)CCC4(C)C)ccc21 ; CC1(C)CCC(C)(C)c2cc(N(c3ccccc3)c3cccc4c3Sc3cc(-c5ccccc5)ccc3C43C4CC5CC(C4)CC3C5)ccc21]

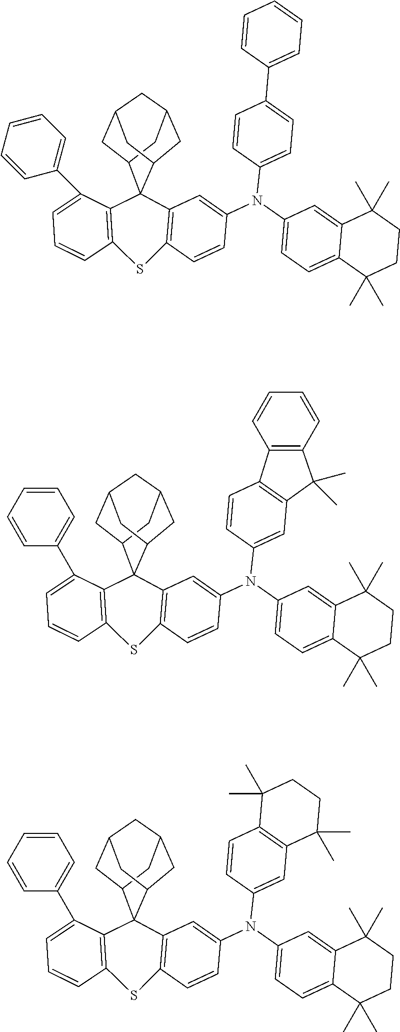 [SMILES: CC1(C)CCC(C)(C)c2cc(N(c3ccc(-c4ccccc4)cc3)c3ccc4c(c3)C3(c5c(cccc5-c5ccccc5)S4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5c(cccc5-c5ccccc5)S4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5c(cccc5-c5ccccc5)S4)C4CC5CC(C4)CC3C5)ccc21]

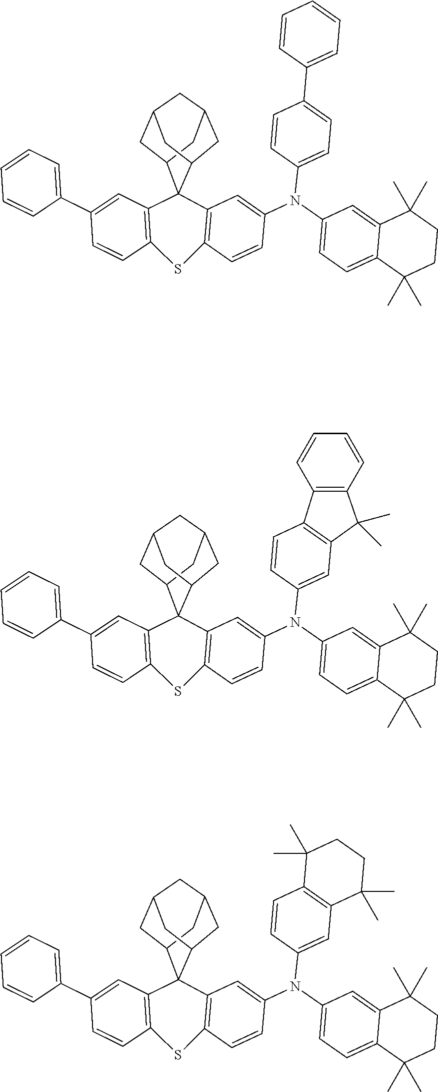 [SMILES: CC1(C)CCC(C)(C)c2cc(N(c3ccc(-c4ccccc4)cc3)c3ccc4c(c3)C3(c5cc(-c6ccccc6)ccc5S4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5cc(-c6ccccc6)ccc5S4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5cc(-c6ccccc6)ccc5S4)C4CC5CC(C4)CC3C5)ccc21]

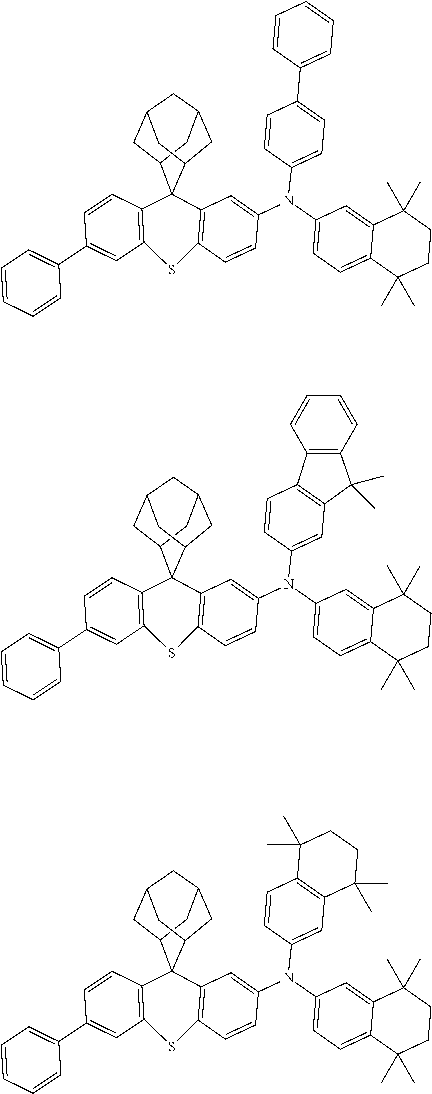 [SMILES: CC1(C)CCC(C)(C)c2cc(N(c3ccc(-c4ccccc4)cc3)c3ccc4c(c3)C3(c5ccc(-c6ccccc6)cc5S4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5ccc(-c6ccccc6)cc5S4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5ccc(-c6ccccc6)cc5S4)C4CC5CC(C4)CC3C5)ccc21]

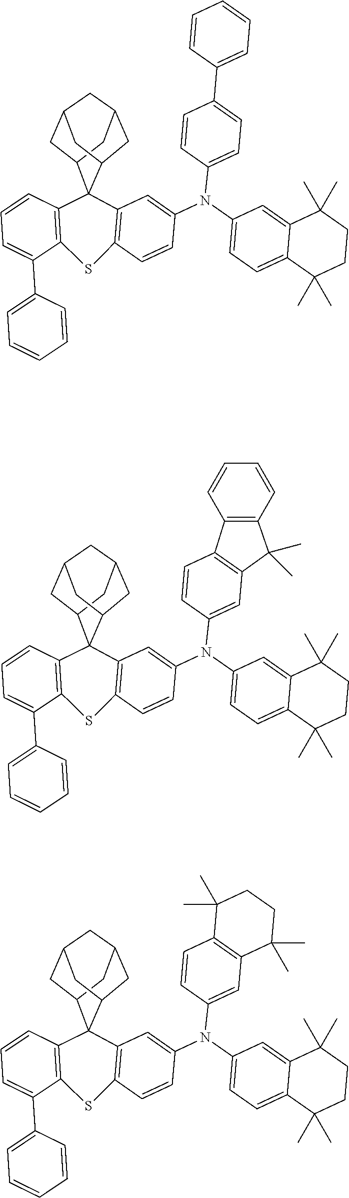 [SMILES: CC1(C)CCC(C)(C)c2cc(N(c3ccc(-c4ccccc4)cc3)c3ccc4c(c3)C3(c5cccc(-c6ccccc6)c5S4)C4CC5CC(C4)CC3C5)ccc21 ; CC1(C)CCC(C)(C)c2cc(N(c3ccc4c(c3)C(C)(C)c3ccccc3-4)c3ccc4c(c3)C3(c5cccc(-c6ccccc6)c5S4)C4CC5CC(C4)CC3C5)ccc21 ; CC1(C)CCC(C)(C)c2cc(N(c3ccc4c(c3)C(C)(C)CCC4(C)C)c3ccc4c(c3)C3(c5cccc(-c6ccccc6)c5S4)C4CC5CC(C4)CC3C5)ccc21]

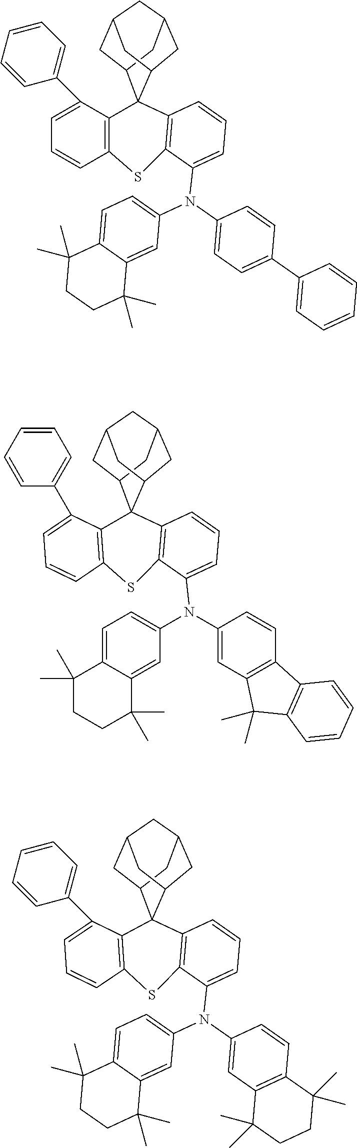 [SMILES: CC1(C)CCC(C)(C)c2cc(N(c3ccc(-c4ccccc4)cc3)c3cccc4c3Sc3cccc(-c5ccccc5)c3C43C4CC5CC(C4)CC3C5)ccc21 ; CC1(C)CCC(C)(C)c2cc(N(c3ccc4c(c3)C(C)(C)c3ccccc3-4)c3cccc4c3Sc3cccc(-c5ccccc5)c3C43C4CC5CC(C4)CC3C5)ccc21 ; CC1(C)CCC(C)(C)c2cc(N(c3ccc4c(c3)C(C)(C)CCC4(C)C)c3cccc4c3Sc3cccc(-c5ccccc5)c3C43C4CC5CC(C4)CC3C5)ccc21]

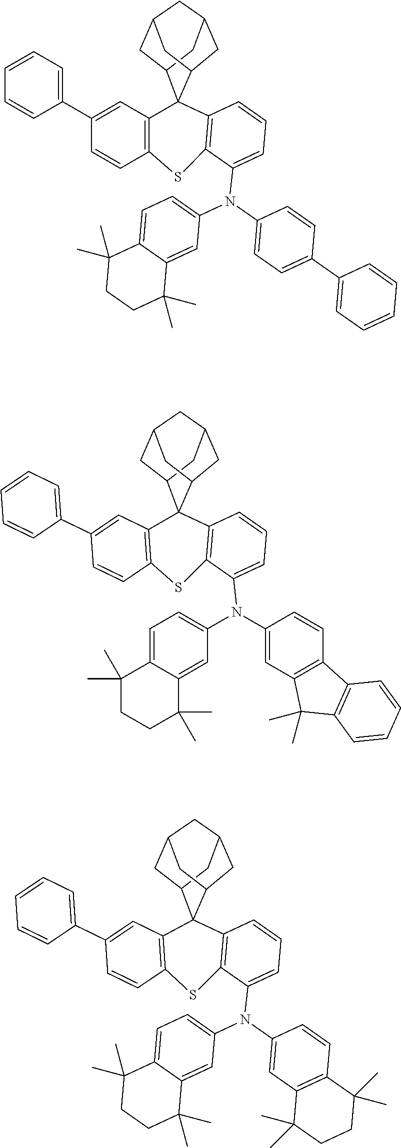 [SMILES: CC1(C)CCC(C)(C)c2cc(N(c3ccc(-c4ccccc4)cc3)c3cccc4c3Sc3ccc(-c5ccccc5)cc3C43C4CC5CC(C4)CC3C5)ccc21 ; CC1(C)CCC(C)(C)c2cc(N(c3ccc4c(c3)C(C)(C)c3ccccc3-4)c3cccc4c3Sc3ccc(-c5ccccc5)cc3C43C4CC5CC(C4)CC3C5)ccc21 ; CC1(C)CCC(C)(C)c2cc(N(c3ccc4c(c3)C(C)(C)CCC4(C)C)c3cccc4c3Sc3ccc(-c5ccccc5)cc3C43C4CC5CC(C4)CC3C5)ccc21]

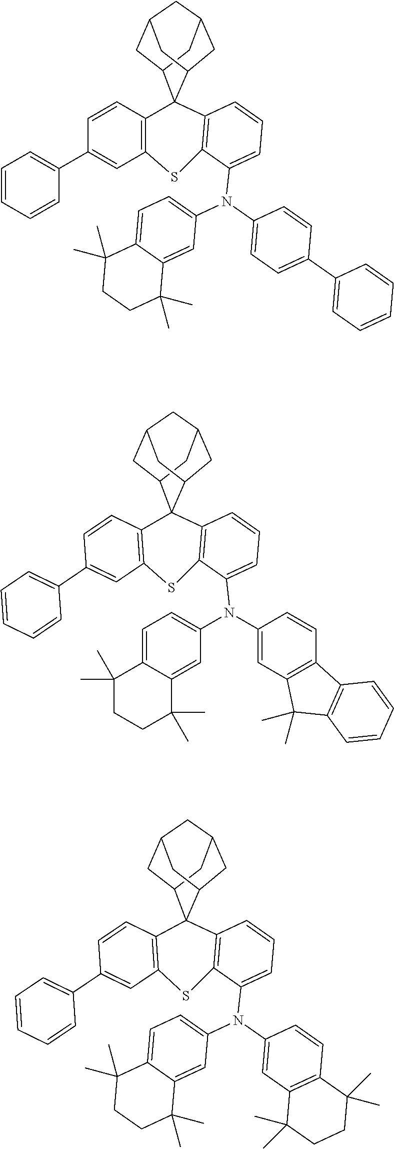 [SMILES: CC1(C)CCC(C)(C)c2cc(N(c3ccc(-c4ccccc4)cc3)c3cccc4c3Sc3cc(-c5ccccc5)ccc3C43C4CC5CC(C4)CC3C5)ccc21 ; CC1(C)CCC(C)(C)c2cc(N(c3ccc4c(c3)C(C)(C)c3ccccc3-4)c3cccc4c3Sc3cc(-c5ccccc5)ccc3C43C4CC5CC(C4)CC3C5)ccc21 ; CC1(C)CCC(C)(C)c2cc(N(c3ccc4c(c3)C(C)(C)CCC4(C)C)c3cccc4c3Sc3cc(-c5ccccc5)ccc3C43C4CC5CC(C4)CC3C5)ccc21]

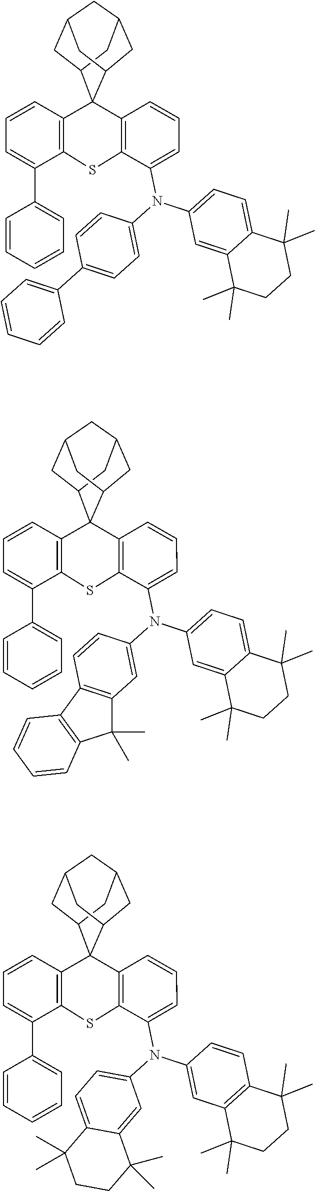 [SMILES: CC1(C)CCC(C)(C)c2cc(N(c3ccc(-c4ccccc4)cc3)c3cccc4c3Sc3c(-c5ccccc5)cccc3C43C4CC5CC(C4)CC3C5)ccc21 ; CC1(C)CCC(C)(C)c2cc(N(c3ccc4c(c3)C(C)(C)c3ccccc3-4)c3cccc4c3Sc3c(-c5ccccc5)cccc3C43C4CC5CC(C4)CC3C5)ccc21 ; CC1(C)CCC(C)(C)c2cc(N(c3ccc4c(c3)C(C)(C)CCC4(C)C)c3cccc4c3Sc3c(-c5ccccc5)cccc3C43C4CC5CC(C4)CC3C5)ccc21]

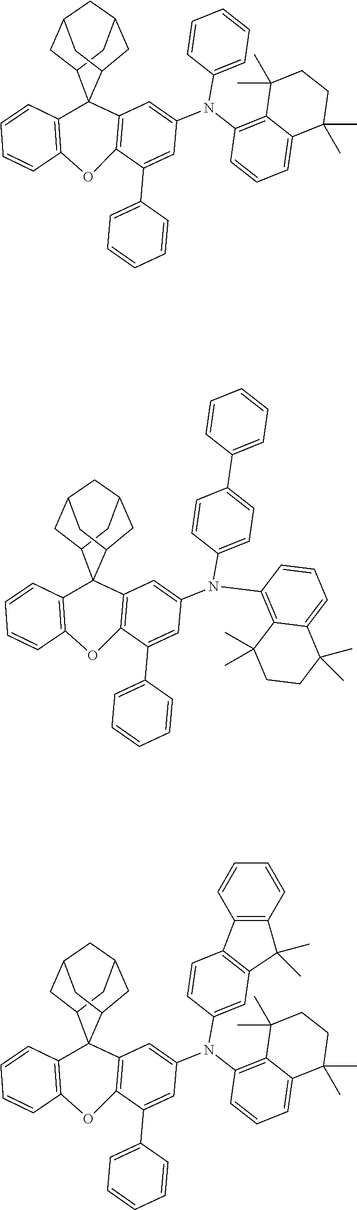 [SMILES: CC1(C)CCC(C)(C)c2c(N(c3ccccc3)c3cc(-c4ccccc4)c4c(c3)C3(c5ccccc5O4)C4CC5CC(C4)CC3C5)cccc21 ; CC1(C)CCC(C)(C)c2c(N(c3ccc(-c4ccccc4)cc3)c3cc(-c4ccccc4)c4c(c3)C3(c5ccccc5O4)C4CC5CC(C4)CC3C5)cccc21 ; CC1(C)CCC(C)(C)c2c(N(c3ccc4c(c3)C(C)(C)c3ccccc3-4)c3cc(-c4ccccc4)c4c(c3)C3(c5ccccc5O4)C4CC5CC(C4)CC3C5)cccc21]

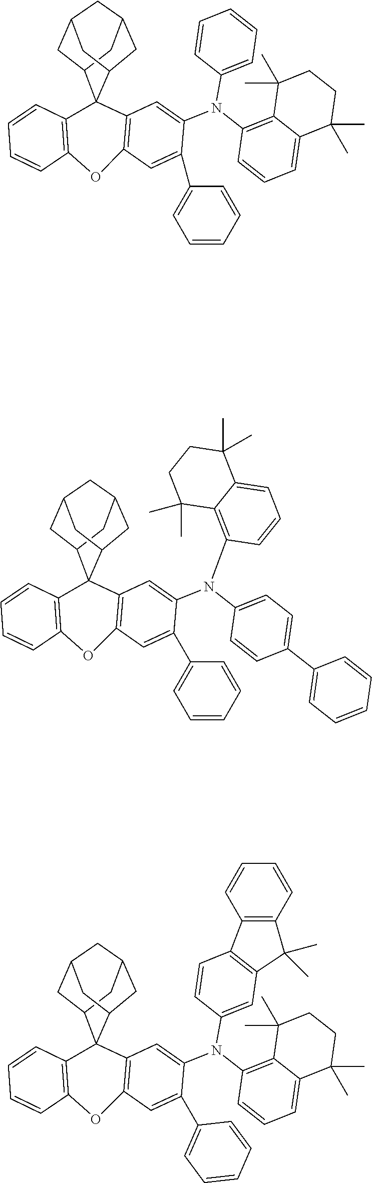 [SMILES: CC1(C)CCC(C)(C)c2c(N(c3ccccc3)c3cc4c(cc3-c3ccccc3)Oc3ccccc3C43C4CC5CC(C4)CC3C5)cccc21 ; CC1(C)CCC(C)(C)c2c(N(c3ccc(-c4ccccc4)cc3)c3cc4c(cc3-c3ccccc3)Oc3ccccc3C43C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)Oc3ccccc3C43C4CC5CC(C4)CC3C5)cccc21]

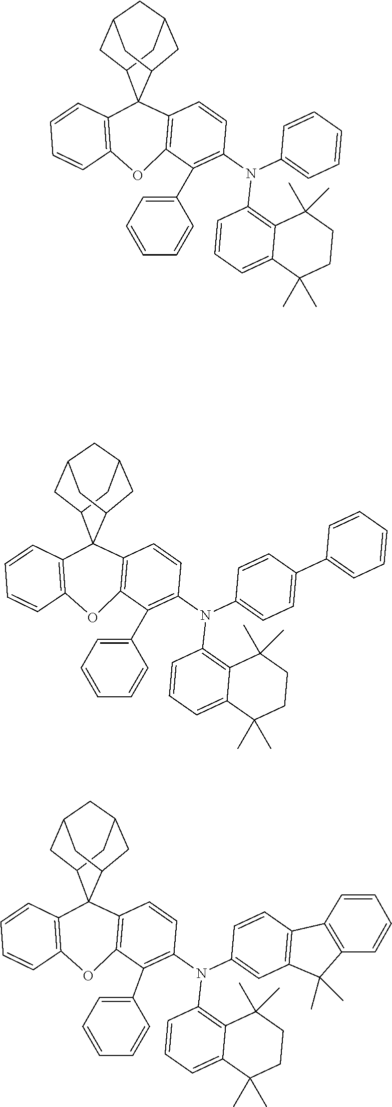 [SMILES: CC1(C)CCC(C)(C)c2c(N(c3ccccc3)c3ccc4c(c3-c3ccccc3)Oc3ccccc3C43C4CC5CC(C4)CC3C5)cccc21 ; CC1(C)CCC(C)(C)c2c(N(c3ccc(-c4ccccc4)cc3)c3ccc4c(c3-c3ccccc3)Oc3ccccc3C43C4CC5CC(C4)CC3C5)cccc21 ; CC1(C)CCC(C)(C)c2c(N(c3ccc4c(c3)C(C)(C)c3ccccc3-4)c3ccc4c(c3-c3ccccc3)Oc3ccccc3C43C4CC5CC(C4)CC3C5)cccc21]

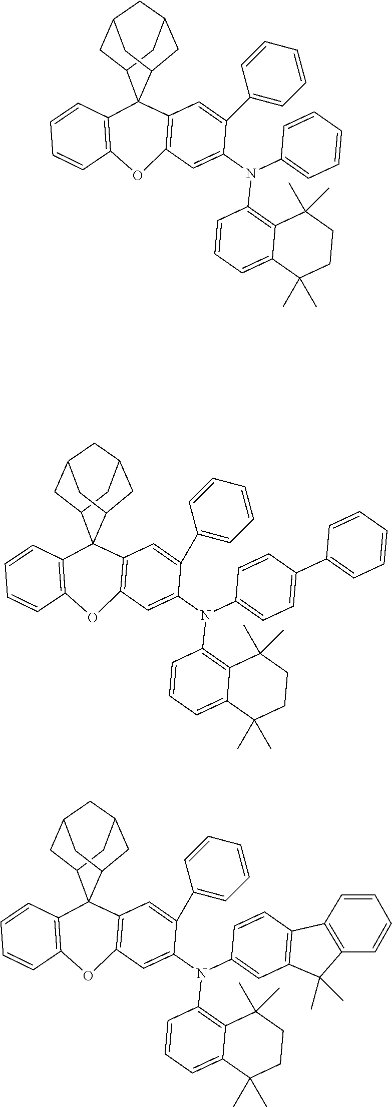 [SMILES: CC1(C)CCC(C)(C)c2c(N(c3ccccc3)c3cc4c(cc3-c3ccccc3)C3(c5ccccc5O4)C4CC5CC(C4)CC3C5)cccc21 ; CC1(C)CCC(C)(C)c2c(N(c3ccc(-c4ccccc4)cc3)c3cc4c(cc3-c3ccccc3)C3(c5ccccc5O4)C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)C3(c5ccccc5O4)C4CC5CC(C4)CC3C5)cccc21]

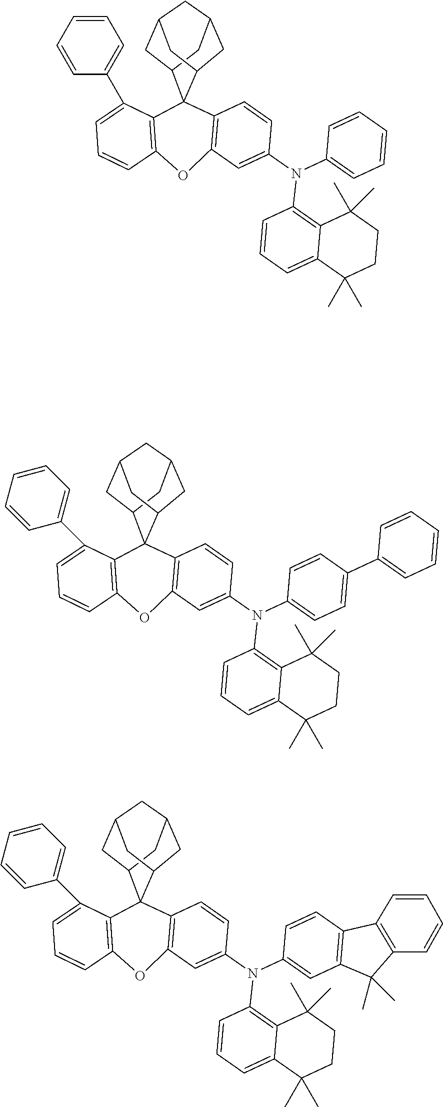 [SMILES: CC1(C)CCC(C)(C)c2c(N(c3ccccc3)c3ccc4c(c3)Oc3cccc(-c5ccccc5)c3C43C4CC5CC(C4)CC3C5)cccc21 ; CC1(C)CCC(C)(C)c2c(N(c3ccc(-c4ccccc4)cc3)c3ccc4c(c3)Oc3cccc(-c5ccccc5)c3C43C4CC5CC(C4)CC3C5)cccc21 ; CC1(C)CCC(C)(C)c2c(N(c3ccc4c(c3)Oc3cccc(-c5ccccc5)c3C43C4CC5CC(C4)CC3C5)c3ccc4c(c3)C(C)(C)c3ccccc3-4)cccc21]

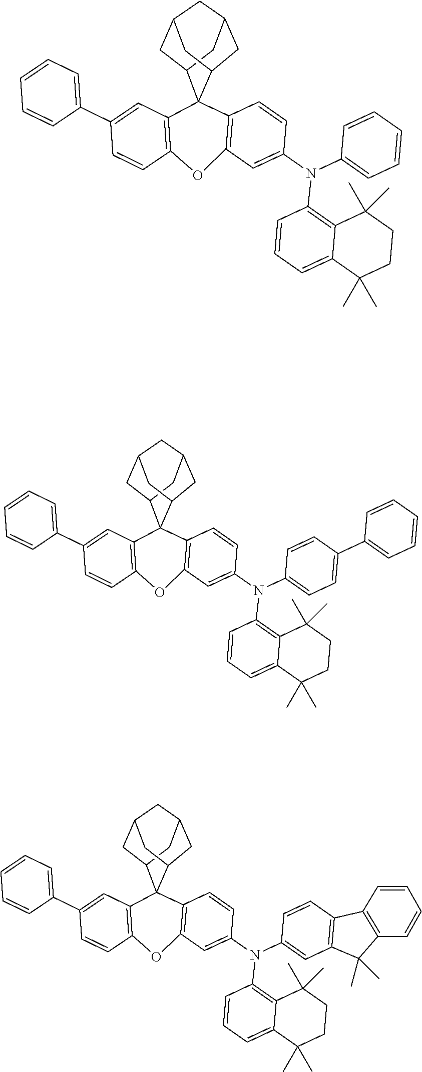 [SMILES: CC1(C)CCC(C)(C)c2c(N(c3ccccc3)c3ccc4c(c3)Oc3ccc(-c5ccccc5)cc3C43C4CC5CC(C4)CC3C5)cccc21 ; CC1(C)CCC(C)(C)c2c(N(c3ccc(-c4ccccc4)cc3)c3ccc4c(c3)Oc3ccc(-c5ccccc5)cc3C43C4CC5CC(C4)CC3C5)cccc21 ; CC1(C)CCC(C)(C)c2c(N(c3ccc4c(c3)Oc3ccc(-c5ccccc5)cc3C43C4CC5CC(C4)CC3C5)c3ccc4c(c3)C(C)(C)c3ccccc3-4)cccc21]

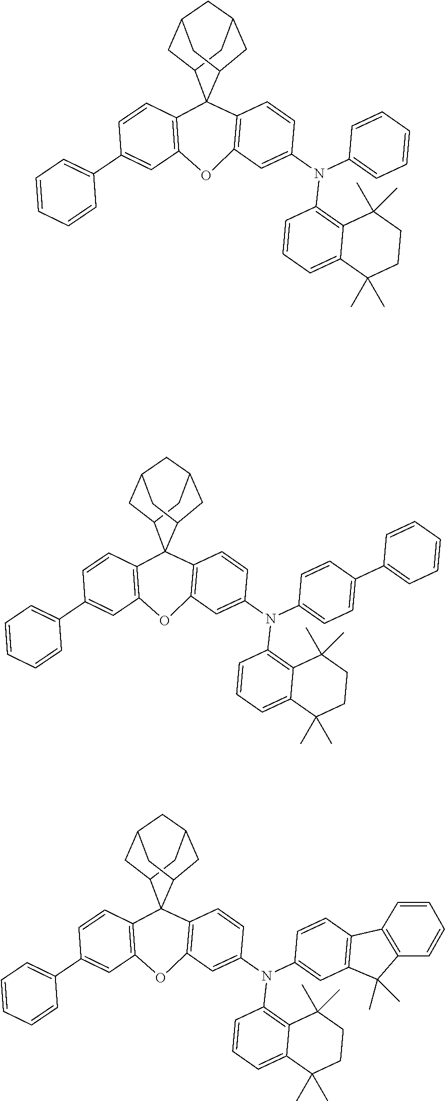 [SMILES: CC1(C)CCC(C)(C)c2c(N(c3ccccc3)c3ccc4c(c3)Oc3cc(-c5ccccc5)ccc3C43C4CC5CC(C4)CC3C5)cccc21 ; CC1(C)CCC(C)(C)c2c(N(c3ccc(-c4ccccc4)cc3)c3ccc4c(c3)Oc3cc(-c5ccccc5)ccc3C43C4CC5CC(C4)CC3C5)cccc21 ; CC1(C)CCC(C)(C)c2c(N(c3ccc4c(c3)Oc3cc(-c5ccccc5)ccc3C43C4CC5CC(C4)CC3C5)c3ccc4c(c3)C(C)(C)c3ccccc3-4)cccc21]

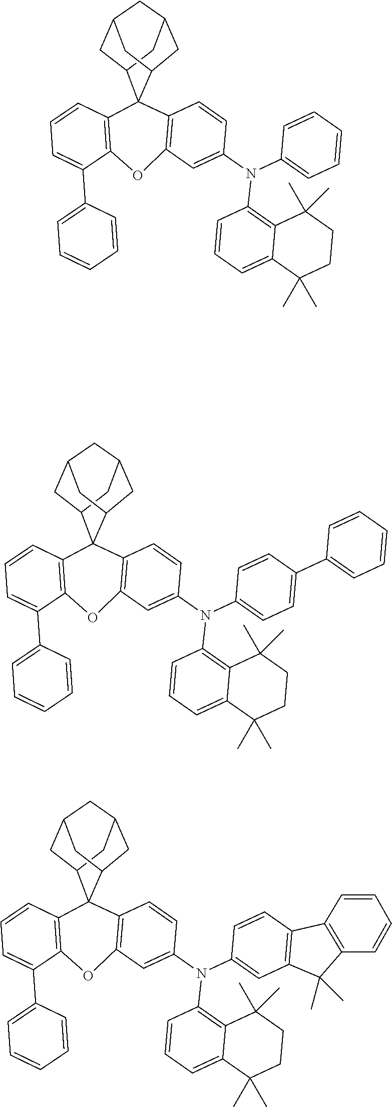 [SMILES: CC1(C)CCC(C)(C)c2c(N(c3ccccc3)c3ccc4c(c3)Oc3c(-c5ccccc5)cccc3C43C4CC5CC(C4)CC3C5)cccc21 ; CC1(C)CCC(C)(C)c2c(N(c3ccc(-c4ccccc4)cc3)c3ccc4c(c3)Oc3c(-c5ccccc5)cccc3C43C4CC5CC(C4)CC3C5)cccc21 ; CC1(C)CCC(C)(C)c2c(N(c3ccc4c(c3)Oc3c(-c5ccccc5)cccc3C43C4CC5CC(C4)CC3C5)c3ccc4c(c3)C(C)(C)c3ccccc3-4)cccc21]

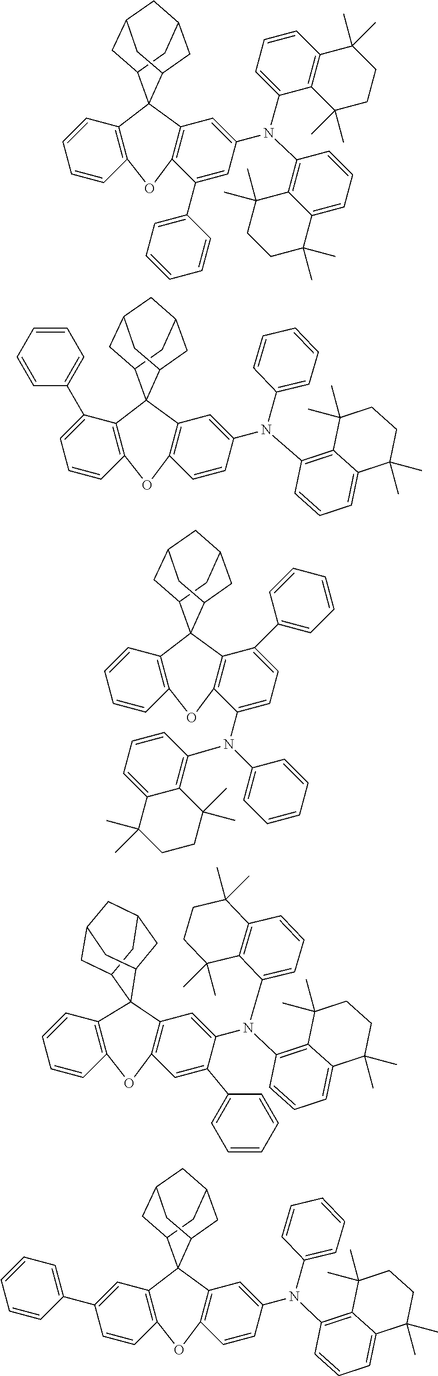

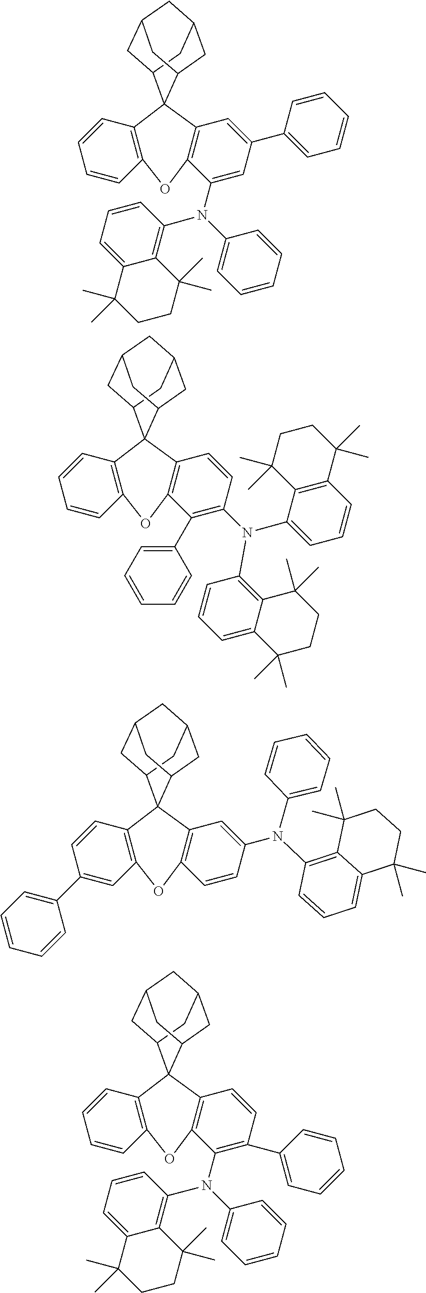

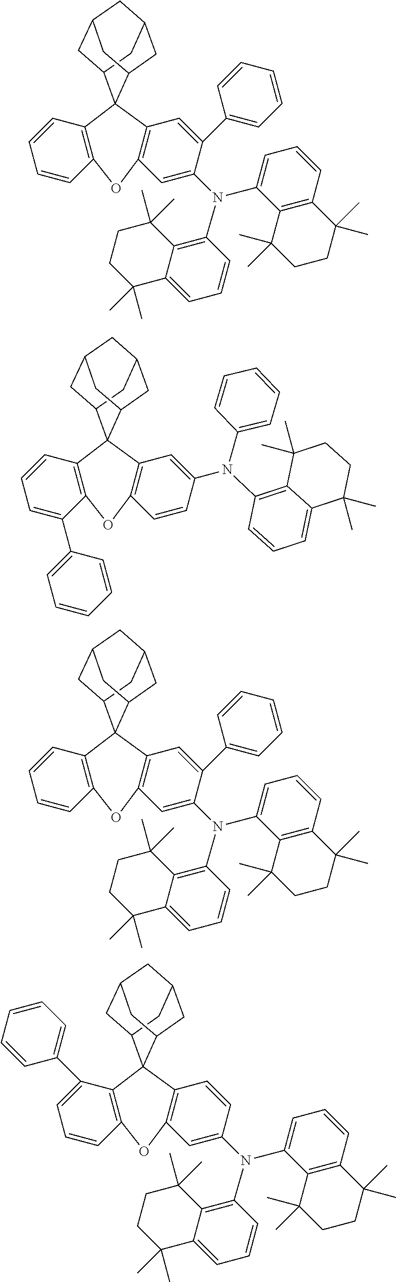

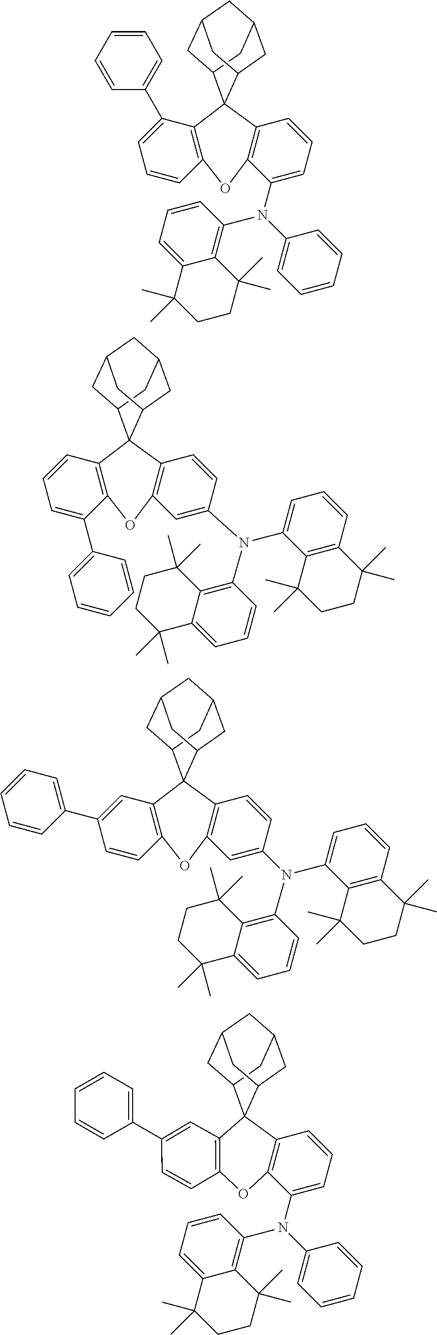

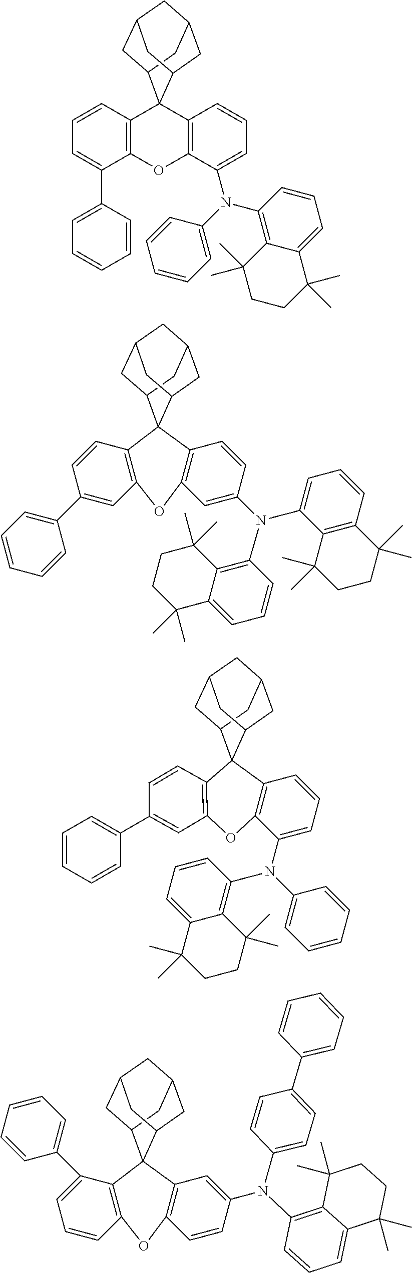

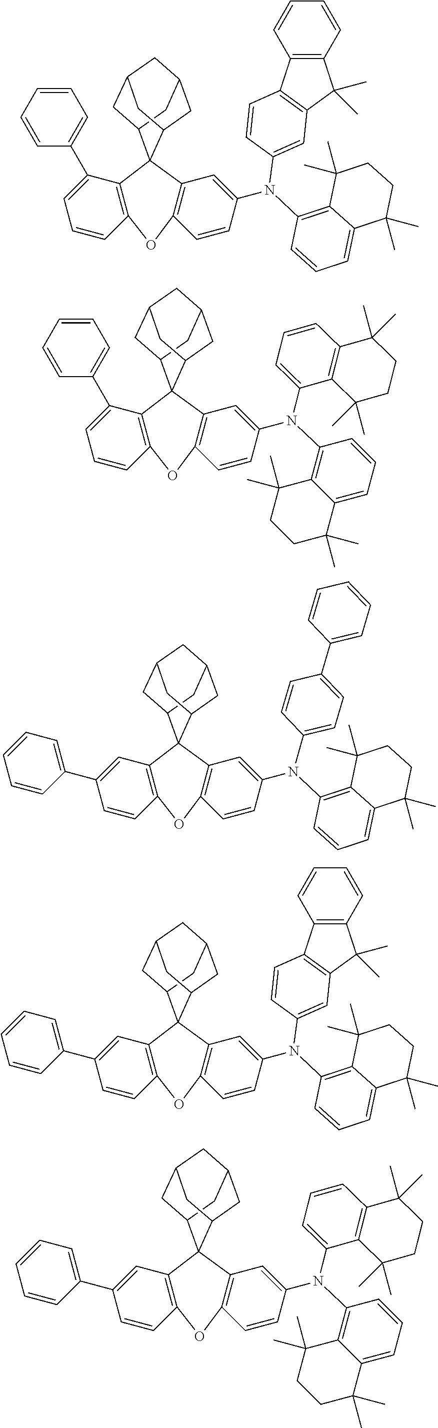

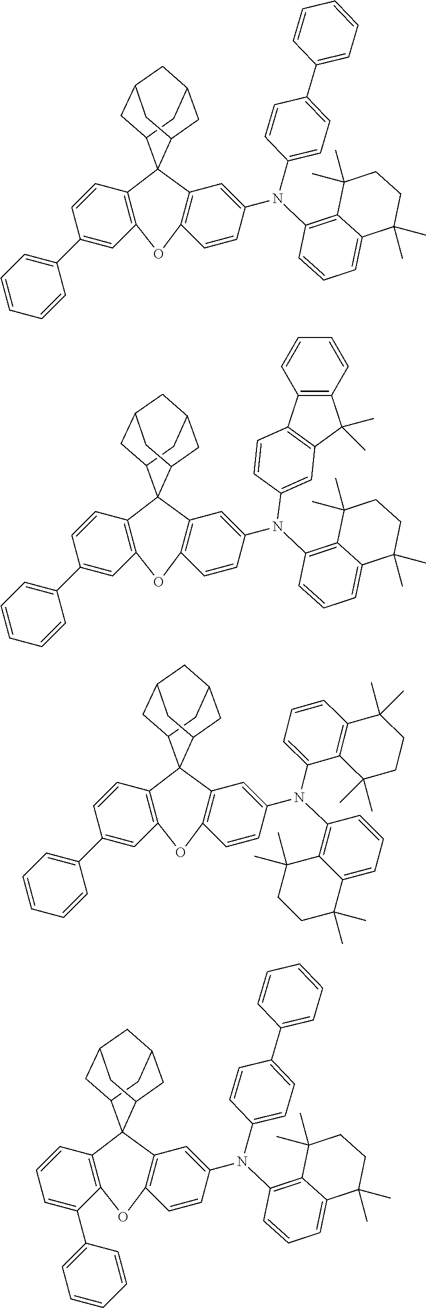

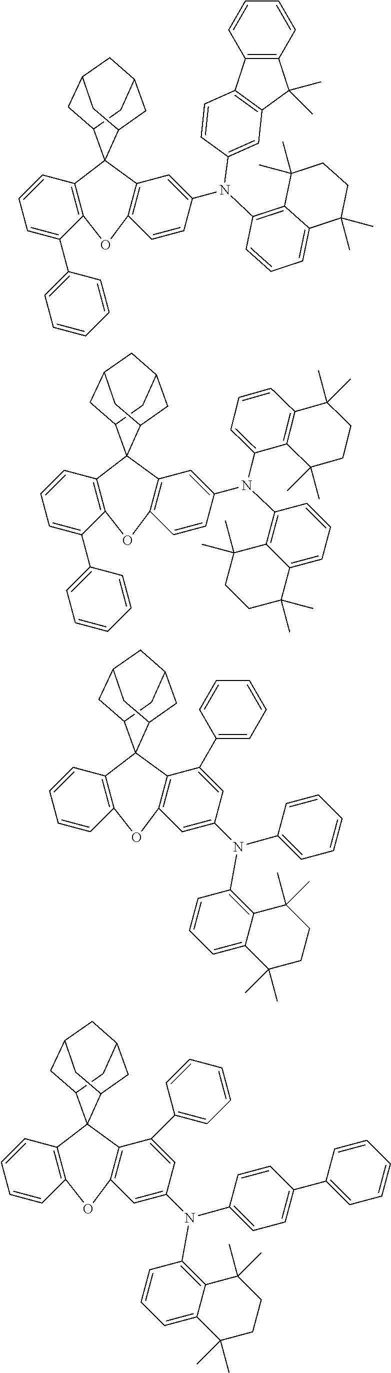

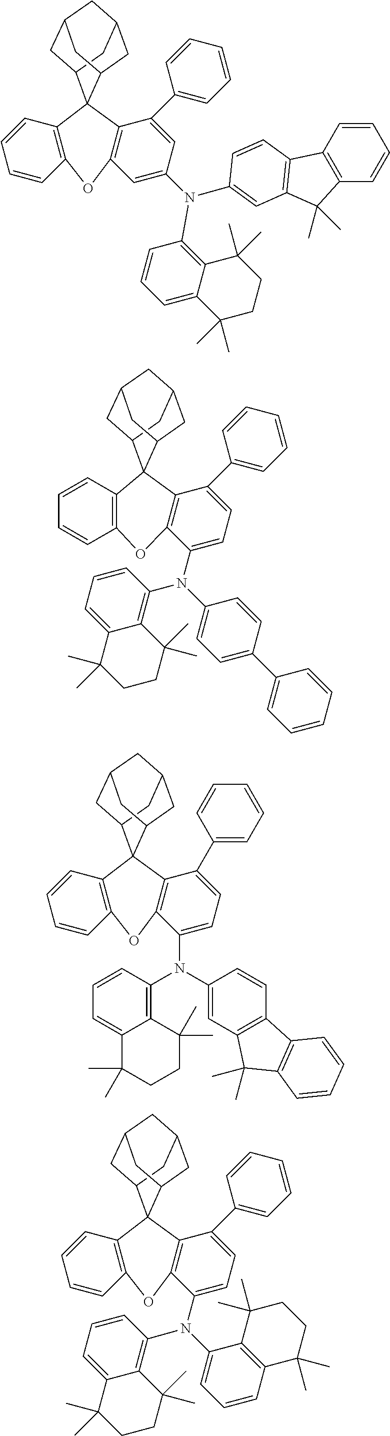

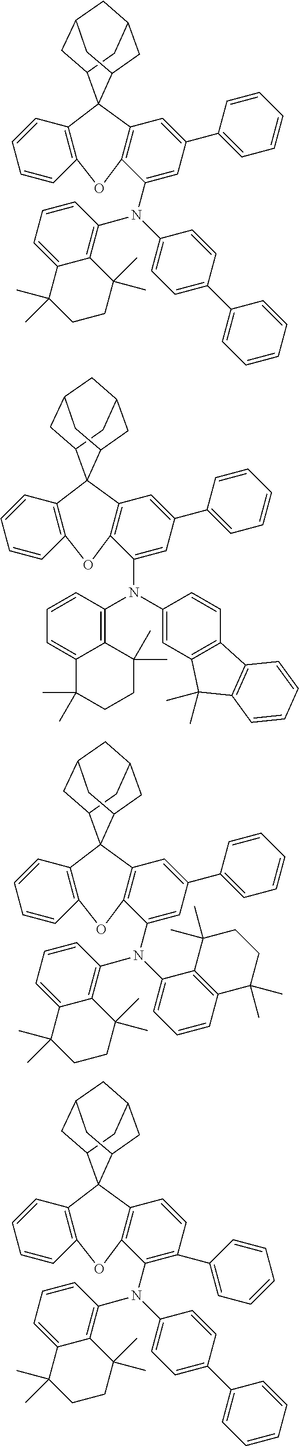

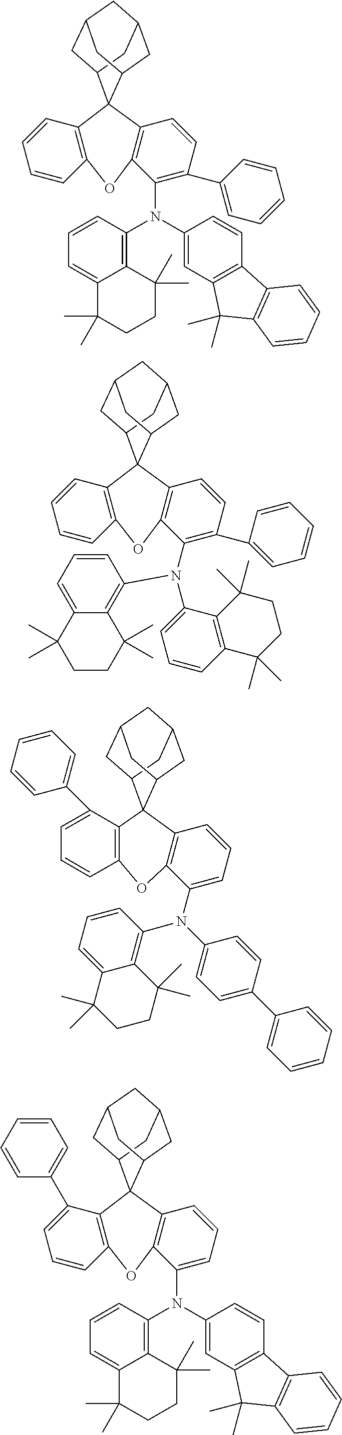

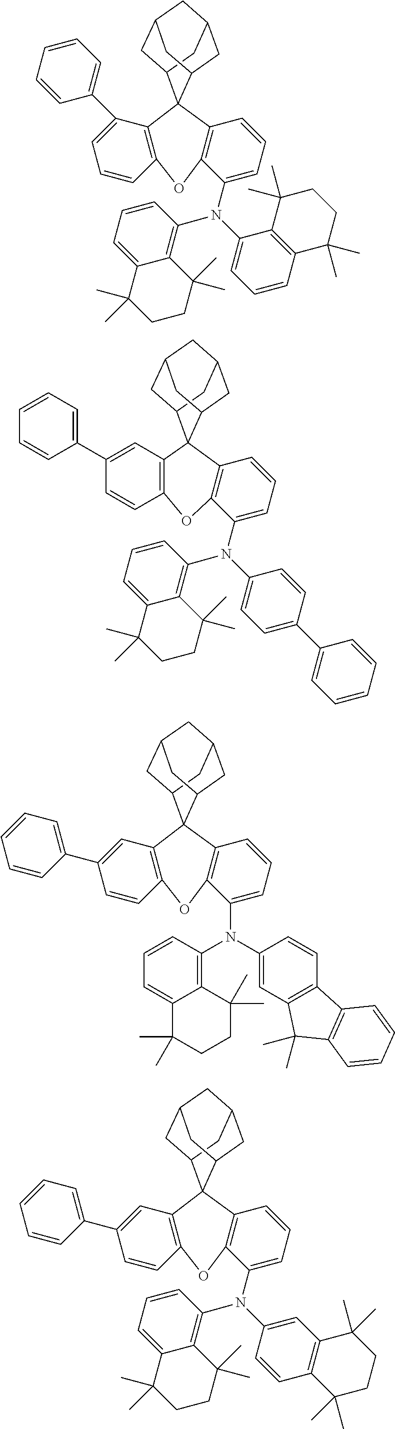

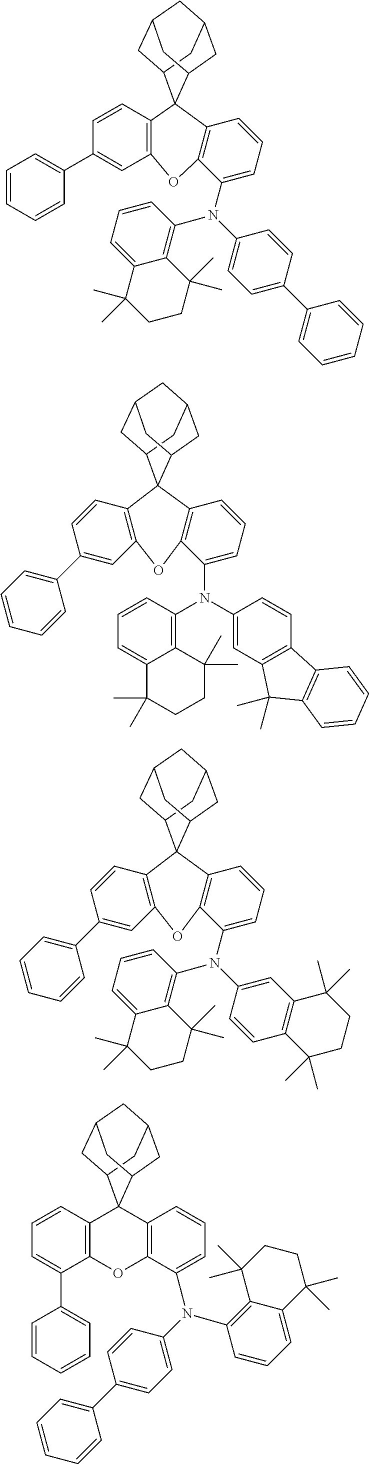

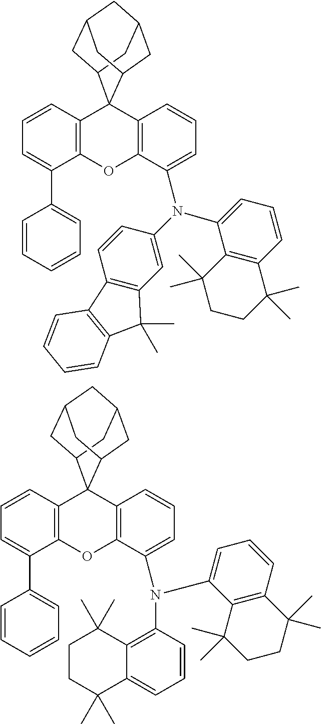

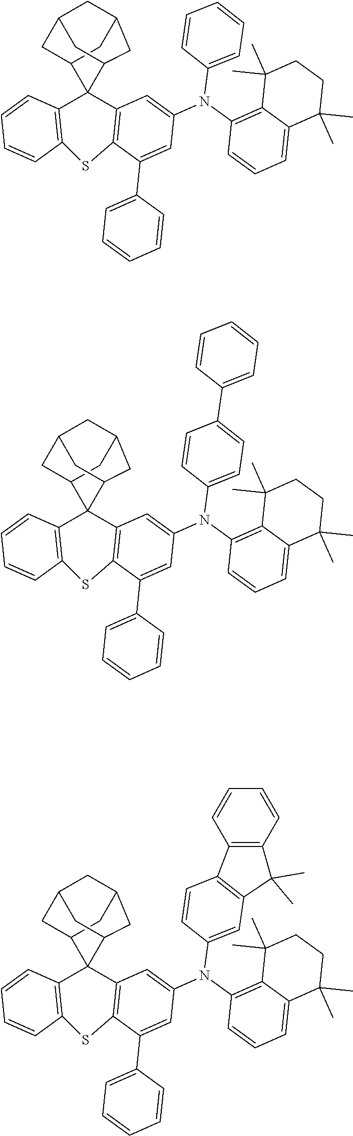 [SMILES: CC1(C)CCC(C)(C)c2c(N(c3ccccc3)c3cc(-c4ccccc4)c4c(c3)C3(c5ccccc5S4)C4CC5CC(C4)CC3C5)cccc21 ; CC1(C)CCC(C)(C)c2c(N(c3ccc(-c4ccccc4)cc3)c3cc(-c4ccccc4)c4c(c3)C3(c5ccccc5S4)C4CC5CC(C4)CC3C5)cccc21 ; CC1(C)CCC(C)(C)c2c(N(c3ccc4c(c3)C(C)(C)c3ccccc3-4)c3cc(-c4ccccc4)c4c(c3)C3(c5ccccc5S4)C4CC5CC(C4)CC3C5)cccc21]

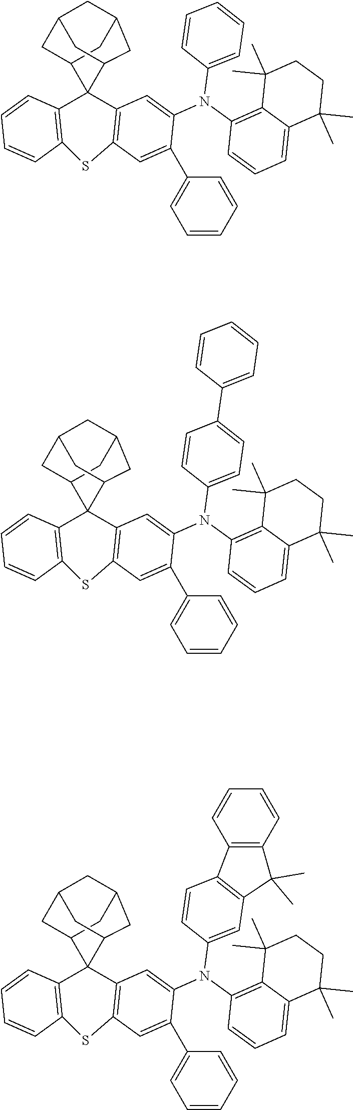 [SMILES: CC1(C)CCC(C)(C)c2c(N(c3ccccc3)c3cc4c(cc3-c3ccccc3)Sc3ccccc3C43C4CC5CC(C4)CC3C5)cccc21 ; CC1(C)CCC(C)(C)c2c(N(c3ccc(-c4ccccc4)cc3)c3cc4c(cc3-c3ccccc3)Sc3ccccc3C43C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)Sc3ccccc3C43C4CC5CC(C4)CC3C5)cccc21]

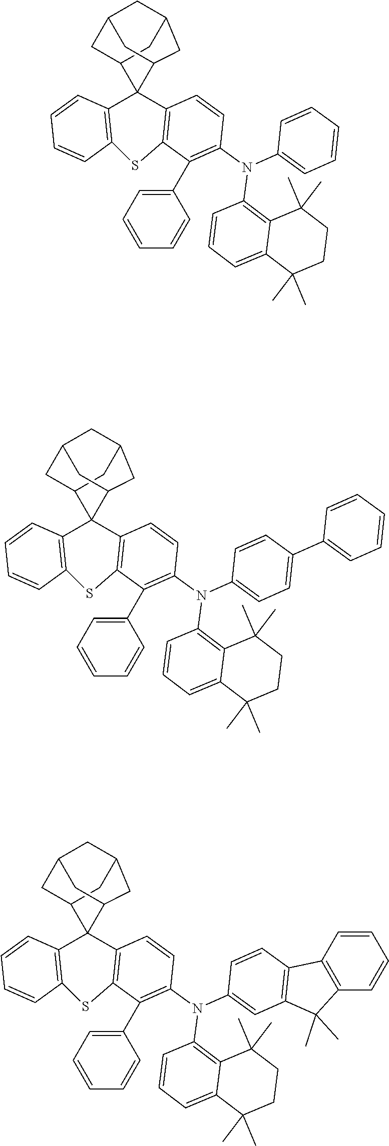 [SMILES: CC1(C)CCC(C)(C)c2c(N(c3ccccc3)c3ccc4c(c3-c3ccccc3)Sc3ccccc3C43C4CC5CC(C4)CC3C5)cccc21 ; CC1(C)CCC(C)(C)c2c(N(c3ccc(-c4ccccc4)cc3)c3ccc4c(c3-c3ccccc3)Sc3ccccc3C43C4CC5CC(C4)CC3C5)cccc21 ; CC1(C)CCC(C)(C)c2c(N(c3ccc4c(c3)C(C)(C)c3ccccc3-4)c3ccc4c(c3-c3ccccc3)Sc3ccccc3C43C4CC5CC(C4)CC3C5)cccc21]

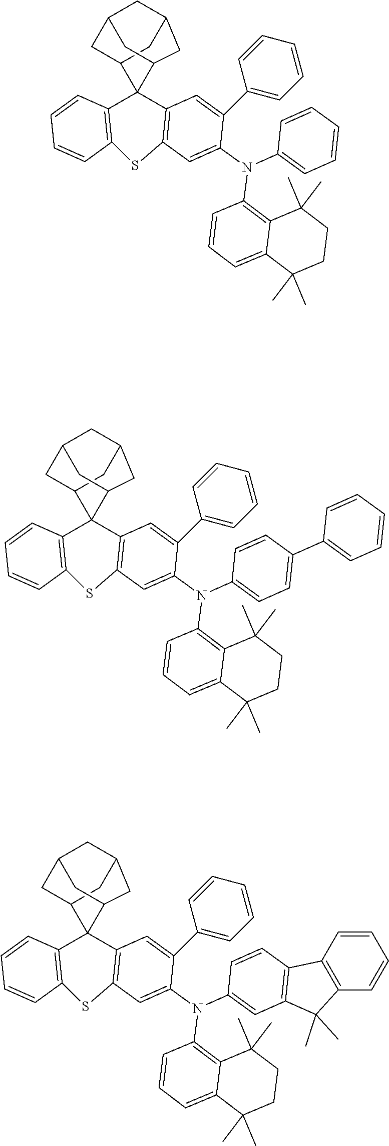 [SMILES: CC1(C)CCC(C)(C)c2c(N(c3ccccc3)c3cc4c(cc3-c3ccccc3)C3(c5ccccc5S4)C4CC5CC(C4)CC3C5)cccc21 ; CC1(C)CCC(C)(C)c2c(N(c3ccc(-c4ccccc4)cc3)c3cc4c(cc3-c3ccccc3)C3(c5ccccc5S4)C4CC5CC(C4)CC3C5)cccc21 ; CC1(C)CCC(C)(C)c2c(N(c3ccc4c(c3)C(C)(C)c3ccccc3-4)c3cc4c(cc3-c3ccccc3)C3(c5ccccc5S4)C4CC5CC(C4)CC3C5)cccc21]

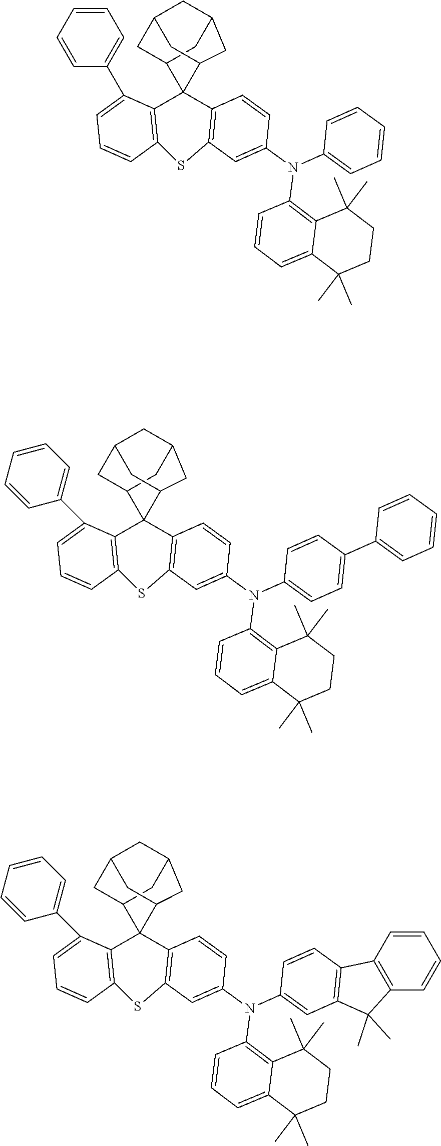 [SMILES: CC1(C)CCC(C)(C)c2c(N(c3ccccc3)c3ccc4c(c3)Sc3cccc(-c5ccccc5)c3C43C4CC5CC(C4)CC3C5)cccc21 ; CC1(C)CCC(C)(C)c2c(N(c3ccc(-c4ccccc4)cc3)c3ccc4c(c3)Sc3cccc(-c5ccccc5)c3C43C4CC5CC(C4)CC3C5)cccc21 ; CC1(C)CCC(C)(C)c2c(N(c3ccc4c(c3)Sc3cccc(-c5ccccc5)c3C43C4CC5CC(C4)CC3C5)c3ccc4c(c3)C(C)(C)c3ccccc3-4)cccc21]

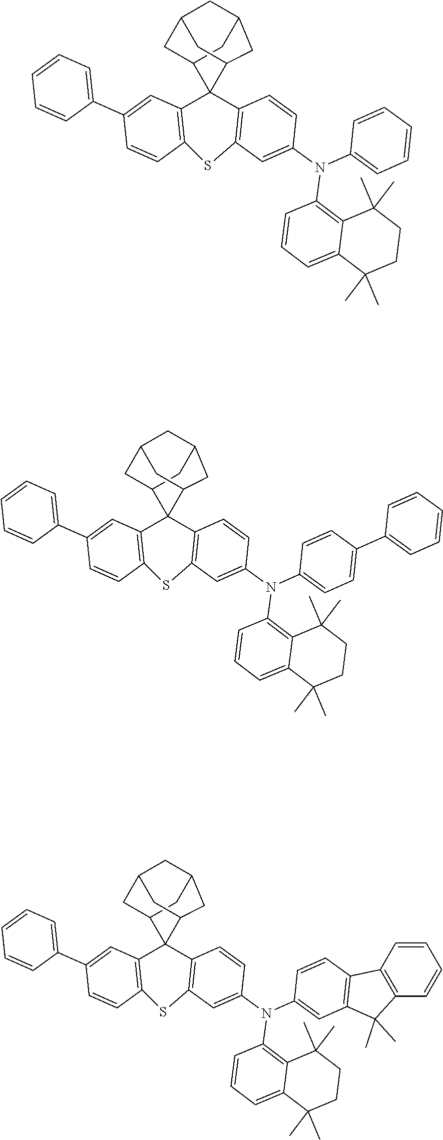 [SMILES: CC1(C)CCC(C)(C)c2c(N(c3ccccc3)c3ccc4c(c3)Sc3ccc(-c5ccccc5)cc3C43C4CC5CC(C4)CC3C5)cccc21 ; CC1(C)CCC(C)(C)c2c(N(c3ccc(-c4ccccc4)cc3)c3ccc4c(c3)Sc3ccc(-c5ccccc5)cc3C43C4CC5CC(C4)CC3C5)cccc21 ; CC1(C)CCC(C)(C)c2c(N(c3ccc4c(c3)Sc3ccc(-c5ccccc5)cc3C43C4CC5CC(C4)CC3C5)c3ccc4c(c3)C(C)(C)c3ccccc3-4)cccc21]

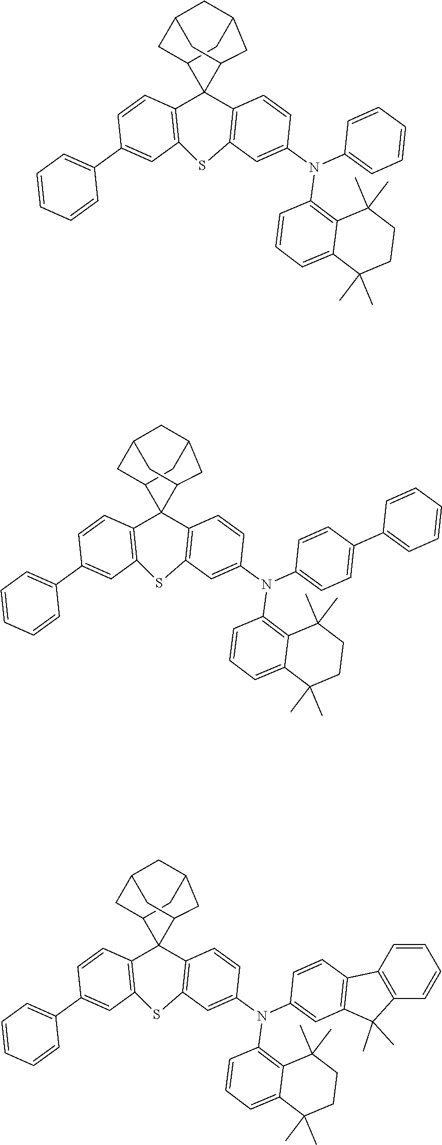 [SMILES: CC1(C)CCC(C)(C)c2c(N(c3ccccc3)c3ccc4c(c3)Sc3cc(-c5ccccc5)ccc3C43C4CC5CC(C4)CC3C5)cccc21 ; CC1(C)CCC(C)(C)c2c(N(c3ccc(-c4ccccc4)cc3)c3ccc4c(c3)Sc3cc(-c5ccccc5)ccc3C43C4CC5CC(C4)CC3C5)cccc21 ; CC1(C)CCC(C)(C)c2c(N(c3ccc4c(c3)Sc3cc(-c5ccccc5)ccc3C43C4CC5CC(C4)CC3C5)c3ccc4c(c3)C(C)(C)c3ccccc3-4)cccc21]

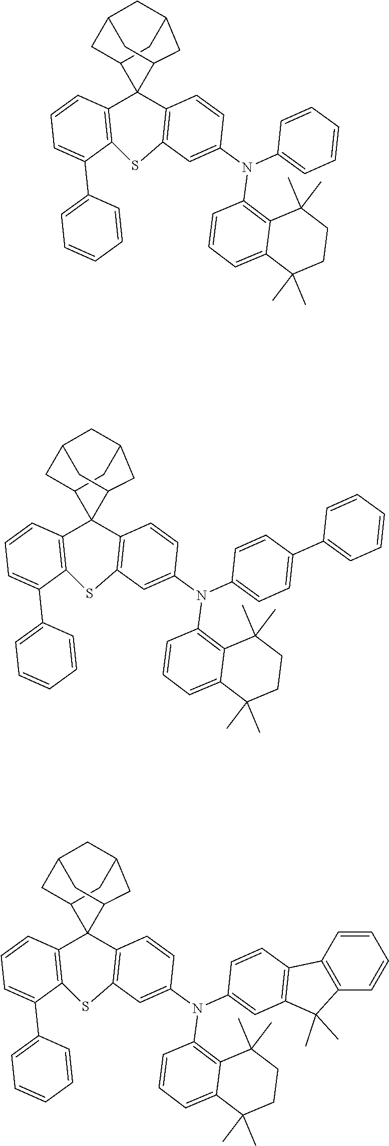 [SMILES: CC1(C)CCC(C)(C)c2c(N(c3ccccc3)c3ccc4c(c3)Sc3c(-c5ccccc5)cccc3C43C4CC5CC(C4)CC3C5)cccc21 ; CC1(C)CCC(C)(C)c2c(N(c3ccc(-c4ccccc4)cc3)c3ccc4c(c3)Sc3c(-c5ccccc5)cccc3C43C4CC5CC(C4)CC3C5)cccc21 ; CC1(C)CCC(C)(C)c2c(N(c3ccc4c(c3)Sc3c(-c5ccccc5)cccc3C43C4CC5CC(C4)CC3C5)c3ccc4c(c3)C(C)(C)c3ccccc3-4)cccc21]

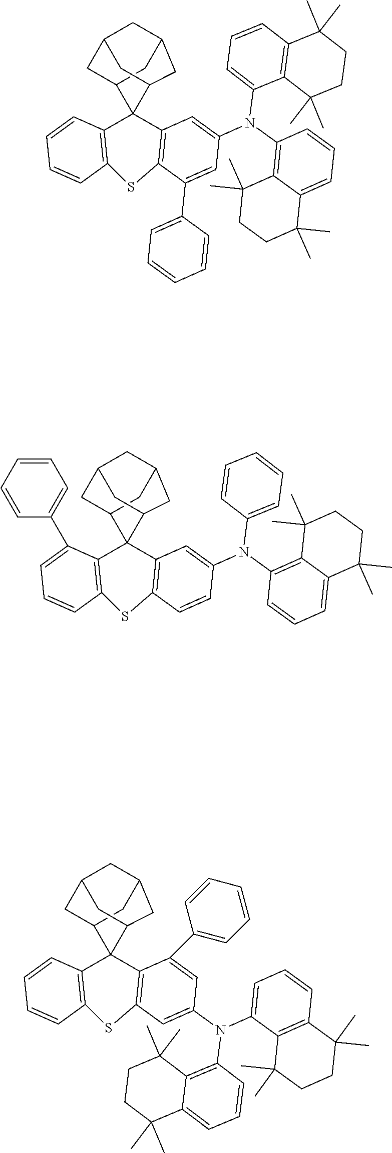 [SMILES: CC1(C)CCC(C)(C)c2c(N(c3cc(-c4ccccc4)c4c(c3)C3(c5ccccc5S4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5c(cccc5-c5ccccc5)S4)C4CC5CC(C4)CC3C5)cccc21 ; CC1(C)CCC(C)(C)c2c(N(c3cc4c(c(-c5ccccc5)c3)C3(c5ccccc5S4)C4CC5CC(C4)CC3C5)c3cccc4c3C(C)(C)CCC4(C)C)cccc21]

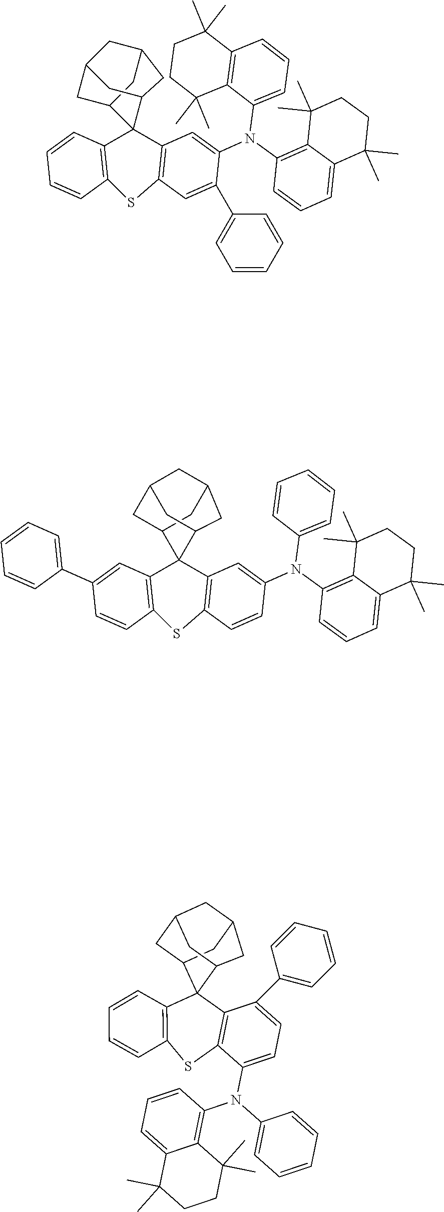 [SMILES: CC1(C)CCC(C)(C)c2c(N(c3cc4c(cc3-c3ccccc3)Sc3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cc(-c6ccccc6)ccc5S4)C4CC5CC(C4)CC3C5)cccc21 ; CC1(C)CCC(C)(C)c2c(N(c3ccccc3)c3ccc(-c4ccccc4)c4c3Sc3ccccc3C43C4CC5CC(C4)CC3C5)cccc21]

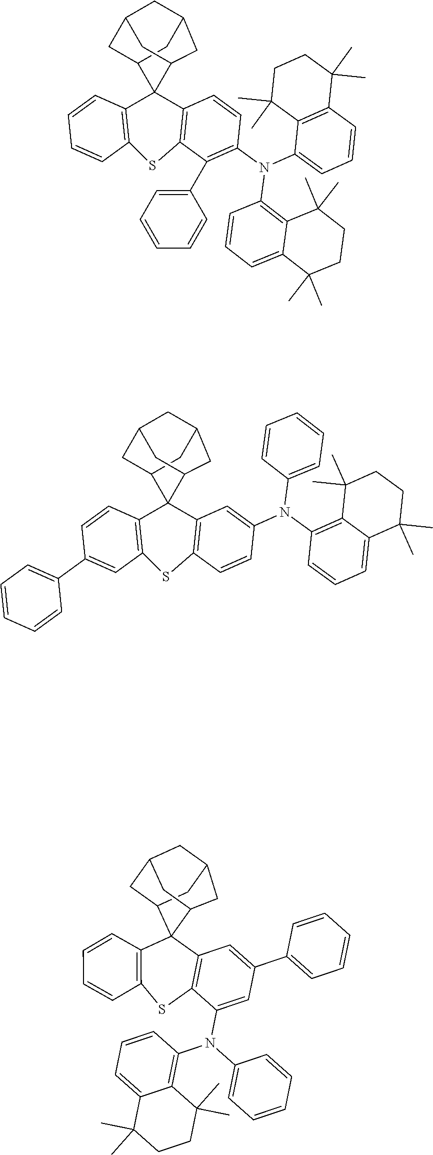 [SMILES: CC1(C)CCC(C)(C)c2c(N(c3ccc4c(c3-c3ccccc3)Sc3ccccc3C43C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5ccc(-c6ccccc6)cc5S4)C4CC5CC(C4)CC3C5)cccc21 ; CC1(C)CCC(C)(C)c2c(N(c3ccccc3)c3cc(-c4ccccc4)cc4c3Sc3ccccc3C43C4CC5CC(C4)CC3C5)cccc21]

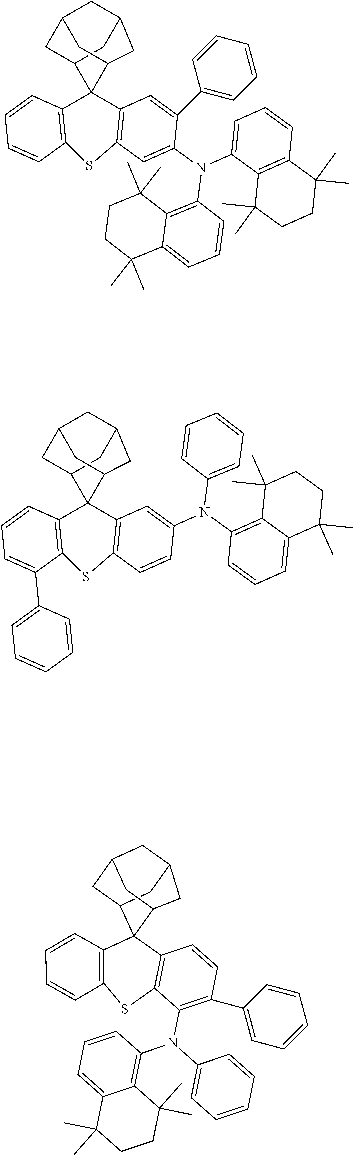 [SMILES: CC1(C)CCC(C)(C)c2c(N(c3cc4c(cc3-c3ccccc3)C3(c5ccccc5S4)C4CC5CC(C4)CC3C5)c3cccc4c3C(C)(C)CCC4(C)C)cccc21 ; CC1(C)CCC(C)(C)c2c(N(c3ccccc3)c3ccc4c(c3)C3(c5cccc(-c6ccccc6)c5S4)C4CC5CC(C4)CC3C5)cccc21 ; CC1(C)CCC(C)(C)c2c(N(c3ccccc3)c3c(-c4ccccc4)ccc4c3Sc3ccccc3C43C4CC5CC(C4)CC3C5)cccc21]

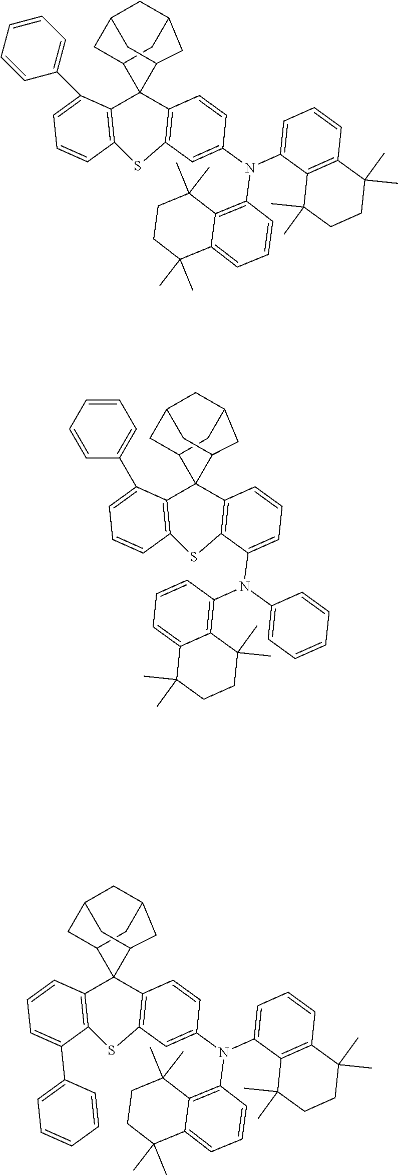 [SMILES: CC1(C)CCC(C)(C)c2c(N(c3ccc4c(c3)Sc3cccc(-c5ccccc5)c3C43C4CC5CC(C4)CC3C5)c3cccc4c3C(C)(C)CCC4(C)C)cccc21 ; CC1(C)CCC(C)(C)c2c(N(c3ccccc3)c3cccc4c3Sc3cccc(-c5ccccc5)c3C43C4CC5CC(C4)CC3C5)cccc21 ; CC1(C)CCC(C)(C)c2c(N(c3ccc4c(c3)Sc3c(-c5ccccc5)cccc3C43C4CC5CC(C4)CC3C5)c3cccc4c3C(C)(C)CCC4(C)C)cccc21]

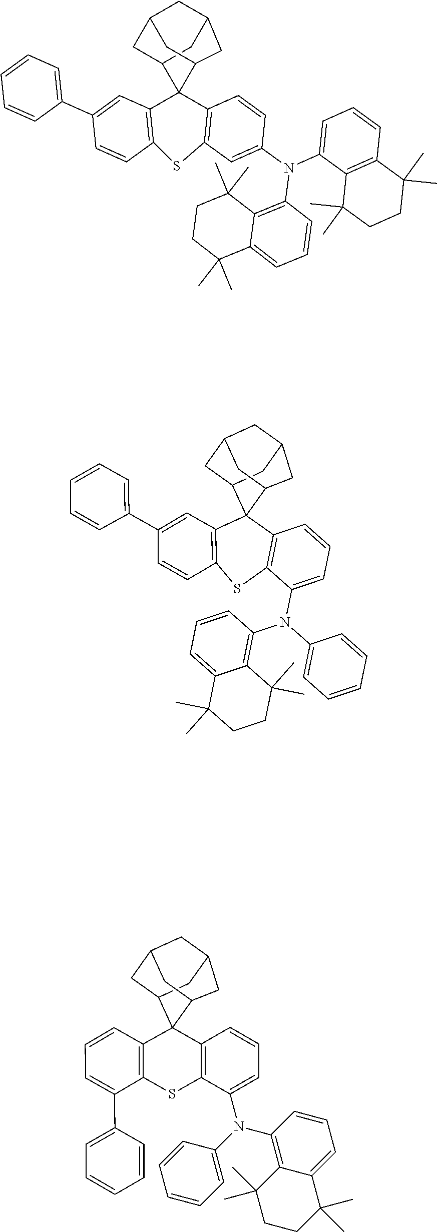 [SMILES: CC1(C)CCC(C)(C)c2c(N(c3ccc4c(c3)Sc3ccc(-c5ccccc5)cc3C43C4CC5CC(C4)CC3C5)c3cccc4c3C(C)(C)CCC4(C)C)cccc21 ; CC1(C)CCC(C)(C)c2c(N(c3ccccc3)c3cccc4c3Sc3ccc(-c5ccccc5)cc3C43C4CC5CC(C4)CC3C5)cccc21 ; CC1(C)CCC(C)(C)c2c(N(c3ccccc3)c3cccc4c3Sc3c(-c5ccccc5)cccc3C43C4CC5CC(C4)CC3C5)cccc21]

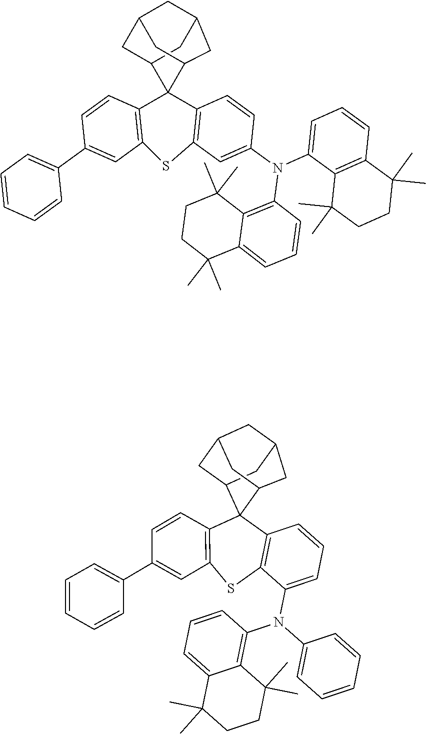 [SMILES: CC1(C)CCC(C)(C)c2c(N(c3ccc4c(c3)Sc3cc(-c5ccccc5)ccc3C43C4CC5CC(C4)CC3C5)c3cccc4c3C(C)(C)CCC4(C)C)cccc21 ; CC1(C)CCC(C)(C)c2c(N(c3ccccc3)c3cccc4c3Sc3cc(-c5ccccc5)ccc3C43C4CC5CC(C4)CC3C5)cccc21]

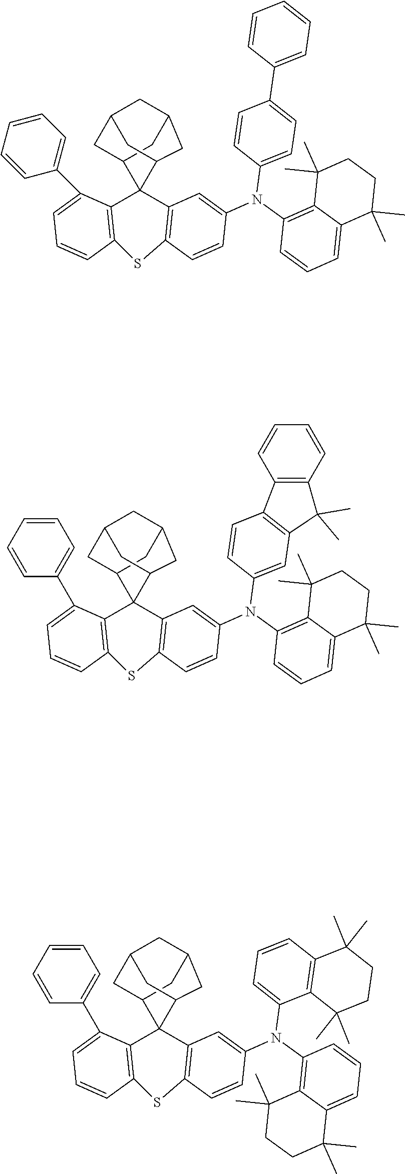 [SMILES: CC1(C)CCC(C)(C)c2c(N(c3ccc(-c4ccccc4)cc3)c3ccc4c(c3)C3(c5c(cccc5-c5ccccc5)S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5c(cccc5-c5ccccc5)S4)C4CC5CC(C4)CC3C5)cccc21 ; CC1(C)CCC(C)(C)c2c(N(c3ccc4c(c3)C3(c5c(cccc5-c5ccccc5)S4)C4CC5CC(C4)CC3C5)c3cccc4c3C(C)(C)CCC4(C)C)cccc21]

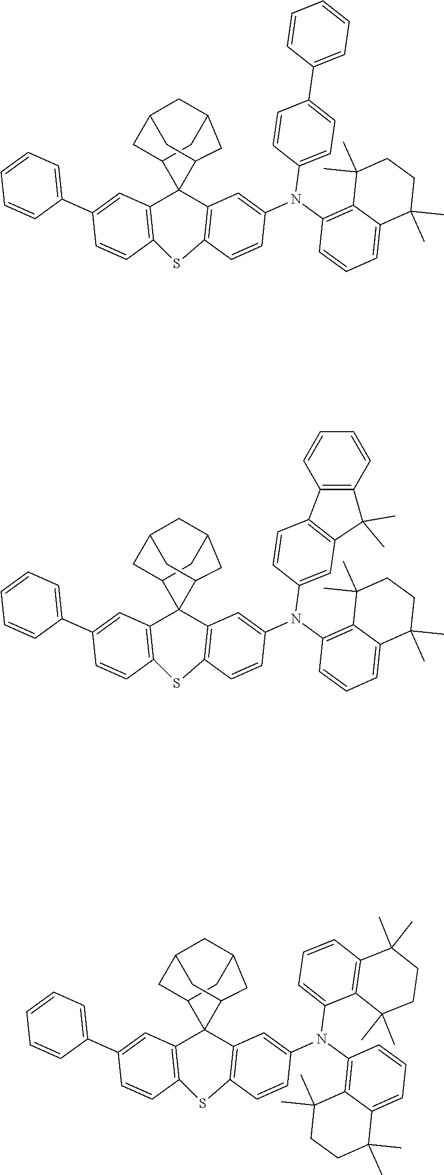 [SMILES: CC1(C)CCC(C)(C)c2c(N(c3ccc(-c4ccccc4)cc3)c3ccc4c(c3)C3(c5cc(-c6ccccc6)ccc5S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cc(-c6ccccc6)ccc5S4)C4CC5CC(C4)CC3C5)cccc21 ; CC1(C)CCC(C)(C)c2c(N(c3ccc4c(c3)C3(c5cc(-c6ccccc6)ccc5S4)C4CC5CC(C4)CC3C5)c3cccc4c3C(C)(C)CCC4(C)C)cccc21]

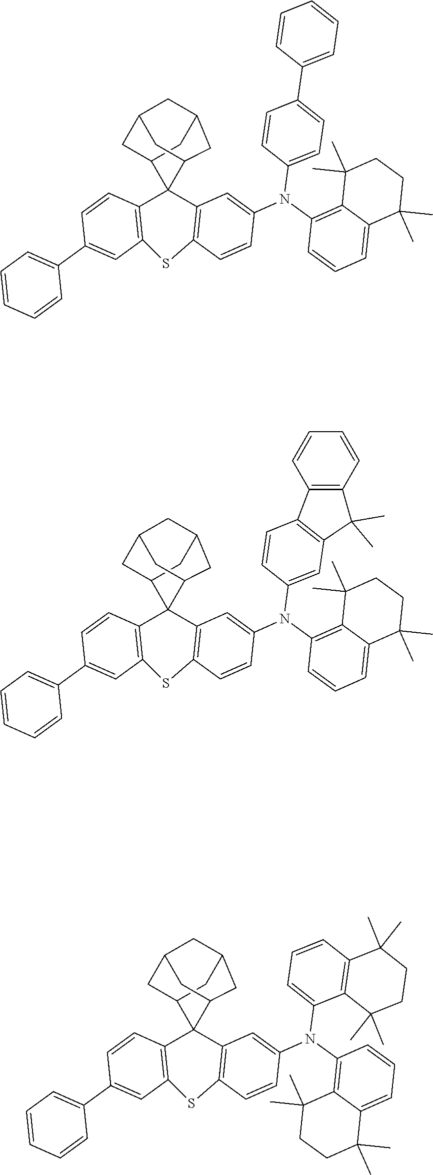 [SMILES: CC1(C)CCC(C)(C)c2c(N(c3ccc(-c4ccccc4)cc3)c3ccc4c(c3)C3(c5ccc(-c6ccccc6)cc5S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5ccc(-c6ccccc6)cc5S4)C4CC5CC(C4)CC3C5)cccc21 ; CC1(C)CCC(C)(C)c2c(N(c3ccc4c(c3)C3(c5ccc(-c6ccccc6)cc5S4)C4CC5CC(C4)CC3C5)c3cccc4c3C(C)(C)CCC4(C)C)cccc21]

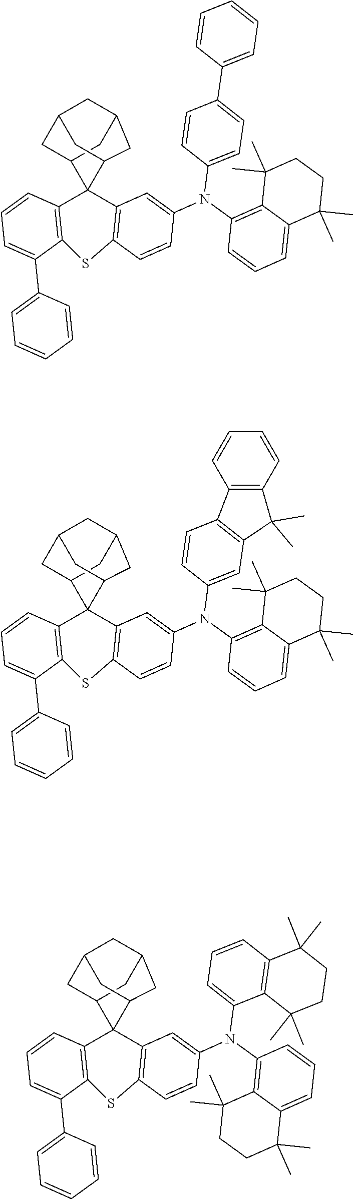 [SMILES: CC1(C)CCC(C)(C)c2c(N(c3ccc(-c4ccccc4)cc3)c3ccc4c(c3)C3(c5cccc(-c6ccccc6)c5S4)C4CC5CC(C4)CC3C5)cccc21 ; CC1(C)CCC(C)(C)c2c(N(c3ccc4c(c3)C(C)(C)c3ccccc3-4)c3ccc4c(c3)C3(c5cccc(-c6ccccc6)c5S4)C4CC5CC(C4)CC3C5)cccc21 ; CC1(C)CCC(C)(C)c2c(N(c3ccc4c(c3)C3(c5cccc(-c6ccccc6)c5S4)C4CC5CC(C4)CC3C5)c3cccc4c3C(C)(C)CCC4(C)C)cccc21]

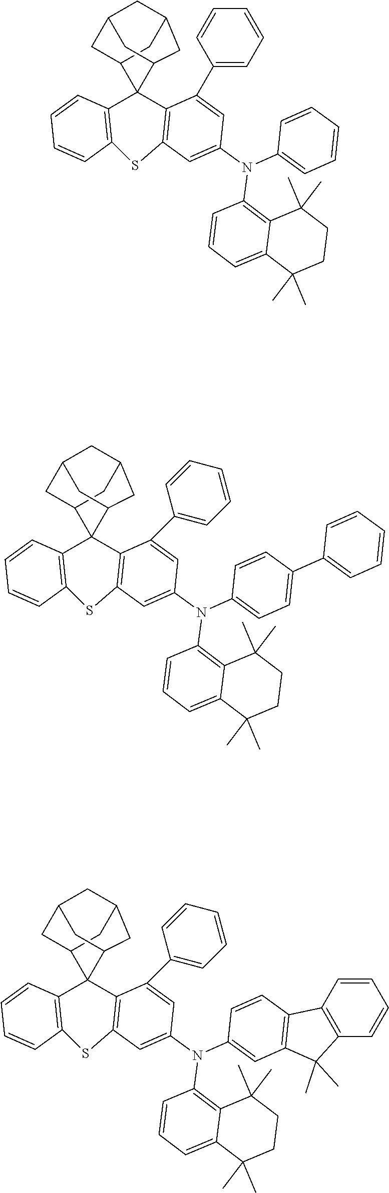 [SMILES: CC1(C)CCC(C)(C)c2c(N(c3ccccc3)c3cc4c(c(-c5ccccc5)c3)C3(c5ccccc5S4)C4CC5CC(C4)CC3C5)cccc21 ; CC1(C)CCC(C)(C)c2c(N(c3ccc(-c4ccccc4)cc3)c3cc4c(c(-c5ccccc5)c3)C3(c5ccccc5S4)C4CC5CC(C4)CC3C5)cccc21 ; CC1(C)CCC(C)(C)c2c(N(c3cc4c(c(-c5ccccc5)c3)C3(c5ccccc5S4)C4CC5CC(C4)CC3C5)c3ccc4c(c3)C(C)(C)c3ccccc3-4)cccc21]

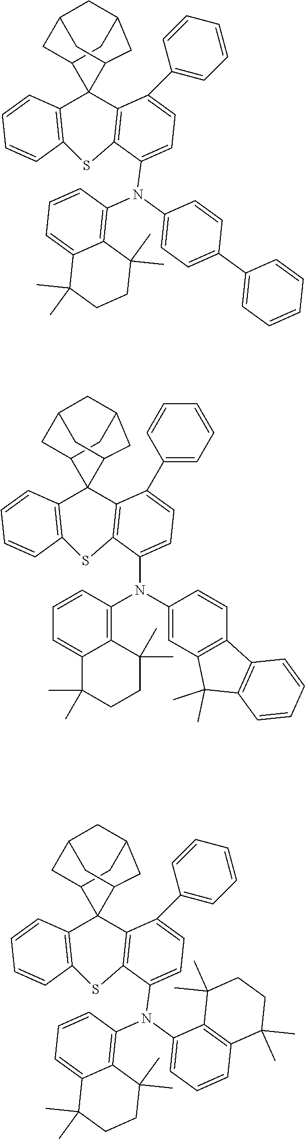 [SMILES: CC1(C)CCC(C)(C)c2c(N(c3ccc(-c4ccccc4)cc3)c3ccc(-c4ccccc4)c4c3Sc3ccccc3C43C4CC5CC(C4)CC3C5)cccc21 ; CC1(C)CCC(C)(C)c2c(N(c3ccc4c(c3)C(C)(C)c3ccccc3-4)c3ccc(-c4ccccc4)c4c3Sc3ccccc3C43C4CC5CC(C4)CC3C5)cccc21 ; CC1(C)CCC(C)(C)c2c(N(c3ccc(-c4ccccc4)c4c3Sc3ccccc3C43C4CC5CC(C4)CC3C5)c3cccc4c3C(C)(C)CCC4(C)C)cccc21]

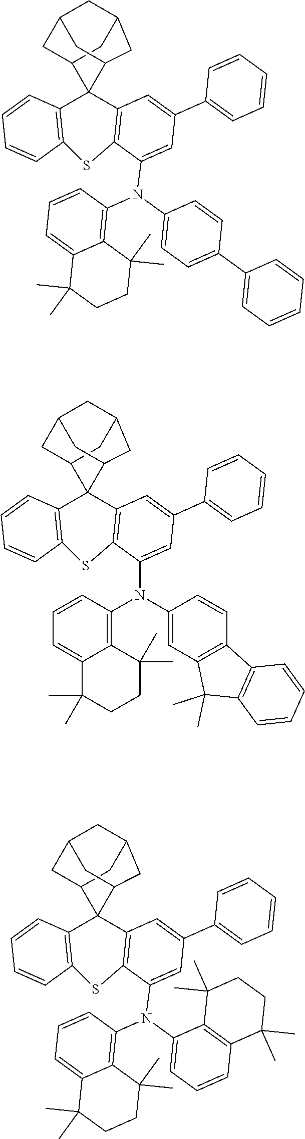 [SMILES: CC1(C)CCC(C)(C)c2c(N(c3ccc(-c4ccccc4)cc3)c3cc(-c4ccccc4)cc4c3Sc3ccccc3C43C4CC5CC(C4)CC3C5)cccc21 ; CC1(C)CCC(C)(C)c2c(N(c3ccc4c(c3)C(C)(C)c3ccccc3-4)c3cc(-c4ccccc4)cc4c3Sc3ccccc3C43C4CC5CC(C4)CC3C5)cccc21 ; CC1(C)CCC(C)(C)c2c(N(c3cc(-c4ccccc4)cc4c3Sc3ccccc3C43C4CC5CC(C4)CC3C5)c3cccc4c3C(C)(C)CCC4(C)C)cccc21]

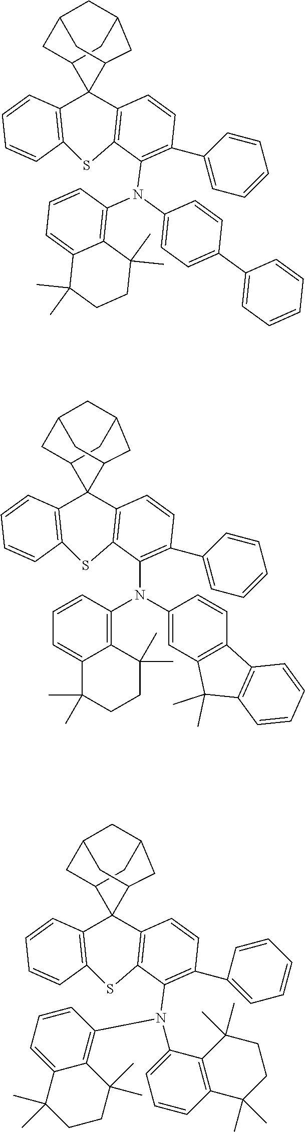 [SMILES: CC1(C)CCC(C)(C)c2c(N(c3ccc(-c4ccccc4)cc3)c3c(-c4ccccc4)ccc4c3Sc3ccccc3C43C4CC5CC(C4)CC3C5)cccc21 ; CC1(C)CCC(C)(C)c2c(N(c3ccc4c(c3)C(C)(C)c3ccccc3-4)c3c(-c4ccccc4)ccc4c3Sc3ccccc3C43C4CC5CC(C4)CC3C5)cccc21 ; CC1(C)CCC(C)(C)c2c(N(c3cccc4c3C(C)(C)CCC4(C)C)c3c(-c4ccccc4)ccc4c3Sc3ccccc3C43C4CC5CC(C4)CC3C5)cccc21]

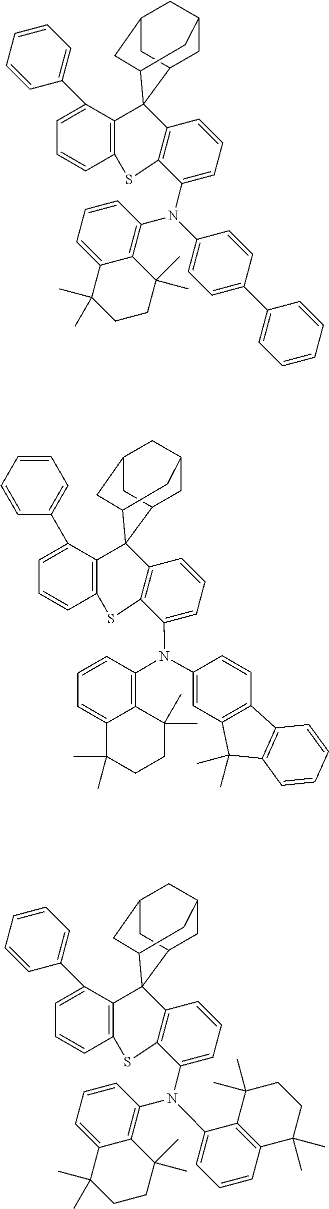 [SMILES: CC1(C)CCC(C)(C)c2c(N(c3ccc(-c4ccccc4)cc3)c3cccc4c3Sc3cccc(-c5ccccc5)c3C43C4CC5CC(C4)CC3C5)cccc21 ; CC1(C)CCC(C)(C)c2c(N(c3ccc4c(c3)C(C)(C)c3ccccc3-4)c3cccc4c3Sc3cccc(-c5ccccc5)c3C43C4CC5CC(C4)CC3C5)cccc21 ; CC1(C)CCC(C)(C)c2c(N(c3cccc4c3Sc3cccc(-c5ccccc5)c3C43C4CC5CC(C4)CC3C5)c3cccc4c3C(C)(C)CCC4(C)C)cccc21]

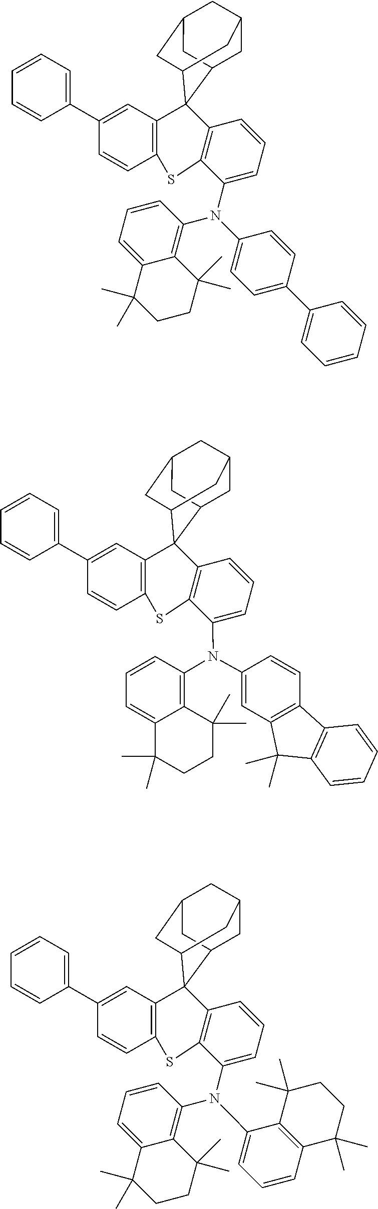 [SMILES: CC1(C)CCC(C)(C)c2c(N(c3ccc(-c4ccccc4)cc3)c3cccc4c3Sc3ccc(-c5ccccc5)cc3C43C4CC5CC(C4)CC3C5)cccc21 ; CC1(C)CCC(C)(C)c2c(N(c3ccc4c(c3)C(C)(C)c3ccccc3-4)c3cccc4c3Sc3ccc(-c5ccccc5)cc3C43C4CC5CC(C4)CC3C5)cccc21 ; CC1(C)CCC(C)(C)c2c(N(c3cccc4c3Sc3ccc(-c5ccccc5)cc3C43C4CC5CC(C4)CC3C5)c3cccc4c3C(C)(C)CCC4(C)C)cccc21]

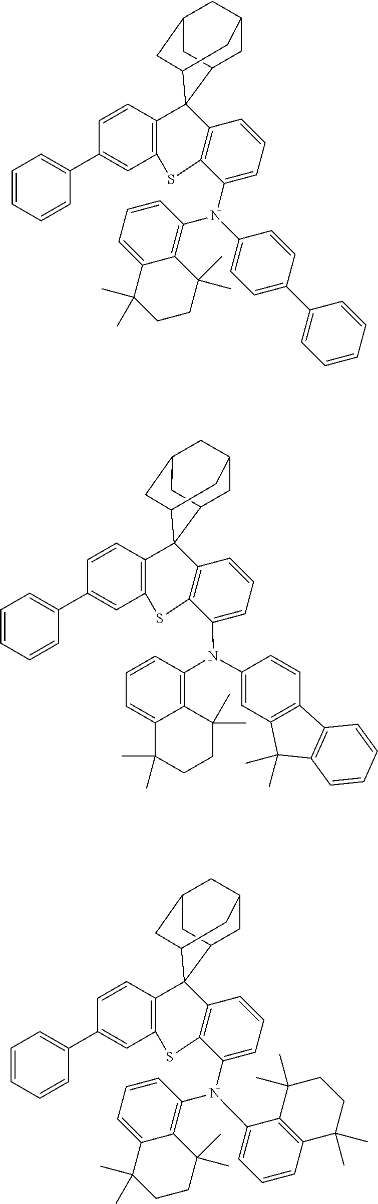 [SMILES: CC1(C)CCC(C)(C)c2c(N(c3ccc(-c4ccccc4)cc3)c3cccc4c3Sc3cc(-c5ccccc5)ccc3C43C4CC5CC(C4)CC3C5)cccc21 ; CC1(C)CCC(C)(C)c2c(N(c3ccc4c(c3)C(C)(C)c3ccccc3-4)c3cccc4c3Sc3cc(-c5ccccc5)ccc3C43C4CC5CC(C4)CC3C5)cccc21 ; CC1(C)CCC(C)(C)c2c(N(c3cccc4c3Sc3cc(-c5ccccc5)ccc3C43C4CC5CC(C4)CC3C5)c3cccc4c3C(C)(C)CCC4(C)C)cccc21]

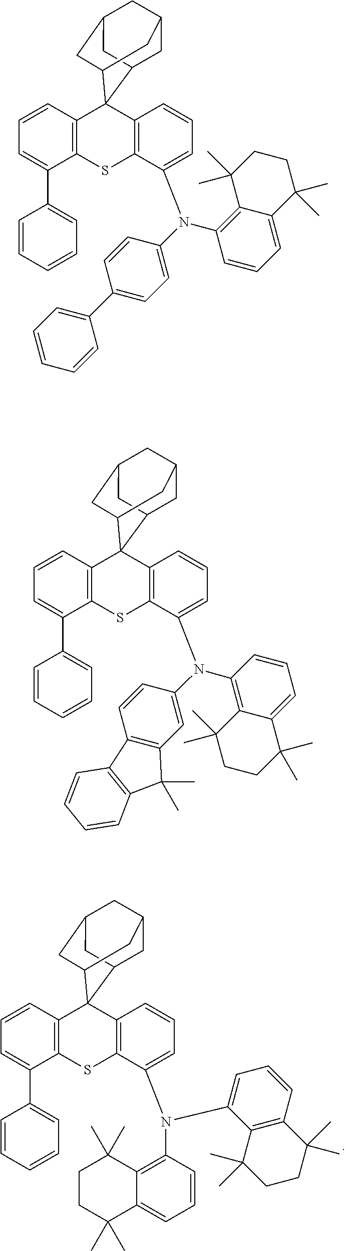 [SMILES: CC1(C)CCC(C)(C)c2c(N(c3ccc(-c4ccccc4)cc3)c3cccc4c3Sc3c(-c5ccccc5)cccc3C43C4CC5CC(C4)CC3C5)cccc21 ; CC1(C)CCC(C)(C)c2c(N(c3ccc4c(c3)C(C)(C)c3ccccc3-4)c3cccc4c3Sc3c(-c5ccccc5)cccc3C43C4CC5CC(C4)CC3C5)cccc21 ; CC1(C)CCC(C)(C)c2c(N(c3cccc4c3Sc3c(-c5ccccc5)cccc3C43C4CC5CC(C4)CC3C5)c3cccc4c3C(C)(C)CCC4(C)C)cccc21]

8. An organic light emitting device comprising a first electrode; a second electrode that is provided opposite to the first electrode; and one or more organic material layers that are provided between the first electrode and the second electrode, wherein the one or more organic material layers comprise the compound as set forth in claim 1.

9. The organic light emitting device of claim 8, wherein
the organic material layer is an electron blocking layer or a hole transport layer.
